(19) United States
(12) Patent Application Publication
(10) Pub. No.: US 2014/0315886 A1
(43) Pub. Date: Oct. 23, 2014

(54) QUINAZOLINES AS THERAPEUTIC COMPOUNDS AND RELATED METHODS OF USE

(71) Applicants: Masaki Suzuki, Osaka-shi (JP); Kazumi Kondo, Osaka-shi (JP); Muneaki Kurimura, Osaka-shi (JP); Krishna Reddy Valluru, Hyderabad (IN); Akira Takahahi, Osaka-shi (JP); Takeshi Kuroda, Osaka-shi (JP); Haruka Takahashi, Osaka-shi (JP); Tae Fukushima, Osaka-shi (JP); Shin Miyamura, Osaka-shi (JP); Indranath Ghosh, Belmont, MA (US); Abhishek Dogra, Somerville, MA (US); Geraldine Harriman, Charlestown, RI (US); Amy Elder, Arlington, MA (US); Satoshi Shimiza, Osaka-shi (JP); Kevin J. Hodgetts, Waltham, MA (US); Jason S. Newcom, Norfolk, MA (US)

(72) Inventors: Masaki Suzuki, Osaka-shi (JP); Kazumi Kondo, Osaka-shi (JP); Muneaki Kurimura, Osaka-shi (JP); Krishna Reddy Valluru, Hyderabad (IN); Akira Takahahi, Osaka-shi (JP); Takeshi Kuroda, Osaka-shi (JP); Haruka Takahashi, Osaka-shi (JP); Tae Fukushima, Osaka-shi (JP); Shin Miyamura, Osaka-shi (JP); Indranath Ghosh, Belmont, MA (US); Abhishek Dogra, Somerville, MA (US); Geraldine Harriman, Charlestown, RI (US); Amy Elder, Arlington, MA (US); Satoshi Shimiza, Osaka-shi (JP); Kevin J. Hodgetts, Waltham, MA (US); Jason S. Newcom, Norfolk, MA (US)

(73) Assignee: OTSUKA PHARMACEUTICAL CO., LTD., Tokyo (JP)

(21) Appl. No.: 14/129,805

(22) PCT Filed: Jun. 28, 2012
(86) PCT No.: PCT/US12/44636

§ 371 Date: Jun. 9, 2014

Related U.S. Application Data
(60) Provisional application No. 61/502,592, filed on Jun. 29, 2011.

(51) Int. Cl. C07D 405/14; C07D 401/04; C07D 403/04; C07D 413/14; C07D 417/14; C07D 401/14; A61K 31/517; A61K 45/06

(52) U.S. Cl. CPC C07D 405/14; A61K 31/517; C07D 401/04; A61K 45/06; C07D 413/14; C07D 417/14; C07D 401/14; C07D 403/04
USPC 514/210.21; 514/266.2; 514/266.21; 544/284

(57) ABSTRACT

Methods of treating disorders using compounds (I) that modulate stri-atal-enriched tyrosine phosphatase (STEP) are described herein. Exemplary disorders include schizophrenia and cognitive deficit. Formula (I).

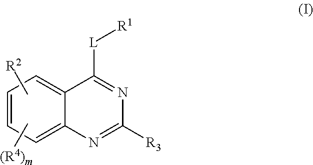

### BACKGROUND OF INVENTION

Tyrosine phosphorylation of synaptic receptors and signaling molecules regulates synaptic activity. A number of protein tyrosine phosphatases specifically expressed within the brain have been identified, including STEP (for STriatal-Enriched tyrosine Phosphatase, also known as PTPN5). Recent evidence suggests that STEP plays an important role in synaptic plasticity, for review see (Braithwaite S P, et al., (2006), Trends Neurosci, 29 (8): 452; Baum M L, et al., (2010), Commun Integr Biol, 3 (5): 419). STEP is specifically expressed within neurons of the central nervous system. As its name indicates, the highest expression level is within the striatum. However, more recent work has found that it is expressed at lower levels in multiple brain regions including the neocortex, amygdala, hippocampus, and embryonic spinal cord.

Four groups of proteins that STEP regulates have been identified: the mitogen-activated protein kinases (MAPKs), the tyrosine kinase Fyn, the N-methyl-D-aspartate (NMDA) receptor complex (specifically the NR2B subunit) and AMPA receptors (specifically, GluR2, (Zhang Y, et al., (2008), J Neurosci, 28 (42): 10561)). Three additional new substrates for STEP have also been recently discovered; proline-rich tyrosine kinase 2 (Pyk2; Xu J, et al., (2010), Abstracts of the Society for Neuroscience Meetings), the fragile X mental retardation protein (FMRP) (Goebel-Goody S M, et al., (2010), Abstracts of the Society for Neuroscience Meetings) and the cell-death mediator Bak (Fox J L, et al., (2010), EMBO J, 29 (22): 3853). Tyrosine phosphorylation of one member of the MAPK family, the extracellular signal regulated kinase (ERK), is necessary for the expression and maintenance of synaptic plasticity in many brain regions, and disruption of the ERK pathway leads to a disruption of learning and memory. One of the functions of these src and Pyk2 kinases is to phosphorylate NMDA receptors, thereby modulating their channel conductance properties and facilitating their movement toward the surface of neuronal plasma membranes. Pyk2 and Fyn tyrosine kinases are activated by phosphorylation on tyrosine residues. NR2B phosphorylation on Tyrosine 1452 inhibits the receptor endocytosis. STEP acts as direct or indirect brake of NMDAR mediated signaling by either respectively dephosphorylating NR2B or its associated kinases, Pyk2 and Fyn. Activation of AMPA, NMDA receptors and MAPKs are required for the induction of several forms of long-term potentiation (LTP) and long-term depression (LTD). Hippocampal LTP is increased in transgenic mice model of Alzheimer lacking STEP (Zhang Y, et al., (2010), Proc Natl Acad Sci USA, 107 (44): 19014). NR2B and AMPA receptor surface expression is increased in STEP KO mice. AMPA receptor endocytosis in group I metabotropic glutamate receptor I (mGluR) mediated LTD is mediated by a tyrosine phosphatase. AMPA receptor endocytosis induced by activation of group I mGLuR is blocked in STEP KO mice suggesting that STEP might also control mGluR mediated LTD.

Compounds that inhibit STEP activity should mimic the effects observed with the STEP KO and may be useful for treating conditions mediated by abnormal NMDA-receptor (NMDA-Rs) and/or MAP kinase pathway signaling. Both may mediate cognition, learning and memory, neurogenesis, and may also affect neuronal plasticity, pain perception, mood and anxiety, and neuroendocrine regulation.

Modulation of NMDA-Rs:

STEP decreases the tyrosine phosphorylation level of NMDA-Rs. Less phosphorylated NMDA-Rs have lower conductance states and thus will allow less current and fewer ions to pass. The NMDA-Rs will therefore be functionally less active (Alvestad R M, et al., (2003), J Biol Chem, 278 (13): 11020), which can lead to schizophrenic symptoms. Hypofunction of NMDA-Rs has been liked to schizophrenia. For example, phencyclidine, ketamine, and other noncompetitive antagonists at NMDA-type glutamate receptors can exacerbate symptoms in patients (Lahti A C, et al., (1995), Neuropsychopharmacology, 13 (1): 9) and may produce a range of psychotic symptoms in volunteers that are similar to those of schizophrenic patients. NMDA-R hypofunction is also linked to psychosis and drug addiction (Javitt D C and Zukin S R, (1991), Am J Psychiatry, 148 (10): 1301). Chronic treatment of atypical antipsychotic clozapine and risperidone in mice result in significant increase of phosphorylation of ERK, NR2B and Pyk2 on tyrosine residues recognized by STEP (Carty N C, et al., (2010), Abstracts of the Society for Neuroscience Meetings). Treatment of these anti-psychotics also enhances cAMP and STEP phosphorylation. Since PKA mediated phosphorylation of STEP is know to inactivate STEP, these results suggest that STEP inhibition mediates the beneficial effect of antipsychotic drugs. Recent studies have linked abnormal NMDA-R activity and expression of STEP to the cognitive decline observed in Alzheimer's disease or transgenic mice expressing mutant APP (Tg2576 mice) (Snyder E M, et al., (2005), Nat Neurosci, 8 (8): 1051; Hynd M R, et al., (2004), J Neurochem, 90 (4): 913; Kurup P, et al., (2010), Channels (Austin), 4 (5)). More specifically, STEP KO mice are less susceptible to PCP-induced hyperlocomotion and PCP-induced cognitive deficits in the object recognition tasks (Carty N C, et al., (2010), Abstracts of the Society for Neuroscience Meetings). Compared to the Tg2576 mice expressing STEP, Tg2576 lacking STEP gene showed rescue in their deficits in hyppocampal LTP and in different behavioral cognitive tasks. Altogether, these results suggest that STEP inhibitors might represent a novel class of drugs that can treat both positive symptoms and cognitive deficit associated with schizophrenia.

Medications that modulate glutamatergic neurotransmission via NMDA-Rs may be also effective in treatment for mood and anxiety disorders. Administration of NMDA-R antagonists has anxiolytic effects in rodent models of anxiety (Falls W A, et al., (1992), J Neurosci, 12 (3): 854; Miserendino M J, et al., (1990), Nature, 345 (6277): 716). NMDA-Rs antagonist like ketamine has been shown to be effective in drug-resistant unipolar depression (Machado-Vieira R, et al., (2009), Pharmacol Ther, 123 (2): 143).

Abnormal balance between the activity of NMDA receptors at synaptic (prosurvival linked to ERK activation) and extrasynaptic (proapoptotic linked to p38 activation) sites has been proposed in cellular and mouse model of Huntington Disease (HD) (Milnerwood A J, et al., Neuron, 65 (2): 178). YAC128 mouse model (containing high number of glutamine repeat on huntingtin) of HD showed an increased activity of extrasynaptic NMDA receptors (NR2B subunit) and require p38 and caspase-6 cleavage activation. In YAC128 mice, NR2B synaptic expression is associated with high STEP expression and activity and a reduction in NR2B expression and phosphorylation (Gladding C M, et al., (2010), Abstracts of the Society for Neuroscience Meetings). Extrasynaptic NMDA receptors couple preferentially to excitotoxicity via calpain-mediated cleavage of STEP and activation of p38 (Xu J, et al., (2009), J Neurosci, 29 (29): 9330) Inhibiting STEP activity might therefore shift the balance toward the NMDA receptor/ERK synaptic prosurvival signaling pathway.

Modulation of ERK Pathway:

STEP inhibition may translate into activation of ERK1/2 kinases, for example, in the central nervous system (CNS). Activation of the ERK pathway in the CNS can mediate neurotrophic pathways involved in cellular resilience. ERK signaling directly affects Bak phosphorylation through inhibition of STEP to promote cell survival (Fox J L, et al., (2010), EMBO J, 29 (22): 3853). BDNF and other neurotrophins can block apoptosis and increase cell survival of different type of CNS neurons in vitro and in vivo via stimulation of the ERK pathway. Mood stabilizers effective in bipolar disorder like valproate and lithium may be potent activators of ERK activity. This effect on ERK activation is believed to be responsible for the neurotrophic effects of mood stabilizers observed in vitro or in brains of treated patients with bipolar disorder, for review see (Engel S R, et al., (2009), Mol Psychiatry, 14 (4): 448; Chen G and Manji H K, (2006), Curr Opin Psychiatry, 19 (3): 313; Machado-Vieira R, et al., (2009), Bipolar Disord, 11 Suppl 2 92). In vivo disruption of STEP activity was shown to activate MAPK pathway, leading to significant rescue from neuronal cell death after pilocarpine-induced status epilepticus (Choi Y S, et al., (2007), J Neurosci, 27 (11): 2999). Increasing cellular resilience could therefore limit or reduce neuronal loss in several neurologic disorders. Recent work has suggested a positive role for STEP inhibition in fragile X syndrome (FXS). This disorder results from the mutation of fmr1 gene coding for the fragile X mental retardation protein (FMRP). STEP binds to FMRP and its expression is dysregulated in FXS. FMR KO mice model displayed audiogenic seizures. FMR KO mice lacking STEP gene show a significant reduction of these seizures (Goebel-Goody S M, et al., (2010), Abstracts of the Society for Neuroscience Meetings), suggesting that STEP modulators might be therapeutic approach for FXS.

Various substituted heterocyclic compounds are disclosed in the art. For example, WO 02/062767 discloses quinazoline derivatives; WO 03/000188 discloses quinazolines and uses thereof; WO 2005/042501 discloses norepinephrine reuptake inhibitors for the treatment of central nervous system disorders; WO2006/058201 discloses heterocyclic and bicyclic compounds, compositions and methods; WO 2007/104560 discloses substituted 4-amino-quinazoline derivatives as regulators of metabotropic glutamate receptors and their use for producing drugs; WO 2007/133773 discloses CDKI pathway inhibitors; WO 2008/009078 discloses 4,6-DL- and 2,4,6-trisubstituted quinazoline derivatives useful for treating viral infections; WO 2009/000085 discloses quinoline and quinazoline derivatives useful as modulators of gated ion channels; US 2009/0143399 discloses protein kinase inhibitors; and Japan Publication Number 2007-084494A discloses substituted bicyclic compounds.

### SUMMARY OF INVENTION

Described herein are compounds, pharmaceutical compositions containing the compounds, and methods of using the compounds to treat a disorder, e.g., schizophrenia or cognitive deficit, in a subject. The compounds disclosed herein include quinoline- and quinazoline-containing compounds that modulate (e.g., inhibit) the activity of STEP.

The present invention provides aspects described in items below.

Item 1. A compound of formula (I):

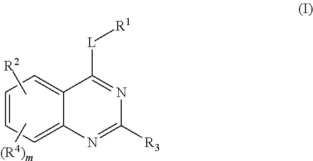

or a salt thereof,

wherein:

m is 0 or 1;

L is a direct bond or NR⁶;

R¹ is hydrogen, C₁-C₈ alkyl, halo C₁-C₈ alkyl, C₁-C₈ alkoxy C₁-C₈ alkyl, hydroxy C₁-C₈ alkyl, amino C₁-C₈ alkyl, oxazolyl, thiazolyl, isoxazolyl, pyridyl, pyrrolopyridyl, oxadiazolyl C₁-C₈ alkyl, pyridyl C₁-C₈ alkyl, oxazolyl C₁-C₈ alkyl, phenyl C₁-C₈ alkyl, —C(O)R^{e}, pyrrolidinyl, azetidinyl, indolinyl, piperidinyl, morpholinyl, piperazinyl, phenyl, C₃-C₈ cycloalkyl, C₃-C₈ cycloalkyl C₁-C₈ alkyl, benzoxazolyl, each of which is optionally substituted with 1-2 R⁷;

R² is C₁-C₈ alkoxy, benzodioxolyl, piperazinyl, halo, phenyl, tetrahydronaphtyl, furyl, oxazolyl, thiazolyl, thiadiazolyl, pyridyl, pyrimidinyl, indolyl, indazolyl, dihydroindazolyl, tetrahydroisoquinolinyl, tetrahydroquinolinyl, dihydrobenzoimidazolyl, dihydrobenzoxazolyl, benzothiazolyl, dihydrobenzothiazolyl, benzothienyl, dihydroisoquinolinyl, isoquinolinyl, benzofuryl, dihydrobenzofuryl, benzodioxolyl, dihydrobenzoxazinyl, dihydrobenzodioxepinyl, tetrahydrobenzoxazepinyl, isoindolinyl, indolinyl, thienyl or dihydrobenzodioxinyl, each of which is optionally substituted with 1-3 R⁹;

R³ is pyridyl, pyrimidinyl, pyrazinyl or pyridazinyl, each of which is optionally substituted with C₁-C₈ alkyl, C₁-C₈ alkoxy, halo, halo C₁-C₈ alkyl, halo C₁-C₈ alkoxy, cyano or —OR^{d};

R⁴ is hydrogen, C₁-C₈ alkyl, C₁-C₈ alkoxy, halo, halo C₁-C₈ alkyl or halo C₁-C₈ alkoxy, each of which is optionally substituted with R¹⁰;

R⁶ is hydrogen or C₁-C₈ alkyl;

R⁷ is C₁-C₈ alkyl, C₂-C₈ alkenyl, C₂-C₈ alkynyl, aryl, heteroaryl, cyclyl, heterocyclyl, arylalkyl, heteroarylalkyl, cyclylalkyl, heterocyclylalkyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with R¹²;

R⁹ is C₁-C₈ alkyl, C₁-C₈ alkoxy, phenyl, pyrazolyl, dihydrobenzoxazolyl, oxazolyl, tetrazolyl, imidazolyl, thiazolyl, C₃-C₈ cycloalkyl, oxetanyl, pyrrolidinyl, morpholinyl, halo, halo C₁-C₈ alkyl, halo C₁-C₈ alkoxy, hydroxy C₁-C₈ alkyl, oxo, cyano, nitro, —C(O)OR^{a}, —C(O)NR^{b}R^{b′}, —NR^{c}C(O)R^{c′}, —NR^{b}R^{b′}, —OR^{d}, —SR^{d′}, —C(O)R^{e} or —S(O)_{q}R^{f}, each of which is optionally substituted with 1-2 R¹²;

R¹⁰ is C₁-C₈ alkoxy, C₂-C₈ alkenyl, C₃-C₈ cycloalkyl, furyl, thienyl, pyrazolyl, morpholinyl, piperazinyl, pyrrolidinyl, piperidinyl, tetrahydropyranyl, cyano, —C(O)NR^{b}R^{b′}, —NR^{c}C(O)R^{c′}, —NR^{b}R^{b′} or —S(O)_{q}R^{f}, each of which is optionally substituted with R¹²;

R¹² is C₁-C₈ alkyl, C₂-C₈ alkenyl, C₂-C₈ alkynyl, aryl, heteroaryl, cyclyl, heterocyclyl, arylalkyl, heteroarylalkyl, cyclylalkyl, heterocyclylalkyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f},each of which is optionally substituted with 1-3 R¹³□

R¹³ is independently C₁-C₈ alkyl, haloalkyl, halo, heterocyclyl, cyclyl, oxo or —C(Y)NR^{b}R^{b′};

each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl; and

q is 1 or 2.

Item 2. The compound according to Item 1 represented by general formula (I) or a salt thereof,

wherein:

if R³ is

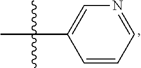 [SMILES: *c1cccnc1]

L is NR⁶, R¹ is benzyl, R⁶ is hydrogen, and R⁴ is hydrogen, then R² is not halo or methoxy;

if R³ is

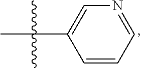 [SMILES: *c1cccnc1]

L is NR⁶, R¹ is phenyl, R⁶ is methyl, and R⁴ is hydrogen, then R² is not halo;

if R³ is

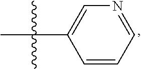 [SMILES: *c1cccnc1]

L is NR⁶, R¹ is para-trifluoromethyl-phenyl, R⁶ is hydrogen, and R⁴ is hydrogen, then R² is not

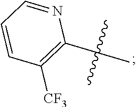 [SMILES: *c1ncccc1C(F)(F)F]

if R³ is

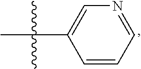 [SMILES: *c1cccnc1]

L is NR⁶, R¹ is indolinyl, R⁶ is hydrogen, and R⁴ is hydrogen, then R² is not chloro; and

if R³ is

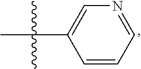 [SMILES: *c1cccnc1]

L is NR⁶, R¹ is dimethylaminomethyl, R⁶ is hydrogen, and R⁴ is methoxy, then R² is not methoxy.
Item 3. The compound according to Item 2 represented by general formula (I) or a salt thereof, provided the compounds in Table X are excluded.
Item 4. The compound according to any one of Items 1 to 3, represented by general formula (I) or a salt thereof,

wherein:

R¹ is C₃-C₈ cycloalkyl, oxazolyl, thiazolyl, isoxazolyl, pyridyl, pyrrolopyridyl, indolinyl, phenyl or benzoxazolyl, each of which is optionally substituted with 1-2 R⁷;

R² is C₁-C₈ alkoxy, piperazinyl, halo or pyrimidinyl, each of which is optionally substituted with 1-3 R⁹;

R³ is pyridyl (e.g, 3-pyridyl);

R⁴ is hydrogen;

R⁶ is hydrogen;

R⁷ is C₁-C₈ alkyl, C₁-C₈ alkoxy, halo, halo C₁-C alkyl, cyano, nitro or —C(O)NR^{b}R^{b′} or —NR^{c}C(O)R^{c′};

R⁹ is C₁-C₈ alkyl, C₁-C₈ alkoxy, halo, cyano, nitro, —C(O)NR^{b}R^{b′} or —NR^{c}C(O)R^{c′}, —NR^{b}R^{b′};

each R^{a}, R^{b}, R^{b′}, R^{c}, and R^{c′} is independently hydrogen, C₁-C₈ alkyl or C₁-C₈ alkoxy; and

q is 1 or 2.

Item 5. The compound according to any one of Items 1 to 3, represented by general formula (I) or a salt thereof,

wherein:

R¹ is C₁-C₈ alkyl, phenyl or pyridyl C₁-C₈ alkyl, each of which is optionally substituted with 1-2 R⁷;

R² is C₁-C₈ alkoxy or phenyl, each of which is optionally substituted with 1-3 R⁹;

R³ is pyrimidinyl, pyrazinyl or pyridazinyl;

R⁴ is hydrogen or C₁-C₈ alkoxy;

R⁶ is hydrogen;

R⁷ is C₁-C₈ alkyl or —C(O)NH₂;

R⁹ is halo; and q is 1 or 2.

Item 6. The compound according to any one of Items 1 to 3, represented by general formula (I) or a salt thereof,

wherein:

m is 0 or 1;

R¹ is hydrogen, C₁-C₈ alkyl, halo C₁-C₈ alkyl, C₁-C₈ alkoxy C₁-C₈ alkyl, hydroxyl C₁-C₈ alkyl, amino C₁-C₈ alkyl, oxazolyl, thiazolyl, isoxazolyl, pyridyl, pyrrolopyridyl, oxadiazolyl C₁-C₈ alkyl, pyridyl C₁-C₈ alkyl, oxazolyl C₁-C₈ alkyl, phenyl C₁-C₈ alkyl, —C(O)R^{e}, C₃-C₈ cycloalkyl, C₃-C₈ cycloalkyl C₁-C₈ alkyl, pyrrolidinyl, azetidinyl, indolinyl, piperidinyl, morpholinyl or piperazinyl, each of which is optionally substituted with 1-2 R⁷;

R² is phenyl, tetrahydronaphthyl, furyl, oxazolyl, thiazolyl, thiadiazolyl, pyridyl, pyrimidinyl, indolyl, indazolyl, dihydroindazolyl, tetrahydroisoquinolinyl, tetrahydroquinolinyl, dihydrobenzoimidazolyl, dihydrobenzoxazolyl, benzothiazolyl, dihydrobenzothiazolyl, benzothienyl, dihydroisoquinolinyl, isoquinolinyl, benzofuryl, dihydrobenzofuryl, benzodioxolyl, dihydrobenzoxazinyl, dihydrobenzodioxepinyl, tetrahydrobenzoxazepinyl, isoindolinyl, indolinyl, thienyl or dihydrobenzodioxinyl, each of which is optionally substituted with 1-3 R⁹;

R³ is pyridyl (e.g, 3-pyridyl), each of which is optionally substituted with C₁-C₈ alkyl, C₁-C₈ alkoxy, halo, halo C₁-C₈ alkyl, halo C₁-C₈ alkoxy, cyano or —OR^{d};

R⁴ is hydrogen, C₁-C₈ alkyl, C₁-C₈ alkoxy, halo, halo C₁-C₈ alkyl or halo C₁-C₈ alkoxy, each of which is optionally substituted with R¹⁰;

R⁶ is hydrogen or C₁-C₈ alkyl;

R⁷ is C₁-C₈ alkyl, C₁-C₈ alkoxy, pyrazolyl, pyridyl, C₃-C₈ cycloalkyl, halo, halo C₁-C₈ alkyl, halo C₁-C₈ alkoxy, C₁-C₈ alkylamino, di C₁-C₈ alkylamino, di C₁-C₈ alkyl amino C₁-C₈ alkyl, oxo, nitro, —C(O)NR^{b}R^{b′}, —NR^{c}C(O)R^{c′} or —C(O)R^{e}, each of which is optionally substituted with R¹²;

R⁹ is C₁-C₈ alkyl, C₁-C₈ alkoxy, phenyl, pyrazolyl, dihydrobenzoxazolyl, oxazolyl, tetrazolyl, imidazolyl, thiazolyl C₃-C₈ cycloalkyl, oxetanyl, pyrrolidinyl, morpholinyl, halo, halo C₁-C₈ alkyl, halo C₁-C₈ alkoxy, hydroxyl C₁-C₈ alkyl, oxo, cyano, nitro, —C(O)OR^{a}, —C(O)NR^{b}R^{b′}, —NR^{c}C(O)R^{c′}, —NR^{b}R^{b′}, —OR^{d}, —SR^{d′}, —C(O)R^{e} or —S(O)_{q}R^{f}, each of which is optionally substituted with 1-2 R¹²;

R¹⁰ is C₁-C₈ alkoxy, C₂-C₈ alkenyl, C₃-C₈ cycloalkyl, furyl, thienyl, pyrazolyl, morpholinyl, piperazinyl, pyrrolidinyl, piperidinyl, tetrahydropyranyl, cyano, —C(O)NR^{b}R^{b′}, —NR^{c}C(O)R^{c′}, —NR^{b}R^{b′} or —S(O)_{q}R^{f}, each of which is optionally substituted with R¹²;

R¹² is C₁-C₈ alkyl, C₁-C₈ alkoxy, halo, halo C₁-C₈ alkyl, silyl C₁-C₈ alkoxy, silyl C₁-C₈ alkoxy C₁-C₈ alkyl, oxo, thioxo, cyano, nitro, —C(O)OR^{a}, —C(O)NR^{b}R^{b′}, —NR^{c}C(O)R^{c′}, —NR^{b}R^{b′}, —OR^{d} or —C(O)R^{e}□

each R^{a}, R^{b}, R^{b′}, R^{c}, R^{c′}, R^{d}, R^{d′}, R^{e} and R^{f} is independently hydrogen, amino, C₁-C₈ alkyl, C₁-C₈ alkoxy, C₂-C₈ alkenyl, C₁-C₈ alkoxy C₁-C₈ alkyl, C₃-C₈ cycloalkyl, tetrahydropyranyl, morpholinyl, thiadiazolyl or thiazolyl; and

q is 1 or 2.

Item 7. The compound of Item 6, wherein R² is phenyl.
Item 8. A compound of formula (II):

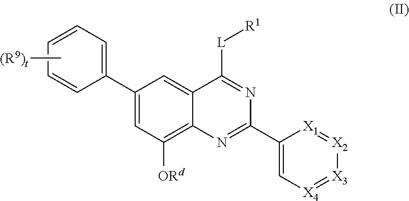

or a salt thereof,

wherein:

L is a direct bond or NR⁶;

one or two of X¹, X², X³, and X⁴ are N and the others are CH,

R¹ is hydrogen, C₁-C₈ alkyl, C₂-C₈ alkenyl, C₂-C₈ alkynyl, alkoxyalkyl, hydroxyalkyl, heteroaryl, heteroarylalkyl, arylalkyl, —C(Y)R^{e}, cyclyl, cyclylalkyl or heterocyclyl, each of which is optionally substituted with 1-3 R⁷;

R⁶ is hydrogen, C₁-C₈ alkyl, C₂-C₈ alkenyl, C₂-C₈ alkynyl, cyclyl or heterocyclyl, each of which is optionally substituted with 1-3 R¹¹;

R⁷ is C₁-C₈ alkyl, C₂-C₈ alkenyl, C₂-C₈ alkynyl, aryl, heteroaryl, cyclyl, heterocyclyl, arylalkyl, heteroarylalkyl, cyclylalkyl, heterocyclylalkyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹²; wherein two R⁷ may be taken together with the atoms to which they are attached to form an optionally substituted cyclyl, heterocyclyl, aryl or heteroaryl ring;

R⁹ is C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹²;

t is 1 to 4, wherein two R⁹ may be taken together with the atoms to which they are attached to form an optionally substituted cyclyl, heterocyclyl, aryl or heteroaryl ring;

each R¹¹ and R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹³;

R¹³ is independently C₁-C₈ alkyl, haloalkyl, halo, heterocyclyl, cyclyl, oxo or —C(Y)NR^{b}R^{b′};

Y is independently O or S;

q is 1 or 2; and

each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.
Item 9. The compound of Item 8, wherein if X₂ is N and X₁, X₃, X₄ are CH, is

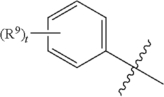

not

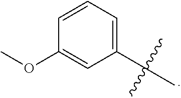 [SMILES: *c1cccc(OC)c1]

Item 10. The compound of Item 8, provided the compounds in Table X are excluded.
Item 11. The compound of any one of Items 8 to 10, wherein X₂ is N, and X₁, X₃, and X₄ are CH.
Item 12. The compound of any one of Items 8 to 10, wherein X₁ and X₃ are N, and X₂ and X₄ are CH.
Item 13. The compound of any one of Items 8 to 12, wherein R^{d} is methyl.
Item 14. The compound of any one of Items 8 to 13, wherein R⁹ is fluoro.
Item 15. A compound of formula (III):

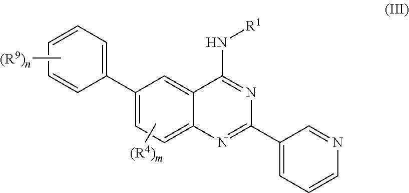

wherein:
R¹ is hydrogen, C₁-C₈ alkyl, halo C₁-C₈ alkyl, C₁-C₈ alkoxy C₁-C₈ alkyl, hydroxy C₁-C₈ alkyl, amino C₁-C₈ alkyl, oxazolyl, thiazolyl, isoxazolyl, pyridyl, pyrrolopyridyl, oxadiazolyl C₁-C₈ alkyl, pyridyl C₁-C₈ alkyl, oxazolyl C₁-C₈ alkyl, phenyl C₁-C₈ alkyl, —C(O)R^{e}, pyrrolidinyl, azetidinyl, indolinyl, piperidinyl, morpholinyl, piperazinyl, phenyl, C₃-C₈ cycloalkyl, C₃-C₈ cycloalkyl C₁-C₈ alkyl, benzoxazolyl, each of which is optionally substituted with 1-2 R⁷;

each R⁴ is independently hydrogen, C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹⁰;

m is 1 or 2;

each R⁷, R⁹, or R¹⁰ is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹² wherein two R⁹ may, together with the ring atoms to which they are attached, form a five or six-membered aryl, heteroaryl, cyclic, or heterocyclic;

n is 1, 2, or 3;

each R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹³;

each R¹³ is independently C₁-C₈ alkyl, haloalkyl, halo, heterocyclyl, cyclyl, oxo or —C(Y)NR^{b}R^{b′};

Y is independently O or S;

q is 1 or 2; and

each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.
Item 16. The compound of Item 15, wherein

if R¹ is methyl or phenyl and R⁴ is methyl, then R⁹ is not fluoro, cyano, or methoxy; if formula (III) is formula (III′):

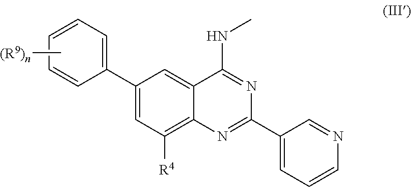

and R⁴ is fluoro or methoxy, then R⁹ is not fluoro or methoxy;
if formula (III) is formula (III″):

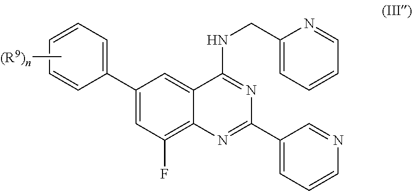

then R⁹ is not fluoro; and
the compound of formula (III) below

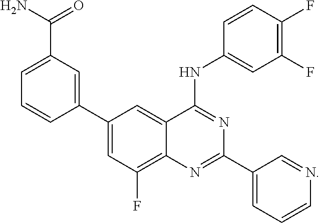 [SMILES: NC(=O)c1cccc(-c2cc(F)c3nc(-c4cccnc4)nc(Nc4ccc(F)c(F)c4)c3c2)c1]

is excluded.
Item 17. The compound of Item 15, provided the compounds in Table X are excluded.
Item 18. The compound of any one of Items 15 to 17, wherein R¹ is C₁-C₈ alkyl.
Item 19. The compound of any one of Items 15 to 18, wherein R⁹ is halo.
Item 20. A compound of formula (IV):

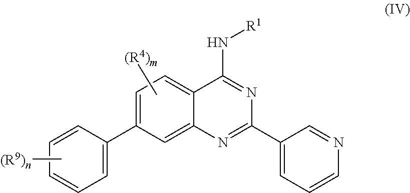

wherein:
R¹ is hydrogen, C₁-C₈ alkyl, halo C₁-C₈ alkyl, C₁-C₈ alkoxy C₁-C₈ alkyl, hydroxy C₁-C₈ alkyl, amino C₁-C₈ alkyl, oxazolyl, thiazolyl, isoxazolyl, pyridyl, pyrrolopyridyl, oxadiazolyl C₁-C₈ alkyl, pyridyl C₁-C₈ alkyl, oxazolyl C₁-C₈ alkyl, phenyl C₁-C₈ alkyl, —C(O)R^{e}, pyrrolidinyl, azetidinyl, indolinyl, piperidinyl, morpholinyl, piperazinyl, phenyl, C₃-C₈ cycloalkyl, C₃-C₈ cycloalkyl C₁-C₈ alkyl, benzoxazolyl, each of which is optionally substituted with 1-2 R⁷;

each R⁴ is independently hydrogen, C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹⁰;

m is 1 or 2;

each R⁷, R⁹, or R¹⁰ is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹², wherein two R⁹ may, together with the ring atoms to which they are attached, form a five or six-membered aryl, heteroaryl, cyclic, or heterocyclic;

n is 1, 2, or 3;

each R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹³;

each R¹³ is independently C₁-C₈ alkyl, haloalkyl, halo, heterocyclyl, cyclyl, oxo or —C(Y)NR^{b}R^{b′};

Y is independently O or S;

q is 1 or 2; and

each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.

Item 21. The compound of Item 20, wherein if R¹ is methyl and R⁴ is methyl, then R⁹ is not fluoro, cyano, or methoxy.
Item 22. The compound of Item 20, provided the compounds in Table X are excluded.
Item 23. The compound any one of Items 20 to 22, wherein R¹ is C₁-C₈ alkyl.
Item 24. The compound any one of Items 20 to 23, wherein R⁴ is fluoro.
Item 25. A compound of formula (V):

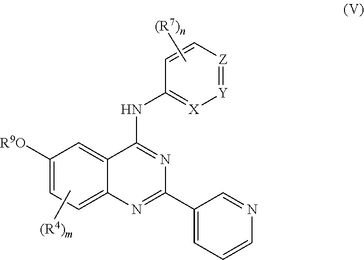

wherein:

one of X, Y, or Z is —N—, the rest being —CH— or —CR⁷—;

each R⁴ is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹⁰;

m is 0, 1, or 2;

each R⁷ or R¹⁰ is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹², wherein two R⁷ may, together with the ring to which they are attached, form a five or six-membered aryl or heteroaryl;

n is 0, 1, 2, or 3;

R⁹ is —CH₃ or —CH₂CH₃;

each R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹³;

each R¹³ is independently C₁-C₈ alkyl, haloalkyl, halo, heterocyclyl, cyclyl, oxo or —C(Y)NR^{b}R^{b′};

Y is independently O or S;

q is 1 or 2; and

each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.

Item 26. The compound of Item 25, wherein the compound is not

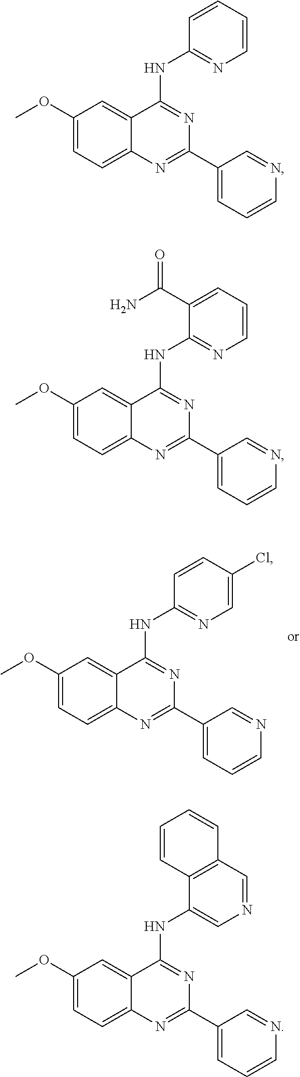 [SMILES: COc1ccc2nc(-c3cccnc3)nc(Nc3ccccn3)c2c1 ; COc1ccc2nc(-c3cccnc3)nc(Nc3ncccc3C(N)=O)c2c1 ; COc1ccc2nc(-c3cccnc3)nc(Nc3ccc(Cl)cn3)c2c1 ; COc1ccc2nc(-c3cccnc3)nc(Nc3cncc4ccccc34)c2c1]

Item 27. The compound of Item 25, provided the compounds in Table X are excluded.
Item 28. The compound of any one of Items 25 to 27, wherein R⁷ is halo.
Item 29. The compound of any one of Items 25 to 28, wherein m is 0.
Item 30. A compound of formula (VI):

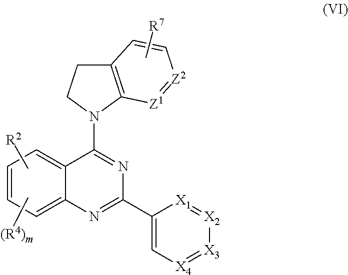

or a salt thereof,
wherein:
one or two of X¹, X², X³, and X⁴ are N and the others are CH;
Z₁ and Z₂ are independently N or CH;
m is 1, 2 or 3;

R² is halo, —OR^{d}, aryl or heteroaryl, wherein the aryl or heteroaryl is optionally substituted with 1-5 R⁹;

each R⁴ is independently hydrogen, C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹⁰;

each R⁷, R⁹, and R¹⁰ is independently C₁-C₈ alkyl, C₂-C₈ alkenyl, C₂-C₈ alkynyl, aryl, heteroaryl, cyclyl, heterocyclyl, arylalkyl, heteroarylalkyl, cyclylalkyl, heterocyclylalkyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹²;

each R¹² is independently C₁-C₈ alkyl, C₂-C₈ alkenyl, C₂-C₈ alkynyl, aryl, heteroaryl, cyclyl, heterocyclyl, arylalkyl, heteroarylalkyl, cyclylalkyl, heterocyclylalkyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f},each of which is optionally substituted with 1-3 R¹³;

R¹³ is independently C₁-C₈ alkyl, haloalkyl, halo, heterocyclyl, cyclyl, oxo or —C(Y)NR^{b}R^{b′};

Y is independently O or S;

q is 1 or 2; and

each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.
Item 31. The compound of Item 30, wherein if Z₁ and Z₂ are both CH, R² is not —Cl or —OR^{d}.
Item 32. The compound of Item 30, provided the compounds in Table X are excluded.
Item 33. The compound of any one of Items 30 to 32, wherein Z₁ is N.
Item 34. The compound of any one of Items 30 to 33, wherein R² is aryl.
Item 35. The compound of any one of Items 30 to 33, wherein R² is —Br or —I.
Item 36. The compound of any one of Items 30 to 35, wherein X₂ is N, and X₁, X₃, and X₄ are CH.
Item 37. A compound of formula (VII):

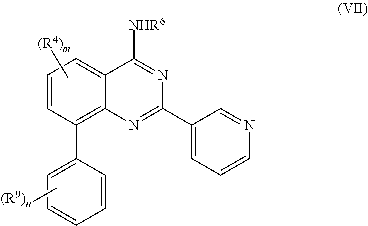

or a salt thereof,
wherein:
m is 1, 2 or 3;
n is 1, 2, 3 or 4;

each R⁴ is independently hydrogen, C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹⁰;

R⁶ is hydrogen, C₁-C₈ alkyl, C₂-C₈ alkenyl, or C₂-C₈ alkynyl, each of which is optionally substituted with 1-3 R¹¹;

each R⁹ and R¹⁰ is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹²;

each R¹¹ and R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹³;

R¹³ is independently C₁-C₈ alkyl, haloalkyl, halo, heterocyclyl, cyclyl, oxo or —C(Y)NR^{b}R^{b′};

Y is independently O or S;

q is 1 or 2; and

each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.

Item 38. The compound of Item 37, wherein if R⁴ is hydrogen,

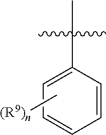

is not

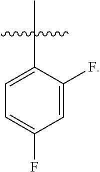 [SMILES: *c1ccc(F)cc1F]

Item 39. The compound of Item 37, provided the compound is not in Table X.
Item 40. The compound of any one of Items 37 to 39, wherein R⁴ is —OCH₃.
Item 41. The compound of any one of Items 37 to 40, wherein R⁹ is —F.
Item 42. A compound of formula (VIII):

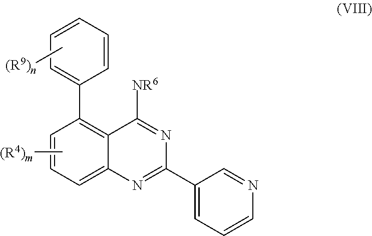

or a salt thereof,
wherein:
m is 1, 2 or 3;
n is 1, 2, 3 or 4;

each R⁴ is independently hydrogen, C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹⁰;

R⁶ is hydrogen, C₁-C₈ alkyl, C₂-C₈ alkenyl, or C₂-C₈ alkynyl, each of which is optionally substituted with 1-3 R¹¹;

each R⁹ and R¹⁰ is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹²;

each R¹¹ and R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹³;

R¹³ is independently C₁-C₈ alkyl, haloalkyl, halo, heterocyclyl, cyclyl, oxo or —C(Y)NR^{b}R^{b′};

Y is independently O or S;

q is 1 or 2; and

each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.

Item 43. The compound of Item 42, provided the compound is not in Table X.
Item 44. The compound of Item 42 or 43, wherein R⁹ is —F.
Item 45. A compound of formula (IX) or (IX′):

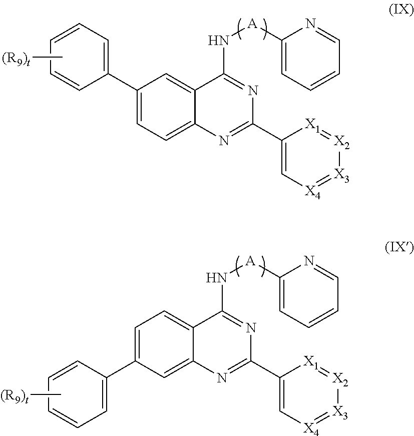

or a salt thereof,
wherein:
A is C₁-C₄ alkylene, optionally substituted with R¹¹;
one or two of X¹, X², X³, and X⁴ are N and the others are CH,

R⁹ is C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹²;

t is 1 to 4, wherein two R⁹ may be taken together with the ring atoms to which they are attached to form an optionally substituted cyclyl, heterocyclyl, aryl or heteroaryl ring;

each R¹¹ and R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹³;

R¹³ is independently C₁-C₈ alkyl, haloalkyl, halo, heterocyclyl, cyclyl, oxo or —C(Y)NR^{b}R^{b′};

alternatively, R¹³ on R¹¹ may connect to the carbon atom of A to which R¹¹ bonds to form a C3-6 cycloalkyl. Y is independently O or S;

q is 1 or 2; and

each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.
Item 46. The compound of Item 45, wherein if X₂ is N and X₁, X₃, X₄ are CH, R⁹ is not —F or —OR^{d}.
Item 47. The compound of Item 45, provided the compound is not in Table X.
Item 48. The compound of any one of Items 45 to 47, wherein A is —CH₂—.
Item 49. The compound of any one of Items 45 to 47, wherein A is —C(CH₃)H—.
Item 50. The compound of any one of Items 45 to 49, wherein R⁹ is —F.
Item 51. A compound disclosed herein.
Item 52. The compound according to Item 8, wherein

R¹ is C₁-C₈ alkyl, halo C₁-C₈ alkyl, C₁-C₈ alkoxy C₁-C₈ alkyl, hydroxyl C₁-C₈ alkyl, amino C₁-C₈ alkyl, oxadiazolyl C₁-C₈ alkyl, oxazolyl C₁-C₈ alkyl, —C(O)R^{e}, C₃-C₈ cycloalkyl, pyrrolidinyl, azetidinyl, piperidinyl, morpholinyl or piperazinyl, each of which is optionally substituted with 1-2 R⁷;

R⁶ is hydrogen or C₁-C₈ alkyl;

R⁷ is C₁-C₈ alkyl, C₁-C₈ alkoxy, halo, halo C₁-C₈ alkyl, C₁-C₈ alkylamino, di C₁-C₈ alkylamino, oxo, —C(O)NR^{b}R^{b′} or —C(O)R^{e}, each of which is optionally substituted with R¹²;

R⁹ is C₁-C₈ alkyl, C₁-C₈ alkoxy, oxazolyl, thiazolyl C₃-C₈ cycloalkyl, halo, cyano or —C(O)NR^{b}R^{b′}, each of which is optionally substituted with 1-2 R¹²;

R¹² is C₁-C₈ alkoxy or —C(O)NR^{b}R^{b′} and

each R^{a}, R^{b}, R^{b′}, R^{c}, R^{c′}, R^{d}, R^{d′}, R^{e} and R^{f} is independently hydrogen or C₁-C₈ alkyl.

Item 53. The compound according to Item 25, wherein

m is 0;

R⁷ is C₁-C₈ alkyl, halo, haloalkyl, —CN, —C(O)NR^{b}R^{b′} or —OR^{d}, each of which is optionally substituted with 1-3 R¹², wherein two R⁷ may, together with the ring to which they are attached, form benzoxazolyl;

n is 0, 1 or 2

R⁹ is —CH₃ or —CH₂CH₃;

R¹² is C₁-C₈ alkyl or halo;

each R^{a}, R^{b}, R^{b′}, R^{c}, R^{c′}, R^{d}, R^{d′}, R^{e} and R^{f} is independently hydrogen or C₁-C₈ alkyl.

Item 54. The compound according to Item 30, wherein
m is 1, 2 or 3;

R² is halo, —OR^{d}, piperazinyl, phenyl, pyridyl, pyrimidinyl or benzodioxolyl, wherein the phenyl is optionally substituted with 1-2 R⁹;

R⁴ is hydrogen or C₁-C₈ alkyl;

R⁷ is C₁-C₈ alkyl, halo, —NO₂, —NR^{c}C(O)R^{c′} or —OR^{d};

R⁹ is C₁-C₈ alkyl, halo, —CN, —NO₂, —C(O)NR^{b}R^{b′}, —NR^{c}C(O)R^{c′} or —NR^{b}R^{b′};

and

each R^{a}, R^{b}, R^{b′}, R^{c}, R^{c′}, R^{d}, R^{d′}, R^{e} and R^{f} is independently hydrogen or C₁-C₈ alkyl,
Item 55. The compound according to Item 45, wherein

R⁹ is C₁-C₈ alkyl, halo, —CN or —OR^{d};

t is 1 to 4, wherein two R⁹ may be taken together with the ring atoms to which they are attached to form an optionally substituted indolyl, indazolyl or benzothienyl;

R¹¹ is C₁-C₈ alkyl; and

R^{d} is C₁-C₈ alkyl.

Item 56. The compound according to Item 15, wherein

R¹ is C₁-C₈ alkyl;

R⁴ is hydrogen, halo, haloalkyl, haloalkoxy or —OR^{d};

m is 1;

R⁹ is halo, —CN, —C(O)NR^{b}R^{b′} or —OR^{d};

n is 1 or 2; and

each R^{b}, R^{b′} and R^{d} is independently C₁-C₈ alkyl.

Item 57. The compound according to Item 20, wherein

R¹ is C₁-C₈ alkyl;

R⁴ is C₁-C₈ alkyl or halo;

m is 1;

R⁹ is C₁-C₈ alkyl, halo, haloalkyl, —CN or —OR^{d}, each of which is optionally substituted with 1 R¹², wherein two R⁹ may, together with the ring atoms to which they are attached, form indazolyl or benzothienyl;

R¹² is C₁-C₈ alkyl; and

R^{d} is C₁-C₈ alkyl.

Item 58. The compound according to Item 37, wherein

m is 1;

n is 1 or 2;

R⁴ is hydrogen, or —OR^{d};

R⁹ is halo, —CN or —OR^{d}; or

each R^{d} is C₁-C₈ alkyl.

Item 59. The compound according to Item 1, which is

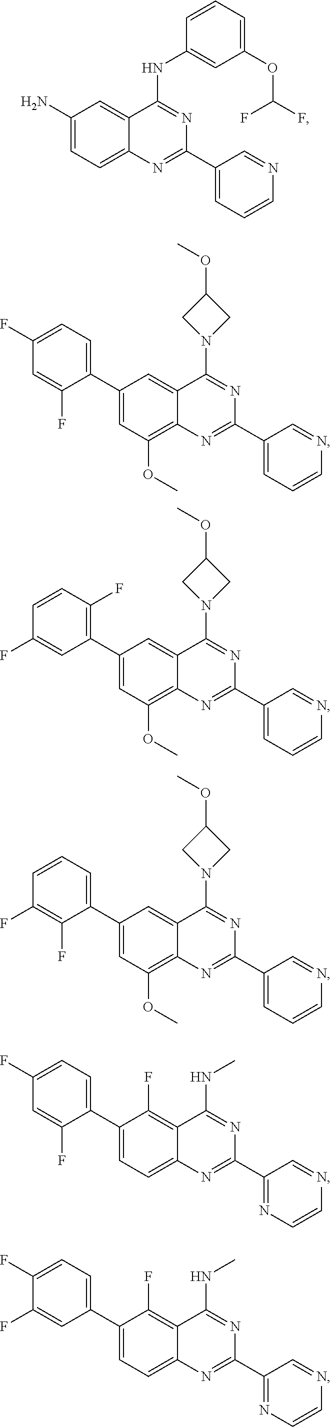

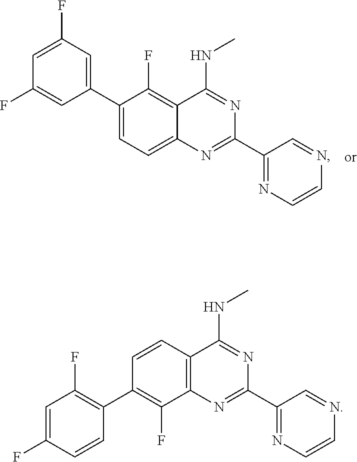 [SMILES: CNc1nc(-c2cnccn2)nc2ccc(-c3cc(F)cc(F)c3)c(F)c12 ; CNc1nc(-c2cnccn2)nc2c(F)c(-c3ccc(F)cc3F)ccc12]

Item 60. A pharmaceutical composition comprising the compound or a salt thereof according to any one of Items 1 to 59 as an active ingredient and a pharmaceutically acceptable carrier.

Item 61. The pharmaceutical composition according to Item 60 for preventing or treating central nervous system diseases.

Item 62. The pharmaceutical composition according to Item 61 for treating or preventing central nervous system disorders selected from the group consisting of schizophrenia; refractory, intractable or chronic schizophrenia; emotional disturbance; psychotic disorder; mood disorder; bipolar I type disorder; bipolar II type disorder; depression; endogenous depression; major depression; melancholy and refractory depression; dysthymic disorder; cyclothymic disorder; panic attack; panic disorder; agoraphobia; social phobia; obsessive-compulsive disorder; post-traumatic stress disorder; generalized anxiety disorder; acute stress disorder; hysteria; somatization disorder; conversion disorder; pain disorder; hypochondriasis; factitious disorder; dissociative disorder; sexual dysfunction; sexual desire disorder; sexual arousal disorder; erectile dysfunction; anorexia nervosa; bulimia nervosa; sleep disorder; adjustment disorder; alcohol abuse; alcohol intoxication; drug addiction; stimulant intoxication; narcotism; anhedonia; iatrogenic anhedonia; anhedonia of a psychic or mental cause; anhedonia associated with depression; anhedonia associated with schizophrenia; delirium; cognitive impairment; cognitive impairment associated with Alzheimer's disease, Parkinson's disease and other neurodegenerative diseases; cognitive impairment caused by Alzheimer's disease; Parkinson's disease and associated neurodegenerative diseases; cognitive impairment of schizophrenia; cognitive impairment caused by refractory, intractable or chronic schizophrenia; vomiting; motion sickness; obesity; migraine; pain (ache); mental retardation; autism disorder (autism); Tourette's disorder; tic disorder; attention-deficit/hyperactivity disorder; conduct disorder; and Down's syndrome.
Item 63. A process for producing a pharmaceutical composition comprising mixing a compound or a salt thereof according to any one of Items 1 to 59 with a pharmaceutically acceptable carrier.
Item 64. Use of a compound or a salt thereof according to any one of Items 1 to 59 as a drug.
Item 65. Use of the compound or a salt thereof according to any one of Items 1 to 59 as a STEP inhibitor.
Item 66. A method of treating a disorder that would benefit by the modulation of STEP (e.g., by activation of inhibition of STEP) in a subject, the method comprising administering to a compound or a salt thereof according to any one of Items 1 to 59.
Item 67. The method of Item 66, wherein the disorder is schizophrenia.
Item 68. The method of Item 66, wherein the disorder is cognitive deficit.
Item 69. The method of Item 66, wherein the compound or a salt thereof is administered in combination with an additional therapeutic agent.
Item 70. The method of Item 66, wherein the additional therapeutic agent is an atypical antipsychotic.
Item 71. The method of Item 66, wherein the additional therapeutic agent is selected from the group consisting of aripiprazole, clozapine, ziprasidone, risperidone, quetiapine, olanzapine, amisulpride, asenapine, iloperidone, melperone, paliperidone, perospirone, sertindole and sulpiride.
Item 72. The method of Item 66, wherein the additional therapeutic agent is a typical antipsychotic.
Item 73. The method of Item 66, wherein the additional therapeutic agent is selected from the group consisting of haloperidol, molindone, loxapine, thioridazine, molindone, thiothixene, pimozide, fluphenazine, trifluoperazine, mesoridazine, chlorprothixene, chlorpromazine, perphenazine, triflupromazine and zuclopenthixol.
Item 74. A kit comprising a composition comprising a compound or a salt thereof according to any one of Items 1 to 59 and an acceptable carrier.
Item 75. A kit comprising a pharmaceutical composition comprising a compound or a salt thereof according to any one of Items 1 to 59 and a pharmaceutically acceptable carrier.

In one aspect, a compound of formula (I):

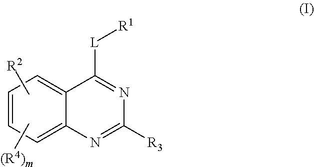

or a salt thereof,

wherein:

m is 0 or 1;

L is a direct bond or NR⁶;

R¹ is hydrogen, C₁-C₈ alkyl, halo C₁-C₈ alkyl, C₁-C₈ alkoxy C₁-C₈ alkyl, hydroxy C₁-C₈ alkyl, amino C₁-C₈ alkyl, oxazolyl, thiazolyl, isoxazolyl, pyridyl, pyrrolopyridyl, oxadiazolyl C₁-C₈ alkyl, pyridyl C₁-C₈ alkyl, oxazolyl C₁-C₈ alkyl, phenyl C₁-C₈ alkyl, —C(O)R^{e}, pyrrolidinyl, azetidinyl, indolinyl, piperidinyl, morpholinyl, piperazinyl, phenyl, C₃-C₈ cycloalkyl, C₃-C₈ cycloalkyl C₁-C₈ alkyl, benzoxazolyl, each of which is optionally substituted with 1-2 R⁷;

R² is C₁-C₈ alkoxy, benzodioxolyl, piperazinyl, halo, phenyl, tetrahydronaphtyl, furyl, oxazolyl, thiazolyl, thiadiazolyl, pyridyl, pyrimidinyl, indolyl, indazolyl, dihydroindazolyl, tetrahydroisoquinolinyl, tetrahydroquinolinyl, dihydrobenzoimidazolyl, dihydrobenzoxazolyl, benzothiazolyl, dihydrobenzothiazolyl, benzothienyl, dihydroisoquinolinyl, isoquinolinyl, benzofuryl, dihydrobenzofuryl, benzodioxolyl, dihydrobenzoxazinyl, dihydrobenzodioxepinyl, tetrahydrobenzoxazepinyl, isoindolinyl, indolinyl, thienyl or dihydrobenzodioxinyl, each of which is optionally substituted with 1-3 R⁹;

R³ is pyridyl, pyrimidinyl, pyrazinyl or pyridazinyl, each of which is optionally substituted with C₁-C₈ alkyl, C₁-C₈ alkoxy, halo, halo C₁-C₈ alkyl, halo C₁-C₈ alkoxy, cyano or —OR^{d};

R⁴ is hydrogen, C₁-C₈ alkyl, C₁-C₈ alkoxy, halo, halo C₁-C₈ alkyl or halo C₁-C₈ alkoxy, each of which is optionally substituted with R¹⁰;

R⁶ is hydrogen or C₁-C₈ alkyl;

R⁷ is C₁-C₈ alkyl, C₁-C₈ alkoxy, pyrazolyl, pyridyl, C₃-C₈ cycloalkyl, halo, halo C₁-C₈ alkyl, halo C₁-C₈ alkoxy, C₁-C₈ alkylamino, di C₁-C₈ alkylamino, di C₁-C₈ alkylamino C₁-C₈ alkyl, cyano, oxo, nitro, —C(O)NR^{b}R^{b′}, —NR^{c}C(O)R^{c′} or —C(O)R^{e}, each of which is optionally substituted with R¹²;

R⁹ is C₁-C₈ alkyl, C₁-C₈ alkoxy, phenyl, pyrazolyl, dihydrobenzoxazolyl, oxazolyl, tetrazolyl, imidazolyl, thiazolyl, C₃-C₈ cycloalkyl, oxetanyl, pyrrolidinyl, morpholinyl, halo, halo C₁-C₈ alkyl, halo C₁-C₈ alkoxy, hydroxy C₁-C₈ alkyl, oxo, cyano, nitro, —C(O)OR^{a}, —C(O)NR^{b}R^{b′}, —NR^{c}C(O)R^{c′}, —NR^{b}R^{b′}, —OR^{d}, —SR^{d′}, —C(O)R^{e} or —S(O)_{q}R^{f}, each of which is optionally substituted with 1-2 R¹²;

R¹⁰ is C₁-C₈ alkoxy, C₂-C₈ alkenyl, C₃-C₈ cycloalkyl, furyl, thienyl, pyrazolyl, morpholinyl, piperazinyl, pyrrolidinyl, piperidinyl, tetrahydropyranyl, cyano, —C(O)NR^{b}R^{b′}, —NR^{c}C(O)R^{c′}, —NR^{b}R^{b′} or —S(O)_{q}R^{f}, each of which is optionally substituted with R¹²;

R¹² is C₁-C₈ alkyl, C₁-C₈ alkoxy, halo, halo C₁-C₈ alkyl, silyl C₁-C₈ alkoxy, silyl C₁-C₈ alkoxy C₁-C₈ alkyl, oxo, thioxo, cyano, nitro, —C(O)OR^{a}, —C(O)NR^{b}R^{b′}, —NR^{c}C(O)R^{c′}, —NR^{b}R^{b′}, —OR^{d} or —C(O)R^{e}□

each R^{a}, R^{b}, R^{b′}, R^{c}, R^{c′}, R^{d}, R^{d′}, R^{e} and R^{f} is independently hydrogen, amino, C₁-C₈ alkyl, C₁-C₈ alkoxy, C₂-C₈ alkenyl, C₁-C₈ alkoxy C₁-C₈ alkyl, C₃-C₈ cycloalkyl, tetrahydropyranyl, morpholinyl, thiadiazolyl or thiazolyl; and

q is 1 or 2.

In an embodiment, if R³ is

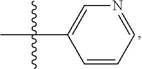 [SMILES: *c1cccnc1]

L is NR⁶, R^{f} is benzyl, R⁶ is hydrogen, and R⁴ is hydrogen, then R² is not halo or methoxy. In another embodiment, if R³ is

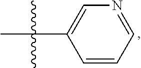 [SMILES: *c1cccnc1]

L is NR⁶, R¹ is phenyl, R⁶ is methyl, and R⁴ is hydrogen, then R² is not halo. In another embodiment, if R³ is

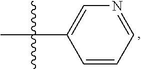 [SMILES: *c1cccnc1]

L is NR⁶, R¹ is para-trifluoromethyl-phenyl R⁶ is hydrogen, and R⁴ is hydrogen, then R² is not

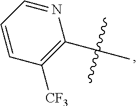 [SMILES: *c1ncccc1C(F)(F)F]

In another embodiment, if R³ is

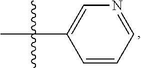 [SMILES: *c1cccnc1]

L is NR⁶, R¹ is indolinyl, R⁶ is hydrogen, and R⁴ is hydrogen, then R² is not chloro. In another embodiment, if R³ is

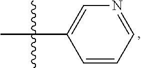 [SMILES: *c1cccnc1]

L is NR⁶, R¹ is dimethylaminomethyl, R⁶ is hydrogen, and R⁴ is methoxy, then R² is not methoxy. In another embodiment, the compound is not a compound shown in Table X.

In an embodiment, R¹ is C₃-C₈ cycloalkyl, oxazolyl, thiazolyl, isoxazolyl, pyridyl, pyrrolopyridyl, indolinyl, phenyl or benzoxazolyl, each of which is optionally substituted with 1-2 R⁷; R² is C₁-C₈ alkoxy, piperazinyl, halo or pyrimidinyl, each of which is optionally substituted with 1-3 R⁹; R³ is pyridyl (e.g, 3-pyridyl); R⁴ is hydrogen; R⁶ is hydrogen; R⁷ is C₁-C₈ alkyl, C₁-C₈ alkoxy, halo, halo C₁-C alkyl, cyano, nitro or —C(O)NR^{b}R^{b′} or —NR^{c}C(O)R^{c′}; R⁹ is C₁-C₈ alkyl, C₁-C₈ alkoxy, halo, cyano, nitro, —C(O)NR^{b}R^{b′} or —NR^{c}C(O)R^{c′}, —NR^{b}R^{b′}; each R^{a}, R^{b}, R^{b′}, R^{c}, and R^{c′} is independently hydrogen, C₁-C₈ alkyl or

C₁-C₈ alkoxy; and q is 1 or 2.

In an embodiment, R¹ is C₁-C₈ alkyl, phenyl or pyridyl C₁-C₈ alkyl, each of which is optionally substituted with 1-2 R⁷; R² is C₁-C₈ alkoxy or phenyl, each of which is optionally substituted with 1-3 R⁹; R³ is pyrimidinyl, pyrazinyl or pyridazinyl; R⁴ is hydrogen or C₁-C₈ alkoxy; R⁶ is hydrogen; R⁷ is C₁-C₈ alkyl or —C(O)NH₂; R⁹ is halo; and q is 1 or 2.

In an embodiment, m is 0 or 1; R¹ is hydrogen, C₁-C₈ alkyl, halo C₁-C₈ alkyl, C₁-C₈ alkoxy C₁-C₈ alkyl, hydroxyl C₁-C₈ alkyl, amino C₁-C₈ alkyl, oxazolyl, thiazolyl, isoxazolyl, pyridyl, pyrrolopyridyl, oxadiazolyl C₁-C₈ alkyl, pyridyl C₁-C₈ alkyl, oxazolyl C₁-C₈ alkyl, phenyl C₁-C₈ alkyl, —C(O)R^{e}, C₃-C₈ cycloalkyl, C₃-C₈ cycloalkyl C₁-C₈ alkyl, pyrrolidinyl, azetidinyl, indolinyl, piperidinyl, morpholinyl or piperazinyl, each of which is optionally substituted with 1-2 R⁷;

R² is phenyl, tetrahydronaphthyl, furyl, oxazolyl, thiazolyl, thiadiazolyl, pyridyl, pyrimidinyl, indolyl, indazolyl, dihydroindazolyl, tetrahydroisoquinolinyl, tetrahydroquinolinyl, dihydrobenzoimidazolyl, dihydrobenzoxazolyl, benzothiazolyl, dihydrobenzothiazolyl, benzothienyl, dihydroisoquinolinyl, isoquinolinyl, benzofuryl, dihydrobenzofuryl, benzodioxolyl, dihydrobenzoxazinyl, dihydrobenzodioxepinyl, tetrahydrobenzoxazepinyl, isoindolinyl, indolinyl, thienyl or dihydrobenzodioxinyl, each of which is optionally substituted with 1-3 R⁹; R³ is pyridyl (e.g, 3-pyridyl), each of which is optionally substituted with C₁-C₈ alkyl, C₁-C₈ alkoxy, halo, halo C₁-C₈ alkyl, halo C₁-C₈ alkoxy, cyano or —OR^{d}; R⁴ is hydrogen, C₁-C₈ alkyl, C₁-C₈ alkoxy, halo, halo C₁-C₈ alkyl or halo C₁-C₈ alkoxy, each of which is optionally substituted with R¹⁰; R⁶ is hydrogen or C₁-C₈ alkyl; R⁷ is C₁-C₈ alkyl, C₁-C₈ alkoxy, pyrazolyl, pyridyl, C₃-C₈ cycloalkyl, halo,

halo C₁-C₈ alkyl, halo C₁-C₈ alkoxy, C₁-C₈ alkylamino, di C₁-C₈ alkylamino, di C₁-C₈ alkyl amino C₁-C₈ alkyl, oxo, nitro, —C(O)NR^{b}R^{b′}, —NR^{c}C(O)R^{c′} or —C(O)R^{e}, each of which is optionally substituted with R¹²; R⁹ is C₁-C₈ alkyl, C₁-C₈ alkoxy, phenyl, pyrazolyl, dihydrobenzoxazolyl,oxazolyl, tetrazolyl, imidazolyl, thiazolyl C₃-C₈ cycloalkyl, oxetanyl, pyrrolidinyl, morpholinyl, halo, halo C₁-C₈ alkyl, halo C₁-C₈ alkoxy, hydroxyl C₁-C₈ alkyl, oxo, cyano, nitro, —C(O)OR^{a}, —C(O)NR^{b}R^{b′}, —NR^{c}C(O)R^{c′}, —SR^{d′}, —C(O)R^{e} or —S(O)_{q}R^{f}, each of which is optionally substituted with 1-2 R¹²; is C₁-C₈ alkoxy, C₂-C₈ alkenyl, C₃-C₈ cycloalkyl, furyl, thienyl, pyrazolyl, morpholinyl, piperazinyl, pyrrolidinyl, piperidinyl, tetrahydropyranyl, cyano, —C(O)NR^{b}R^{b′}, —NR^{c}C(O)R^{c′}, —NR^{b}R^{b′} or —S(O)R^{f}, each of which is optionally substituted with R¹²; R¹² is C₁-C₈ alkyl, C₁-C₈ alkoxy, halo, halo C₁-C₈ alkyl, silyl C₁-C₈ alkoxy, silyl C₁-C₈ alkoxy C₁-C₈ alkyl, oxo, thioxo, cyano, nitro, —C(O)OR^{a}, —C(O)NR^{b}R^{b′}, —NR^{c}C(O)R^{c′}, —OR^{d} or —C(O)R^{e}; each R^{a}, R^{b}, R^{b′}, R^{c}, R^{c′}, R^{d}, R^{d′}, R^{e} and R^{f} is independently hydrogen, amino, C₁-C₈ alkyl, C₁-C₈ alkoxy, C₂-C₈ alkenyl, C₁-C₈ alkoxy C₁-C₈ alkyl, C₃-C₈ cycloalkyl, tetrahydropyranyl, morpholinyl, thiadiazolyl or thiazolyl; and q is 1 or 2.

In another embodiment, wherein R² is phenyl.

In another aspect, a compound of formula (II):

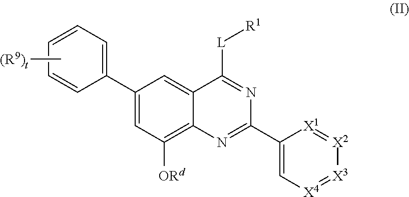

or a salt thereof,
wherein:

L is a direct bond or NR⁶; one or two of X¹, X², X³, and X⁴ are N and the others are CH, R¹ is hydrogen, C₁-C₈ alkyl, C₂-C₈ alkenyl, C₂-C₈ alkynyl, alkoxyalkyl, hydroxyalkyl, heteroaryl, heteroarylalkyl, arylalkyl, —C(Y)R^{e}, cyclyl,cyclylalkyl or heterocyclyl, each of which is optionally substituted with 1-3 R⁷; R⁶ is hydrogen, C₁-C₈ alkyl, C₂-C₈ alkenyl, C₂-C₈ alkynyl, cyclyl or heterocyclyl, each of which is optionally substituted with 1-3 R¹¹; R⁷ is C₁-C₈ alkyl, C₂-C₈ alkenyl, C₂-C₈ alkynyl, aryl, heteroaryl, cyclyl, heterocyclyl, arylalkyl, heteroarylalkyl, cyclylalkyl, heterocyclylalkyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹²; wherein two R⁷ may be taken together with the atoms to which they are attached to form an optionally substituted cyclyl, heterocyclyl, aryl or heteroaryl ring; R⁹ is C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹²; t is 1 to 4, wherein two R⁹ may be taken together with the atoms to which they are attached to form an optionally substituted cyclyl, heterocyclyl, aryl or heteroaryl ring; each R¹¹ and R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹³; R¹³ is independently C₁-C₈ alkyl, haloalkyl,halo, heterocyclyl, cyclyl, oxo or —C(Y)NR^{b}R^{b′}; Y is independently O or S; q is 1 or 2; and each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.

In an embodiment, R¹ is C₁-C₈ alkyl, halo C₁-C₈ alkyl, C₁-C₈ alkoxy C₁-C₈ alkyl, hydroxyl C₁-C₈ alkyl, amino C₁-C₈ alkyl, oxadiazolyl C₁-C₈ alkyl, oxazolyl C₁-C₈ alkyl, —C(O)R^{e}, C₃-C₈ cycloalkyl, pyrrolidinyl, azetidinyl, piperidinyl, morpholinyl or piperazinyl, each of which is optionally substituted with 1-2 R⁷; R⁶ is hydrogen or C₁-C₈ alkyl; R⁷ is C₁-C₈ alkyl, C₁-C₈ alkoxy, halo, halo C₁-C₈ alkyl, C₁-C₈ alkylamino, di C₁-C₈ alkylamino, oxo, —C(O)NR^{b}R^{b′} or —C(O)R^{e}, each of which is optionally substituted with R¹²; R⁹ is C₁-C₈ alkyl, C₁-C₈ alkoxy, oxazolyl, thiazolyl C₃-C₈ cycloalkyl, halo, cyano or —C(O)NR^{b}R^{b′}, each of which is optionally substituted with 1-2 R¹²; R¹² is C₁-C₈ alkoxy or —C(O)NR^{b}R^{b′} and each R^{a}, R^{b}, R^{b′}, R^{c}, R^{c′}, R^{d}, R^{d′}, R^{e} and R^{f} is independently hydrogen or C₁-C₈ alkyl. In another embodiment, if X₂ is N and X₁, X₃, X₄ are CH,

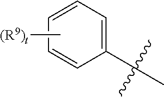

is not

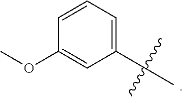 [SMILES: *c1cccc(OC)c1]

In another embodiment, the compound is not in Table X. In another embodiment, X₂ is N, and X₁, X₃, and X₄ are CH. In another embodiment, X₁ and X₃ are N, and X₂ and X₄ are CH. In another embodiment, R^{d} is methyl. In another embodiment, R⁹ is fluoro.

In another aspect, a compound of formula (III):

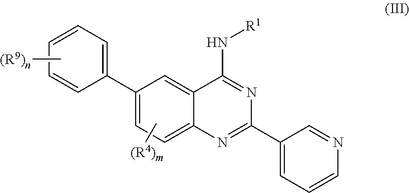

wherein:

R¹ is hydrogen, C₁-C₈ alkyl, halo C₁-C₈ alkyl, C₁-C₈ alkoxy C₁-C₈ alkyl, hydroxy C₁-C₈ alkyl, amino C₁-C₈ alkyl, oxazolyl, thiazolyl, isoxazolyl, pyridyl, pyrrolopyridyl, oxadiazolyl C₁-C₈ alkyl, pyridyl C₁-C₈ alkyl, oxazolyl C₁-C₈ alkyl, phenyl C₁-C₈ alkyl, —C(O)R^{e}, pyrrolidinyl, azetidinyl, indolinyl, piperidinyl, morpholinyl, piperazinyl, phenyl, C₃-C₈ cycloalkyl, C₃-C₈ cycloalkyl C₁-C₈ alkyl, benzoxazolyl, each of which is optionally substituted with 1-2 R⁷; each R⁴ is independently hydrogen, C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹⁰; m is 1 or 2; each R⁷, R⁹, or R¹⁰ is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹² wherein two R⁹ may, together with the ring atoms to which they are attached, form a five or six-membered aryl, heteroaryl, cyclic, or heterocyclic; n is 1, 2, or 3; each R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹³; each R¹³ is independently C₁-C₈ alkyl, haloalkyl, halo, heterocyclyl, cyclyl, oxo or —C(Y)NR^{b}R^{b′}; Y is independently O or S; q is 1 or 2; and each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.

In an embodiment, R¹ is C₁-C₈ alkyl; R⁴ is hydrogen, halo, haloalkyl, haloalkoxy or —OR^{d}; m is 1; R⁹ is halo, —CN, —C(O)NR^{b}R^{b′} or —OR^{d}; n is 1 or 2; and each R^{b}, R^{b′} and R^{d} is independently C₁-C₈ alkyl. In another embodiment, if R¹ is methyl or phenyl and R⁴ is methyl, then R⁹ is not fluoro, cyano, or methoxy. In another embodiment, if formula (III) is formula (III′):

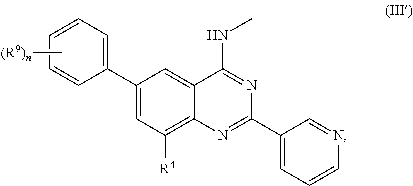

and R⁴ is fluoro or methoxy, then R⁹ is not fluoro or methoxy.

In another embodiment, if t formula (III) is formula (III′):

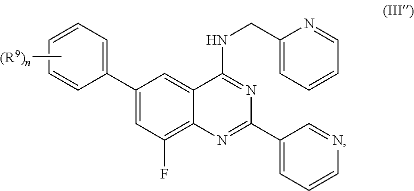

then R⁹ is not fluoro.

In another embodiment, the compound is not

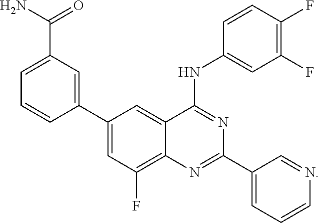 [SMILES: NC(=O)c1cccc(-c2cc(F)c3nc(-c4cccnc4)nc(Nc4ccc(F)c(F)c4)c3c2)c1]

In another embodiment, the compound is not in Table X. In another embodiment, R¹ is C₁-C₈ alkyl. In another embodiment, wherein R⁹ is halo.

In another aspect, a compound of formula (IV):

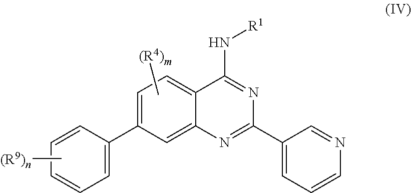

wherein:

R¹ is hydrogen, C₁-C₈ alkyl, halo C₁-C₈ alkyl, C₁-C₈ alkoxy C₁-C₈ alkyl, hydroxy C₁-C₈ alkyl, amino C₁-C₈ alkyl, oxazolyl, thiazolyl, isoxazolyl, pyridyl, pyrrolopyridyl, oxadiazolyl C₁-C₈ alkyl, pyridyl C₁-C₈ alkyl, oxazolyl C₁-C₈ alkyl, phenyl C₁-C₈ alkyl, —C(O)R^{e}, pyrrolidinyl, azetidinyl, indolinyl, piperidinyl, morpholinyl, piperazinyl, phenyl, C₃-C₈ cycloalkyl, C₃-C₈ cycloalkyl C₁-C₈ alkyl, benzoxazolyl, each of which is optionally substituted with 1-2 R⁷;
each R⁴ is independently hydrogen, C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹⁰; m is 1 or 2; each R⁷, R⁹, or R¹⁰ is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹², wherein two R⁹ may, together with the ring atoms to which they are attached, form a five or six-membered aryl, heteroaryl, cyclic, or heterocyclic; n is 1, 2, or 3; each R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹³; each R¹³ is independently C₁-C₈ alkyl, haloalkyl, halo, heterocyclyl, cyclyl, oxo or —C(Y)NR^{b}R^{b′}; Y is independently O or S; q is 1 or 2; and each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.

In an embodiment, R¹ is C₁-C₈ alkyl; R⁴ is C₁-C₈ alkyl or halo; m is 1; R⁹ is C₁-C₈ alkyl, halo, haloalkyl, —CN or —OR^{d}, each of which is optionally substituted with 1 R¹², wherein two R⁹ may, together with the ring atoms to which they are attached, form indazolyl or benzothienyl; R¹² is C₁-C₈ alkyl; and R^{d} is C₁-C₈ alkyl. In another embodiment, if R¹ is methyl and R⁴ is methyl, then R⁹ is not fluoro, cyano, or methoxy. In another embodiment, the compound is not in Table X. In another embodiment, R¹ is C₁-C₈ alkyl. In another embodiment, R⁴ is fluoro.

In another aspect, a compound of formula (V):

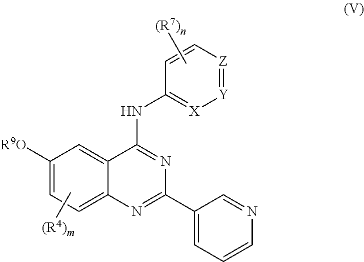

wherein:

one of X, Y, or Z is —N—, the rest being —CH— or —CR⁷—; each R⁴ is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹⁰; m is 0, 1, or 2; each R⁷ or R¹⁰ is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹², wherein two R⁷ may, together with the ring to which they are attached, form a five or six-membered aryl or heteroaryl; n is 0, 1, 2, or 3; R⁹ is —CH₃ or —CH₂CH₃; each R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹³; each R¹³ is independently C₁-C₈ alkyl, haloalkyl, halo, heterocyclyl, cyclyl, oxo or —C(Y)NR^{b}R^{b′}; Y is independently O or S; q is 1 or 2; and each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.

In an embodiment, m is 0; R⁷ is C₁-C₈ alkyl, halo, haloalkyl, —CN, —C(O)NR^{b}R^{b′} or —OR^{d}, each of which is optionally substituted with 1-3 R¹², wherein two R⁷ may, together with the ring to which they are attached, form benzoxazolyl; n is 0, 1 or 2; R⁹ is —CH₃ or —CH₂CH₃; R¹² is C₁-C₈ alkyl or halo; each R^{a}, R^{b}, R^{b′}, R^{c}, R^{c′}, R^{d}, R^{d′}, R^{e} and R^{f} is independently hydrogen or C₁-C₈ alkyl. In another embodiment, the compound is not

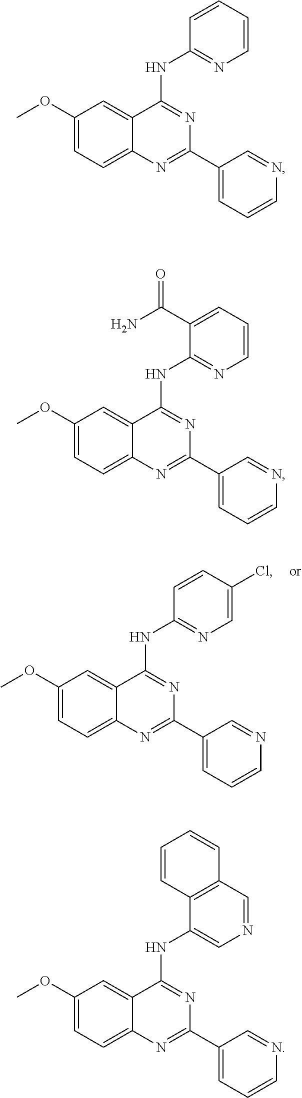 [SMILES: COc1ccc2nc(-c3cccnc3)nc(Nc3ccccn3)c2c1 ; COc1ccc2nc(-c3cccnc3)nc(Nc3ncccc3C(N)=O)c2c1 ; COc1ccc2nc(-c3cccnc3)nc(Nc3ccc(Cl)cn3)c2c1 ; COc1ccc2nc(-c3cccnc3)nc(Nc3cncc4ccccc34)c2c1]

In another embodiment, the compound is not in Table X. In another embodiment, R⁷ is halo. In another embodiment, m is 0.

In another aspect, a compound of formula (VI):

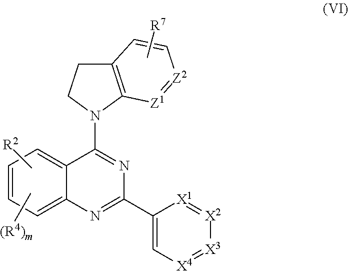

or a salt thereof,

wherein:

one or two of X¹, X², X³, and X⁴ are N and the others are CH; Z₁ and Z₂ are independently N or CH; m is 1, 2 or 3; R² is halo, —OR^{d}, aryl or heteroaryl, wherein the aryl or heteroaryl is optionally substituted with 1-5 R⁹; each R⁴ is independently hydrogen, C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹⁰; each R⁷, R⁹, and R¹⁰ is independently C₁-C₈ alkyl, C₂-C₈ alkenyl, C₂-C₈ alkynyl, aryl, heteroaryl, cyclyl, heterocyclyl, arylalkyl, heteroarylalkyl, cyclylalkyl, heterocyclylalkyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹²; each R¹² is independently C₁-C₈ alkyl, C₂-C₈ alkenyl, C₂-C₈ alkynyl, aryl, heteroaryl, cyclyl, heterocyclyl, arylalkyl, heteroarylalkyl, cyclylalkyl, heterocyclylalkyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f},each of which is optionally substituted with 1-3 R¹³; R¹³ is independently C₁-C₈ alkyl, haloalkyl,halo, heterocyclyl, cyclyl, oxo or —C(Y)NR^{b}R^{b′}; Y is independently O or S; q is 1 or 2; and each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.

In an embodiment, m is 1, 2 or 3; R² is halo, —OR^{d}, piperazinyl, phenyl, pyridyl, pyrimidinyl or benzodioxolyl, wherein the phenyl is optionally substituted with 1-2 R⁹; R⁴ is hydrogen or C₁-C₈ alkyl; R⁷ is C₁-C₈ alkyl, halo, —NO₂, —NR^{c}C(O)R^{c′} or —OR^{d}; R⁹ is C₁-C₈ alkyl, halo, —CN, —NO₂, —C(O)NR^{b}R^{b′}, —NR^{c}C(O)R^{c′} or —NR^{b}R^{b′}; and each R^{a}, R^{b}, R^{b′}, R^{c}, R^{c′}, R^{d}, R^{d′}, R^{e} and R^{f} is independently hydrogen or C₁-C₈ alkyl. In another embodiment, if Z₁ and Z₂ are both CH, R² is not —Cl or —OR^{d}. In another embodiment, the compound is not in Table X. In another embodiment, Z₁ is N. In another embodiment, R² is aryl. In another embodiment, R² is —Br or —I. In another embodiment,

X₂ is N, and X₁, X₃, and X₄ are CH.

In another aspect, a compound of formula (VII):

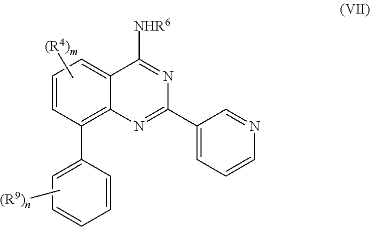

or a salt thereof,
wherein:

m is 1, 2 or 3; n is 1, 2, 3 or 4; each R⁴ is independently hydrogen, C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹⁰; R⁶ is hydrogen, C₁-C₈ alkyl, C₂-C₈ alkenyl, or C₂-C₈ alkynyl, each of which is optionally substituted with 1-3 R¹¹; each R⁹ and R¹⁰ is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹²; each R¹¹ and R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹³; R¹³ is independently C₁-C₈ alkyl, haloalkyl, halo, heterocyclyl, cyclyl, oxo or —C(Y)NR^{b}R^{b′}; Y is independently O or S; q is 1 or 2; and each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.

In an embodiment, m is 1; n is 1 or 2; R⁴ is hydrogen, or —OR^{d}; R⁹ is halo,—CN or —OR^{d}; each R^{d} is C₁-C₈ alkyl. In another embodiment, if R⁴ is hydrogen,

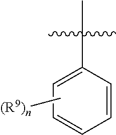

is not

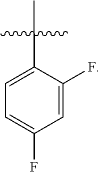 [SMILES: *c1ccc(F)cc1F]

In another embodiment, the compound is not in Table X. In another embodiment, R⁴ is —OCH₃. In another embodiment, R⁹ is —F.

In another aspect, a compound of formula (VIII):

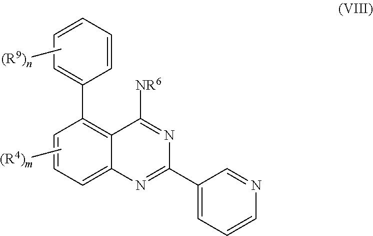

or a salt thereof,
wherein:

m is 1, 2 or 3; n is 1, 2, 3 or 4; each R⁴ is independently hydrogen, C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹⁰; R⁶ is hydrogen, C₁-C₈ alkyl, C₂-C₈ alkenyl, or C₂-C₈ alkynyl, each of which is optionally substituted with 1-3 R¹¹; each R⁹ and R¹⁰ is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹²; each R¹¹ and R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹³; R¹³ is independently C₁-C₈ alkyl, haloalkyl, halo, heterocyclyl, cyclyl, oxo or —C(Y)NR^{b}R^{b′}; Y is independently O or S; q is 1 or 2; and each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.

In an embodiment, the compound is not in Table X. In another embodiment, R⁹ is —F.

In another aspect, a compound of formula (IX) or (IX′):

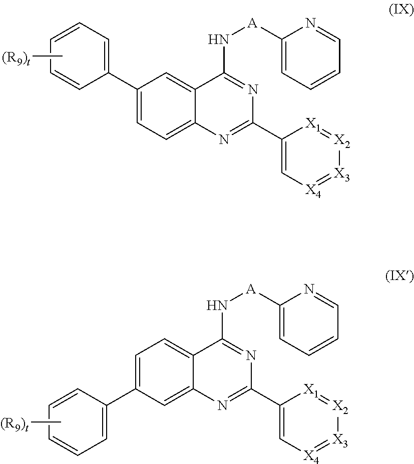

or a salt thereof,
wherein:

A is C₁-C₄ alkylene, optionally substituted with R¹¹; one or two of X¹, X², X³, and X⁴ are N and the others are CH, R⁹ is C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹²; t is 1 to 4, wherein two R⁹ may be taken together with the ring atoms to which they are attached to form an optionally substituted cyclyl, heterocyclyl, aryl or heteroaryl ring; each R¹¹ and R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹³; R¹³ is independently C₁-C₈ alkyl, haloalkyl, halo, heterocyclyl, cyclyl, oxo or —C(Y)NR^{b}R^{b′}; alternatively, R¹³ on R¹¹ may connect to the carbon atom of A to which R¹¹ bonds to form a C₃₋₆ cycloalkyl; Y is independently O or S; q is 1 or 2; and each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.

In an embodiment, R⁹ is C₁-C₈ alkyl, halo, —CN or —OR^{d}; t is 1 to 4, wherein two R⁹ may be taken together with the ring atoms to which they are attached to form an optionally substituted indolyl, indazolyl or benzothienyl; R¹¹ is C₁-C₈ alkyl; and R^{d} is C₁-C₈ alkyl. In an embodiment, if X₂ is N and X₁, X₃, X₄ are CH, R⁹ is not —F or —OR^{d}. In another embodiment, the compound is not in Table X. In another embodiment, A is —CH₂—.

In another embodiment, A is —C(CH₃)H—. In another embodiment, R⁹ is —F.

In another aspect, a compound disclosed herein.

### Aspects and Embodiments of Compounds of Formulas (I)-(IX′)

In another aspect, the invention features a composition comprising a compound of any of formulas (I)-(IX′) and an acceptable carrier.

In another aspect, the invention features a pharmaceutical composition comprising a compound of any of formulas (I)-(IX′) and a pharmaceutically acceptable carrier.

In another aspect, the invention features a kit comprising a composition comprising a compound of any of formulas (I)-(IX′) and an acceptable carrier.

In another aspect, the invention features a kit comprising a pharmaceutical composition comprising a compound of any of formulas (I)-(IX′) and a pharmaceutically acceptable carrier.

In another aspect, the invention features a dosage form comprising a composition comprising a compound of any of formulas (I)-(IX′) and an acceptable carrier.

In another aspect, the invention features a dosage form comprising a pharmaceutical composition comprising a compound of any of formulas (I)-(IX′) and a pharmaceutically acceptable carrier.

In another aspect, the invention features a method of treating a disorder that would benefit by the modulation of STEP (e.g., by activation or inhibition of STEP) in a subject, the method comprising administering to a subject in need thereof a compound of any of formulas (I)-(IX′).

In another aspect, the invention features a method of treating a disorder that would benefit by the inhibition of STEP, the method comprising administering to a subject in need thereof a compound of any of formulas (I)-(IX′). In some embodiments, the disorder is selected from schizophrenia, schizoaffective disorder, bipolar disorder, manic-depressive disorder, psychosis, mood and anxiety disorders, mania, drug or substance addiction, cognition disorders, learning disabilities, learning and memory disorders, aging and neurologic disorders associated with or linked with cognitive impairments; mild cognitive impairments (MCI), Alzheimer's disease, Alzheimer-related cognition disorders, Huntington's disease, Parkinson's disease, CADASIL syndrome (cerebral autosomal dominant arteriopathy with subcortical infarcts and leukoencephalopathy), amnesia, Wernicke-Korsakoff syndrome, Korsakoff syndrome, mild traumatic head injury (MBTI), traumatic head injury (TBI), fragile X syndrome, stroke, attention-deficit and hyperactivity disorder (ADHD), obsessive compulsive disorder (OCD), post-traumatic stress disorder (PTSD), loss of concentration, autism, cerebral palsy, encephalopathy, and narcolepsy. In some embodiments, the disorder affects learning and memory, neurogenesis, neuronal plasticity, pain perception, mood and anxiety, or neuroendocrine regulation. In some embodiments, the disorder is a cognitive deficit disorder. In some embodiments, the disorder involves pain perception or neuroendocrine regulation. In some embodiments, the disorder affects the central nervous system. In some embodiments the disorder is selected from the group consisting of schizophrenia; refractory, intractable or chronic schizophrenia; emotional disturbance; psychotic disorder; mood disorder; bipolar I type disorder; bipolar II type disorder; depression; endogenous depression; major depression; melancholy and refractory depression; dysthymic disorder; cyclothymic disorder; panic attack; panic disorder; agoraphobia; social phobia; obsessive-compulsive disorder; post-traumatic stress disorder; generalized anxiety disorder; acute stress disorder; hysteria; somatization disorder; conversion disorder; pain disorder; hypochondriasis; factitious disorder; dissociative disorder; sexual dysfunction; sexual desire disorder; sexual arousal disorder; erectile dysfunction; anorexia nervosa; bulimia nervosa; sleep disorder; adjustment disorder; alcohol abuse; alcohol intoxication; drug addiction; stimulant intoxication; narcotism; anhedonia; iatrogenic anhedonia; anhedonia of a psychic or mental cause; anhedonia associated with depression; anhedonia associated with schizophrenia; delirium; cognitive impairment; cognitive impairment associated with Alzheimer's disease, Parkinson's disease and other neurodegenerative diseases; cognitive impairment caused by Alzheimer's disease; Parkinson's disease and associated neurodegenerative diseases; cognitive impairment of schizophrenia; cognitive impairment caused by refractory, intractable or chronic schizophrenia; vomiting; motion sickness; obesity; migraine; pain (ache); mental retardation; autism disorder (autism); Tourette's disorder; tic disorder; attention-deficit/hyperactivity disorder; conduct disorder; and Down's syndrome.

In another aspect, the invention features a method of treating a condition that would benefit by the modulation of STEP (e.g., by activation or inhibition of STEP) in a subject, the method comprising administering to a subject in need thereof a compound of any of formulas (I)-(IX′). In some embodiments, the condition is selected from decreased neurogenesis, cell resilience, or neuronal plasticity due to normal aging, neurodegenerative disorders of the CNS; Alzheimer's disease, Huntington's disease, fragile X syndrome, amyotrophic lateral sclerosis/Lou Gehrig's disease, stroke, Parkinson's disease, parkinsonism, dementia, Pick disease, Corticobasal degeneration, Multiple system atrophy, Progressive supranuclear palsy, traumatic brain injury, head trauma, mild traumatic head injury (MBTI), traumatic head injury (TBI), encephalopathy, intoxication related to ethanol, alcoholism, fetal alcohol syndrome, drug addiction or drug abuse.

In some embodiments, a compound of any of formulas (I)-(IX′) is administered in combination with an additional therapeutic agent. In some embodiments, the additional therapeutic agent is an atypical antipsychotic. In some embodiments, the additional therapeutic agent is selected from the group consisting of aripiprazole, clozapine, ziprasidone, risperidone, quetiapine, olanzapine, amisulpride, asenapine, iloperidone, melperone, paliperidone, perospirone, sertindole and sulpiride. In some embodiments, the additional therapeutic agent is a typical antipsychotic. In some embodiments, the additional therapeutic agent is selected from the group consisting of haloperidol, molindone, loxapine, thioridazine, molindone, thiothixene, pimozide, fluphenazine, trifluoperazine, mesoridazine, chlorprothixene, chlorpromazine, perphenazine, triflupromazine and zuclopenthixol.

TABLE X

| | |
|---|---|
| 1 | 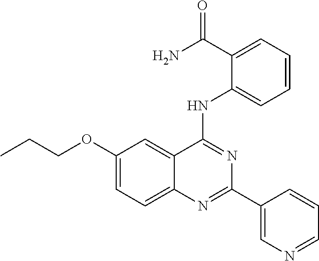 [SMILES: CCCOc1ccc2nc(-c3cccnc3)nc(Nc3ccccc3C(N)=O)c2c1] |
| 2 | 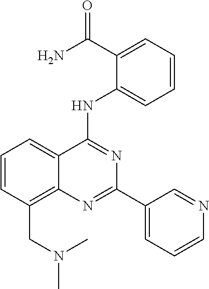 [SMILES: CN(C)Cc1cccc2c(Nc3ccccc3C(N)=O)nc(-c3cccnc3)nc12] |
| 3 | 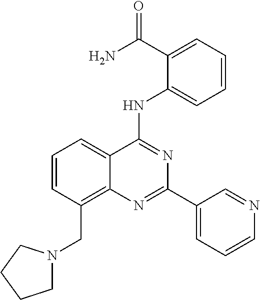 [SMILES: NC(=O)c1ccccc1Nc1nc(-c2cccnc2)nc2c(CN3CCCC3)cccc12] |
| 4 | 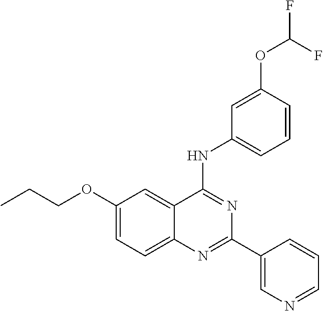 [SMILES: CCCOc1ccc2nc(-c3cccnc3)nc(Nc3cccc(OC(F)F)c3)c2c1] |
| 5 | 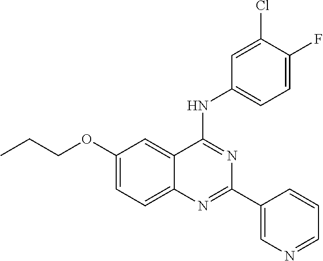 [SMILES: CCCOc1ccc2nc(-c3cccnc3)nc(Nc3ccc(F)c(Cl)c3)c2c1] |
| 6 | 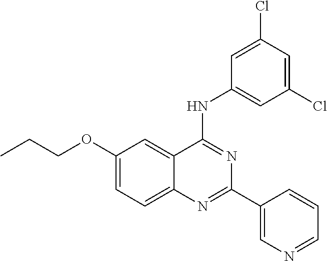 [SMILES: CCCOc1ccc2nc(-c3cccnc3)nc(Nc3cc(Cl)cc(Cl)c3)c2c1] |
| 7 | 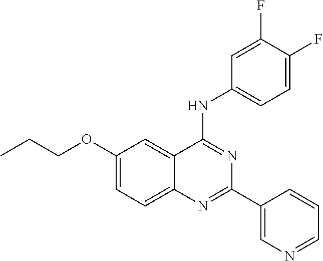 [SMILES: CCCOc1ccc2nc(-c3cccnc3)nc(Nc3ccc(F)c(F)c3)c2c1] |
| 8 | 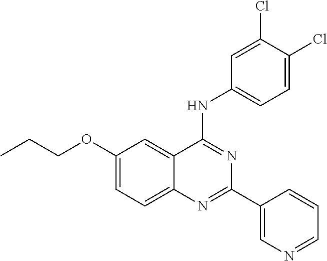 [SMILES: CCCOc1ccc2nc(-c3cccnc3)nc(Nc3ccc(Cl)c(Cl)c3)c2c1] |
| 9 | 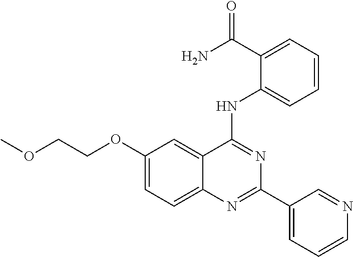 [SMILES: COCCOc1ccc2nc(-c3cccnc3)nc(Nc3ccccc3C(N)=O)c2c1] |
| 10 | 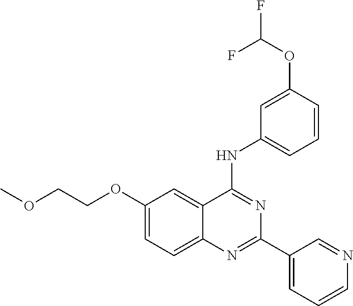 [SMILES: COCCOc1ccc2nc(-c3cccnc3)nc(Nc3cccc(OC(F)F)c3)c2c1] |
| 11 | 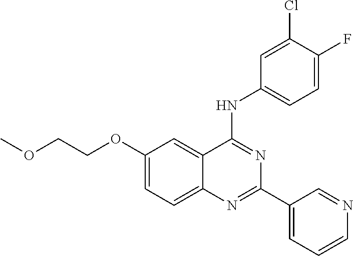 [SMILES: COCCOc1ccc2nc(-c3cccnc3)nc(Nc3ccc(F)c(Cl)c3)c2c1] |
| 12 | 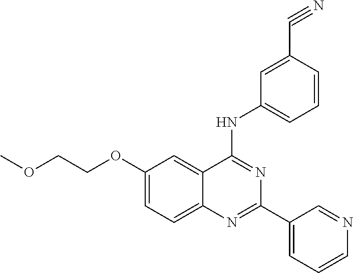 [SMILES: COCCOc1ccc2nc(-c3cccnc3)nc(Nc3cccc(C#N)c3)c2c1] |
| 13 | 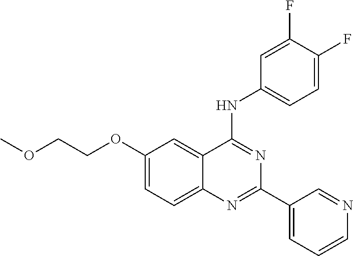 [SMILES: COCCOc1ccc2nc(-c3cccnc3)nc(Nc3ccc(F)c(F)c3)c2c1] |
| 14 | 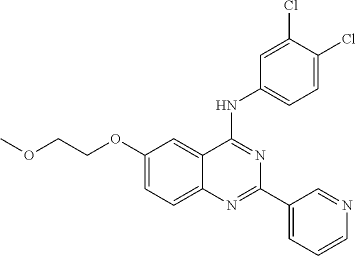 [SMILES: COCCOc1ccc2nc(-c3cccnc3)nc(Nc3ccc(Cl)c(Cl)c3)c2c1] |
| 15 | 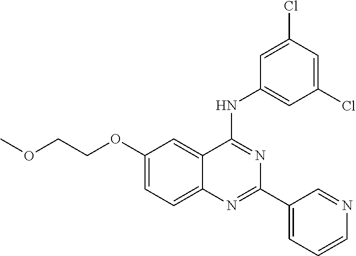 [SMILES: COCCOc1ccc2nc(-c3cccnc3)nc(Nc3cc(Cl)cc(Cl)c3)c2c1] |
| 16 | 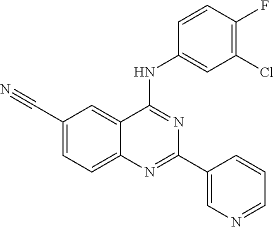 [SMILES: N#Cc1ccc2nc(-c3cccnc3)nc(Nc3ccc(F)c(Cl)c3)c2c1] |
| 17 | 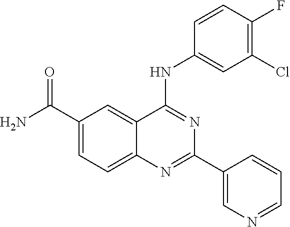 [SMILES: NC(=O)c1ccc2nc(-c3cccnc3)nc(Nc3ccc(F)c(Cl)c3)c2c1] |
| 18 | 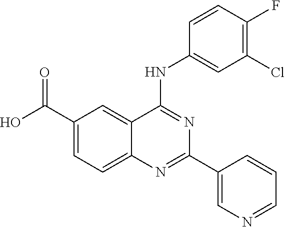 [SMILES: O=C(O)c1ccc2nc(-c3cccnc3)nc(Nc3ccc(F)c(Cl)c3)c2c1] |
| 19 | 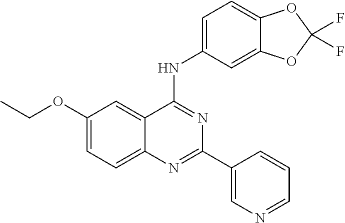 [SMILES: CCOc1ccc2nc(-c3cccnc3)nc(Nc3ccc4c(c3)OC(F)(F)O4)c2c1] |
| 20 | 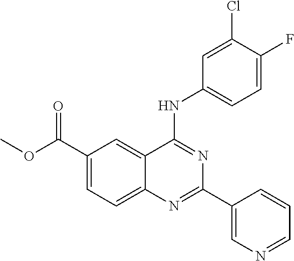 [SMILES: COC(=O)c1ccc2nc(-c3cccnc3)nc(Nc3ccc(F)c(Cl)c3)c2c1] |
| 21 | 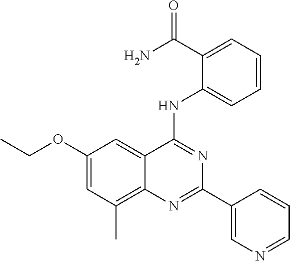 [SMILES: CCOc1cc(C)c2nc(-c3cccnc3)nc(Nc3ccccc3C(N)=O)c2c1] |
| 22 | 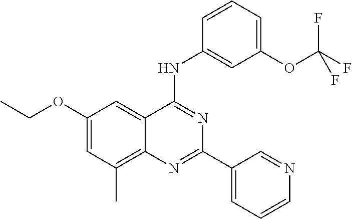 [SMILES: CCOc1cc(C)c2nc(-c3cccnc3)nc(Nc3cccc(OC(F)(F)F)c3)c2c1] |
| 23 | 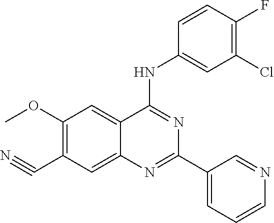 [SMILES: COc1cc2c(Nc3ccc(F)c(Cl)c3)nc(-c3cccnc3)nc2cc1C#N] |
| 24 | 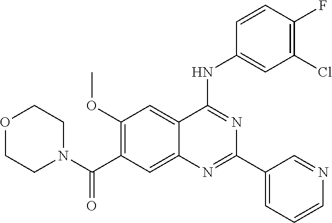 [SMILES: COc1cc2c(Nc3ccc(F)c(Cl)c3)nc(-c3cccnc3)nc2cc1C(=O)N1CCOCC1] |
| 25 | 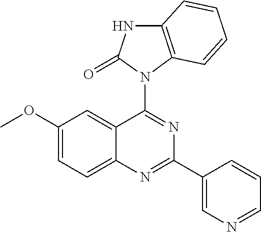 [SMILES: COc1ccc2nc(-c3cccnc3)nc(-n3c(=O)[nH]c4ccccc43)c2c1] |
| 26 | 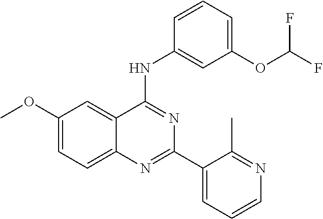 [SMILES: COc1ccc2nc(-c3cccnc3C)nc(Nc3cccc(OC(F)F)c3)c2c1] |
| 27 | 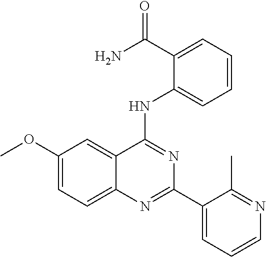 [SMILES: COc1ccc2nc(-c3cccnc3C)nc(Nc3ccccc3C(N)=O)c2c1] |
| 28 | 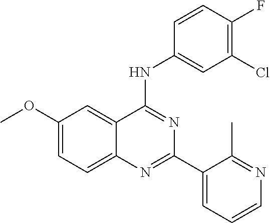 [SMILES: COc1ccc2nc(-c3cccnc3C)nc(Nc3ccc(F)c(Cl)c3)c2c1] |
| 29 | 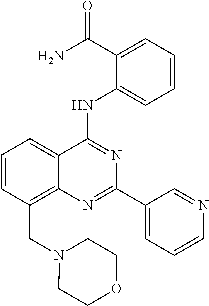 [SMILES: NC(=O)c1ccccc1Nc1nc(-c2cccnc2)nc2c(CN3CCOCC3)cccc12] |
| 30 | 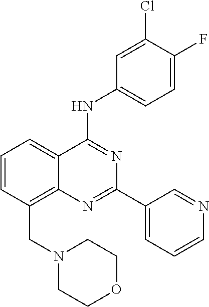 [SMILES: Fc1ccc(Nc2nc(-c3cccnc3)nc3c(CN4CCOCC4)cccc23)cc1Cl] |
| 31 | 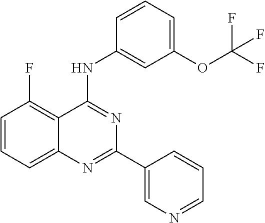 [SMILES: Fc1cccc2nc(-c3cccnc3)nc(Nc3cccc(OC(F)(F)F)c3)c12] |
| 32 | 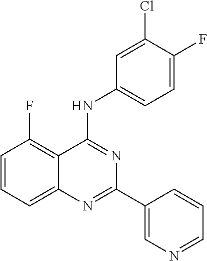 [SMILES: Fc1ccc(Nc2nc(-c3cccnc3)nc3cccc(F)c23)cc1Cl] |
| 33 | 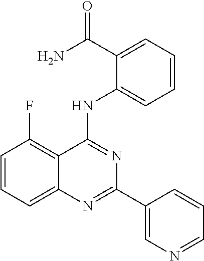 [SMILES: NC(=O)c1ccccc1Nc1nc(-c2cccnc2)nc2cccc(F)c12] |
| 34 | 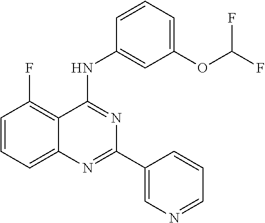 [SMILES: Fc1cccc2nc(-c3cccnc3)nc(Nc3cccc(OC(F)F)c3)c12] |
| 35 | 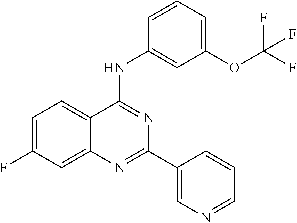 [SMILES: Fc1ccc2c(Nc3cccc(OC(F)(F)F)c3)nc(-c3cccnc3)nc2c1] |
| 36 | 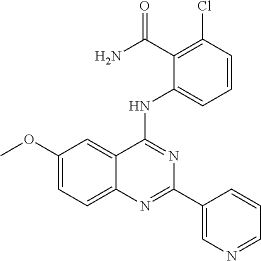 [SMILES: COc1ccc2nc(-c3cccnc3)nc(Nc3cccc(Cl)c3C(N)=O)c2c1] |
| 37 | 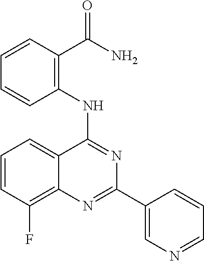 [SMILES: NC(=O)c1ccccc1Nc1nc(-c2cccnc2)nc2c(F)cccc12] |
| 38 | 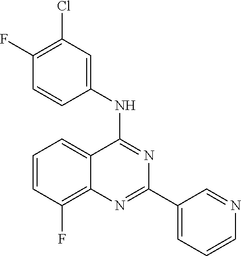 [SMILES: Fc1ccc(Nc2nc(-c3cccnc3)nc3c(F)cccc23)cc1Cl] |
| 39 | 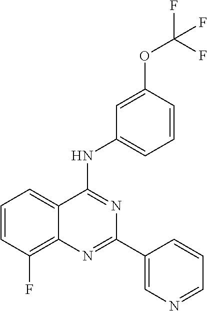 [SMILES: Fc1cccc2c(Nc3cccc(OC(F)(F)F)c3)nc(-c3cccnc3)nc12] |
| 40 | 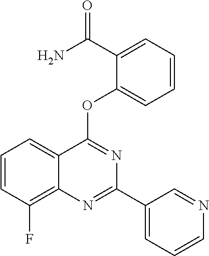 [SMILES: NC(=O)c1ccccc1Oc1nc(-c2cccnc2)nc2c(F)cccc12] |
| 41 | 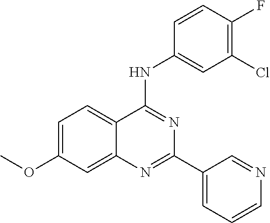 [SMILES: COc1ccc2c(Nc3ccc(F)c(Cl)c3)nc(-c3cccnc3)nc2c1] |
| 42 | 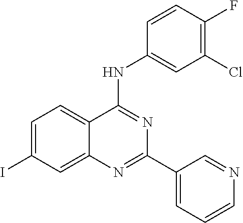 [SMILES: Fc1ccc(Nc2nc(-c3cccnc3)nc3cc(I)ccc23)cc1Cl] |
| 43 | 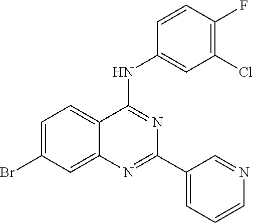 [SMILES: Fc1ccc(Nc2nc(-c3cccnc3)nc3cc(Br)ccc23)cc1Cl] |
| 44 | 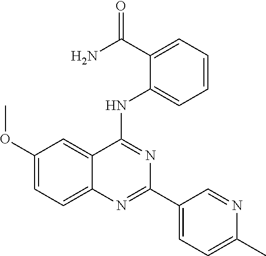 [SMILES: COc1ccc2nc(-c3ccc(C)nc3)nc(Nc3ccccc3C(N)=O)c2c1] |
| 45 | 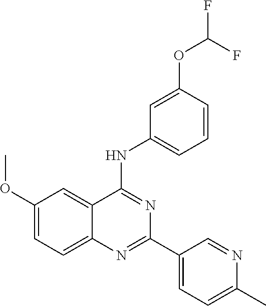 [SMILES: COc1ccc2nc(-c3ccc(C)nc3)nc(Nc3cccc(OC(F)F)c3)c2c1] |
| 46 | 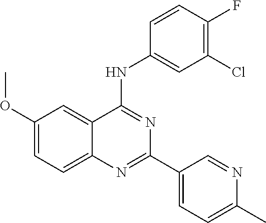 [SMILES: COc1ccc2nc(-c3ccc(C)nc3)nc(Nc3ccc(F)c(Cl)c3)c2c1] |
| 47 | 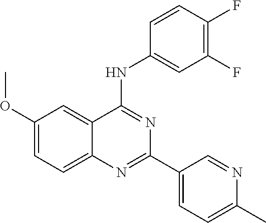 [SMILES: COc1ccc2nc(-c3ccc(C)nc3)nc(Nc3ccc(F)c(F)c3)c2c1] |
| 48 | 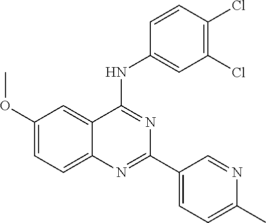 [SMILES: COc1ccc2nc(-c3ccc(C)nc3)nc(Nc3ccc(Cl)c(Cl)c3)c2c1] |
| 49 | 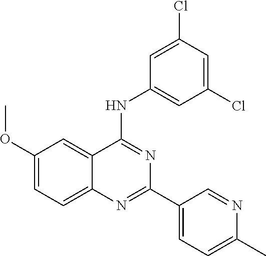 [SMILES: COc1ccc2nc(-c3ccc(C)nc3)nc(Nc3cc(Cl)cc(Cl)c3)c2c1] |
| 50 | 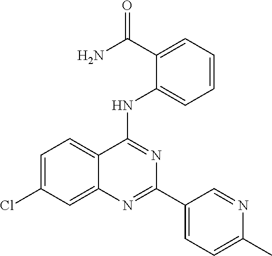 [SMILES: Cc1ccc(-c2nc(Nc3ccccc3C(N)=O)c3ccc(Cl)cc3n2)cn1] |
| 51 | 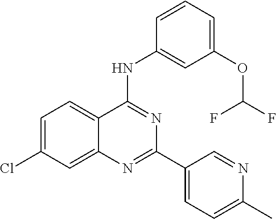 [SMILES: Cc1ccc(-c2nc(Nc3cccc(OC(F)F)c3)c3ccc(Cl)cc3n2)cn1] |
| 52 | 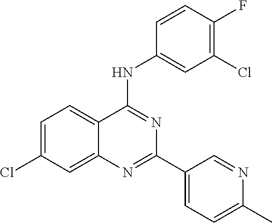 [SMILES: Cc1ccc(-c2nc(Nc3ccc(F)c(Cl)c3)c3ccc(Cl)cc3n2)cn1] |
| 53 | 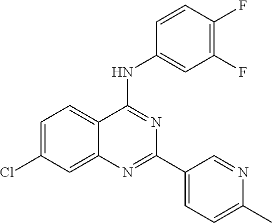 [SMILES: Cc1ccc(-c2nc(Nc3ccc(F)c(F)c3)c3ccc(Cl)cc3n2)cn1] |
| 54 | 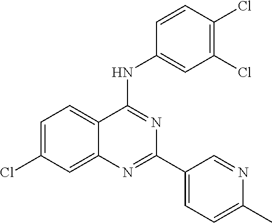 [SMILES: Cc1ccc(-c2nc(Nc3ccc(Cl)c(Cl)c3)c3ccc(Cl)cc3n2)cn1] |
| 55 | 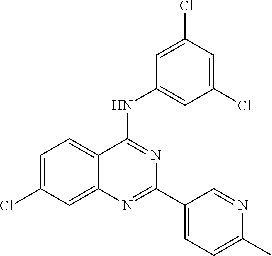 [SMILES: Cc1ccc(-c2nc(Nc3cc(Cl)cc(Cl)c3)c3ccc(Cl)cc3n2)cn1] |
| 56 | 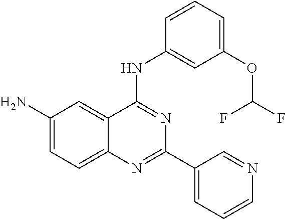 [SMILES: Nc1ccc2nc(-c3cccnc3)nc(Nc3cccc(OC(F)F)c3)c2c1] |
| 57 | 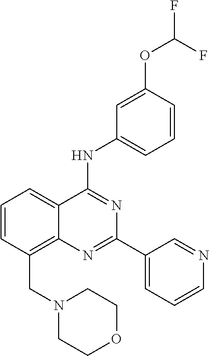 [SMILES: FC(F)Oc1cccc(Nc2nc(-c3cccnc3)nc3c(CN4CCOCC4)cccc23)c1] |
| 58 | 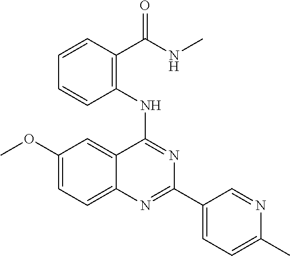 |
| 59 | 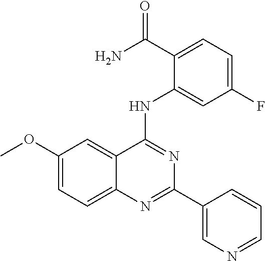 [SMILES: COc1ccc2nc(-c3cccnc3)nc(Nc3cc(F)ccc3C(N)=O)c2c1] |
| 60 | 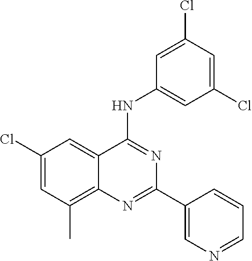 [SMILES: Cc1cc(Cl)cc2c(Nc3cc(Cl)cc(Cl)c3)nc(-c3cccnc3)nc12] |
| 61 | 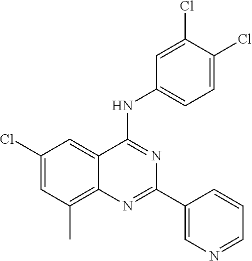 [SMILES: Cc1cc(Cl)cc2c(Nc3ccc(Cl)c(Cl)c3)nc(-c3cccnc3)nc12] |
| 62 | 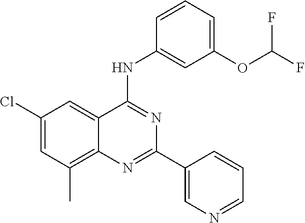 [SMILES: Cc1cc(Cl)cc2c(Nc3cccc(OC(F)F)c3)nc(-c3cccnc3)nc12] |
| 63 | 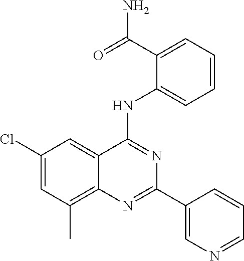 [SMILES: Cc1cc(Cl)cc2c(Nc3ccccc3C(N)=O)nc(-c3cccnc3)nc12] |
| 64 | 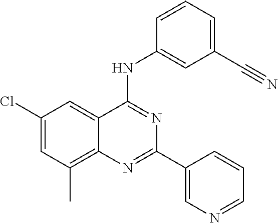 [SMILES: Cc1cc(Cl)cc2c(Nc3cccc(C#N)c3)nc(-c3cccnc3)nc12] |
| 65 | 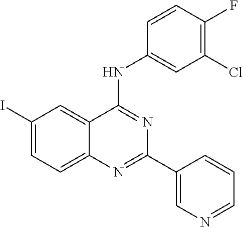 [SMILES: Fc1ccc(Nc2nc(-c3cccnc3)nc3ccc(I)cc23)cc1Cl] |
| 66 | 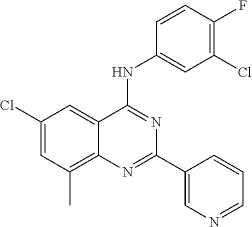 [SMILES: Cc1cc(Cl)cc2c(Nc3ccc(F)c(Cl)c3)nc(-c3cccnc3)nc12] |
| 67 | 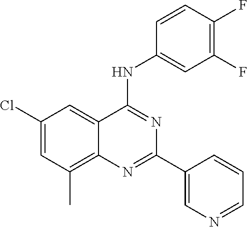 [SMILES: Cc1cc(Cl)cc2c(Nc3ccc(F)c(F)c3)nc(-c3cccnc3)nc12] |
| 68 | 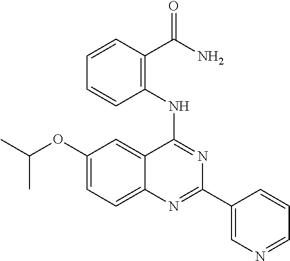 [SMILES: CC(C)Oc1ccc2nc(-c3cccnc3)nc(Nc3ccccc3C(N)=O)c2c1] |
| 69 | 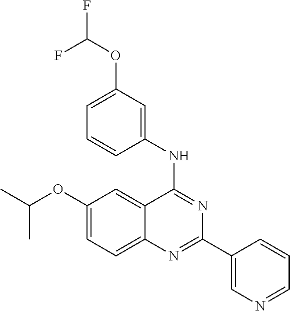 [SMILES: CC(C)Oc1ccc2nc(-c3cccnc3)nc(Nc3cccc(OC(F)F)c3)c2c1] |
| 70 | 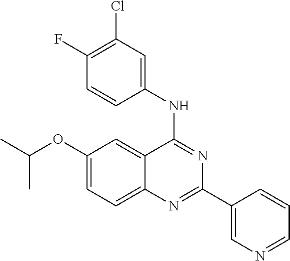 [SMILES: CC(C)Oc1ccc2nc(-c3cccnc3)nc(Nc3ccc(F)c(Cl)c3)c2c1] |
| 71 | 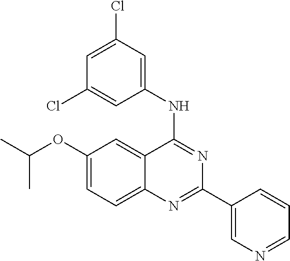 [SMILES: CC(C)Oc1ccc2nc(-c3cccnc3)nc(Nc3cc(Cl)cc(Cl)c3)c2c1] |
| 72 | 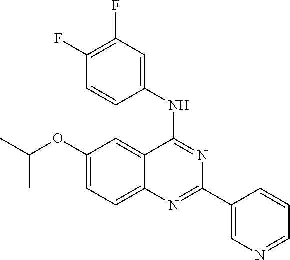 [SMILES: CC(C)Oc1ccc2nc(-c3cccnc3)nc(Nc3ccc(F)c(F)c3)c2c1] |
| 73 | 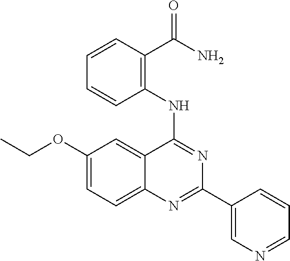 [SMILES: CCOc1ccc2nc(-c3cccnc3)nc(Nc3ccccc3C(N)=O)c2c1] |
| 74 | 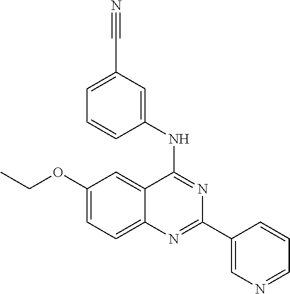 [SMILES: CCOc1ccc2nc(-c3cccnc3)nc(Nc3cccc(C#N)c3)c2c1] |
| 75 | 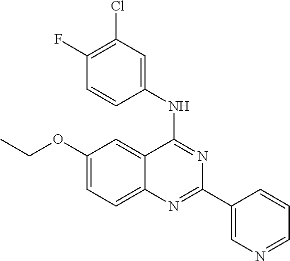 [SMILES: CCOc1ccc2nc(-c3cccnc3)nc(Nc3ccc(F)c(Cl)c3)c2c1] |
| 76 | 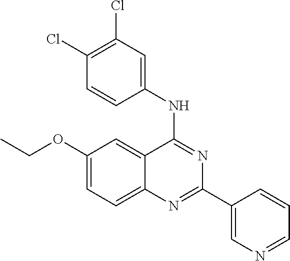 [SMILES: CCOc1ccc2nc(-c3cccnc3)nc(Nc3ccc(Cl)c(Cl)c3)c2c1] |
| 77 | 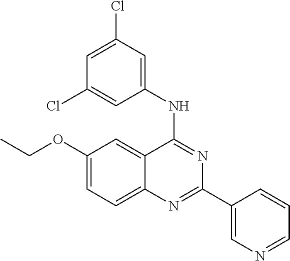 [SMILES: CCOc1ccc2nc(-c3cccnc3)nc(Nc3cc(Cl)cc(Cl)c3)c2c1] |
| 78 | 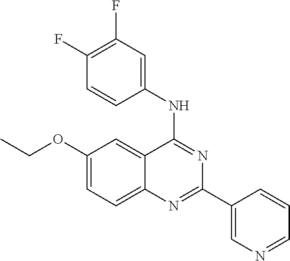 [SMILES: CCOc1ccc2nc(-c3cccnc3)nc(Nc3ccc(F)c(F)c3)c2c1] |
| 79 | 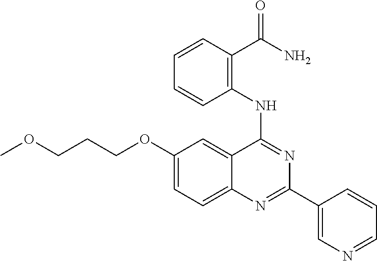 [SMILES: COCCCOc1ccc2nc(-c3cccnc3)nc(Nc3ccccc3C(N)=O)c2c1] |
| 80 | 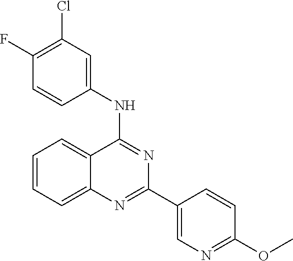 [SMILES: COc1ccc(-c2nc(Nc3ccc(F)c(Cl)c3)c3ccccc3n2)cn1] |
| 81 | 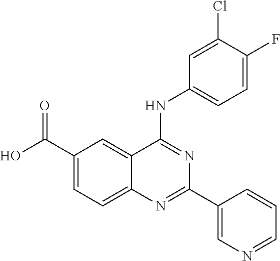 [SMILES: O=C(O)c1ccc2nc(-c3cccnc3)nc(Nc3ccc(F)c(Cl)c3)c2c1] |
| 82 | 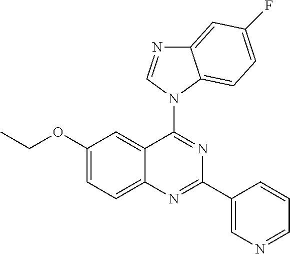 [SMILES: CCOc1ccc2nc(-c3cccnc3)nc(-n3cnc4cc(F)ccc43)c2c1] |
| 83 | 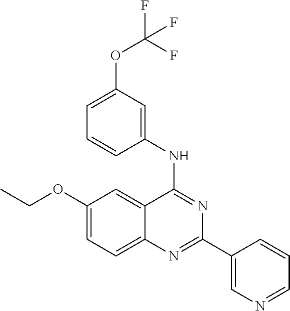 [SMILES: CCOc1ccc2nc(-c3cccnc3)nc(Nc3cccc(OC(F)(F)F)c3)c2c1] |
| 84 | 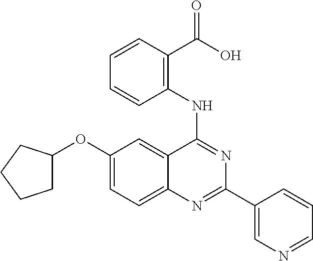 [SMILES: O=C(O)c1ccccc1Nc1nc(-c2cccnc2)nc2ccc(OC3CCCC3)cc12] |
| 85 | 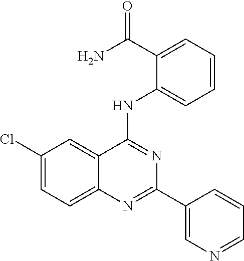 [SMILES: NC(=O)c1ccccc1Nc1nc(-c2cccnc2)nc2ccc(Cl)cc12] |
| 86 | 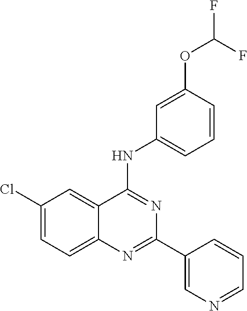 [SMILES: FC(F)Oc1cccc(Nc2nc(-c3cccnc3)nc3ccc(Cl)cc23)c1] |
| 87 | 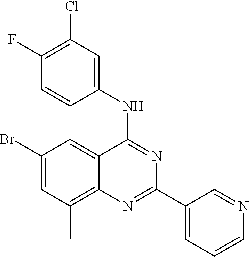 [SMILES: Cc1cc(Br)cc2c(Nc3ccc(F)c(Cl)c3)nc(-c3cccnc3)nc12] |
| 88 | 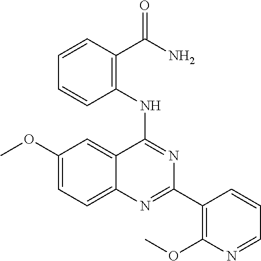 [SMILES: COc1ccc2nc(-c3cccnc3OC)nc(Nc3ccccc3C(N)=O)c2c1] |
| 89 | 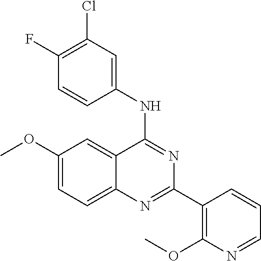 [SMILES: COc1ccc2nc(-c3cccnc3OC)nc(Nc3ccc(F)c(Cl)c3)c2c1] |
| 90 | 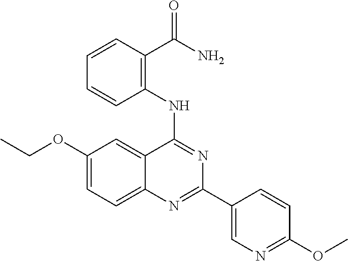 [SMILES: CCOc1ccc2nc(-c3ccc(OC)nc3)nc(Nc3ccccc3C(N)=O)c2c1] |
| 91 | 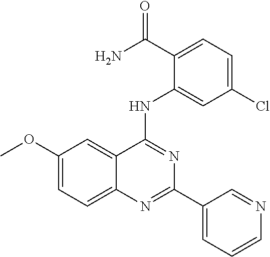 [SMILES: COc1ccc2nc(-c3cccnc3)nc(Nc3cc(Cl)ccc3C(N)=O)c2c1] |
| 92 | 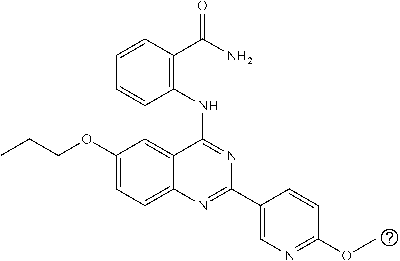 [SMILES: CCCOc1ccc2nc(-c3ccc(OC)nc3)nc(Nc3ccccc3C(N)=O)c2c1] |
| 93 | 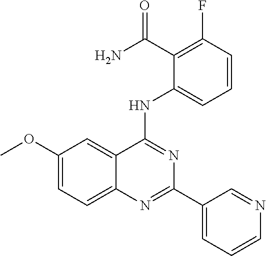 [SMILES: COc1ccc2nc(-c3cccnc3)nc(Nc3cccc(F)c3C(N)=O)c2c1] |
| 94 | 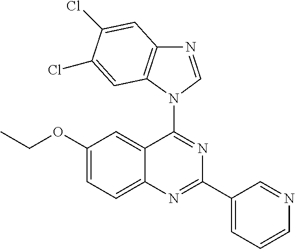 [SMILES: CCOc1ccc2nc(-c3cccnc3)nc(-n3cnc4cc(Cl)c(Cl)cc43)c2c1] |
| 95 | 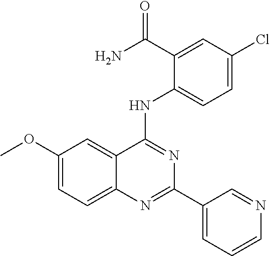 [SMILES: COc1ccc2nc(-c3cccnc3)nc(Nc3ccc(Cl)cc3C(N)=O)c2c1] |
| 96 | 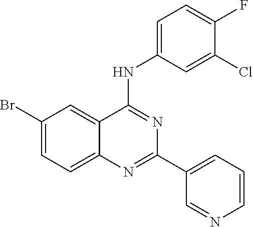 [SMILES: Fc1ccc(Nc2nc(-c3cccnc3)nc3ccc(Br)cc23)cc1Cl] |
| 97 | 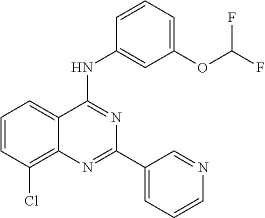 [SMILES: FC(F)Oc1cccc(Nc2nc(-c3cccnc3)nc3c(Cl)cccc23)c1] |
| 98 | 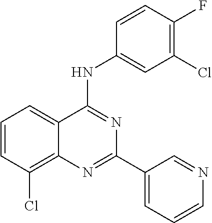 [SMILES: Fc1ccc(Nc2nc(-c3cccnc3)nc3c(Cl)cccc23)cc1Cl] |
| 99 | 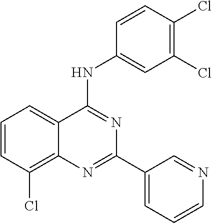 [SMILES: Clc1ccc(Nc2nc(-c3cccnc3)nc3c(Cl)cccc23)cc1Cl] |
| 100 | 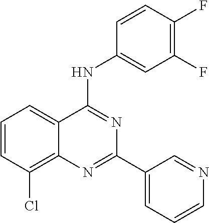 [SMILES: Fc1ccc(Nc2nc(-c3cccnc3)nc3c(Cl)cccc23)cc1F] |
| 101 | 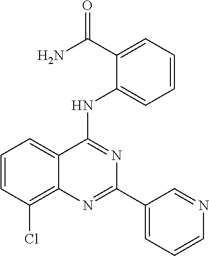 [SMILES: NC(=O)c1ccccc1Nc1nc(-c2cccnc2)nc2c(Cl)cccc12] |
| 102 | 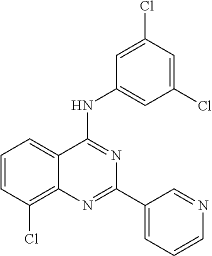 [SMILES: Clc1cc(Cl)cc(Nc2nc(-c3cccnc3)nc3c(Cl)cccc23)c1] |
| 103 | 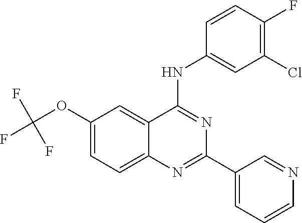 [SMILES: Fc1ccc(Nc2nc(-c3cccnc3)nc3ccc(OC(F)(F)F)cc23)cc1Cl] |
| 104 | 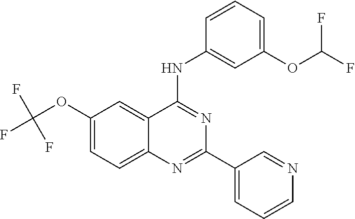 [SMILES: FC(F)Oc1cccc(Nc2nc(-c3cccnc3)nc3ccc(OC(F)(F)F)cc23)c1] |
| 105 | 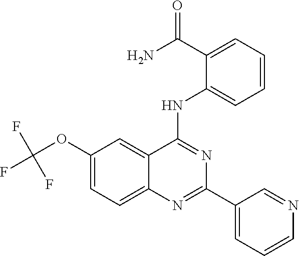 [SMILES: NC(=O)c1ccccc1Nc1nc(-c2cccnc2)nc2ccc(OC(F)(F)F)cc12] |
| 106 | 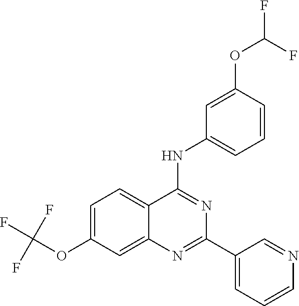 [SMILES: FC(F)Oc1cccc(Nc2nc(-c3cccnc3)nc3cc(OC(F)(F)F)ccc23)c1] |
| 107 | 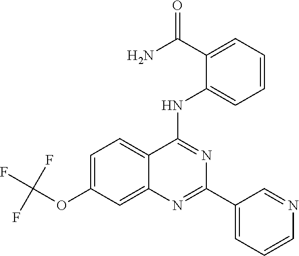 [SMILES: NC(=O)c1ccccc1Nc1nc(-c2cccnc2)nc2cc(OC(F)(F)F)ccc12] |
| 108 | 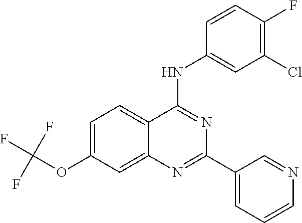 [SMILES: Fc1ccc(Nc2nc(-c3cccnc3)nc3cc(OC(F)(F)F)ccc23)cc1Cl] |
| 109 | 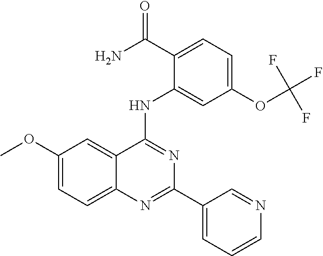 [SMILES: COc1ccc2nc(-c3cccnc3)nc(Nc3cc(OC(F)(F)F)ccc3C(N)=O)c2c1] |
| 110 | 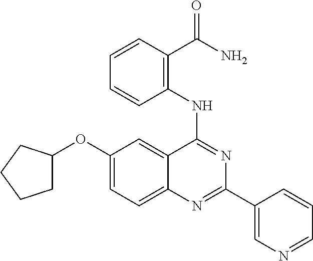 [SMILES: NC(=O)c1ccccc1Nc1nc(-c2cccnc2)nc2ccc(OC3CCCC3)cc12] |
| 111 | 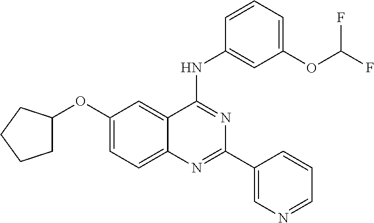 [SMILES: FC(F)Oc1cccc(Nc2nc(-c3cccnc3)nc3ccc(OC4CCCC4)cc23)c1] |
| 112 | 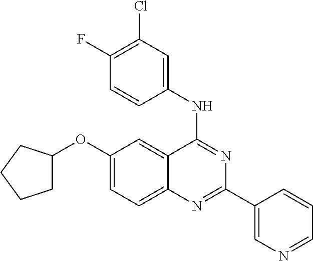 [SMILES: Fc1ccc(Nc2nc(-c3cccnc3)nc3ccc(OC4CCCC4)cc23)cc1Cl] |
| 113 | 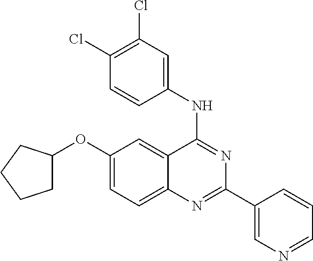 [SMILES: Clc1ccc(Nc2nc(-c3cccnc3)nc3ccc(OC4CCCC4)cc23)cc1Cl] |
| 114 | 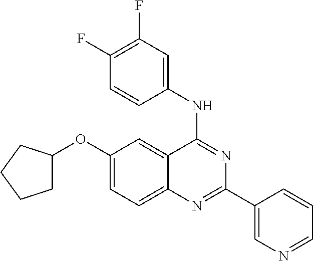 [SMILES: Fc1ccc(Nc2nc(-c3cccnc3)nc3ccc(OC4CCCC4)cc23)cc1F] |
| 115 | 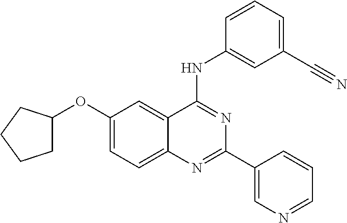 [SMILES: N#Cc1cccc(Nc2nc(-c3cccnc3)nc3ccc(OC4CCCC4)cc23)c1] |
| 116 | 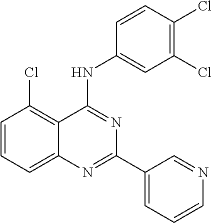 [SMILES: Clc1ccc(Nc2nc(-c3cccnc3)nc3cccc(Cl)c23)cc1Cl] |
| 117 | 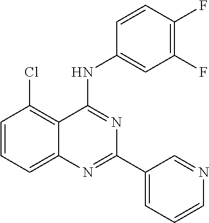 [SMILES: Fc1ccc(Nc2nc(-c3cccnc3)nc3cccc(Cl)c23)cc1F] |
| 118 | 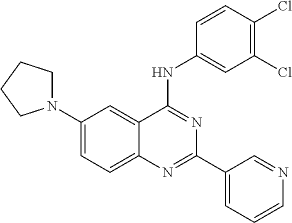 [SMILES: Clc1ccc(Nc2nc(-c3cccnc3)nc3ccc(N4CCCC4)cc23)cc1Cl] |
| 119 | 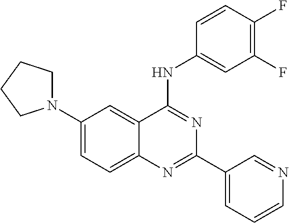 [SMILES: Fc1ccc(Nc2nc(-c3cccnc3)nc3ccc(N4CCCC4)cc23)cc1F] |
| 120 | 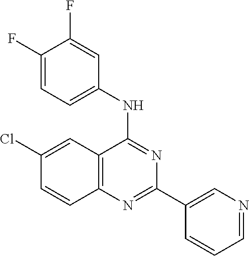 [SMILES: Fc1ccc(Nc2nc(-c3cccnc3)nc3ccc(Cl)cc23)cc1F] |
| 121 | 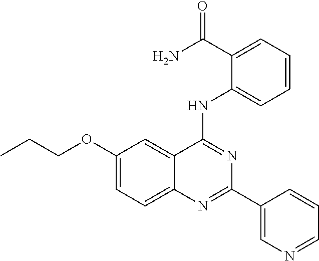 [SMILES: CCCOc1ccc2nc(-c3cccnc3)nc(Nc3ccccc3C(N)=O)c2c1] |
| 122 | 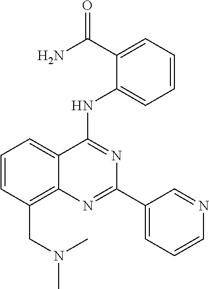 [SMILES: CN(C)Cc1cccc2c(Nc3ccccc3C(N)=O)nc(-c3cccnc3)nc12] |
| 123 | 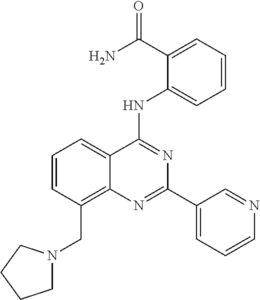 [SMILES: NC(=O)c1ccccc1Nc1nc(-c2cccnc2)nc2c(CN3CCCC3)cccc12] |
| 124 | 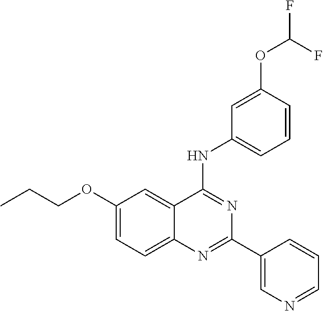 [SMILES: CCCOc1ccc2nc(-c3cccnc3)nc(Nc3cccc(OC(F)F)c3)c2c1] |
| 125 | 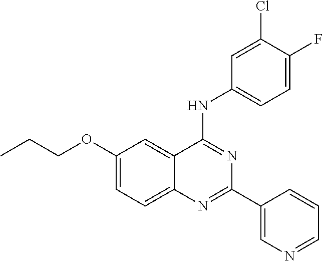 [SMILES: CCCOc1ccc2nc(-c3cccnc3)nc(Nc3ccc(F)c(Cl)c3)c2c1] |
| 126 | 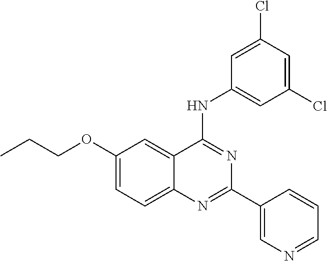 [SMILES: CCCOc1ccc2nc(-c3cccnc3)nc(Nc3cc(Cl)cc(Cl)c3)c2c1] |
| 127 | 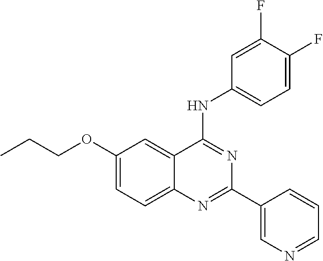 [SMILES: CCCOc1ccc2nc(-c3cccnc3)nc(Nc3ccc(F)c(F)c3)c2c1] |
| 128 | 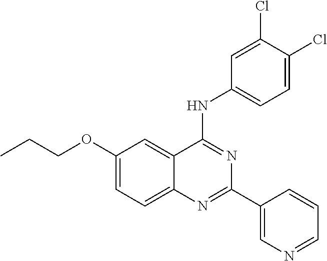 [SMILES: CCCOc1ccc2nc(-c3cccnc3)nc(Nc3ccc(Cl)c(Cl)c3)c2c1] |
| 129 | 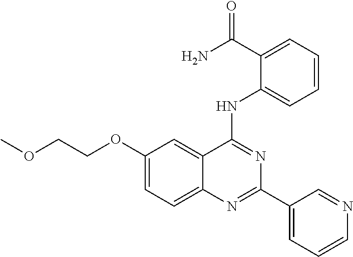 [SMILES: COCCOc1ccc2nc(-c3cccnc3)nc(Nc3ccccc3C(N)=O)c2c1] |
| 130 | 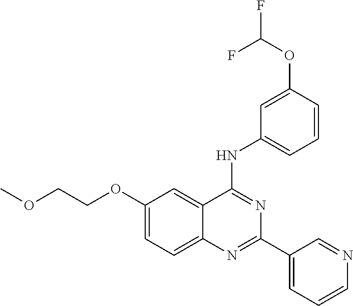 [SMILES: COCCOc1ccc2nc(-c3cccnc3)nc(Nc3cccc(OC(F)F)c3)c2c1] |
| 131 | 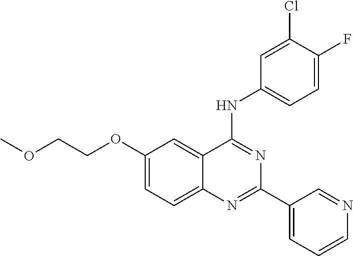 [SMILES: COCCOc1ccc2nc(-c3cccnc3)nc(Nc3ccc(F)c(Cl)c3)c2c1] |
| 132 | 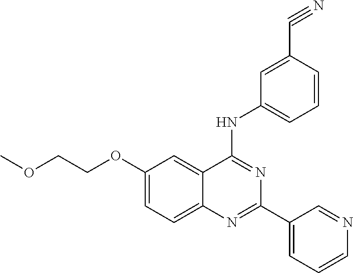 [SMILES: COCCOc1ccc2nc(-c3cccnc3)nc(Nc3cccc(C#N)c3)c2c1] |
| 133 | 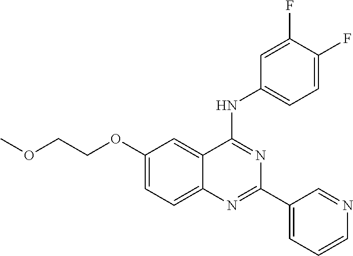 [SMILES: COCCOc1ccc2nc(-c3cccnc3)nc(Nc3ccc(F)c(F)c3)c2c1] |
| 134 | 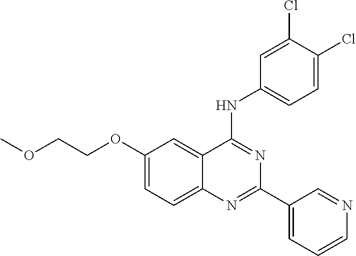 [SMILES: COCCOc1ccc2nc(-c3cccnc3)nc(Nc3ccc(Cl)c(Cl)c3)c2c1] |
| 135 | 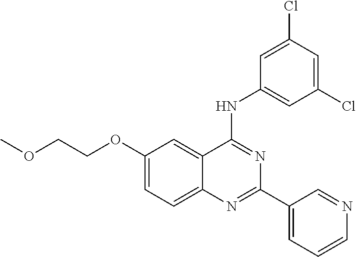 [SMILES: COCCOc1ccc2nc(-c3cccnc3)nc(Nc3cc(Cl)cc(Cl)c3)c2c1] |
| 136 | 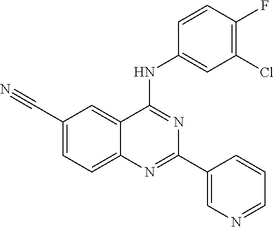 [SMILES: N#Cc1ccc2nc(-c3cccnc3)nc(Nc3ccc(F)c(Cl)c3)c2c1] |
| 137 | 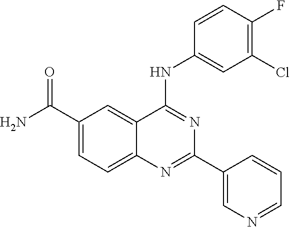 [SMILES: NC(=O)c1ccc2nc(-c3cccnc3)nc(Nc3ccc(F)c(Cl)c3)c2c1] |
| 138 | 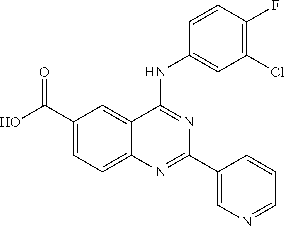 [SMILES: O=C(O)c1ccc2nc(-c3cccnc3)nc(Nc3ccc(F)c(Cl)c3)c2c1] |
| 139 | 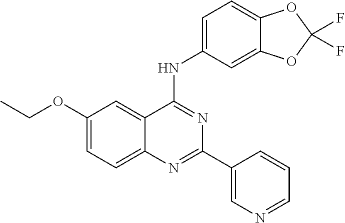 [SMILES: CCOc1ccc2nc(-c3cccnc3)nc(Nc3ccc4c(c3)OC(F)(F)O4)c2c1] |
| 140 | 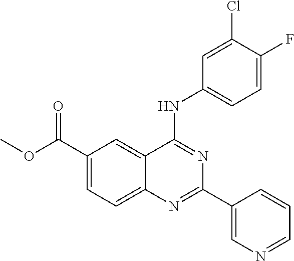 [SMILES: COC(=O)c1ccc2nc(-c3cccnc3)nc(Nc3ccc(F)c(Cl)c3)c2c1] |
| 141 | 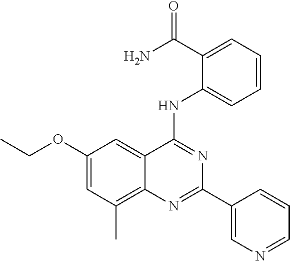 [SMILES: CCOc1cc(C)c2nc(-c3cccnc3)nc(Nc3ccccc3C(N)=O)c2c1] |
| 142 | 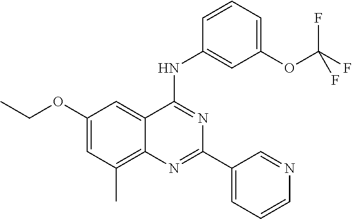 [SMILES: CCOc1cc(C)c2nc(-c3cccnc3)nc(Nc3cccc(OC(F)(F)F)c3)c2c1] |
| 143 | 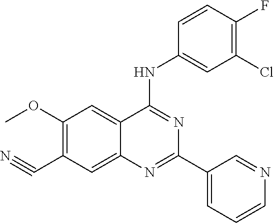 [SMILES: COc1cc2c(Nc3ccc(F)c(Cl)c3)nc(-c3cccnc3)nc2cc1C#N] |
| 144 | 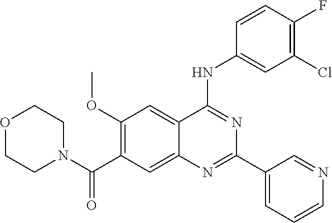 [SMILES: COc1cc2c(Nc3ccc(F)c(Cl)c3)nc(-c3cccnc3)nc2cc1C(=O)N1CCOCC1] |
| 145 | 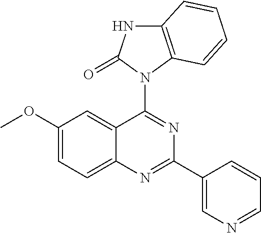 [SMILES: COc1ccc2nc(-c3cccnc3)nc(-n3c(=O)[nH]c4ccccc43)c2c1] |
| 146 | 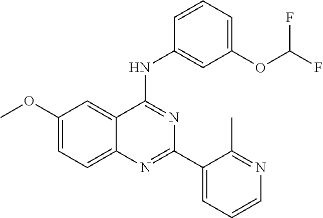 [SMILES: COc1ccc2nc(-c3cccnc3C)nc(Nc3cccc(OC(F)F)c3)c2c1] |
| 147 | 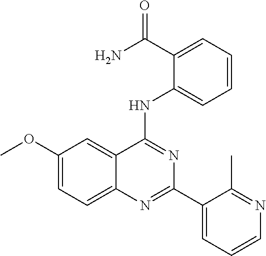 [SMILES: COc1ccc2nc(-c3cccnc3C)nc(Nc3ccccc3C(N)=O)c2c1] |
| 148 | 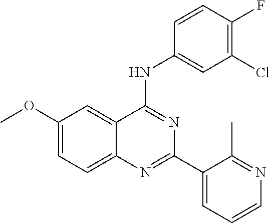 [SMILES: COc1ccc2nc(-c3cccnc3C)nc(Nc3ccc(F)c(Cl)c3)c2c1] |
| 149 | 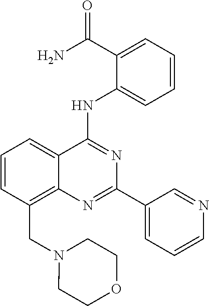 [SMILES: NC(=O)c1ccccc1Nc1nc(-c2cccnc2)nc2c(CN3CCOCC3)cccc12] |
| 150 | 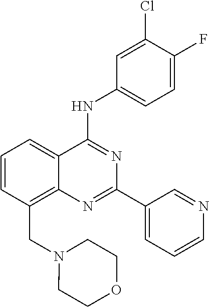 [SMILES: Fc1ccc(Nc2nc(-c3cccnc3)nc3c(CN4CCOCC4)cccc23)cc1Cl] |
| 151 | 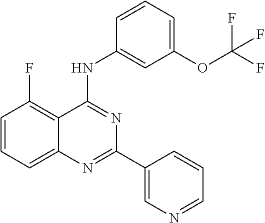 [SMILES: Fc1cccc2nc(-c3cccnc3)nc(Nc3cccc(OC(F)(F)F)c3)c12] |
| 152 | 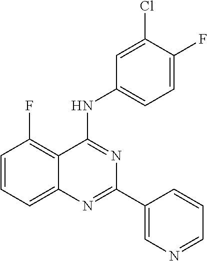 [SMILES: Fc1ccc(Nc2nc(-c3cccnc3)nc3cccc(F)c23)cc1Cl] |
| 153 | 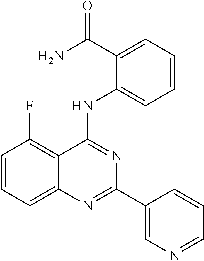 [SMILES: NC(=O)c1ccccc1Nc1nc(-c2cccnc2)nc2cccc(F)c12] |
| 154 | 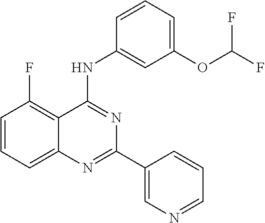 [SMILES: Fc1cccc2nc(-c3cccnc3)nc(Nc3cccc(OC(F)F)c3)c12] |
| 155 | 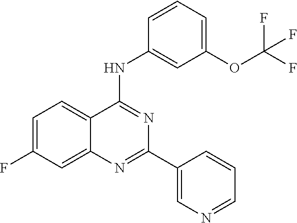 [SMILES: Fc1ccc2c(Nc3cccc(OC(F)(F)F)c3)nc(-c3cccnc3)nc2c1] |
| 156 | 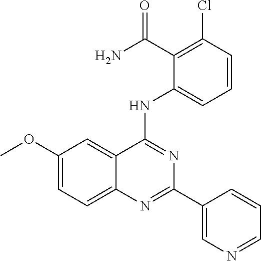 [SMILES: COc1ccc2nc(-c3cccnc3)nc(Nc3cccc(Cl)c3C(N)=O)c2c1] |
| 157 | 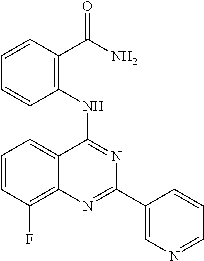 [SMILES: NC(=O)c1ccccc1Nc1nc(-c2cccnc2)nc2c(F)cccc12] |
| 158 | 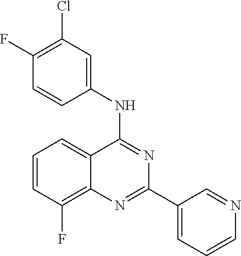 [SMILES: Fc1ccc(Nc2nc(-c3cccnc3)nc3c(F)cccc23)cc1Cl] |
| 159 | 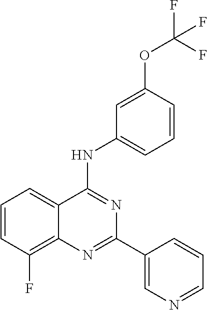 [SMILES: Fc1cccc2c(Nc3cccc(OC(F)(F)F)c3)nc(-c3cccnc3)nc12] |
| 160 | 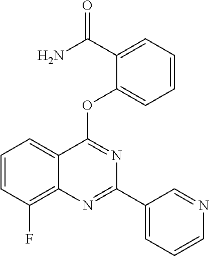 [SMILES: NC(=O)c1ccccc1Oc1nc(-c2cccnc2)nc2c(F)cccc12] |
| 161 | 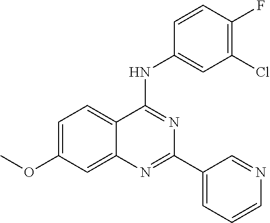 [SMILES: COc1ccc2c(Nc3ccc(F)c(Cl)c3)nc(-c3cccnc3)nc2c1] |
| 162 | 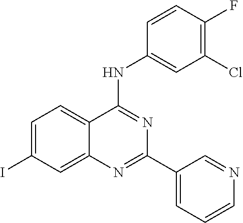 [SMILES: Fc1ccc(Nc2nc(-c3cccnc3)nc3cc(I)ccc23)cc1Cl] |
| 163 | 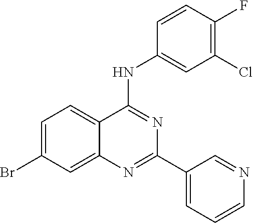 [SMILES: Fc1ccc(Nc2nc(-c3cccnc3)nc3cc(Br)ccc23)cc1Cl] |
| 164 | 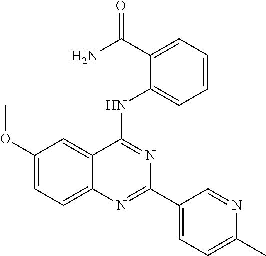 [SMILES: COc1ccc2nc(-c3ccc(C)nc3)nc(Nc3ccccc3C(N)=O)c2c1] |
| 165 | 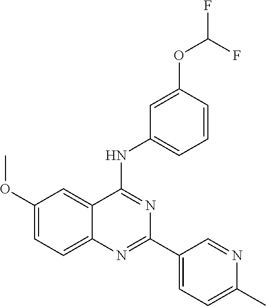 [SMILES: COc1ccc2nc(-c3ccc(C)nc3)nc(Nc3cccc(OC(F)F)c3)c2c1] |
| 166 | 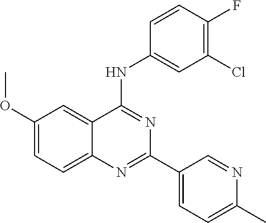 [SMILES: COc1ccc2nc(-c3ccc(C)nc3)nc(Nc3ccc(F)c(Cl)c3)c2c1] |
| 167 | 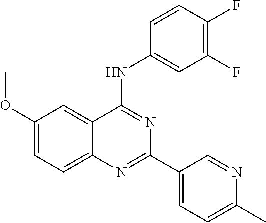 [SMILES: COc1ccc2nc(-c3ccc(C)nc3)nc(Nc3ccc(F)c(F)c3)c2c1] |
| 168 | 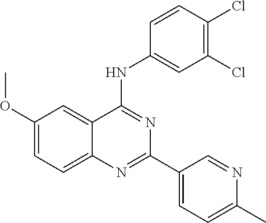 [SMILES: COc1ccc2nc(-c3ccc(C)nc3)nc(Nc3ccc(Cl)c(Cl)c3)c2c1] |
| 169 | 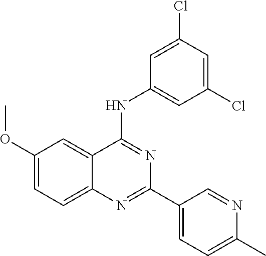 [SMILES: COc1ccc2nc(-c3ccc(C)nc3)nc(Nc3cc(Cl)cc(Cl)c3)c2c1] |
| 170 | 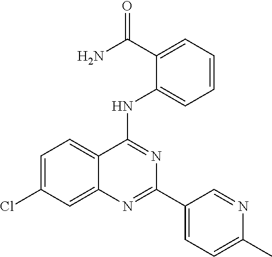 [SMILES: Cc1ccc(-c2nc(Nc3ccccc3C(N)=O)c3ccc(Cl)cc3n2)cn1] |
| 171 | 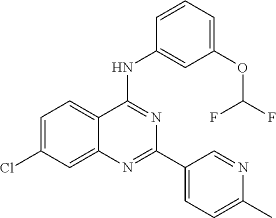 [SMILES: Cc1ccc(-c2nc(Nc3cccc(OC(F)F)c3)c3ccc(Cl)cc3n2)cn1] |
| 172 | 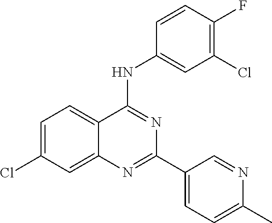 [SMILES: Cc1ccc(-c2nc(Nc3ccc(F)c(Cl)c3)c3ccc(Cl)cc3n2)cn1] |
| 173 | 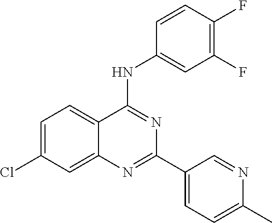 [SMILES: Cc1ccc(-c2nc(Nc3ccc(F)c(F)c3)c3ccc(Cl)cc3n2)cn1] |
| 174 | 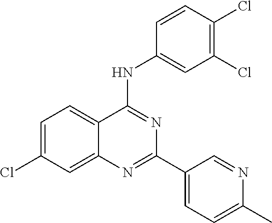 [SMILES: Cc1ccc(-c2nc(Nc3ccc(Cl)c(Cl)c3)c3ccc(Cl)cc3n2)cn1] |
| 175 | 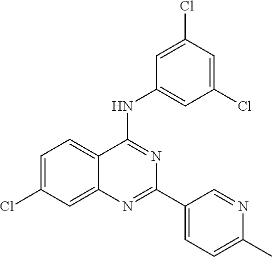 [SMILES: Cc1ccc(-c2nc(Nc3cc(Cl)cc(Cl)c3)c3ccc(Cl)cc3n2)cn1] |
| 176 | 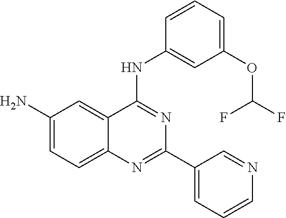 [SMILES: Nc1ccc2nc(-c3cccnc3)nc(Nc3cccc(OC(F)F)c3)c2c1] |
| 177 | 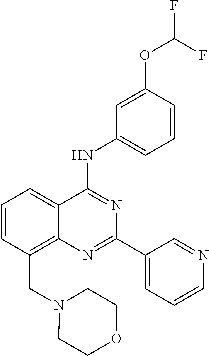 [SMILES: FC(F)Oc1cccc(Nc2nc(-c3cccnc3)nc3c(CN4CCOCC4)cccc23)c1] |
| 178 | 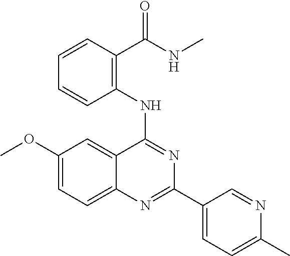 |
| 179 | 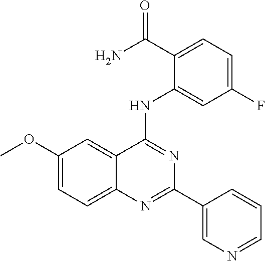 [SMILES: COc1ccc2nc(-c3cccnc3)nc(Nc3cc(F)ccc3C(N)=O)c2c1] |
| 180 | 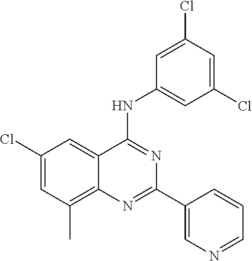 [SMILES: Cc1cc(Cl)cc2c(Nc3cc(Cl)cc(Cl)c3)nc(-c3cccnc3)nc12] |
| 181 | 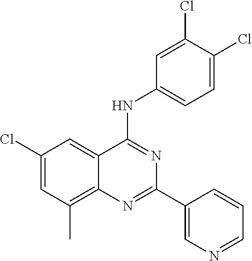 [SMILES: Cc1cc(Cl)cc2c(Nc3ccc(Cl)c(Cl)c3)nc(-c3cccnc3)nc12] |
| 182 | 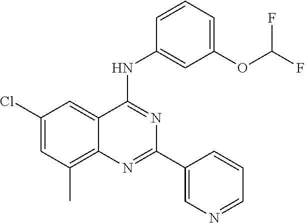 [SMILES: Cc1cc(Cl)cc2c(Nc3cccc(OC(F)F)c3)nc(-c3cccnc3)nc12] |
| 183 | 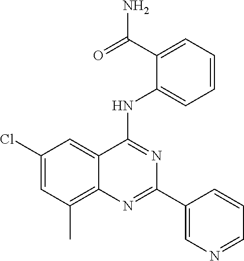 [SMILES: Cc1cc(Cl)cc2c(Nc3ccccc3C(N)=O)nc(-c3cccnc3)nc12] |
| 184 | 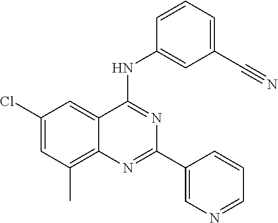 [SMILES: Cc1cc(Cl)cc2c(Nc3cccc(C#N)c3)nc(-c3cccnc3)nc12] |
| 185 | 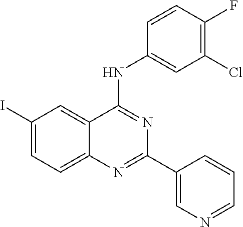 [SMILES: Fc1ccc(Nc2nc(-c3cccnc3)nc3ccc(I)cc23)cc1Cl] |
| 186 | 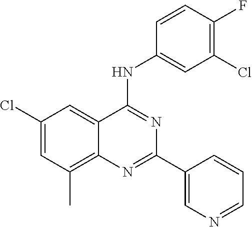 [SMILES: Cc1cc(Cl)cc2c(Nc3ccc(F)c(Cl)c3)nc(-c3cccnc3)nc12] |
| 187 | 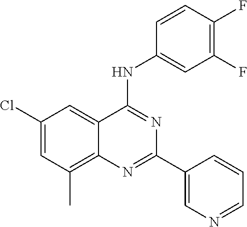 [SMILES: Cc1cc(Cl)cc2c(Nc3ccc(F)c(F)c3)nc(-c3cccnc3)nc12] |
| 188 | 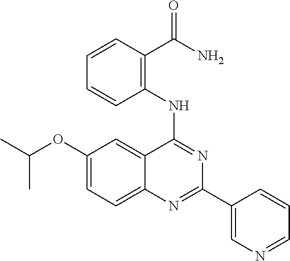 [SMILES: CC(C)Oc1ccc2nc(-c3cccnc3)nc(Nc3ccccc3C(N)=O)c2c1] |
| 189 | 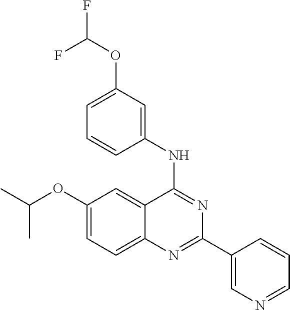 [SMILES: CC(C)Oc1ccc2nc(-c3cccnc3)nc(Nc3cccc(OC(F)F)c3)c2c1] |
| 190 | 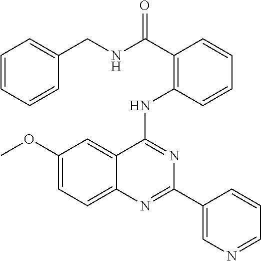 [SMILES: COc1ccc2nc(-c3cccnc3)nc(Nc3ccccc3C(=O)NCc3ccccc3)c2c1] |
| 191 | 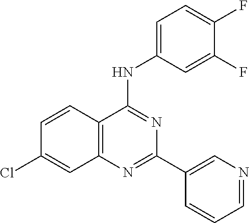 [SMILES: Fc1ccc(Nc2nc(-c3cccnc3)nc3cc(Cl)ccc23)cc1F] |
| 192 | 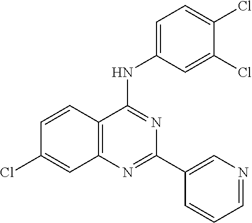 [SMILES: Clc1ccc2c(Nc3ccc(Cl)c(Cl)c3)nc(-c3cccnc3)nc2c1] |
| 193 | 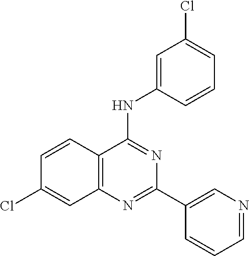 [SMILES: Clc1cccc(Nc2nc(-c3cccnc3)nc3cc(Cl)ccc23)c1] |
| 194 | 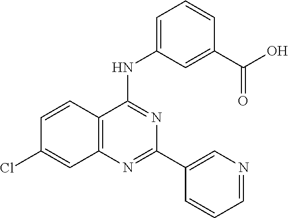 [SMILES: O=C(O)c1cccc(Nc2nc(-c3cccnc3)nc3cc(Cl)ccc23)c1] |
| 195 | 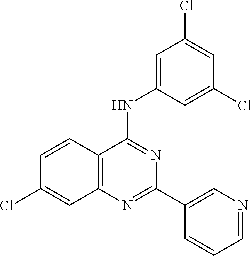 [SMILES: Clc1cc(Cl)cc(Nc2nc(-c3cccnc3)nc3cc(Cl)ccc23)c1] |
| 196 | 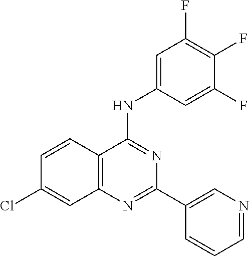 [SMILES: Fc1cc(Nc2nc(-c3cccnc3)nc3cc(Cl)ccc23)cc(F)c1F] |
| 197 | 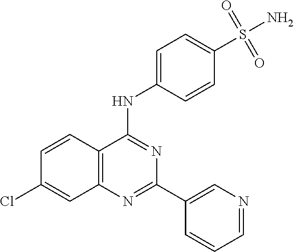 [SMILES: NS(=O)(=O)c1ccc(Nc2nc(-c3cccnc3)nc3cc(Cl)ccc23)cc1] |
| 198 | 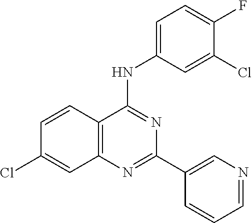 [SMILES: Fc1ccc(Nc2nc(-c3cccnc3)nc3cc(Cl)ccc23)cc1Cl] |
| 199 | 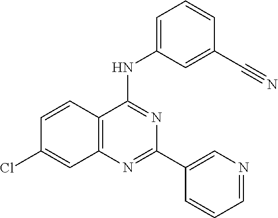 [SMILES: N#Cc1cccc(Nc2nc(-c3cccnc3)nc3cc(Cl)ccc23)c1] |
| 200 | 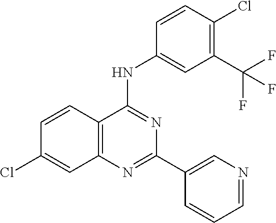 [SMILES: FC(F)(F)c1cc(Nc2nc(-c3cccnc3)nc3cc(Cl)ccc23)ccc1Cl] |
| 201 | 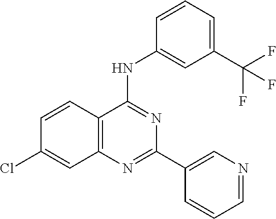 [SMILES: FC(F)(F)c1cccc(Nc2nc(-c3cccnc3)nc3cc(Cl)ccc23)c1] |
| 202 | 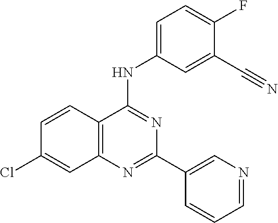 [SMILES: N#Cc1cc(Nc2nc(-c3cccnc3)nc3cc(Cl)ccc23)ccc1F] |
| 203 | 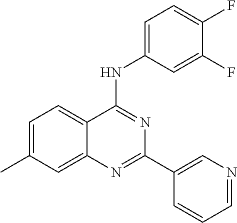 [SMILES: Cc1ccc2c(Nc3ccc(F)c(F)c3)nc(-c3cccnc3)nc2c1] |
| 204 | 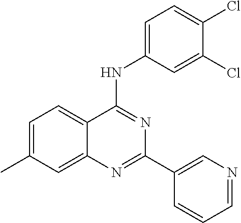 [SMILES: Cc1ccc2c(Nc3ccc(Cl)c(Cl)c3)nc(-c3cccnc3)nc2c1] |
| 205 | 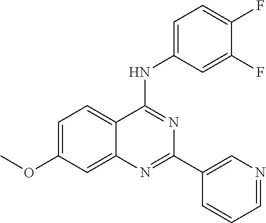 [SMILES: COc1ccc2c(Nc3ccc(F)c(F)c3)nc(-c3cccnc3)nc2c1] |
| 206 | 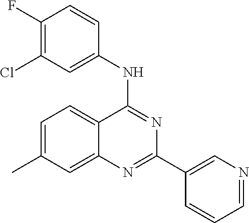 [SMILES: Cc1ccc2c(Nc3ccc(F)c(Cl)c3)nc(-c3cccnc3)nc2c1] |
| 207 | 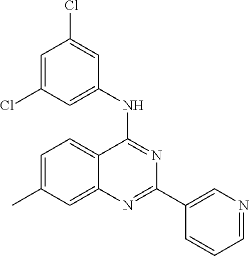 [SMILES: Cc1ccc2c(Nc3cc(Cl)cc(Cl)c3)nc(-c3cccnc3)nc2c1] |
| 208 | 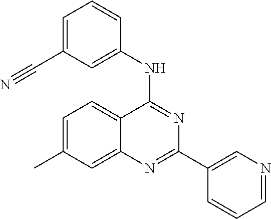 [SMILES: Cc1ccc2c(Nc3cccc(C#N)c3)nc(-c3cccnc3)nc2c1] |
| 209 | 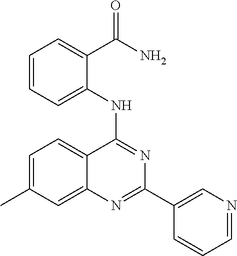 [SMILES: Cc1ccc2c(Nc3ccccc3C(N)=O)nc(-c3cccnc3)nc2c1] |
| 210 | 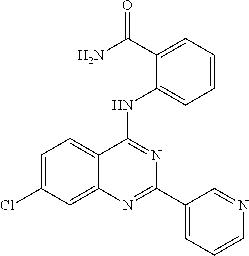 [SMILES: NC(=O)c1ccccc1Nc1nc(-c2cccnc2)nc2cc(Cl)ccc12] |
| 211 | 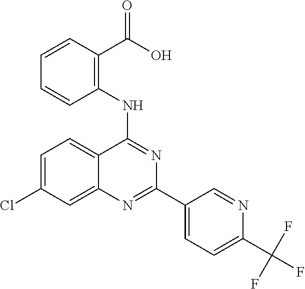 [SMILES: O=C(O)c1ccccc1Nc1nc(-c2ccc(C(F)(F)F)nc2)nc2cc(Cl)ccc12] |
| 212 | 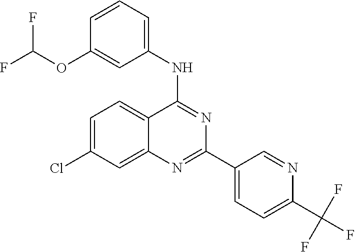 [SMILES: FC(F)Oc1cccc(Nc2nc(-c3ccc(C(F)(F)F)nc3)nc3cc(Cl)ccc23)c1] |
| 213 | 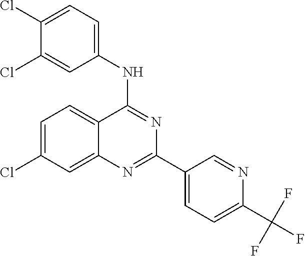 [SMILES: FC(F)(F)c1ccc(-c2nc(Nc3ccc(Cl)c(Cl)c3)c3ccc(Cl)cc3n2)cn1] |
| 214 | 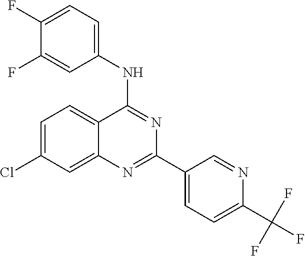 [SMILES: Fc1ccc(Nc2nc(-c3ccc(C(F)(F)F)nc3)nc3cc(Cl)ccc23)cc1F] |
| 215 | 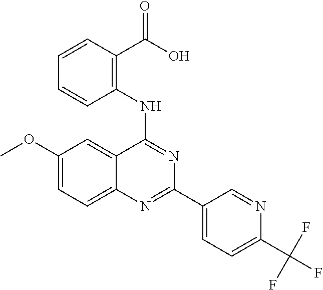 [SMILES: COc1ccc2nc(-c3ccc(C(F)(F)F)nc3)nc(Nc3ccccc3C(=O)O)c2c1] |
| 216 | 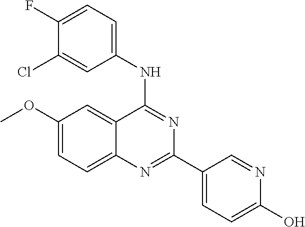 [SMILES: COc1ccc2nc(-c3ccc(O)nc3)nc(Nc3ccc(F)c(Cl)c3)c2c1] |
| 217 | 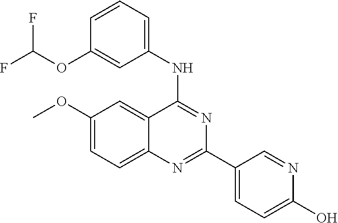 [SMILES: COc1ccc2nc(-c3ccc(O)nc3)nc(Nc3cccc(OC(F)F)c3)c2c1] |
| 218 | 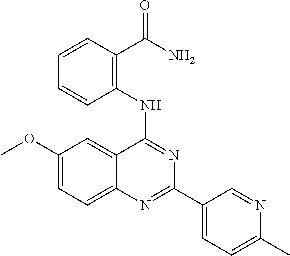 |
| 219 | 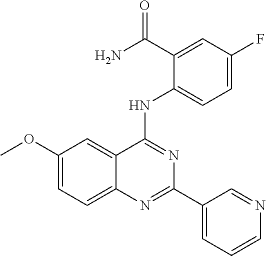 [SMILES: COc1ccc2nc(-c3cccnc3)nc(Nc3ccc(F)cc3C(N)=O)c2c1] |
| 220 | 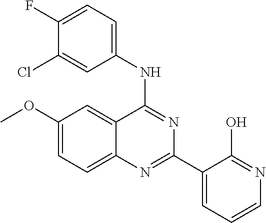 [SMILES: COc1ccc2nc(-c3cccnc3O)nc(Nc3ccc(F)c(Cl)c3)c2c1] |
| 221 | 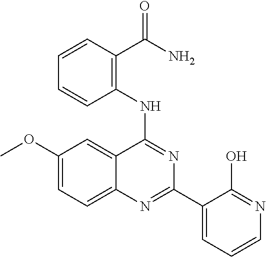 [SMILES: COc1ccc2nc(-c3cccnc3O)nc(Nc3ccccc3C(N)=O)c2c1] |
| 222 | 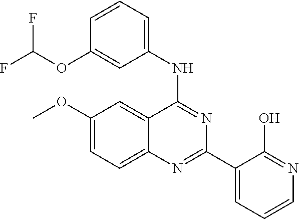 [SMILES: COc1ccc2nc(-c3cccnc3O)nc(Nc3cccc(OC(F)F)c3)c2c1] |
| 223 | 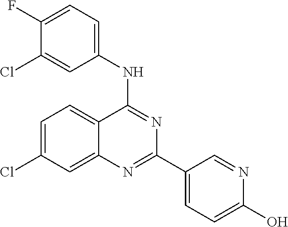 [SMILES: Oc1ccc(-c2nc(Nc3ccc(F)c(Cl)c3)c3ccc(Cl)cc3n2)cn1] |
| 224 | 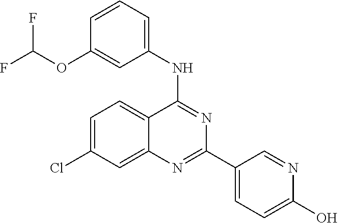 [SMILES: Oc1ccc(-c2nc(Nc3cccc(OC(F)F)c3)c3ccc(Cl)cc3n2)cn1] |
| 225 | 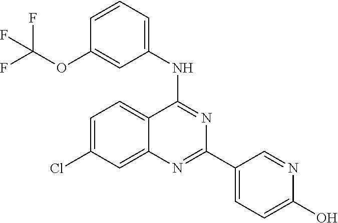 [SMILES: Oc1ccc(-c2nc(Nc3cccc(OC(F)(F)F)c3)c3ccc(Cl)cc3n2)cn1] |
| 226 | 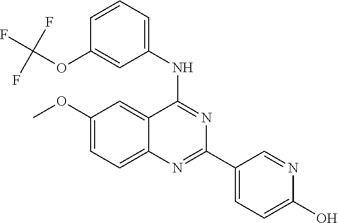 [SMILES: COc1ccc2nc(-c3ccc(O)nc3)nc(Nc3cccc(OC(F)(F)F)c3)c2c1] |
| 227 | 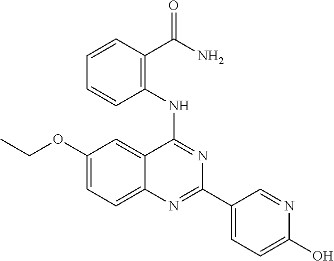 [SMILES: CCOc1ccc2nc(-c3ccc(O)nc3)nc(Nc3ccccc3C(N)=O)c2c1] |
| 228 | 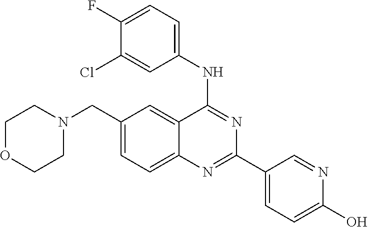 [SMILES: Oc1ccc(-c2nc(Nc3ccc(F)c(Cl)c3)c3cc(CN4CCOCC4)ccc3n2)cn1] |
| 229 | 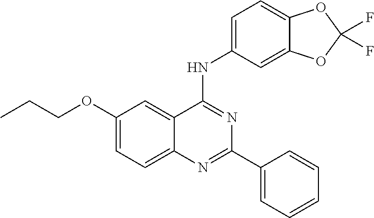 [SMILES: CCCOc1ccc2nc(-c3ccccc3)nc(Nc3ccc4c(c3)OC(F)(F)O4)c2c1] |
| 230 | 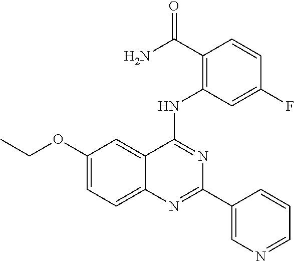 [SMILES: CCOc1ccc2nc(-c3cccnc3)nc(Nc3cc(F)ccc3C(N)=O)c2c1] |
| 231 | 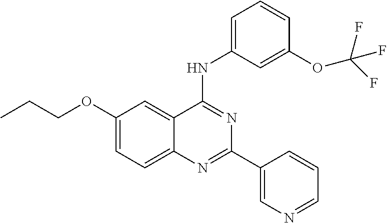 [SMILES: CCCOc1ccc2nc(-c3cccnc3)nc(Nc3cccc(OC(F)(F)F)c3)c2c1] |
| 232 | 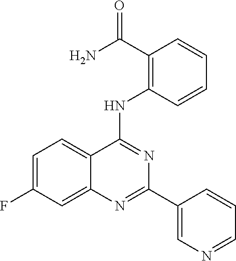 [SMILES: NC(=O)c1ccccc1Nc1nc(-c2cccnc2)nc2cc(F)ccc12] |
| 233 | 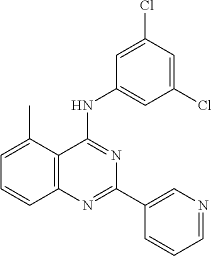 [SMILES: Cc1cccc2nc(-c3cccnc3)nc(Nc3cc(Cl)cc(Cl)c3)c12] |
| 234 | 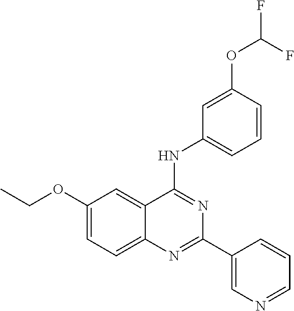 [SMILES: CCOc1ccc2nc(-c3cccnc3)nc(Nc3cccc(OC(F)F)c3)c2c1] |
| 235 | 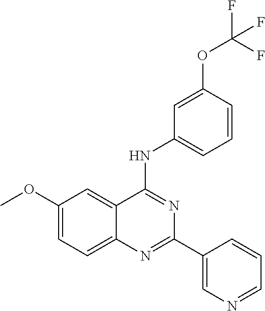 [SMILES: COc1ccc2nc(-c3cccnc3)nc(Nc3cccc(OC(F)(F)F)c3)c2c1] |
| 236 | 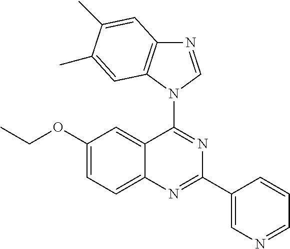 [SMILES: CCOc1ccc2nc(-c3cccnc3)nc(-n3cnc4cc(C)c(C)cc43)c2c1] |
| 237 | 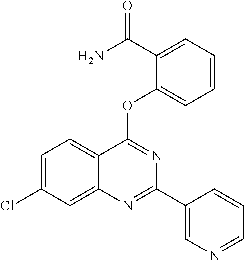 [SMILES: NC(=O)c1ccccc1Oc1nc(-c2cccnc2)nc2cc(Cl)ccc12] |
| 238 | 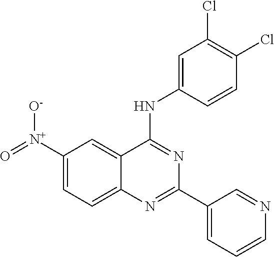 [SMILES: O=[N+]([O-])c1ccc2nc(-c3cccnc3)nc(Nc3ccc(Cl)c(Cl)c3)c2c1] |
| 239 | 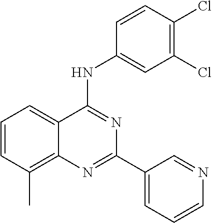 [SMILES: Cc1cccc2c(Nc3ccc(Cl)c(Cl)c3)nc(-c3cccnc3)nc12] |
| 240 | 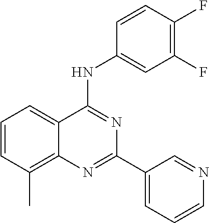 [SMILES: Cc1cccc2c(Nc3ccc(F)c(F)c3)nc(-c3cccnc3)nc12] |
| 241 | 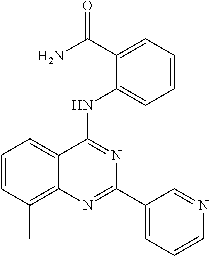 [SMILES: Cc1cccc2c(Nc3ccccc3C(N)=O)nc(-c3cccnc3)nc12] |
| 242 | 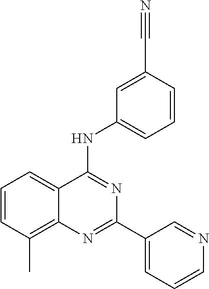 [SMILES: Cc1cccc2c(Nc3cccc(C#N)c3)nc(-c3cccnc3)nc12] |
| 243 | 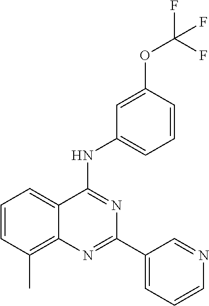 [SMILES: Cc1cccc2c(Nc3cccc(OC(F)(F)F)c3)nc(-c3cccnc3)nc12] |
| 244 | 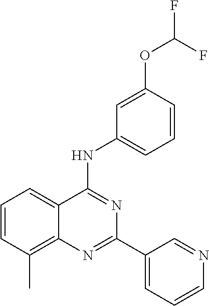 [SMILES: Cc1cccc2c(Nc3cccc(OC(F)F)c3)nc(-c3cccnc3)nc12] |
| 245 | 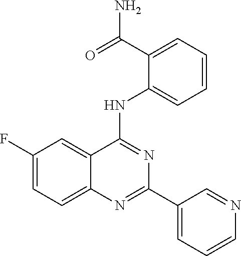 [SMILES: NC(=O)c1ccccc1Nc1nc(-c2cccnc2)nc2ccc(F)cc12] |
| 246 | 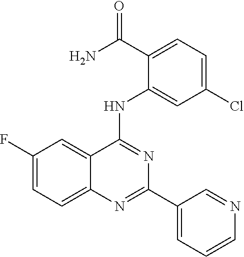 [SMILES: NC(=O)c1ccc(Cl)cc1Nc1nc(-c2cccnc2)nc2ccc(F)cc12] |
| 247 | 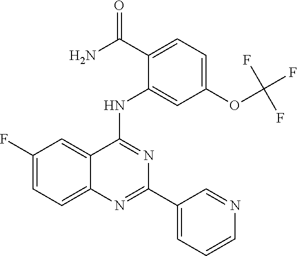 [SMILES: NC(=O)c1ccc(OC(F)(F)F)cc1Nc1nc(-c2cccnc2)nc2ccc(F)cc12] |
| 248 | 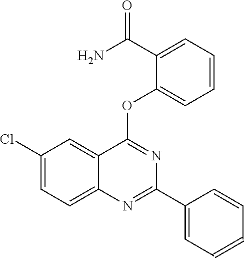 [SMILES: NC(=O)c1ccccc1Oc1nc(-c2ccccc2)nc2ccc(Cl)cc12] |
| 249 | 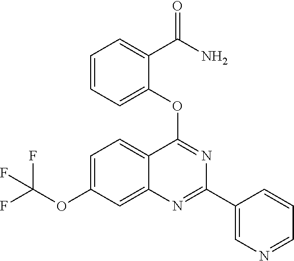 [SMILES: NC(=O)c1ccccc1Oc1nc(-c2cccnc2)nc2cc(OC(F)(F)F)ccc12] |
| 250 | 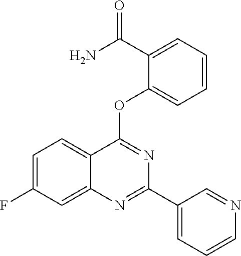 [SMILES: NC(=O)c1ccccc1Oc1nc(-c2cccnc2)nc2cc(F)ccc12] |
| 251 | 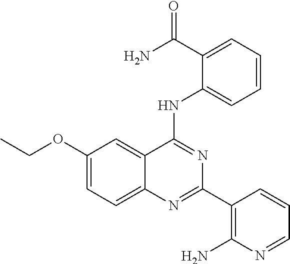 [SMILES: CCOc1ccc2nc(-c3cccnc3N)nc(Nc3ccccc3C(N)=O)c2c1] |
| 252 | 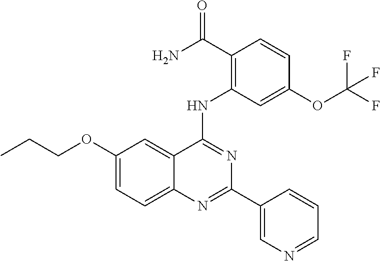 [SMILES: CCCOc1ccc2nc(-c3cccnc3)nc(Nc3cc(OC(F)(F)F)ccc3C(N)=O)c2c1] |
| 253 | 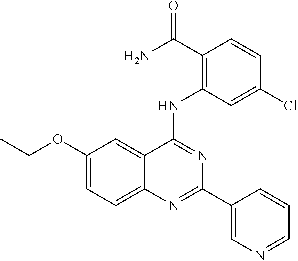 [SMILES: CCOc1ccc2nc(-c3cccnc3)nc(Nc3cc(Cl)ccc3C(N)=O)c2c1] |
| 254 | 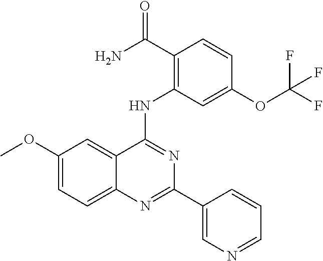 [SMILES: COc1ccc2nc(-c3cccnc3)nc(Nc3cc(OC(F)(F)F)ccc3C(N)=O)c2c1] |
| 255 | 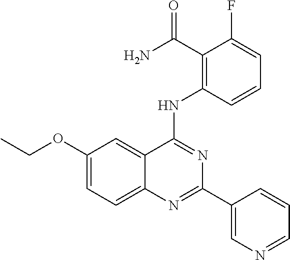 [SMILES: CCOc1ccc2nc(-c3cccnc3)nc(Nc3cccc(F)c3C(N)=O)c2c1] |
| 256 | 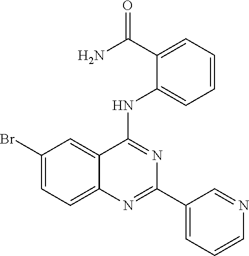 [SMILES: NC(=O)c1ccccc1Nc1nc(-c2cccnc2)nc2ccc(Br)cc12] |
| 257 | 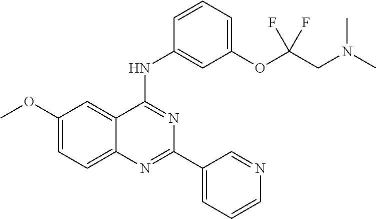 |
| 258 | 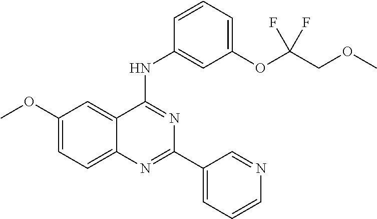 |
| 259 | 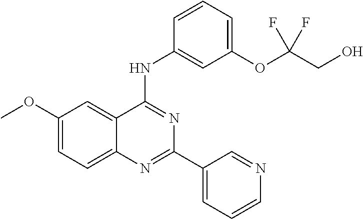 [SMILES: COc1ccc2nc(-c3cccnc3)nc(Nc3cccc(OC(F)(F)CO)c3)c2c1] |
| 260 | 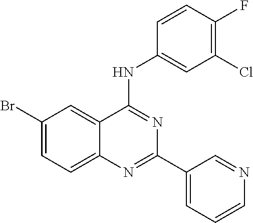 [SMILES: Fc1ccc(Nc2nc(-c3cccnc3)nc3ccc(Br)cc23)cc1Cl] |
| 261 | 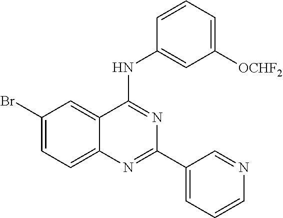 [SMILES: FC(F)Oc1cccc(Nc2nc(-c3cccnc3)nc3ccc(Br)cc23)c1] |
| 262 | 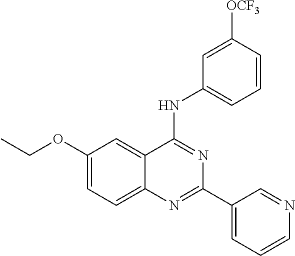 [SMILES: CCOc1ccc2nc(-c3cccnc3)nc(Nc3cccc(OC(F)(F)F)c3)c2c1] |
| 263 | 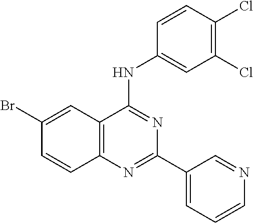 [SMILES: Clc1ccc(Nc2nc(-c3cccnc3)nc3ccc(Br)cc23)cc1Cl] |
| 264 | 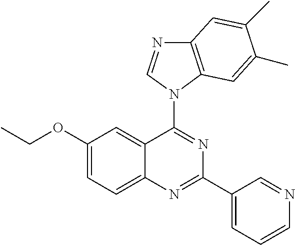 [SMILES: CCOc1ccc2nc(-c3cccnc3)nc(-n3cnc4cc(C)c(C)cc43)c2c1] |
| 265 | 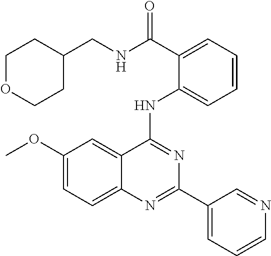 [SMILES: COc1ccc2nc(-c3cccnc3)nc(Nc3ccccc3C(=O)NCC3CCOCC3)c2c1] |
| 266 | 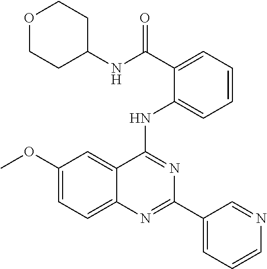 [SMILES: COc1ccc2nc(-c3cccnc3)nc(Nc3ccccc3C(=O)NC3CCOCC3)c2c1] |
| 267 | 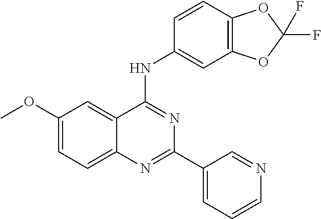 [SMILES: COc1ccc2nc(-c3cccnc3)nc(Nc3ccc4c(c3)OC(F)(F)O4)c2c1] |
| 268 | 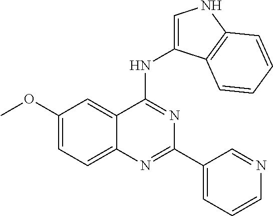 [SMILES: COc1ccc2nc(-c3cccnc3)nc(Nc3c[nH]c4ccccc34)c2c1] |
| 269 | 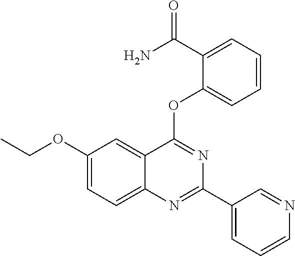 [SMILES: CCOc1ccc2nc(-c3cccnc3)nc(Oc3ccccc3C(N)=O)c2c1] |
| 270 | 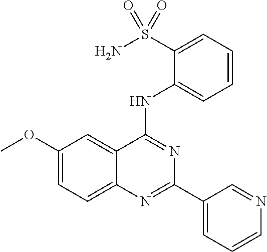 [SMILES: COc1ccc2nc(-c3cccnc3)nc(Nc3ccccc3S(N)(=O)=O)c2c1] |
| 271 | 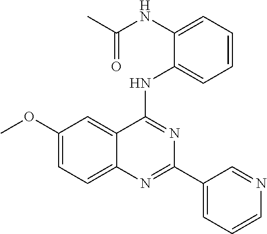 [SMILES: COc1ccc2nc(-c3cccnc3)nc(Nc3ccccc3NC(C)=O)c2c1] |
| 272 | 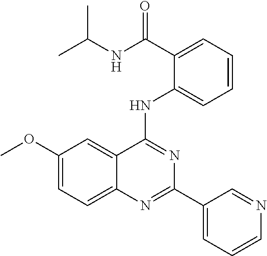 [SMILES: COc1ccc2nc(-c3cccnc3)nc(Nc3ccccc3C(=O)NC(C)C)c2c1] |
| 273 | 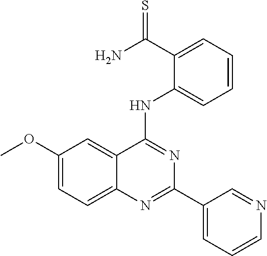 [SMILES: COc1ccc2nc(-c3cccnc3)nc(Nc3ccccc3C(N)=S)c2c1] |
| 274 | 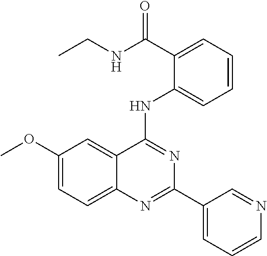 [SMILES: CCNC(=O)c1ccccc1Nc1nc(-c2cccnc2)nc2ccc(OC)cc12] |
| 275 | 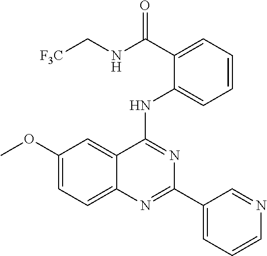 [SMILES: COc1ccc2nc(-c3cccnc3)nc(Nc3ccccc3C(=O)NCC(F)(F)F)c2c1] |
| 276 | 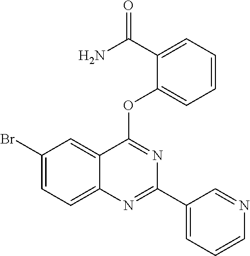 [SMILES: NC(=O)c1ccccc1Oc1nc(-c2cccnc2)nc2ccc(Br)cc12] |
| 277 | 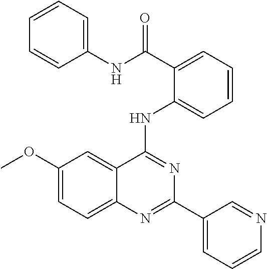 [SMILES: COc1ccc2nc(-c3cccnc3)nc(Nc3ccccc3C(=O)Nc3ccccc3)c2c1] |
| 278 | 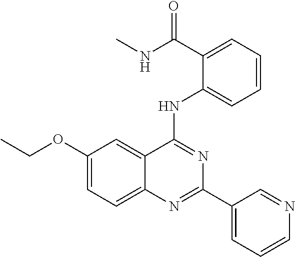 [SMILES: CCOc1ccc2nc(-c3cccnc3)nc(Nc3ccccc3C(=O)NC)c2c1] |
| 279 | 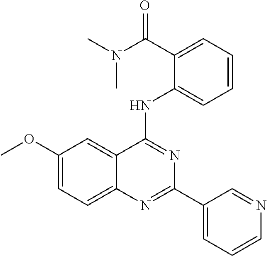 [SMILES: COc1ccc2nc(-c3cccnc3)nc(Nc3ccccc3C(=O)N(C)C)c2c1] |
| 280 | 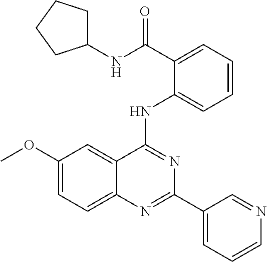 [SMILES: COc1ccc2nc(-c3cccnc3)nc(Nc3ccccc3C(=O)NC3CCCC3)c2c1] |
| 281 | 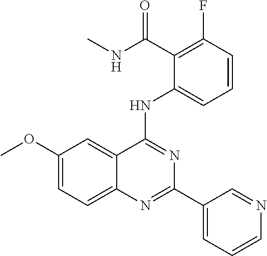 [SMILES: CNC(=O)c1c(F)cccc1Nc1nc(-c2cccnc2)nc2ccc(OC)cc12] |
| 282 | 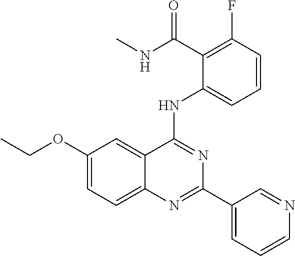 [SMILES: CCOc1ccc2nc(-c3cccnc3)nc(Nc3cccc(F)c3C(=O)NC)c2c1] |
| 283 | 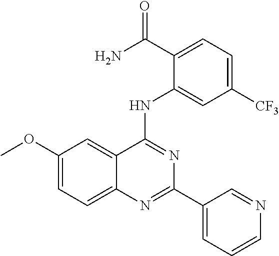 [SMILES: COc1ccc2nc(-c3cccnc3)nc(Nc3cc(C(F)(F)F)ccc3C(N)=O)c2c1] |
| 284 | 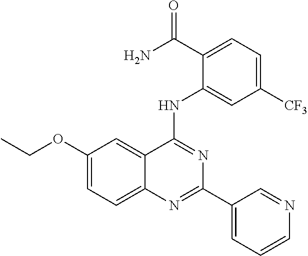 [SMILES: CCOc1ccc2nc(-c3cccnc3)nc(Nc3cc(C(F)(F)F)ccc3C(N)=O)c2c1] |
| 285 | 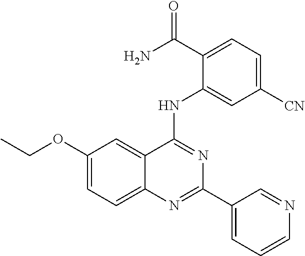 [SMILES: CCOc1ccc2nc(-c3cccnc3)nc(Nc3cc(C#N)ccc3C(N)=O)c2c1] |
| 286 | 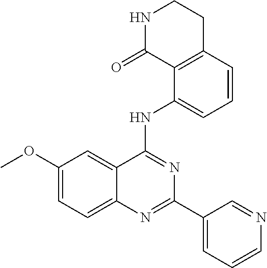 [SMILES: COc1ccc2nc(-c3cccnc3)nc(Nc3cccc4c3C(=O)NCC4)c2c1] |
| 287 | 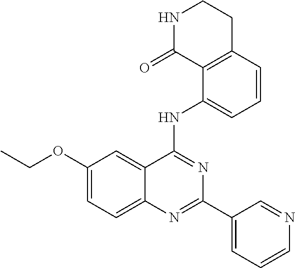 [SMILES: CCOc1ccc2nc(-c3cccnc3)nc(Nc3cccc4c3C(=O)NCC4)c2c1] |
| 288 | 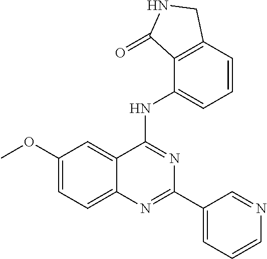 [SMILES: COc1ccc2nc(-c3cccnc3)nc(Nc3cccc4c3C(=O)NC4)c2c1] |
| 289 | 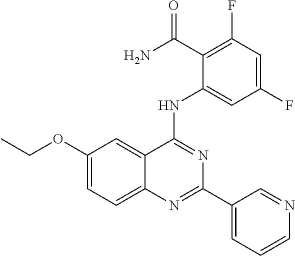 [SMILES: CCOc1ccc2nc(-c3cccnc3)nc(Nc3cc(F)cc(F)c3C(N)=O)c2c1] |
| 290 | 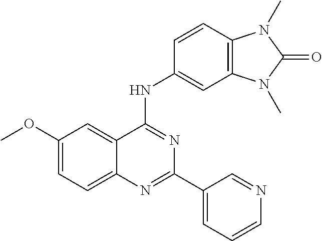 [SMILES: COc1ccc2nc(-c3cccnc3)nc(Nc3ccc4c(c3)n(C)c(=O)n4C)c2c1] |
| 291 | 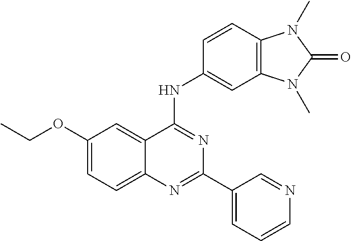 [SMILES: CCOc1ccc2nc(-c3cccnc3)nc(Nc3ccc4c(c3)n(C)c(=O)n4C)c2c1] |
| 292 | 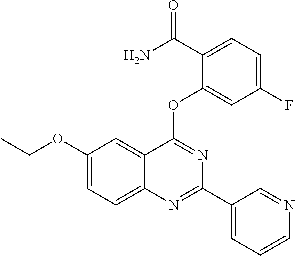 [SMILES: CCOc1ccc2nc(-c3cccnc3)nc(Oc3cc(F)ccc3C(N)=O)c2c1] |
| 293 | 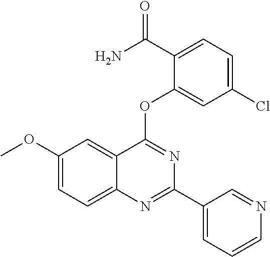 [SMILES: COc1ccc2nc(-c3cccnc3)nc(Oc3cc(Cl)ccc3C(N)=O)c2c1] |
| 294 | 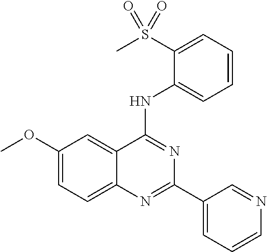 [SMILES: COc1ccc2nc(-c3cccnc3)nc(Nc3ccccc3S(C)(=O)=O)c2c1] |
| 295 | 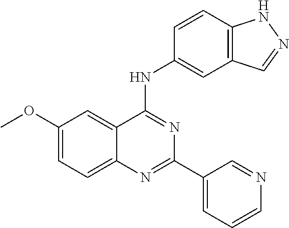 [SMILES: COc1ccc2nc(-c3cccnc3)nc(Nc3ccc4[nH]ncc4c3)c2c1] |
| 296 | 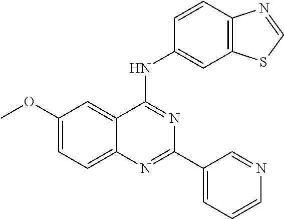 [SMILES: COc1ccc2nc(-c3cccnc3)nc(Nc3ccc4ncsc4c3)c2c1] |
| 297 | 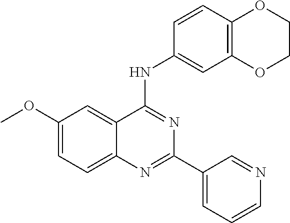 [SMILES: COc1ccc2nc(-c3cccnc3)nc(Nc3ccc4c(c3)OCCO4)c2c1] |
| 298 | 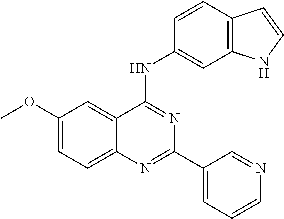 [SMILES: COc1ccc2nc(-c3cccnc3)nc(Nc3ccc4cc[nH]c4c3)c2c1] |
| 299 | 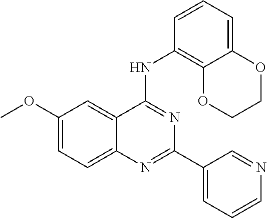 [SMILES: COc1ccc2nc(-c3cccnc3)nc(Nc3cccc4c3OCCO4)c2c1] |
| 300 | 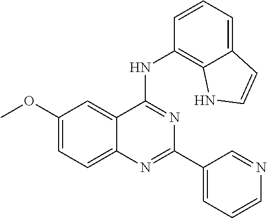 [SMILES: COc1ccc2nc(-c3cccnc3)nc(Nc3cccc4cc[nH]c34)c2c1] |
| 301 | 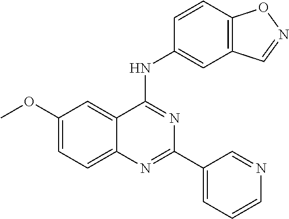 [SMILES: COc1ccc2nc(-c3cccnc3)nc(Nc3ccc4oncc4c3)c2c1] |
| 302 | 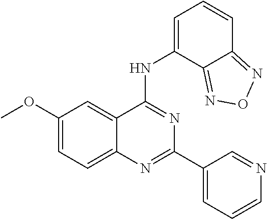 [SMILES: COc1ccc2nc(-c3cccnc3)nc(Nc3cccc4nonc34)c2c1] |
| 303 | 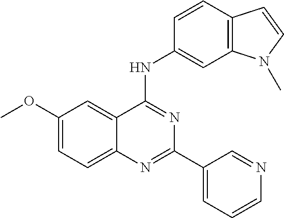 [SMILES: COc1ccc2nc(-c3cccnc3)nc(Nc3ccc4ccn(C)c4c3)c2c1] |
| 304 | 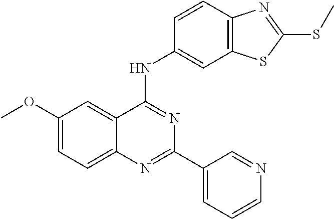 |
| 305 | 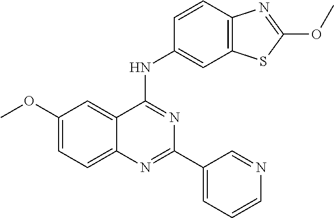 |
| 306 | 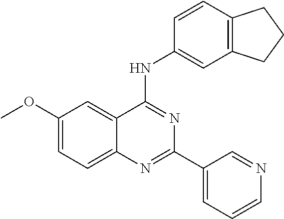 [SMILES: COc1ccc2nc(-c3cccnc3)nc(Nc3ccc4c(c3)CCC4)c2c1] |
| 307 | 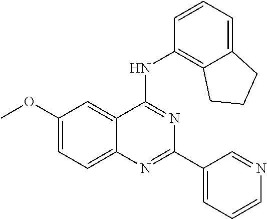 [SMILES: COc1ccc2nc(-c3cccnc3)nc(Nc3cccc4c3CCC4)c2c1] |
| 308 | 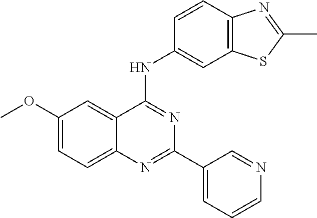 |
| 309 | 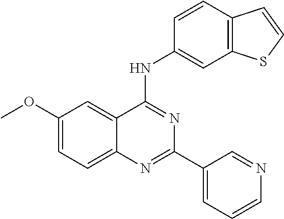 [SMILES: COc1ccc2nc(-c3cccnc3)nc(Nc3ccc4ccsc4c3)c2c1] |
| 310 | 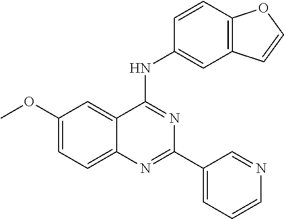 [SMILES: COc1ccc2nc(-c3cccnc3)nc(Nc3ccc4occc4c3)c2c1] |
| 311 | 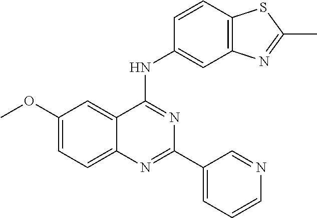 |
| 312 | 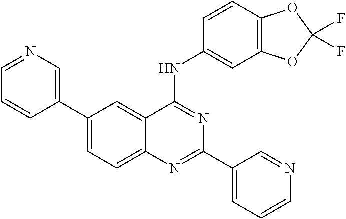 [SMILES: FC1(F)Oc2ccc(Nc3nc(-c4cccnc4)nc4ccc(-c5cccnc5)cc34)cc2O1] |
| 313 | 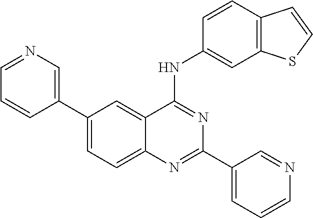 [SMILES: c1cncc(-c2ccc3nc(-c4cccnc4)nc(Nc4ccc5ccsc5c4)c3c2)c1] |
| 314 | 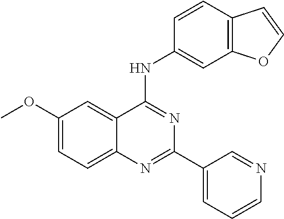 [SMILES: COc1ccc2nc(-c3cccnc3)nc(Nc3ccc4ccoc4c3)c2c1] |
| 315 | 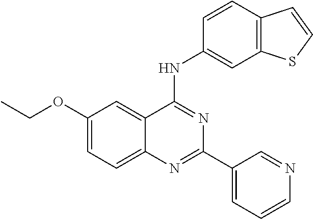 [SMILES: CCOc1ccc2nc(-c3cccnc3)nc(Nc3ccc4ccsc4c3)c2c1] |
| 316 | 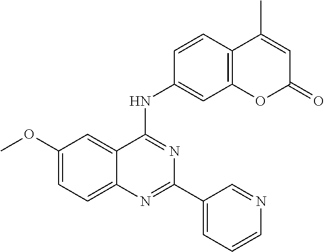 [SMILES: COc1ccc2nc(-c3cccnc3)nc(Nc3ccc4c(C)cc(=O)oc4c3)c2c1] |
| 317 | 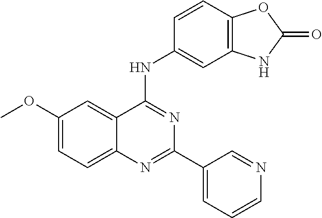 [SMILES: COc1ccc2nc(-c3cccnc3)nc(Nc3ccc4oc(=O)[nH]c4c3)c2c1] |
| 318 | 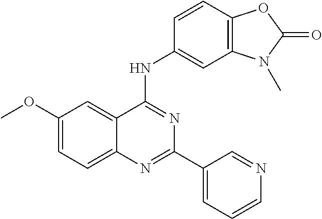 [SMILES: COc1ccc2nc(-c3cccnc3)nc(Nc3ccc4oc(=O)n(C)c4c3)c2c1] |
| 319 | 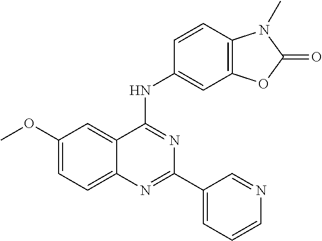 [SMILES: COc1ccc2nc(-c3cccnc3)nc(Nc3ccc4c(c3)oc(=O)n4C)c2c1] |
| 320 | 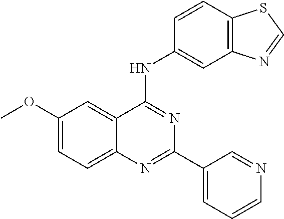 [SMILES: COc1ccc2nc(-c3cccnc3)nc(Nc3ccc4scnc4c3)c2c1] |
| 321 | 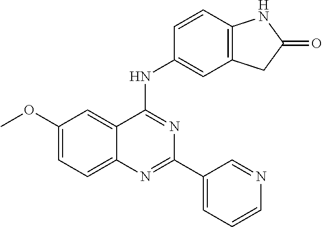 [SMILES: COc1ccc2nc(-c3cccnc3)nc(Nc3ccc4c(c3)CC(=O)N4)c2c1] |
| 322 | 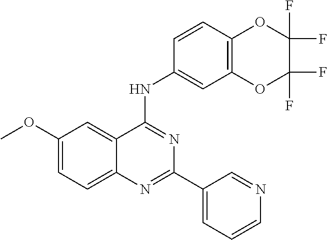 [SMILES: COc1ccc2nc(-c3cccnc3)nc(Nc3ccc4c(c3)OC(F)(F)C(F)(F)O4)c2c1] |
| 323 | 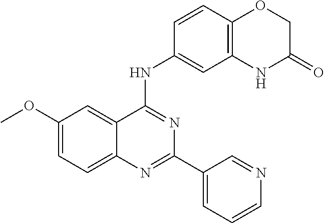 [SMILES: COc1ccc2nc(-c3cccnc3)nc(Nc3ccc4c(c3)NC(=O)CO4)c2c1] |
| 324 | 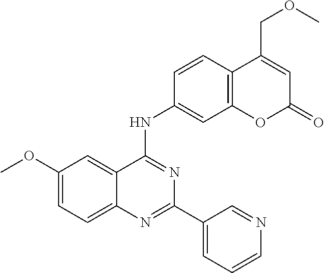 [SMILES: COCc1cc(=O)oc2cc(Nc3nc(-c4cccnc4)nc4ccc(OC)cc34)ccc12] |
| 325 | 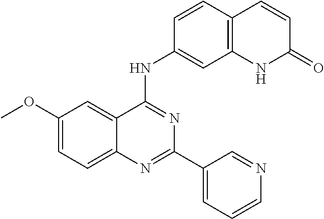 [SMILES: COc1ccc2nc(-c3cccnc3)nc(Nc3ccc4ccc(=O)[nH]c4c3)c2c1] |
| 326 | 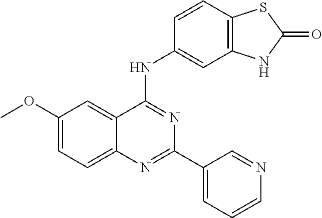 [SMILES: COc1ccc2nc(-c3cccnc3)nc(Nc3ccc4sc(=O)[nH]c4c3)c2c1] |
| 327 | 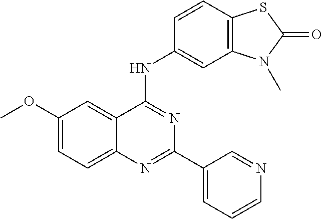 [SMILES: COc1ccc2nc(-c3cccnc3)nc(Nc3ccc4sc(=O)n(C)c4c3)c2c1] |
| 328 | 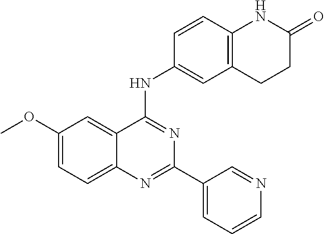 [SMILES: COc1ccc2nc(-c3cccnc3)nc(Nc3ccc4c(c3)CCC(=O)N4)c2c1] |
| 329 | 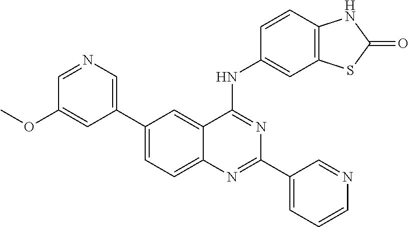 [SMILES: COc1cncc(-c2ccc3nc(-c4cccnc4)nc(Nc4ccc5[nH]c(=O)sc5c4)c3c2)c1] |
| 330 | 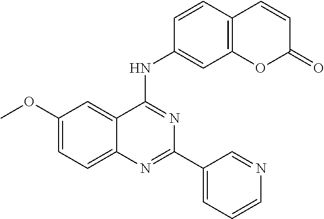 [SMILES: COc1ccc2nc(-c3cccnc3)nc(Nc3ccc4ccc(=O)oc4c3)c2c1] |
| 331 | 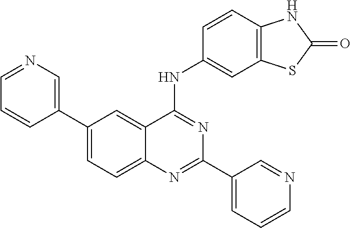 [SMILES: O=c1[nH]c2ccc(Nc3nc(-c4cccnc4)nc4ccc(-c5cccnc5)cc34)cc2s1] |
| 332 | 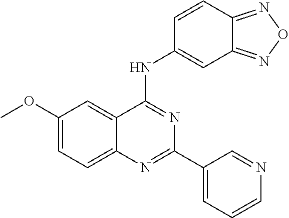 [SMILES: COc1ccc2nc(-c3cccnc3)nc(Nc3ccc4nonc4c3)c2c1] |
| 333 | 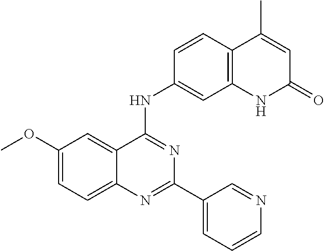 [SMILES: COc1ccc2nc(-c3cccnc3)nc(Nc3ccc4c(C)cc(=O)[nH]c4c3)c2c1] |
| 334 | 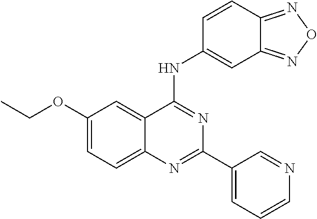 [SMILES: CCOc1ccc2nc(-c3cccnc3)nc(Nc3ccc4nonc4c3)c2c1] |
| 335 | 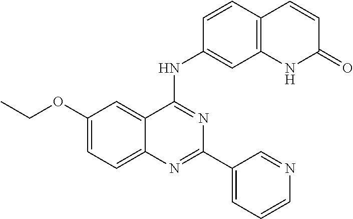 [SMILES: CCOc1ccc2nc(-c3cccnc3)nc(Nc3ccc4ccc(=O)[nH]c4c3)c2c1] |
| 336 | 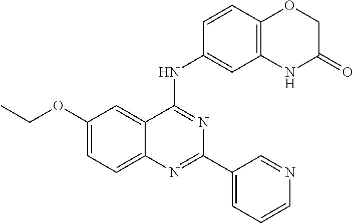 [SMILES: CCOc1ccc2nc(-c3cccnc3)nc(Nc3ccc4c(c3)NC(=O)CO4)c2c1] |
| 337 | 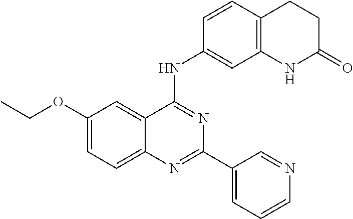 [SMILES: CCOc1ccc2nc(-c3cccnc3)nc(Nc3ccc4c(c3)NC(=O)CC4)c2c1] |
| 338 | 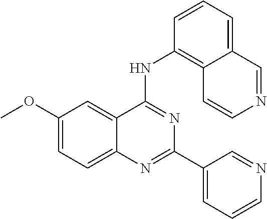 [SMILES: COc1ccc2nc(-c3cccnc3)nc(Nc3cccc4cnccc34)c2c1] |
| 339 | 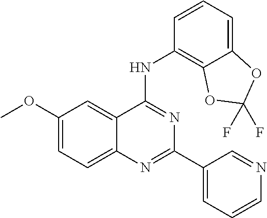 [SMILES: COc1ccc2nc(-c3cccnc3)nc(Nc3cccc4c3OC(F)(F)O4)c2c1] |
| 340 | 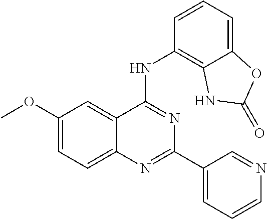 [SMILES: COc1ccc2nc(-c3cccnc3)nc(Nc3cccc4oc(=O)[nH]c34)c2c1] |
| 341 | 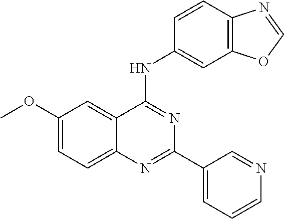 [SMILES: COc1ccc2nc(-c3cccnc3)nc(Nc3ccc4ncoc4c3)c2c1] |
| 342 | 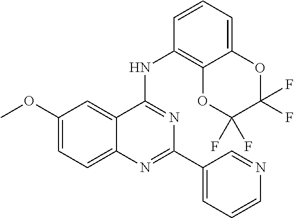 [SMILES: COc1ccc2nc(-c3cccnc3)nc(Nc3cccc4c3OC(F)(F)C(F)(F)O4)c2c1] |
| 343 | 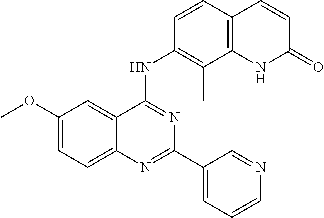 [SMILES: COc1ccc2nc(-c3cccnc3)nc(Nc3ccc4ccc(=O)[nH]c4c3C)c2c1] |
| 344 | 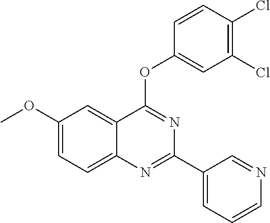 [SMILES: COc1ccc2nc(-c3cccnc3)nc(Oc3ccc(Cl)c(Cl)c3)c2c1] |
| 345 | 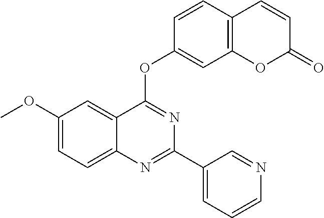 [SMILES: COc1ccc2nc(-c3cccnc3)nc(Oc3ccc4ccc(=O)oc4c3)c2c1] |
| 346 | 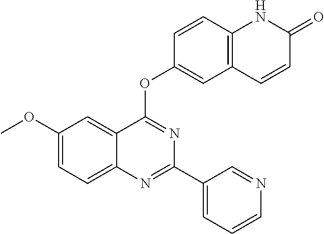 [SMILES: COc1ccc2nc(-c3cccnc3)nc(Oc3ccc4[nH]c(=O)ccc4c3)c2c1] |
| 347 | 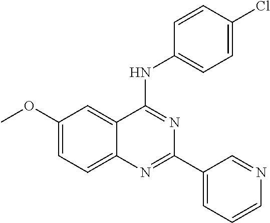 [SMILES: COc1ccc2nc(-c3cccnc3)nc(Nc3ccc(Cl)cc3)c2c1] |
| 348 | 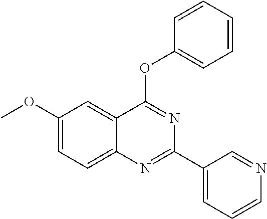 [SMILES: COc1ccc2nc(-c3cccnc3)nc(Oc3ccccc3)c2c1] |
| 349 | 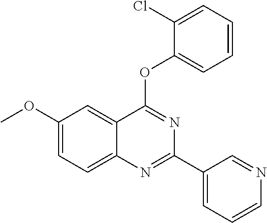 [SMILES: COc1ccc2nc(-c3cccnc3)nc(Oc3ccccc3Cl)c2c1] |
| 350 | 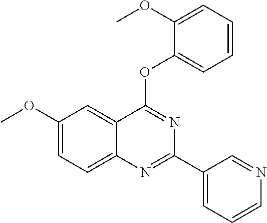 [SMILES: COc1ccc2nc(-c3cccnc3)nc(Oc3ccccc3OC)c2c1] |
| 351 | 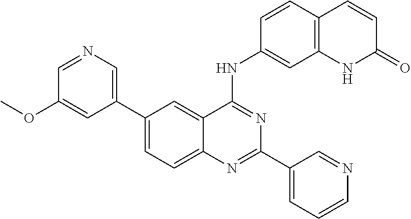 [SMILES: COc1cncc(-c2ccc3nc(-c4cccnc4)nc(Nc4ccc5ccc(=O)[nH]c5c4)c3c2)c1] |
| 352 | 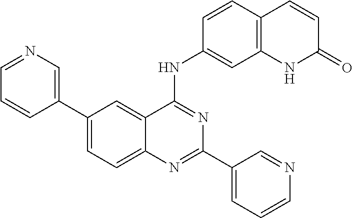 [SMILES: O=c1ccc2ccc(Nc3nc(-c4cccnc4)nc4ccc(-c5cccnc5)cc34)cc2[nH]1] |
| 353 | 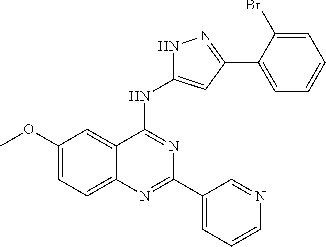 [SMILES: COc1ccc2nc(-c3cccnc3)nc(Nc3cc(-c4ccccc4Br)n[nH]3)c2c1] |
| 354 | 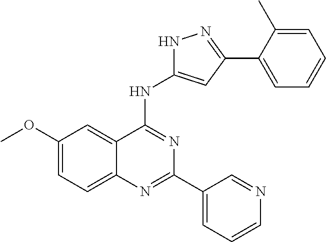 [SMILES: COc1ccc2nc(-c3cccnc3)nc(Nc3cc(-c4ccccc4C)n[nH]3)c2c1] |
| 355 | 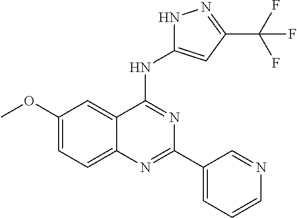 [SMILES: COc1ccc2nc(-c3cccnc3)nc(Nc3cc(C(F)(F)F)n[nH]3)c2c1] |
| 356 | 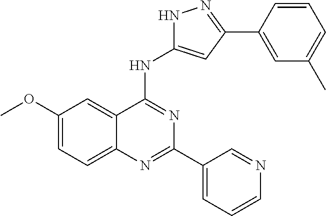 |
| 357 | 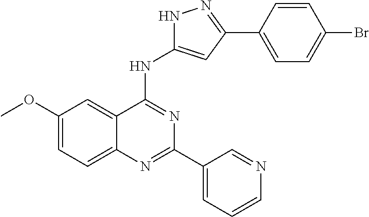 [SMILES: COc1ccc2nc(-c3cccnc3)nc(Nc3cc(-c4ccc(Br)cc4)n[nH]3)c2c1] |
| 358 | 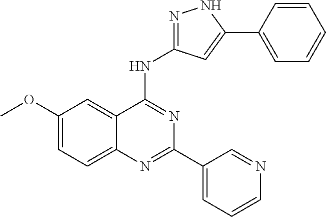 [SMILES: COc1ccc2nc(-c3cccnc3)nc(Nc3cc(-c4ccccc4)[nH]n3)c2c1] |
| 359 | 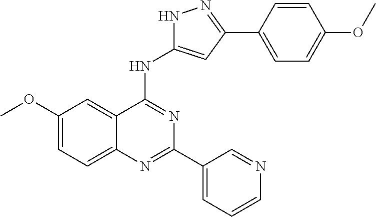 |
| 360 | 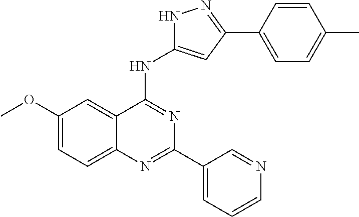 |
| 361 | 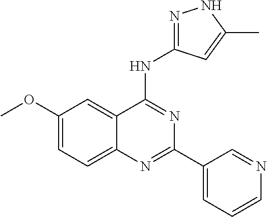 [SMILES: COc1ccc2nc(-c3cccnc3)nc(Nc3cc(C)[nH]n3)c2c1] |
| 362 | 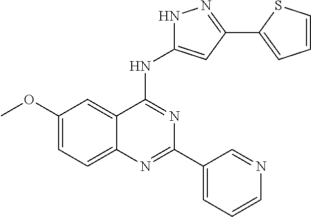 [SMILES: COc1ccc2nc(-c3cccnc3)nc(Nc3cc(-c4cccs4)n[nH]3)c2c1] |
| 363 | 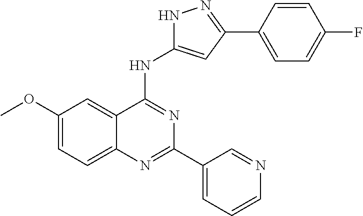 [SMILES: COc1ccc2nc(-c3cccnc3)nc(Nc3cc(-c4ccc(F)cc4)n[nH]3)c2c1] |
| 364 | 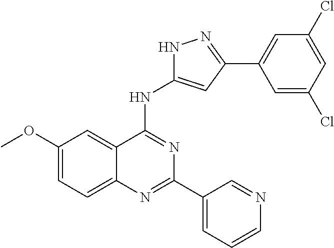 [SMILES: COc1ccc2nc(-c3cccnc3)nc(Nc3cc(-c4cc(Cl)cc(Cl)c4)n[nH]3)c2c1] |
| 365 | 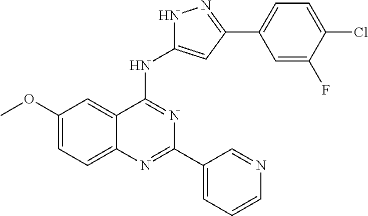 [SMILES: COc1ccc2nc(-c3cccnc3)nc(Nc3cc(-c4ccc(Cl)c(F)c4)n[nH]3)c2c1] |
| 366 | 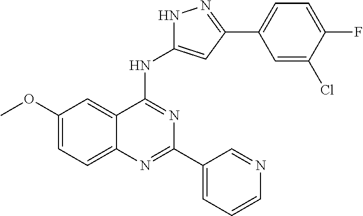 [SMILES: COc1ccc2nc(-c3cccnc3)nc(Nc3cc(-c4ccc(F)c(Cl)c4)n[nH]3)c2c1] |
| 367 | 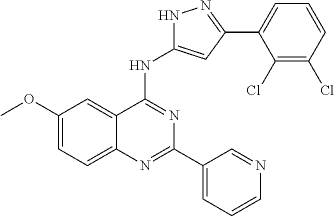 [SMILES: COc1ccc2nc(-c3cccnc3)nc(Nc3cc(-c4cccc(Cl)c4Cl)n[nH]3)c2c1] |
| 368 | 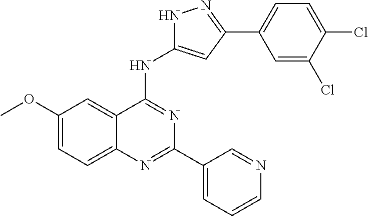 [SMILES: COc1ccc2nc(-c3cccnc3)nc(Nc3cc(-c4ccc(Cl)c(Cl)c4)n[nH]3)c2c1] |
| 369 | 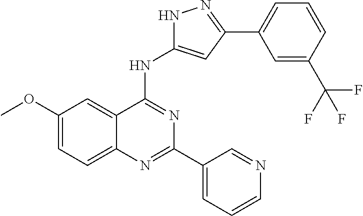 [SMILES: COc1ccc2nc(-c3cccnc3)nc(Nc3cc(-c4cccc(C(F)(F)F)c4)n[nH]3)c2c1] |
| 370 | 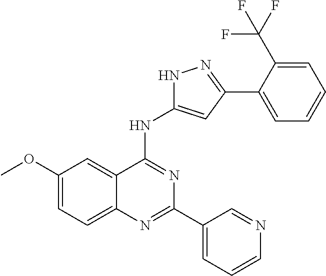 [SMILES: COc1ccc2nc(-c3cccnc3)nc(Nc3cc(-c4ccccc4C(F)(F)F)n[nH]3)c2c1] |
| 371 | 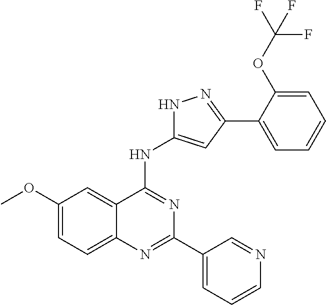 [SMILES: COc1ccc2nc(-c3cccnc3)nc(Nc3cc(-c4ccccc4OC(F)(F)F)n[nH]3)c2c1] |
| 372 | 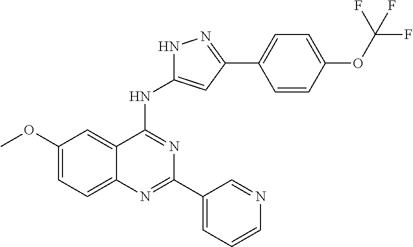 [SMILES: COc1ccc2nc(-c3cccnc3)nc(Nc3cc(-c4ccc(OC(F)(F)F)cc4)n[nH]3)c2c1] |
| 373 | 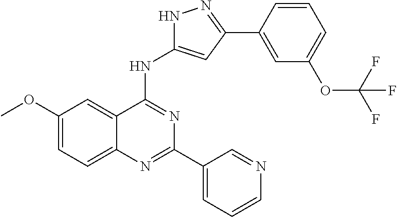 [SMILES: COc1ccc2nc(-c3cccnc3)nc(Nc3cc(-c4cccc(OC(F)(F)F)c4)n[nH]3)c2c1] |
| 374 | 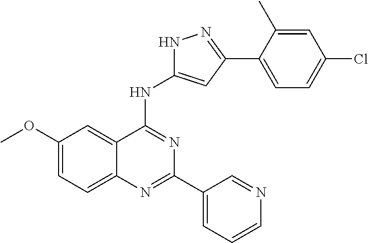 [SMILES: COc1ccc2nc(-c3cccnc3)nc(Nc3cc(-c4ccc(Cl)cc4C)n[nH]3)c2c1] |
| 375 | 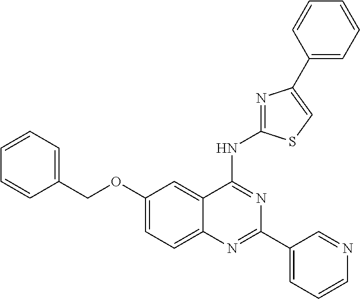 [SMILES: c1ccc(COc2ccc3nc(-c4cccnc4)nc(Nc4nc(-c5ccccc5)cs4)c3c2)cc1] |
| 376 | 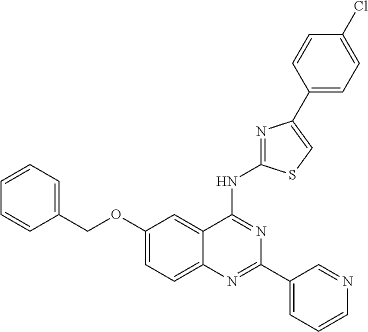 [SMILES: Clc1ccc(-c2csc(Nc3nc(-c4cccnc4)nc4ccc(OCc5ccccc5)cc34)n2)cc1] |
| 377 | 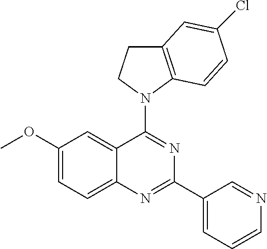 [SMILES: COc1ccc2nc(-c3cccnc3)nc(N3CCc4cc(Cl)ccc43)c2c1] |
| 378 | 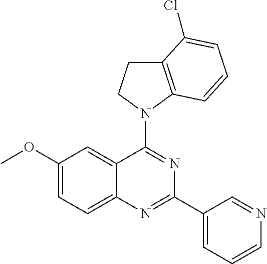 [SMILES: COc1ccc2nc(-c3cccnc3)nc(N3CCc4c(Cl)cccc43)c2c1] |
| 379 | 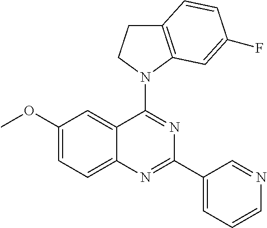 [SMILES: COc1ccc2nc(-c3cccnc3)nc(N3CCc4ccc(F)cc43)c2c1] |
| 380 | 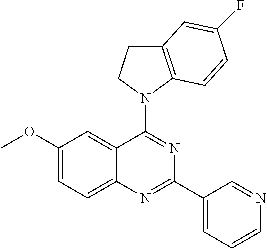 [SMILES: COc1ccc2nc(-c3cccnc3)nc(N3CCc4cc(F)ccc43)c2c1] |
| 381 | 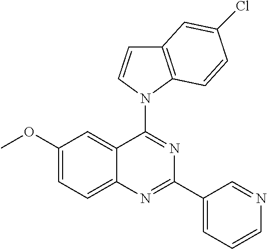 [SMILES: COc1ccc2nc(-c3cccnc3)nc(-n3ccc4cc(Cl)ccc43)c2c1] |
| 382 | 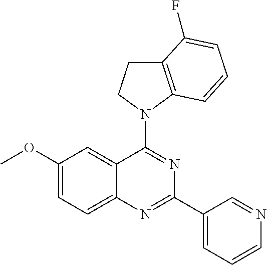 [SMILES: COc1ccc2nc(-c3cccnc3)nc(N3CCc4c(F)cccc43)c2c1] |
| 383 | 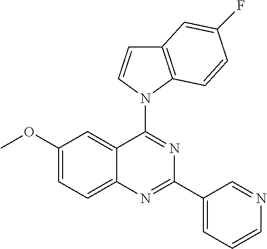 [SMILES: COc1ccc2nc(-c3cccnc3)nc(-n3ccc4cc(F)ccc43)c2c1] |
| 384 | 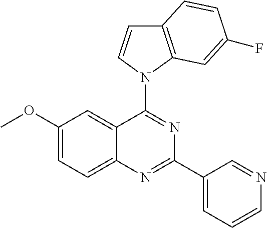 [SMILES: COc1ccc2nc(-c3cccnc3)nc(-n3ccc4ccc(F)cc43)c2c1] |
| 385 | 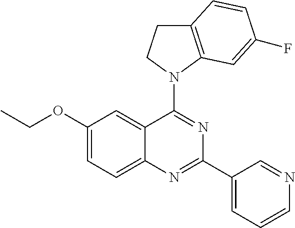 [SMILES: CCOc1ccc2nc(-c3cccnc3)nc(N3CCc4ccc(F)cc43)c2c1] |
| 386 | 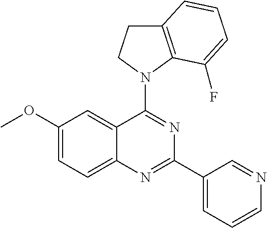 [SMILES: COc1ccc2nc(-c3cccnc3)nc(N3CCc4cccc(F)c43)c2c1] |
| 387 | 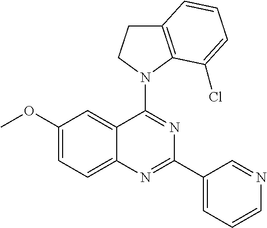 [SMILES: COc1ccc2nc(-c3cccnc3)nc(N3CCc4cccc(Cl)c43)c2c1] |
| 388 | 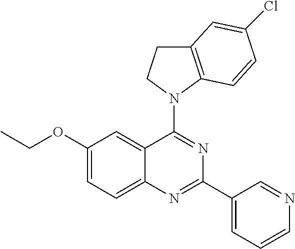 [SMILES: CCOc1ccc2nc(-c3cccnc3)nc(N3CCc4cc(Cl)ccc43)c2c1] |
| 389 | 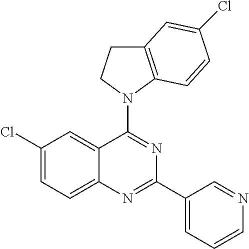 [SMILES: Clc1ccc2c(c1)CCN2c1nc(-c2cccnc2)nc2ccc(Cl)cc12] |
| 390 | 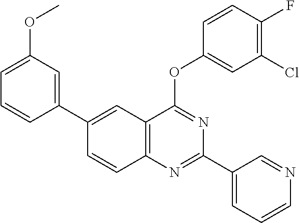 [SMILES: COc1cccc(-c2ccc3nc(-c4cccnc4)nc(Oc4ccc(F)c(Cl)c4)c3c2)c1] |
| 391 | 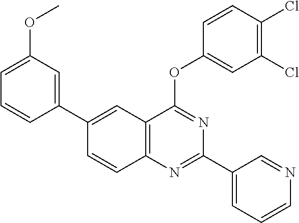 [SMILES: COc1cccc(-c2ccc3nc(-c4cccnc4)nc(Oc4ccc(Cl)c(Cl)c4)c3c2)c1] |
| 392 | 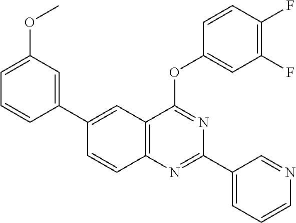 [SMILES: COc1cccc(-c2ccc3nc(-c4cccnc4)nc(Oc4ccc(F)c(F)c4)c3c2)c1] |
| 393 | 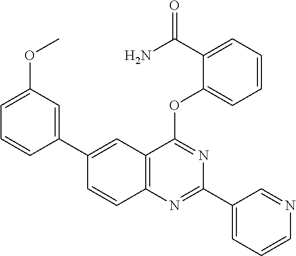 [SMILES: COc1cccc(-c2ccc3nc(-c4cccnc4)nc(Oc4ccccc4C(N)=O)c3c2)c1] |
| 394 | 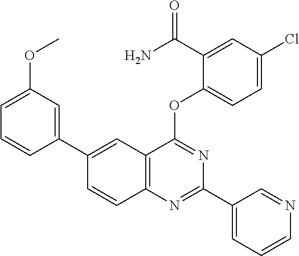 [SMILES: COc1cccc(-c2ccc3nc(-c4cccnc4)nc(Oc4ccc(Cl)cc4C(N)=O)c3c2)c1] |
| 395 | 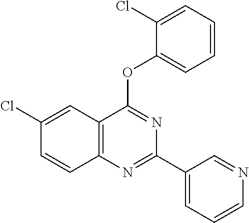 [SMILES: Clc1ccc2nc(-c3cccnc3)nc(Oc3ccccc3Cl)c2c1] |
| 396 | 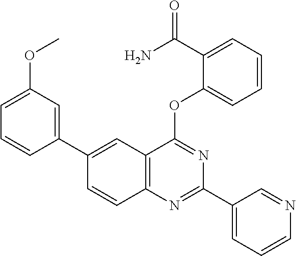 [SMILES: COc1cccc(-c2ccc3nc(-c4cccnc4)nc(Oc4ccccc4C(N)=O)c3c2)c1] |
| 397 | 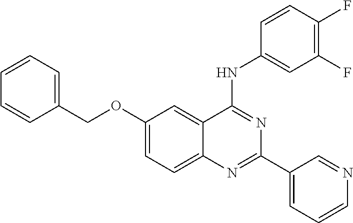 [SMILES: Fc1ccc(Nc2nc(-c3cccnc3)nc3ccc(OCc4ccccc4)cc23)cc1F] |
| 398 | 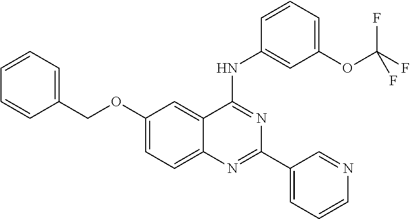 [SMILES: FC(F)(F)Oc1cccc(Nc2nc(-c3cccnc3)nc3ccc(OCc4ccccc4)cc23)c1] |
| 399 | 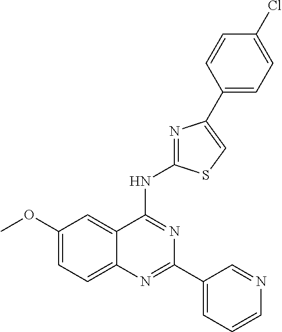 [SMILES: COc1ccc2nc(-c3cccnc3)nc(Nc3nc(-c4ccc(Cl)cc4)cs3)c2c1] |
| 400 | 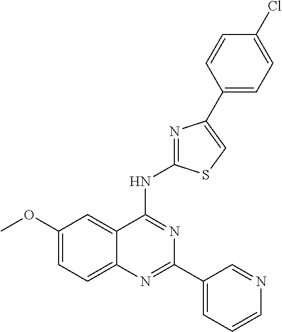 [SMILES: COc1ccc2nc(-c3cccnc3)nc(Nc3nc(-c4ccc(Cl)cc4)cs3)c2c1] |
| 401 | 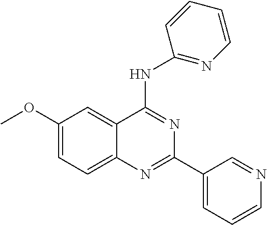 [SMILES: COc1ccc2nc(-c3cccnc3)nc(Nc3ccccn3)c2c1] |
| 402 | 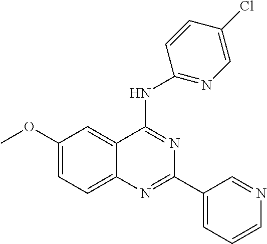 [SMILES: COc1ccc2nc(-c3cccnc3)nc(Nc3ccc(Cl)cn3)c2c1] |
| 403 | 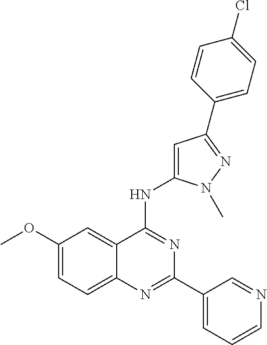 [SMILES: COc1ccc2nc(-c3cccnc3)nc(Nc3cc(-c4ccc(Cl)cc4)nn3C)c2c1] |
| 404 | 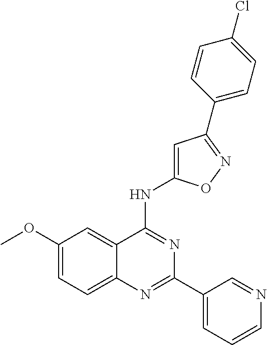 [SMILES: COc1ccc2nc(-c3cccnc3)nc(Nc3cc(-c4ccc(Cl)cc4)no3)c2c1] |
| 405 | 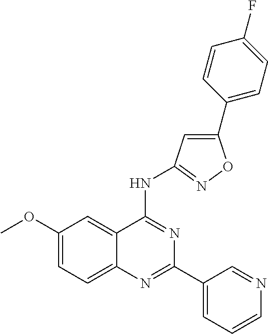 [SMILES: COc1ccc2nc(-c3cccnc3)nc(Nc3cc(-c4ccc(F)cc4)on3)c2c1] |
| 406 | 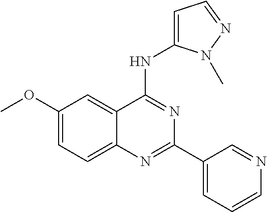 [SMILES: COc1ccc2nc(-c3cccnc3)nc(Nc3ccnn3C)c2c1] |
| 407 | 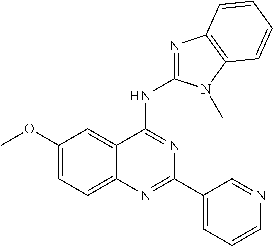 [SMILES: COc1ccc2nc(-c3cccnc3)nc(Nc3nc4ccccc4n3C)c2c1] |
| 408 | 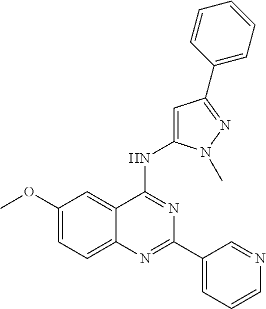 [SMILES: COc1ccc2nc(-c3cccnc3)nc(Nc3cc(-c4ccccc4)nn3C)c2c1] |
| 409 | 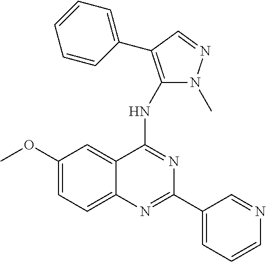 [SMILES: COc1ccc2nc(-c3cccnc3)nc(Nc3c(-c4ccccc4)cnn3C)c2c1] |
| 410 | 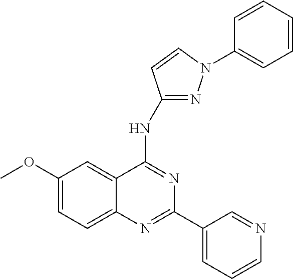 [SMILES: COc1ccc2nc(-c3cccnc3)nc(Nc3ccn(-c4ccccc4)n3)c2c1] |
| 411 | 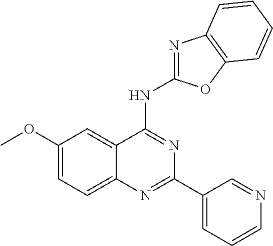 [SMILES: COc1ccc2nc(-c3cccnc3)nc(Nc3nc4ccccc4o3)c2c1] |
| 412 | 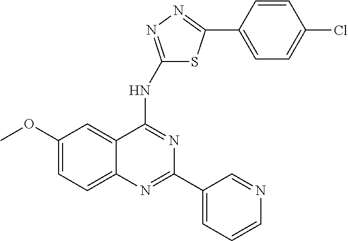 [SMILES: COc1ccc2nc(-c3cccnc3)nc(Nc3nnc(-c4ccc(Cl)cc4)s3)c2c1] |
| 413 | 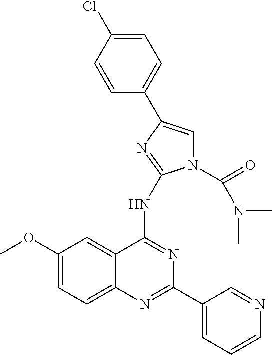 |
| 414 | 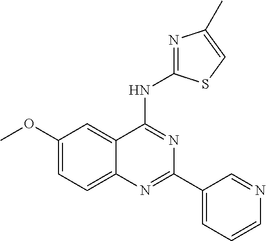 [SMILES: COc1ccc2nc(-c3cccnc3)nc(Nc3nc(C)cs3)c2c1] |
| 415 | 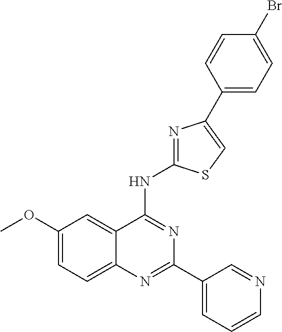 [SMILES: COc1ccc2nc(-c3cccnc3)nc(Nc3nc(-c4ccc(Br)cc4)cs3)c2c1] |
| 416 | 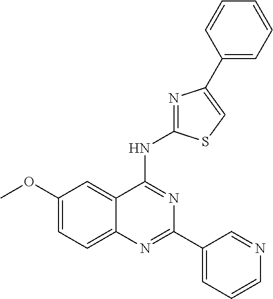 [SMILES: COc1ccc2nc(-c3cccnc3)nc(Nc3nc(-c4ccccc4)cs3)c2c1] |
| 417 | 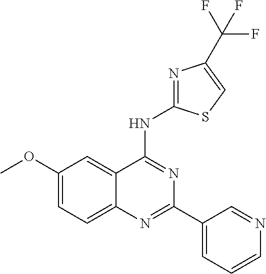 [SMILES: COc1ccc2nc(-c3cccnc3)nc(Nc3nc(C(F)(F)F)cs3)c2c1] |
| 418 | 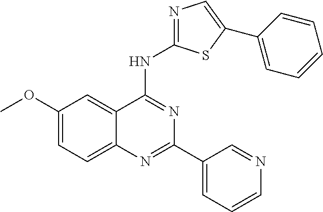 [SMILES: COc1ccc2nc(-c3cccnc3)nc(Nc3ncc(-c4ccccc4)s3)c2c1] |
| 419 | 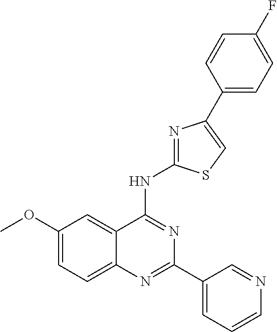 [SMILES: COc1ccc2nc(-c3cccnc3)nc(Nc3nc(-c4ccc(F)cc4)cs3)c2c1] |
| 420 | 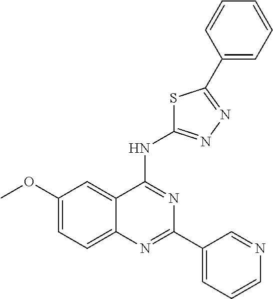 [SMILES: COc1ccc2nc(-c3cccnc3)nc(Nc3nnc(-c4ccccc4)s3)c2c1] |
| 421 | 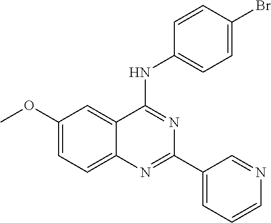 [SMILES: COc1ccc2nc(-c3cccnc3)nc(Nc3ccc(Br)cc3)c2c1] |
| 422 | 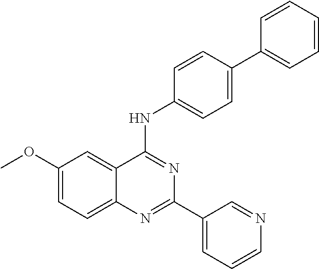 [SMILES: COc1ccc2nc(-c3cccnc3)nc(Nc3ccc(-c4ccccc4)cc3)c2c1] |
| 423 | 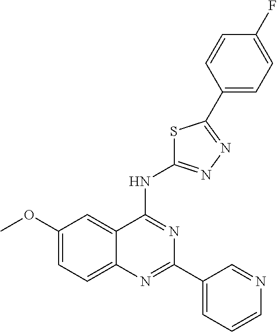 [SMILES: COc1ccc2nc(-c3cccnc3)nc(Nc3nnc(-c4ccc(F)cc4)s3)c2c1] |
| 424 | 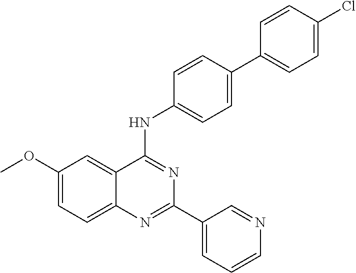 [SMILES: COc1ccc2nc(-c3cccnc3)nc(Nc3ccc(-c4ccc(Cl)cc4)cc3)c2c1] |
| 425 | 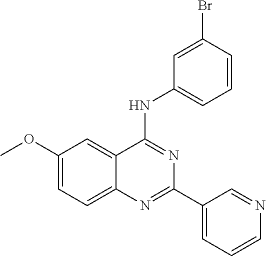 [SMILES: COc1ccc2nc(-c3cccnc3)nc(Nc3cccc(Br)c3)c2c1] |
| 426 | 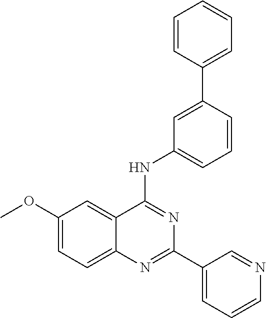 [SMILES: COc1ccc2nc(-c3cccnc3)nc(Nc3cccc(-c4ccccc4)c3)c2c1] |
| 427 | 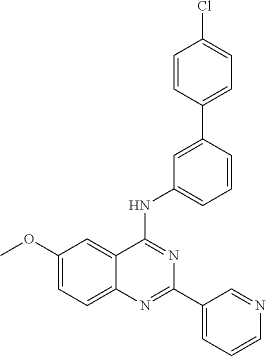 [SMILES: COc1ccc2nc(-c3cccnc3)nc(Nc3cccc(-c4ccc(Cl)cc4)c3)c2c1] |
| 428 | 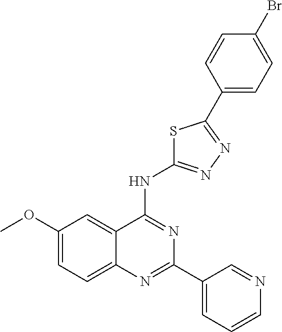 [SMILES: COc1ccc2nc(-c3cccnc3)nc(Nc3nnc(-c4ccc(Br)cc4)s3)c2c1] |
| 429 | 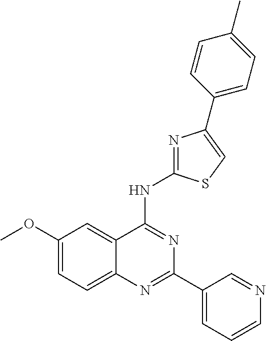 [SMILES: COc1ccc2nc(-c3cccnc3)nc(Nc3nc(-c4ccc(C)cc4)cs3)c2c1] |
| 430 | 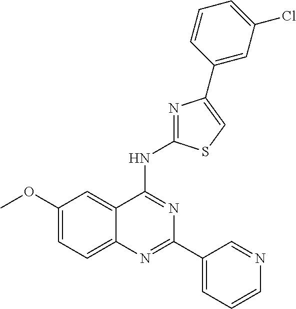 [SMILES: COc1ccc2nc(-c3cccnc3)nc(Nc3nc(-c4cccc(Cl)c4)cs3)c2c1] |
| 431 | 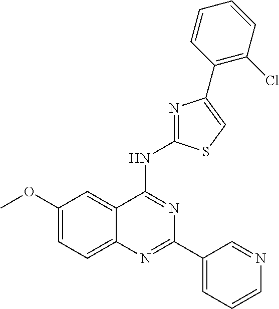 [SMILES: COc1ccc2nc(-c3cccnc3)nc(Nc3nc(-c4ccccc4Cl)cs3)c2c1] |
| 432 | 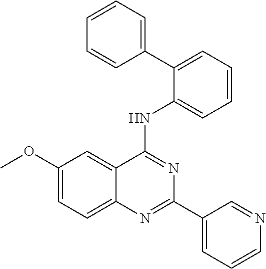 [SMILES: COc1ccc2nc(-c3cccnc3)nc(Nc3ccccc3-c3ccccc3)c2c1] |
| 433 | 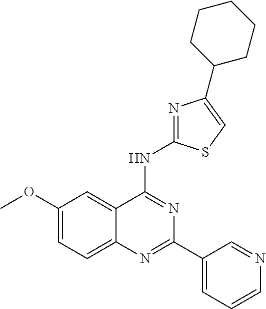 [SMILES: COc1ccc2nc(-c3cccnc3)nc(Nc3nc(C4CCCCC4)cs3)c2c1] |
| 434 | 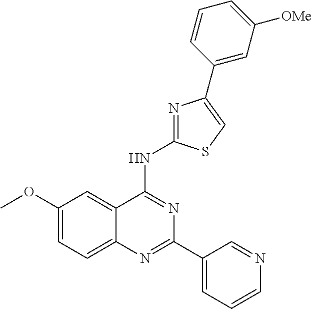 [SMILES: COc1cccc(-c2csc(Nc3nc(-c4cccnc4)nc4ccc(OC)cc34)n2)c1] |
| 435 | 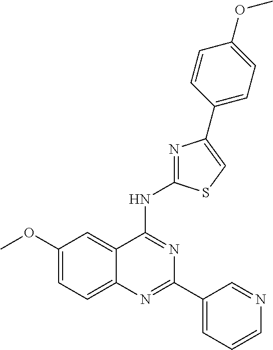 |
| 436 | 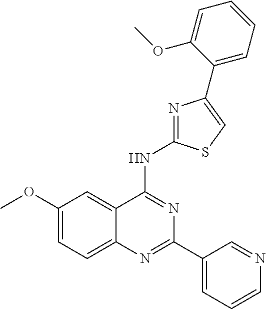 [SMILES: COc1ccc2nc(-c3cccnc3)nc(Nc3nc(-c4ccccc4OC)cs3)c2c1] |
| 437 | 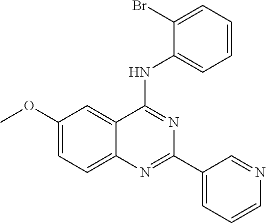 [SMILES: COc1ccc2nc(-c3cccnc3)nc(Nc3ccccc3Br)c2c1] |
| 438 | 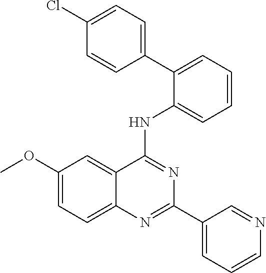 [SMILES: COc1ccc2nc(-c3cccnc3)nc(Nc3ccccc3-c3ccc(Cl)cc3)c2c1] |
| 439 | 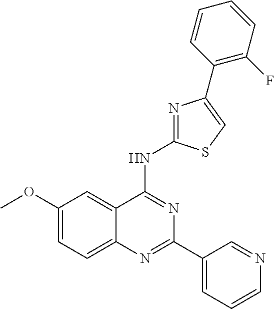 [SMILES: COc1ccc2nc(-c3cccnc3)nc(Nc3nc(-c4ccccc4F)cs3)c2c1] |
| 440 | 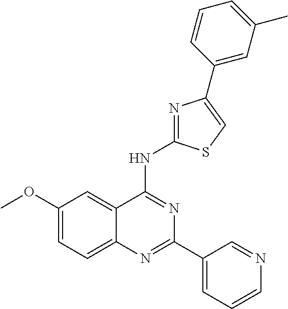 |
| 441 | 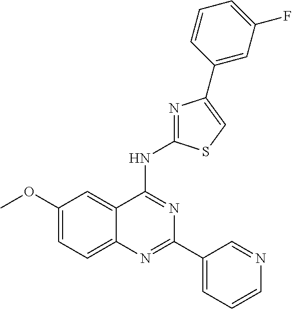 [SMILES: COc1ccc2nc(-c3cccnc3)nc(Nc3nc(-c4cccc(F)c4)cs3)c2c1] |
| 442 | 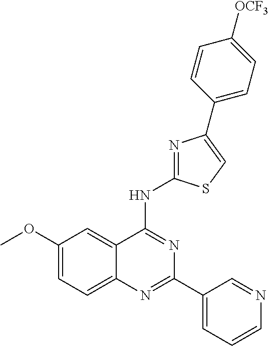 [SMILES: COc1ccc2nc(-c3cccnc3)nc(Nc3nc(-c4ccc(OC(F)(F)F)cc4)cs3)c2c1] |
| 443 | 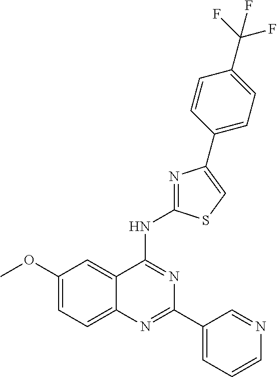 [SMILES: COc1ccc2nc(-c3cccnc3)nc(Nc3nc(-c4ccc(C(F)(F)F)cc4)cs3)c2c1] |
| 444 | 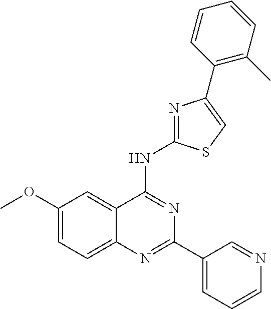 |
| 445 | 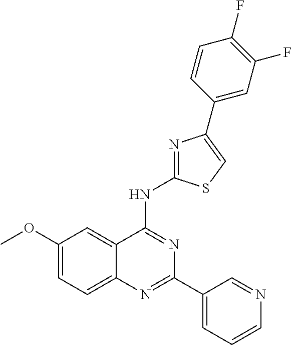 [SMILES: COc1ccc2nc(-c3cccnc3)nc(Nc3nc(-c4ccc(F)c(F)c4)cs3)c2c1] |
| 446 | 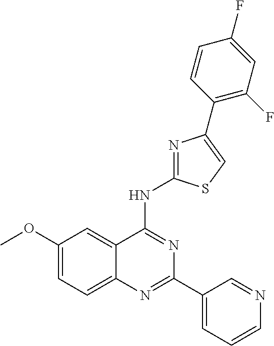 [SMILES: COc1ccc2nc(-c3cccnc3)nc(Nc3nc(-c4ccc(F)cc4F)cs3)c2c1] |
| 447 | 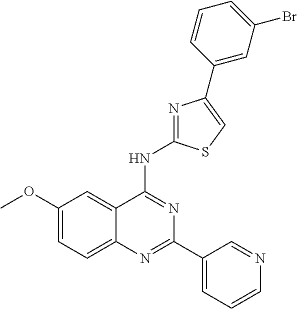 [SMILES: COc1ccc2nc(-c3cccnc3)nc(Nc3nc(-c4cccc(Br)c4)cs3)c2c1] |
| 448 | 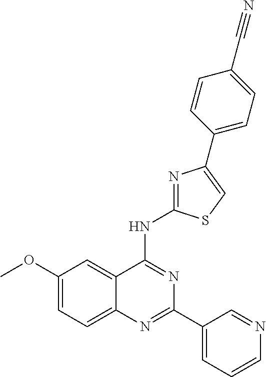 [SMILES: COc1ccc2nc(-c3cccnc3)nc(Nc3nc(-c4ccc(C#N)cc4)cs3)c2c1] |
| 449 | 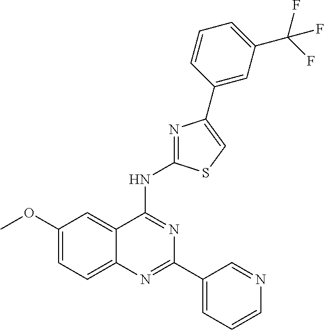 [SMILES: COc1ccc2nc(-c3cccnc3)nc(Nc3nc(-c4cccc(C(F)(F)F)c4)cs3)c2c1] |
| 450 | 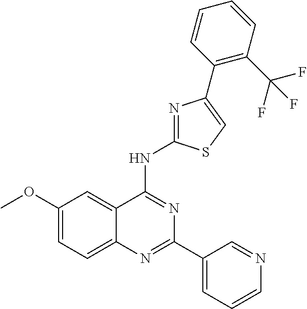 [SMILES: COc1ccc2nc(-c3cccnc3)nc(Nc3nc(-c4ccccc4C(F)(F)F)cs3)c2c1] |
| 451 | 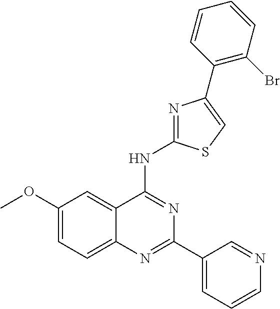 [SMILES: COc1ccc2nc(-c3cccnc3)nc(Nc3nc(-c4ccccc4Br)cs3)c2c1] |
| 452 | 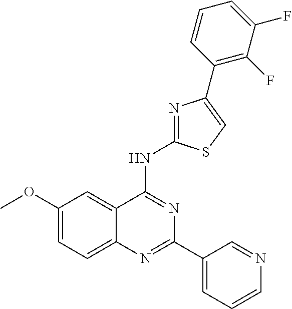 [SMILES: COc1ccc2nc(-c3cccnc3)nc(Nc3nc(-c4cccc(F)c4F)cs3)c2c1] |
| 453 | 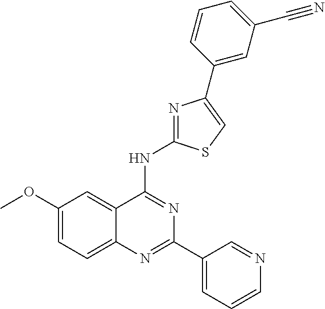 [SMILES: COc1ccc2nc(-c3cccnc3)nc(Nc3nc(-c4cccc(C#N)c4)cs3)c2c1] |
| 454 | 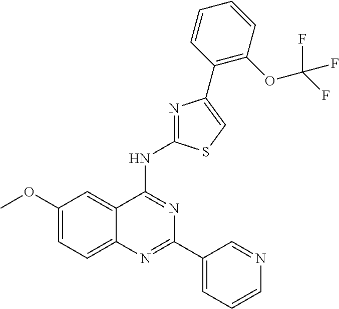 [SMILES: COc1ccc2nc(-c3cccnc3)nc(Nc3nc(-c4ccccc4OC(F)(F)F)cs3)c2c1] |
| 455 | 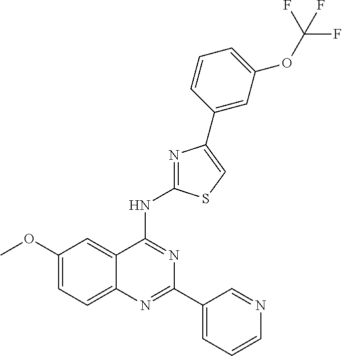 [SMILES: COc1ccc2nc(-c3cccnc3)nc(Nc3nc(-c4cccc(OC(F)(F)F)c4)cs3)c2c1] |
| 456 | 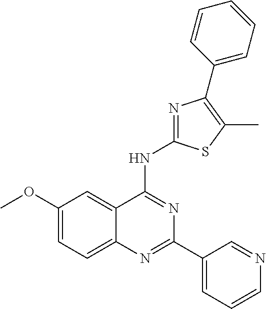 [SMILES: COc1ccc2nc(-c3cccnc3)nc(Nc3nc(-c4ccccc4)c(C)s3)c2c1] |
| 457 | 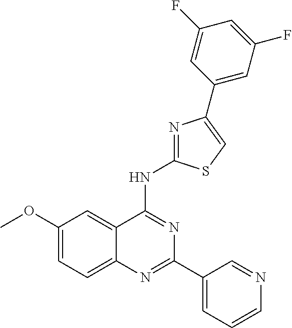 [SMILES: COc1ccc2nc(-c3cccnc3)nc(Nc3nc(-c4cc(F)cc(F)c4)cs3)c2c1] |
| 458 | 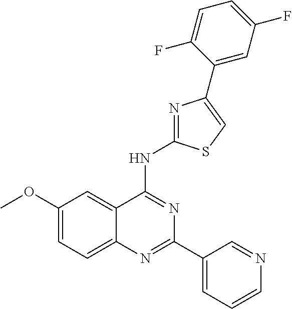 [SMILES: COc1ccc2nc(-c3cccnc3)nc(Nc3nc(-c4cc(F)ccc4F)cs3)c2c1] |
| 459 | 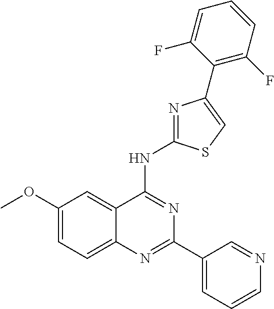 [SMILES: COc1ccc2nc(-c3cccnc3)nc(Nc3nc(-c4c(F)cccc4F)cs3)c2c1] |
| 460 | 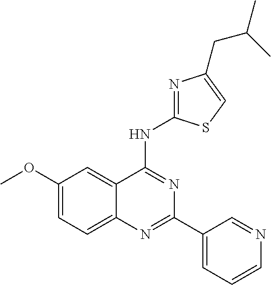 [SMILES: COc1ccc2nc(-c3cccnc3)nc(Nc3nc(CC(C)C)cs3)c2c1] |
| 461 | 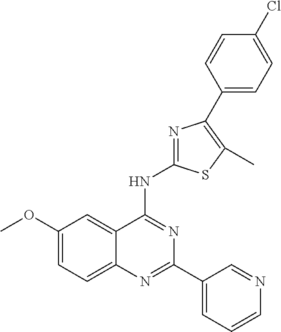 [SMILES: COc1ccc2nc(-c3cccnc3)nc(Nc3nc(-c4ccc(Cl)cc4)c(C)s3)c2c1] |
| 462 | 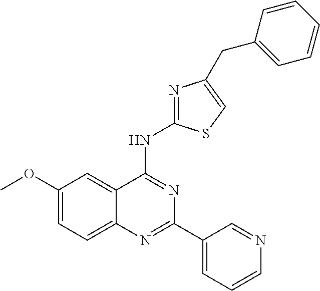 [SMILES: COc1ccc2nc(-c3cccnc3)nc(Nc3nc(Cc4ccccc4)cs3)c2c1] |
| 463 | 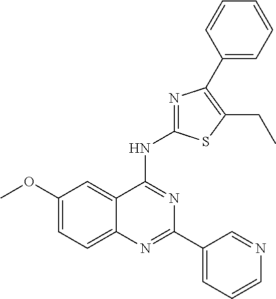 [SMILES: CCc1sc(Nc2nc(-c3cccnc3)nc3ccc(OC)cc23)nc1-c1ccccc1] |
| 464 | 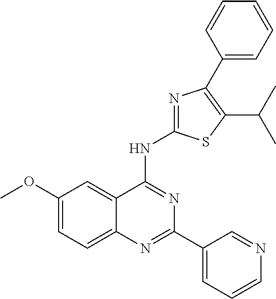 [SMILES: COc1ccc2nc(-c3cccnc3)nc(Nc3nc(-c4ccccc4)c(C(C)C)s3)c2c1] |
| 465 | 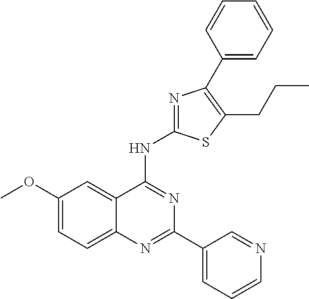 [SMILES: CCCc1sc(Nc2nc(-c3cccnc3)nc3ccc(OC)cc23)nc1-c1ccccc1] |
| 466 | 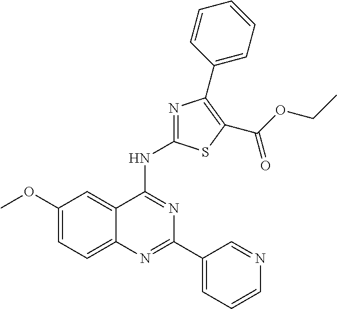 [SMILES: CCOC(=O)c1sc(Nc2nc(-c3cccnc3)nc3ccc(OC)cc23)nc1-c1ccccc1] |
| 467 | 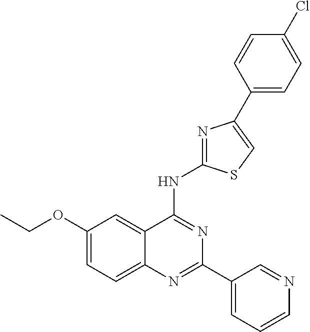 [SMILES: CCOc1ccc2nc(-c3cccnc3)nc(Nc3nc(-c4ccc(Cl)cc4)cs3)c2c1] |
| 468 | 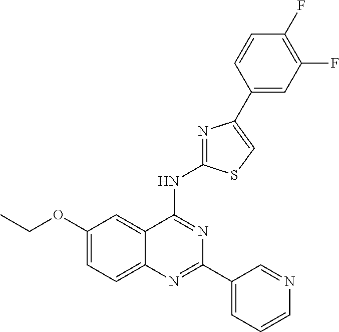 [SMILES: CCOc1ccc2nc(-c3cccnc3)nc(Nc3nc(-c4ccc(F)c(F)c4)cs3)c2c1] |
| 469 | 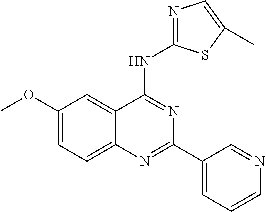 [SMILES: COc1ccc2nc(-c3cccnc3)nc(Nc3ncc(C)s3)c2c1] |
| 470 | 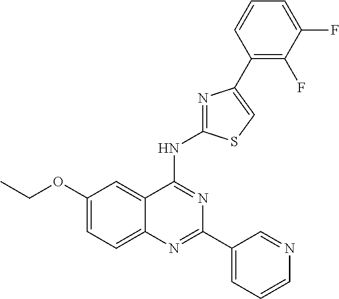 [SMILES: CCOc1ccc2nc(-c3cccnc3)nc(Nc3nc(-c4cccc(F)c4F)cs3)c2c1] |
| 471 | 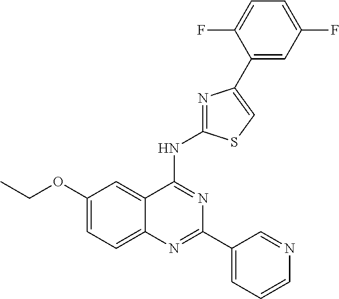 [SMILES: CCOc1ccc2nc(-c3cccnc3)nc(Nc3nc(-c4cc(F)ccc4F)cs3)c2c1] |
| 472 | 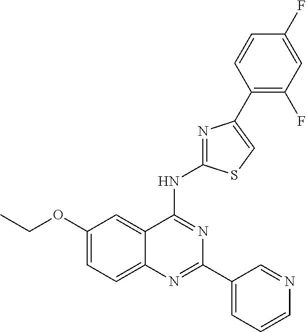 [SMILES: CCOc1ccc2nc(-c3cccnc3)nc(Nc3nc(-c4ccc(F)cc4F)cs3)c2c1] |
| 473 | 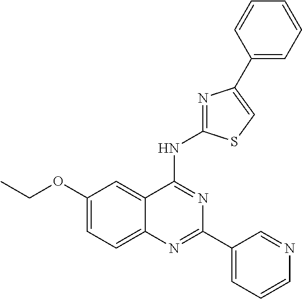 [SMILES: CCOc1ccc2nc(-c3cccnc3)nc(Nc3nc(-c4ccccc4)cs3)c2c1] |
| 474 | 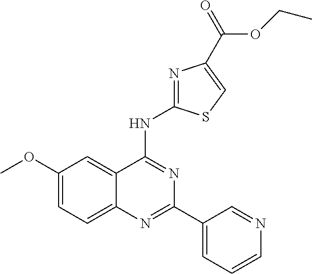 [SMILES: CCOC(=O)c1csc(Nc2nc(-c3cccnc3)nc3ccc(OC)cc23)n1] |
| 475 | 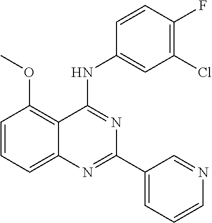 [SMILES: COc1cccc2nc(-c3cccnc3)nc(Nc3ccc(F)c(Cl)c3)c12] |
| 476 | 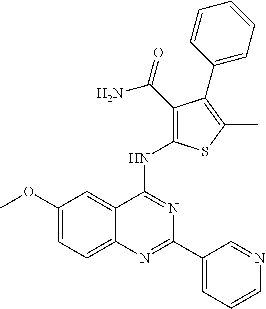 [SMILES: COc1ccc2nc(-c3cccnc3)nc(Nc3sc(C)c(-c4ccccc4)c3C(N)=O)c2c1] |
| 477 | 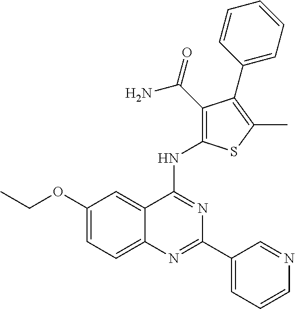 [SMILES: CCOc1ccc2nc(-c3cccnc3)nc(Nc3sc(C)c(-c4ccccc4)c3C(N)=O)c2c1] |
| 478 | 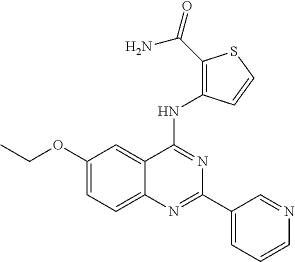 [SMILES: CCOc1ccc2nc(-c3cccnc3)nc(Nc3ccsc3C(N)=O)c2c1] |
| 479 | 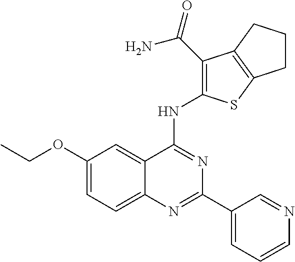 [SMILES: CCOc1ccc2nc(-c3cccnc3)nc(Nc3sc4c(c3C(N)=O)CCC4)c2c1] |
| 480 | 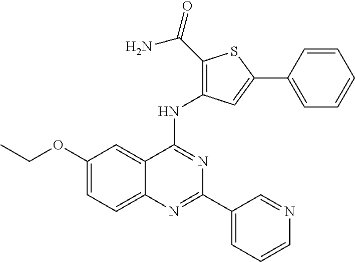 [SMILES: CCOc1ccc2nc(-c3cccnc3)nc(Nc3cc(-c4ccccc4)sc3C(N)=O)c2c1] |
| 481 | 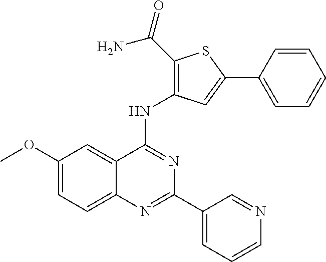 [SMILES: COc1ccc2nc(-c3cccnc3)nc(Nc3cc(-c4ccccc4)sc3C(N)=O)c2c1] |
| 482 | 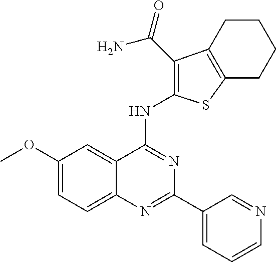 [SMILES: COc1ccc2nc(-c3cccnc3)nc(Nc3sc4c(c3C(N)=O)CCCC4)c2c1] |
| 483 | 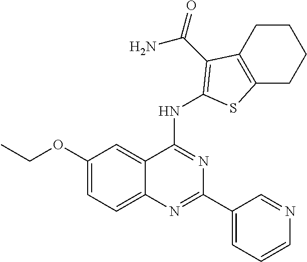 [SMILES: CCOc1ccc2nc(-c3cccnc3)nc(Nc3sc4c(c3C(N)=O)CCCC4)c2c1] |
| 484 | 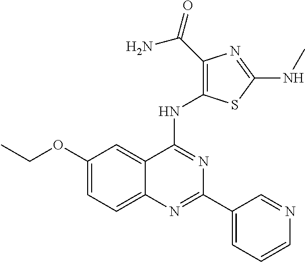 [SMILES: CCOc1ccc2nc(-c3cccnc3)nc(Nc3sc(NC)nc3C(N)=O)c2c1] |
| 485 | 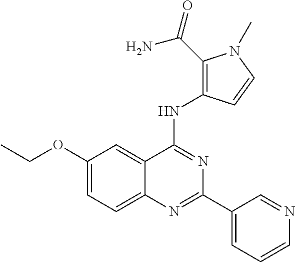 [SMILES: CCOc1ccc2nc(-c3cccnc3)nc(Nc3ccn(C)c3C(N)=O)c2c1] |
| 486 | 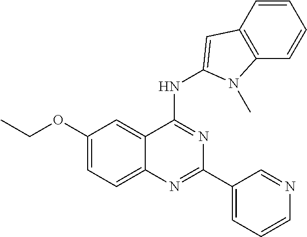 [SMILES: CCOc1ccc2nc(-c3cccnc3)nc(Nc3cc4ccccc4n3C)c2c1] |
| 487 | 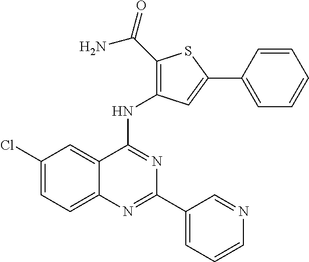 [SMILES: NC(=O)c1sc(-c2ccccc2)cc1Nc1nc(-c2cccnc2)nc2ccc(Cl)cc12] |
| 488 | 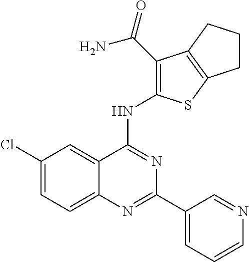 [SMILES: NC(=O)c1c(Nc2nc(-c3cccnc3)nc3ccc(Cl)cc23)sc2c1CCC2] |
| 489 | 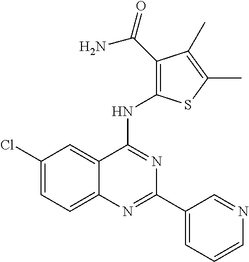 [SMILES: Cc1sc(Nc2nc(-c3cccnc3)nc3ccc(Cl)cc23)c(C(N)=O)c1C] |
| 490 | 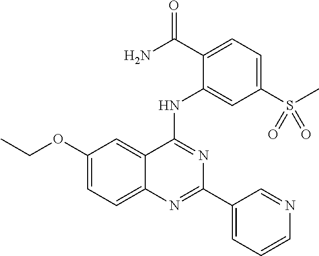 [SMILES: CCOc1ccc2nc(-c3cccnc3)nc(Nc3cc(S(C)(=O)=O)ccc3C(N)=O)c2c1] |
| 491 | 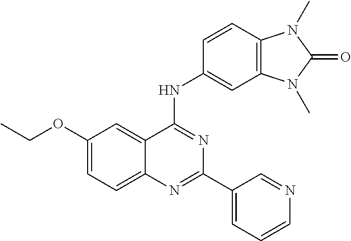 [SMILES: CCOc1ccc2nc(-c3cccnc3)nc(Nc3ccc4c(c3)n(C)c(=O)n4C)c2c1] |
| 492 | 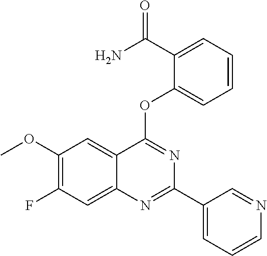 [SMILES: COc1cc2c(Oc3ccccc3C(N)=O)nc(-c3cccnc3)nc2cc1F] |
| 493 | 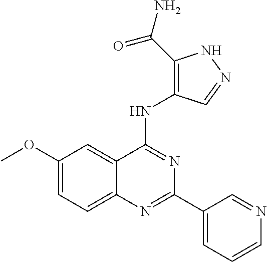 [SMILES: COc1ccc2nc(-c3cccnc3)nc(Nc3cn[nH]c3C(N)=O)c2c1] |
| 494 | 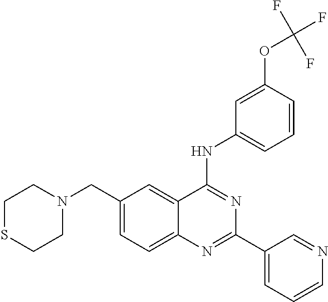 [SMILES: FC(F)(F)Oc1cccc(Nc2nc(-c3cccnc3)nc3ccc(CN4CCSCC4)cc23)c1] |
| 495 | 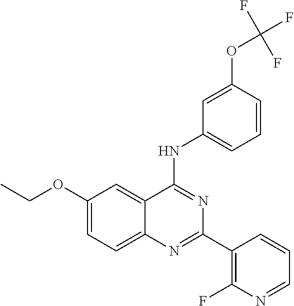 [SMILES: CCOc1ccc2nc(-c3cccnc3F)nc(Nc3cccc(OC(F)(F)F)c3)c2c1] |
| 496 | 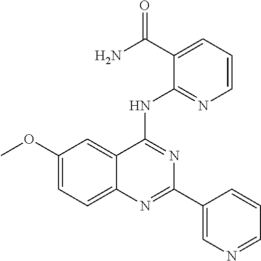 [SMILES: COc1ccc2nc(-c3cccnc3)nc(Nc3ncccc3C(N)=O)c2c1] |
| 497 | 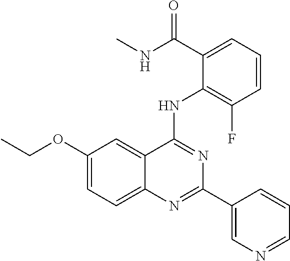 [SMILES: CCOc1ccc2nc(-c3cccnc3)nc(Nc3c(F)cccc3C(=O)NC)c2c1] |
| 498 | 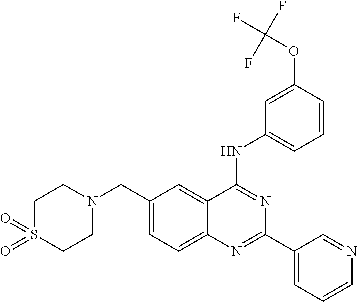 [SMILES: O=S1(=O)CCN(Cc2ccc3nc(-c4cccnc4)nc(Nc4cccc(OC(F)(F)F)c4)c3c2)CC1] |
| 499 | 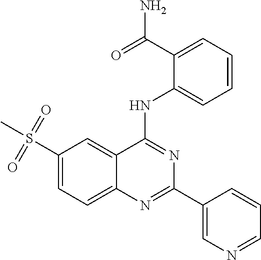 [SMILES: CS(=O)(=O)c1ccc2nc(-c3cccnc3)nc(Nc3ccccc3C(N)=O)c2c1] |
| 501 | 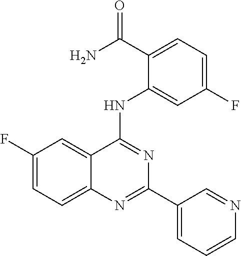 [SMILES: NC(=O)c1ccc(F)cc1Nc1nc(-c2cccnc2)nc2ccc(F)cc12] |
| 502 | 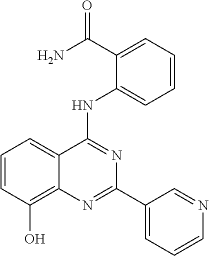 [SMILES: NC(=O)c1ccccc1Nc1nc(-c2cccnc2)nc2c(O)cccc12] |
| 503 | 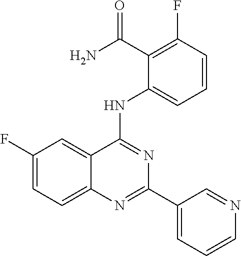 [SMILES: NC(=O)c1c(F)cccc1Nc1nc(-c2cccnc2)nc2ccc(F)cc12] |
| 504 | 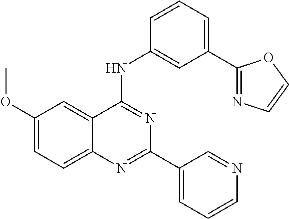 [SMILES: COc1ccc2nc(-c3cccnc3)nc(Nc3cccc(-c4ncco4)c3)c2c1] |
| 505 | 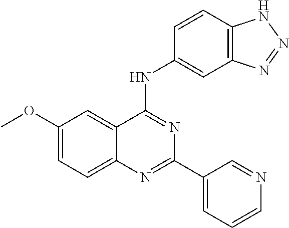 [SMILES: COc1ccc2nc(-c3cccnc3)nc(Nc3ccc4[nH]nnc4c3)c2c1] |
| 506 | 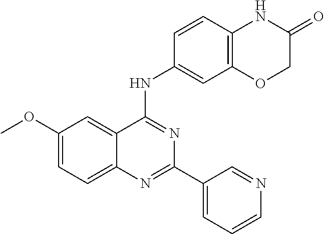 [SMILES: COc1ccc2nc(-c3cccnc3)nc(Nc3ccc4c(c3)OCC(=O)N4)c2c1] |
| 507 | 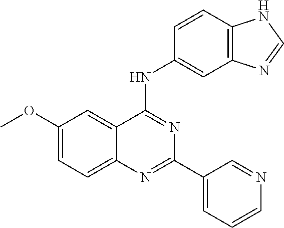 [SMILES: COc1ccc2nc(-c3cccnc3)nc(Nc3ccc4[nH]cnc4c3)c2c1] |
| 508 | 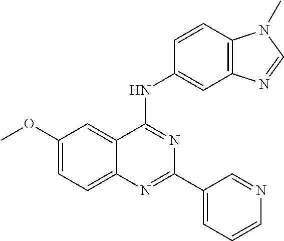 [SMILES: COc1ccc2nc(-c3cccnc3)nc(Nc3ccc4c(c3)ncn4C)c2c1] |
| 509 | 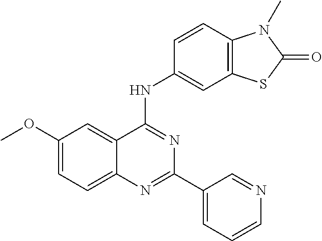 [SMILES: COc1ccc2nc(-c3cccnc3)nc(Nc3ccc4c(c3)sc(=O)n4C)c2c1] |
| 510 | 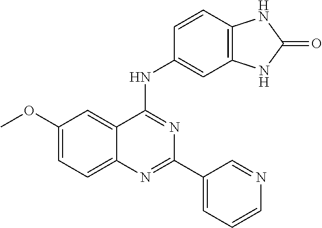 [SMILES: COc1ccc2nc(-c3cccnc3)nc(Nc3ccc4[nH]c(=O)[nH]c4c3)c2c1] |
| 511 | 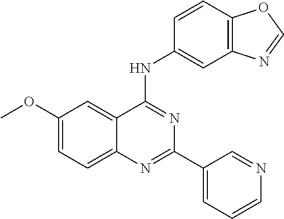 [SMILES: COc1ccc2nc(-c3cccnc3)nc(Nc3ccc4ocnc4c3)c2c1] |
| 512 | 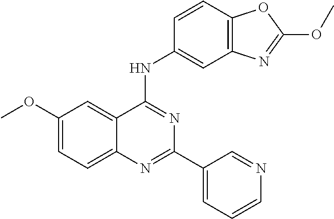 |
| 513 | 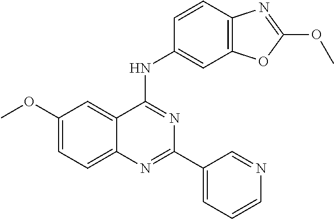 |
| 514 | 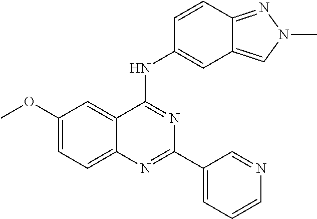 |
| 515 | 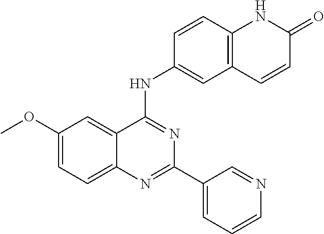 [SMILES: COc1ccc2nc(-c3cccnc3)nc(Nc3ccc4[nH]c(=O)ccc4c3)c2c1] |
| 516 | 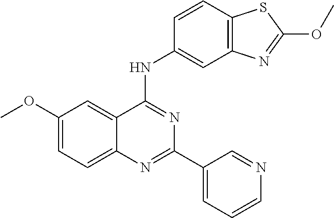 |
| 517 | 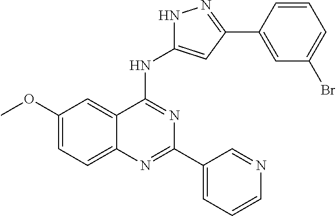 [SMILES: COc1ccc2nc(-c3cccnc3)nc(Nc3cc(-c4cccc(Br)c4)n[nH]3)c2c1] |
| 518 | 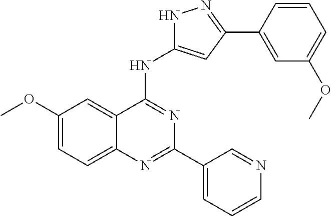 [SMILES: COc1cccc(-c2cc(Nc3nc(-c4cccnc4)nc4ccc(OC)cc34)[nH]n2)c1] |
| 519 | 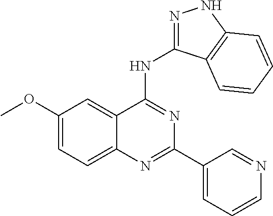 [SMILES: COc1ccc2nc(-c3cccnc3)nc(Nc3n[nH]c4ccccc34)c2c1] |
| 520 | 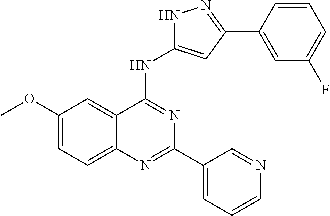 [SMILES: COc1ccc2nc(-c3cccnc3)nc(Nc3cc(-c4cccc(F)c4)n[nH]3)c2c1] |
| 521 | 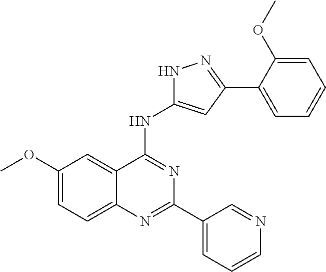 [SMILES: COc1ccc2nc(-c3cccnc3)nc(Nc3cc(-c4ccccc4OC)n[nH]3)c2c1] |
| 1758 | 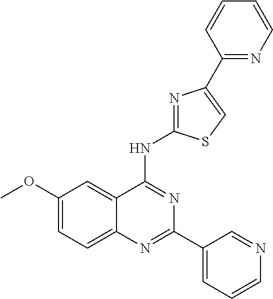 [SMILES: COc1ccc2nc(-c3cccnc3)nc(Nc3nc(-c4ccccn4)cs3)c2c1] |
| 1759 | 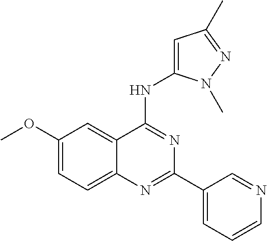 [SMILES: COc1ccc2nc(-c3cccnc3)nc(Nc3cc(C)nn3C)c2c1] |
| 522 | 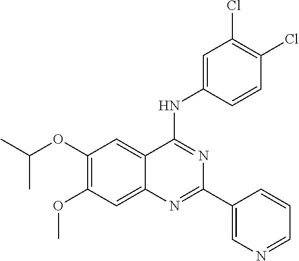 [SMILES: COc1cc2nc(-c3cccnc3)nc(Nc3ccc(Cl)c(Cl)c3)c2cc1OC(C)C] |
| 523 | 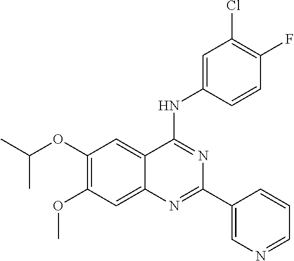 [SMILES: COc1cc2nc(-c3cccnc3)nc(Nc3ccc(F)c(Cl)c3)c2cc1OC(C)C] |
| 524 | 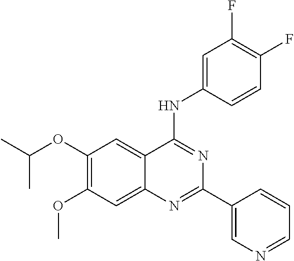 [SMILES: COc1cc2nc(-c3cccnc3)nc(Nc3ccc(F)c(F)c3)c2cc1OC(C)C] |
| 525 | 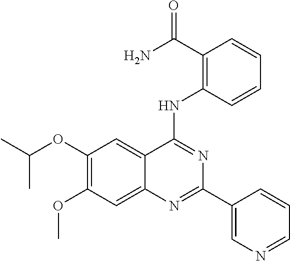 [SMILES: COc1cc2nc(-c3cccnc3)nc(Nc3ccccc3C(N)=O)c2cc1OC(C)C] |
| 526 | 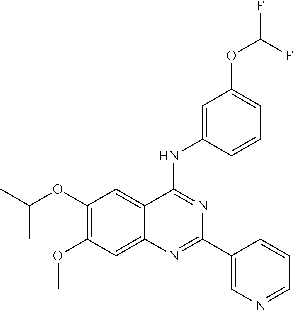 [SMILES: COc1cc2nc(-c3cccnc3)nc(Nc3cccc(OC(F)F)c3)c2cc1OC(C)C] |
| 527 | 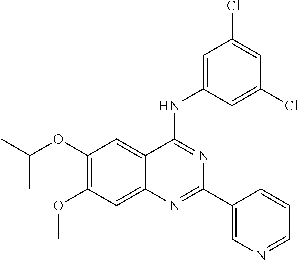 [SMILES: COc1cc2nc(-c3cccnc3)nc(Nc3cc(Cl)cc(Cl)c3)c2cc1OC(C)C] |
| 528 | 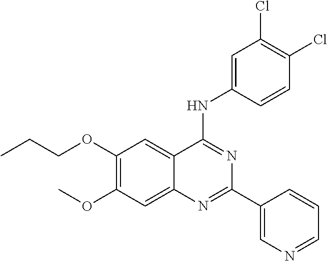 [SMILES: CCCOc1cc2c(Nc3ccc(Cl)c(Cl)c3)nc(-c3cccnc3)nc2cc1OC] |
| 529 | 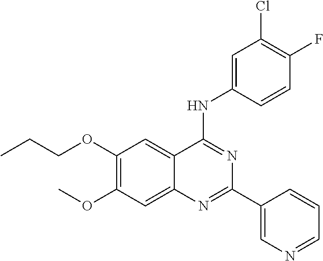 [SMILES: CCCOc1cc2c(Nc3ccc(F)c(Cl)c3)nc(-c3cccnc3)nc2cc1OC] |
| 530 | 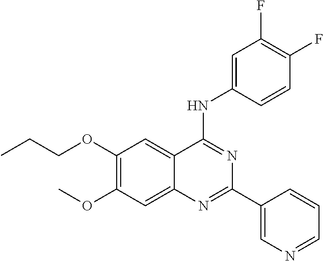 [SMILES: CCCOc1cc2c(Nc3ccc(F)c(F)c3)nc(-c3cccnc3)nc2cc1OC] |
| 531 | 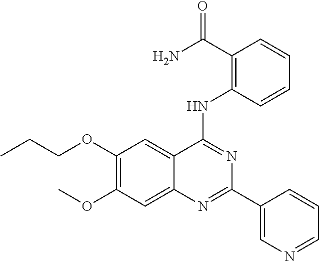 [SMILES: CCCOc1cc2c(Nc3ccccc3C(N)=O)nc(-c3cccnc3)nc2cc1OC] |
| 532 | 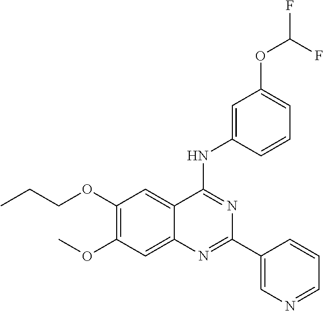 [SMILES: CCCOc1cc2c(Nc3cccc(OC(F)F)c3)nc(-c3cccnc3)nc2cc1OC] |
| 533 | 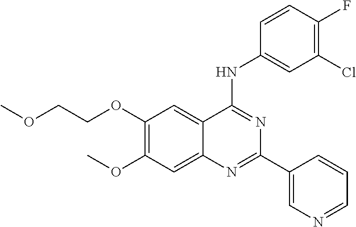 [SMILES: COCCOc1cc2c(Nc3ccc(F)c(Cl)c3)nc(-c3cccnc3)nc2cc1OC] |
| 534 | 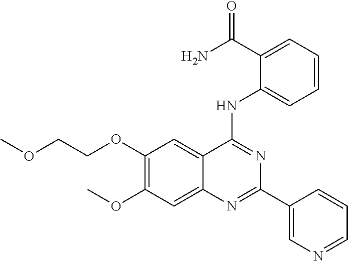 [SMILES: COCCOc1cc2c(Nc3ccccc3C(N)=O)nc(-c3cccnc3)nc2cc1OC] |
| 535 | 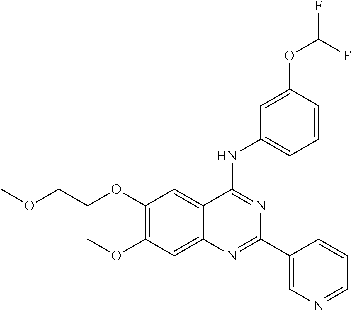 [SMILES: COCCOc1cc2c(Nc3cccc(OC(F)F)c3)nc(-c3cccnc3)nc2cc1OC] |
| 536 | 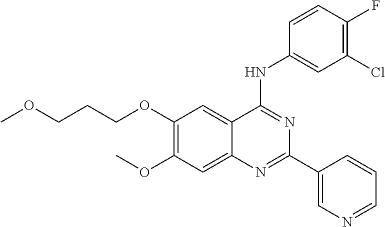 [SMILES: COCCCOc1cc2c(Nc3ccc(F)c(Cl)c3)nc(-c3cccnc3)nc2cc1OC] |
| 537 | 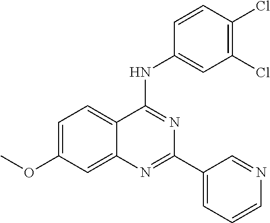 [SMILES: COc1ccc2c(Nc3ccc(Cl)c(Cl)c3)nc(-c3cccnc3)nc2c1] |
| 538 | 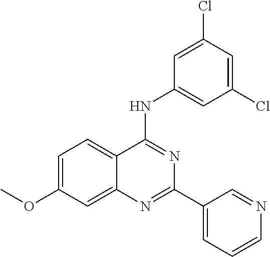 [SMILES: COc1ccc2c(Nc3cc(Cl)cc(Cl)c3)nc(-c3cccnc3)nc2c1] |
| 539 | 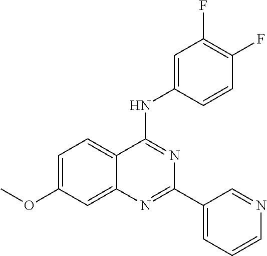 [SMILES: COc1ccc2c(Nc3ccc(F)c(F)c3)nc(-c3cccnc3)nc2c1] |
| 540 | 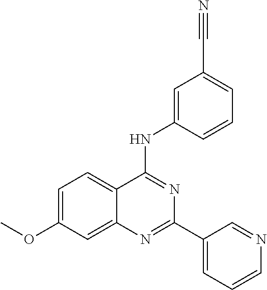 [SMILES: COc1ccc2c(Nc3cccc(C#N)c3)nc(-c3cccnc3)nc2c1] |
| 541 | 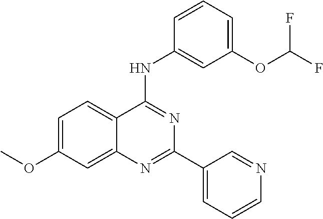 [SMILES: COc1ccc2c(Nc3cccc(OC(F)F)c3)nc(-c3cccnc3)nc2c1] |
| 542 | 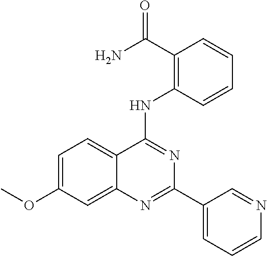 [SMILES: COc1ccc2c(Nc3ccccc3C(N)=O)nc(-c3cccnc3)nc2c1] |
| 543 | 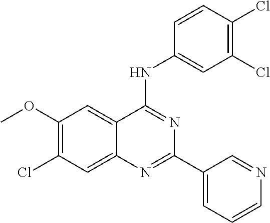 [SMILES: COc1cc2c(Nc3ccc(Cl)c(Cl)c3)nc(-c3cccnc3)nc2cc1Cl] |
| 544 | 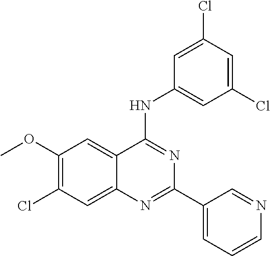 [SMILES: COc1cc2c(Nc3cc(Cl)cc(Cl)c3)nc(-c3cccnc3)nc2cc1Cl] |
| 545 | 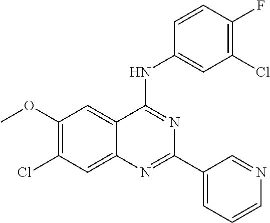 [SMILES: COc1cc2c(Nc3ccc(F)c(Cl)c3)nc(-c3cccnc3)nc2cc1Cl] |
| 546 | 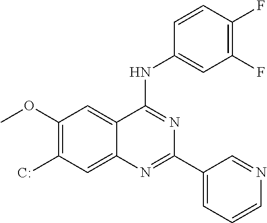 |
| 547 | 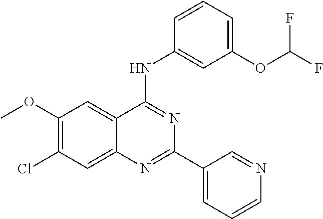 [SMILES: COc1cc2c(Nc3cccc(OC(F)F)c3)nc(-c3cccnc3)nc2cc1Cl] |
| 548 | 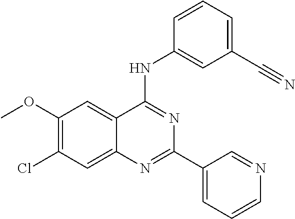 [SMILES: COc1cc2c(Nc3cccc(C#N)c3)nc(-c3cccnc3)nc2cc1Cl] |
| 549 | 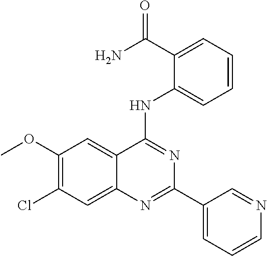 [SMILES: COc1cc2c(Nc3ccccc3C(N)=O)nc(-c3cccnc3)nc2cc1Cl] |
| 550 | 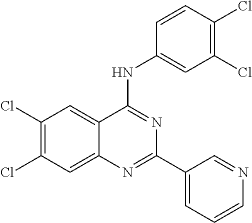 [SMILES: Clc1ccc(Nc2nc(-c3cccnc3)nc3cc(Cl)c(Cl)cc23)cc1Cl] |
| 551 | 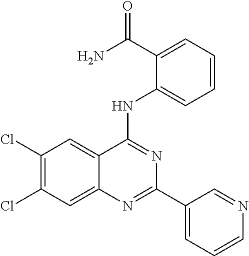 [SMILES: NC(=O)c1ccccc1Nc1nc(-c2cccnc2)nc2cc(Cl)c(Cl)cc12] |
| 552 | 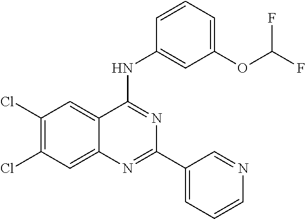 [SMILES: FC(F)Oc1cccc(Nc2nc(-c3cccnc3)nc3cc(Cl)c(Cl)cc23)c1] |
| 553 | 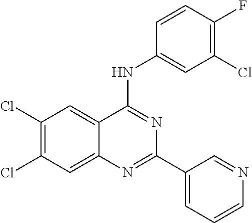 [SMILES: Fc1ccc(Nc2nc(-c3cccnc3)nc3cc(Cl)c(Cl)cc23)cc1Cl] |
| 554 | 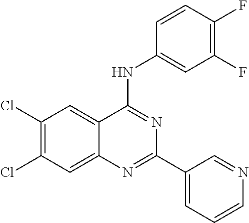 [SMILES: Fc1ccc(Nc2nc(-c3cccnc3)nc3cc(Cl)c(Cl)cc23)cc1F] |
| 555 | 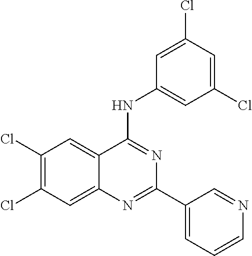 [SMILES: Clc1cc(Cl)cc(Nc2nc(-c3cccnc3)nc3cc(Cl)c(Cl)cc23)c1] |
| 556 | 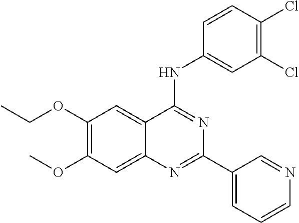 [SMILES: CCOc1cc2c(Nc3ccc(Cl)c(Cl)c3)nc(-c3cccnc3)nc2cc1OC] |
| 557 | 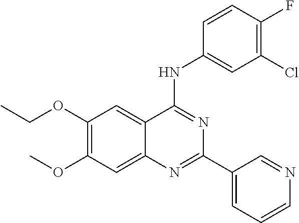 [SMILES: CCOc1cc2c(Nc3ccc(F)c(Cl)c3)nc(-c3cccnc3)nc2cc1OC] |
| 558 | 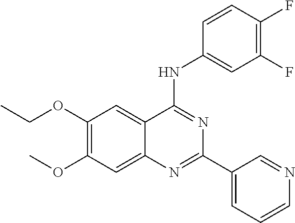 [SMILES: CCOc1cc2c(Nc3ccc(F)c(F)c3)nc(-c3cccnc3)nc2cc1OC] |
| 559 | 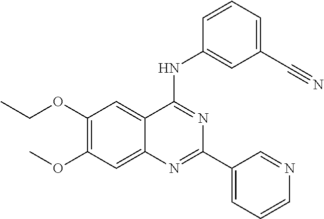 [SMILES: CCOc1cc2c(Nc3cccc(C#N)c3)nc(-c3cccnc3)nc2cc1OC] |
| 560 | 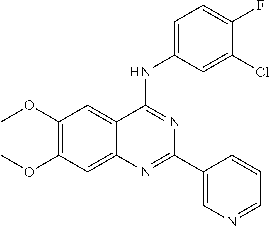 [SMILES: COc1cc2nc(-c3cccnc3)nc(Nc3ccc(F)c(Cl)c3)c2cc1OC] |
| 561 | 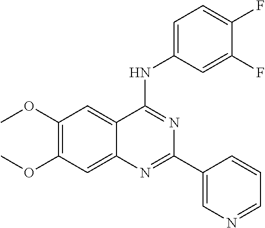 [SMILES: COc1cc2nc(-c3cccnc3)nc(Nc3ccc(F)c(F)c3)c2cc1OC] |
| 562 | 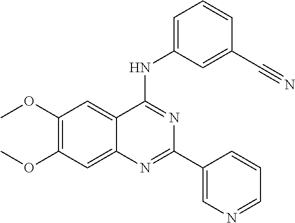 [SMILES: COc1cc2nc(-c3cccnc3)nc(Nc3cccc(C#N)c3)c2cc1OC] |
| 563 | 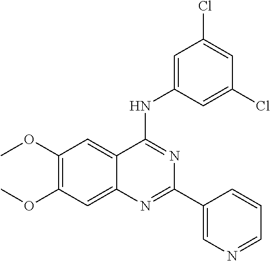 [SMILES: COc1cc2nc(-c3cccnc3)nc(Nc3cc(Cl)cc(Cl)c3)c2cc1OC] |
| 564 | 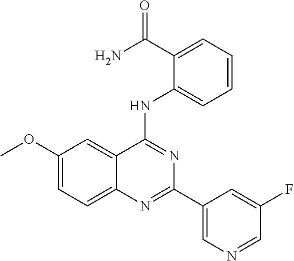 [SMILES: COc1ccc2nc(-c3cncc(F)c3)nc(Nc3ccccc3C(N)=O)c2c1] |
| 565 | 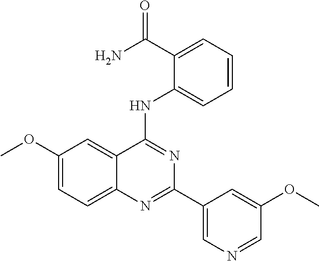 |
| 566 | 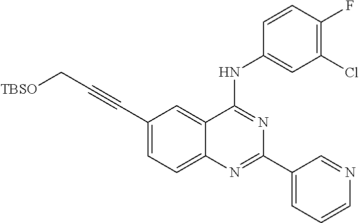 [SMILES: CC(C)(C)[Si](C)(C)OCC#Cc1ccc2nc(-c3cccnc3)nc(Nc3ccc(F)c(Cl)c3)c2c1] |
| 567 | 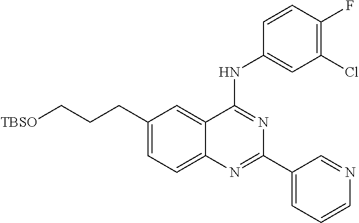 [SMILES: CC(C)(C)[Si](C)(C)OCCCc1ccc2nc(-c3cccnc3)nc(Nc3ccc(F)c(Cl)c3)c2c1] |
| 568 | 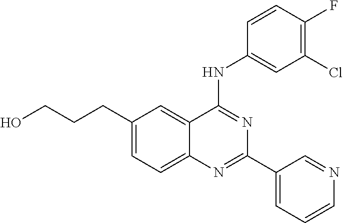 [SMILES: OCCCc1ccc2nc(-c3cccnc3)nc(Nc3ccc(F)c(Cl)c3)c2c1] |
| 569 | 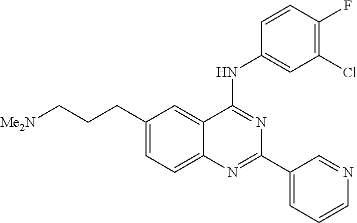 [SMILES: CN(C)CCCc1ccc2nc(-c3cccnc3)nc(Nc3ccc(F)c(Cl)c3)c2c1] |
| 570 | 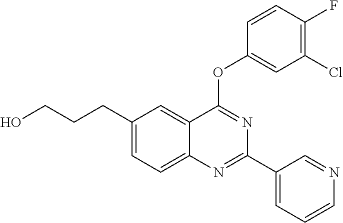 [SMILES: OCCCc1ccc2nc(-c3cccnc3)nc(Oc3ccc(F)c(Cl)c3)c2c1] |
| 571 | 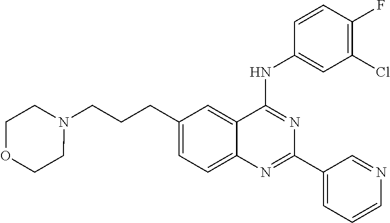 [SMILES: Fc1ccc(Nc2nc(-c3cccnc3)nc3ccc(CCCN4CCOCC4)cc23)cc1Cl] |
| 572 | 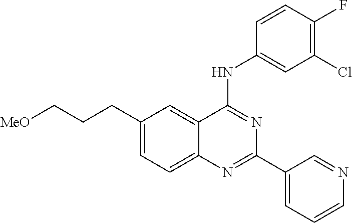 [SMILES: COCCCc1ccc2nc(-c3cccnc3)nc(Nc3ccc(F)c(Cl)c3)c2c1] |
| 573 | 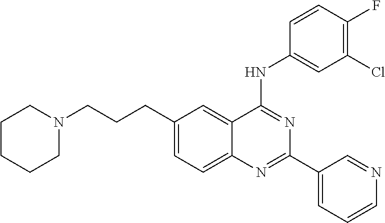 [SMILES: Fc1ccc(Nc2nc(-c3cccnc3)nc3ccc(CCCN4CCCCC4)cc23)cc1Cl] |
| 574 | 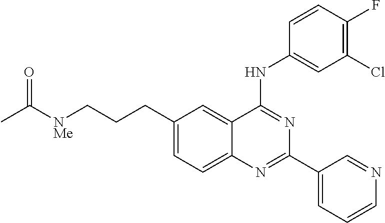 [SMILES: CC(=O)N(C)CCCc1ccc2nc(-c3cccnc3)nc(Nc3ccc(F)c(Cl)c3)c2c1] |
| 575 | 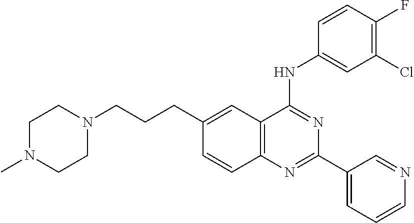 [SMILES: CN1CCN(CCCc2ccc3nc(-c4cccnc4)nc(Nc4ccc(F)c(Cl)c4)c3c2)CC1] |
| 576 | 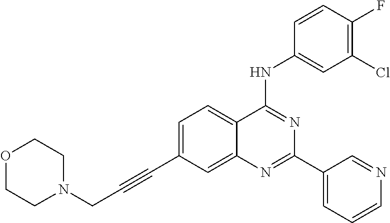 [SMILES: Fc1ccc(Nc2nc(-c3cccnc3)nc3cc(C#CCN4CCOCC4)ccc23)cc1Cl] |
| 577 | 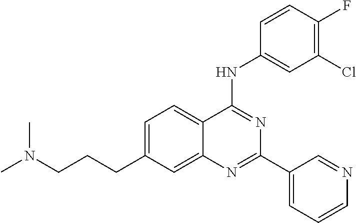 [SMILES: CN(C)CCCc1ccc2c(Nc3ccc(F)c(Cl)c3)nc(-c3cccnc3)nc2c1] |
| 578 | 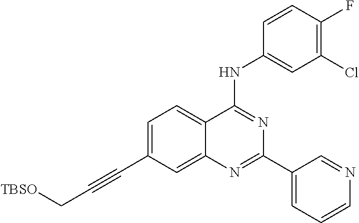 [SMILES: CC(C)(C)[Si](C)(C)OCC#Cc1ccc2c(Nc3ccc(F)c(Cl)c3)nc(-c3cccnc3)nc2c1] |
| 579 | 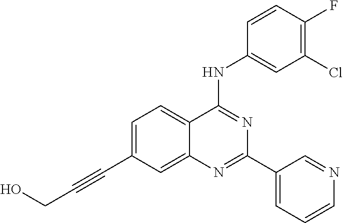 [SMILES: OCC#Cc1ccc2c(Nc3ccc(F)c(Cl)c3)nc(-c3cccnc3)nc2c1] |
| 580 | 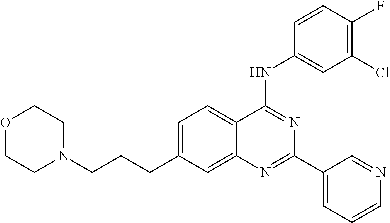 [SMILES: Fc1ccc(Nc2nc(-c3cccnc3)nc3cc(CCCN4CCOCC4)ccc23)cc1Cl] |
| 581 | 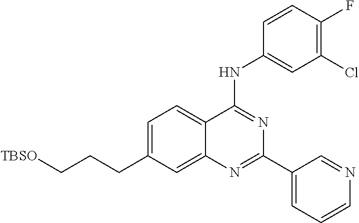 [SMILES: CC(C)(C)[Si](C)(C)OCCCc1ccc2c(Nc3ccc(F)c(Cl)c3)nc(-c3cccnc3)nc2c1] |
| 582 | 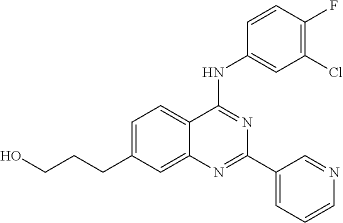 [SMILES: OCCCc1ccc2c(Nc3ccc(F)c(Cl)c3)nc(-c3cccnc3)nc2c1] |
| 583 | 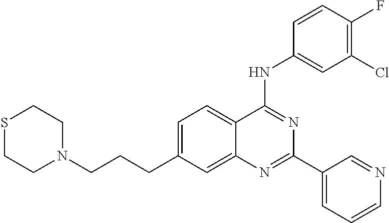 [SMILES: Fc1ccc(Nc2nc(-c3cccnc3)nc3cc(CCCN4CCSCC4)ccc23)cc1Cl] |
| 584 | 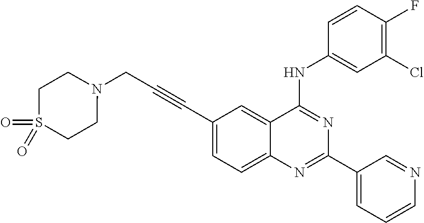 [SMILES: O=S1(=O)CCN(CC#Cc2ccc3nc(-c4cccnc4)nc(Nc4ccc(F)c(Cl)c4)c3c2)CC1] |
| 585 | 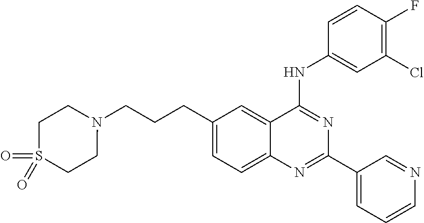 [SMILES: O=S1(=O)CCN(CCCc2ccc3nc(-c4cccnc4)nc(Nc4ccc(F)c(Cl)c4)c3c2)CC1] |
| 586 | 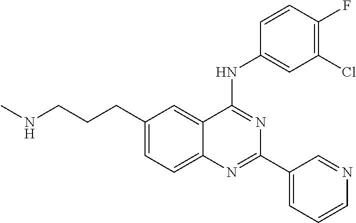 [SMILES: CNCCCc1ccc2nc(-c3cccnc3)nc(Nc3ccc(F)c(Cl)c3)c2c1] |
| 587 | 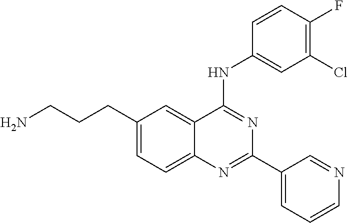 [SMILES: NCCCc1ccc2nc(-c3cccnc3)nc(Nc3ccc(F)c(Cl)c3)c2c1] |
| 588 | 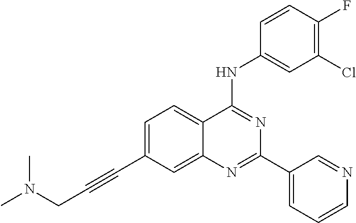 [SMILES: CN(C)CC#Cc1ccc2c(Nc3ccc(F)c(Cl)c3)nc(-c3cccnc3)nc2c1] |
| 589 | 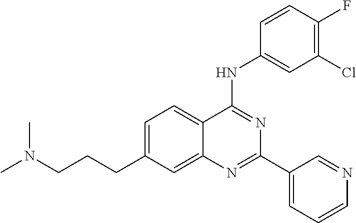 [SMILES: CN(C)CCCc1ccc2c(Nc3ccc(F)c(Cl)c3)nc(-c3cccnc3)nc2c1] |
| 590 | 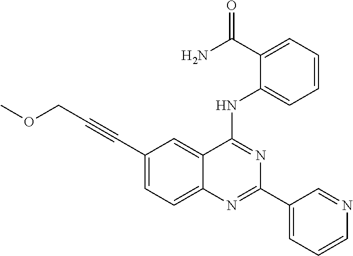 [SMILES: COCC#Cc1ccc2nc(-c3cccnc3)nc(Nc3ccccc3C(N)=O)c2c1] |
| 591 | 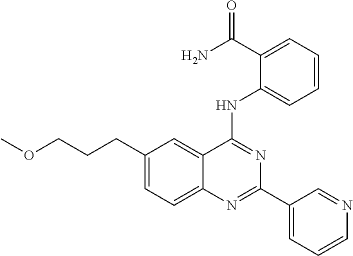 [SMILES: COCCCc1ccc2nc(-c3cccnc3)nc(Nc3ccccc3C(N)=O)c2c1] |
| 592 | 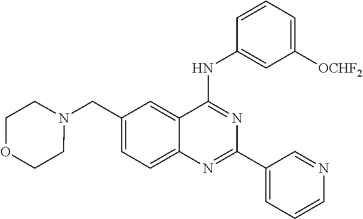 [SMILES: FC(F)Oc1cccc(Nc2nc(-c3cccnc3)nc3ccc(CN4CCOCC4)cc23)c1] |
| 593 | 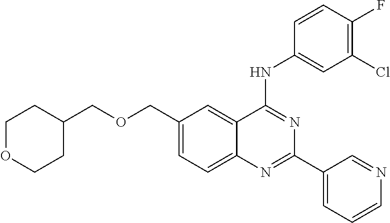 [SMILES: Fc1ccc(Nc2nc(-c3cccnc3)nc3ccc(COCC4CCOCC4)cc23)cc1Cl] |
| 594 | 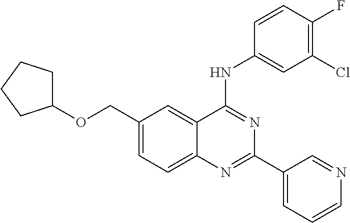 [SMILES: Fc1ccc(Nc2nc(-c3cccnc3)nc3ccc(COC4CCCC4)cc23)cc1Cl] |
| 595 | 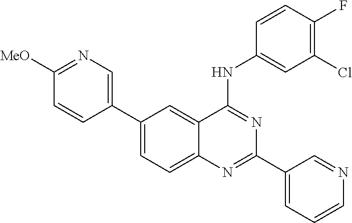 [SMILES: COc1ccc(-c2ccc3nc(-c4cccnc4)nc(Nc4ccc(F)c(Cl)c4)c3c2)cn1] |
| 596 | 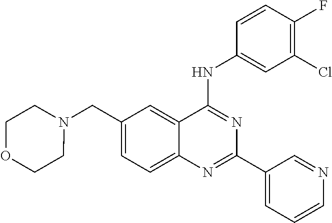 [SMILES: Fc1ccc(Nc2nc(-c3cccnc3)nc3ccc(CN4CCOCC4)cc23)cc1Cl] |
| 597 | 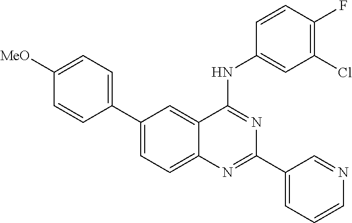 [SMILES: COc1ccc(-c2ccc3nc(-c4cccnc4)nc(Nc4ccc(F)c(Cl)c4)c3c2)cc1] |
| 598 | 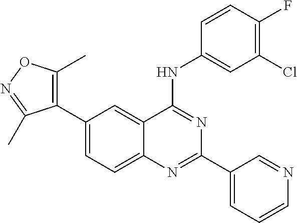 [SMILES: Cc1noc(C)c1-c1ccc2nc(-c3cccnc3)nc(Nc3ccc(F)c(Cl)c3)c2c1] |
| 599 | 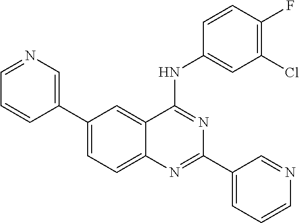 [SMILES: Fc1ccc(Nc2nc(-c3cccnc3)nc3ccc(-c4cccnc4)cc23)cc1Cl] |
| 600 | 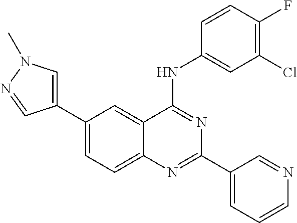 [SMILES: Cn1cc(-c2ccc3nc(-c4cccnc4)nc(Nc4ccc(F)c(Cl)c4)c3c2)cn1] |
| 601 | 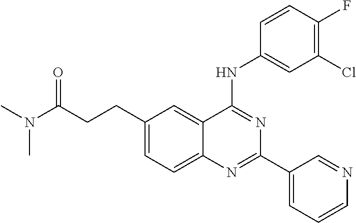 [SMILES: CN(C)C(=O)CCc1ccc2nc(-c3cccnc3)nc(Nc3ccc(F)c(Cl)c3)c2c1] |
| 602 | 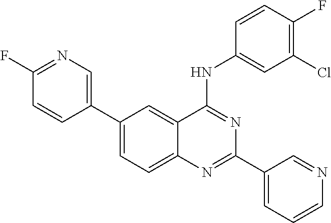 [SMILES: Fc1ccc(-c2ccc3nc(-c4cccnc4)nc(Nc4ccc(F)c(Cl)c4)c3c2)cn1] |
| 603 | 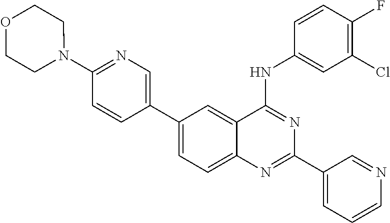 [SMILES: Fc1ccc(Nc2nc(-c3cccnc3)nc3ccc(-c4ccc(N5CCOCC5)nc4)cc23)cc1Cl] |
| 604 | 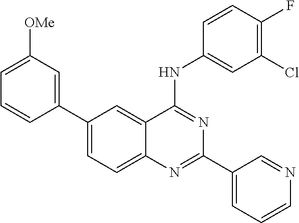 [SMILES: COc1cccc(-c2ccc3nc(-c4cccnc4)nc(Nc4ccc(F)c(Cl)c4)c3c2)c1] |
| 605 | 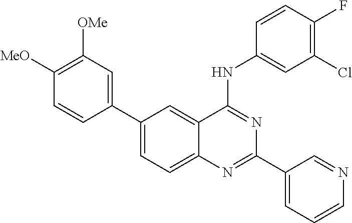 [SMILES: COc1ccc(-c2ccc3nc(-c4cccnc4)nc(Nc4ccc(F)c(Cl)c4)c3c2)cc1OC] |
| 606 | 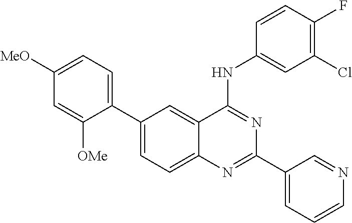 [SMILES: COc1ccc(-c2ccc3nc(-c4cccnc4)nc(Nc4ccc(F)c(Cl)c4)c3c2)c(OC)c1] |
| 607 | 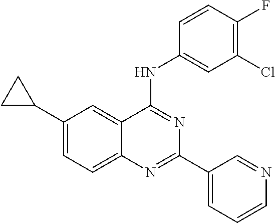 [SMILES: Fc1ccc(Nc2nc(-c3cccnc3)nc3ccc(C4CC4)cc23)cc1Cl] |
| 608 | 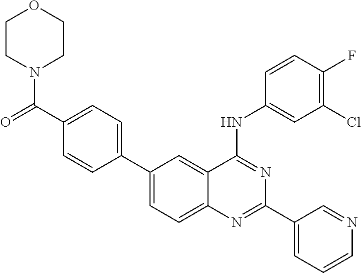 [SMILES: O=C(c1ccc(-c2ccc3nc(-c4cccnc4)nc(Nc4ccc(F)c(Cl)c4)c3c2)cc1)N1CCOCC1] |
| 609 | 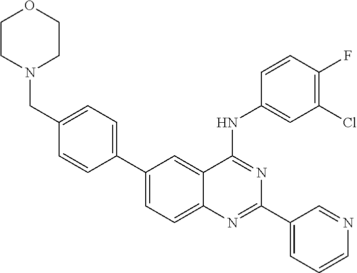 [SMILES: Fc1ccc(Nc2nc(-c3cccnc3)nc3ccc(-c4ccc(CN5CCOCC5)cc4)cc23)cc1Cl] |
| 610 | 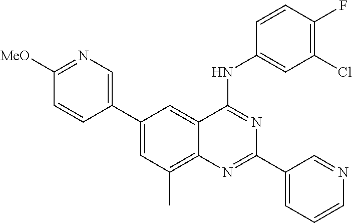 [SMILES: COc1ccc(-c2cc(C)c3nc(-c4cccnc4)nc(Nc4ccc(F)c(Cl)c4)c3c2)cn1] |
| 611 | 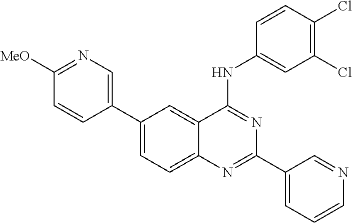 [SMILES: COc1ccc(-c2ccc3nc(-c4cccnc4)nc(Nc4ccc(Cl)c(Cl)c4)c3c2)cn1] |
| 612 | 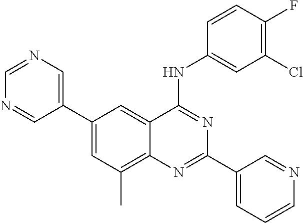 [SMILES: Cc1cc(-c2cncnc2)cc2c(Nc3ccc(F)c(Cl)c3)nc(-c3cccnc3)nc12] |
| 613 | 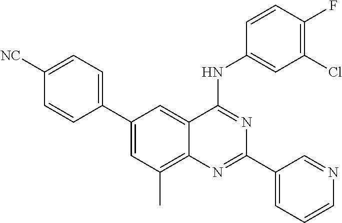 [SMILES: Cc1cc(-c2ccc(C#N)cc2)cc2c(Nc3ccc(F)c(Cl)c3)nc(-c3cccnc3)nc12] |
| 614 | 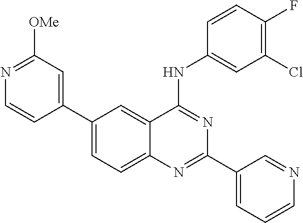 [SMILES: COc1cc(-c2ccc3nc(-c4cccnc4)nc(Nc4ccc(F)c(Cl)c4)c3c2)ccn1] |
| 615 | 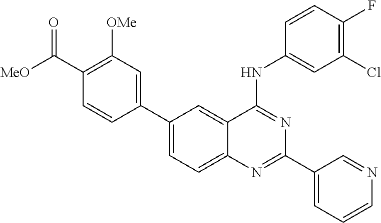 [SMILES: COC(=O)c1ccc(-c2ccc3nc(-c4cccnc4)nc(Nc4ccc(F)c(Cl)c4)c3c2)cc1OC] |
| 616 | 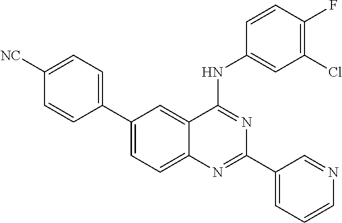 [SMILES: N#Cc1ccc(-c2ccc3nc(-c4cccnc4)nc(Nc4ccc(F)c(Cl)c4)c3c2)cc1] |
| 617 | 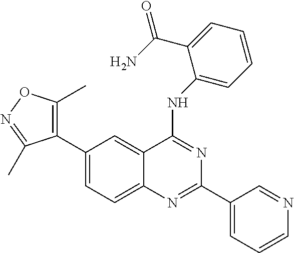 [SMILES: Cc1noc(C)c1-c1ccc2nc(-c3cccnc3)nc(Nc3ccccc3C(N)=O)c2c1] |
| 618 | 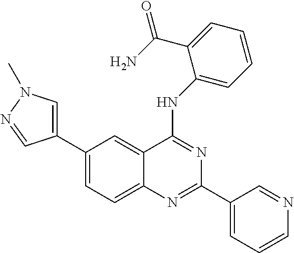 [SMILES: Cn1cc(-c2ccc3nc(-c4cccnc4)nc(Nc4ccccc4C(N)=O)c3c2)cn1] |
| 619 | 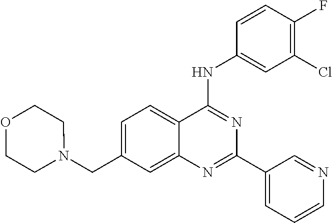 [SMILES: Fc1ccc(Nc2nc(-c3cccnc3)nc3cc(CN4CCOCC4)ccc23)cc1Cl] |
| 620 | 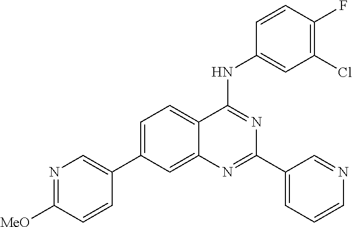 [SMILES: COc1ccc(-c2ccc3c(Nc4ccc(F)c(Cl)c4)nc(-c4cccnc4)nc3c2)cn1] |
| 621 | 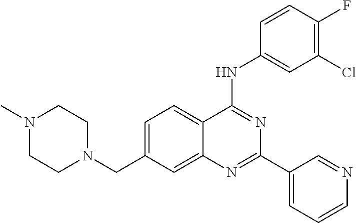 [SMILES: CN1CCN(Cc2ccc3c(Nc4ccc(F)c(Cl)c4)nc(-c4cccnc4)nc3c2)CC1] |
| 622 | 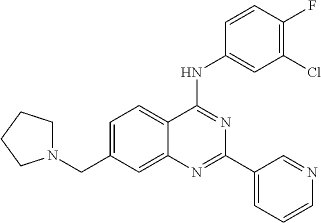 [SMILES: Fc1ccc(Nc2nc(-c3cccnc3)nc3cc(CN4CCCC4)ccc23)cc1Cl] |
| 623 | 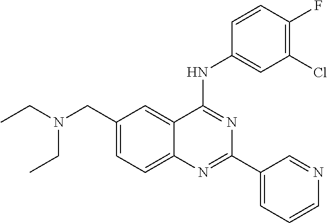 [SMILES: CCN(CC)Cc1ccc2nc(-c3cccnc3)nc(Nc3ccc(F)c(Cl)c3)c2c1] |
| 624 | 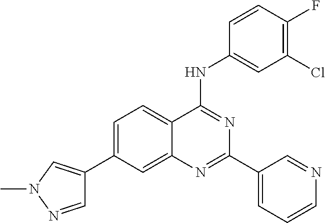 [SMILES: Cn1cc(-c2ccc3c(Nc4ccc(F)c(Cl)c4)nc(-c4cccnc4)nc3c2)cn1] |
| 625 | 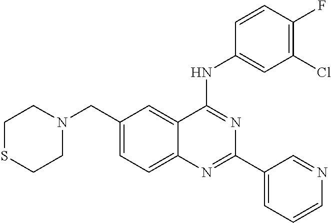 [SMILES: Fc1ccc(Nc2nc(-c3cccnc3)nc3ccc(CN4CCSCC4)cc23)cc1Cl] |
| 626 | 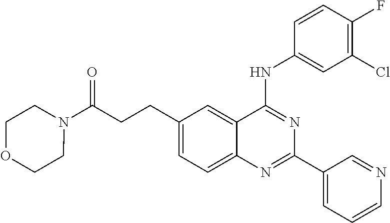 [SMILES: O=C(CCc1ccc2nc(-c3cccnc3)nc(Nc3ccc(F)c(Cl)c3)c2c1)N1CCOCC1] |
| 627 | 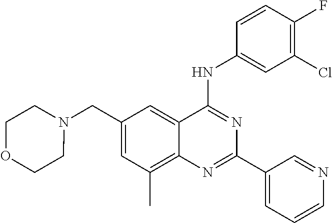 [SMILES: Cc1cc(CN2CCOCC2)cc2c(Nc3ccc(F)c(Cl)c3)nc(-c3cccnc3)nc12] |
| 628 | 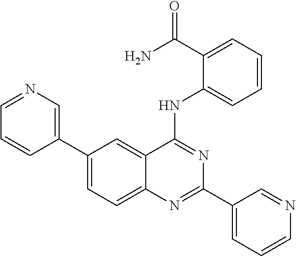 [SMILES: NC(=O)c1ccccc1Nc1nc(-c2cccnc2)nc2ccc(-c3cccnc3)cc12] |
| 629 | 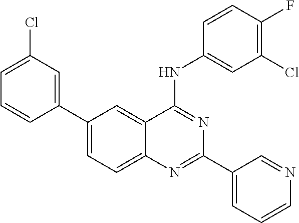 [SMILES: Fc1ccc(Nc2nc(-c3cccnc3)nc3ccc(-c4cccc(Cl)c4)cc23)cc1Cl] |
| 630 | 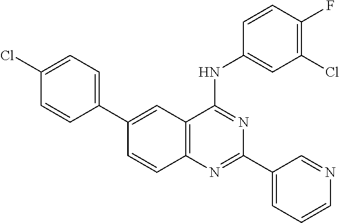 [SMILES: Fc1ccc(Nc2nc(-c3cccnc3)nc3ccc(-c4ccc(Cl)cc4)cc23)cc1Cl] |
| 631 | 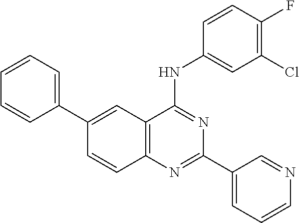 [SMILES: Fc1ccc(Nc2nc(-c3cccnc3)nc3ccc(-c4ccccc4)cc23)cc1Cl] |
| 632 | 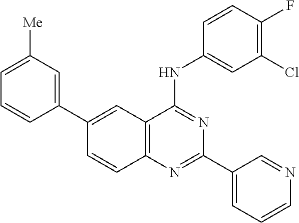 [SMILES: Cc1cccc(-c2ccc3nc(-c4cccnc4)nc(Nc4ccc(F)c(Cl)c4)c3c2)c1] |
| 633 | 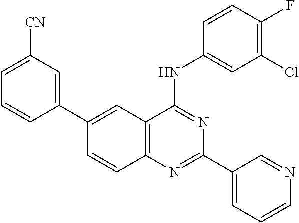 [SMILES: N#Cc1cccc(-c2ccc3nc(-c4cccnc4)nc(Nc4ccc(F)c(Cl)c4)c3c2)c1] |
| 634 | 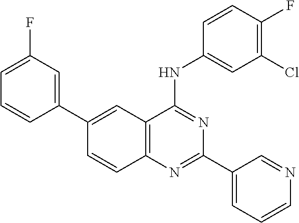 [SMILES: Fc1cccc(-c2ccc3nc(-c4cccnc4)nc(Nc4ccc(F)c(Cl)c4)c3c2)c1] |
| 635 | 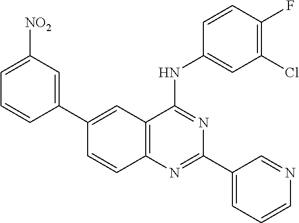 [SMILES: O=[N+]([O-])c1cccc(-c2ccc3nc(-c4cccnc4)nc(Nc4ccc(F)c(Cl)c4)c3c2)c1] |
| 636 | 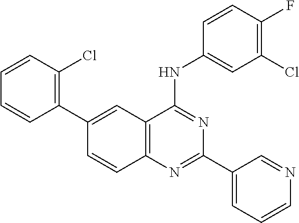 [SMILES: Fc1ccc(Nc2nc(-c3cccnc3)nc3ccc(-c4ccccc4Cl)cc23)cc1Cl] |
| 637 | 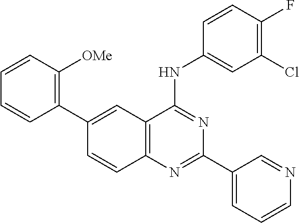 [SMILES: COc1ccccc1-c1ccc2nc(-c3cccnc3)nc(Nc3ccc(F)c(Cl)c3)c2c1] |
| 638 | 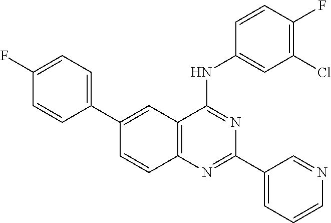 [SMILES: Fc1ccc(-c2ccc3nc(-c4cccnc4)nc(Nc4ccc(F)c(Cl)c4)c3c2)cc1] |
| 639 | 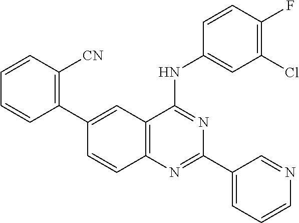 [SMILES: N#Cc1ccccc1-c1ccc2nc(-c3cccnc3)nc(Nc3ccc(F)c(Cl)c3)c2c1] |
| 640 | 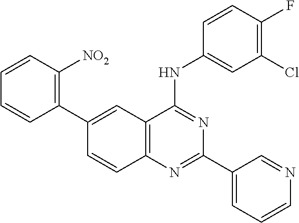 [SMILES: O=[N+]([O-])c1ccccc1-c1ccc2nc(-c3cccnc3)nc(Nc3ccc(F)c(Cl)c3)c2c1] |
| 641 | 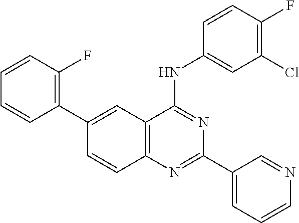 [SMILES: Fc1ccc(Nc2nc(-c3cccnc3)nc3ccc(-c4ccccc4F)cc23)cc1Cl] |
| 642 | 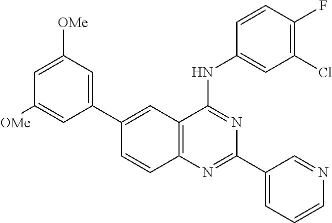 [SMILES: COc1cc(OC)cc(-c2ccc3nc(-c4cccnc4)nc(Nc4ccc(F)c(Cl)c4)c3c2)c1] |
| 643 | 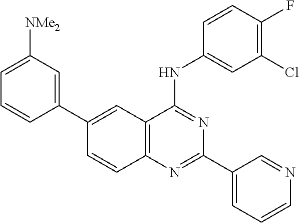 [SMILES: CN(C)c1cccc(-c2ccc3nc(-c4cccnc4)nc(Nc4ccc(F)c(Cl)c4)c3c2)c1] |
| 644 | 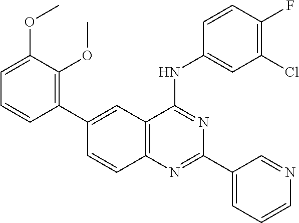 [SMILES: COc1cccc(-c2ccc3nc(-c4cccnc4)nc(Nc4ccc(F)c(Cl)c4)c3c2)c1OC] |
| 645 | 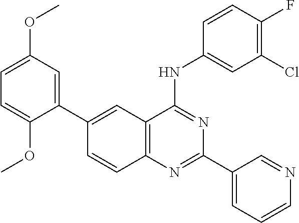 [SMILES: COc1ccc(OC)c(-c2ccc3nc(-c4cccnc4)nc(Nc4ccc(F)c(Cl)c4)c3c2)c1] |
| 646 | 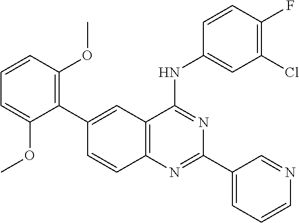 [SMILES: COc1cccc(OC)c1-c1ccc2nc(-c3cccnc3)nc(Nc3ccc(F)c(Cl)c3)c2c1] |
| 647 | 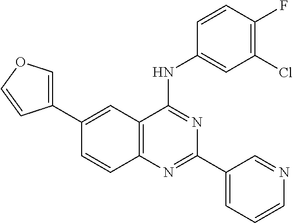 [SMILES: Fc1ccc(Nc2nc(-c3cccnc3)nc3ccc(-c4ccoc4)cc23)cc1Cl] |
| 648 | 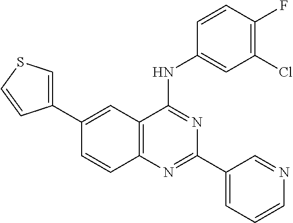 [SMILES: Fc1ccc(Nc2nc(-c3cccnc3)nc3ccc(-c4ccsc4)cc23)cc1Cl] |
| 649 | 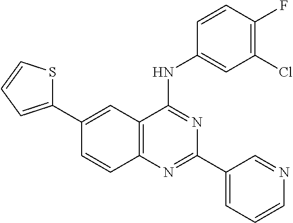 [SMILES: Fc1ccc(Nc2nc(-c3cccnc3)nc3ccc(-c4cccs4)cc23)cc1Cl] |
| 650 | 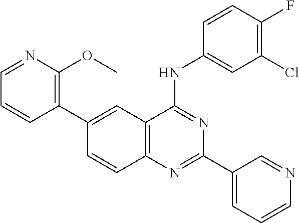 [SMILES: COc1ncccc1-c1ccc2nc(-c3cccnc3)nc(Nc3ccc(F)c(Cl)c3)c2c1] |
| 651 | 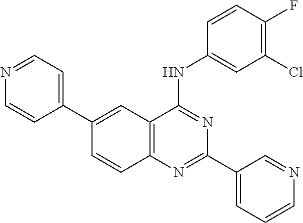 [SMILES: Fc1ccc(Nc2nc(-c3cccnc3)nc3ccc(-c4ccncc4)cc23)cc1Cl] |
| 652 | 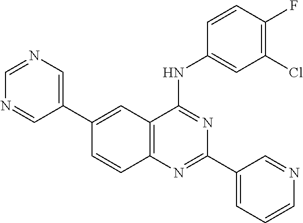 [SMILES: Fc1ccc(Nc2nc(-c3cccnc3)nc3ccc(-c4cncnc4)cc23)cc1Cl] |
| 653 | 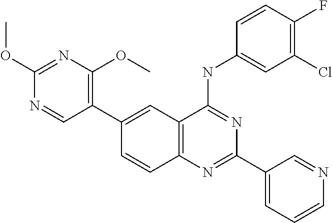 [SMILES: COc1ncc(-c2ccc3nc(-c4cccnc4)nc(Nc4ccc(F)c(Cl)c4)c3c2)c(OC)n1] |
| 654 | 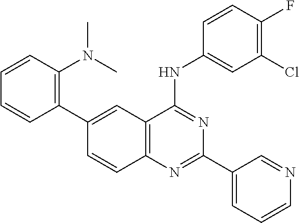 [SMILES: CN(C)c1ccccc1-c1ccc2nc(-c3cccnc3)nc(Nc3ccc(F)c(Cl)c3)c2c1] |
| 655 | 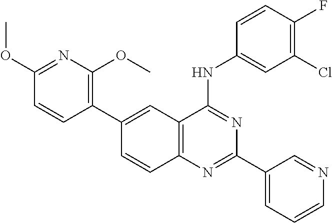 [SMILES: COc1ccc(-c2ccc3nc(-c4cccnc4)nc(Nc4ccc(F)c(Cl)c4)c3c2)c(OC)n1] |
| 656 | 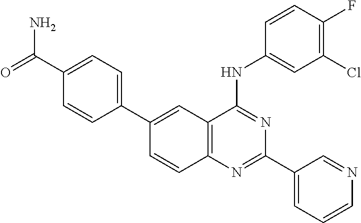 [SMILES: NC(=O)c1ccc(-c2ccc3nc(-c4cccnc4)nc(Nc4ccc(F)c(Cl)c4)c3c2)cc1] |
| 657 | 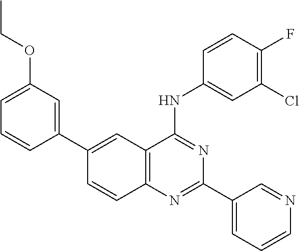 [SMILES: CCOc1cccc(-c2ccc3nc(-c4cccnc4)nc(Nc4ccc(F)c(Cl)c4)c3c2)c1] |
| 658 | 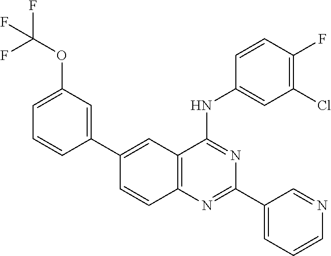 [SMILES: Fc1ccc(Nc2nc(-c3cccnc3)nc3ccc(-c4cccc(OC(F)(F)F)c4)cc23)cc1Cl] |
| 659 | 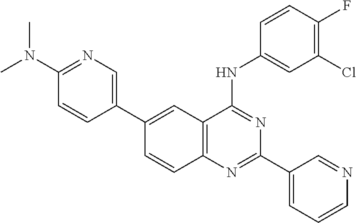 [SMILES: CN(C)c1ccc(-c2ccc3nc(-c4cccnc4)nc(Nc4ccc(F)c(Cl)c4)c3c2)cn1] |
| 660 | 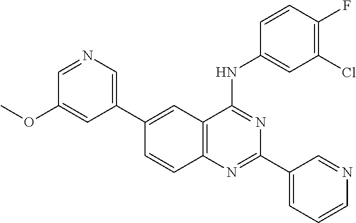 [SMILES: COc1cncc(-c2ccc3nc(-c4cccnc4)nc(Nc4ccc(F)c(Cl)c4)c3c2)c1] |
| 661 | 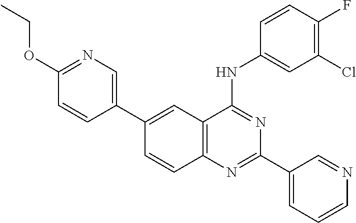 [SMILES: CCOc1ccc(-c2ccc3nc(-c4cccnc4)nc(Nc4ccc(F)c(Cl)c4)c3c2)cn1] |
| 662 | 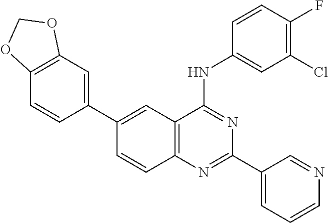 [SMILES: Fc1ccc(Nc2nc(-c3cccnc3)nc3ccc(-c4ccc5c(c4)OCO5)cc23)cc1Cl] |
| 663 | 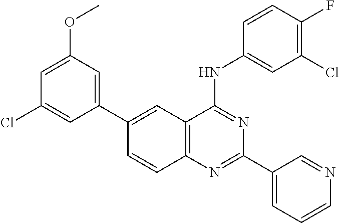 [SMILES: COc1cc(Cl)cc(-c2ccc3nc(-c4cccnc4)nc(Nc4ccc(F)c(Cl)c4)c3c2)c1] |
| 664 | 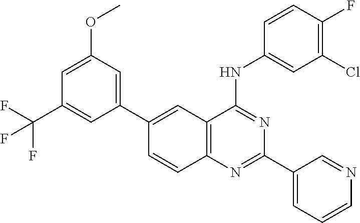 [SMILES: COc1cc(-c2ccc3nc(-c4cccnc4)nc(Nc4ccc(F)c(Cl)c4)c3c2)cc(C(F)(F)F)c1] |
| 665 | 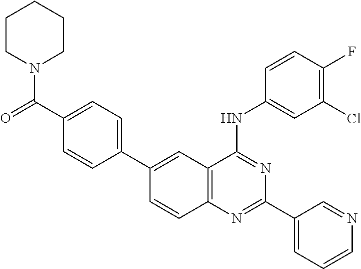 [SMILES: O=C(c1ccc(-c2ccc3nc(-c4cccnc4)nc(Nc4ccc(F)c(Cl)c4)c3c2)cc1)N1CCCCC1] |
| 666 | 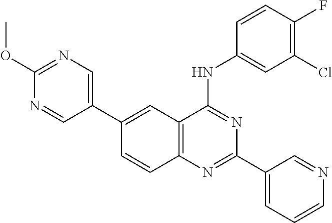 [SMILES: COc1ncc(-c2ccc3nc(-c4cccnc4)nc(Nc4ccc(F)c(Cl)c4)c3c2)cn1] |
| 667 | 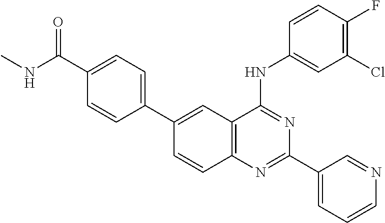 [SMILES: CNC(=O)c1ccc(-c2ccc3nc(-c4cccnc4)nc(Nc4ccc(F)c(Cl)c4)c3c2)cc1] |
| 668 | 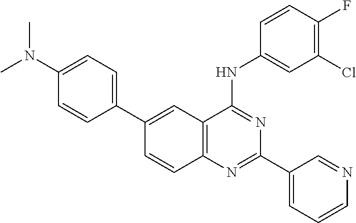 [SMILES: CN(C)c1ccc(-c2ccc3nc(-c4cccnc4)nc(Nc4ccc(F)c(Cl)c4)c3c2)cc1] |
| 669 | 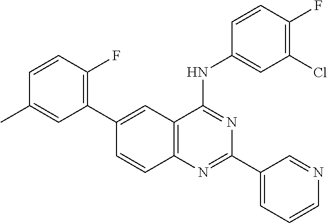 [SMILES: Cc1ccc(F)c(-c2ccc3nc(-c4cccnc4)nc(Nc4ccc(F)c(Cl)c4)c3c2)c1] |
| 670 | 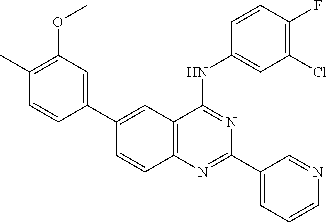 [SMILES: COc1cc(-c2ccc3nc(-c4cccnc4)nc(Nc4ccc(F)c(Cl)c4)c3c2)ccc1C] |
| 671 | 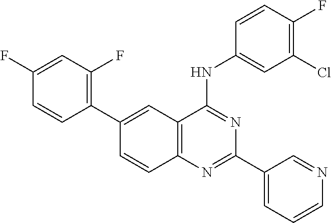 [SMILES: Fc1ccc(-c2ccc3nc(-c4cccnc4)nc(Nc4ccc(F)c(Cl)c4)c3c2)c(F)c1] |
| 672 | 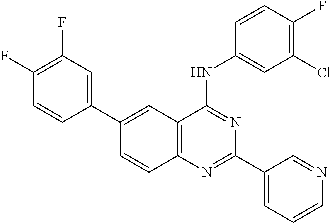 [SMILES: Fc1ccc(-c2ccc3nc(-c4cccnc4)nc(Nc4ccc(F)c(Cl)c4)c3c2)cc1F] |
| 673 | 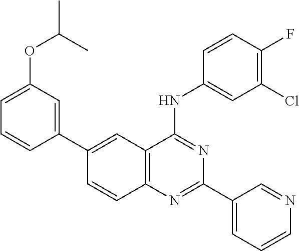 [SMILES: CC(C)Oc1cccc(-c2ccc3nc(-c4cccnc4)nc(Nc4ccc(F)c(Cl)c4)c3c2)c1] |
| 674 | 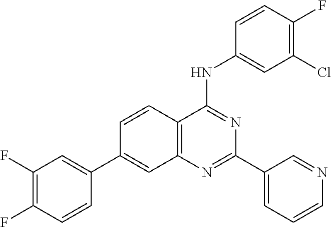 [SMILES: Fc1ccc(-c2ccc3c(Nc4ccc(F)c(Cl)c4)nc(-c4cccnc4)nc3c2)cc1F] |
| 675 | 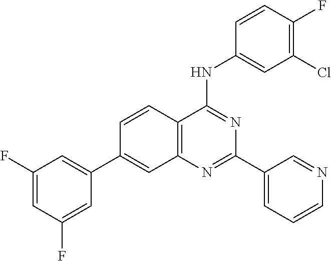 [SMILES: Fc1cc(F)cc(-c2ccc3c(Nc4ccc(F)c(Cl)c4)nc(-c4cccnc4)nc3c2)c1] |
| 676 | 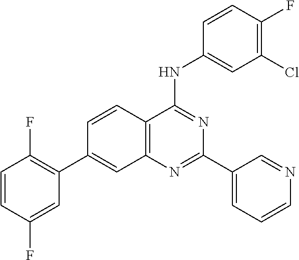 [SMILES: Fc1ccc(F)c(-c2ccc3c(Nc4ccc(F)c(Cl)c4)nc(-c4cccnc4)nc3c2)c1] |
| 677 | 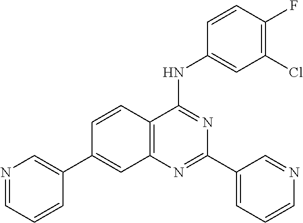 [SMILES: Fc1ccc(Nc2nc(-c3cccnc3)nc3cc(-c4cccnc4)ccc23)cc1Cl] |
| 678 | 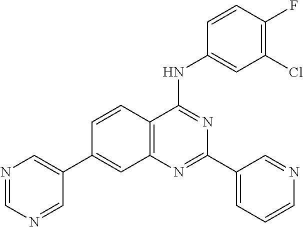 [SMILES: Fc1ccc(Nc2nc(-c3cccnc3)nc3cc(-c4cncnc4)ccc23)cc1Cl] |
| 679 | 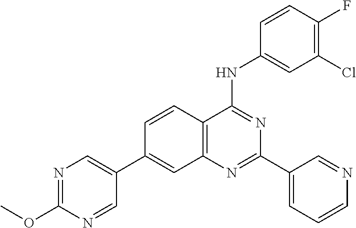 [SMILES: COc1ncc(-c2ccc3c(Nc4ccc(F)c(Cl)c4)nc(-c4cccnc4)nc3c2)cn1] |
| 680 | 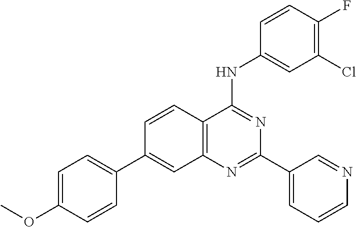 [SMILES: COc1ccc(-c2ccc3c(Nc4ccc(F)c(Cl)c4)nc(-c4cccnc4)nc3c2)cc1] |
| 681 | 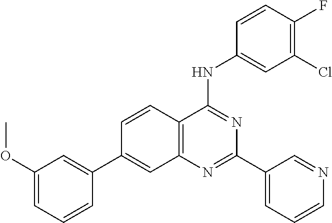 [SMILES: COc1cccc(-c2ccc3c(Nc4ccc(F)c(Cl)c4)nc(-c4cccnc4)nc3c2)c1] |
| 682 | 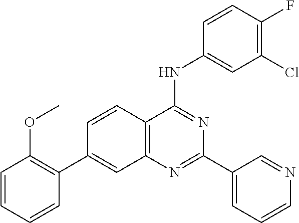 [SMILES: COc1ccccc1-c1ccc2c(Nc3ccc(F)c(Cl)c3)nc(-c3cccnc3)nc2c1] |
| 683 | 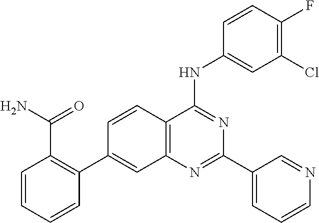 [SMILES: NC(=O)c1ccccc1-c1ccc2c(Nc3ccc(F)c(Cl)c3)nc(-c3cccnc3)nc2c1] |
| 684 | 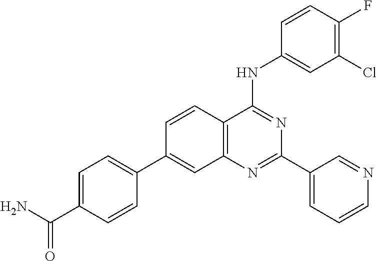 [SMILES: NC(=O)c1ccc(-c2ccc3c(Nc4ccc(F)c(Cl)c4)nc(-c4cccnc4)nc3c2)cc1] |
| 685 | 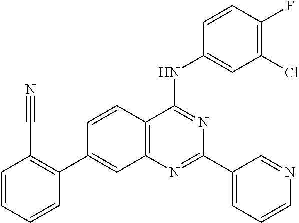 [SMILES: N#Cc1ccccc1-c1ccc2c(Nc3ccc(F)c(Cl)c3)nc(-c3cccnc3)nc2c1] |
| 686 | 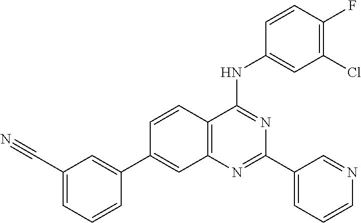 [SMILES: N#Cc1cccc(-c2ccc3c(Nc4ccc(F)c(Cl)c4)nc(-c4cccnc4)nc3c2)c1] |
| 687 | 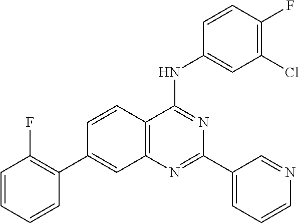 [SMILES: Fc1ccc(Nc2nc(-c3cccnc3)nc3cc(-c4ccccc4F)ccc23)cc1Cl] |
| 688 | 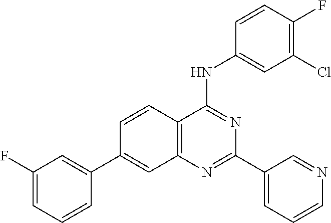 [SMILES: Fc1cccc(-c2ccc3c(Nc4ccc(F)c(Cl)c4)nc(-c4cccnc4)nc3c2)c1] |
| 689 | 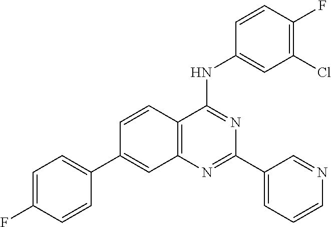 [SMILES: Fc1ccc(-c2ccc3c(Nc4ccc(F)c(Cl)c4)nc(-c4cccnc4)nc3c2)cc1] |
| 690 | 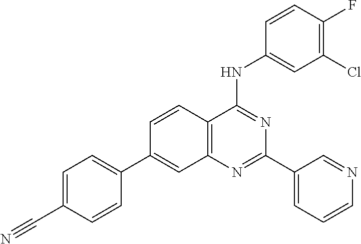 [SMILES: N#Cc1ccc(-c2ccc3c(Nc4ccc(F)c(Cl)c4)nc(-c4cccnc4)nc3c2)cc1] |
| 691 | 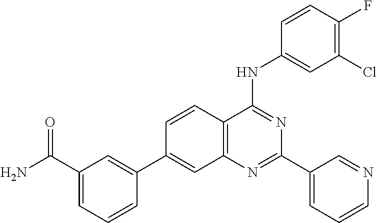 [SMILES: NC(=O)c1cccc(-c2ccc3c(Nc4ccc(F)c(Cl)c4)nc(-c4cccnc4)nc3c2)c1] |
| 692 | 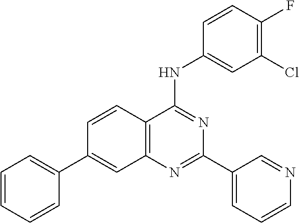 [SMILES: Fc1ccc(Nc2nc(-c3cccnc3)nc3cc(-c4ccccc4)ccc23)cc1Cl] |
| 693 | 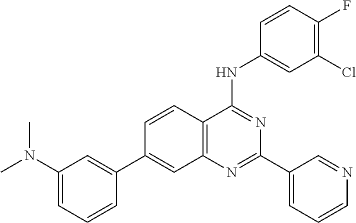 [SMILES: CN(C)c1cccc(-c2ccc3c(Nc4ccc(F)c(Cl)c4)nc(-c4cccnc4)nc3c2)c1] |
| 694 | 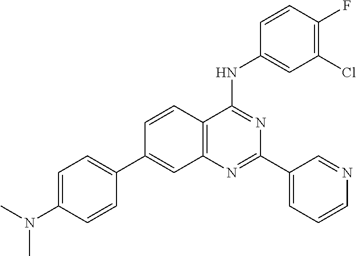 [SMILES: CN(C)c1ccc(-c2ccc3c(Nc4ccc(F)c(Cl)c4)nc(-c4cccnc4)nc3c2)cc1] |
| 695 | 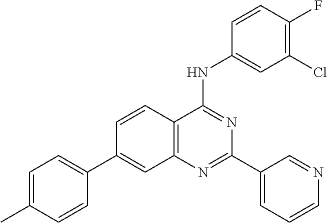 [SMILES: Cc1ccc(-c2ccc3c(Nc4ccc(F)c(Cl)c4)nc(-c4cccnc4)nc3c2)cc1] |
| 696 | 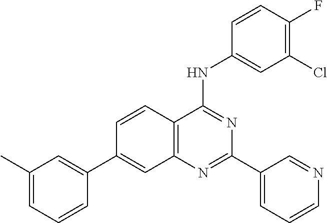 [SMILES: Cc1cccc(-c2ccc3c(Nc4ccc(F)c(Cl)c4)nc(-c4cccnc4)nc3c2)c1] |
| 697 | 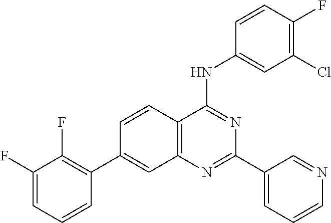 [SMILES: Fc1ccc(Nc2nc(-c3cccnc3)nc3cc(-c4cccc(F)c4F)ccc23)cc1Cl] |
| 698 | 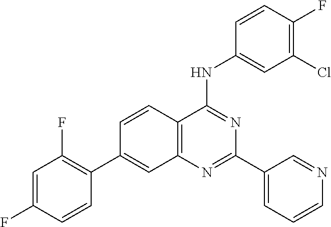 [SMILES: Fc1ccc(-c2ccc3c(Nc4ccc(F)c(Cl)c4)nc(-c4cccnc4)nc3c2)c(F)c1] |
| 699 | 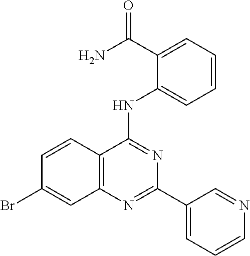 [SMILES: NC(=O)c1ccccc1Nc1nc(-c2cccnc2)nc2cc(Br)ccc12] |
| 700 | 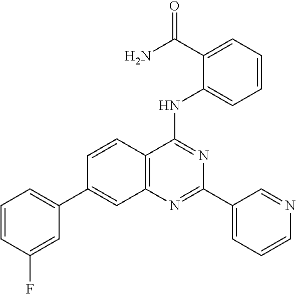 [SMILES: NC(=O)c1ccccc1Nc1nc(-c2cccnc2)nc2cc(-c3cccc(F)c3)ccc12] |
| 701 | 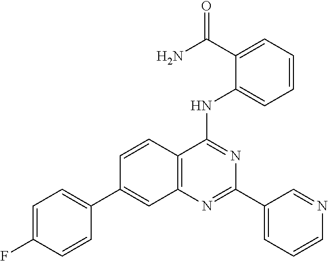 [SMILES: NC(=O)c1ccccc1Nc1nc(-c2cccnc2)nc2cc(-c3ccc(F)cc3)ccc12] |
| 702 | 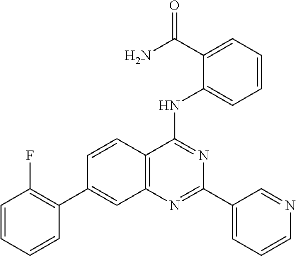 [SMILES: NC(=O)c1ccccc1Nc1nc(-c2cccnc2)nc2cc(-c3ccccc3F)ccc12] |
| 703 | 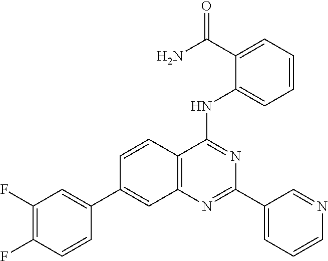 [SMILES: NC(=O)c1ccccc1Nc1nc(-c2cccnc2)nc2cc(-c3ccc(F)c(F)c3)ccc12] |
| 704 | 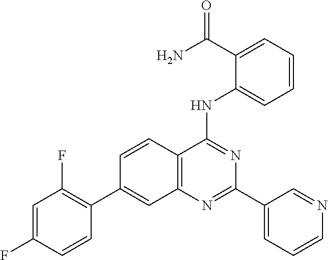 [SMILES: NC(=O)c1ccccc1Nc1nc(-c2cccnc2)nc2cc(-c3ccc(F)cc3F)ccc12] |
| 705 | 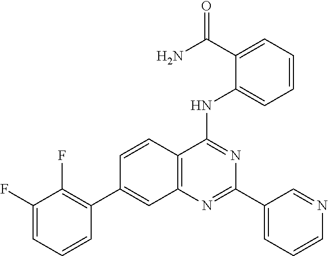 [SMILES: NC(=O)c1ccccc1Nc1nc(-c2cccnc2)nc2cc(-c3cccc(F)c3F)ccc12] |
| 706 | 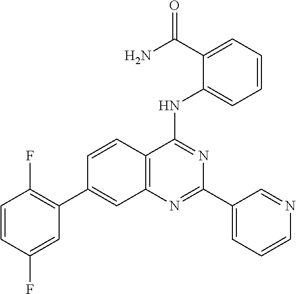 [SMILES: NC(=O)c1ccccc1Nc1nc(-c2cccnc2)nc2cc(-c3cc(F)ccc3F)ccc12] |
| 707 | 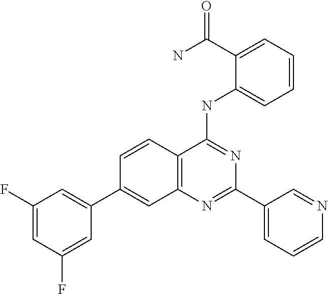 [SMILES: NC(=O)c1ccccc1Nc1nc(-c2cccnc2)nc2cc(-c3cc(F)cc(F)c3)ccc12] |
| 708 | 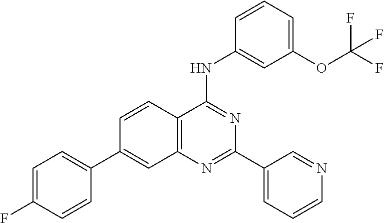 [SMILES: Fc1ccc(-c2ccc3c(Nc4cccc(OC(F)(F)F)c4)nc(-c4cccnc4)nc3c2)cc1] |
| 709 | 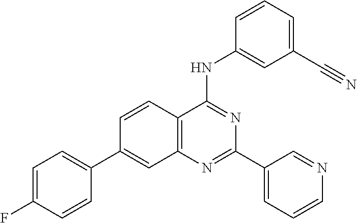 [SMILES: N#Cc1cccc(Nc2nc(-c3cccnc3)nc3cc(-c4ccc(F)cc4)ccc23)c1] |
| 710 | 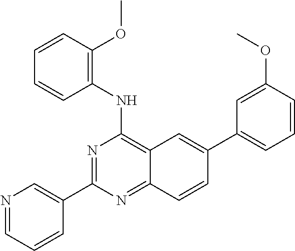 [SMILES: COc1cccc(-c2ccc3nc(-c4cccnc4)nc(Nc4ccccc4OC)c3c2)c1] |
| 711 | 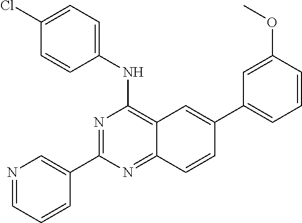 [SMILES: COc1cccc(-c2ccc3nc(-c4cccnc4)nc(Nc4ccc(Cl)cc4)c3c2)c1] |
| 712 | 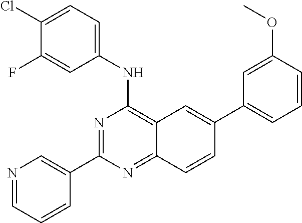 [SMILES: COc1cccc(-c2ccc3nc(-c4cccnc4)nc(Nc4ccc(Cl)c(F)c4)c3c2)c1] |
| 713 | 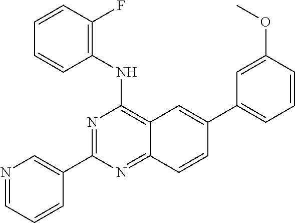 [SMILES: COc1cccc(-c2ccc3nc(-c4cccnc4)nc(Nc4ccccc4F)c3c2)c1] |
| 714 | 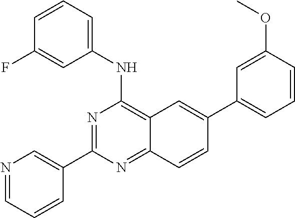 [SMILES: COc1cccc(-c2ccc3nc(-c4cccnc4)nc(Nc4cccc(F)c4)c3c2)c1] |
| 715 | 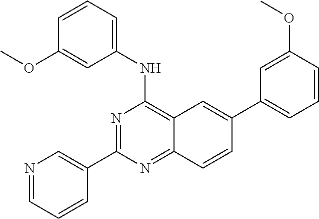 [SMILES: COc1cccc(Nc2nc(-c3cccnc3)nc3ccc(-c4cccc(OC)c4)cc23)c1] |
| 716 | 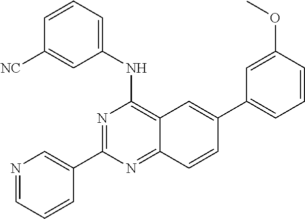 [SMILES: COc1cccc(-c2ccc3nc(-c4cccnc4)nc(Nc4cccc(C#N)c4)c3c2)c1] |
| 717 | 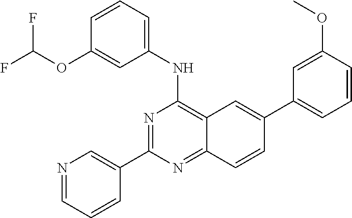 [SMILES: COc1cccc(-c2ccc3nc(-c4cccnc4)nc(Nc4cccc(OC(F)F)c4)c3c2)c1] |
| 718 | 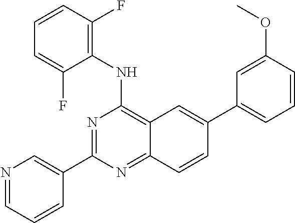 [SMILES: COc1cccc(-c2ccc3nc(-c4cccnc4)nc(Nc4c(F)cccc4F)c3c2)c1] |
| 719 | 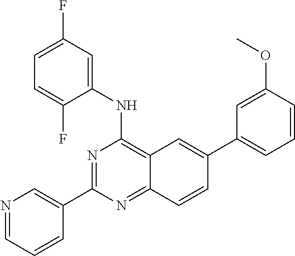 [SMILES: COc1cccc(-c2ccc3nc(-c4cccnc4)nc(Nc4cc(F)ccc4F)c3c2)c1] |
| 720 | 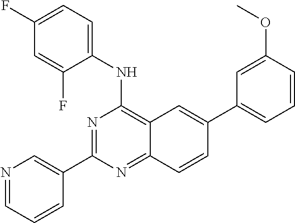 [SMILES: COc1cccc(-c2ccc3nc(-c4cccnc4)nc(Nc4ccc(F)cc4F)c3c2)c1] |
| 721 | 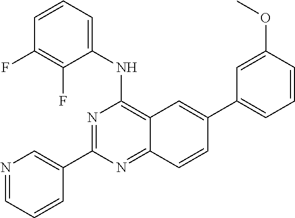 [SMILES: COc1cccc(-c2ccc3nc(-c4cccnc4)nc(Nc4cccc(F)c4F)c3c2)c1] |
| 722 | 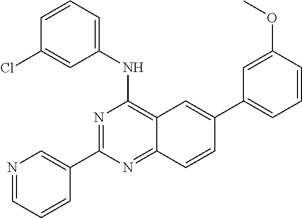 [SMILES: COc1cccc(-c2ccc3nc(-c4cccnc4)nc(Nc4cccc(Cl)c4)c3c2)c1] |
| 723 | 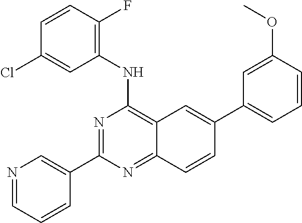 [SMILES: COc1cccc(-c2ccc3nc(-c4cccnc4)nc(Nc4cc(Cl)ccc4F)c3c2)c1] |
| 724 | 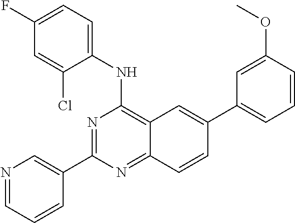 [SMILES: COc1cccc(-c2ccc3nc(-c4cccnc4)nc(Nc4ccc(F)cc4Cl)c3c2)c1] |
| 725 | 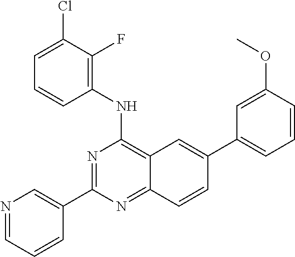 [SMILES: COc1cccc(-c2ccc3nc(-c4cccnc4)nc(Nc4cccc(Cl)c4F)c3c2)c1] |
| 726 | 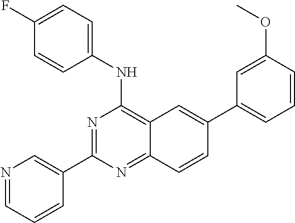 [SMILES: COc1cccc(-c2ccc3nc(-c4cccnc4)nc(Nc4ccc(F)cc4)c3c2)c1] |
| 727 | 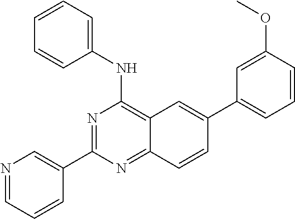 [SMILES: COc1cccc(-c2ccc3nc(-c4cccnc4)nc(Nc4ccccc4)c3c2)c1] |
| 728 | 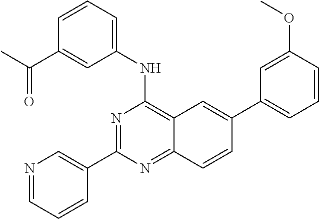 [SMILES: COc1cccc(-c2ccc3nc(-c4cccnc4)nc(Nc4cccc(C(C)=O)c4)c3c2)c1] |
| 729 | 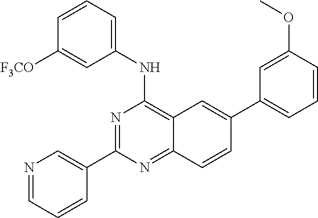 [SMILES: COc1cccc(-c2ccc3nc(-c4cccnc4)nc(Nc4cccc(OC(F)(F)F)c4)c3c2)c1] |
| 730 | 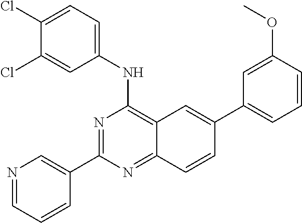 [SMILES: COc1cccc(-c2ccc3nc(-c4cccnc4)nc(Nc4ccc(Cl)c(Cl)c4)c3c2)c1] |
| 731 | 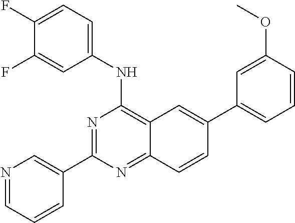 [SMILES: COc1cccc(-c2ccc3nc(-c4cccnc4)nc(Nc4ccc(F)c(F)c4)c3c2)c1] |
| 732 | 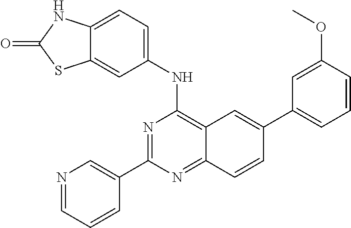 [SMILES: COc1cccc(-c2ccc3nc(-c4cccnc4)nc(Nc4ccc5[nH]c(=O)sc5c4)c3c2)c1] |
| 733 | 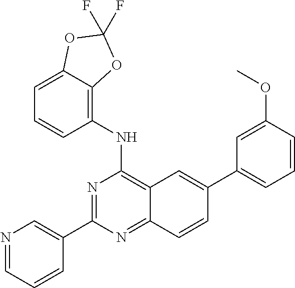 [SMILES: COc1cccc(-c2ccc3nc(-c4cccnc4)nc(Nc4cccc5c4OC(F)(F)O5)c3c2)c1] |
| 734 | 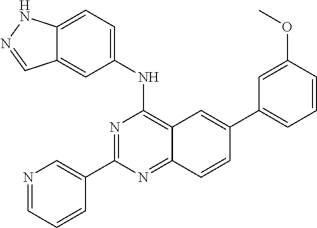 [SMILES: COc1cccc(-c2ccc3nc(-c4cccnc4)nc(Nc4ccc5[nH]ncc5c4)c3c2)c1] |
| 735 | 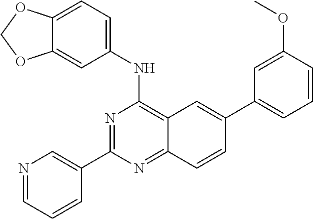 [SMILES: COc1cccc(-c2ccc3nc(-c4cccnc4)nc(Nc4ccc5c(c4)OCO5)c3c2)c1] |
| 736 | 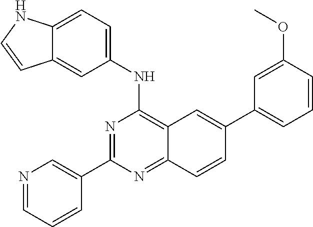 [SMILES: COc1cccc(-c2ccc3nc(-c4cccnc4)nc(Nc4ccc5[nH]ccc5c4)c3c2)c1] |
| 737 | 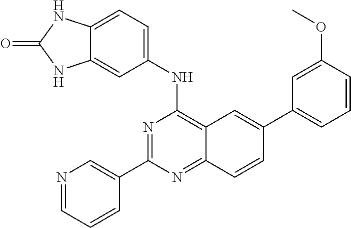 [SMILES: COc1cccc(-c2ccc3nc(-c4cccnc4)nc(Nc4ccc5[nH]c(=O)[nH]c5c4)c3c2)c1] |
| 738 | 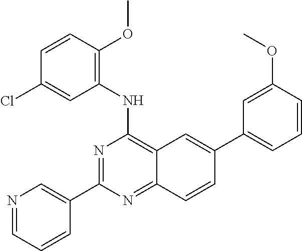 [SMILES: COc1cccc(-c2ccc3nc(-c4cccnc4)nc(Nc4cc(Cl)ccc4OC)c3c2)c1] |
| 739 | 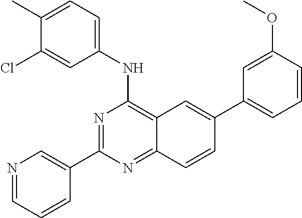 [SMILES: COc1cccc(-c2ccc3nc(-c4cccnc4)nc(Nc4ccc(C)c(Cl)c4)c3c2)c1] |
| 740 | 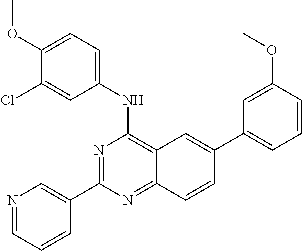 [SMILES: COc1cccc(-c2ccc3nc(-c4cccnc4)nc(Nc4ccc(OC)c(Cl)c4)c3c2)c1] |
| 741 | 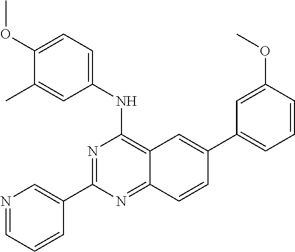 [SMILES: COc1cccc(-c2ccc3nc(-c4cccnc4)nc(Nc4ccc(OC)c(C)c4)c3c2)c1] |
| 742 | 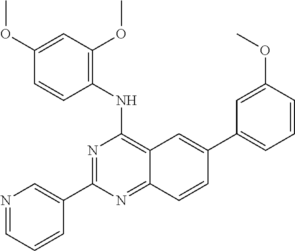 [SMILES: COc1cccc(-c2ccc3nc(-c4cccnc4)nc(Nc4ccc(OC)cc4OC)c3c2)c1] |
| 743 | 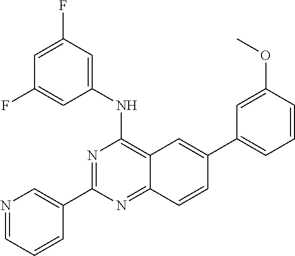 [SMILES: COc1cccc(-c2ccc3nc(-c4cccnc4)nc(Nc4cc(F)cc(F)c4)c3c2)c1] |
| 744 | 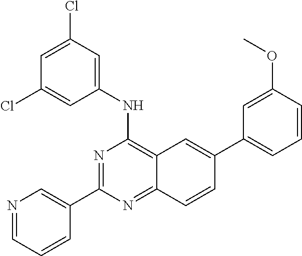 [SMILES: COc1cccc(-c2ccc3nc(-c4cccnc4)nc(Nc4cc(Cl)cc(Cl)c4)c3c2)c1] |
| 745 | 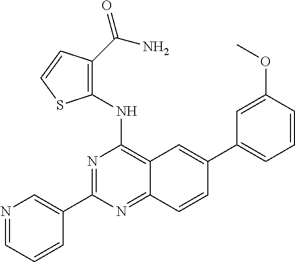 [SMILES: COc1cccc(-c2ccc3nc(-c4cccnc4)nc(Nc4sccc4C(N)=O)c3c2)c1] |
| 746 | 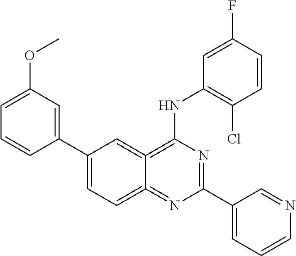 |
| 747 | 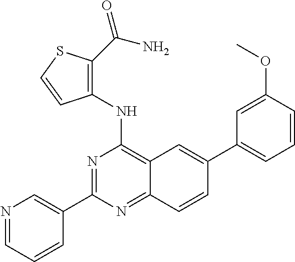 [SMILES: COc1cccc(-c2ccc3nc(-c4cccnc4)nc(Nc4ccsc4C(N)=O)c3c2)c1] |
| 748 | 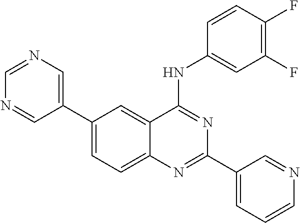 [SMILES: Fc1ccc(Nc2nc(-c3cccnc3)nc3ccc(-c4cncnc4)cc23)cc1F] |
| 749 | 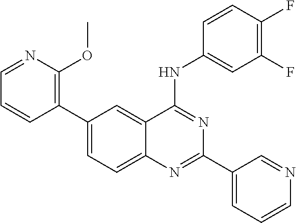 [SMILES: COc1ncccc1-c1ccc2nc(-c3cccnc3)nc(Nc3ccc(F)c(F)c3)c2c1] |
| 750 | 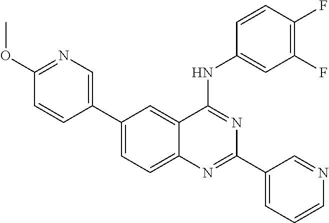 [SMILES: COc1ccc(-c2ccc3nc(-c4cccnc4)nc(Nc4ccc(F)c(F)c4)c3c2)cn1] |
| 751 | 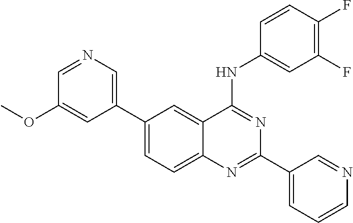 [SMILES: COc1cncc(-c2ccc3nc(-c4cccnc4)nc(Nc4ccc(F)c(F)c4)c3c2)c1] |
| 752 | 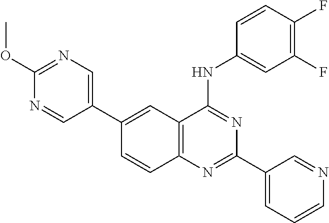 [SMILES: COc1ncc(-c2ccc3nc(-c4cccnc4)nc(Nc4ccc(F)c(F)c4)c3c2)cn1] |
| 753 | 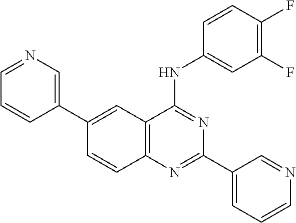 [SMILES: Fc1ccc(Nc2nc(-c3cccnc3)nc3ccc(-c4cccnc4)cc23)cc1F] |
| 754 | 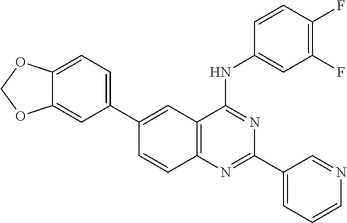 [SMILES: Fc1ccc(Nc2nc(-c3cccnc3)nc3ccc(-c4ccc5c(c4)OCO5)cc23)cc1F] |
| 755 | 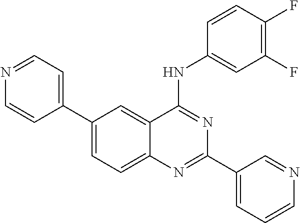 [SMILES: Fc1ccc(Nc2nc(-c3cccnc3)nc3ccc(-c4ccncc4)cc23)cc1F] |
| 756 | 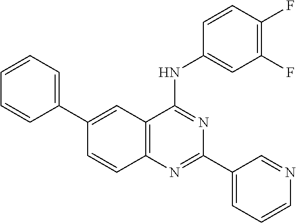 [SMILES: Fc1ccc(Nc2nc(-c3cccnc3)nc3ccc(-c4ccccc4)cc23)cc1F] |
| 757 | 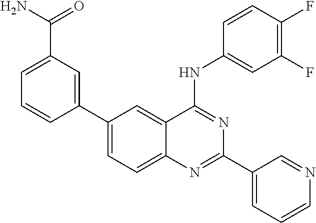 [SMILES: NC(=O)c1cccc(-c2ccc3nc(-c4cccnc4)nc(Nc4ccc(F)c(F)c4)c3c2)c1] |
| 758 | 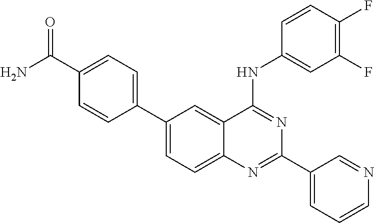 [SMILES: NC(=O)c1ccc(-c2ccc3nc(-c4cccnc4)nc(Nc4ccc(F)c(F)c4)c3c2)cc1] |
| 759 | 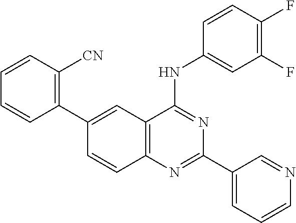 [SMILES: N#Cc1ccccc1-c1ccc2nc(-c3cccnc3)nc(Nc3ccc(F)c(F)c3)c2c1] |
| 760 | 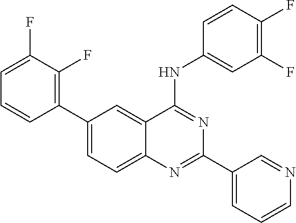 [SMILES: Fc1ccc(Nc2nc(-c3cccnc3)nc3ccc(-c4cccc(F)c4F)cc23)cc1F] |
| 761 | 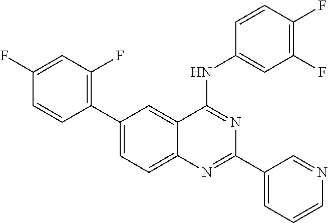 [SMILES: Fc1ccc(-c2ccc3nc(-c4cccnc4)nc(Nc4ccc(F)c(F)c4)c3c2)c(F)c1] |
| 762 | 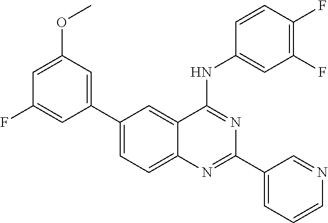 [SMILES: COc1cc(F)cc(-c2ccc3nc(-c4cccnc4)nc(Nc4ccc(F)c(F)c4)c3c2)c1] |
| 763 | 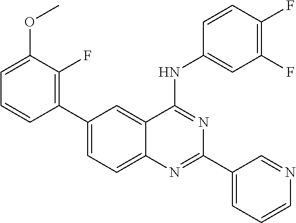 [SMILES: COc1cccc(-c2ccc3nc(-c4cccnc4)nc(Nc4ccc(F)c(F)c4)c3c2)c1F] |
| 764 | 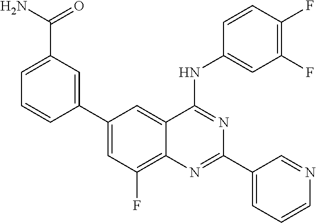 [SMILES: NC(=O)c1cccc(-c2cc(F)c3nc(-c4cccnc4)nc(Nc4ccc(F)c(F)c4)c3c2)c1] |
| 765 | 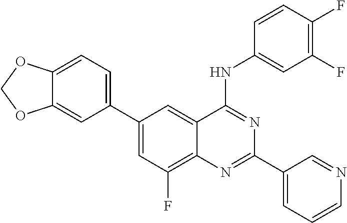 [SMILES: Fc1ccc(Nc2nc(-c3cccnc3)nc3c(F)cc(-c4ccc5c(c4)OCO5)cc23)cc1F] |
| 766 | 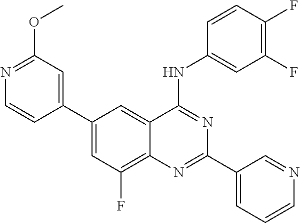 [SMILES: COc1cc(-c2cc(F)c3nc(-c4cccnc4)nc(Nc4ccc(F)c(F)c4)c3c2)ccn1] |
| 767 | 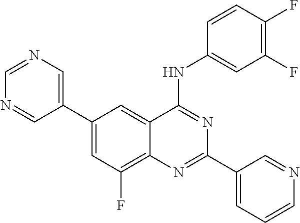 [SMILES: Fc1ccc(Nc2nc(-c3cccnc3)nc3c(F)cc(-c4cncnc4)cc23)cc1F] |
| 768 | 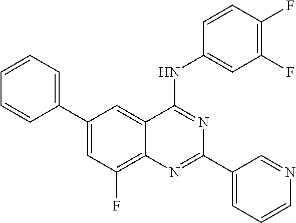 [SMILES: Fc1ccc(Nc2nc(-c3cccnc3)nc3c(F)cc(-c4ccccc4)cc23)cc1F] |
| 769 | 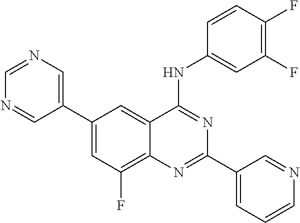 [SMILES: Fc1ccc(Nc2nc(-c3cccnc3)nc3c(F)cc(-c4cncnc4)cc23)cc1F] |
| 770 | 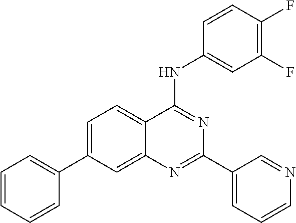 [SMILES: Fc1ccc(Nc2nc(-c3cccnc3)nc3cc(-c4ccccc4)ccc23)cc1F] |
| 771 | 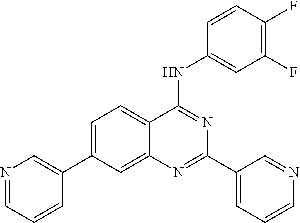 [SMILES: Fc1ccc(Nc2nc(-c3cccnc3)nc3cc(-c4cccnc4)ccc23)cc1F] |
| 772 | 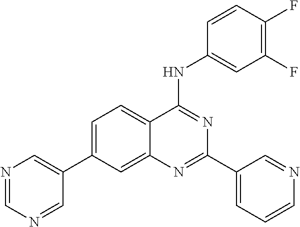 [SMILES: Fc1ccc(Nc2nc(-c3cccnc3)nc3cc(-c4cncnc4)ccc23)cc1F] |
| 773 | 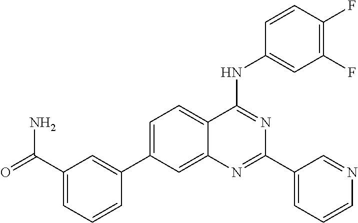 [SMILES: NC(=O)c1cccc(-c2ccc3c(Nc4ccc(F)c(F)c4)nc(-c4cccnc4)nc3c2)c1] |
| 774 | 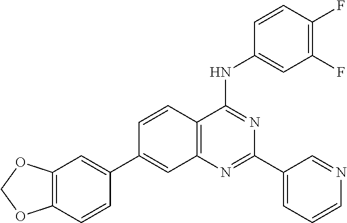 [SMILES: Fc1ccc(Nc2nc(-c3cccnc3)nc3cc(-c4ccc5c(c4)OCO5)ccc23)cc1F] |
| 775 | 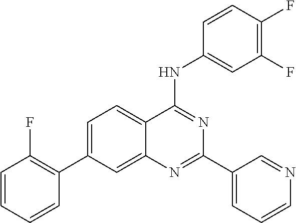 [SMILES: Fc1ccc(Nc2nc(-c3cccnc3)nc3cc(-c4ccccc4F)ccc23)cc1F] |
| 776 | 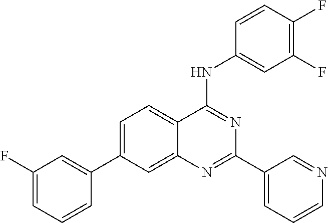 [SMILES: Fc1cccc(-c2ccc3c(Nc4ccc(F)c(F)c4)nc(-c4cccnc4)nc3c2)c1] |
| 777 | 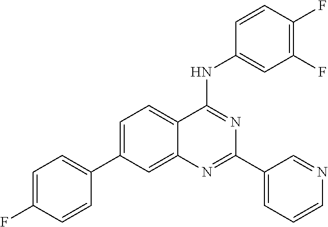 [SMILES: Fc1ccc(-c2ccc3c(Nc4ccc(F)c(F)c4)nc(-c4cccnc4)nc3c2)cc1] |
| 778 | 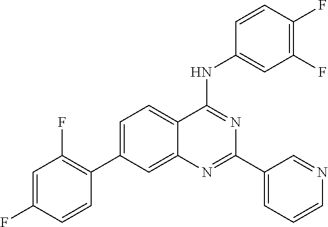 [SMILES: Fc1ccc(-c2ccc3c(Nc4ccc(F)c(F)c4)nc(-c4cccnc4)nc3c2)c(F)c1] |
| 779 | 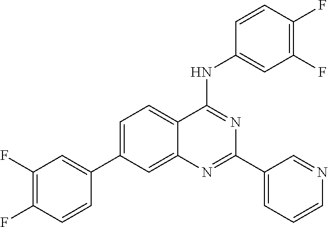 [SMILES: Fc1ccc(Nc2nc(-c3cccnc3)nc3cc(-c4ccc(F)c(F)c4)ccc23)cc1F] |
| 780 | 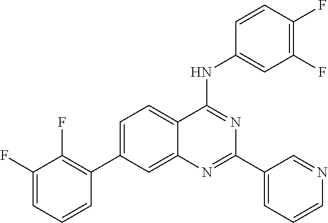 [SMILES: Fc1ccc(Nc2nc(-c3cccnc3)nc3cc(-c4cccc(F)c4F)ccc23)cc1F] |
| 781 | 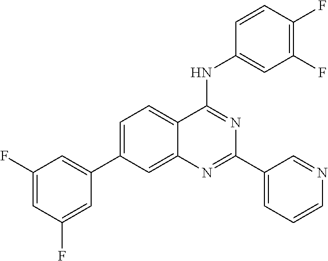 [SMILES: Fc1cc(F)cc(-c2ccc3c(Nc4ccc(F)c(F)c4)nc(-c4cccnc4)nc3c2)c1] |
| 782 | 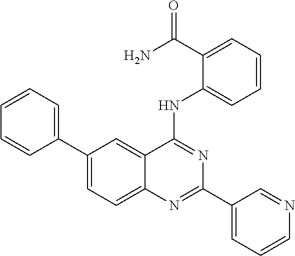 [SMILES: NC(=O)c1ccccc1Nc1nc(-c2cccnc2)nc2ccc(-c3ccccc3)cc12] |
| 783 | 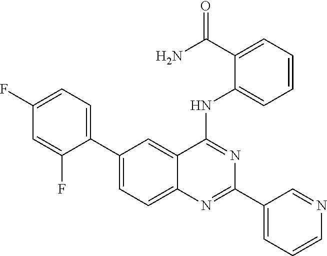 [SMILES: NC(=O)c1ccccc1Nc1nc(-c2cccnc2)nc2ccc(-c3ccc(F)cc3F)cc12] |
| 784 | 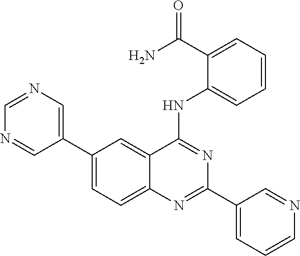 [SMILES: NC(=O)c1ccccc1Nc1nc(-c2cccnc2)nc2ccc(-c3cncnc3)cc12] |
| 785 | 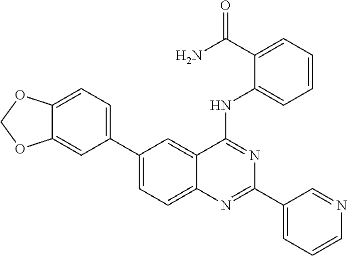 [SMILES: NC(=O)c1ccccc1Nc1nc(-c2cccnc2)nc2ccc(-c3ccc4c(c3)OCO4)cc12] |
| 786 | 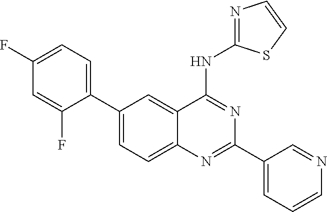 [SMILES: Fc1ccc(-c2ccc3nc(-c4cccnc4)nc(Nc4nccs4)c3c2)c(F)c1] |
| 787 | 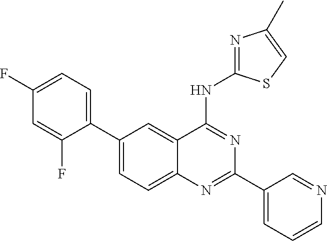 [SMILES: Cc1csc(Nc2nc(-c3cccnc3)nc3ccc(-c4ccc(F)cc4F)cc23)n1] |
| 788 | 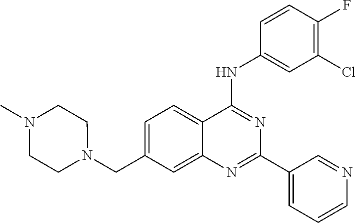 [SMILES: CN1CCN(Cc2ccc3c(Nc4ccc(F)c(Cl)c4)nc(-c4cccnc4)nc3c2)CC1] |
| 789 | 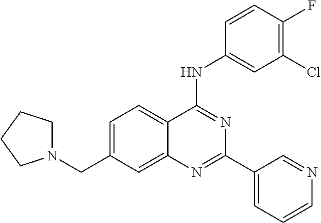 [SMILES: Fc1ccc(Nc2nc(-c3cccnc3)nc3cc(CN4CCCC4)ccc23)cc1Cl] |
| 790 | 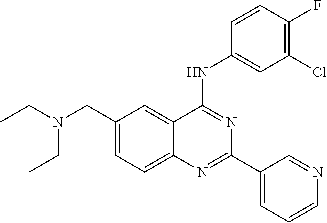 [SMILES: CCN(CC)Cc1ccc2nc(-c3cccnc3)nc(Nc3ccc(F)c(Cl)c3)c2c1] |
| 791 | 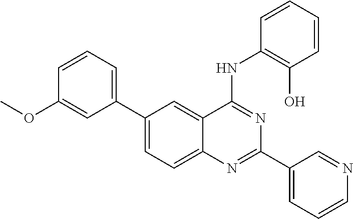 [SMILES: COc1cccc(-c2ccc3nc(-c4cccnc4)nc(Nc4ccccc4O)c3c2)c1] |
| 792 | 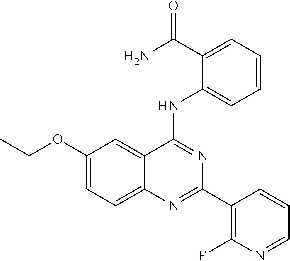 [SMILES: CCOc1ccc2nc(-c3cccnc3F)nc(Nc3ccccc3C(N)=O)c2c1] |
| 793 | 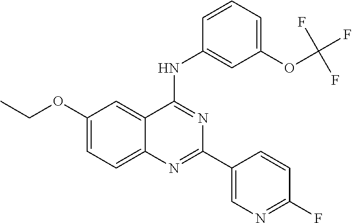 [SMILES: CCOc1ccc2nc(-c3ccc(F)nc3)nc(Nc3cccc(OC(F)(F)F)c3)c2c1] |
| 794 | 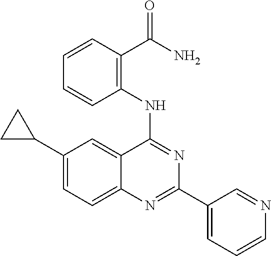 [SMILES: NC(=O)c1ccccc1Nc1nc(-c2cccnc2)nc2ccc(C3CC3)cc12] |
| 795 | 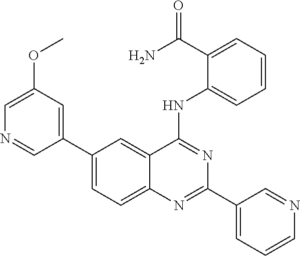 [SMILES: COc1cncc(-c2ccc3nc(-c4cccnc4)nc(Nc4ccccc4C(N)=O)c3c2)c1] |
| 796 | 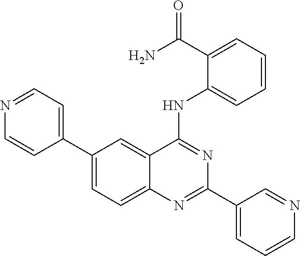 [SMILES: NC(=O)c1ccccc1Nc1nc(-c2cccnc2)nc2ccc(-c3ccncc3)cc12] |
| 797 | 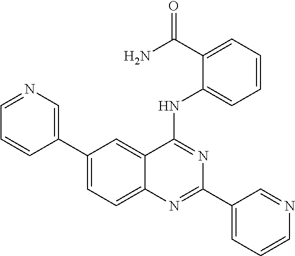 [SMILES: NC(=O)c1ccccc1Nc1nc(-c2cccnc2)nc2ccc(-c3cccnc3)cc12] |
| 798 | 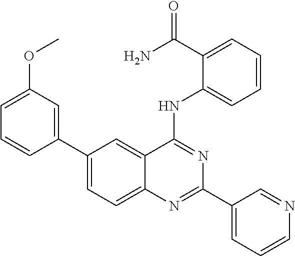 [SMILES: COc1cccc(-c2ccc3nc(-c4cccnc4)nc(Nc4ccccc4C(N)=O)c3c2)c1] |
| 799 | 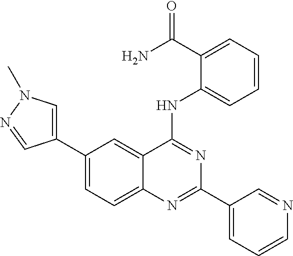 [SMILES: Cn1cc(-c2ccc3nc(-c4cccnc4)nc(Nc4ccccc4C(N)=O)c3c2)cn1] |
| 800 | 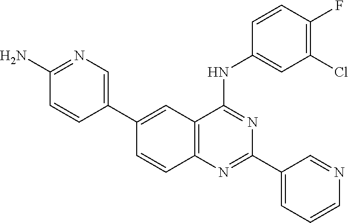 [SMILES: Nc1ccc(-c2ccc3nc(-c4cccnc4)nc(Nc4ccc(F)c(Cl)c4)c3c2)cn1] |
| 801 | 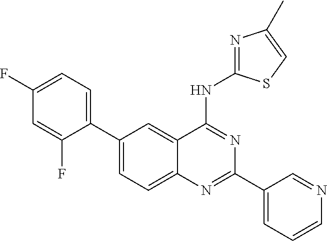 [SMILES: Cc1csc(Nc2nc(-c3cccnc3)nc3ccc(-c4ccc(F)cc4F)cc23)n1] |
| 802 | 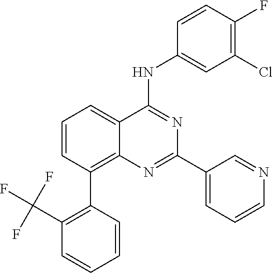 [SMILES: Fc1ccc(Nc2nc(-c3cccnc3)nc3c(-c4ccccc4C(F)(F)F)cccc23)cc1Cl] |
| 803 | 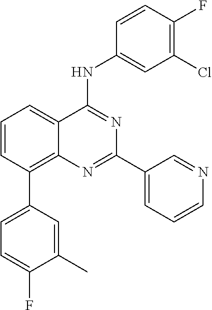 [SMILES: Cc1cc(-c2cccc3c(Nc4ccc(F)c(Cl)c4)nc(-c4cccnc4)nc23)ccc1F] |
| 804 | 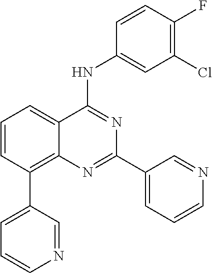 [SMILES: Fc1ccc(Nc2nc(-c3cccnc3)nc3c(-c4cccnc4)cccc23)cc1Cl] |
| 805 | 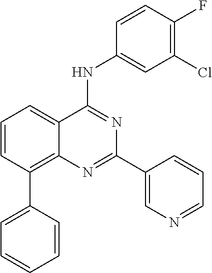 [SMILES: Fc1ccc(Nc2nc(-c3cccnc3)nc3c(-c4ccccc4)cccc23)cc1Cl] |
| 806 | 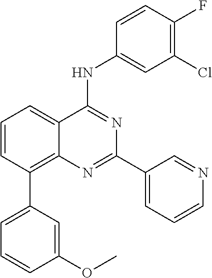 [SMILES: COc1cccc(-c2cccc3c(Nc4ccc(F)c(Cl)c4)nc(-c4cccnc4)nc23)c1] |
| 807 | 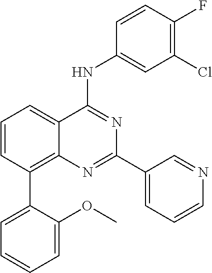 [SMILES: COc1ccccc1-c1cccc2c(Nc3ccc(F)c(Cl)c3)nc(-c3cccnc3)nc12] |
| 808 | 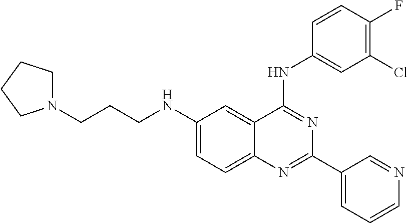 [SMILES: Fc1ccc(Nc2nc(-c3cccnc3)nc3ccc(NCCCN4CCCC4)cc23)cc1Cl] |
| 809 | 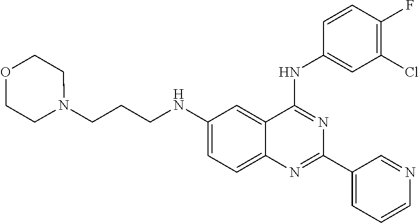 [SMILES: Fc1ccc(Nc2nc(-c3cccnc3)nc3ccc(NCCCN4CCOCC4)cc23)cc1Cl] |
| 810 | 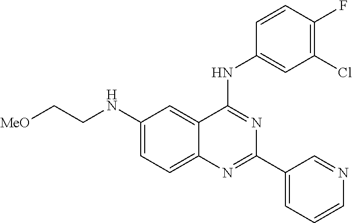 [SMILES: COCCNc1ccc2nc(-c3cccnc3)nc(Nc3ccc(F)c(Cl)c3)c2c1] |
| 811 | 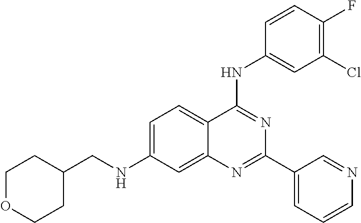 [SMILES: Fc1ccc(Nc2nc(-c3cccnc3)nc3cc(NCC4CCOCC4)ccc23)cc1Cl] |
| 812 | 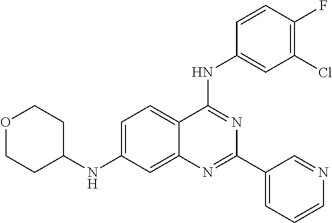 [SMILES: Fc1ccc(Nc2nc(-c3cccnc3)nc3cc(NC4CCOCC4)ccc23)cc1Cl] |
| 813 | 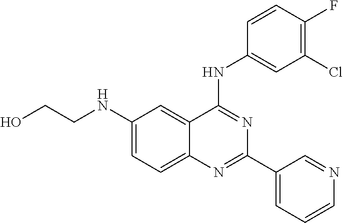 [SMILES: OCCNc1ccc2nc(-c3cccnc3)nc(Nc3ccc(F)c(Cl)c3)c2c1] |
| 814 | 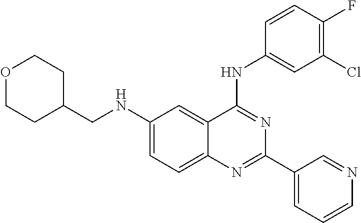 [SMILES: Fc1ccc(Nc2nc(-c3cccnc3)nc3ccc(NCC4CCOCC4)cc23)cc1Cl] |
| 815 | 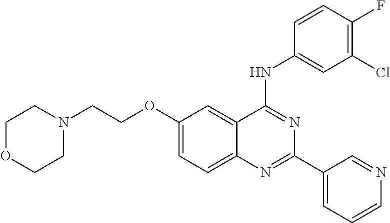 [SMILES: Fc1ccc(Nc2nc(-c3cccnc3)nc3ccc(OCCN4CCOCC4)cc23)cc1Cl] |
| 816 | 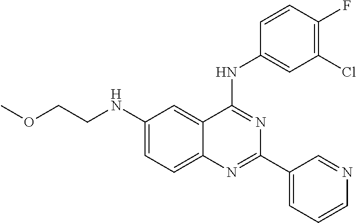 [SMILES: COCCNc1ccc2nc(-c3cccnc3)nc(Nc3ccc(F)c(Cl)c3)c2c1] |
| 817 | 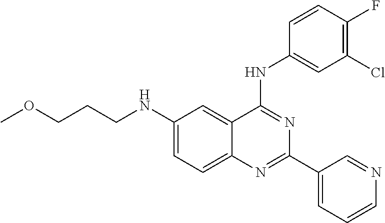 [SMILES: COCCCNc1ccc2nc(-c3cccnc3)nc(Nc3ccc(F)c(Cl)c3)c2c1] |
| 818 | 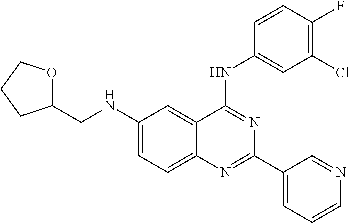 [SMILES: Fc1ccc(Nc2nc(-c3cccnc3)nc3ccc(NCC4CCCO4)cc23)cc1Cl] |
| 819 | 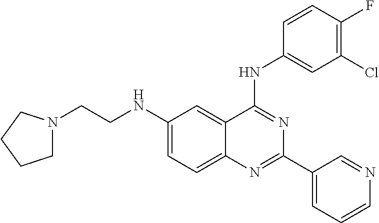 [SMILES: Fc1ccc(Nc2nc(-c3cccnc3)nc3ccc(NCCN4CCCC4)cc23)cc1Cl] |
| 820 | 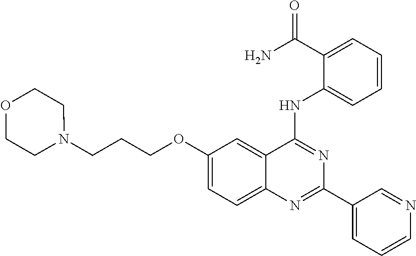 [SMILES: NC(=O)c1ccccc1Nc1nc(-c2cccnc2)nc2ccc(OCCCN3CCOCC3)cc12] |
| 821 | 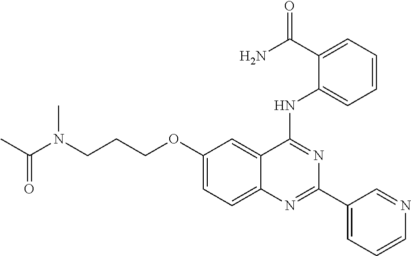 [SMILES: CC(=O)N(C)CCCOc1ccc2nc(-c3cccnc3)nc(Nc3ccccc3C(N)=O)c2c1] |
| 822 | 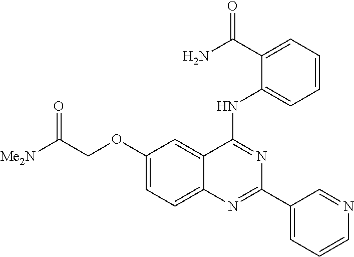 [SMILES: CN(C)C(=O)COc1ccc2nc(-c3cccnc3)nc(Nc3ccccc3C(N)=O)c2c1] |
| 823 | 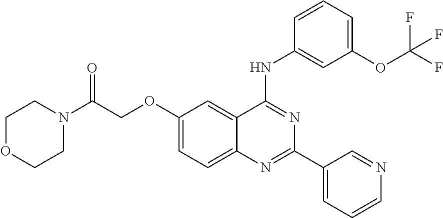 [SMILES: O=C(COc1ccc2nc(-c3cccnc3)nc(Nc3cccc(OC(F)(F)F)c3)c2c1)N1CCOCC1] |
| 824 | 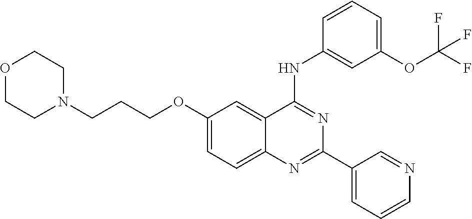 [SMILES: FC(F)(F)Oc1cccc(Nc2nc(-c3cccnc3)nc3ccc(OCCCN4CCOCC4)cc23)c1] |
| 825 | 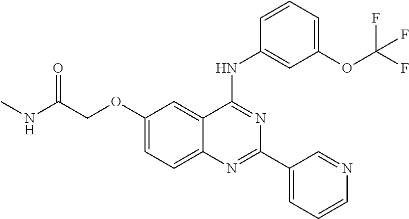 [SMILES: CNC(=O)COc1ccc2nc(-c3cccnc3)nc(Nc3cccc(OC(F)(F)F)c3)c2c1] |
| 826 | 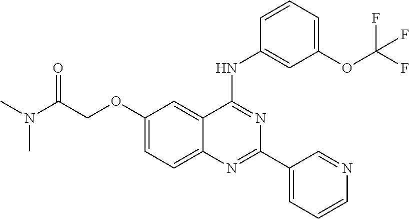 [SMILES: CN(C)C(=O)COc1ccc2nc(-c3cccnc3)nc(Nc3cccc(OC(F)(F)F)c3)c2c1] |
| 827 | 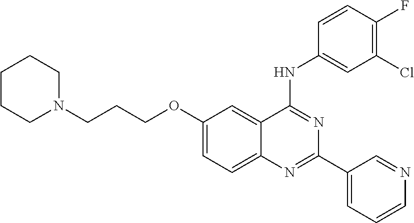 [SMILES: Fc1ccc(Nc2nc(-c3cccnc3)nc3ccc(OCCCN4CCCCC4)cc23)cc1Cl] |
| 828 | 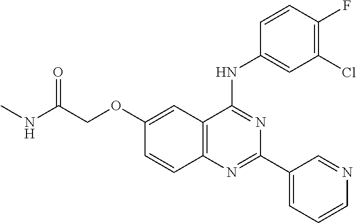 [SMILES: CNC(=O)COc1ccc2nc(-c3cccnc3)nc(Nc3ccc(F)c(Cl)c3)c2c1] |
| 829 | 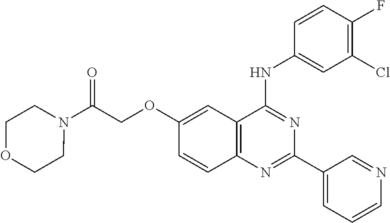 [SMILES: O=C(COc1ccc2nc(-c3cccnc3)nc(Nc3ccc(F)c(Cl)c3)c2c1)N1CCOCC1] |
| 830 | 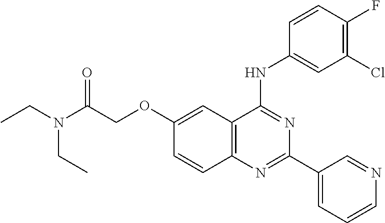 [SMILES: CCN(CC)C(=O)COc1ccc2nc(-c3cccnc3)nc(Nc3ccc(F)c(Cl)c3)c2c1] |
| 831 | 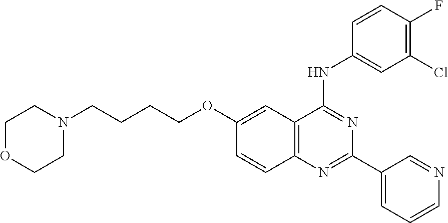 [SMILES: Fc1ccc(Nc2nc(-c3cccnc3)nc3ccc(OCCCCN4CCOCC4)cc23)cc1Cl] |
| 832 | 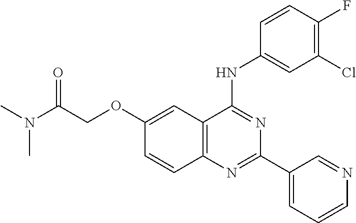 [SMILES: CN(C)C(=O)COc1ccc2nc(-c3cccnc3)nc(Nc3ccc(F)c(Cl)c3)c2c1] |
| 833 | 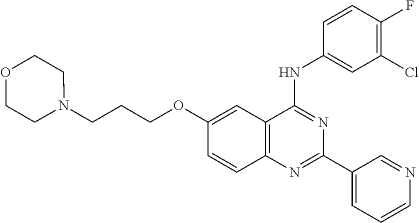 [SMILES: Fc1ccc(Nc2nc(-c3cccnc3)nc3ccc(OCCCN4CCOCC4)cc23)cc1Cl] |
| 834 | 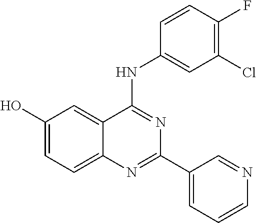 [SMILES: Oc1ccc2nc(-c3cccnc3)nc(Nc3ccc(F)c(Cl)c3)c2c1] |
| 835 | 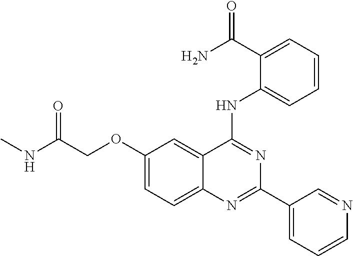 [SMILES: CNC(=O)COc1ccc2nc(-c3cccnc3)nc(Nc3ccccc3C(N)=O)c2c1] |
| 836 | 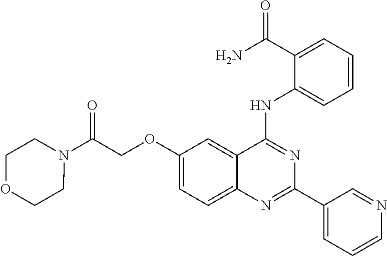 [SMILES: NC(=O)c1ccccc1Nc1nc(-c2cccnc2)nc2ccc(OCC(=O)N3CCOCC3)cc12] |
| 837 | 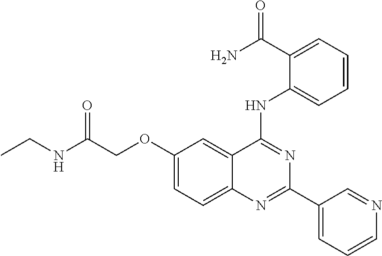 [SMILES: CCNC(=O)COc1ccc2nc(-c3cccnc3)nc(Nc3ccccc3C(N)=O)c2c1] |
| 838 | 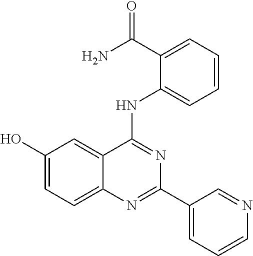 [SMILES: NC(=O)c1ccccc1Nc1nc(-c2cccnc2)nc2ccc(O)cc12] |
| 839 | 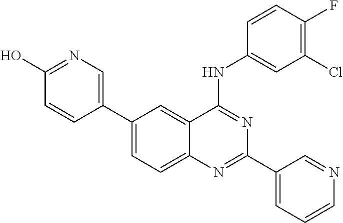 [SMILES: Oc1ccc(-c2ccc3nc(-c4cccnc4)nc(Nc4ccc(F)c(Cl)c4)c3c2)cn1] |
| 840 | 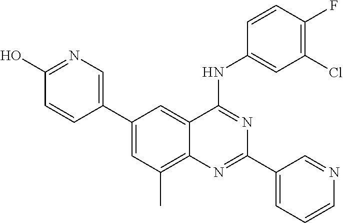 [SMILES: Cc1cc(-c2ccc(O)nc2)cc2c(Nc3ccc(F)c(Cl)c3)nc(-c3cccnc3)nc12] |
| 841 | 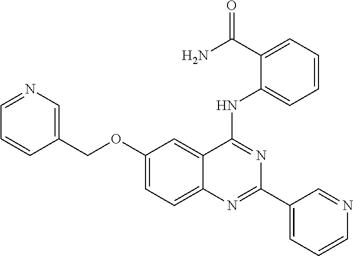 [SMILES: NC(=O)c1ccccc1Nc1nc(-c2cccnc2)nc2ccc(OCc3cccnc3)cc12] |
| 842 | 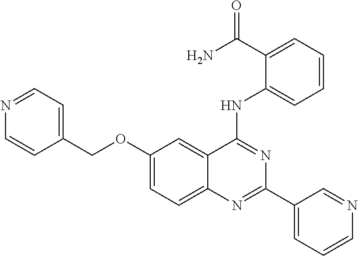 [SMILES: NC(=O)c1ccccc1Nc1nc(-c2cccnc2)nc2ccc(OCc3ccncc3)cc12] |
| 843 | 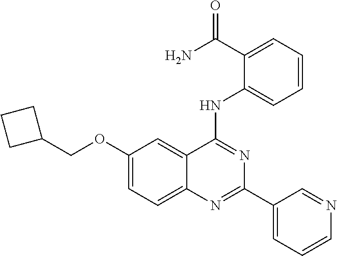 [SMILES: NC(=O)c1ccccc1Nc1nc(-c2cccnc2)nc2ccc(OCC3CCC3)cc12] |
| 844 | 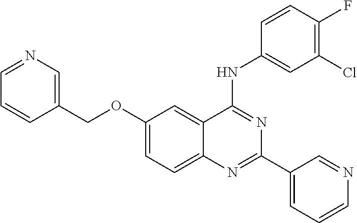 [SMILES: Fc1ccc(Nc2nc(-c3cccnc3)nc3ccc(OCc4cccnc4)cc23)cc1Cl] |
| 845 | 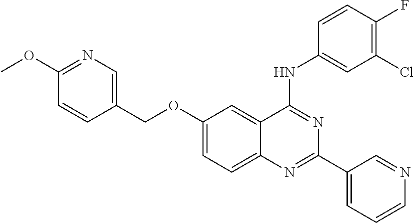 [SMILES: COc1ccc(COc2ccc3nc(-c4cccnc4)nc(Nc4ccc(F)c(Cl)c4)c3c2)cn1] |
| 846 | 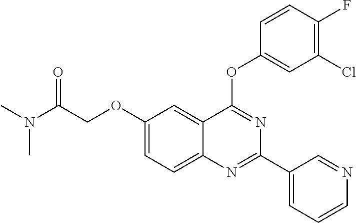 [SMILES: CN(C)C(=O)COc1ccc2nc(-c3cccnc3)nc(Oc3ccc(F)c(Cl)c3)c2c1] |
| 847 | 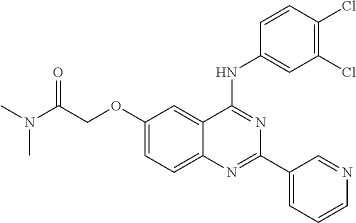 [SMILES: CN(C)C(=O)COc1ccc2nc(-c3cccnc3)nc(Nc3ccc(Cl)c(Cl)c3)c2c1] |
| 848 | 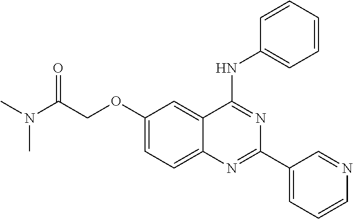 [SMILES: CN(C)C(=O)COc1ccc2nc(-c3cccnc3)nc(Nc3ccccc3)c2c1] |
| 849 | 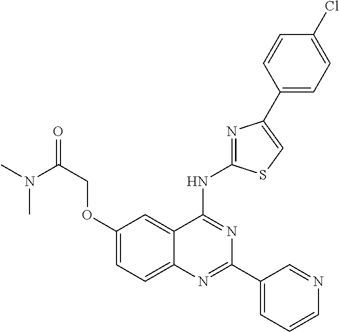 [SMILES: CN(C)C(=O)COc1ccc2nc(-c3cccnc3)nc(Nc3nc(-c4ccc(Cl)cc4)cs3)c2c1] |
| 850 | 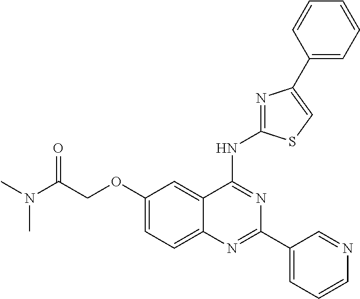 [SMILES: CN(C)C(=O)COc1ccc2nc(-c3cccnc3)nc(Nc3nc(-c4ccccc4)cs3)c2c1] |
| 851 | 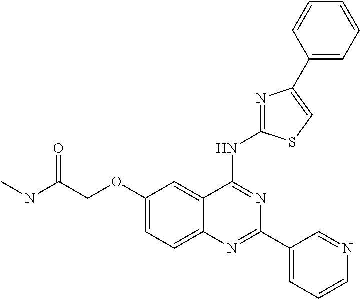 [SMILES: CNC(=O)COc1ccc2nc(-c3cccnc3)nc(Nc3nc(-c4ccccc4)cs3)c2c1] |
| 852 | 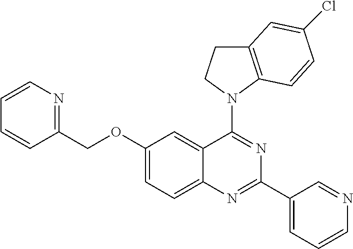 [SMILES: Clc1ccc2c(c1)CCN2c1nc(-c2cccnc2)nc2ccc(OCc3ccccn3)cc12] |
| 853 | 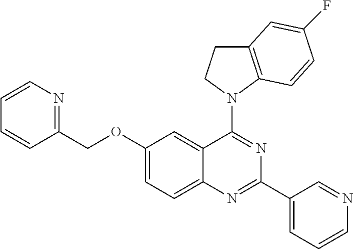 [SMILES: Fc1ccc2c(c1)CCN2c1nc(-c2cccnc2)nc2ccc(OCc3ccccn3)cc12] |
| 854 | 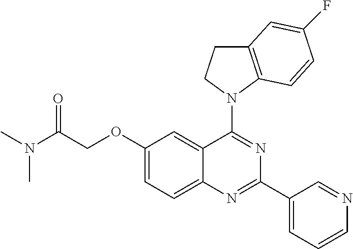 [SMILES: CN(C)C(=O)COc1ccc2nc(-c3cccnc3)nc(N3CCc4cc(F)ccc43)c2c1] |
| 855 | 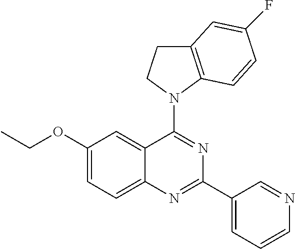 [SMILES: CCOc1ccc2nc(-c3cccnc3)nc(N3CCc4cc(F)ccc43)c2c1] |
| 856 | 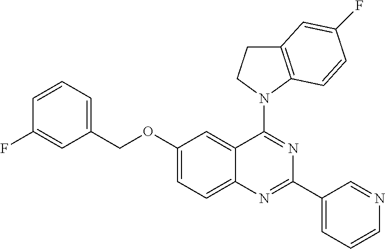 [SMILES: Fc1cccc(COc2ccc3nc(-c4cccnc4)nc(N4CCc5cc(F)ccc54)c3c2)c1] |
| 857 | 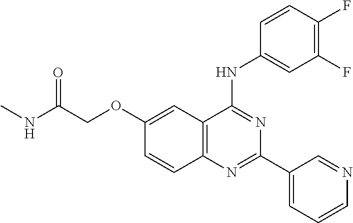 [SMILES: CNC(=O)COc1ccc2nc(-c3cccnc3)nc(Nc3ccc(F)c(F)c3)c2c1] |
| 858 | 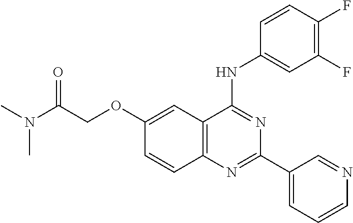 [SMILES: CN(C)C(=O)COc1ccc2nc(-c3cccnc3)nc(Nc3ccc(F)c(F)c3)c2c1] |
| 859 | 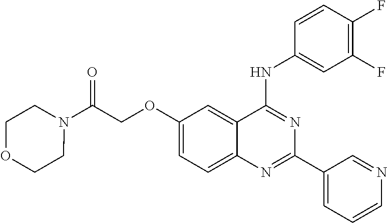 [SMILES: O=C(COc1ccc2nc(-c3cccnc3)nc(Nc3ccc(F)c(F)c3)c2c1)N1CCOCC1] |
| 860 | 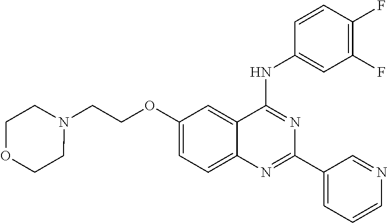 [SMILES: Fc1ccc(Nc2nc(-c3cccnc3)nc3ccc(OCCN4CCOCC4)cc23)cc1F] |
| 861 | 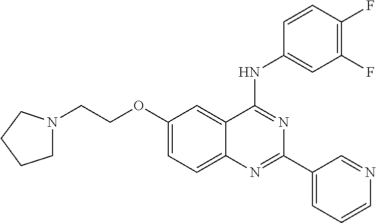 [SMILES: Fc1ccc(Nc2nc(-c3cccnc3)nc3ccc(OCCN4CCCC4)cc23)cc1F] |
| 862 | 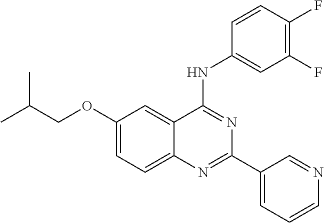 [SMILES: CC(C)COc1ccc2nc(-c3cccnc3)nc(Nc3ccc(F)c(F)c3)c2c1] |
| 863 | 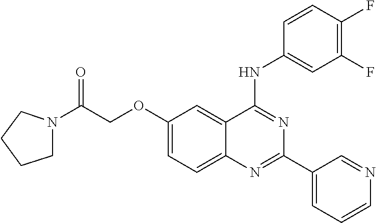 [SMILES: O=C(COc1ccc2nc(-c3cccnc3)nc(Nc3ccc(F)c(F)c3)c2c1)N1CCCC1] |
| 864 | 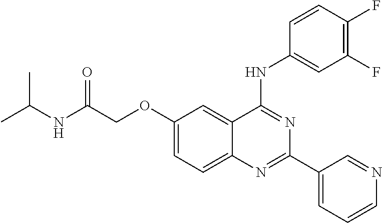 [SMILES: CC(C)NC(=O)COc1ccc2nc(-c3cccnc3)nc(Nc3ccc(F)c(F)c3)c2c1] |
| 865 | 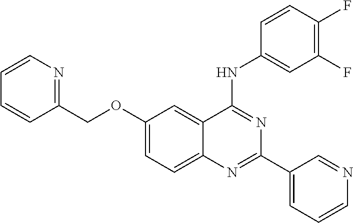 [SMILES: Fc1ccc(Nc2nc(-c3cccnc3)nc3ccc(OCc4ccccn4)cc23)cc1F] |
| 866 | 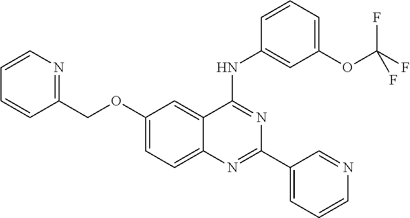 [SMILES: FC(F)(F)Oc1cccc(Nc2nc(-c3cccnc3)nc3ccc(OCc4ccccn4)cc23)c1] |
| 867 | 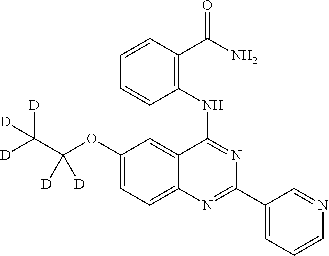 [SMILES: [2H]C([2H])([2H])C([2H])([2H])Oc1ccc2nc(-c3cccnc3)nc(Nc3ccccc3C(N)=O)c2c1] |
| 868 | 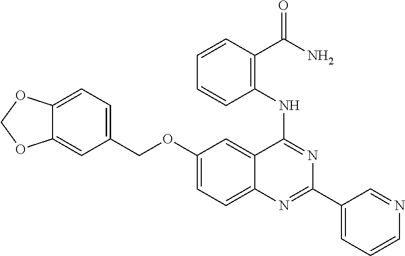 [SMILES: NC(=O)c1ccccc1Nc1nc(-c2cccnc2)nc2ccc(OCc3ccc4c(c3)OCO4)cc12] |
| 869 | 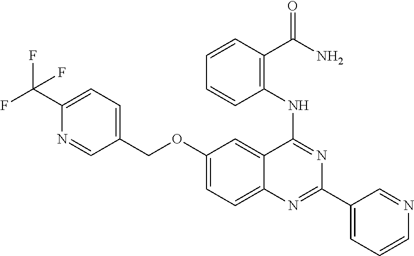 [SMILES: NC(=O)c1ccccc1Nc1nc(-c2cccnc2)nc2ccc(OCc3ccc(C(F)(F)F)nc3)cc12] |
| 870 | 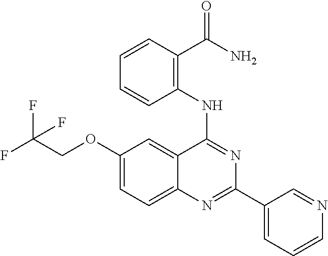 [SMILES: NC(=O)c1ccccc1Nc1nc(-c2cccnc2)nc2ccc(OCC(F)(F)F)cc12] |
| 871 | 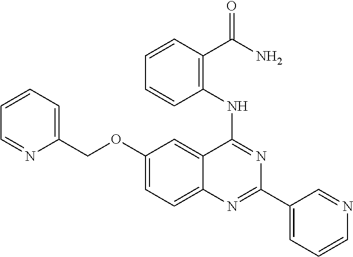 [SMILES: NC(=O)c1ccccc1Nc1nc(-c2cccnc2)nc2ccc(OCc3ccccn3)cc12] |
| 872 | 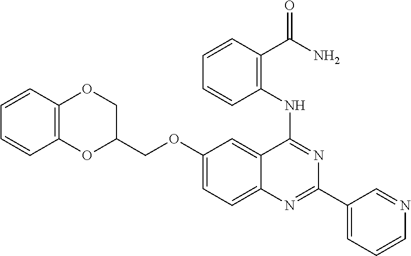 [SMILES: NC(=O)c1ccccc1Nc1nc(-c2cccnc2)nc2ccc(OCC3COc4ccccc4O3)cc12] |
| 873 | 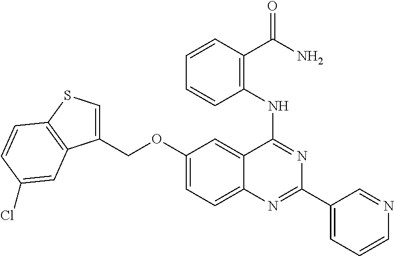 [SMILES: NC(=O)c1ccccc1Nc1nc(-c2cccnc2)nc2ccc(OCc3csc4ccc(Cl)cc34)cc12] |
| 874 | 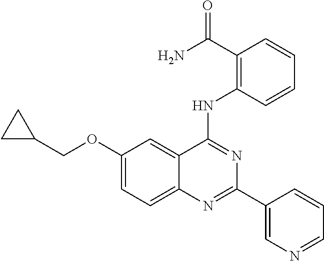 [SMILES: NC(=O)c1ccccc1Nc1nc(-c2cccnc2)nc2ccc(OCC3CC3)cc12] |
| 875 | 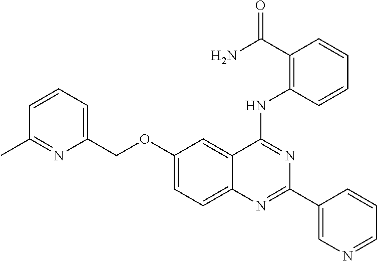 [SMILES: Cc1cccc(COc2ccc3nc(-c4cccnc4)nc(Nc4ccccc4C(N)=O)c3c2)n1] |
| 876 | 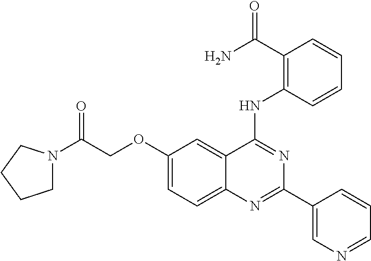 [SMILES: NC(=O)c1ccccc1Nc1nc(-c2cccnc2)nc2ccc(OCC(=O)N3CCCC3)cc12] |
| 1757 | 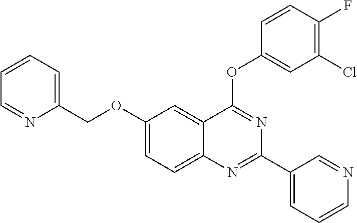 [SMILES: Fc1ccc(Oc2nc(-c3cccnc3)nc3ccc(OCc4ccccn4)cc23)cc1Cl] |
| 877 | 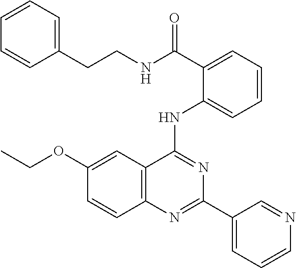 [SMILES: CCOc1ccc2nc(-c3cccnc3)nc(Nc3ccccc3C(=O)NCCc3ccccc3)c2c1] |
| 884 | 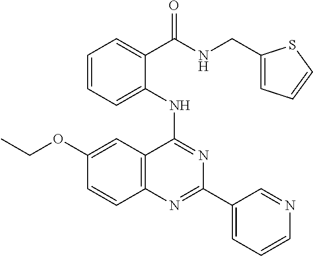 [SMILES: CCOc1ccc2nc(-c3cccnc3)nc(Nc3ccccc3C(=O)NCc3cccs3)c2c1] |
| 878 | 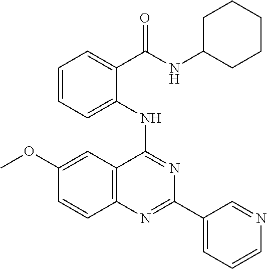 [SMILES: COc1ccc2nc(-c3cccnc3)nc(Nc3ccccc3C(=O)NC3CCCCC3)c2c1] |
| 879 | 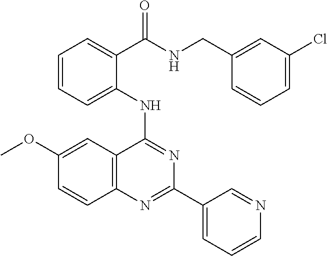 [SMILES: COc1ccc2nc(-c3cccnc3)nc(Nc3ccccc3C(=O)NCc3cccc(Cl)c3)c2c1] |
| 880 | 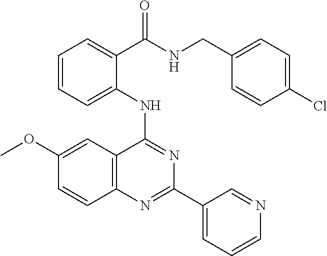 [SMILES: COc1ccc2nc(-c3cccnc3)nc(Nc3ccccc3C(=O)NCc3ccc(Cl)cc3)c2c1] |
| 881 | 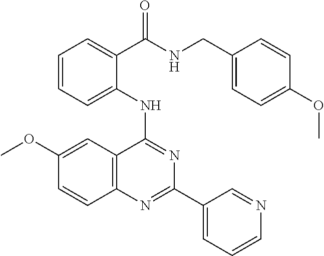 |
| 882 | 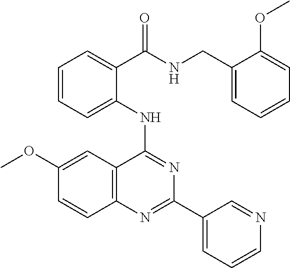 |
| 883 | 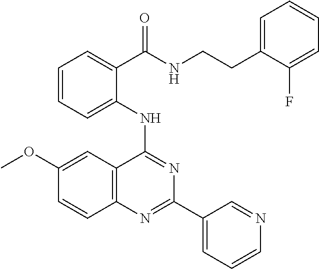 [SMILES: COc1ccc2nc(-c3cccnc3)nc(Nc3ccccc3C(=O)NCCc3ccccc3F)c2c1] |
| 891 | 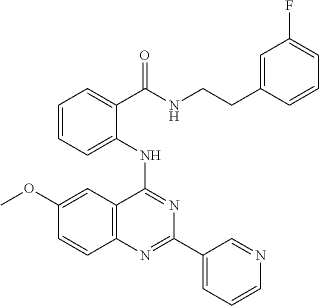 [SMILES: COc1ccc2nc(-c3cccnc3)nc(Nc3ccccc3C(=O)NCCc3cccc(F)c3)c2c1] |
| 885 | 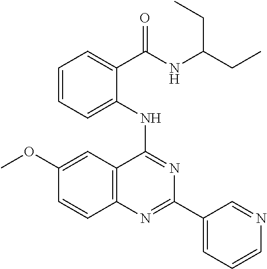 [SMILES: CCC(CC)NC(=O)c1ccccc1Nc1nc(-c2cccnc2)nc2ccc(OC)cc12] |
| 886 | 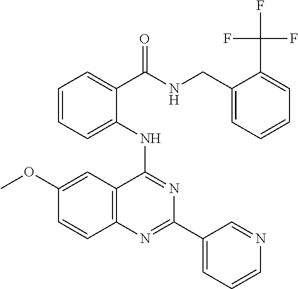 [SMILES: COc1ccc2nc(-c3cccnc3)nc(Nc3ccccc3C(=O)NCc3ccccc3C(F)(F)F)c2c1] |
| 887 | 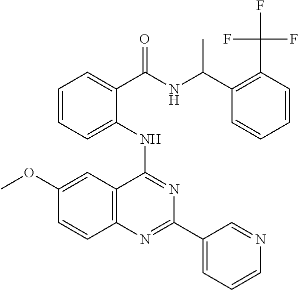 [SMILES: COc1ccc2nc(-c3cccnc3)nc(Nc3ccccc3C(=O)NC(C)c3ccccc3C(F)(F)F)c2c1] |
| 888 | 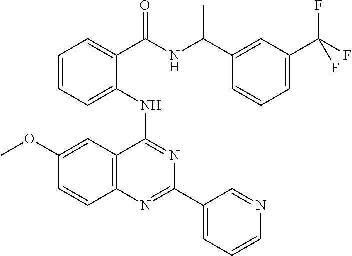 [SMILES: COc1ccc2nc(-c3cccnc3)nc(Nc3ccccc3C(=O)NC(C)c3cccc(C(F)(F)F)c3)c2c1] |
| 889 | 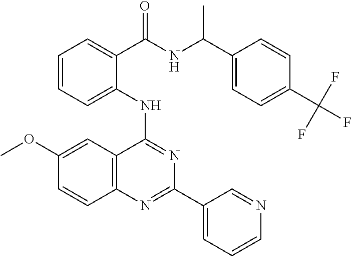 [SMILES: COc1ccc2nc(-c3cccnc3)nc(Nc3ccccc3C(=O)NC(C)c3ccc(C(F)(F)F)cc3)c2c1] |
| 890 | 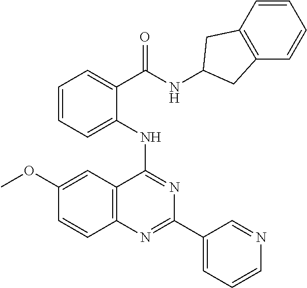 [SMILES: COc1ccc2nc(-c3cccnc3)nc(Nc3ccccc3C(=O)NC3Cc4ccccc4C3)c2c1] |
| 898 | 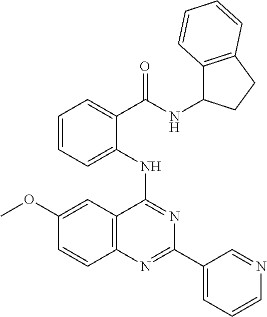 [SMILES: COc1ccc2nc(-c3cccnc3)nc(Nc3ccccc3C(=O)NC3CCc4ccccc43)c2c1] |
| 891 | 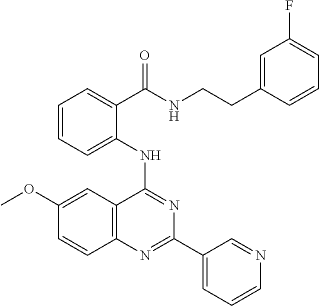 [SMILES: COc1ccc2nc(-c3cccnc3)nc(Nc3ccccc3C(=O)NCCc3cccc(F)c3)c2c1] |
| 892 | 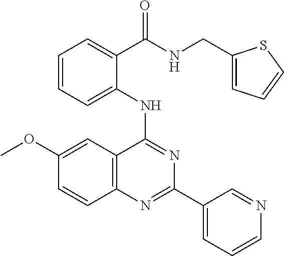 [SMILES: COc1ccc2nc(-c3cccnc3)nc(Nc3ccccc3C(=O)NCc3cccs3)c2c1] |
| 893 | 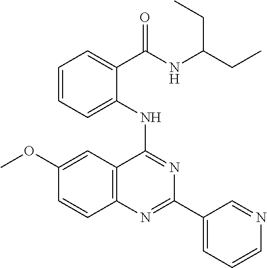 [SMILES: CCC(CC)NC(=O)c1ccccc1Nc1nc(-c2cccnc2)nc2ccc(OC)cc12] |
| 894 | 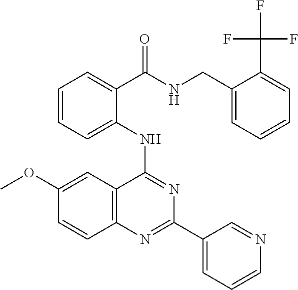 [SMILES: COc1ccc2nc(-c3cccnc3)nc(Nc3ccccc3C(=O)NCc3ccccc3C(F)(F)F)c2c1] |
| 895 | 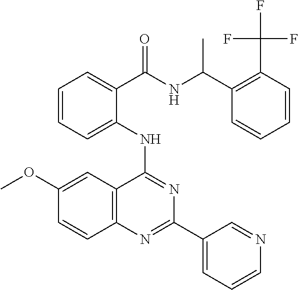 [SMILES: COc1ccc2nc(-c3cccnc3)nc(Nc3ccccc3C(=O)NC(C)c3ccccc3C(F)(F)F)c2c1] |
| 896 | 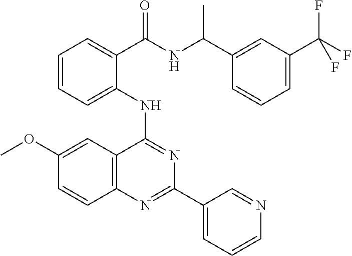 [SMILES: COc1ccc2nc(-c3cccnc3)nc(Nc3ccccc3C(=O)NC(C)c3cccc(C(F)(F)F)c3)c2c1] |
| 897 | 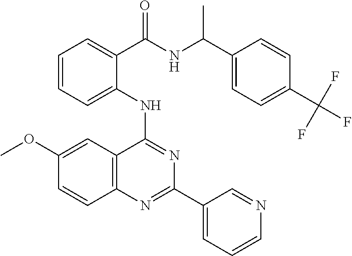 [SMILES: COc1ccc2nc(-c3cccnc3)nc(Nc3ccccc3C(=O)NC(C)c3ccc(C(F)(F)F)cc3)c2c1] |
| 898 | 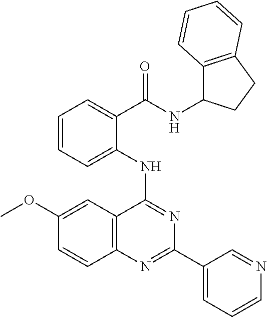 [SMILES: COc1ccc2nc(-c3cccnc3)nc(Nc3ccccc3C(=O)NC3CCc4ccccc43)c2c1] |
| 899 | 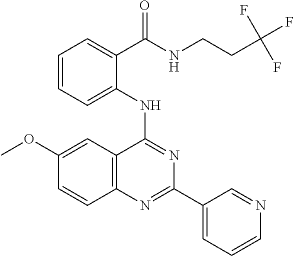 [SMILES: COc1ccc2nc(-c3cccnc3)nc(Nc3ccccc3C(=O)NCCC(F)(F)F)c2c1] |
| 900 | 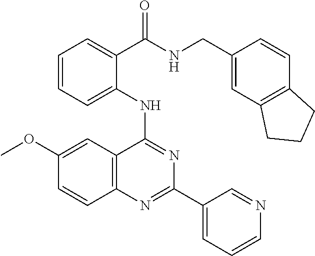 [SMILES: COc1ccc2nc(-c3cccnc3)nc(Nc3ccccc3C(=O)NCc3ccc4c(c3)CCC4)c2c1] |
| 901 | 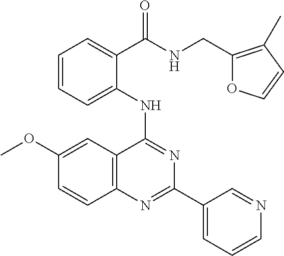 [SMILES: COc1ccc2nc(-c3cccnc3)nc(Nc3ccccc3C(=O)NCc3occc3C)c2c1] |
| 902 | 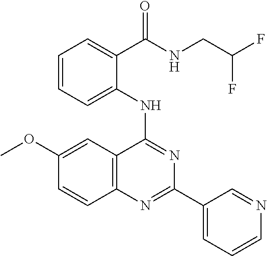 [SMILES: COc1ccc2nc(-c3cccnc3)nc(Nc3ccccc3C(=O)NCC(F)F)c2c1] |
| 903 | 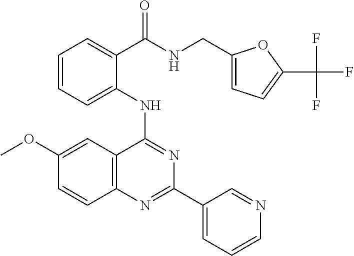 [SMILES: COc1ccc2nc(-c3cccnc3)nc(Nc3ccccc3C(=O)NCc3ccc(C(F)(F)F)o3)c2c1] |
| 904 | 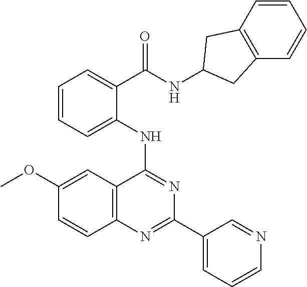 [SMILES: COc1ccc2nc(-c3cccnc3)nc(Nc3ccccc3C(=O)NC3Cc4ccccc4C3)c2c1] |
| 905 | 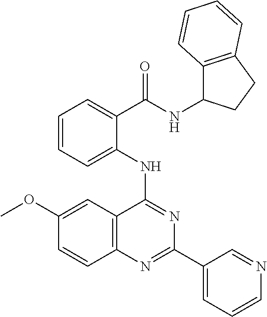 [SMILES: COc1ccc2nc(-c3cccnc3)nc(Nc3ccccc3C(=O)NC3CCc4ccccc43)c2c1] |
| 906 | 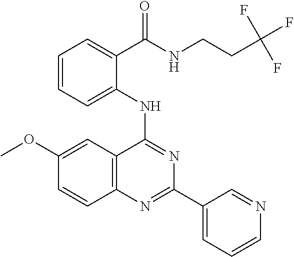 [SMILES: COc1ccc2nc(-c3cccnc3)nc(Nc3ccccc3C(=O)NCCC(F)(F)F)c2c1] |
| 907 | 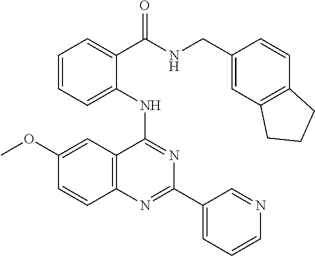 [SMILES: COc1ccc2nc(-c3cccnc3)nc(Nc3ccccc3C(=O)NCc3ccc4c(c3)CCC4)c2c1] |
| 908 | 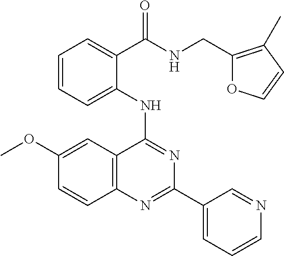 [SMILES: COc1ccc2nc(-c3cccnc3)nc(Nc3ccccc3C(=O)NCc3occc3C)c2c1] |
| 913 | 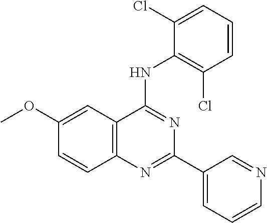 [SMILES: COc1ccc2nc(-c3cccnc3)nc(Nc3c(Cl)cccc3Cl)c2c1] |
| 915 | 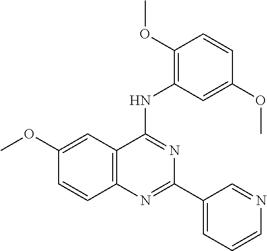 |
| 917 | 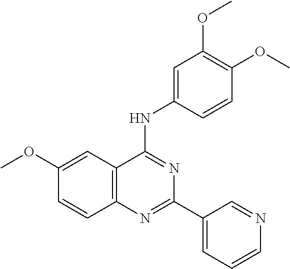 |
| 909 | 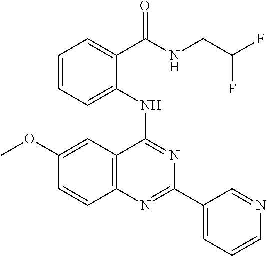 [SMILES: COc1ccc2nc(-c3cccnc3)nc(Nc3ccccc3C(=O)NCC(F)F)c2c1] |
| 910 | 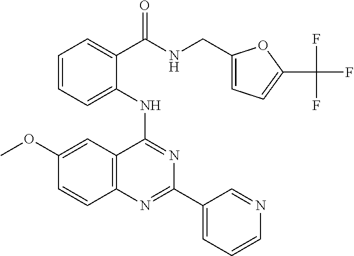 [SMILES: COc1ccc2nc(-c3cccnc3)nc(Nc3ccccc3C(=O)NCc3ccc(C(F)(F)F)o3)c2c1] |
| 911 | 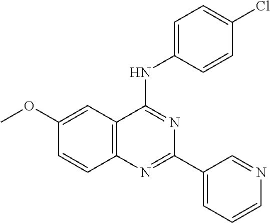 [SMILES: COc1ccc2nc(-c3cccnc3)nc(Nc3ccc(Cl)cc3)c2c1] |
| 912 | 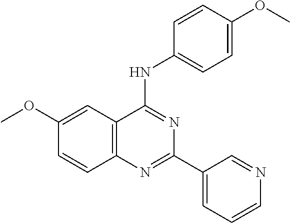 |
| 914 | 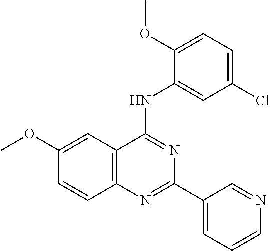 [SMILES: COc1ccc2nc(-c3cccnc3)nc(Nc3cc(Cl)ccc3OC)c2c1] |
| 916 | 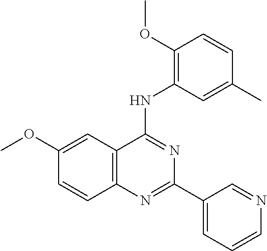 |
| 918 | 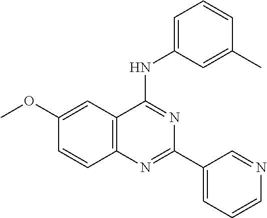 |
| 919 | 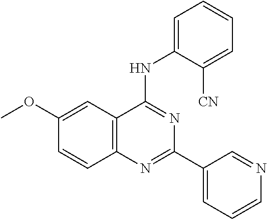 [SMILES: COc1ccc2nc(-c3cccnc3)nc(Nc3ccccc3C#N)c2c1] |
| 920 | 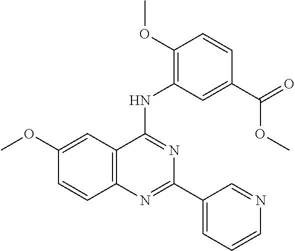 |
| 921 | 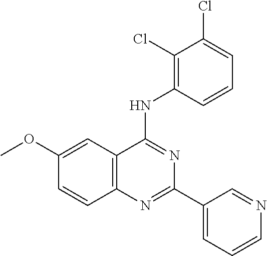 [SMILES: COc1ccc2nc(-c3cccnc3)nc(Nc3cccc(Cl)c3Cl)c2c1] |
| 922 | 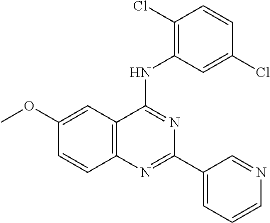 [SMILES: COc1ccc2nc(-c3cccnc3)nc(Nc3cc(Cl)ccc3Cl)c2c1] |
| 923 | 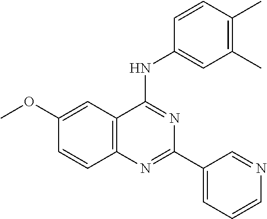 |
| 924 | 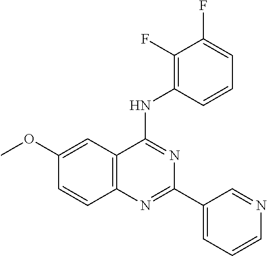 [SMILES: COc1ccc2nc(-c3cccnc3)nc(Nc3cccc(F)c3F)c2c1] |
| 925 | 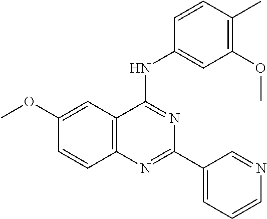 |
| 926 | 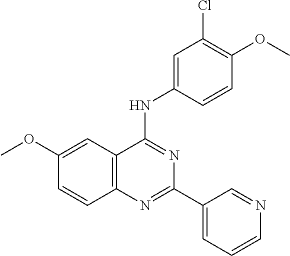 |
| 927 | 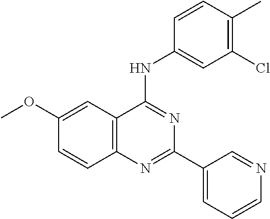 [SMILES: COc1ccc2nc(-c3cccnc3)nc(Nc3ccc(C)c(Cl)c3)c2c1] |
| 928 | 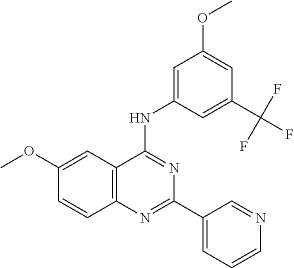 [SMILES: COc1cc(Nc2nc(-c3cccnc3)nc3ccc(OC)cc23)cc(C(F)(F)F)c1] |
| 929 | 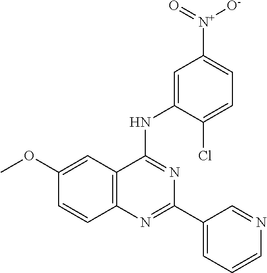 [SMILES: COc1ccc2nc(-c3cccnc3)nc(Nc3cc([N+](=O)[O-])ccc3Cl)c2c1] |
| 930 | 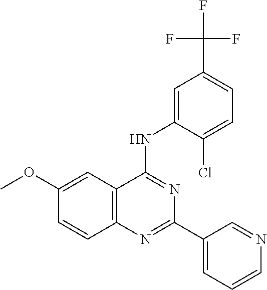 [SMILES: COc1ccc2nc(-c3cccnc3)nc(Nc3cc(C(F)(F)F)ccc3Cl)c2c1] |
| 931 | 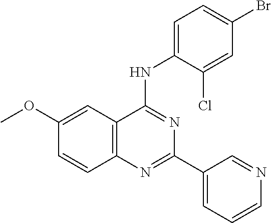 [SMILES: COc1ccc2nc(-c3cccnc3)nc(Nc3ccc(Br)cc3Cl)c2c1] |
| 932 | 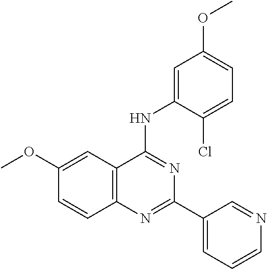 [SMILES: COc1ccc(Cl)c(Nc2nc(-c3cccnc3)nc3ccc(OC)cc23)c1] |
| 933 | 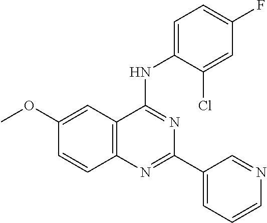 [SMILES: COc1ccc2nc(-c3cccnc3)nc(Nc3ccc(F)cc3Cl)c2c1] |
| 934 | 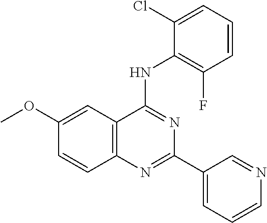 [SMILES: COc1ccc2nc(-c3cccnc3)nc(Nc3c(F)cccc3Cl)c2c1] |
| 935 | 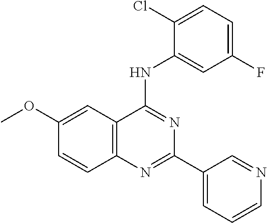 [SMILES: COc1ccc2nc(-c3cccnc3)nc(Nc3cc(F)ccc3Cl)c2c1] |
| 936 | 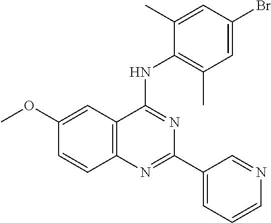 [SMILES: COc1ccc2nc(-c3cccnc3)nc(Nc3c(C)cc(Br)cc3C)c2c1] |
| 937 | 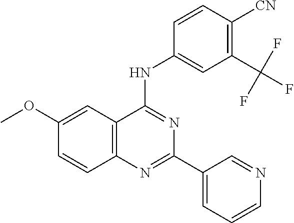 [SMILES: COc1ccc2nc(-c3cccnc3)nc(Nc3ccc(C#N)c(C(F)(F)F)c3)c2c1] |
| 938 | 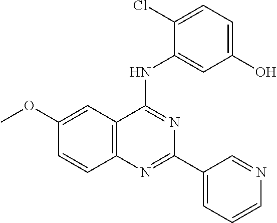 [SMILES: COc1ccc2nc(-c3cccnc3)nc(Nc3cc(O)ccc3Cl)c2c1] |
| 939 | 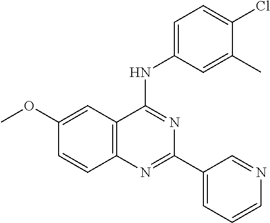 |
| 940 | 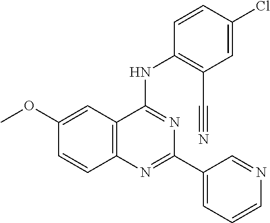 [SMILES: COc1ccc2nc(-c3cccnc3)nc(Nc3ccc(Cl)cc3C#N)c2c1] |
| 941 | 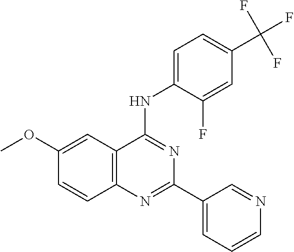 [SMILES: COc1ccc2nc(-c3cccnc3)nc(Nc3ccc(C(F)(F)F)cc3F)c2c1] |
| 942 | 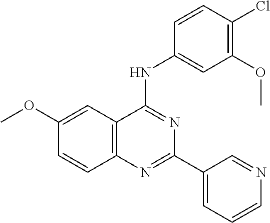 |
| 943 | 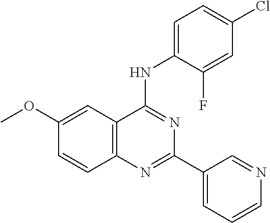 [SMILES: COc1ccc2nc(-c3cccnc3)nc(Nc3ccc(Cl)cc3F)c2c1] |
| 944 | 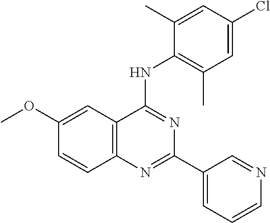 [SMILES: COc1ccc2nc(-c3cccnc3)nc(Nc3c(C)cc(Cl)cc3C)c2c1] |
| 945 | 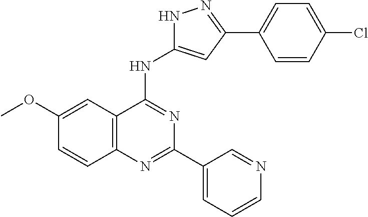 [SMILES: COc1ccc2nc(-c3cccnc3)nc(Nc3cc(-c4ccc(Cl)cc4)n[nH]3)c2c1] |
| 946 | 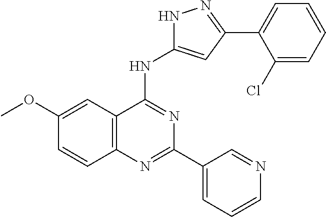 [SMILES: COc1ccc2nc(-c3cccnc3)nc(Nc3cc(-c4ccccc4Cl)n[nH]3)c2c1] |
| 947 | 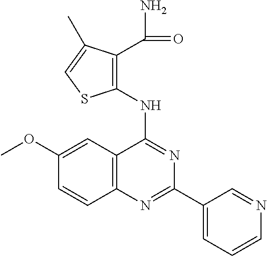 [SMILES: COc1ccc2nc(-c3cccnc3)nc(Nc3scc(C)c3C(N)=O)c2c1] |
| 948 | 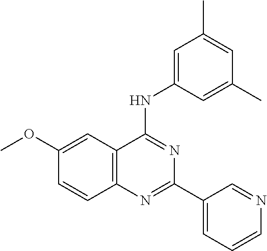 |
| 949 | 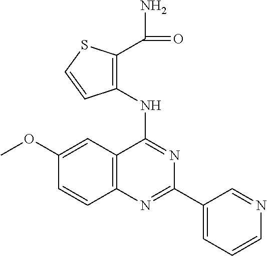 [SMILES: COc1ccc2nc(-c3cccnc3)nc(Nc3ccsc3C(N)=O)c2c1] |
| 950 | 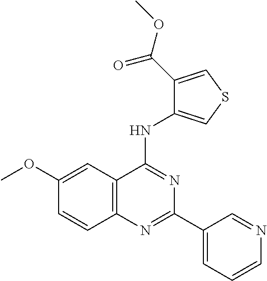 [SMILES: COC(=O)c1cscc1Nc1nc(-c2cccnc2)nc2ccc(OC)cc12] |
| 951 | 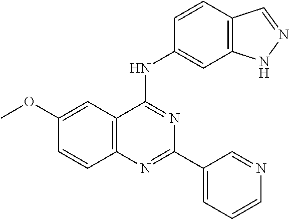 [SMILES: COc1ccc2nc(-c3cccnc3)nc(Nc3ccc4cn[nH]c4c3)c2c1] |
| 952 | 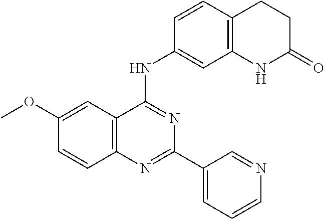 [SMILES: COc1ccc2nc(-c3cccnc3)nc(Nc3ccc4c(c3)NC(=O)CC4)c2c1] |
| 953 | 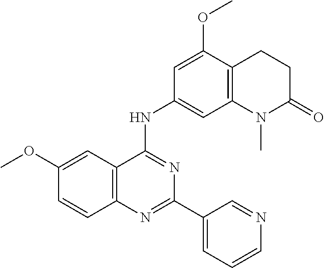 [SMILES: COc1ccc2nc(-c3cccnc3)nc(Nc3cc(OC)c4c(c3)N(C)C(=O)CC4)c2c1] |
| 954 | 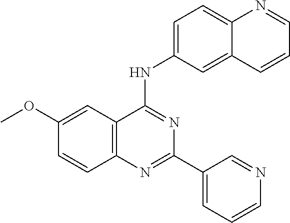 [SMILES: COc1ccc2nc(-c3cccnc3)nc(Nc3ccc4ncccc4c3)c2c1] |
| 955 | 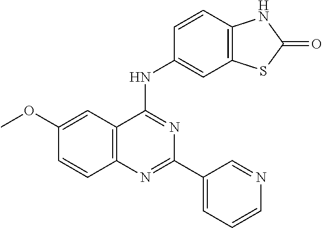 [SMILES: COc1ccc2nc(-c3cccnc3)nc(Nc3ccc4[nH]c(=O)sc4c3)c2c1] |
| 956 | 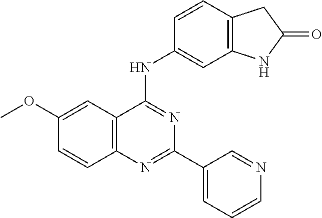 [SMILES: COc1ccc2nc(-c3cccnc3)nc(Nc3ccc4c(c3)NC(=O)C4)c2c1] |
| 957 | 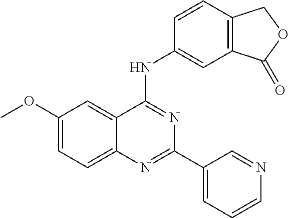 [SMILES: COc1ccc2nc(-c3cccnc3)nc(Nc3ccc4c(c3)C(=O)OC4)c2c1] |
| 958 | 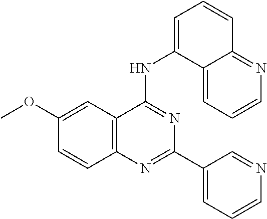 [SMILES: COc1ccc2nc(-c3cccnc3)nc(Nc3cccc4ncccc34)c2c1] |
| 959 | 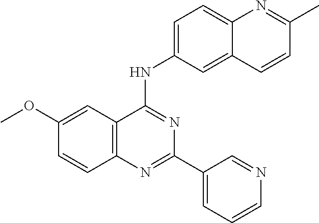 |
| 960 | 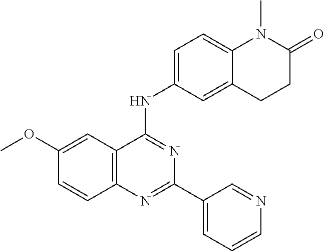 [SMILES: COc1ccc2nc(-c3cccnc3)nc(Nc3ccc4c(c3)CCC(=O)N4C)c2c1] |
| 961 | 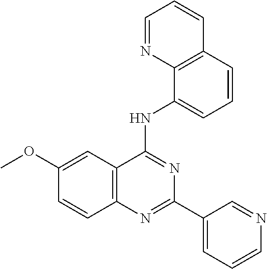 [SMILES: COc1ccc2nc(-c3cccnc3)nc(Nc3cccc4cccnc34)c2c1] |
| 962 | 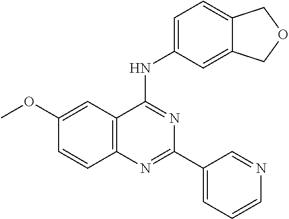 [SMILES: COc1ccc2nc(-c3cccnc3)nc(Nc3ccc4c(c3)COC4)c2c1] |
| 963 | 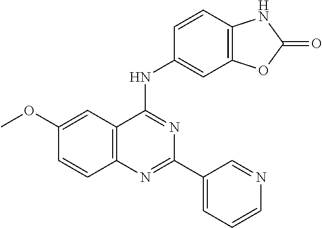 [SMILES: COc1ccc2nc(-c3cccnc3)nc(Nc3ccc4[nH]c(=O)oc4c3)c2c1] |
| 964 | 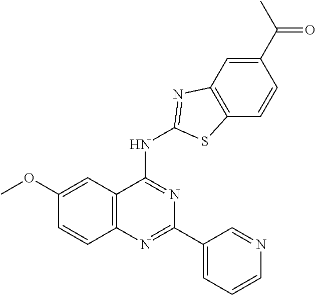 [SMILES: COc1ccc2nc(-c3cccnc3)nc(Nc3nc4cc(C(C)=O)ccc4s3)c2c1] |
| 965 | 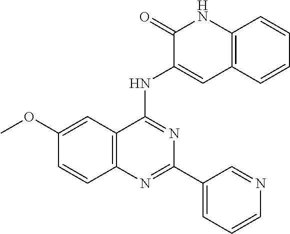 [SMILES: COc1ccc2nc(-c3cccnc3)nc(Nc3cc4ccccc4[nH]c3=O)c2c1] |
| 966 | 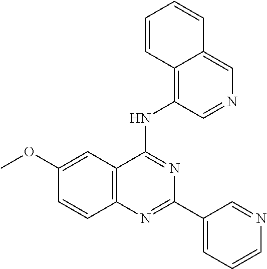 [SMILES: COc1ccc2nc(-c3cccnc3)nc(Nc3cncc4ccccc34)c2c1] |
| 967 | 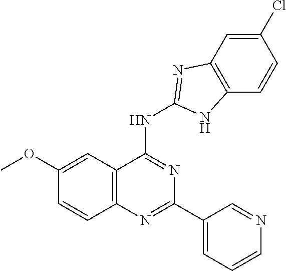 [SMILES: COc1ccc2nc(-c3cccnc3)nc(Nc3nc4cc(Cl)ccc4[nH]3)c2c1] |
| 969 | 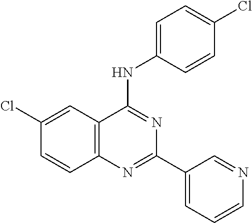 [SMILES: Clc1ccc(Nc2nc(-c3cccnc3)nc3ccc(Cl)cc23)cc1] |
| 970 | 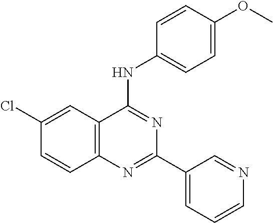 [SMILES: COc1ccc(Nc2nc(-c3cccnc3)nc3ccc(Cl)cc23)cc1] |
| 971 | 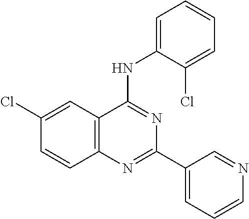 [SMILES: Clc1ccc2nc(-c3cccnc3)nc(Nc3ccccc3Cl)c2c1] |
| 972 | 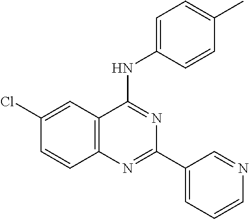 [SMILES: Cc1ccc(Nc2nc(-c3cccnc3)nc3ccc(Cl)cc23)cc1] |
| 973 | 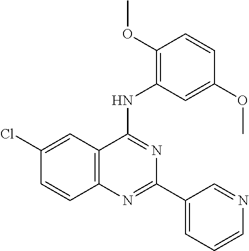 [SMILES: COc1ccc(OC)c(Nc2nc(-c3cccnc3)nc3ccc(Cl)cc23)c1] |
| 974 | 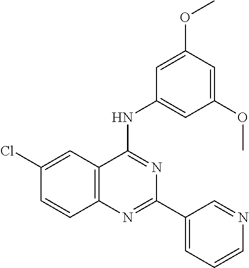 [SMILES: COc1cc(Nc2nc(-c3cccnc3)nc3ccc(Cl)cc23)cc(OC)c1] |
| 968 | 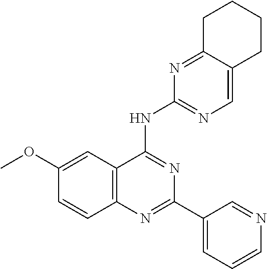 [SMILES: COc1ccc2nc(-c3cccnc3)nc(Nc3ncc4c(n3)CCCC4)c2c1] |
| 975 | 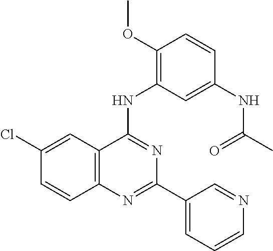 [SMILES: COc1ccc(NC(C)=O)cc1Nc1nc(-c2cccnc2)nc2ccc(Cl)cc12] |
| 976 | 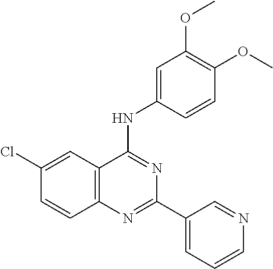 [SMILES: COc1ccc(Nc2nc(-c3cccnc3)nc3ccc(Cl)cc23)cc1OC] |
| 977 | 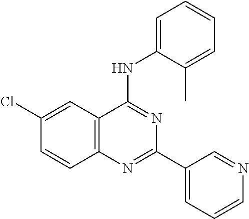 [SMILES: Cc1ccccc1Nc1nc(-c2cccnc2)nc2ccc(Cl)cc12] |
| 978 | 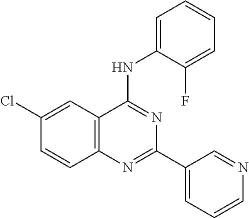 [SMILES: Fc1ccccc1Nc1nc(-c2cccnc2)nc2ccc(Cl)cc12] |
| 979 | 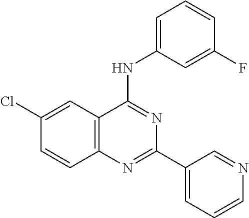 [SMILES: Fc1cccc(Nc2nc(-c3cccnc3)nc3ccc(Cl)cc23)c1] |
| 980 | 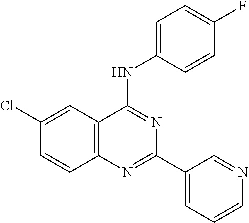 [SMILES: Fc1ccc(Nc2nc(-c3cccnc3)nc3ccc(Cl)cc23)cc1] |
| 981 | 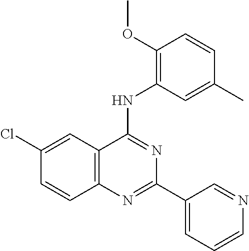 [SMILES: COc1ccc(C)cc1Nc1nc(-c2cccnc2)nc2ccc(Cl)cc12] |
| 982 | 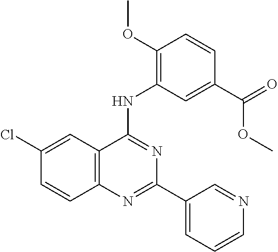 [SMILES: COC(=O)c1ccc(OC)c(Nc2nc(-c3cccnc3)nc3ccc(Cl)cc23)c1] |
| 983 | 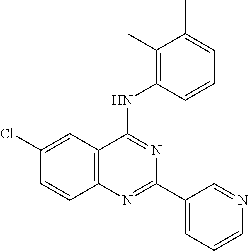 [SMILES: Cc1cccc(Nc2nc(-c3cccnc3)nc3ccc(Cl)cc23)c1C] |
| 984 | 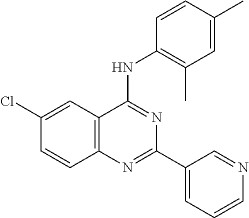 [SMILES: Cc1ccc(Nc2nc(-c3cccnc3)nc3ccc(Cl)cc23)c(C)c1] |
| 985 | 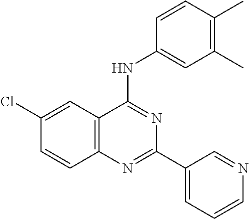 [SMILES: Cc1ccc(Nc2nc(-c3cccnc3)nc3ccc(Cl)cc23)cc1C] |
| 986 | 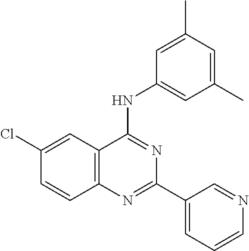 [SMILES: Cc1cc(C)cc(Nc2nc(-c3cccnc3)nc3ccc(Cl)cc23)c1] |
| 987 | 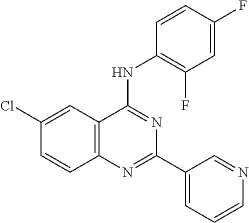 [SMILES: Fc1ccc(Nc2nc(-c3cccnc3)nc3ccc(Cl)cc23)c(F)c1] |
| 988 | 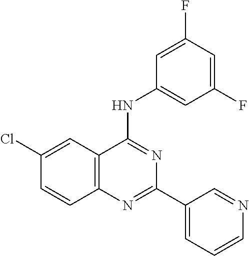 [SMILES: Fc1cc(F)cc(Nc2nc(-c3cccnc3)nc3ccc(Cl)cc23)c1] |
| 989 | 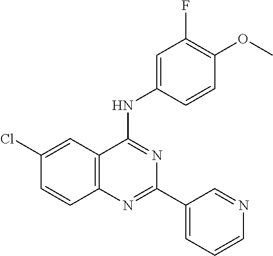 [SMILES: COc1ccc(Nc2nc(-c3cccnc3)nc3ccc(Cl)cc23)cc1F] |
| 990 | 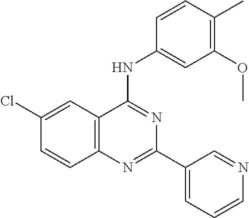 [SMILES: COc1cc(Nc2nc(-c3cccnc3)nc3ccc(Cl)cc23)ccc1C] |
| 991 | 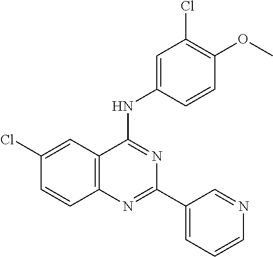 [SMILES: COc1ccc(Nc2nc(-c3cccnc3)nc3ccc(Cl)cc23)cc1Cl] |
| 992 | 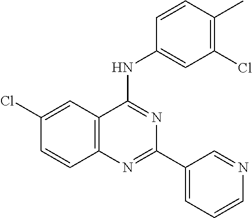 [SMILES: Cc1ccc(Nc2nc(-c3cccnc3)nc3ccc(Cl)cc23)cc1Cl] |
| 993 | 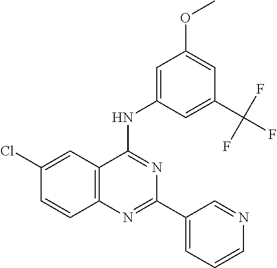 [SMILES: COc1cc(Nc2nc(-c3cccnc3)nc3ccc(Cl)cc23)cc(C(F)(F)F)c1] |
| 994 | 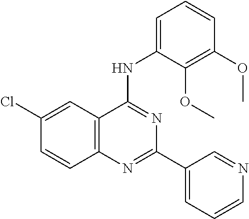 [SMILES: COc1cccc(Nc2nc(-c3cccnc3)nc3ccc(Cl)cc23)c1OC] |
| 995 | 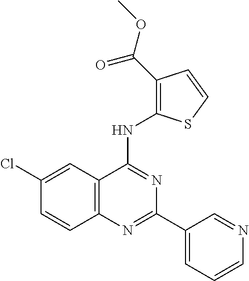 [SMILES: COC(=O)c1ccsc1Nc1nc(-c2cccnc2)nc2ccc(Cl)cc12] |
| 996 | 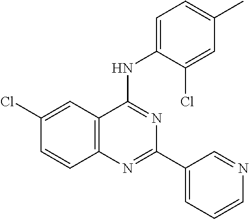 [SMILES: Cc1ccc(Nc2nc(-c3cccnc3)nc3ccc(Cl)cc23)c(Cl)c1] |
| 997 | 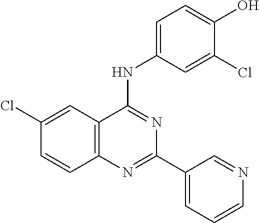 [SMILES: Oc1ccc(Nc2nc(-c3cccnc3)nc3ccc(Cl)cc23)cc1Cl] |
| 998 | 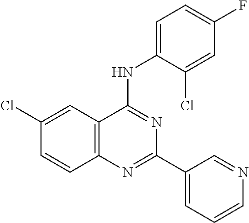 [SMILES: Fc1ccc(Nc2nc(-c3cccnc3)nc3ccc(Cl)cc23)c(Cl)c1] |
| 999 | 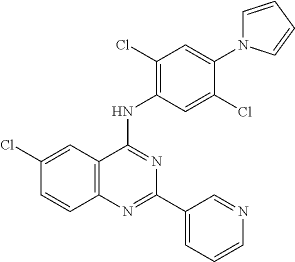 [SMILES: Clc1ccc2nc(-c3cccnc3)nc(Nc3cc(Cl)c(-n4cccc4)cc3Cl)c2c1] |
| 1000 | 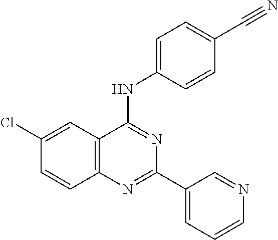 [SMILES: N#Cc1ccc(Nc2nc(-c3cccnc3)nc3ccc(Cl)cc23)cc1] |
| 1001 | 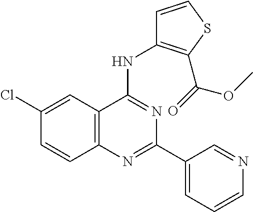 [SMILES: COC(=O)c1sccc1Nc1nc(-c2cccnc2)nc2ccc(Cl)cc12] |
| 1002 | 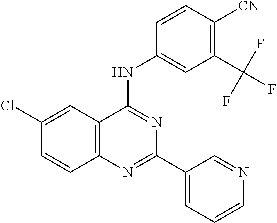 [SMILES: N#Cc1ccc(Nc2nc(-c3cccnc3)nc3ccc(Cl)cc23)cc1C(F)(F)F] |
| 1003 | 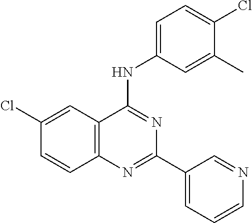 [SMILES: Cc1cc(Nc2nc(-c3cccnc3)nc3ccc(Cl)cc23)ccc1Cl] |
| 1004 | 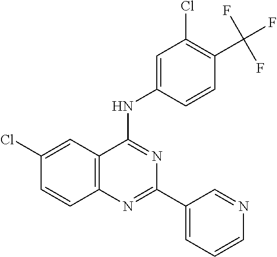 [SMILES: FC(F)(F)c1ccc(Nc2nc(-c3cccnc3)nc3ccc(Cl)cc23)cc1Cl] |
| 1005 | 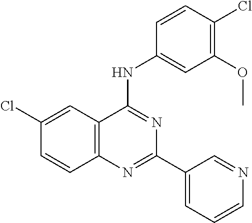 [SMILES: COc1cc(Nc2nc(-c3cccnc3)nc3ccc(Cl)cc23)ccc1Cl] |
| 1006 | 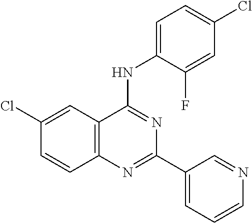 [SMILES: Fc1cc(Cl)ccc1Nc1nc(-c2cccnc2)nc2ccc(Cl)cc12] |
| 1007 | 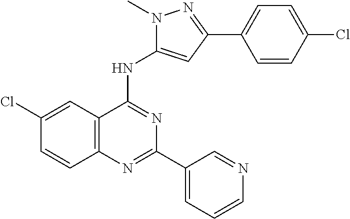 [SMILES: Cn1nc(-c2ccc(Cl)cc2)cc1Nc1nc(-c2cccnc2)nc2ccc(Cl)cc12] |
| 1008 | 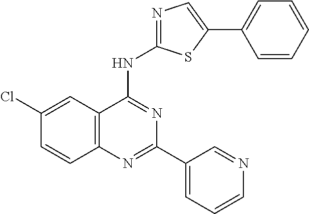 [SMILES: Clc1ccc2nc(-c3cccnc3)nc(Nc3ncc(-c4ccccc4)s3)c2c1] |
| 1009 | 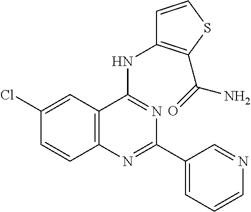 [SMILES: NC(=O)c1sccc1Nc1nc(-c2cccnc2)nc2ccc(Cl)cc12] |
| 1010 | 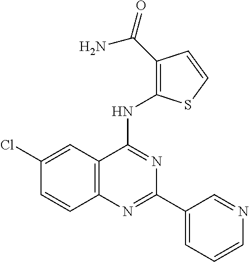 [SMILES: NC(=O)c1ccsc1Nc1nc(-c2cccnc2)nc2ccc(Cl)cc12] |
| 1013 | 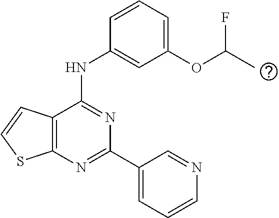 [SMILES: CC(F)Oc1cccc(Nc2nc(-c3cccnc3)nc3sccc23)c1] |
| 1015 | 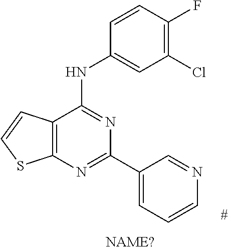 [SMILES: Fc1ccc(Nc2nc(-c3cccnc3)nc3sccc23)cc1Cl] |
| 1017 | 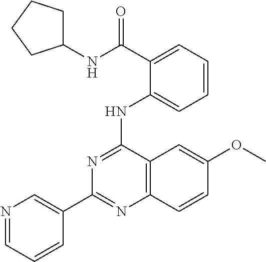 [SMILES: COc1ccc2nc(-c3cccnc3)nc(Nc3ccccc3C(=O)NC3CCCC3)c2c1] |
| 1019 | 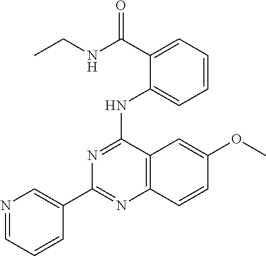 [SMILES: CCNC(=O)c1ccccc1Nc1nc(-c2cccnc2)nc2ccc(OC)cc12] |
| 1021 | 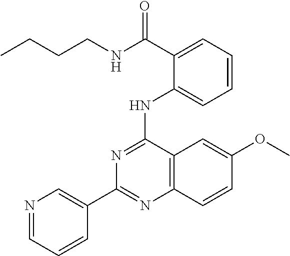 [SMILES: CCCCNC(=O)c1ccccc1Nc1nc(-c2cccnc2)nc2ccc(OC)cc12] |
| 1011 | 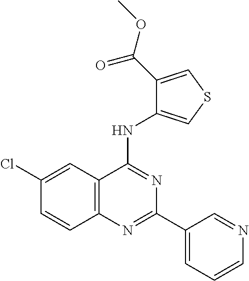 [SMILES: COC(=O)c1cscc1Nc1nc(-c2cccnc2)nc2ccc(Cl)cc12] |
| 1012 | 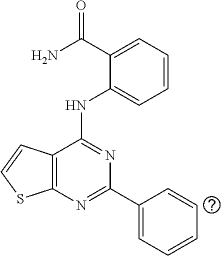 [SMILES: NC(=O)c1ccccc1Nc1nc(-c2ccccc2)nc2sccc12] |
| 1014 | 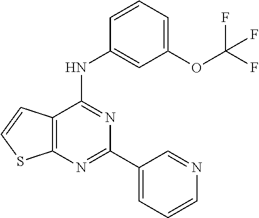 [SMILES: FC(F)(F)Oc1cccc(Nc2nc(-c3cccnc3)nc3sccc23)c1] |
| 1016 | 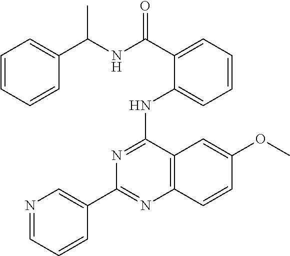 [SMILES: COc1ccc2nc(-c3cccnc3)nc(Nc3ccccc3C(=O)NC(C)c3ccccc3)c2c1] |
| 1018 | 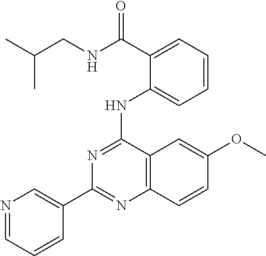 [SMILES: COc1ccc2nc(-c3cccnc3)nc(Nc3ccccc3C(=O)NCC(C)C)c2c1] |
| 1020 | 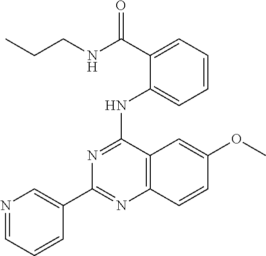 [SMILES: CCCNC(=O)c1ccccc1Nc1nc(-c2cccnc2)nc2ccc(OC)cc12] |
| 1022 | 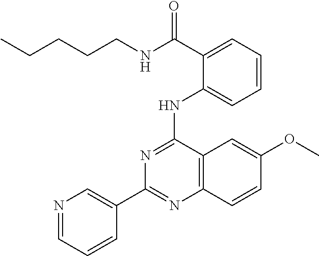 [SMILES: CCCCCNC(=O)c1ccccc1Nc1nc(-c2cccnc2)nc2ccc(OC)cc12] |
| 1023 | 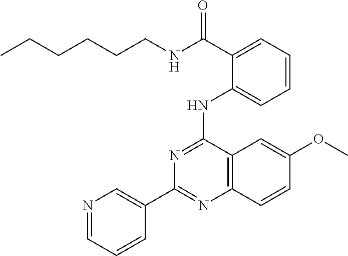 [SMILES: CCCCCCNC(=O)c1ccccc1Nc1nc(-c2cccnc2)nc2ccc(OC)cc12] |
| 1024 | 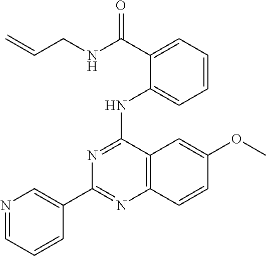 [SMILES: C=CCNC(=O)c1ccccc1Nc1nc(-c2cccnc2)nc2ccc(OC)cc12] |
| 1025 | 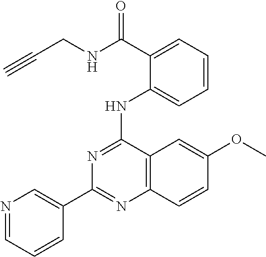 [SMILES: C#CCNC(=O)c1ccccc1Nc1nc(-c2cccnc2)nc2ccc(OC)cc12] |
| 1026 | 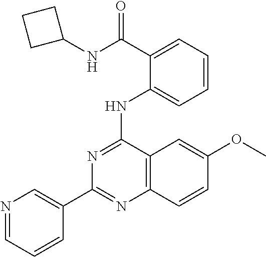 [SMILES: COc1ccc2nc(-c3cccnc3)nc(Nc3ccccc3C(=O)NC3CCC3)c2c1] |
| 1027 | 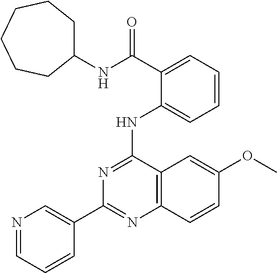 [SMILES: COc1ccc2nc(-c3cccnc3)nc(Nc3ccccc3C(=O)NC3CCCCCC3)c2c1] |
| 1028 | 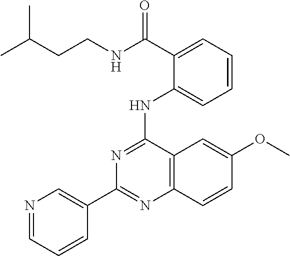 [SMILES: COc1ccc2nc(-c3cccnc3)nc(Nc3ccccc3C(=O)NCCC(C)C)c2c1] |
| 1029 | 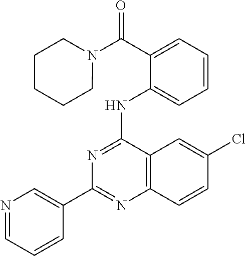 [SMILES: O=C(c1ccccc1Nc1nc(-c2cccnc2)nc2ccc(Cl)cc12)N1CCCCC1] |
| 1030 | 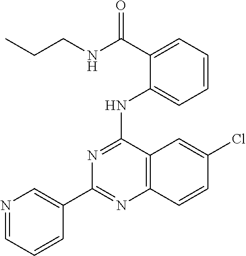 [SMILES: CCCNC(=O)c1ccccc1Nc1nc(-c2cccnc2)nc2ccc(Cl)cc12] |
| 1031 | 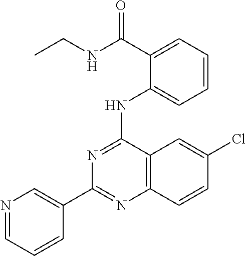 [SMILES: CCNC(=O)c1ccccc1Nc1nc(-c2cccnc2)nc2ccc(Cl)cc12] |
| 1032 | 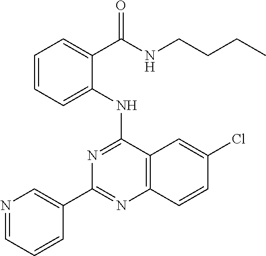 [SMILES: CCCCNC(=O)c1ccccc1Nc1nc(-c2cccnc2)nc2ccc(Cl)cc12] |
| 1033 | 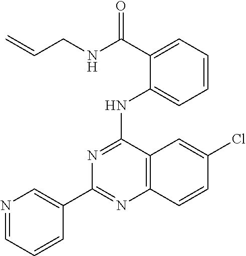 [SMILES: C=CCNC(=O)c1ccccc1Nc1nc(-c2cccnc2)nc2ccc(Cl)cc12] |
| 1034 | 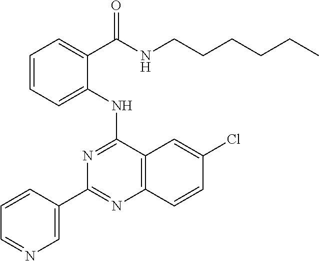 [SMILES: CCCCCCNC(=O)c1ccccc1Nc1nc(-c2cccnc2)nc2ccc(Cl)cc12] |
| 1035 | 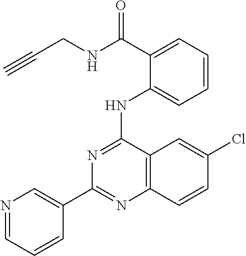 [SMILES: C#CCNC(=O)c1ccccc1Nc1nc(-c2cccnc2)nc2ccc(Cl)cc12] |
| 1036 | 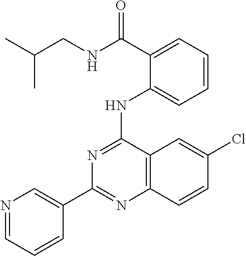 [SMILES: CC(C)CNC(=O)c1ccccc1Nc1nc(-c2cccnc2)nc2ccc(Cl)cc12] |
| 1037 | 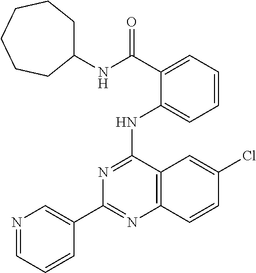 [SMILES: O=C(NC1CCCCCC1)c1ccccc1Nc1nc(-c2cccnc2)nc2ccc(Cl)cc12] |
| 1038 | 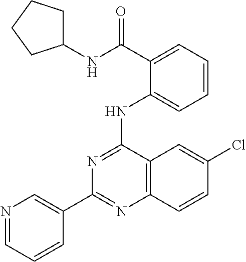 [SMILES: O=C(NC1CCCC1)c1ccccc1Nc1nc(-c2cccnc2)nc2ccc(Cl)cc12] |
| 1039 | 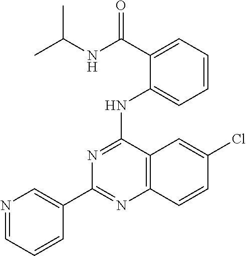 [SMILES: CC(C)NC(=O)c1ccccc1Nc1nc(-c2cccnc2)nc2ccc(Cl)cc12] |
| 1040 | 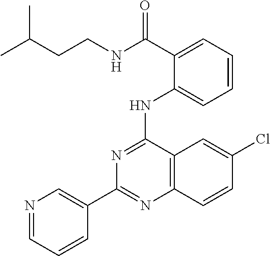 [SMILES: CC(C)CCNC(=O)c1ccccc1Nc1nc(-c2cccnc2)nc2ccc(Cl)cc12] |
| 1041 | 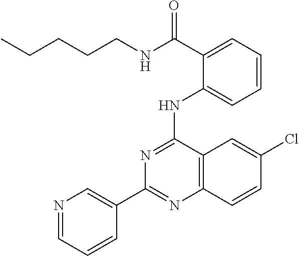 [SMILES: CCCCCNC(=O)c1ccccc1Nc1nc(-c2cccnc2)nc2ccc(Cl)cc12] |
| 1042 | 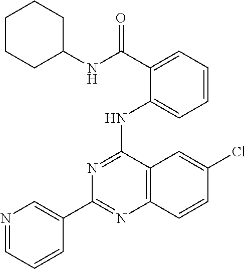 [SMILES: O=C(NC1CCCCC1)c1ccccc1Nc1nc(-c2cccnc2)nc2ccc(Cl)cc12] |
| 1043 | 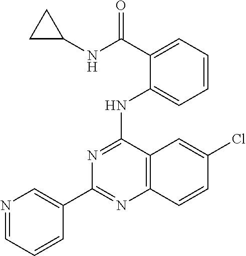 [SMILES: O=C(NC1CC1)c1ccccc1Nc1nc(-c2cccnc2)nc2ccc(Cl)cc12] |
| 1044 | 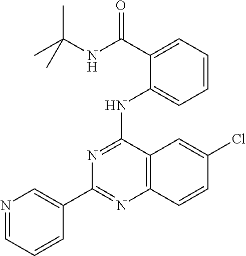 [SMILES: CC(C)(C)NC(=O)c1ccccc1Nc1nc(-c2cccnc2)nc2ccc(Cl)cc12] |
| 1045 | 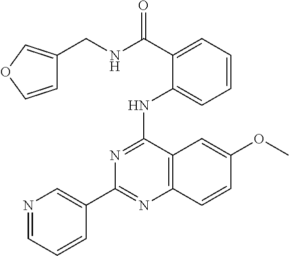 [SMILES: COc1ccc2nc(-c3cccnc3)nc(Nc3ccccc3C(=O)NCc3ccoc3)c2c1] |
| 1046 | 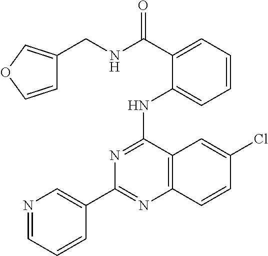 [SMILES: O=C(NCc1ccoc1)c1ccccc1Nc1nc(-c2cccnc2)nc2ccc(Cl)cc12] |
| 1047 | 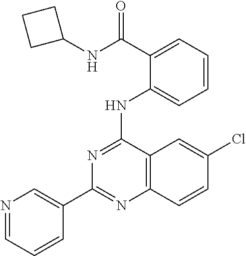 [SMILES: O=C(NC1CCC1)c1ccccc1Nc1nc(-c2cccnc2)nc2ccc(Cl)cc12] |
| 1048 | 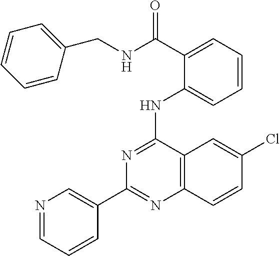 [SMILES: O=C(NCc1ccccc1)c1ccccc1Nc1nc(-c2cccnc2)nc2ccc(Cl)cc12] |
| 1049 | 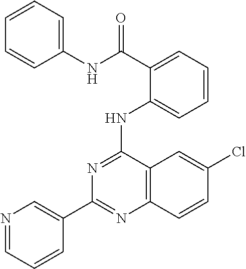 [SMILES: O=C(Nc1ccccc1)c1ccccc1Nc1nc(-c2cccnc2)nc2ccc(Cl)cc12] |
| 1050 | 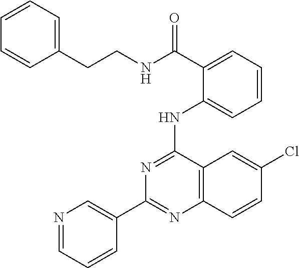 [SMILES: O=C(NCCc1ccccc1)c1ccccc1Nc1nc(-c2cccnc2)nc2ccc(Cl)cc12] |
| 1051 | 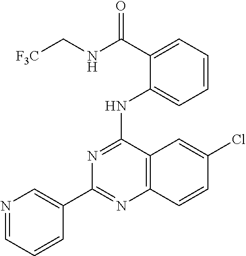 [SMILES: O=C(NCC(F)(F)F)c1ccccc1Nc1nc(-c2cccnc2)nc2ccc(Cl)cc12] |
| 1052 | 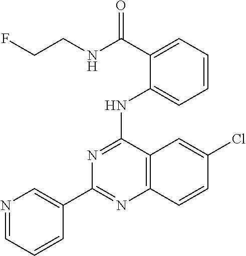 [SMILES: O=C(NCCF)c1ccccc1Nc1nc(-c2cccnc2)nc2ccc(Cl)cc12] |
| 1053 | 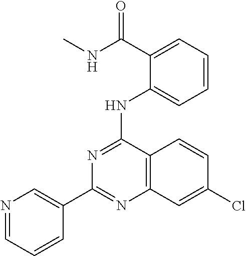 [SMILES: CNC(=O)c1ccccc1Nc1nc(-c2cccnc2)nc2cc(Cl)ccc12] |
| 1054 | 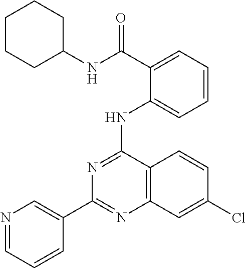 [SMILES: O=C(NC1CCCCC1)c1ccccc1Nc1nc(-c2cccnc2)nc2cc(Cl)ccc12] |
| 1055 | 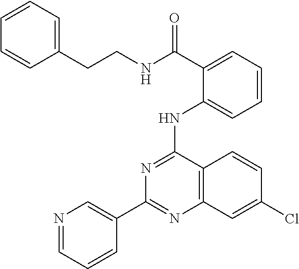 [SMILES: O=C(NCCc1ccccc1)c1ccccc1Nc1nc(-c2cccnc2)nc2cc(Cl)ccc12] |
| 1056 | 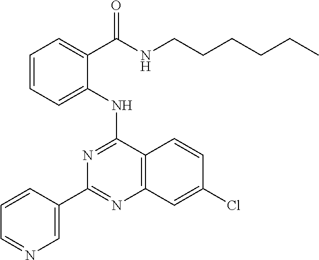 [SMILES: CCCCCCNC(=O)c1ccccc1Nc1nc(-c2cccnc2)nc2cc(Cl)ccc12] |
| 1057 | 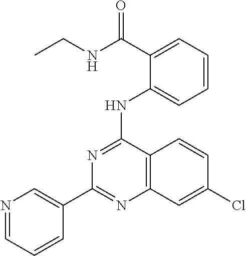 [SMILES: CCNC(=O)c1ccccc1Nc1nc(-c2cccnc2)nc2cc(Cl)ccc12] |
| 1058 | 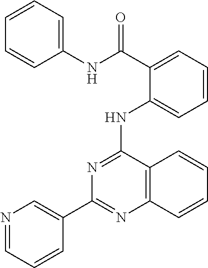 [SMILES: O=C(Nc1ccccc1)c1ccccc1Nc1nc(-c2cccnc2)nc2ccccc12] |
| 1059 | 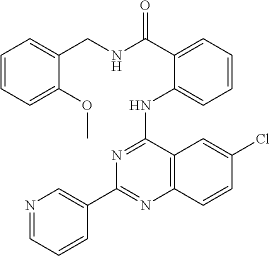 [SMILES: COc1ccccc1CNC(=O)c1ccccc1Nc1nc(-c2cccnc2)nc2ccc(Cl)cc12] |
| 1060 | 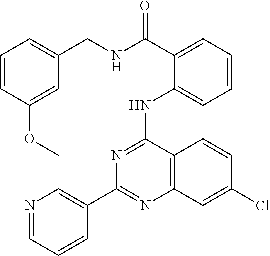 [SMILES: COc1cccc(CNC(=O)c2ccccc2Nc2nc(-c3cccnc3)nc3cc(Cl)ccc23)c1] |
| 1061 | 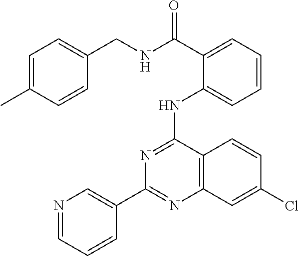 [SMILES: Cc1ccc(CNC(=O)c2ccccc2Nc2nc(-c3cccnc3)nc3cc(Cl)ccc23)cc1] |
| 1062 | 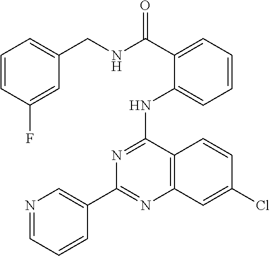 [SMILES: O=C(NCc1cccc(F)c1)c1ccccc1Nc1nc(-c2cccnc2)nc2cc(Cl)ccc12] |
| 1063 | 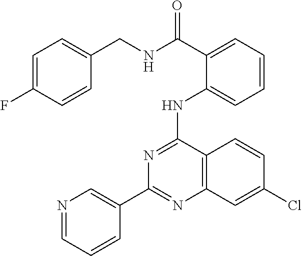 [SMILES: O=C(NCc1ccc(F)cc1)c1ccccc1Nc1nc(-c2cccnc2)nc2cc(Cl)ccc12] |
| 1064 | 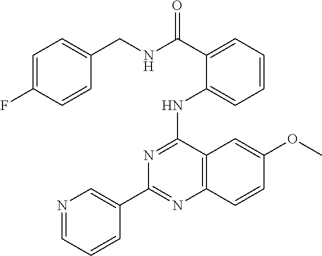 [SMILES: COc1ccc2nc(-c3cccnc3)nc(Nc3ccccc3C(=O)NCc3ccc(F)cc3)c2c1] |
| 1065 | 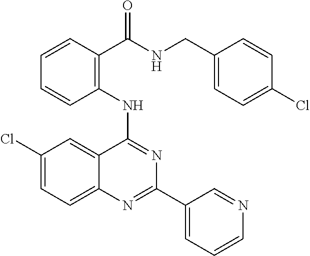 [SMILES: O=C(NCc1ccc(Cl)cc1)c1ccccc1Nc1nc(-c2cccnc2)nc2ccc(Cl)cc12] |
| 1066 | 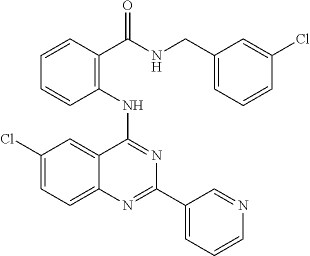 [SMILES: O=C(NCc1cccc(Cl)c1)c1ccccc1Nc1nc(-c2cccnc2)nc2ccc(Cl)cc12] |
| 1067 | 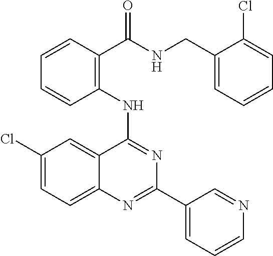 [SMILES: O=C(NCc1ccccc1Cl)c1ccccc1Nc1nc(-c2cccnc2)nc2ccc(Cl)cc12] |
| 1068 | 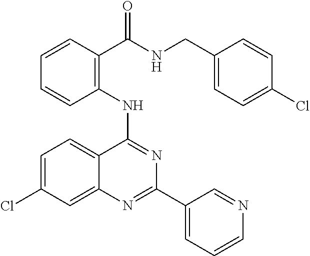 [SMILES: O=C(NCc1ccc(Cl)cc1)c1ccccc1Nc1nc(-c2cccnc2)nc2cc(Cl)ccc12] |
| 1069 | 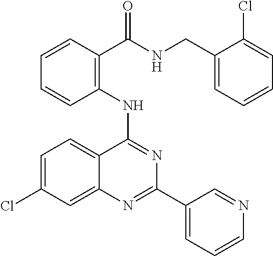 [SMILES: O=C(NCc1ccccc1Cl)c1ccccc1Nc1nc(-c2cccnc2)nc2cc(Cl)ccc12] |
| 1070 | 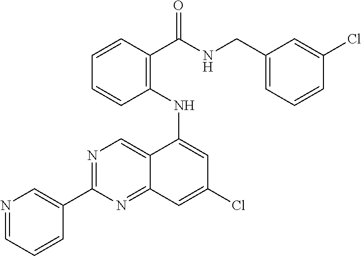 [SMILES: O=C(NCc1cccc(Cl)c1)c1ccccc1Nc1cc(Cl)cc2nc(-c3cccnc3)ncc12] |
| 1071 | 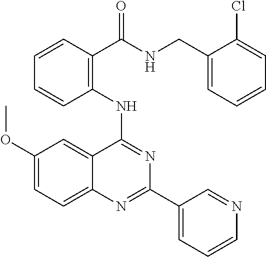 [SMILES: COc1ccc2nc(-c3cccnc3)nc(Nc3ccccc3C(=O)NCc3ccccc3Cl)c2c1] |
| 1072 | 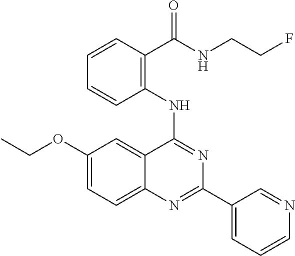 [SMILES: CCOc1ccc2nc(-c3cccnc3)nc(Nc3ccccc3C(=O)NCCF)c2c1] |
| 1073 | 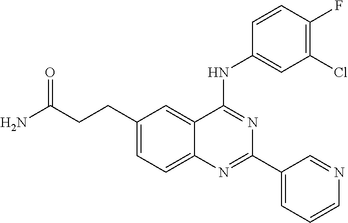 [SMILES: NC(=O)CCc1ccc2nc(-c3cccnc3)nc(Nc3ccc(F)c(Cl)c3)c2c1] |
| 1074 | 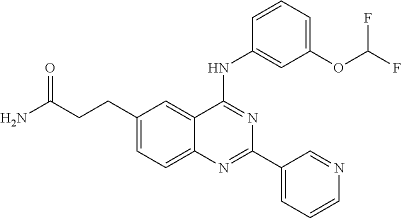 [SMILES: NC(=O)CCc1ccc2nc(-c3cccnc3)nc(Nc3cccc(OC(F)F)c3)c2c1] |
| 1075 | 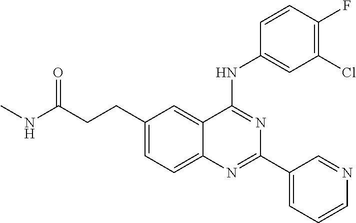 [SMILES: CNC(=O)CCc1ccc2nc(-c3cccnc3)nc(Nc3ccc(F)c(Cl)c3)c2c1] |
| 1076 | 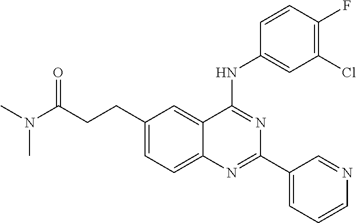 [SMILES: CN(C)C(=O)CCc1ccc2nc(-c3cccnc3)nc(Nc3ccc(F)c(Cl)c3)c2c1] |
| 1077 | 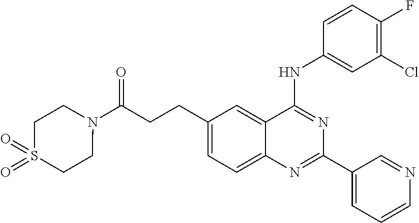 [SMILES: O=C(CCc1ccc2nc(-c3cccnc3)nc(Nc3ccc(F)c(Cl)c3)c2c1)N1CCS(=O)(=O)CC1] |
| 1078 | 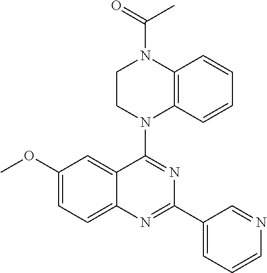 [SMILES: COc1ccc2nc(-c3cccnc3)nc(N3CCN(C(C)=O)c4ccccc43)c2c1] |
| 1079 | 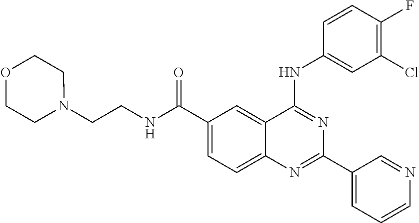 [SMILES: O=C(NCCN1CCOCC1)c1ccc2nc(-c3cccnc3)nc(Nc3ccc(F)c(Cl)c3)c2c1] |
| 1080 | 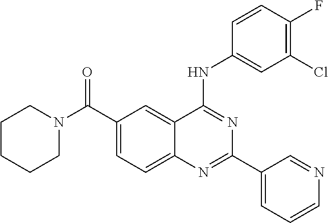 [SMILES: O=C(c1ccc2nc(-c3cccnc3)nc(Nc3ccc(F)c(Cl)c3)c2c1)N1CCCCC1] |
| 1081 | 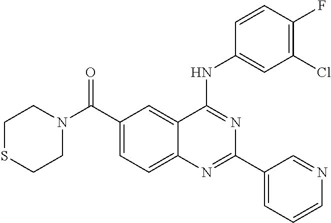 [SMILES: O=C(c1ccc2nc(-c3cccnc3)nc(Nc3ccc(F)c(Cl)c3)c2c1)N1CCSCC1] |
| 1082 | 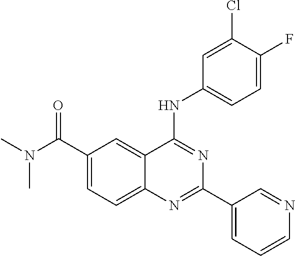 [SMILES: CN(C)C(=O)c1ccc2nc(-c3cccnc3)nc(Nc3ccc(F)c(Cl)c3)c2c1] |
| 1083 | 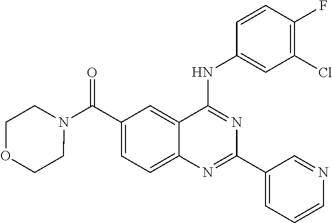 [SMILES: O=C(c1ccc2nc(-c3cccnc3)nc(Nc3ccc(F)c(Cl)c3)c2c1)N1CCOCC1] |
| 1084 | 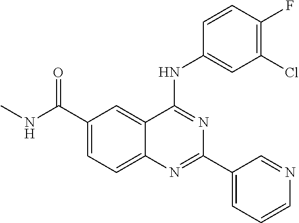 [SMILES: CNC(=O)c1ccc2nc(-c3cccnc3)nc(Nc3ccc(F)c(Cl)c3)c2c1] |
| 1085 | 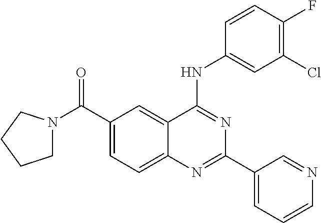 [SMILES: O=C(c1ccc2nc(-c3cccnc3)nc(Nc3ccc(F)c(Cl)c3)c2c1)N1CCCC1] |
| 1086 | 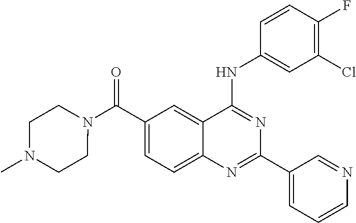 [SMILES: CN1CCN(C(=O)c2ccc3nc(-c4cccnc4)nc(Nc4ccc(F)c(Cl)c4)c3c2)CC1] |
| 1087 | 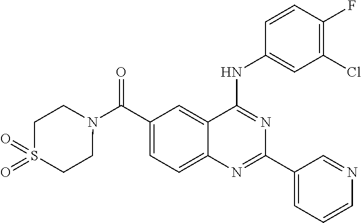 [SMILES: O=C(c1ccc2nc(-c3cccnc3)nc(Nc3ccc(F)c(Cl)c3)c2c1)N1CCS(=O)(=O)CC1] |
| 1088 | 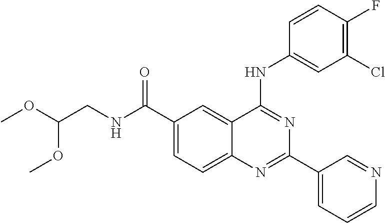 [SMILES: COC(CNC(=O)c1ccc2nc(-c3cccnc3)nc(Nc3ccc(F)c(Cl)c3)c2c1)OC] |
| 1089 | 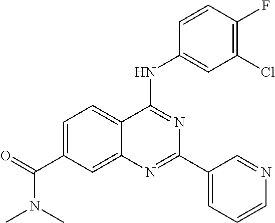 [SMILES: CN(C)C(=O)c1ccc2c(Nc3ccc(F)c(Cl)c3)nc(-c3cccnc3)nc2c1] |
| 1090 | 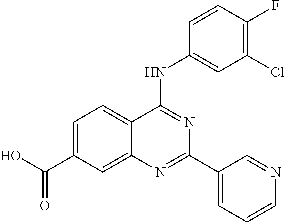 [SMILES: O=C(O)c1ccc2c(Nc3ccc(F)c(Cl)c3)nc(-c3cccnc3)nc2c1] |
| 1091 | 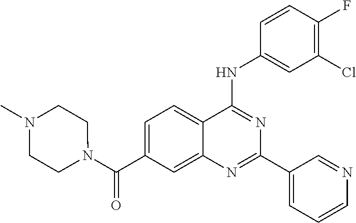 [SMILES: CN1CCN(C(=O)c2ccc3c(Nc4ccc(F)c(Cl)c4)nc(-c4cccnc4)nc3c2)CC1] |
| 1092 | 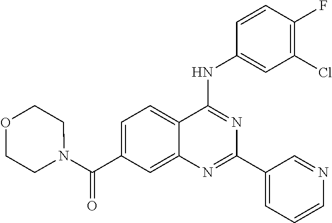 [SMILES: O=C(c1ccc2c(Nc3ccc(F)c(Cl)c3)nc(-c3cccnc3)nc2c1)N1CCOCC1] |
| 1093 | 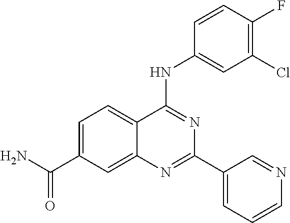 [SMILES: NC(=O)c1ccc2c(Nc3ccc(F)c(Cl)c3)nc(-c3cccnc3)nc2c1] |
| 1094 | 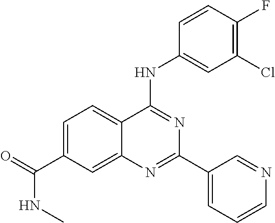 [SMILES: CNC(=O)c1ccc2c(Nc3ccc(F)c(Cl)c3)nc(-c3cccnc3)nc2c1] |
| 1095 | 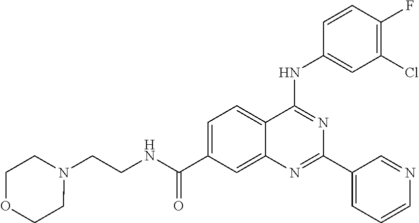 [SMILES: O=C(NCCN1CCOCC1)c1ccc2c(Nc3ccc(F)c(Cl)c3)nc(-c3cccnc3)nc2c1] |
| 1096 | 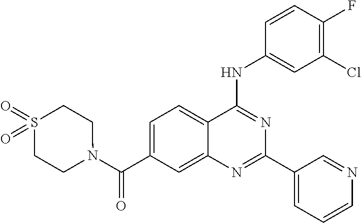 [SMILES: O=C(c1ccc2c(Nc3ccc(F)c(Cl)c3)nc(-c3cccnc3)nc2c1)N1CCS(=O)(=O)CC1] |
| 1097 | 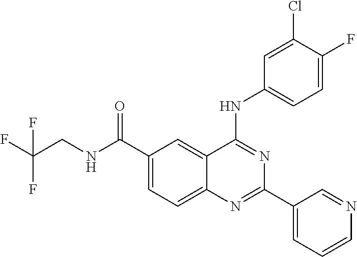 [SMILES: O=C(NCC(F)(F)F)c1ccc2nc(-c3cccnc3)nc(Nc3ccc(F)c(Cl)c3)c2c1] |
| 1098 | 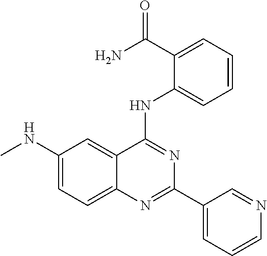 [SMILES: CNc1ccc2nc(-c3cccnc3)nc(Nc3ccccc3C(N)=O)c2c1] |
| 1099 | 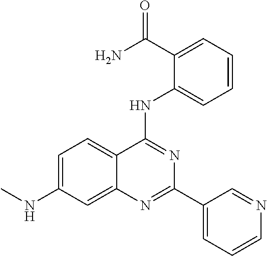 [SMILES: CNc1ccc2c(Nc3ccccc3C(N)=O)nc(-c3cccnc3)nc2c1] |
| 1100 | 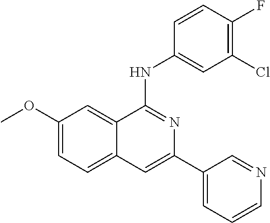 [SMILES: COc1ccc2cc(-c3cccnc3)nc(Nc3ccc(F)c(Cl)c3)c2c1] |
| 1101 | 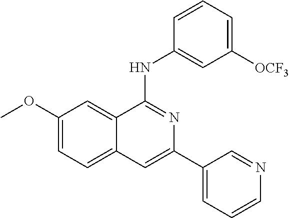 [SMILES: COc1ccc2cc(-c3cccnc3)nc(Nc3cccc(OC(F)(F)F)c3)c2c1] |
| 1102 | 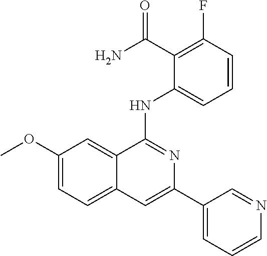 [SMILES: COc1ccc2cc(-c3cccnc3)nc(Nc3cccc(F)c3C(N)=O)c2c1] |
| 1103 | 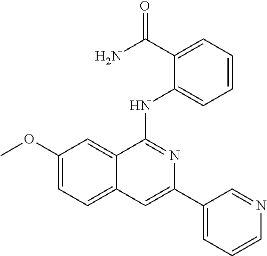 [SMILES: COc1ccc2cc(-c3cccnc3)nc(Nc3ccccc3C(N)=O)c2c1] |
| 1104 | 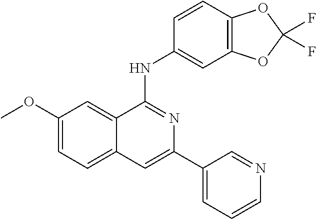 [SMILES: COc1ccc2cc(-c3cccnc3)nc(Nc3ccc4c(c3)OC(F)(F)O4)c2c1] |
| 1105 | 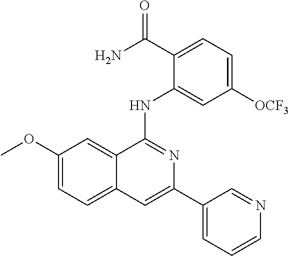 [SMILES: COc1ccc2cc(-c3cccnc3)nc(Nc3cc(OC(F)(F)F)ccc3C(N)=O)c2c1] |
| 1106 | 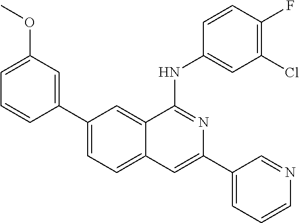 [SMILES: COc1cccc(-c2ccc3cc(-c4cccnc4)nc(Nc4ccc(F)c(Cl)c4)c3c2)c1] |
| 1107 | 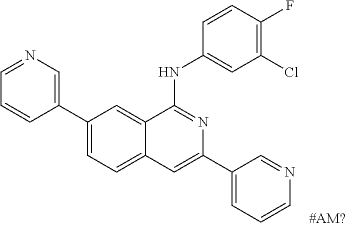 [SMILES: Fc1ccc(Nc2nc(-c3cccnc3)cc3ccc(-c4cccnc4)cc23)cc1Cl] |
| 1108 | 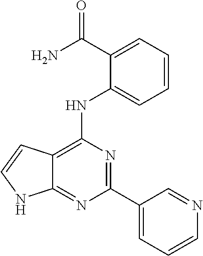 [SMILES: NC(=O)c1ccccc1Nc1nc(-c2cccnc2)nc2[nH]ccc12] |
| 1109 | 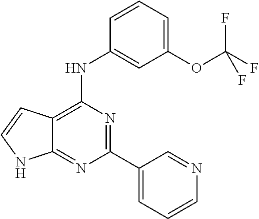 [SMILES: FC(F)(F)Oc1cccc(Nc2nc(-c3cccnc3)nc3[nH]ccc23)c1] |
| 1110 | 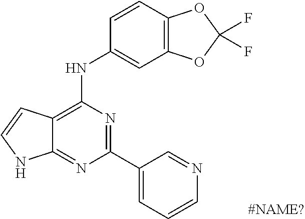 [SMILES: FC1(F)Oc2ccc(Nc3nc(-c4cccnc4)nc4[nH]ccc34)cc2O1] |
| 1111 | 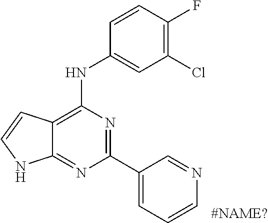 [SMILES: Fc1ccc(Nc2nc(-c3cccnc3)nc3[nH]ccc23)cc1Cl] |
| 1112 | 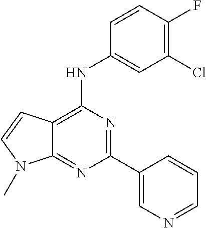 [SMILES: Cn1ccc2c(Nc3ccc(F)c(Cl)c3)nc(-c3cccnc3)nc21] |
| 1113 | 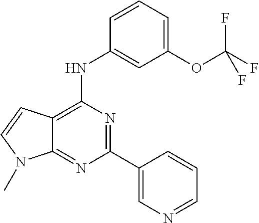 [SMILES: Cn1ccc2c(Nc3cccc(OC(F)(F)F)c3)nc(-c3cccnc3)nc21] |
| 1114 | 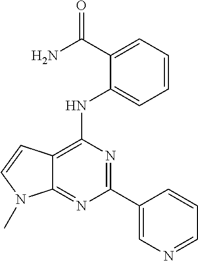 [SMILES: Cn1ccc2c(Nc3ccccc3C(N)=O)nc(-c3cccnc3)nc21] |
| 1115 | 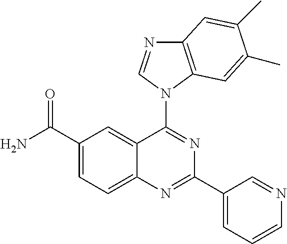 [SMILES: Cc1cc2ncn(-c3nc(-c4cccnc4)nc4ccc(C(N)=O)cc34)c2cc1C] |
| 1116 | 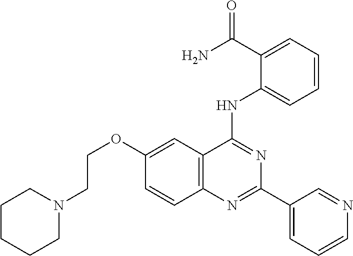 [SMILES: NC(=O)c1ccccc1Nc1nc(-c2cccnc2)nc2ccc(OCCN3CCCCC3)cc12] |
| 1117 | 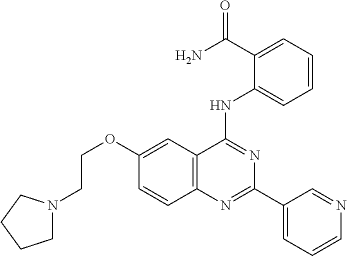 [SMILES: NC(=O)c1ccccc1Nc1nc(-c2cccnc2)nc2ccc(OCCN3CCCC3)cc12] |
| 1118 | 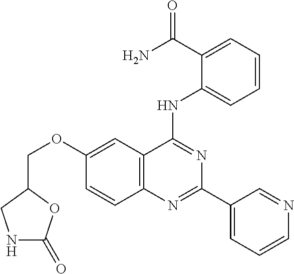 [SMILES: NC(=O)c1ccccc1Nc1nc(-c2cccnc2)nc2ccc(OCC3CNC(=O)O3)cc12] |
| 1119 | 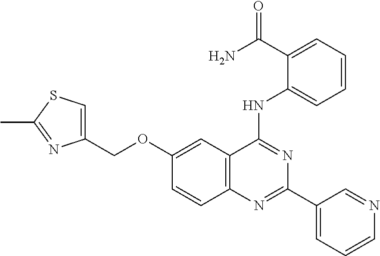 [SMILES: Cc1nc(COc2ccc3nc(-c4cccnc4)nc(Nc4ccccc4C(N)=O)c3c2)cs1] |
| 1120 | 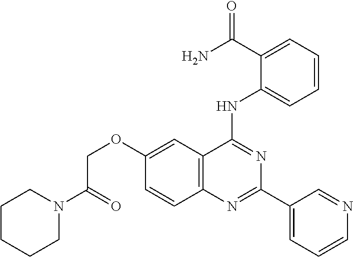 [SMILES: NC(=O)c1ccccc1Nc1nc(-c2cccnc2)nc2ccc(OCC(=O)N3CCCCC3)cc12] |
| 1121 | 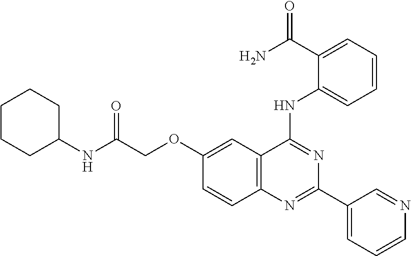 [SMILES: NC(=O)c1ccccc1Nc1nc(-c2cccnc2)nc2ccc(OCC(=O)NC3CCCCC3)cc12] |
| 1122 | 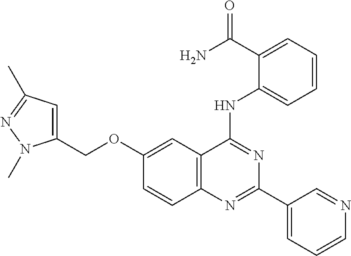 [SMILES: Cc1cc(COc2ccc3nc(-c4cccnc4)nc(Nc4ccccc4C(N)=O)c3c2)n(C)n1] |
| 1123 | 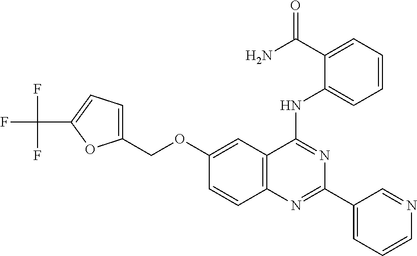 [SMILES: NC(=O)c1ccccc1Nc1nc(-c2cccnc2)nc2ccc(OCc3ccc(C(F)(F)F)o3)cc12] |
| 1124 | 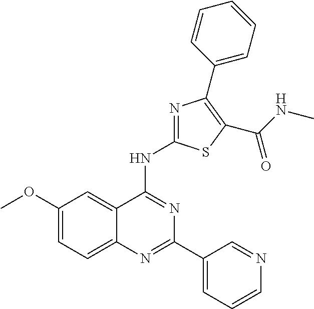 |
| 1125 | 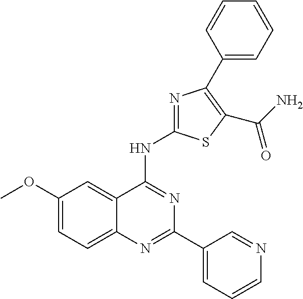 [SMILES: COc1ccc2nc(-c3cccnc3)nc(Nc3nc(-c4ccccc4)c(C(N)=O)s3)c2c1] |
| 1126 | 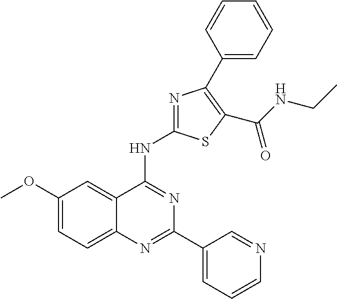 [SMILES: CCNC(=O)c1sc(Nc2nc(-c3cccnc3)nc3ccc(OC)cc23)nc1-c1ccccc1] |
| 1127 | 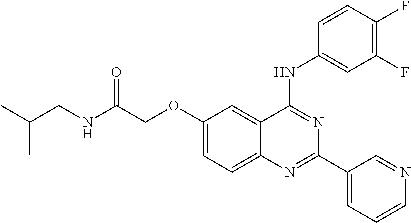 [SMILES: CC(C)CNC(=O)COc1ccc2nc(-c3cccnc3)nc(Nc3ccc(F)c(F)c3)c2c1] |
| 1128 | 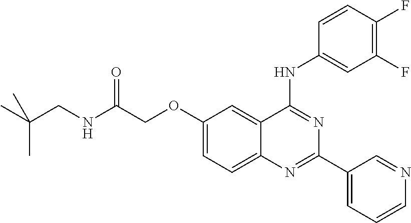 [SMILES: CC(C)(C)CNC(=O)COc1ccc2nc(-c3cccnc3)nc(Nc3ccc(F)c(F)c3)c2c1] |
| 1129 | 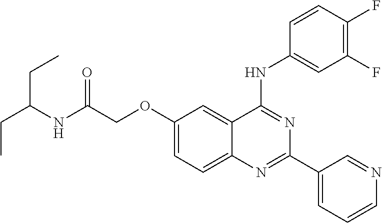 [SMILES: CCC(CC)NC(=O)COc1ccc2nc(-c3cccnc3)nc(Nc3ccc(F)c(F)c3)c2c1] |
| 1130 | 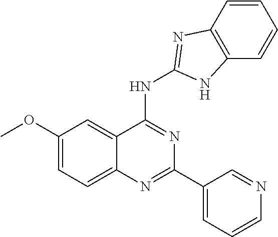 [SMILES: COc1ccc2nc(-c3cccnc3)nc(Nc3nc4ccccc4[nH]3)c2c1] |
| 1131 | 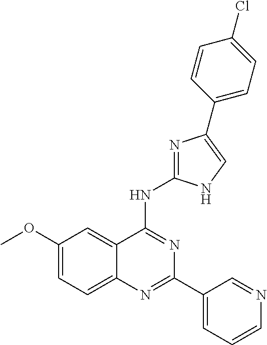 [SMILES: COc1ccc2nc(-c3cccnc3)nc(Nc3nc(-c4ccc(Cl)cc4)c[nH]3)c2c1] |
| 1132 | 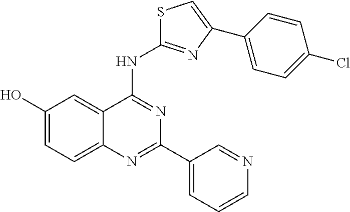 [SMILES: Oc1ccc2nc(-c3cccnc3)nc(Nc3nc(-c4ccc(Cl)cc4)cs3)c2c1] |
| 1133 | 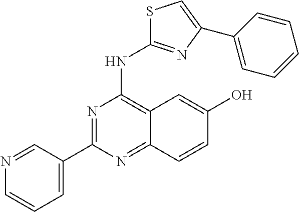 [SMILES: Oc1ccc2nc(-c3cccnc3)nc(Nc3nc(-c4ccccc4)cs3)c2c1] |
| 1134 | 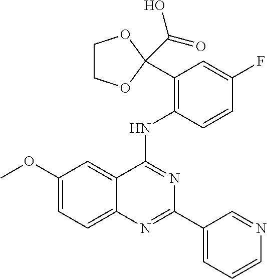 [SMILES: COc1ccc2nc(-c3cccnc3)nc(Nc3ccc(F)cc3C3(C(=O)O)OCCO3)c2c1] |
| 1135 | 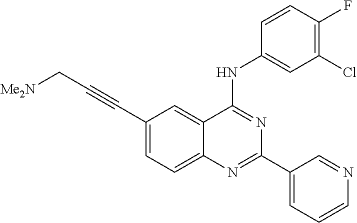 [SMILES: CN(C)CC#Cc1ccc2nc(-c3cccnc3)nc(Nc3ccc(F)c(Cl)c3)c2c1] |
| 1136 | 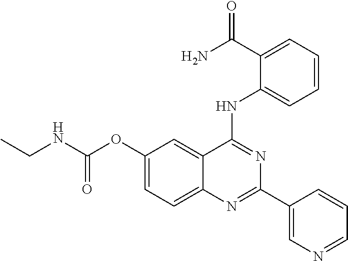 [SMILES: CCNC(=O)Oc1ccc2nc(-c3cccnc3)nc(Nc3ccccc3C(N)=O)c2c1] |
| 1137 | 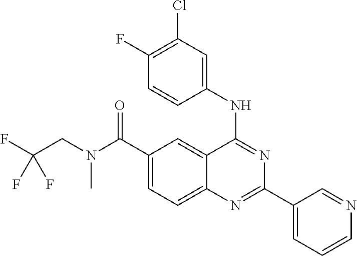 [SMILES: CN(CC(F)(F)F)C(=O)c1ccc2nc(-c3cccnc3)nc(Nc3ccc(F)c(Cl)c3)c2c1] |
| 1756 | 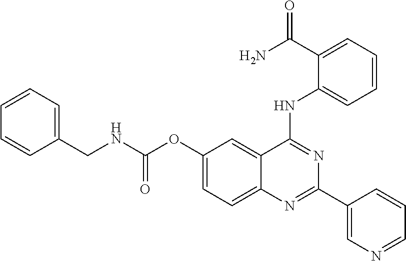 [SMILES: NC(=O)c1ccccc1Nc1nc(-c2cccnc2)nc2ccc(OC(=O)NCc3ccccc3)cc12] |
| 1140 | 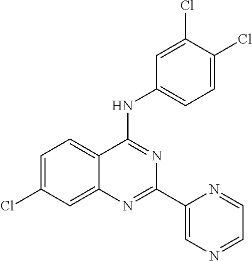 [SMILES: Clc1ccc2c(Nc3ccc(Cl)c(Cl)c3)nc(-c3cnccn3)nc2c1] |
| 1141 | 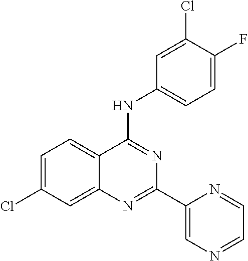 [SMILES: Fc1ccc(Nc2nc(-c3cnccn3)nc3cc(Cl)ccc23)cc1Cl] |
| 1138 | 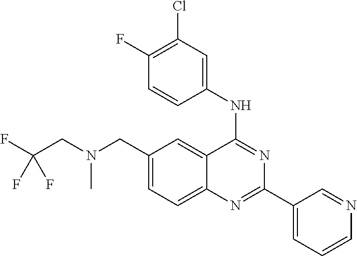 [SMILES: CN(Cc1ccc2nc(-c3cccnc3)nc(Nc3ccc(F)c(Cl)c3)c2c1)CC(F)(F)F] |
| 1139 | 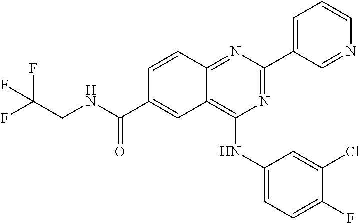 [SMILES: O=C(NCC(F)(F)F)c1ccc2nc(-c3cccnc3)nc(Nc3ccc(F)c(Cl)c3)c2c1] |
| 1142 | 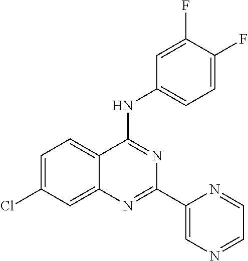 [SMILES: Fc1ccc(Nc2nc(-c3cnccn3)nc3cc(Cl)ccc23)cc1F] |
| 1143 | 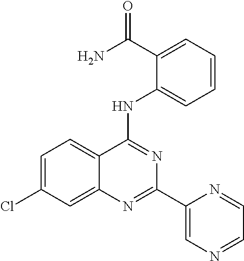 [SMILES: NC(=O)c1ccccc1Nc1nc(-c2cnccn2)nc2cc(Cl)ccc12] |
| 1144 | 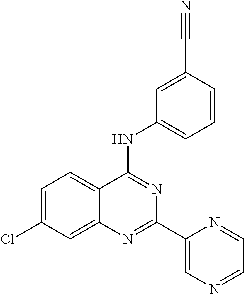 [SMILES: N#Cc1cccc(Nc2nc(-c3cnccn3)nc3cc(Cl)ccc23)c1] |
| 1145 | 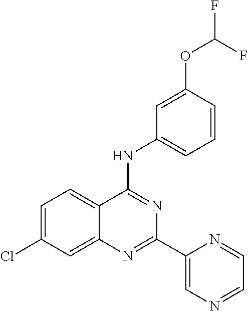 [SMILES: FC(F)Oc1cccc(Nc2nc(-c3cnccn3)nc3cc(Cl)ccc23)c1] |
| 1146 | 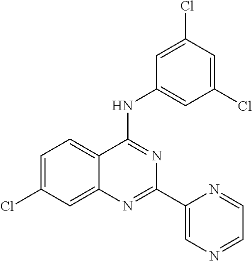 [SMILES: Clc1cc(Cl)cc(Nc2nc(-c3cnccn3)nc3cc(Cl)ccc23)c1] |
| 1147 | 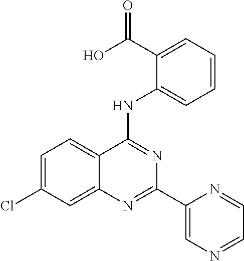 [SMILES: O=C(O)c1ccccc1Nc1nc(-c2cnccn2)nc2cc(Cl)ccc12] |
| 1148 | 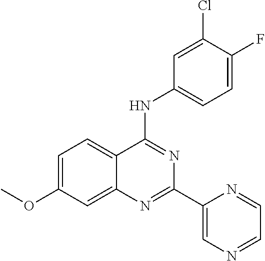 [SMILES: COc1ccc2c(Nc3ccc(F)c(Cl)c3)nc(-c3cnccn3)nc2c1] |
| 1149 | 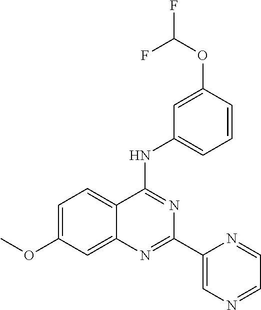 [SMILES: COc1ccc2c(Nc3cccc(OC(F)F)c3)nc(-c3cnccn3)nc2c1] |
| 1150 | 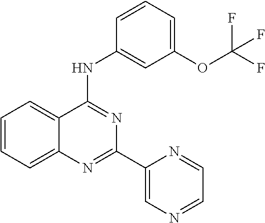 [SMILES: FC(F)(F)Oc1cccc(Nc2nc(-c3cnccn3)nc3ccccc23)c1] |
| 1151 | 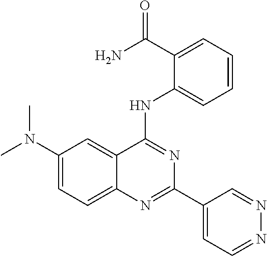 [SMILES: CN(C)c1ccc2nc(-c3ccnnc3)nc(Nc3ccccc3C(N)=O)c2c1] |
| 1152 | 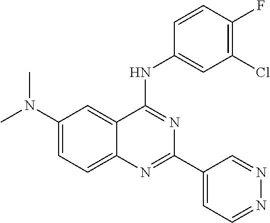 [SMILES: CN(C)c1ccc2nc(-c3ccnnc3)nc(Nc3ccc(F)c(Cl)c3)c2c1] |
| 1153 | 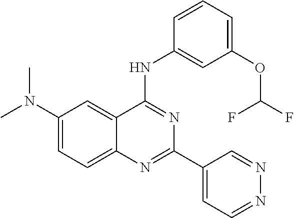 [SMILES: CN(C)c1ccc2nc(-c3ccnnc3)nc(Nc3cccc(OC(F)F)c3)c2c1] |
| 1154 | 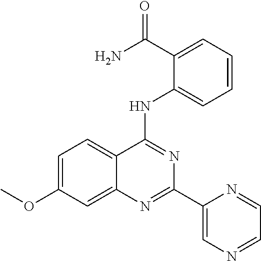 [SMILES: COc1ccc2c(Nc3ccccc3C(N)=O)nc(-c3cnccn3)nc2c1] |
| 1155 | 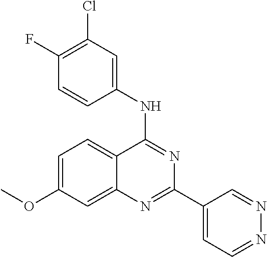 [SMILES: COc1ccc2c(Nc3ccc(F)c(Cl)c3)nc(-c3ccnnc3)nc2c1] |
| 1156 | 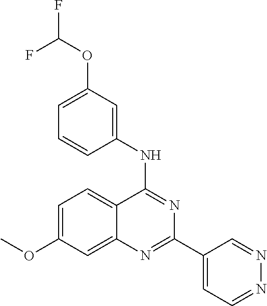 [SMILES: COc1ccc2c(Nc3cccc(OC(F)F)c3)nc(-c3ccnnc3)nc2c1] |
| 1157 | 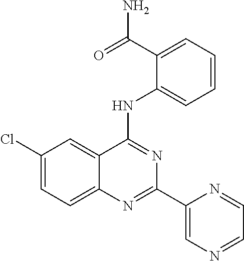 [SMILES: NC(=O)c1ccccc1Nc1nc(-c2cnccn2)nc2ccc(Cl)cc12] |
| 1158 | 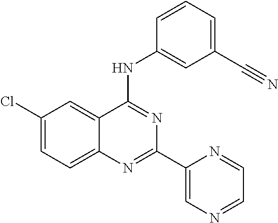 [SMILES: N#Cc1cccc(Nc2nc(-c3cnccn3)nc3ccc(Cl)cc23)c1] |
| 1159 | 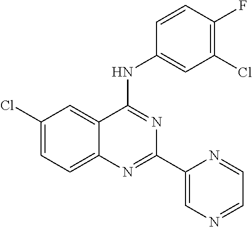 [SMILES: Fc1ccc(Nc2nc(-c3cnccn3)nc3ccc(Cl)cc23)cc1Cl] |
| 1160 | 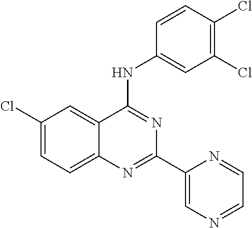 [SMILES: Clc1ccc2nc(-c3cnccn3)nc(Nc3ccc(Cl)c(Cl)c3)c2c1] |
| 1161 | 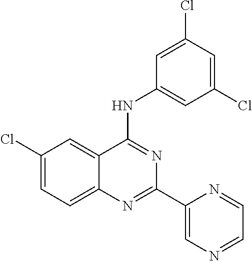 [SMILES: Clc1cc(Cl)cc(Nc2nc(-c3cnccn3)nc3ccc(Cl)cc23)c1] |
| 1162 | 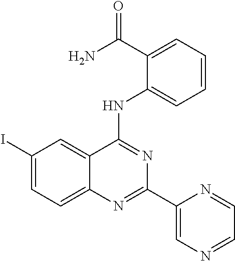 [SMILES: NC(=O)c1ccccc1Nc1nc(-c2cnccn2)nc2ccc(I)cc12] |
| 1163 | 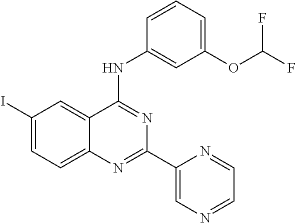 [SMILES: FC(F)Oc1cccc(Nc2nc(-c3cnccn3)nc3ccc(I)cc23)c1] |
| 1164 | 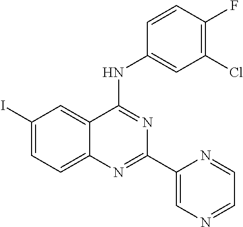 [SMILES: Fc1ccc(Nc2nc(-c3cnccn3)nc3ccc(I)cc23)cc1Cl] |
| 1165 | 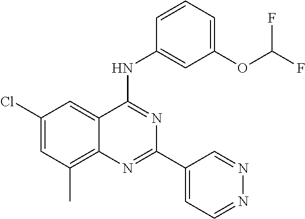 [SMILES: Cc1cc(Cl)cc2c(Nc3cccc(OC(F)F)c3)nc(-c3ccnnc3)nc12] |
| 1166 | 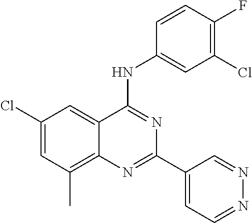 [SMILES: Cc1cc(Cl)cc2c(Nc3ccc(F)c(Cl)c3)nc(-c3ccnnc3)nc12] |
| 1167 | 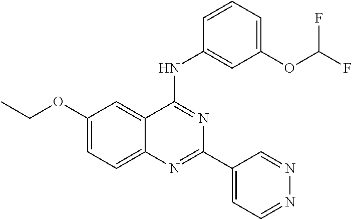 [SMILES: CCOc1ccc2nc(-c3ccnnc3)nc(Nc3cccc(OC(F)F)c3)c2c1] |
| 1168 | 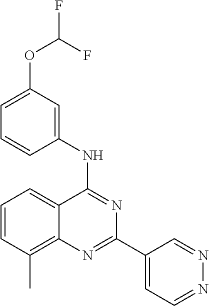 [SMILES: Cc1cccc2c(Nc3cccc(OC(F)F)c3)nc(-c3ccnnc3)nc12] |
| 1169 | 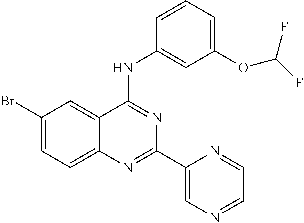 [SMILES: FC(F)Oc1cccc(Nc2nc(-c3cnccn3)nc3ccc(Br)cc23)c1] |
| 1170 | 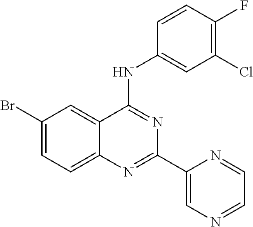 [SMILES: Fc1ccc(Nc2nc(-c3cnccn3)nc3ccc(Br)cc23)cc1Cl] |
| 1171 | 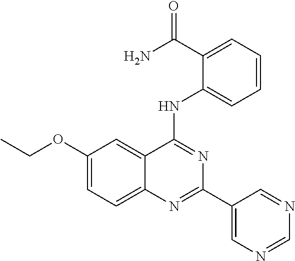 [SMILES: CCOc1ccc2nc(-c3cncnc3)nc(Nc3ccccc3C(N)=O)c2c1] |
| 1172 | 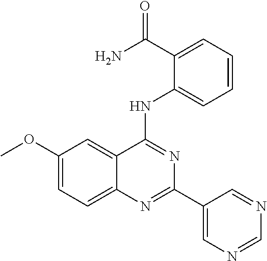 [SMILES: COc1ccc2nc(-c3cncnc3)nc(Nc3ccccc3C(N)=O)c2c1] |
| 1173 | 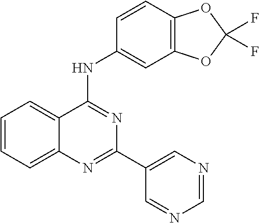 [SMILES: FC1(F)Oc2ccc(Nc3nc(-c4cncnc4)nc4ccccc34)cc2O1] |
| 1174 | 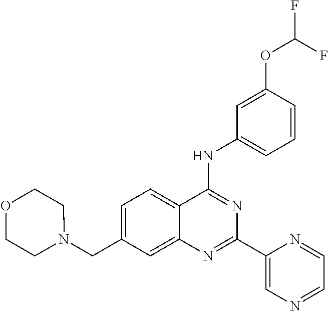 [SMILES: FC(F)Oc1cccc(Nc2nc(-c3cnccn3)nc3cc(CN4CCOCC4)ccc23)c1] |
| 1175 | 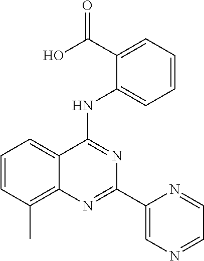 [SMILES: Cc1cccc2c(Nc3ccccc3C(=O)O)nc(-c3cnccn3)nc12] |
| 1176 | 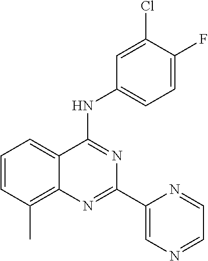 [SMILES: Cc1cccc2c(Nc3ccc(F)c(Cl)c3)nc(-c3cnccn3)nc12] |
| 1177 | 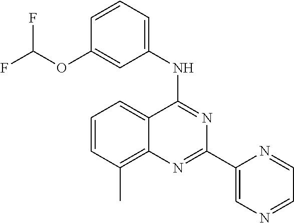 [SMILES: Cc1cccc2c(Nc3cccc(OC(F)F)c3)nc(-c3cnccn3)nc12] |
| 1178 | 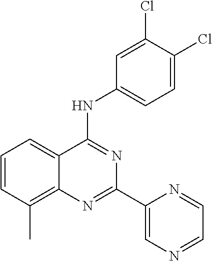 [SMILES: Cc1cccc2c(Nc3ccc(Cl)c(Cl)c3)nc(-c3cnccn3)nc12] |
| 1179 | 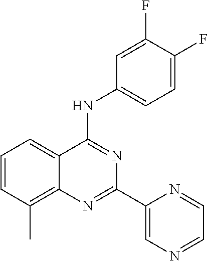 [SMILES: Cc1cccc2c(Nc3ccc(F)c(F)c3)nc(-c3cnccn3)nc12] |
| 1180 | 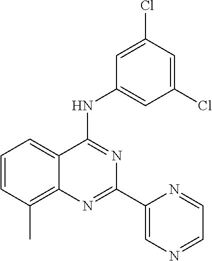 [SMILES: Cc1cccc2c(Nc3cc(Cl)cc(Cl)c3)nc(-c3cnccn3)nc12] |
| 1181 | 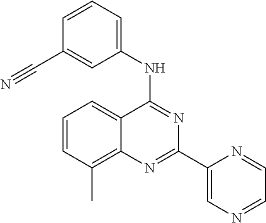 [SMILES: Cc1cccc2c(Nc3cccc(C#N)c3)nc(-c3cnccn3)nc12] |
| 1182 | 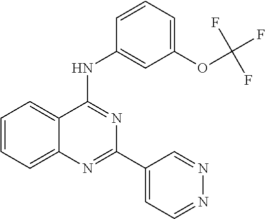 [SMILES: FC(F)(F)Oc1cccc(Nc2nc(-c3ccnnc3)nc3ccccc23)c1] |
| 1183 | 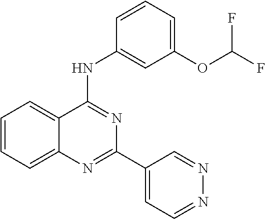 [SMILES: FC(F)Oc1cccc(Nc2nc(-c3ccnnc3)nc3ccccc23)c1] |
| 1184 | 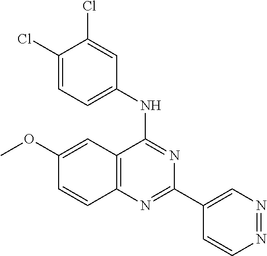 [SMILES: COc1ccc2nc(-c3ccnnc3)nc(Nc3ccc(Cl)c(Cl)c3)c2c1] |
| 1185 | 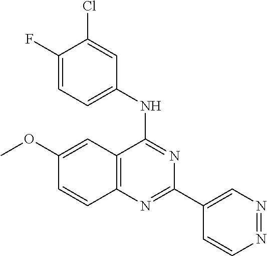 [SMILES: COc1ccc2nc(-c3ccnnc3)nc(Nc3ccc(F)c(Cl)c3)c2c1] |
| 1186 | 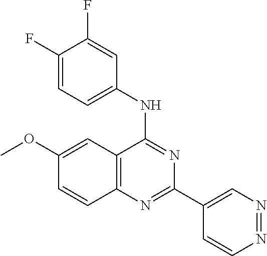 [SMILES: COc1ccc2nc(-c3ccnnc3)nc(Nc3ccc(F)c(F)c3)c2c1] |
| 1187 | 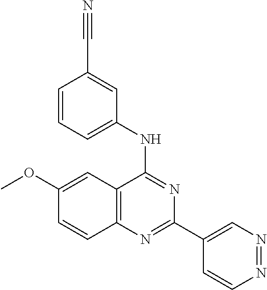 [SMILES: COc1ccc2nc(-c3ccnnc3)nc(Nc3cccc(C#N)c3)c2c1] |
| 1188 | 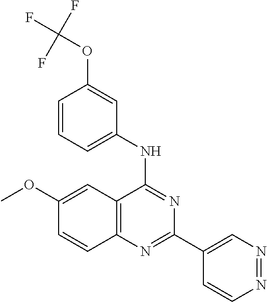 [SMILES: COc1ccc2nc(-c3ccnnc3)nc(Nc3cccc(OC(F)(F)F)c3)c2c1] |
| 1189 | 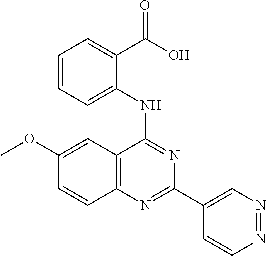 [SMILES: COc1ccc2nc(-c3ccnnc3)nc(Nc3ccccc3C(=O)O)c2c1] |
| 1190 | 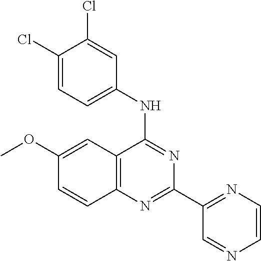 [SMILES: COc1ccc2nc(-c3cnccn3)nc(Nc3ccc(Cl)c(Cl)c3)c2c1] |
| 1191 | 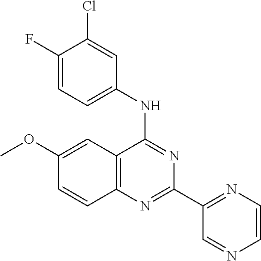 [SMILES: COc1ccc2nc(-c3cnccn3)nc(Nc3ccc(F)c(Cl)c3)c2c1] |
| 1192 | 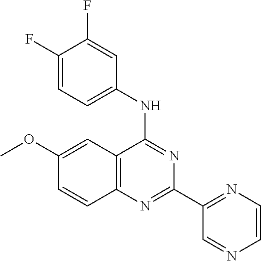 [SMILES: COc1ccc2nc(-c3cnccn3)nc(Nc3ccc(F)c(F)c3)c2c1] |
| 1193 | 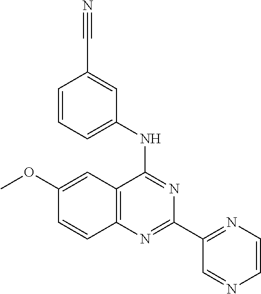 [SMILES: COc1ccc2nc(-c3cnccn3)nc(Nc3cccc(C#N)c3)c2c1] |
| 1194 | 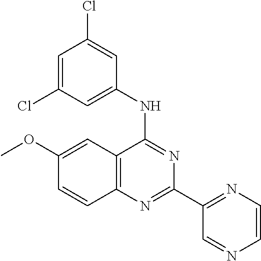 [SMILES: COc1ccc2nc(-c3cnccn3)nc(Nc3cc(Cl)cc(Cl)c3)c2c1] |
| 1195 | 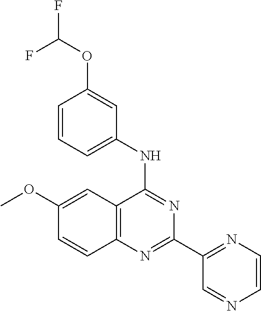 [SMILES: COc1ccc2nc(-c3cnccn3)nc(Nc3cccc(OC(F)F)c3)c2c1] |
| 1196 | 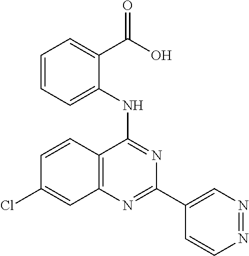 [SMILES: O=C(O)c1ccccc1Nc1nc(-c2ccnnc2)nc2cc(Cl)ccc12] |
| 1197 | 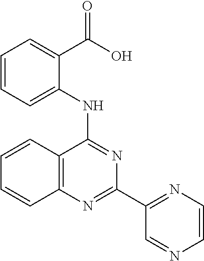 [SMILES: O=C(O)c1ccccc1Nc1nc(-c2cnccn2)nc2ccccc12] |
| 1198 | 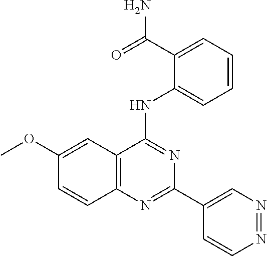 [SMILES: COc1ccc2nc(-c3ccnnc3)nc(Nc3ccccc3C(N)=O)c2c1] |
| 1199 | 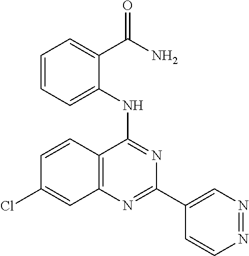 [SMILES: NC(=O)c1ccccc1Nc1nc(-c2ccnnc2)nc2cc(Cl)ccc12] |
| 1200 | 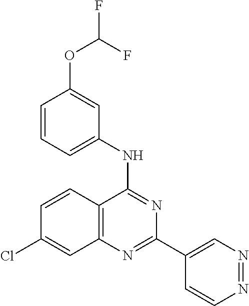 [SMILES: FC(F)Oc1cccc(Nc2nc(-c3ccnnc3)nc3cc(Cl)ccc23)c1] |
| 1201 | 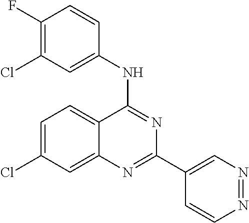 [SMILES: Fc1ccc(Nc2nc(-c3ccnnc3)nc3cc(Cl)ccc23)cc1Cl] |
| 1202 | 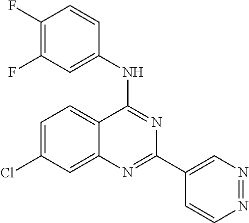 [SMILES: Fc1ccc(Nc2nc(-c3ccnnc3)nc3cc(Cl)ccc23)cc1F] |
| 1203 | 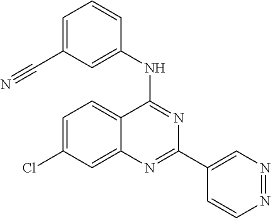 [SMILES: N#Cc1cccc(Nc2nc(-c3ccnnc3)nc3cc(Cl)ccc23)c1] |
| 1204 | 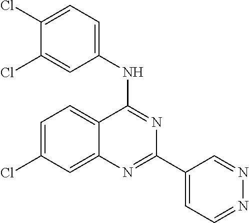 [SMILES: Clc1ccc2c(Nc3ccc(Cl)c(Cl)c3)nc(-c3ccnnc3)nc2c1] |
| 1205 | 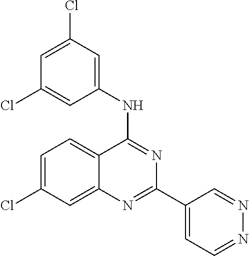 [SMILES: Clc1cc(Cl)cc(Nc2nc(-c3ccnnc3)nc3cc(Cl)ccc23)c1] |
| 1206 | 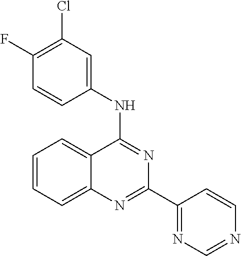 [SMILES: Fc1ccc(Nc2nc(-c3ccncn3)nc3ccccc23)cc1Cl] |
| 1207 | 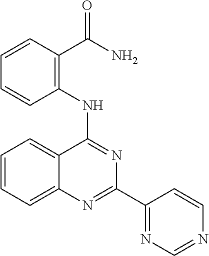 [SMILES: NC(=O)c1ccccc1Nc1nc(-c2ccncn2)nc2ccccc12] |
| 1208 | 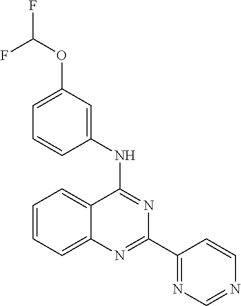 [SMILES: FC(F)Oc1cccc(Nc2nc(-c3ccncn3)nc3ccccc23)c1] |
| 1209 | 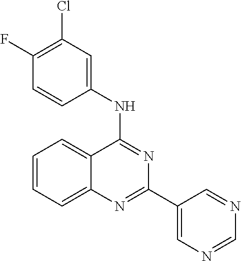 [SMILES: Fc1ccc(Nc2nc(-c3cncnc3)nc3ccccc23)cc1Cl] |
| 1210 | 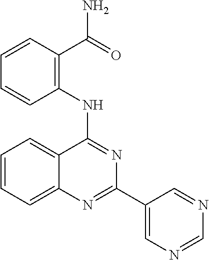 [SMILES: NC(=O)c1ccccc1Nc1nc(-c2cncnc2)nc2ccccc12] |
| 1211 | 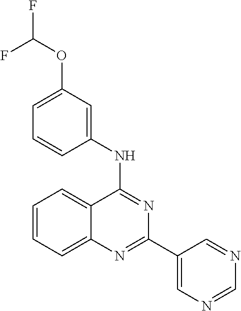 [SMILES: FC(F)Oc1cccc(Nc2nc(-c3cncnc3)nc3ccccc23)c1] |
| 1212 | 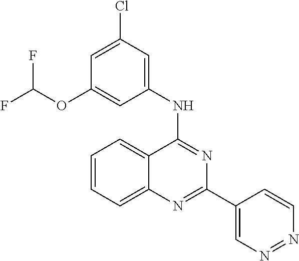 [SMILES: FC(F)Oc1cc(Cl)cc(Nc2nc(-c3ccnnc3)nc3ccccc23)c1] |
| 1213 | 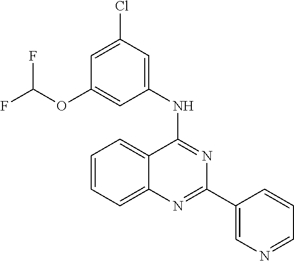 [SMILES: FC(F)Oc1cc(Cl)cc(Nc2nc(-c3cccnc3)nc3ccccc23)c1] |
| 1214 | 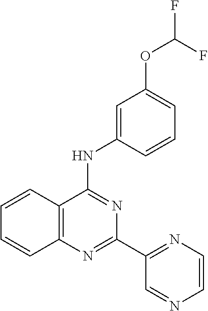 [SMILES: FC(F)Oc1cccc(Nc2nc(-c3cnccn3)nc3ccccc23)c1] |
| 1215 | 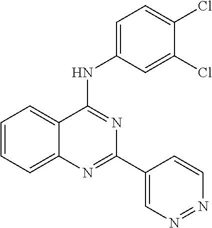 [SMILES: Clc1ccc(Nc2nc(-c3ccnnc3)nc3ccccc23)cc1Cl] |
| 1216 | 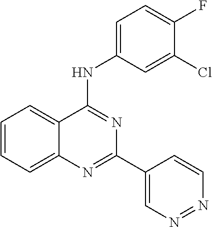 [SMILES: Fc1ccc(Nc2nc(-c3ccnnc3)nc3ccccc23)cc1Cl] |
| 1217 | 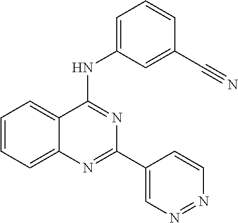 [SMILES: N#Cc1cccc(Nc2nc(-c3ccnnc3)nc3ccccc23)c1] |
| 1218 | 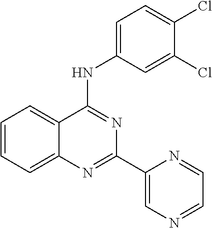 [SMILES: Clc1ccc(Nc2nc(-c3cnccn3)nc3ccccc23)cc1Cl] |
| 1219 | 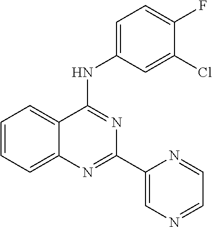 [SMILES: Fc1ccc(Nc2nc(-c3cnccn3)nc3ccccc23)cc1Cl] |
| 1220 | 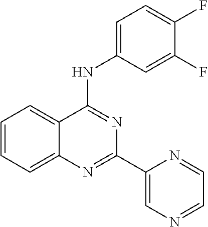 [SMILES: Fc1ccc(Nc2nc(-c3cnccn3)nc3ccccc23)cc1F] |
| 1221 | 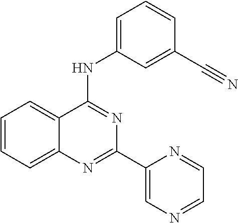 [SMILES: N#Cc1cccc(Nc2nc(-c3cnccn3)nc3ccccc23)c1] |
| 1222 | 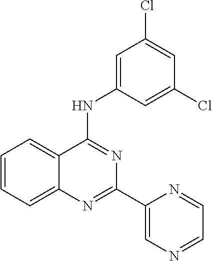 [SMILES: Clc1cc(Cl)cc(Nc2nc(-c3cnccn3)nc3ccccc23)c1] |
| 1223 | 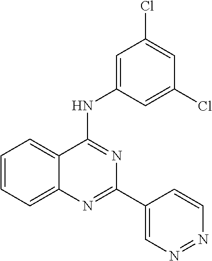 [SMILES: Clc1cc(Cl)cc(Nc2nc(-c3ccnnc3)nc3ccccc23)c1] |
| 1224 | 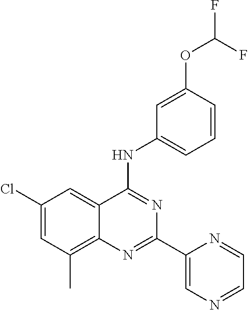 [SMILES: Cc1cc(Cl)cc2c(Nc3cccc(OC(F)F)c3)nc(-c3cnccn3)nc12] |
| 1225 | 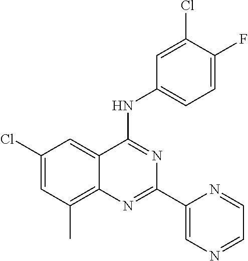 [SMILES: Cc1cc(Cl)cc2c(Nc3ccc(F)c(Cl)c3)nc(-c3cnccn3)nc12] |
| 1226 | 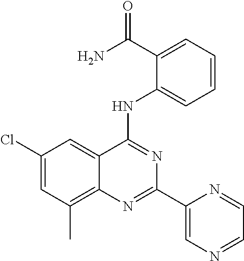 [SMILES: Cc1cc(Cl)cc2c(Nc3ccccc3C(N)=O)nc(-c3cnccn3)nc12] |
| 1227 | 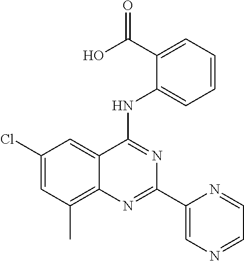 [SMILES: Cc1cc(Cl)cc2c(Nc3ccccc3C(=O)O)nc(-c3cnccn3)nc12] |
| 1228 | 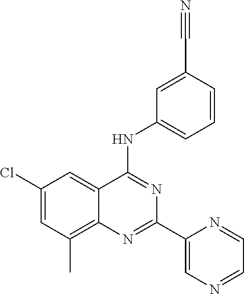 [SMILES: Cc1cc(Cl)cc2c(Nc3cccc(C#N)c3)nc(-c3cnccn3)nc12] |
| 1229 | 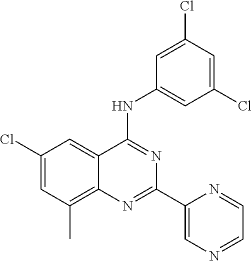 [SMILES: Cc1cc(Cl)cc2c(Nc3cc(Cl)cc(Cl)c3)nc(-c3cnccn3)nc12] |
| 1230 | 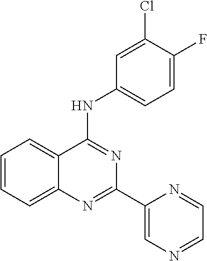 [SMILES: Fc1ccc(Nc2nc(-c3cnccn3)nc3ccccc23)cc1Cl] |
| 1231 | 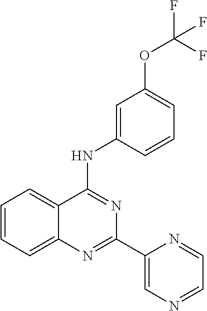 [SMILES: FC(F)(F)Oc1cccc(Nc2nc(-c3cnccn3)nc3ccccc23)c1] |
| 1232 | 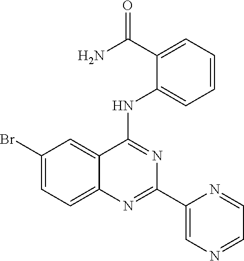 [SMILES: NC(=O)c1ccccc1Nc1nc(-c2cnccn2)nc2ccc(Br)cc12] |
| 1233 | 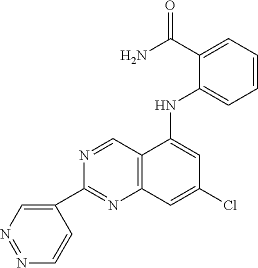 [SMILES: NC(=O)c1ccccc1Nc1cc(Cl)cc2nc(-c3ccnnc3)ncc12] |
| 1234 | 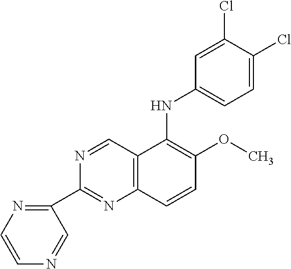 [SMILES: COc1ccc2nc(-c3cnccn3)ncc2c1Nc1ccc(Cl)c(Cl)c1] |
| 1235 | 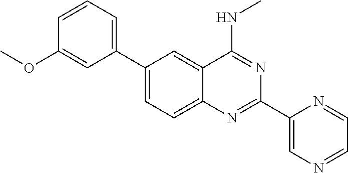 [SMILES: CNc1nc(-c2cnccn2)nc2ccc(-c3cccc(OC)c3)cc12] |
| 1236 | 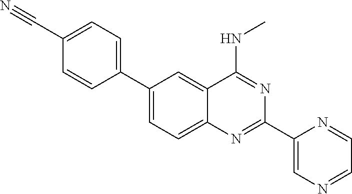 [SMILES: CNc1nc(-c2cnccn2)nc2ccc(-c3ccc(C#N)cc3)cc12] |
| 1237 | 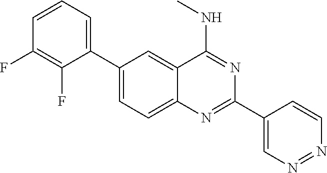 [SMILES: CNc1nc(-c2ccnnc2)nc2ccc(-c3cccc(F)c3F)cc12] |
| 1238 | 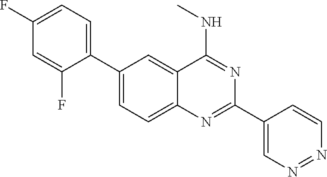 [SMILES: CNc1nc(-c2ccnnc2)nc2ccc(-c3ccc(F)cc3F)cc12] |
| 1239 | 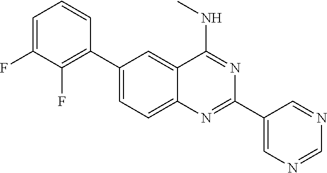 [SMILES: CNc1nc(-c2cncnc2)nc2ccc(-c3cccc(F)c3F)cc12] |
| 1240 | 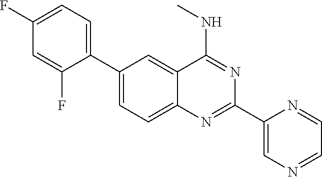 [SMILES: CNc1nc(-c2cnccn2)nc2ccc(-c3ccc(F)cc3F)cc12] |
| 1241 | 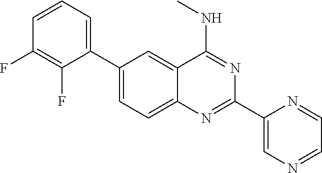 [SMILES: CNc1nc(-c2cnccn2)nc2ccc(-c3cccc(F)c3F)cc12] |
| 1242 | 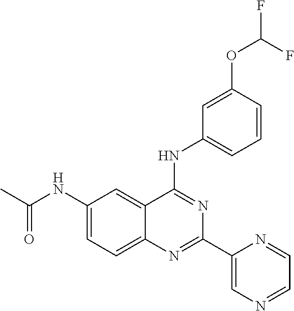 [SMILES: CC(=O)Nc1ccc2nc(-c3cnccn3)nc(Nc3cccc(OC(F)F)c3)c2c1] |
| 1243 | 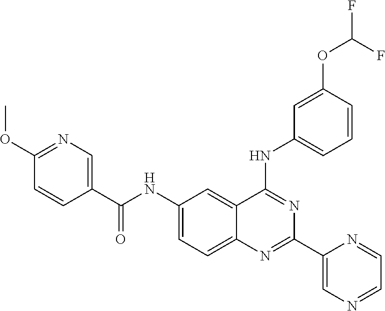 [SMILES: COc1ccc(C(=O)Nc2ccc3nc(-c4cnccn4)nc(Nc4cccc(OC(F)F)c4)c3c2)cn1] |
| 1244 | 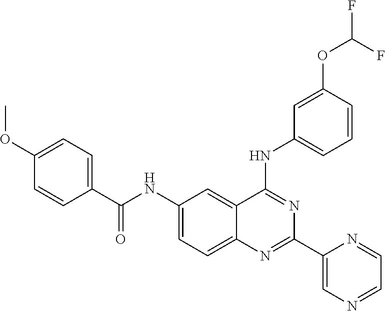 [SMILES: COc1ccc(C(=O)Nc2ccc3nc(-c4cnccn4)nc(Nc4cccc(OC(F)F)c4)c3c2)cc1] |
| 1245 | 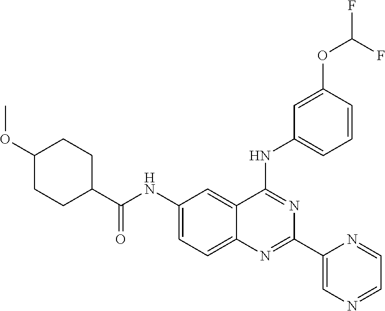 [SMILES: COC1CCC(C(=O)Nc2ccc3nc(-c4cnccn4)nc(Nc4cccc(OC(F)F)c4)c3c2)CC1] |
| 1246 | 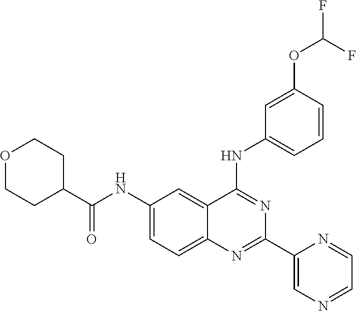 [SMILES: O=C(Nc1ccc2nc(-c3cnccn3)nc(Nc3cccc(OC(F)F)c3)c2c1)C1CCOCC1] |
| 1247 | 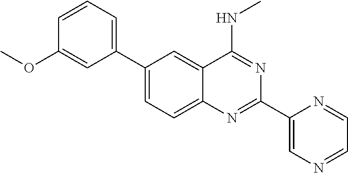 [SMILES: CNc1nc(-c2cnccn2)nc2ccc(-c3cccc(OC)c3)cc12] |
| 1248 | 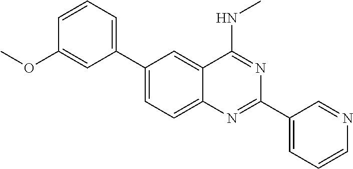 [SMILES: CNc1nc(-c2cccnc2)nc2ccc(-c3cccc(OC)c3)cc12] |
| 1249 | 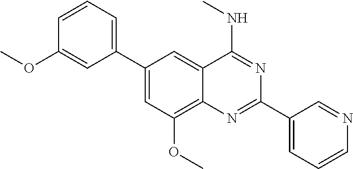 [SMILES: CNc1nc(-c2cccnc2)nc2c(OC)cc(-c3cccc(OC)c3)cc12] |
| 1250 | 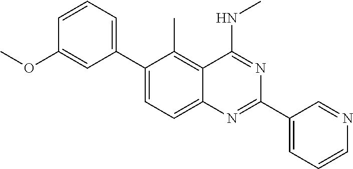 [SMILES: CNc1nc(-c2cccnc2)nc2ccc(-c3cccc(OC)c3)c(C)c12] |
| 1251 | 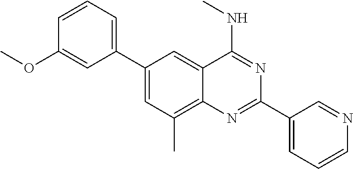 [SMILES: CNc1nc(-c2cccnc2)nc2c(C)cc(-c3cccc(OC)c3)cc12] |
| 1252 | 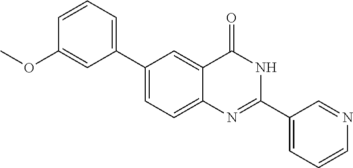 [SMILES: COc1cccc(-c2ccc3nc(-c4cccnc4)[nH]c(=O)c3c2)c1] |
| 1253 | 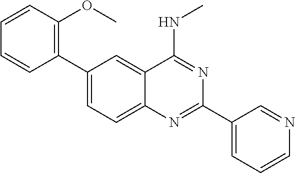 [SMILES: CNc1nc(-c2cccnc2)nc2ccc(-c3ccccc3OC)cc12] |
| 1254 | 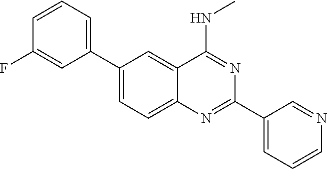 [SMILES: CNc1nc(-c2cccnc2)nc2ccc(-c3cccc(F)c3)cc12] |
| 1255 | 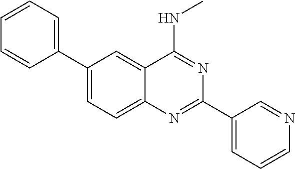 [SMILES: CNc1nc(-c2cccnc2)nc2ccc(-c3ccccc3)cc12] |
| 1256 | 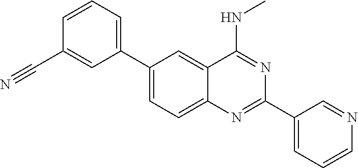 [SMILES: CNc1nc(-c2cccnc2)nc2ccc(-c3cccc(C#N)c3)cc12] |
| 1257 | 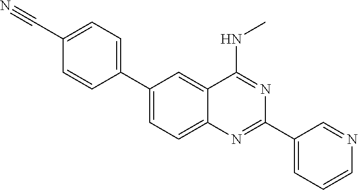 [SMILES: CNc1nc(-c2cccnc2)nc2ccc(-c3ccc(C#N)cc3)cc12] |
| 1258 | 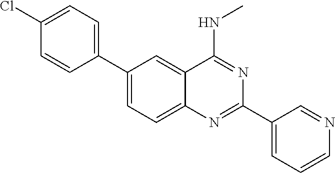 [SMILES: CNc1nc(-c2cccnc2)nc2ccc(-c3ccc(Cl)cc3)cc12] |
| 1259 | 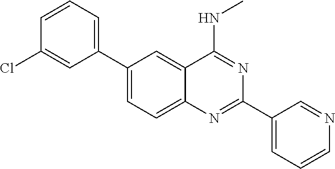 [SMILES: CNc1nc(-c2cccnc2)nc2ccc(-c3cccc(Cl)c3)cc12] |
| 1260 | 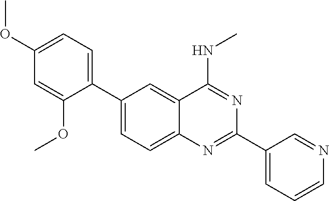 [SMILES: CNc1nc(-c2cccnc2)nc2ccc(-c3ccc(OC)cc3OC)cc12] |
| 1261 | 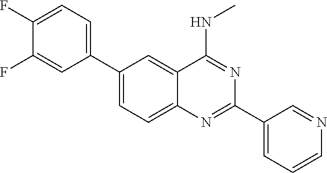 [SMILES: CNc1nc(-c2cccnc2)nc2ccc(-c3ccc(F)c(F)c3)cc12] |
| 1262 | 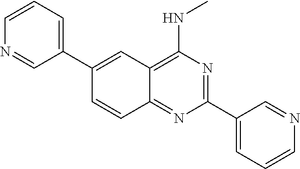 [SMILES: CNc1nc(-c2cccnc2)nc2ccc(-c3cccnc3)cc12] |
| 1263 | 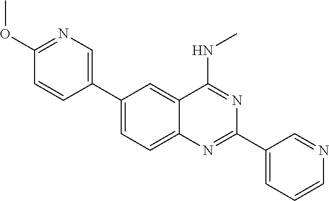 [SMILES: CNc1nc(-c2cccnc2)nc2ccc(-c3ccc(OC)nc3)cc12] |
| 1264 | 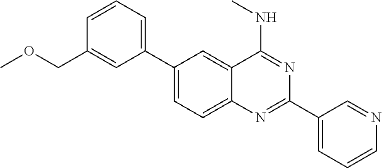 [SMILES: CNc1nc(-c2cccnc2)nc2ccc(-c3cccc(COC)c3)cc12] |
| 1265 | 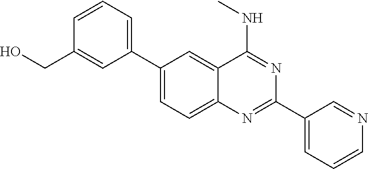 [SMILES: CNc1nc(-c2cccnc2)nc2ccc(-c3cccc(CO)c3)cc12] |
| 1266 | 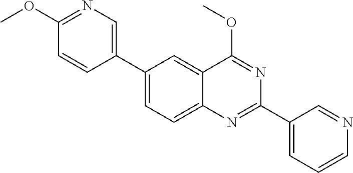 [SMILES: COc1ccc(-c2ccc3nc(-c4cccnc4)nc(OC)c3c2)cn1] |
| 1267 | 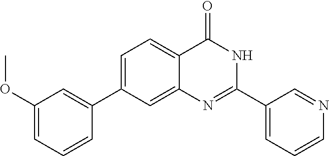 [SMILES: COc1cccc(-c2ccc3c(=O)[nH]c(-c4cccnc4)nc3c2)c1] |
| 1268 | 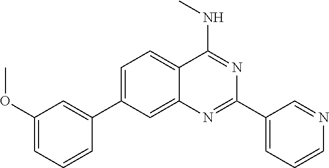 [SMILES: CNc1nc(-c2cccnc2)nc2cc(-c3cccc(OC)c3)ccc12] |
| 1269 | 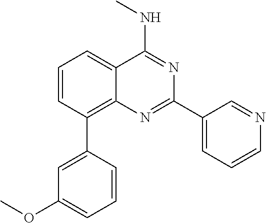 [SMILES: CNc1nc(-c2cccnc2)nc2c(-c3cccc(OC)c3)cccc12] |
| 1270 | 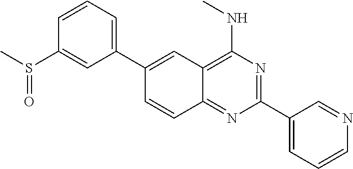 [SMILES: CNc1nc(-c2cccnc2)nc2ccc(-c3cccc(S(C)=O)c3)cc12] |
| 1271 | 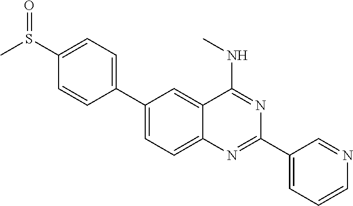 [SMILES: CNc1nc(-c2cccnc2)nc2ccc(-c3ccc(S(C)=O)cc3)cc12] |
| 1272 | 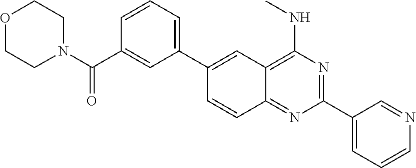 [SMILES: CNc1nc(-c2cccnc2)nc2ccc(-c3cccc(C(=O)N4CCOCC4)c3)cc12] |
| 1273 | 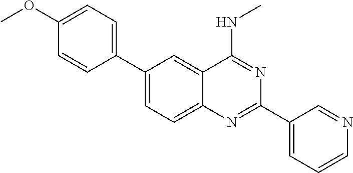 [SMILES: CNc1nc(-c2cccnc2)nc2ccc(-c3ccc(OC)cc3)cc12] |
| 1274 | 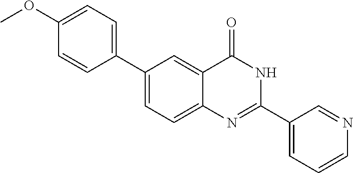 [SMILES: COc1ccc(-c2ccc3nc(-c4cccnc4)[nH]c(=O)c3c2)cc1] |
| 1275 | 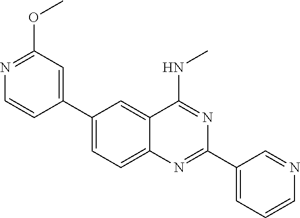 [SMILES: CNc1nc(-c2cccnc2)nc2ccc(-c3ccnc(OC)c3)cc12] |
| 1276 | 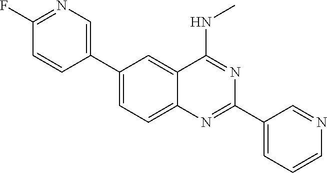 [SMILES: CNc1nc(-c2cccnc2)nc2ccc(-c3ccc(F)nc3)cc12] |
| 1277 | 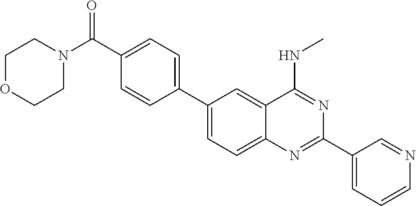 [SMILES: CNc1nc(-c2cccnc2)nc2ccc(-c3ccc(C(=O)N4CCOCC4)cc3)cc12] |
| 1278 | 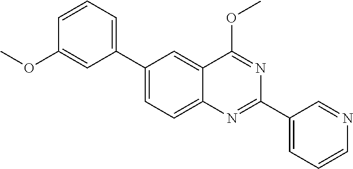 [SMILES: COc1cccc(-c2ccc3nc(-c4cccnc4)nc(OC)c3c2)c1] |
| 1279 | 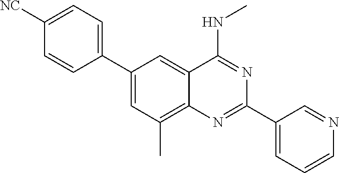 [SMILES: CNc1nc(-c2cccnc2)nc2c(C)cc(-c3ccc(C#N)cc3)cc12] |
| 1280 | 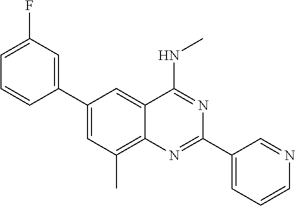 [SMILES: CNc1nc(-c2cccnc2)nc2c(C)cc(-c3cccc(F)c3)cc12] |
| 1281 | 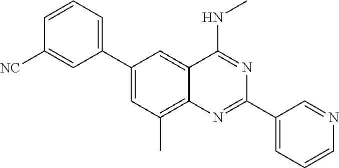 [SMILES: CNc1nc(-c2cccnc2)nc2c(C)cc(-c3cccc(C#N)c3)cc12] |
| 1282 | 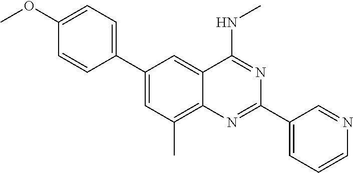 [SMILES: CNc1nc(-c2cccnc2)nc2c(C)cc(-c3ccc(OC)cc3)cc12] |
| 1283 | 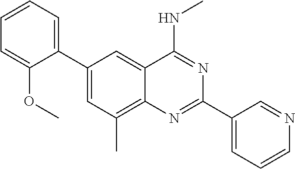 [SMILES: CNc1nc(-c2cccnc2)nc2c(C)cc(-c3ccccc3OC)cc12] |
| 1284 | 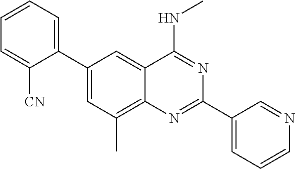 [SMILES: CNc1nc(-c2cccnc2)nc2c(C)cc(-c3ccccc3C#N)cc12] |
| 1285 | 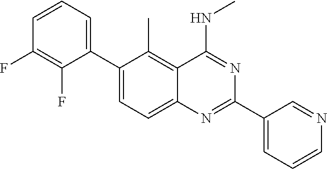 [SMILES: CNc1nc(-c2cccnc2)nc2ccc(-c3cccc(F)c3F)c(C)c12] |
| 1286 | 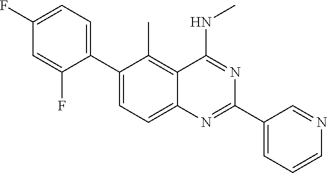 [SMILES: CNc1nc(-c2cccnc2)nc2ccc(-c3ccc(F)cc3F)c(C)c12] |
| 1287 | 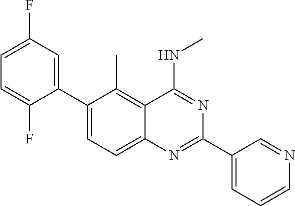 [SMILES: CNc1nc(-c2cccnc2)nc2ccc(-c3cc(F)ccc3F)c(C)c12] |
| 1288 | 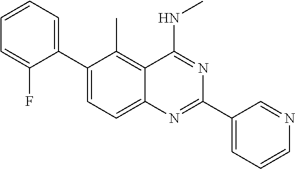 [SMILES: CNc1nc(-c2cccnc2)nc2ccc(-c3ccccc3F)c(C)c12] |
| 1289 | 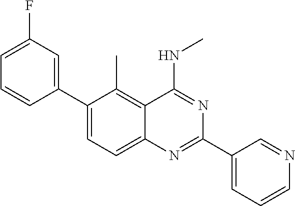 [SMILES: CNc1nc(-c2cccnc2)nc2ccc(-c3cccc(F)c3)c(C)c12] |
| 1290 | 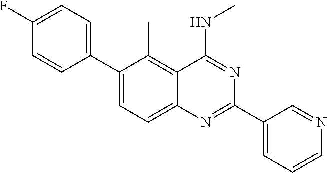 [SMILES: CNc1nc(-c2cccnc2)nc2ccc(-c3ccc(F)cc3)c(C)c12] |
| 1291 | 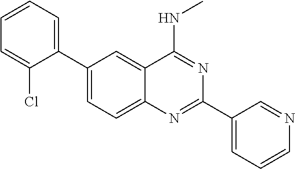 [SMILES: CNc1nc(-c2cccnc2)nc2ccc(-c3ccccc3Cl)cc12] |
| 1292 | 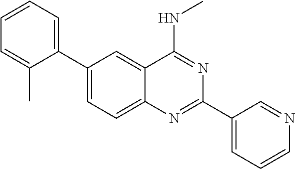 [SMILES: CNc1nc(-c2cccnc2)nc2ccc(-c3ccccc3C)cc12] |
| 1293 | 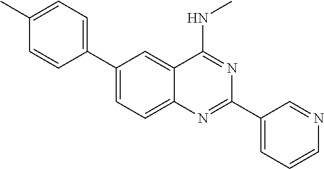 [SMILES: CNc1nc(-c2cccnc2)nc2ccc(-c3ccc(C)cc3)cc12] |
| 1294 | 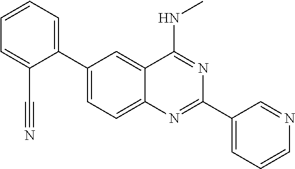 [SMILES: CNc1nc(-c2cccnc2)nc2ccc(-c3ccccc3C#N)cc12] |
| 1295 | 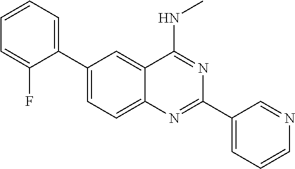 [SMILES: CNc1nc(-c2cccnc2)nc2ccc(-c3ccccc3F)cc12] |
| 1296 | 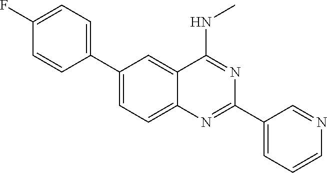 [SMILES: CNc1nc(-c2cccnc2)nc2ccc(-c3ccc(F)cc3)cc12] |
| 1297 | 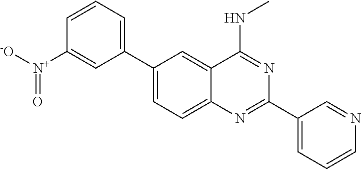 |
| 1298 | 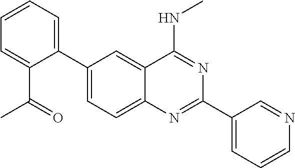 [SMILES: CNc1nc(-c2cccnc2)nc2ccc(-c3ccccc3C(C)=O)cc12] |
| 1299 | 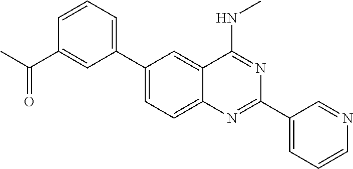 [SMILES: CNc1nc(-c2cccnc2)nc2ccc(-c3cccc(C(C)=O)c3)cc12] |
| 1300 | 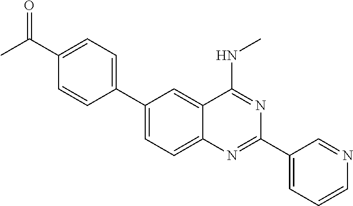 [SMILES: CNc1nc(-c2cccnc2)nc2ccc(-c3ccc(C(C)=O)cc3)cc12] |
| 1301 | 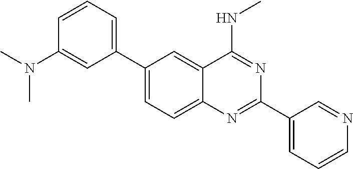 [SMILES: CNc1nc(-c2cccnc2)nc2ccc(-c3cccc(N(C)C)c3)cc12] |
| 1302 | 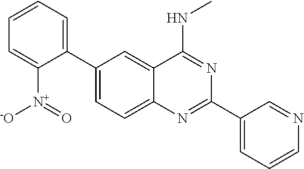 |
| 1303 | 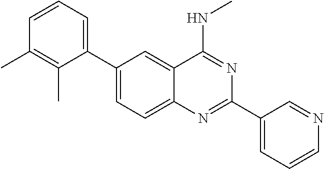 [SMILES: CNc1nc(-c2cccnc2)nc2ccc(-c3cccc(C)c3C)cc12] |
| 1304 | 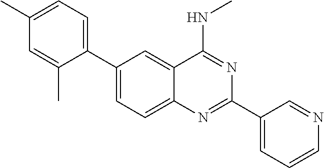 [SMILES: CNc1nc(-c2cccnc2)nc2ccc(-c3ccc(C)cc3C)cc12] |
| 1305 | 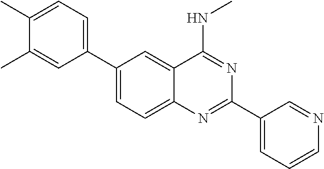 [SMILES: CNc1nc(-c2cccnc2)nc2ccc(-c3ccc(C)c(C)c3)cc12] |
| 1306 | 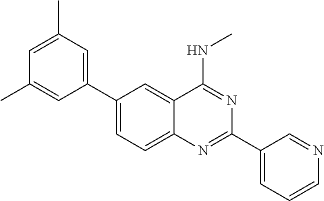 [SMILES: CNc1nc(-c2cccnc2)nc2ccc(-c3cc(C)cc(C)c3)cc12] |
| 1307 | 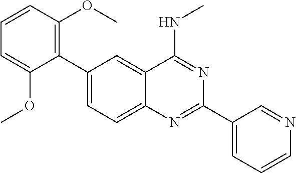 [SMILES: CNc1nc(-c2cccnc2)nc2ccc(-c3c(OC)cccc3OC)cc12] |
| 1308 | 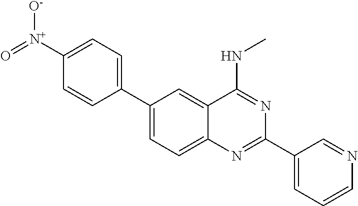 [SMILES: CNc1nc(-c2cccnc2)nc2ccc(-c3ccc([N+](=O)[O-])cc3)cc12] |
| 1309 | 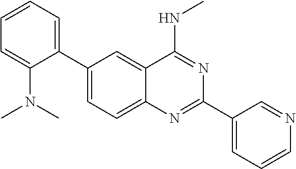 [SMILES: CNc1nc(-c2cccnc2)nc2ccc(-c3ccccc3N(C)C)cc12] |
| 1310 | 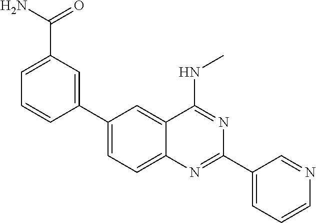 [SMILES: CNc1nc(-c2cccnc2)nc2ccc(-c3cccc(C(N)=O)c3)cc12] |
| 1311 | 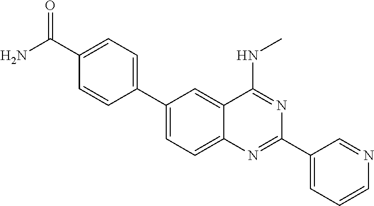 [SMILES: CNc1nc(-c2cccnc2)nc2ccc(-c3ccc(C(N)=O)cc3)cc12] |
| 1312 | 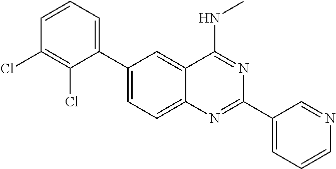 [SMILES: CNc1nc(-c2cccnc2)nc2ccc(-c3cccc(Cl)c3Cl)cc12] |
| 1313 | 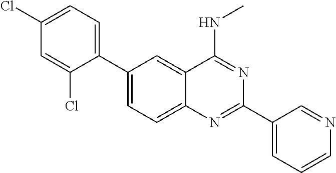 [SMILES: CNc1nc(-c2cccnc2)nc2ccc(-c3ccc(Cl)cc3Cl)cc12] |
| 1314 | 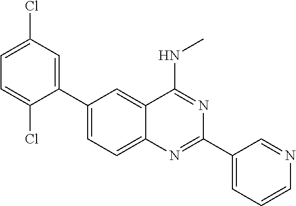 [SMILES: CNc1nc(-c2cccnc2)nc2ccc(-c3cc(Cl)ccc3Cl)cc12] |
| 1315 | 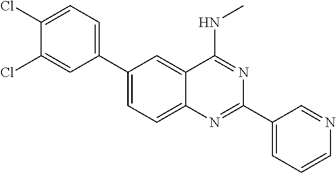 [SMILES: CNc1nc(-c2cccnc2)nc2ccc(-c3ccc(Cl)c(Cl)c3)cc12] |
| 1316 | 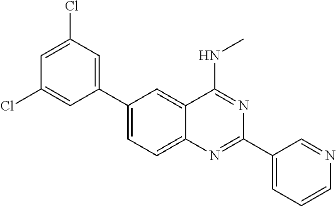 [SMILES: CNc1nc(-c2cccnc2)nc2ccc(-c3cc(Cl)cc(Cl)c3)cc12] |
| 1317 | 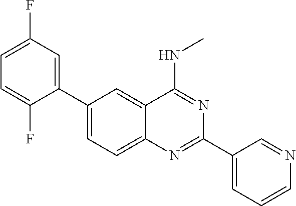 [SMILES: CNc1nc(-c2cccnc2)nc2ccc(-c3cc(F)ccc3F)cc12] |
| 1318 | 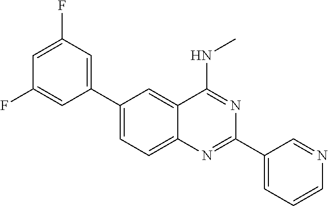 [SMILES: CNc1nc(-c2cccnc2)nc2ccc(-c3cc(F)cc(F)c3)cc12] |
| 1319 | 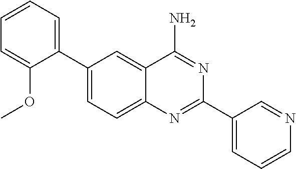 [SMILES: COc1ccccc1-c1ccc2nc(-c3cccnc3)nc(N)c2c1] |
| 1320 | 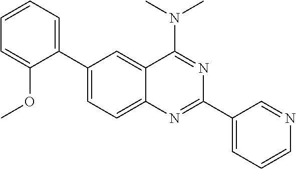 [SMILES: COc1ccccc1-c1ccc2nc(-c3cccnc3)nc(N(C)C)c2c1] |
| 1321 | 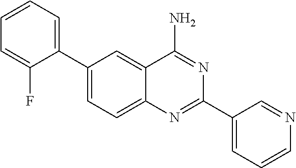 [SMILES: Nc1nc(-c2cccnc2)nc2ccc(-c3ccccc3F)cc12] |
| 1322 | 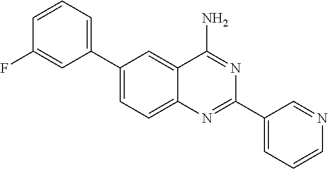 [SMILES: Nc1nc(-c2cccnc2)nc2ccc(-c3cccc(F)c3)cc12] |
| 1323 | 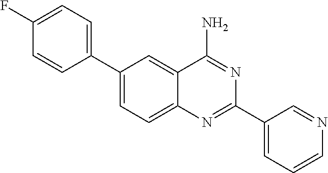 [SMILES: Nc1nc(-c2cccnc2)nc2ccc(-c3ccc(F)cc3)cc12] |
| 1324 | 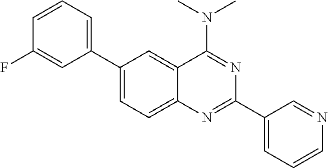 [SMILES: CN(C)c1nc(-c2cccnc2)nc2ccc(-c3cccc(F)c3)cc12] |
| 1325 | 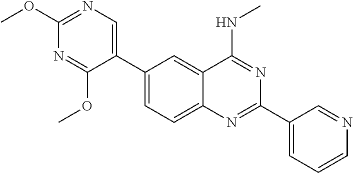 [SMILES: CNc1nc(-c2cccnc2)nc2ccc(-c3cnc(OC)nc3OC)cc12] |
| 1326 | 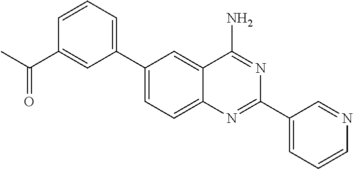 [SMILES: CC(=O)c1cccc(-c2ccc3nc(-c4cccnc4)nc(N)c3c2)c1] |
| 1327 | 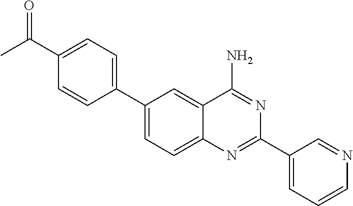 [SMILES: CC(=O)c1ccc(-c2ccc3nc(-c4cccnc4)nc(N)c3c2)cc1] |
| 1328 | 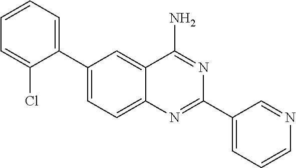 [SMILES: Nc1nc(-c2cccnc2)nc2ccc(-c3ccccc3Cl)cc12] |
| 1329 | 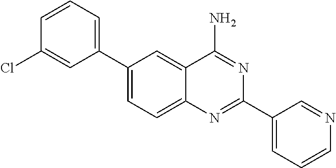 [SMILES: Nc1nc(-c2cccnc2)nc2ccc(-c3cccc(Cl)c3)cc12] |
| 1330 | 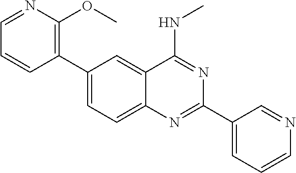 [SMILES: CNc1nc(-c2cccnc2)nc2ccc(-c3cccnc3OC)cc12] |
| 1331 | 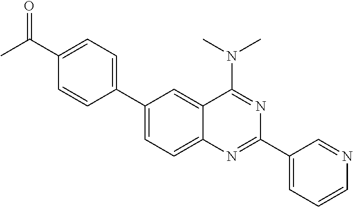 [SMILES: CC(=O)c1ccc(-c2ccc3nc(-c4cccnc4)nc(N(C)C)c3c2)cc1] |
| 1332 | 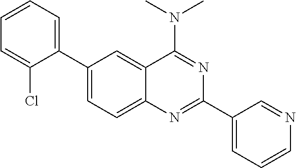 [SMILES: CN(C)c1nc(-c2cccnc2)nc2ccc(-c3ccccc3Cl)cc12] |
| 1333 | 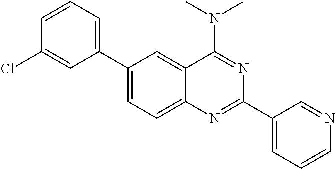 [SMILES: CN(C)c1nc(-c2cccnc2)nc2ccc(-c3cccc(Cl)c3)cc12] |
| 1334 | 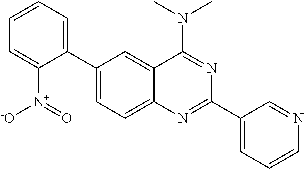 |
| 1335 | 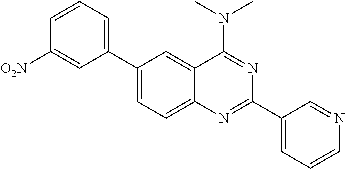 [SMILES: CN(C)c1nc(-c2cccnc2)nc2ccc(-c3cccc([N+](=O)[O-])c3)cc12] |
| 1336 | 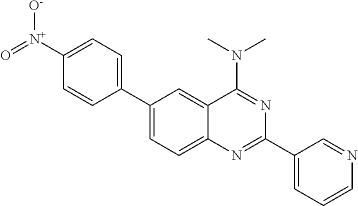 [SMILES: CN(C)c1nc(-c2cccnc2)nc2ccc(-c3ccc([N+](=O)[O-])cc3)cc12] |
| 1337 | 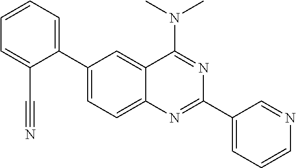 [SMILES: CN(C)c1nc(-c2cccnc2)nc2ccc(-c3ccccc3C#N)cc12] |
| 1338 | 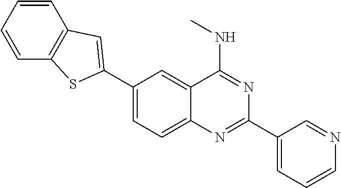 [SMILES: CNc1nc(-c2cccnc2)nc2ccc(-c3cc4ccccc4s3)cc12] |
| 1339 | 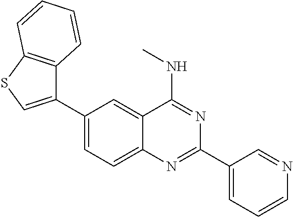 [SMILES: CNc1nc(-c2cccnc2)nc2ccc(-c3csc4ccccc34)cc12] |
| 1340 | 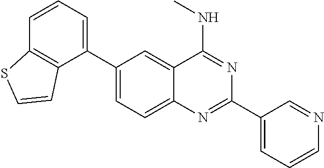 [SMILES: CNc1nc(-c2cccnc2)nc2ccc(-c3cccc4sccc34)cc12] |
| 1341 | 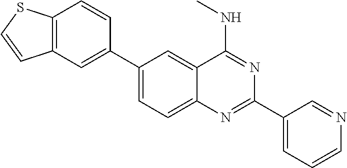 [SMILES: CNc1nc(-c2cccnc2)nc2ccc(-c3ccc4sccc4c3)cc12] |
| 1342 | 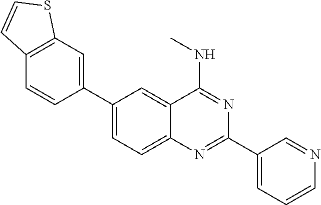 [SMILES: CNc1nc(-c2cccnc2)nc2ccc(-c3ccc4ccsc4c3)cc12] |
| 1343 | 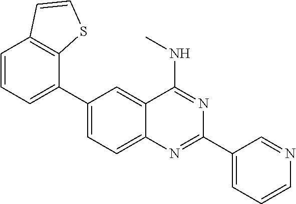 [SMILES: CNc1nc(-c2cccnc2)nc2ccc(-c3cccc4ccsc34)cc12] |
| 1344 | 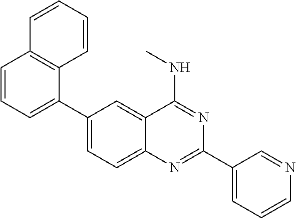 [SMILES: CNc1nc(-c2cccnc2)nc2ccc(-c3cccc4ccccc34)cc12] |
| 1345 | 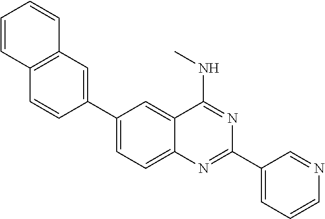 [SMILES: CNc1nc(-c2cccnc2)nc2ccc(-c3ccc4ccccc4c3)cc12] |
| 1346 | 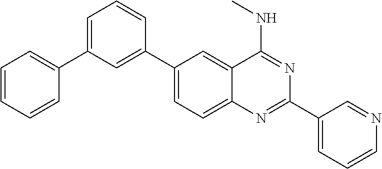 [SMILES: CNc1nc(-c2cccnc2)nc2ccc(-c3cccc(-c4ccccc4)c3)cc12] |
| 1347 | 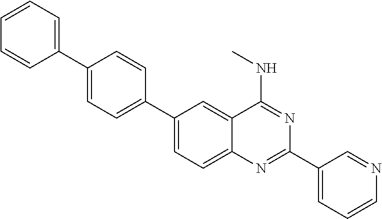 [SMILES: CNc1nc(-c2cccnc2)nc2ccc(-c3ccc(-c4ccccc4)cc3)cc12] |
| 1348 | 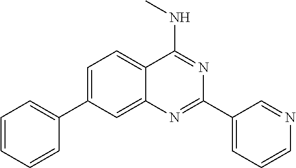 [SMILES: CNc1nc(-c2cccnc2)nc2cc(-c3ccccc3)ccc12] |
| 1349 | 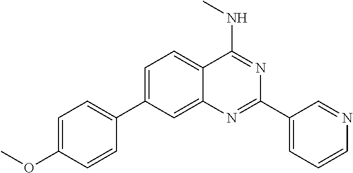 [SMILES: CNc1nc(-c2cccnc2)nc2cc(-c3ccc(OC)cc3)ccc12] |
| 1350 | 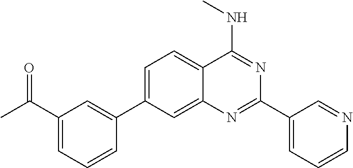 [SMILES: CNc1nc(-c2cccnc2)nc2cc(-c3cccc(C(C)=O)c3)ccc12] |
| 1351 | 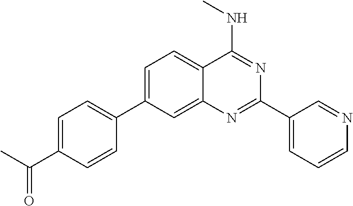 [SMILES: CNc1nc(-c2cccnc2)nc2cc(-c3ccc(C(C)=O)cc3)ccc12] |
| 1352 | 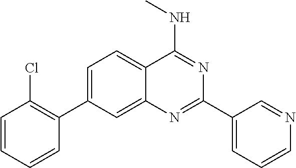 [SMILES: CNc1nc(-c2cccnc2)nc2cc(-c3ccccc3Cl)ccc12] |
| 1353 | 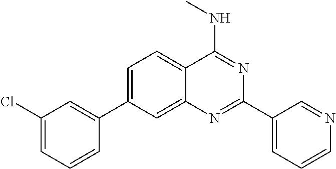 [SMILES: CNc1nc(-c2cccnc2)nc2cc(-c3cccc(Cl)c3)ccc12] |
| 1354 | 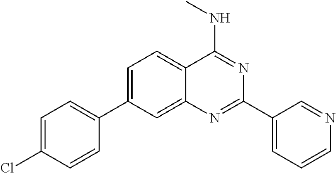 [SMILES: CNc1nc(-c2cccnc2)nc2cc(-c3ccc(Cl)cc3)ccc12] |
| 1355 | 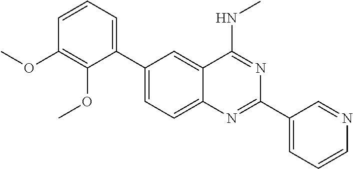 [SMILES: CNc1nc(-c2cccnc2)nc2ccc(-c3cccc(OC)c3OC)cc12] |
| 1356 | 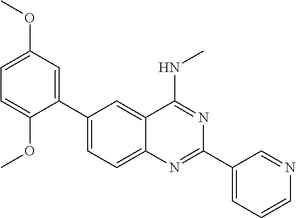 [SMILES: CNc1nc(-c2cccnc2)nc2ccc(-c3cc(OC)ccc3OC)cc12] |
| 1357 | 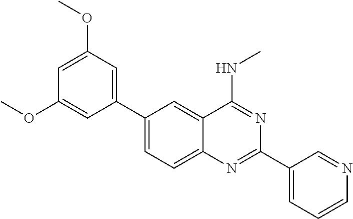 [SMILES: CNc1nc(-c2cccnc2)nc2ccc(-c3cc(OC)cc(OC)c3)cc12] |
| 1358 | 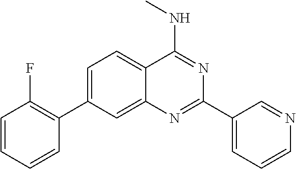 [SMILES: CNc1nc(-c2cccnc2)nc2cc(-c3ccccc3F)ccc12] |
| 1359 | 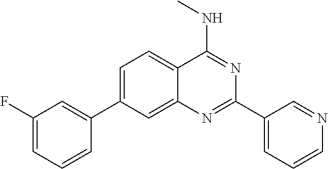 [SMILES: CNc1nc(-c2cccnc2)nc2cc(-c3cccc(F)c3)ccc12] |
| 1360 | 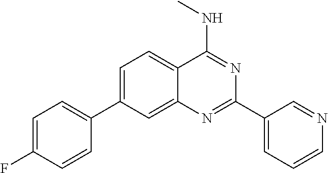 [SMILES: CNc1nc(-c2cccnc2)nc2cc(-c3ccc(F)cc3)ccc12] |
| 1361 | 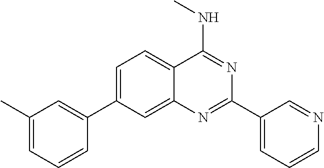 [SMILES: CNc1nc(-c2cccnc2)nc2cc(-c3cccc(C)c3)ccc12] |
| 1362 | 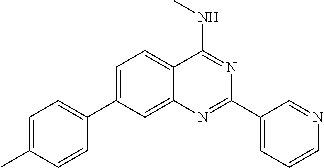 [SMILES: CNc1nc(-c2cccnc2)nc2cc(-c3ccc(C)cc3)ccc12] |
| 1363 | 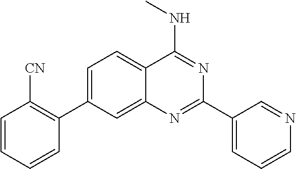 [SMILES: CNc1nc(-c2cccnc2)nc2cc(-c3ccccc3C#N)ccc12] |
| 1364 | 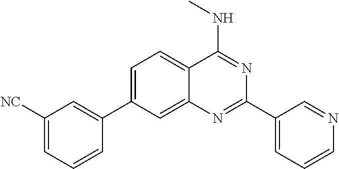 [SMILES: CNc1nc(-c2cccnc2)nc2cc(-c3cccc(C#N)c3)ccc12] |
| 1365 | 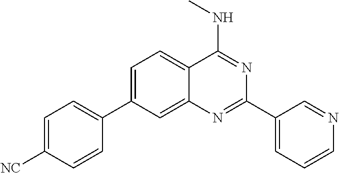 [SMILES: CNc1nc(-c2cccnc2)nc2cc(-c3ccc(C#N)cc3)ccc12] |
| 1366 | 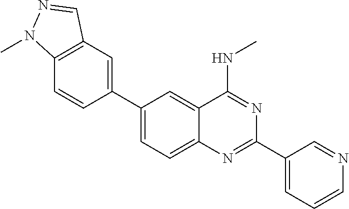 [SMILES: CNc1nc(-c2cccnc2)nc2ccc(-c3ccc4c(cnn4C)c3)cc12] |
| 1367 | 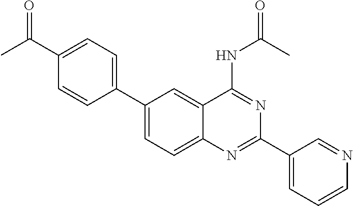 [SMILES: CC(=O)Nc1nc(-c2cccnc2)nc2ccc(-c3ccc(C(C)=O)cc3)cc12] |
| 1368 | 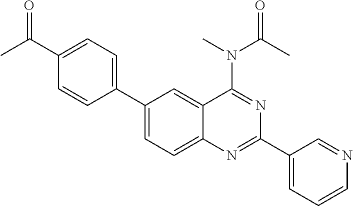 [SMILES: CC(=O)c1ccc(-c2ccc3nc(-c4cccnc4)nc(N(C)C(C)=O)c3c2)cc1] |
| 1369 | 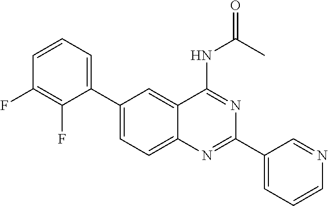 [SMILES: CC(=O)Nc1nc(-c2cccnc2)nc2ccc(-c3cccc(F)c3F)cc12] |
| 1370 | 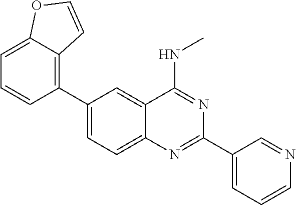 [SMILES: CNc1nc(-c2cccnc2)nc2ccc(-c3cccc4occc34)cc12] |
| 1371 | 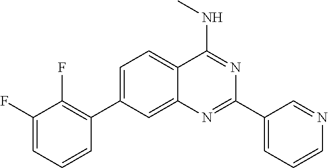 [SMILES: CNc1nc(-c2cccnc2)nc2cc(-c3cccc(F)c3F)ccc12] |
| 1372 | 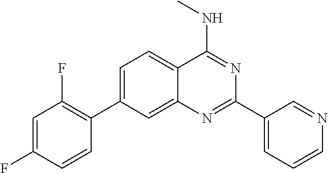 [SMILES: CNc1nc(-c2cccnc2)nc2cc(-c3ccc(F)cc3F)ccc12] |
| 1373 | 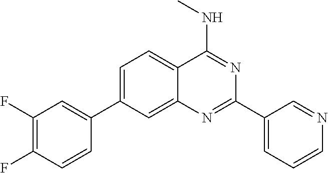 [SMILES: CNc1nc(-c2cccnc2)nc2cc(-c3ccc(F)c(F)c3)ccc12] |
| 1374 | 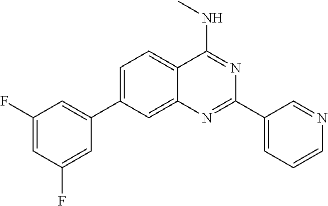 [SMILES: CNc1nc(-c2cccnc2)nc2cc(-c3cc(F)cc(F)c3)ccc12] |
| 1375 | 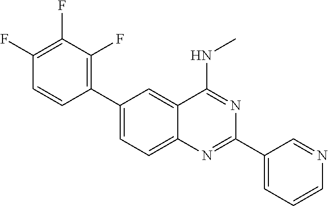 [SMILES: CNc1nc(-c2cccnc2)nc2ccc(-c3ccc(F)c(F)c3F)cc12] |
| 1376 | 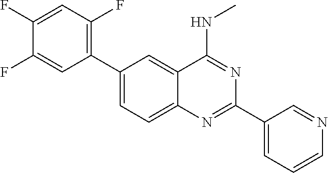 [SMILES: CNc1nc(-c2cccnc2)nc2ccc(-c3cc(F)c(F)cc3F)cc12] |
| 1377 | 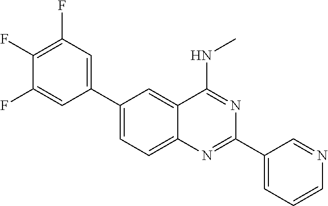 [SMILES: CNc1nc(-c2cccnc2)nc2ccc(-c3cc(F)c(F)c(F)c3)cc12] |
| 1378 | 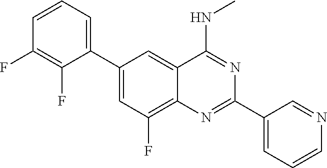 [SMILES: CNc1nc(-c2cccnc2)nc2c(F)cc(-c3cccc(F)c3F)cc12] |
| 1379 | 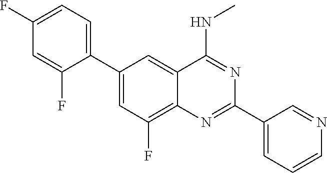 [SMILES: CNc1nc(-c2cccnc2)nc2c(F)cc(-c3ccc(F)cc3F)cc12] |
| 1380 | 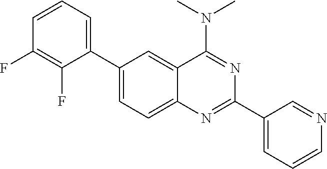 [SMILES: CN(C)c1nc(-c2cccnc2)nc2ccc(-c3cccc(F)c3F)cc12] |
| 1381 | 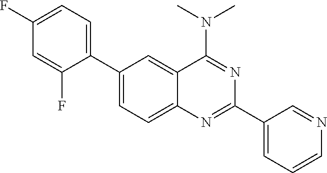 [SMILES: CN(C)c1nc(-c2cccnc2)nc2ccc(-c3ccc(F)cc3F)cc12] |
| 1382 | 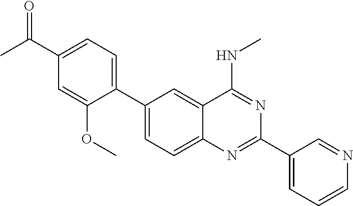 [SMILES: CNc1nc(-c2cccnc2)nc2ccc(-c3ccc(C(C)=O)cc3OC)cc12] |
| 1383 | 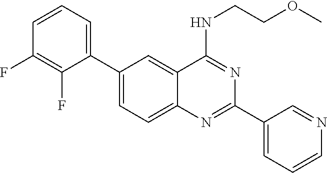 [SMILES: COCCNc1nc(-c2cccnc2)nc2ccc(-c3cccc(F)c3F)cc12] |
| 1384 | 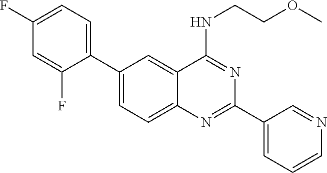 [SMILES: COCCNc1nc(-c2cccnc2)nc2ccc(-c3ccc(F)cc3F)cc12] |
| 1385 | 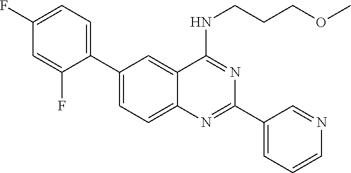 [SMILES: COCCCNc1nc(-c2cccnc2)nc2ccc(-c3ccc(F)cc3F)cc12] |
| 1386 | 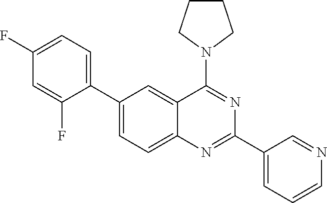 [SMILES: Fc1ccc(-c2ccc3nc(-c4cccnc4)nc(N4CCCC4)c3c2)c(F)c1] |
| 1387 | 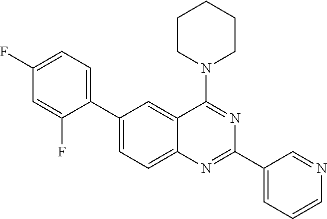 [SMILES: Fc1ccc(-c2ccc3nc(-c4cccnc4)nc(N4CCCCC4)c3c2)c(F)c1] |
| 1388 | 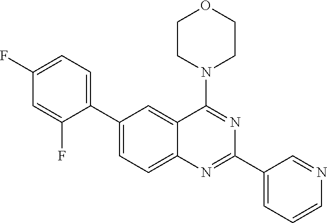 [SMILES: Fc1ccc(-c2ccc3nc(-c4cccnc4)nc(N4CCOCC4)c3c2)c(F)c1] |
| 1389 | 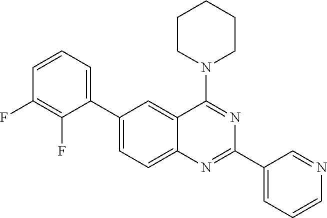 [SMILES: Fc1cccc(-c2ccc3nc(-c4cccnc4)nc(N4CCCCC4)c3c2)c1F] |
| 1390 | 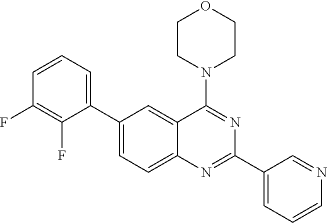 [SMILES: Fc1cccc(-c2ccc3nc(-c4cccnc4)nc(N4CCOCC4)c3c2)c1F] |
| 1391 | 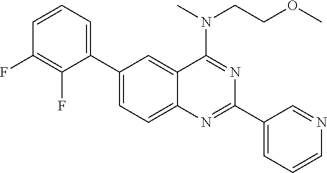 [SMILES: COCCN(C)c1nc(-c2cccnc2)nc2ccc(-c3cccc(F)c3F)cc12] |
| 1392 | 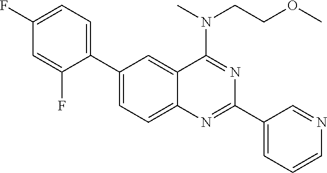 [SMILES: COCCN(C)c1nc(-c2cccnc2)nc2ccc(-c3ccc(F)cc3F)cc12] |
| 1393 | 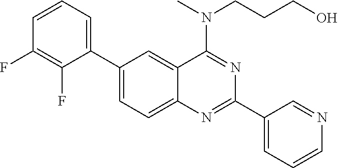 [SMILES: CN(CCCO)c1nc(-c2cccnc2)nc2ccc(-c3cccc(F)c3F)cc12] |
| 1394 | 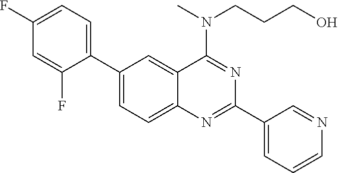 [SMILES: CN(CCCO)c1nc(-c2cccnc2)nc2ccc(-c3ccc(F)cc3F)cc12] |
| 1395 | 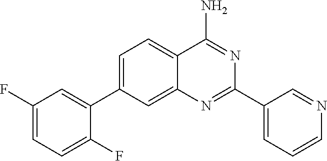 [SMILES: Nc1nc(-c2cccnc2)nc2cc(-c3cc(F)ccc3F)ccc12] |
| 1396 | 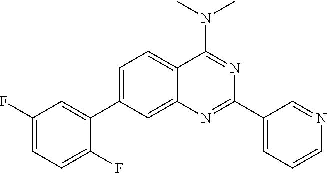 [SMILES: CN(C)c1nc(-c2cccnc2)nc2cc(-c3cc(F)ccc3F)ccc12] |
| 1397 | 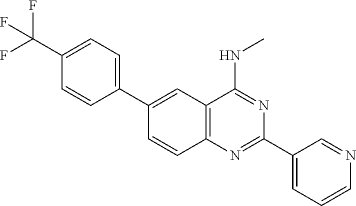 [SMILES: CNc1nc(-c2cccnc2)nc2ccc(-c3ccc(C(F)(F)F)cc3)cc12] |
| 1398 | 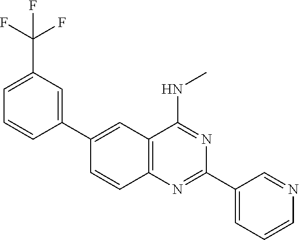 [SMILES: CNc1nc(-c2cccnc2)nc2ccc(-c3cccc(C(F)(F)F)c3)cc12] |
| 1399 | 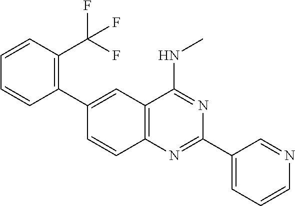 [SMILES: CNc1nc(-c2cccnc2)nc2ccc(-c3ccccc3C(F)(F)F)cc12] |
| 1400 | 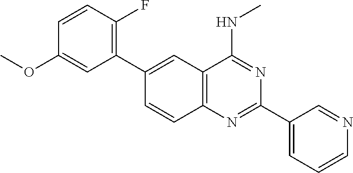 [SMILES: CNc1nc(-c2cccnc2)nc2ccc(-c3cc(OC)ccc3F)cc12] |
| 1401 | 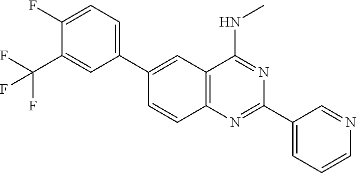 [SMILES: CNc1nc(-c2cccnc2)nc2ccc(-c3ccc(F)c(C(F)(F)F)c3)cc12] |
| 1402 | 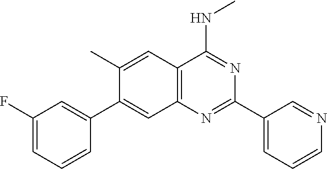 [SMILES: CNc1nc(-c2cccnc2)nc2cc(-c3cccc(F)c3)c(C)cc12] |
| 1403 | 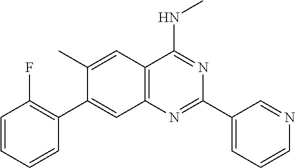 [SMILES: CNc1nc(-c2cccnc2)nc2cc(-c3ccccc3F)c(C)cc12] |
| 1404 | 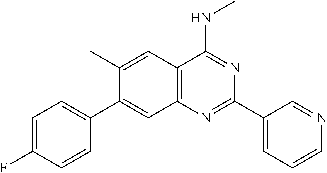 [SMILES: CNc1nc(-c2cccnc2)nc2cc(-c3ccc(F)cc3)c(C)cc12] |
| 1405 | 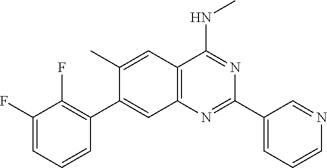 [SMILES: CNc1nc(-c2cccnc2)nc2cc(-c3cccc(F)c3F)c(C)cc12] |
| 1406 | 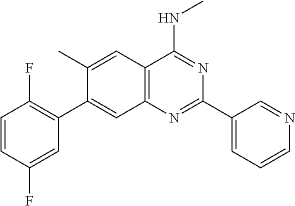 [SMILES: CNc1nc(-c2cccnc2)nc2cc(-c3cc(F)ccc3F)c(C)cc12] |
| 1407 | 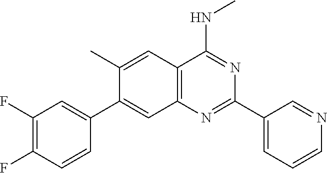 [SMILES: CNc1nc(-c2cccnc2)nc2cc(-c3ccc(F)c(F)c3)c(C)cc12] |
| 1408 | 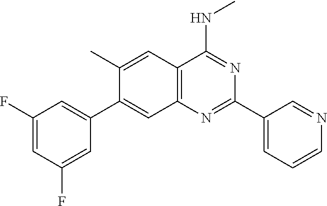 [SMILES: CNc1nc(-c2cccnc2)nc2cc(-c3cc(F)cc(F)c3)c(C)cc12] |
| 1409 | 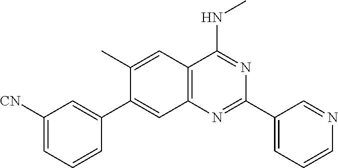 |
| 1410 | 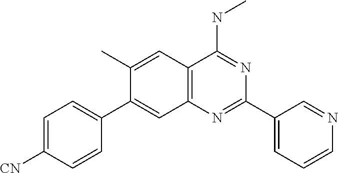 |
| 1411 | 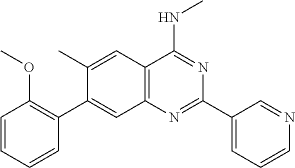 [SMILES: CNc1nc(-c2cccnc2)nc2cc(-c3ccccc3OC)c(C)cc12] |
| 1412 | 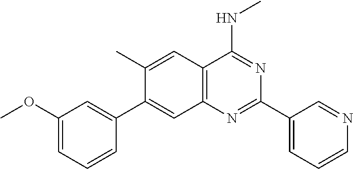 [SMILES: CNc1nc(-c2cccnc2)nc2cc(-c3cccc(OC)c3)c(C)cc12] |
| 1413 | 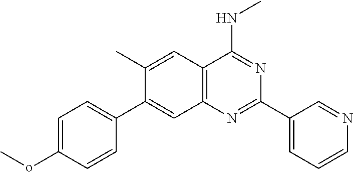 |
| 1414 | 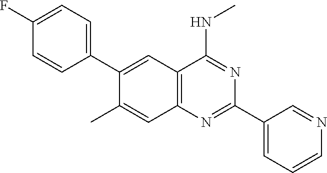 [SMILES: CNc1nc(-c2cccnc2)nc2cc(C)c(-c3ccc(F)cc3)cc12] |
| 1415 | 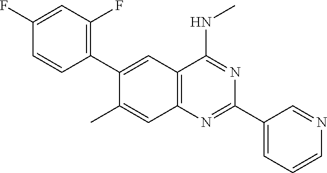 [SMILES: CNc1nc(-c2cccnc2)nc2cc(C)c(-c3ccc(F)cc3F)cc12] |
| 1416 | 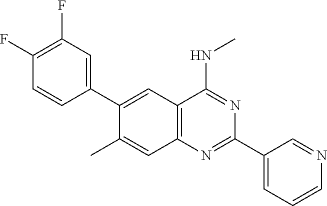 [SMILES: CNc1nc(-c2cccnc2)nc2cc(C)c(-c3ccc(F)c(F)c3)cc12] |
| 1417 | 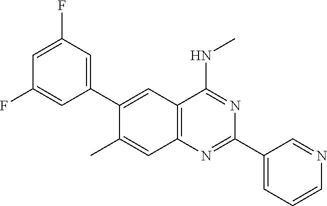 [SMILES: CNc1nc(-c2cccnc2)nc2cc(C)c(-c3cc(F)cc(F)c3)cc12] |
| 1418 | 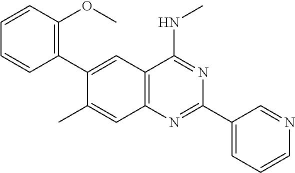 [SMILES: CNc1nc(-c2cccnc2)nc2cc(C)c(-c3ccccc3OC)cc12] |
| 1419 | 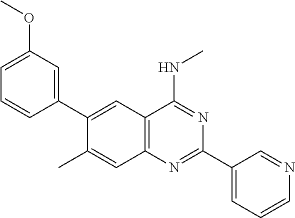 [SMILES: CNc1nc(-c2cccnc2)nc2cc(C)c(-c3cccc(OC)c3)cc12] |
| 1420 | 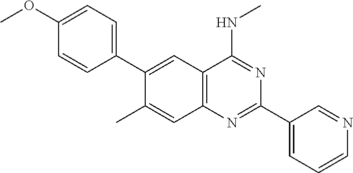 [SMILES: CNc1nc(-c2cccnc2)nc2cc(C)c(-c3ccc(OC)cc3)cc12] |
| 1421 | 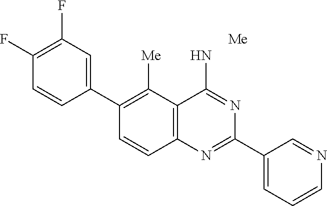 [SMILES: Cc1c(-c2ccc(F)c(F)c2)ccc2nc(-c3cccnc3)nc(N)c12 ; "Me"] |
| 1422 | 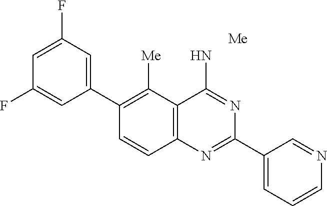 [SMILES: Cc1c(-c2cc(F)cc(F)c2)ccc2nc(-c3cccnc3)nc(N)c12 ; "Me"] |
| 1423 | 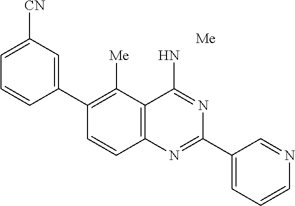 [SMILES: Cc1c(-c2cccc(C#N)c2)ccc2nc(-c3cccnc3)nc(N)c12 ; "Me"] |
| 1424 | 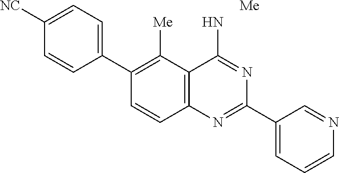 [SMILES: Cc1c(-c2ccc(C#N)cc2)ccc2nc(-c3cccnc3)nc(N)c12 ; "Me"] |
| 1425 | 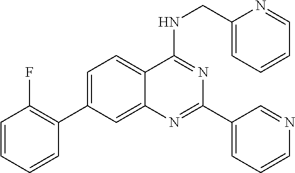 [SMILES: Fc1ccccc1-c1ccc2c(NCc3ccccn3)nc(-c3cccnc3)nc2c1] |
| 1426 | 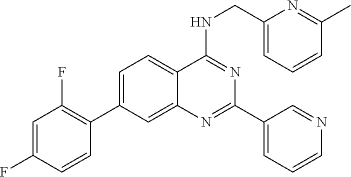 [SMILES: Cc1cccc(CNc2nc(-c3cccnc3)nc3cc(-c4ccc(F)cc4F)ccc23)n1] |
| 1427 | 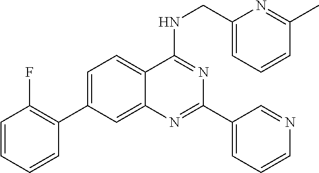 [SMILES: Cc1cccc(CNc2nc(-c3cccnc3)nc3cc(-c4ccccc4F)ccc23)n1] |
| 1428 | 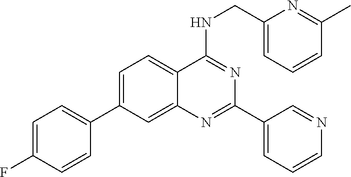 [SMILES: Cc1cccc(CNc2nc(-c3cccnc3)nc3cc(-c4ccc(F)cc4)ccc23)n1] |
| 1429 | 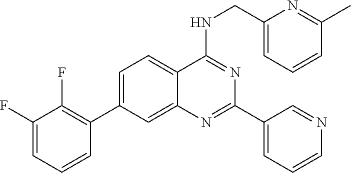 [SMILES: Cc1cccc(CNc2nc(-c3cccnc3)nc3cc(-c4cccc(F)c4F)ccc23)n1] |
| 1430 | 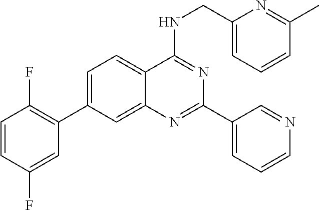 [SMILES: Cc1cccc(CNc2nc(-c3cccnc3)nc3cc(-c4cc(F)ccc4F)ccc23)n1] |
| 1431 | 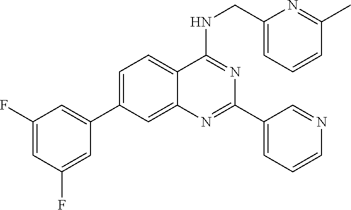 [SMILES: Cc1cccc(CNc2nc(-c3cccnc3)nc3cc(-c4cc(F)cc(F)c4)ccc23)n1] |
| 1432 | 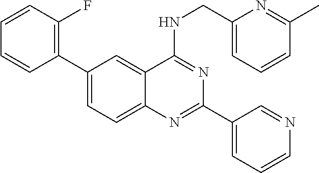 [SMILES: Cc1cccc(CNc2nc(-c3cccnc3)nc3ccc(-c4ccccc4F)cc23)n1] |
| 1433 | 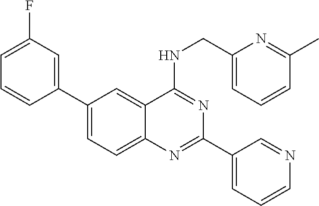 [SMILES: Cc1cccc(CNc2nc(-c3cccnc3)nc3ccc(-c4cccc(F)c4)cc23)n1] |
| 1434 | 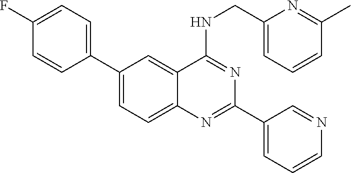 [SMILES: Cc1cccc(CNc2nc(-c3cccnc3)nc3ccc(-c4ccc(F)cc4)cc23)n1] |
| 1435 | 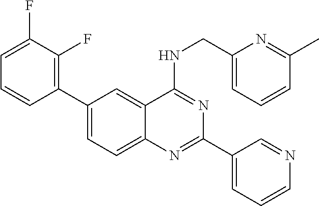 [SMILES: Cc1cccc(CNc2nc(-c3cccnc3)nc3ccc(-c4cccc(F)c4F)cc23)n1] |
| 1436 | 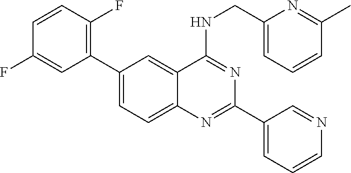 [SMILES: Cc1cccc(CNc2nc(-c3cccnc3)nc3ccc(-c4cc(F)ccc4F)cc23)n1] |
| 1437 | 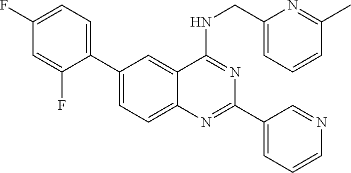 [SMILES: Cc1cccc(CNc2nc(-c3cccnc3)nc3ccc(-c4ccc(F)cc4F)cc23)n1] |
| 1438 | 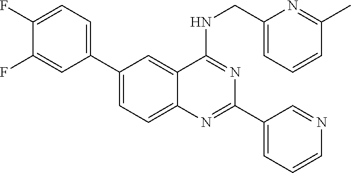 [SMILES: Cc1cccc(CNc2nc(-c3cccnc3)nc3ccc(-c4ccc(F)c(F)c4)cc23)n1] |
| 1439 | 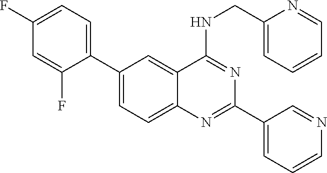 [SMILES: Fc1ccc(-c2ccc3nc(-c4cccnc4)nc(NCc4ccccn4)c3c2)c(F)c1] |
| 1440 | 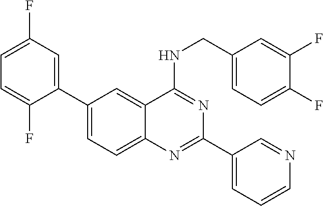 [SMILES: Fc1ccc(F)c(-c2ccc3nc(-c4cccnc4)nc(NCc4ccc(F)c(F)c4)c3c2)c1] |
| 1441 | 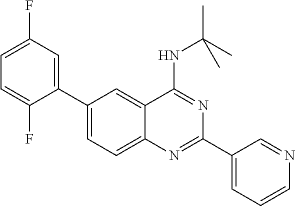 [SMILES: CC(C)(C)Nc1nc(-c2cccnc2)nc2ccc(-c3cc(F)ccc3F)cc12] |
| 1442 | 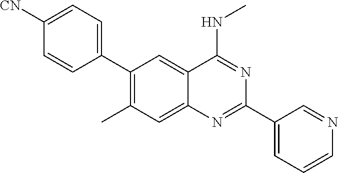 |
| 1443 | 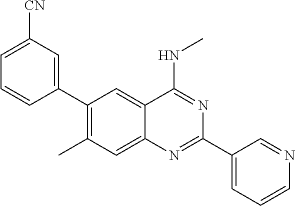 [SMILES: CNc1nc(-c2cccnc2)nc2cc(C)c(-c3cccc(C#N)c3)cc12] |
| 1444 | 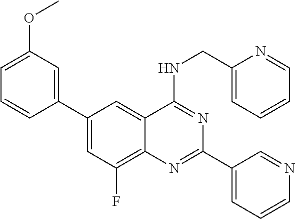 [SMILES: COc1cccc(-c2cc(F)c3nc(-c4cccnc4)nc(NCc4ccccn4)c3c2)c1] |
| 1445 | 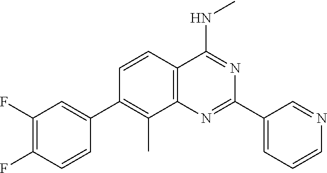 [SMILES: CNc1nc(-c2cccnc2)nc2c(C)c(-c3ccc(F)c(F)c3)ccc12] |
| 1446 | 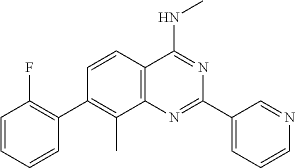 [SMILES: CNc1nc(-c2cccnc2)nc2c(C)c(-c3ccccc3F)ccc12] |
| 1447 | 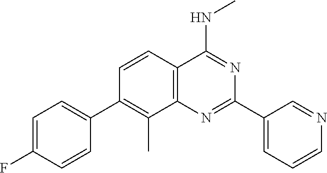 [SMILES: CNc1nc(-c2cccnc2)nc2c(C)c(-c3ccc(F)cc3)ccc12] |
| 1448 | 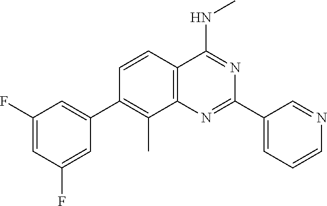 [SMILES: CNc1nc(-c2cccnc2)nc2c(C)c(-c3cc(F)cc(F)c3)ccc12] |
| 1449 | 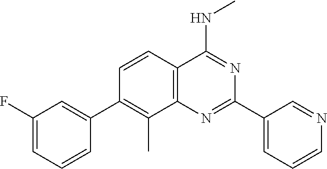 [SMILES: CNc1nc(-c2cccnc2)nc2c(C)c(-c3cccc(F)c3)ccc12] |
| 1450 | 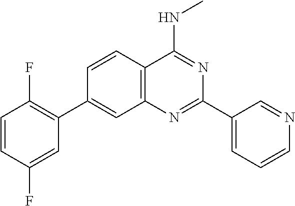 [SMILES: CNc1nc(-c2cccnc2)nc2cc(-c3cc(F)ccc3F)ccc12] |
| 1451 | 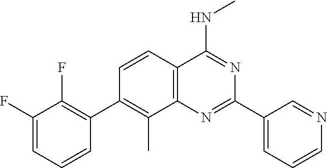 [SMILES: CNc1nc(-c2cccnc2)nc2c(C)c(-c3cccc(F)c3F)ccc12] |
| 1452 | 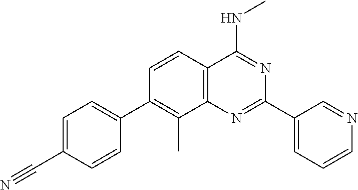 [SMILES: CNc1nc(-c2cccnc2)nc2c(C)c(-c3ccc(C#N)cc3)ccc12] |
| 1453 | 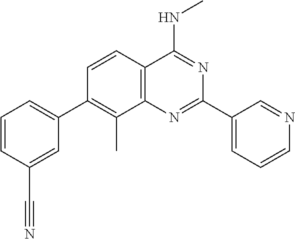 [SMILES: CNc1nc(-c2cccnc2)nc2c(C)c(-c3cccc(C#N)c3)ccc12] |
| 1454 | 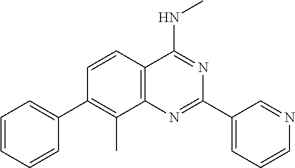 [SMILES: CNc1nc(-c2cccnc2)nc2c(C)c(-c3ccccc3)ccc12] |
| 1455 | 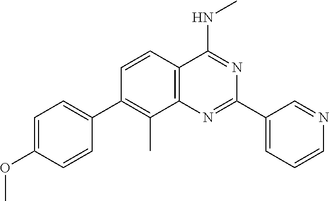 [SMILES: CNc1nc(-c2cccnc2)nc2c(C)c(-c3ccc(OC)cc3)ccc12] |
| 1456 | 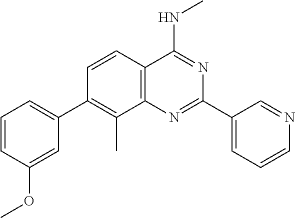 [SMILES: CNc1nc(-c2cccnc2)nc2c(C)c(-c3cccc(OC)c3)ccc12] |
| 1457 | 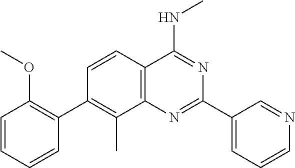 [SMILES: CNc1nc(-c2cccnc2)nc2c(C)c(-c3ccccc3OC)ccc12] |
| 1458 | 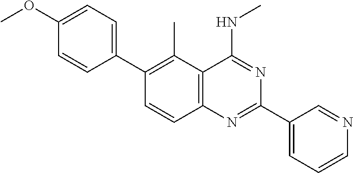 [SMILES: CNc1nc(-c2cccnc2)nc2ccc(-c3ccc(OC)cc3)c(C)c12] |
| 1459 | 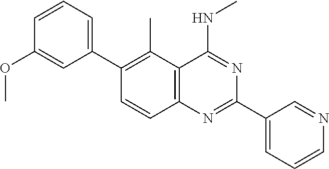 [SMILES: CNc1nc(-c2cccnc2)nc2ccc(-c3cccc(OC)c3)c(C)c12] |
| 1460 | 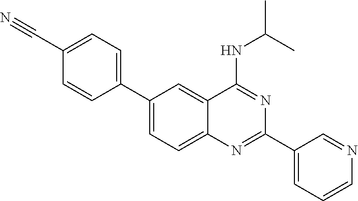 [SMILES: CC(C)Nc1nc(-c2cccnc2)nc2ccc(-c3ccc(C#N)cc3)cc12] |
| 1461 | 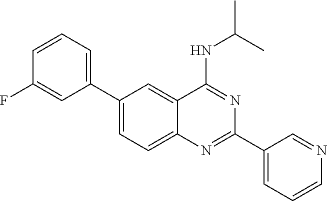 [SMILES: CC(C)Nc1nc(-c2cccnc2)nc2ccc(-c3cccc(F)c3)cc12] |
| 1462 | 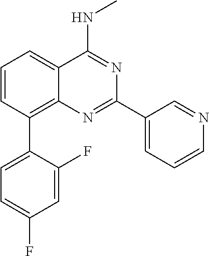 [SMILES: CNc1nc(-c2cccnc2)nc2c(-c3ccc(F)cc3F)cccc12] |
| 1463 | 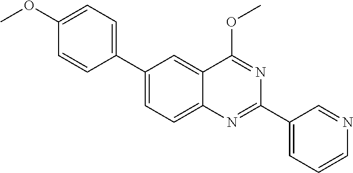 [SMILES: COc1ccc(-c2ccc3nc(-c4cccnc4)nc(OC)c3c2)cc1] |
| 1464 | 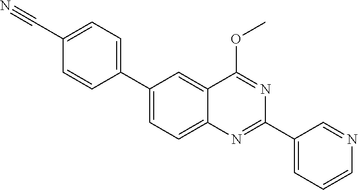 [SMILES: COc1nc(-c2cccnc2)nc2ccc(-c3ccc(C#N)cc3)cc12] |
| 1465 | 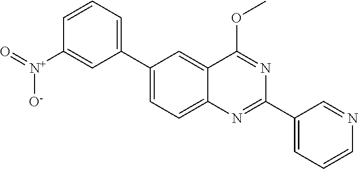 [SMILES: COc1nc(-c2cccnc2)nc2ccc(-c3cccc([N+](=O)[O-])c3)cc12] |
| 1466 | 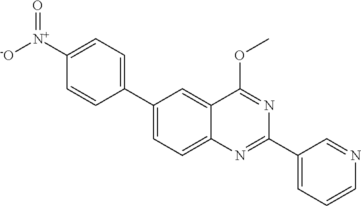 |
| 1467 | 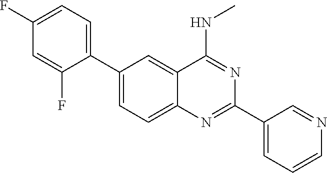 [SMILES: CNc1nc(-c2cccnc2)nc2ccc(-c3ccc(F)cc3F)cc12] |
| 1468 | 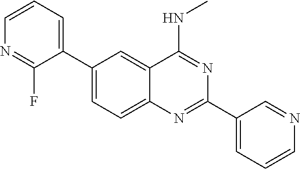 [SMILES: CNc1nc(-c2cccnc2)nc2ccc(-c3cccnc3F)cc12] |
| 1469 | 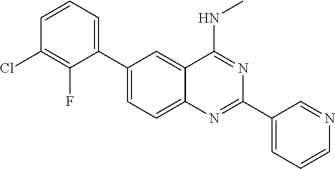 [SMILES: CNc1nc(-c2cccnc2)nc2ccc(-c3cccc(Cl)c3F)cc12] |
| 1470 | 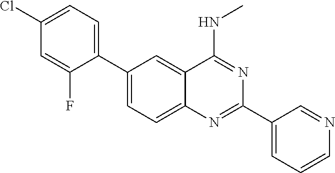 [SMILES: CNc1nc(-c2cccnc2)nc2ccc(-c3ccc(Cl)cc3F)cc12] |
| 1471 | 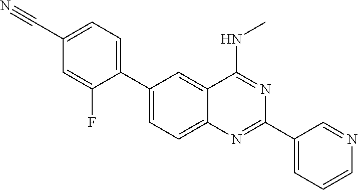 [SMILES: CNc1nc(-c2cccnc2)nc2ccc(-c3ccc(C#N)cc3F)cc12] |
| 1472 | 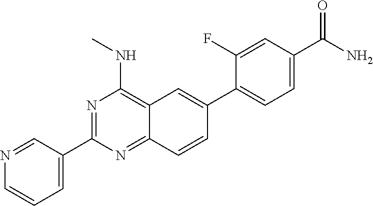 [SMILES: CNc1nc(-c2cccnc2)nc2ccc(-c3ccc(C(N)=O)cc3F)cc12] |
| 1473 | 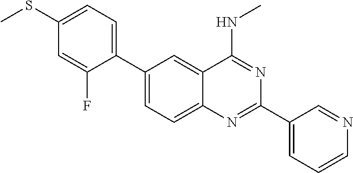 [SMILES: CNc1nc(-c2cccnc2)nc2ccc(-c3ccc(SC)cc3F)cc12] |
| 1474 | 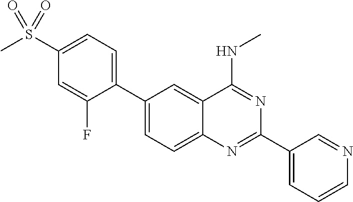 [SMILES: CNc1nc(-c2cccnc2)nc2ccc(-c3ccc(S(C)(=O)=O)cc3F)cc12] |
| 1475 | 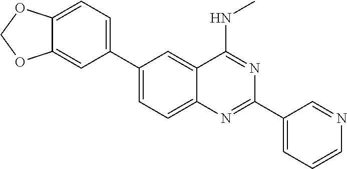 [SMILES: CNc1nc(-c2cccnc2)nc2ccc(-c3ccc4c(c3)OCO4)cc12] |
| 1476 | 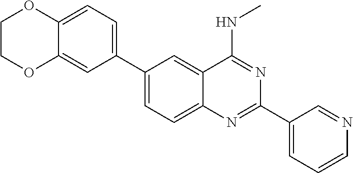 [SMILES: CNc1nc(-c2cccnc2)nc2ccc(-c3ccc4c(c3)OCCO4)cc12] |
| 1477 | 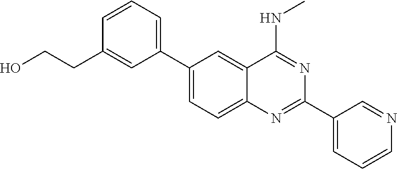 [SMILES: CNc1nc(-c2cccnc2)nc2ccc(-c3cccc(CCO)c3)cc12] |
| 1478 | 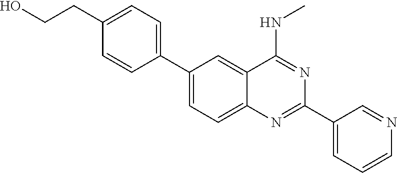 [SMILES: CNc1nc(-c2cccnc2)nc2ccc(-c3ccc(CCO)cc3)cc12] |
| 1479 | 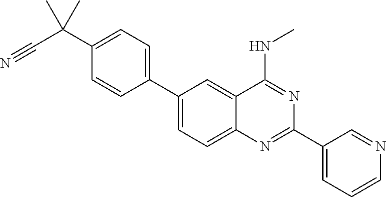 [SMILES: CNc1nc(-c2cccnc2)nc2ccc(-c3ccc(C(C)(C)C#N)cc3)cc12] |
| 1480 | 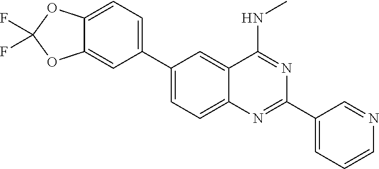 [SMILES: CNc1nc(-c2cccnc2)nc2ccc(-c3ccc4c(c3)OC(F)(F)O4)cc12] |
| 1481 | 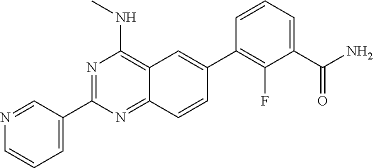 [SMILES: CNc1nc(-c2cccnc2)nc2ccc(-c3cccc(C(N)=O)c3F)cc12] |
| 1482 | 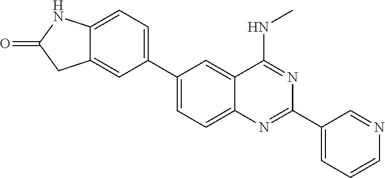 [SMILES: CNc1nc(-c2cccnc2)nc2ccc(-c3ccc4c(c3)CC(=O)N4)cc12] |
| 1483 | 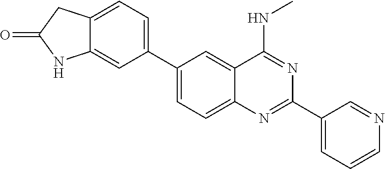 [SMILES: CNc1nc(-c2cccnc2)nc2ccc(-c3ccc4c(c3)NC(=O)C4)cc12] |
| 1484 | 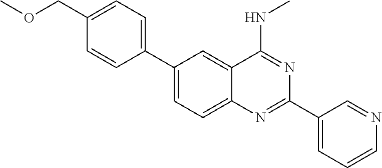 [SMILES: CNc1nc(-c2cccnc2)nc2ccc(-c3ccc(COC)cc3)cc12] |
| 1485 | 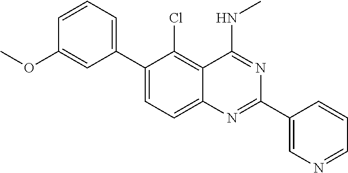 [SMILES: CNc1nc(-c2cccnc2)nc2ccc(-c3cccc(OC)c3)c(Cl)c12] |
| 1486 | 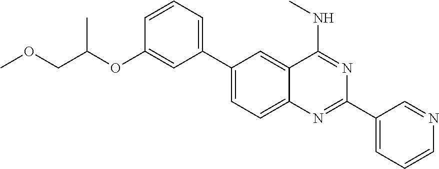 [SMILES: CNc1nc(-c2cccnc2)nc2ccc(-c3cccc(OC(C)COC)c3)cc12] |
| 1487 | 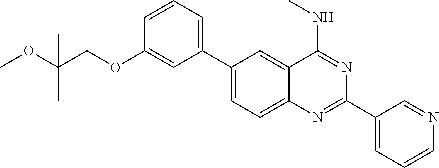 [SMILES: CNc1nc(-c2cccnc2)nc2ccc(-c3cccc(OCC(C)(C)OC)c3)cc12] |
| 1488 | 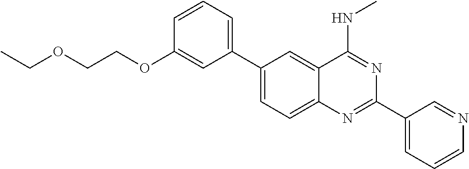 [SMILES: CCOCCOc1cccc(-c2ccc3nc(-c4cccnc4)nc(NC)c3c2)c1] |
| 1489 | 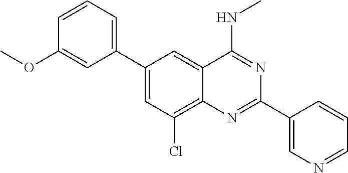 [SMILES: CNc1nc(-c2cccnc2)nc2c(Cl)cc(-c3cccc(OC)c3)cc12] |
| 1490 | 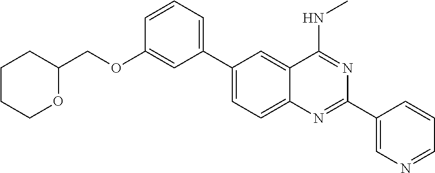 [SMILES: CNc1nc(-c2cccnc2)nc2ccc(-c3cccc(OCC4CCCCO4)c3)cc12] |
| 1492 | 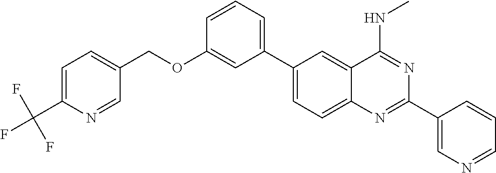 [SMILES: CNc1nc(-c2cccnc2)nc2ccc(-c3cccc(OCc4ccc(C(F)(F)F)nc4)c3)cc12] |
| 1493 | 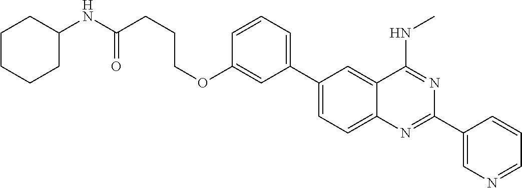 [SMILES: CNc1nc(-c2cccnc2)nc2ccc(-c3cccc(OCCCC(=O)NC4CCCCC4)c3)cc12] |
| 1494 | 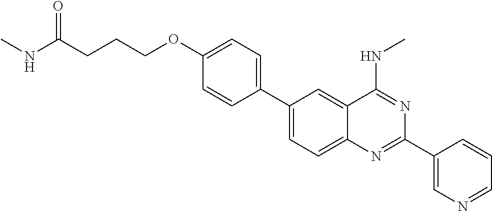 [SMILES: CNC(=O)CCCOc1ccc(-c2ccc3nc(-c4cccnc4)nc(NC)c3c2)cc1] |
| 1495 | 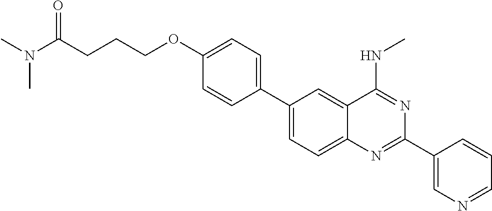 [SMILES: CNc1nc(-c2cccnc2)nc2ccc(-c3ccc(OCCCC(=O)N(C)C)cc3)cc12] |
| 1496 | 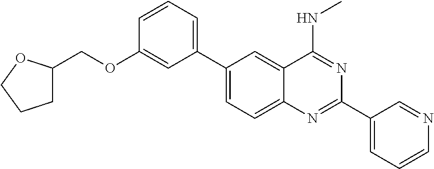 [SMILES: CNc1nc(-c2cccnc2)nc2ccc(-c3cccc(OCC4CCCO4)c3)cc12] |
| 1497 | 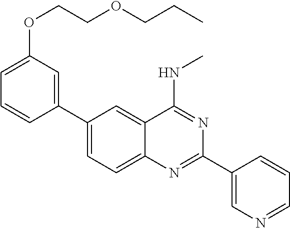 [SMILES: CCCOCCOc1cccc(-c2ccc3nc(-c4cccnc4)nc(NC)c3c2)c1] |
| 1498 | 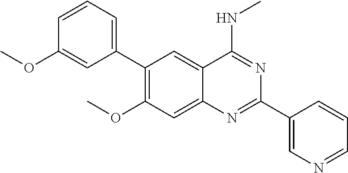 [SMILES: CNc1nc(-c2cccnc2)nc2cc(OC)c(-c3cccc(OC)c3)cc12] |
| 1499 | 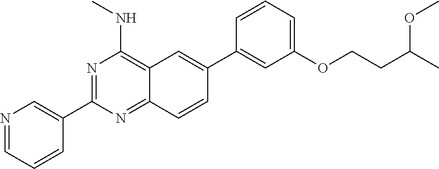 [SMILES: CNc1nc(-c2cccnc2)nc2ccc(-c3cccc(OCCC(C)OC)c3)cc12] |
| 1500 | 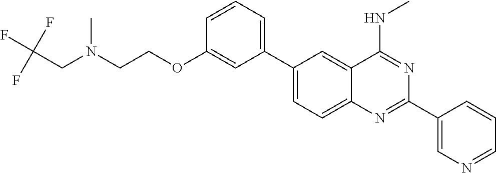 [SMILES: CNc1nc(-c2cccnc2)nc2ccc(-c3cccc(OCCN(C)CC(F)(F)F)c3)cc12] |
| 1501 | 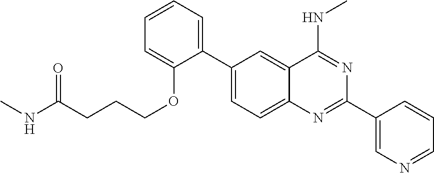 [SMILES: CNC(=O)CCCOc1ccccc1-c1ccc2nc(-c3cccnc3)nc(NC)c2c1] |
| 1502 | 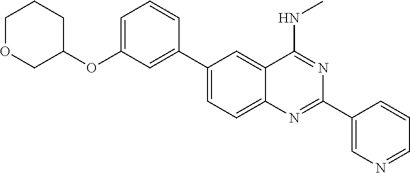 [SMILES: CNc1nc(-c2cccnc2)nc2ccc(-c3cccc(OC4CCCOC4)c3)cc12] |
| 1503 | 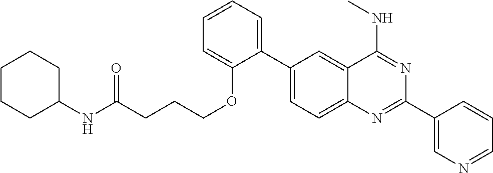 [SMILES: CNc1nc(-c2cccnc2)nc2ccc(-c3ccccc3OCCCC(=O)NC3CCCCC3)cc12] |
| 1504 | 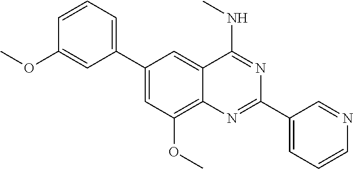 [SMILES: CNc1nc(-c2cccnc2)nc2c(OC)cc(-c3cccc(OC)c3)cc12] |
| 1505 | 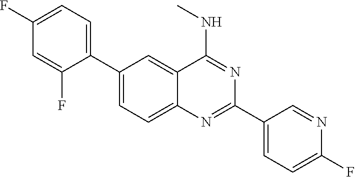 [SMILES: CNc1nc(-c2ccc(F)nc2)nc2ccc(-c3ccc(F)cc3F)cc12] |
| 1506 | 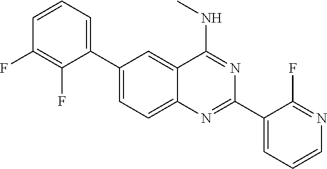 [SMILES: CNc1nc(-c2cccnc2F)nc2ccc(-c3cccc(F)c3F)cc12] |
| 1507 | 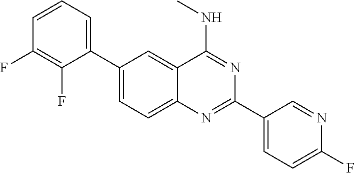 [SMILES: CNc1nc(-c2ccc(F)nc2)nc2ccc(-c3cccc(F)c3F)cc12] |
| 1508 | 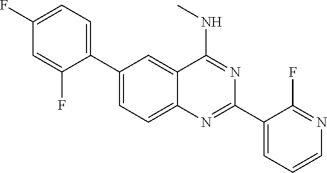 [SMILES: CNc1nc(-c2cccnc2F)nc2ccc(-c3ccc(F)cc3F)cc12] |
| 1509 | 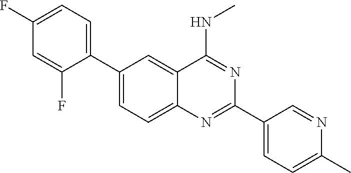 [SMILES: CNc1nc(-c2ccc(C)nc2)nc2ccc(-c3ccc(F)cc3F)cc12] |
| 1510 | 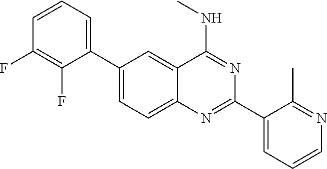 [SMILES: CNc1nc(-c2cccnc2C)nc2ccc(-c3cccc(F)c3F)cc12] |
| 1511 | 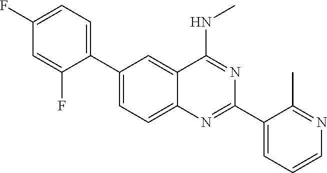 [SMILES: CNc1nc(-c2cccnc2C)nc2ccc(-c3ccc(F)cc3F)cc12] |
| 1512 | 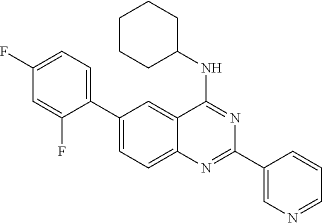 [SMILES: Fc1ccc(-c2ccc3nc(-c4cccnc4)nc(NC4CCCCC4)c3c2)c(F)c1] |
| 1513 | 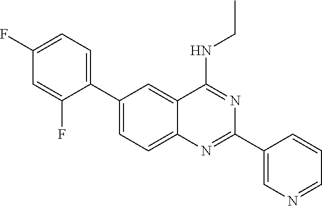 [SMILES: CCNc1nc(-c2cccnc2)nc2ccc(-c3ccc(F)cc3F)cc12] |
| 1514 | 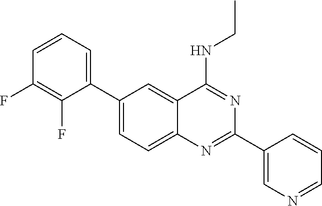 [SMILES: CCNc1nc(-c2cccnc2)nc2ccc(-c3cccc(F)c3F)cc12] |
| 1515 | 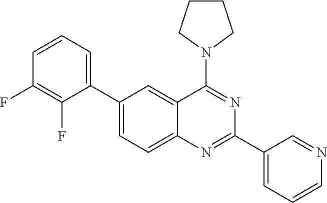 [SMILES: Fc1cccc(-c2ccc3nc(-c4cccnc4)nc(N4CCCC4)c3c2)c1F] |
| 1516 | 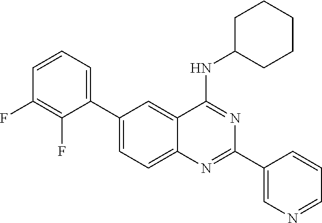 [SMILES: Fc1cccc(-c2ccc3nc(-c4cccnc4)nc(NC4CCCCC4)c3c2)c1F] |
| 1517 | 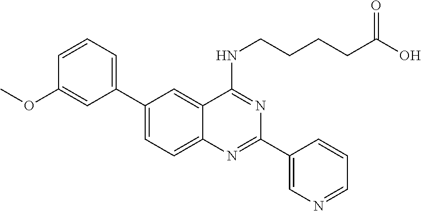 [SMILES: COc1cccc(-c2ccc3nc(-c4cccnc4)nc(NCCCCC(=O)O)c3c2)c1] |
| 1518 | 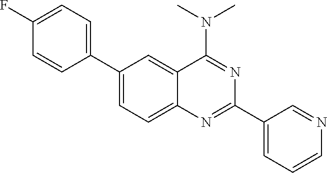 [SMILES: CN(C)c1nc(-c2cccnc2)nc2ccc(-c3ccc(F)cc3)cc12] |
| 1519 | 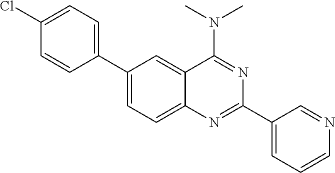 [SMILES: CN(C)c1nc(-c2cccnc2)nc2ccc(-c3ccc(Cl)cc3)cc12] |
| 1520 | 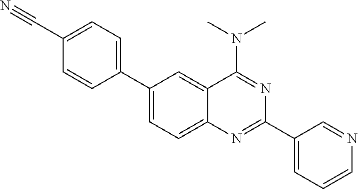 [SMILES: CN(C)c1nc(-c2cccnc2)nc2ccc(-c3ccc(C#N)cc3)cc12] |
| 1521 | 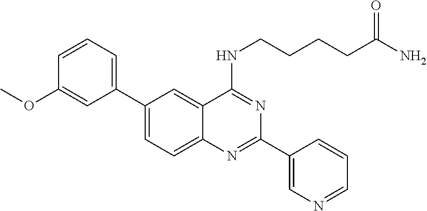 [SMILES: COc1cccc(-c2ccc3nc(-c4cccnc4)nc(NCCCCC(N)=O)c3c2)c1] |
| 1522 | 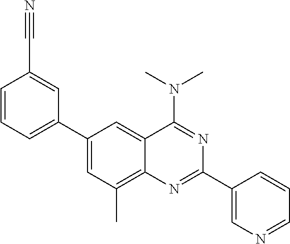 [SMILES: Cc1cc(-c2cccc(C#N)c2)cc2c(N(C)C)nc(-c3cccnc3)nc12] |
| 1523 | 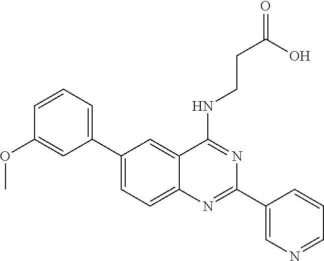 [SMILES: COc1cccc(-c2ccc3nc(-c4cccnc4)nc(NCCC(=O)O)c3c2)c1] |
| 1524 | 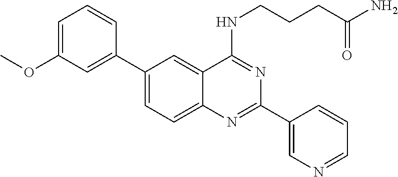 [SMILES: COc1cccc(-c2ccc3nc(-c4cccnc4)nc(NCCCC(N)=O)c3c2)c1] |
| 1525 | 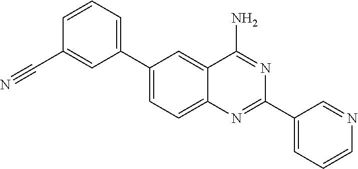 [SMILES: N#Cc1cccc(-c2ccc3nc(-c4cccnc4)nc(N)c3c2)c1] |
| 1526 | 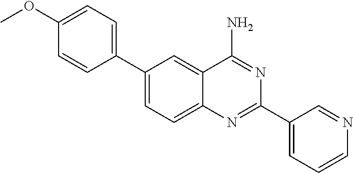 [SMILES: COc1ccc(-c2ccc3nc(-c4cccnc4)nc(N)c3c2)cc1] |
| 1527 | 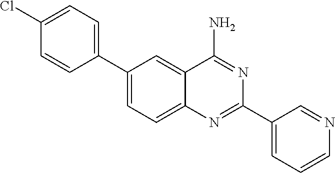 [SMILES: Nc1nc(-c2cccnc2)nc2ccc(-c3ccc(Cl)cc3)cc12] |
| 1528 | 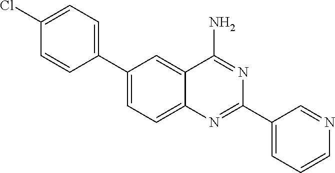 [SMILES: Nc1nc(-c2cccnc2)nc2ccc(-c3ccc(Cl)cc3)cc12] |
| 1529 | 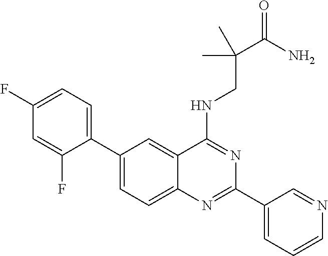 [SMILES: CC(C)(CNc1nc(-c2cccnc2)nc2ccc(-c3ccc(F)cc3F)cc12)C(N)=O] |
| 1530 | 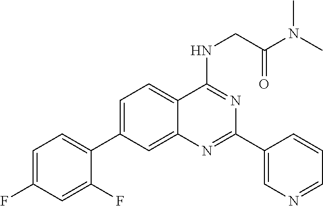 [SMILES: CN(C)C(=O)CNc1nc(-c2cccnc2)nc2cc(-c3ccc(F)cc3F)ccc12] |
| 1531 | 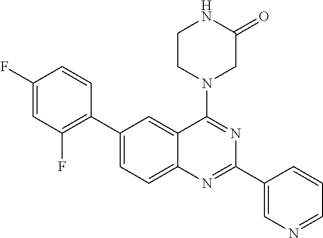 [SMILES: O=C1CN(c2nc(-c3cccnc3)nc3ccc(-c4ccc(F)cc4F)cc23)CCN1] |
| 1532 | 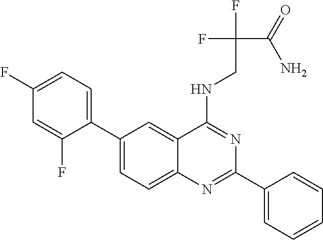 [SMILES: NC(=O)C(F)(F)CNc1nc(-c2ccccc2)nc2ccc(-c3ccc(F)cc3F)cc12] |
| 1533 | 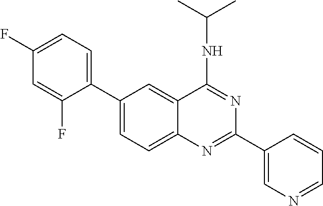 [SMILES: CC(C)Nc1nc(-c2cccnc2)nc2ccc(-c3ccc(F)cc3F)cc12] |
| 1534 | 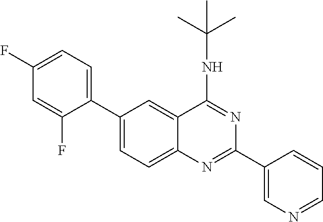 [SMILES: CC(C)(C)Nc1nc(-c2cccnc2)nc2ccc(-c3ccc(F)cc3F)cc12] |
| 1535 | 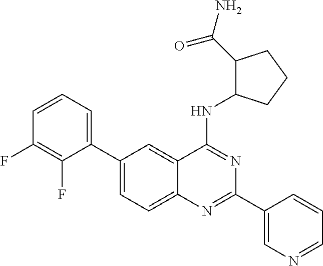 [SMILES: NC(=O)C1CCCC1Nc1nc(-c2cccnc2)nc2ccc(-c3cccc(F)c3F)cc12] |
| 1536 | 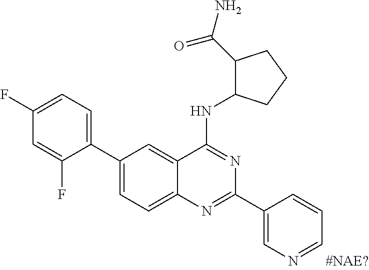 [SMILES: NC(=O)C1CCCC1Nc1nc(-c2cccnc2)nc2ccc(-c3ccc(F)cc3F)cc12] |
| 1537 | 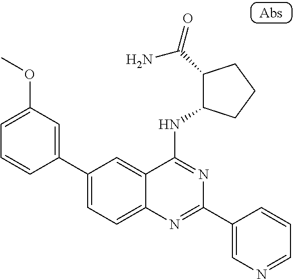 [SMILES: COc1cccc(-c2ccc3nc(-c4cccnc4)nc(N[C@H]4CCC[C@H]4C(N)=O)c3c2)c1] |
| 1538 | 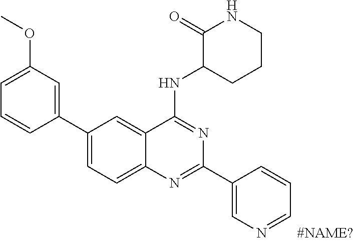 [SMILES: COc1cccc(-c2ccc3nc(-c4cccnc4)nc(NC4CCCNC4=O)c3c2)c1] |
| 1539 | 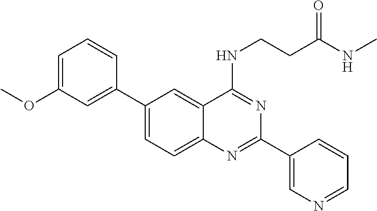 |
| 1540 | 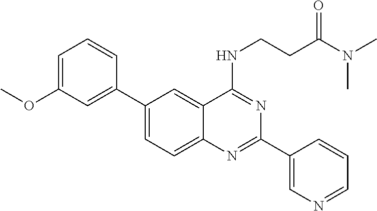 |
| 1541 | 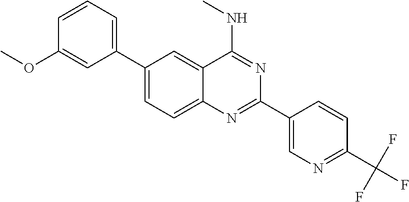 [SMILES: CNc1nc(-c2ccc(C(F)(F)F)nc2)nc2ccc(-c3cccc(OC)c3)cc12] |
| 1542 | 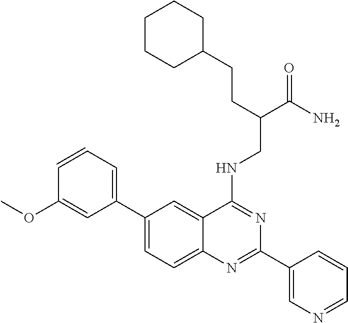 [SMILES: COc1cccc(-c2ccc3nc(-c4cccnc4)nc(NCC(CCC4CCCCC4)C(N)=O)c3c2)c1] |
| 1543 | 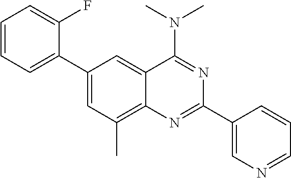 [SMILES: Cc1cc(-c2ccccc2F)cc2c(N(C)C)nc(-c3cccnc3)nc12] |
| 1544 | 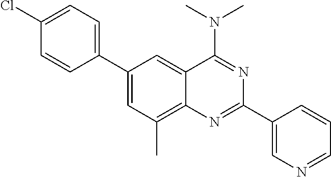 [SMILES: Cc1cc(-c2ccc(Cl)cc2)cc2c(N(C)C)nc(-c3cccnc3)nc12] |
| 1545 | 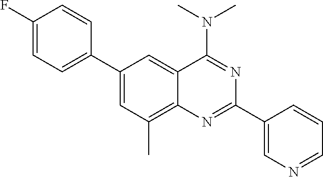 [SMILES: Cc1cc(-c2ccc(F)cc2)cc2c(N(C)C)nc(-c3cccnc3)nc12] |
| 1546 | 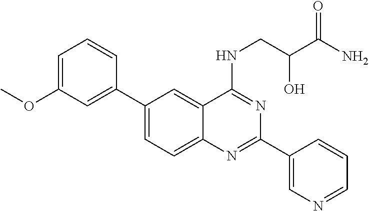 [SMILES: COc1cccc(-c2ccc3nc(-c4cccnc4)nc(NCC(O)C(N)=O)c3c2)c1] |
| 1547 | 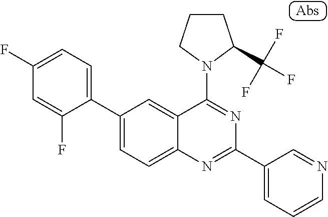 [SMILES: Fc1ccc(-c2ccc3nc(-c4cccnc4)nc(N4CCC[C@H]4C(F)(F)F)c3c2)c(F)c1] |
| 1548 | 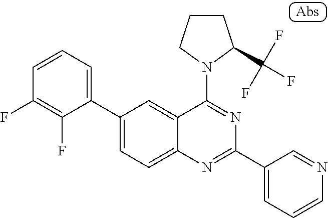 [SMILES: Fc1cccc(-c2ccc3nc(-c4cccnc4)nc(N4CCC[C@H]4C(F)(F)F)c3c2)c1F] |
| 1549 | 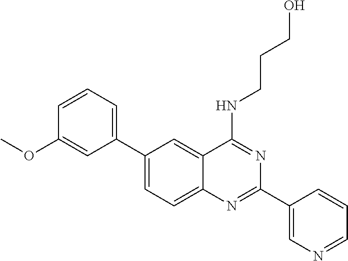 [SMILES: COc1cccc(-c2ccc3nc(-c4cccnc4)nc(NCCCO)c3c2)c1] |
| 1550 | 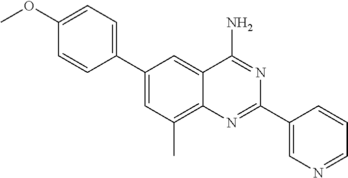 [SMILES: COc1ccc(-c2cc(C)c3nc(-c4cccnc4)nc(N)c3c2)cc1] |
| 1551 | 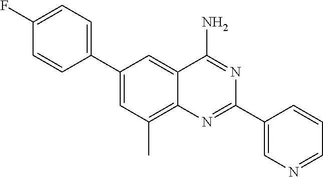 [SMILES: Cc1cc(-c2ccc(F)cc2)cc2c(N)nc(-c3cccnc3)nc12] |
| 1552 | 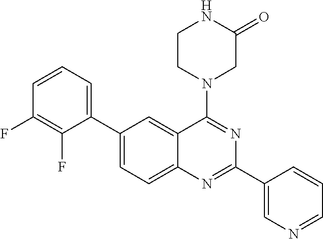 [SMILES: O=C1CN(c2nc(-c3cccnc3)nc3ccc(-c4cccc(F)c4F)cc23)CCN1] |
| 1553 | 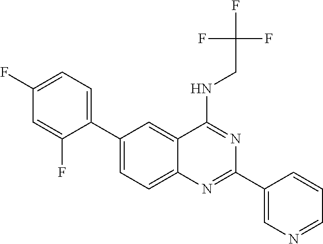 [SMILES: Fc1ccc(-c2ccc3nc(-c4cccnc4)nc(NCC(F)(F)F)c3c2)c(F)c1] |
| 1554 | 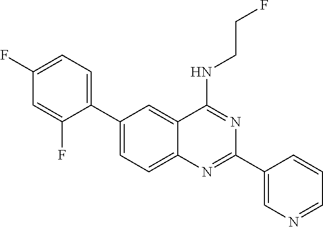 [SMILES: FCCNc1nc(-c2cccnc2)nc2ccc(-c3ccc(F)cc3F)cc12] |
| 1555 | 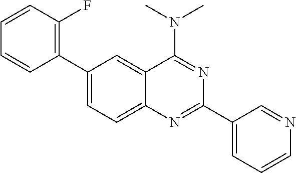 [SMILES: CN(C)c1nc(-c2cccnc2)nc2ccc(-c3ccccc3F)cc12] |
| 1556 | 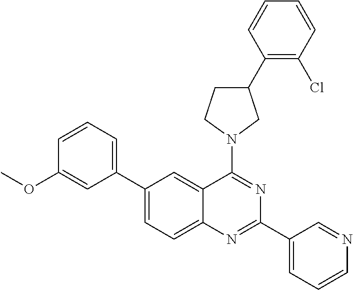 [SMILES: COc1cccc(-c2ccc3nc(-c4cccnc4)nc(N4CCC(c5ccccc5Cl)C4)c3c2)c1] |
| 1557 | 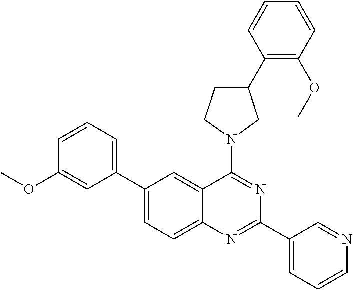 [SMILES: COc1cccc(-c2ccc3nc(-c4cccnc4)nc(N4CCC(c5ccccc5OC)C4)c3c2)c1] |
| 1558 | 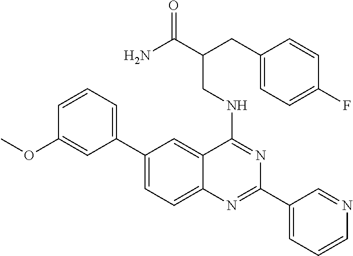 [SMILES: COc1cccc(-c2ccc3nc(-c4cccnc4)nc(NCC(Cc4ccc(F)cc4)C(N)=O)c3c2)c1] |
| 1559 | 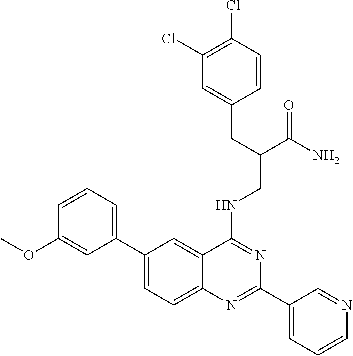 [SMILES: COc1cccc(-c2ccc3nc(-c4cccnc4)nc(NCC(Cc4ccc(Cl)c(Cl)c4)C(N)=O)c3c2)c1] |
| 1560 | 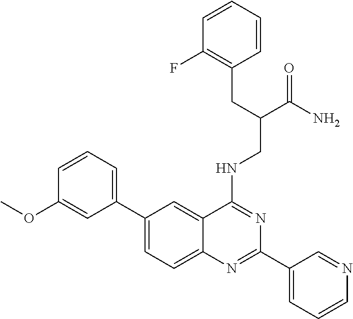 [SMILES: COc1cccc(-c2ccc3nc(-c4cccnc4)nc(NCC(Cc4ccccc4F)C(N)=O)c3c2)c1] |
| 1561 | 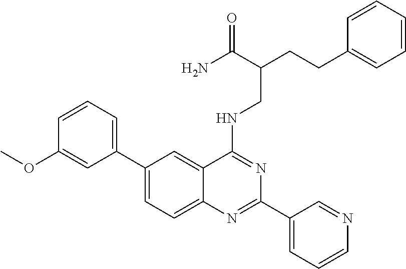 [SMILES: COc1cccc(-c2ccc3nc(-c4cccnc4)nc(NCC(CCc4ccccc4)C(N)=O)c3c2)c1] |
| 1562 | 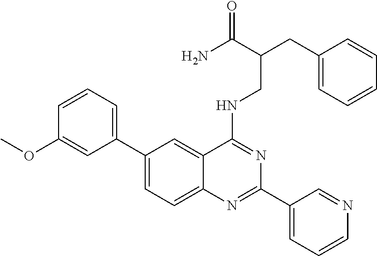 [SMILES: COc1cccc(-c2ccc3nc(-c4cccnc4)nc(NCC(Cc4ccccc4)C(N)=O)c3c2)c1] |
| 1563 | 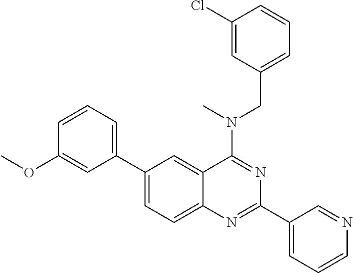 [SMILES: COc1cccc(-c2ccc3nc(-c4cccnc4)nc(N(C)Cc4cccc(Cl)c4)c3c2)c1] |
| 1564 | 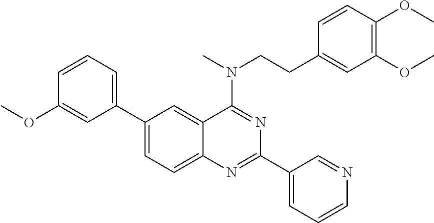 |
| 1565 | 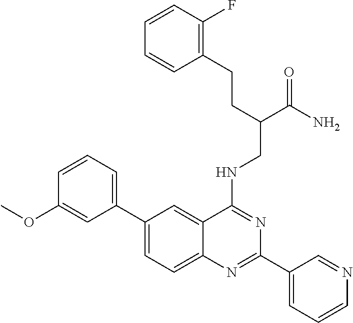 [SMILES: COc1cccc(-c2ccc3nc(-c4cccnc4)nc(NCC(CCc4ccccc4F)C(N)=O)c3c2)c1] |
| 1566 | 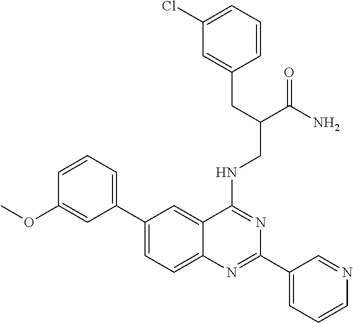 [SMILES: COc1cccc(-c2ccc3nc(-c4cccnc4)nc(NCC(Cc4cccc(Cl)c4)C(N)=O)c3c2)c1] |
| 1567 | 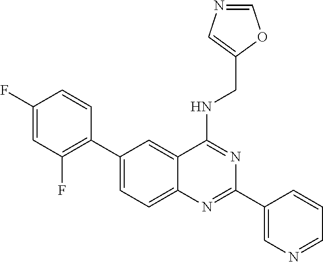 [SMILES: Fc1ccc(-c2ccc3nc(-c4cccnc4)nc(NCc4cnco4)c3c2)c(F)c1] |
| 1568 | 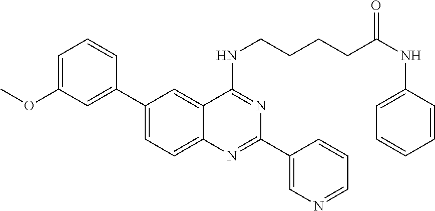 [SMILES: COc1cccc(-c2ccc3nc(-c4cccnc4)nc(NCCCCC(=O)Nc4ccccc4)c3c2)c1] |
| 1569 | 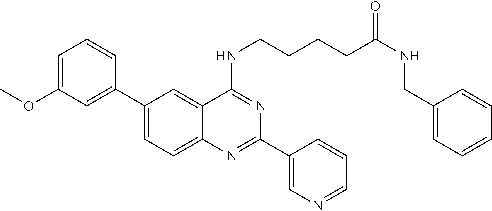 [SMILES: COc1cccc(-c2ccc3nc(-c4cccnc4)nc(NCCCCC(=O)NCc4ccccc4)c3c2)c1] |
| 1570 | 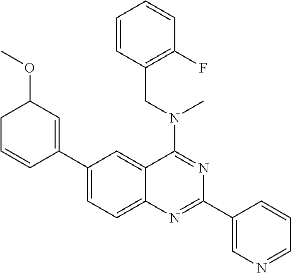 [SMILES: COC1C=C(c2ccc3nc(-c4cccnc4)nc(N(C)Cc4ccccc4F)c3c2)C=CC1] |
| 1571 | 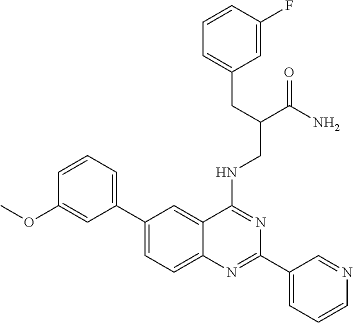 [SMILES: COc1cccc(-c2ccc3nc(-c4cccnc4)nc(NCC(Cc4cccc(F)c4)C(N)=O)c3c2)c1] |
| 1572 | 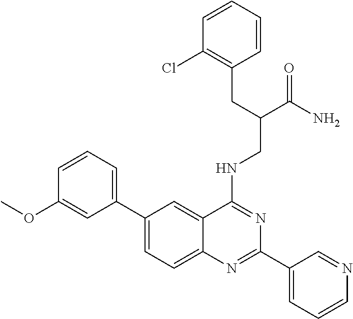 [SMILES: COc1cccc(-c2ccc3nc(-c4cccnc4)nc(NCC(Cc4ccccc4Cl)C(N)=O)c3c2)c1] |
| 1573 | 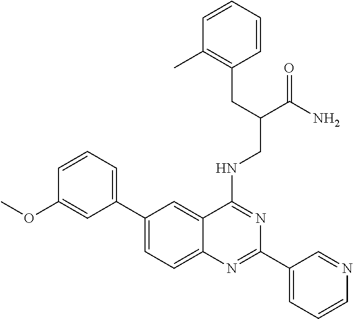 [SMILES: COc1cccc(-c2ccc3nc(-c4cccnc4)nc(NCC(Cc4ccccc4C)C(N)=O)c3c2)c1] |
| 1574 | 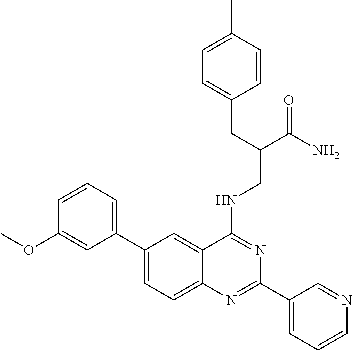 [SMILES: COc1cccc(-c2ccc3nc(-c4cccnc4)nc(NCC(Cc4ccc(C)cc4)C(N)=O)c3c2)c1] |
| 1575 | 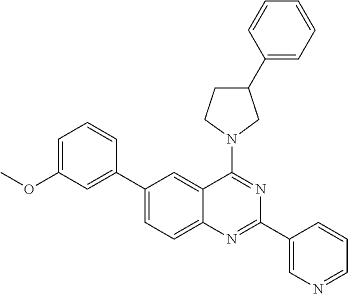 [SMILES: COc1cccc(-c2ccc3nc(-c4cccnc4)nc(N4CCC(c5ccccc5)C4)c3c2)c1] |
| 1576 | 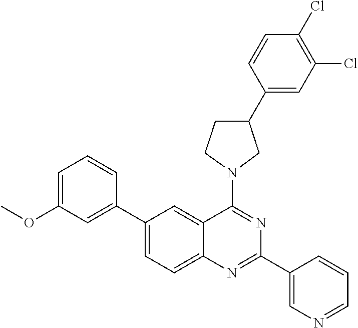 [SMILES: COc1cccc(-c2ccc3nc(-c4cccnc4)nc(N4CCC(c5ccc(Cl)c(Cl)c5)C4)c3c2)c1] |
| 1577 | 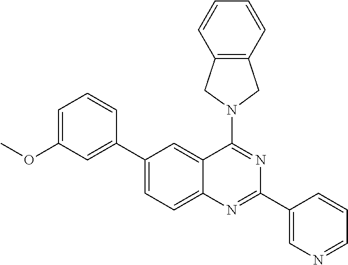 [SMILES: COc1cccc(-c2ccc3nc(-c4cccnc4)nc(N4Cc5ccccc5C4)c3c2)c1] |
| 1578 | 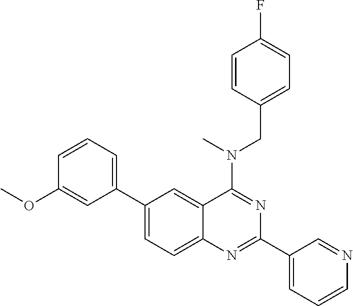 [SMILES: COc1cccc(-c2ccc3nc(-c4cccnc4)nc(N(C)Cc4ccc(F)cc4)c3c2)c1] |
| 1579 | 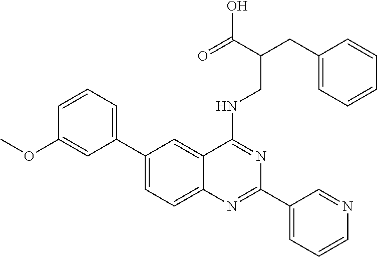 [SMILES: COc1cccc(-c2ccc3nc(-c4cccnc4)nc(NCC(Cc4ccccc4)C(=O)O)c3c2)c1] |
| 1580 | 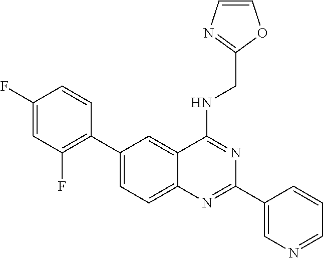 [SMILES: Fc1ccc(-c2ccc3nc(-c4cccnc4)nc(NCc4ncco4)c3c2)c(F)c1] |
| 1581 | 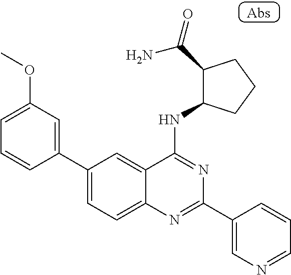 [SMILES: COc1cccc(-c2ccc3nc(-c4cccnc4)nc(N[C@@H]4CCC[C@@H]4C(N)=O)c3c2)c1] |
| 1583 | 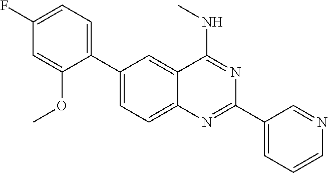 [SMILES: CNc1nc(-c2cccnc2)nc2ccc(-c3ccc(F)cc3OC)cc12] |
| 1584 | 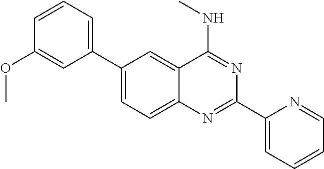 [SMILES: CNc1nc(-c2ccccn2)nc2ccc(-c3cccc(OC)c3)cc12] |
| 1585 | 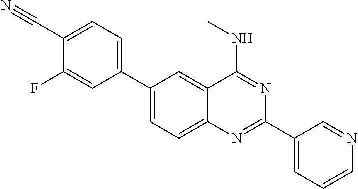 [SMILES: CNc1nc(-c2cccnc2)nc2ccc(-c3ccc(C#N)c(F)c3)cc12] |
| 1586 | 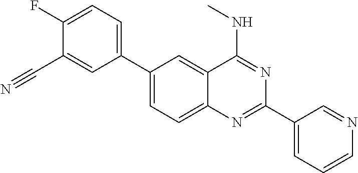 [SMILES: CNc1nc(-c2cccnc2)nc2ccc(-c3ccc(F)c(C#N)c3)cc12] |
| 1587 | 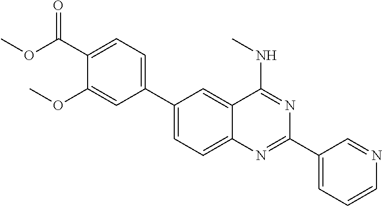 [SMILES: CNc1nc(-c2cccnc2)nc2ccc(-c3ccc(C(=O)OC)c(OC)c3)cc12] |
| 1588 | 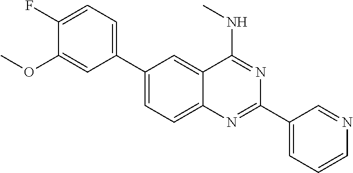 [SMILES: CNc1nc(-c2cccnc2)nc2ccc(-c3ccc(F)c(OC)c3)cc12] |
| 1589 | 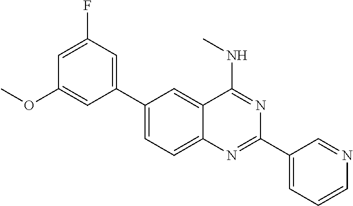 [SMILES: CNc1nc(-c2cccnc2)nc2ccc(-c3cc(F)cc(OC)c3)cc12] |
| 1590 | 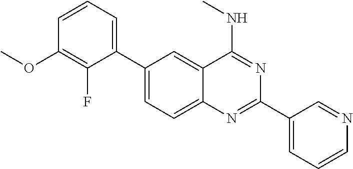 [SMILES: CNc1nc(-c2cccnc2)nc2ccc(-c3cccc(OC)c3F)cc12] |
| 1591 | 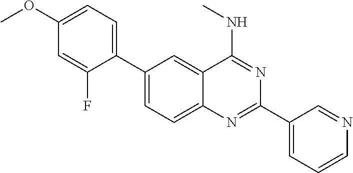 [SMILES: CNc1nc(-c2cccnc2)nc2ccc(-c3ccc(OC)cc3F)cc12] |
| 1592 | 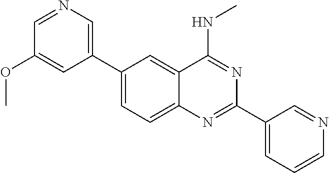 [SMILES: CNc1nc(-c2cccnc2)nc2ccc(-c3cncc(OC)c3)cc12] |
| 1593 | 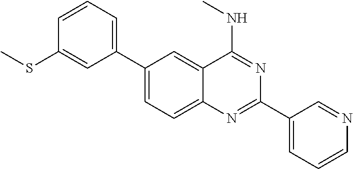 [SMILES: CNc1nc(-c2cccnc2)nc2ccc(-c3cccc(SC)c3)cc12] |
| 1594 | 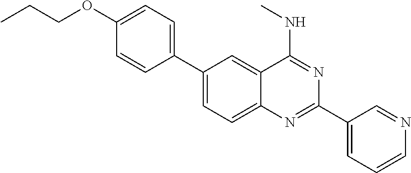 [SMILES: CCCOc1ccc(-c2ccc3nc(-c4cccnc4)nc(NC)c3c2)cc1] |
| 1595 | 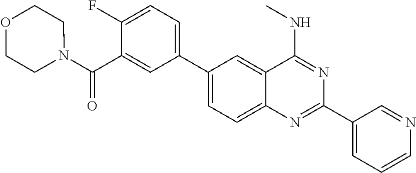 [SMILES: CNc1nc(-c2cccnc2)nc2ccc(-c3ccc(F)c(C(=O)N4CCOCC4)c3)cc12] |
| 1596 | 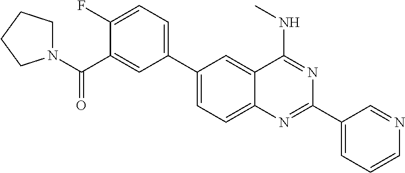 [SMILES: CNc1nc(-c2cccnc2)nc2ccc(-c3ccc(F)c(C(=O)N4CCCC4)c3)cc12] |
| 1597 | 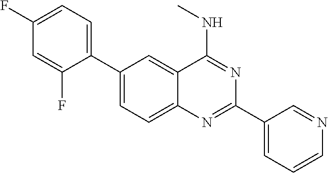 [SMILES: CNc1nc(-c2cccnc2)nc2ccc(-c3ccc(F)cc3F)cc12] |
| 1598 | 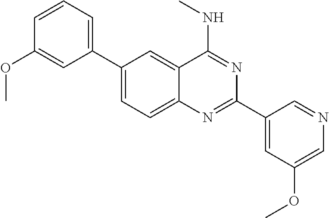 [SMILES: CNc1nc(-c2cncc(OC)c2)nc2ccc(-c3cccc(OC)c3)cc12] |
| 1599 | 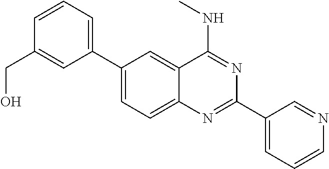 [SMILES: CNc1nc(-c2cccnc2)nc2ccc(-c3cccc(CO)c3)cc12] |
| 1600 | 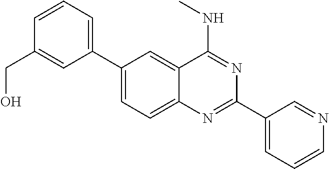 [SMILES: CNc1nc(-c2cccnc2)nc2ccc(-c3cccc(CO)c3)cc12] |
| 1601 | 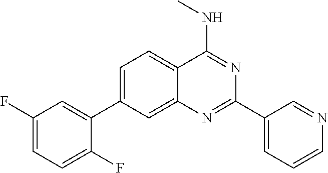 [SMILES: CNc1nc(-c2cccnc2)nc2cc(-c3cc(F)ccc3F)ccc12] |
| 1602 | 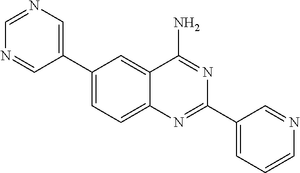 [SMILES: Nc1nc(-c2cccnc2)nc2ccc(-c3cncnc3)cc12] |
| 1603 | 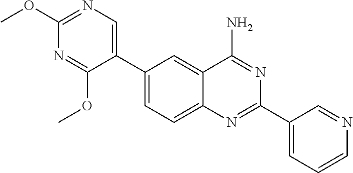 [SMILES: COc1ncc(-c2ccc3nc(-c4cccnc4)nc(N)c3c2)c(OC)n1] |
| 1604 | 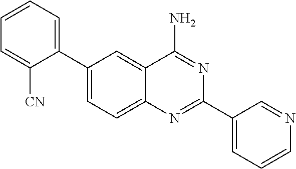 [SMILES: N#Cc1ccccc1-c1ccc2nc(-c3cccnc3)nc(N)c2c1] |
| 1605 | 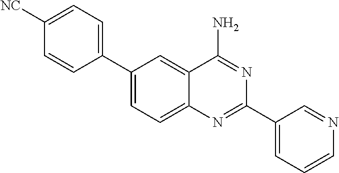 [SMILES: N#Cc1ccc(-c2ccc3nc(-c4cccnc4)nc(N)c3c2)cc1] |
| 1606 | 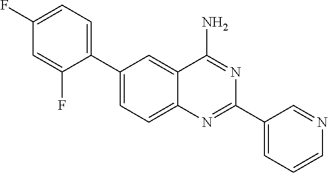 [SMILES: Nc1nc(-c2cccnc2)nc2ccc(-c3ccc(F)cc3F)cc12] |
| 1607 | 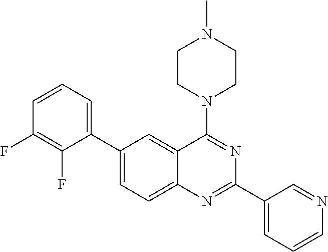 [SMILES: CN1CCN(c2nc(-c3cccnc3)nc3ccc(-c4cccc(F)c4F)cc23)CC1] |
| 1608 | 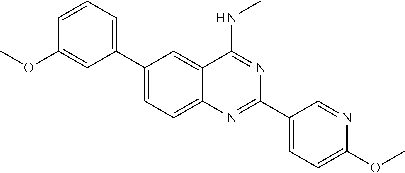 |
| 1609 | 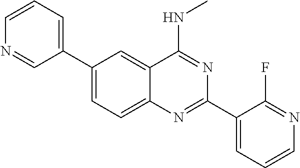 [SMILES: CNc1nc(-c2cccnc2F)nc2ccc(-c3cccnc3)cc12] |
| 1610 | 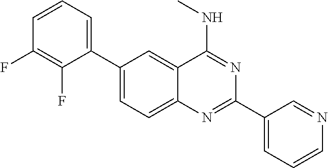 [SMILES: CNc1nc(-c2cccnc2)nc2ccc(-c3cccc(F)c3F)cc12] |
| 1611 | 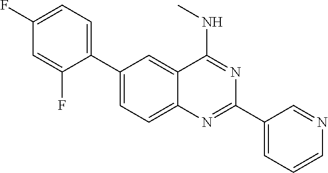 [SMILES: CNc1nc(-c2cccnc2)nc2ccc(-c3ccc(F)cc3F)cc12] |
| 1612 | 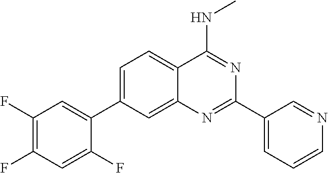 [SMILES: CNc1nc(-c2cccnc2)nc2cc(-c3cc(F)c(F)cc3F)ccc12] |
| 1613 | 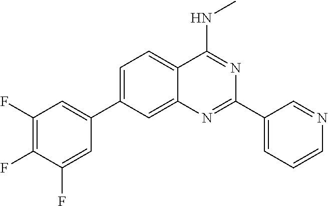 [SMILES: CNc1nc(-c2cccnc2)nc2cc(-c3cc(F)c(F)c(F)c3)ccc12] |
| 1614 | 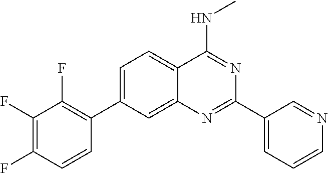 [SMILES: CNc1nc(-c2cccnc2)nc2cc(-c3ccc(F)c(F)c3F)ccc12] |
| 1615 | 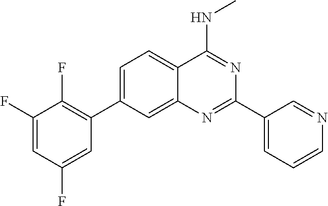 [SMILES: CNc1nc(-c2cccnc2)nc2cc(-c3cc(F)cc(F)c3F)ccc12] |
| 1760 | 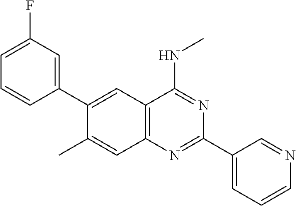 [SMILES: CNc1nc(-c2cccnc2)nc2cc(C)c(-c3cccc(F)c3)cc12] |
| 1616 | 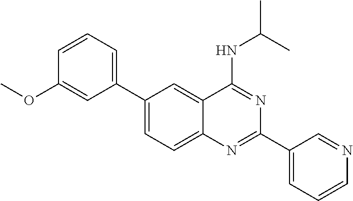 [SMILES: COc1cccc(-c2ccc3nc(-c4cccnc4)nc(NC(C)C)c3c2)c1] |
| 1617 | 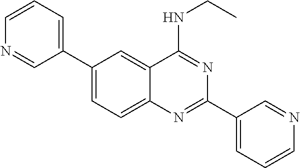 [SMILES: CCNc1nc(-c2cccnc2)nc2ccc(-c3cccnc3)cc12] |
| 1618 | 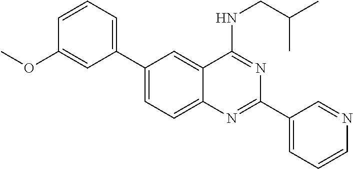 [SMILES: COc1cccc(-c2ccc3nc(-c4cccnc4)nc(NCC(C)C)c3c2)c1] |
| 1619 | 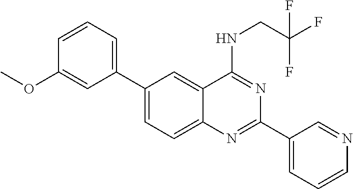 [SMILES: COc1cccc(-c2ccc3nc(-c4cccnc4)nc(NCC(F)(F)F)c3c2)c1] |
| 1620 | 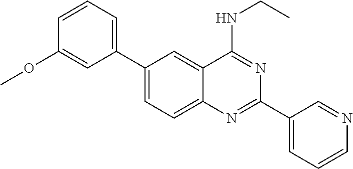 [SMILES: CCNc1nc(-c2cccnc2)nc2ccc(-c3cccc(OC)c3)cc12] |
| 1621 | 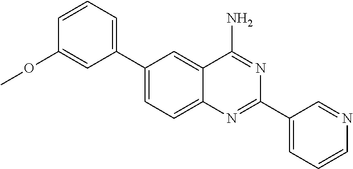 [SMILES: COc1cccc(-c2ccc3nc(-c4cccnc4)nc(N)c3c2)c1] |
| 1622 | 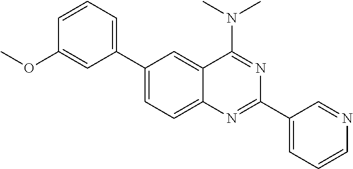 [SMILES: COc1cccc(-c2ccc3nc(-c4cccnc4)nc(N(C)C)c3c2)c1] |
| 1623 | 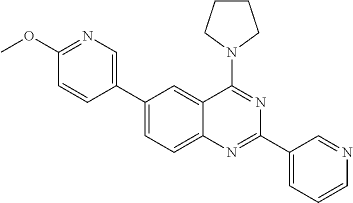 [SMILES: COc1ccc(-c2ccc3nc(-c4cccnc4)nc(N4CCCC4)c3c2)cn1] |
| 1624 | 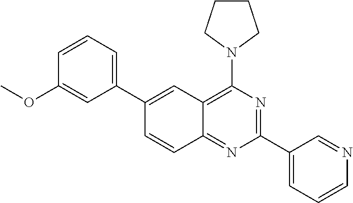 [SMILES: COc1cccc(-c2ccc3nc(-c4cccnc4)nc(N4CCCC4)c3c2)c1] |
| 1625 | 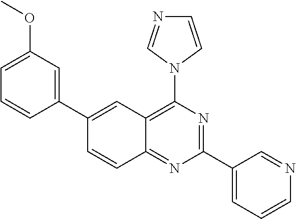 [SMILES: COc1cccc(-c2ccc3nc(-c4cccnc4)nc(-n4ccnc4)c3c2)c1] |
| 1626 | 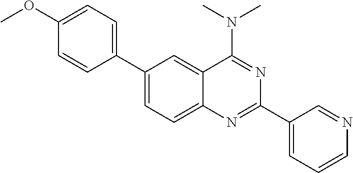 [SMILES: COc1ccc(-c2ccc3nc(-c4cccnc4)nc(N(C)C)c3c2)cc1] |
| 1627 | 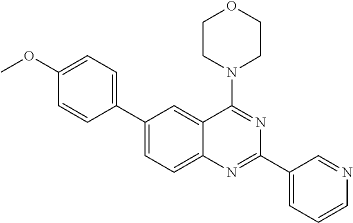 [SMILES: COc1ccc(-c2ccc3nc(-c4cccnc4)nc(N4CCOCC4)c3c2)cc1] |
| 1628 | 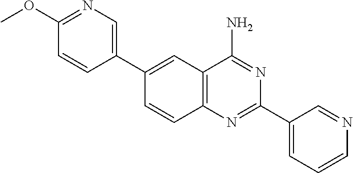 [SMILES: COc1ccc(-c2ccc3nc(-c4cccnc4)nc(N)c3c2)cn1] |
| 1629 | 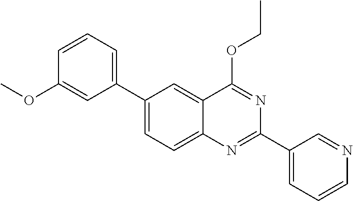 [SMILES: CCOc1nc(-c2cccnc2)nc2ccc(-c3cccc(OC)c3)cc12] |
| 1630 | 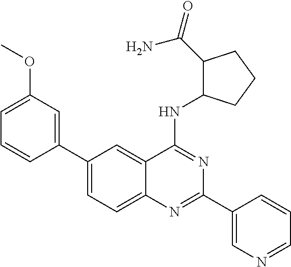 [SMILES: COc1cccc(-c2ccc3nc(-c4cccnc4)nc(NC4CCCC4C(N)=O)c3c2)c1] |
| 1631 | 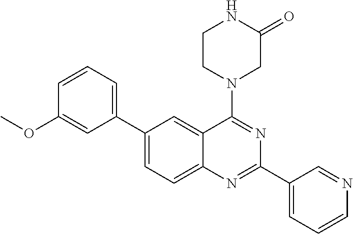 [SMILES: COc1cccc(-c2ccc3nc(-c4cccnc4)nc(N4CCNC(=O)C4)c3c2)c1] |
| 1632 | 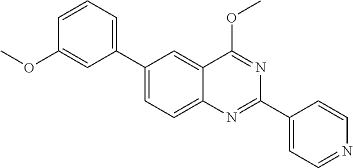 [SMILES: COc1cccc(-c2ccc3nc(-c4ccncc4)nc(OC)c3c2)c1] |
| 1634 | 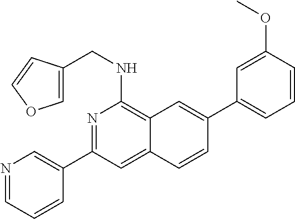 [SMILES: COc1cccc(-c2ccc3cc(-c4cccnc4)nc(NCc4ccoc4)c3c2)c1] |
| 1635 | 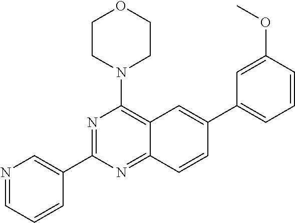 [SMILES: COc1cccc(-c2ccc3nc(-c4cccnc4)nc(N4CCOCC4)c3c2)c1] |
| 1636 | 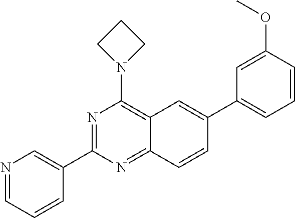 [SMILES: COc1cccc(-c2ccc3nc(-c4cccnc4)nc(N4CCC4)c3c2)c1] |
| 1637 | 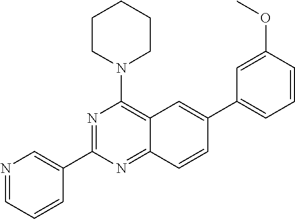 [SMILES: COc1cccc(-c2ccc3nc(-c4cccnc4)nc(N4CCCCC4)c3c2)c1] |
| 1638 | 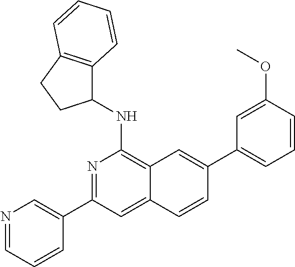 [SMILES: COc1cccc(-c2ccc3cc(-c4cccnc4)nc(NC4CCc5ccccc54)c3c2)c1] |
| 1639 | 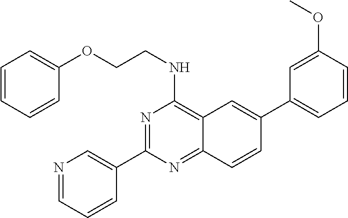 [SMILES: COc1cccc(-c2ccc3nc(-c4cccnc4)nc(NCCOc4ccccc4)c3c2)c1] |
| 1633 | 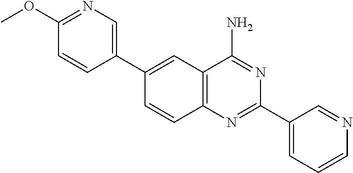 [SMILES: COc1ccc(-c2ccc3nc(-c4cccnc4)nc(N)c3c2)cn1] |
| 1640 | 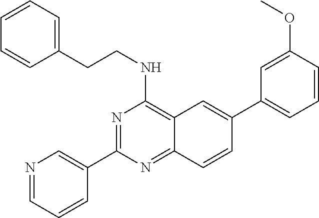 [SMILES: COc1cccc(-c2ccc3nc(-c4cccnc4)nc(NCCc4ccccc4)c3c2)c1] |
| 1641 | 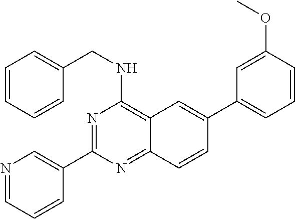 [SMILES: COc1cccc(-c2ccc3nc(-c4cccnc4)nc(NCc4ccccc4)c3c2)c1] |
| 1642 | 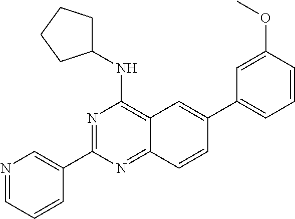 [SMILES: COc1cccc(-c2ccc3nc(-c4cccnc4)nc(NC4CCCC4)c3c2)c1] |
| 1643 | 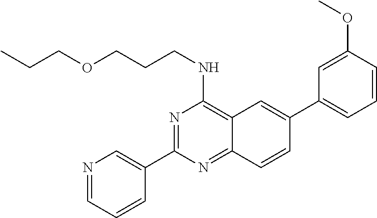 [SMILES: CCCOCCCNc1nc(-c2cccnc2)nc2ccc(-c3cccc(OC)c3)cc12] |
| 1644 | 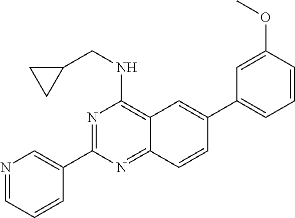 [SMILES: COc1cccc(-c2ccc3nc(-c4cccnc4)nc(NCC4CC4)c3c2)c1] |
| 1645 | 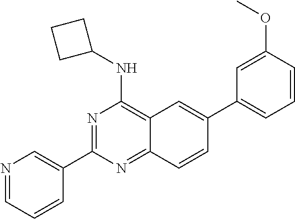 [SMILES: COc1cccc(-c2ccc3nc(-c4cccnc4)nc(NC4CCC4)c3c2)c1] |
| 1646 | 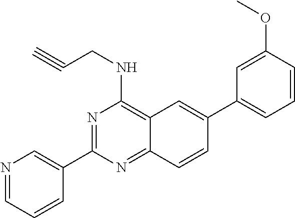 [SMILES: C#CCNc1nc(-c2cccnc2)nc2ccc(-c3cccc(OC)c3)cc12] |
| 1647 | 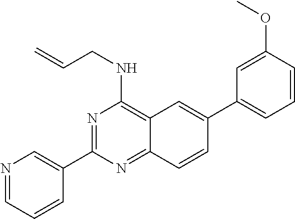 [SMILES: C=CCNc1nc(-c2cccnc2)nc2ccc(-c3cccc(OC)c3)cc12] |
| 1648 | 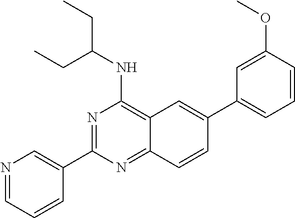 [SMILES: CCC(CC)Nc1nc(-c2cccnc2)nc2ccc(-c3cccc(OC)c3)cc12] |
| 1649 | 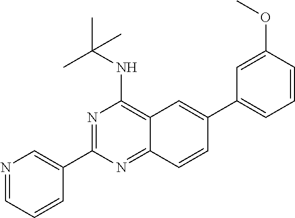 [SMILES: COc1cccc(-c2ccc3nc(-c4cccnc4)nc(NC(C)(C)C)c3c2)c1] |
| 1650 | 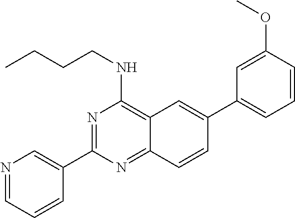 [SMILES: CCCCNc1nc(-c2cccnc2)nc2ccc(-c3cccc(OC)c3)cc12] |
| 1651 | 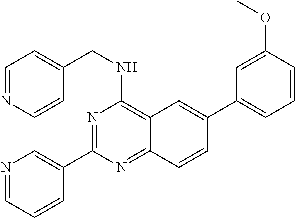 [SMILES: COc1cccc(-c2ccc3nc(-c4cccnc4)nc(NCc4ccncc4)c3c2)c1] |
| 1652 | 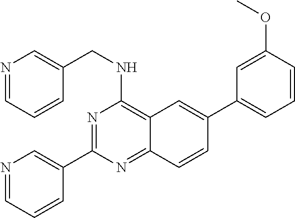 [SMILES: COc1cccc(-c2ccc3nc(-c4cccnc4)nc(NCc4cccnc4)c3c2)c1] |
| 1653 | 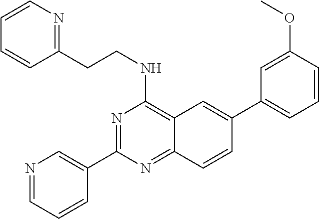 [SMILES: COc1cccc(-c2ccc3nc(-c4cccnc4)nc(NCCc4ccccn4)c3c2)c1] |
| 1654 | 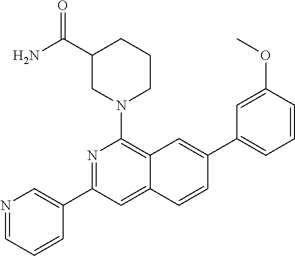 [SMILES: COc1cccc(-c2ccc3cc(-c4cccnc4)nc(N4CCCC(C(N)=O)C4)c3c2)c1] |
| 1655 | 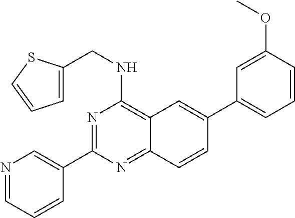 [SMILES: COc1cccc(-c2ccc3nc(-c4cccnc4)nc(NCc4cccs4)c3c2)c1] |
| 1656 | 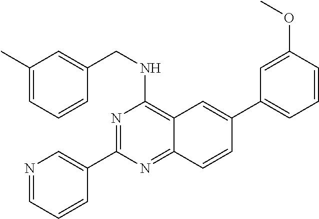 [SMILES: COc1cccc(-c2ccc3nc(-c4cccnc4)nc(NCc4cccc(C)c4)c3c2)c1] |
| 1657 | 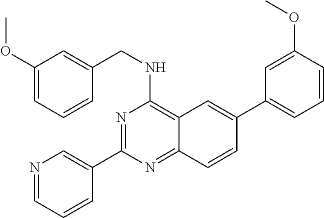 [SMILES: COc1cccc(CNc2nc(-c3cccnc3)nc3ccc(-c4cccc(OC)c4)cc23)c1] |
| 1658 | 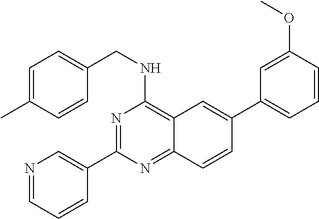 [SMILES: COc1cccc(-c2ccc3nc(-c4cccnc4)nc(NCc4ccc(C)cc4)c3c2)c1] |
| 1659 | 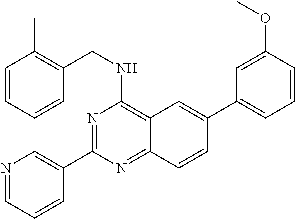 [SMILES: COc1cccc(-c2ccc3nc(-c4cccnc4)nc(NCc4ccccc4C)c3c2)c1] |
| 1660 | 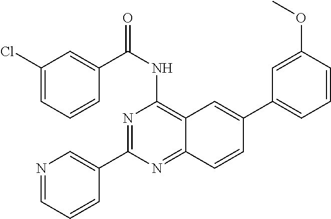 [SMILES: COc1cccc(-c2ccc3nc(-c4cccnc4)nc(NC(=O)c4cccc(Cl)c4)c3c2)c1] |
| 1661 | 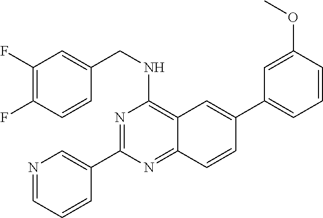 [SMILES: COc1cccc(-c2ccc3nc(-c4cccnc4)nc(NCc4ccc(F)c(F)c4)c3c2)c1] |
| 1662 | 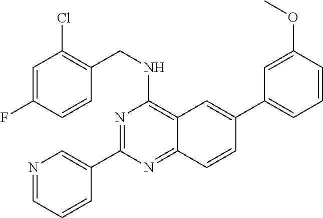 [SMILES: COc1cccc(-c2ccc3nc(-c4cccnc4)nc(NCc4ccc(F)cc4Cl)c3c2)c1] |
| 1663 | 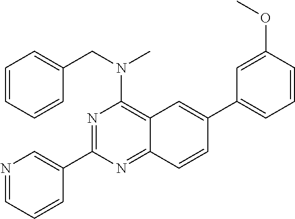 [SMILES: COc1cccc(-c2ccc3nc(-c4cccnc4)nc(N(C)Cc4ccccc4)c3c2)c1] |
| 1664 | 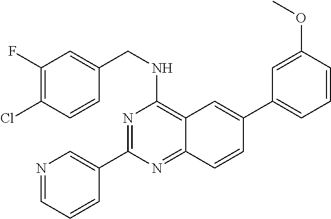 [SMILES: COc1cccc(-c2ccc3nc(-c4cccnc4)nc(NCc4ccc(Cl)c(F)c4)c3c2)c1] |
| 1665 | 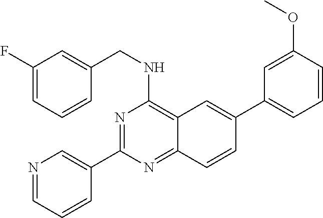 [SMILES: COc1cccc(-c2ccc3nc(-c4cccnc4)nc(NCc4cccc(F)c4)c3c2)c1] |
| 1666 | 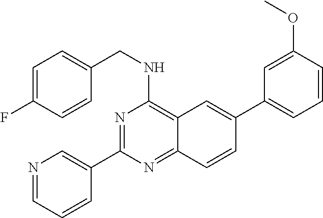 [SMILES: COc1cccc(-c2ccc3nc(-c4cccnc4)nc(NCc4ccc(F)cc4)c3c2)c1] |
| 1667 | 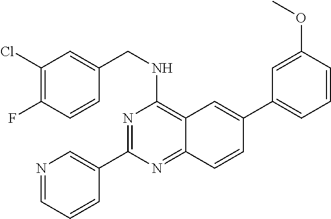 [SMILES: COc1cccc(-c2ccc3nc(-c4cccnc4)nc(NCc4ccc(F)c(Cl)c4)c3c2)c1] |
| 1668 | 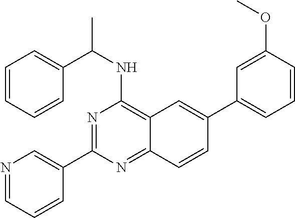 [SMILES: COc1cccc(-c2ccc3nc(-c4cccnc4)nc(NC(C)c4ccccc4)c3c2)c1] |
| 1669 | 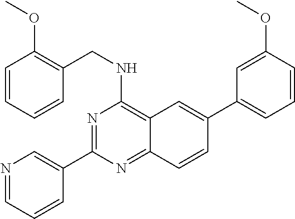 [SMILES: COc1cccc(-c2ccc3nc(-c4cccnc4)nc(NCc4ccccc4OC)c3c2)c1] |
| 1670 | 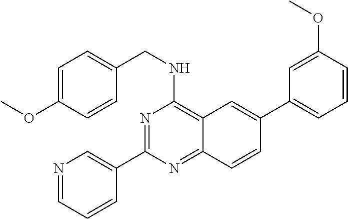 [SMILES: COc1ccc(CNc2nc(-c3cccnc3)nc3ccc(-c4cccc(OC)c4)cc23)cc1] |
| 1671 | 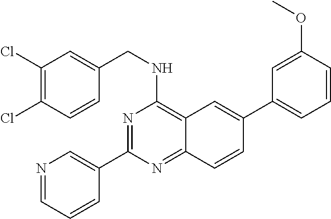 [SMILES: COc1cccc(-c2ccc3nc(-c4cccnc4)nc(NCc4ccc(Cl)c(Cl)c4)c3c2)c1] |
| 1672 | 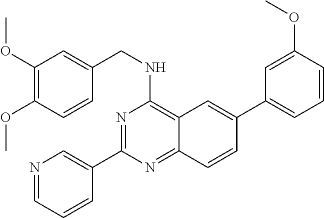 [SMILES: COc1cccc(-c2ccc3nc(-c4cccnc4)nc(NCc4ccc(OC)c(OC)c4)c3c2)c1] |
| 1673 | 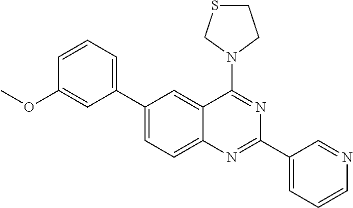 [SMILES: COc1cccc(-c2ccc3nc(-c4cccnc4)nc(N4CCSC4)c3c2)c1] |
| 1674 | 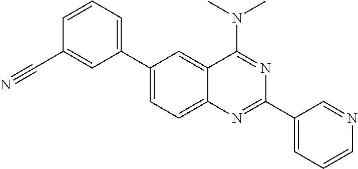 [SMILES: CN(C)c1nc(-c2cccnc2)nc2ccc(-c3cccc(C#N)c3)cc12] |
| 1675 | 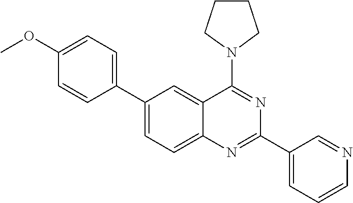 [SMILES: COc1ccc(-c2ccc3nc(-c4cccnc4)nc(N4CCCC4)c3c2)cc1] |
| 1676 | 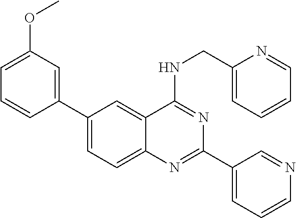 [SMILES: COc1cccc(-c2ccc3nc(-c4cccnc4)nc(NCc4ccccn4)c3c2)c1] |
| 1677 | 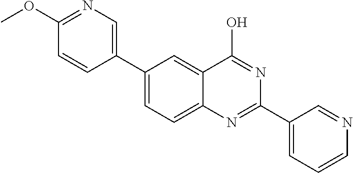 [SMILES: COc1ccc(-c2ccc3nc(-c4cccnc4)nc(O)c3c2)cn1] |
| 1678 | 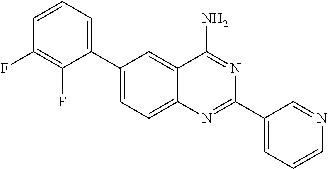 [SMILES: Nc1nc(-c2cccnc2)nc2ccc(-c3cccc(F)c3F)cc12] |
| 1679 | 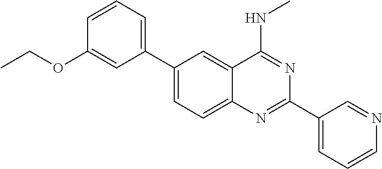 [SMILES: CCOc1cccc(-c2ccc3nc(-c4cccnc4)nc(NC)c3c2)c1] |
| 1680 | 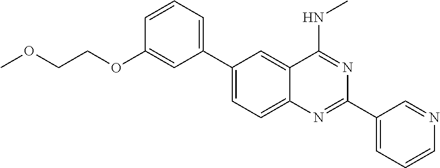 [SMILES: CNc1nc(-c2cccnc2)nc2ccc(-c3cccc(OCCOC)c3)cc12] |
| 1681 | 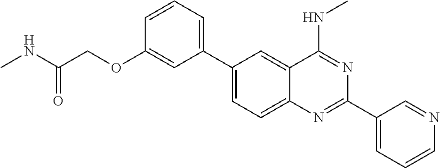 [SMILES: CNC(=O)COc1cccc(-c2ccc3nc(-c4cccnc4)nc(NC)c3c2)c1] |
| 1682 | 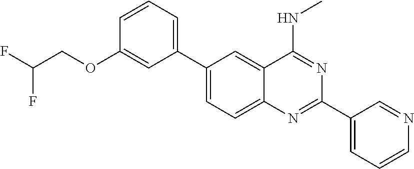 [SMILES: CNc1nc(-c2cccnc2)nc2ccc(-c3cccc(OCC(F)F)c3)cc12] |
| 1683 | 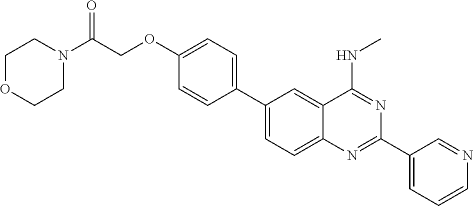 [SMILES: CNc1nc(-c2cccnc2)nc2ccc(-c3ccc(OCC(=O)N4CCOCC4)cc3)cc12] |
| 1684 | 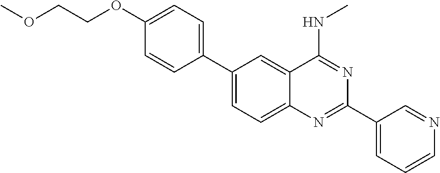 [SMILES: CNc1nc(-c2cccnc2)nc2ccc(-c3ccc(OCCOC)cc3)cc12] |
| 1685 | 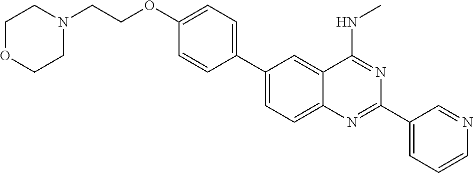 [SMILES: CNc1nc(-c2cccnc2)nc2ccc(-c3ccc(OCCN4CCOCC4)cc3)cc12] |
| 1686 | 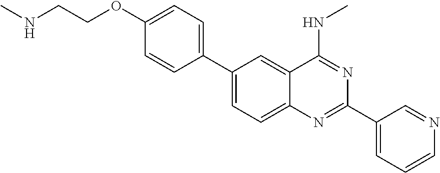 [SMILES: CNCCOc1ccc(-c2ccc3nc(-c4cccnc4)nc(NC)c3c2)cc1] |
| 1687 | 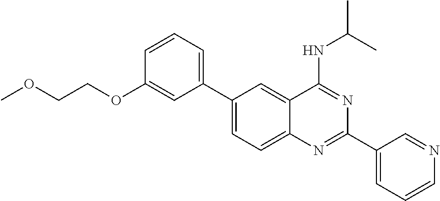 [SMILES: COCCOc1cccc(-c2ccc3nc(-c4cccnc4)nc(NC(C)C)c3c2)c1] |
| 1688 | 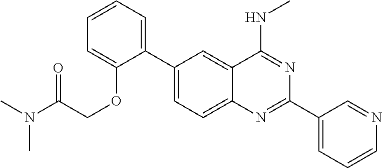 [SMILES: CNc1nc(-c2cccnc2)nc2ccc(-c3ccccc3OCC(=O)N(C)C)cc12] |
| 1689 | 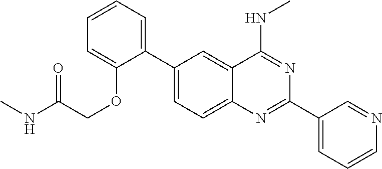 [SMILES: CNC(=O)COc1ccccc1-c1ccc2nc(-c3cccnc3)nc(NC)c2c1] |
| 1690 | 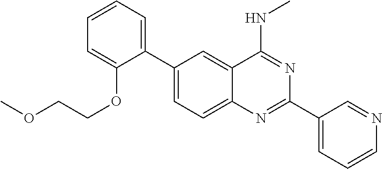 [SMILES: CNc1nc(-c2cccnc2)nc2ccc(-c3ccccc3OCCOC)cc12] |
| 1691 | 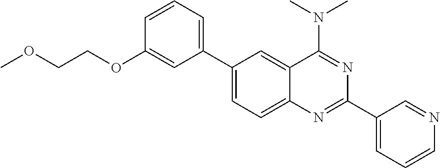 [SMILES: COCCOc1cccc(-c2ccc3nc(-c4cccnc4)nc(N(C)C)c3c2)c1] |
| 1692 | 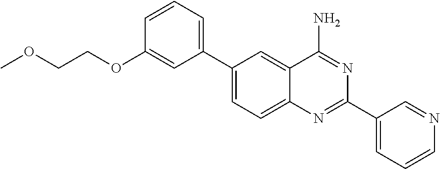 [SMILES: COCCOc1cccc(-c2ccc3nc(-c4cccnc4)nc(N)c3c2)c1] |
| 1693 | 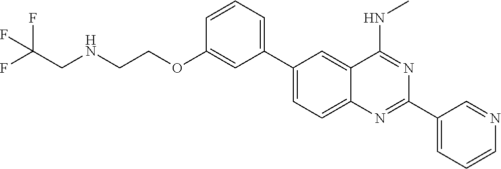 [SMILES: CNc1nc(-c2cccnc2)nc2ccc(-c3cccc(OCCNCC(F)(F)F)c3)cc12] |
| 1694 | 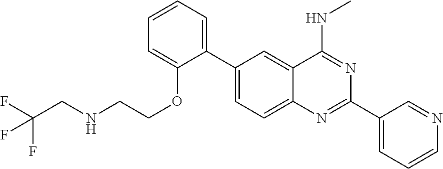 [SMILES: CNc1nc(-c2cccnc2)nc2ccc(-c3ccccc3OCCNCC(F)(F)F)cc12] |
| 1695 | 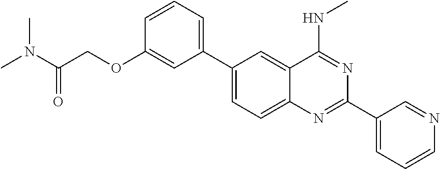 [SMILES: CNc1nc(-c2cccnc2)nc2ccc(-c3cccc(OCC(=O)N(C)C)c3)cc12] |
| 1697 | 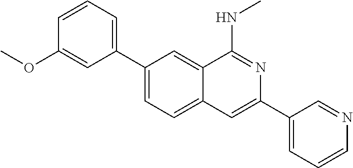 [SMILES: CNc1nc(-c2cccnc2)cc2ccc(-c3cccc(OC)c3)cc12] |
| 1698 | 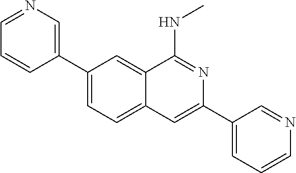 [SMILES: CNc1nc(-c2cccnc2)cc2ccc(-c3cccnc3)cc12] |
| 1699 | 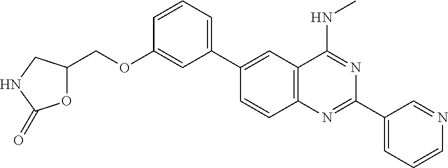 [SMILES: CNc1nc(-c2cccnc2)nc2ccc(-c3cccc(OCC4CNC(=O)O4)c3)cc12] |
| 1700 | 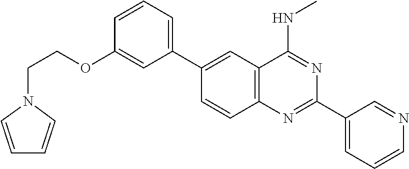 [SMILES: CNc1nc(-c2cccnc2)nc2ccc(-c3cccc(OCCn4cccc4)c3)cc12] |
| 1701 | 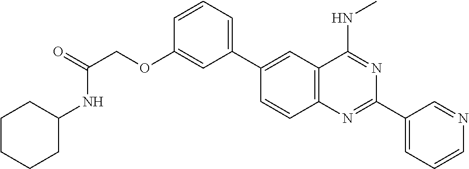 [SMILES: CNc1nc(-c2cccnc2)nc2ccc(-c3cccc(OCC(=O)NC4CCCCC4)c3)cc12] |
| 1702 | 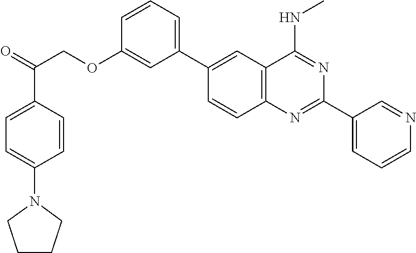 [SMILES: CNc1nc(-c2cccnc2)nc2ccc(-c3cccc(OCC(=O)c4ccc(N5CCCC5)cc4)c3)cc12] |
| 1703 | 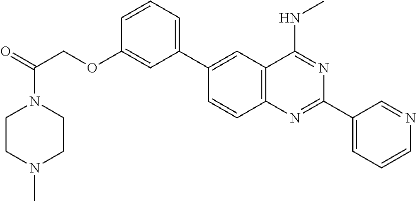 [SMILES: CNc1nc(-c2cccnc2)nc2ccc(-c3cccc(OCC(=O)N4CCN(C)CC4)c3)cc12] |
| 1704 | 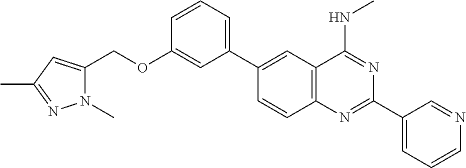 [SMILES: CNc1nc(-c2cccnc2)nc2ccc(-c3cccc(OCc4cc(C)nn4C)c3)cc12] |
| 1705 | 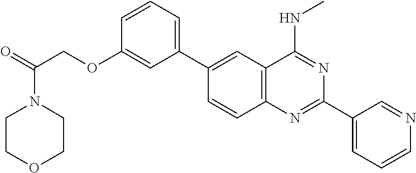 [SMILES: CNc1nc(-c2cccnc2)nc2ccc(-c3cccc(OCC(=O)N4CCOCC4)c3)cc12] |
| 1706 | 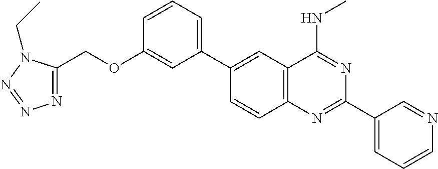 [SMILES: CCn1nnnc1COc1cccc(-c2ccc3nc(-c4cccnc4)nc(NC)c3c2)c1] |
| 1707 | 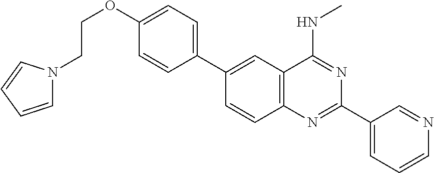 [SMILES: CNc1nc(-c2cccnc2)nc2ccc(-c3ccc(OCCn4cccc4)cc3)cc12] |
| 1708 | 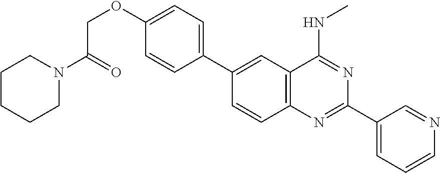 [SMILES: CNc1nc(-c2cccnc2)nc2ccc(-c3ccc(OCC(=O)N4CCCCC4)cc3)cc12] |
| 1709 | 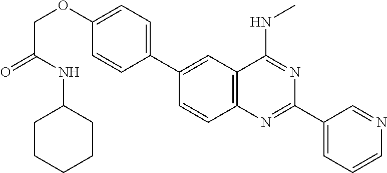 [SMILES: CNc1nc(-c2cccnc2)nc2ccc(-c3ccc(OCC(=O)NC4CCCCC4)cc3)cc12] |
| 1710 | 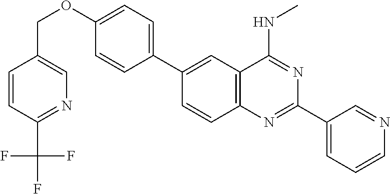 [SMILES: CNc1nc(-c2cccnc2)nc2ccc(-c3ccc(OCc4ccc(C(F)(F)F)nc4)cc3)cc12] |
| 1711 | 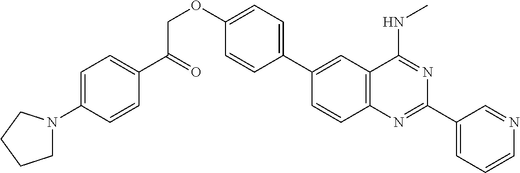 [SMILES: CNc1nc(-c2cccnc2)nc2ccc(-c3ccc(OCC(=O)c4ccc(N5CCCC5)cc4)cc3)cc12] |
| 1712 | 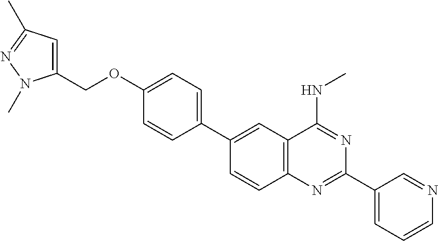 [SMILES: CNc1nc(-c2cccnc2)nc2ccc(-c3ccc(OCc4cc(C)nn4C)cc3)cc12] |
| 1713 | 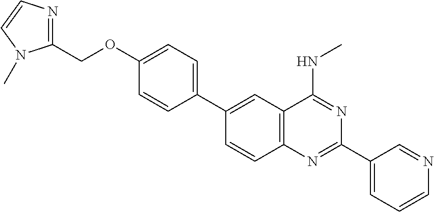 [SMILES: CNc1nc(-c2cccnc2)nc2ccc(-c3ccc(OCc4nccn4C)cc3)cc12] |
| 1714 | 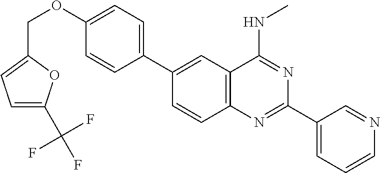 [SMILES: CNc1nc(-c2cccnc2)nc2ccc(-c3ccc(OCc4ccc(C(F)(F)F)o4)cc3)cc12] |
| 1715 | 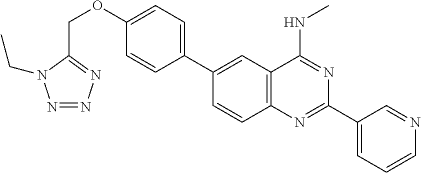 [SMILES: CCn1nnnc1COc1ccc(-c2ccc3nc(-c4cccnc4)nc(NC)c3c2)cc1] |
| 1716 | 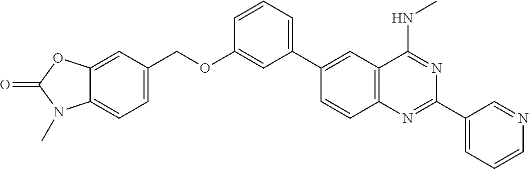 [SMILES: CNc1nc(-c2cccnc2)nc2ccc(-c3cccc(OCc4ccc5c(c4)oc(=O)n5C)c3)cc12] |
| 1717 | 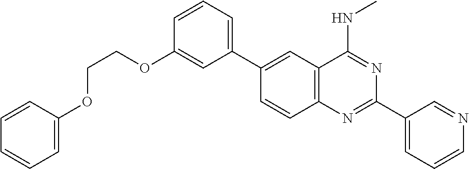 [SMILES: CNc1nc(-c2cccnc2)nc2ccc(-c3cccc(OCCOc4ccccc4)c3)cc12] |
| 1718 | 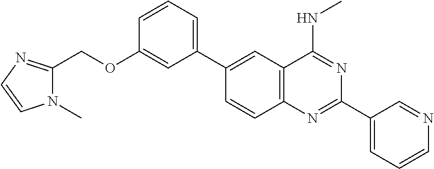 [SMILES: CNc1nc(-c2cccnc2)nc2ccc(-c3cccc(OCc4nccn4C)c3)cc12] |
| 1719 | 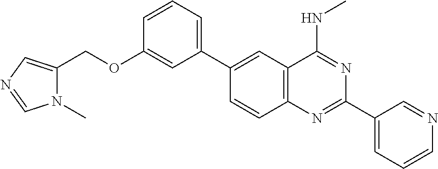 [SMILES: CNc1nc(-c2cccnc2)nc2ccc(-c3cccc(OCc4cncn4C)c3)cc12] |
| 1720 | 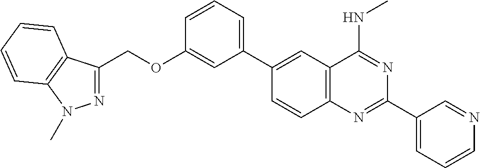 [SMILES: CNc1nc(-c2cccnc2)nc2ccc(-c3cccc(OCc4nn(C)c5ccccc45)c3)cc12] |
| 1721 | 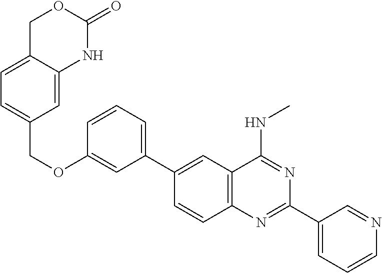 [SMILES: CNc1nc(-c2cccnc2)nc2ccc(-c3cccc(OCc4ccc5c(c4)NC(=O)OC5)c3)cc12] |
| 1722 | 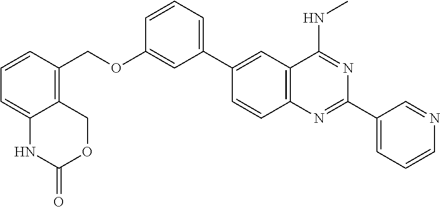 [SMILES: CNc1nc(-c2cccnc2)nc2ccc(-c3cccc(OCc4cccc5c4COC(=O)N5)c3)cc12] |
| 1723 | 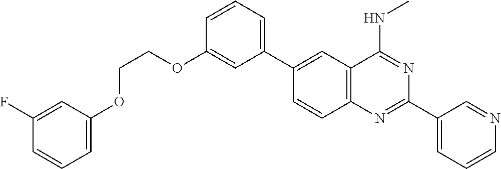 [SMILES: CNc1nc(-c2cccnc2)nc2ccc(-c3cccc(OCCOc4cccc(F)c4)c3)cc12] |
| 1724 | 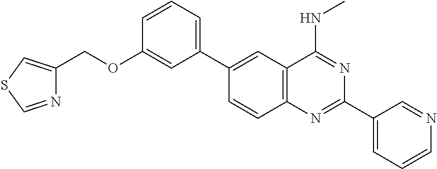 [SMILES: CNc1nc(-c2cccnc2)nc2ccc(-c3cccc(OCc4cscn4)c3)cc12] |
| 1725 | 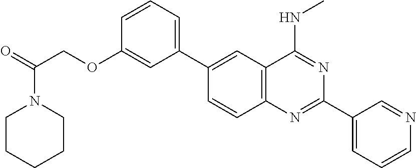 [SMILES: CNc1nc(-c2cccnc2)nc2ccc(-c3cccc(OCC(=O)N4CCCCC4)c3)cc12] |
| 1726 | 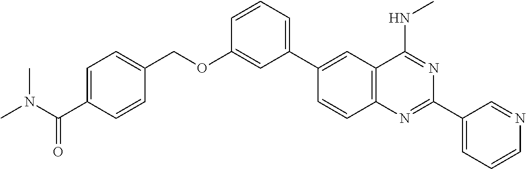 [SMILES: CNc1nc(-c2cccnc2)nc2ccc(-c3cccc(OCc4ccc(C(=O)N(C)C)cc4)c3)cc12] |
| 1727 | 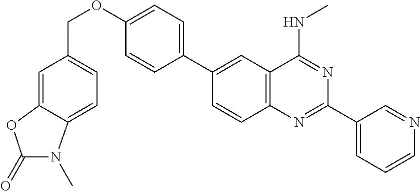 [SMILES: CNc1nc(-c2cccnc2)nc2ccc(-c3ccc(OCc4ccc5c(c4)oc(=O)n5C)cc3)cc12] |
| 1728 | 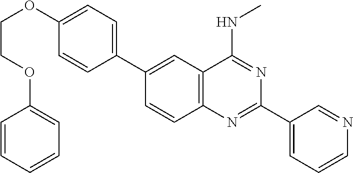 [SMILES: CNc1nc(-c2cccnc2)nc2ccc(-c3ccc(OCCOc4ccccc4)cc3)cc12] |
| 1729 | 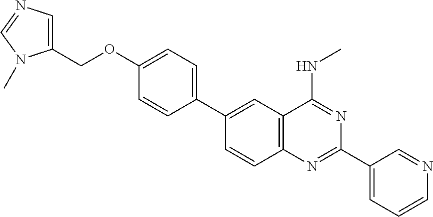 [SMILES: CNc1nc(-c2cccnc2)nc2ccc(-c3ccc(OCc4cncn4C)cc3)cc12] |
| 1730 | 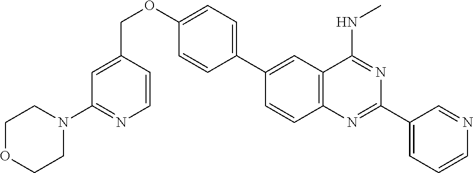 [SMILES: CNc1nc(-c2cccnc2)nc2ccc(-c3ccc(OCc4ccnc(N5CCOCC5)c4)cc3)cc12] |
| 1731 | 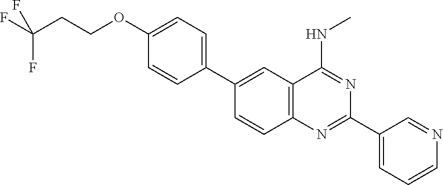 [SMILES: CNc1nc(-c2cccnc2)nc2ccc(-c3ccc(OCCC(F)(F)F)cc3)cc12] |
| 1732 | 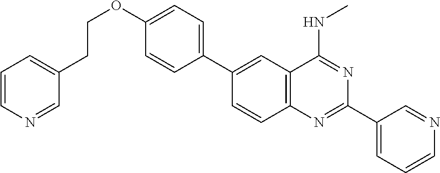 [SMILES: CNc1nc(-c2cccnc2)nc2ccc(-c3ccc(OCCc4cccnc4)cc3)cc12] |
| 1733 | 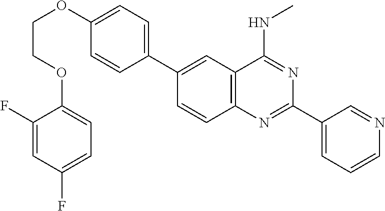 [SMILES: CNc1nc(-c2cccnc2)nc2ccc(-c3ccc(OCCOc4ccc(F)cc4F)cc3)cc12] |
| 1734 | 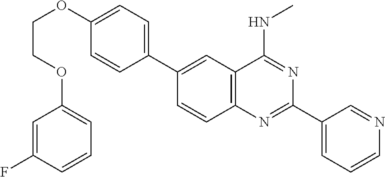 [SMILES: CNc1nc(-c2cccnc2)nc2ccc(-c3ccc(OCCOc4cccc(F)c4)cc3)cc12] |
| 1735 | 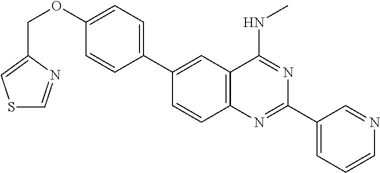 [SMILES: CNc1nc(-c2cccnc2)nc2ccc(-c3ccc(OCc4cscn4)cc3)cc12] |
| 1736 | 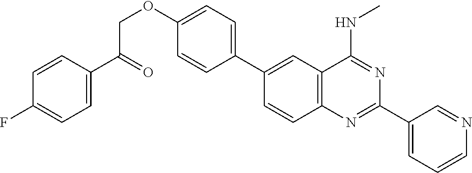 [SMILES: CNc1nc(-c2cccnc2)nc2ccc(-c3ccc(OCC(=O)c4ccc(F)cc4)cc3)cc12] |
| 1737 | 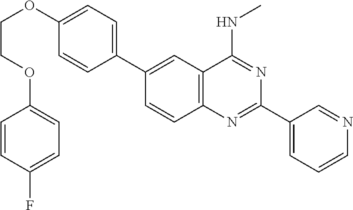 [SMILES: CNc1nc(-c2cccnc2)nc2ccc(-c3ccc(OCCOc4ccc(F)cc4)cc3)cc12] |
| 1738 | 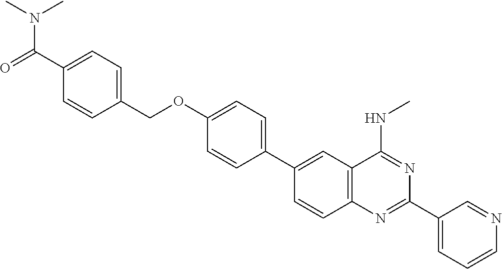 [SMILES: CNc1nc(-c2cccnc2)nc2ccc(-c3ccc(OCc4ccc(C(=O)N(C)C)cc4)cc3)cc12] |
| 1739 | 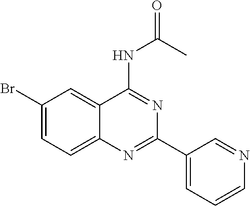 [SMILES: CC(=O)Nc1nc(-c2cccnc2)nc2ccc(Br)cc12] |
| 1740 | 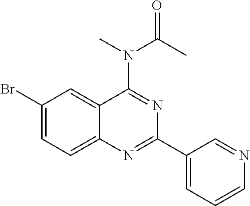 [SMILES: CC(=O)N(C)c1nc(-c2cccnc2)nc2ccc(Br)cc12] |
| 1742 | 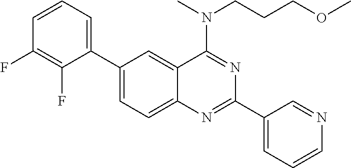 [SMILES: COCCCN(C)c1nc(-c2cccnc2)nc2ccc(-c3cccc(F)c3F)cc12] |
| 1743 | 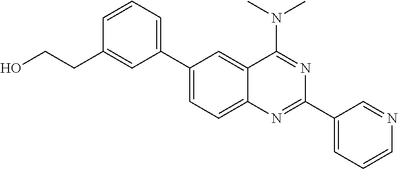 [SMILES: CN(C)c1nc(-c2cccnc2)nc2ccc(-c3cccc(CCO)c3)cc12] |
| 1744 | 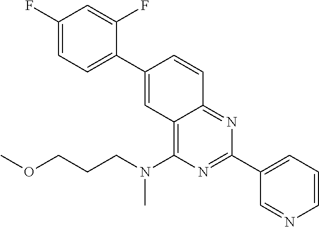 [SMILES: COCCCN(C)c1nc(-c2cccnc2)nc2ccc(-c3ccc(F)cc3F)cc12] |
| 1745 | 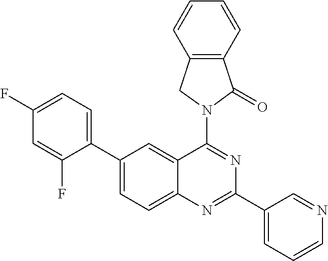 [SMILES: O=C1c2ccccc2CN1c1nc(-c2cccnc2)nc2ccc(-c3ccc(F)cc3F)cc12] |
| 1746 | 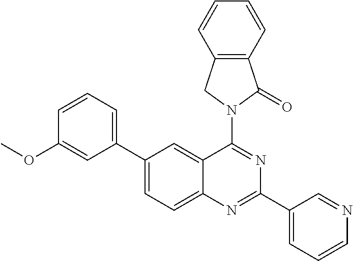 [SMILES: COc1cccc(-c2ccc3nc(-c4cccnc4)nc(N4Cc5ccccc5C4=O)c3c2)c1] |
| 1747 | 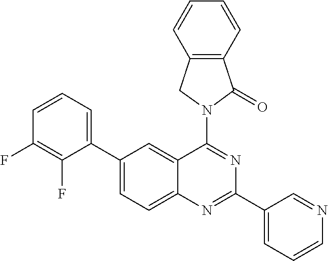 [SMILES: O=C1c2ccccc2CN1c1nc(-c2cccnc2)nc2ccc(-c3cccc(F)c3F)cc12] |
| 1748 | 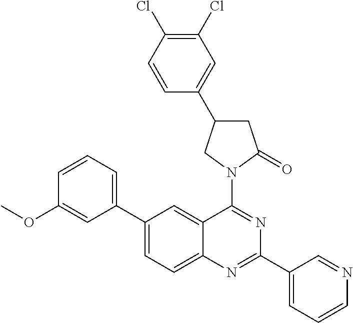 [SMILES: COc1cccc(-c2ccc3nc(-c4cccnc4)nc(N4CC(c5ccc(Cl)c(Cl)c5)CC4=O)c3c2)c1] |
| 1749 | 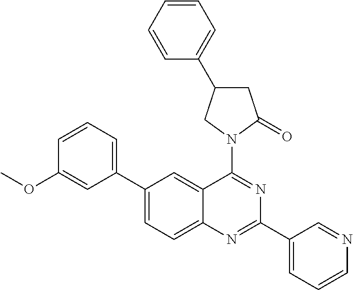 [SMILES: COc1cccc(-c2ccc3nc(-c4cccnc4)nc(N4CC(c5ccccc5)CC4=O)c3c2)c1] |
| 1750 | 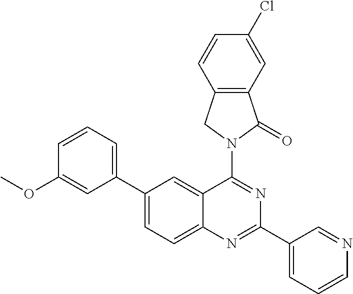 [SMILES: COc1cccc(-c2ccc3nc(-c4cccnc4)nc(N4Cc5ccc(Cl)cc5C4=O)c3c2)c1] |
| 1751 | 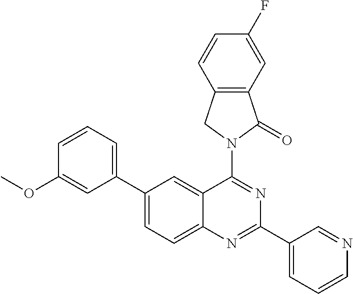 [SMILES: COc1cccc(-c2ccc3nc(-c4cccnc4)nc(N4Cc5ccc(F)cc5C4=O)c3c2)c1] |
| 1752 | 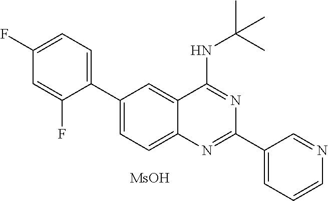 [SMILES: "MsOH" ; CC(C)(C)Nc1nc(-c2cccnc2)nc2ccc(-c3ccc(F)cc3F)cc12] |
| 1753 | 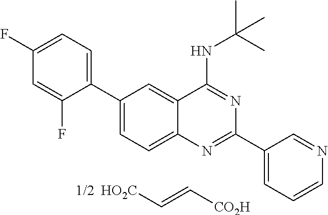 |
| 1754 | 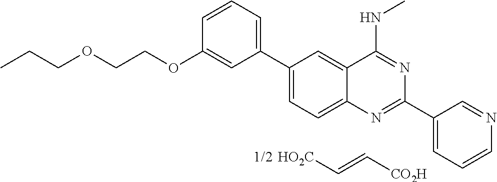 |
| 1755 | 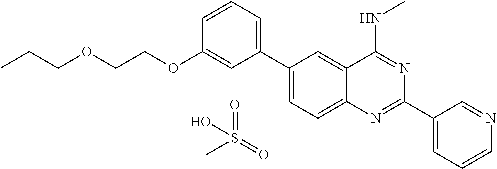 |

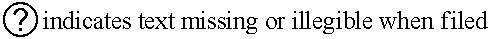

### DETAILED DESCRIPTION

A compound or composition described herein can be used, e.g., in a method of treating schizophrenia or cognitive deficit. Many of the compounds described herein modulate STEP activity and can be used, e.g., to reduce or inhibit STEP activity, e.g., in a subject.

### DEFINITIONS

The term “acyl” refers to an alkylcarbonyl, cycloalkylcarbonyl, arylcarbonyl, heterocyclylcarbonyl, or heteroarylcarbonyl substituent, any of which may be further substituted (e.g., by one or more substituents).

The term “alkenyl” refers to a straight or branched hydrocarbon chain containing 2-12 carbon atoms (unless otherwise noted) and having one or more double bonds. Examples of alkenyl groups include, but are not limited to, allyl, propenyl, 2-butenyl, 3-hexenyl and 3-octenyl groups. One of the double bond carbons may optionally be the point of attachment of the alkenyl substituent.

The term “alkenylene” refers to a divalent alkenyl, e.g. —CH═CH—, —CH₂—CH═CH—, and —CH═CH—CH₂—.

The term “alkynyl” refers to a straight or branched hydrocarbon chain containing 2-12 carbon atoms (unless otherwise noted) and characterized in having one or more triple bonds. Examples of alkynyl groups include, but are not limited to, ethynyl, propargyl, and 3-hexynyl. One of the triple bond carbons may optionally be the point of attachment of the alkynyl substituent.

The term “alkynylene” refers to a divalent alkynyl, e.g. —CH═CH—, —CH₂—CH═CH—, and —CH═CH—CH₂—.

The terms “alkoxyl” or “alkoxy” as used herein refers to an alkyl group, as defined below, having an oxygen radical attached thereto. Representative alkoxy groups include methoxy, ethoxy, propyloxy, tert-butoxy and the like. The term “alkoxyalkyl” refers to an alkyl in which one or more hydrogen atoms are replaced by an alkoxy group.

An “ether” is two hydrocarbons covalently linked by an oxygen.

The term “alkyl” refers to the radical of saturated aliphatic groups, including straight-chain alkyl groups, and branched-chain alkyl groups. In preferred embodiments, a straight chain or branched chain alkyl has 12 or fewer carbon atoms in its backbone (unless otherwise noted) e.g., from 1-12, 1-8, 1-6, or 1-4. Exemplary alkyl moieties include methyl, ethyl, propyl (e.g., n-propyl or isopropyl), butyl (e.g., n-butyl, isobutyl or t-butyl), pentyl (e.g., n-pentyl, isopentyl or pentan-3-yl), hexyl and hepty.

The term “alkylene” refers to a divalent alkyl, e.g., —CH₂—, —CH₂CH₂—, and —CH₂CH₂CH₂—.

The term “amino” refers to —NH₂.

The term “aminoalkyl” refers to an alkyl in which one or more hydrogen atoms are replaced by an amino group.

The terms “alkylamino” and “dialkylamino” refer to —NH(alkyl) and —N(alkyl)₂ radicals respectively.

The term “aralkylamino” or “arylalkylamino” refers to a —NH(aralkyl) radical. The term “alkylaminoalkyl” refers to a (alkyl)NH-alkyl-radical; the term “dialkylaminoalkyl” refers to an (alkyl)₂N-alkyl-radical.

The term “amido” refers to a —NHC(O)— or C(O)NH₂ substituent.

The term “aryl” refers to a 6-carbon monocyclic, 10-carbon bicyclic, or 14-carbon tricyclic aromatic ring system wherein 0, 1, 2, 3, or 4 atoms of each ring may be substituted by a substituent. Examples of aryl moieties include, but are not limited to, phenyl, naphthyl and the like. The term “arylalkyl” or “aralkyl” refers to alkyl substituted with an aryl. Exemplary aralkyls include but are not limited to benzyl, 1-phenylethyl, 2-phenylethyl, 3-phenylpropyl, 9-fluorenyl, benzhydryl, phenethyl, and trityl groups. The term “arylalkenyl” refers to an alkenyl substituted with an aryl. The term “arylalkynyl” refers to an alkynyl substituted with an aryl. Terms such as “arylC₂-C₆alkyl” are to be read as a further limitation on the size of the alkyl group. The term “arylalkoxy” refers to an alkoxy substituted with aryl. The term “arylenyl” refers to a divalent aryl (i.e., —Ar—).

The terms “cycloalkyl” or “cyclyl” as employed herein include saturated and partially unsaturated cyclic hydrocarbon groups having 3 to 12 carbons, preferably 3 to 8 carbons, and more preferably 3 to 6 carbons, wherein the cycloalkyl group may be optionally substituted. Exemplary cyclyl groups include, without limitation, cycloalkyl such as cyclopropyl, cyclobutyl, cyclopentyl, cyclopentenyl, cyclohexyl, cyclohexenyl, cycloheptyl, and cyclooctyl. Cyclyl moieties also include both bridged and fused ring systems. Cyclyl groups also include those that are fused to additional ring systems, which may be saturated or unsaturated. A cyclyl group may thus be a bicyclic group in which one ring is saturated or partially unsaturated and the other is fully unsaturated (e.g., indanyl).

The term “cyclylalkyl” as used herein, refers to an alkyl group substituted with a cyclyl group. Cyclylalkyl includes groups in which more than one hydrogen atom of an alkyl group has been replaced by a cyclyl group.

The term “cycloalkylalkyl” as used herein, refers to an alkyl group substituted with a cycloalkyl group.

The term “halo” or “halogen” refers to any radical of fluorine, chlorine, bromine or iodine.

The term “haloalkyl” refers to an alkyl group that may have any number of hydrogens available on the group replaced with a halogen atom. Representative haloalkyl groups include but are not limited to: —CH₂Cl, —CH₂ClCF₃, —CHBr₂, —CF₃, —CH₂F, —CHF₂, and —CH₂CF₃. The term “fluoroalkyl” refers to an alkyl group that may have any number of hydrogens available on the group replaced with a fluorine atom. Representative fluoroalkyl groups include but are not limited to: —CH₂F, —CH₂FCF₃, —CHF₂ and —CF₃. The term “haloalkoxy” refers to an alkoxy group that may have any number of hydrogen atoms available on the alkyl group replaced with a halogen atom. Representative haloalkoxy groups include but are not limited to: —OCH₂Cl, —OCH₂ClCF₃, —OCHBr₂, —OCHF₂ or —OCF₃. The term “fluoroalkoxy” refers to an alkoxy group that may have any number of hydrogens available on the group replaced with a fluorine atom. Representative fluoroalkoxy groups include but are not limited to: —OCH₂F, —OCH₂FCF₃, —OCHF₂ or —OCF₃.

The term “heteroatom” as used herein means an atom of any element other than carbon or hydrogen. Preferred heteroatoms are nitrogen, oxygen, sulfur, phosphorus and silicon. A heteroatom may be present in any oxidation state (e.g., any oxidized form of nitrogen, sulfur, phosphorus or silicon) and any charged state (e.g., the quaternized form of any basic nitrogen), and includes a substitutable nitrogen of a heterocyclic ring, for example N (as in 3,4-dihydro-2H-pyrrolyl), NH (as in pyrrolidinyl) or NR⁺ (as in N-substituted pyrrolidinyl).

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The term “heteroarylalkyl” or the term “heteroaralkyl” refers to an alkyl substituted with a heteroaryl. The term “heteroarylalkenyl” refers to an alkenyl substituted with a heteroaryl. The term “heteroarylalkynyl” refers to an alkynyl substituted with a heteroaryl. The term “heteroarylalkoxy” refers to an alkoxy substituted with heteroaryl.

The term “heteroaryl” refers to a group having 5 to 14 ring atoms, preferably 5, 6, 9, or 10 ring atoms; having 6, 10, or 14 π electrons shared in a cyclic array; and having, in addition to carbon atoms, from one to five heteroatoms. A heteroaryl group may be mono-, bi-, tri-, or polycyclic, preferably mono-, bi-, or tricyclic, more preferably mono- or bicyclic. When a heteroaryl is substituted by a hydroxy group, it also includes its corresponding tautomer. The term “heteroaryl,” as used herein, also includes groups in which a heteroaromatic ring is fused to one or more aryl rings. Nonlimiting examples of heteroaryl groups include thiophenyl or thienyl, furyl or furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. The term “heteroaryl” may be used interchangeably with the terms “heteroaryl ring”, “heteroaryl group,” or “heteroaromatic,” any of which terms include rings that are optionally substituted. A ring nitrogen atom of a heteroaryl may be oxidized to form the corresponding N-oxide compound. A nonlimiting example of such a heteroaryl having an oxidized ring nitrogen atom is N-oxopyridyl.

The term “heteroarylalkyl” or “heteroaralkyl” refers to an alkyl group substituted by a heteroaryl. Heteroaralkyl includes groups in which more than one hydrogen atom has been replaced by a heteroaryl group.

As used herein, the terms “heterocycle,” “heterocyclyl” and “heterocyclic ring” are used interchangeably and refer to a stable 3- to 8-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2/y-pyrrolyl), NH (as in pyrrolidinyl), or NR⁺ (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydropyranyl, tetrahydrothienyl, piperidinyl, decahydroquinolinyl, oxazolidinyl, piperazinyl, dioxanyl, dioxolanyl, diazepinyl, oxazepinyl, thiazepinyl, morpholinyl, and thiomorpholinyl. A heterocyclyl group may be mono-, bi-, tri-, or polycyclic, preferably mono-, bi-, or tricyclic, more preferably mono- or bicyclic. Additionally, a heterocyclic ring also includes groups in which the heterocyclyl ring is fused to one or more aryl, heteroaryl or cyclyl rings. A ring nitrogen atom of a heterocyclic ring also may be oxidized to form the corresponding N-hydroxy compound.

The term “heterocyclylalkyl” refers to an alkyl group substituted by a heterocyclyl. Heterocyclylalkyl includes groups in which one or more hydrogen atom has been replaced by a heterocyclyl group.

The terms “hetaralkyl” and “heteroaralkyl”, as used herein, refers to an alkyl group substituted with a heteroaryl group. Exemplary heteroaralkyl groups include but are not limited to methylpyridyl or methylpyrimidyl.

The term “heterocyclyl” or “heterocyclylalkyl” refers to a nonaromatic 5-8 membered monocyclic, 5-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and include both bridged and fused ring systems. The term “heterocyclylalkyl” refers to an alkyl substituted with a heterocyclyl.

The term “heterocyclylalkyl”, as used herein, refers to an alkyl group substituted with a heterocycle group.

The term “heteroalkyl,” as used herein, refers to a saturate or unsaturated, straight or branched chain aliphatic group, wherein one or more of the carbon atoms in the chain are independently replaced by a heteroatom. Exemplary hetero atoms include O, S, and N.

In the case of aralkyl, heteroaralkyl, cyclylalkyl, heterocyclylalkyl etc., groups described as optionally substituted, it is intended that either or both aryl, heteroaryl, cyclyl, heterocyclyl and alkyl moieties may be independently optionally substituted or unsubstituted.

The term “hydroxyalkyl” refers to an alkyl in which one or more hydrogen atoms are replaced by a hydroxy group.

The term “oxo” refers to an oxygen atom (═O), which forms a carbonyl when attached to carbon, an N-oxide when attached to nitrogen, and a sulfoxide or sulfone when attached to sulfur.

The term “thioalkyl” as used herein refers to an —S(alkyl) group, where the point of attachment is through the sulfur atom and the alkyl group is as defined above.

The term “thiono” or “thioxo” refers to a sulfur atom (═S), which forms a thioketone when attached to carbon.

The term “substituted” refers to the fact that moieties have one or more substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms.

The term “substituent” refers to a group substituted for a hydrogen atom on a moiety described herein. Any atom on any substituent can be substituted. Substituents can include any substituents described herein. Exemplary substituents include, without limitation, alkyl (e.g., C1, C2, C3, C4, C5, C6, C7, C8, C9, C10, C11, C12 straight or branched chain alkyl), cycloalkyl, haloalkyl (e.g., perfluoroalkyl such as CF₃), aryl, heteroaryl, aralkyl, heteroaralkyl, heterocyclyl, alkenyl, alkynyl, cycloalkenyl, heterocycloalkenyl, alkoxy, haloalkoxy (e.g., perfluoroalkoxy such as OCF₃), halo, hydroxy, carboxy, carboxylate, cyano, nitro, amino, alkylamino, SO₃H, sulfate, phosphate, methylenedioxy (—O—CH₂—O— wherein oxygens are attached to vicinal atoms), ethylenedioxy, oxo, thioxo (e.g., C═S), imino (alkyl, aryl, aralkyl), S(O)ₙalkyl (where n is 0-2), S(O)ₙaryl (where n is 0-2), S(O)ₙheteroaryl (where n is 0-2), S(O)ₙheterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di-, alkyl, aralkyl, heteroaralkyl, and combinations thereof). In one aspect, the substituents on a group are independently any one single, or any subset of the aforementioned substituents. In another aspect, a substituent may itself be substituted with any one of the above substituents.

As used herein, the phrase “optionally substituted” is used interchangeably with the phrase “substituted or unsubstituted.” In general, the term “substituted”, whether preceded by the term “optionally” or not, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which hydrogen atom can be replaced with the radical of a suitable substituent. Unless otherwise indicated, an “optionally substituted” group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

As used herein, the term “optionally substituted” means substituted or unsubstituted.

As used herein, the term “partially unsaturated” refers to a moiety that includes at least one double or triple bond between atoms. The term “partially unsaturated” encompasses rings, e.g., having one or more sites of unsaturation, but that are not completely unsaturated so as to be aryl or heteroaryl.

The term “chiral” refers to molecules which have the property of non-superimposability of the mirror image partner, while the term “achiral” refers to molecules which are superimposable on their mirror image partner. With respect to the nomenclature of a chiral center, terms “R” and “S” configuration are as defined by the IUPAC Recommendations. The term “enantiomers” refers to two stereoisomers of a compound which are non-superimposable mirror images of one another. An equimolar mixture of two enantiomers is called a “racemic mixture” or a “racemate.” The term “isomers” or “stereoisomers” refers to compounds which have identical chemical constitution, but differ with regard to the arrangement of the atoms or groups in space. For example, isomers include cis- and trans-isomers, E- and Z-isomers, R- and S-enantiomers, diastereomers, (D)-isomers, (L)-isomers, racemic mixtures thereof, and other mixtures thereof. The term “diastereomers” refers to stereoisomers with two or more centers of dissymmetry and whose molecules are not mirror images of one another.

The term “administration” or “administering” includes routes of introducing the compounds, or a composition thereof, of the invention to a subject to perform their intended function. Examples of routes of administration that may be used include injection (subcutaneous, intravenous, parenterally, intraperitoneally, intrathecal), oral, inhalation, rectal and transdermal. The pharmaceutical compositions may be given by forms suitable for each administration route. For example, these compositions are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, a compound described herein can be coated with or disposed in a selected material to protect it from natural conditions which may detrimentally affect its ability to perform its intended function. A compound or composition described herein can be administered alone, or in conjunction with either another agent as described above or with a pharmaceutically-acceptable carrier, or both. A compound or composition described herein can be administered prior to the administration of the other agent, simultaneously with the agent, or after the administration of the agent. Furthermore, a compound described herein can also be administered in a pro-drug form which is converted into its active metabolite, or more active metabolite in vivo.

The language “biological activities” of a compound described herein includes all activities elicited by a compound described herein in a responsive subject or cell. It includes genomic and non-genomic activities elicited by these compounds.

The terms “inhibit” and “inhibitor” as used herein means an agent that measurably slows or stops the production of STriatal-Enriched tyrosine Phosphatase (STEP), or decreases or inactivates STEP, or interferes with STEP-mediated biological pathways. Inhibitors of STEP include compounds of the invention, e.g., compounds of Formulas (I)-(IX′). A compound can be evaluated to determine if it is an inhibitor by measuring either directly or indirectly the activity of STEP in the presence of the compound suspected to inhibit STEP. Exemplary methods of measure STEP inhibition are described in the EXAMPLES herein.

An “effective amount” or “an amount effective” refers to an amount of the compound or composition which is effective, upon single or multiple dose administrations to a subject and for periods of time necessary, in treating a cell, or curing, alleviating, relieving or improving a symptom of a disorder, e.g., a disorder described herein. An effective amount of a compound described herein may vary according to factors such as the disease state, age, and weight of the subject, and the ability of a compound described herein to elicit a desired response in the subject. Dosage regimens may be adjusted to provide the optimum therapeutic response. An effective amount is also one in which any toxic or detrimental effects (e.g., side effects) of a compound described herein are outweighed by the therapeutically beneficial effects. The term “effective amount” includes an amount effective, at dosages and for periods of time necessary, to achieve the desired result, e.g., modulate or regulate protein tyrosine phosphatases, e.g., STEP, in a subject and/or treat a disorder described herein such as a protein tyrosine phosphatase related disorder. Exemplary disorders include those related to cognition, learning and memory, neurogenesis. An effective amount may also affect neuronal plasticity, pain perception, mood and anxiety, and neuroendocrine regulation.

An effective amount of a compound described herein may vary according to factors such as the disease state, age, and weight of the subject, and the ability of a compound described herein to elicit a desired response in the subject. Dosage regimens may be adjusted to provide the optimum therapeutic response. An effective amount is also one in which any toxic or detrimental effects (e.g., side effects) of a compound described herein are outweighed by the therapeutically beneficial effects.

A therapeutically effective amount of a compound described herein (i.e., an effective dosage) may range from about 0.001 to 50 mg/kg body weight, preferably about 0.01 to 40 mg/kg body weight, more preferably about 0.1 to 35 mg/kg body weight, still more preferably about 1 to 30 mg/kg, and even more preferably about 10 to 30 mg/kg. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound described herein can include a single treatment or, preferably, can include a series of treatments. In one example, a subject is treated with a compound described herein in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a compound described herein used for treatment may increase or decrease over the course of a particular treatment.

As used herein, an amount of a compound effective to prevent a disorder, or “a prophylactically effective amount” of the compound refers to an amount effective, upon single- or multiple-dose administration to the subject, in preventing or delaying the occurrence of the onset or recurrence of a disorder or a symptom of the disorder.

The language “improved biological properties” refers to any activity inherent in a compound described herein that enhances its effectiveness in vivo. In a preferred embodiment, this term refers to any qualitative or quantitative improved therapeutic property of a compound described herein, such as reduced off-target effects.

The term “modulate” refers to an increase or decrease, e.g., in the activity of an enzyme in response to exposure to a compound or composition described herein, e.g., the activation or inhibition of STEP, in at least a sub-population of cells in a subject such that a desired end result is achieved (e.g., a therapeutic result). In some embodiments, a compound as described herein inhibits a target described herein, e.g., STEP. In some embodiments, a compound as described herein is activates a target described herein, e.g., STEP.

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dog, cat, cow, pig, etc.

As used herein, the term “treat” or “treating” is defined as applying or administering a compound or composition, alone or in combination with a second compound or composition, to a subject, e.g., a patient, or applying or administering the compound or composition to an isolated tissue or cell, e.g., cell line, from a subject, e.g., a patient, who has a disorder (e.g., a disorder as described herein), a symptom of a disorder, or a predisposition toward a disorder, with the purpose to cure, heal, alleviate, relieve, alter, remedy, ameliorate, improve or affect the disorder, one or more symptoms of the disorder or the predisposition toward the disorder (e.g., to prevent at least one symptom of the disorder or to delay onset of at least one symptom of the disorder).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The term “prodrug” or “pro-drug” includes compounds with moieties that can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The language “a prophylactically effective amount” of a compound refers to an amount of a compound described herein any formula herein or otherwise described herein which is effective, upon single or multiple dose administration to the patient, in preventing or treating a disease or condition.

The language “reduced off-target effects” is intended to include a reduction in any undesired side effect elicited by a compound described herein when administered in vivo. In some embodiments, a compound described herein has little to no cardio and/or pulmonary toxicity (e.g., when administered to a subject). In some embodiments, a compound described herein has little to no hallucinogenic activity (e.g., when administered to a subject).

The term “selective” means a greater activity against a first target. In some embodiments a compound has a selectivity of at least 1.25-fold, at least 1.5 fold, at least 2-fold, at least 3-fold, at least 4-fold, at least 5-fold, at least 6-fold, at least 10-fold or at least 100-fold greater towards a first target relative to a second target. In some embodiments, a compound described herein, e.g., a compound of Formulas (I)-(IX′) is selective toward STEP relative to one or more other protein tyrosine phosphatases.

The term “subject” includes organisms which are capable of suffering from a serotonin-receptor-related disorder or who could otherwise benefit from the administration of a compound described herein of the invention, such as human and non-human animals. Preferred humans include human patients suffering from or prone to suffering from a serotonin-related disorder or associated state, as described herein. The term “non-human animals” of the invention includes all vertebrates, e.g., mammals, e.g., rodents, e.g., mice, and non-mammals, such as non-human primates, e.g., sheep, dog, cow, chickens, amphibians, reptiles, etc.

The phrases “systemic administration,” “administered systemically”, “peripheral administration” and “administered peripherally” as used herein mean the administration of a compound described herein(s), drug or other material, such that it enters the patient's system and, thus, is subject to metabolism and other like processes, for example, subcutaneous administration.

Compounds

The compounds described herein can be used for a variety of purposes, e.g., therapeutic purposes. Many of the compounds modulate STEP activity and can be used, for example, to inhibit STEP, e.g., in a subject.

Exemplary compounds include a compound of formula (I):

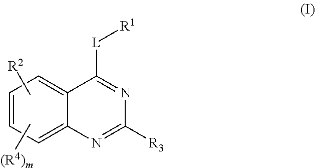

wherein L, R¹, R², R³, R⁴, and m are as defined above in the section relating to formula (I).

Exemplary compounds include a compound of formula (II):

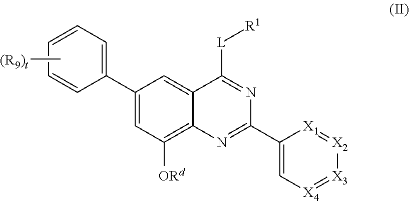

wherein L, R¹, R⁹, R^{d}, X₁, X₂, X₃, X₄, and t are as defined above in the section relating to formula (II).

Exemplary compounds include a compound of formula (III):

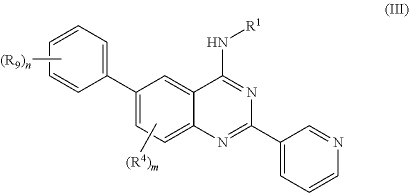

wherein L, R¹, R⁴, R⁹, m, and n are as defined above in the section relating to formula (III).

Exemplary compounds include a compound of formula (IV):

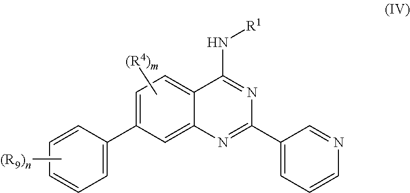

wherein R¹, R⁴, R⁹, m, and n are as defined above in the section relating to formula (IV).

Exemplary compounds include a compound of formula (V):

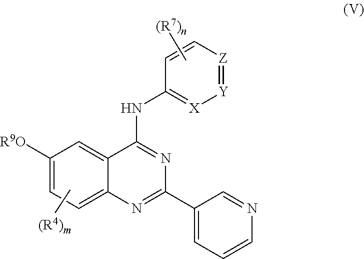

wherein R⁴, R⁷, R⁹, X, Y, Z, m, and n are as defined above in the section relating to formula (V).

Exemplary compounds include a compound of formula (VI):

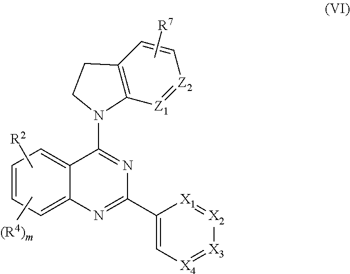

wherein R², R⁴, R⁷, X₁, X₂, X₃, X₄, Z₁, Z₂, and m are as defined above in the section relating to formula (VI).

Exemplary compounds include a compound of formula (VII):

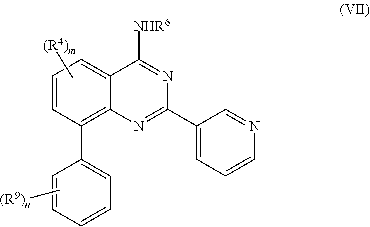

wherein R⁴, R⁶, R⁹, m, and n are as defined above in the section relating to formula (VII).

Exemplary compounds include a compound of formula (VIII):

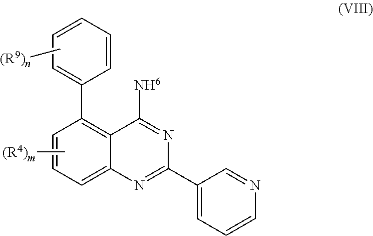

wherein R⁴, R⁶, R⁹, m, and n are as defined above in the section relating to formula (VIII).

Exemplary compounds include a compound of formula (IX) or (IX′):

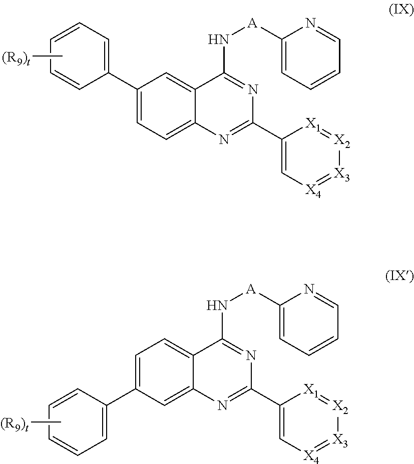

wherein A, R⁹, X₁, X₂, X₃, X₄, and t are as defined above in the section relating to formula (I).

The present invention includes compounds that differ only in the presence of one or more isotopically enriched atoms. For example, compounds having the present structures except for the replacement of hydrogen by deuterium or tritium, the replacement of a carbon by a ¹³C- or ¹⁴C-enriched carbon, or the replacement of a fluorine by a ¹⁹F-enriched fluorine are within the scope of this invention. Such compounds are useful, for example, as analytical tools or probes in biological assays, or as bioactive agents.

In the compounds of the present invention, any atom not specifically designated as a particular isotope is meant to represent any stable isotope of that atom unless otherwise stated (e.g., hydrogen, ²H or deuterium and ³H or tritium). The formulas described herein may or may not indicate whether atoms at certain positions are isotopically enriched. When a structural formula is silent with respect to whether a particular position is isotopically enriched, it is to be understood that the isotopes at that particular position are present in natural abundance or, that the particular position is isotopically enriched with one or more naturally occurring stable isotopes. For example, the formula —CH₂— represents the following possible structures: —CH₂—, —CHD- or —CD₂-.

The variable “D” is defined as deuterium.

The terms “compound” or “compounds,” when referring to a compound of this invention or a compound described herein, refers to a collection of molecules having an identical chemical structure, except that there may be isotopic variation among the constituent atoms of the molecules. Thus, it will be clear to those of skill in the art that a compound represented by a particular chemical structure containing indicated hydrogen atoms will contain lesser amounts of isotopologues having deuterium atoms at one or more of the designated hydrogen positions in that structure. Alternatively, a compound represented by a particular chemical structure containing indicated deuterium atoms will contain lesser amounts of isotopologues having hydrogen atoms at one or more of the designated deuterium positions in that structure. The relative amount of such isotopologues in a compound of this invention will depend on a number of factors including isotopic purity of deuterated reagents used to make the compound and the efficiency of incorporation of deuterium in the various synthetic steps used to prepare the compound. The relative amount of such isotopologues in total will be less than 55% of the compound. In other embodiments, the relative amount of such isotopologues in total will be less than 50%, less than 45%, less than 40%, less than 35%, less than 35%, less than 15%, less than 10%, less than 5%, less than 1% or less than 0.5% of the compound.

The term “isotopologue” refers to a species that differs from a specific compound of this invention only in the isotopic composition thereof. Isotopologues can differ in the level of isotopic enrichment at one or more positions and/or in the position(s) of isotopic enrichment.

The compounds of this invention may contain one or more asymmetric centers and thus occur as racemates and racemic mixtures, single enantiomers, individual diastereomers and diastereomeric mixtures. Described herein are enantiomerically enriched compounds (e.g., a compound resolved to an enantiomeric excess of 60%, 70%, 80%, 85%, 90%, 95%, 99% or greater). All such isomeric forms of these compounds are expressly included in the present invention. The compounds of this invention may also contain linkages (e.g., carbon-carbon bonds) or substituents that can restrict bond rotation, e.g. restriction resulting from the presence of a ring or double bond. Accordingly, all cis/trans and E/Z isomers are expressly included in the present invention. The compounds of this invention may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts. For example a compound can be resolved to an enantiomeric excess (e.g., 60%, 70%, 80%, 85%, 90%, 95%, 99% or greater) via formation of diasteromeric salts, e.g. with a chiral base, e.g., (+) or (−) α-methylbenzylamine, or via high performance liquid chromatography using a chiral column. In some embodiments a product is purified directly on a chiral column to provide enantiomerically enriched compound.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic administration to a subject).

Compounds of formulas (I)-(IX′) are described herein, for example as provided in the summary above. Exemplary compounds are shown in Tables X-XX in the Examples section.

Synthetic Methods

A compound described herein may be prepared via a variety of synthetic methods. General routes for the synthesis of compounds disclosed herein and representative syntheses of selected compounds disclosed herein are shown in the Examples section.

As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Additionally, the compounds disclosed herein can be prepared on a solid support. The term “solid support” refers a material to which a compound is attached to facilitate identification, isolation, purification, or chemical reaction selectivity of the compound. Such materials are known in the art and include, for example, beads, pellets, disks, fibers, gels, or particles such as cellulose beads, pore-glass beads, silica gels, polystyrene beads optionally cross-linked with divinylbenzene and optionally grafted with polyethylene glycol, poly-acrylamide beads, latex beads, dimethylacrylamide beads optionally cross-linked with N,N′-bis-acryloyl ethylene diamine, glass particles coated with hydrophobic polymer, and material having a rigid or semi-rigid surface. The solid supports optionally have functional groups such as amino, hydroxy, carboxy, or halo groups, (see, Obrecht, D. and Villalgrodo, J. M., Solid-Supported Combinatorial and Parallel Synthesis of Small-Molecular-Weight Compound Libraries, Pergamon-Elsevier Science Limited (1998)), and include those useful in techniques such as the “split and pool” or “parallel” synthesis techniques, solid-phase and solution-phase techniques, and encoding techniques (see, for example, Czarnik, A. W., Curr. Opin. Chem. Bio., (1997) 1, 60).

A compound described herein may be modified by appending appropriate functionalities to enhance selective biological properties. Such modifications are known in the art and include those which increase biological penetration into a given biological compartment (e.g., brain, blood, lymphatic system, central nervous system), increase oral availability, increase solubility to allow administration by injection, alter metabolism and alter rate of excretion.

Included herein are pharmaceutically acceptable derivatives or prodrugs of the compounds described herein. A “pharmaceutically acceptable derivative or prodrug” means any pharmaceutically acceptable salt, ester, salt of an ester, or other derivative of a compound of this invention (for example an imidate ester of an amide), which, upon administration to a recipient, is capable of providing (directly or indirectly) a compound described herein. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. In an exemplary embodiment, the prodrug is a derivative including a group that enhances aqueous solubility or active transport through the gut membrane is appended to the structure of formulae described herein. In another exemplary embodiment, the prodrug is suitable for treatment or prevention of those diseases and conditions that require the drug molecule to cross the blood brain barrier. In a preferred embodiment, the prodrug enters the brain, where it is converted into the active form of the drug molecule.

Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization.

### Evaluating Compounds

A variety of methods can be used to evaluate a compound for ability to modulate STEP activity. Evaluation methods include in vitro assays (e.g., enzyme-based assays), in vitro cell-based signaling assays, and in vivo methods (e.g., testing in animal models). The evaluation methods can evaluate binding activity, phosphatase activity, or an activity downstream of STEP, such as the activity of ERK.

For example, a compound described herein may be evaluated using a fluorescence-based phosphatase assay. A phosphate-containing reagent may be used in the assay which, upon dephosphorylation by a phosphatase, generates a fluorescent product that may be detected using a fluorometer or fluorescence plate reader. Data may be expressed as percentage (%) inhibition of enzyme activity. For compounds showing enzymatic activation, data may be represented as percentage of inhibition but with negative values.

Compositions and Routes of Administration

The invention also provides a pharmaceutical composition, comprising an effective amount of a compound described herein (e.g., a compound capable of treating or preventing a condition as described herein, e.g., a compound of any formula herein or otherwise described herein) and a pharmaceutically acceptable carrier.

The compositions delineated herein include the compounds delineated herein (e.g., a compound described herein), as well as additional therapeutic agents if present, in amounts effective for achieving a modulation of disease or disease symptoms, including those described herein.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

When the compositions of this invention comprise a combination of a compound of the formulae described herein and one or more additional therapeutic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The compounds described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

Methods of Treatment

The compounds and compositions described herein can be administered to cells in culture, e.g. in vitro or ex vivo, or to a subject, e.g., in vivo, to treat, prevent, and/or diagnose a variety of disorders, including those described herein below.

The compounds and compositions described herein can be administered to a subject, for example using a method described herein, who is suffering from a disorder described herein, e.g., a disorder that would benefit from the modulation of STEP (e.g., activating or inhibiting STEP). The compounds and compositions described herein can be administered to a subject, for example using a method described herein, who is at risk for a disorder described herein, e.g., a disorder that would benefit from the modulation of STEP (e.g., activating or inhibiting STEP).

Inhibitors of STEP may increase phosphorylation of an NMDA-R. Thus, in some embodiments, a compound described herein, e.g., a compound that inhibits STEP, may be useful for treating a disorder in which increasing phosphorylation of an NMDA-R would be beneficial.

Inhibitors of STEP may activate an ERK1 or ERK2 kinase, for example, in the CNS. Thus, in some embodiments, a compound described herein, e.g., a compound that inhibits STEP, may be useful for treating a disorder in which activate an ERK1 or ERK2 kinase would be beneficial.

Compounds described herein may be useful in treating a variety of disorders, including disorders of the CNS. Exemplary disorders include schizophrenia, schizoaffective disorders, major depression, bipolar disorder, cognitive deficit, mild cognitive impairment (MCI), Alzheimer's disease (AD), attention-deficit/hyperactivity disorder (ADHD), dementia, generalized anxiety disorders, panic disorders, obsessive-compulsive disorders, phobias, post-traumatic stress syndrome, anorexia nervosa, drug addiction, ischemic stroke, head trauma or brain injury, Huntington's disease, Parkinson's disease, spinocerebellar degeneration, motor neuron diseases, epilepsy, neuropathic pain, chronic pain, neuropathies, autism and autistic disorders.

Compounds described herein may be useful for treating or preventing central nervous system disorders selected from the group consisting of schizophrenia; refractory, intractable or chronic schizophrenia; emotional disturbance; psychotic disorder; mood disorder; bipolar I type disorder; bipolar II type disorder; depression; endogenous depression; major depression; melancholy and refractory depression; dysthymic disorder; cyclothymic disorder; panic attack; panic disorder; agoraphobia; social phobia; obsessive-compulsive disorder; post-traumatic stress disorder; generalized anxiety disorder; acute stress disorder; hysteria; somatization disorder; conversion disorder; pain disorder; hypochondriasis; factitious disorder; dissociative disorder; sexual dysfunction; sexual desire disorder; sexual arousal disorder; erectile dysfunction; anorexia nervosa; bulimia nervosa; sleep disorder; adjustment disorder; alcohol abuse; alcohol intoxication; drug addiction; stimulant intoxication; narcotism; anhedonia; iatrogenic anhedonia; anhedonia of a psychic or mental cause; anhedonia associated with depression; anhedonia associated with schizophrenia; delirium; cognitive impairment; cognitive impairment associated with Alzheimer's disease, Parkinson's disease and other neurodegenerative diseases; cognitive impairment caused by Alzheimer's disease; Parkinson's disease and associated neurodegenerative diseases; cognitive impairment of schizophrenia; cognitive impairment caused by refractory, intractable or chronic schizophrenia; vomiting; motion sickness; obesity; migraine; pain (ache); mental retardation; autism disorder (autism); Tourette's disorder; tic disorder; attention-deficit/hyperactivity disorder; conduct disorder; and Down's syndrome.

Compounds described herein may be useful for treating or preventing disorders selected from schizophrenia, schizoaffective disorder, bipolar disorder, manic-depressive disorder, psychosis, mood and anxiety disorders, mania, drug or substance addiction, cognition disorders, learning disabilities, learning and memory disorders, aging and neurologic disorders associated with or linked with cognitive impairments; mild cognitive impairments (MCI), Alzheimer's disease, Alzheimer-related cognition disorders, Huntington's disease, Parkinson's disease, CADASIL syndrome (cerebral autosomal dominant arteriopathy with subcortical infarcts and leukoencephalopathy), amnesia, Wernicke-Korsakoff syndrome, Korsakoff syndrome, mild traumatic head injury (MBTI), traumatic head injury (TBI), fragile X syndrome, stroke, attention-deficit and hyperactivity disorder (ADHD), obsessive compulsive disorder (OCD), post-traumatic stress disorder (PTSD), loss of concentration, autism, cerebral palsy, encephalopathy, and narcolepsy. The disorder may affect learning and memory, neurogenesis, neuronal plasticity, pain perception, mood and anxiety, or neuroendocrine regulation. The disorder may be a cognitive deficit disorder. The disorder may involve pain perception or neuroendocrine regulation.

Compound described herein also shows low toxicity, and is safely administered to mammals (e.g., rat, mouse, guinea pig, rabbit, sheep, horse, pig, cow, monkey, human).

Schizophrenia

In some embodiments, a compound or composition described herein can be used in the treatment of schizophrenia. Schizophrenia is a psychiatric diagnosis that describes a mental disorder characterized by abnormalities in the perception or expression of reality. Distortions in perception may affect all five senses, including sight, hearing, taste, smell and touch, but most commonly manifests as auditory hallucinations, paranoid or bizarre delusions, or disorganized speech and thinking with significant social or occupational dysfunction. Onset of symptoms typically occurs in young adulthood, with approximately 0.4-0.6% of the population affected. Diagnosis is based on the patient's self-reported experiences and observed behavior.

The disorder is thought to mainly affect cognition, but it also usually contributes to chronic problems with behavior and emotion. People with schizophrenia are likely to have additional (comorbid) conditions, including major depression and anxiety disorders. Social problems, such as long-term unemployment, poverty and homelessness, are common. Furthermore, the average life expectancy of people with the disorder is 10 to 12 years less than those without, due to increased physical health problems and a higher suicide rate.

The Diagnostic and Statistical Manual of Mental Disorders (DSM) contains five sub-classifications of schizophrenia. These include Paranoid type (where delusions and hallucinations are present but thought disorder, disorganized behavior, and affective flattening are absent); Disorganized type (also known as hebephrenic schizophrenia, where thought disorder and flat affect are present together); Catatonic type (the subject may be almost immobile or exhibit agitated, purposeless movement; symptoms can include catatonic stupor and waxy flexibility); Undifferentiated type (psychotic symptoms are present but the criteria for paranoid, disorganized, or catatonic types have not been met); and Residual type (where positive symptoms are present at a low intensity only).

The International Statistical Classification of Diseases and Related Health Problems (10th Revision) defines two additional subtypes. These include Post-schizophrenic depression (a depressive episode arising in the aftermath of a schizophrenic illness where some low-level schizophrenic symptoms may still be present); and Simple schizophrenia (insidious and progressive development of prominent negative symptoms with no history of psychotic episodes.)

An agent for the treatment of schizophrenia may improve so-called positive symptoms in the acute period of schizophrenia such as hallucinations, delusions, excitations and the like. An agent for treating schizophrenia may also improve so-called negative symptoms that are observed in the chronic period of schizophrenia such as apathy, emotional depression, hyposychosis and the like.

Schizoaffective Disorder

Schizoaffective disorder is a psychiatric diagnosis that describes a mental disorder characterized by recurring episodes of elevated or depressed mood, or simultaneously elevated and depressed mood that alternate or occur together with distortions in perception. The perceptual distortion component of the disorder, called psychosis, may affect all five senses, including sight, hearing, taste, smell and touch, but most commonly manifest as auditory hallucinations, paranoid or bizarre delusions, or disorganized speech and thinking with significant social and occupational dysfunction. The elevated, depressed or simultaneously elevated and depressed mood episode components of the disorder, called mood disorder, are broadly recognized as depressive and bipolar types of the illness; the division is based on whether the individual has ever had a manic, hypomanic or mixed episode. Onset of symptoms usually begins in early adulthood and is rarely diagnosed in childhood (prior to age 13). The lifetime prevalence of the disorder is uncertain (due to studies using varying diagnostic criteria), although it is generally agreed to be less than 1 percent, and possibly in the range of 0.5 to 0.8 percent. Diagnosis is based on the patient's self-reported experiences and observed behavior. No laboratory test for schizoaffective disorder currently exists. As a group, people with schizoaffective disorder have a more favorable prognosis than people with schizophrenia, but a worse prognosis than those with mood disorders.

The disorder is thought to mainly affect cognition and emotion, but it also usually contributes to ongoing problems with behavior and motivation. People with schizoaffective disorder are likely to have additional (comorbid) conditions, including anxiety disorders and substance abuse. Social problems, such as long-term unemployment, poverty and homelessness, are common. Furthermore, the average life expectancy of people with the disorder is shorter than those without the disorder, due to increased physical health problems and a higher suicide rate.

Cognitive Deficit

Treatment using a compound or composition described herein may improve a cognitive deficit associated with a cognition-related disorder. Cognitive deficit is an inclusive term to describe any characteristic that acts as a barrier to cognitive performance. The term may describe deficits in global intellectual performance, such as mental retardation, it may describe specific deficits in cognitive abilities (learning disorders, dyslexia), or it may describe drug-induced cognitive/memory impairment, such as that seen with alcohol and the benzodiazepines. Cognitive deficits may be congenital or caused by environmental factors such as brain injuries, neurological disorders, or mental illness.

Exemplary cognition-related disorders (e.g., cognitive dysfunction) include, without limitation, mild cognitive impairment (MCI), dementia, delirium, amnestic disorder, Alzheimer's disease, Parkinson's disease and Huntington's disease; memory disorders including memory deficits associated with depression, senile dementia, dementia of Alzheimer's disease; cognitive deficits or cognitive dysfunction associated with neurological conditions including, for example, Parkinson's disease (PD), Huntington's disease (HD), Alzheimer's disease, depression, schizophrenia and other psychotic disorders such as paranoia and manic-depressive illness; cognitive dysfunction in schizophrenia; disorders of attention and learning such as attention deficit disorders (e.g., attention deficit hyperactivity disorder (ADHD)) and dyslexia; cognitive dysfunction associated with developmental disorders such as Down's syndrome and Fragile X syndrome; loss of executive function; loss of learned information; vascular dementia; schizophrenia; cognitive decline; a neurodegenerative disorder; and other dementias, for example, dementia due to HIV disease, head trauma, Parkinson's disease, Huntington's disease, Pick's disease, Creutzfeldt-Jakob disease, or due to multiple etiologies. Cognition-related disorders also include, without limitation, cognitive dysfunction associated with MCI and dementias such as Lewy Body, vascular, and post stroke dementias. Cognitive dysfunction associated with surgical procedures, traumatic brain injury or stroke may also be treated in accordance with the embodiments described herein.

Major Depression

Major depression (also known as clinical depression, major depressive disorder, unipolar depression, or unipolar disorder) is a mental disorder characterized by a pervasive low mood, low self-esteem, and loss of interest or pleasure in normally enjoyable activities. Types of Major depressive disorder include, e.g., Atypical depression, Melancholic depression, Psychotic depression, Catatonic depression, Postpartum depression, and Seasonal affective disorder.

Bipolar Disorder

Bipolar disorder, also known as manic depressive disorder, manic depressive psychosis, manic depression or bipolar affective disorder, is a psychiatric diagnosis that describes a category of mood disorders defined by the presence of one or more episodes of abnormally elevated mood clinically referred to as mania or, if milder, hypomania. Individuals who experience manic episodes also commonly experience depressive episodes or symptoms, or mixed episodes in which features of both mania and depression are present at the same time. These episodes are usually separated by periods of “normal” mood, but in some individuals, depression and mania may rapidly alternate, known as rapid cycling. Extreme manic episodes can sometimes lead to psychotic symptoms such as delusions and hallucinations. The disorder has been subdivided into bipolar I, bipolar II, cyclothymia, and other types, based on the nature and severity of mood episodes experienced; the range is often described as the bipolar spectrum.

Anxiety Disorders

Anxiety disorder is a blanket term covering several different forms of abnormal and pathological fear and anxiety. Current psychiatric diagnostic criteria recognize a wide variety of anxiety disorders. Recent surveys have found that as many as 18% of Americans may be affected by one or more of them.

Generalized anxiety disorder is a common chronic disorder characterized by long-lasting anxiety that is not focused on any one object or situation. Those suffering from generalized anxiety experience non-specific persistent fear and worry and become overly concerned with everyday matters. Generalized anxiety disorder is the most common anxiety disorder to affect older adults.

In panic disorder, a person suffers from brief attacks of intense terror and apprehension, often marked by trembling, shaking, confusion, dizziness, nausea, difficulty breathing. These panic attacks, defined by the APA as fear or discomfort that abruptly arises and peaks in less than ten minutes, can last for several hours and can be triggered by stress, fear, or even exercise; although the specific cause is not always apparent. In addition to recurrent unexpected panic attacks, a diagnosis of panic disorder also requires that said attacks have chronic consequences: either worry over the attacks' potential implications, persistent fear of future attacks, or significant changes in behavior related to the attacks. Accordingly, those suffering from panic disorder experience symptoms even outside of specific panic episodes. Often, normal changes in heartbeat are noticed by a panic sufferer, leading them to think something is wrong with their heart or they are about to have another panic attack. In some cases, a heightened awareness (hypervigilance) of body functioning occurs during panic attacks, wherein any perceived physiological change is interpreted as a possible life threatening illness (i.e. extreme hypochondriasis).

Obsessive compulsive disorder is a type of anxiety disorder primarily characterized by repetitive obsessions (distressing, persistent, and intrusive thoughts or images) and compulsions (urges to perform specific acts or rituals). The OCD thought pattern may be likened to superstitions insofar as it involves a belief in a causative relationship where, in reality, one does not exist. Often the process is entirely illogical; for example, the compulsion of walking in a certain pattern may be employed to alleviate the obsession of impending harm. And in many cases, the compulsion is entirely inexplicable, simply an urge to complete a ritual triggered by nervousness. In a minority of cases, sufferers of OCD may only experience obsessions, with no overt compulsions; a much smaller number of sufferers experience only compulsions.

The single largest category of anxiety disorders is that of Phobia, which includes all cases in which fear and anxiety is triggered by a specific stimulus or situation. Sufferers typically anticipate terrifying consequences from encountering the object of their fear, which can be anything from an animal to a location to a bodily fluid.

Post-traumatic stress disorder or PTSD is an anxiety disorder which results from a traumatic experience. Post-traumatic stress can result from an extreme situation, such as combat, rape, hostage situations, or even serious accident. It can also result from long term (chronic) exposure to a severe stressor, for example soldiers who endure individual battles but cannot cope with continuous combat. Common symptoms include flashbacks, avoidant behaviors, and depression.

Combination Therapies

In some embodiments, the subject is being treated with an additional therapeutic agent. Such additional agents include atypical antipsychotics such as aripiprazole, clozapine, ziprasidone, risperidone, quetiapine, olanzapine, amisulpride, asenapine, iloperidone, melperone, paliperidone, perospirone, sertindole and sulpiride; and typical antipsychotics such as haloperidol, molindone, loxapine, thioridazine, molindone, thiothixene, pimozide, fluphenazine, trifluoperazine, mesoridazine, chlorprothixene, chlorpromazine, perphenazine, triflupromazine and zuclopenthixol.

Clinical Outcomes

In some embodiments, treatment with a compound or composition described herein, for example, using a method described herein, improves one or more clinical outcomes. For example, in some embodiments, treatment with a compound or composition described herein may improve cognitive function. Elements of cognitive function include memory, orientation, attention, reasoning, language and praxis.

In some embodiments, clinical outcomes may be assessed using known methods. One such method is the Brief Psychiatric Rating Scale (BPRS), a multi-item inventory of general psychopathology traditionally used to evaluate the effects of drug treatment in schizophrenia. The BPRS psychosis cluster (conceptual disorganization, hallucinatory behavior, suspiciousness, and unusual thought content) is considered a particularly useful subset for assessing actively psychotic schizophrenic patients.

In some embodiments, clinical outcomes may be assessed using the 7-point Clinical Global Impression (CGI) rating scale, a commonly used measure of symptom severity, treatment response and the efficacy of treatments. The CGI reflects the impression of a skilled observer, fully familiar with the manifestations of schizophrenia, about the overall clinical state of the patient.

In some embodiments, clinical outcomes may be assessed using the 30-item Positive and Negative Symptoms Scale (PANSS). The name refers to the two types of symptoms in schizophrenia, as defined by the American Psychiatric Association: positive symptoms, which refer to an excess or distortion of normal functions (e.g. hallucinations and delusions), and negative symptoms, which represent a dimunition or loss of normal functions.

In some embodiments, clinical outcomes may be assessed using the Scale for Assessing Negative Symptoms (SANS). SANS assesses five symptom complexes to obtain clinical ratings of negative symptoms in patients with schizophrenia. They are: affective blunting; alogia (impoverished thinking); avolition/apathy; anhedonia/asociality; and disturbance of attention. Assessments are conducted on a six-point scale.

The invention is further illustrated by the following examples which are intended to illustrate but not limit the scope of the invention.

### EXAMPLES

### Abbreviations

### DCM: Dichloromethane

EA, EtOAc or AcOEt: Ethyl acetate
PE: Petroleum ether

### DIPEA: Diisopropylethylamine

### TEA: Triethylamine

rt: Room temperature
SOCl₂: Thionyl chloride
POCl₃: Phosphorous oxychloride

### THF: Tetrahydrofuran

NaOAc: Sodium acetate

### MeOH: Methanol

i-AmOH: Isoamyl alcohol
NaH: Sodium hydride
NaBH₃CN: Sodium cyanoborohydride
n-BuLi: n-Butyl lithium
LHMDS: Lithium bis(trimethylsilyl)amide
LDA: Lithium diisopropylamide
i-PrOH: Isopropyl alcohol
Na₂SO₄: Sodium sulfate
MgSO₄: Magnesium sulfate

### MeCN: Acetonitrile

NaOH: Sodium hydroxide

### EtOH: Ethanol

CuI: Copper(I) iodide
Pd(PPh₃)₂Cl₂: trans-Dichlorobis(triphenylphosphine)palladium(II)
MsCl: Methanesulfonyl chloride
BINAM: [1,1′-Binaphthalene]-2,2′-diamine
XPhos: 2-Dicyclohexylphosphino-2′,4′,6′-triisopropylbiphenyl
SPhos: 2-Dicyclohexylphosphino-2′,6′-dimethoxybiphenyl

### DavePhos: 2-(Dicyclohexylphosphino)-2′-(N,N-dimethylamino)biphenyl

Cs₂CO₃: Cesium carbonate
K₂CO₃: Potassium carbonate
Na₂CO₃: Sodium carbonate

### Mwave or nW or mW: Microwave

t-BuOH: tert-Butanol
K₃PO₄: Potassium phosphate
Pd(APhos)₂Cl₂:Bis(di-tert-butyl(4-dimethylaminophenyl)phosphine)clichloro palladium(II)

### Pd(PPh₃)₄: Tetrakis(triphenylphosphine)palladium (0)

Pd(dppf)₂Cl₂: Dichloro[1,1′-bis(diphenylphosphino)ferrocene]palladium(II)
Pd(OAc)₂ Palladium(II) acetate
Pd₂ dba₃: Tris(dibenzylideneacetone)dipalladium (0)
Pd-118: Dichloro[1,1′-bis(di-t-butylphosphino)ferrocene]palladium(II)
Xantphos: 9,9-Dimethyl-4,5-bis(diphenylphosphino)xanthene
BINAP: (±)-2,2′-B is(diphenylphosphino)-1,1′-binaphthalene
EDCI or EDC: 1-Ethyl-3-(3-dimethylaminopropyl) carbodiimide

### HOBt: Hydroxybenzotriazole

NH₄OH: Ammonium hydroxide

### H₂O: Water

Pd/C: Palladium on carbon

### DMF: N,N-Dimethylformamide

KOCN: Potassium cyanate
WSC-HCl or WSCDI: Water Soluble Carbodiimide hydrochloride
HATU: O-(7-Azabenzotriazol-1-yl)-N,N,N′,N′-tetramethyluronium hexafluorophosphate
HBTU: O-(Benzotriazol-1-yl)-N,N,N′,N′-tetramethyluronium hexafluorophosphate
Py-Brop: Bromotripyrrolidinophosphonium hexafluorophosphate
BOP: Benzotriazole-1-yl-oxy-tris-(dimethylamino)-phosphonium hexafluoro phosphate
DBU: Diaza(1,3)bicyclo[5.4.0]undecene
DMSO: Dimethyl sulfoxide
LCMS: Liquid chromatography mass spectrometry
HPLC: High performance liquid chromatography

### DMA: N,N-dimethylacetamide

h: hour
TLC: Thin layer chromatography
TFA: Trifluoroacetic acid

### Et₃N: Triethylamine

### DIPEA: N,N-Diisopropylethylamine

### O.N: Overnight

TBSO: tert-Butyldimethylsilyloxy

### DME: Dimethoxyethane

NMP: 1-Methyl-2-pyrrolidinone
PS-BEMP: 2-tert-Butylimino-2-diethylamino-1,3-dimethylperhydro-1,3,2-diazaphosphorine supported on Polystyrene
PBr₃: Phosphorus tribromide
NaOtBu: Sodium tert-butoxide
KI: Potassium iodide

### PPh₃: Triphenylphosphine

### NMM: N-Methylmorpholine

### HCHO: Formaldehyde

PG: Protecting group
ISCO: Teledyne ISCO purification systems
BINAM: 1,1′-Binaphthyl-2,2′-diamine.
DABCO: 1,4-Diazabicyclo[2.2.2]octane
Ac₂O: Acetic anhydride

### N₂: Nitrogen gas

NaHCO₃: Sodium bicarbonate
NaNO₂: Sodium nitrite

### Ar: Argon gas

### General Experimental

All exemplified target compounds are fully analyzed and characterized (TLC, LCMS, ¹H-NMR) prior to submission for biological evaluation. Thin-layer chromatography was carried out on native silica 254F plates. Visualization was accomplished with ultraviolet or phosphomolybdic acid. ¹H-NMR spectra were recorded on multiple NMR spectrometers, either on 400 MHz on a Avance III 400 Ultra shield-plus TM digital Spectrometer or on 300 MHz using a Varian Mercury 300Plus Spectrometer, designated by 400 MHz or 300 MHz, respectively. ¹H-NMR spectra were also recorded on a Bruker Spectrospin 300 MHz Spectrometer at 300.13 MHz in DMSO-d₆ with TMS as an internal standard and will be designated as Bruker 300 Hz. NMR assignments are based on a combination of the ¹H, ¹³C, ¹HCOSY, HMBC and HMQC spectra. Coupling constants are given in hertz (Hz). Anhydrous methylene chloride, tetrahydrofuran, and dimethylformamide were obtained by distillation, and other materials are reagent grade.

LC-MS Methods are listed here:

### Method A

Mobile phase: A=0.1% TFA/H₂O, B=0.01% TFA/MeCN; Gradient: B=5%-95% in 1.5 min; Flow rate: 2.0 mL/min; Column: sunfire-C₁₈, 50×4.6 mm, 3.5 um;

### Method B

Mobile phase: A=10 mM NH₄HCO₃/H₂O, B=MeCN; Gradient: B=5%-95% in 1.5 min; Flow rate: 2.0 mL/min; Column: Xbridge-C₁₈, 50×4.6 mm, 3.5 um;

### Method C

Mobile phase: A=10 mM ammonium formate/H₂O/4.9% MeCN, B=MeCN; Gradient: B=5%-100% in 2.0 min; Flow rate: 2.5 mL/min; Column: Atlantis T3 3 uM 4.6×30 mm

### Method D

Mobile phase: A=0.1% formic acid/H₂O/4.9% MeCN, B=MeCN; Gradient: B=5%-100% in 2.0 min; Flow rate: 2.5 mL/min; Column: Atlantis T3 3 uM 4.6×30 mm

### Method E

Mobile phase: A=0.05% TFA/H₂O, B=0.05% TFA/MeCN; Gradient: B=5%-100% in 3.0 min; Flow rate: 0.8 mL/min; Column: CAPCELL PAK C18 (Shiseido, UG120, 3 mM, 2.0 mm I.D.×50 mm)

Representative Conditions of PREP-HPLC are listed here:

### PREP-HPLC Condition A (Basic Mobile Phase):

Instrument: Gilson 281

Mobile Phase: A=0.01% NH₄HCO₃/H₂O, B=MeCN

Flow Rate: 40.0 mL/min

Column: AGT Venusil XBP C₁₈, 10.0 um, 30 mm×100 mm

### PREP-HPLC Condition B (Basic Mobile Phase):

Instrument: Gilson 281

Mobile Phase: A=NH₃—H₂O, 10 mmol/L, B=MeCN

Flow Rate: 40.0 mL/min

Column: Waters X-Bridge, 5.0 um, 30 mm×150 mm

### PREP-HPLC Condition C (Basic Mobile Phase):

Instrument: Gilson 281

Mobile Phase: A=0.01% NH₄HCO₃/H₂O, B=MeCN

Flow Rate: 30.0 mL/min

Column: Shimadzu PRC-ODS, 10.0 um, 20 mm×250 mm

Gradient: B=xx %-yy % 0.0 to 8.0 min
yy %-95% 8.0 to 8.2 min
95%-95% 8.2 to 11.0 min

The following table shows the relationship of representative value (xx %-yy %) of gradient and retention time on LC-MS of corresponding compound.

25%-30% 0.5-1.0 min
30%-50% 1.0-1.5 min
50%-70% 1.5-1.75 min
70%-90% 1.7-2.0 min

### PREP-HPLC Condition D:

Instrument: Waters 600 pump, Waters 2996, Photodiode Array Detector, Waters Micromass ZQ, Gilson 215 Liquid Handler.

Mobile Phase: A=0.05% TFA/H₂O, B=MeCN

Flow Rate: 36.0 mL/min

Column: Shiseido CAPCELL PAK C18, UG120, 5 uM, 20 mm I.D.×50 mm

Gradient: B=5%-100% 0.0 to 4.0 min

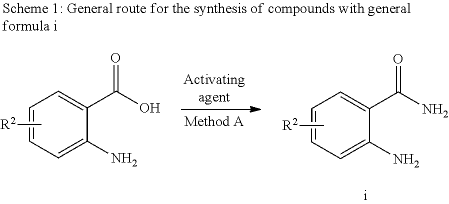

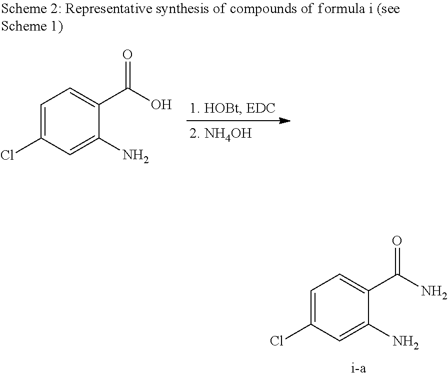 [SMILES: Nc1cc(Cl)ccc1C(=O)O ; NC(=O)c1ccc(Cl)cc1N]

### Method A: 2-Amino-4-chlorobenzamide (1-a)

To a mixture of 2-amino-4-chlorobenzoic acid (3.42 g, 20 mmol) in DMF (45 mL) was added HOBt (2.70 g, 20 mmol). After stirring for 10 min, EDC hydrochloride (3.82 g, 20 mmol) was added to the mixture. The resulted mixture was stirred at room temperature for 2 h. NH₄OH (28%, 5 mL) was added at 0° C. with vigorous stirring. After addition, the mixture was stirred at room temperature for another 2 h. The reaction mixture was added to water (200 mL) dropwise with stirring, then a precipitate formed. The precipitate was collected and dried in vacuo to give 2.98 g of i-a as a grey solid (87.6% yield). LCMS m/z=171.0 (M+1), 173.0 (M+3) (Method B) (retention time=1.39 min) ¹H NMR (400 MHz, DMSO-d₆): δ 7.27 (d, J=9.6 Hz, 1H), 6.68 (d, J=2.4 Hz, 1H), 6.60 (dd, J=8.4, 2.0 Hz, 1H), 5.50-5.82 (m, 4H).

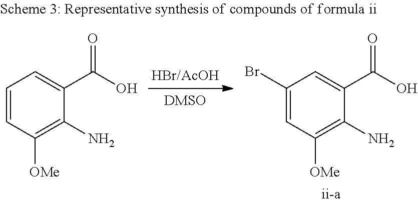 [SMILES: COc1cccc(C(=O)O)c1N ; COc1cc(Br)cc(C(=O)O)c1N]

### Method B: 2-Amino-5-bromo-3-methoxybenzoic acid (ii-a)

To the solution of 2-amino-3-methoxybenzoic acid (10.0 g, 60 mmol) in DMSO (80 mL) was added HBr (33% in HOAc, 40 mL) dropwise. The resulting solution was stirred overnight and then poured into water (600 mL). The precipitate was collected as the target product 2-amino-5-bromo-3-methoxybenzoic acid 14.1 g in a yield of 96%. LCMS m/z=246.0, 248.0 (M+1) (method B) (Retention time=1.159 min)

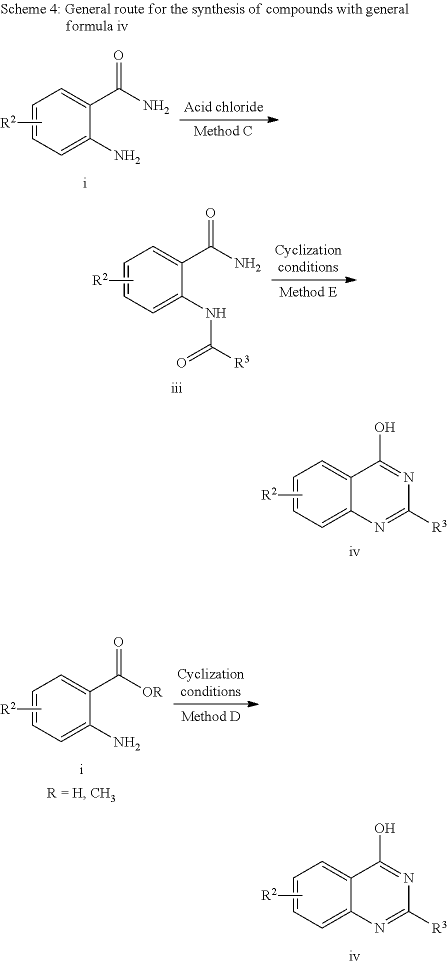

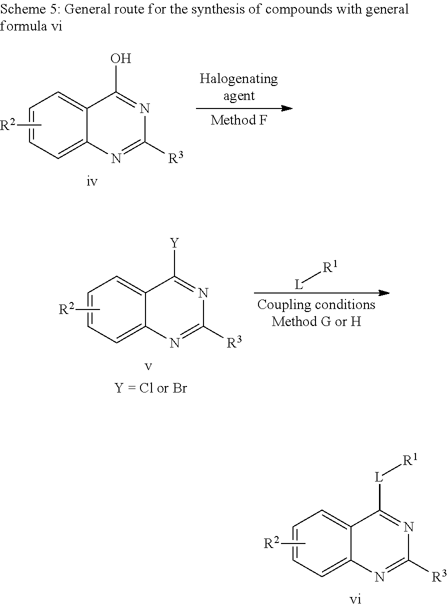

### Method C for Coupling Condition:

### C1: CH₂Cl₂/TEA

### C2: Pyridine/THF

### Method F for Chlorinating Conditions

### F1: SOCl₂/DMF/80° C.

### F2: POCl₃/Δ

### F3: POCl₃/Toluene/100° C.

### F4: PBr₃/CH₂Cl₂/DMF/60° C.

### Method G for Coupling Conditions

G1: i-PrOH/0.1 N HCl/85-100° C.

### G2: NaH/DMF

### G3: K₂CO₃/DMF/60° C.

### Method H for Coupling Conditions

H1: Pd₂(dba)₃/Xantphos/Cs₂CO₃/Dioxane/85-100° C.
H2: Pd₂(dba)₃/BINAP/NaO^{t}Bu/Dioxane/60° C.

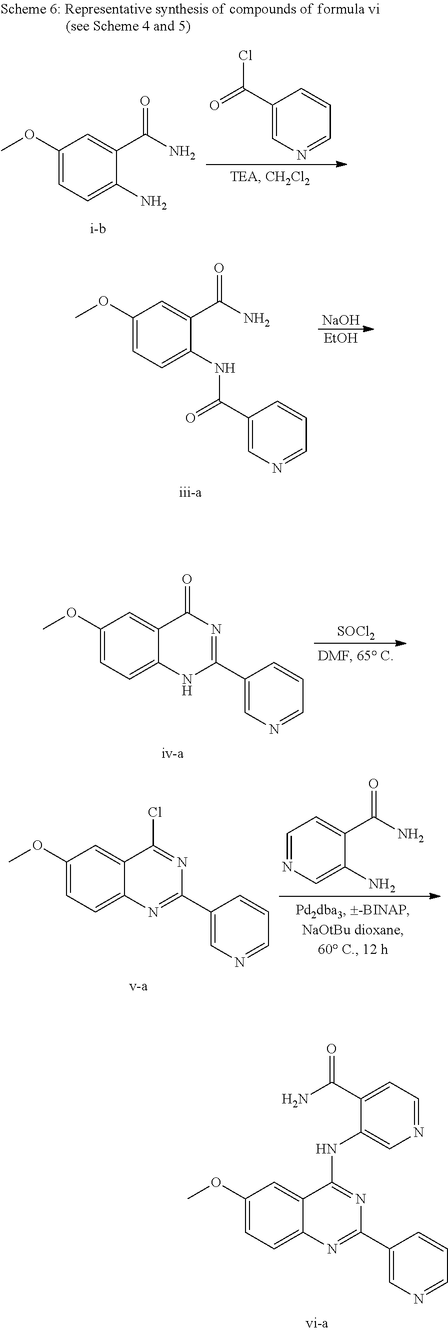

### Method C1: N-(2-carbamoyl-4-methoxyphenyl)nicotinamide (iii-a)

To a 250 mL round-bottomed flask were added 2-amino-5-methoxybenzamide (1.900 g, 11.43 mmol) and nicotinoyl chloride hydrochloride (2.035 g, 11.43 mmol) in CH₂Cl₂ (50 mL). The mixture was cooled to 0° C., and triethylamine (4.35 mL, 31.2 mmol) was added dropwise with stirring. The reaction was then allowed to warm to ambient temperature and proceed overnight. After the reaction was complete, the resulting precipitate was filtered and washed with dichloromethane, water and ether to yield the title compound as a white solid (2.14 g, 7.5 mmol, 76%). LC-MS m/z=272.1 (M+1) (retention time=1.31).

### Method C2: N-(2-carbamoyl-4-methoxyphenyl)nicotinamide (iii-a)

To a round-bottomed flask was added 2-amino-5-methoxybenzamide (28.3 g, 170 mmol) and nicotinoyl chloride hydrochloride (31.8 g, 179 mmol) in THF (300 mL). The mixture was cooled to 0° C., and pyridine (55.1 mL, 681 mmol) was added dropwise with stirring. The reaction was then allowed to warm to ambient temperature and proceed overnight. After the reaction was completed, the volatiles were removed under vacuum. The solid residue was crushed and water (300 mL), MeOH (100 mL) and NH₃aq (20 mL) were added. The mixture was stirred for 15 min, the solidified compound was filtered off, and washed with MeOH-water. The compound was dried to give the title compound as a pale yellow powder. (45.9 g, 99%). ¹H NMR (400 MHz, DMSO) δ12.69 (s, 1H), 9.09 (dd, J=2.4, 0.9 Hz, 1H), 8.79 (dd, J=4.8, 1.6 Hz, 1H), 8.54 (d, J=9.1 Hz, 1H), 8.44 (s, 1H), 8.25 (ddd, J=8.0, 2.4, 1.7 Hz, 1H), 7.87 (s, 1H), 7.62 (ddd, J=8.0, 4.8, 0.9 Hz, 1H), 7.46 (d, J=2.9 Hz, 1H), 7.19 (dd, J=9.1, 2.9 Hz, 1H), 3.82 (s, 3H).

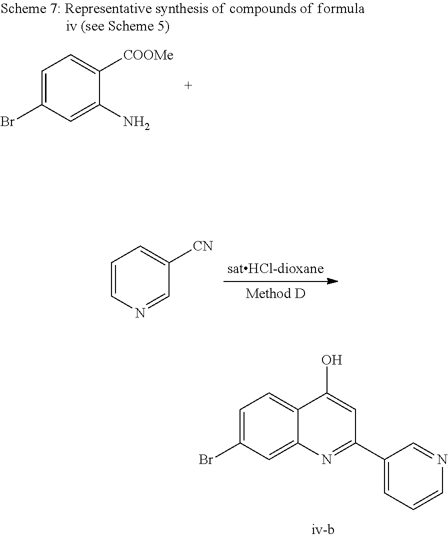 [SMILES: COC(=O)c1ccc(Br)cc1N ; "+" ; N#Cc1cccnc1 ; Oc1cc(-c2cccnc2)nc2cc(Br)ccc12]

### Method D: 7-Bromo-2-(pyridin-3-yl)quinazolin-4-ol (iv-b)

A 3 L round-bottom flask was charged with methyl 2-amino-4-bromobenzoate (100 g, 435 mmol) and 3-cyanopyridine (91 g, 869 mmol) and cooled in an ice bath. A saturated solution of HCl in 1,4-dioxane (1.2 L) was added. The reaction was stirred at room temperature for 3 days and then diluted with diethyl ether (1.2 L) to precipitate the product. The precipitate was filtrated and washed with diethyl ether (500 mL). The crude material including 7-bromo-4-methoxy-2-(pyridin-3-yl)quinazoline and 7-bromo-2-(pyridin-3-yl)quinazolin-4-ol was placed into a round-bottom flask, then EtOH (1 L) and H₂O (1 L) were added, followed by a 50w/v % NaOH solution (200 mL) at 0° C. The reaction was allowed to warm to 65° C. and stirred for 5 h, the 4-methoxy quinazoline derivative was completely cleaved to the desired product. The solvent was concentrated to a minimal amount and then 1 L of ethanol was added to the solution and the desired product precipitated. The product was filtered to give 7-bromo-2-(pyridin-3-yl)quinazolin-4-ol as the sodium salt. The salt was neutralized by suspending in 2 L of ethanol (2 L) with cooling in an ice-bath, then Ac₂O (200 mL) was added slowly. The product was collected by filtration and washed with ethanol and dried at 60° C. to give 7-bromo-2-(pyridin-3-yl)quinazolin-4-ol as a white powder (120 g, 92%). ¹H NMR (300 MHz, DMSO) δ 12.86 (brs, 1H), 9.29 (d, J=2.2 Hz, 1H), 8.77 (dd, J=4.8, 1.5 Hz, 1H), 8.63-8.39 (m, 1H), 8.07 (d, J=8.5 Hz, 1H), 7.96 (d, J=1.8 Hz, 1H), 7.70 (dd, J=8.5, 1.9 Hz, 1H), 7.60 (dd, J=8.0, 4.8 Hz, 1H).

### 6-bromo-2-(pyridin-3-yl)quinazolin-4-ol (iv-c)

In a 350 mL sealed tube was added 3-cyanopyridine (2.67 g, 25.6 mmol) and methyl 2-amino-5-bromobenzoate (5.90 g, 25.6 mmol) in 4M hydrogen chloride in 1,4-dioxane (100 ml, 400 mmol). The mixture was allowed to stir for 48 h at 120° C. After cooling to room temperature, the precipitate was collected by filtration and subsequently washed with dioxane, methanol and ether. The isolated hydrochloride salt was taken up in water (150 mL) and basified with NH₄OH solution to pH 8. The resultant precipitate was collected by filtration, washed with water, methanol and ether and dried to give the crude product that was recrystallized from ethanol to provide 5.72 g of 6-bromo-2-(pyridin-3-yl)quinazolin-4-ol as a white solid (74%). LC-MS m/z=302.3 (M+1) (Method C) (retention time=1.59 min)

### Method E: 6-Methoxy-2-(pyridin-3-yl)quinazolin-4-ol (iv-a)

A mixture of N-(2-carbamoyl-4-methoxyphenyl)nicotinamide (2.40 g, 8.8 mmol, 1.0 eq) in EtOH (60 mL) was treated with NaOH (1.76 g, 44 mmol, 5.0 eq). The resulting mixture was stirred at room temperature overnight. After the reaction was completed, the volatiles were removed in vacuo. Water (100 mL) was added to the residue and the mixture was adjusted to pH ˜5 or 6 by slow addition of aqueous HCl (4N). The resulting precipitate was collected and dried to give 2.20 g of 6-methoxy-2-(pyridin-3-yl)quinazolin-4-ol as a yellow solid (98.6% yield). LCMS m/z=254.1 (M+1) (Method B) (retention time=1.336 min)

### Method F1: 4-Chloro-6-methoxy-2-(pyridin-3-yl)quinazoline (v-a)

6-methoxy-2-(pyridin-3-yl)quinazolin-4-ol (1.20 g, 4.74 mmol) and catalytic DMF was added to SOCl₂ (10 mL). The resulting mixture was stirred at 65° C. for 2 h. After the reaction was complete and cooled, the mixture was carefully poured into ice-water. The pH was adjusted to 7 by slow addition of NH₄OH at 0° C. The resulting solid was collected and dried to give 900 mg of 4-chloro-6-methoxy-2-(pyridin-3-yl)quinazoline as a beige solid (quantitative yield). LCMS m/z=271.9 (M+1) (Method A) (retention time=1.610 min).

### Method F2: 4-Chloro-6-methoxy-2-(pyridin-3-yl)quinazoline (v-a)

In a sealed tube, phosphorus oxychloride (11 mL, 120 mmol) was added to 6-methoxy-2-(pyridin-3-yl)quinazolin-4(3H)-one (2.70 g, 10.66 mmol). The mixture was heated at 120° C. for 12 h. After cooling, the remaining phosphorus oxychloride was removed in vacuo to leave a tan solid. This residue was added to an ice-water mixture (100 mL) with cooling and allowed to stir. The pH of the suspension was adjusted to about pH 9 via dropwise addition of 28% ammonium hydroxide, and stirring was continued for 30 mins. The resulting solid was filtered to give the desired product as a tan solid (2.55 g, 9.39 mmol, 88%). LC-MS m/z=272.0 (M+1) (retention time=2.05) ¹H NMR (300 MHz, DMSO) δ 9.55 (s, 1H), 8.81-8.64 (m, 2H), 8.09 (d, J=9.2 Hz, 1H), 7.78 (dd, J=9.2, 2.8 Hz, 1H), 7.61 (dd, J=7.9, 4.8 Hz, 1H), 7.49 (d, J=2.5 Hz, 1H), 4.00 (s, 3H).

### Method F3: 4-chloro-6-ethoxy-2-(pyridin-3-yl)quinazoline (v-b)

To a suspension of 6-ethoxy-2-(pyridin-3-yl)quinazolin-4-ol (34 g, 0.127 mol) in toluene (50 mL) was added phosphorus oxychloride (47.4 mL, 0.509 mol) at room temperature. The mixture was refluxed for 6 h. The solvent was evaporated and water was added to the residue under cooling conditions. The mixture was neutralized to pH 7 by slow addition of NaOHaq, and extracted with CH2Cl2. The combined organic layer was washed with water and brine and was dried over Na2SO₄. After filtration and evaporation, the crude product was purified by column chromatography on NH-silica gel (eluted with CH2Cl2) to give the title compound as a white powder. (33.2 g, 91%). ¹H NMR (400 MHz, CDCl₃) δ9.74 (dd, J=2.2, 0.9 Hz, 1H), 8.80 (ddd, J=8.0, 2.3, 1.7 Hz, 1H), 8.72 (dd, J=4.8, 1.7 Hz, 1H), 8.02 (d, J=9.2 Hz, 1H), 7.60 (dd, J=9.2, 2.8 Hz, 1H), 7.41-7.48 (m, 2H), 4.24 (q, J=7.0 Hz, 2H), 1.53 (d, J=7.0 Hz, 3H).

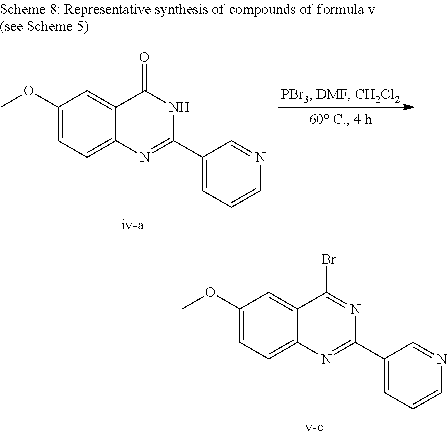 [SMILES: COc1ccc2nc(-c3cccnc3)[nH]c(=O)c2c1 ; COc1ccc2nc(-c3cccnc3)nc(Br)c2c1]

### Method F4: 4-Bromo-6-methoxy-2-(pyridin-3-yl)quinazoline (v-c)

To a sealed tube containing 6-methoxy-2-(pyridin-3-yl)quinazolin-4(3H)-one (1.30 g, 5.13 mmol) and dichloromethane (20 mL) were added 1M phosphorus tribromide in dichloromethane (10.3 mL, 10.3 mmol) and DMF (2 mL). The reaction mixture was heated at 60° C. for 4 h. After cooling, excess dichloromethane was evaporated leaving a tan residue. The solid was added to an ice-water mixture (100 mL) with cooling and allowed to stir at room temperature. The pH of the suspension was adjusted to about pH 9 via dropwise addition of 28% ammonium hydroxide, and stirring was continued for 30 mins. The resulting solid was filtered to give the desired product as a tan solid (1.49 g, 4.71 mmol, 92%). LC-MS m/z=318.3 (M+2) (retention time=2.19).

### Method G1: N-(6-chloropyridin-2-yl)-6-methoxy-2-(pyridin-3-yl)quinazolin-4-amine (vi-b)

A mixture of 4-chloro-6-methoxy-2-(pyridin-3-yl)quinazoline (300 mg, 1.10 mmol) and 6-chloropyridin-2-amine (568 mg, 4.40 mmol) in 0.5N HCl/1-PrOH (10 mL) was stirred at 85° C. for 7 h. The yellow precipitate was collected and washed with i-PrOH. The solid was recrystallized from MeOH to give 49 mg of vi-b as a yellow powder as the HCl salt (10%). ¹H NMR (400 MHz, DMSO) δ 10.95 (s, 1H), 9.58 (d, J=1.7 Hz, 1H), 9.13 (d, J=8.1 Hz, 1H), 8.92 (d, J=5.2 Hz, 1H), 8.48 (d, J=8.2 Hz, 1H), 8.22 (d, J=2.7 Hz, 1H), 8.06-7.97 (m, 2H), 7.95 (d, J=9.1 Hz, 1H), 7.63 (dd, J=9.1, 2.7 Hz, 1H), 7.33 (d, J=7.2 Hz, 1H), 4.00 (s, 3H).

### Method G2: 6-methoxy-2-(pyridin-3-yl)-4-(1H-pyrrolo[3,2-c]pyridin-1-yl)quinazoline (vi-c)

To a round bottom flask was first added sodium hydride 60% (57.8 mg, 1.32 mmol) and 1H-pyrrolo[3,2-c]pyridine (157 mg, 1.32 mmol) in DMF (15 mL). The mixture was allowed to stir at room temperature for 10 min. Then, 4-chloro-6-methoxy-2-(pyridin-3-yl)quinazoline (300 mg, 1.10 mmol) was added to the mixture, and the reaction was allowed to proceed at room temperature overnight. Water (50 mL) was added to the mixture, and the resultant precipitate was collected by filtration. The crude product was purified via NH-silica gel chromatography (Ethyl acetate/hexane=25% to 75%) to afford 316 mg of the desired product as a white solid (81%). The resulting product was converted to the di HCl salt using HCl_{(aq)}/EtOH. ¹H NMR (400 MHz, DMSO) δ 9.68 (d, J=1.6 Hz, 1H), 9.16-9.11 (m, 1H), 9.00-8.92 (m, 3H), 8.89 (dd, J=5.6, 1.0 Hz, 1H), 8.28 (d, J=9.2 Hz, 1H), 7.96 (dd, J=8.1, 5.2 Hz, 1H), 7.90 (dd, J=9.2, 2.7 Hz, 1H), 7.85 (dd, J=8.4, 5.6 Hz, 1H), 7.40 (d, J=2.7 Hz, 1H), 7.35 (dd, J=3.6, 0.7 Hz, 1H), 3.91 (s, 3H).

### Method G3: N-(4-chloropyridin-2-yl)-6-methoxy-2-(pyridin-3-yl)quinazolin-4-amine (vi-d)

To a suspension of 4-chloro-6-methoxy-2-(pyridin-3-yl)quinazoline (300 mg, 1.10 mmol) and 4-chloropyridin-2-amine (156 mg, 1.22 mmol) in DMF (20 mL) was added Cs₂CO₃ (432 mg, 1.33 mmol) at room temperature. The mixture was stirred at 60° C. for 1 h. Water was added and a precipitate formed which was collected by filtration and washed with H₂O. The crude product was purified via NH-silica gel chromatography (Ethyl acetate/hexane=25% to 80%) to afford 9 mg of the desired product as a white powder (2%), ¹H NMR (400 MHz, DMSO) δ 10.82 (s, 1H), 9.55 (dd, J=2.1, 0.8 Hz, 1H), 8.76 (d, J=1.7 Hz, 1H), 8.71-8.66 (m, 2H), 8.46 (d, J=5.4 Hz, 1H), 8.19 (d, J=2.7 Hz, 1H), 7.89 (d, J=9.1 Hz, 1H), 7.60-7.55 (m, 2H), 7.34 (dd, J=5.4, 1.9 Hz, 1H), 3.98 (s, 3H).

### Method H2: 3-(6-methoxy-2-(pyridin-3-yl)quinazolin-4-ylamino)isonicotinamide, 3HCl (vi-a) (This method is representative of method H1 can be implemented in a similar way except for substitution of the appropriate catalyst and base)

To a 1 dram reaction vial was added 4-bromo-6-methoxy-2-(pyridin-3-yl)quinazoline (0.150 g, 0.474 mmol), 3-amino-isonicotinamide (0.072 g, 0.522 mmol), tris(dibenzylideneacetone)dipalladium (0) (0.022 g, 0.024 mmol), racemic-BINAP (0.030 g, 0.047 mmol), and sodium tert-butoxide (0.137 g, 1.423 mmol) in dioxane (1.5 ml) to give a brown suspension. The reaction was heated at 60° C. overnight. Upon cooling, water (50 mL) was added to the reaction mixture, and the crude product was extracted with ethyl acetate (5×75 mL). The combined organics were dried (Na₂SO₄), filtered and concentrated. This material was then purified via ISCO (silica gel, 91:9 CH₂Cl₂/MeOH, 4 gm column). The fractions collected were concentrated and dried under vacuum to give a yellow powder. To form the salt, the material was suspended in methanol prior to the addition of 4 M HCl in dioxane. After stirring at ambient temperature for 2 h, the resulting precipitate was filtered to give the title compound as a yellow solid (24.7 mg, 0.051 mmol, 25%). LC-MS m/z=373.4 (M+1) (retention time=1.64) ¹H NMR (300 MHz, DMSO) δ 12.06 (s, 1H), 9.84 (s, 1H), 9.54 (d, J=1.6 Hz, 1H), 9.05 (d, J=7.5 Hz, 1H), 8.89 (d, J=5.1 Hz, 1H), 8.58 (t, J=5.3 Hz, 2H), 8.11 (d, J=1.0 Hz, 1H), 8.02-7.91 (m, 2H), 7.87 (d, J=5.3 Hz, 1H), 7.71 (d, J=1.8 Hz, 1H), 7.65 (dd, J=8.5, 2.8 Hz, 1H), 3.98 (s, 3H).

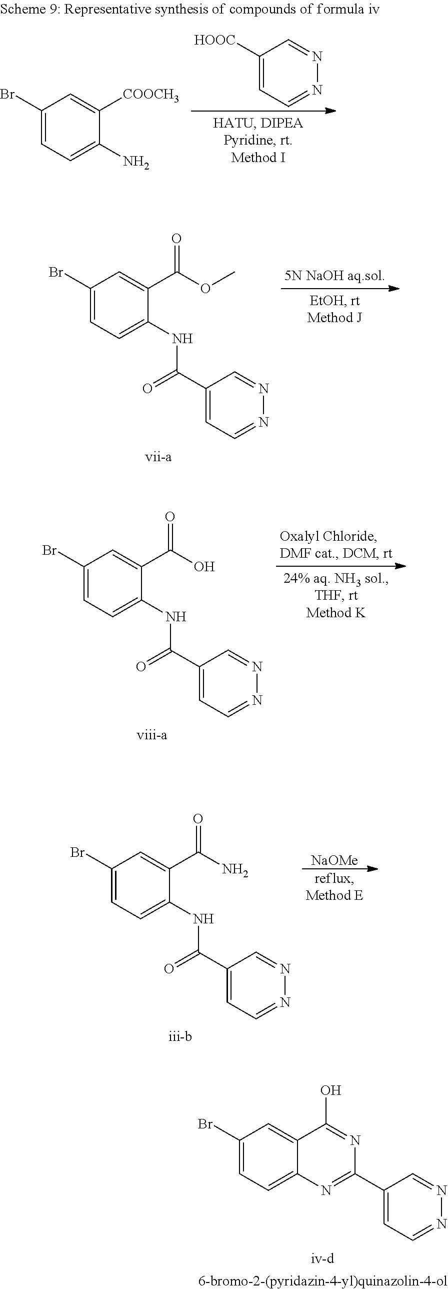

### Method I: Methyl 5-bromo-2-(pyridazine-4-carboxamido)benzoate (vii-a)

To a suspension of 4-pyridazinecarboxylic acid (4.9 g, 39.5 mmol) in pyridine (100 mL) was added DIPEA (13.8 mL, 79 mmol) and HATU (18 g, 47.4 mmol) under ice cooling. The reaction mixture was stirred at room temperature for 2-3 h, and then methyl 2-amino-5-bromobenzoate (10.9 g, 47.4 mmol) was added. The reaction mixture continued to stir at room temperature overnight. The reaction mixture was poured over crushed ice and stirred at room temperature for 2-3 h. The precipitated product was collected by filtration, washed with water and dried to give methyl 5-bromo-2-(pyridazine-4-carboxamido)benzoate (12 g, 90% yield,) as a colorless solid. ¹H NMR (400 MHz, DMSO) δ 11.43 (s, 1H), 9.63 (dd, J=2.3, 1.2 Hz, 1H), 8.16 (d, J=8.8 Hz, 1H), 8.10-8.05 (m, 2H), 7.91 (dd, J=8.8, 2.4 Hz, 1H).

### Method J: 5-Bromo-2-(pyridazine-4-carboxamido)benzoic acid hydrochloride (viii-a)

Methyl 5-bromo-2-(pyridazine-4-carboxamido)benzoate 1a (12 g, 35 7 mmol) was dissolved in ethanol (100 mL) and 5N aq. NaOH sol (21.4 mL, 107 mmol) and cooled in an ice bath. The reaction mixture was stirred at room temperature for 4 h and checked by LC-MS, no starting material remained Ethanol was removed under vacuum and then diluted with water (200 mL) with cooling in an ice bath. The aqueous solution was acidified with 6N aq. HCl solution to pH 1-2 and a precipitate formed. The solid was collected by filtration, washed with water followed by ethyl acetate (100 mL) and dried at 60° C. for 24 h to afford 5-bromo-2-(pyridazine-4-carboxamido)benzoic acid hydrochloride with a small amount of 2-amino-5-bromobenzoic acid (10 g, 78% yield) as pale brown solid. The compound was used directly in the next step without further purification. ¹H NMR (400 MHz, DMSO) δ 12.15 (s, 1H), 9.63 (dd, J=2.4, 1.2 Hz, 1H), 9.55 (dd, J=5.3, 1.2 Hz, 1H), 8.45 (d, J=8.9 Hz, 1H), 8.13 (d, J=2.5 Hz, 1H), 8.07 (dd, J=5.3, 2.4 Hz, 1H), 7.89 (dd, J=8.9, 2.5 Hz, 1H).

### Method K: N-(4-bromo-2-carbamoylphenyl)pyridazine-4-carboxamide (iii-b)

To a suspension of 5-bromo-2-(pyridazine-4-carboxamido)benzoic acid hydrochloride (10 g) in dichloromethane (200 mL) was added oxalyl chloride (11 mL) with cooling, followed by a few drops of DMF. The reaction mixture was stirred at room temperature for 2 h. Then the reaction mixture was concentrated. The acid chloride intermediate was dissolved in 150 ml of THF, and added portionwise to a cold solution of 25% aq NH₃ (22 mL) in THF (50 mL). [Caution! Proper care should be taken with the addition of the acid chloride to the aqueous ammonia solution due to its exothermic nature, particularly in large scale reactions.] The reaction was stirred at room temperature overnight, and then diluted with water. The organic solvent was removed under vacuum resulting in a precipitate. The precipitate was filtered, washed with water and dried. The crude compound was recrystallized from a methanol-water mixture and then filtered and dried to give N-(4-bromo-2-carbamoylphenyl)pyridazine-4-carboxamide (8 g, 98% yield) to give as a white solid.

¹H NMR (400 MHz, DMSO) δ 13.10 (s, 1H), 9.67-9.39 (m, 2H), 8.60-8.50 (m, 2H), 8.14 (d, J=2.3 Hz, 1H), 8.03 (dd, J=5.3, 2.4 Hz, 2H), 7.82 (dd, J=8.9, 2.2 Hz, 1H).

### 6-bromo-2-(pyridazin-4-yl)quinazolin-4-ol (iv-d)

6-bromo-2-(pyridazin-4-yl)quinazolin-4-ol was synthesized in a manner analogous to that described in Method E substituting N-(4-bromo-2-carbamoylphenyl)pyridazine-4-carboxamide (8 g, 25 mmol) for N-(2-carbamoyl-4-methoxyphenyl)nicotinamide to give 6-bromo-2-(pyridazin-4-yl)quinazolin-4-ol (4 g) in 53% yield. ¹H NMR (400 MHz, DMSO) δ 13.13 (s, 1H), 9.86 (dd, J=2.4, 1.2 Hz, 1H), 9.50 (dd, J=5.4, 1.2 Hz, 1H), 8.33 (dd, J=5.4, 2.4 Hz, 1H), 8.28 (d, J=2.3 Hz, 1H), 8.05 (dt, J=6.8, 3.4 Hz, 1H), 7.78 (d, J=8.7 Hz, 1H).

The compounds in the following table were prepared in a manner analogous to that described in Scheme 1-9 (prepared according to method procedure A-K as designated).

TABLE 1

| | | | | | | | Puri- | | | | |
|---|---|---|---|---|---|---|---|---|---|---|---|
| | | | | | | ¹H | ty | | | Reten- | |
| Num- | Starting | Starting | | Salt | | NMR | per- | Method | | tion | LCMS |
| ber | Material 1 | Material 2 | Product | type | ¹H NMR | Solvent | cent | of Coupling | LCMS | Time | Method |
| 1 | 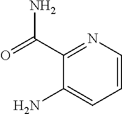 | 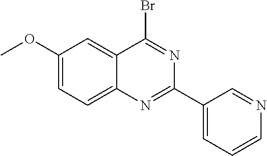 | 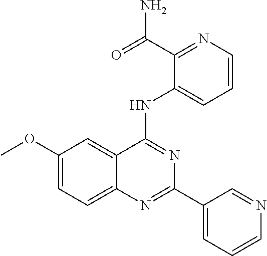 [SMILES: COc1ccc2nc(-c3cccnc3)nc(Nc3cccnc3C(N)=O)c2c1] | 3 HCl | 1H NMR (300 MHz, DMSO) δ 13.63 (s, 1H), 9.65 (s, 1H), 9.58 (dd, J = 8.6, 1.2 Hz, 1H), 9.29 (d, J = 6.9 Hz, 1H), 8.99 (s, 1H), 8.77 (s, 1H), 8.41 (d, J = 3.7 Hz, 1H), 8.32 (s, 1H), 8.12 (dd, J = 7.3, 6.0 Hz, 1H), 7.99 (d, J = 9.1 Hz, 1H), 7.80 (dd, J = 8.7, 4.1 Hz, 1H), 7.67 (dd, J = 8.3, 1.5 Hz, | DMSO | 94 | F4, H2 | 373.4 (M + 1) | 2.08 | C |
| | | | | | 1H), 7.61 (d, J = | | | | | | |
| | | | | | 1.9 Hz, 1H), 7.43- | | | | | | |
| | | | | | 7.26 (m, 2H), | | | | | | |
| | | | | | 3.99 (s, 3H). | | | | | | |
| 2 | 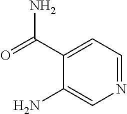 | 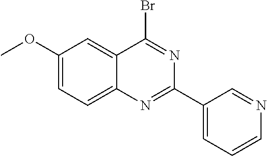 | 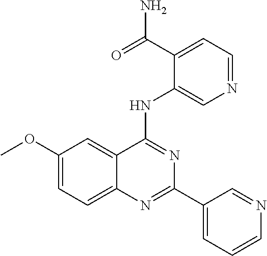 [SMILES: COc1ccc2nc(-c3cccnc3)nc(Nc3cnccc3C(N)=O)c2c1] | 3 HCl | 1H NMR (300 MHz, DMSO) δ 12.06 (s, 1H), 9.84 (s, 1H), 9.54 (d, J = 1.6 Hz, 1H), 9.05 (d, J = 7.5 Hz, 1H), 8.89 (d, J = 5.1 Hz, 1H), 8.58 (t, J = 5.3 Hz, 2H), 8.11 (d, J = 1.0 Hz, 1H), 8.02-7.91 (m, 2H), 7.87 (d, J = 5.3 Hz, 1H), 7.71 (d, J = 1.8 Hz, 1H), 7.65 (dd, J = 8.5, 2.8 Hz, 1H), 3.98 (s, 3H). | DMSO | 97 | F4, H2 | 373.4 (M + 1) | 1.64 | C |
| 3 | 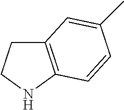 | 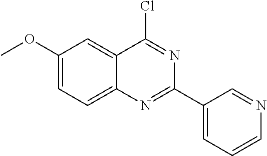 [SMILES: COc1ccc2nc(-c3cccnc3)nc(Cl)c2c1] | 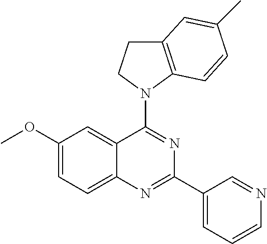 [SMILES: COc1ccc2nc(-c3cccnc3)nc(N3CCc4cc(C)ccc43)c2c1] | 2 HCl | 1H NMR (400 MHz, DMSO) δ 9.55 (s, 1H), 9.13 (d, J = 7.4 Hz, 1H), 8.97 (d, J = 5.3 Hz, 1H), 8.14-8.02 (m, 1H), 7.99 (d, J = 9.1 Hz, 1H), 7.68 (dd, J = 9.1, 2.7 Hz, 2H), 7.56 (d, J = 2.5 Hz, 1H), 7.23 (s, 1H), 7.11 (d, J = 7.5 Hz, 1H), 4.69 (t, J = 7.7 Hz, 2H), 3.91 (d, J = 6.5 Hz, 3H), 3.22 (t, J = | DMSO | >98 | Method C1, E, F3, G2 | | | |
| | | | | | 7.7 Hz, 2H), | | | | | | |
| | | | | | 2.34 (s, 3H). | | | | | | |
| 4 | 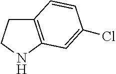 | 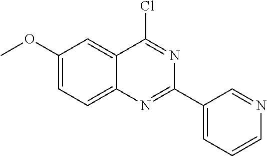 [SMILES: COc1ccc2nc(-c3cccnc3)nc(Cl)c2c1] | 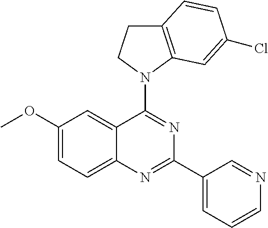 [SMILES: COc1ccc2nc(-c3cccnc3)nc(N3CCc4ccc(Cl)cc43)c2c1] | 2 HCl | ¹H NMR (400 MHz, DMSO) δ 9.53 (d, J = 1.9 Hz, 1H), 9.26-9.20(m, 1H), 9.01 (dd, J = 5.5, 1.3 Hz, 1H), 8.13 (dd, J = 8.1, 5.5 Hz, 1H), 8.07 (d, J = 9.2 Hz, 1H), 7.69 (dd, J = 9.2, 2.7 Hz, 1H), 7.65 (d, J = 1.7 Hz, 1H), 7.47 (d, J = 2.7 Hz, 1H), 7.40 (d, J = 8.0 Hz, 1H), 7.12 | DMSO | >98 | Method C1, E, F3, G2 | | | |
| | | | | | (dd, J = 8.0, 1.9 | | | | | | |
| | | | | | Hz, 1H), 4.69 (t, | | | | | | |
| | | | | | J = 8.0 Hz, 2H), | | | | | | |
| | | | | | 3.91 (s, 3H), 3.24 | | | | | | |
| | | | | | (t, J = 7.9 Hz, 2H). | | | | | | |
| 5 | 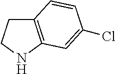 | 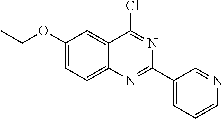 [SMILES: CCOc1ccc2nc(-c3cccnc3)nc(Cl)c2c1] | 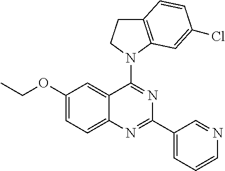 [SMILES: CCOc1ccc2nc(-c3cccnc3)nc(N3CCc4ccc(Cl)cc43)c2c1] | 2 HCl | 1H NMR (400 MHz, DMSO) δ 9.53 (d, J = 1.9 Hz, 1H), 9.30-9.19(m, 1H), 9.01 (dd, J = 5.5, 1.3 Hz, 1H), 8.14 (dd, J = 8.1, 5.5 Hz, 1H), 8.08 (d, J = 9.2 Hz, 1H), 7.72- 7.60 (m, 2H), 7.45 (d, J = 2.6 Hz, | DMSO | >98 | Method C1, E, F3, G2 | | | |
| | | | | | 1H), 7.40 (d, J = | | | | | | |
| | | | | | 8.0 Hz, 1H), 7.12 | | | | | | |
| | | | | | (dd, J = 8.0, 1.9 | | | | | | |
| | | | | | Hz, 1H), 4.67 | | | | | | |
| | | | | | (t, J = 8.0 Hz, 2H), | | | | | | |
| | | | | | 4.17 (q, J = 7.0 | | | | | | |
| | | | | | Hz, 2H), 3.23 (t, J = | | | | | | |
| | | | | | 7.9 Hz, 2H), 1.41 | | | | | | |
| | | | | | (t, J = 7.0 Hz, 3H). | | | | | | |
| 6 | 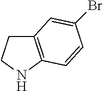 | 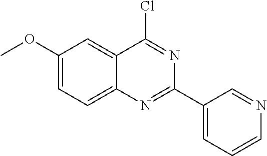 [SMILES: COc1ccc2nc(-c3cccnc3)nc(Cl)c2c1] | 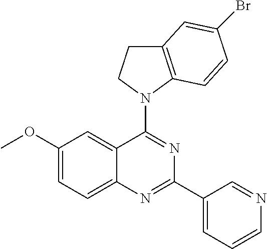 [SMILES: COc1ccc2nc(-c3cccnc3)nc(N3CCc4cc(Br)ccc43)c2c1] | 2 HCl | 1H NMR (400 MHz, DMSO) δ 9.53 (d, J = 2.1 Hz, 1H), 8.72-8.62 (m, 2H), 7.93 (d, J = 9.2 Hz, 1H), 7.60 (dd, J = 9.2, 2.8 Hz, 1H), 7.58-7.51 (m, 2H), 7.47 (d, J = 8.6 Hz, 1H), 7.44- 7.34 (m, 2H), 4.59 (t, J = 8.1 Hz, 2H), 3.88 (s, 3H), 3.29-3.20 (m, 2H). | DMSO | >98 | Method C1, E, F3, G2 | | | |
| 7 | 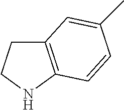 | 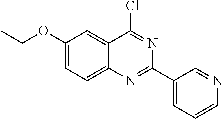 [SMILES: CCOc1ccc2nc(-c3cccnc3)nc(Cl)c2c1] | 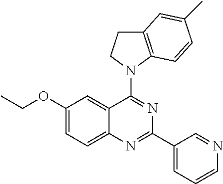 [SMILES: CCOc1ccc2nc(-c3cccnc3)nc(N3CCc4cc(C)ccc43)c2c1] | 2 HCl | 1H NMR (400 MHz, DMSO) δ 9.56 (d, J = 1.8 Hz, 1H), 9.20 (d, J = 8.2 Hz, 1H), 8.99 (dd, J = 5.4, 1.3 Hz, 1H), 8.16 (d, J = 9.1 Hz, 1H), 8.06 (dd, J = 8.1, 5.4 Hz, 1H), 7.73-7.64 (m, 2H), 7.53 (d, J = 2.5 Hz, 1H), 7.23 (s, | DMSO | >98 | Method C1, E, F3, G2 | | | |
| | | | | | 1H), 7.12 (d, J = 8.2 | | | | | | |
| | | | | | Hz, 1H), 4.69 (t, | | | | | | |
| | | | | | J = 7.7 Hz, 2H), | | | | | | |
| | | | | | 4.17 (q, J = 6.9 Hz, | | | | | | |
| | | | | | 2H), 83.21 (t, J = | | | | | | |
| | | | | | 7.6 Hz, 2H), 2.34 | | | | | | |
| | | | | | (s, 3H), 1.45- | | | | | | |
| | | | | | 1.33 (m, 3H). | | | | | | |
| 8 | 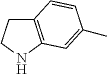 | 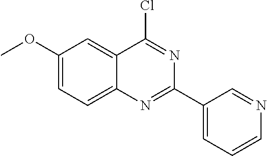 [SMILES: COc1ccc2nc(-c3cccnc3)nc(Cl)c2c1] | 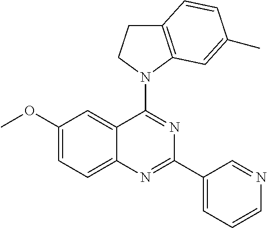 [SMILES: COc1ccc2nc(-c3cccnc3)nc(N3CCc4ccc(C)cc43)c2c1] | 2 HCl | 1H NMR (400 MHz, DMSO) δ 9.55 (d, J = 1.9 Hz, 1H), 9.24 (d, J = 8.2 Hz, 1H), 9.00 (dd, J = 5.4, 1.4 Hz, 1H), 8.11 (dd, J = 8.3, 5.5 Hz, 2H), 7.69 (dd, J = 9.2, 2.7 Hz, 1H), 7.61-7.50 (m, 2H), 7.28 (d, J = 7.6 Hz, 1H), 6.93 (d, J = 7.6 Hz, 1H), 4.68 (t, J = 7.8 Hz, 2H), 3.90 | DMSO | >98 | Method C1, E, F3, G2 | | | |
| | | | | | (s, 3H), 3.19 (dd, J = | | | | | | |
| | | | | | 13.4, 5.7 Hz, 2H), | | | | | | |
| | | | | | 2.35 (d, J = 13.2 | | | | | | |
| | | | | | Hz, 3H). | | | | | | |
| 9 | 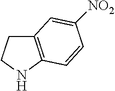 [SMILES: O=[N+]([O-])c1ccc2c(c1)CCN2] | 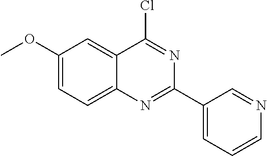 [SMILES: COc1ccc2nc(-c3cccnc3)nc(Cl)c2c1] | 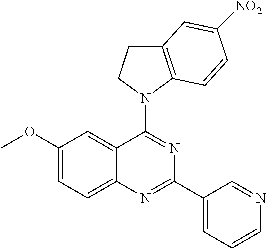 [SMILES: COc1ccc2nc(-c3cccnc3)nc(N3CCc4cc([N+](=O)[O-])ccc43)c2c1] | 2 HCl | 1H NMR (400 MHz, DMSO) δ 9.58 (d, J = 1.6 Hz, 1H), 9.09-9.02 (m, 1H), 8.89 (dd, J = 5.2, 1.4 Hz, 1H), 8.23 (d, J = 2.4 Hz, 1H), 8.16 (dd, J = 8.9, 2.5 Hz, 1H), 8.04 (d, J = 9.2 Hz, 1H), 7.92 (dd, J = 8.1, 5.3 Hz, 1H), 7.70 (dd, J = 9.2, 2.7 Hz, 1H), 7.54 (d, J = 8.9 Hz, 1H), 7.39 (d, J = 2.7 Hz, 1H), | DMSO | >98 | Method C1, E, F3, G2 | | | |
| | | | | | 4.70 (t, J = 8.2 Hz, | | | | | | |
| | | | | | 2H), 3.90 (d, J = | | | | | | |
| | | | | | 5.6 Hz, 3H), 3.36 | | | | | | |
| | | | | | (t, J = 8.1 Hz, 2H). | | | | | | |
| 10 | 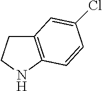 | 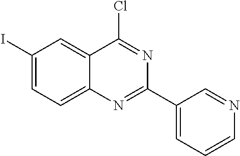 [SMILES: Clc1nc(-c2cccnc2)nc2ccc(I)cc12] | 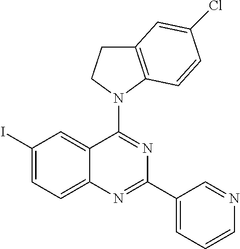 [SMILES: Clc1ccc2c(c1)CCN2c1nc(-c2cccnc2)nc2ccc(I)cc12] | | 1H NMR (400 MHz, DMSO) δ 9.56- 9.47 (m, 1H), 8.70 (dd, J = 4.7, 1.7 Hz, 1H), 8.68- 8.59 (m, 1H), 8.47 (d, J = 1.8 Hz, 1H), 8.15 (dd, J = 8.8, 1.8 Hz, 1H), 7.72 (dd, J = 17.7, 8.7 Hz, 2H), 7.62- 7.51 (m, 1H), 7.43 (d, J = 2.1 Hz, 1H), 7.32 (dd, J = 8.6, 2.3 Hz, 1H), 4.58 (t, J = 8.0 Hz, 2H), | DMSO | >98 | Method D, F3, G2 | | | |
| | | | | | 3.23 (t, J = | | | | | | |
| | | | | | 7.9 Hz, 2H). | | | | | | |
| 11 | 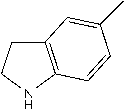 | 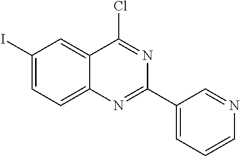 [SMILES: Clc1nc(-c2cccnc2)nc2ccc(I)cc12] | 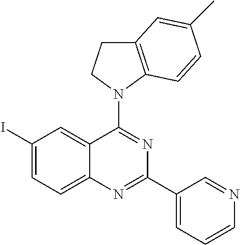 [SMILES: Cc1ccc2c(c1)CCN2c1nc(-c2cccnc2)nc2ccc(I)cc12] | | 1H NMR (400 MHz, DMSO) δ 9.54 (d, J = 1.4 Hz, 1H), 8.76-8.64 (m, 2H), 8.51 (d, J = 1.8 Hz, 1H), 8.14 (dd, J = 8.8, 1.9 Hz, 1H), 7.70 (d, J = 8.8 Hz, 1H), 7.65 (d, J = 8.2 Hz, 1H), 7.59-7.52 (m, 1H), 7.19 (s, 1H), 7.09 (d, J = 8.2 Hz, 1H), 4.57 (t, J = 7.9 Hz, 2H), 3.18 (t, J = 7.8 Hz, 2H), | DMSO | >98 | Method D, F3, G2 | | | |
| | | | | | 2.33 (s, 3H). | | | | | | |
| 12 | 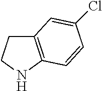 | 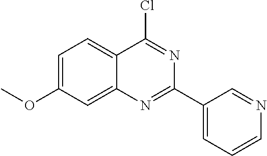 [SMILES: COc1ccc2c(Cl)nc(-c3cccnc3)nc2c1] | 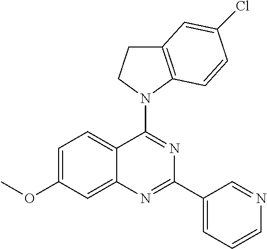 [SMILES: COc1ccc2c(N3CCc4cc(Cl)ccc43)nc(-c3cccnc3)nc2c1] | 2HCl | 1H NMR (400 MHz, DMSO) δ 9.56 (d, J = 1.5 Hz, 1H), 8.95 (d, J = 8.1 Hz, 1H), 8.86 (dd, J = 5.1, 1.5 Hz, 1H), 8.18 (d, J = 9.3 Hz, 1H), 7.91-7.73 (m, 2H), 7.44 (dd, J = 7.2, 2.3 Hz, 2H), 7.29 (ddd, J = 11.9, 9.0, 2.5 Hz, 2H), 4.62 (t, J = 8.0 Hz, 2H), 3.99 (d, J = 7.7 Hz, 3H), 3.24 (t, J = 8.0 Hz, | DMSO | >98 | Method C1, E, F3, G2 | | | |
| | | | | | 2H). | | | | | | |
| 13 | 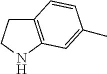 | 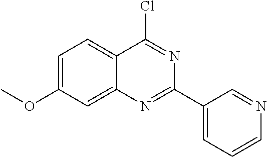 [SMILES: COc1ccc2c(Cl)nc(-c3cccnc3)nc2c1] | 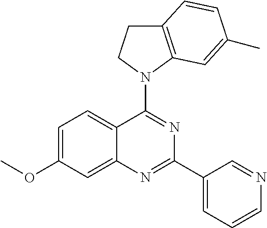 [SMILES: COc1ccc2c(N3CCc4ccc(C)cc43)nc(-c3cccnc3)nc2c1] | | 1H NMR (400 MHz, DMSO) δ 9.62- 9.53 (m, 1H), 8.75- 8.63 (m, 2H), 8.06 (t, J = 8.8 Hz, 1H), 7.66-7.50 (m, 1H), 7.45 (s, 1H), 7.34 (d, J = 2.6 Hz, 1H), 7.27- 7.09 (m, 2H), 6.84 (d, J = 7.5 Hz, 1H), 4.51 (t, J = 8.0 Hz, 2H), 3.97 (d, J = 5.2 Hz, 3H), 3.15 (t, J = 7.8 Hz, | DMSO | >98 | Method C1, E, F3, G2 | | | |
| | | | | | 2H), 2.30 (s, 3H). | | | | | | |
| 14 | 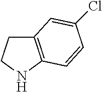 | 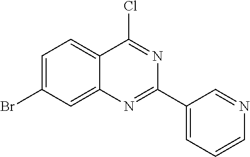 [SMILES: Clc1nc(-c2cccnc2)nc2cc(Br)ccc12] | 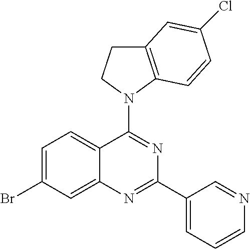 [SMILES: Clc1ccc2c(c1)CCN2c1nc(-c2cccnc2)nc2cc(Br)ccc12] | | 1H NMR (400 MHz, DMSO) δ 9.53 (dd, J = 2.1, 0.7 Hz, 1H), 8.75-8.63 (m, 2H), 8.20- 8.12 (m, 2H), 7.84- 7.78 (m, 1H), 7.72 (dd, J = 9.1, 2.0 Hz, 1H), 7.61- 7.51 (m, 1H), 7.44 (d, J = 2.1 Hz, 1H), 7.33 (dd, J = 8.6, 2.3 Hz, 1H), 4.61 (t, J = 8.0 Hz, 2H), 3.29-3.16 (m, 2H). | DMSO | >98 | Method D, F3, G2 | | | |
| 15 | 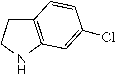 | 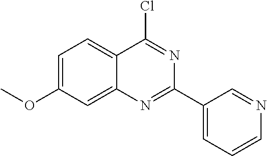 [SMILES: COc1ccc2c(Cl)nc(-c3cccnc3)nc2c1] | 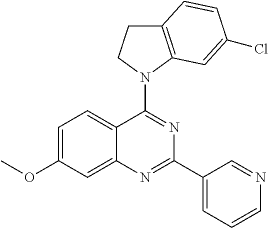 [SMILES: COc1ccc2c(N3CCc4ccc(Cl)cc43)nc(-c3cccnc3)nc2c1] | | 1H NMR (400 MHz, DMSO) δ 9.55 (s, 1H), 8.74-8.63 (m, 2H), 8.12 (d, J = 8.8 Hz, 1H), 7.82- 7.70 (m, 1H), 7.61-7.52 (m, 1H), 7.44-7.31 (m, 2H), 7.21 (d, J = 8.8 Hz, 1H), 7.07 (d, J = 7.7 Hz, 1H), 4.58 (t, J = 7.5 Hz, 2H), 3.99 (s, 3H), 3.21 (t, J = 7.6 Hz, 2H). | DMSO | >98 | Method C1, E, F3, G2 | | | |
| 16 | 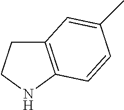 | 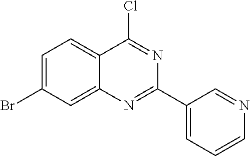 [SMILES: Clc1nc(-c2cccnc2)nc2cc(Br)ccc12] | 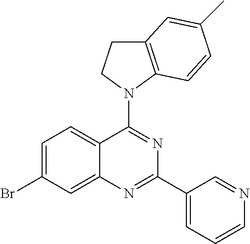 [SMILES: Cc1ccc2c(c1)CCN2c1nc(-c2cccnc2)nc2cc(Br)ccc12] | | 1H NMR (400 MHz, DMSO) δ 9.54 (s, 1H), 8.76-8.62 (m, 2H), 8.21-8.08 (m, 2H), 7.76-7.64 (m, 2H), 7.64- 7.51 (m, 1H), 7.19 (s, 1H), 7.09 (d, J = 8.1 Hz, 1H), 4.57 (t, J = 7.5 Hz, 2H), 3.18 (t, J = 7.6 Hz, 2H), 2.33 (s, 3H). | DMSO | >98 | Method D, F3, G2 | | | |
| 17 | 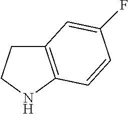 | 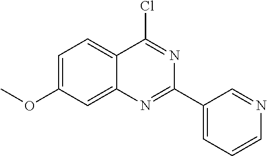 [SMILES: COc1ccc2c(Cl)nc(-c3cccnc3)nc2c1] | 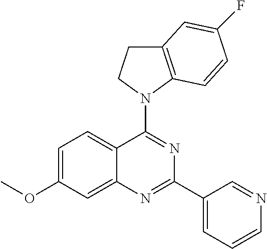 [SMILES: COc1ccc2c(N3CCc4cc(F)ccc43)nc(-c3cccnc3)nc2c1] | | 1H NMR (400 MHz, DMSO) δ 9.55 (d, J = 1.9 Hz, 1H), 8.74-8.63 (m, 2H), 8.12 (d, J = 9.3 Hz, 1H), 7.74 (dd, J = 8.8, 4.8 Hz, 1H), 7.61-7.48 (m, 1H), 7.35 (d, J = 2.6 Hz, 1H), 7.27- 7.16 (m, 2H), 7.16- 7.06 (m, 1H), 4.58 (t, J = 8.1 Hz, 2H), 3.98 (s, 3H), 3.23 (dd, J = 15.2, 7.2 Hz, 2H). | DMSO | >98 | Method C1, E, F3, G2 | | | |
| 18 | 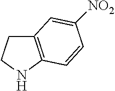 [SMILES: O=[N+]([O-])c1ccc2c(c1)CCN2] | 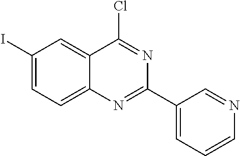 [SMILES: Clc1nc(-c2cccnc2)nc2ccc(I)cc12] | 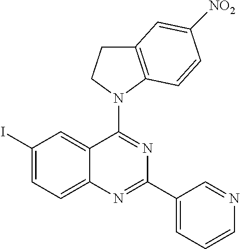 [SMILES: O=[N+]([O-])c1ccc2c(c1)CCN2c1nc(-c2cccnc2)nc2ccc(I)cc12] | | 1H NMR (400 MHz, DMSO) δ 9.56 (d, J = 1.4 Hz, 1H), 8.72 (ddd, J = 8.0, 4.3, 1.7 Hz, 2H), 8.53 (d, J = 1.8 Hz, 1H), 8.30-8.15 (m, 3H), 7.81 (d, J = 8.8 Hz, 1H), 7.72 (d, J = 8.9 Hz, 1H), 7.66-7.53 (m, 1H), 4.69 (t, J = 8.2 Hz, 2H), 3.42- 3.29 (m, 2H). | DMSO | >98 | Method D, F3, G2 | | | |
| 19 | 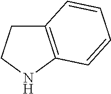 | 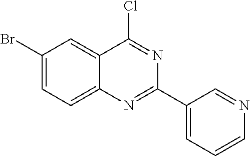 [SMILES: Clc1nc(-c2cccnc2)nc2ccc(Br)cc12] | 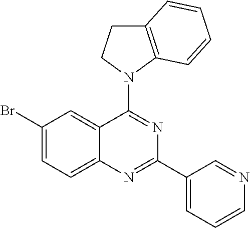 [SMILES: Brc1ccc2nc(-c3cccnc3)nc(N3CCc4ccccc43)c2c1] | | 1H NMR (400 MHz, CDCl3) δ 9.72 (s, 1H), 8.78 (dt, J = 8.0, 1.9 Hz, 1H), 8.81-8.71(m, 1H), 8.24 (dd, J = 1.7, 0.7 Hz, 1H), 7.94- 7.83 (m, 2H), 7.47-7.38 (m, 2H), 7.33 (d, J = 6.9 Hz, 1H), 7.21 (t, J = 7.3 Hz, 1H), 7.05 (dt, J = 7.4, 3.7 Hz, 1H), 4.53 (t, J = 8.0 Hz, 2H), | CDCl3 | >98 | Method D, F3, G2 | | | |
| | | | | | 3.28 (t, J = 7.9 Hz, | | | | | | |
| | | | | | 2H). | | | | | | |
| 20 | 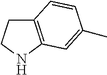 | 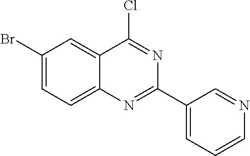 [SMILES: Clc1nc(-c2cccnc2)nc2ccc(Br)cc12] | 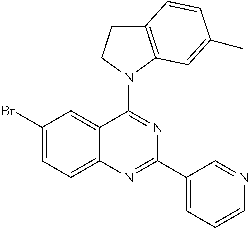 [SMILES: Cc1ccc2c(c1)N(c1nc(-c3cccnc3)nc3ccc(Br)cc13)CC2] | | 1H NMR (400 MHz, CDCl3) δ 9.72 (dd, J = 2.1, 0.8 Hz, 1H), 8.83-8.74 (m, 1H), 8.70 (dd, J = 4.8, 1.7 Hz, 1H), 8.25 (dd, J = 1.7, 0.8 Hz, 1H), 7.93-7.84 (m, 2H), 7.46-7.36 (m, 1H), 7.30-7.25 (m, 1H), 7.20 (d, J = 7.6 Hz, 1H), 6.86 (d, J = 7.5 Hz, 1H), 4.51 (t, J = | CDCl3 | >98 | Method D, F3, G2 | | | |
| | | | | | 7.9 Hz, 2H), 3.22 (t, | | | | | | |
| | | | | | J = 7.9 Hz, 2H), | | | | | | |
| | | | | | 2.35 (s, 3H). | | | | | | |
| 21 | 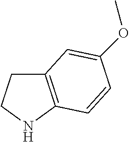 | 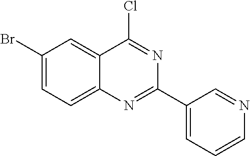 [SMILES: Clc1nc(-c2cccnc2)nc2ccc(Br)cc12] | 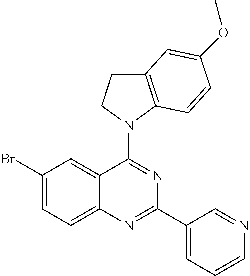 [SMILES: COc1ccc2c(c1)CCN2c1nc(-c2cccnc2)nc2ccc(Br)cc12] | | 1H NMR (400 MHz, CDCl3) δ 9.70 (d, J = 1.5 Hz, 1H), 8.76 (dd, J = 8.0, 2.0 Hz, 1H), 8.69 (dd, J = 4.8, 1.7 Hz, 1H), 8.23 (s, 1H), 7.85 (s, 2H), 7.58 (d, J = 8.8 Hz, 1H), 7.40 (dd, J = 8.0, 4.8 Hz, 1H), 6.89 (d, J = 2.5 Hz, 1H), 6.79 (dd, J = 8.8, 2.6 Hz, 1H), 4.53 (t, J = 7.9 Hz, 2H), 3.84 (s, 3H), 3.24 (t, J = 7.8 Hz, 2H). | CDCl3 | >98 | Method D, F3, G2 | | | |
| 22 | 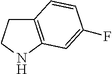 | 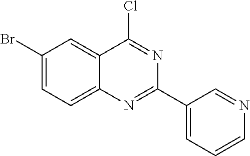 [SMILES: Clc1nc(-c2cccnc2)nc2ccc(Br)cc12] | 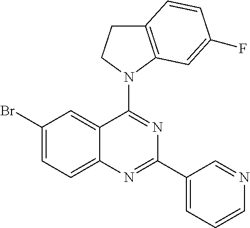 [SMILES: Fc1ccc2c(c1)N(c1nc(-c3cccnc3)nc3ccc(Br)cc13)CC2] | | 1H NMR (400 MHz, CDCl3) δ 9.76- 9.68 (m, 1H), 8.81-8.68 (m, 2H), 8.21 (s, 1H), 7.96- 7.86 (m, 2H), 7.42 (dd, J = 8.0, 4.8 Hz, 1H), 7.25- 7.11 (m, 2H), 6.73 (td, J = 8.6, 2.3 Hz, 1H), 4.56 (t, J = 8.0 Hz, 2H), 3.23 (t, J = 7.9 Hz, 2H). | CDCl3 | >98 | Method D, F3, G2 | | | |
| 23 | 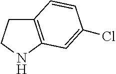 | 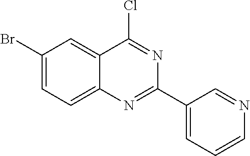 [SMILES: Clc1nc(-c2cccnc2)nc2ccc(Br)cc12] | 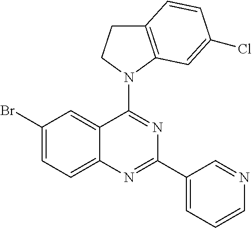 [SMILES: Clc1ccc2c(c1)N(c1nc(-c3cccnc3)nc3ccc(Br)cc13)CC2] | | 1H NMR (400 MHz, CDCl3) δ 9.72 (dd, J = 2.2, 0.8 Hz, 1H), 8.82-8.67 (m, 2H), 8.21 (dd, J = 1.7, 0.9 Hz, 1H), 7.97-7.87 (m, 2H), 7.49 (d, J = 1.8 Hz, 1H), 7.47- 7.39 (m, 1H), 7.22 (d, J = 8.0 Hz, 1H), 7.01 (dd, J = 7.9, 1.9 Hz, 1H), 4.56 (t, J = 8.0 Hz, 2H), 3.25 (t, | CDCl3 | >98 | Method D, F3, G2 | | | |
| | | | | | J = 8.0 Hz, 2H). | | | | | | |
| 24 | 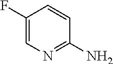 | 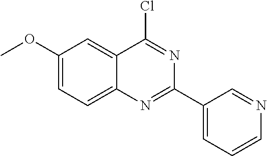 [SMILES: COc1ccc2nc(-c3cccnc3)nc(Cl)c2c1] | 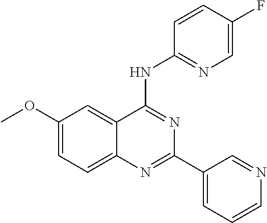 [SMILES: COc1ccc2nc(-c3cccnc3)nc(Nc3ccc(F)cn3)c2c1] | HCl | ¹H NMR (400 MHz, DMSO) δ 11.01 (s, 1H), 9.55 (d, J = 1.7 Hz, 1H), 9.20 (d, J = 8.1 Hz, 1H), 9.04-8.93 (m, 1H), 8.53 (d, J = 3.0 Hz, 1H), 8.41 (dd, J = 9.1, 4.1 Hz, 1H), 8.23 (d, J = 2.5 Hz, 1H), 8.09 (dd, J = 8.0, 5.6 Hz, 1H), 8.00 (d, J = 9.1 Hz, 1H), 7.95-7.89 (m, | DMSO | >98 | Method C2, E(NaOH_{(aq.)} instead of NaOH and Δ), F2, H1 | | | |
| | | | | | 1H), 7.63 (dd, J = | | | | | | |
| | | | | | 9.1, 2.6 Hz, | | | | | | |
| | | | | | 1H), 3.99 (s, 3H). | | | | | | |
| 25 | 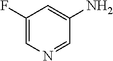 | 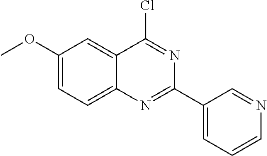 [SMILES: COc1ccc2nc(-c3cccnc3)nc(Cl)c2c1] | 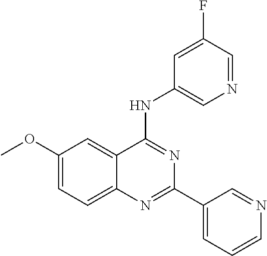 [SMILES: COc1ccc2nc(-c3cccnc3)nc(Nc3cncc(F)c3)c2c1] | 2 HCl | H NMR (400 MHz, DMSO) δ 10.88 (s, 1H), 9.50 (d, J = 1.9 Hz, 1H), 9.17- 9.12 (m, 1H), 9.12-9.08 (m, 1H), 8.97 (dd, J = 5.5, 1.4 Hz, 1H), 8.52- 8.43 (m, 2H), 8.30 (d, J = 2.6 Hz, 1H), 8.09 (dd, J = 8.1, 5.5 Hz, 1H), 7.98 (d, J = 9.1 Hz, 1H), 7.65 (dd, J = 9.1, 2.7 Hz, 1H), 4.03 (s, 3H). | DMSO | >98 | Method C2, E(NaOH_{(aq.)} instead of NaOH and Δ), F2, H1 | | | |
| 26 | 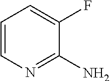 | 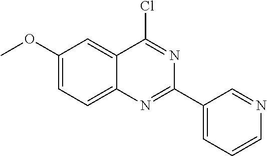 [SMILES: COc1ccc2nc(-c3cccnc3)nc(Cl)c2c1] | 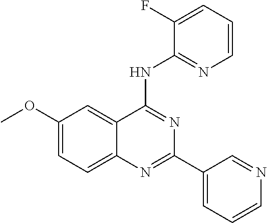 [SMILES: COc1ccc2nc(-c3cccnc3)nc(Nc3ncccc3F)c2c1] | 2 HCl | 1H NMR (400 MHz, DMSO) δ 10.92 (s, 1H), 9.33 (d, J = 1.8 Hz, 1H), 8.95- 8.85 (m, 2H), 8.46-8.39 (m, 1H), 8.10 (d, J = 2.7 Hz, 1H), 8.02-7.90 (m, 3H), 7.64 (dd, J = 9.1, 2.7 Hz, 1H), 7.57-7.50 (m, 1H), 3.99 (s, 3H). | DMSO | >98 | Method C2, E(NaOH_{(aq.)} instead of NaOH and Δ), F2, H1 | | | |
| 27 | 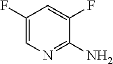 | 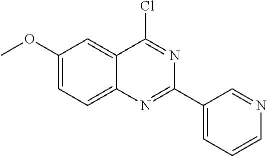 [SMILES: COc1ccc2nc(-c3cccnc3)nc(Cl)c2c1] | 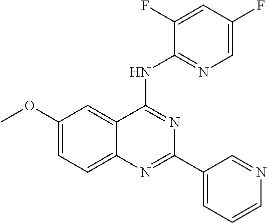 [SMILES: COc1ccc2nc(-c3cccnc3)nc(Nc3ncc(F)cc3F)c2c1] | 2 HCl | 1H NMR (400 MHz, DMSO) δ 11.04 (s, 1H), 9.35 (d, J = 1.9 Hz, 1H), 9.03- 8.97 (m, 1H), 8.94 (dd, J = 5.5, 1.3 Hz, 1H), 8.54 (d, J = 2.6 Hz, 1H), 8.24-8.17 (m, 1H), 8.13 (d, J = 2.7 Hz, 1H), 8.04 (dd, J = 8.1, 5.5 Hz, 1H), 7.99 (d, J = 9.1 Hz, 1H), 7.66 (dd, J = 9.1, 2.7 | DMSO | >98 | Method C2, E(NaOH_{(aq.)} instead of NaOH and Δ), F2, H1 | | | |
| | | | | | Hz, 1H), 3.99 (s, | | | | | | |
| | | | | | 3H). | | | | | | |
| 28 | 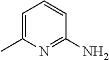 | 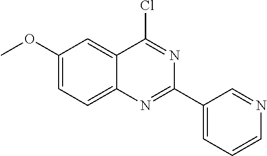 [SMILES: COc1ccc2nc(-c3cccnc3)nc(Cl)c2c1] | 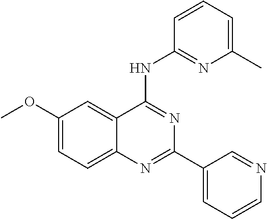 | 2 HCl | 1H NMR (400 MHz, DMSO) δ 11.65 (s, 1H), 9.57 (d, J = 1.8 Hz, 1H), 9.21- 9.10 (m, 1H), 8.97 (dd, J = 5.4, 1.4 Hz, 1H), 8.44 (d, J = 8.4 Hz, 1H), 8.38 (d, J = 2.2 Hz, 1H), 8.16 (t, J = 8.0 Hz, 1H), 8.04 (dd, J = 8.1, 5.4 Hz, 1H), 8.00 (d, J = 9.1 Hz, 1H), | DMSO | >98 | Method C2, E(NaOH_{(aq.)} instead of NaOH and Δ), F2, H1 | | | |
| | | | | | 7.66 (dd, J = 9.1, | | | | | | |
| | | | | | 2.7 Hz, 1H), 7.33 | | | | | | |
| | | | | | (d, J = 7.5 Hz, 1H), | | | | | | |
| | | | | | 4.02 (s, 3H), 2.66 | | | | | | |
| | | | | | (s, 3H). | | | | | | |
| 29 | 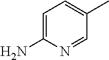 | 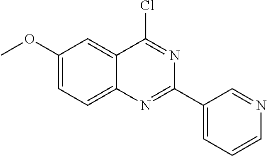 [SMILES: COc1ccc2nc(-c3cccnc3)nc(Cl)c2c1] | 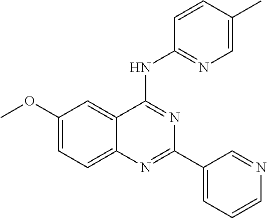 | 2 HCl | 1H NMR (400 MHz, DMSO) δ 11.48 (s, 1H), 9.57 (d, J = 1.8 Hz, 1H), 9.19- 9.11 (m, 1H), 8.95 (dd, J = 5.4, 1.4 Hz, 1H), 8.46- 8.42 (m, 1H), 8.41 (d, J = 8.6 Hz, 1H), 8.34 (d, J = 2.5 Hz, 1H), 8.07- 7.95 (m, 3H), 7.66 (dd, J = 9.1, 2.7 Hz, 1H), 4.01 (s, 3H), 2.40 (s, 3H). | DMSO | >98 | Method C2, E(NaOH_{(aq.)} instead of NaOH and Δ), F2, H1 | | | |
| 30 | 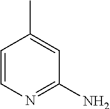 | 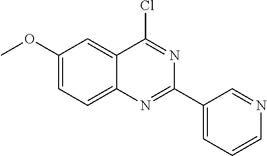 [SMILES: COc1ccc2nc(-c3cccnc3)nc(Cl)c2c1] | 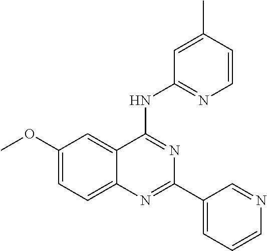 [SMILES: COc1ccc2nc(-c3cccnc3)nc(Nc3cc(C)ccn3)c2c1] | 2 HCl | 1H NMR (400 MHz, DMSO) δ 11.79 (s, 1H), 9.57 (d, J = 1.7 Hz, 1H), 9.15 (d, J = 8.2 Hz, 1H), 8.96 (dd, J = 5.4, 1.3 Hz, 1H), 8.49 (d, J = 5.7 Hz, 1H), 8.44-8.36 (m, 2H), 8.04 (dd, J = 8.1, 5.4 Hz, 1H), 7.99 (d, J = 9.1 Hz, 1H), 7.67 (dd, J = 9.1, 2.7 Hz, 1H), 7.34 (d, J = 4.8 Hz, 1H), 4.03 (s, 3H), 2.58 (s, 3H). | DMSO | >98 | Method C2, E(NaOH_{(aq.)} instead of NaOH and Δ), F2, H1 | | | |
| 31 | 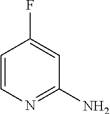 | 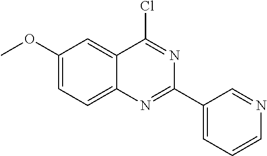 [SMILES: COc1ccc2nc(-c3cccnc3)nc(Cl)c2c1] | 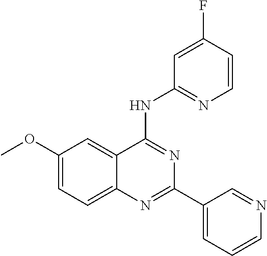 [SMILES: COc1ccc2nc(-c3cccnc3)nc(Nc3cc(F)ccn3)c2c1] | 2 HCl | 1H NMR (400 MHz, DMSO) δ 11.17 (s, 1H), 9.54 (d, J = 1.9 Hz, 1H), 9.23- 9.17 (m, 1H), 9.00 (dd, J = 5.5, 1.3 Hz, 1H), 8.54 (dd, J = 9.1, 5.8 Hz, 1H), 8.34 (dd, J = 11.9, 2.3 Hz, 1H), 8.25 (d, J = 2.6 Hz, 1H), 8.13 (dd, J = 8.1, 5.5 Hz, 1H), 7.97 (d, J = 9.1 Hz, 1H), 7.63 (dd, J = 9.1, 2.7 Hz, 1H), 7.23 (ddd, J = | DMSO | >98 | Method C2, E(NaOH_{(aq.)} instead of NaOH and Δ), F2, H1 | | | |
| | | | | | 8.2, 5.8, 2.4 Hz, | | | | | | |
| | | | | | 1H), 4.00 (s, 3H). | | | | | | |
| 32 | 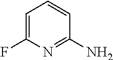 | 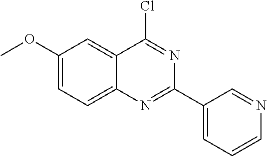 [SMILES: COc1ccc2nc(-c3cccnc3)nc(Cl)c2c1] | 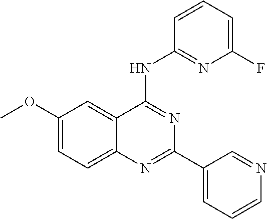 [SMILES: COc1ccc2nc(-c3cccnc3)nc(Nc3cccc(F)n3)c2c1] | HCl | 1H NMR (400 MHz, DMSO) δ 10.84 (s, 1H), 9.58 (d, J = 1.7 Hz, 1H), 9.17- 9.09 (m, 1H), 8.93 (dd, J = 5.3, 1.4 Hz, 1H), 8.44 (dd, J = 8.0, 2.2 Hz, 1H), 8.20 (d, J = 2.7 Hz, 1H), 8.14 (dd, J = 16.8, 8.1 Hz, 1H), 8.00 (dd, J = 8.0, 5.3 Hz, 1H), 7.93 (d, J = 9.1 | DMSO | >98 | Method C2, E(NaOH_{(aq.)} instead of NaOH and Δ), F2, H1 | | | |
| | | | | | Hz, 1H), 7.61 (dd, | | | | | | |
| | | | | | J = 9.1, 2.7 Hz, | | | | | | |
| | | | | | 1H), 6.98 (dd, J = | | | | | | |
| | | | | | 7.9, 2.5 Hz, 1H), | | | | | | |
| | | | | | 3.99 (s, 3H). | | | | | | |
| 33 | 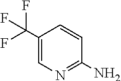 [SMILES: Nc1ccc(C(F)(F)F)cn1] | 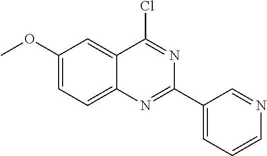 [SMILES: COc1ccc2nc(-c3cccnc3)nc(Cl)c2c1] | 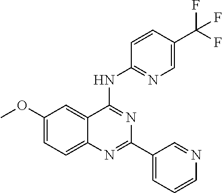 [SMILES: COc1ccc2nc(-c3cccnc3)nc(Nc3ccc(C(F)(F)F)cn3)c2c1] | 2 HCl | 1H NMR (400 MHz, DMSO) δ 11.18 (s, 1H), 9.60 (d, J = 1.8 Hz, 1H), 9.27- 9.21 (m, 1H), 8.98 (dd, J = 5.5, 1.2 Hz, 1H), 8.90- 8.85 (m, 1H), 8.69 (d, J = 8.9 Hz, 1H), 8.32 (dd, J = 9.0, 2.4 Hz, 1H), 8.23 (d, J = 2.6 Hz, 1H), 8.10 (dd, J = 8.1, | DMSO | >98 | Method C2, E(NaOH_{(aq.)} instead of NaOH and Δ), F2, H1 | | | |
| | | | | | 5.5 Hz, 1H), 7.96 | | | | | | |
| | | | | | (d, J = 9.1 Hz, 1H), | | | | | | |
| | | | | | 7.64 (dd, J = 9.1, | | | | | | |
| | | | | | 2.7 Hz, 1H), | | | | | | |
| | | | | | 4.00 (s, 3H). | | | | | | |
| 34 | 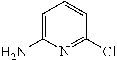 | 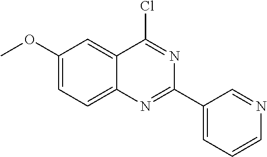 [SMILES: COc1ccc2nc(-c3cccnc3)nc(Cl)c2c1] | 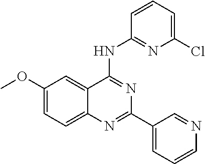 [SMILES: COc1ccc2nc(-c3cccnc3)nc(Nc3cccc(Cl)n3)c2c1] | 2 HCl | 1H NMR (400 MHz, DMSO) δ 10.95 (s, 1H), 9.58 (d, J = 1.7 Hz, 1H), 9.13 (d, J = 8.1 Hz, 1H), 8.92 (d, J = 5.2 Hz, 1H), 8.48 (d, J = 8.2 Hz, 1H), 8.22 (d, J = 2.7 Hz, 1H), 8.06-7.97 (m, 2H), 7.95 (d, | DMSO | >98 | Method C2, E(NaOH_{(aq.)} instead of NaOH and Δ), F2, G1 | | | |
| | | | | | J = 9.1 Hz, 1H), | | | | | | |
| | | | | | 7.63 (dd, J = 9.1, | | | | | | |
| | | | | | 2.7 Hz, 1H), 7.33 | | | | | | |
| | | | | | (d, J = 7.2 Hz, | | | | | | |
| | | | | | 1H), 4.00 (s, 3H). | | | | | | |
| 35 | 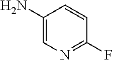 | 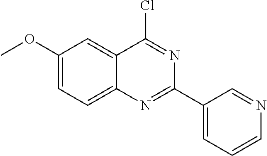 [SMILES: COc1ccc2nc(-c3cccnc3)nc(Cl)c2c1] | 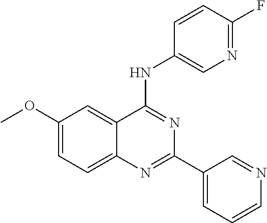 [SMILES: COc1ccc2nc(-c3cccnc3)nc(Nc3ccc(F)nc3)c2c1] | HCl | 1H NMR (400 MHz, DMSO) δ 10.54 (s, 1H), 9.47 (s, 1H), 8.98 (d, J = 8.1 Hz, 1H), 8.88 (d, J = 4.8 Hz, 1H), 8.73 (d, J = 1.4 Hz, 1H), 8.49 (ddd, J = 8.8, 7.4, 2.8 Hz, 1H), 8.14 (d, J = 2.6 Hz, 1H), 7.99- 7.89 (m, 2H), 7.63 (dd, J = 9.1, 2.7 Hz, 1H), 7.35 | DMSO | >98 | Method C2, E(NaOH_{(aq.)} instead of NaOH and Δ), F2, H1 | | | |
| | | | | | (dd, J = 8.8, 3.2 | | | | | | |
| | | | | | Hz, 1H), 4.01 (s, | | | | | | |
| | | | | | 3H). | | | | | | |
| 36 | 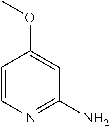 | 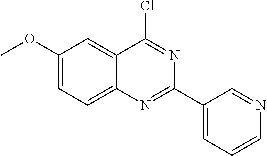 [SMILES: COc1ccc2nc(-c3cccnc3)nc(Cl)c2c1] | 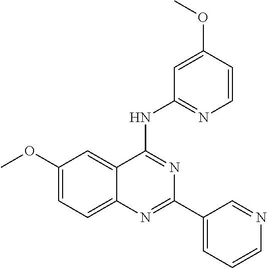 [SMILES: COc1ccnc(Nc2nc(-c3cccnc3)nc3ccc(OC)cc23)c1] | 2 HCl | 1H NMR (400 MHz, DMSO) δ 12.35 (s, 1H), 9.58 (d, J = 1.5 Hz, 1H), 9.14 (d, J = 8.2 Hz, 1H), 8.96 (d, J = 4.2 Hz, 1H), 8.54 (d, J = 7.0 Hz, 1H), 8.49 (d, J = 1.9 Hz, 1H), 8.35 (d, J = 2.1 Hz, 1H), 8.03 (dd, J = 8.1, 5.4 Hz, 1H), 7.96 (d, J = 9.1 Hz, 1H), 7.66 (dd, J = 9.1, 2.7 Hz, 1H), 7.19 (dd, J = 7.0, 2.6 | DMSO | >98 | Method C2, E(NaOH_{(aq.)} instead of NaOH and Δ), F2, H1 | | | |
| | | | | | Hz, 1H), 4.11 (s, | | | | | | |
| | | | | | 3H), 4.02 (s, 3H). | | | | | | |
| 37 | 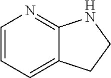 | 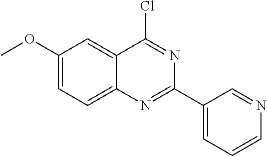 [SMILES: COc1ccc2nc(-c3cccnc3)nc(Cl)c2c1] | 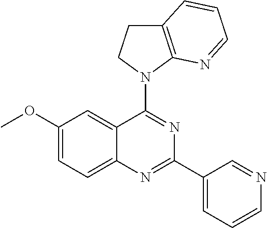 [SMILES: COc1ccc2nc(-c3cccnc3)nc(N3CCc4cccnc43)c2c1] | 2 HCl | 1H NMR (400 MHz, DMSO) δ 9.69 (s, 1H), 9.29-9.22 (m, 1H), 8.95 (d, J = 4.2 Hz, 1H), 8.07- 7.95 (m, 3H), 7.80-7.73 (m, 1H), 7.67 (dd, J = 9.2, 2.8 Hz, 1H), 7.49 (d, J = 2.8 Hz, 1H), 6.99 (dd, J = 7.2, 5.2 Hz, 1H), 4.54 (t, J = 8.1 Hz, 2H), 3.78 (s, 3H), 3.30 (t, J = | DMSO | >98 | Method C2, E(NaOH_{(aq.)} instead of NaOH and Δ), F2, H1 | | | |
| | | | | | 8.0 Hz, 2H). | | | | | | |
| 38 | 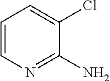 | 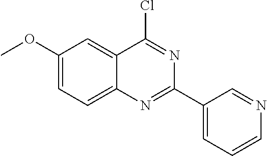 [SMILES: COc1ccc2nc(-c3cccnc3)nc(Cl)c2c1] | 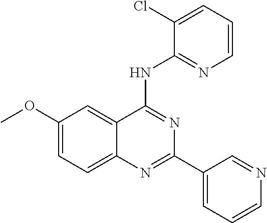 [SMILES: COc1ccc2nc(-c3cccnc3)nc(Nc3ncccc3Cl)c2c1] | 2 HCl | 1H NMR (400 MHz, DMSO) δ 11.10 (s, 1H), 9.29 (d, J = 1.8 Hz, 1H), 8.99- 8.90 (m, 2H), 8.58 (dd, J = 4.7, 1.6 Hz, 1H), 8.21 (dd, J = 8.0, 1.6 Hz, 1H), 8.15-8.09 (m, 1H), 8.05-7.97 (m, 2H), 7.67 (dd, J = 9.1, 2.6 Hz, 1H), 7.54 (dd, J = 8.0, 4.7 Hz, 1H), 3.99 (s, 3H). | DMSO | >98 | Method C2, E(NaOH_{(aq.)} instead of NaOH and Δ), F2, H1 | | | |
| 39 | 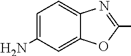 | 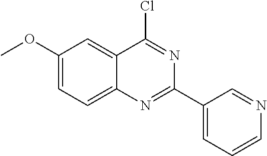 [SMILES: COc1ccc2nc(-c3cccnc3)nc(Cl)c2c1] | 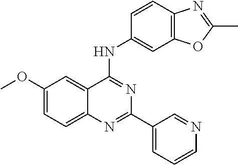 | | 1H NMR (400 MHz, CDCl3) δ 9.71 (dd, J = 2.2, 0.8 Hz, 1H), 8.78-8.74 (m, 1H), 8.69 (dd, J = 4.8, 1.7 Hz, 1H), 8.43 (d, J = 1.9 Hz, 1H), 7.95 (d, J = 9.1 Hz, 1H), 7.67 (d, J = 8.5 Hz, 1H), 7.55- | CDCl3 | >98 | Method C2, E(NaOH_{(aq.)} instead of NaOH and Δ), F2, H1 | | | |
| | | | | | 7.47 (m, 2H), 7.46- | | | | | | |
| | | | | | 7.39 (m, 2H), 7.15 | | | | | | |
| | | | | | (d, J = 2.6 Hz, | | | | | | |
| | | | | | 1H), 3.99 (s, | | | | | | |
| | | | | | 3H), 2.68 (s, 3H). | | | | | | |
| 40 | 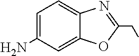 | 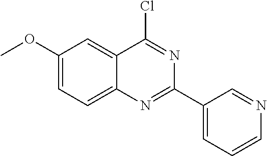 [SMILES: COc1ccc2nc(-c3cccnc3)nc(Cl)c2c1] | 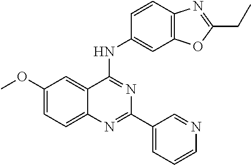 [SMILES: CCc1nc2ccc(Nc3nc(-c4cccnc4)nc4ccc(OC)cc34)cc2o1] | | 1H NMR (400 MHz, CDCl3) δ 9.71 (dd, J = 2.2, 0.8 Hz, 1H), 8.80-8.74 (m, 1H), 8.69 (dd, J = 4.8, 1.7 Hz, 1H), 8.45 (d, J = 1.9 Hz, 1H), 7.96 (d, J = 9.1 Hz, 1H), 7.70 (d, J = 8.4 Hz, 1H), 7.51 | CDCl3 | >98 | Method C2, E(NaOH_{(aq.)} instead of NaOH and Δ), F2, H1 | | | |
| | | | | | (dd, J = 9.2, 2.6 Hz, | | | | | | |
| | | | | | 1H), 7.47-7.39 | | | | | | |
| | | | | | (m, 3H), 7.15 (d, J = | | | | | | |
| | | | | | 2.6 Hz, 1H), 4.01 | | | | | | |
| | | | | | (s, 3H), 3.01 (q, J = | | | | | | |
| | | | | | 7.6 Hz, 2H), 1.50 | | | | | | |
| | | | | | (t, J = 7.6 Hz, | | | | | | |
| | | | | | 3H). | | | | | | |
| 41 | 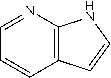 | 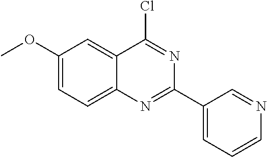 [SMILES: COc1ccc2nc(-c3cccnc3)nc(Cl)c2c1] | 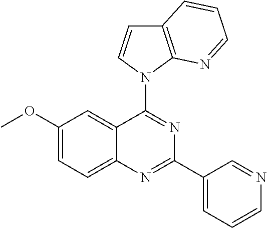 [SMILES: COc1ccc2nc(-c3cccnc3)nc(-n3ccc4cccnc43)c2c1] | | 1H NMR (400 MHz, DMSO) δ 9.66 (s, 1H), 8.87-8.81 (m, 1H), 8.76 (d, J = 3.7 Hz, 1H), 8.31 (dd, J = 4.6, 1.6 Hz, 1H), 8.26 (d, J = 3.7 Hz, 1H), 8.22 (dd, J = 7.8, 1.6 Hz, 1H), 8.18 (d, J = 9.2 Hz, 1H), 7.80 (dd, J = 9.2, 2.8 Hz, 1H), 7.64 (dd, J = 7.6, 5.0 Hz, 1H), 7.33 (dd, | DMSO | >98 | Method C2, E(NaOH_{(aq.)} instead of NaOH and Δ), F2, H1 | | | |
| | | | | | J = 7.8, 4.7 Hz, | | | | | | |
| | | | | | 1H), 7.29 (d, J = | | | | | | |
| | | | | | 2.8 Hz, 1H), 6.98 | | | | | | |
| | | | | | (d, J = 3.8 Hz, | | | | | | |
| | | | | | 1H), 3.73 (s, 3H). | | | | | | |
| 42 | 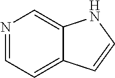 | 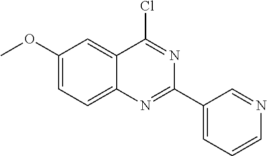 [SMILES: COc1ccc2nc(-c3cccnc3)nc(Cl)c2c1] | 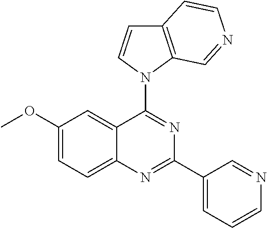 [SMILES: COc1ccc2nc(-c3cccnc3)nc(-n3ccc4ccncc43)c2c1] | 2 HCl | 1H NMR (400 MHz, DMSO) δ 9.73- 9.67 (m, 2H), 9.12- 9.04 (m, 2H), 8.90 (dd, J = 5.1, 1.5 Hz, 1H), 8.61 (d, J = 6.5 Hz, 1H), 8.42 (d, J = 6.3 Hz, 1H), 8.28 (d, J = 9.2 Hz, 1H), 7.93-7.85 (m, 2H), 7.44 (d, J = 2.8 Hz, 1H), 7.42 (d, J = 2.7 Hz, 1H), 3.95 (s, 3H). | DMSO | >98 | Method C2, E(NaOH_{(aq.)} instead of NaOH and Δ), F2, H1 | | | |
| 43 | 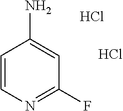 | 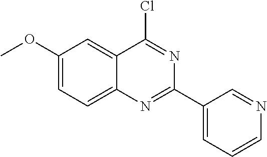 [SMILES: COc1ccc2nc(-c3cccnc3)nc(Cl)c2c1] | 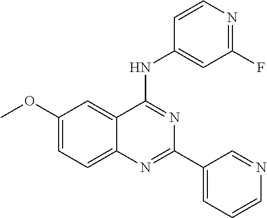 [SMILES: COc1ccc2nc(-c3cccnc3)nc(Nc3ccnc(F)c3)c2c1] | HCl | 1H NMR (400 MHz, DMSO) δ 10.46 (s, 1H), 9.57 (s, 1H), 9.01-8.95 (m, 1H), 8.86 (d, J = 4.4 Hz, 1H), 8.25 (d, J = 5.7 Hz, 1H), 8.09 (d, J = 2.7 Hz, 1H), 7.99-7.93 (m, 2H), 7.91- 7.86 (m, 2H), 7.66 (dd, J = 9.1, 2.7 Hz, 1H), 4.02 (s, 3H). | DMSO | >98 | Method C2, E(NaOH_{(aq.)} instead of NaOH and Δ), F2, H1 | | | |
| 44 | 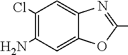 | 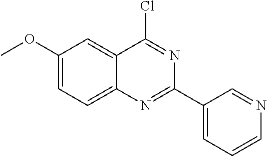 [SMILES: COc1ccc2nc(-c3cccnc3)nc(Cl)c2c1] | 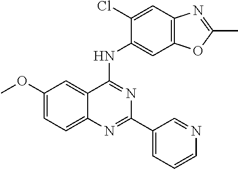 | | 1H NMR (400 MHz, DMSO) δ 10.02 (s, 1H), 9.23 (dd, J = 2.1, 0.8 Hz, 1H), 8.58 (dd, J = 4.7, 1.7 Hz, 1H), 8.45-8.41 (m, 1H), 8.07 (s, 1H), 8.02 (s, 1H), 7.99 (d, J = 2.7 Hz, 1H), 7.85 (d, J = 9.1 Hz, | DMSO | >98 | Method C2, E(NaOH_{(aq.)} instead of NaOH and Δ), F2, H1 | | | |
| | | | | | 1H), 7.56 (dd, J = | | | | | | |
| | | | | | 9.1, 2.7 Hz, 1H), | | | | | | |
| | | | | | 7.43 (ddd, J = | | | | | | |
| | | | | | 8.0, 4.8, 0.8 Hz, | | | | | | |
| | | | | | 1H), 3.97 (s, 3H), | | | | | | |
| | | | | | 2.68 (s, 3H). | | | | | | |
| 45 | 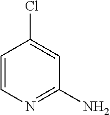 | 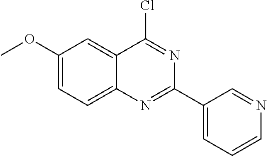 [SMILES: COc1ccc2nc(-c3cccnc3)nc(Cl)c2c1] | 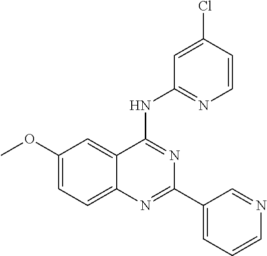 [SMILES: COc1ccc2nc(-c3cccnc3)nc(Nc3cc(Cl)ccn3)c2c1] | | 1H NMR (400 MHz, DMSO) δ 10.82 (s, 1H), 9.55 (dd, J = 2.1, 0.8 Hz, 1H), 8.76 (d, J = 1.7 Hz, 1H), 8.71- 8.66 (m, 2H), 8.46 (d, J = 5.4 Hz, 1H), 8.19 (d, J = 2.7 Hz, 1H), 7.89 (d, J = 9.1 Hz, 1H), 7.60-7.55 (m, 2H), 7.34 (dd, J = 5.4, 1.9 Hz, 1H), 3.98 (s, 3H). | DMSO | >98 | Method C2, E(NaOH_{(aq.)} instead of NaOH and Δ), F2, G3(Cs2CO3 instead of K2CO3, dioxane instead of DMF) | | | |
| 46 | 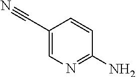 | 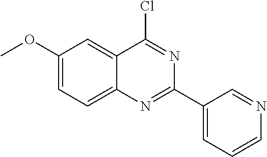 [SMILES: COc1ccc2nc(-c3cccnc3)nc(Cl)c2c1] | 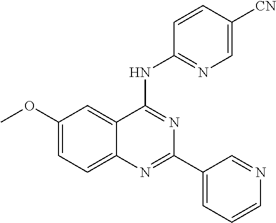 [SMILES: COc1ccc2nc(-c3cccnc3)nc(Nc3ccc(C#N)cn3)c2c1] | HCl | 1H NMR (400 MHz, DMSO) δ 11.16 (s, 1H), 9.58 (s, 1H), 9.07 (d, J = 8.0 Hz, 1H), 8.94 (dd, J = 2.3, 0.7 Hz, 1H), 8.90 (s, 1H), 8.67-8.62 (m, 1H), 8.38 (dd, J = 8.8, 2.3 Hz, 1H), 8.19 (d, J = 2.7 Hz, 1H), 7.97-7.91 (m, 2H), 7.62 (dd, J = 9.1, 2.7 Hz, 1H), 3.99 (s, 3H). | DMSO | >98 | Method C2, E(NaOH_{(aq.)} instead of NaOH and Δ), F2, H1 | | | |
| 47 | 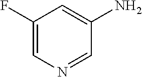 | 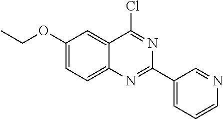 [SMILES: CCOc1ccc2nc(-c3cccnc3)nc(Cl)c2c1] | 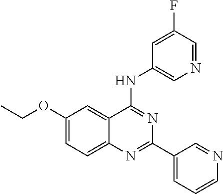 [SMILES: CCOc1ccc2nc(-c3cccnc3)nc(Nc3cncc(F)c3)c2c1] | 2 HCl | 1H NMR (400 MHz, DMSO) δ 10.63 (s, 1H), 9.51 (s, 1H), 9.16-8.99 (m, 2H), 8.93 (s, 1H), 8.51-8.37 (m, 2H), 8.19 (s, 1H), 8.08-7.91 (m, 2H), 7.65 (dd, J = 9.1, 2.6 Hz, 1H), 4.30 (q, J = 6.9 Hz, 2H), 1.46 (t, J = 6.9 Hz, 3H). | DMSO | >98 | Method C1, E, F3, G1 | | | |
| 48 | 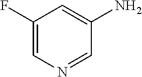 | 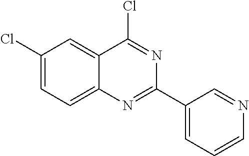 [SMILES: Clc1ccc2nc(-c3cccnc3)nc(Cl)c2c1] | 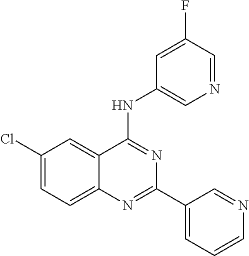 [SMILES: Fc1cncc(Nc2nc(-c3cccnc3)nc3ccc(Cl)cc23)c1] | 2 HCl | 1H NMR (400 MHz, DMSO) δ 10.72 (s, 1H), 9.51 (d, J = 1.8 Hz, 1H), 9.13- 9.08 (m, 1H), 9.06- 9.02 (m, 1H), 8.97 (dd, J = 5.4, 1.4 Hz, 1H), 8.94- 8.90 (m, 1H), 8.46 (d, J = 2.6 Hz, 1H), 8.44-8.39 (m, 1H), 8.06 (dd, J = 8.0, 5.4 Hz, 1H), 8.02-7.95 (m, 2H). | DMSO | >98 | Method D, F3, G1 | | | |
| 49 | 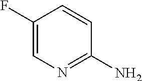 | 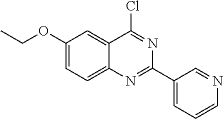 [SMILES: CCOc1ccc2nc(-c3cccnc3)nc(Cl)c2c1] | 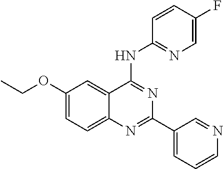 [SMILES: CCOc1ccc2nc(-c3cccnc3)nc(Nc3ccc(F)cn3)c2c1] | 2 HCl | 1H NMR (400 MHz, DMSO) δ 10.91 (s, 1H), 9.55 (d, J = 1.8 Hz, 1H), 9.15- 9.07 (m, 1H), 8.96-8.87 (m, 1H), 8.52 (d, J = 3.0 Hz, 1H), 8.44 (dd, J = 9.1, 4.1 Hz, 1H), 8.21 (d, J = 2.6 Hz, 1H), 8.04- | DMSO | >98 | Method C1, E, F3, H1 | | | |
| | | | | | 7.89 (m, 3H), 7.60 | | | | | | |
| | | | | | (dd, J = 9.1, 2.6 Hz, | | | | | | |
| | | | | | 1H), 4.27 (q, J = | | | | | | |
| | | | | | 6.9 Hz, 2H), 1.45 | | | | | | |
| | | | | | (t, J = 7.0 Hz, 3H). | | | | | | |
| 50 | 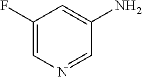 | 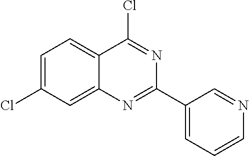 [SMILES: Clc1ccc2c(Cl)nc(-c3cccnc3)nc2c1] | 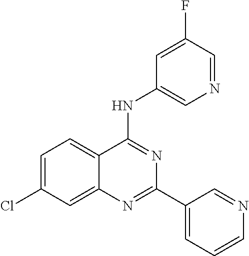 [SMILES: Fc1cncc(Nc2nc(-c3cccnc3)nc3cc(Cl)ccc23)c1] | 2 HCl | 1H NMR (400 MHz, DMSO) δ 10.79 (s, 1H), 9.52 (d, J = 1.7 Hz, 1H), 9.09 (d, J = 8.2 Hz, 1H), 9.05-9.02 (m, 1H), 8.97 (d, J = 4.3 Hz, 1H), 8.79 (d, J = 8.9 Hz, 1H), 8.46 (d, J = 2.6 Hz, 1H), 8.44-8.38 (m, 1H), 8.09- 8.02 (m, 2H), 7.83 (dd, J = 8.9, 2.2 Hz, 1H). | DMSO | >98 | Method D, F3, G1 | | | |
| 51 | 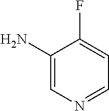 | 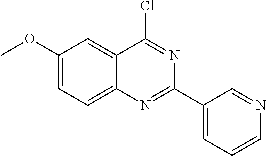 [SMILES: COc1ccc2nc(-c3cccnc3)nc(Cl)c2c1] | 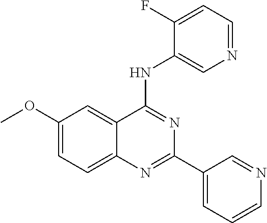 [SMILES: COc1ccc2nc(-c3cccnc3)nc(Nc3cnccc3F)c2c1] | HCl | 1H NMR (400 MHz, DMSO) δ 10.82 (s, 1H), 9.58 (s, 1H), 9.09 (d, J = 8.0 Hz, 1H), 8.91 (d, J = 5.0 Hz, 1H), 8.45 (dd, J = 8.0, 2.3 Hz, 1H), 8.20 (d, J = 2.7 Hz, 1H), 8.14 (dd, J = 16.7, 8.1 Hz, 1H), 8.00-7.90 (m, 2H), 7.61 (dd, J = 9.1, 2.7 Hz, 1H), 6.97 (dd, J = 7.9, | DMSO | >98 | Method C2, E(NaOH_{(aq.)} instead of NaOH and Δ), F2, H1 | | | |
| | | | | | 2.5 Hz, 1H), 3.99 | | | | | | |
| | | | | | (s, 3H). | | | | | | |
| 52 | 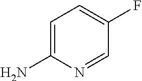 | 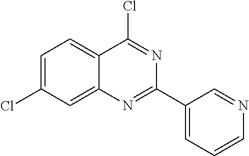 [SMILES: Clc1ccc2c(Cl)nc(-c3cccnc3)nc2c1] | 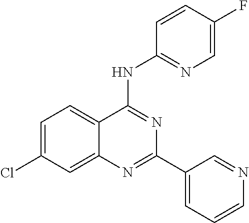 [SMILES: Fc1ccc(Nc2nc(-c3cccnc3)nc3cc(Cl)ccc23)nc1] | HCl | 1H NMR (400 MHz, DMSO) δ 10.92 (s, 1H), 9.55 (s, 1H), 9.02-8.95 (m, 1H), 8.89 (d, J = 5.0 Hz, 1H), 8.79 (d, J = 8.9 Hz, 1H), 8.51 (d, J = 3.1 Hz, 1H), 8.40 (dd, J = 9.2, 4.1 Hz, 1H), 8.01 (d, J = 2.1 Hz, 1H), 7.94 (ddd, J = 9.1, 8.3, 3.1 Hz, 1H), 7.89 (dd, J = 7.9, 5.3 Hz, | DMSO | >98 | Method D, F3, H1 | | | |
| | | | | | 1H), 7.73 (dd, J = | | | | | | |
| | | | | | 8.9, 2.2 Hz, 1H). | | | | | | |
| 53 | 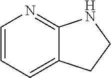 | 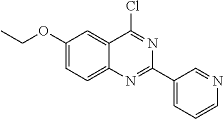 [SMILES: CCOc1ccc2nc(-c3cccnc3)nc(Cl)c2c1] | 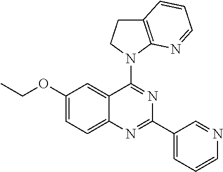 [SMILES: CCOc1ccc2nc(-c3cccnc3)nc(N3CCc4cccnc43)c2c1] | 2 HCl | 1H NMR (400 MHz, DMSO) δ 9.68 (d, J = 1.8 Hz, 1H), 9.30-9.25 (m, 1H), 8.95 (dd, J = 5.4, 1.4 Hz, 1H), 8.06 (dd, J = 8.1, 5.5 Hz, 1H), 8.01 (d, J = 9.2 Hz, 1H), 7.98- 7.94 (m, 1H), 7.79-7.73 (m, 1H), 7.66 (dd, J = | DMSO | >98 | Method C1, E, F3, H1 | | | |
| | | | | | 9.2, 2.8 Hz, 1H), | | | | | | |
| | | | | | 7.45 (d, J = 2.8 Hz, | | | | | | |
| | | | | | 1H), 6.99 (dd, J = | | | | | | |
| | | | | | 7.2, 5.2 Hz, 1H), | | | | | | |
| | | | | | 4.54 (t, J = 8.0 Hz, | | | | | | |
| | | | | | 2H), 4.03 (q, J = | | | | | | |
| | | | | | 7.0 Hz, 2H), 3.30 (t, | | | | | | |
| | | | | | J = 7.9 Hz, 2H), | | | | | | |
| | | | | | 1.35 (t, J = | | | | | | |
| | | | | | 7.0 Hz, 3H). | | | | | | |
| 54 | 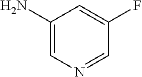 | 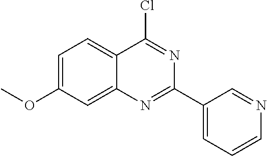 [SMILES: COc1ccc2c(Cl)nc(-c3cccnc3)nc2c1] | 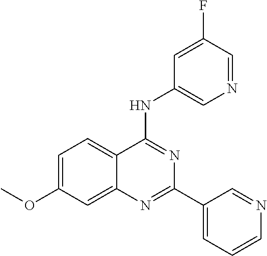 [SMILES: COc1ccc2c(Nc3cncc(F)c3)nc(-c3cccnc3)nc2c1] | 2 HCl | 1H NMR (400 MHz, DMSO) δ 10.76 (s, 1H), 9.52 (d, J = 1.8 Hz, 1H), 9.13- 9.08 (m, 1H), 9.03 (t, J = 1.6 Hz, 1H), 8.97 (dd, J = 5.4, 1.4 Hz, 1H), 8.71 (d, J = 9.2 Hz, 1H), 8.45 (d, J = 2.6 Hz, 1H), 8.43-8.38 (m, 1H), 8.05 (dd, J = 8.0, 5.4 Hz, 1H), 7.46 (d, J = 2.4 Hz, 1H), 7.40 (dd, J = 9.1, 2.6 Hz, 1H), | DMSO | >98 | Method C1, E, F3, H1 | | | |
| | | | | | 3.99 (s, 3H). | | | | | | |
| 55 | 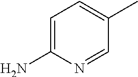 | 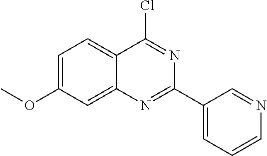 [SMILES: COc1ccc2c(Cl)nc(-c3cccnc3)nc2c1] | 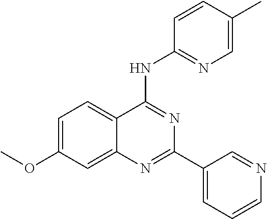 | 2 HCl | 1H NMR (400 MHz, DMSO) δ 11.43 (s, 1H), 9.58 (d, J = 1.7 Hz, 1H), 9.10 (d, J = 8.1 Hz, 1H), 8.96 (dd, J = 5.3, 1.5 Hz, 1H), 8.80 (d, J = 9.2 Hz, 1H), 8.43 (d, J = 2.2 Hz, 1H), 8.28 (d, J = 8.6 Hz, 1H), 8.05 (dd, J = 8.7, 2.0 Hz, 1H), 7.99 (dd, J = 8.0, 5.4 Hz, 1H), 7.50 (d, J = | DMSO | >98 | Method C1, E, F3, H1 | | | |
| | | | | | 2.4 Hz, 1H), 7.39 | | | | | | |
| | | | | | (dd, J = 9.2, 2.6 | | | | | | |
| | | | | | Hz, 1H), 4.00 (s, | | | | | | |
| | | | | | 3H), 2.40 (s, 3H). | | | | | | |
| 56 | 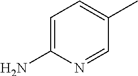 | 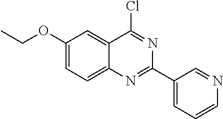 [SMILES: CCOc1ccc2nc(-c3cccnc3)nc(Cl)c2c1] | 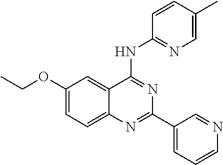 [SMILES: CCOc1ccc2nc(-c3cccnc3)nc(Nc3ccc(C)cn3)c2c1] | 2 HCl | 1H NMR (400 MHz, DMSO) δ 11.21 (s, 1H), 9.56 (d, J = 1.7 Hz, 1H), 9.09 (d, J = 8.3 Hz, 1H), 8.95-8.89 (m, 1H), 8.43-8.39 (m, 1H), 8.36 (d, J = 8.5 Hz, 1H), 8.28 (d, J = 2.4 Hz, 1H), 8.02-7.94 (m, | DMSO | >98 | Method C1, E, F3, H1 | | | |
| | | | | | 3H), 7.63 (dd, J = | | | | | | |
| | | | | | 9.1, 2.6 Hz, 1H), | | | | | | |
| | | | | | 4.30 (q, J = 6.9 | | | | | | |
| | | | | | Hz, 2H), 2.39 (s, | | | | | | |
| | | | | | 3H), 1.45 (t, J = 7.0 | | | | | | |
| | | | | | Hz, 3H). | | | | | | |
| 57 | 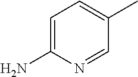 | 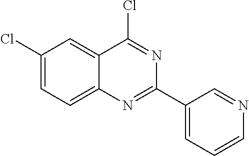 [SMILES: Clc1ccc2nc(-c3cccnc3)nc(Cl)c2c1] | 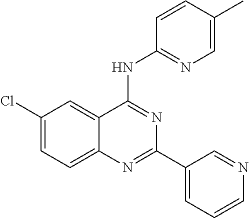 [SMILES: Cc1ccc(Nc2nc(-c3cccnc3)nc3ccc(Cl)cc23)nc1] | | 1H NMR (400 MHz, DMSO) δ 10.61 (s, 1H), 9.54 (dd, J = 2.1, 0.7 Hz, 1H), 8.97-8.94 (m, 1H), 8.74-8.66 (m, 2H), 8.35-8.30 (m, 2H), 7.94- 7.91 (m, 2H), 7.82 (dd, J = 8.6, 2.2 Hz, 1H), 7.60-7.54 (m, 1H), 2.35 (s, 3H). | DMSO | >98 | Method D, F3, H1 | | | |
| 58 | 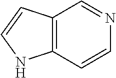 | 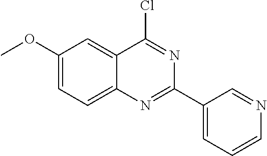 [SMILES: COc1ccc2nc(-c3cccnc3)nc(Cl)c2c1] | 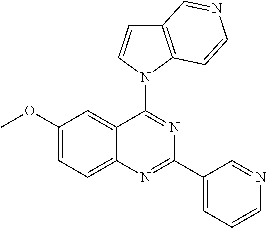 [SMILES: COc1ccc2nc(-c3cccnc3)nc(-n3ccc4cnccc43)c2c1] | 2 HCl | 1H NMR (400 MHz, DMSO) δ 9.68 (d, J = 1.6 Hz, 1H), 9.16-9.11 (m, 1H), 9.00-8.92 (m, 3H), 8.89 (dd, J = 5.6, 1.0 Hz, 1H), 8.28 (d, J = 9.2 Hz, 1H), 7.96 (dd, J = 8.1, 5.2 Hz, 1H), 7.90 (dd, J = 9.2, 2.7 Hz, 1H), 7.85 (dd, J = 8.4, 5.6 Hz, 1H), 7.40 (d, J = 2.7 Hz, 1H), 7.35 | DMSO | >98 | Method C2, E(NaOH_{(aq.)} instead of NaOH and Δ), F2, G2 | | | |
| | | | | | (dd, J = 3.6, 0.7 | | | | | | |
| | | | | | Hz, 1H), | | | | | | |
| | | | | | 3.91 (s, 3H). | | | | | | |
| 59 | 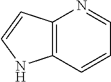 | 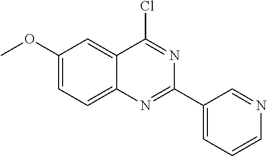 [SMILES: COc1ccc2nc(-c3cccnc3)nc(Cl)c2c1] | 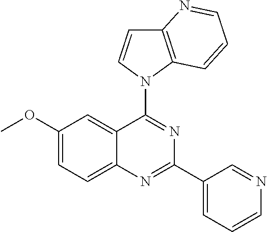 [SMILES: COc1ccc2nc(-c3cccnc3)nc(-n3ccc4ncccc43)c2c1] | 2 HCl | 1H NMR (400 MHz, DMSO) δ 9.67 (s, 1H), 9.56 (s, 1H), 9.18-9.12 (m, 1H), 8.95 (d, J = 4.5 Hz, 1H), 8.70 (d, J = 3.5 Hz, 1H), 8.68 (d, J = 7.0 Hz, 1H), 8.43 (d, J = 6.7 Hz, 1H), 8.30 (d, J = 9.3 Hz, 1H), 7.97 (dd, J = 8.0, 5.2 Hz, 1H), 7.91 (dd, J = 9.3, 2.7 Hz, 1H), 7.50 | DMSO | >98 | Method C2, E(NaOH_{(aq.)} instead of NaOH and Δ), F2, G2 | | | |
| | | | | | (dd, J = 3.5, 0.6 | | | | | | |
| | | | | | Hz, 1H), 7.31 (d, | | | | | | |
| | | | | | J = 2.7 Hz, 1H), | | | | | | |
| | | | | | 3.90 (s, 3H). | | | | | | |
| 60 | 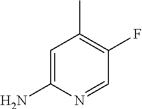 | 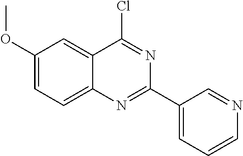 [SMILES: COc1ccc2nc(-c3cccnc3)nc(Cl)c2c1] | 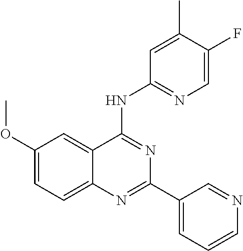 [SMILES: COc1ccc2nc(-c3cccnc3)nc(Nc3cc(C)c(F)cn3)c2c1] | HCl | 1H NMR (400 MHz, DMSO) δ 10.78 (s, 1H), 9.52 (d, J = 1.7 Hz, 1H), 9.09- 9.04 (m, 1H), 8.92 (dd, J = 5.3, 1.4 Hz, 1H), 8.41-8.36 (m, 2H), 8.19 (d, J = 2.6 Hz, 1H), 7.98 (dd, J = 8.0, 5.4 Hz, 1H), 7.92 (d, J = 9.1 Hz, 1H), 7.60 (dd, J = 9.1, 2.7 Hz, 1H), 3.98 (s, 3H), 2.45 (s, 3H). | DMSO | >98 | Method C2, E(NaOH_{(aq.)} instead of NaOH and Δ), F2, G2 | | | |
| 61 | 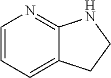 | 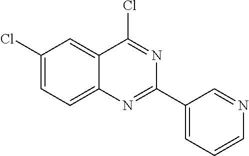 [SMILES: Clc1ccc2nc(-c3cccnc3)nc(Cl)c2c1] | 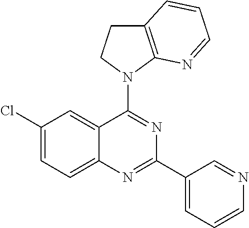 [SMILES: Clc1ccc2nc(-c3cccnc3)nc(N3CCc4cccnc43)c2c1] | 2 HCl | 1H NMR (400 MHz, DMSO) δ 9.70 (d, J = 1.7 Hz, 1H), 9.27-9.22 (m, 1H), 8.97 (dd, J = 5.3, 1.4 Hz, 1H), 8.20- 8.18 (m, 1H), 8.09-7.99 (m, 3H), 7.95 (dd, J = 5.1, 1.6 Hz, 1H), 7.77 (dd, J = 7.3, 1.5 Hz, 1H), 7.02 (dd, J = 7.3, 5.1 Hz, 1H), 4.53 (t, J = 8.0 Hz, 2H), 3.29 (t, | DMSO | >98 | Method D, F3, H1 | | | |
| | | | | | J = 7.9 Hz, 2H). | | | | | | |
| 62 | 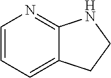 | 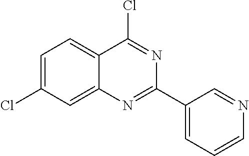 [SMILES: Clc1ccc2c(Cl)nc(-c3cccnc3)nc2c1] | 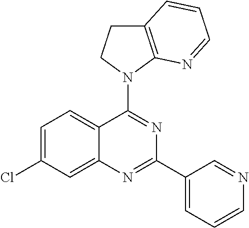 [SMILES: Clc1ccc2c(N3CCc4cccnc43)nc(-c3cccnc3)nc2c1] | 2 HCl | 1H NMR (400 MHz, DMSO) δ 9.69 (d, J = 1.6 Hz, 1H), 9.28-9.23 (m, 1H), 8.99 (d, J = 4.2 Hz, 1H), 8.18 (d, J = 9.0 Hz, 1H), 8.10 (d, J = 2.1 Hz, 1H), 8.06 (dd, J = 8.1, 5.4 Hz, 1H), 7.96- 7.92 (m, 1H), 7.77 (dd, J = 7.3, 1.4 Hz, 1H), 7.63 (dd, J = 9.0, 2.2 Hz, 1H), 7.02 (dd, J = | DMSO | >98 | Method D, F3, H1 | | | |
| | | | | | 7.3, 5.1 Hz, 1H), | | | | | | |
| | | | | | 4.53 (t, J = 8.0 | | | | | | |
| | | | | | Hz, 2H), 3.29 (t, J = | | | | | | |
| | | | | | 7.9 Hz, 2H). | | | | | | |
| 63 | 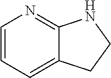 | 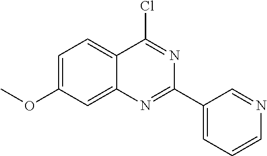 [SMILES: COc1ccc2c(Cl)nc(-c3cccnc3)nc2c1] | 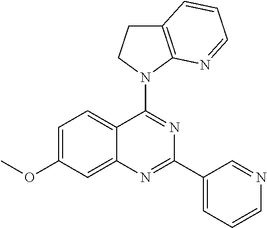 [SMILES: COc1ccc2c(N3CCc4cccnc43)nc(-c3cccnc3)nc2c1] | | 1H NMR (400 MHz, DMSO) δ 9.66- 9.63 (m, 1H), 8.81- 8.76 (m, 1H), 8.72 (dd, J = 4.8, 1.7 Hz, 1H), 7.97 (d, J = 9.2 Hz, 1H), 7.87 (dd, J = 5.1, 1.5 Hz, 1H), 7.68 (dd, J = 7.2, 1.5 Hz, 1H), 7.58 (ddd, J = 8.0, 4.8, 0.8 Hz, 1H), 7.37 (d, J = 2.6 Hz, 1H), 7.14 (dd, J = 9.2, | DMSO | >98 | Method C1, E, F3, H1 | | | |
| | | | | | 2.6 Hz, 1H), 6.91 | | | | | | |
| | | | | | (dd, J = 7.2, 5.1 | | | | | | |
| | | | | | Hz, 1H), 4.46 (t, J = | | | | | | |
| | | | | | 8.0 Hz, 2H), 4.00 | | | | | | |
| | | | | | (s, 3H), 3.24 (t, J = | | | | | | |
| | | | | | 8.0 Hz, 2H). | | | | | | |
| 64 | 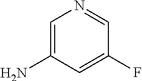 | 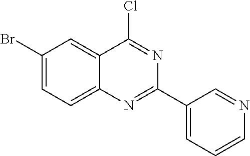 [SMILES: Clc1nc(-c2cccnc2)nc2ccc(Br)cc12] | 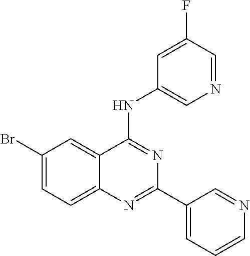 [SMILES: Fc1cncc(Nc2nc(-c3cccnc3)nc3ccc(Br)cc23)c1] | | 1H NMR (400 MHz, DMSO) δ 10.25 (s, 1H), 9.49 (d, J = 1.5 Hz, 1H), 8.86 (d, J = 2.1 Hz, 1H), 8.75-8.72 (m, 1H), 8.71 (dd, J = 4.7, 1.7 Hz, 1H), 8.66-8.61 (m, 1H), 8.48 (ddd, J = 8.8, 7.4, 2.8 Hz, 1H), 8.05 (dd, J = 8.9, 2.1 Hz, 1H), 7.86 (d, J = 8.9 Hz, 1H), 7.56 (dd, J = 7.3, 4.8 Hz, 1H), 7.35 (dd, J = 8.8, | DMSO | >98 | Method D, F3, G1 | | | |
| | | | | | 3.2 Hz, 1H). | | | | | | |
| 65 | 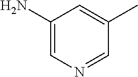 | 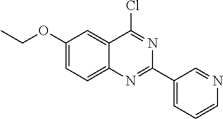 [SMILES: CCOc1ccc2nc(-c3cccnc3)nc(Cl)c2c1] | 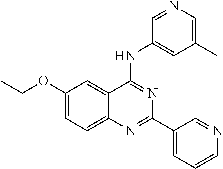 [SMILES: CCOc1ccc2nc(-c3cccnc3)nc(Nc3cncc(C)c3)c2c1] | 2 HCl | 1H NMR (400 MHz, DMSO) δ 11.06 (s, 1H), 9.58-9.48 (m, 2H), 9.12- 9.00 (m, 1H), 8.92 (s, 1H), 8.89 (dd, J = 5.2, 1.5 Hz, 1H), 8.58 (s, 1H), 8.30 (d, J = 2.6 Hz, 1H), 8.01-7.87 (m, 2H), 7.64 (dd, | DMSO | >98 | Method C1, E, F3, H1 | | | |
| | | | | | J = 9.1, 2.6 Hz, | | | | | | |
| | | | | | 1H), 4.32 (q, J = 7.0 | | | | | | |
| | | | | | Hz, 2H), 2.57 (s, | | | | | | |
| | | | | | 3H), 1.45 (t, J = | | | | | | |
| | | | | | 7.0 Hz, 3H). | | | | | | |
| 66 | 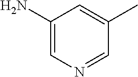 | 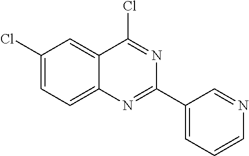 [SMILES: Clc1ccc2nc(-c3cccnc3)nc(Cl)c2c1] | 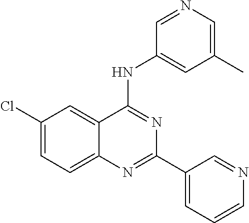 [SMILES: Cc1cncc(Nc2nc(-c3cccnc3)nc3ccc(Cl)cc23)c1] | 2 HCl | 1H NMR (400 MHz, DMSO) δ 10.99 (s, 1H), 9.55 (d, J = 1.5 Hz, 1H), 9.45 (d, J = 1.8 Hz, 1H), 9.03-8.93 (m, 2H), 8.88 (dd, J = 5.1, 1.5 Hz, 1H), 8.79 (s, 1H), 8.59 (s, 1H), 8.07- 7.97 (m, 2H), 7.86 (dd, J = 8.1, 5.1 Hz, 1H), 2.56 (s, 3H). | DMSO | >98 | Method D, F3, H1 | | | |
| 67 | 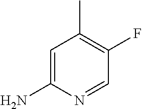 | 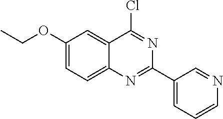 [SMILES: CCOc1ccc2nc(-c3cccnc3)nc(Cl)c2c1] | 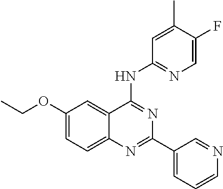 [SMILES: CCOc1ccc2nc(-c3cccnc3)nc(Nc3cc(C)c(F)cn3)c2c1] | 2 HCl | 1H NMR (400 MHz, DMSO) δ 10.84 (s, 1H), 9.52 (d, J = 1.8 Hz, 1H), 9.16 (d, J = 8.3 Hz, 1H), 8.97 (dd, J = 5.4, 1.3 Hz, 1H), 8.40 (d, J = 0.9 Hz, 1H), 8.36 (d, J = 5.9 Hz, 1H), 8.21 (d, J = 2.6 Hz, 1H), 8.07 (dd, J = 8.1, 5.4 Hz, 1H), 7.95 (d, | DMSO | >98 | Method C1, E, F3, H1 | | | |
| | | | | | J = 9.1 Hz, 1H), | | | | | | |
| | | | | | 7.60 (dd, J = 9.1, | | | | | | |
| | | | | | 2.6 Hz, 1H), 4.27 | | | | | | |
| | | | | | (q, J = 7.0 Hz, 2H), | | | | | | |
| | | | | | 2.46 (d, J = 0.9 | | | | | | |
| | | | | | Hz, 3H), 1.45 (t, | | | | | | |
| | | | | | J = 7.0 Hz, 3H). | | | | | | |
| 68 | 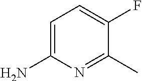 | 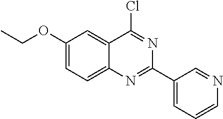 [SMILES: CCOc1ccc2nc(-c3cccnc3)nc(Cl)c2c1] | 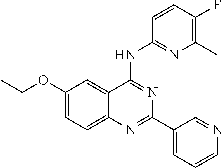 [SMILES: CCOc1ccc2nc(-c3cccnc3)nc(Nc3ccc(F)c(C)n3)c2c1] | 2 HCl | 1H NMR (400 MHz, DMSO) δ 10.91 (s, 1H), 9.57-9.49 (m, 1H), 9.16 (d, J = 8.1 Hz, 1H), 8.97 (d, J = 5.3 Hz, 1H), 8.29-8.16 (m, 2H), 8.10-8.01 (m, 1H), 7.97 (d, J = 9.1 Hz, 1H), 7.90-7.76 (m, | DMSO | >98 | Method C1, E, F3, H1 | | | |
| | | | | | 1H), 7.59 (dd, J = | | | | | | |
| | | | | | 9.1, 2.4 Hz, 1H), | | | | | | |
| | | | | | 4.27 (q, J = 6.9 Hz, | | | | | | |
| | | | | | 2H), 2.53-2.49 (m, | | | | | | |
| | | | | | 3H), 1.45 (t, J = | | | | | | |
| | | | | | 7.0 Hz, 3H). | | | | | | |
| 69 | 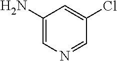 | 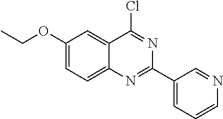 [SMILES: CCOc1ccc2nc(-c3cccnc3)nc(Cl)c2c1] | 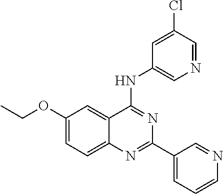 [SMILES: CCOc1ccc2nc(-c3cccnc3)nc(Nc3cncc(Cl)c3)c2c1] | 2 HCl | 1H NMR (400 MHz, DMSO) δ 10.64 (s, 1H), 9.53-9.44 (m, 1H), 9.17 (d, J = 2.0 Hz, 1H), 9.08 (d, J = 8.1 Hz, 1H), 8.94 (d, J = 5.3 Hz, 1H), 8.62- 8.53 (m, 1H), 8.48 (d, J = 2.2 Hz, 1H), 8.23-8.15 (m, 1H), 8.09-8.00 (m, 1H), 7.96 (d, | DMSO | >98 | Method C1, E, F3, H1 | | | |
| | | | | | J = 9.1 Hz, 1H), | | | | | | |
| | | | | | 7.64 (dd, J = 9.1, | | | | | | |
| | | | | | 2.6 Hz, 1H), 4.30 | | | | | | |
| | | | | | (q, J = 7.0 Hz, 2H), | | | | | | |
| | | | | | 1.46 (t, J = 7.0 | | | | | | |
| | | | | | Hz, 3H). | | | | | | |
| 70 | 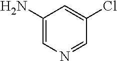 | 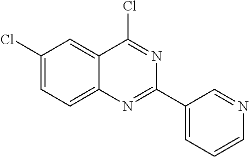 [SMILES: Clc1ccc2nc(-c3cccnc3)nc(Cl)c2c1] | 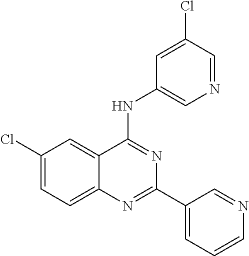 [SMILES: Clc1cncc(Nc2nc(-c3cccnc3)nc3ccc(Cl)cc23)c1] | 2 HCl | 1H NMR (400 MHz, DMSO) δ 10.52 (s, 1H), 9.50 (d, J = 1.5 Hz, 1H), 9.12 (d, J = 2.1 Hz, 1H), 8.98-8.92 (m, 1H), 8.89 (dd, J = 5.2, 1.5 Hz, 1H), 8.84-8.80 (m, 1H), 8.62-8.50 (m,, 1H), 8.47 (d, J = 2.2 Hz, 1H), 8.01- 7.94 (m, 2H), 7.91 (dd, J = 8.0, 5.2 Hz, 1H). | DMSO | >98 | Method D, F3, H1 | | | |
| 71 | 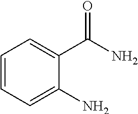 | 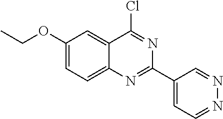 [SMILES: CCOc1ccc2nc(-c3ccnnc3)nc(Cl)c2c1] | 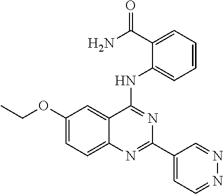 [SMILES: CCOc1ccc2nc(-c3ccnnc3)nc(Nc3ccccc3C(N)=O)c2c1] | HCl | ¹H NMR (400 MHz, DMSO) δ 13.06 (s, 1H), 10.07 (dd, J = 2.2, 1.3 Hz, 1H), 9.47 (dd, J = 5.3, 1.2 Hz, 1H), 9.05 (d, J = 8.4 Hz, 1H), 8.61-8.33 (m, 2H), 7.97 (dd, J = 11.9, 5.2 Hz, 3H), 7.80-7.71 (m, 1H), 7.67-7.56 (m, 2H), 7.28-7.19 (m, | DMSO | >98 | Method I, J, K, E G1 | | | |
| | | | | | 1H), 4.26 (q, J = | | | | | | |
| | | | | | 6.9 Hz, 2H), 1.47 | | | | | | |
| | | | | | (t, J = 6.9 Hz, | | | | | | |
| | | | | | 3H). | | | | | | |

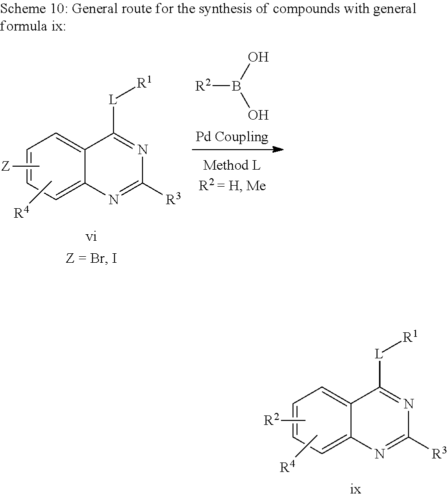

### Method L: Pd(PPh₃)₄/K₃PO₄/Dioxane—H₂O, heat

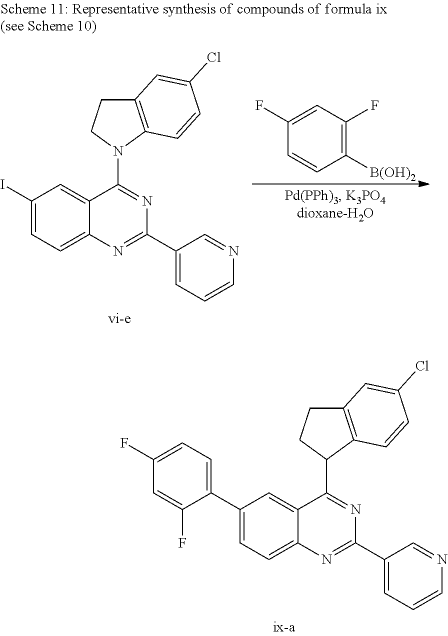 [SMILES: Clc1ccc2c(c1)CCN2c1nc(-c2cccnc2)nc2ccc(I)cc12 ; OB(O)c1ccc(F)cc1F ; Fc1ccc(-c2ccc3nc(-c4cccnc4)nc(C4CCc5cc(Cl)ccc54)c3c2)c(F)c1]

### Method L: 4-(5-chloroindolin-1-yl)-6-(2,4-difluorophenyl)-2-(pyridin-3-yl)quinazoline (ix-a)

To a mixture of 4-(5-chloroindolin-1-yl)-6-iodo-2-(pyridin-3-yl)quinazoline (0.25 g, 0.516 mmol), 2,4-difluorophenylboronic acid (0.122 g, 0.774 mmol) and K₃PO₄ (0.328 g, 1.547 mmol) in dioxane (15 ml)—H₂O (3 ml) was added Pd(Ph₃P)₄ (0.060 g, 0.052 mmol). The reaction mixture was stirred under N₂ at −90-100° C. for 5 h and cooled to room temperature. The reaction was diluted with 10 mL of ethyl acetate and 10 mL of water to give crude 4-(5-chloroindolin-1-yl)-6-(2,4-difluorophenyl)-2-(pyridin-3-yl)quinazoline upon sonication. The resulting precipitate was filtered off and subsequently dissolved in 30 mL of DMF. To this DMF solution was added NH—SiO₂ (1.0 g) and sonicated. The silica was filtered off to remove Pd black and the filtrate was evaporated in vacuo to give a pale yellow solid which was washed with ethanol and dried to give 4-(5-chloroindolin-1-yl)-6-(2,4-difluorophenyl)-2-(pyridin-3-yl)quinazoline (0.20 g, 0.42 mmol, 82.35% yield) as a pale yellow powder. ¹H NMR (400 MHz, DMSO) δ 9.57 (s, 1H), 8.71 (d, J=5.4 Hz, 2H), 8.30 (s, 1H), 8.17-8.00 (m, 2H), 7.92-7.66 (m, 2H), 7.64-7.53 (m, 1H), 7.46 (d, J=16.4 Hz, 2H), 7.40-7.17 (m, 2H), 4.66 (t, J=7.5 Hz, 2H), 3.30-3.10 (m, 2H).

The compounds in the following table were prepared in a manner analogous to that described in Scheme 11 substituting with appropriate boronic acid

TABLE 2

| Num- | Starting | Starting | | Salt | |
|---|---|---|---|---|---|
| ber | Material 1 | Material 2 | Product | type | ¹H NMR |
| 72 | 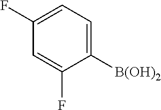 [SMILES: OB(O)c1ccc(F)cc1F] | 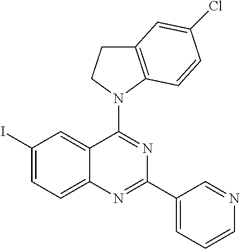 [SMILES: Clc1ccc2c(c1)CCN2c1nc(-c2cccnc2)nc2ccc(I)cc12] | 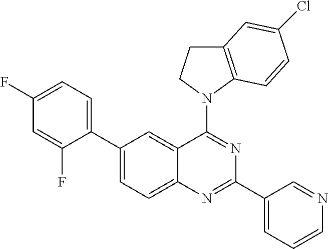 [SMILES: Fc1ccc(-c2ccc3nc(-c4cccnc4)nc(N4CCc5cc(Cl)ccc54)c3c2)c(F)c1] | | 1H NMR (400 MHz, DMSO) δ 9.57 (s, 1H), 8.71 (d, J = 5.4 Hz, 2H), 8.30 (s, 1H), 8.17 8.00 (m, 2H), 7.92-7.66 (m, 2H), 7.64- 7.53 (m, 1H), 7.46 (d, J = 16.4 Hz, 2H), 7.40- 7.17 (m, 2H), 4.66 (t, J = 7.5 Hz, 2H), 3.30- 3.10 (m, 2H). |
| 73 | 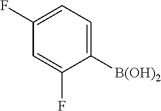 [SMILES: OB(O)c1ccc(F)cc1F] | 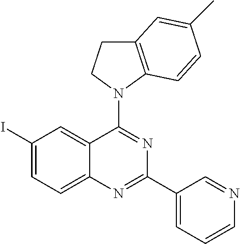 [SMILES: Cc1ccc2c(c1)CCN2c1nc(-c2cccnc2)nc2ccc(I)cc12] | 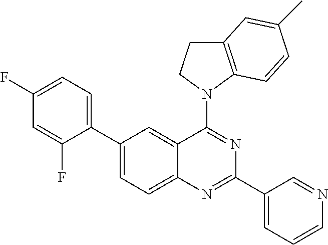 [SMILES: Cc1ccc2c(c1)CCN2c1nc(-c2cccnc2)nc2ccc(-c3ccc(F)cc3F)cc12] | | 1H NMR (400 MHz, DMSO) δ 9.58 (dd, J = 2.1, 0.8 Hz, 1H), 8.77-8.66 (m, 2H), 8.30 (s, 1H), 8.11-7.96 (m, 2H), 7.82-7.69 (m, 2H), 7.57 (ddd, J = 7.9, 4.9, 0.8 Hz, 1H), 7.44 (ddd, J = 11.6, 9.3, 2.6 Hz, 1H), 7.31- 7.21 (m, 1H), 7.18 (s, 1H), 7.09 (d, J = 8.2 Hz, 1H), 4.62 (t, J = 7.9 Hz, 2H), 3.18 (t, J = 7.8 Hz, 2H), 2.33 (s, 3H). |
| 74 | 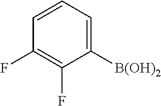 [SMILES: OB(O)c1cccc(F)c1F] | 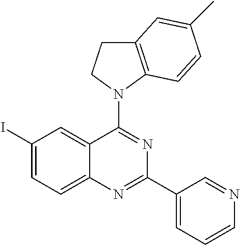 [SMILES: Cc1ccc2c(c1)CCN2c1nc(-c2cccnc2)nc2ccc(I)cc12] | 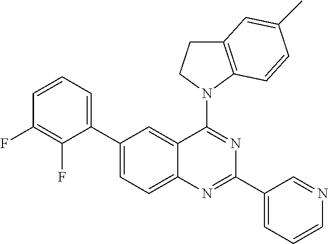 [SMILES: Cc1ccc2c(c1)CCN2c1nc(-c2cccnc2)nc2ccc(-c3cccc(F)c3F)cc12] | | 1H NMR (400 MHz, DMSO) δ 9.58 (dd, J = 2.1, 0.8 Hz, 1H), 8.79-8.67 (m, 2H), 8.36 (s, 1H), 8.16-8.05 (m, 1H), 8.02 (d, J = 8.7 Hz, 1H), 7.76 (d, J = 8.2 Hz, 1H), 7.62- 7.44 (m, 3H), 7.41-7.31 (m, 1H), 7.18 (s, 1H), 7.10 (d, J = 8.3 Hz, 1H), 4.63 (t, J = 7.9 Hz, 2H), 3.19 (t, J = 7.8 Hz, 2H), 2.33 (s, 3H). |
| 75 | 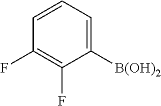 [SMILES: OB(O)c1cccc(F)c1F] | 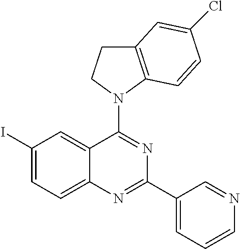 [SMILES: Clc1ccc2c(c1)CCN2c1nc(-c2cccnc2)nc2ccc(I)cc12] | 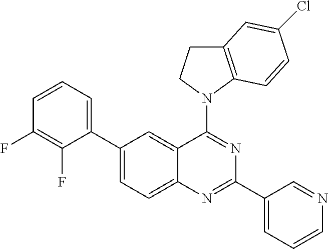 [SMILES: Fc1cccc(-c2ccc3nc(-c4cccnc4)nc(N4CCc5cc(Cl)ccc54)c3c2)c1F] | | 1H NMR (400 MHz, DMSO) δ 9.55 (d, J = 1.4 Hz, 1H), 8.77-8.65 (m, 2H), 8.32 (s, 1H), 8.09 (d, J = 8.7 Hz, 1H), 8.02 (d, J = 8.7 Hz, 1H), 7.83 (d, J = 8.6 Hz, 1H), 7.66-7.46 (m, 3H), 7.42 (d, J = 2.1 Hz, 1H), 7.41- 7.28 (m, 2H), 4.65 (t, J = 8.0 Hz, 2H), 3.23 (t, J = 7.9 Hz, 2H). |
| 76 | 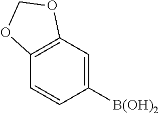 [SMILES: OB(O)c1ccc2c(c1)OCO2] | 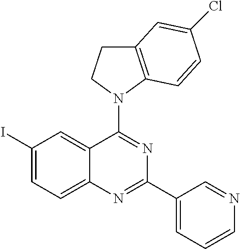 [SMILES: Clc1ccc2c(c1)CCN2c1nc(-c2cccnc2)nc2ccc(I)cc12] | 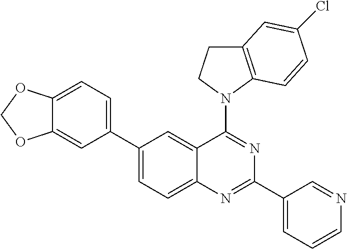 [SMILES: Clc1ccc2c(c1)CCN2c1nc(-c2cccnc2)nc2ccc(-c3ccc4c(c3)OCO4)cc12] | | 1H NMR (400 MHz, DMSO) δ 9.56 (d, J = 0.9 Hz, 1H), 8.75-8.64 (m, 2H), 8.27 (d, J = 1.8 Hz, 1H), 8.18 (dd, J = 8.8, 2.0 Hz, 1H), 7.98 (d, J = 8.7 Hz, 1H), 7.76 (d, J = 8.6 Hz, 1H), 7.63-7.51 (m, 1H), 7.42 (dd, J = 17.9, 1.9 Hz, 2H), 7.37-7.23 (m, 2H), 7.05 (d, J = 8.1 Hz, 1H), 6.10 (s, 2H), 4.69 (t, J = 8.0 Hz, 2H), 3.33 (s, 3H), 3.25 (t, J = 7.9 Hz, 2H). |
| 77 | 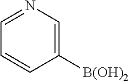 | 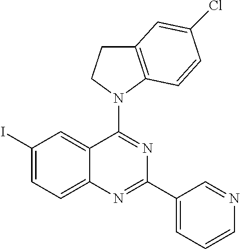 [SMILES: Clc1ccc2c(c1)CCN2c1nc(-c2cccnc2)nc2ccc(I)cc12] | 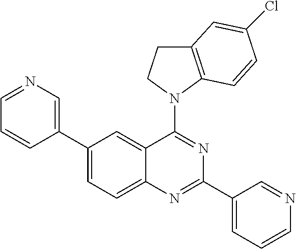 [SMILES: Clc1ccc2c(c1)CCN2c1nc(-c2cccnc2)nc2ccc(-c3cccnc3)cc12] | | 1H NMR (400 MHz, DMSO) δ 9.58 (d, J = 2.1 Hz, 1H), 9.05 (d, J = 1.8 Hz, 1H), 8.76- 8.69 (m, 2H), 8.64 (dd, J = 4.7, 1.5 Hz, 1H), 8.46 (d, J = 1.9 Hz, 1H), 8.30 (dd, J = 8.7, 2.0 Hz, 1H), 8.28-8.21 (m, 1H), 8.07 (d, J = 8.7 Hz, 1H), 7.85 (d, J = 8.6 Hz, 1H), 7.62- 7.52 (m, 2H), 7.46 (d, J = 2.2 Hz, 1H), 7.36 (dd, J = 8.6, 2.3 Hz, 1H), 4.75 (t, J = 8.0 Hz, 2H), 3.26 (t, J = 7.8 Hz, 2H). |
| 78 | 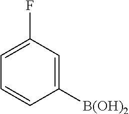 | 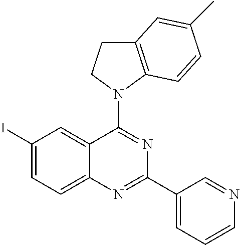 [SMILES: Cc1ccc2c(c1)CCN2c1nc(-c2cccnc2)nc2ccc(I)cc12] | 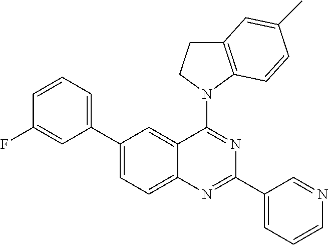 [SMILES: Cc1ccc2c(c1)CCN2c1nc(-c2cccnc2)nc2ccc(-c3cccc(F)c3)cc12] | | 1H NMR (400 MHz, DMSO) δ 9.58 (dd, J = 2.1, 0.7 Hz, 1H), 8.77-8.67 (m, 2H), 8.42 (d, J = 1.9 Hz, 1H), 8.30-8.20 (m, 1H), 8.01 (d, J = 8.7 Hz, 1H), 7.74 (d, J = 8.2 Hz, 1H), 7.71-7.63 (m, 2H), 7.63-7.50 (m, 2H), 7.32-7.23 (m, 1H), 7.20 (s, 1H), 7.11 (d, J = 8.3 Hz, 1H), 4.69 (t, J = 7.9 Hz, 2H), 3.20 (t, J = 7.8 Hz, 2H), 2.34 (s, 3H). |
| 79 | 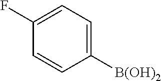 | 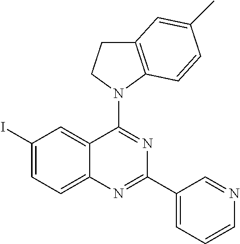 [SMILES: Cc1ccc2c(c1)CCN2c1nc(-c2cccnc2)nc2ccc(I)cc12] | 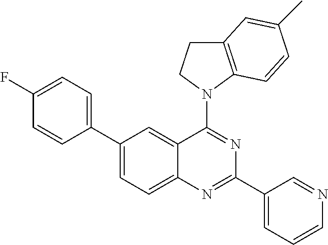 [SMILES: Cc1ccc2c(c1)CCN2c1nc(-c2cccnc2)nc2ccc(-c3ccc(F)cc3)cc12] | 2HCl | 1H NMR (400 MHz, DMSO) δ 9.58 (d, J = 1.6 Hz, 1H), 9.11 (d, J = 8.1 Hz, 1H), 8.95 (dd, J = 5.2, 1.3 Hz, 1H), 8.49-8.41 (m, 1H), 8.29 (dd, J = 8.8, 1.8 Hz, 1H), 8.16 (d, J = 8.7 Hz, 1H), 7.98 (dd, J = 7.9, 5.3 Hz, 1H), 7.94- 7.82 (m, 3H), 7.44-7.33 (m, 2H), 7.23 (d, J = 8.1 Hz, 1H), 7.14 (d, J = 8.2 Hz, 1H), 4.77 (dd, J = 15.6, 7.8 Hz, 2H), 3.22 (t, J = 7.5 Hz, 2H), 2.35 (s, 3H). |
| 80 | 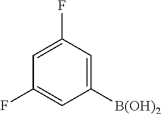 [SMILES: OB(O)c1cc(F)cc(F)c1] | 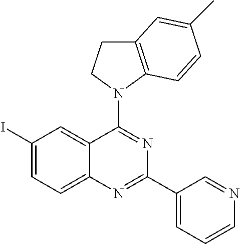 [SMILES: Cc1ccc2c(c1)CCN2c1nc(-c2cccnc2)nc2ccc(I)cc12] | 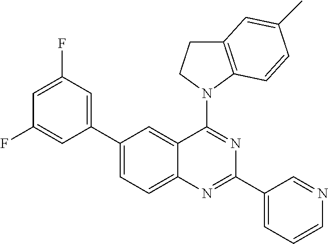 [SMILES: Cc1ccc2c(c1)CCN2c1nc(-c2cccnc2)nc2ccc(-c3cc(F)cc(F)c3)cc12] | | 1H NMR (400 MHz, DMSO) δ 9.59-9.55 (m, 1H), 8.75-8.68 (m, 2H), 8.45 (d, J = 1.9 Hz, 1H), 8.26 (dd, J = 8.8, 2.0 Hz, 1H), 8.00 (d, J = 8.8 Hz, 1H), 7.78 (d, J = 8.2 Hz, 1H), 7.64-7.54 (m, 3H), 7.34-7.25 (m, 1H), 7.20 (s, 1H), 7.12 (d, J = 8.1 Hz, 1H), 4.71 (t, J = 7.9 Hz, 2H), 3.20 (t, J = 7.9 Hz, 2H), 2.34 (s, 3H). |
| 81 | 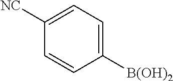 [SMILES: N#Cc1ccc(B(O)O)cc1] | 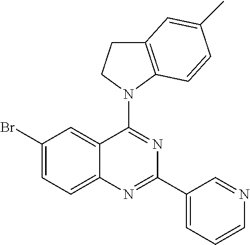 [SMILES: Cc1ccc2c(c1)CCN2c1nc(-c2cccnc2)nc2ccc(Br)cc12] | 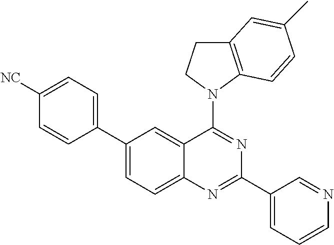 [SMILES: Cc1ccc2c(c1)CCN2c1nc(-c2cccnc2)nc2ccc(-c3ccc(C#N)cc3)cc12] | | 1H NMR (400 MHz, DMSO) δ 9.65-9.51 (m, 1H), 8.79-8.69 (m, 2H), 8.48 (s, 1H), 8.27 (d, J = 8.6 Hz, 1H), 8.12-7.91 (m, 5H), 7.77 (d, J = 7.7 Hz, 1H), 7.66-7.52 (m, 1H), 7.20 (s, 1H), 7.11 (d, J = 7.6 Hz, 1H), 4.81-4.64 (m, 2H), 3.26-3.12 (m, 2H), 2.34 (s, 3H). |
| 82 | 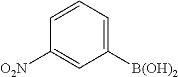 [SMILES: O=[N+]([O-])c1cccc(B(O)O)c1] | 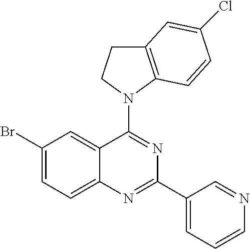 [SMILES: Clc1ccc2c(c1)CCN2c1nc(-c2cccnc2)nc2ccc(Br)cc12] | 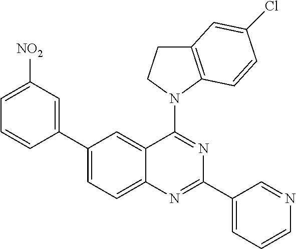 [SMILES: O=[N+]([O-])c1cccc(-c2ccc3nc(-c4cccnc4)nc(N4CCc5cc(Cl)ccc54)c3c2)c1] | | 1H NMR (400 MHz, DMSO) δ 9.58-9.48 (m, 1H), 8.76-8.62 (m, 2H), 8.56-8.47 (m, 1H), 8.44-8.34 (m, 1H), 8.32-8.19 (m, 3H), 8.05-7.94 (m, 1H), 7.85-7.74 (m, 2H), 7.59-7.48 (m, 1H), 7.42 (d, J = 2.1 Hz, 1H), 7.32 (dd, J = 8.6, 2.3 Hz, 1H), 4.67 (t, J = 8.0 Hz, 2H), 3.30-3.14 (m, 2H). |
| 83 | 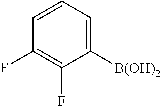 [SMILES: OB(O)c1cccc(F)c1F] | 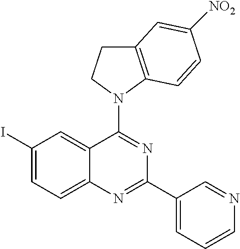 [SMILES: O=[N+]([O-])c1ccc2c(c1)CCN2c1nc(-c2cccnc2)nc2ccc(I)cc12] | 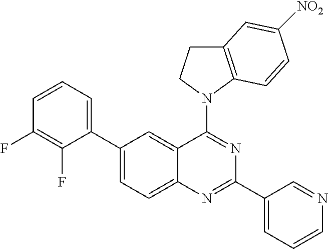 [SMILES: O=[N+]([O-])c1ccc2c(c1)CCN2c1nc(-c2cccnc2)nc2ccc(-c3cccc(F)c3F)cc12] | | 1H NMR (400 MHz, DMSO) δ 9.61 (s, 1H), 8.81-8.68 (m, 2H), 8.38 (s, 1H), 8.30- 8.11 (m, 4H), 7.83 (d, J = 8.9 Hz, 1H), 7.69- 7.47 (m, 3H), 7.45-7.30 (m, 1H), 4.76 (t, J = 8.1 Hz, 2H), 3.34 (d, J = 14.6 Hz, 14H). |
| 84 | 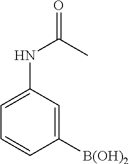 | 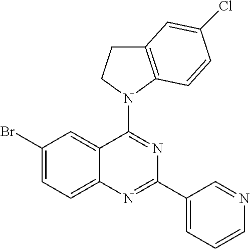 [SMILES: Clc1ccc2c(c1)CCN2c1nc(-c2cccnc2)nc2ccc(Br)cc12] | 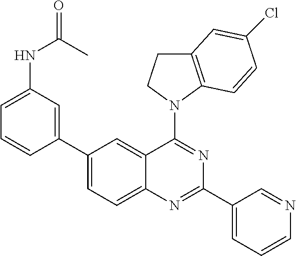 [SMILES: CC(=O)Nc1cccc(-c2ccc3nc(-c4cccnc4)nc(N4CCc5cc(Cl)ccc54)c3c2)c1] | | 1H NMR (400 MHz, DMSO) δ 10.07 (s, 1H), 9.65-9.49 (m, 1H), 8.79-8.64 (m, 2H), 8.33 (d, J = 1.8 Hz, 1H), 8.16 (dd, J = 8.7, 1.9 Hz, 1H), 8.10-7.98 (m, 2H), 7.77 (d, J = 8.6 Hz, 1H), 7.67-7.54 (m, 2H), 7.52- 7.40 (m, 3H), 7.40-7.30 (m, 1H), 4.70 (t, J = 8.1 Hz, 2H), 3.29-3.20 (m, 2H), 2.09 (s, 3H). |
| 85 | 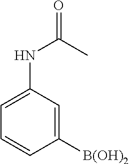 | 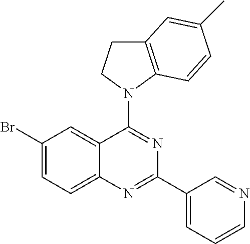 [SMILES: Cc1ccc2c(c1)CCN2c1nc(-c2cccnc2)nc2ccc(Br)cc12] | 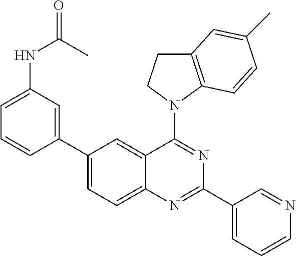 [SMILES: CC(=O)Nc1cccc(-c2ccc3nc(-c4cccnc4)nc(N4CCc5cc(C)ccc54)c3c2)c1] | | 1H NMR (400 MHz, DMSO) δ 10.06 (d, J = 9.8 Hz, 1H), 9.64-9.52 (m, 1H), 8.80-8.67 (m, 2H), 8.33 (d, J = 1.6 Hz, 1H), 8.13 (dd, J = 8.7, 1.8 Hz, 1H), 8.08-7.96 (m, 2H), 7.75- 7.51 (m, 3H), 7.51-7.40 (m, 2H), 7.19 (s, 1H), 7.09 (d, J = 8.2 Hz, 1H), 4.66 (t, J = 7.9 Hz, 2H), 3.21 (t, J = 7.8 Hz, 2H), 2.33 (s, 3H), 2.08 (d, J = 6.8 Hz, 3H). |
| 86 | 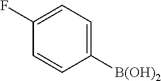 | 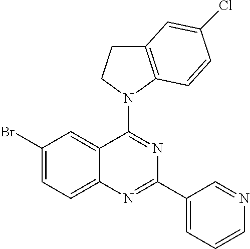 [SMILES: Clc1ccc2c(c1)CCN2c1nc(-c2cccnc2)nc2ccc(Br)cc12] | 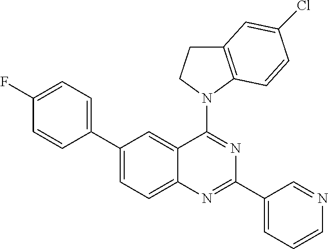 [SMILES: Fc1ccc(-c2ccc3nc(-c4cccnc4)nc(N4CCc5cc(Cl)ccc54)c3c2)cc1] | 2HCl | 1H NMR (400 MHz, DMSO) δ 9.59 (d, J = 1.8 Hz, 1H), 9.17 (d, J = 8.1 Hz, 1H), 8.98 (dd, J = 5.4, 1.4 Hz, 1H), 8.43 (d, J = 1.6 Hz, 1H), 8.31 (dd, J = 8.8, 1.8 Hz, 1H), 8.14 (d, J = 8.7 Hz, 1H), 8.05 (dd, J = 8.0, 5.5 Hz, 1H), 7.99-7.84 (m, 3H), 7.49 (d, J = 2.1 Hz, 1H), 7.37 (dd, J = 14.6, 5.7 Hz, 3H), 4.80 (t, J = 7.8 Hz, 2H), 3.27 (t, J = 7.6 Hz, 2H). |
| 87 | 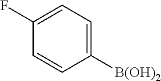 | 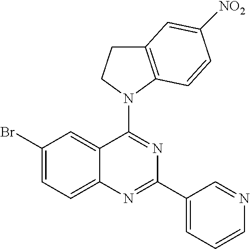 [SMILES: O=[N+]([O-])c1ccc2c(c1)CCN2c1nc(-c2cccnc2)nc2ccc(Br)cc12] | 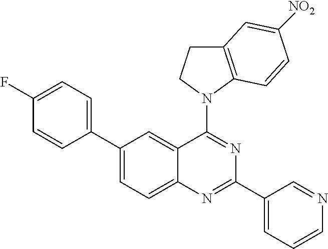 [SMILES: O=[N+]([O-])c1ccc2c(c1)CCN2c1nc(-c2cccnc2)nc2ccc(-c3ccc(F)cc3)cc12] | | 1H NMR (400 MHz, DMSO) δ 9.59 (d, J = 1.3 Hz, 1H), 8.79-8.67 (m, 2H), 8.42-8.27 (m, 2H), 8.27-8.16 (m, 2H), 8.11 (d, J = 8.7 Hz, 1H), 7.92-7.82 (m, 2H), 7.76 (d, J = 8.9 Hz, 1H), 7.59 (dd, J = 7.9, 4.8 Hz, 1H), 7.36 (t, J = 8.8 Hz, 2H), 4.78 (t, J = 8.2 Hz, 2H), 3.36 (t, J = 8.3 Hz, 2H). |
| 88 | 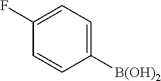 | 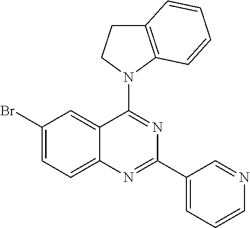 [SMILES: Brc1ccc2nc(-c3cccnc3)nc(N3CCc4ccccc43)c2c1] | 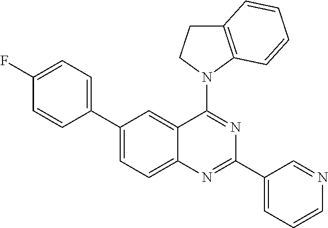 [SMILES: Fc1ccc(-c2ccc3nc(-c4cccnc4)nc(N4CCc5ccccc54)c3c2)cc1] | 2HCl | 1H NMR (400 MHz, DMSO) δ 9.59 (d, J = 1.5 Hz, 1H), 9.02 (d, J = 8.1 Hz, 1H), 8.88 (dd, J = 5.2, 1.5 Hz, 1H), 8.43 (d, J = 1.8 Hz, 1H), 8.28 (dd, J = 8.8, 2.0 Hz, 1H), 8.10 (d, J = 8.7 Hz, 1H), 7.97-7.82 (m, 4H), 7.49-7.26 (m, 4H), 7.12 (t, J = 7.2 Hz, 1H), 4.75 (t, J = 7.8 Hz, 2H), 3.26 (t, J = 7.8 Hz, 2H). |
| 89 | 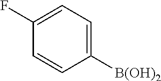 | 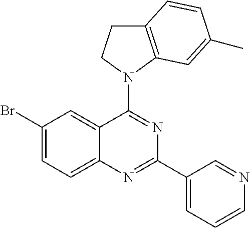 [SMILES: Cc1ccc2c(c1)N(c1nc(-c3cccnc3)nc3ccc(Br)cc13)CC2] | 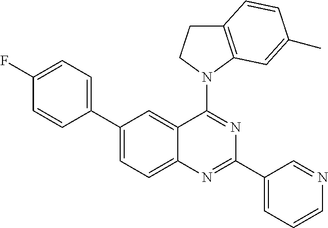 [SMILES: Cc1ccc2c(c1)N(c1nc(-c3cccnc3)nc3ccc(-c4ccc(F)cc4)cc13)CC2] | 2HCl | 1H NMR (400 MHz, DMSO) δ 9.56 (s, 1H), 9.14 (d, J = 8.1 Hz, 1H), 8.96 (d, J = 5.2 Hz, 1H), 8.41 (s, 1H), 8.28 (d, J = 8.8 Hz, 1H), 8.13 (d, J = 8.7 Hz, 1H), 8.01 (dd, J = 7.9, 5.4 Hz, 1H), 7.85 (dd, J = 8.7, 5.4 Hz, 2H), 7.74 (s, 1H), 7.37 (t, J = 8.8 Hz, 2H), 7.29 (d, J = 7.6 Hz, 1H), 6.96 (d, J = 7.4 Hz, 1H), 4.74 (t, J = 7.6 Hz, 2H), 3.20 (t, J = 7.5 Hz, 2H), 2.38 (s, 3H). |
| 90 | 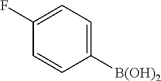 | 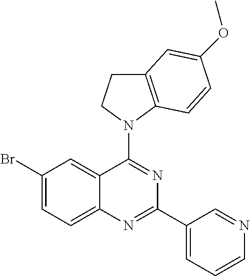 [SMILES: COc1ccc2c(c1)CCN2c1nc(-c2cccnc2)nc2ccc(Br)cc12] | 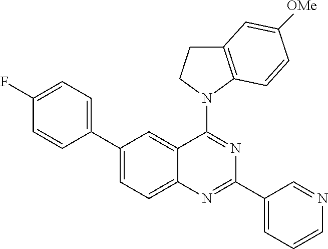 [SMILES: COc1ccc2c(c1)CCN2c1nc(-c2cccnc2)nc2ccc(-c3ccc(F)cc3)cc12] | 2HCl | 1H NMR (400 MHz, DMSO) δ 9.57 (d, J = 1.6 Hz, 1H), 9.04 (d, J = 8.0 Hz, 1H), 8.94 (dd, J = 5.2, 1.5 Hz, 1H), 8.48 (s, 1H), 8.36-8.25 (m, 1H), 8.15 (d, J = 9.0 Hz, 1H), 8.03 (d, J = 8.2 Hz, 1H), 7.99-7.83 (m, 3H), 7.39 (t, J = 8.9 Hz, 2H), 7.06 (d, J = 2.5 Hz, 1H), 6.94 (dd, J = 8.8, 2.6 Hz, 1H), 4.84 (t, J = 7.6 Hz, 2H), 3.82 (s, 3H), 3.25 (t, J = 7.5 Hz, 2H). |
| 91 | 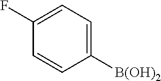 | 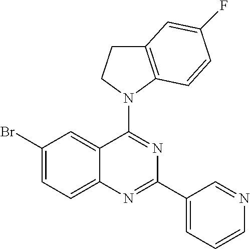 [SMILES: Fc1ccc2c(c1)CCN2c1nc(-c2cccnc2)nc2ccc(Br)cc12] | 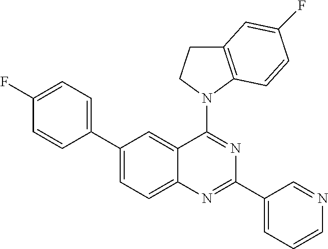 [SMILES: Fc1ccc(-c2ccc3nc(-c4cccnc4)nc(N4CCc5cc(F)ccc54)c3c2)cc1] | 2HCl | 1H NMR (400 MHz, DMSO) δ 9.57 (s, 1H), 9.09 (d, J = 8.3 Hz, 1H), 8.94 (d, J = 5.0 Hz, 1H), 8.42 (s, 1H), 8.29 (d, J = 8.8 Hz, 1H), 8.13 (d, J = 8.7 Hz, 1H), 8.04-7.93 (m, 2H), 7.93-7.82 (m, 2H), 7.38 (t, J = 8.8 Hz, 2H), 7.30 (dd, J = 8.3, 2.5 Hz, 1H), 7.23- 7.09 (m, 1H), 4.81 (t, J = 7.8 Hz, 2H), 3.27 (t, J = 7.7 Hz, 2H). |
| 92 | 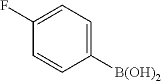 | 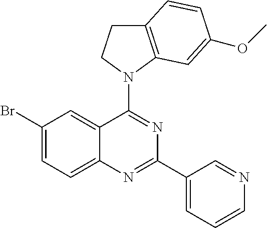 [SMILES: COc1ccc2c(c1)N(c1nc(-c3cccnc3)nc3ccc(Br)cc13)CC2] | 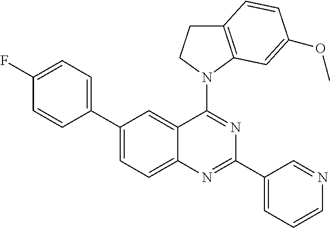 [SMILES: COc1ccc2c(c1)N(c1nc(-c3cccnc3)nc3ccc(-c4ccc(F)cc4)cc13)CC2] | 2HCl | 1H NMR (400 MHz, DMSO) δ 9.60 (d, J = 1.8 Hz, 1H), 9.19 (d, J = 8.0 Hz, 1H), 8.98 (dd, J = 5.3, 1.4 Hz, 1H), 8.46 (d, J = 1.7 Hz, 1H), 8.31 (dd, J = 8.8, 1.9 Hz, 1H), 8.17 (d, J = 8.8 Hz, 1H), 8.04 (dd, J = 8.0, 5.4 Hz, 1H), 7.95-7.84 (m, 2H), 7.52 (s, 1H), 7.44- 7.34 (m, 2H), 7.31 (d, J = 8.3 Hz, 1H), 6.73 (dd, J = 8.3, 2.3 Hz, 1H), 4.80 (t, J = 7.7 Hz, 2H), 3.75 (s, 3H), 3.18 (t, J = 7.7 Hz, 2H). |
| 93 | 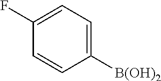 | 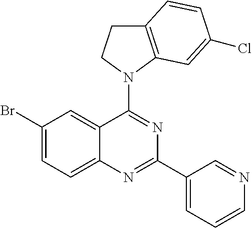 [SMILES: Clc1ccc2c(c1)N(c1nc(-c3cccnc3)nc3ccc(Br)cc13)CC2] | 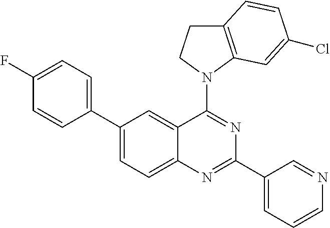 [SMILES: Fc1ccc(-c2ccc3nc(-c4cccnc4)nc(N4CCc5ccc(Cl)cc54)c3c2)cc1] | 2HCl | 1H NMR (400 MHz, DMSO) δ 9.57 (d, J = 1.6 Hz, 1H), 9.13 (d, J = 8.3 Hz, 1H), 8.96 (d, J = 5.3 Hz, 1H), 8.42 (d, J = 1.6 Hz, 1H), 8.31 (dd, J = 8.8, 1.8 Hz, 1H), 8.13 (d, J = 8.7 Hz, 1H), 8.02 (dd, J = 8.1, 5.4 Hz, 1H), 7.97- 7.83 (m, 3H), 7.49-7.32 (m, 3H), 7.16 (dd, J = 8.0, 1.9 Hz, 1H), 4.79 (t, J = 7.9 Hz, 2H), 3.25 (t, J = 7.9 Hz, 2H). |
| 94 | 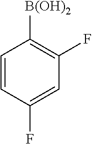 [SMILES: OB(O)c1ccc(F)cc1F] | 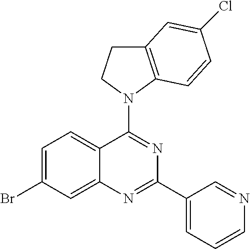 [SMILES: Clc1ccc2c(c1)CCN2c1nc(-c2cccnc2)nc2cc(Br)ccc12] | 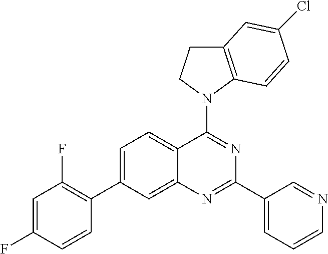 [SMILES: Fc1ccc(-c2ccc3c(N4CCc5cc(Cl)ccc54)nc(-c4cccnc4)nc3c2)c(F)c1] | 2HCl | 1H NMR (400 MHz, DMSO) δ 9.60 (d, J = 1.8 Hz, 1H), 9.19 (d, J = 8.2 Hz, 1H), 8.99 (dd, J = 5.4, 1.4 Hz, 1H), 8.40 (d, J = 8.8 Hz, 1H), 8.20 (s, 1H), 8.06 (dd, J = 8.1, 5.4 Hz, 1H), 7.96 (d, J = 8.7 Hz, 1H), 7.91-7.79 (m, 2H), 7.58-7.45 (m, 2H), 7.42-7.26 (m, 2H), 4.73 (t, J = 7.9 Hz, 2H), 3.27 (t, J = 7.8 Hz, 2H). |
| 95 | 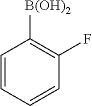 | 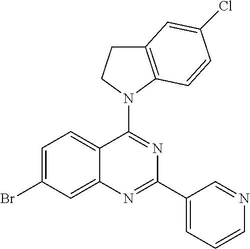 [SMILES: Clc1ccc2c(c1)CCN2c1nc(-c2cccnc2)nc2cc(Br)ccc12] | 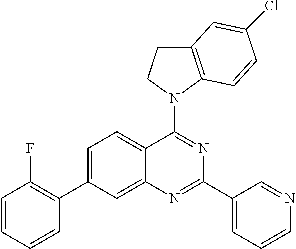 [SMILES: Fc1ccccc1-c1ccc2c(N3CCc4cc(Cl)ccc43)nc(-c3cccnc3)nc2c1] | 2HCl | 1H NMR (400 MHz, DMSO) δ 9.60-9.54 (m, 1H), 8.76-8.67 (m, 2H), 8.31 (d, J = 8.8 Hz, 1H), 8.13-8.06 (m, 1H), 7.84 (d, J = 8.6 Hz, 1H), 7.81-7.71 (m, 2H), 7.60-7.50 (m, 2H), 7.47-7.30 (m, 4H), 4.66 (t, J = 8.0 Hz, 2H), 3.25 (t, J = 7.9 Hz, 2H). |
| 96 | 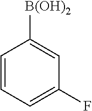 | 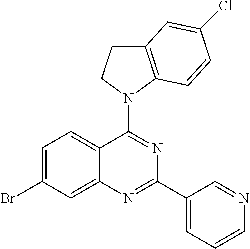 [SMILES: Clc1ccc2c(c1)CCN2c1nc(-c2cccnc2)nc2cc(Br)ccc12] | 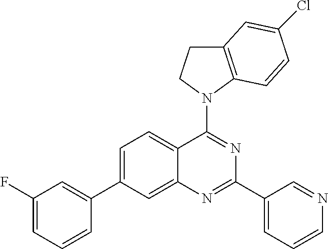 [SMILES: Fc1cccc(-c2ccc3c(N4CCc5cc(Cl)ccc54)nc(-c4cccnc4)nc3c2)c1] | | 1H NMR (400 MHz, DMSO) δ 9.63-9.55 (m, 1H), 8.76-8.65 (m, 2H), 8.34-8.21 (m, 2H), 7.94 (dd, J = 8.8, 2.0 Hz, 1H), 7.86- 7.74 (m, 3H), 7.67-7.54 (m, 2H), 7.44 (d, J = 2.1 Hz, 1H), 7.38-7.28 (m, 2H), 4.65 (t, J = 8.0 Hz, 2H), 3.25 (t, J = 7.8 Hz, 2H). |
| 97 | 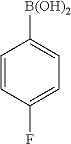 | 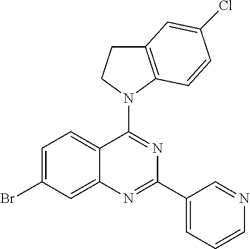 [SMILES: Clc1ccc2c(c1)CCN2c1nc(-c2cccnc2)nc2cc(Br)ccc12] | 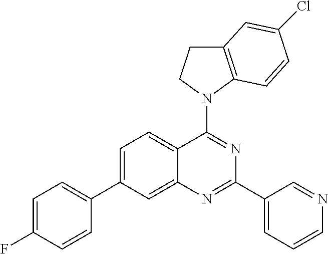 [SMILES: Fc1ccc(-c2ccc3c(N4CCc5cc(Cl)ccc54)nc(-c4cccnc4)nc3c2)cc1] | | 1H NMR (400 MHz, DMSO) δ 9.62-9.55 (m, 1H), 8.77-8.67 (m, 2H), 8.29 (d, J = 8.8 Hz, 1H), 8.20 (d, J = 1.9 Hz, 1H), 8.04- 7.95 (m, 2H), 7.91 (dd, J = 8.8, 2.0 Hz, 1H), 7.81 (d, J = 8.6 Hz, 1H), 7.63-7.54 (m, 1H), 7.45 (d, J = 2.2 Hz, 1H), 7.44-7.37 (m, 2H), 7.34 (dd, J = 8.6, 2.3 Hz, 1H), 4.66 (t, J = 8.0 Hz, 2H), 3.26 (t, J = 8.0 Hz, 2H). |
| 98 | 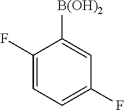 [SMILES: OB(O)c1cc(F)ccc1F] | 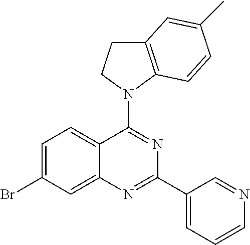 [SMILES: Cc1ccc2c(c1)CCN2c1nc(-c2cccnc2)nc2cc(Br)ccc12] | 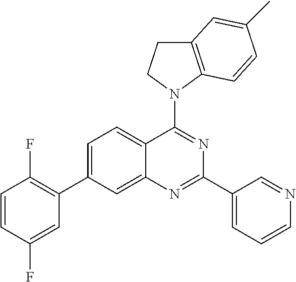 [SMILES: Cc1ccc2c(c1)CCN2c1nc(-c2cccnc2)nc2cc(-c3cc(F)ccc3F)ccc12] | | 1H NMR (400 MHz, DMSO) δ 9.63-9.54 (m, 1H), 8.77-8.65 (m, 2H), 8.32 (d, J = 8.8 Hz, 1H), 8.11 (s, 1H), 7.76 (d, J = 8.0 Hz, 2H), 7.71-7.63 (m, 1H), 7.62-7.54 (m, 1H), 7.54-7.32 (m, 2H), 7.20 (s, 1H), 7.11 (d, J = 8.2 Hz, 1H), 4.63 (t, J = 7.9 Hz, 2H), 3.20 (t, J = 7.8 Hz, 2H), 2.34 (s, 3H). |
| 99 | 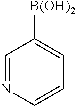 | 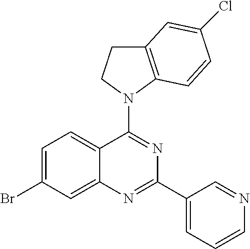 [SMILES: Clc1ccc2c(c1)CCN2c1nc(-c2cccnc2)nc2cc(Br)ccc12] | 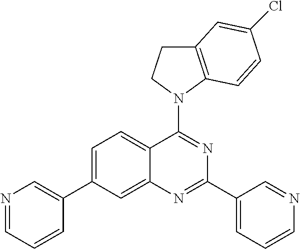 [SMILES: Clc1ccc2c(c1)CCN2c1nc(-c2cccnc2)nc2cc(-c3cccnc3)ccc12] | | 1H NMR (400 MHz, DMSO) δ 9.59 (d, J = 2.1 Hz, 1H), 9.14 (d, J = 1.7 Hz, 1H), 8.76- 8.66 (m, 3H), 8.39-8.29 (m, 3H), 7.98 (dd, J = 8.8, 2.0 Hz, 1H), 7.89-7.80 (m, 1H), 7.64-7.56 (m, 2H), 7.45 (d, J = 2.1 Hz, 1H), 7.35 (dd, J = 8.6, 2.3 Hz, 1H), 4.67 (t, J = 8.0 Hz, 2H), 3.26 (t, J = 8.1 Hz, 2H). |
| 100 | 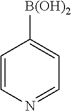 | 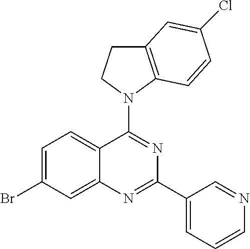 [SMILES: Clc1ccc2c(c1)CCN2c1nc(-c2cccnc2)nc2cc(Br)ccc12] | 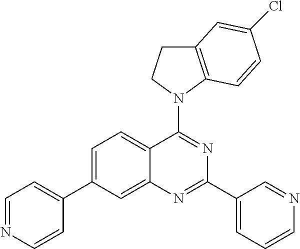 [SMILES: Clc1ccc2c(c1)CCN2c1nc(-c2cccnc2)nc2cc(-c3ccncc3)ccc12] | | 1H NMR (400 MHz, DMSO) δ 9.59 (d, J = 2.0 Hz, 1H), 8.82-8.69 (m, 4H), 8.43-8.32 (m, 2H), 8.05-7.92 (m, 3H), 7.89-7.80 (m, 1H), 7.64-7.55 (m, 1H), 7.46 (d, J = 2.1 Hz, 1H), 7.35 (dd, J = 8.6, 2.3 Hz, 1H), 4.67 (t, J = 8.0 Hz, 2H), 3.27 (t, J = 7.9 Hz, 2H). |
| 101 | 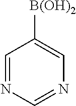 | 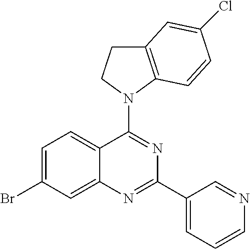 [SMILES: Clc1ccc2c(c1)CCN2c1nc(-c2cccnc2)nc2cc(Br)ccc12] | 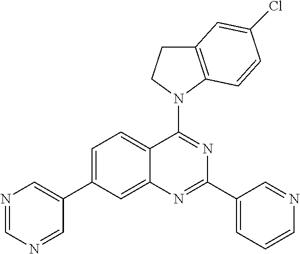 [SMILES: Clc1ccc2c(c1)CCN2c1nc(-c2cccnc2)nc2cc(-c3cncnc3)ccc12] | | 1H NMR (400 MHz, DMSO) δ 9.57 (s, 1H), 9.39 (s, 2H), 9.30 (s, 1H), 8.75-8.65 (m, 2H), 8.40 (d, J = 1.7 Hz, 1H), 8.34 (d, J = 8.8 Hz, 1H), 8.03 (dd, J = 8.7, 1.9 Hz, 1H), 7.84 (d, J = 8.6 Hz, 1H), 7.58 (dd, J = 7.5, 5.2 Hz, 1H), 7.45 (d, J = 2.0 Hz, 1H), 7.34 (dd, J = 8.6, 2.2 Hz, 1H), 4.66 (t, J = 8.0 Hz, 2H), 3.26 (t, J = 8.0 Hz, 2H). |
| 102 | 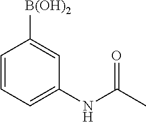 | 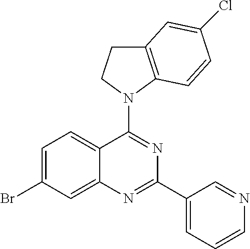 [SMILES: Clc1ccc2c(c1)CCN2c1nc(-c2cccnc2)nc2cc(Br)ccc12] | 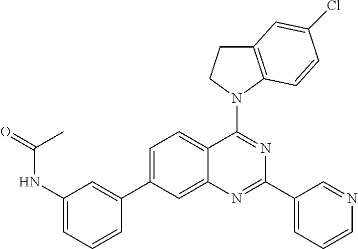 [SMILES: CC(=O)Nc1cccc(-c2ccc3c(N4CCc5cc(Cl)ccc54)nc(-c4cccnc4)nc3c2)c1] | 2HCl | 1H NMR (400 MHz, DMSO) δ 10.27 (s, 1H), 9.61 (d, J = 1.7 Hz, 1H), 9.19 (d, J = 8.1 Hz, 1H), 8.99 (d, J = 5.2 Hz, 1H), 8.40 (d, J = 8.9 Hz, 1H), 8.25 (s, 2H), 8.06 (dd, J = 8.0, 5.4 Hz, 1H), 7.94 (dd, J = 12.6, 5.3 Hz, 2H), 7.65 (d, J = 8.0 Hz, 1H), 7.61-7.45 (m, 3H), 7.36 (dd, J = 8.6, 2.2 Hz, 1H), 4.73 (t, J = 7.9 Hz, 2H), 3.28 (t, J = 7.8 Hz, 2H), 2.10 (d, J = 9.4 Hz, 3H). |
| 103 | 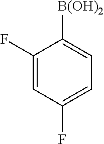 [SMILES: OB(O)c1ccc(F)cc1F] | 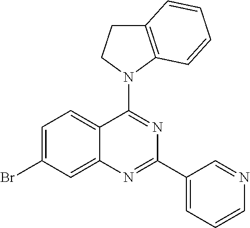 [SMILES: Brc1ccc2c(N3CCc4ccccc43)nc(-c3cccnc3)nc2c1] | 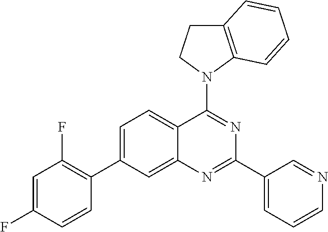 [SMILES: Fc1ccc(-c2ccc3c(N4CCc5ccccc54)nc(-c4cccnc4)nc3c2)c(F)c1] | | 1H NMR (400 MHz, CDCl3) δ 9.75 (d, J = 1.6 Hz, 1H), 8.84-8.77 (m, 1H), 8.70 (dd, J = 4.8, 1.7 Hz, 1H), 8.13 (d, J = 8.9 Hz, 2H), 7.67-7.55 (m, 2H), 7.49-7.37 (m, 2H), 7.33 (d, J = 7.3 Hz, 1H), 7.26 (s, 1H), 7.19 (t, J = 7.8 Hz, 1H), 7.09-6.95 (m, 3H), 4.56 (t, J = 8.0 Hz, 2H), 3.28 (t, J = 7.9 Hz, 2H). |
| 104 | 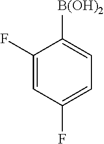 [SMILES: OB(O)c1ccc(F)cc1F] | 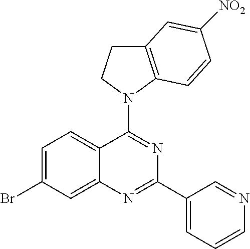 [SMILES: O=[N+]([O-])c1ccc2c(c1)CCN2c1nc(-c2cccnc2)nc2cc(Br)ccc12] | 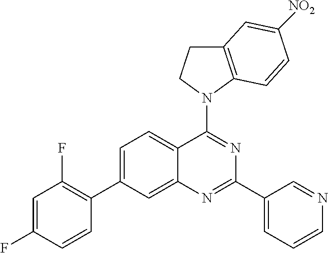 [SMILES: O=[N+]([O-])c1ccc2c(c1)CCN2c1nc(-c2cccnc2)nc2cc(-c3ccc(F)cc3F)ccc12] | | 1H NMR (400 MHz, CDCl3) δ 9.76 (d, J = 1.4 Hz, 1H), 8.87-8.78 (m, 1H), 8.74 (dd, J = 4.8, 1.7 Hz, 1H), 8.30-8.16 (m, 2H), 8.11 (dd, J = 8.9, 2.4 Hz, 1H), 8.04 (d, J = 8.7 Hz, 1H), 7.77-7.67 (m, 1H), 7.67-7.56 (m, 1H), 7.51-7.41 (m, 1H), 7.31-7.24 (m, 1H), 7.13-6.96 (m, 2H), 4.68 (t, J = 8.2 Hz, 2H), 3.39 (t, J = 8.2 Hz, 2H). |
| 105 | 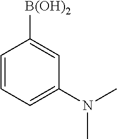 | 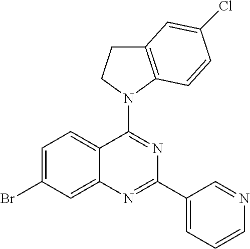 [SMILES: Clc1ccc2c(c1)CCN2c1nc(-c2cccnc2)nc2cc(Br)ccc12] | 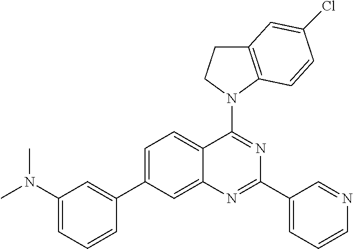 [SMILES: CN(C)c1cccc(-c2ccc3c(N4CCc5cc(Cl)ccc54)nc(-c4cccnc4)nc3c2)c1] | 3HCl | 1H NMR (400 MHz, DMSO) δ 9.61 (d, J = 1.7 Hz, 1H), 9.22 (d, J = 8.2 Hz, 1H), 9.01 (d, J = 4.2 Hz, 1H), 8.39 (d, J = 9.1 Hz, 2H), 8.18- 7.92 (m, 3H), 7.92-7.56 (m, 3H), 7.49 (d, J = 2.1 Hz, 2H), 7.36 (dd, J = 8.6, 2.2 Hz, 1H), 4.73 (t, J = 7.9 Hz, 2H), 3.28 (t, J = 7.7 Hz, 2H), 3.15 (s, 6H). |
| 106 | 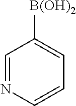 | 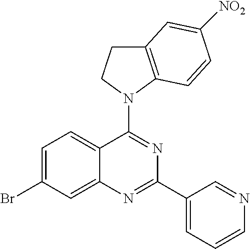 [SMILES: O=[N+]([O-])c1ccc2c(c1)CCN2c1nc(-c2cccnc2)nc2cc(Br)ccc12] | 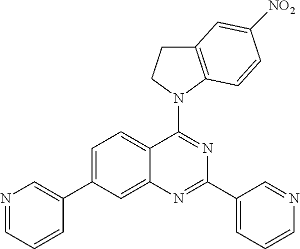 [SMILES: O=[N+]([O-])c1ccc2c(c1)CCN2c1nc(-c2cccnc2)nc2cc(-c3cccnc3)ccc12] | | 1H NMR (400 MHz, DMSO) δ 9.62 (s, 1H), 9.16 (s, 1H), 8.83-8.65 (m, 3H), 8.45- 8.30 (m, 3H), 8.30-8.17 (m, 2H), 8.05 (d, J = 8.7 Hz, 1H), 7.78 (d, J = 8.8 Hz, 1H), 7.60 (dd, J = 7.8, 4.7 Hz, 2H), 4.75 (t, J = 8.2 Hz, 2H), 3.38 (t, J = 7.8 Hz, 2H). |
| 107 | 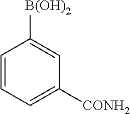 [SMILES: NC(=O)c1cccc(B(O)O)c1] | 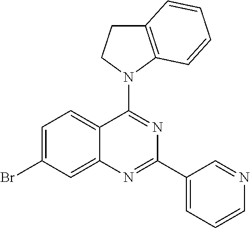 [SMILES: Brc1ccc2c(N3CCc4ccccc43)nc(-c3cccnc3)nc2c1] | 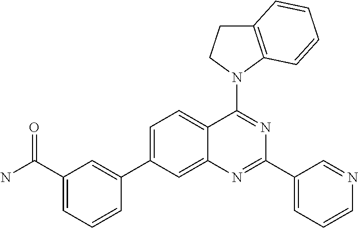 [SMILES: NC(=O)c1cccc(-c2ccc3c(N4CCc5ccccc54)nc(-c4cccnc4)nc3c2)c1] | | 1H NMR (400 MHz, DMSO) δ 9.61 (dd, J = 2.1, 0.7 Hz, 1H), 8.78-8.68 (m, 2H), 8.43 (s, 1H), 8.37-8.29 (m, 2H), 8.26 (s, 1H), 8.09 (d, J = 7.8 Hz, 1H), 8.03-7.93 (m, 2H), 7.78 (d, J = 8.0 Hz, 1H), 7.65 (t, J = 7.7 Hz, 1H), 7.62-7.54 (m, 1H), 7.52 (s, 1H), 7.39 (d, J = 7.3 Hz, 1H), 7.28 (t, J = 7.3 Hz, 1H), 7.07 (td, J = 7.4, 0.8 Hz, 1H), 4.63 (t, J = 8.0 Hz, 2H), 3.25 (t, J = 7.8 Hz, 2H). |
| 108 | 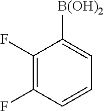 [SMILES: OB(O)c1cccc(F)c1F] | 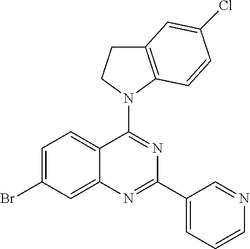 [SMILES: Clc1ccc2c(c1)CCN2c1nc(-c2cccnc2)nc2cc(Br)ccc12] | 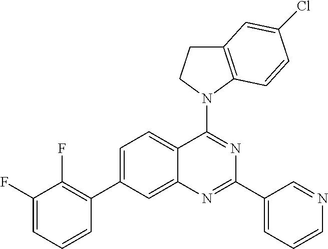 [SMILES: Fc1cccc(-c2ccc3c(N4CCc5cc(Cl)ccc54)nc(-c4cccnc4)nc3c2)c1F] | | 1H NMR (400 MHz, DMSO) δ 9.59 (d, J = 1.7 Hz, 1H), 9.10 (d, J = 8.1 Hz, 1H), 8.93 (dd, J = 5.3, 1.4 Hz, 1H), 8.41 (d, J = 8.8 Hz, 1H), 8.20 (s, 1H), 8.03-7.91 (m, 2H), 7.86 (d, J = 8.8 Hz, 1H), 7.70-7.53 (m, 2H), 7.53- 7.33 (m, 3H), 4.72 (t, J = 7.9 Hz, 2H), 3.27 (t, J = 7.7 Hz, 2H). |
| 109 | 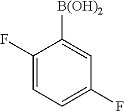 [SMILES: OB(O)c1cc(F)ccc1F] | 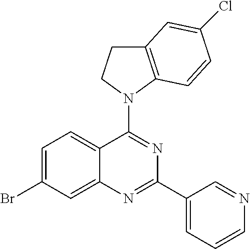 [SMILES: Clc1ccc2c(c1)CCN2c1nc(-c2cccnc2)nc2cc(Br)ccc12] | 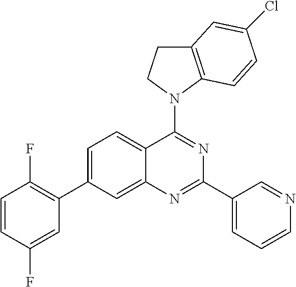 [SMILES: Fc1ccc(F)c(-c2ccc3c(N4CCc5cc(Cl)ccc54)nc(-c4cccnc4)nc3c2)c1] | HCl | 1H NMR (400 MHz, DMSO) δ 9.58 (d, J = 1.5 Hz, 1H), 9.09 (d, J = 8.2 Hz, 1H), 8.93 (dd, J = 5.3, 1.5 Hz, 1H), 8.38 (d, J = 8.8 Hz, 1H), 8.21 (s, 1H), 8.06-7.90 (m, 2H), 7.85 (dd, J = 7.0, 1.7 Hz, 1H), 7.77-7.61 (m, 1H), 7.59- 7.32 (m, 4H), 4.71 (t, J = 8.0 Hz, 3H), 3.27 (t, J = 7.8 Hz, 2H). |
| 110 | 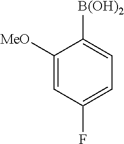 [SMILES: COc1cc(F)ccc1B(O)O] | 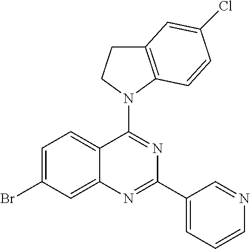 [SMILES: Clc1ccc2c(c1)CCN2c1nc(-c2cccnc2)nc2cc(Br)ccc12] | 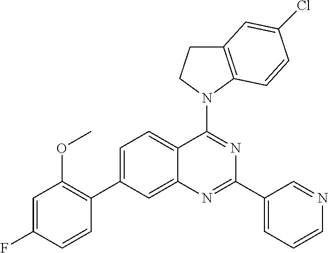 [SMILES: COc1cc(F)ccc1-c1ccc2c(N3CCc4cc(Cl)ccc43)nc(-c3cccnc3)nc2c1] | 2HCl | 1H NMR (400 MHz, DMSO) δ 9.59 (d, J = 1.8 Hz, 1H), 9.18 (d, J = 8.2 Hz, 1H), 8.99 (dd, J = 5.4, 1.4 Hz, 1H), 8.33 (d, J = 8.8 Hz, 1H), 8.18 (s, 1H), 8.05 (dd, J = 8.0, 5.5 Hz, 1H), 7.98 (d, J = 8.6 Hz, 1H), 7.80 (dd, J = 8.8, 1.8 Hz, 1H), 7.55 (dd, J = 8.5, 6.9 Hz, 1H), 7.49 (d, J = 2.1 Hz, 1H), 7.37 (dd, J = 8.6, 2.2 Hz, 1H), 7.16 (dd, J = 11.5, 2.5 Hz, 1H), 6.97 (td, J = 8.4, 2.5 Hz, 1H), 4.73 (t, J = 7.9 Hz, 2H), 3.87 (s, 3H), 3.27 (t, J = 7.8 Hz, 2H). |
| 111 | 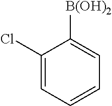 | 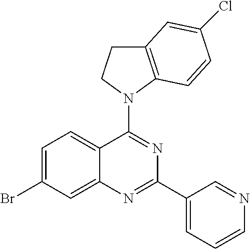 [SMILES: Clc1ccc2c(c1)CCN2c1nc(-c2cccnc2)nc2cc(Br)ccc12] | 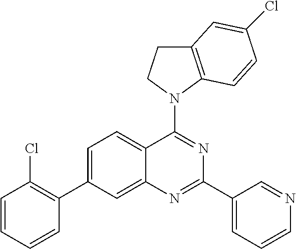 [SMILES: Clc1ccc2c(c1)CCN2c1nc(-c2cccnc2)nc2cc(-c3ccccc3Cl)ccc12] | HCl | 1H NMR (400 MHz, DMSO) δ 9.58 (d, J = 1.5 Hz, 1H), 9.00 (d, J = 8.3 Hz, 1H), 8.87 (dd, J = 5.1, 1.5 Hz, 1H), 8.38 (d, J = 8.7 Hz, 1H), 8.03 (d, J = 1.8 Hz, 1H), 7.94 (d, J = 8.6 Hz, 1H), 7.87 (dd, J = 7.9, 5.0 Hz, 1H), 7.76- 7.65 (m, 2H), 7.63-7.58 (m, 1H), 7.58- 7.45 (m, 3H), 7.37 (dd, J = 8.6, 2.3 Hz, 1H), 4.72 (t, J = 8.0 Hz, 2H), 3.27 (t, J = 7.8 Hz, 2H). |
| 112 | 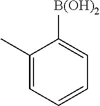 | 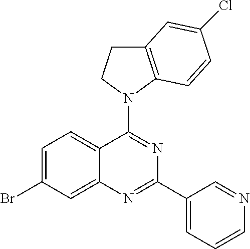 [SMILES: Clc1ccc2c(c1)CCN2c1nc(-c2cccnc2)nc2cc(Br)ccc12] | 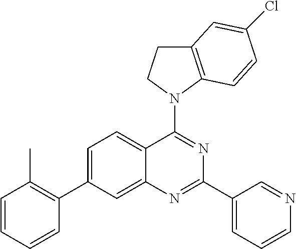 [SMILES: Cc1ccccc1-c1ccc2c(N3CCc4cc(Cl)ccc43)nc(-c3cccnc3)nc2c1] | 2HCl | 1H NMR (400 MHz, DMSO) δ 9.61 (d, J = 1.8 Hz, 1H), 9.23 (d, J = 8.2 Hz, 1H), 9.01 (dd, J = 5.4, 1.3 Hz, 1H), 8.40 (d, J = 8.7 Hz, 1H), 8.16-8.05 (m, 2H), 8.01 (d, J = 8.6 Hz, 1H), 7.71 (dd, J = 8.7, 1.8 Hz, 1H), 7.50 (d, J = 2.0 Hz, 1H), 7.47-7.29 (m, 5H), 4.77 (t, J = 7.8 Hz, 2H), 3.28 (t, J = 7.8 Hz, 2H), 2.35 (s, 3H). |
| 113 | 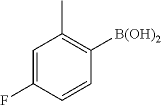 | 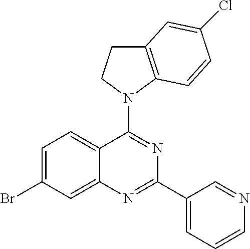 [SMILES: Clc1ccc2c(c1)CCN2c1nc(-c2cccnc2)nc2cc(Br)ccc12] | 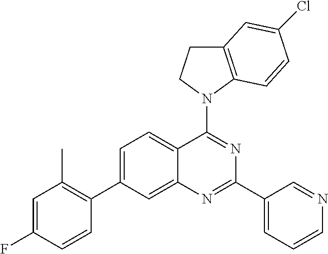 [SMILES: Cc1cc(F)ccc1-c1ccc2c(N3CCc4cc(Cl)ccc43)nc(-c3cccnc3)nc2c1] | 2HCl | 1H NMR (400 MHz, DMSO) δ 9.59 (d, J = 1.8 Hz, 1H), 9.18 (d, J = 8.2 Hz, 1H), 8.98 (dd, J = 5.4, 1.4 Hz, 1H), 8.38 (d, J = 8.7 Hz, 1H), 8.09-7.93 (m, 3H), 7.68 (dd, J = 8.7, 1.8 Hz, 1H), 7.54-7.35 (m, 3H), 7.31-7.14 (m, 2H), 4.75 (t, J = 7.9 Hz, 2H), 3.28 (t, J = 7.8 Hz, 2H), 2.35 (s, 3H). |
| 114 | 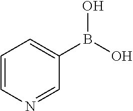 | 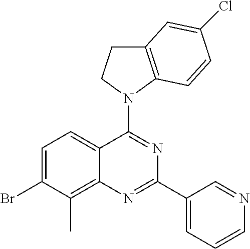 [SMILES: Cc1c(Br)ccc2c(N3CCc4cc(Cl)ccc43)nc(-c3cccnc3)nc12] | 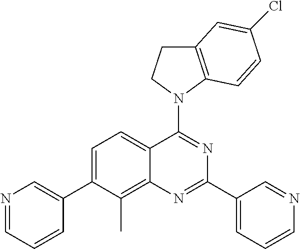 [SMILES: Cc1c(-c2cccnc2)ccc2c(N3CCc4cc(Cl)ccc43)nc(-c3cccnc3)nc12] | 2HCl | 1H NMR (400 MHz, DMSO) δ 9.64 (d, J = 1.6 Hz, 1H), 9.12 (d, J = 8.2 Hz, 1H), 8.98- 8.81 (m, 3H), 8.36 (d, J = 8.1 Hz, 1H), 8.20 (d, J = 8.7 Hz, 1H), 7.99-7.84 (m, 2H), 7.79 (d, J = 8.6 Hz, 1H), 7.59 (d, J = 8.7 Hz, 1H), 7.47 (d, J = 2.2 Hz, 1H), 7.33 (dd, J = 8.6, 2.3 Hz, 1H), 4.66 (t, J = 8.0 Hz, 2H), 3.26 (t, J = 7.9 Hz, 2H), 2.74 (s, 3H). |
| 115 | 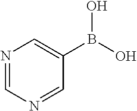 | 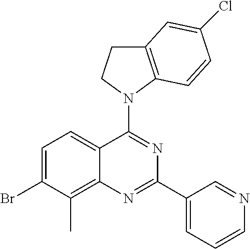 [SMILES: Cc1c(Br)ccc2c(N3CCc4cc(Cl)ccc43)nc(-c3cccnc3)nc12] | 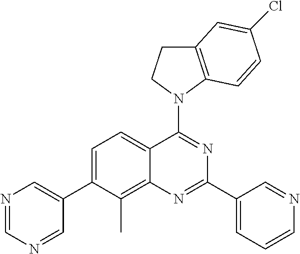 [SMILES: Cc1c(-c2cncnc2)ccc2c(N3CCc4cc(Cl)ccc43)nc(-c3cccnc3)nc12] | 2 HCl | 1H NMR (400 MHz, DMSO) δ 9.64 (d, J = 1.8 Hz, 1H), 9.43-9.22 (m, 2H), 9.10-8.95 (m, 3H), 8.21 (d, J = 8.7 Hz, 1H), 8.10 (dd, J = 8.0, 5.5 Hz, 1H), 7.82 (d, J = 8.6 Hz, 1H), 7.63 (d, J = 8.7 Hz, 1H), 7.47 (d, J = 2.1 Hz, 1H), 7.33 (dd, J = 8.6, 2.3 Hz, 1H), 4.67 (t, J = 8.0 Hz, 2H), 3.26 (t, J = 7.8 Hz, 2H), 2.76 (s, 3H). |
| 116 | 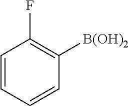 | 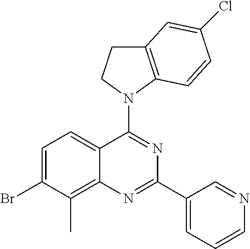 [SMILES: Cc1c(Br)ccc2c(N3CCc4cc(Cl)ccc43)nc(-c3cccnc3)nc12] | 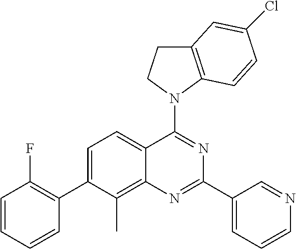 [SMILES: Cc1c(-c2ccccc2F)ccc2c(N3CCc4cc(Cl)ccc43)nc(-c3cccnc3)nc12] | HCl | 1H NMR (400 MHz, DMSO) δ 9.63 (d, J = 1.7 Hz, 1H), 9.18 (d, J = 8.1 Hz, 1H), 8.94 (dd, J = 5.3, 1.4 Hz, 1H), 8.16 (d, J = 8.7 Hz, 1H), 8.00 (dd, J = 8.0, 5.4 Hz, 1H), 7.80 (d, J = 8.6 Hz, 1H), 7.64-7.37 (m, 6H), 7.32 (dd, J = 8.6, 2.3 Hz, 1H), 4.65 (t, J = 8.0 Hz, 2H), 3.25 (t, J = 7.9 Hz, 2H), 2.62 (s, 3H). |
| 117 | 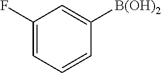 | 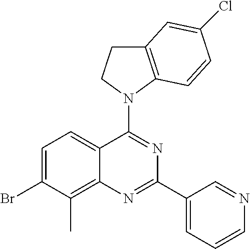 [SMILES: Cc1c(Br)ccc2c(N3CCc4cc(Cl)ccc43)nc(-c3cccnc3)nc12] | 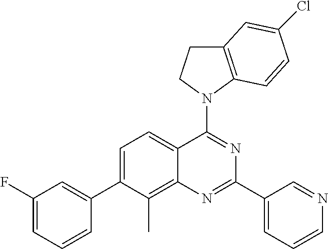 [SMILES: Cc1c(-c2cccc(F)c2)ccc2c(N3CCc4cc(Cl)ccc43)nc(-c3cccnc3)nc12] | HCl | 1H NMR (400 MHz, DMSO) δ 9.63 (s, 1H), 9.15 (d, J = 8.3 Hz, 1H), 8.92 (d, J = 4.3 Hz, 1H), 8.14 (d, J = 8.8 Hz, 1H), 7.98 (dd, J = 7.9, 5.2 Hz, 1H), 7.77 (d, J = 8.6 Hz, 1H), 7.65-7.44 (m, 3H), 7.41-7.27 (m, 4H), 4.64 (t, J = 8.0 Hz, 2H), 3.25 (t, J = 7.8 Hz, 2H), 2.72 (s, 3H). |
| 118 | 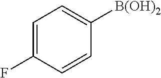 | 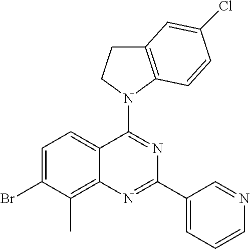 [SMILES: Cc1c(Br)ccc2c(N3CCc4cc(Cl)ccc43)nc(-c3cccnc3)nc12] | 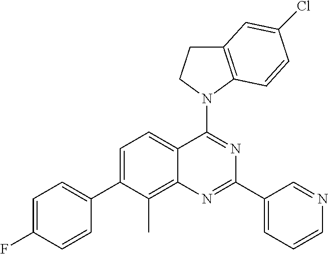 [SMILES: Cc1c(-c2ccc(F)cc2)ccc2c(N3CCc4cc(Cl)ccc43)nc(-c3cccnc3)nc12] | HCl | 1H NMR (400 MHz, DMSO) δ 9.63 (d, J = 1.5 Hz, 1H), 9.04 (d, J = 8.1 Hz, 1H), 8.86 (dd, J = 5.1, 1.5 Hz, 1H), 8.12 (d, J = 9.0 Hz, 1H), 7.86 (dd, J = 7.8, 4.9 Hz, 1H), 7.74 (d, J = 8.6 Hz, 1H), 7.62-7.27 (m, 7H), 4.63 (t, J = 8.1 Hz, 2H), 3.25 (t, J = 7.9 Hz, 2H), 2.75- 2.65 (m, 3H). |
| 119 | 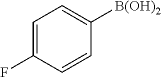 | 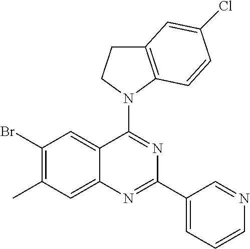 [SMILES: Cc1cc2nc(-c3cccnc3)nc(N3CCc4cc(Cl)ccc43)c2cc1Br] | 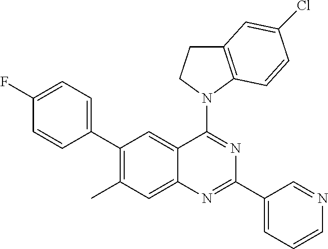 [SMILES: Cc1cc2nc(-c3cccnc3)nc(N3CCc4cc(Cl)ccc43)c2cc1-c1ccc(F)cc1] | 2HCl | 1H NMR (400 MHz, DMSO) δ 9.58 (d, J = 1.8 Hz, 1H), 9.17 (d, J = 8.4 Hz, 1H), 8.99 (d, J = 5.4 Hz, 1H), 8.11-7.99 (m, 3H), 7.90 (d, J = 8.6 Hz, 1H), 7.62-7.50 (m, 2H), 7.46 (d, J = 2.1 Hz, 1H), 7.41-7.29 (m, 3H), 4.69 (t, J = 7.9 Hz, 3H), 3.23 (t, J = 7.8 Hz, 2H), 2.46 (s, 3H). |
| 120 | 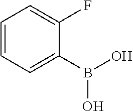 | 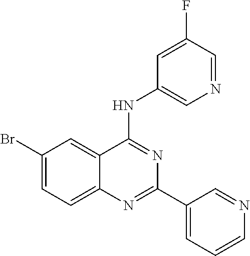 [SMILES: Fc1cncc(Nc2nc(-c3cccnc3)nc3ccc(Br)cc23)c1] | 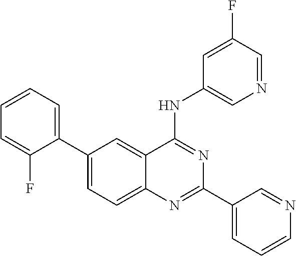 [SMILES: Fc1cncc(Nc2nc(-c3cccnc3)nc3ccc(-c4ccccc4F)cc23)c1] | HCl | 1H NMR (400 MHz, DMSO) δ 10.74 (s, 1H), 9.53 (d, J = 1.5 Hz, 1H), 9.13-9.03 (m, 1H), 8.99-8.92 (m, 1H), 8.90 (s, 1H), 8.78- 8.70 (m, 1H), 8.50 (ddd, J = 8.8, 7.3, 2.8 Hz, 1H), 8.21-8.13 (m, 1H), 8.08 (d, J = 8.7 Hz, 1H), 8.00 (dd, J = 8.0, 5.3 Hz, 1H), 7.84-7.74 (m, 1H), 7.61-7.50 (m, 1H), 7.48-7.39 (m, 2H), 7.35 (dd, J = 8.8, 3.2 Hz, 1H). |
| 121 | 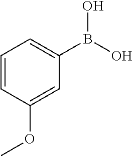 | 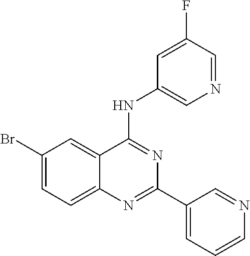 [SMILES: Fc1cncc(Nc2nc(-c3cccnc3)nc3ccc(Br)cc23)c1] | 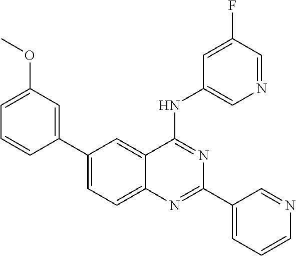 [SMILES: COc1cccc(-c2ccc3nc(-c4cccnc4)nc(Nc4cncc(F)c4)c3c2)c1] | HCl | 1H NMR (400 MHz, DMSO) δ 10.68 (s, 1H), 9.52 (d, J = 1.6 Hz, 1H), 9.02-8.94 (m, 2H), 8.88 (dd, J = 5.1, 1.4 Hz, 1H), 8.80- 8.70 (m, 1H), 8.51 (ddd, J = 8.8, 7.3, 2.8 Hz, 1H), 8.34 (dd, J = 8.7, 1.9 Hz, 1H), 8.04 (d, J = 8.7 Hz, 1H), 7.90 (dd, J = 7.9, 5.3 Hz, 1H), 7.58-7.45 (m, 3H), 7.36 (dd, J = 8.8, 3.2 Hz, 1H), 7.11-7.00 (m, 1H), 3.90 (s, 3H). |
| 122 | 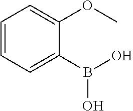 | 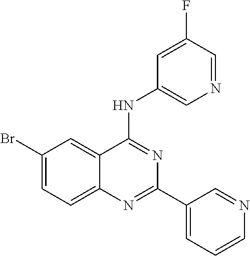 [SMILES: Fc1cncc(Nc2nc(-c3cccnc3)nc3ccc(Br)cc23)c1] | 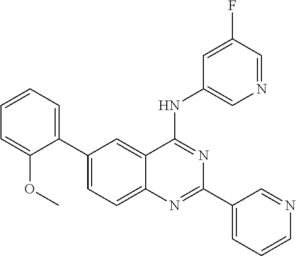 [SMILES: COc1ccccc1-c1ccc2nc(-c3cccnc3)nc(Nc3cncc(F)c3)c2c1] | HCl | 1H NMR (400 MHz, DMSO) δ 10.55 (s, 1H), 9.53 (d, J = 1.6 Hz, 1H), 9.01 (d, J = 8.1 Hz, 1H), 8.90 (dd, J = 5.2, 1.4 Hz, 1H), 8.76- 8.69 (m, 2H), 8.49 (ddd, J = 8.8, 7.3, 2.8 Hz, 1H), 8.13 (dd, J = 8.7, 1.7 Hz, 1H), 8.01 (d, J = 8.7 Hz, 1H), 7.92 (dd, J = 8.0, 5.2 Hz, 1H), 7.52 (dd, J = 7.5, 1.7 Hz, 1H), 7.49- 7.44 (m, 1H), 7.35 (dd, J = 8.8, 3.2 Hz, 1H), 7.22 (d, J = 7.7 Hz, 1H), 7.15 (td, J = 7.4, 1.0 Hz, 1H), 3.83 (s, 3H). |
| 123 | 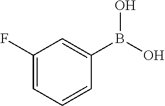 | 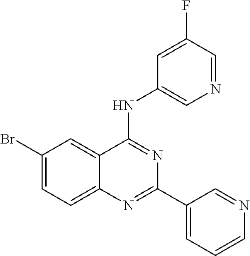 [SMILES: Fc1cncc(Nc2nc(-c3cccnc3)nc3ccc(Br)cc23)c1] | 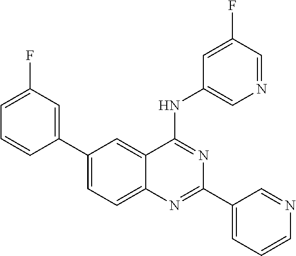 [SMILES: Fc1cncc(Nc2nc(-c3cccnc3)nc3ccc(-c4cccc(F)c4)cc23)c1] | HCl | 1H NMR (400 MHz, DMSO) δ 10.61 (s, 1H), 9.52 (d, J = 1.5 Hz, 1H), 9.00 (d, J = 1.8 Hz, 1H), 8.98-8.93 (m, 1H), 8.87 (dd, J = 5.2, 1.5 Hz, 1H), 8.77-8.73 (m, 1H), 8.51 (ddd, J = 8.8, 7.4, 2.8 Hz, 1H), 8.36 (dd, J = 8.7, 1.9 Hz, 1H), 8.04 (d, J = 8.7 Hz, 1H), 7.88 (dd, J = 8.1, 5.1 Hz, 1H), 7.86-7.79 (m, 2H), 7.68-7.59 (m, 1H), 7.37 (dd, J = 8.8, 3.2 Hz, 1H), 7.32 (td, J = 8.5, 2.1 Hz, 1H). |
| 124 | 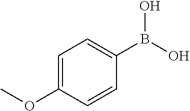 | 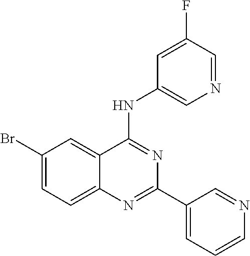 [SMILES: Fc1cncc(Nc2nc(-c3cccnc3)nc3ccc(Br)cc23)c1] | 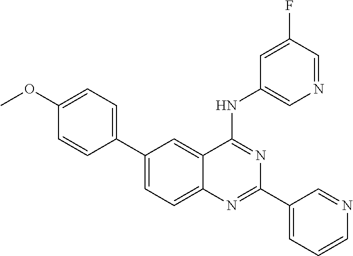 [SMILES: COc1ccc(-c2ccc3nc(-c4cccnc4)nc(Nc4cncc(F)c4)c3c2)cc1] | HCl | 1H NMR (400 MHz, DMSO) δ 10.63 (s, 1H), 9.51 (s, 1H), 8.97 (d, J = 8.2 Hz, 1H), 8.91 (d, J = 1.7 Hz, 1H), 8.89-8.85 (m, 1H), 8.77-8.73 (m, 1H), 8.50 (ddd, J = 8.8, 7.4, 2.8 Hz, 1H), 8.30 (dd, J = 8.8, 1.8 Hz, 1H), 8.02 (d, J = 8.7 Hz, 1H), 7.95-7.85 (m, 3H), 7.36 (dd, J = 8.8, 3.2 Hz, 1H), 7.18- 7.11 (m, 2H), 3.85 (s, 3H). |
| 125 | 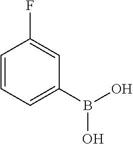 | 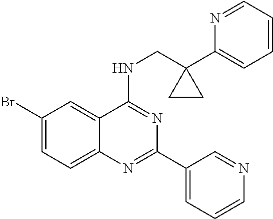 [SMILES: Brc1ccc2nc(-c3cccnc3)nc(NCC3(c4ccccn4)CC3)c2c1] | 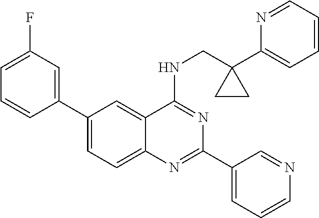 [SMILES: Fc1cccc(-c2ccc3nc(-c4cccnc4)nc(NCC4(c5ccccn5)CC4)c3c2)c1] | | 1H NMR (400 MHz, DMSO) δ 9.64 (d, J = 1.5 Hz, 1H), 8.83-8.73 (m, 1H), 8.73-8.66 (m, 2H), 8.55-8.49 (m, 1H), 8.49-8.44 (m, 1H), 8.16 (dd, J = 8.7, 1.9 Hz, 1H), 7.86 (d, J = 8.7 Hz, 1H), 7.75-7.66 (m, 2H), 7.64-7.52 (m, 3H), 7.52-7.46 (m, 1H), 7.28-7.21 (m, 1H), 7.11 (ddd, J = 7.4, 4.8, 0.9 Hz, 1H), 4.30 (d, J = 5.2 Hz, 2H), 1.24 (s, 4H). |
| 126 | 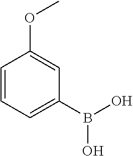 | 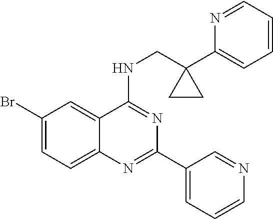 [SMILES: Brc1ccc2nc(-c3cccnc3)nc(NCC3(c4ccccn4)CC3)c2c1] | 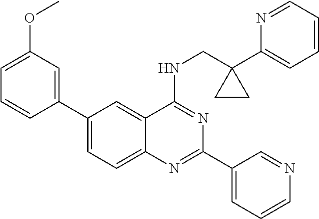 [SMILES: COc1cccc(-c2ccc3nc(-c4cccnc4)nc(NCC4(c5ccccn5)CC4)c3c2)c1] | | 1H NMR (400 MHz, DMSO) δ 9.70-9.54 (m, 1H), 8.77 (m, 1H), 8.69 (dd, J = 4.7, 1.7 Hz, 1H), 8.65 (d, J = 1.8 Hz, 1H), 8.55-8.49 (m, 1H), 8.49-8.44 (m, 1H), 8.13 (dd, J = 8.7, 1.9 Hz, 1H), 7.86 (d, J = 8.7 Hz, 1H), 7.64-7.58 (m, 1H), 7.56 (dd, J = 7.9, 4.8 Hz, 1H), 7.52-7.48 (m, 1H), 7.48-7.33 (m, 3H), 7.11 (ddd, J = 7.4, 4.8, 1.0 Hz, 1H), 7.04-6.96 (m, 1H), 4.29 (d, J = 5.3 Hz, 2H), 3.86 (s, 3H), 1.23 (s, 4H). |
| 127 | 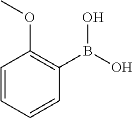 | 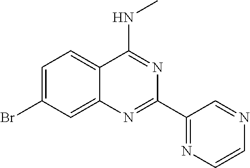 [SMILES: CNc1nc(-c2cnccn2)nc2cc(Br)ccc12] | 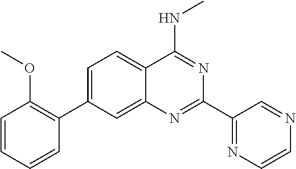 [SMILES: CNc1nc(-c2cnccn2)nc2cc(-c3ccccc3OC)ccc12] | HCl | 1H NMR (400 MHz, DMSO) δ 10.66 (s, 1H), 9.85 (d, J = 1.3 Hz, 1H), 9.07 (d, J = 2.4 Hz, 1H), 9.01 (dd, J = 2.4, 1.5 Hz, 1H), 8.64 (d, J = 8.7 Hz, 1H), 8.44 (d, J = 1.3 Hz, 1H), 7.93 (dd, J = 8.7, 1.5 Hz, 1H), 7.55-7.43 (m, 2H), 7.24 (d, J = 8.1 Hz, 1H), 7.20- 7.05 (m, 1H), 3.86 (s, 3H), 3.38 (d, J = 4.7 Hz, 3H). |
| 128 | 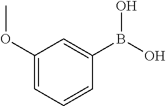 | 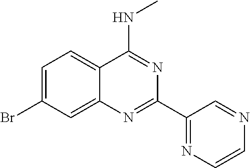 [SMILES: CNc1nc(-c2cnccn2)nc2cc(Br)ccc12] | 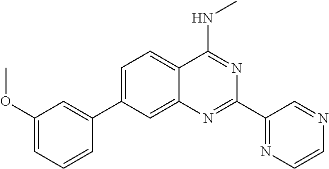 [SMILES: CNc1nc(-c2cnccn2)nc2cc(-c3cccc(OC)c3)ccc12] | | 1H NMR (400 MHz, DMSO) δ 9.66 (d, J = 1.4 Hz, 1H), 8.82 (dd, J = 2.4, 1.5 Hz, 1H), 8.76 (d, J = 2.4 Hz, 1H), 8.58 (d, J = 4.6 Hz, 1H), 8.34 (d, J = 8.6 Hz, 1H), 8.11 (d, J = 1.8 Hz, 1H), 7.92 (dd, J = 8.6, 1.9 Hz, 1H), 7.50- 7.36 (m, 3H), 7.08-6.95 (m, 1H), 3.88 (s, 3H), 3.17 (d, J = 4.5 Hz, 3H). |
| 129 | 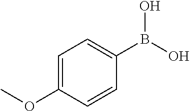 | 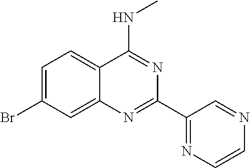 [SMILES: CNc1nc(-c2cnccn2)nc2cc(Br)ccc12] | 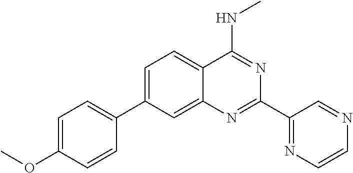 [SMILES: CNc1nc(-c2cnccn2)nc2cc(-c3ccc(OC)cc3)ccc12] | HCl | 1H NMR (400 MHz, DMSO) δ 10.44 (brs, 1H), 9.84 (d, J = 1.1 Hz, 1H), 9.06 (d, J = 2.1 Hz, 1H), 9.03-8.95 (m, 1H), 8.58 (d, J = 8.7 Hz, 1H), 8.47 (s, 1H), 8.13 (d, J = 7.2 Hz, 1H), 7.82 (d, J = 8.6 Hz, 2H), 7.16 (d, J = 8.6 Hz, 2H), 3.86 (s, 3H), 3.39-3.36 (m, 3H). |
| 130 | 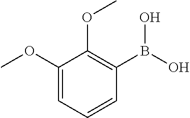 | 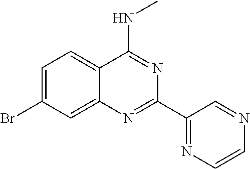 [SMILES: CNc1nc(-c2cnccn2)nc2cc(Br)ccc12] | 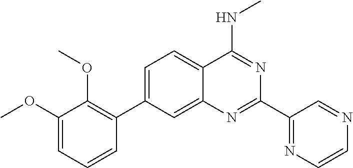 [SMILES: CNc1nc(-c2cnccn2)nc2cc(-c3cccc(OC)c3OC)ccc12] | HCl | 1H NMR (400 MHz, DMSO) δ 10.81 (s, 1H), 9.85 (d, J = 1.3 Hz, 1H), 9.07 (d, J = 2.4 Hz, 1H), 9.01 (dd, J = 2.3, 1.5 Hz, 1H), 8.72 (d, J = 8.6 Hz, 1H), 8.37 (d, J = 1.4 Hz, 1H), 7.88 (dd, J = 8.6, 1.6 Hz, 1H), 7.29-7.16 (m, 2H), 7.05 (dd, J = 7.2, 2.0 Hz, 1H), 3.90 (s, 3H), 3.69 (s, 3H), 3.38 (d, J = 4.6 Hz, 3H). |
| 131 | 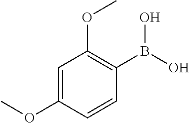 | 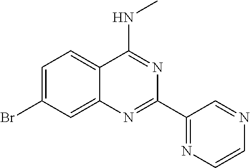 [SMILES: CNc1nc(-c2cnccn2)nc2cc(Br)ccc12] | 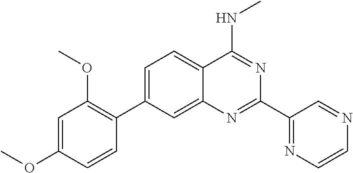 [SMILES: CNc1nc(-c2cnccn2)nc2cc(-c3ccc(OC)cc3OC)ccc12] | HCl | 1H NMR (400 MHz, DMSO) δ 11.04-10.76 (m, 1H), 9.81 (d, J = 1.3 Hz, 1H), 9.07 (d, J = 2.4 Hz, 1H), 9.00 (dd, J = 2.2, 1.5 Hz, 1H), 8.70 (d, J = 8.7 Hz, 1H), 8.42 (d, J = 1.4 Hz, 1H), 7.88 (dd, J = 8.6, 1.5 Hz, 1H), 7.42 (d, J = 8.4 Hz, 1H), 6.76 (d, J = 2.3 Hz, 1H), 6.71 (dd, J = 8.5, 2.3 Hz, 1H), 3.87 (s, 3H), 3.86 (s, 3H), 3.36 (d, J = 4.6 Hz, 3H). |
| 132 | 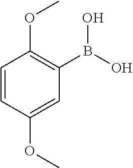 | 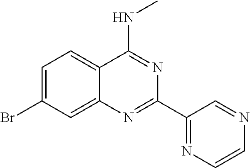 [SMILES: CNc1nc(-c2cnccn2)nc2cc(Br)ccc12] | 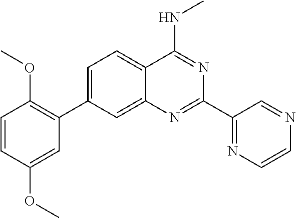 [SMILES: CNc1nc(-c2cnccn2)nc2cc(-c3cc(OC)ccc3OC)ccc12] | HCl | 1H NMR (400 MHz, DMSO) δ 10.62 (s, 1H), 9.86 (d, J = 1.3 Hz, 1H), 9.08 (d, J = 2.4 Hz, 1H), 9.04-8.99 (m, 1H), 8.61 (d, J = 8.7 Hz, 1H), 8.46 (d, J = 1.1 Hz, 1H), 7.96 (dd, J = 8.6, 1.5 Hz, 1H), 7.18 (d, J = 8.8 Hz, 1H), 7.11-7.01 (m, 2H), 3.80 (s, 3H), 3.80 (s, 3H), 3.39 (d, J = 2.0 Hz, 3H). |
| 133 | 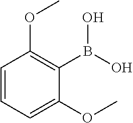 | 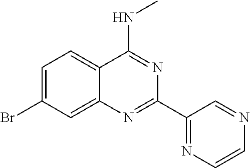 [SMILES: CNc1nc(-c2cnccn2)nc2cc(Br)ccc12] | 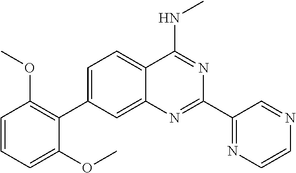 [SMILES: CNc1nc(-c2cnccn2)nc2cc(-c3c(OC)cccc3OC)ccc12] | HCl | 1H NMR (400 MHz, DMSO) δ 10.84 (s, 1H), 9.84 (d, J = 1.4 Hz, 1H), 9.07 (d, J = 2.4 Hz, 1H), 9.00 (dd, J = 2.4, 1.5 Hz, 1H), 8.68 (d, J = 8.6 Hz, 1H), 8.17 (d, J = 1.4 Hz, 1H), 7.67 (dd, J = 8.5, 1.5 Hz, 1H), 7.47-7.38 (m, 1H), 6.85 (d, J = 8.5 Hz, 2H), 3.73 (s, 6H), 3.37 (d, J = 4.6 Hz, 3H). |
| 134 | 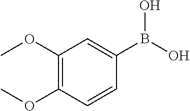 | 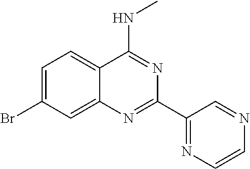 [SMILES: CNc1nc(-c2cnccn2)nc2cc(Br)ccc12] | 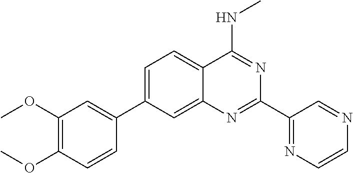 [SMILES: CNc1nc(-c2cnccn2)nc2cc(-c3ccc(OC)c(OC)c3)ccc12] | HCl | 1H NMR (400 MHz, DMSO) δ 10.43 (s, 1H), 9.85 (d, J = 1.1 Hz, 1H), 9.06 (d, J = 2.2 Hz, 1H), 9.04-8.97 (m, 1H), 8.58 (d, J = 8.7 Hz, 1H), 8.50 (s, 1H), 8.18 (d, J = 7.3 Hz, 1H), 7.49-7.37 (m, 2H), 7.17 (d, J = 8.3 Hz, 1H), 3.91 (s, 3H), 3.85 (s, 3H), 3.37 (d, J = 4.7 Hz, 3H). |
| 135 | 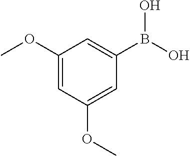 | 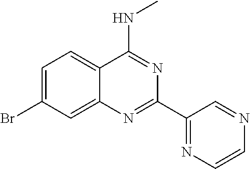 [SMILES: CNc1nc(-c2cnccn2)nc2cc(Br)ccc12] | 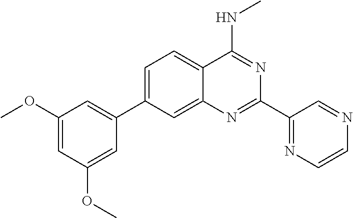 [SMILES: CNc1nc(-c2cnccn2)nc2cc(-c3cc(OC)cc(OC)c3)ccc12] | HCl | 1H NMR (400 MHz, DMSO) δ 10.75 (s, 1H), 9.83 (s, 1H), 9.07 (d, J = 2.1 Hz, 1H), 9.01 (s, 1H), 8.69 (d, J = 8.6 Hz, 1H), 8.50 (s, 1H), 8.15 (d, J = 8.2 Hz, 1H), 6.94 (d, J = 1.7 Hz, 2H), 6.64 (s, 1H), 3.85 (s, 6H), 3.37 (d, J = 4.4 Hz, 3H). |

| | ¹H NMR | Purity | Method | | Retention | LCMS |
|---|---|---|---|---|---|---|
| Number | Solvent | Percent | of Coupling | LCMS | Time | Method |
| 72 | DMSO | >98 | Method L | | | |
| 73 | DMSO | >98 | Method L | | | |
| 74 | DMSO | >98 | Method L | | | |
| 75 | DMSO | >98 | Method L | | | |
| 76 | DMSO | >98 | Method L | | | |
| 77 | DMSO | >98 | Method L | | | |
| 78 | DMSO | >98 | Method L | | | |
| 79 | DMSO | >98 | Method L | | | |
| 80 | DMSO | >98 | Method L | | | |
| 81 | DMSO | >98 | Method L | | | |
| 82 | DMSO | >98 | Method L | | | |
| 83 | DMSO | >98 | Method L | | | |
| 84 | DMSO | >98 | Method L | | | |
| 85 | DMSO | >98 | Method L | | | |
| 86 | DMSO | >98 | Method L | | | |
| 87 | DMSO | >98 | Method L | | | |
| 88 | DMSO | >98 | Method L | | | |
| 89 | DMSO | >98 | Method L | | | |
| 90 | DMSO | >98 | Method L | | | |
| 91 | DMSO | >98 | L | | | |
| 92 | DMSO | >98 | L | | | |
| 93 | DMSO | >98 | L | | | |
| 94 | DMSO | >98 | L | | | |
| 95 | DMSO | >98 | L | | | |
| 96 | DMSO | >98 | L | | | |
| 97 | DMSO | >98 | L | | | |
| 98 | DMSO | >98 | L | | | |
| 99 | DMSO | >98 | L | | | |
| 100 | DMSO | >98 | L | | | |
| 101 | DMSO | >98 | L | | | |
| 102 | DMSO | >98 | L | | | |
| 103 | CDCl3 | >98 | L | | | |
| 104 | CDCl3 | >98 | L | | | |
| 105 | DMSO | >98 | L | | | |
| 106 | DMSO | >98 | L | | | |
| 107 | DMSO | >98 | L | | | |
| 108 | DMSO | >98 | L | | | |
| 109 | DMSO | >98 | L | | | |
| 110 | DMSO | >98 | L | | | |
| 111 | DMSO | >98 | L | | | |
| 112 | DMSO | >98 | L | | | |
| 113 | DMSO | >98 | L | | | |
| 114 | DMSO | >98 | L | | | |
| 115 | DMSO | >98 | L | | | |
| 116 | DMSO | >98 | L | | | |
| 117 | DMSO | >98 | L | | | |
| 118 | DMSO | >98 | L | | | |
| 119 | DMSO | >98 | L | | | |
| 120 | DMSO | >98 | L | | | |
| 121 | DMSO | >98 | L | | | |
| 122 | DMSO | >98 | L | | | |
| 123 | DMSO | >98 | L | | | |
| 124 | DMSO | >98 | L | | | |
| 125 | DMSO | >98 | L | | | |
| | | | Temperature | | | |
| | | | at 100° C. | | | |
| 126 | DMSO | >98 | L | | | |
| | | | Temperature | | | |
| | | | at 100° C. | | | |
| 127 | DMSO | >98 | L | | | |
| | | | Temperature | | | |
| | | | at 100° C. | | | |
| 128 | DMSO | >98 | L | | | |
| | | | Temperature | | | |
| | | | at 100° C. | | | |
| 129 | DMSO | >98 | L | | | |
| | | | Temperature | | | |
| | | | at 100° C. | | | |
| 130 | DMSO | >98 | L | | | |
| | | | Temperature | | | |
| | | | at 100° C. | | | |
| 131 | DMSO | >98 | L | | | |
| | | | Temperature | | | |
| | | | at 100° C. | | | |
| 132 | DMSO | >98 | L | | | |
| | | | Temperature | | | |
| | | | at 100° C. | | | |
| 133 | DMSO | >98 | L | | | |
| | | | Temperature | | | |
| | | | at 100° C. | | | |
| 134 | DMSO | >98 | L | | | |
| | | | Temperature | | | |
| | | | at 100° C. | | | |
| 135 | DMSO | >98 | L | | | |
| | | | Temperature | | | |
| | | | at 100° C. | | | |

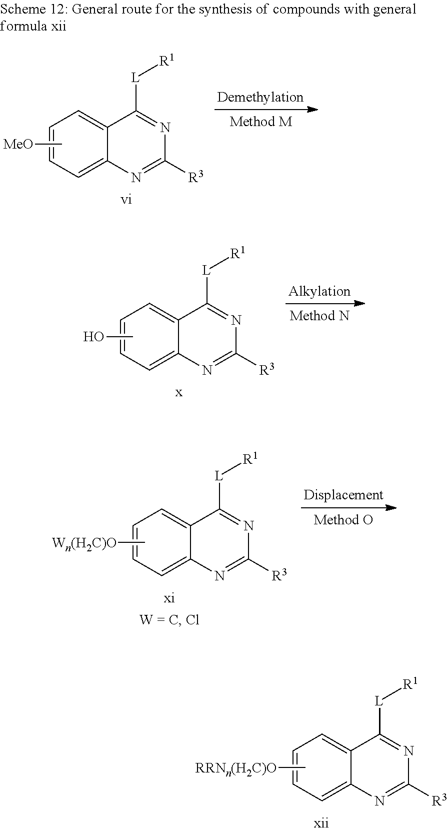

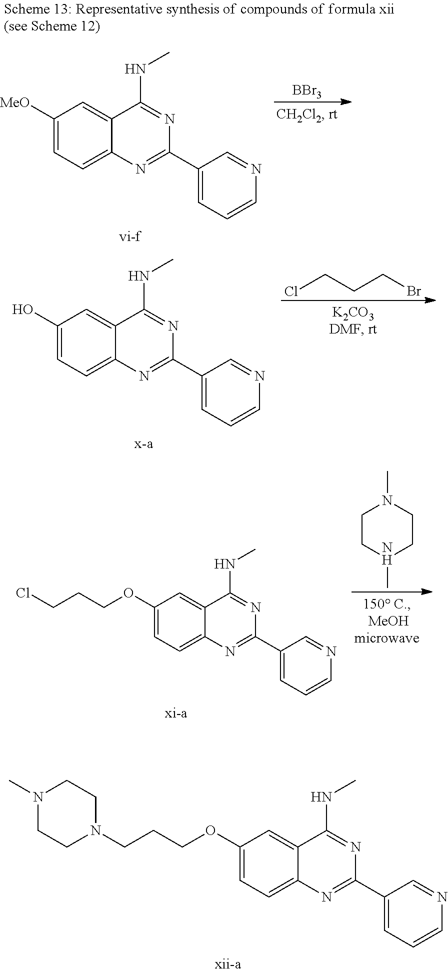

### Method M: 4-(methylamino)-2-(pyridin-3-yl)quinazolin-6-ol (x-a)

To 6-methoxy-N-methyl-2-(pyridin-3-yl)quinazolin-4-amine (1.600 g, 6.01 mmol) was added 1M solution of boron tribromide in dichloromethane (30.0 mL, 30.0 mmol), slowly. The mixture was stirred for 4 days at room temperature. The reaction mixture was poured into an ice-cooled solution of aqueous NaHCO₃ and stirred. A precipitate formed which was collected by filtration and dried to give 1.5 g of the desired product as a yellow solid in a 99% yield. LCMS m/z=253 (M+1) (Method D) (retention time=2.04 min) ¹H NMR (300 MHz, DMSO) δ 10.18 (s, 1H), 9.54 (d, J=1.4 Hz, 1H), 8.78-8.67 (m, 2H), 8.60 (s, 1H), 7.72 (d, J=8.9 Hz, 1H), 7.58 (dd, J=7.6, 5.1 Hz, 1H), 7.50 (d, J=2.4 Hz, 1H), 7.39 (dd, J=9.0, 2.5 Hz, 1H), 3.15 (d, J=4.4 Hz, 3H).

### Method N: 6-(3-chloropropoxy)-N-methyl-2-(pyridin-3-yl)quinazolin-4-amine (xi-a)

To a suspension of 4-(methylamino)-2-(pyridin-3-yl)quinazolin-6-ol (0.200 g, 0.793 mmol) and potassium carbonate (1.096 g, 7.93 mmol) in DMF (5 ml) was added 1-bromo-3-chloropropane (0.781 ml, 7.93 mmol). The mixture was stirred overnight at room temperature. The reaction was diluted with water (10 mL) and extracted with ethyl acetate (2×10 mL). The combined organics were washed with water (1×20 mL) and brine (1×15 mL) and then dried over MgSO₄, filtered and concentrated. The residue was triturated in a CH₂Cl₂/hexane mixture, followed by evaporation of only the CH₂Cl₂ to form a suspended solid. The precipitate was collected by filtration and dried to give 0.166 g of the desired product as a pale yellow solid in a 64% yield. LCMS m/z=329 (M+1) (Method C) (retention time=2.03 min) ¹H NMR (300 MHz, DMSO) δ 9.59 (s, 1H), 8.72 (d, J=8.0 Hz, 1H), 8.64 (d, J=3.9 Hz, 1H), 8.29 (d, J=4.1 Hz, 1H), 7.79-7.61 (m, 2H), 7.50 (dd, J=7.7, 5.0 Hz, 1H), 7.42 (dd, J=9.0, 2.2 Hz, 1H), 4.21 (t, J=5.9 Hz, 2H), 3.85 (t, J=6.3 Hz, 2H), 3.14 (d, J=4.2 Hz, 3H), 2.31-2.16 (m, 2H).

### Method O: N-methyl-6-(3-(4-methylpiperazin-1-yl)propoxy)-2-(pyridin-3-yl)quinazolin-4-amine tetrahydrochloride (xii-a)

To a 10 mL microwave vial was added 6-(3-chloropropoxy)-N-methyl-2-(pyridin-3-yl)quinazolin-4-amine (0.160 g, 0.487 mmol) and 1-methyl piperazine (0.540 ml, 4.87 mmol) in methanol (3 ml) to give a brown solution. The mixture was heated under μW condition at 150° C. for 20 min. The reaction mixture was diluted with water (10 mL) and extracted with ethyl acetate (2×10 mL). The combined organic layers were washed with brine (1×15 mL) and dried over MgSO₄, filtered and concentrated. The residue was purified via ISCO (amine silica gel, 2:1 to 0:1 Hex/EtOAc; 14 gm Gold column). The product was converted to the HCl salt by treatment with 4 M HCl-dioxane. The HCl salt was washed with methanol to give 76 mg of the desired product as a light yellow solid in a 29% yield. LCMS m/z=393 (M+1) (Method C) (retention time=1.30 min) ¹H NMR (300 MHz, CDCl₃) δ 9.81-9.70 (m, 1H), 8.79 (dt, J=8.0, 1.9 Hz, 1H), 8.67 (dd, J=4.8, 1.7 Hz, 1H), 7.84 (d, J=9.1 Hz, 1H), 7.45-7.33 (m, 2H), 6.99 (d, J=2.5 Hz, 1H), 5.83 (s, 1H), 4.08 (t, J=6.2 Hz, 2H), 3.30 (d, J=4.8 Hz, 3H), 2.76-2.33 (m, 10H), 2.29 (s, 3H), 2.09-1.95 (m, 2H).

The compounds in the following table were prepared in a manner analogous to that described in Scheme 13 substituting 1-bromo-3-chloropropane with appropriate nucleophile.

TABLE 3

| | Starting | Starting | |
|---|---|---|---|
| Number | Material 1 | Material 2 | Product |
| 136 | 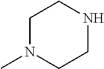 | 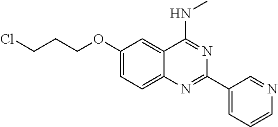 [SMILES: CNc1nc(-c2cccnc2)nc2ccc(OCCCCl)cc12] | 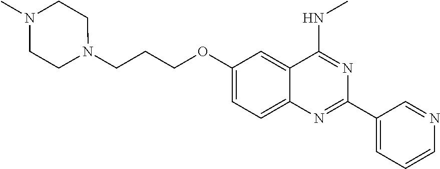 [SMILES: CNc1nc(-c2cccnc2)nc2ccc(OCCCN3CCN(C)CC3)cc12] |
| 137 | 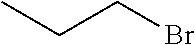 | 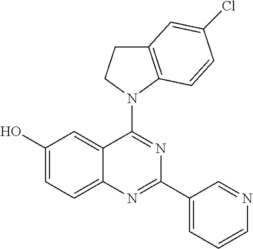 [SMILES: Oc1ccc2nc(-c3cccnc3)nc(N3CCc4cc(Cl)ccc43)c2c1] | 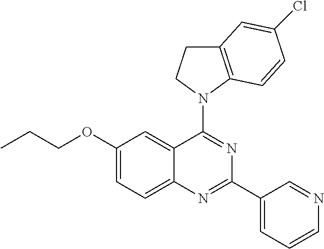 [SMILES: CCCOc1ccc2nc(-c3cccnc3)nc(N3CCc4cc(Cl)ccc43)c2c1] |
| 138 | 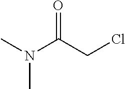 | 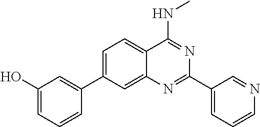 [SMILES: CNc1nc(-c2cccnc2)nc2cc(-c3cccc(O)c3)ccc12] | 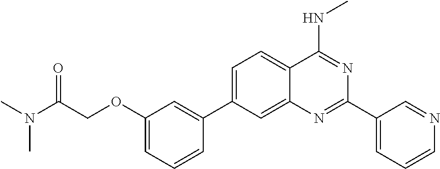 [SMILES: CNc1nc(-c2cccnc2)nc2cc(-c3cccc(OCC(=O)N(C)C)c3)ccc12] |
| 139 | Ethyl iodide | 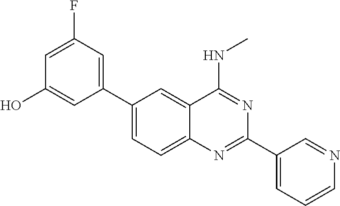 [SMILES: CNc1nc(-c2cccnc2)nc2ccc(-c3cc(O)cc(F)c3)cc12] | 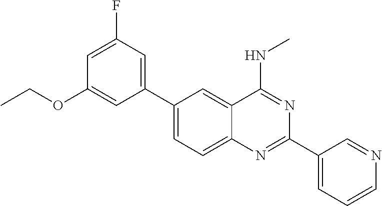 [SMILES: CCOc1cc(F)cc(-c2ccc3nc(-c4cccnc4)nc(NC)c3c2)c1] |
| 140 | 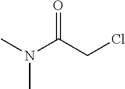 | 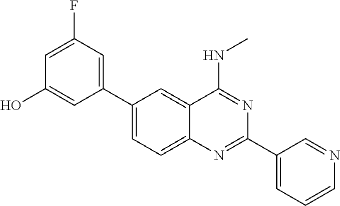 [SMILES: CNc1nc(-c2cccnc2)nc2ccc(-c3cc(O)cc(F)c3)cc12] | 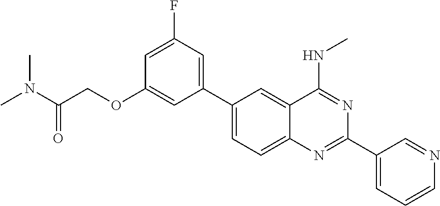 [SMILES: CNc1nc(-c2cccnc2)nc2ccc(-c3cc(F)cc(OCC(=O)N(C)C)c3)cc12] |
| 141 | Ethyl iodide | 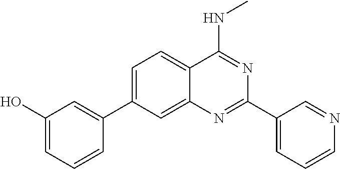 [SMILES: CNc1nc(-c2cccnc2)nc2cc(-c3cccc(O)c3)ccc12] | 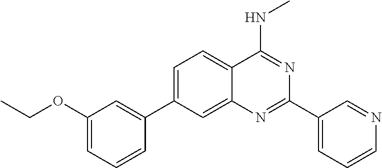 [SMILES: CCOc1cccc(-c2ccc3c(NC)nc(-c4cccnc4)nc3c2)c1] |
| 142 | 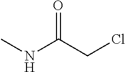 | 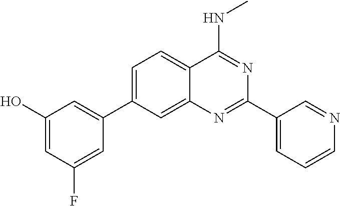 [SMILES: CNc1nc(-c2cccnc2)nc2cc(-c3cc(O)cc(F)c3)ccc12] | 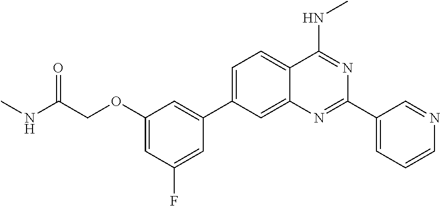 [SMILES: CNC(=O)COc1cc(F)cc(-c2ccc3c(NC)nc(-c4cccnc4)nc3c2)c1] |
| 143 | 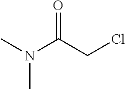 | 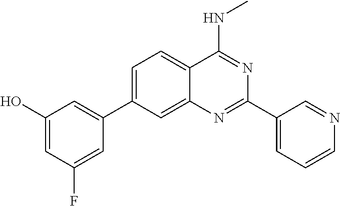 [SMILES: CNc1nc(-c2cccnc2)nc2cc(-c3cc(O)cc(F)c3)ccc12] | 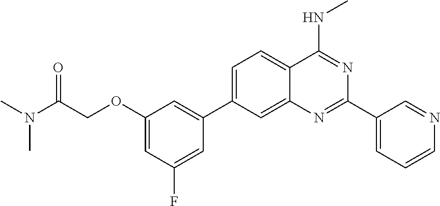 [SMILES: CNc1nc(-c2cccnc2)nc2cc(-c3cc(F)cc(OCC(=O)N(C)C)c3)ccc12] |
| 144 | 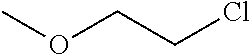 | 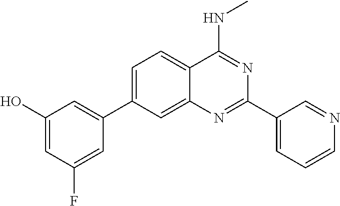 [SMILES: CNc1nc(-c2cccnc2)nc2cc(-c3cc(O)cc(F)c3)ccc12] | 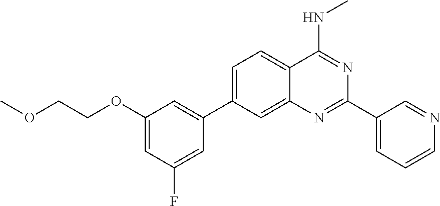 [SMILES: CNc1nc(-c2cccnc2)nc2cc(-c3cc(F)cc(OCCOC)c3)ccc12] |
| 145 | Ethyl iodide | 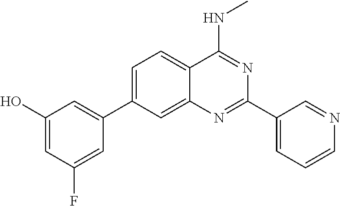 [SMILES: CNc1nc(-c2cccnc2)nc2cc(-c3cc(O)cc(F)c3)ccc12] | 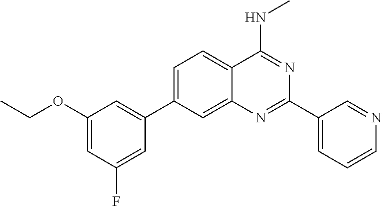 [SMILES: CCOc1cc(F)cc(-c2ccc3c(NC)nc(-c4cccnc4)nc3c2)c1] |
| 146 | 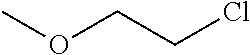 | 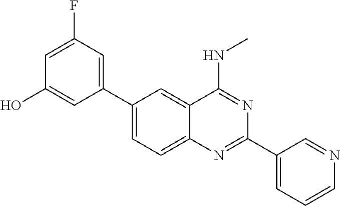 [SMILES: CNc1nc(-c2cccnc2)nc2ccc(-c3cc(O)cc(F)c3)cc12] | 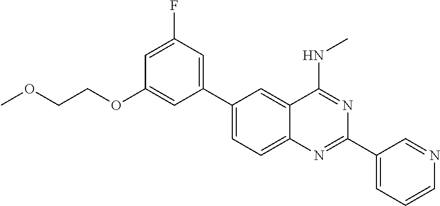 [SMILES: CNc1nc(-c2cccnc2)nc2ccc(-c3cc(F)cc(OCCOC)c3)cc12] |
| 147 | 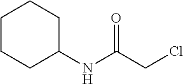 [SMILES: O=C(CCl)NC1CCCCC1] | 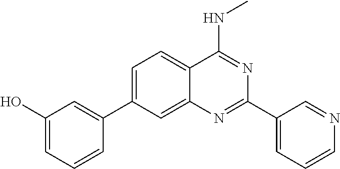 [SMILES: CNc1nc(-c2cccnc2)nc2cc(-c3cccc(O)c3)ccc12] | 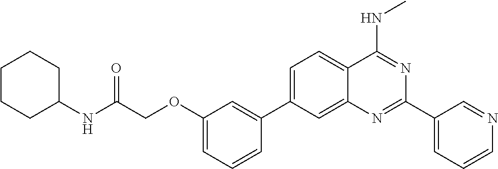 [SMILES: CNc1nc(-c2cccnc2)nc2cc(-c3cccc(OCC(=O)NC4CCCCC4)c3)ccc12] |
| 148 | 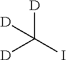 | 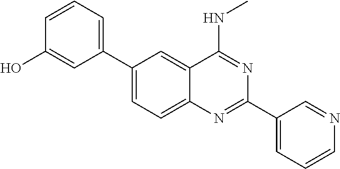 [SMILES: CNc1nc(-c2cccnc2)nc2ccc(-c3cccc(O)c3)cc12] | 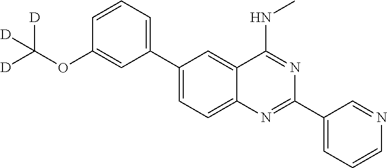 [SMILES: [2H]C([2H])([2H])Oc1cccc(-c2ccc3nc(-c4cccnc4)nc(NC)c3c2)c1] |
| 149 | 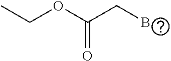 | 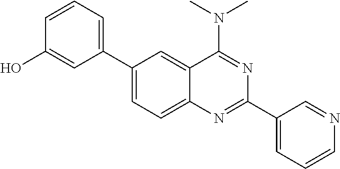 [SMILES: CN(C)c1nc(-c2cccnc2)nc2ccc(-c3cccc(O)c3)cc12] | 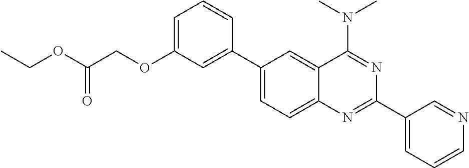 [SMILES: CCOC(=O)COc1cccc(-c2ccc3nc(-c4cccnc4)nc(N(C)C)c3c2)c1] |

| | | | |
|---|---|---|---|
| 150 | 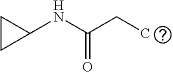 | 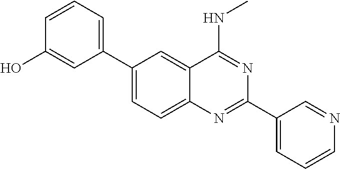 [SMILES: CNc1nc(-c2cccnc2)nc2ccc(-c3cccc(O)c3)cc12] | 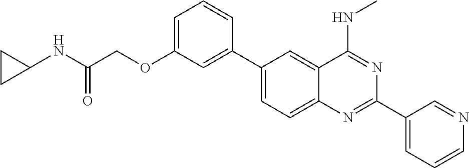 [SMILES: CNc1nc(-c2cccnc2)nc2ccc(-c3cccc(OCC(=O)NC4CC4)c3)cc12] |

| | Salt | | ¹H NMR | Purity | Method | | Retention | LCMS |
|---|---|---|---|---|---|---|---|---|
| Number | type | ¹H NMR | Solvent | percent | of Coupling | LCMS | Time | Method |
| 136 | 4HCl | 1H NMR (300 MHz, D2O) δ 9.52 (s, | D₂O | 99 | Method | 393 | 1.30 | Method C |
| | | 1H), 9.08 (d, J = 8.3 Hz, 1H), 8.96 | | | N, O | (M + 1) | | |
| | | (d, J = 5.0 Hz, 1H), 8.08 (t, J = 6.6 | | | | | | |
| | | Hz, 1H), 7.86 (d, J = 9.0 Hz, 1H), | | | | | | |
| | | 7.64 (d, J = 9.6 Hz, 1H), 7.58 (s, 1H), | | | | | | |
| | | 4.37-4.22 (m, 2H), 4.06-3.45 (m, | | | | | | |
| | | 10H), 3.39-3.31 (m, 3H), 3.05 (s, | | | | | | |
| | | 3H), 2.45-2.27 (m, 2H). | | | | | | |
| 137 | 2 HCl | 1H NMR (400 MHz, DMSO) δ 9.56 (d, | DMSO | >98 | Method N | | | |
| | | J = 1.8 Hz, 1H), 9.23 (d, J = 8.2 Hz, | | | Using | | | |
| | | 1H), 8.99 (dd, J = 5.5, 1.2 Hz, 1H), | | | Cs₂CO₃ | | | |
| | | 8.15-8.00 (m, 2H), 7.76-7.63 (m, | | | instead of | | | |
| | | 2H), 7.48 (dd, J = 6.8, 2.4 Hz, 2H), | | | K₂CO₃ | | | |
| | | 7.32 (dd, J = 8.6, 2.3 Hz, 1H), 4.67 | | | | | | |
| | | (t, J = 8.0 Hz, 3H), 4.09 (t, J = 6.5 | | | | | | |
| | | Hz, 2H), 3.26 (t, J = 7.8 Hz, 2H), | | | | | | |
| | | 1.87-1.73 (m, 2H), 1.02 (t, J = 7.5 | | | | | | |
| | | Hz, 3H). | | | | | | |
| 138 | 2HCl | 1H NMR (300 MHz, DMSO) δ 10.32- | DMSO | 99 | Method N | 414 | 1.32 | Method D |
| | | 9.98 (m, 1H), 9.64 (d, J = 2.0 Hz, | | | | (M + 1) | | |
| | | 1H), 9.02 (d, J = 5.9 Hz, 1H), 8.94 | | | | | | |
| | | (d, J = 3.6 Hz, 1H), 8.56 (d, J = 8.7 | | | | | | |
| | | Hz, 1H), 8.42 (s, 1H), 8.08 (d, J = 7.6 | | | | | | |
| | | Hz, 1H), 7.96-7.77 (m, 1H), 7.60- | | | | | | |
| | | 7.31 (m, 3H), 7.04 (d, J = 7.8 Hz, | | | | | | |
| | | 1H), 4.94 (s, 2H), 3.29 (d, J = 4.0 Hz, | | | | | | |
| | | 3H), 3.03 (s, 3H), 2.85 (s, 3H). | | | | | | |
| 139 | 2HCl | 1H NMR (300 MHz, DMSO) δ 9.94 (s, | DMSO | 97 | Method N | 375 | 1.71 | Method D |
| | | 1H), 9.61 (d, J = 2.0 Hz, 1H), 8.96 | | | | (M + 1) | | |
| | | (d, J = 8.2 Hz, 1H), 8.90 (dd, J = 5.0, | | | | | | |
| | | 1.4 Hz, 1H), 8.82 (s, 1H), 8.36 (dd, J = | | | | | | |
| | | 8.8, 1.5 Hz, 1H), 8.08 (d, J = 8.6 | | | | | | |
| | | Hz, 1H), 7.82 (dd, J = 7.8, 4.6 Hz, | | | | | | |
| | | 1H), 7.45-7.23 (m, 2H), 6.92 (d, J = | | | | | | |
| | | 11.0 Hz, 1H), 4.16 (q, J = 7.0 Hz, | | | | | | |
| | | 2H), 3.28 (d, J = 4.4 Hz, 3H), 1.37 (t, | | | | | | |
| | | J = 6.9 Hz, 3H). | | | | | | |
| 140 | 2HCl | 1H NMR (300 MHz, DMSO) δ 10.25 | DMSO | 99 | Method N | 432 | 1.43 | Method D |
| | | (s, 1H), 9.61 (s, 1H), 9.11-8.81 (m, | | | | (M + 1) | | |
| | | 3H), 8.36 (d, J = 8.3 Hz, 1H), 8.14 | | | | | | |
| | | (d, J = 8.5 Hz, 1H), 7.94-7.74 (m, | | | | | | |
| | | 1H), 7.51-7.24 (m, 2H), 6.91 (d, J = | | | | | | |
| | | 10.6 Hz, 1H), 5.01 (s, 2H), 3.28 (d, J = | | | | | | |
| | | 3.7 Hz, 3H), 3.03 (s, 3H), 2.85 (s, | | | | | | |
| | | 3H). | | | | | | |
| 141 | 2HCl | 1H NMR (300 MHz, DMSO) δ 9.92 (s, | DMSO | 99 | Method N | 357 | 1.57 | Method D |
| | | 1H), 9.62 (d, J = 2.2 Hz, 1H), 9.06- | | | | (M + 1) | | |
| | | 8.82 (m, 2H), 8.50 (d, J = 8.7 Hz, | | | | | | |
| | | 1H), 8.34 (s, 1H), 8.08 (d, J = 8.1 Hz, | | | | | | |
| | | 1H), 7.81 (dd, J = 7.6, 4.8 Hz, 1H), | | | | | | |
| | | 7.48 (t, J = 7.8 Hz, 1H), 7.43-7.29 | | | | | | |
| | | (m, 2H), 7.06 (dd, J = 8.0, 2.4 Hz, | | | | | | |
| | | 1H), 4.14 (q, J = 6.9 Hz, 2H), 3.28 | | | | | | |
| | | (d, J = 4.1 Hz, 3H), 1.37 (t, J = 6.9 | | | | | | |
| | | Hz, 3H). | | | | | | |
| 142 | 2HCl | 1H NMR (300 MHz, DMSO) δ 10.23 | DMSO | 99 | Method N | 418 | 1.41 | Method D |
| | | (s, 1H), 9.68 (s, 1H), 9.14 (d, J = 7.4 | | | | (M + 1) | | |
| | | Hz, 1H), 8.97 (d, J = 5.0 Hz, 1H), | | | | | | |
| | | 8.61 (d, J = 8.7 Hz, 1H), 8.50 (s, 1H), | | | | | | |
| | | 8.17 (d, J = 4.8 Hz, 1H), 8.08 (d, J = | | | | | | |
| | | 8.6 Hz, 1H), 7.92 (dd, J = 7.4, 5.2 | | | | | | |
| | | Hz, 1H), 7.40-7.19 (m, 2H), 6.99 (d, | | | | | | |
| | | J = 10.7 Hz, 1H), 4.63 (s, 2H), 3.28 | | | | | | |
| | | (d, J = 3.8 Hz, 3H), 2.67 (d, J = 4.4 | | | | | | |
| | | Hz, 3H). | | | | | | |
| 143 | 2HCl | 1H NMR (300 MHz, DMSO) δ 9.89 (s, | DMSO | 99 | Method N | 432 | 1.44 | Method D |
| | | 1H), 9.70-9.59 (m, 1H), 9.02 (d, J = | | | | (M + 1) | | |
| | | 7.4 Hz, 1H), 8.92 (d, J = 5.0 Hz, 1H), | | | | | | |
| | | 8.52 (d, J = 7.9 Hz, 1H), 8.36 (s, 1H), | | | | | | |
| | | 8.07 (d, J = 7.9 Hz, 1H), 7.95-7.76 | | | | | | |
| | | (m, 1H), 7.35-7.16 (m, 2H), 6.96 (d, | | | | | | |
| | | J = 10.8 Hz, 1H), 4.98 (s, 2H), 3.27 | | | | | | |
| | | (d, J = 4.2 Hz, 3H), 3.01 (s, 3H), 2.85 | | | | | | |
| | | (s, 3H). | | | | | | |
| 144 | 2HCl | 1H NMR (300 MHz, DMSO) δ 9.92 (s, | DMSO | 99 | Method N | 405 | 1.59 | Method D |
| | | 1H), 9.70-9.59 (m, 1H), 9.01 (d, J = | | | DABCO as | (M + 1) | | |
| | | 8.2 Hz, 1H), 8.91 (d, J = 5.0 Hz, 1H), | | | additive at | | | |
| | | 8.50 (d, J = 8.1 Hz, 1H), 8.40 (s, 1H), | | | 80° C. | | | |
| | | 8.07 (d, J = 8.5 Hz, 1H), 7.92-7.74 | | | | | | |
| | | (m, 1H), 7.32-7.16 (m, 2H), 6.98 (d, | | | | | | |
| | | J = 10.9 Hz, 1H), 4.32-4.14 (m, | | | | | | |
| | | 2H), 3.80-3.59 (m, 2H), 3.44-3.15 | | | | | | |
| | | (m, 6H). | | | | | | |
| 145 | 2HCl | 1H NMR (300 MHz, DMSO) δ 9.97- | DMSO | 99 | Method N | 375 | 1.71 | Method D |
| | | 9.52 (m, 2H), 8.99 (d, J = 7.8 Hz, | | | | (M + 1) | | |
| | | 1H), 8.90 (d, J = 5.0 Hz, 1H), 8.48 | | | | | | |
| | | (d, J = 8.9 Hz, 1H), 8.33 (s, 1H), 8.06 | | | | | | |
| | | (d, J = 8.3 Hz, 1H), 7.83 (dd, J = 7.4, | | | | | | |
| | | 4.9 Hz, 1H), 7.36-7.12 (m, 2H), 6.96 | | | | | | |
| | | (d, J = 10.8 Hz, 1H), 4.16 (q, J = 6.6 | | | | | | |
| | | Hz, 2H), 3.26 (d, J = 3.9 Hz, 3H), | | | | | | |
| | | 1.36 (t, J = 6.5 Hz, 3H). | | | | | | |
| 146 | 2HCl | 1H NMR (300 MHz, CDCl3) δ 9.79 (d, | DMSO | 99 | Method N | 405 | 1.56 | Method D |
| | | J = 1.3 Hz, 1H), 8.93-8.79 (m, 1H), | | | DABCO as | (M + 1) | | |
| | | 8.70 (dd, J = 4.8, 1.7 Hz, 1H), 8.06- | | | additive a | | | |
| | | 7.76 (m, 3H), 7.42 (dd, J = 8.0, 4.0 | | | 80° C. | | | |
| | | Hz, 1H), 7.10-6.90 (m, 2H), 6.78- | | | | | | |
| | | 6.60 (m, 1H), 6.00 (d, J = 4.5 Hz, | | | | | | |
| | | 1H), 4.31-4.13 (m, 2H), 3.91-3.73 | | | | | | |
| | | (m, 2H), 3.48 (s, 3H), 3.35 (d, J = | | | | | | |
| | | 4.8 Hz, 3H). | | | | | | |
| 147 | 2HCl | 1H NMR (300 MHz, DMSO) δ 10.26 | DMSO | 99 | Method N | 468 | 1.58 | Method D |
| | | (s, 1H), 9.67 (s, 1H), 9.08 (d, J = 7.7 | | | | (M + 1) | | |
| | | Hz, 1H), 8.95 (d, J = 5.0 Hz, 1H), | | | | | | |
| | | 8.60 (d, J = 8.6 Hz, 1H), 8.49 (s, 1H), | | | | | | |
| | | 8.08 (d, J = 8.5 Hz, 1H), 8.01 (d, J = | | | | | | |
| | | 8.3 Hz, 1H), 7.94-7.81 (m, 1H), 7.59- | | | | | | |
| | | 7.32 (m, 3H), 7.08 (d, J = 7.9 Hz, | | | | | | |
| | | 1H), 4.58 (s, 2H), 3.71-3.53 (m, | | | | | | |
| | | 1H), 3.28 (d, J = 3.1 Hz, 3H), 1.88- | | | | | | |
| | | 1.49 (m, 5H), 1.42-0.99 (m, 5H). | | | | | | |
| 148 | 2 HCl | 1H NMR (300 MHz, DMSO) d 10.29 | DMSO | 95 | Method N | 346.5 | 2.09 | Method C |
| | | (s, 1H), 9.61 (s, 1H), 9.08-8.76 (m, | | | | (M + 1) | | |
| | | 3H), 8.36 (d, J = 9.0 Hz, 1H), 8.16 | | | | | | |
| | | (d, J = 8.8 Hz, 1H), 7.97-7.77 (m, | | | | | | |
| | | 1H), 7.53-7.37 (m, 3H), 7.02 (d, J = | | | | | | |
| | | 3.2 Hz, 1H), 3.29 (d, J = 4.4 Hz, 3H). | | | | | | |
| 149 | | 1H NMR (300 MHz, DMSO) d 9.57 (m, | DMSO | 100 | Method N | 429 | 1.57 | Method D |
| | | 1H), 8.80-8.50 (m, 2H), 8.25 (m, | | | | (M + 1) | | |
| | | 1H), 8.03 (dd, J = 8.7, 2.0 Hz, 1H), | | | | | | |
| | | 7.83 (dd, J = 8.7, 2.1 Hz, 1H), 7.57- | | | | | | |
| | | 7.14 (m, 4H), 6.96 (m, 1H), 4.88 (s, | | | | | | |
| | | 2H), 4.17 (q, J = 7.1 Hz, 2H), 3.41 | | | | | | |
| | | (d, J = 2.0 Hz, 6H), 1.20 (t, J = 7.1 | | | | | | |
| | | Hz, 3H). | | | | | | |
| 150 | 2HCl | 1H NMR (300 MHz, CD3OD) d 9.77 | CD3OD | 100 | Method N | 426 | 1.86 | Method C |
| | | (s, 1H), 9.36 (m, 1H), 9.11 (m, 1H), | | | K₂CO₃ and | (M + 1) | | |
| | | 8.70 (m, 1H), 8.36 (m, 1H), 8.23 (m, | | | DABCO as | | | |
| | | 1H), 8.13 (m, 1H), 7.60-7.32 (m, J = | | | base | | | |
| | | 4.8 Hz, 3H), 7.06 (m, 1H), 4.63 (m, | | | | | | |
| | | 2H), 3.47 (d, J = 5.8 Hz, 3H), 2.99- | | | | | | |
| | | 2.59 (m, 1H), 1.02-0.50 (m, 4H). | | | | | | |

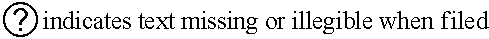

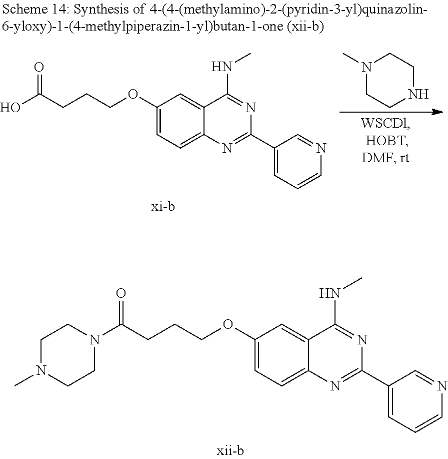

### Method P: 4-(4-(methylamino)-2-(pyridin-3-yl)quinazolin-6-yloxy)-1-(4-methylpiperazin-1-yl)butan-1-one trihydrochloride (xii-b)

In a 50 mL pear shaped flask was added 4-(4-(methylamino)-2-(pyridin-3-yl)quinazolin-6-yloxy)butanoic acid (synthesized following Scheme 13, method N, substituting methyl 4-bromobutanoate for 1-bromo-3-chloropropane and hydrolyzing the ester to the acid using NaOH/ethanol to give 4-(4-(methylamino)-2-(pyridin-3-yl)quinazolin-6-yloxy)butanoic acid) (0.180 g, 0.532 mmol), WSC-HCl (0.204 g, 1.064 mmol), and HOBt (0.163 g, 1.064 mmol) in DMF (5 ml) to give a yellow suspension. 1-Methyl piperazine (0.118 ml, 1.064 mmol) was added. The mixture was stirred overnight at room temperature and then diluted with water (10 mL) and extracted with ethyl acetate (2×10 mL). The combined organic layers were washed with water (1×20 mL) and brine (15 mL). The organic layer was dried over MgSO₄, filtered and concentrated down. The residue was purified via ISCO (amine silica gel, 3:1 to 0:1 Hex/EtOAc; 14 gm column). The product was converted to the HCl salt by treating with 4 M HCl-dioxane. The HCl salt was washed with ethyl acetate to give 15 mg of the desired product as a yellow solid in a 5.3% yield. LCMS m/z=421 (M+1) (Method C) (retention time=1.20 min). ¹H NMR (300 MHz, CD₃OD) δ 9.77 (d, J=1.8 Hz, 1H), 9.35 (d, J=8.3 Hz, 1H), 9.15-9.06 (m, 1H), 8.24 (dd, J=8.2, 5.6 Hz, 1H), 8.03 (d, J=9.2 Hz, 1H), 7.94 (d, J=2.5 Hz, 1H), 7.72 (dd, J=9.2, 2.5 Hz, 1H), 4.71 (d, J=11.2 Hz, 1H), 4.37-4.20 (m, 3H), 3.66-3.49 (m, 3H), 3.45 (s, 3H), 3.27-3.00 (m, 4H), 2.95 (s, 3H), 2.82-2.64 (m, 2H), 2.30-2.13 (m, 2H).

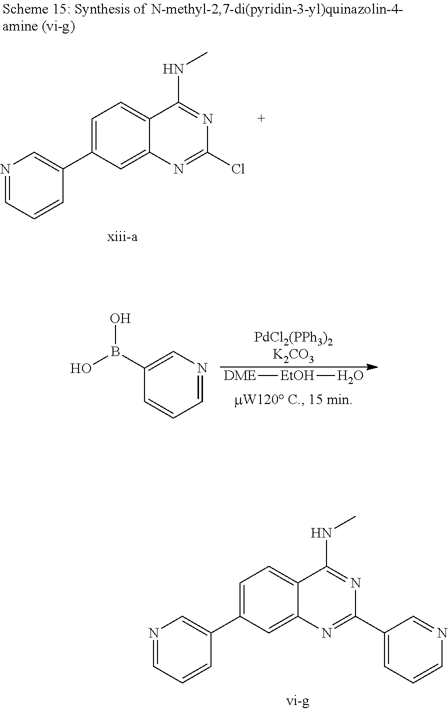

### Method Q: N-methyl-2,7-di(pyridin-3-yl)quinazolin-4-amine (vi-g)

In a 10 mL microwave vial was added 2-chloro-N-methyl-7-(pyridin-3-yl)quinazolin-4-amine (0.150 g, 0.554 mmol), pyridine-3-boronic acid (0.102 g, 0.831 mmol), trans-dichlorobis(triphenylphosphine)palladium (II) (Pd(PPh₃)₂Cl₂) (0.019 g, 0.028 mmol), and potassium carbonate (0.230 g, 1.662 mmol) in DME (3 ml), ethanol (1.286 ml), and water (0.429 ml) to give a yellow suspension. The vial was irradiated by microwave at 130° C. for 20 min under argon. Water (10 mL) was added to the reaction mixture and extracted with ethyl acetate (2×10 mL). The organic layers were combined and washed with brine (1×15 mL) and then dried over MgSO₄, filtered and concentrated. The residue was purified via ISCO (silica gel, 1:0 to 9:1 CH₂Cl₂/methanol; 12 gm Gold column). The fractions were collected to give 0.138 g of the desired product as the free base. The free base was converted to the HCl salt by addition of 4 M HCl-dioxane and recrystallized from ethanol/water to give 103 mg of the desired product as the HCl salt (a pale brown powder) in a 44% yield. LCMS m/z=314 (M+1) (Method D) (retention time=1.13 min) ¹H NMR (300 MHz, DMSO) δ 9.97 (s, 1H), 9.67 (s, 1H), 9.27 (s, 1H), 9.14 (d, J=7.2 Hz, 1H), 8.96 (d, J=4.6 Hz, 1H), 8.86 (d, J=4.8 Hz, 1H), 8.79-8.60 (m, 2H), 8.54 (s, 1H), 8.16 (d, J=8.6 Hz, 1H), 8.04-7.84 (m, 2H), 3.26 (d, J=4.0 Hz, 3H).

The compounds in the following table were prepared in a manner analogous to that described in Scheme 15 substituting with appropriate boronic acid or boronic ester.

TABLE 4

| | | | | | | | Puri- | Method | | Re- | |
|---|---|---|---|---|---|---|---|---|---|---|---|
| | | | | | | | ty | of | | ten- | LCMS |
| Num- | Starting | Starting | | Salt | | ¹H NMR | per- | Cou- | | tion | Meth- |
| ber | Material 1 | Material 2 | Product | type | ¹H NMR | Solvent | cent | pling | LCMS | Time | od |
| 151 | 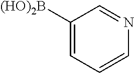 | 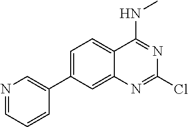 | 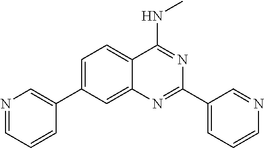 [SMILES: CNc1nc(-c2cccnc2)nc2cc(-c3cccnc3)ccc12] | 3HCl | 1H NMR (300 MHz, DMSO) δ 9.97 (s, 1H), 9.67 (s, 1 H), 9.27 (s, 1H), 9.14 (d, J = 7.2 Hz, 1H), 8.96 (d, J = 4.6 Hz, 1H), 8.86 (d, J = 4.8 Hz, 1H), 8.79-8.60 (m, 2H), | DMSO | 99 | Method Q | 314 (M + 1) | 1.13 | Meth- od D |
| | | | | | 8.54 (s, 1H), 8.16 | | | | | | |
| | | | | | (d, J = 8.6 Hz, 1H), | | | | | | |
| | | | | | 8.04-7.84 (m, 2H), | | | | | | |
| | | | | | 3.26 (d, J = 4.0 | | | | | | |
| | | | | | Hz, 3H). | | | | | | |
| 152 | 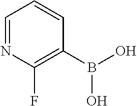 | 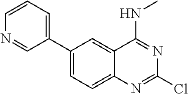 | 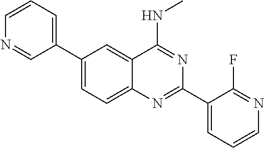 [SMILES: CNc1nc(-c2cccnc2F)nc2ccc(-c3cccnc3)cc12] | 3HCl | 1H NMR (300 MHz, DMSO) δ 10.97 (s, 1H), 9.56-9.35 (m, 2H), 9.04- 8.81 (m, 2H), 8.66 (t, J = 7.7 Hz, 1H), 8.61-8.45 (m, 2H), 8.15 (d, J = 8.6 | DMSO | 99 | Method Q | 332 (M + 1) | 1.07 | Meth- od D |
| | | | | | Hz, 1H), 8.10-7.96 | | | | | | |
| | | | | | (m, 1H), 7.77- | | | | | | |
| | | | | | 7.58 (m, 1H), 3.25 | | | | | | |
| | | | | | (d, J = 4.1 Hz, 3H). | | | | | | |
| 153 | 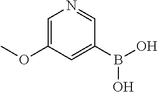 | 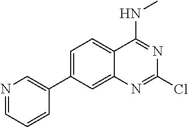 | 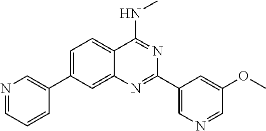 [SMILES: CNc1nc(-c2cncc(OC)c2)nc2cc(-c3cccnc3)ccc12] | 3HCl | 1H NMR (300 MHz, DMSO) δ 10.16 (s, 1H), 9.35-9.17 (m, 2H), 8.86 (d, J = 4.6 Hz, 1H), 8.80-8.51 (m, 5H), 8.17 (d, J = 8.7 Hz, 1H), 7.92 (dd, J = | DMSO | 99 | Method Q | 344 (M + 1) | 1.21 | Meth- od D |
| | | | | | 8.0, 5.2 Hz, 1H), | | | | | | |
| | | | | | 4.02 (s, 3H), 3.28 | | | | | | |
| | | | | | (d, J = 4.1 Hz, 3H). | | | | | | |
| 154 | 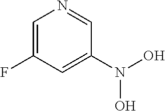 | 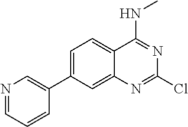 | 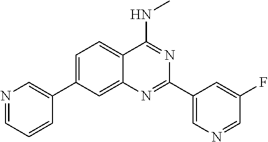 [SMILES: CNc1nc(-c2cncc(F)c2)nc2cc(-c3cccnc3)ccc12] | 3HCl | 1H NMR (300 MHz, DMSO) δ 9.65- 9.38 (m, 2H), 9.21 (s, 1H), 8.87- 8.76 (m, 2H), 8.66 (d, J = 9.8 Hz, 1H), 8.61-8.45 (m, 2H), 8.40 (s, 1H), 8.08 (d, J = 8.5 Hz, 1H), | DMSO | 99 | Method Q | 332 (M + 1) | 1.33 | Meth- od D |
| | | | | | 7.83 (dd, J = 7.7, | | | | | | |
| | | | | | 5.4 Hz, 1H), 3.23 | | | | | | |
| | | | | | (d, J = 4.3 Hz, 3H). | | | | | | |
| 155 | 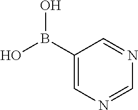 | 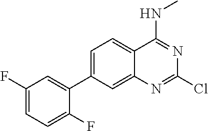 [SMILES: CNc1nc(Cl)nc2cc(-c3cc(F)ccc3F)ccc12] | 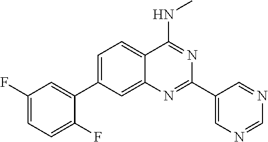 [SMILES: CNc1nc(-c2cncnc2)nc2cc(-c3cc(F)ccc3F)ccc12] | | 1H NMR (400 MHz, DMSO) δ 9.71 (s, 2H), 9.31 (s, 1H), 8.81-8.58 (m, 1H), 8.34 (d, J = 8.6 Hz, 1H), 8.03-7.95 (m, 1H), 7.81-7.71 (m, 1H), | DMSO | >98 | Method L | | | |
| | | | | | 7.63 (ddd, J = 9.2, | | | | | | |
| | | | | | 6.1, 3.2 Hz, 1H), | | | | | | |
| | | | | | 7.50-7.41 (m, 1H), | | | | | | |
| | | | | | 7.39-7.30 (m, | | | | | | |
| | | | | | 1H), 3.18 (d, J = | | | | | | |
| | | | | | 4.5 Hz, 3H). | | | | | | |
| 156 | 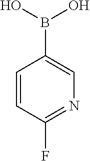 | 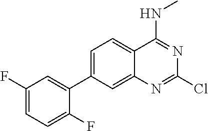 [SMILES: CNc1nc(Cl)nc2cc(-c3cc(F)ccc3F)ccc12] | 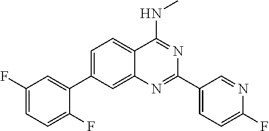 [SMILES: CNc1nc(-c2ccc(F)nc2)nc2cc(-c3cc(F)ccc3F)ccc12] | | 1H NMR (400 MHz, DMSO) δ 9.28 (s, 1H), 8.99-8.87 (m, 1H), 8.63- 8.52 (m, 1H), 8.32 (d, J = 8.3 Hz, 1H), 7.94 (s, 1H), 7.72 (d, J = 8.4 Hz, 1H), 7.66- | DMSO | >98 | Method L | | | |
| | | | | | 7.52 (m, 1H), | | | | | | |
| | | | | | 7.49-7.40 (m, | | | | | | |
| | | | | | 1H), 7.39-7.27 | | | | | | |
| | | | | | (m, 2H), 3.17 (d, | | | | | | |
| | | | | | J = 3.1 Hz, 3H). | | | | | | |
| 157 | 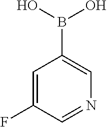 | 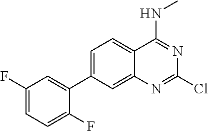 [SMILES: CNc1nc(Cl)nc2cc(-c3cc(F)ccc3F)ccc12] | 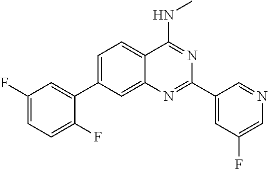 [SMILES: CNc1nc(-c2cncc(F)c2)nc2cc(-c3cc(F)ccc3F)ccc12] | | 1H NMR (400 MHz, DMSO) δ 9.59- 9.41 (m, 1H), 8.71 (d, J = 2.9 Hz, 1H), 8.68- 8.59 (m, 1H), 8.53 (ddd, J = 10.1, 2.9, 1.6 Hz, 1H), 8.33 (d, J = 8.6 Hz, 1H), 8.00-7.94 (m, 1H), | DMSO | >98 | Method L | | | |
| | | | | | 7.80-7.69 (m, 1H), | | | | | | |
| | | | | | 7.63 (ddd, J = 9.3, | | | | | | |
| | | | | | 6.1, 3.2 Hz, 1H), | | | | | | |
| | | | | | 7.50-7.41 (m, 1H), | | | | | | |
| | | | | | 7.40-7.30 (m, 1H), | | | | | | |
| | | | | | 3.18 (d, J = 4.5 | | | | | | |
| | | | | | Hz, 3H). | | | | | | |
| 158 | 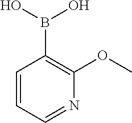 | 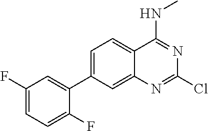 [SMILES: CNc1nc(Cl)nc2cc(-c3cc(F)ccc3F)ccc12] | 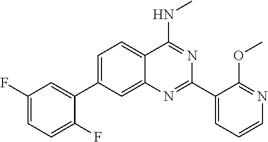 [SMILES: CNc1nc(-c2cccnc2OC)nc2cc(-c3cc(F)ccc3F)ccc12] | | 1H NMR (400 MHz, DMSO) δ 8.52- 8.41 (m, 1H), 8.32 (d, J = 8.6 Hz, 1H), 8.26 (dd, J = 4.9, 2.0 Hz, 1H), 8.04 (dd, J = 7.3, 2.0 Hz, 1H), 7.92-7.84 (m, 1H), | DMSO | >98 | Method L | | | |
| | | | | | 7.78-7.69 (m, 1H), | | | | | | |
| | | | | | 7.61 (ddd, J = 9.3, | | | | | | |
| | | | | | 6.1, 3.2 Hz, 1H), | | | | | | |
| | | | | | 7.50-7.40 (m, 1H), | | | | | | |
| | | | | | 7.40-7.29 (m, 1H), | | | | | | |
| | | | | | 7.11 (dd, J = 7.3, | | | | | | |
| | | | | | 4.9 Hz, 1H), 3.89 | | | | | | |
| | | | | | (s, 3H), 3.05 (d, | | | | | | |
| | | | | | J = 4.5 Hz, 3H). | | | | | | |
| 159 | 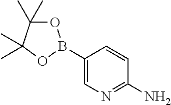 [SMILES: CC1(C)OB(c2ccc(N)nc2)OC1(C)C] | 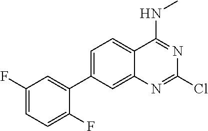 [SMILES: CNc1nc(Cl)nc2cc(-c3cc(F)ccc3F)ccc12] | 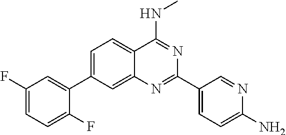 [SMILES: CNc1nc(-c2ccc(N)nc2)nc2cc(-c3cc(F)ccc3F)ccc12] | | 1H NMR (400 MHz, DMSO) δ 9.08 (dd, J = 2.3, 0.6 Hz, 1H), 8.41 (dd, J = 8.7, 2.3 Hz, 1H), 8.38-8.30 (m, 1H), 8.25 (d, J = 8.5 Hz, 1H), 7.87- | DMSO | >98 | Method L | | | |
| | | | | | 7.82 (m, 1H), 7.69- | | | | | | |
| | | | | | 7.56 (m, 2H), | | | | | | |
| | | | | | 7.52-7.39 (m, 1H), | | | | | | |
| | | | | | 7.38-7.23 (m, 1H), | | | | | | |
| | | | | | 6.53 (dd, J = 8.7, | | | | | | |
| | | | | | 0.6 Hz, 1H), 6.39 | | | | | | |
| | | | | | (s, 2H), 3.14 (d, | | | | | | |
| | | | | | J = 4.5 Hz, 3H). | | | | | | |
| 160 | 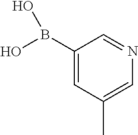 | 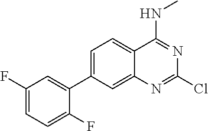 [SMILES: CNc1nc(Cl)nc2cc(-c3cc(F)ccc3F)ccc12] | 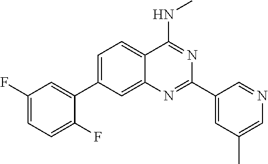 [SMILES: CNc1nc(-c2cncc(C)c2)nc2cc(-c3cc(F)ccc3F)ccc12] | | 1H NMR (400 MHz, DMSO) δ 9.46 (d, J = 1.6 Hz, 1H), 8.67-8.45 (m, 3H), 8.33 (d, J = 8.6 Hz, 1H), 8.01-7.91 (m, 1H), 7.80-7.69 (m, 1H), 7.64 (ddd, J = 9.3, | DMSO | >98 | Method L | | | |
| | | | | | 6.2, 3.2 Hz, 1H), | | | | | | |
| | | | | | 7.51-7.41 (m, 1H), | | | | | | |
| | | | | | 7.41-7.31 (m, 1H), | | | | | | |
| | | | | | 3.18 (d, J = 4.5 Hz, | | | | | | |
| | | | | | 3H), 2.43 (s, 3H). | | | | | | |
| 161 | 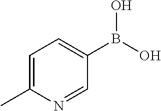 | 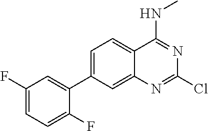 [SMILES: CNc1nc(Cl)nc2cc(-c3cc(F)ccc3F)ccc12] | 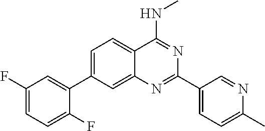 [SMILES: CNc1nc(-c2ccc(C)nc2)nc2cc(-c3cc(F)ccc3F)ccc12] | | 1H NMR (400 MHz, DMSO) δ 9.52 (d, J = 1.8 Hz, 1H), 8.67 (dd, J = 8.1, 2.2 Hz, 1H), 8.61-8.48 (m, 1H), 8.31 (d, J = 8.6 Hz, 1H), 7.95 (s, | DMSO | >98 | Method L | | | |
| | | | | | 1H), 7.77-7.68 (m, | | | | | | |
| | | | | | 1H), 7.63 (ddd, J = | | | | | | |
| | | | | | 9.3, 6.1, 3.2 Hz, | | | | | | |
| | | | | | 1H), 7.50-7.42 | | | | | | |
| | | | | | (m, 1H), 7.42-7.27 | | | | | | |
| | | | | | (m, 2H), 3.17 (d, | | | | | | |
| | | | | | J = 4.5 Hz, 3H), | | | | | | |
| | | | | | 2.56 (s, 3H). | | | | | | |
| 162 | 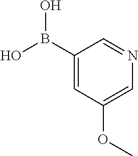 | 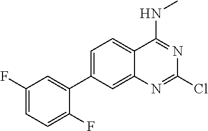 [SMILES: CNc1nc(Cl)nc2cc(-c3cc(F)ccc3F)ccc12] | 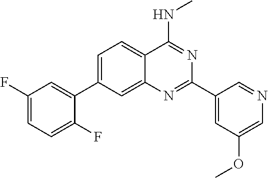 [SMILES: CNc1nc(-c2cncc(OC)c2)nc2cc(-c3cc(F)ccc3F)ccc12] | | 1H NMR (400 MHz, DMSO) δ 9.27 (d, J = 1.6 Hz, 1H), 8.63-8.53 (m, 1H), 8.42 (d, J = 2.9 Hz, 1H), 8.35- 8.28 (m, 2H), 8.00-7.96 (m, 1H), 7.77-7.69 (m, 1H), 7.64 (ddd, J = 9.3, 6.2, | DMSO | >98 | Method L | | | |
| | | | | | 3.2 Hz, 1H), | | | | | | |
| | | | | | 7.50-7.41 (m, 1H), | | | | | | |
| | | | | | 7.36 (ddd, J = | | | | | | |
| | | | | | 12.2, 8.4, 3.5 Hz, | | | | | | |
| | | | | | 1H), 3.95 (s, | | | | | | |
| | | | | | 3H), 3.18 (d, J = | | | | | | |
| | | | | | 4.4 Hz, 3H). | | | | | | |
| 163 | 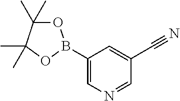 [SMILES: CC1(C)OB(c2cncc(C#N)c2)OC1(C)C] | 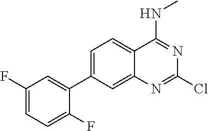 [SMILES: CNc1nc(Cl)nc2cc(-c3cc(F)ccc3F)ccc12] | 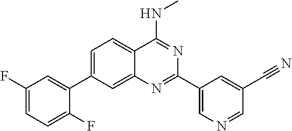 [SMILES: CNc1nc(-c2cncc(C#N)c2)nc2cc(-c3cc(F)ccc3F)ccc12] | HCl | 1H NMR (400 MHz, DMSO) δ 9.84 (brs, 2H), 9.26 (s, 2H), 8.53 (d, J = 7.9 Hz, 1H), 8.25 (s, 1H), 7.90 (d, J = 8.1 Hz, 1H), 7.75-7.57 (m, 1H), | DMSO | >98 | Method L | | | |
| | | | | | 7.55-7.45 (m, 1H), | | | | | | |
| | | | | | 7.45-7.34 (m, 1H), | | | | | | |
| | | | | | 3.31-3.22 (m, 3H). | | | | | | |
| 164 | 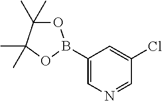 [SMILES: CC1(C)OB(c2cncc(Cl)c2)OC1(C)C] | 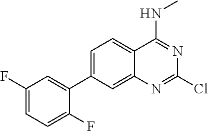 [SMILES: CNc1nc(Cl)nc2cc(-c3cc(F)ccc3F)ccc12] | 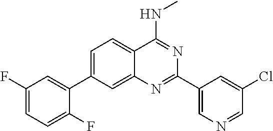 [SMILES: CNc1nc(-c2cncc(Cl)c2)nc2cc(-c3cc(F)ccc3F)ccc12] | | 1H NMR (400 MHz, DMSO) δ 9.58 (d, J = 1.7 Hz, 1H), 8.81-8.73 (m, 2H), 8.70-8.62 (m, 1H), 8.34 (d, J = 8.5 Hz, 1H), 8.05-7.98 | DMSO | >98 | Method L Temper- ature at 85° C. | | | |
| | | | | | (m, 1H), 7.79-7.73 | | | | | | |
| | | | | | (m, 1H), 7.64 | | | | | | |
| | | | | | (ddd, J = 9.2, 6.0, | | | | | | |
| | | | | | 3.2 Hz, 1H), 7.51- | | | | | | |
| | | | | | 7.41 (m, 1H), 7.41- | | | | | | |
| | | | | | 7.32 (m, 1H), 3.18 | | | | | | |
| | | | | | (d, J = 4.4 Hz, 3H). | | | | | | |
| 165 | 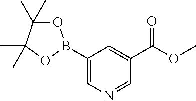 | 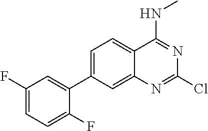 [SMILES: CNc1nc(Cl)nc2cc(-c3cc(F)ccc3F)ccc12] | 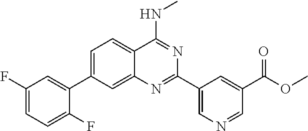 [SMILES: CNc1nc(-c2cncc(C(=O)OC)c2)nc2cc(-c3cc(F)ccc3F)ccc12] | | 1H NMR (400 MHz, DMSO) δ 9.84 (d, J = 2.1 Hz, 1H), 9.24 (t, J = 2.1 Hz, 1H), 9.19 (d, J = 2.1 Hz, 1H), 8.70-8.63 (m, 1H), 8.34 (d, | DMSO | >98 | Method L in Di- oxane- MeOH Temper- ature at 70° C. | | | |
| | | | | | J = 8.6 Hz, 1H), | | | | | | |
| | | | | | 8.02 (s, 1H), 7.83- | | | | | | |
| | | | | | 7.71 (m, 1H), 7.65 | | | | | | |
| | | | | | (ddd, J = 9.3, 6.2, | | | | | | |
| | | | | | 3.2 Hz, 1H), 7.51- | | | | | | |
| | | | | | 7.41 (m, 1H), 7.41- | | | | | | |
| | | | | | 7.30 (m, 1H), 3.96 | | | | | | |
| | | | | | (s, 3H), 3.19 (d, | | | | | | |
| | | | | | J = 4.3 Hz, 3H). | | | | | | |

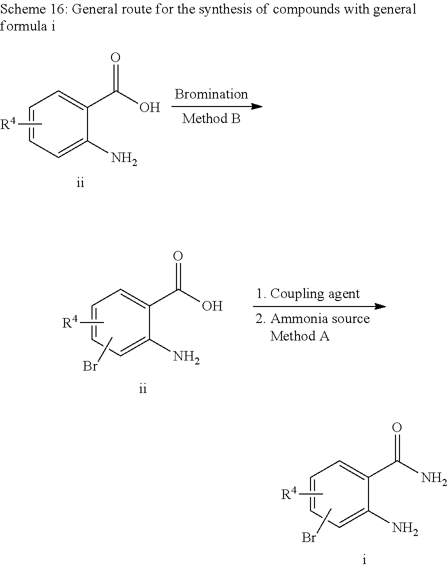

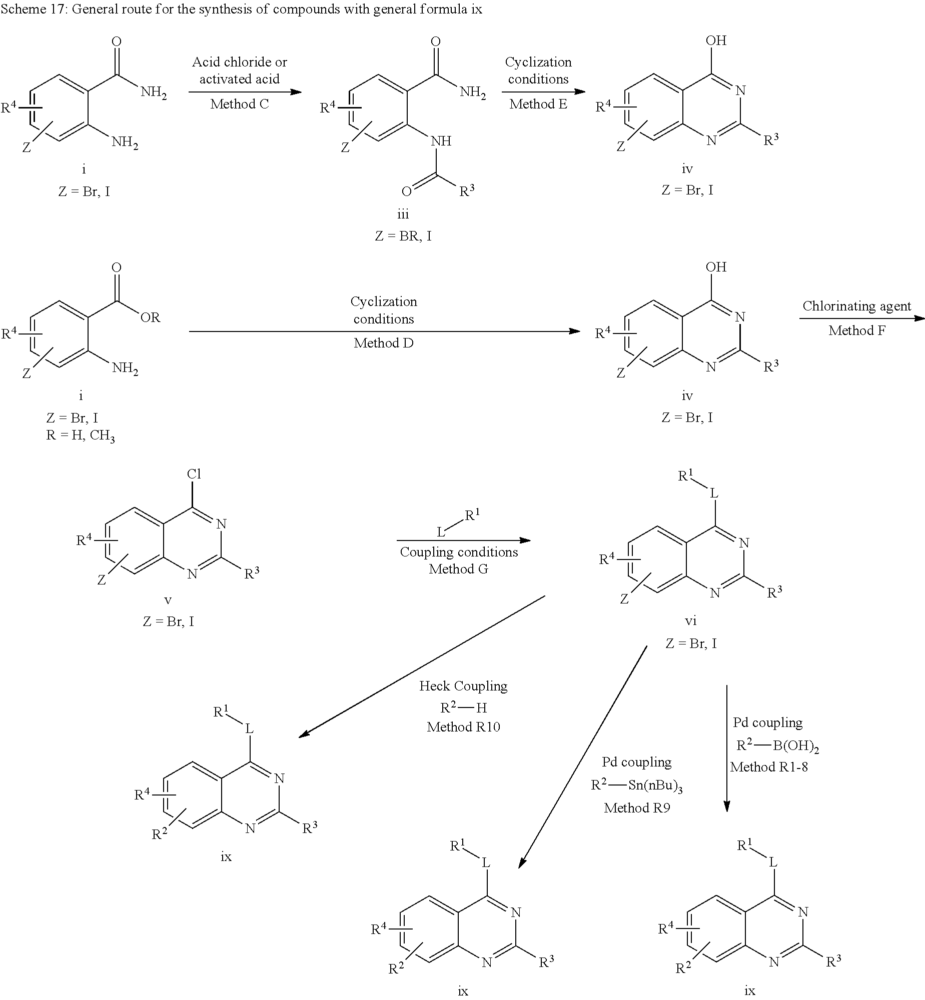

### Method C for Coupling Condition:

### C1: CH₂Cl₂/TEA

### C2: Pyridine/THF

### Method F for Chlorinating Conditions

### F1: SOCl₂/DMF/75° C.

### F2: POCl₃/Δ

### F3: POCl₃/Toluene/100° C.

### F4: PBr₃/CH₂Cl₂/DMF/60° C.

### Method G for Coupling Conditions

G1: i-PrOH/0.1 N HCl/85-100° C.

### G2: NaH/DMF

### G3: K₂CO₃/DMF/60° C.

### G4: THF/rt

### G5: DIPEA/DMA/50° C.

G6: iP2rNEt, dioxane reflux

### G7: DIPEA/THF/50° C.

### Method H for Coupling Conditions

H1: Pd₂(dba)₃/Xantphos/Cs₂CO₃/Dioxane/85-100° C.
H2: Pd₂(dba)₃/BINAP/NaO^{t}Bu/Dioxane/60° C.

### Method R for Coupling Conditions

### R1: Pd(PPh₃)₂Cl₂/K₂CO₃/Dioxane-H₂O

### R2: Pd₂(APhos)₂Cl₂/K₃PO₄/Dioxane-H₂O

### R3: Pd(PPh₃)₄/K₃PO₄/Dioxane-H₂O

### R4: Pd(dppf)Cl₂-CH₂Cl₂/K₃PO₄/Dioxane-H₂O

### R5: Pd(OAc)₂/S-Phos/K₃PO₄/Dioxane-H₂O

### R6: Pd(dppf)Cl₂-CH₂Cl₂/Na₂CO₃/Dioxane-H₂O

### R7: Pd(PPh₃)₂Cl₂/K₂CO₃/DME-EtOH-H₂O/microwave, 120° C.

### R8: Pd₂(APhos)₂Cl₂/K₃PO₄/Dioxane-H₂O/microwave, 110° C.

### R9: Pd(PPh₃)₄/K₃PO₄/Dioxane-H₂O/Stannane

### R10: Pd(OAc)₂/Cs₂CO₃/PPh₃/CuI/DMF/110° C.

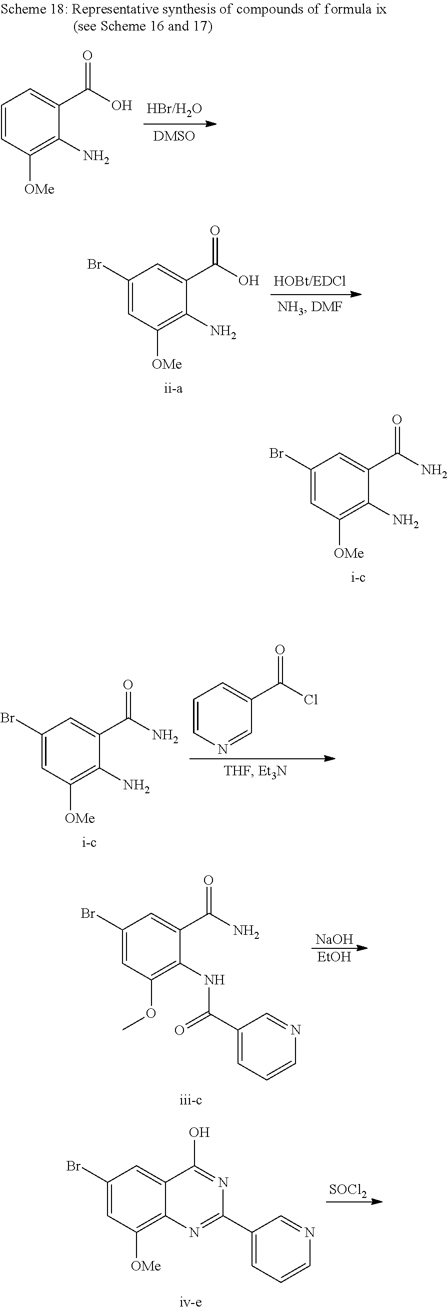

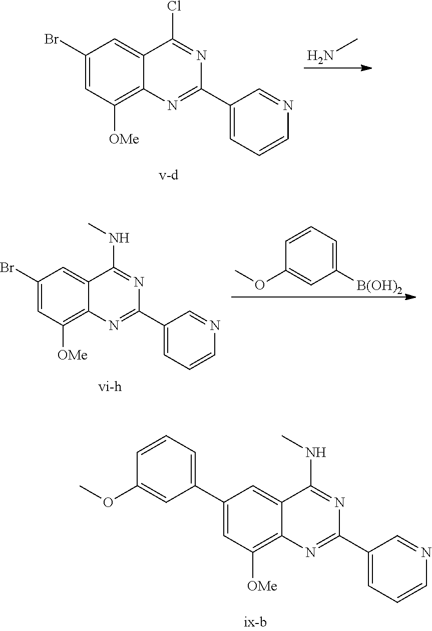

### Method B: 2-Amino-5-bromo-3-methoxybenzoic acid (ii-a)

To the solution of 2-amino-3-methoxybenzoic acid (10.0 g, 60 mmol) in DMSO (80 mL) was added HBr (33% in HOAc, 40 mL) dropwise. The resulting solution was stirred overnight and then poured into water (600 mL). The precipitate was collected to give the target product, 2-amino-5-bromo-3-methoxybenzoic acid, 14.1 g in a yield of 96%. LCMS m/z=246.0, 248.0 (M+1) (method B) (Retention time=1.159 min)

### Method A: 2-Amino-5-bromo-3-methoxybenzamide (1-c)

To a solution of 2-amino-5-bromo-3-methoxybenzoic acid (10.0 g, 40.6 mmol) and HOBt (6.04 g, 44 7 mmol) in DMF (300 mL) was added EDCI (8.57 g, 44.7 mmol). The resulting solution was stirred at room temperature for 2 h. NH₄OH (28%, 30 mL) was added dropwise under cooling in an ice-water bath. The mixture was stirred at room temperature for another 16 h and poured into water (2 L). The precipitate was collected to give the product, 2-amino-5-bromo-3-methoxybenzamide, 9.10 g with yield of 91%. LCMS m/z=245.0, 247.0 (M+1) (method B) (Retention time=1.415 min)

### Method C1: N-(4-bromo-2-carbamoyl-6-methoxyphenyl)nicotinamide (iii-c)

2-amino-5-bromo-3-methoxybenzamide (6.00 g, 24 5 mmol) was dissolved in CH₂Cl₂ (300 mL), and Et₃N (4.95 g, 49.0 mmol) was added to the solution. Nicotinoyl chloride (5.20 g, 36.7 mmol) was added in portions to the above mixture. The resulting solution was stirred overnight and then the volatiles were removed in vacuo to give the desired product, N-(4-bromo-2-carbamoyl-6-methoxyphenyl)nicotinamide, which was used directly in the next step without further purification. LCMS m/z=350.0 (M+1) (method B) (Retention time=1.264 min)

### Method E: 6-bromo-8-methoxy-2-(pyridin-3-yl)quinazolin-4-ol (iv-e)

The crude material, N-(4-bromo-2-carbamoyl-6-methoxyphenyl)nicotinamide, was dissolved in ethanol (300 ml), and NaOH (10.00 g, 250 mmol) was added in three portions. The resulting solution was stirred overnight. The volatiles were removed in vacuo and water (300 mL) was added to the residue. The mixture was neutralized with HCl (4N) to pH=6-7 and the precipitate was collected, washed with ethanol (3×100 mL) to give 3.50 g of the desired product, 6-bromo-8-methoxy-2-(pyridin-3-yl)quinazolin-4-ol (43% yield for two steps). LCMS m/z=332.0 (M+1) (method B) (Retention time=1.264 min).

### Method F1: 6-bromo-4-chloro-8-methoxy-2-(pyridin-3-yl)quinazoline (v-d)

To a mixture of 6-bromo-8-methoxy-2-(pyridin-3-yl)quinazolin-4-ol (6.00 g, 18 mmol) and DMF (0.5 mL) was added SOCl₂ (100 mL). The reaction mixture was stirred at 75° C. until the solution became clear. The volatiles were removed in vacuo and the crude precipitate was washed with ethyl acetate (100 mL). After drying, 6-bromo-4-chloro-8-methoxy-2-(pyridin-3-yl)quinazoline (6.20 g, 98%) was obtained. LCMS m/z=352 (M+1) (method A) (Retention time=1.70 min)

### Method G4: 6-bromo-8-methoxy-N-methyl-2-(pyridin-3-yl)quinazolin-4-amine (vi-h)

A solution of 6-bromo-4-chloro-8-methoxy-2-(pyridin-3-yl)quinazoline (6.20 g, 17.7 mmol) in THF (100 mL) was added dropwise to an aqueous solution of methylamine (50 mL) under ice cooling. The mixture was stirred at room temperature for 1 h. The volatiles were removed in vacuo. The crude product was washed with CH₂Cl₂ (100 mL) to give 6-bromo-8-methoxy-N-methyl-2-(pyridin-3-yl)quinazolin-4-amine (4.50 g, 74%). LCMS m/z=345 (M+1) (method B) (Retention time=1.55 m)

### Method R1: 8-methoxy-6-(3-methoxyphenyl)-N-methyl-2-(pyridin-3-yl)quinazolin-4-amine (ix-b)

A mixture of 6-bromo-8-methoxy-N-methyl-2-(pyridin-3-yl)quinazolin-4-amine (150 mg, 0.43 mmol), 3-methoxyphenylboronic acid (80 mg, 0.53 mmol, 1.2 eq), K₂CO₃ (425 mg, 1.31 mmol 3 eq), Pd(PPh₃)₂Cl₂ (15 mg, 0.02 mmol, 5% eq) in 30 ml of dioxane was stirred at reflux under N₂ atmosphere overnight. After cooling, the mixture was filtered and the filtrate was concentrated to give the crude product, which was purified by silica-gel column chromatography (dichloromethane: methanol=20:1) to afford 132 mg of 8-methoxy-6-(3-methoxyphenyl)-N-methyl-2-(pyridin-3-yl)quinazolin-4-amine as a yellow solid with a yield of 81%. LCMS m/z=372.9 (M+1) (Method A) (retention time=1.390 m) ¹H-NMR (400 MHz, DMSO-d₆): δ 9.63 (s, 1H), 8.77 (d, J=7.9 Hz, 1H), 8.67 (d, J=3.7 Hz, 1H), 8.50 (s, 1H), 8.11 (s, 1H), 7.54 (t, J=6.2 Hz, 2H), 7.48-7.40 (m, 3H), 7.01 (d, J=3.9 Hz, 1H), 4.07 (s, 3H), 3.88 (s, 3H), 3.17 (d, J=4.0 Hz, 3H).

### Method R2: 6-(6-methoxypyridin-3-yl)-N-methyl-2-(pyridine-3-yl)quinazoline-4-amine (ix-c) (This method is representative of method R3, R4 and R6 can be implemented in a similar way except for substitution of the appropriate catalyst and base)

To a 1 dram reaction vial were added 6-bromo-N-methyl-2-(pyridine-3-yl)quinazoline-4-amine (35 mg, 0.111 mmol), 6-methoxypyridin-3-ylboronic acid (20.4 mg, 0.133 mmol), Pd(APhos)₂Cl₂ (3.2 mg, 0.004 mmol) and potassium phosphate monohydrate (77 mg, 0.33 mmol) in a mixture of dioxane-water (9:1, 2 mL). The reaction mixture was heated to 90° C. for 14 h after which it was cooled to room temperature and diluted with water (5 mL). The resultant precipitate was collected by filtration and recrystallized from methanol to give 6-(6-methoxypyridin-3-yl)-N-methyl-2-(pyridine-3-yl)quinazoline-4-amine as a pale yellow solid (19.1 mg, 51%). LCMS m/z=344 (M+1) (Method C) (retention time=2.01 min). ¹H NMR (300 MHz, DMSO) δ 9.64 (d, J=1.3 Hz, 1H), 8.84-8.74 (m, 1H), 8.68 (dd, J=6.2, 1.7 Hz, 2H), 8.57 (d, J=1.6 Hz, 2H), 8.16 (ddd, J=14.4, 8.7, 2.2 Hz, 2H), 7.85 (d, J=8.7 Hz, 1H), 7.54 (dd, J=7.9, 4.8 Hz, 1H), 7.00 (d, J=8.7 Hz, 1H), 3.93 (s, 3H), 3.18 (d, J=4.3 Hz, 3H).

### Method R7: N-methyl-6-(2-methylbenzo[d]thiazol-5-yl)-2-(pyridin-3-yl)quinazolin-4-amine, 2HCl (ix-d)

To a 10 mL microwave vial were added 6-bromo-N-methyl-2-(pyridin-3-yl)quinazolin-4-amine (0.200 g, 0.635 mmol), 2-methylbenzo[d]thiazol-5-ylboronic acid (0.163 g, 0.844 mmol), trans-dichlorobis(triphenylphosphine)palladium (II) (Pd(PPh₃)₂Cl₂) (0.022 g, 0.032 mmol) and Potassium carbonate (0.439 g, 3.17 mmol) in DME (1.5 ml)-Water (0.643 ml)-Ethanol (0.429 ml) to give a brown suspension. The reaction mixture was then heated to 120° C. for 10 min by microwave irradiation. LC-MS analysis of the crude mixture showed the reaction was complete. Water (40 mL) was added to the reaction mixture, and the precipitate was filtered to give a brown solid. The residue was purified via ISCO (silica gel, 95:5 CH₂Cl₂/MeOH, 12 gm column). The fractions collected were concentrated and dried under vacuum to give a brown powder. To form the salt, the material was suspended in methanol prior to the addition of 4 M HCl in dioxane (0.55 mL). After stirring at ambient temperature for 2 h, the solvent was evaporated to give the desired product as a brown solid (204.1 mg, 0.45 mmol, 71%). LC-MS m/z=384.4 (M+1) (retention time=2.11) ¹H NMR (300 MHz, DMSO) δ 10.27 (s, 1H), 9.64 (d, J=2.1 Hz, 1H), 9.03 (d, J=7.6 Hz, 1H), 8.99-8.91 (m, 2H), 8.56 (d, J=1.3 Hz, 1H), 8.42 (dd, J=8.4, 1.4 Hz, 1H), 8.21 (d, J=8.7 Hz, 1H), 8.09-7.95 (m, 2H), 7.87 (dd, J=7.6, 5.2 Hz, 1H), 3.31 (d, J=4.4 Hz, 3H), 2.82 (s, 3H).

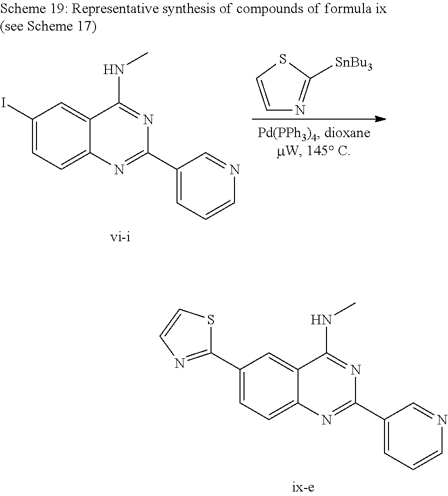

### Method R8: N-methyl-2-(pyridin-3-yl)-6-(thiazol-2-yl)quinazolin-4-amine, 2HCl (ix-e)

To a 10 mL microwave vial, under argon, were added 6-iodo-N-methyl-2-(pyridin-3-yl)quinazolin-4-amine (0.250 g, 0.690 mmol), 2-(tributylstannyl)thiazole (0.387 g, 1.035 mmol) and tetrakis(triphenylphosphine) palladium(0) (Pd(PPh₃)₄) (0.040 g, 0.035 mmol) in dioxane (2.5 ml) to give an orange suspension. The reaction mixture was then heated to 145° C. for 30 min by microwave irradiation. LC-MS analysis of the crude mixture showed the reaction was complete. The reaction mixture was diluted with water (40 mL) to give a brown precipitate. The residue was purified via ISCO (silica gel, 95:5 CH₂Cl₂/MeOH, 12 gm column). The fractions collected were concentrated and dried under vacuum to give an off-white solid. To form the salt, the material was suspended in methanol prior to the addition of 4 M HCl in dioxane. After stirring at ambient temperature for 2 h, the solvent was evaporated to give a yellow solid which was triturated with methanol (4 mL) and filtered to give the title compound (39.4 mg, 0.10 mmol, 15%). LC-MS m/z=320.4 (M+1) (retention time=1.88) ¹H NMR (300 MHz, DMSO) δ 10.14 (s, 1H), 9.65 (d, J=1.7 Hz, 1H), 9.11 (d, J=8.1 Hz, 1H), 9.02 (d, J=1.5 Hz, 1H), 8.95 (dd, J=5.1, 1.5 Hz, 1H), 8.52 (dd, J=8.8, 1.7 Hz, 1H), 8.19 (d, J=8.6 Hz, 1H), 8.02 (d, J=3.2 Hz, 1H), 7.97-7.87 (m, 2H), 3.27 (d, J=4.3 Hz, 3H).

### Method R9: 6-(2-amino-6-fluorophenyl)-N-methyl-2-(pyridin-3-yl)quinazolin-4-amine (ix-f)

A microwave vial was charged with 6-bromo-N-methyl-2-(pyridin-3-yl)quinazolin-4-amine (305 mg, 0.967 mmol), 2-amino-6-fluorophenylboronic acid (210 mg, 1.354 mmol, 1.40 equiv), Pd(APhos)₂Cl₂ (55 mg, 0.077 mmol, 8 mol %) and potassium phosphate monohydrate (617 mg, 2.91 mmol, 3.0 equiv). The mixture was suspended in dioxane/water (10:1, 5.5 mL), and the reaction was heated under microwave irradiation condition at 110° C. for 1.5 hours. The crude reaction mixture was cooled to room temperature and concentrated in vacuo. The residue was purified by chromatography on silica gel (petroleum ether: ethyl acetate 1:1). 286 mg (yield of 85.5%) of 6-(2-amino-6-fluorophenyl)-N-methyl-2-(pyridin-3-yl) quinazolin-4-amine was obtained as a light yellow solid. To a suspension of parent compound in methanol was added 4N HCl in methanol (ca. 4 mL) to give a clear solution. The solution was concentrated and recystallized from ethanol to give the HCl salt as pale yellow solid. LCMS m/z=346.1 (M+1) (Method B) (retention time=1.56 min) ¹H NMR (400 MHz, MeOD) δ 9.84 (d, J=1.6 Hz, 1H), 9.43 (d, J=8.4 Hz, 1H), 9.16 (d, J=4.8 Hz, 1H), 8.79 (s, 1H), 8.34-8.28 (m, 2H), 8.21 (d, J=8.4 Hz, 1H), 7.82-7.80 (m, 1H), 7.59-7.54 (m, 2H), 3.50 (s, 3H).

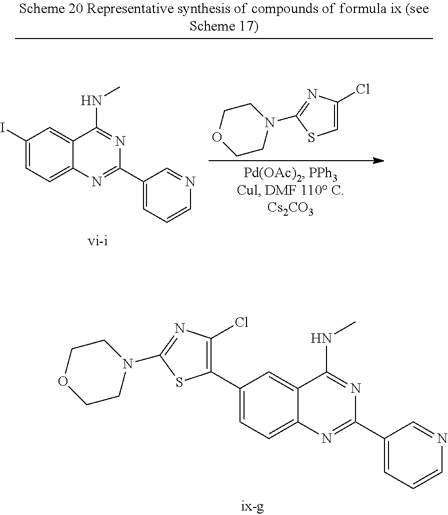 [SMILES: CNc1nc(-c2cccnc2)nc2ccc(I)cc12 ; Clc1csc(N2CCOCC2)n1 ; CNc1nc(-c2cccnc2)nc2ccc(-c3sc(N4CCOCC4)nc3Cl)cc12]

### Method R10: 6-(4-chloro-2-morphohnothiazol-5-yl)-N-methyl-2-(pyridin-3-yl)quinazolin-4-amine (ix-g)

In a 20 mL reaction vial were added 4-(4-chlorothiazol-2-yl)morpholine (237 mg, 1.160 mmol), palladium(II) acetate (3.72 mg, 0.017 mmol), cesium carbonate (567 mg, 1.740 mmol), triphenylphosphine (17.38 mg, 0.066 mmol), copper(I) iodide (7.89 mg, 0.041 mmol) and 6-iodo-N-methyl-2-(pyridin-3-yl)quinazolin-4-amine (300 mg, 0.828 mmol) in DMF (10 ml), and the mixture was heated at 110° C. overnight. After cooling to room temperature the reaction was poured into water (40 mL) and the resultant precipitate was collected by filtration, washed with water and methanol and dried to give the crude product. The product was recrystallized from methanol to afford 206 mg of 6-(4-chloro-2-morpholinothiazol-5-yl)-N-methyl-2-(pyridin-3-yl)quinazolin-4-amine as a brown solid (56.7%). LC-MS m/z=439 (M+1) (retention time=2.13) ¹H NMR (300 MHz, DMSO) δ 9.72 (s, 1H), 8.77 (d, J=7.6 Hz, 1H), 8.62 (d, J=4.2 Hz, 1H), 8.31 (d, J=1.6 Hz, 1H), 8.05 (dd, J=8.8, 1.8 Hz, 1H), 7.83 (d, J=8.7 Hz, 1H), 7.57 (s, 1H), 3.85-3.65 (m, 4H), 3.44 (dd, J=14.9, 10.5 Hz, 4H), 3.16 (d, J=4.2 Hz, 3H). NH was not observed.

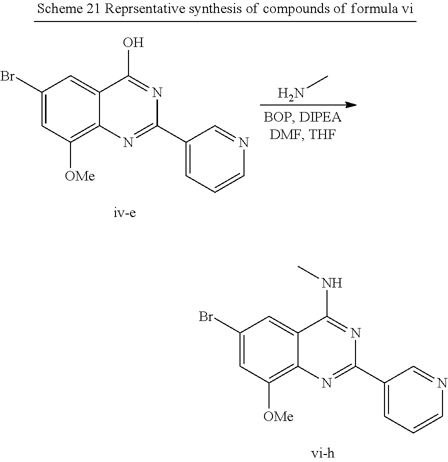

### Method S: 6-bromo-8-methoxy-N-methyl-2-(pyridin-3-yl)quinazolin-4-amine (vi-h)

6-bromo-8-methoxy-2-(pyridin-3-yl)quinazolin-4-ol (5.0 g), BOP (10 g 1.5 eq) and DIPEA (5.0 g 2.5 eq) were added to 90 mL of DMF/30 mL of THF and stirred at room temperature for 1 h. CH₃NH₂ (23 mL, 40% in H₂O) was added to the reaction and the mixture was allowed to stir at room temperature for 3 h. LCMS indicated that the reaction was completed. The reaction mixture was poured into water (300 mL). The precipitate was collected and suspended in dichloromethane (100 mL) with stirring for 3 h. After filtration, 6-bromo-8-methoxy-N-methyl-2-(pyridin-3-yl)quinazolin-4-amine was obtained (2.2 g). LCMS m/z=345 (M+1) (method B) (Retention time=1.55 min)

The compounds in the following table were prepared in a manner analogous to that described in Scheme 16-21, replacing methylamine with the appropriate amine and 6-methoxypyridin-3-ylboronic acid with the appropriate boronic acid/ester or stannane

TABLE 5

| Number | Starting Material 1 | Starting Material 2 | Product |
|---|---|---|---|
| 166 | 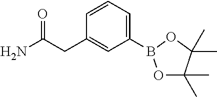 [SMILES: CC1(C)OB(c2cccc(CC(N)=O)c2)OC1(C)C] | 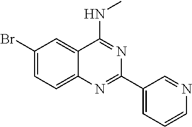 [SMILES: CNc1nc(-c2cccnc2)nc2ccc(Br)cc12] | 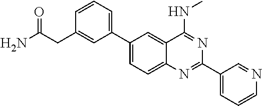 [SMILES: CNc1nc(-c2cccnc2)nc2ccc(-c3cccc(CC(N)=O)c3)cc12] |
| 167 | 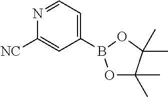 [SMILES: CC1(C)OB(c2ccnc(C#N)c2)OC1(C)C] | 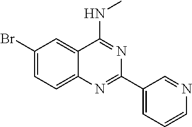 [SMILES: CNc1nc(-c2cccnc2)nc2ccc(Br)cc12] | 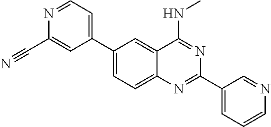 [SMILES: CNc1nc(-c2cccnc2)nc2ccc(-c3ccnc(C#N)c3)cc12] |
| 168 | 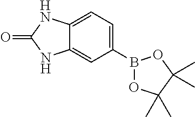 [SMILES: CC1(C)OB(c2ccc3[nH]c(=O)[nH]c3c2)OC1(C)C] | 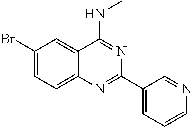 [SMILES: CNc1nc(-c2cccnc2)nc2ccc(Br)cc12] | 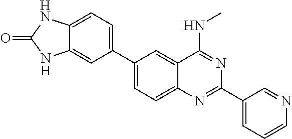 [SMILES: CNc1nc(-c2cccnc2)nc2ccc(-c3ccc4[nH]c(=O)[nH]c4c3)cc12] |
| 169 | 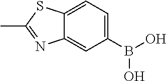 | 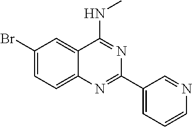 [SMILES: CNc1nc(-c2cccnc2)nc2ccc(Br)cc12] | 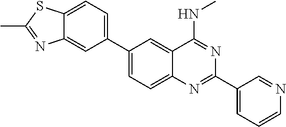 [SMILES: CNc1nc(-c2cccnc2)nc2ccc(-c3ccc4sc(C)nc4c3)cc12] |
| 170 | 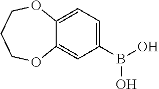 [SMILES: OB(O)c1ccc2c(c1)OCCCO2] | 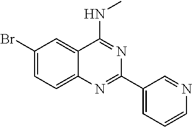 [SMILES: CNc1nc(-c2cccnc2)nc2ccc(Br)cc12] | 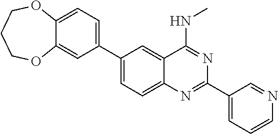 [SMILES: CNc1nc(-c2cccnc2)nc2ccc(-c3ccc4c(c3)OCCCO4)cc12] |
| 171 | 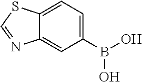 [SMILES: OB(O)c1ccc2scnc2c1] | 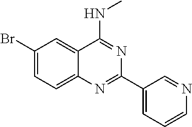 [SMILES: CNc1nc(-c2cccnc2)nc2ccc(Br)cc12] | 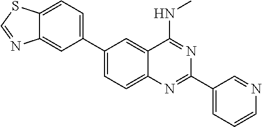 [SMILES: CNc1nc(-c2cccnc2)nc2ccc(-c3ccc4scnc4c3)cc12] |
| 172 | 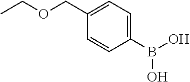 | 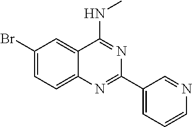 [SMILES: CNc1nc(-c2cccnc2)nc2ccc(Br)cc12] | 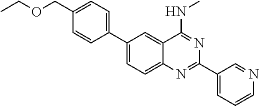 [SMILES: CCOCc1ccc(-c2ccc3nc(-c4cccnc4)nc(NC)c3c2)cc1] |
| 173 | 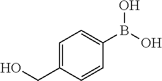 [SMILES: OCc1ccc(B(O)O)cc1] | 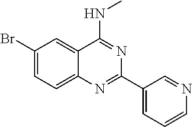 [SMILES: CNc1nc(-c2cccnc2)nc2ccc(Br)cc12] | 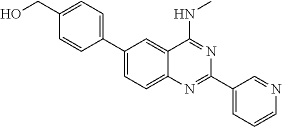 [SMILES: CNc1nc(-c2cccnc2)nc2ccc(-c3ccc(CO)cc3)cc12] |
| 174 | 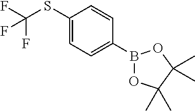 [SMILES: CC1(C)OB(c2ccc(SC(F)(F)F)cc2)OC1(C)C] | 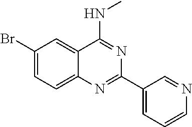 [SMILES: CNc1nc(-c2cccnc2)nc2ccc(Br)cc12] | 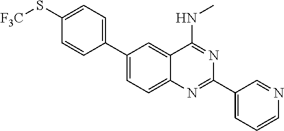 [SMILES: CNc1nc(-c2cccnc2)nc2ccc(-c3ccc(SC(F)(F)F)cc3)cc12] |
| 175 | 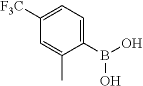 | 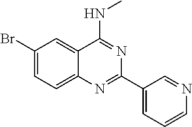 [SMILES: CNc1nc(-c2cccnc2)nc2ccc(Br)cc12] | 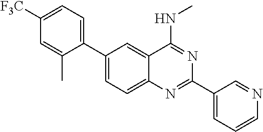 [SMILES: CNc1nc(-c2cccnc2)nc2ccc(-c3ccc(C(F)(F)F)cc3C)cc12] |
| 176 | 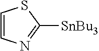 | 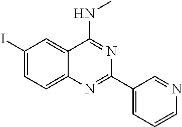 [SMILES: CNc1nc(-c2cccnc2)nc2ccc(I)cc12] | 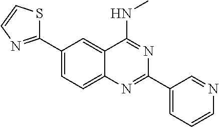 [SMILES: CNc1nc(-c2cccnc2)nc2ccc(-c3nccs3)cc12] |
| 177 | 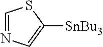 | 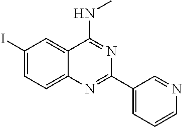 [SMILES: CNc1nc(-c2cccnc2)nc2ccc(I)cc12] | 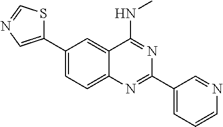 [SMILES: CNc1nc(-c2cccnc2)nc2ccc(-c3cncs3)cc12] |
| 178 | 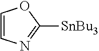 | 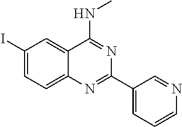 [SMILES: CNc1nc(-c2cccnc2)nc2ccc(I)cc12] | 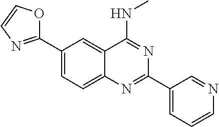 [SMILES: CNc1nc(-c2cccnc2)nc2ccc(-c3ncco3)cc12] |
| 179 | 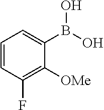 [SMILES: COc1c(F)cccc1B(O)O] | 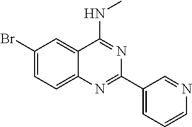 [SMILES: CNc1nc(-c2cccnc2)nc2ccc(Br)cc12] | 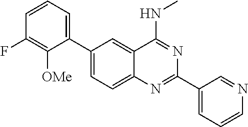 [SMILES: CNc1nc(-c2cccnc2)nc2ccc(-c3cccc(F)c3OC)cc12] |
| 180 | 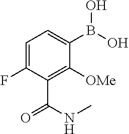 [SMILES: CNC(=O)c1c(F)ccc(B(O)O)c1OC] | 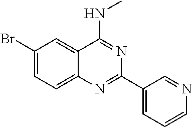 [SMILES: CNc1nc(-c2cccnc2)nc2ccc(Br)cc12] | 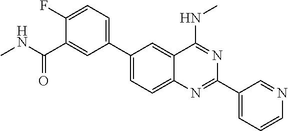 [SMILES: CNC(=O)c1cc(-c2ccc3nc(-c4cccnc4)nc(NC)c3c2)ccc1F] |
| 181 | 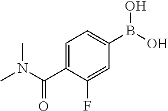 | 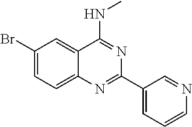 [SMILES: CNc1nc(-c2cccnc2)nc2ccc(Br)cc12] | 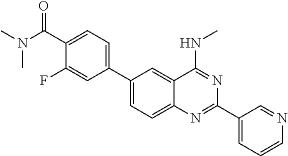 [SMILES: CNc1nc(-c2cccnc2)nc2ccc(-c3ccc(C(=O)N(C)C)c(F)c3)cc12] |
| 182 | 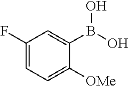 [SMILES: COc1ccc(F)cc1B(O)O] | 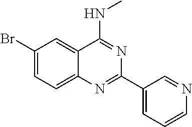 [SMILES: CNc1nc(-c2cccnc2)nc2ccc(Br)cc12] | 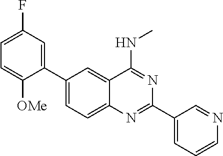 [SMILES: CNc1nc(-c2cccnc2)nc2ccc(-c3cc(F)ccc3OC)cc12] |
| 183 | 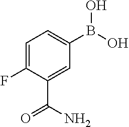 [SMILES: NC(=O)c1cc(B(O)O)ccc1F] | 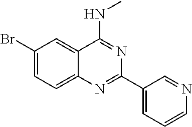 [SMILES: CNc1nc(-c2cccnc2)nc2ccc(Br)cc12] | 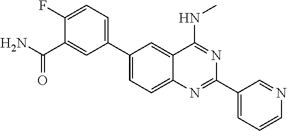 [SMILES: CNc1nc(-c2cccnc2)nc2ccc(-c3ccc(F)c(C(N)=O)c3)cc12] |
| 184 | 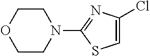 [SMILES: Clc1csc(N2CCOCC2)n1] | 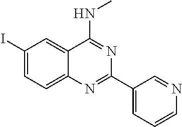 [SMILES: CNc1nc(-c2cccnc2)nc2ccc(I)cc12] | 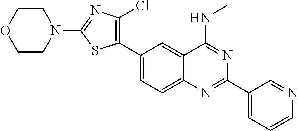 [SMILES: CNc1nc(-c2cccnc2)nc2ccc(-c3sc(N4CCOCC4)nc3Cl)cc12] |
| 185 | 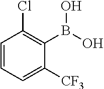 [SMILES: OB(O)c1c(Cl)cccc1C(F)(F)F] | 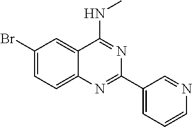 [SMILES: CNc1nc(-c2cccnc2)nc2ccc(Br)cc12] | 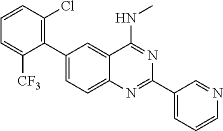 [SMILES: CNc1nc(-c2cccnc2)nc2ccc(-c3c(Cl)cccc3C(F)(F)F)cc12] |
| 186 | 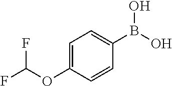 [SMILES: OB(O)c1ccc(OC(F)F)cc1] | 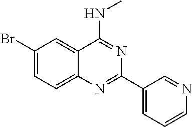 [SMILES: CNc1nc(-c2cccnc2)nc2ccc(Br)cc12] | 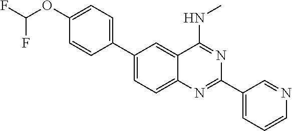 [SMILES: CNc1nc(-c2cccnc2)nc2ccc(-c3ccc(OC(F)F)cc3)cc12] |
| 187 | 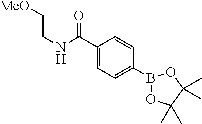 [SMILES: COCCNC(=O)c1ccc(B2OC(C)(C)C(C)(C)O2)cc1] | 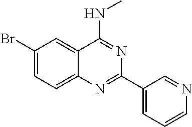 [SMILES: CNc1nc(-c2cccnc2)nc2ccc(Br)cc12] | 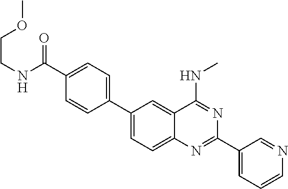 [SMILES: CNc1nc(-c2cccnc2)nc2ccc(-c3ccc(C(=O)NCCOC)cc3)cc12] |
| 188 | 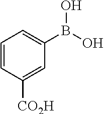 [SMILES: O=C(O)c1cccc(B(O)O)c1] | 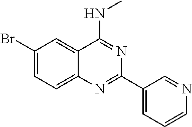 [SMILES: CNc1nc(-c2cccnc2)nc2ccc(Br)cc12] | 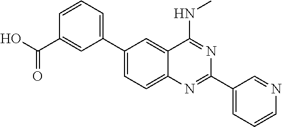 [SMILES: CNc1nc(-c2cccnc2)nc2ccc(-c3cccc(C(=O)O)c3)cc12] |
| 189 | 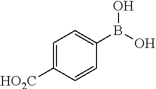 [SMILES: O=C(O)c1ccc(B(O)O)cc1] | 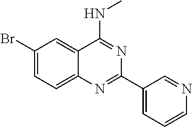 [SMILES: CNc1nc(-c2cccnc2)nc2ccc(Br)cc12] | 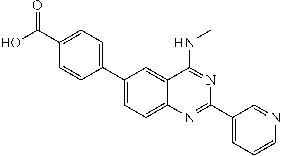 [SMILES: CNc1nc(-c2cccnc2)nc2ccc(-c3ccc(C(=O)O)cc3)cc12] |
| 190 | 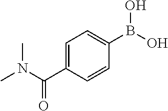 | 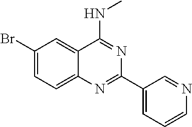 [SMILES: CNc1nc(-c2cccnc2)nc2ccc(Br)cc12] | 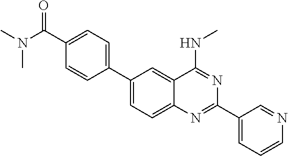 [SMILES: CNc1nc(-c2cccnc2)nc2ccc(-c3ccc(C(=O)N(C)C)cc3)cc12] |
| 191 | 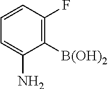 [SMILES: Nc1cccc(F)c1B(O)O] | 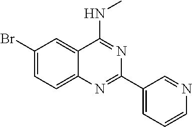 [SMILES: CNc1nc(-c2cccnc2)nc2ccc(Br)cc12] | 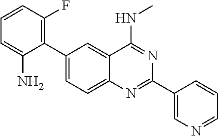 [SMILES: CNc1nc(-c2cccnc2)nc2ccc(-c3c(N)cccc3F)cc12] |
| 192 | 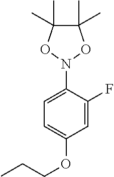 [SMILES: CCCOc1ccc(N2OC(C)(C)C(C)(C)O2)c(F)c1] | 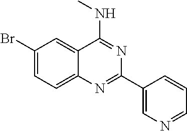 [SMILES: CNc1nc(-c2cccnc2)nc2ccc(Br)cc12] | 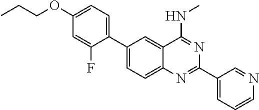 [SMILES: CCCOc1ccc(-c2ccc3nc(-c4cccnc4)nc(NC)c3c2)c(F)c1] |
| 193 | 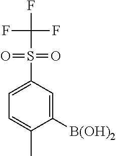 [SMILES: Cc1ccc(S(=O)(=O)C(F)(F)F)cc1B(O)O] | 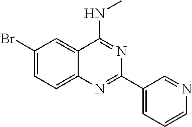 [SMILES: CNc1nc(-c2cccnc2)nc2ccc(Br)cc12] | 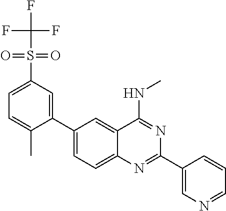 [SMILES: CNc1nc(-c2cccnc2)nc2ccc(-c3cc(S(=O)(=O)C(F)(F)F)ccc3C)cc12] |
| 194 | 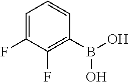 [SMILES: OB(O)c1cccc(F)c1F] | 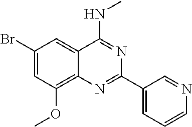 [SMILES: CNc1nc(-c2cccnc2)nc2c(OC)cc(Br)cc12] | 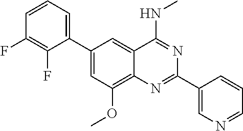 [SMILES: CNc1nc(-c2cccnc2)nc2c(OC)cc(-c3cccc(F)c3F)cc12] |
| 195 | 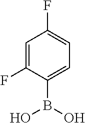 [SMILES: OB(O)c1ccc(F)cc1F] | 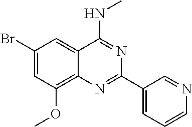 [SMILES: CNc1nc(-c2cccnc2)nc2c(OC)cc(Br)cc12] | 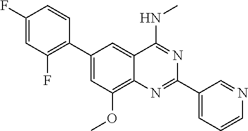 [SMILES: CNc1nc(-c2cccnc2)nc2c(OC)cc(-c3ccc(F)cc3F)cc12] |
| 196 | 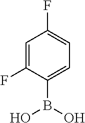 [SMILES: OB(O)c1ccc(F)cc1F] | 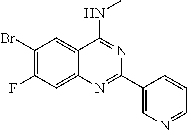 [SMILES: CNc1nc(-c2cccnc2)nc2cc(F)c(Br)cc12] | 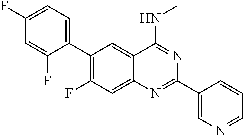 [SMILES: CNc1nc(-c2cccnc2)nc2cc(F)c(-c3ccc(F)cc3F)cc12] |
| 197 | 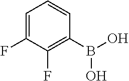 [SMILES: OB(O)c1cccc(F)c1F] | 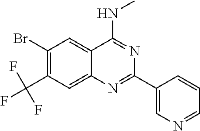 [SMILES: CNc1nc(-c2cccnc2)nc2cc(C(F)(F)F)c(Br)cc12] | 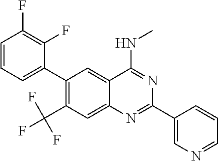 [SMILES: CNc1nc(-c2cccnc2)nc2cc(C(F)(F)F)c(-c3cccc(F)c3F)cc12] |
| 198 | 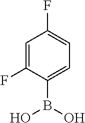 [SMILES: OB(O)c1ccc(F)cc1F] | 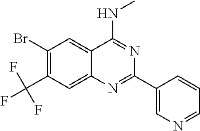 [SMILES: CNc1nc(-c2cccnc2)nc2cc(C(F)(F)F)c(Br)cc12] | 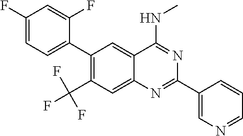 [SMILES: CNc1nc(-c2cccnc2)nc2cc(C(F)(F)F)c(-c3ccc(F)cc3F)cc12] |
| 199 | 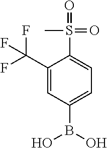 [SMILES: CS(=O)(=O)c1ccc(B(O)O)cc1C(F)(F)F] | 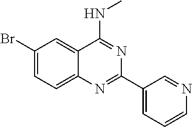 [SMILES: CNc1nc(-c2cccnc2)nc2ccc(Br)cc12] | 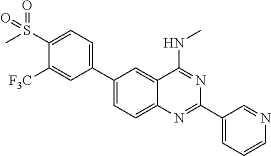 [SMILES: CNc1nc(-c2cccnc2)nc2ccc(-c3ccc(S(C)(=O)=O)c(C(F)(F)F)c3)cc12] |
| 200 | 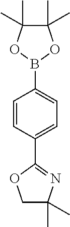 | 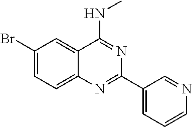 [SMILES: CNc1nc(-c2cccnc2)nc2ccc(Br)cc12] | 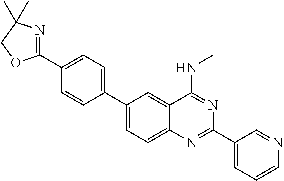 [SMILES: CNc1nc(-c2cccnc2)nc2ccc(-c3ccc(C4=NC(C)(C)CO4)cc3)cc12] |
| 201 | 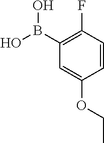 | 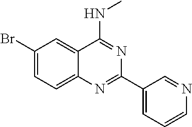 [SMILES: CNc1nc(-c2cccnc2)nc2ccc(Br)cc12] | 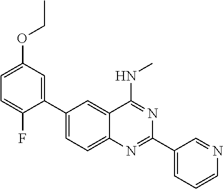 [SMILES: CCOc1ccc(F)c(-c2ccc3nc(-c4cccnc4)nc(NC)c3c2)c1] |
| 202 | 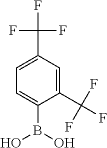 [SMILES: OB(O)c1ccc(C(F)(F)F)cc1C(F)(F)F] | 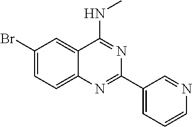 [SMILES: CNc1nc(-c2cccnc2)nc2ccc(Br)cc12] | 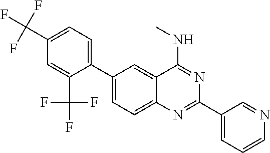 [SMILES: CNc1nc(-c2cccnc2)nc2ccc(-c3ccc(C(F)(F)F)cc3C(F)(F)F)cc12] |
| 203 | 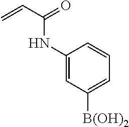 | 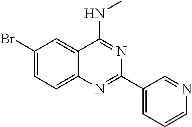 [SMILES: CNc1nc(-c2cccnc2)nc2ccc(Br)cc12] | 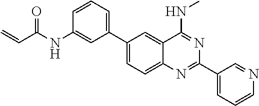 [SMILES: C=CC(=O)Nc1cccc(-c2ccc3nc(-c4cccnc4)nc(NC)c3c2)c1] |
| 204 | 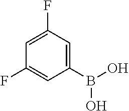 [SMILES: OB(O)c1cc(F)cc(F)c1] | 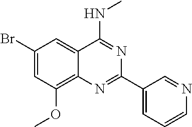 [SMILES: CNc1nc(-c2cccnc2)nc2c(OC)cc(Br)cc12] | 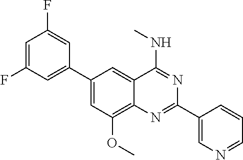 [SMILES: CNc1nc(-c2cccnc2)nc2c(OC)cc(-c3cc(F)cc(F)c3)cc12] |
| 205 | 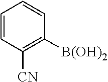 [SMILES: N#Cc1ccccc1B(O)O] | 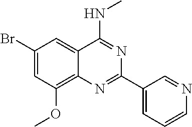 [SMILES: CNc1nc(-c2cccnc2)nc2c(OC)cc(Br)cc12] | 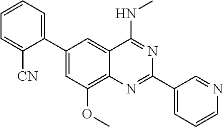 [SMILES: CNc1nc(-c2cccnc2)nc2c(OC)cc(-c3ccccc3C#N)cc12] |
| 206 | 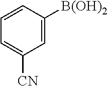 [SMILES: N#Cc1cccc(B(O)O)c1] | 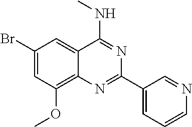 [SMILES: CNc1nc(-c2cccnc2)nc2c(OC)cc(Br)cc12] | 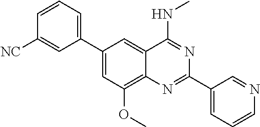 [SMILES: CNc1nc(-c2cccnc2)nc2c(OC)cc(-c3cccc(C#N)c3)cc12] |
| 207 | 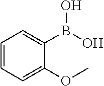 | 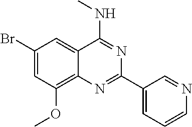 [SMILES: CNc1nc(-c2cccnc2)nc2c(OC)cc(Br)cc12] | 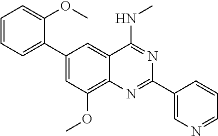 [SMILES: CNc1nc(-c2cccnc2)nc2c(OC)cc(-c3ccccc3OC)cc12] |
| 208 | 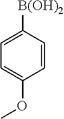 | 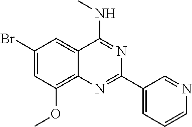 [SMILES: CNc1nc(-c2cccnc2)nc2c(OC)cc(Br)cc12] | 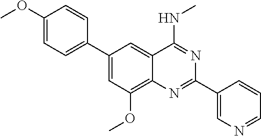 [SMILES: CNc1nc(-c2cccnc2)nc2c(OC)cc(-c3ccc(OC)cc3)cc12] |
| 209 | 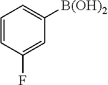 | 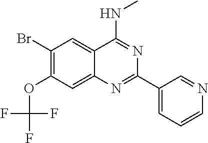 [SMILES: CNc1nc(-c2cccnc2)nc2cc(OC(F)(F)F)c(Br)cc12] | 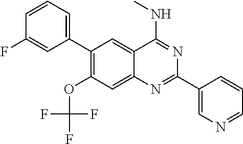 [SMILES: CNc1nc(-c2cccnc2)nc2cc(OC(F)(F)F)c(-c3cccc(F)c3)cc12] |
| 210 | 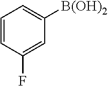 | 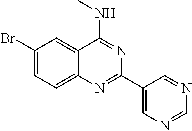 [SMILES: CNc1nc(-c2cncnc2)nc2ccc(Br)cc12] | 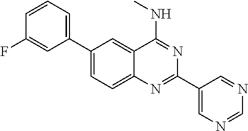 [SMILES: CNc1nc(-c2cncnc2)nc2ccc(-c3cccc(F)c3)cc12] |
| 211 | 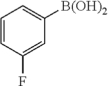 | 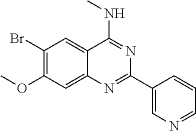 [SMILES: CNc1nc(-c2cccnc2)nc2cc(OC)c(Br)cc12] | 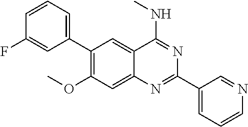 [SMILES: CNc1nc(-c2cccnc2)nc2cc(OC)c(-c3cccc(F)c3)cc12] |
| 212 | 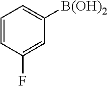 | 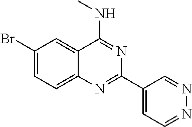 [SMILES: CNc1nc(-c2ccnnc2)nc2ccc(Br)cc12] | 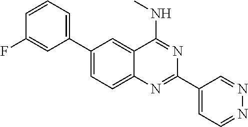 [SMILES: CNc1nc(-c2ccnnc2)nc2ccc(-c3cccc(F)c3)cc12] |
| 213 | 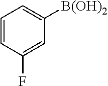 | 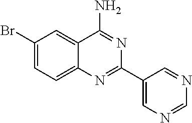 [SMILES: Nc1nc(-c2cncnc2)nc2ccc(Br)cc12] | 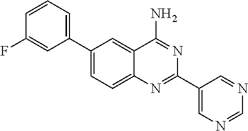 [SMILES: Nc1nc(-c2cncnc2)nc2ccc(-c3cccc(F)c3)cc12] |
| 214 | 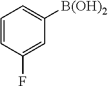 | 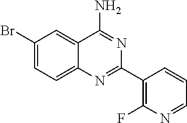 [SMILES: Nc1nc(-c2cccnc2F)nc2ccc(Br)cc12] | 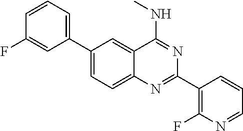 [SMILES: CNc1nc(-c2cccnc2F)nc2ccc(-c3cccc(F)c3)cc12] |
| 215 | 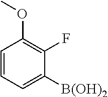 | 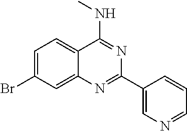 [SMILES: CNc1nc(-c2cccnc2)nc2cc(Br)ccc12] | 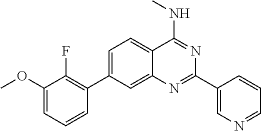 [SMILES: CNc1nc(-c2cccnc2)nc2cc(-c3cccc(OC)c3F)ccc12] |
| 216 | 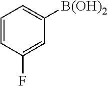 | 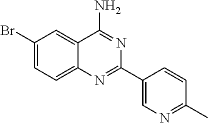 [SMILES: Cc1ccc(-c2nc(N)c3cc(Br)ccc3n2)cn1] | 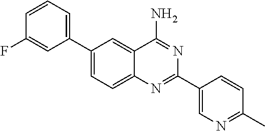 [SMILES: Cc1ccc(-c2nc(N)c3cc(-c4cccc(F)c4)ccc3n2)cn1] |
| 217 | 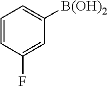 | 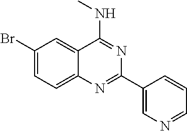 [SMILES: CNc1nc(-c2cccnc2)nc2ccc(Br)cc12] | 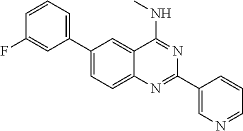 [SMILES: CNc1nc(-c2cccnc2)nc2ccc(-c3cccc(F)c3)cc12] |
| 218 | 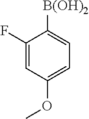 | 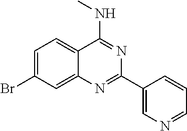 [SMILES: CNc1nc(-c2cccnc2)nc2cc(Br)ccc12] | 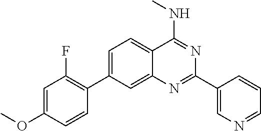 [SMILES: CNc1nc(-c2cccnc2)nc2cc(-c3ccc(OC)cc3F)ccc12] |
| 219 | 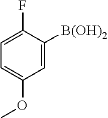 | 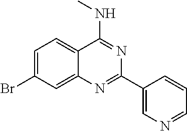 [SMILES: CNc1nc(-c2cccnc2)nc2cc(Br)ccc12] | 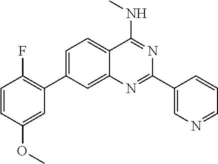 [SMILES: CNc1nc(-c2cccnc2)nc2cc(-c3cc(OC)ccc3F)ccc12] |
| 220 | 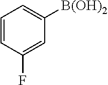 | 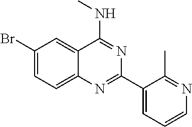 [SMILES: CNc1nc(-c2cccnc2C)nc2ccc(Br)cc12] | 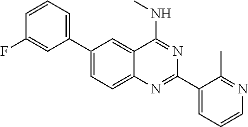 [SMILES: CNc1nc(-c2cccnc2C)nc2ccc(-c3cccc(F)c3)cc12] |
| 221 | 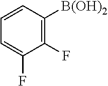 [SMILES: OB(O)c1cccc(F)c1F] | 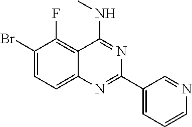 [SMILES: CNc1nc(-c2cccnc2)nc2ccc(Br)c(F)c12] | 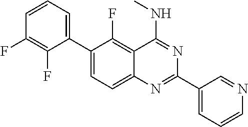 [SMILES: CNc1nc(-c2cccnc2)nc2ccc(-c3cccc(F)c3F)c(F)c12] |
| 222 | 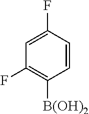 [SMILES: OB(O)c1ccc(F)cc1F] | 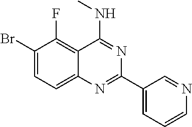 [SMILES: CNc1nc(-c2cccnc2)nc2ccc(Br)c(F)c12] | 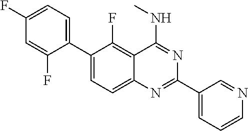 [SMILES: CNc1nc(-c2cccnc2)nc2ccc(-c3ccc(F)cc3F)c(F)c12] |
| 223 | 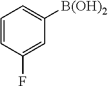 | 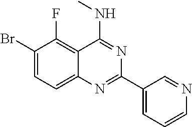 [SMILES: CNc1nc(-c2cccnc2)nc2ccc(Br)c(F)c12] | 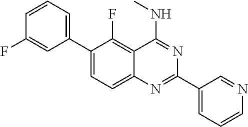 [SMILES: CNc1nc(-c2cccnc2)nc2ccc(-c3cccc(F)c3)c(F)c12] |
| 224 | 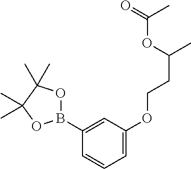 [SMILES: CC(=O)OC(C)CCOc1cccc(B2OC(C)(C)C(C)(C)O2)c1] | 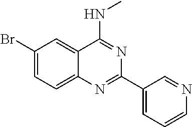 [SMILES: CNc1nc(-c2cccnc2)nc2ccc(Br)cc12] | 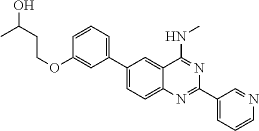 [SMILES: CNc1nc(-c2cccnc2)nc2ccc(-c3cccc(OCCC(C)O)c3)cc12] |
| 225 | 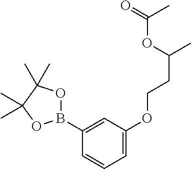 [SMILES: CC(=O)OC(C)CCOc1cccc(B2OC(C)(C)C(C)(C)O2)c1] | 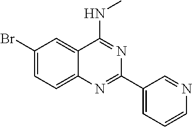 [SMILES: CNc1nc(-c2cccnc2)nc2ccc(Br)cc12] | 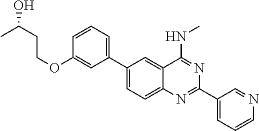 [SMILES: CNc1nc(-c2cccnc2)nc2ccc(-c3cccc(OCC[C@H](C)O)c3)cc12] |
| 226 | 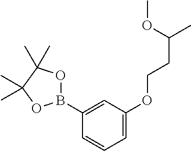 [SMILES: COC(C)CCOc1cccc(B2OC(C)(C)C(C)(C)O2)c1] | 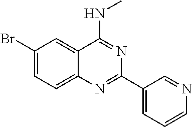 [SMILES: CNc1nc(-c2cccnc2)nc2ccc(Br)cc12] | 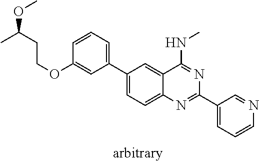 [SMILES: CNc1nc(-c2cccnc2)nc2ccc(-c3cccc(OCC[C@@H](C)OC)c3)cc12] |
| 227 | 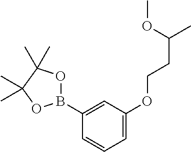 [SMILES: COC(C)CCOc1cccc(B2OC(C)(C)C(C)(C)O2)c1] | 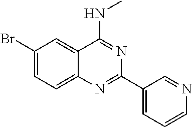 [SMILES: CNc1nc(-c2cccnc2)nc2ccc(Br)cc12] | 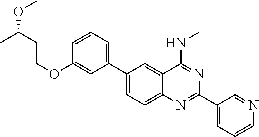 [SMILES: CNc1nc(-c2cccnc2)nc2ccc(-c3cccc(OCC[C@H](C)OC)c3)cc12] |
| 228 | 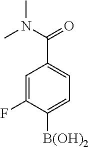 | 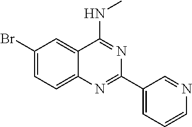 [SMILES: CNc1nc(-c2cccnc2)nc2ccc(Br)cc12] | 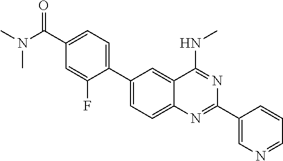 [SMILES: CNc1nc(-c2cccnc2)nc2ccc(-c3ccc(C(=O)N(C)C)cc3F)cc12] |
| 229 | 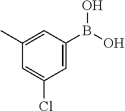 | 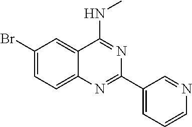 [SMILES: CNc1nc(-c2cccnc2)nc2ccc(Br)cc12] | 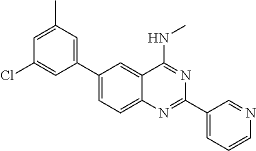 [SMILES: CNc1nc(-c2cccnc2)nc2ccc(-c3cc(C)cc(Cl)c3)cc12] |
| 230 | 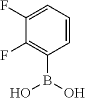 [SMILES: OB(O)c1cccc(F)c1F] | 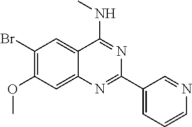 [SMILES: CNc1nc(-c2cccnc2)nc2cc(OC)c(Br)cc12] | 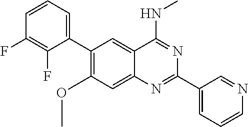 [SMILES: CNc1nc(-c2cccnc2)nc2cc(OC)c(-c3cccc(F)c3F)cc12] |
| 231 | 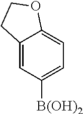 [SMILES: OB(O)c1ccc2c(c1)CCO2] | 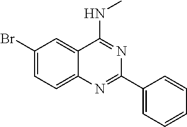 [SMILES: CNc1nc(-c2ccccc2)nc2ccc(Br)cc12] | 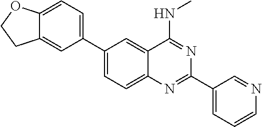 [SMILES: CNc1nc(-c2cccnc2)nc2ccc(-c3ccc4c(c3)CCO4)cc12] |
| 232 | 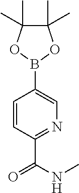 | 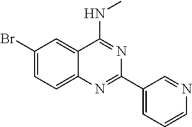 [SMILES: CNc1nc(-c2cccnc2)nc2ccc(Br)cc12] | 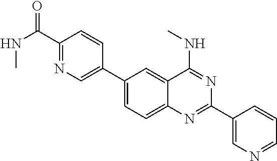 [SMILES: CNC(=O)c1ccc(-c2ccc3nc(-c4cccnc4)nc(NC)c3c2)cn1] |
| 233 | 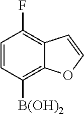 [SMILES: OB(O)c1ccc(F)c2ccoc12] | 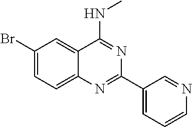 [SMILES: CNc1nc(-c2cccnc2)nc2ccc(Br)cc12] | 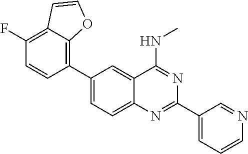 [SMILES: CNc1nc(-c2cccnc2)nc2ccc(-c3ccc(F)c4ccoc34)cc12] |
| 234 | 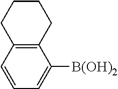 [SMILES: OB(O)c1cccc2c1CCCC2] | 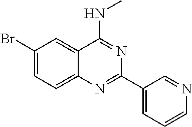 [SMILES: CNc1nc(-c2cccnc2)nc2ccc(Br)cc12] | 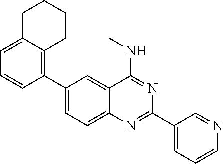 [SMILES: CNc1nc(-c2cccnc2)nc2ccc(-c3cccc4c3CCCC4)cc12] |
| 235 | 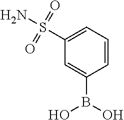 [SMILES: NS(=O)(=O)c1cccc(B(O)O)c1] | 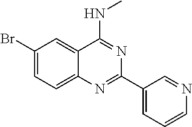 [SMILES: CNc1nc(-c2cccnc2)nc2ccc(Br)cc12] | 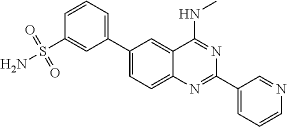 [SMILES: CNc1nc(-c2cccnc2)nc2ccc(-c3cccc(S(N)(=O)=O)c3)cc12] |
| 236 | 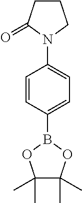 [SMILES: CC1(C)OB(c2ccc(N3CCCC3=O)cc2)OC1(C)C] | 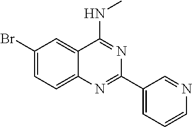 [SMILES: CNc1nc(-c2cccnc2)nc2ccc(Br)cc12] | 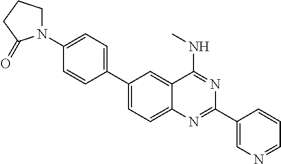 [SMILES: CNc1nc(-c2cccnc2)nc2ccc(-c3ccc(N4CCCC4=O)cc3)cc12] |
| 237 | 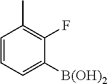 | 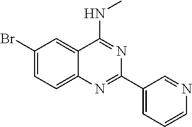 [SMILES: CNc1nc(-c2cccnc2)nc2ccc(Br)cc12] | 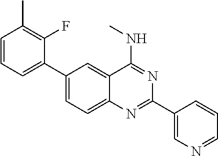 [SMILES: CNc1nc(-c2cccnc2)nc2ccc(-c3cccc(C)c3F)cc12] |
| 238 | 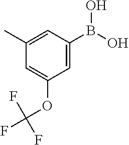 | 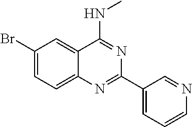 [SMILES: CNc1nc(-c2cccnc2)nc2ccc(Br)cc12] | 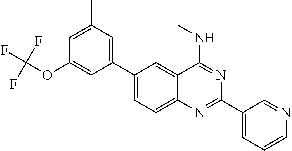 [SMILES: CNc1nc(-c2cccnc2)nc2ccc(-c3cc(C)cc(OC(F)(F)F)c3)cc12] |
| 239 | 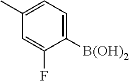 | 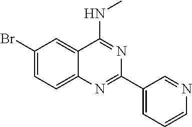 [SMILES: CNc1nc(-c2cccnc2)nc2ccc(Br)cc12] | 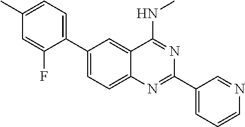 [SMILES: CNc1nc(-c2cccnc2)nc2ccc(-c3ccc(C)cc3F)cc12] |
| 240 | 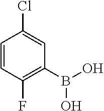 [SMILES: OB(O)c1cc(Cl)ccc1F] | 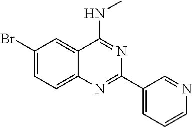 [SMILES: CNc1nc(-c2cccnc2)nc2ccc(Br)cc12] | 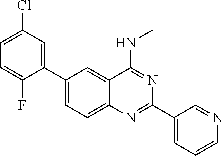 [SMILES: CNc1nc(-c2cccnc2)nc2ccc(-c3cc(Cl)ccc3F)cc12] |
| 241 | 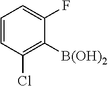 [SMILES: OB(O)c1c(F)cccc1Cl] | 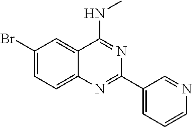 [SMILES: CNc1nc(-c2cccnc2)nc2ccc(Br)cc12] | 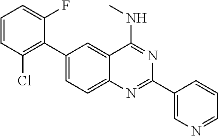 [SMILES: CNc1nc(-c2cccnc2)nc2ccc(-c3c(F)cccc3Cl)cc12] |
| 242 | 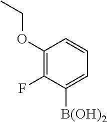 | 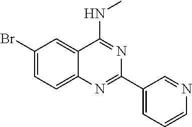 [SMILES: CNc1nc(-c2cccnc2)nc2ccc(Br)cc12] | 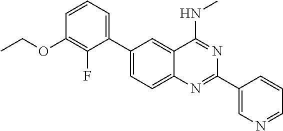 [SMILES: CCOc1cccc(-c2ccc3nc(-c4cccnc4)nc(NC)c3c2)c1F] |
| 243 | 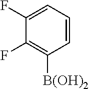 [SMILES: OB(O)c1cccc(F)c1F] | 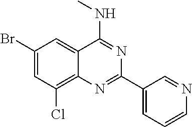 [SMILES: CNc1nc(-c2cccnc2)nc2c(Cl)cc(Br)cc12] | 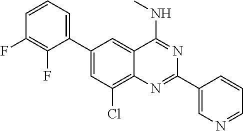 [SMILES: CNc1nc(-c2cccnc2)nc2c(Cl)cc(-c3cccc(F)c3F)cc12] |
| 244 | 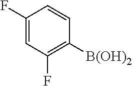 [SMILES: OB(O)c1ccc(F)cc1F] | 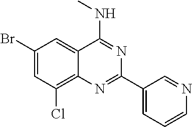 [SMILES: CNc1nc(-c2cccnc2)nc2c(Cl)cc(Br)cc12] | 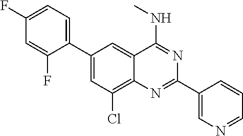 [SMILES: CNc1nc(-c2cccnc2)nc2c(Cl)cc(-c3ccc(F)cc3F)cc12] |
| 245 | 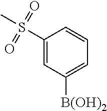 | 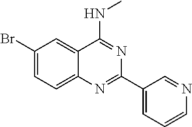 [SMILES: CNc1nc(-c2cccnc2)nc2ccc(Br)cc12] | 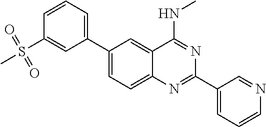 [SMILES: CNc1nc(-c2cccnc2)nc2ccc(-c3cccc(S(C)(=O)=O)c3)cc12] |
| 246 | 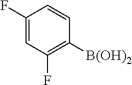 [SMILES: OB(O)c1ccc(F)cc1F] | 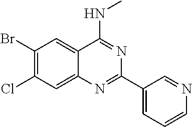 [SMILES: CNc1nc(-c2cccnc2)nc2cc(Cl)c(Br)cc12] | 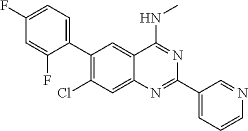 [SMILES: CNc1nc(-c2cccnc2)nc2cc(Cl)c(-c3ccc(F)cc3F)cc12] |
| 247 | 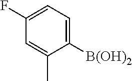 | 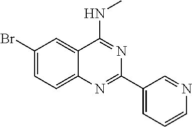 [SMILES: CNc1nc(-c2cccnc2)nc2ccc(Br)cc12] | 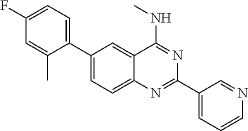 [SMILES: CNc1nc(-c2cccnc2)nc2ccc(-c3ccc(F)cc3C)cc12] |
| 248 | 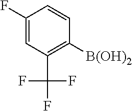 [SMILES: OB(O)c1ccc(F)cc1C(F)(F)F] | 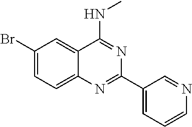 [SMILES: CNc1nc(-c2cccnc2)nc2ccc(Br)cc12] | 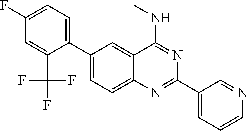 [SMILES: CNc1nc(-c2cccnc2)nc2ccc(-c3ccc(F)cc3C(F)(F)F)cc12] |
| 249 | 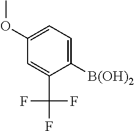 | 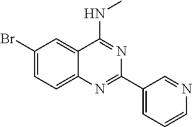 [SMILES: CNc1nc(-c2cccnc2)nc2ccc(Br)cc12] | 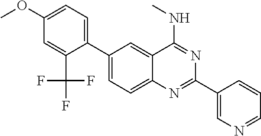 [SMILES: CNc1nc(-c2cccnc2)nc2ccc(-c3ccc(OC)cc3C(F)(F)F)cc12] |
| 250 | 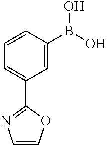 [SMILES: OB(O)c1cccc(-c2ncco2)c1] | 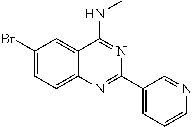 [SMILES: CNc1nc(-c2cccnc2)nc2ccc(Br)cc12] | 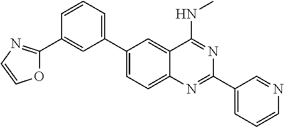 [SMILES: CNc1nc(-c2cccnc2)nc2ccc(-c3cccc(-c4ncco4)c3)cc12] |
| 251 | 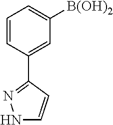 [SMILES: OB(O)c1cccc(-c2cc[nH]n2)c1] | 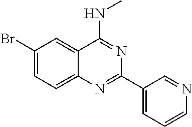 [SMILES: CNc1nc(-c2cccnc2)nc2ccc(Br)cc12] | 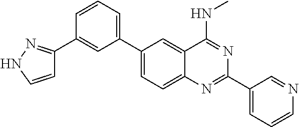 [SMILES: CNc1nc(-c2cccnc2)nc2ccc(-c3cccc(-c4cc[nH]n4)c3)cc12] |
| 252 | 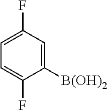 [SMILES: OB(O)c1cc(F)ccc1F] | 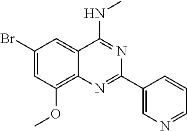 [SMILES: CNc1nc(-c2cccnc2)nc2c(OC)cc(Br)cc12] | 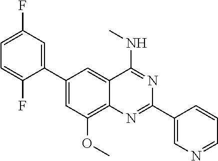 [SMILES: CNc1nc(-c2cccnc2)nc2c(OC)cc(-c3cc(F)ccc3F)cc12] |
| 253 | 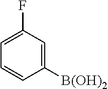 | 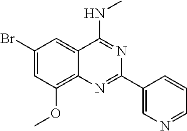 [SMILES: CNc1nc(-c2cccnc2)nc2c(OC)cc(Br)cc12] | 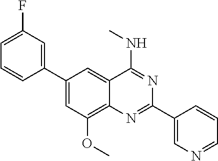 [SMILES: CNc1nc(-c2cccnc2)nc2c(OC)cc(-c3cccc(F)c3)cc12] |
| 254 | 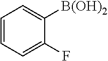 | 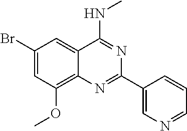 [SMILES: CNc1nc(-c2cccnc2)nc2c(OC)cc(Br)cc12] | 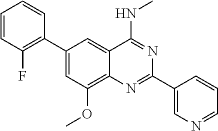 [SMILES: CNc1nc(-c2cccnc2)nc2c(OC)cc(-c3ccccc3F)cc12] |
| 255 | 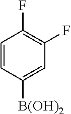 [SMILES: OB(O)c1ccc(F)c(F)c1] | 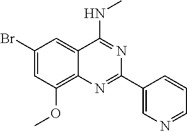 [SMILES: CNc1nc(-c2cccnc2)nc2c(OC)cc(Br)cc12] | 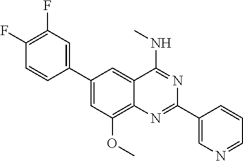 [SMILES: CNc1nc(-c2cccnc2)nc2c(OC)cc(-c3ccc(F)c(F)c3)cc12] |
| 256 | 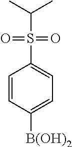 | 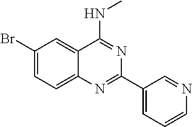 [SMILES: CNc1nc(-c2cccnc2)nc2ccc(Br)cc12] | 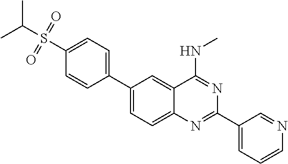 [SMILES: CNc1nc(-c2cccnc2)nc2ccc(-c3ccc(S(=O)(=O)C(C)C)cc3)cc12] |
| 257 | 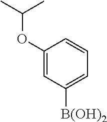 | 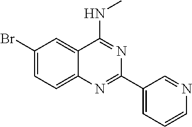 [SMILES: CNc1nc(-c2cccnc2)nc2ccc(Br)cc12] | 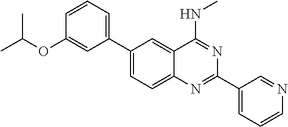 [SMILES: CNc1nc(-c2cccnc2)nc2ccc(-c3cccc(OC(C)C)c3)cc12] |
| 258 | 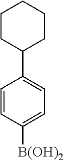 [SMILES: OB(O)c1ccc(C2CCCCC2)cc1] | 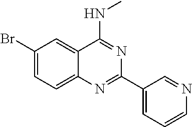 [SMILES: CNc1nc(-c2cccnc2)nc2ccc(Br)cc12] | 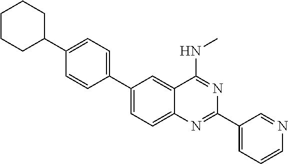 [SMILES: CNc1nc(-c2cccnc2)nc2ccc(-c3ccc(C4CCCCC4)cc3)cc12] |
| 259 | 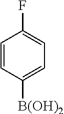 | 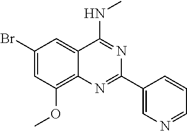 [SMILES: CNc1nc(-c2cccnc2)nc2c(OC)cc(Br)cc12] | 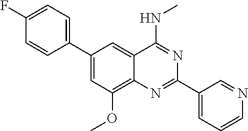 [SMILES: CNc1nc(-c2cccnc2)nc2c(OC)cc(-c3ccc(F)cc3)cc12] |
| 260 | 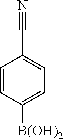 [SMILES: N#Cc1ccc(B(O)O)cc1] | 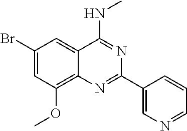 [SMILES: CNc1nc(-c2cccnc2)nc2c(OC)cc(Br)cc12] | 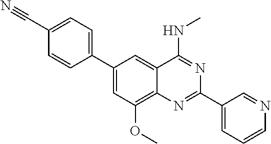 [SMILES: CNc1nc(-c2cccnc2)nc2c(OC)cc(-c3ccc(C#N)cc3)cc12] |
| 261 | 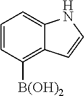 [SMILES: OB(O)c1cccc2[nH]ccc12] | 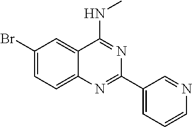 [SMILES: CNc1nc(-c2cccnc2)nc2ccc(Br)cc12] | 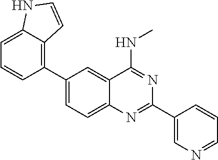 [SMILES: CNc1nc(-c2cccnc2)nc2ccc(-c3cccc4[nH]ccc34)cc12] |
| 262 | 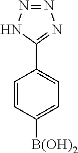 [SMILES: OB(O)c1ccc(-c2nnn[nH]2)cc1] | 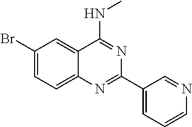 [SMILES: CNc1nc(-c2cccnc2)nc2ccc(Br)cc12] | 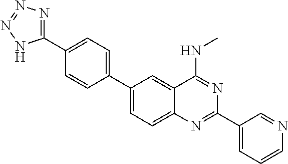 [SMILES: CNc1nc(-c2cccnc2)nc2ccc(-c3ccc(-c4nnn[nH]4)cc3)cc12] |
| 263 | 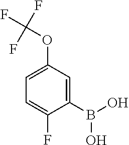 [SMILES: OB(O)c1cc(OC(F)(F)F)ccc1F] | 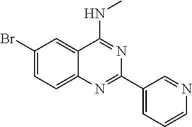 [SMILES: CNc1nc(-c2cccnc2)nc2ccc(Br)cc12] | 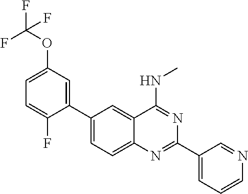 [SMILES: CNc1nc(-c2cccnc2)nc2ccc(-c3cc(OC(F)(F)F)ccc3F)cc12] |
| 264 | 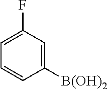 | 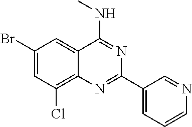 [SMILES: CNc1nc(-c2cccnc2)nc2c(Cl)cc(Br)cc12] | 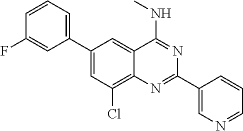 [SMILES: CNc1nc(-c2cccnc2)nc2c(Cl)cc(-c3cccc(F)c3)cc12] |
| 265 | 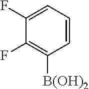 [SMILES: OB(O)c1cccc(F)c1F] | 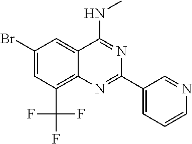 [SMILES: CNc1nc(-c2cccnc2)nc2c(C(F)(F)F)cc(Br)cc12] | 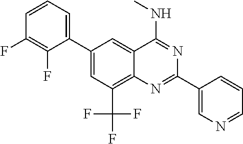 [SMILES: CNc1nc(-c2cccnc2)nc2c(C(F)(F)F)cc(-c3cccc(F)c3F)cc12] |
| 266 | 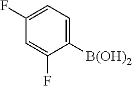 [SMILES: OB(O)c1ccc(F)cc1F] | 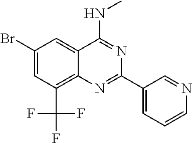 [SMILES: CNc1nc(-c2cccnc2)nc2c(C(F)(F)F)cc(Br)cc12] | 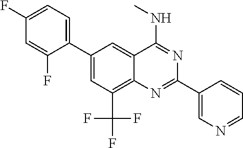 [SMILES: CNc1nc(-c2cccnc2)nc2c(C(F)(F)F)cc(-c3ccc(F)cc3F)cc12] |
| 267 | 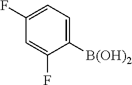 [SMILES: OB(O)c1ccc(F)cc1F] | 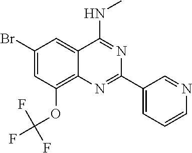 [SMILES: CNc1nc(-c2cccnc2)nc2c(OC(F)(F)F)cc(Br)cc12] | 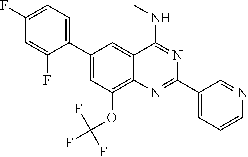 [SMILES: CNc1nc(-c2cccnc2)nc2c(OC(F)(F)F)cc(-c3ccc(F)cc3F)cc12] |
| 268 | 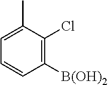 | 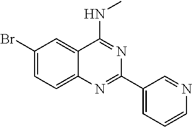 [SMILES: CNc1nc(-c2cccnc2)nc2ccc(Br)cc12] | 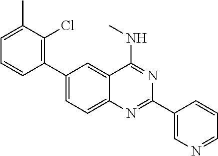 [SMILES: CNc1nc(-c2cccnc2)nc2ccc(-c3cccc(C)c3Cl)cc12] |
| 269 | 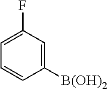 | 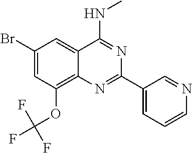 [SMILES: CNc1nc(-c2cccnc2)nc2c(OC(F)(F)F)cc(Br)cc12] | 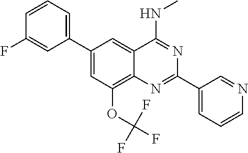 [SMILES: CNc1nc(-c2cccnc2)nc2c(OC(F)(F)F)cc(-c3cccc(F)c3)cc12] |
| 270 | 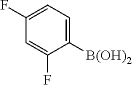 [SMILES: OB(O)c1ccc(F)cc1F] | 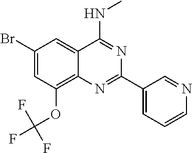 [SMILES: CNc1nc(-c2cccnc2)nc2c(OC(F)(F)F)cc(Br)cc12] | 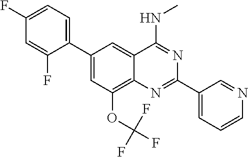 [SMILES: CNc1nc(-c2cccnc2)nc2c(OC(F)(F)F)cc(-c3ccc(F)cc3F)cc12] |
| 271 | 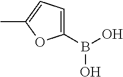 | 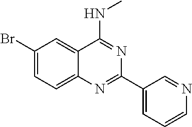 [SMILES: CNc1nc(-c2cccnc2)nc2ccc(Br)cc12] | 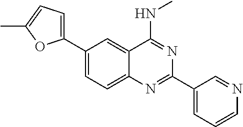 [SMILES: CNc1nc(-c2cccnc2)nc2ccc(-c3ccc(C)o3)cc12] |
| 272 | 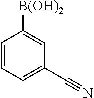 [SMILES: N#Cc1cccc(B(O)O)c1] | 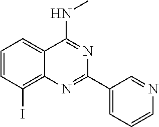 [SMILES: CNc1nc(-c2cccnc2)nc2c(I)cccc12] | 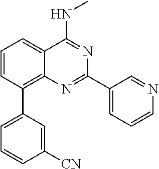 [SMILES: CNc1nc(-c2cccnc2)nc2c(-c3cccc(C#N)c3)cccc12] |
| 273 | 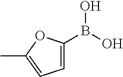 | 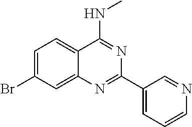 [SMILES: CNc1nc(-c2cccnc2)nc2cc(Br)ccc12] | 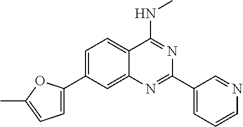 [SMILES: CNc1nc(-c2cccnc2)nc2cc(-c3ccc(C)o3)ccc12] |
| 274 | 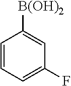 | 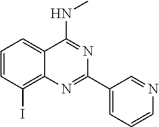 [SMILES: CNc1nc(-c2cccnc2)nc2c(I)cccc12] | 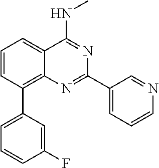 [SMILES: CNc1nc(-c2cccnc2)nc2c(-c3cccc(F)c3)cccc12] |
| 275 | 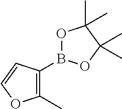 | 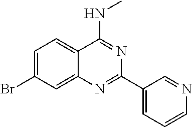 [SMILES: CNc1nc(-c2cccnc2)nc2cc(Br)ccc12] | 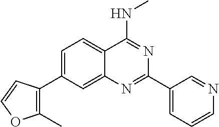 [SMILES: CNc1nc(-c2cccnc2)nc2cc(-c3ccoc3C)ccc12] |
| 276 | 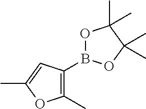 | 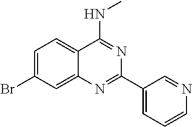 [SMILES: CNc1nc(-c2cccnc2)nc2cc(Br)ccc12] | 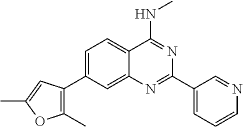 [SMILES: CNc1nc(-c2cccnc2)nc2cc(-c3cc(C)oc3C)ccc12] |
| 277 | 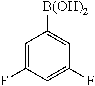 [SMILES: OB(O)c1cc(F)cc(F)c1] | 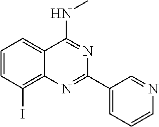 [SMILES: CNc1nc(-c2cccnc2)nc2c(I)cccc12] | 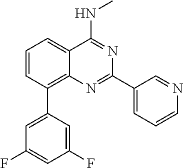 [SMILES: CNc1nc(-c2cccnc2)nc2c(-c3cc(F)cc(F)c3)cccc12] |
| 278 | 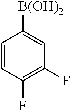 [SMILES: OB(O)c1ccc(F)c(F)c1] | 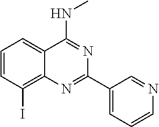 [SMILES: CNc1nc(-c2cccnc2)nc2c(I)cccc12] | 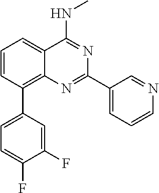 [SMILES: CNc1nc(-c2cccnc2)nc2c(-c3ccc(F)c(F)c3)cccc12] |
| 279 | 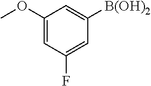 | 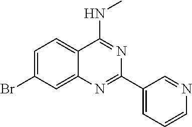 [SMILES: CNc1nc(-c2cccnc2)nc2cc(Br)ccc12] | 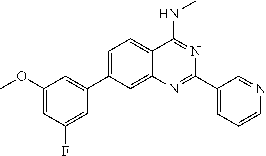 [SMILES: CNc1nc(-c2cccnc2)nc2cc(-c3cc(F)cc(OC)c3)ccc12] |
| 280 | 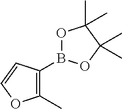 | 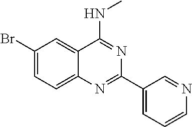 [SMILES: CNc1nc(-c2cccnc2)nc2ccc(Br)cc12] | 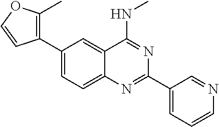 [SMILES: CNc1nc(-c2cccnc2)nc2ccc(-c3ccoc3C)cc12] |
| 281 | 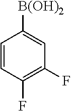 [SMILES: OB(O)c1ccc(F)c(F)c1] | 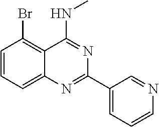 [SMILES: CNc1nc(-c2cccnc2)nc2cccc(Br)c12] | 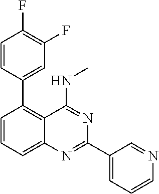 [SMILES: CNc1nc(-c2cccnc2)nc2cccc(-c3ccc(F)c(F)c3)c12] |
| 282 | 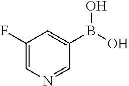 | 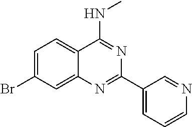 [SMILES: CNc1nc(-c2cccnc2)nc2cc(Br)ccc12] | 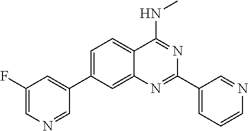 [SMILES: CNc1nc(-c2cccnc2)nc2cc(-c3cncc(F)c3)ccc12] |
| 283 | 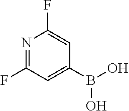 [SMILES: OB(O)c1cc(F)nc(F)c1] | 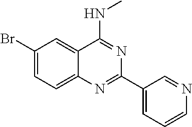 [SMILES: CNc1nc(-c2cccnc2)nc2ccc(Br)cc12] | 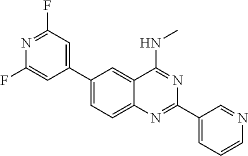 [SMILES: CNc1nc(-c2cccnc2)nc2ccc(-c3cc(F)nc(F)c3)cc12] |
| 284 | 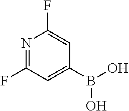 [SMILES: OB(O)c1cc(F)nc(F)c1] | 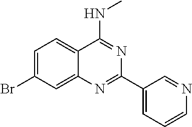 [SMILES: CNc1nc(-c2cccnc2)nc2cc(Br)ccc12] | 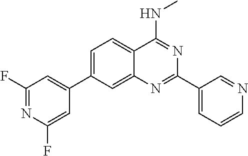 [SMILES: CNc1nc(-c2cccnc2)nc2cc(-c3cc(F)nc(F)c3)ccc12] |
| 285 | 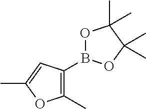 | 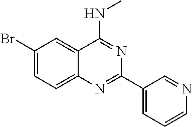 [SMILES: CNc1nc(-c2cccnc2)nc2ccc(Br)cc12] | 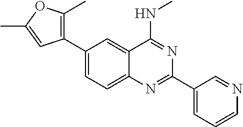 [SMILES: CNc1nc(-c2cccnc2)nc2ccc(-c3cc(C)oc3C)cc12] |
| 286 | 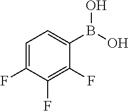 [SMILES: OB(O)c1ccc(F)c(F)c1F] | 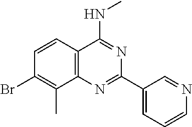 [SMILES: CNc1nc(-c2cccnc2)nc2c(C)c(Br)ccc12] | 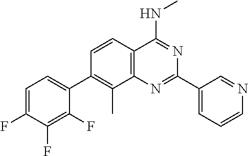 [SMILES: CNc1nc(-c2cccnc2)nc2c(C)c(-c3ccc(F)c(F)c3F)ccc12] |
| 287 | 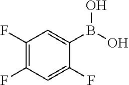 [SMILES: OB(O)c1cc(F)c(F)cc1F] | 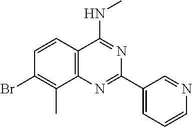 [SMILES: CNc1nc(-c2cccnc2)nc2c(C)c(Br)ccc12] | 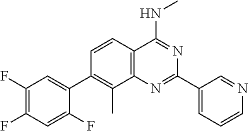 [SMILES: CNc1nc(-c2cccnc2)nc2c(C)c(-c3cc(F)c(F)cc3F)ccc12] |
| 288 | 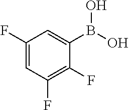 [SMILES: OB(O)c1cc(F)cc(F)c1F] | 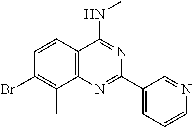 [SMILES: CNc1nc(-c2cccnc2)nc2c(C)c(Br)ccc12] | 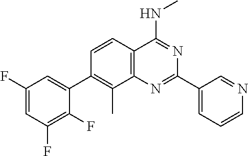 [SMILES: CNc1nc(-c2cccnc2)nc2c(C)c(-c3cc(F)cc(F)c3F)ccc12] |
| 289 | 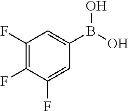 [SMILES: OB(O)c1cc(F)c(F)c(F)c1] | 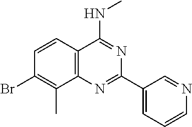 [SMILES: CNc1nc(-c2cccnc2)nc2c(C)c(Br)ccc12] | 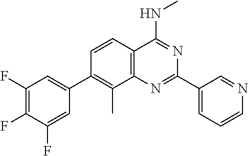 [SMILES: CNc1nc(-c2cccnc2)nc2c(C)c(-c3cc(F)c(F)c(F)c3)ccc12] |
| 290 | 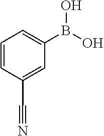 [SMILES: N#Cc1cccc(B(O)O)c1] | 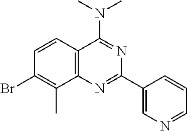 [SMILES: Cc1c(Br)ccc2c(N(C)C)nc(-c3cccnc3)nc12] | 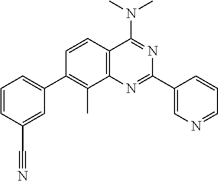 [SMILES: Cc1c(-c2cccc(C#N)c2)ccc2c(N(C)C)nc(-c3cccnc3)nc12] |
| 291 | 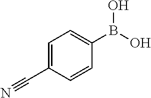 [SMILES: N#Cc1ccc(B(O)O)cc1] | 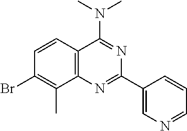 [SMILES: Cc1c(Br)ccc2c(N(C)C)nc(-c3cccnc3)nc12] | 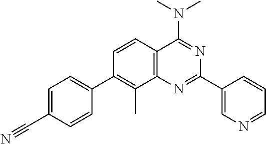 [SMILES: Cc1c(-c2ccc(C#N)cc2)ccc2c(N(C)C)nc(-c3cccnc3)nc12] |
| 292 | 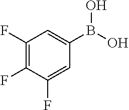 [SMILES: OB(O)c1cc(F)c(F)c(F)c1] | 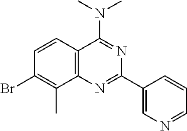 [SMILES: Cc1c(Br)ccc2c(N(C)C)nc(-c3cccnc3)nc12] | 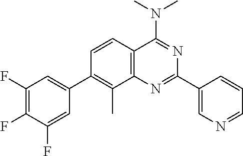 [SMILES: Cc1c(-c2cc(F)c(F)c(F)c2)ccc2c(N(C)C)nc(-c3cccnc3)nc12] |
| 293 | 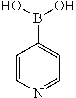 | 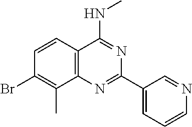 [SMILES: CNc1nc(-c2cccnc2)nc2c(C)c(Br)ccc12] | 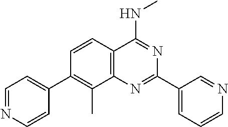 [SMILES: CNc1nc(-c2cccnc2)nc2c(C)c(-c3ccncc3)ccc12] |
| 294 | 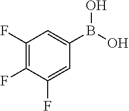 [SMILES: OB(O)c1cc(F)c(F)c(F)c1] | 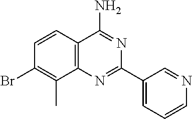 [SMILES: Cc1c(Br)ccc2c(N)nc(-c3cccnc3)nc12] | 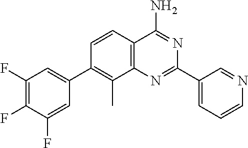 [SMILES: Cc1c(-c2cc(F)c(F)c(F)c2)ccc2c(N)nc(-c3cccnc3)nc12] |
| 295 | 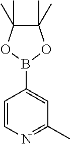 | 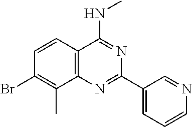 [SMILES: CNc1nc(-c2cccnc2)nc2c(C)c(Br)ccc12] | 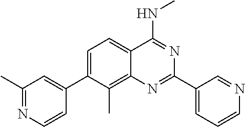 [SMILES: CNc1nc(-c2cccnc2)nc2c(C)c(-c3ccnc(C)c3)ccc12] |
| 296 | 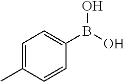 | 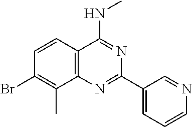 [SMILES: CNc1nc(-c2cccnc2)nc2c(C)c(Br)ccc12] | 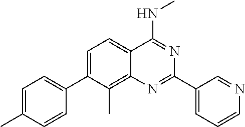 [SMILES: CNc1nc(-c2cccnc2)nc2c(C)c(-c3ccc(C)cc3)ccc12] |
| 297 | 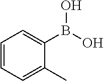 | 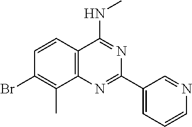 [SMILES: CNc1nc(-c2cccnc2)nc2c(C)c(Br)ccc12] | 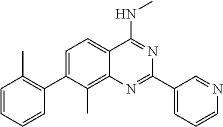 [SMILES: CNc1nc(-c2cccnc2)nc2c(C)c(-c3ccccc3C)ccc12] |
| 298 | 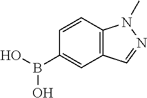 | 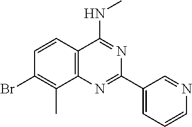 [SMILES: CNc1nc(-c2cccnc2)nc2c(C)c(Br)ccc12] | 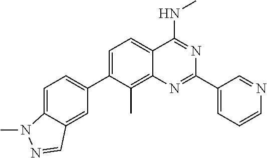 [SMILES: CNc1nc(-c2cccnc2)nc2c(C)c(-c3ccc4c(cnn4C)c3)ccc12] |
| 299 | 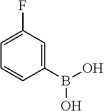 | 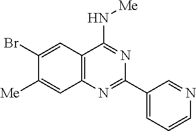 [SMILES: CNc1nc(-c2cccnc2)nc2cc(C)c(Br)cc12] | 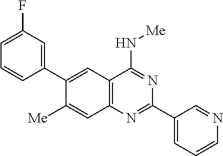 [SMILES: CNc1nc(-c2cccnc2)nc2cc(C)c(-c3cccc(F)c3)cc12] |
| 300 | 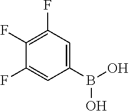 [SMILES: OB(O)c1cc(F)c(F)c(F)c1] | 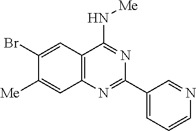 [SMILES: CNc1nc(-c2cccnc2)nc2cc(C)c(Br)cc12] | 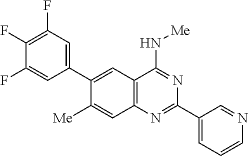 [SMILES: CNc1nc(-c2cccnc2)nc2cc(C)c(-c3cc(F)c(F)c(F)c3)cc12] |
| 301 | 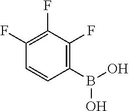 [SMILES: OB(O)c1ccc(F)c(F)c1F] | 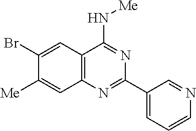 [SMILES: CNc1nc(-c2cccnc2)nc2cc(C)c(Br)cc12] | 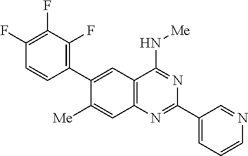 [SMILES: CNc1nc(-c2cccnc2)nc2cc(C)c(-c3ccc(F)c(F)c3F)cc12] |
| 302 | 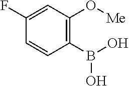 [SMILES: COc1cc(F)ccc1B(O)O] | 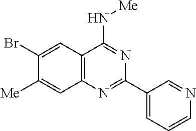 [SMILES: CNc1nc(-c2cccnc2)nc2cc(C)c(Br)cc12] | 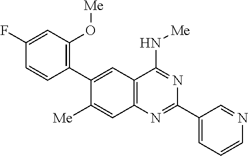 [SMILES: CNc1nc(-c2cccnc2)nc2cc(C)c(-c3ccc(F)cc3OC)cc12] |
| 303 | 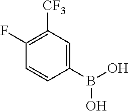 [SMILES: OB(O)c1ccc(F)c(C(F)(F)F)c1] | 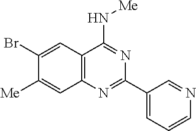 [SMILES: CNc1nc(-c2cccnc2)nc2cc(C)c(Br)cc12] | 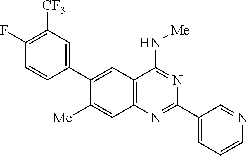 [SMILES: CNc1nc(-c2cccnc2)nc2cc(C)c(-c3ccc(F)c(C(F)(F)F)c3)cc12] |
| 304 | 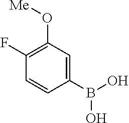 [SMILES: COc1cc(B(O)O)ccc1F] | 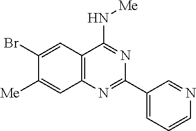 [SMILES: CNc1nc(-c2cccnc2)nc2cc(C)c(Br)cc12] | 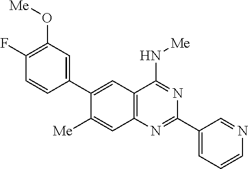 [SMILES: CNc1nc(-c2cccnc2)nc2cc(C)c(-c3ccc(F)c(OC)c3)cc12] |
| 305 | 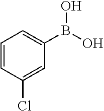 | 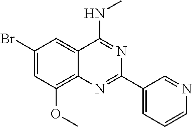 [SMILES: CNc1nc(-c2cccnc2)nc2c(OC)cc(Br)cc12] | 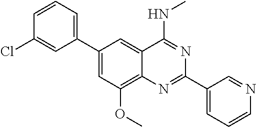 [SMILES: CNc1nc(-c2cccnc2)nc2c(OC)cc(-c3cccc(Cl)c3)cc12] |
| 306 | 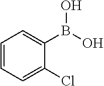 | 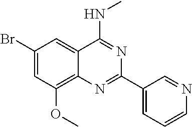 [SMILES: CNc1nc(-c2cccnc2)nc2c(OC)cc(Br)cc12] | 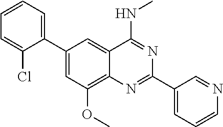 [SMILES: CNc1nc(-c2cccnc2)nc2c(OC)cc(-c3ccccc3Cl)cc12] |
| 307 | 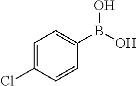 | 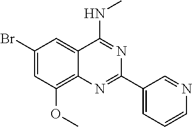 [SMILES: CNc1nc(-c2cccnc2)nc2c(OC)cc(Br)cc12] | 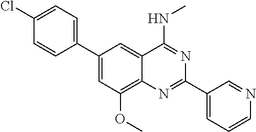 [SMILES: CNc1nc(-c2cccnc2)nc2c(OC)cc(-c3ccc(Cl)cc3)cc12] |
| 308 | 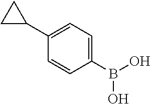 [SMILES: OB(O)c1ccc(C2CC2)cc1] | 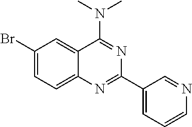 [SMILES: CN(C)c1nc(-c2cccnc2)nc2ccc(Br)cc12] | 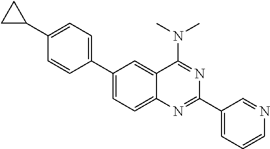 [SMILES: CN(C)c1nc(-c2cccnc2)nc2ccc(-c3ccc(C4CC4)cc3)cc12] |
| 309 | 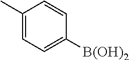 | 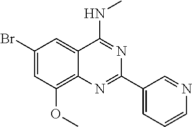 [SMILES: CNc1nc(-c2cccnc2)nc2c(OC)cc(Br)cc12] | 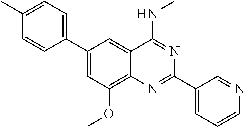 [SMILES: CNc1nc(-c2cccnc2)nc2c(OC)cc(-c3ccc(C)cc3)cc12] |
| 310 | 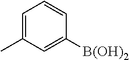 | 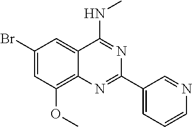 [SMILES: CNc1nc(-c2cccnc2)nc2c(OC)cc(Br)cc12] | 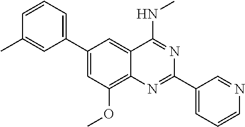 [SMILES: CNc1nc(-c2cccnc2)nc2c(OC)cc(-c3cccc(C)c3)cc12] |
| 311 | 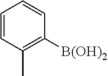 | 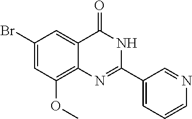 [SMILES: COc1cc(Br)cc2c(=O)[nH]c(-c3cccnc3)nc12] | 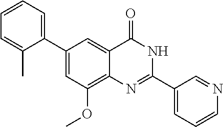 [SMILES: COc1cc(-c2ccccc2C)cc2c(=O)[nH]c(-c3cccnc3)nc12] |
| 312 | 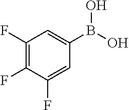 [SMILES: OB(O)c1cc(F)c(F)c(F)c1] | 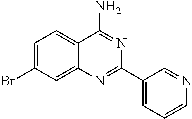 [SMILES: Nc1nc(-c2cccnc2)nc2cc(Br)ccc12] | 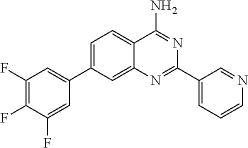 [SMILES: Nc1nc(-c2cccnc2)nc2cc(-c3cc(F)c(F)c(F)c3)ccc12] |
| 313 | 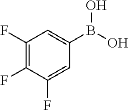 [SMILES: OB(O)c1cc(F)c(F)c(F)c1] | 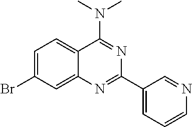 [SMILES: CN(C)c1nc(-c2cccnc2)nc2cc(Br)ccc12] | 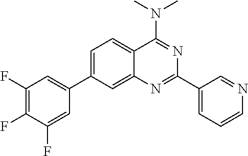 [SMILES: CN(C)c1nc(-c2cccnc2)nc2cc(-c3cc(F)c(F)c(F)c3)ccc12] |
| 314 | 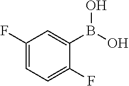 [SMILES: OB(O)c1cc(F)ccc1F] | 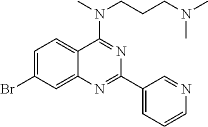 [SMILES: CN(C)CCCN(C)c1nc(-c2cccnc2)nc2cc(Br)ccc12] | 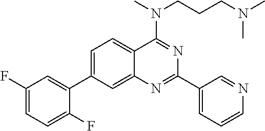 [SMILES: CN(C)CCCN(C)c1nc(-c2cccnc2)nc2cc(-c3cc(F)ccc3F)ccc12] |
| 315 | 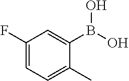 | 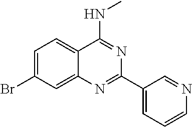 [SMILES: CNc1nc(-c2cccnc2)nc2cc(Br)ccc12] | 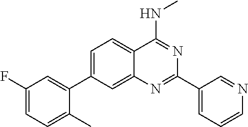 [SMILES: CNc1nc(-c2cccnc2)nc2cc(-c3cc(F)ccc3C)ccc12] |
| 316 | 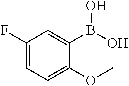 | 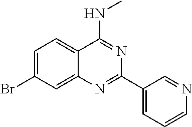 [SMILES: CNc1nc(-c2cccnc2)nc2cc(Br)ccc12] | 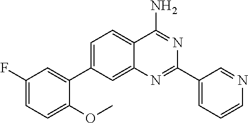 [SMILES: COc1ccc(F)cc1-c1ccc2c(N)nc(-c3cccnc3)nc2c1] |
| 317 | 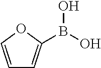 | 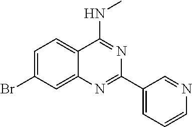 [SMILES: CNc1nc(-c2cccnc2)nc2cc(Br)ccc12] | 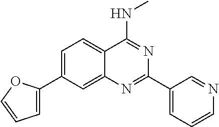 [SMILES: CNc1nc(-c2cccnc2)nc2cc(-c3ccco3)ccc12] |
| 318 | 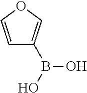 | 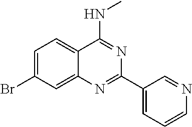 [SMILES: CNc1nc(-c2cccnc2)nc2cc(Br)ccc12] | 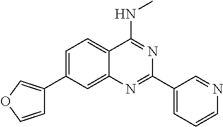 [SMILES: CNc1nc(-c2cccnc2)nc2cc(-c3ccoc3)ccc12] |
| 319 | 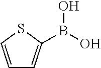 | 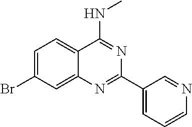 [SMILES: CNc1nc(-c2cccnc2)nc2cc(Br)ccc12] | 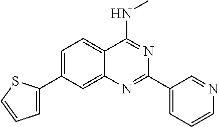 [SMILES: CNc1nc(-c2cccnc2)nc2cc(-c3cccs3)ccc12] |
| 320 | 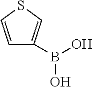 | 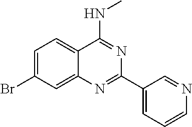 [SMILES: CNc1nc(-c2cccnc2)nc2cc(Br)ccc12] | 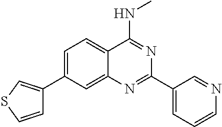 [SMILES: CNc1nc(-c2cccnc2)nc2cc(-c3ccsc3)ccc12] |
| 321 | 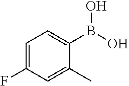 | 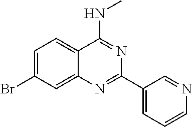 [SMILES: CNc1nc(-c2cccnc2)nc2cc(Br)ccc12] | 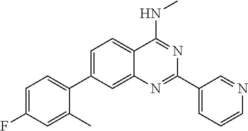 [SMILES: CNc1nc(-c2cccnc2)nc2cc(-c3ccc(F)cc3C)ccc12] |
| 322 | 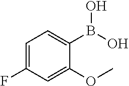 | 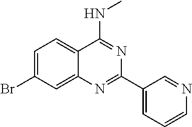 [SMILES: CNc1nc(-c2cccnc2)nc2cc(Br)ccc12] | 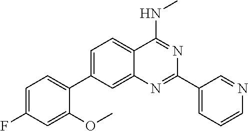 [SMILES: CNc1nc(-c2cccnc2)nc2cc(-c3ccc(F)cc3OC)ccc12] |
| 323 | 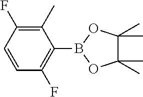 [SMILES: Cc1c(F)ccc(F)c1B1OC(C)(C)C(C)(C)O1] | 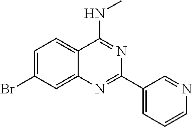 [SMILES: CNc1nc(-c2cccnc2)nc2cc(Br)ccc12] | 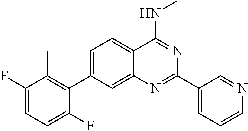 [SMILES: CNc1nc(-c2cccnc2)nc2cc(-c3c(F)ccc(F)c3C)ccc12] |
| 324 | 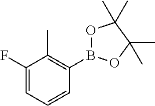 [SMILES: Cc1c(F)cccc1B1OC(C)(C)C(C)(C)O1] | 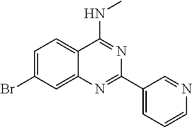 [SMILES: CNc1nc(-c2cccnc2)nc2cc(Br)ccc12] | 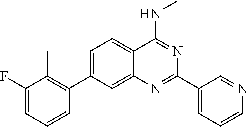 [SMILES: CNc1nc(-c2cccnc2)nc2cc(-c3cccc(F)c3C)ccc12] |
| 325 | 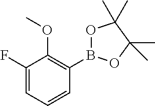 [SMILES: COc1c(F)cccc1B1OC(C)(C)C(C)(C)O1] | 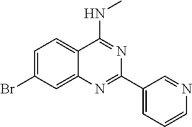 [SMILES: CNc1nc(-c2cccnc2)nc2cc(Br)ccc12] | 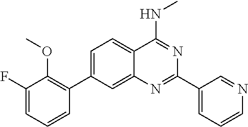 [SMILES: CNc1nc(-c2cccnc2)nc2cc(-c3cccc(F)c3OC)ccc12] |
| 326 | 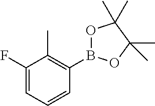 [SMILES: Cc1c(F)cccc1B1OC(C)(C)C(C)(C)O1] | 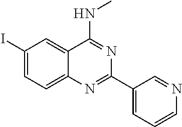 [SMILES: CNc1nc(-c2cccnc2)nc2ccc(I)cc12] | 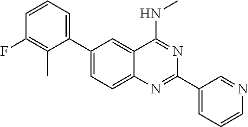 [SMILES: CNc1nc(-c2cccnc2)nc2ccc(-c3cccc(F)c3C)cc12] |
| 327 | 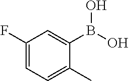 | 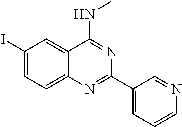 [SMILES: CNc1nc(-c2cccnc2)nc2ccc(I)cc12] | 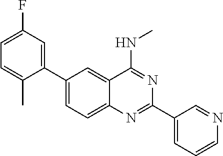 [SMILES: CNc1nc(-c2cccnc2)nc2ccc(-c3cc(F)ccc3C)cc12] |
| 328 | 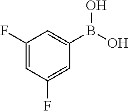 [SMILES: OB(O)c1cc(F)cc(F)c1] | 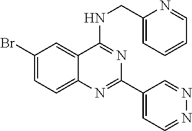 [SMILES: Brc1ccc2nc(-c3ccnnc3)nc(NCc3ccccn3)c2c1] | 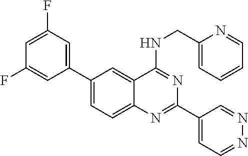 [SMILES: Fc1cc(F)cc(-c2ccc3nc(-c4ccnnc4)nc(NCc4ccccn4)c3c2)c1] |
| 329 | 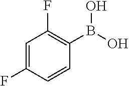 [SMILES: OB(O)c1ccc(F)cc1F] | 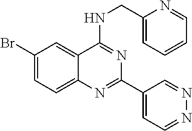 [SMILES: Brc1ccc2nc(-c3ccnnc3)nc(NCc3ccccn3)c2c1] | 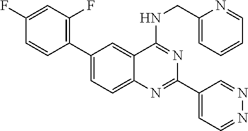 [SMILES: Fc1ccc(-c2ccc3nc(-c4ccnnc4)nc(NCc4ccccn4)c3c2)c(F)c1] |
| 330 | 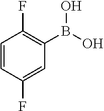 [SMILES: OB(O)c1cc(F)ccc1F] | 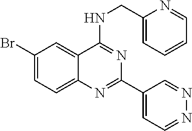 [SMILES: Brc1ccc2nc(-c3ccnnc3)nc(NCc3ccccn3)c2c1] | 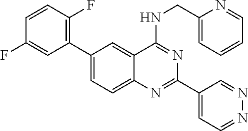 [SMILES: Fc1ccc(F)c(-c2ccc3nc(-c4ccnnc4)nc(NCc4ccccn4)c3c2)c1] |
| 331 | 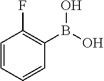 | 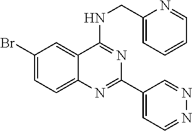 [SMILES: Brc1ccc2nc(-c3ccnnc3)nc(NCc3ccccn3)c2c1] | 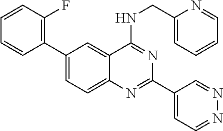 [SMILES: Fc1ccccc1-c1ccc2nc(-c3ccnnc3)nc(NCc3ccccn3)c2c1] |
| 332 | 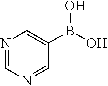 | 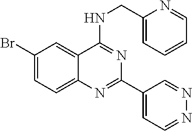 [SMILES: Brc1ccc2nc(-c3ccnnc3)nc(NCc3ccccn3)c2c1] | 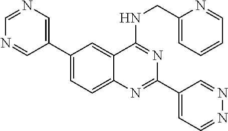 [SMILES: c1ccc(CNc2nc(-c3ccnnc3)nc3ccc(-c4cncnc4)cc23)nc1] |
| 333 | 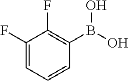 [SMILES: OB(O)c1cccc(F)c1F] | 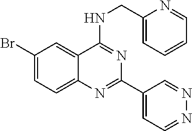 [SMILES: Brc1ccc2nc(-c3ccnnc3)nc(NCc3ccccn3)c2c1] | 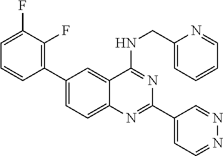 [SMILES: Fc1cccc(-c2ccc3nc(-c4ccnnc4)nc(NCc4ccccn4)c3c2)c1F] |
| 334 | 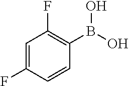 [SMILES: OB(O)c1ccc(F)cc1F] | 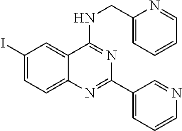 [SMILES: Ic1ccc2nc(-c3cccnc3)nc(NCc3ccccn3)c2c1] | 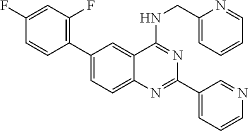 [SMILES: Fc1ccc(-c2ccc3nc(-c4cccnc4)nc(NCc4ccccn4)c3c2)c(F)c1] |
| 335 | 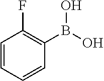 | 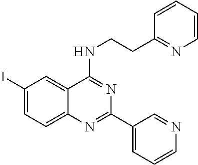 [SMILES: Ic1ccc2nc(-c3cccnc3)nc(NCCc3ccccn3)c2c1] | 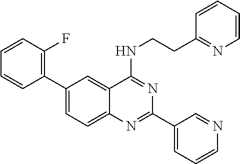 [SMILES: Fc1ccccc1-c1ccc2nc(-c3cccnc3)nc(NCCc3ccccn3)c2c1] |
| 336 | 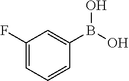 | 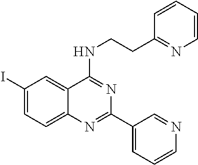 [SMILES: Ic1ccc2nc(-c3cccnc3)nc(NCCc3ccccn3)c2c1] | 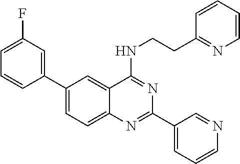 [SMILES: Fc1cccc(-c2ccc3nc(-c4cccnc4)nc(NCCc4ccccn4)c3c2)c1] |
| 337 | 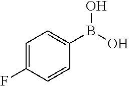 | 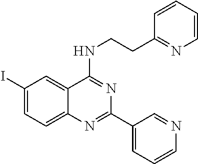 [SMILES: Ic1ccc2nc(-c3cccnc3)nc(NCCc3ccccn3)c2c1] | 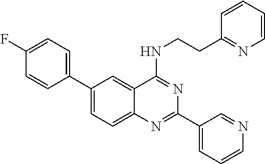 [SMILES: Fc1ccc(-c2ccc3nc(-c4cccnc4)nc(NCCc4ccccn4)c3c2)cc1] |
| 338 | 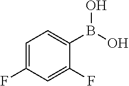 [SMILES: OB(O)c1ccc(F)cc1F] | 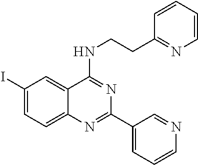 [SMILES: Ic1ccc2nc(-c3cccnc3)nc(NCCc3ccccn3)c2c1] | 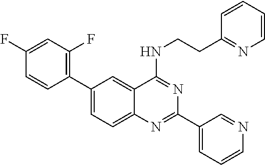 [SMILES: Fc1ccc(-c2ccc3nc(-c4cccnc4)nc(NCCc4ccccn4)c3c2)c(F)c1] |
| 339 | 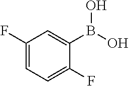 [SMILES: OB(O)c1cc(F)ccc1F] | 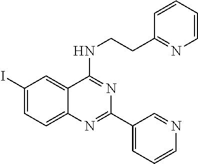 [SMILES: Ic1ccc2nc(-c3cccnc3)nc(NCCc3ccccn3)c2c1] | 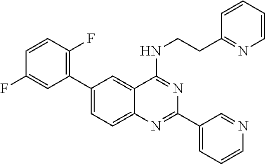 [SMILES: Fc1ccc(F)c(-c2ccc3nc(-c4cccnc4)nc(NCCc4ccccn4)c3c2)c1] |
| 340 | 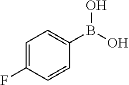 | 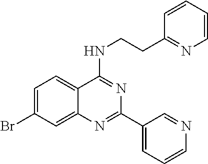 [SMILES: Brc1ccc2c(NCCc3ccccn3)nc(-c3cccnc3)nc2c1] | 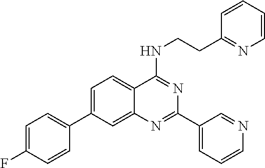 [SMILES: Fc1ccc(-c2ccc3c(NCCc4ccccn4)nc(-c4cccnc4)nc3c2)cc1] |
| 341 | 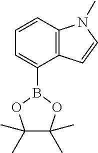 [SMILES: Cn1ccc2c(B3OC(C)(C)C(C)(C)O3)cccc21] | 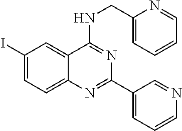 [SMILES: Ic1ccc2nc(-c3cccnc3)nc(NCc3ccccn3)c2c1] | 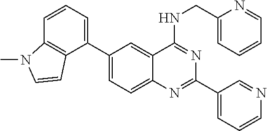 [SMILES: Cn1ccc2c(-c3ccc4nc(-c5cccnc5)nc(NCc5ccccn5)c4c3)cccc21] |
| 342 | 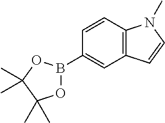 [SMILES: Cn1ccc2cc(B3OC(C)(C)C(C)(C)O3)ccc21] | 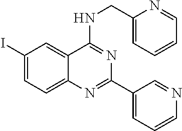 [SMILES: Ic1ccc2nc(-c3cccnc3)nc(NCc3ccccn3)c2c1] | 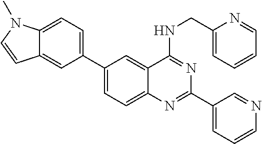 [SMILES: Cn1ccc2cc(-c3ccc4nc(-c5cccnc5)nc(NCc5ccccn5)c4c3)ccc21] |
| 343 | 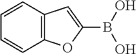 [SMILES: OB(O)c1cc2ccccc2o1] | 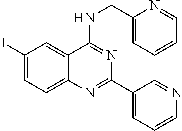 [SMILES: Ic1ccc2nc(-c3cccnc3)nc(NCc3ccccn3)c2c1] | 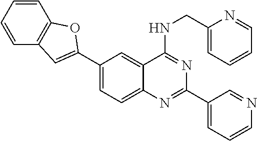 [SMILES: c1ccc(CNc2nc(-c3cccnc3)nc3ccc(-c4cc5ccccc5o4)cc23)nc1] |
| 344 | 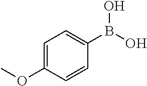 | 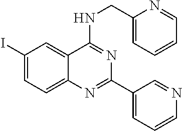 [SMILES: Ic1ccc2nc(-c3cccnc3)nc(NCc3ccccn3)c2c1] | 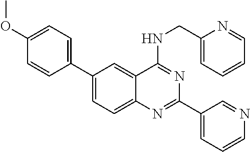 [SMILES: COc1ccc(-c2ccc3nc(-c4cccnc4)nc(NCc4ccccn4)c3c2)cc1] |
| 345 | 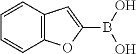 [SMILES: OB(O)c1cc2ccccc2o1] | 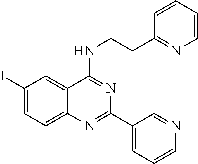 [SMILES: Ic1ccc2nc(-c3cccnc3)nc(NCCc3ccccn3)c2c1] | 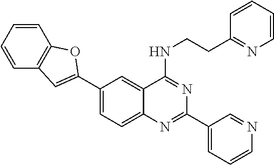 [SMILES: c1ccc(CCNc2nc(-c3cccnc3)nc3ccc(-c4cc5ccccc5o4)cc23)nc1] |
| 346 | 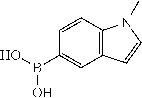 | 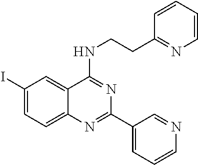 [SMILES: Ic1ccc2nc(-c3cccnc3)nc(NCCc3ccccn3)c2c1] | 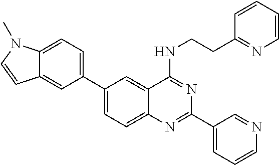 [SMILES: Cn1ccc2cc(-c3ccc4nc(-c5cccnc5)nc(NCCc5ccccn5)c4c3)ccc21] |
| 347 | 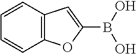 [SMILES: OB(O)c1cc2ccccc2o1] | 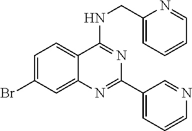 [SMILES: Brc1ccc2c(NCc3ccccn3)nc(-c3cccnc3)nc2c1] | 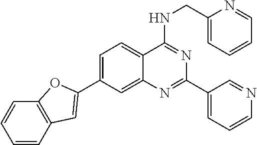 [SMILES: c1ccc(CNc2nc(-c3cccnc3)nc3cc(-c4cc5ccccc5o4)ccc23)nc1] |
| 348 | 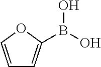 | 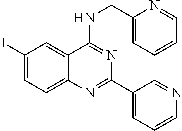 [SMILES: Ic1ccc2nc(-c3cccnc3)nc(NCc3ccccn3)c2c1] | 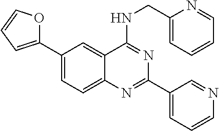 [SMILES: c1ccc(CNc2nc(-c3cccnc3)nc3ccc(-c4ccco4)cc23)nc1] |
| 601 | 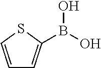 | 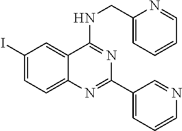 [SMILES: Ic1ccc2nc(-c3cccnc3)nc(NCc3ccccn3)c2c1] | 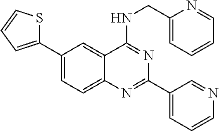 [SMILES: c1ccc(CNc2nc(-c3cccnc3)nc3ccc(-c4cccs4)cc23)nc1] |
| 602 | 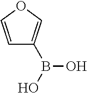 | 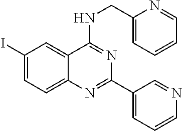 [SMILES: Ic1ccc2nc(-c3cccnc3)nc(NCc3ccccn3)c2c1] | 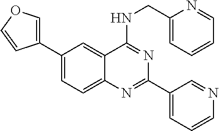 [SMILES: c1ccc(CNc2nc(-c3cccnc3)nc3ccc(-c4ccoc4)cc23)nc1] |
| 603 | 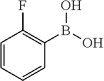 | 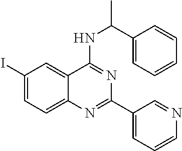 [SMILES: CC(Nc1nc(-c2cccnc2)nc2ccc(I)cc12)c1ccccc1] | 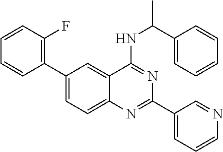 [SMILES: CC(Nc1nc(-c2cccnc2)nc2ccc(-c3ccccc3F)cc12)c1ccccc1] |
| 604 | 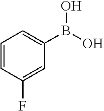 | 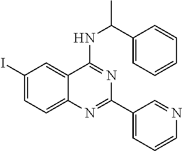 [SMILES: CC(Nc1nc(-c2cccnc2)nc2ccc(I)cc12)c1ccccc1] | 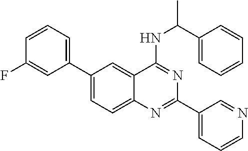 [SMILES: CC(Nc1nc(-c2cccnc2)nc2ccc(-c3cccc(F)c3)cc12)c1ccccc1] |
| 605 | 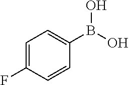 | 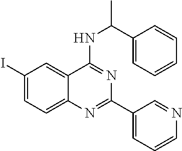 [SMILES: CC(Nc1nc(-c2cccnc2)nc2ccc(I)cc12)c1ccccc1] | 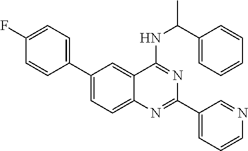 [SMILES: CC(Nc1nc(-c2cccnc2)nc2ccc(-c3ccc(F)cc3)cc12)c1ccccc1] |
| 606 | 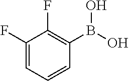 [SMILES: OB(O)c1cccc(F)c1F] | 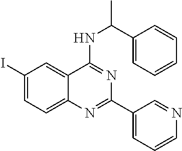 [SMILES: CC(Nc1nc(-c2cccnc2)nc2ccc(I)cc12)c1ccccc1] | 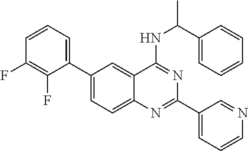 [SMILES: CC(Nc1nc(-c2cccnc2)nc2ccc(-c3cccc(F)c3F)cc12)c1ccccc1] |
| 607 | 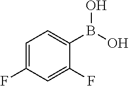 [SMILES: OB(O)c1ccc(F)cc1F] | 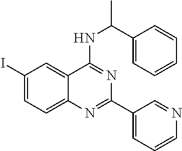 [SMILES: CC(Nc1nc(-c2cccnc2)nc2ccc(I)cc12)c1ccccc1] | 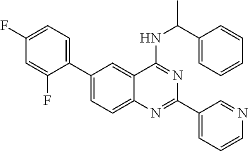 [SMILES: CC(Nc1nc(-c2cccnc2)nc2ccc(-c3ccc(F)cc3F)cc12)c1ccccc1] |
| 608 | 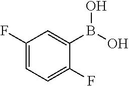 [SMILES: OB(O)c1cc(F)ccc1F] | 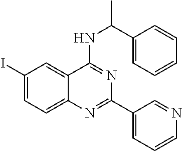 [SMILES: CC(Nc1nc(-c2cccnc2)nc2ccc(I)cc12)c1ccccc1] | 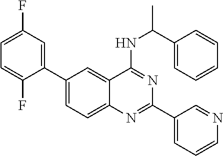 [SMILES: CC(Nc1nc(-c2cccnc2)nc2ccc(-c3cc(F)ccc3F)cc12)c1ccccc1] |
| 349 | 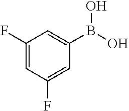 [SMILES: OB(O)c1cc(F)cc(F)c1] | 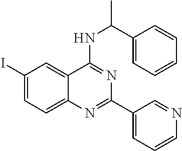 [SMILES: CC(Nc1nc(-c2cccnc2)nc2ccc(I)cc12)c1ccccc1] | 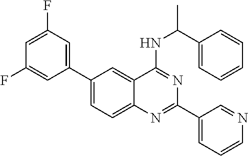 [SMILES: CC(Nc1nc(-c2cccnc2)nc2ccc(-c3cc(F)cc(F)c3)cc12)c1ccccc1] |
| 350 | 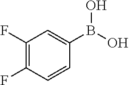 [SMILES: OB(O)c1ccc(F)c(F)c1] | 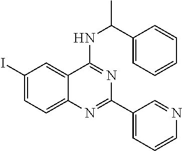 [SMILES: CC(Nc1nc(-c2cccnc2)nc2ccc(I)cc12)c1ccccc1] | 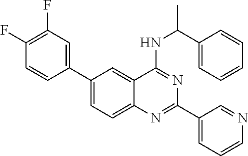 [SMILES: CC(Nc1nc(-c2cccnc2)nc2ccc(-c3ccc(F)c(F)c3)cc12)c1ccccc1] |
| 351 | 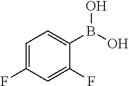 [SMILES: OB(O)c1ccc(F)cc1F] | 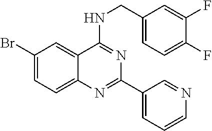 [SMILES: Fc1ccc(CNc2nc(-c3cccnc3)nc3ccc(Br)cc23)cc1F] | 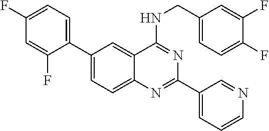 [SMILES: Fc1ccc(-c2ccc3nc(-c4cccnc4)nc(NCc4ccc(F)c(F)c4)c3c2)c(F)c1] |
| 352 | 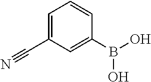 [SMILES: N#Cc1cccc(B(O)O)c1] | 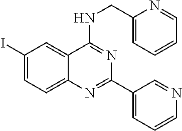 [SMILES: Ic1ccc2nc(-c3cccnc3)nc(NCc3ccccn3)c2c1] | 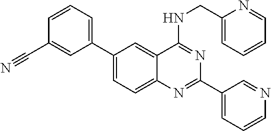 [SMILES: N#Cc1cccc(-c2ccc3nc(-c4cccnc4)nc(NCc4ccccn4)c3c2)c1] |
| 353 | 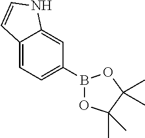 [SMILES: CC1(C)OB(c2ccc3cc[nH]c3c2)OC1(C)C] | 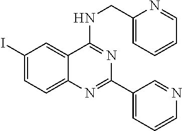 [SMILES: Ic1ccc2nc(-c3cccnc3)nc(NCc3ccccn3)c2c1] | 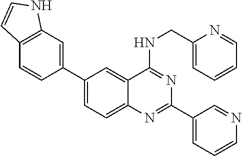 [SMILES: c1ccc(CNc2nc(-c3cccnc3)nc3ccc(-c4ccc5cc[nH]c5c4)cc23)nc1] |
| 354 | 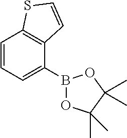 [SMILES: CC1(C)OB(c2cccc3sccc23)OC1(C)C] | 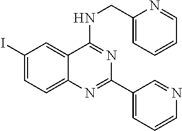 [SMILES: Ic1ccc2nc(-c3cccnc3)nc(NCc3ccccn3)c2c1] | 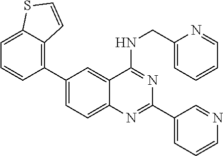 [SMILES: c1ccc(CNc2nc(-c3cccnc3)nc3ccc(-c4cccc5sccc45)cc23)nc1] |
| 355 | 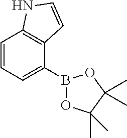 [SMILES: CC1(C)OB(c2cccc3[nH]ccc23)OC1(C)C] | 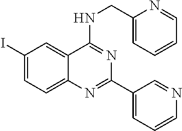 [SMILES: Ic1ccc2nc(-c3cccnc3)nc(NCc3ccccn3)c2c1] | 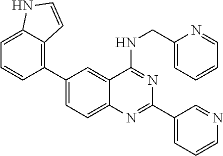 [SMILES: c1ccc(CNc2nc(-c3cccnc3)nc3ccc(-c4cccc5[nH]ccc45)cc23)nc1] |
| 356 | 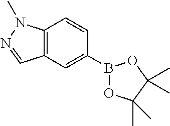 [SMILES: Cn1ncc2cc(B3OC(C)(C)C(C)(C)O3)ccc21] | 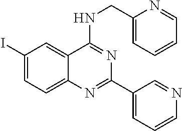 [SMILES: Ic1ccc2nc(-c3cccnc3)nc(NCc3ccccn3)c2c1] | 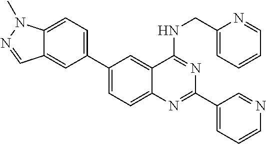 [SMILES: Cn1ncc2cc(-c3ccc4nc(-c5cccnc5)nc(NCc5ccccn5)c4c3)ccc21] |
| 357 | 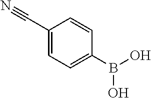 [SMILES: N#Cc1ccc(B(O)O)cc1] | 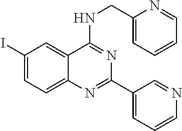 [SMILES: Ic1ccc2nc(-c3cccnc3)nc(NCc3ccccn3)c2c1] | 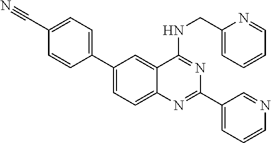 [SMILES: N#Cc1ccc(-c2ccc3nc(-c4cccnc4)nc(NCc4ccccn4)c3c2)cc1] |
| 358 | 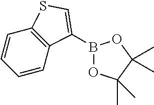 [SMILES: CC1(C)OB(c2csc3ccccc23)OC1(C)C] | 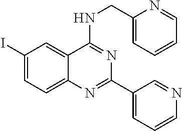 [SMILES: Ic1ccc2nc(-c3cccnc3)nc(NCc3ccccn3)c2c1] | 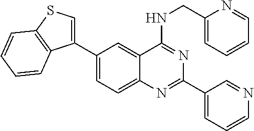 [SMILES: c1ccc(CNc2nc(-c3cccnc3)nc3ccc(-c4csc5ccccc45)cc23)nc1] |
| 359 | 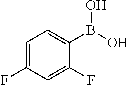 [SMILES: OB(O)c1ccc(F)cc1F] | 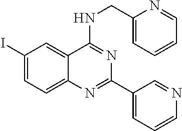 [SMILES: Ic1ccc2nc(-c3cccnc3)nc(NCc3ccccn3)c2c1] | 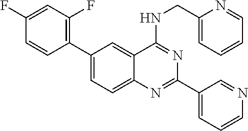 [SMILES: Fc1ccc(-c2ccc3nc(-c4cccnc4)nc(NCc4ccccn4)c3c2)c(F)c1] |
| 360 | 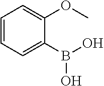 | 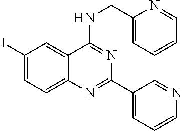 [SMILES: Ic1ccc2nc(-c3cccnc3)nc(NCc3ccccn3)c2c1] | 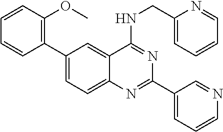 [SMILES: COc1ccccc1-c1ccc2nc(-c3cccnc3)nc(NCc3ccccn3)c2c1] |
| 361 | 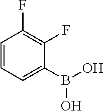 [SMILES: OB(O)c1cccc(F)c1F] | 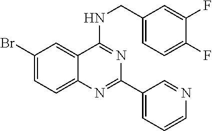 [SMILES: Fc1ccc(CNc2nc(-c3cccnc3)nc3ccc(Br)cc23)cc1F] | 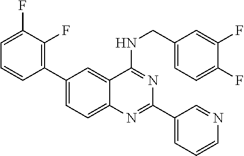 [SMILES: Fc1ccc(CNc2nc(-c3cccnc3)nc3ccc(-c4cccc(F)c4F)cc23)cc1F] |
| 362 | 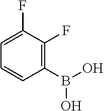 [SMILES: OB(O)c1cccc(F)c1F] | 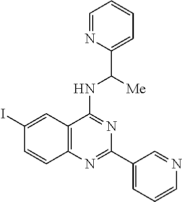 [SMILES: CC(Nc1nc(-c2cccnc2)nc2ccc(I)cc12)c1ccccn1] | 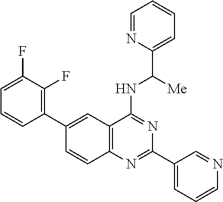 [SMILES: CC(Nc1nc(-c2cccnc2)nc2ccc(-c3cccc(F)c3F)cc12)c1ccccn1] |
| 363 | 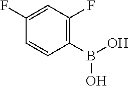 [SMILES: OB(O)c1ccc(F)cc1F] | 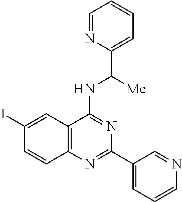 [SMILES: CC(Nc1nc(-c2cccnc2)nc2ccc(I)cc12)c1ccccn1] | 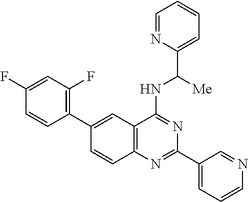 [SMILES: CC(Nc1nc(-c2cccnc2)nc2ccc(-c3ccc(F)cc3F)cc12)c1ccccn1] |
| 364 | 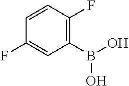 [SMILES: OB(O)c1cc(F)ccc1F] | 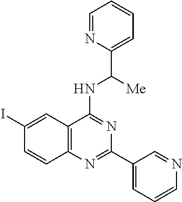 [SMILES: CC(Nc1nc(-c2cccnc2)nc2ccc(I)cc12)c1ccccn1] | 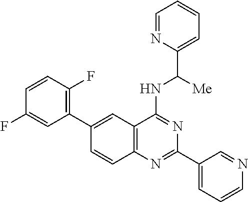 [SMILES: CC(Nc1nc(-c2cccnc2)nc2ccc(-c3cc(F)ccc3F)cc12)c1ccccn1] |
| 365 | 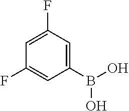 [SMILES: OB(O)c1cc(F)cc(F)c1] | 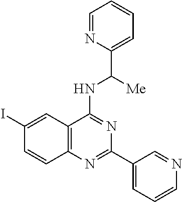 [SMILES: CC(Nc1nc(-c2cccnc2)nc2ccc(I)cc12)c1ccccn1] | 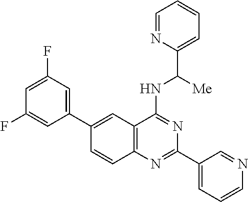 [SMILES: CC(Nc1nc(-c2cccnc2)nc2ccc(-c3cc(F)cc(F)c3)cc12)c1ccccn1] |
| 366 | 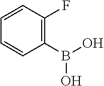 | 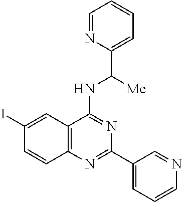 [SMILES: CC(Nc1nc(-c2cccnc2)nc2ccc(I)cc12)c1ccccn1] | 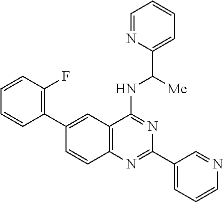 [SMILES: CC(Nc1nc(-c2cccnc2)nc2ccc(-c3ccccc3F)cc12)c1ccccn1] |
| 367 | 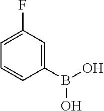 | 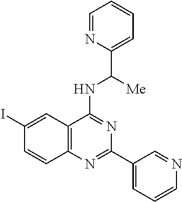 [SMILES: CC(Nc1nc(-c2cccnc2)nc2ccc(I)cc12)c1ccccn1] | 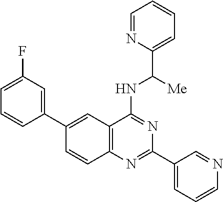 [SMILES: CC(Nc1nc(-c2cccnc2)nc2ccc(-c3cccc(F)c3)cc12)c1ccccn1] |
| 368 | 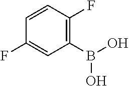 [SMILES: OB(O)c1cc(F)ccc1F] | 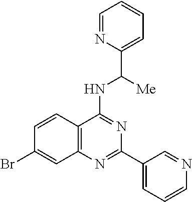 [SMILES: CC(Nc1nc(-c2cccnc2)nc2cc(Br)ccc12)c1ccccn1] | 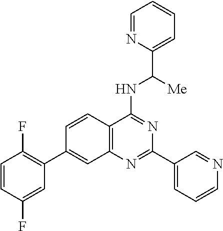 [SMILES: CC(Nc1nc(-c2cccnc2)nc2cc(-c3cc(F)ccc3F)ccc12)c1ccccn1] |
| 369 | 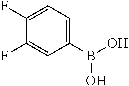 [SMILES: OB(O)c1ccc(F)c(F)c1] | 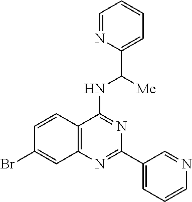 [SMILES: CC(Nc1nc(-c2cccnc2)nc2cc(Br)ccc12)c1ccccn1] | 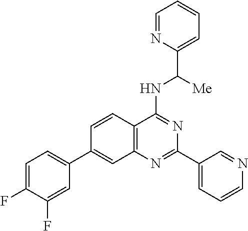 [SMILES: CC(Nc1nc(-c2cccnc2)nc2cc(-c3ccc(F)c(F)c3)ccc12)c1ccccn1] |
| 370 | 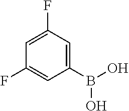 [SMILES: OB(O)c1cc(F)cc(F)c1] | 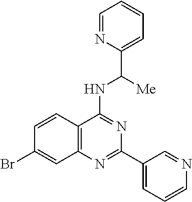 [SMILES: CC(Nc1nc(-c2cccnc2)nc2cc(Br)ccc12)c1ccccn1] | 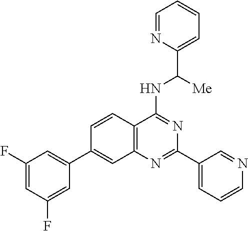 [SMILES: CC(Nc1nc(-c2cccnc2)nc2cc(-c3cc(F)cc(F)c3)ccc12)c1ccccn1] |
| 371 | 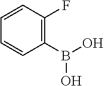 | 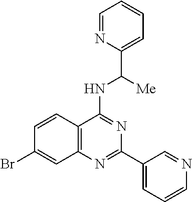 [SMILES: CC(Nc1nc(-c2cccnc2)nc2cc(Br)ccc12)c1ccccn1] | 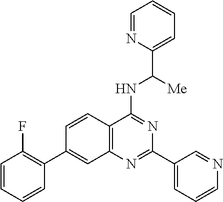 [SMILES: CC(Nc1nc(-c2cccnc2)nc2cc(-c3ccccc3F)ccc12)c1ccccn1] |
| 372 | 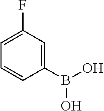 | 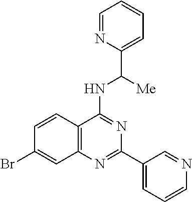 [SMILES: CC(Nc1nc(-c2cccnc2)nc2cc(Br)ccc12)c1ccccn1] | 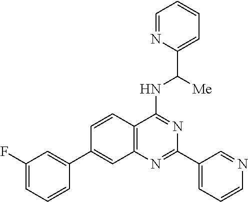 [SMILES: CC(Nc1nc(-c2cccnc2)nc2cc(-c3cccc(F)c3)ccc12)c1ccccn1] |
| 373 | 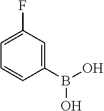 | 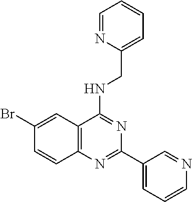 [SMILES: Brc1ccc2nc(-c3cccnc3)nc(NCc3ccccn3)c2c1] | 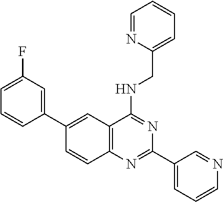 [SMILES: Fc1cccc(-c2ccc3nc(-c4cccnc4)nc(NCc4ccccn4)c3c2)c1] |
| 374 | 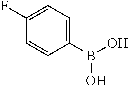 | 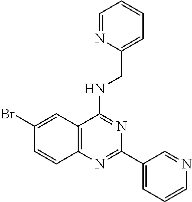 [SMILES: Brc1ccc2nc(-c3cccnc3)nc(NCc3ccccn3)c2c1] | 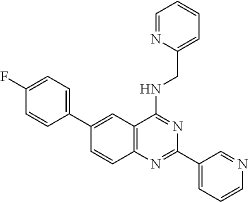 [SMILES: Fc1ccc(-c2ccc3nc(-c4cccnc4)nc(NCc4ccccn4)c3c2)cc1] |
| 375 | 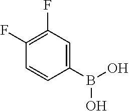 [SMILES: OB(O)c1ccc(F)c(F)c1] | 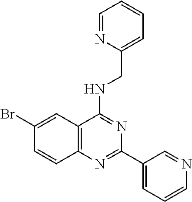 [SMILES: Brc1ccc2nc(-c3cccnc3)nc(NCc3ccccn3)c2c1] | 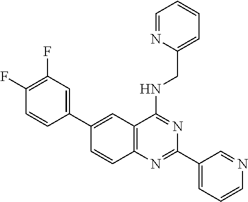 [SMILES: Fc1ccc(-c2ccc3nc(-c4cccnc4)nc(NCc4ccccn4)c3c2)cc1F] |
| 376 | 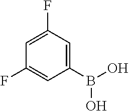 [SMILES: OB(O)c1cc(F)cc(F)c1] | 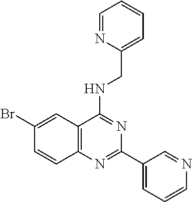 [SMILES: Brc1ccc2nc(-c3cccnc3)nc(NCc3ccccn3)c2c1] | 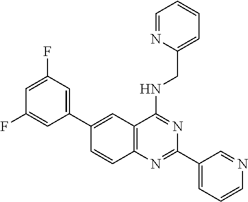 [SMILES: Fc1cc(F)cc(-c2ccc3nc(-c4cccnc4)nc(NCc4ccccn4)c3c2)c1] |
| 377 | 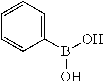 | 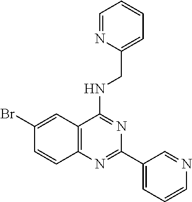 [SMILES: Brc1ccc2nc(-c3cccnc3)nc(NCc3ccccn3)c2c1] | 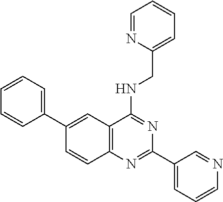 [SMILES: c1ccc(-c2ccc3nc(-c4cccnc4)nc(NCc4ccccn4)c3c2)cc1] |
| 378 | 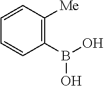 | 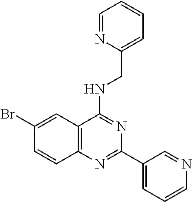 [SMILES: Brc1ccc2nc(-c3cccnc3)nc(NCc3ccccn3)c2c1] | 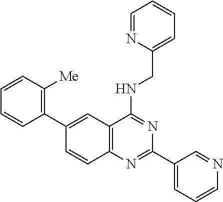 [SMILES: Cc1ccccc1-c1ccc2nc(-c3cccnc3)nc(NCc3ccccn3)c2c1] |
| 379 | 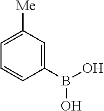 | 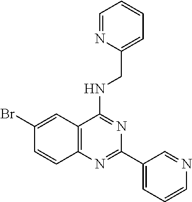 [SMILES: Brc1ccc2nc(-c3cccnc3)nc(NCc3ccccn3)c2c1] | 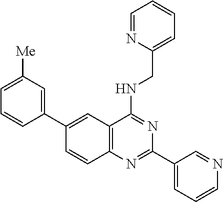 [SMILES: Cc1cccc(-c2ccc3nc(-c4cccnc4)nc(NCc4ccccn4)c3c2)c1] |
| 380 | 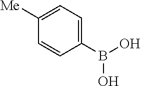 | 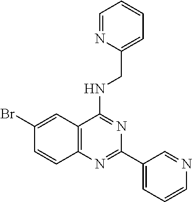 [SMILES: Brc1ccc2nc(-c3cccnc3)nc(NCc3ccccn3)c2c1] | 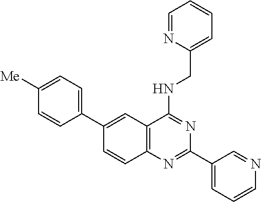 [SMILES: Cc1ccc(-c2ccc3nc(-c4cccnc4)nc(NCc4ccccn4)c3c2)cc1] |
| 381 | 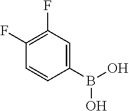 [SMILES: OB(O)c1ccc(F)c(F)c1] | 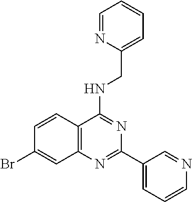 [SMILES: Brc1ccc2c(NCc3ccccn3)nc(-c3cccnc3)nc2c1] | 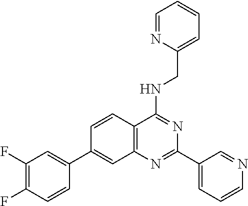 [SMILES: Fc1ccc(-c2ccc3c(NCc4ccccn4)nc(-c4cccnc4)nc3c2)cc1F] |
| 382 | 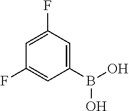 [SMILES: OB(O)c1cc(F)cc(F)c1] | 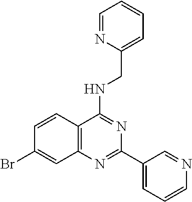 [SMILES: Brc1ccc2c(NCc3ccccn3)nc(-c3cccnc3)nc2c1] | 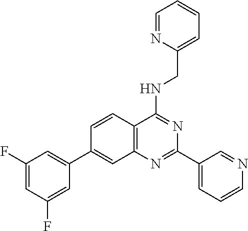 [SMILES: Fc1cc(F)cc(-c2ccc3c(NCc4ccccn4)nc(-c4cccnc4)nc3c2)c1] |
| 383 | 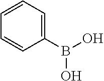 | 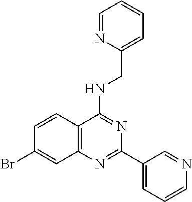 [SMILES: Brc1ccc2c(NCc3ccccn3)nc(-c3cccnc3)nc2c1] | 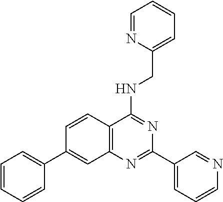 [SMILES: c1ccc(-c2ccc3c(NCc4ccccn4)nc(-c4cccnc4)nc3c2)cc1] |
| 384 | 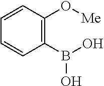 [SMILES: COc1ccccc1B(O)O] | 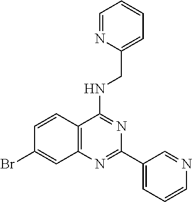 [SMILES: Brc1ccc2c(NCc3ccccn3)nc(-c3cccnc3)nc2c1] | 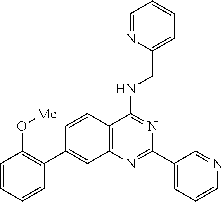 [SMILES: COc1ccccc1-c1ccc2c(NCc3ccccn3)nc(-c3cccnc3)nc2c1] |
| 385 | 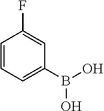 | 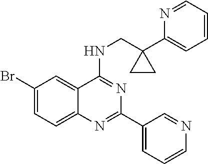 [SMILES: Brc1ccc2nc(-c3cccnc3)nc(NCC3(c4ccccn4)CC3)c2c1] | 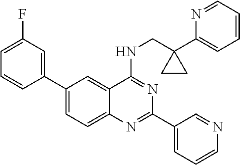 [SMILES: Fc1cccc(-c2ccc3nc(-c4cccnc4)nc(NCC4(c5ccccn5)CC4)c3c2)c1] |
| 386 | 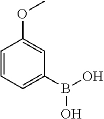 | 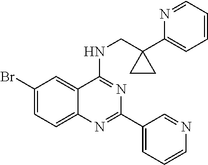 [SMILES: Brc1ccc2nc(-c3cccnc3)nc(NCC3(c4ccccn4)CC3)c2c1] | 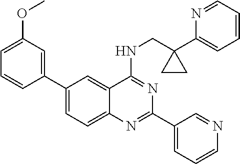 [SMILES: COc1cccc(-c2ccc3nc(-c4cccnc4)nc(NCC4(c5ccccn5)CC4)c3c2)c1] |
| 387 | 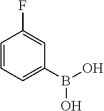 | 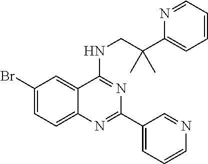 [SMILES: CC(C)(CNc1nc(-c2cccnc2)nc2ccc(Br)cc12)c1ccccn1] | 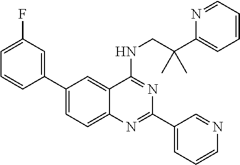 [SMILES: CC(C)(CNc1nc(-c2cccnc2)nc2ccc(-c3cccc(F)c3)cc12)c1ccccn1] |
| 388 | 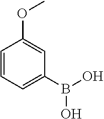 | 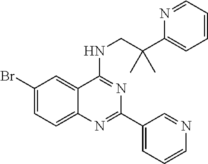 [SMILES: CC(C)(CNc1nc(-c2cccnc2)nc2ccc(Br)cc12)c1ccccn1] | 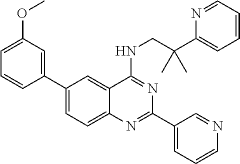 [SMILES: COc1cccc(-c2ccc3nc(-c4cccnc4)nc(NCC(C)(C)c4ccccn4)c3c2)c1] |
| 389 | 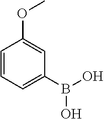 | 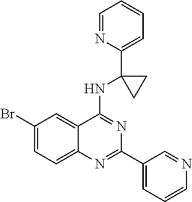 [SMILES: Brc1ccc2nc(-c3cccnc3)nc(NC3(c4ccccn4)CC3)c2c1] | 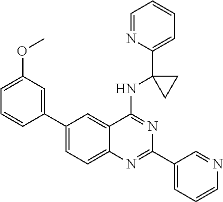 [SMILES: COc1cccc(-c2ccc3nc(-c4cccnc4)nc(NC4(c5ccccn5)CC4)c3c2)c1] |
| 390 | 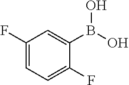 [SMILES: OB(O)c1cc(F)ccc1F] | 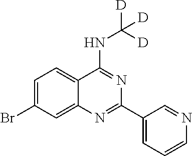 [SMILES: [2H]C([2H])([2H])Nc1nc(-c2cccnc2)nc2cc(Br)ccc12] | 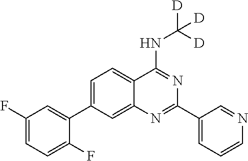 [SMILES: [2H]C([2H])([2H])Nc1nc(-c2cccnc2)nc2cc(-c3cc(F)ccc3F)ccc12] |
| 391 | 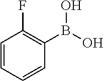 | 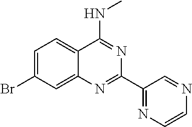 [SMILES: CNc1nc(-c2cnccn2)nc2cc(Br)ccc12] | 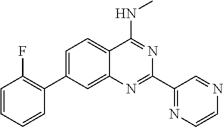 [SMILES: CNc1nc(-c2cnccn2)nc2cc(-c3ccccc3F)ccc12] |
| 392 | 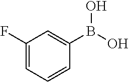 | 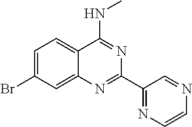 [SMILES: CNc1nc(-c2cnccn2)nc2cc(Br)ccc12] | 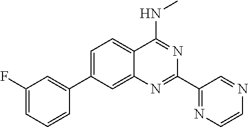 [SMILES: CNc1nc(-c2cnccn2)nc2cc(-c3cccc(F)c3)ccc12] |
| 393 | 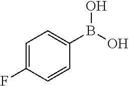 | 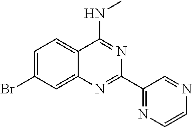 [SMILES: CNc1nc(-c2cnccn2)nc2cc(Br)ccc12] | 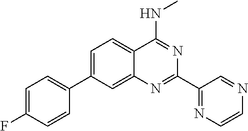 [SMILES: CNc1nc(-c2cnccn2)nc2cc(-c3ccc(F)cc3)ccc12] |
| 394 | 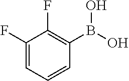 [SMILES: OB(O)c1cccc(F)c1F] | 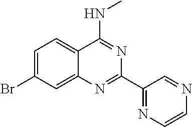 [SMILES: CNc1nc(-c2cnccn2)nc2cc(Br)ccc12] | 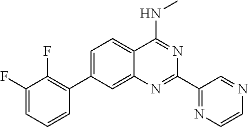 [SMILES: CNc1nc(-c2cnccn2)nc2cc(-c3cccc(F)c3F)ccc12] |
| 395 | 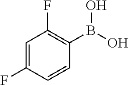 [SMILES: OB(O)c1ccc(F)cc1F] | 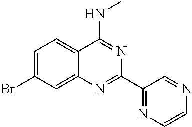 [SMILES: CNc1nc(-c2cnccn2)nc2cc(Br)ccc12] | 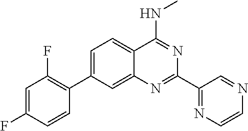 [SMILES: CNc1nc(-c2cnccn2)nc2cc(-c3ccc(F)cc3F)ccc12] |
| 396 | 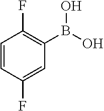 [SMILES: OB(O)c1cc(F)ccc1F] | 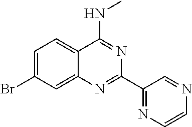 [SMILES: CNc1nc(-c2cnccn2)nc2cc(Br)ccc12] | 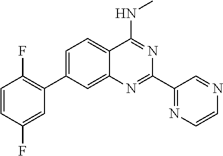 [SMILES: CNc1nc(-c2cnccn2)nc2cc(-c3cc(F)ccc3F)ccc12] |
| 397 | 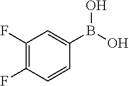 [SMILES: OB(O)c1ccc(F)c(F)c1] | 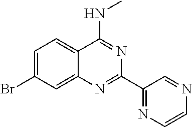 [SMILES: CNc1nc(-c2cnccn2)nc2cc(Br)ccc12] | 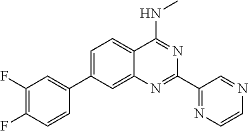 [SMILES: CNc1nc(-c2cnccn2)nc2cc(-c3ccc(F)c(F)c3)ccc12] |
| 398 | 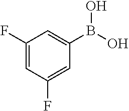 [SMILES: OB(O)c1cc(F)cc(F)c1] | 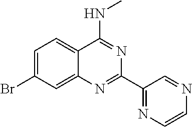 [SMILES: CNc1nc(-c2cnccn2)nc2cc(Br)ccc12] | 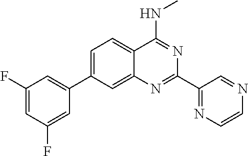 [SMILES: CNc1nc(-c2cnccn2)nc2cc(-c3cc(F)cc(F)c3)ccc12] |
| 399 | 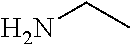 | 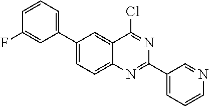 [SMILES: Fc1cccc(-c2ccc3nc(-c4cccnc4)nc(Cl)c3c2)c1] | 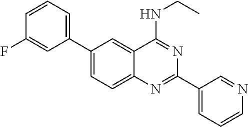 [SMILES: CCNc1nc(-c2cccnc2)nc2ccc(-c3cccc(F)c3)cc12] |
| 400 | 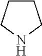 | 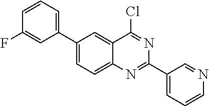 [SMILES: Fc1cccc(-c2ccc3nc(-c4cccnc4)nc(Cl)c3c2)c1] | 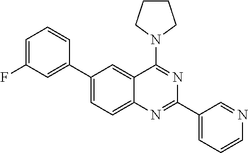 [SMILES: Fc1cccc(-c2ccc3nc(-c4cccnc4)nc(N4CCCC4)c3c2)c1] |
| 401 | 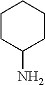 | 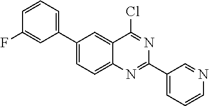 [SMILES: Fc1cccc(-c2ccc3nc(-c4cccnc4)nc(Cl)c3c2)c1] | 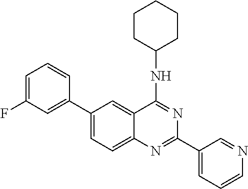 [SMILES: Fc1cccc(-c2ccc3nc(-c4cccnc4)nc(NC4CCCCC4)c3c2)c1] |
| 402 | 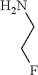 | 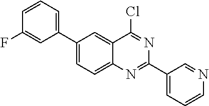 [SMILES: Fc1cccc(-c2ccc3nc(-c4cccnc4)nc(Cl)c3c2)c1] | 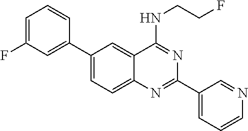 [SMILES: FCCNc1nc(-c2cccnc2)nc2ccc(-c3cccc(F)c3)cc12] |
| 403 | 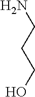 | 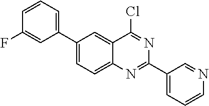 [SMILES: Fc1cccc(-c2ccc3nc(-c4cccnc4)nc(Cl)c3c2)c1] | 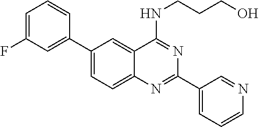 [SMILES: OCCCNc1nc(-c2cccnc2)nc2ccc(-c3cccc(F)c3)cc12] |
| 404 | 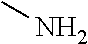 | 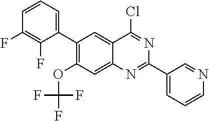 [SMILES: Fc1cccc(-c2cc3c(Cl)nc(-c4cccnc4)nc3cc2OC(F)(F)F)c1F] | 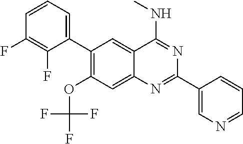 [SMILES: CNc1nc(-c2cccnc2)nc2cc(OC(F)(F)F)c(-c3cccc(F)c3F)cc12] |
| 405 | 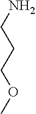 | 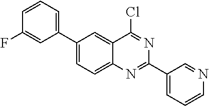 [SMILES: Fc1cccc(-c2ccc3nc(-c4cccnc4)nc(Cl)c3c2)c1] | 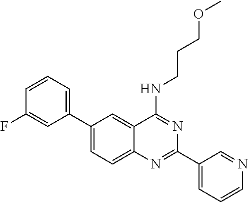 [SMILES: COCCCNc1nc(-c2cccnc2)nc2ccc(-c3cccc(F)c3)cc12] |
| 406 | 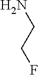 | 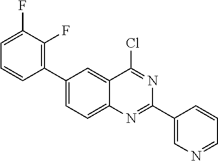 [SMILES: Fc1cccc(-c2ccc3nc(-c4cccnc4)nc(Cl)c3c2)c1F] | 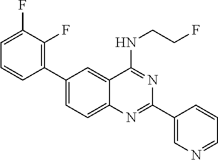 [SMILES: FCCNc1nc(-c2cccnc2)nc2ccc(-c3cccc(F)c3F)cc12] |
| 407 | 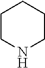 | 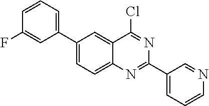 [SMILES: Fc1cccc(-c2ccc3nc(-c4cccnc4)nc(Cl)c3c2)c1] | 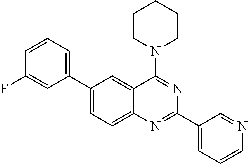 [SMILES: Fc1cccc(-c2ccc3nc(-c4cccnc4)nc(N4CCCCC4)c3c2)c1] |
| 408 | 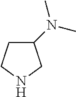 | 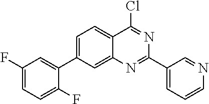 [SMILES: Fc1ccc(F)c(-c2ccc3c(Cl)nc(-c4cccnc4)nc3c2)c1] | 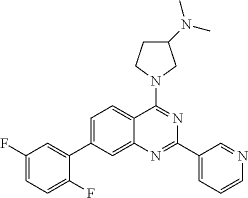 [SMILES: CN(C)C1CCN(c2nc(-c3cccnc3)nc3cc(-c4cc(F)ccc4F)ccc23)C1] |
| 409 | 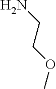 | 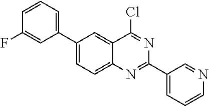 [SMILES: Fc1cccc(-c2ccc3nc(-c4cccnc4)nc(Cl)c3c2)c1] | 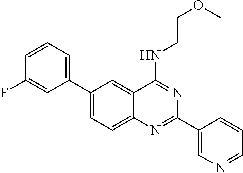 [SMILES: COCCNc1nc(-c2cccnc2)nc2ccc(-c3cccc(F)c3)cc12] |
| 410 | 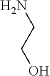 | 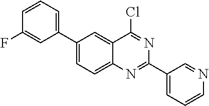 [SMILES: Fc1cccc(-c2ccc3nc(-c4cccnc4)nc(Cl)c3c2)c1] | 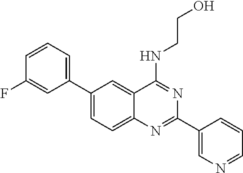 [SMILES: OCCNc1nc(-c2cccnc2)nc2ccc(-c3cccc(F)c3)cc12] |

| | Salt | | ¹H NMR | Purity | Method of | | Retention | LCMS |
|---|---|---|---|---|---|---|---|---|
| Number | type | ¹H NMR | Solvent | percent | Coupling | LCMS | Time | Method |
| 166 | 2 HCl | 1H NMR (300 MHz, DMSO) δ 10.29 | DMSO | 99 | R2 | 370.5 | 1.57 | C |
| | | (s, 1H), 9.62 (d, J = 1.7 Hz, 1H), | | | | (M + 1) | | |
| | | 9.00 (d, J = 7.4 Hz, 1H), 8.94 (dd, | | | | | | |
| | | J = 5.0, 1.6 Hz, 1H), 8.86 (d, J = | | | | | | |
| | | 0.8 Hz, 1H), 8.33 (dd, J = 8.9, 1.1 | | | | | | |
| | | Hz, 1H), 8.19 (d, J = 8.8 Hz, 1H), | | | | | | |
| | | 7.80 (s, 1H), 7.75 (d, J = 7.7 Hz, | | | | | | |
| | | 1H), 7.59 (s, 1H), 7.48 (t, J = 7.6 | | | | | | |
| | | Hz, 1H), 7.34 (d, J = 7.6 Hz, 1H), | | | | | | |
| | | 6.95 (s, 1H), 3.49 (s, 2H), 3.30 (d, | | | | | | |
| | | J = 4.4 Hz, 3H). | | | | | | |
| 167 | 3 HCl | 1H NMR (300 MHz, DMSO) δ 10.01 | DMSO | 99 | R7 | 339.4 | 1.84 | C |
| | | (s, 1H), 9.65 (d, J = 1.7 Hz, 1H), | | | | (M + 1) | | |
| | | 9.17-9.05 (m, 2H), 8.94 (dd, J = | | | | | | |
| | | 5.1, 1.4 Hz, 1H), 8.89 (d, J = 5.3 | | | | | | |
| | | Hz, 1H), 8.60 (d, J = 1.1 Hz, 1H), | | | | | | |
| | | 8.50 (dd, J = 8.6, 1.4 Hz, 1H), | | | | | | |
| | | 8.29 (dd, J = 5.2, 1.9 Hz, 1H), | | | | | | |
| | | 8.15 (dd, J = 8.2, 0.7 Hz, 1H), | | | | | | |
| | | 7.91 (dd, J = 7.7, 4.3 Hz, 1H), | | | | | | |
| | | 3.29 (d, J = 4.3 Hz, 3H). | | | | | | |
| 168 | 2 HCl | 1H NMR (300 MHz, DMSO) δ 10.94 | DMSO | 99 | R7 | 369.4 | 1.52 | C |
| | | (s, 1H), 10.85 (s, 1H), 10.28 (s, | | | | (M + 1) | | |
| | | 1H), 9.63 (s, 1H), 8.99 (d, J = 7.4 | | | | | | |
| | | Hz, 1H), 8.93 (d, J = 4.6 Hz, 1H), | | | | | | |
| | | 1H), 8.19 (d, J = 9.0 Hz, 1H), 7.84 | | | | | | |
| | | (dd, J = 7.9, 4.2 Hz, 1H), 7.45 | | | | | | |
| | | (dd, J = 7.8, 1.3 Hz, 1H), 7.37 (s, | | | | | | |
| | | 1H), 7.07 (d, J = 8.1 Hz, 1H), 3.30 | | | | | | |
| | | (d, J = 4.1 Hz, 3H). | | | | | | |
| 169 | 2 HCl | 1H NMR (300 MHz, DMSO) δ 10.27 | DMSO | 99 | R7 | 384.4 | 2.11 | C |
| | | (s, 1H), 9.64 (d, J = 2.1 Hz, 1H), | | | | (M + 1) | | |
| | | 9.03 (d, J = 7.6 Hz, 1H), 8.99- | | | | | | |
| | | 8.91 (m, 2H), 8.56 (d, J = 1.3 Hz, | | | | | | |
| | | 1H), 8.42 (dd, J = 8.4, 1.4 Hz, | | | | | | |
| | | 1H), 8.21 (d, J = 8.7 Hz, | | | | | | |
| | | 1H), 8.09-7.95 (m, 2H), | | | | | | |
| | | 7.87 (dd, J = 7.6, 5.2 Hz, 1H), | | | | | | |
| | | 3.31 (d, J = 4.4 Hz, 3H), 2.82 (s, 3H). | | | | | | |
| 170 | 2 HCl | 1H NMR (300 MHz, DMSO) δ 10.22 | DMSO | 99 | R7 | 385.5 | 2.21 | C |
| | | (s, 1H), 9.63 (d, J = 1.6 Hz, 1H), | | | | (M + 1) | | |
| | | 9.01 (d, J = 7.9 Hz, 1H), 8.93 (dd, | | | | | | |
| | | J = 5.1, 1.5 Hz, 1H), 8.78 (d, J = | | | | | | |
| | | 1.1 Hz, 1H), 8.33 (dd, J = 8.9, 1.5 | | | | | | |
| | | Hz, 1H), 8.19 (d, J = 8.8 Hz, 1H), | | | | | | |
| | | 7.85 (dd, J = 7.9, 5.3 Hz, 1H), | | | | | | |
| | | 7.55 (d, J = 2.3 Hz, 1H), 7.49 (dd, | | | | | | |
| | | J = 8.4, 2.4 Hz, 1H), 7.11 (d, J = | | | | | | |
| | | 8.3 Hz, 1H), 4.20 (dd, J = 9.9, 4.9 | | | | | | |
| | | Hz, 4H), 3.29 (d, J = 4.5 Hz, 3H), | | | | | | |
| | | 2.15 (dt, J = 10.1, 5.1 Hz, 2H). | | | | | | |
| 171 | 2 HCl | 1H NMR (300 MHz, DMSO) δ 10.23 | DMSO | 96 | R7 | 370.4 | 2.05 | C |
| | | (s, 1H), 9.64 (d, J = 1.7 Hz, 1H), | | | | (M + 1) | | |
| | | 9.49 (s, 1H), 9.12-8.98 (m, 2H), | | | | | | |
| | | 8.94 (d, J = 3.5 Hz, 1H), 8.63 (s, | | | | | | |
| | | 1H), 8.50 (dd, J = 9.1, 1.0 | | | | | | |
| | | Hz, 1H), 8.35 (d, J = 8.5 Hz, 1H), | | | | | | |
| | | 8.23 (d, J = 8.6 Hz, 1H), | | | | | | |
| | | 8.04 (dd, J = 8.3, 1.7 Hz, 1H), | | | | | | |
| | | 7.86 (dd, J = 6.5, 5.9 Hz, 1H),3.31 (d, | | | | | | |
| | | J = 4.3 Hz, 3H). | | | | | | |
| 172 | 2 HCl | 1H NMR (300 MHz, DMSO) δ 10.36 | DMSO | 99 | R7 | 371.5 | 2.29 | C |
| | | (s, 1H), 9.64 (s, 1H), 9.03 (d, J = | | | | (M + 1) | | |
| | | 7.7 Hz, 1H), 8.94 (dd, J = 5.0, 1.5 | | | | | | |
| | | Hz, 1H), 8.90 (s, 1H), 8.36 (dd, J = | | | | | | |
| | | 8.8, 1.3 Hz, 1H), 8.23 (d, J = | | | | | | |
| | | 8.9 Hz, 1H), 7.94-7.80 (m, 3H), | | | | | | |
| | | 7.48 (d, J = 8.2 Hz, 2H), 4.52 (s, | | | | | | |
| | | 2H), 3.51 (q, J = 7.0 Hz, 2H), 3.30 | | | | | | |
| | | (d, J = 4.3 Hz, 3H), 1.17 (t, J = | | | | | | |
| | | 7.0 Hz, 3H). | | | | | | |
| 173 | 2 HCl | 1H NMR (300 MHz, DMSO) δ 10.17 | DMSO | 96 | R7 | 343.1 | 1.78 | C |
| | | (s, 1H), 9.61 (d, J = 1.6 Hz, 1H), | | | | (M + 1) | | |
| | | 8.96 (dd, J = 17.6, 7.6 Hz, 2H), | | | | | | |
| | | 8.83 (s, 1H), 8.36 (d, J = 8.6 Hz, | | | | | | |
| | | 1H), 8.14 (d, J = 9.3 Hz, 1H), 7.86 | | | | | | |
| | | (d, J = 8.1 Hz, 3H), 7.49 (d, J = | | | | | | |
| | | 8.1 Hz, 2H), 4.57 (s, 2H), 3.30 (d, | | | | | | |
| | | J = 4.1 Hz, 3H). | | | | | | |
| 174 | 2 HCl | 1H NMR (300 MHz, DMSO) δ 10.28 | DMSO | 99 | R7 | 413.3 | 2.56 | C |
| | | (s, 1H), 9.64 (d, J = 1.4 Hz, 1H), | | | | (M + 1) | | |
| | | 9.06 (d, J = 8.0 Hz, 1H), 9.00- | | | | | | |
| | | 8.91 (m, 2H), 8.40 (d, J = 8.7 Hz, | | | | | | |
| | | 1H), 8.22 (d, J = 8.8 Hz, 1H), 8.05 | | | | | | |
| | | (d, J = 8.4 Hz, 2H), 7.95-7.83 | | | | | | |
| | | (m, 3H), 3.29 (d, J = 4.3 Hz, 3H). | | | | | | |
| 175 | 2 HCl | 1H NMR (300 MHz, DMSO) δ 10.34 | DMSO | 99 | R7 | 395.4 | 2.43 | C |
| | | (s, 1H), 9.68 (d, J = 1.4 Hz, 1H), | | | | (M + 1) | | |
| | | 9.10 (d, J = 7.7 Hz, 1H), 8.96 (dd, | | | | | | |
| | | J = 5.1, 1.5 Hz, 1H), 8.64 (s, 1H), | | | | | | |
| | | 8.30 (d, J = 8.6 Hz, 1H), 8.06 (d, J = | | | | | | |
| | | 8.6 Hz, 1H), 7.90 (dd, J = 7.8, | | | | | | |
| | | 5.2 Hz, 1H), 7.77 (s, 1H), 7.69 (d, | | | | | | |
| | | J= 8.0 Hz, 1H), 7.55 (d, J = 8.0 | | | | | | |
| | | Hz, 1H), 3.26 (d, J = 4.0 Hz, 3H), | | | | | | |
| | | 2.39 (s, 3H). | | | | | | |
| 176 | 2 HCl | 1H NMR (300 MHz, DMSO) δ 10.23 | DMSO | 99 | R8 | 320.4 | 1.88 | C |
| | | (s, 1H), 9.64 (d, J = 1.7 Hz, 1H), | | | | (M + 1) | | |
| | | 9.49 (s, 1H), 9.12-8.98 (m, 2H), | | | | | | |
| | | 8.94 (d, J = 3.5 Hz, 1H), 8.63 (s, | | | | | | |
| | | 1H), 8.50 (dd, J = 9.1, 1.0 Hz, | | | | | | |
| | | 1H), 8.35 (d, J = 8.5 Hz, 1H), 8.23 | | | | | | |
| | | (d, J = 8.6 Hz, 1H), 8.04 (dd, J = | | | | | | |
| | | 8.3, 1.7 Hz, 1H), 7.86 (dd, J = 6.5, | | | | | | |
| | | 5.9 Hz, 1H), 3.31 (d, J = 4.3 Hz, 3H). | | | | | | |
| 177 | 2 HCl | 1H NMR (300 MHz, DMSO) δ 10.23 | DMSO | 99 | R8 | 320.1 | 1.70 | C |
| | | (s, 1H), 9.64 (d, J = 1.7 Hz, 1H), | | | | (M + 1) | | |
| | | 9.49 (s, 1H), 9.12-8.98 (m, 2H), | | | | | | |
| | | 8.94 (d, J = 3.5 Hz, 1H), 8.63 (s, | | | | | | |
| | | 1H), 8.50 (dd, J = 9.1, 1.0 Hz, | | | | | | |
| | | 1H), 8.35 (d, J = 8.5 Hz, 1H), 8.23 | | | | | | |
| | | (d, J= 8.6 Hz, 1H), 8.04 (dd, J = | | | | | | |
| | | 8.3, 1.7 Hz, 1H), 7.86 (dd, J = 6.5, | | | | | | |
| | | 5.9 Hz, 1H), 3.31 (d, J = 4.3 Hz, 3H). | | | | | | |
| 178 | 2 HCl | 1H NMR (300 MHz, DMSO) δ 10.11 | DMSO | 99 | R8 | 304.2 | 1.74 | C |
| | | (s, 1H),9.66 (d, J = 1.7 Hz, 1H), | | | | (M + 1) | | |
| | | 9.16 (d, J = 7.8 Hz, 1H), 9.04 (s, | | | | | | |
| | | 1H), 8.97 (d, J = 4.6 Hz, 1H), 8.47 | | | | | | |
| | | (d, J = 9.1 Hz, 1H), 8.37 (s, 1H), | | | | | | |
| | | 8.24 (d, J = 8.5 Hz, 1H), 7.94 (dd, | | | | | | |
| | | J = 7.4, 5.5 Hz, 1H), 7.49 (s, 1H), | | | | | | |
| | | 3.25 (d, J = 4.1 Hz, 3H). | | | | | | |
| 179 | 2 HCl | 1H NMR (300 MHz, DMSO) δ 9.65 | DMSO | 98 | R2 | 359.1 | 2.19 | C |
| | | (d, J = 1.4 Hz, 1H), 8.84-8.74 | | | | (M + 1) | | |
| | | (m, 1H), 8.69 (dd, J = 4.7, 1.7 | | | | | | |
| | | Hz, 1H), 8.57 (d, J = 4.3 Hz, 1H), | | | | | | |
| | | 8.38 (d, J = 1.6 Hz, 1H), 7.95 (dd, | | | | | | |
| | | J = 8.6, 1.8 Hz, 1H), 7.85 (d, J = | | | | | | |
| | | 8.6 Hz,1H), 7.55 (dd, J = 7.9, 4.8 | | | | | | |
| | | Hz, 1H), 7.44-7.22 (m, 3H), 3.72 | | | | | | |
| | | (s, 3H), 3.16 (d, J = 4.4 Hz, 3H). | | | | | | |
| 180 | | 1H NMR (300 MHz, DMSO) δ 9.65 | DMSO | 98 | R2 | 388.3 | 1.75 | C |
| | | (s, 1H), 8.78 (d, J = 8.0 Hz, 1H), | | | | (M + 1) | | |
| | | 8.73-8.60 (m, 3H), 8.44 (s, 1H), | | | | | | |
| | | 8.16 (d, J = 8.7 Hz, 1H), 8.14- | | | | | | |
| | | 8.06 (m, 1H), 7.98 (dd, J = 5.7, | | | | | | |
| | | 2.8 Hz, 1H), 7.87 (d, J = 8.7 Hz, | | | | | | |
| | | 1H), 7.60-7.43 (m, 2H), 3.19 (d, | | | | | | |
| | | J = 4.2 Hz, 3H), 2.83 (d, J = 4.5 | | | | | | |
| | | Hz, 3H). | | | | | | |
| 181 | | 1H NMR (300 MHz, DMSO) δ 9.63 | DMSO | 98 | R2 | 402.2 | 1.81 | C |
| | | (d, J = 1.4 Hz, 1H), 8.82-8.72 | | | | (M + 1) | | |
| | | (m, 1H), 8.72-8.57 (m, 3H), 8.19 | | | | | | |
| | | (dd, J = 8.8, 1.8 Hz, 1H), 7.89- | | | | | | |
| | | 7.75 (m, 3H), 7.60-7.50 (m, 2H), | | | | | | |
| | | 3.17 (d, J = 4.3 Hz, 3H), 3.03 (s, | | | | | | |
| | | 3H), 2.92 (d, J = 10.3 Hz, 3H). | | | | | | |
| 182 | 2HCl | 1H NMR (300 MHz, DMSO) δ 10.39 | DMSO | 98 | R2 | 362.4 | 2.11 | C |
| | | (s, 1H), 9.71 (s, 1H), 9.14 (d, J = | | | | (M + 1) | | |
| | | 8.1 Hz, 1H), 8.98 (d, J = 4.0 Hz, | | | | | | |
| | | 1H), 8.71 (s, 1H), 8.31 (d, J = 8.8- | | | | | | |
| | | 7.89 (m, 1H), 7.38 (dd, J = 9.2, | | | | | | |
| | | 3.0 Hz, 1H), 7.34-7.14 (m, 2H), | | | | | | |
| | | 3.81 (s, 3H), 3.30 (d, J = 4.4 Hz, 3H). | | | | | | |
| 183 | | 1H NMR (300 MHz, DMSO) δ 10.39 | DMSO | 98 | R2 | 374.1 | 1.67 | C |
| | | (s, 1H), 9.71 (s, 1H), 9.14 (d, J = | | | | (M + 1) | | |
| | | 8.1 Hz, 1H), 8.98 (d, J = 4.0 Hz, | | | | | | |
| | | 1H), 8.71 (s, 1H), 8.31 (d, J = 8.8 | | | | | | |
| | | Hz, 1H), 8.25-8.19 (m, 1H), 7.96- | | | | | | |
| | | 7.89 (m, 1H), 7.38 (dd, J = 9.2, | | | | | | |
| | | 3.0 Hz, 1H), 7.34-7.14 (m, 2H), | | | | | | |
| | | 3.81 (s, 3H), 3.30 (d, J = 4.4 Hz, 3H). | | | | | | |
| 184 | | 1H NMR (300 MHz, DMSO) δ 8.77 | DMSO | 98 | R10 | 439.0 | 2.13 | C |
| | | (d, J = 7.6 Hz, 1H), 8.62 (d, J = | | | | (M + 1) | | |
| | | 4.2 Hz, 1H), 8.31 (d, J = 1.6 Hz, | | | | | | |
| | | 1H), 8.05 (dd, J = 8.8, 1.8 Hz, | | | | | | |
| | | 1H), 7.83 (d, J = 8.7 Hz, 1H), | | | | | | |
| | | 7.57 (s, 1H), 3.85-3.65 (m, 3H), | | | | | | |
| | | 3.44 (dd, J = 14.9, 10.5 Hz, 4H), | | | | | | |
| | | 3.16 (d, J = 4.2 Hz, 2H). | | | | | | |
| 185 | | 1H NMR (300 MHz, DMSO) δ 10.39 | DMSO | 98 | R2 | 415.4 | 2.46 | C |
| | | (s, 1H), 9.71 (s, 1H), 9.14 (d, J = | | | | (M + 1) | | |
| | | 8.1 Hz, 1H), 8.98 (d, J = 4.0 Hz, | | | | | | |
| | | 1H), 8.71 (s, 1H), 8.31 (d, J = 8.8 | | | | | | |
| | | Hz, 1H), 8.25-8.19 (m, 1H), 7.96- | | | | | | |
| | | 7.89 (m, 1H), 7.38 (dd, J = 9.2, | | | | | | |
| | | 3.0 Hz, 1H), 7.34-7.14 (m, 2H), | | | | | | |
| | | 3.81 (s, 3H), 3.30 (d, J = 4.4 Hz, 3H). | | | | | | |
| 186 | | | DMSO | 98 | R2 | 379.1 | 2.23 | C |
| | | | | | | (M + 1) | | |
| 187 | | | DMSO | 98 | R2 | 414.2 | 1.80 | C |
| | | | | | | (M + 1) | | |
| 188 | | | DMSO | 98 | R2 | 357.1 | 1.34 | C |
| | | | | | | (M + 1) | | |
| 189 | Na | 1H NMR (300 MHz, DMSO) δ 9.78 | DMSO | 98 | R2 | 357.2 | 1.37 | C |
| | | (s, 1H), 9.60 (d, J = 1.6 Hz, 1H), | | | | (M + 1) | | |
| | | 8.93 (d, J = 8.3 Hz, 1H), 8.90- | | | | | | |
| | | 8.78 (m, 2H), 8.32 (dd, J = 8.7, | | | | | | |
| | | 1.6 Hz, 1H), 8.07 (d, J = 8.5 Hz, | | | | | | |
| | | 3H), 7.99 (d, J = 8.4 Hz, 2H), 7.76 | | | | | | |
| | | (dd, J = 7.8, 5.0 Hz, 1H), 3.25 (d, | | | | | | |
| | | J = 4.3 Hz, 3H). | | | | | | |
| 190 | | | DMSO | 98 | R2 | 384.2 | 1.67 | C |
| | | | | | | (M + 1) | | |
| 191 | 3 HCl | ¹H-NMR (400 MHz, MeOD): | CD3OD | 95 | R7 | 345.9 | t = 1.232 min | Method A |
| | | δ 9.84 (d, J = 1.7 Hz, | | | | (M + 1) | | (0.01% TFA) |
| | | 1H), 9.44 (d, J = 8.3 Hz, 1H), | | | | | | |
| | | 9.16 (d, J = 4.7 Hz, 1H), 8.79 (s, | | | | | | |
| | | 1H), 8.35-8.27 (m, 2H), 8.22 | | | | | | |
| | | (d, J = 8.7 Hz, 1H), 7.81 (dd, | | | | | | |
| | | J = 6.4, 2.4 Hz, 1H), 7.62-7.52 | | | | | | |
| | | (m, 2H), 3.50 (s, 3H). | | | | | | |
| 192 | 2HCl | ¹H-NMR (400 MHz, MeOD): | MeOD | 95 | R6 | 389.0 | t = 1.859 min | Method B |
| | | δ 9.74 (s, 1H), 9.38 (t, J = 10.3 Hz, | | | | (M + 1) | | (NH4—HCO3) |
| | | 1H), 9.07 (d, J = 5.2 Hz, 1H), | | | | | | |
| | | 8.46 (s, 1H), 8.30-8.20 (m, 1H), | | | | | | |
| | | 8.17 (d, J = 8.7 Hz, 1H), 8.09- | | | | | | |
| | | 8.02 (m, 1H), 7.50 (t, J = 8.9 Hz, | | | | | | |
| | | 1 H), 6.84 (d, J = 8.6 Hz, 1H), 6.78 | | | | | | |
| | | (d, J = 13.1 Hz, 1H), 3.92 (t, J = | | | | | | |
| | | 6.4 Hz, 2H), 3.37 (s, 3H), 1.79-1.67 | | | | | | |
| | | (m, 2H), 0.97 (t, J = 7.4 Hz, 3H). | | | | | | |
| 193 | 2HCl | ¹H-NMR (400 MHz, DMSO-d₆): δ | DMSO | 95 | R6 | 459.1 | t = 2.032 min | Method B |
| | | 9.66 (d, J = 1.4 Hz, 1H), 8.80 (dt, | | | | (M + 1) | | (NH4—HCO3) |
| | | J = 7.9, 1.8 Hz, 1H), 8.70 (dd, J = | | | | | | |
| | | 4.7, 1.5 Hz, 1H), 8.56 (d, J = 4.5 | | | | | | |
| | | Hz, 1H), 8.31 (s, 1H), 8.13 (dd, J = | | | | | | |
| | | 8.1, 1.6 Hz, 1H), 7.96 (d, J = 1.6 | | | | | | |
| | | Hz, 1H), 7.89 (d, J = 5.4 Hz, 3H), | | | | | | |
| | | 7.56 (dd, J = 7.9, 4.8 Hz, 1H), 3.17, | | | | | | |
| | | 3.17(d, J = 4.4 Hz, 3H), 2.48 (s, 3H). | | | | | | |
| 194 | 2HCl | ¹H-NMR (400 MHz, DMSO-d₆): δ | DMSO | 95 | R6 | 379.7 | t = 1.853 min | Method B |
| | | 9.61 (s, 1H), 9.26 (dd, J = 5.1, 2.3 | | | | (M + 1) | | (NH4—HCO3) |
| | | Hz, 1H), 9.18-9.09 (m, 1H), 9.00 | | | | | | |
| | | (d, J = 4.9 Hz, 1H), 8.21-8.03 (m, | | | | | | |
| | | 2H), 7.65-7.33 (m, 4H), 4.06 (s, | | | | | | |
| | | 3H), 3.21 (s, 3H). | | | | | | |
| 195 | 2HCl | ¹H-NMR (400 MHz, DMSO-d₆): δ | DMSO | 95 | R6 | 379.7 | t = 1.247 min | Method A |
| | | 9.58 (s, 1H), 9.34-9.17 (m, 2H), | | | | (M + 1) | | (TFA) |
| | | 9.01 (t, J = 4.2 Hz, 1H), 8.40- | | | | | | |
| | | 8.32 (m, 1H), 8.11 (t, J = 5.4 Hz, | | | | | | |
| | | 2H), 7.79 (d, J = 6.7 Hz, 1H), 7.51 | | | | | | |
| | | (d, J = 20.2 Hz, 1H), 7.31 (s, 1H), | | | | | | |
| | | 4.07 (d, J = 5.7 Hz, 3H), 3.21 (d, J = | | | | | | |
| | | 8.8 Hz, 3H). | | | | | | |
| 196 | 2HCl | ¹H-NMR (400 MHz, CD₃OD): δ | CD3OD | 95 | R6 | 367.0 | t = 1.707 min | Method B |
| | | 9.87 (s, 1H), 9.51 (d, J = 8.4 Hz, | | | | (M + 1) | | (NH4—HCO3) |
| | | 1H), 9.18 (d, J = 5.2 Hz, 1H), 8.56 | | | | | | |
| | | (d, J = 7.2 Hz, 1H), 8.35 (dd, J = | | | | | | |
| | | 7.6, 1.6 Hz, 1H), 7.92 (d, J = 11.2 | | | | | | |
| | | Hz, 1H), 7.66 (d, J = 6.4 Hz, 1H), | | | | | | |
| | | 7.26-7.20 (m, 2H), 3.47 (s, 3H). | | | | | | |
| 197 | 2HCl | ¹H-NMR (400 MHz, MeOD): δ 9.80 | MeOD | 95 | R6 | 417.0 | t = 1.755 min | Method B |
| | | (s, 1H), 9.50 (d, J = 8.2 Hz, 1H), | | | | (M + 1) | | (NH4—HCO3) |
| | | 9.05 (d, J = 5.3 Hz, 1H), 8.41 (s, | | | | | | |
| | | 1H), 8.33 (s, 1H), 8.25 (dd, J = 8.1, | | | | | | |
| | | 5.7 Hz, 1H), 7.41-7.33 (m, 1H), | | | | | | |
| | | 7.26-7.19 (m, 1H), 7.14 (t, J = | | | | | | |
| | | 6.6 Hz, 1H), 3.32 (s, 3H). | | | | | | |
| 198 | 2HCl | ¹H-NMR (400 MHz, MeOD): δ 9.81 | MeOD | 95 | R6 | 417.0 | t = 1.833 min | Method B |
| | | (s, 1H), 9.50 (d, J = 8.2 Hz, 1H), | | | | (M + 1) | | (NH4—HCO3) |
| | | 9.06 (d, J = 5.5 Hz, 1H), 8.43 (s, | | | | | | |
| | | 1H), 8.32 (s, 1H), 8.27 (dd, J = 8.1, | | | | | | |
| | | 5.8 Hz, 1H), 7.38 (dd, J = 14.9, 8.2 | | | | | | |
| | | Hz, 1H), 7.12-7.02 (m, 2H), 3.33 | | | | | | |
| | | (s, 3H). | | | | | | |
| 199 | | ¹H-NMR (400 MHz, MeOD): δ 9.53 | MeOD | 95 | R6 | 458.8 | t = 1.748 min | Method B |
| | | (d, J = 1.5 Hz, 1H), 8.81 (td, J = | | | | (M + 1) | | (NH4—HCO3) |
| | | 8.0, 1.8 Hz, 1H), 8.55 (dd, J = 4.9, | | | | | | |
| | | 1.5 Hz, 1H), 8.30 (d, J = 1.4 Hz, | | | | | | |
| | | 1H), 8.21 (dd, J = 8.0, 1.6 Hz, 1H), | | | | | | |
| | | 8.01 (d, J = 1.5 Hz, 1H), 7.82 (d, J = | | | | | | |
| | | 8.6 Hz, 1H), 7.68 (dd, J = 13.7, | | | | | | |
| | | 4.4 Hz, 2H), 7.50 (dd, J = 7.7, 5.0 | | | | | | |
| | | Hz, 1H), 3.16 (d, J = 7.8 Hz, 6H). | | | | | | |
| 200 | | ¹H-NMR (400 MHz, DMSO-d₆): δ | DMSO | 95 | R6 | 410.1 | t = 1.905 min | Method B |
| | | 9.65 (d, J = 1.7 Hz, 1H), 8.79 (d, J = | | | | (M + 1) | | (NH4—HCO3) |
| | | 8.0 Hz, 1H), 8.69 (d, J = 5.5 Hz, | | | | | | |
| | | 3H), 8.20 (dd, J = 8.7, 1.5 Hz, 1H), | | | | | | |
| | | 8.01-7.96 (m, 1H), 7.99 (s, 3H), | | | | | | |
| | | 7.87 (d, J = 8.7 Hz, 1H), 7.55 (dd, | | | | | | |
| | | J = 7.9, 4.8 Hz, 1H), 4.15 (s, 2H), | | | | | | |
| | | 3.20 (d, J = 4.3 Hz, 3H), 1.32 (s, 6H). | | | | | | |
| 201 | 2HCl | ¹H-NMR (400 MHz, DMSO-d₆): δ | DMSO | 95 | R6 | 375.1 | t = 1.989 min | Method B |
| | | 10.55 (s, 1H), 9.73 (s, 1H), 9.18 (d, | | | | (M + 1) | | (NH4—HCO3) |
| | | J = 7.9 Hz, 1H), 9.00 (d, J = 4.6 | | | | | | |
| | | Hz, 1H), 8.85 (s, 1H), 8.35 (d, J = | | | | | | |
| | | 8.6 Hz, 1H), 8.21 (d, J = 8.7 Hz, | | | | | | |
| | | 1H), 7.94 (dd, J = 7.7, 5.2 Hz, 1H), | | | | | | |
| | | 7.37-7.27 (m, 1H), 7.24 (dd, J = | | | | | | |
| | | 6.4, 3.0 Hz, 1H), 7.03 (dt, J = 8.8, | | | | | | |
| | | 3.3 Hz, 1H), 4.14 (q, J = 6.9 Hz, | | | | | | |
| | | 2H), 3.30 (d, J = 4.3 Hz, 3H), 1.36 | | | | | | |
| | | (t, J = 6.9 Hz, 3H). | | | | | | |
| 202 | 2HCl | ¹H-NMR (400 MHz, DMSO-d₆): δ | DMSO | 95 | R6 | 449.1 | t = 2.066 min | Method B |
| | | 10.24 (s, 1H), 9.72 (s, 1H), 9.19 (d, | | | | (M + 1) | | (NH4—HCO3) |
| | | J = 7.8 Hz, 1H), 9.01 (d, J = 4.4 | | | | | | |
| | | Hz, 1H), 8.64 (s, 1H), 8.33 (d, J = | | | | | | |
| | | 8.4 Hz, 1H), 8.26 (d, J = 6.5 Hz, | | | | | | |
| | | 2H), 8.01 (d, J = 8.7 Hz, 1H), 7.97 | | | | | | |
| | | (dd, J = 7.6, 5.4 Hz, 1H), 7.84 (d, J = | | | | | | |
| | | 8.4 Hz, 1 H), 3.27 (d, J = 4.3 Hz, 3H). | | | | | | |
| 203 | 2 HCl | ¹H-NMR (400 MHz, DMSO-d₆): δ | DMSO | 95 | R6 | 382.1 | t = 1.044 min | Method A |
| | | 10.55 (s, 1H), 10.41 (s, 1H), 9.70 | | | | (M + 1) | | (0.01% TFA) |
| | | (s, 1H), 9.12 (s, 1H), 8.98 (s, 1H), | | | | | | |
| | | 8.85 (s, 1H), 8.45-8.18 (m, 3H), | | | | | | |
| | | 7.91 (s, 1 H), 7.72 (d, J = 7.6 Hz, | | | | | | |
| | | 1H), 7.60 (d, J = 7.4 Hz, 1H), 7.51 | | | | | | |
| | | (t, J = 7.5 Hz, 1H), 6.56 (dd, J = | | | | | | |
| | | 16.8, 10.2 Hz, 1H), 6.30 (d, J = | | | | | | |
| | | 16.9 Hz, 1H), 5.80 (d, J = 10.8 Hz, | | | | | | |
| | | 1H), 3.31 (d, J = 3.7 Hz, 3H). | | | | | | |
| 204 | 2HCl | ¹H-NMR (400 MHz, DMSO-d₆): δ | DMSO | 95 | R6 | 379.0 | t = 1.696 min | Method B |
| | | 9.58 (s, 1H), 9.20 (d, J = 7.6 Hz, | | | | (M + 1) | | (NH4—HCO3) |
| | | 2H), 8.97 (d, J = 5.0 Hz, 1H), 8.34 | | | | | | |
| | | (s, 1H), 8.04 (dd, J = 7.6, 5.7 Hz, | | | | | | |
| | | 1H), 7.75 (d, J = 7.2 Hz, 2H), 7.72 | | | | | | |
| | | (s, 1H), 7.35-7.33 (m, 1H), 4.12 | | | | | | |
| | | (s, 3H), 3.22 (d, J = 4.0 Hz, 3H). | | | | | | |
| 205 | 2HCl | ¹H-NMR (400 MHz, DMSO-d₆): δ | DMSO | 95 | R6 | 368.1 | t = 1.564 min | Method B |
| | | 9.64 (s, 1H), 9.01 (d, J = 7.9 Hz, | | | | (M + 1) | | (NH4—HCO3) |
| | | 1H), 8.82 (d, J = 13.1 Hz, 2H), | | | | | | |
| | | 8.07 (s, 1H), 8.04 (d, J = 7.7 Hz, | | | | | | |
| | | 1H), 7.89 (t, J = 7.6 Hz, 1H), 7.81 | | | | | | |
| | | (d, J = 7.5 Hz, 2H), 7.67 (t, J = 7.5 | | | | | | |
| | | Hz, 1H), 7.57 (s, 1H), 4.06 (s, 3H), | | | | | | |
| | | 3.18 (d, J = 4.2 Hz, 3H). | | | | | | |
| 206 | 2HCl | ¹H-NMR (400 MHz, MeOD): δ | MeOD | 95 | R6 | 368.1 | t = 1.593 min | Method B |
| | | 9.60 (s, 1H), 9.22 (d, J = 8.1 Hz, | | | | (M + 1) | | (NH4—HCO3) |
| | | 1H), 9.06 (d, J = 5.3 Hz, 1H), 8.22 | | | | | | |
| | | (dd, J = 7.8, 6.1 Hz, 1H), | | | | | | |
| | | 8.18 (d, J = 5.4 Hz, 2H), 8.10 | | | | | | |
| | | (d, J = 7.8 Hz, 1H), 7.83 (s, 1H), | | | | | | |
| | | 7.75 (d, J = 7.7 Hz, 1H), 7.66 (t, | | | | | | |
| | | J = 7.8 Hz, 1H), 4.18 (s, 3H), | | | | | | |
| | | 3.36 (s, 3H). | | | | | | |
| 207 | 2HCl | ¹H-NMR (400 MHz, MeOD): δ 9.73 | MeOD | 95 | R6 | 373.1 | t = 1.808 min | Method B |
| | | (s, 1H), 9.36 (d, J = 6.7 Hz, 1H), | | | | (M + 5) | | (NH4—HCO3) |
| | | 9.20 (d, J = 4.4 Hz, 1 H), 8.37 (s, | | | | | | |
| | | 1H), 8.07 (s, 1H), 7.83 (s, 1H), | | | | | | |
| | | 7.51 (d, J = 7.7 Hz, 1H), 7.47 (d, J = | | | | | | |
| | | 7.7 Hz, 1H), 7.20 (d, J = 8.3 Hz, | | | | | | |
| | | 1H), 7.14 (t, J = 7.4 Hz, 1H), 4.21 | | | | | | |
| | | (s, 3H), 3.90 (s, 3H), 3.45 (s, 3H). | | | | | | |
| 208 | 2HCl | ¹H-NMR (400 MHz, MeOD): δ 9.52 | MeOD | 95 | R6 | 371.1 | t = 1.810 min | Method B |
| | | (s, 1H), 8.98 (d, J = 8.0 Hz, 2H), | | | | (M + 4) | | (NH4—HCO3) |
| | | 8.11 (d, J = 1.4 Hz, 1H), 8.00 (dd, | | | | | | |
| | | J = 7.9, 5.4 Hz, 1H), 7.84-7.76 | | | | | | |
| | | (m, 3H), 7.10 (d, J = 8.8 Hz, 2H), | | | | | | |
| | | 4.25 (s, 3H), 3.89 (s, 3H), 3.44 (s, 3H). | | | | | | |
| 209 | 2HCl | ¹H-NMR (400 MHz, DMSO-d₆): δ | DMSO | 96 | R6 | 415.0 | t = 1.877 min | Method B |
| | | 9.70-9.65 (m, 2H), 9.29 (d, J = | | | | (M + 1) | | (NH4—HCO3) |
| | | 7.9 Hz, 1H), 9.01 (d, J = 5.3 Hz, | | | | | | |
| | | 1H), 8.71 (s, 1H), 8.08 (s, 1H), | | | | | | |
| | | 8.04 (dd, J = 7.6, 5.7 Hz, 1H), 7.65- | | | | | | |
| | | 7.58 (m, 1H), 7.48 (t, J = 8.3 Hz, | | | | | | |
| | | 2H), 7.39-7.33 (m, 1H), 3.24 (d, J = | | | | | | |
| | | 4.4 Hz, 3H). | | | | | | |
| 210 | | ¹H-NMR (400 MHz, DMSO-d₆): δ | DMSO | 95 | R6 | 332.0 | t = 1.696 min | Method B |
| | | 9.71 (s, 2H), 9.31 (s, 1 H), 8.74 (d, | | | | (M + 1) | | (NH4—HCO3) |
| | | J = 4.4 Hz, 1H), 8.65 (d, J = 2.0 | | | | | | |
| | | Hz, 1H), 8.21 (dd, J = 8.6, 1.8 Hz, | | | | | | |
| | | 1H), 7.88 (d, J = 8.8 Hz, 1H), 7.73 | | | | | | |
| | | (d, J = 8.0 Hz, 2H), 7.59 (q, J = | | | | | | |
| | | 7.4 Hz, 1H), 7.27 (dd, J = 9.0, 7.4 Hz, | | | | | | |
| | | 1H), 3.20 (d, J = 4.4 Hz, 3H). | | | | | | |
| 211 | 2HCl | ¹H-NMR (400 MHz, CD₃OD-d₆): δ | CD3OD | 95 | R6 | 361.7 | t = 1.144 min | Method A |
| | | 9.77 (s, 1H), 9.31 (d, J = 8.3 Hz, | | | | (M + 1) | | (TFA) |
| | | 1H), 9.12 (d, J = 4.9 Hz, 1H), 8.36 | | | | | | |
| | | (s, 1H), 8.21 (dd, J = 8.1, 5.5 Hz, | | | | | | |
| | | 1H), 7.60 (s, 1H), 7.57-7.49 (m, | | | | | | |
| | | 1H), 7.46 (d, J = 7.8 Hz, 1H), 7.42 | | | | | | |
| | | (dt, J = 10.0, 2.1 Hz, 1H), 7.21 (td, | | | | | | |
| | | J = 8.4, 2.0 Hz, 1H), 4.11 (s, 3H), | | | | | | |
| | | 3.44 (s, 3H). | | | | | | |
| 212 | 2HCl | ¹H-NMR (400 MHz, DMSO-d₆): δ | DMSO | 96 | R6 | 332.0 | t = 1.810 min | Method B |
| | | 10.13 (s, 1H), 9.45 (d, J = 4.4 Hz, | | | | (M + 1) | | (NH4—HCO3) |
| | | 1H), 8.94 (s, 1H), 8.70 (s, 1H), | | | | | | |
| | | 8.54 (dd, J = 5.3, 2.2 Hz, 1H), 8.26 | | | | | | |
| | | (dd, J = 8.7, 1.7 Hz, 1H), 7.94 (d, J = | | | | | | |
| | | 8.7 Hz, 1H), 7.75 (d, J = 7.9 Hz, | | | | | | |
| | | 2H), 7.60 (dd, J = 14.4, 7.6 Hz, | | | | | | |
| | | 1H), 7.28 (dd, J = 9.7, 7.7 Hz, 1H), | | | | | | |
| | | 3.23 (d, J = 4.4 Hz, 3H). | | | | | | |
| 213 | | ¹H-NMR (400 MHz, DMSO-d₆): δ | DMSO | 97 | R6 | 318.1 | t = 1.734 min | Method B |
| | | 9.63 (s, 2H), 9.30 (s, 1H), 8.70 (d, | | | | (M + 1) | | (NH4—HCO3) |
| | | J = 1.6 Hz, 1H), 8.31 (s, 1H), 8.23 | | | | | | |
| | | (dd, J = 9.0, 1.8 Hz, 1H), 8.11 (s, | | | | | | |
| | | 1H), 7.87 (d, J = 8.8 Hz, 1H), 7.76- | | | | | | |
| | | 7.58 (m, 1H), 7.74 (d, J = 1.2 Hz, | | | | | | |
| | | 1H), 7.58 (dd, J = 14.2, 8.2 Hz, | | | | | | |
| | | 1H), 7.26 (td, J = 8.0, 1.7 Hz, 1H). | | | | | | |
| 214 | | ¹H-NMR (400 MHz, DMSO-d₆): δ | DMSO | 95 | R6 | 349.0 | t = 1.719 min | Method B |
| | | 8.65 (s, 3H), 8.35 (s, 1H), 8.20 (d, | | | | (M + 1) | | (NH4—HCO3) |
| | | J = 8.8 Hz, 1H), 7.84 (d, J = 8.4 | | | | | | |
| | | Hz, 1H), 7.74 (d, J = 8.4, Hz, 2H), | | | | | | |
| | | 7.62-7.56 (m, 1H), 7.52 (t, J = | | | | | | |
| | | 10.8 Hz, 1H), 7.27 (t, J = 16.4 Hz, | | | | | | |
| | | 1H), 3.13 (s, 3H). | | | | | | |
| 215 | | ¹H-NMR (400 MHz, DMSO-d₆): δ | DMSO | 96 | R6 | 361.1 | t = 1.674 min | Method B |
| | | 9.65 (d, J = 1.4 Hz, 1H), 8.79 (dt, J = | | | | (M + 1) | | (NH4—HCO3) |
| | | 8.0, 1.8 Hz, 1H), 8.69 (dd, J = | | | | | | |
| | | 4.7, 1.6 Hz, 1H), 8.56 (d, J = 4.3 | | | | | | |
| | | Hz, 1H), 8.31 (d, J = 8.6 Hz, 1H), | | | | | | |
| | | 7.90 (s, 1H), 7.68 (d, J = 8.5 Hz, | | | | | | |
| | | 1H), 7.55 (dd, J = 7.8, 4.8 Hz, 1H), | | | | | | |
| | | 7.32-7.19 (m, 3H), 3.91 (s, 3H), | | | | | | |
| | | 3.18 (d, J = 4.4 Hz, 3H). | | | | | | |
| 216 | | ¹H -NMR (400 MHz, MeOD): δ | MeOD | 95 | R6 | 331.0 | t = 1.701 min | Method B |
| | | 9.43 (s, 1H), 8.70 (d, J = 4.8 Hz, | | | | (M + 1) | | (NH4—HCO3) |
| | | 1H), 8.49(s, J1H), 8.14 (d, J = 5.8 | | | | | | |
| | | Hz, 1H), 7.93 (d, J = 4.0 Hz, 1H), | | | | | | |
| | | 7.44-7.66 (m, 3H), 7.16 (t, J = | | | | | | |
| | | 6.4 Hz, 1H), 2.64 (s, 3H). | | | | | | |
| 217 | | ¹H-NMR (400 MHz, DMSO-d₆): δ | DMSO | 95 | R6 | 332.7 | t = 1.751 min | Method B |
| | | 9.66 (s, 1H), 8.82 (s, 1H), 8.76 | | | | (M + 1) | | (NH4—HCO3) |
| | | (s,1 H), 8.70-8.67 (m, 2H), 8.23- | | | | | | |
| | | 8.21 (d, J = 10.4 Hz, 1H), 7.92- | | | | | | |
| | | 7.90 (d, J = 8.8 Hz, 1H), 7.75- | | | | | | |
| | | 7.73 (d, 2H), 7.62-7.56 (m, 1H), | | | | | | |
| | | 7.29-7.25 (t, J = 8.0 Hz, 1H), | | | | | | |
| | | 3.19-3.16 (d, J = 12.0 Hz, 3H). | | | | | | |
| 218 | | ¹H-NMR (400 MHz, DMSO-d₆): δ | DMSO | 98 | R6 | 361.1 | t = 1.706 min | Method B |
| | | 9.65 (s, 1H), 8.79 (d, J = 8.0 Hz, | | | | (M + 1) | | (NH4—HCO3) |
| | | 1H), 8.69 (d, J = 3.3 Hz, 1H), 8.52 | | | | | | |
| | | (d, J = 4.4 Hz, 1H), 8.29 (d, J = 8.6 | | | | | | |
| | | Hz, 1H), 7.89 (s, 1H), 7.68-7.64 | | | | | | |
| | | (m, 2H), 7.54 (dd, J = 7.7, 4.7 Hz, | | | | | | |
| | | 1H), 7.02 (dd, J = 13.2, 2.4 Hz, | | | | | | |
| | | 1H), 6.96 (dd, J = 8.7, 2.3 Hz, 1H), | | | | | | |
| | | 3.85 (s, 3H), 3.18 (d, J = 4.4 Hz, 3H). | | | | | | |
| 219 | | ¹H-NMR (400 MHz, DMSO-d₆): δ | DMSO | 96 | R6 | 361.1 | t = 1.703 min | Method B |
| | | 9.65 (d, J = 1.4 Hz, 1H), 8.79 (d, J = | | | | (M + 1) | | (NH4—HCO3) |
| | | 7.9 Hz, 1H), 8.69 (d, J = 3.4 Hz, | | | | | | |
| | | 1H), 8.57 (d, J = 4.5 Hz, 1H), 8.32 | | | | | | |
| | | (d, J = 8.6 Hz, 1H), 7.95 (s, 1H), | | | | | | |
| | | 7.72 (d, J = 8.6 Hz, 1H), 7.55 (dd, | | | | | | |
| | | J = 7.7, 4.7 Hz, 1H), 7.36-7.27 | | | | | | |
| | | (m, 1H), 7.22 (dd, J = 6.3, 3.1 Hz, | | | | | | |
| | | 1H), 7.08-6.98 (m, 1H), 3.84 (s, | | | | | | |
| | | 3H), 3.18 (d, J = 4.4 Hz, 3H). | | | | | | |
| 220 | | ¹H-NMR (400 MHz, DMSO-d₆): δ | DMSO | 93 | R6 | 345.1 | t = 1.688 min | Method B |
| | | 8.65 (d, J = 2.0 Hz, 1H), 8.61 (d, J = | | | | (M + 1) | | (NH4—HCO3) |
| | | 4.8 Hz, 1H), 8.53 (dd, J = 4.4, | | | | | | |
| | | 2.0 Hz, 1H), 8.27 (dd, J = 7.6, 1.2 | | | | | | |
| | | Hz, 1H), 8.20 (dd, J = 8.4, 2.0 Hz, | | | | | | |
| | | 1H), 7.83 (d, J = 8.8 Hz, 1H), 7.72 | | | | | | |
| | | (d, J = 8.0 Hz, 2H), 7.62-7.57 (m, | | | | | | |
| | | 1H), 7.36 (dd, J = 7.6, 4.8 Hz, 1H), | | | | | | |
| | | 7.29-7.25 (m, 1H), 3.12 (d, J = | | | | | | |
| | | 4.4 Hz, 3H), 2.81 (s, 3H). | | | | | | |
| 221 | | ¹H-NMR (400 MHz, DMSO-d₆): δ | DMSO | 95 | R6 | 367.0 | t = 1.799 min | Method B |
| | | 9.64 (s, 1H), 8.78 (d, J = 8 Hz, | | | | (M + 1) | | (NH4—HCO3) |
| | | 1H), 8.73-8.72 (m, 1H), 8.13- | | | | | | |
| | | 8.10(m, 1H), 7.87 (t, J = 16.4 Hz, | | | | | | |
| | | 1H), 7.76-7.73 (m, 1H), 7.59- | | | | | | |
| | | 7.56 (m, 2H), 7.42-7.39 (m, 2H), | | | | | | |
| | | 3.18 (s, 3H). | | | | | | |
| 222 | | ¹H-NMR (400 MHz, DMSO-d₆): δ | DMSO | 95 | R6 | 367.1 | t = 1.802 min | Method B |
| | | 9.63 (s, 1H), 8.77 (d, J = 8.0 Hz, | | | | (M + 1) | | (NH4—HCO3) |
| | | 1H), 8.72-8.71 (m, 1H), 8.09- | | | | | | |
| | | 8.07 (m, 1H), 7.83 (t, J = 16.8 Hz, | | | | | | |
| | | 1H), 7.73-7.71 (m, 1H), 7.69- | | | | | | |
| | | 7.63 (m, 1H), 7.58-7.55 (m, 1H), | | | | | | |
| | | 7.49 (t, J = 19.6 Hz, 1H), 7.29 (t, J = | | | | | | |
| | | 16.8 Hz, 1H), 3.18 (s, 3H). | | | | | | |
| 223 | | ¹H-NMR (400 MHz, DMSO-d₆): δ | DMSO | 95 | R6 | 349.1 | t = 1.813 min | Method B |
| | | 9.62 (s, 1H), 8.76-8.71 (m, 2H), | | | | (M + 1) | | (NH4—HCO3) |
| | | 8.06 (s, 1H), 7.92 (t, J = 16.8 Hz, | | | | | | |
| | | 1H), 7.70-7.68 (m, 1H), 7.57- | | | | | | |
| | | 7.50 (m, 4H), 7.31 (t, J = 15.2 Hz, | | | | | | |
| | | 1H), 3.18 (s, 3H). | | | | | | |
| 224 | | ¹H-NMR (400 MHz, DMSO-d₆): δ | DMSO | 95 | R1 | 401.5 | t = 1.191 min | Method A |
| | | 9.65 (d, J = 1.4 Hz, 1H), 8.79 (dt, J = | | | | (M + 1) | | (0.01% TFA) |
| | | 8.0, 1.8 Hz, 1H), 8.69 (dd, J = | | | | | | |
| | | 4.7, 1.6 Hz, 1H), 8.64 (d, J = 4.5 | | | | | | |
| | | Hz, 1H), 8.59 (d, J = 1.6 Hz, 1H), | | | | | | |
| | | 8.15 (dd, J = 8.7, 1.8 Hz, 1H), 7.85 | | | | | | |
| | | (d, J = 8.7 Hz, 1H), 7.55 (dd, J = | | | | | | |
| | | 7.6, 4.9 Hz, 1H), 7.47-7.38 (m, | | | | | | |
| | | 3H), 7.00 (dt, J = 7.3, 2.1 Hz, 1H), | | | | | | |
| | | 4.61 (d, J = 4.9 Hz, 1H), 4.22- | | | | | | |
| | | 4.09 (m, 2H), 3.93-3.82 (m, 1H), | | | | | | |
| | | 3.19 (d, J = 4.4 Hz, 3H), 1.90- | | | | | | |
| | | 1.72 (m, 2H), 1.16 (d, J = 6.2 Hz, 3H). | | | | | | |
| 225 | | ¹H-NMR (400 MHz, DMSO-d₆): δ | DMSO | 100 | R1 | 401.47 | t = 1.196 min | Method A |
| | | 9.65 (d, J = 1.4 Hz, 1H), 8.79 (dt, J = | | | | (M + 1) | | (0.01% TFA) |
| | | 8.0, 1.8 Hz, 1H), 8.69 (dd, J = | | | | | | |
| | | 4.7, 1.6 Hz, 1H), 8.64 (d, J = 4.5 | | | | | | |
| | | Hz, 1H), 8.59 (d, J = 1.6 Hz, 1H), | | | | | | |
| | | 8.15 (dd, J = 8.7, 1.8 Hz, 1H), 7.85 | | | | | | |
| | | (d, J = 8.7 Hz, 1H), 7.55 (dd, J = | | | | | | |
| | | 7.6, 4.9 Hz, 1H), 7.47-7.38 (m, | | | | | | |
| | | 3H), 7.00 (dt, J = 7.3, 2.1 Hz, 1H), | | | | | | |
| | | 4.61 (d, J = 4.9 Hz, 1H), 4.22- | | | | | | |
| | | 4.09 (m, 2H), 3.93-3.82 (m, 1H), | | | | | | |
| | | 3.19 (d, J = 4.4 Hz, 3H), 1.83-1.81 | | | | | | |
| | | (m, 2H), 1.16 (d, J = 6.2 Hz, 3H). | | | | | | |
| 226 | | ¹H-NMR (400 MHz, DMSO-d₆): δ | DMSO | 96 | R1 | 415.2 | t = 1.342 min | Method A |
| | | 9.66 (s, 1H), 8.79 (dt, J = 8.0, 1.8 | | | | (M + 1) | | (0.01% TFA) |
| | | Hz, 1H), 8.69 (dd, J = 4.6, 1.2 Hz, | | | | | | |
| | | 1H), 8.65 (d, J = 4.5 Hz, 1H), 8.59 | | | | | | |
| | | (d, J = 1.4 Hz, 1H), 8.16 (dd, J = | | | | | | |
| | | 8.7, 1.6 Hz, 1H), 7.85 (d, J = 8.7 | | | | | | |
| | | Hz, 1H), 7.55 (dd, J = 7.8, 4.8 Hz, | | | | | | |
| | | 1H), 7.49-7.39 (m, 3H), 7.00 (dt, | | | | | | |
| | | J = 4.9, 2.2 Hz, 1H), 4.14 (h, J = | | | | | | |
| | | 9.5 Hz, 2H), 3.54 (tt, J = 9.5 4.6 Hz, | | | | | | |
| | | 1H), 3.35 (s, 1H), 3.25 (s, 3H), 3.19 | | | | | | |
| | | (d, J = 4.4 Hz, 3H), 1.98-1.81 (m, 2H), | | | | | | |
| | | 1.17 (d, J = 6.1 Hz, 3H). | | | | | | |
| 227 | | ¹H-NMR (400 MHz, DMSO-d₆): δ | DMSO | 95 | R1 | 415.1 | t = 1.341 min | Method A |
| | | 9.65 (s, 1H), 8.79 (d, J = 8.0 Hz, | | | | (M + 1) | | (CF3—COOH) |
| | | 1H), 8.69 (d, J = 3.8 Hz, 1H), 8.65 | | | | | | |
| | | (d, J = 4.5 Hz, 1H), 8.59 (d, J = 1.4 | | | | | | |
| | | Hz, 1H), 8.16 (dd, J = 8.7, 1.6 Hz, | | | | | | |
| | | 1H), 7.85 (d, J = 8.7 Hz, 1H), 7.55 | | | | | | |
| | | (dd, J = 7.9, 4.8 Hz, 1H), 7.48- | | | | | | |
| | | 7.38 (m, 3H), 7.00 (dt, J = 4.9, 2.3 | | | | | | |
| | | Hz, 1H), 4.20-4.09 (m, 2H), 3.56 | | | | | | |
| | | (dt, J = 12.3, 6.1 Hz, 1H), 3.35 (s, | | | | | | |
| | | 1H), 3.25 (s, 3H), 3.19 (d, J = 4.4 | | | | | | |
| | | Hz, 3H), 1.91-1.89 (m, 2H), 1.17 | | | | | | |
| | | (d, J = 6.1 Hz, 3H). | | | | | | |
| 228 | 2HCl | ¹H-NMR (400 MHz, DMSO-d₆): δ | DMSO | 95 | R1 | 402.1 | t = 1.544 min | Method B |
| | | 10.34 (s, 1H), 9.71 (s, 1H), 9.16 (d, | | | | (M + 1) | | (NH4—HCO3) |
| | | J = 7.8 Hz, 1H), 8.99 (d, J = 5.0 | | | | | | |
| | | Hz, 1H), 8.82 (s, 1H), 8.33 (d, J = | | | | | | |
| | | 8.7 Hz, 1H), 8.23 (d, J = 8.6 Hz, | | | | | | |
| | | 1H), 7.94 (dd, J = 7.8, 5.2 Hz, 1H), | | | | | | |
| | | 7.80 (t, J = 7.9 Hz, 1H), 7.50 (dd, J = | | | | | | |
| | | 11.1, 1.2 Hz, 1H), 7.44 (dd, J = | | | | | | |
| | | 7.9, 1.4 Hz, 1H), 3.30 (d, J = 4.4 | | | | | | |
| | | Hz, 3H), 3.03 (s, 3H), 2.98 (s, 3H). | | | | | | |
| 229 | 2HCl | ¹H-NMR (400 MHz, DMSO-d₆): δ | DMSO | 95 | R1 | 361.1 | t = 1.87 min | Method B |
| | | 10.44 (s, 1H), 9.68 (s, 1H), 9.11 (s, | | | | (M + 1) | | (NH4—HCO3) |
| | | 1H), 8.93 (d, J = 27.2 Hz, 2H), | | | | | | |
| | | 8.32 (d, J = 25.7 Hz, 2H), 7.90 (s, | | | | | | |
| | | 1H), 7.74 (d, J = 13.4 Hz, 2H), | | | | | | |
| | | 7.33 (s, 1H), 3.31 (s, 3H), 2.42 (s, 3H). | | | | | | |
| 230 | 2HCl | ¹H-NMR (400 MHz, DMSO-d₆): δ | DMSO | 95 | R1 | 379.0 | t = 1.68 min | Method |
| | | 10.03 (s, 1H), 9.70 (s, 1H), 9.11 (d, | | | | (M + 1) | | (NH4—HCO3) |
| | | J = 7.3 Hz, 1H), 8.99 (d, J = 4.8 | | | | | | |
| | | Hz, 1H), 8.54 (s, 1H), 7.92 (dd, J = | | | | | | |
| | | 7.6, 5.3 Hz, 1H), 7.87 (s, 1H), 7.57 | | | | | | |
| | | (dd, J = 16.7, 8.2 Hz, 1H), 7.37- | | | | | | |
| | | 7.35 (m, 2H), 3.96 (s, 3H), 3.26 (d, | | | | | | |
| | | J = 4.3 Hz, 3H). | | | | | | |
| 231 | 2HCl | ¹H-NMR (400 MHz, DMSO-d₆): δ | DMSO | 98 | R1 | 355.1 | tt = 1.706 min | Method B |
| | | 10.39 (s, 1H), 9.67 (s, 1H), 9.05 (d, | | | | (M + 1) | | (NH4—HCO3) |
| | | J = 7.9 Hz, 1H), 8.95 (d, J = 4.3 | | | | | | |
| | | Hz, 1H), 8.79 (s, 1H), 8.28 (dd, J = | | | | | | |
| | | 25.1, 8.6 Hz, 2H), 7.91-7.83 (m, | | | | | | |
| | | 1H), 7.80 (s, 1H), 7.66 (d, J = 8.1 | | | | | | |
| | | Hz, 1H), 6.92 (d, J = 8.2 Hz, 1H), | | | | | | |
| | | 4.62 (t, J = 8.5 Hz, 2H), 3.31 (d, J = | | | | | | |
| | | 3.9 Hz, 3H), 3.27 (d, J = 8.4 Hz, 2H). | | | | | | |
| 232 | 3HCl | ¹H-NMR (400 MHz, DMSO-d₆): δ | DMSO | 95 | R1 | 371.1 | t = 1.363 min | Method B |
| | | (s, 1H), 9.18 (d, J = 1.7 Hz, | | | | (M + 1) | | (NH4—HCO3) |
| | | 1H), 9.05 (s, 2H), 8.95 (d, J = 4.5 | | | | | | |
| | | Hz, 1H), 8.87 (d, J = 4.8 Hz, 1H), | | | | | | |
| | | 8.51-8.43 (m, 2H), 8.18 (d, J = | | | | | | |
| | | 8.1 Hz, 2H), 7.93-7.84 (m, 1H), | | | | | | |
| | | 3.31 (d, J = 4.2 Hz, 3H), 2.86 (d, J = | | | | | | |
| | | 4.7 Hz, 3H). | | | | | | |
| 233 | | ¹H-NMR (400 MHz, DMSO-d₆): δ | DMSO | 100 | R1 | 371.0 | t = 1.745 min | Method B |
| | | 9.66 (s, 1H), 8.80 (d, J = 8.0 Hz, | | | | (M + 1) | | (NH4—HCO3) |
| | | 1H), 8.70 (d, J = 3.5 Hz, 1H), 8.68 | | | | | | |
| | | (s, 1H), 8.65 (d, J = 4.4 Hz, 1H), | | | | | | |
| | | 8.29 (dd, J = 8.8, 1.4 Hz, 1H), 8.22 | | | | | | |
| | | (d, J = 1.9 Hz, 1H), 7.93 (d, J = 8.7 | | | | | | |
| | | Hz, 1H), 7.70 (dd, J = 8.3, 5.4 Hz, | | | | | | |
| | | 1H), 7.56 (dd, J = 7.8, 4.6 Hz, 1H), | | | | | | |
| | | 7.32 (t, J = 8.9 Hz, 1 H), 7.22 (d, J = | | | | | | |
| | | 2.0 Hz, 1H), 3.19 (d, J = 4.2 Hz, 3H). | | | | | | |
| 234 | 2HCl | ¹H-NMR (400 MHz, MeOD): δ | MeOD | 95 | R1 | 367.0 | t = 1.620 min | Method A |
| | | 9.73 (s, 1H), 9.32 (d, J = 8.2 Hz, | | | | (M + 1) | | (TFA) |
| | | 1H), 9.05 (d, J = 5.3 Hz, 1H), 8.24 | | | | | | |
| | | (s, 1H), 8.19 (dd, J = 7.9, 5.8 Hz, | | | | | | |
| | | 1H), 8.04 (d, J = 8.6 Hz, 1H), 7.99- | | | | | | |
| | | 7.94 (m, 1H), 7.16-7.07 (m, | | | | | | |
| | | 2H), 7.01 (d, J = 7.0 Hz, 1H), 3.36 | | | | | | |
| | | (s, 3H), 2.80 (t, J = 6.2 Hz, 2H), | | | | | | |
| | | 2.56 (t, J = 6.1 Hz, 2H), 1.77- | | | | | | |
| | | 1.75 (m, 2H), 1.71-1.59 (m, 2H). | | | | | | |
| 235 | 2HCl | ¹H-NMR (400 MHz, DMSO-d₆): δ | DMSO | 95 | R1 | 392.0 | t = 1.45 min | Method B |
| | | 10.21 (s, 1H), 9.65 (s, 1H), 9.04 (d, | | | | (M + 1) | | (NH4—HCO3) |
| | | J = 8.2 Hz, 1H), 8.99 (s, 1H), 8.96 | | | | | | |
| | | (d, J = 4.4 Hz, 1H), 8.39 (s, 1H), | | | | | | |
| | | 8.37 (s, 1H), 8.20 (d, J = 8.6 Hz, | | | | | | |
| | | 1H), 8.13 (d, J = 7.9 Hz, 1H), 7.93- | | | | | | |
| | | 7.86 (m, 2H), 7.78 (t, J = 7.8 Hz, | | | | | | |
| | | 1H), 7.52 (s, 2H), 3.32 (d, J = 4.3 | | | | | | |
| | | Hz, 3H). | | | | | | |
| 236 | | ¹H-NMR (400 MHz, DMSO-d₆): δ | DMSO | 99 | R1 | 396.1 | t = 1.488 min | Method B |
| | | 9.66 (s, 1H), 8.79 (d, J = 7.7 Hz, | | | | (M + 1) | | (NH4—HCO3) |
| | | 1H), 8.73-8.63 (m, 2H), 8.59 (s, | | | | | | |
| | | 1H), 8.12 (d, J = 8.8 Hz, 1H), 8.02 | | | | | | |
| | | (s, 1H), 7.88 (d, J = 8.6 Hz, 1H), | | | | | | |
| | | 7.75 (d, J = 7.8 Hz, 1H), 7.62 (d, J = | | | | | | |
| | | 7.6 Hz, 1H), 7.54 (t, J = 7.7 Hz, | | | | | | |
| | | 2H), 3.98 (t, J = 6.9 Hz, 2H), 3.19 | | | | | | |
| | | (d, J = 4.1 Hz, 3H), 2.61-2.53 (m, | | | | | | |
| | | 2H), 2.17-2.05 (m, 2H). | | | | | | |
| 237 | 2HCl | ¹H-NMR (400 MHz, DMSO-d₆): δ | DMSO | 95 | R1 | 344.9 | t = 1.453 min | Method A |
| | | 10.11 (s, 1H), 9.66 (s, 1H), 9.05 (d, | | | | (M + 1) | | (TFA) |
| | | J = 7.1 Hz, 1H), 8.96 (d, J = 4.6 | | | | | | |
| | | Hz, 1H), 8.69 (s, 1H), 8.20 (s, 1H), | | | | | | |
| | | 7.89 (dd, J = 7.1, 4.8 Hz, 1H), 7.51 | | | | | | |
| | | (t, J = 7.3 Hz, 1H), 7.41 (t, J = 7.2 | | | | | | |
| | | Hz, 1H), 7.29 (t, J = 7.6 Hz, 1H), | | | | | | |
| | | 3.30 (d, J = 4.3 Hz, 3H), 2.35 (s, 3H). | | | | | | |
| 238 | 2HCl | ¹H-NMR (400 MHz, DMSO-d₆): δ | DMSO | 95 | R1 | 411.0 | t = 1.91 min | Method B |
| | | 9.63 (s, 2H), 8.95 (d, J = 7.8 Hz, | | | | (M + 1) | | (NH4—HCO3) |
| | | 1H), 8.89 (d, J = 3.9 Hz, 1H), 8.79 | | | | | | |
| | | (s, 1H), 8.33 (d, J = 8.4 Hz, 1H), | | | | | | |
| | | 8.06 (d, J = 8.6 Hz, 1H), 7.80 (d, J = | | | | | | |
| | | 8.6 Hz, 2H), 7.67 (s, 1H), 7.30 | | | | | | |
| | | (s, 1H), 3.29 (d, J = 4.3 Hz, 3H), | | | | | | |
| | | 2.49 (s, 3H). | | | | | | |
| 239 | 2HCl | ¹H-NMR (400 MHz, DMSO-d₆): δ | DMSO | 97 | R1 | 344.9 | t = 1.710 min | Method B |
| | | 10.42 (s, 1H), 9.71 (d, J = 1.6 Hz, | | | | (M + 1) | | (NH4—HCO3) |
| | | 1H), 9.15 (d, J = 8.0 Hz, 1H), 8.99 | | | | | | |
| | | (d, J = 8.0 Hz, 1H), 8.76 (s, 1H), | | | | | | |
| | | 8.32 (d, J = 8.6 Hz, 1H), 8.18 (d, J = | | | | | | |
| | | 8.7 Hz, 1H), 7.92 (dd, J = 7.9, 5.2 | | | | | | |
| | | Hz, 1H), 7.62 (d, J = 8.0 Hz, 1H), | | | | | | |
| | | 7.22 (t, J = 10.3 Hz, 2H), 3.30 (d, J = | | | | | | |
| | | 4.4 Hz, 3H), 2.40 (s, 3H). | | | | | | |
| 240 | 2HCl | ¹H-NMR (400 MHz, DMSO-d₆): δ | DMSO | 95 | R1 | 365.0 | t = 1.76 min | Method B |
| | | 10.50 (s, 1H), 9.76 (s, 1H), 9.26 (d, | | | | (M + 1) | | (NH4—HCO3) |
| | | J = 8.1 Hz, 1H), 9.03 (d, J = 4.5 | | | | | | |
| | | Hz, 1H), 8.83 (s, 1H), 8.37 (d, J = | | | | | | |
| | | 8.7 Hz, 1H), 8.22 (d, J = 8.7 Hz, | | | | | | |
| | | 1H), 8.00 (dd, J = 7.9, 5.3 Hz, 1H), | | | | | | |
| | | 7.82 (dd, J = 6.7, 2.5 Hz, 1H), 7.62- | | | | | | |
| | | 7.53 (m, 1H), 7.52-7.43 (m, | | | | | | |
| | | 1H), 3.31 (d, J = 4.3 Hz, 3H). | | | | | | |
| 241 | | ¹H-NMR (400 MHz, DMSO-d₆): δ | DMSO | 95 | R1 | 365.1 | t = 1.752 min | Method B |
| | | 9.65 (d, J = 1.4 Hz, 1H), 8.79 (dt, J = | | | | (M + 1) | | (NH4—HCO3) |
| | | 8.0, 1.9 Hz, 1H), 8.70 (dd, J = | | | | | | |
| | | 4.7, 1.7 Hz, 1H), 8.53 (d, J = 4.4 | | | | | | |
| | | Hz, 1H), 8.31 (s, 1H), 7.89 (d, J = | | | | | | |
| | | 8.5 Hz, 1H), 7.78 (dd, J = 8.6, 1.3 | | | | | | |
| | | Hz, 1H), 7.59-7.52 (m, 3H), 7.47- | | | | | | |
| | | 7.39 (m, 1H), 3.15 (d, J = 4.5 Hz, 3H). | | | | | | |
| 242 | 2HCl | ¹H-NMR (400 MHz, DMSO-d₆): δ | DMSO | 95 | R1 | 375.0 | t = 1.716 min | Method B |
| | | 9.64 (s, 1H), 9.02 (d, J = 7.8 Hz, | | | | (M + 1) | | (NH4—HCO3) |
| | | 1H), 8.95 (d, J = 3.9 Hz, 1H), 8.67 | | | | | | |
| | | (s, 1H), 8.17 (s, 2H), 7.92-7.82 | | | | | | |
| | | (m, 1H), 7.35-7.17 (m, 3H), 4.19 | | | | | | |
| | | (q, J = 7.0 Hz, 2H), 3.29 (d, J = 4.4 | | | | | | |
| | | Hz, 3H), 1.39 (t, J = 7.0 Hz, 3H). | | | | | | |
| 243 | 2HCl | ¹H NMR (400 MHz, DMSO-d₆): δ | DMSO | 95 | R1 | 382.7 | t = 1.621 min | Method A |
| | | 9.64 (s, 1H), 9.42 (d, J = 8.2 Hz, | | | | (M + 1) | | (TFA) |
| | | 1H), 9.20 (d, J = 4.4 Hz, 1H), 9.07 | | | | | | |
| | | (d, J = 5.3 Hz, 1H), 8.56 (s, 1H), | | | | | | |
| | | 8.22-8.20 (m, 2H), 7.61-7.49 | | | | | | |
| | | (m, 2H), 7.40-7.38 (m, 1H), 3.17 | | | | | | |
| | | (d, J = 4.2 Hz, 3H). | | | | | | |
| 244 | 2HCl | ¹H-NMR (400 MHz, DMSO-d₆): δ | DMSO | 95 | R1 | 383.0 | t = 2.009 min | Method B |
| | | 9.69 (s, 1H), 9.11 (d, J = 8.0 Hz, | | | | (M + 1) | | (NH4—HCO3) |
| | | 1H), 8.93 (d, J = 4.5 Hz, 1H), 8.88 | | | | | | |
| | | (d, J = 4.4 Hz, 1H), 8.44 (s, 1H), | | | | | | |
| | | 8.15 (s, 1H), 7.88 (dd, J = 7.9, 5.2 | | | | | | |
| | | Hz, 1H), 7.77 (td, J = 8.9, 6.7 Hz, | | | | | | |
| | | 1H), 7.53-7.45 (m, 1H), 7.31 (td, | | | | | | |
| | | J = 8.4, 2.2 Hz, 1H), 3.19 (d, J = | | | | | | |
| | | 4.4 Hz, 3H). | | | | | | |
| 245 | 2HCl | ¹H-NMR (400 MHz, DMSO-d₆): δ | DMSO | 95 | R1 | 390.9 | t = 1.507 min | Method B |
| | | 10.66 (s, 1H), 9.70 (s, 1H), 9.13 (d, | | | | (M + 1) | | (NH4—HCO3) |
| | | J = 11.1 Hz, 2H), 8.98 (d, J = 4.6 | | | | | | |
| | | Hz, 1H), 8.46 (d, J = 12.8 Hz, 2H), | | | | | | |
| | | 8.32 (d, J = 8.6 Hz, 1H), 8.28 (d, J = | | | | | | |
| | | 7.6 Hz, 1H), 8.01 (d, J = 7.5 Hz, | | | | | | |
| | | 1H), 7.93 (d, J = 5.2 Hz, 1H), 7.84 | | | | | | |
| | | (t, J = 7.8 Hz, 1H), 3.40 (s, 3H), | | | | | | |
| | | 3.32 (d, J = 3.9 Hz, 3H). | | | | | | |
| 246 | 2HCl | ¹H-NMR (400 MHz, DMSO-d₆): δ | DMSO | 98 | R1 | 383.0 | t = 1.296 min | Method A |
| | | 9.80 (s, 1H), 9.71 (s, 1H), 9.30 (d, | | | | (M + 1) | | (TFA) |
| | | J = 8.2 Hz, 1H), 9.03 (d, J = 4.2 | | | | | | |
| | | Hz, 1H), 8.62 (s, 1H), 8.33 (s, 1H), | | | | | | |
| | | 8.06 (dd, J = 7.8, 5.5 Hz, 2H), 7.60 | | | | | | |
| | | (dd, J = 15.2, 8.6 Hz, 1H), 7.50 (td, | | | | | | |
| | | J = 9.8, 2.4 Hz, 1H), 7.32 (td, J = | | | | | | |
| | | 8.5, 2.1 Hz, 1H), 3.22 (d, J = 4.4 | | | | | | |
| | | Hz, 3H). | | | | | | |
| 247 | 2HCl | ¹H-NMR (400 MHz, MeOD): δ 9.62 | CD₃OD | 95 | R1 | 345.1 | t = 1.912 min | Method B |
| | | (d, J = 1.4 Hz, 1H), 8.89 (dt, J = | | | | (M + 1) | | (NH4—HCO3) |
| | | 8.0, 1.9 Hz, 1H), 8.65 (dd, J = 4.9, | | | | | | |
| | | 1.5 Hz, 1H), 8.04 (d, J = 1.6 Hz, | | | | | | |
| | | 1H), 7.90 (d, J = 8.5 Hz, 1H), 7.75 | | | | | | |
| | | (dd, J = 8.6, 1.9 Hz, 1H), 7.60 (dd, | | | | | | |
| | | J = 7.7, 4.7 Hz, 1H), 7.33 (dd, J = | | | | | | |
| | | 8.4, 5.9 Hz, 1H), 7.10 (dd, J = 9.9, | | | | | | |
| | | 2.5 Hz, 1H), 7.04 (td, J = 8.5, 2.6 | | | | | | |
| | | Hz, 1H), 3.26 (s, 3H), 2.33 (s, 3H). | | | | | | |
| 248 | 2HCl | ¹H-NMR (400 MHz, DMSO-d₆): δ | DMSO | 95 | R1 | 399.0 | t = 1.275 min | Method A |
| | | 10.21 (s, 1H), 9.71 (d, J = 1.6 Hz, | | | | (M + 1) | | (TFA) |
| | | 1H), 9.17 (d, J = 8.0 Hz, 1H), 9.00 | | | | | | |
| | | (d, J = 4.1 Hz, 1H), 8.55 (s, 1H), | | | | | | |
| | | 8.31 (d, J = 8.5 Hz, 1H), 7.95 (t, J = | | | | | | |
| | | 8.0 Hz, 2H), 7.87 (dd, J = 9.3, | | | | | | |
| | | 2.5 Hz, 1H), 7.74 (td, J = 8.4, 2.4 | | | | | | |
| | | Hz, 1H), 7.63 (dd, J = 8.4, 5.6 Hz, | | | | | | |
| | | 1H), 3.27 (d, J = 4.4 Hz, 3H). | | | | | | |
| 249 | 2HCl | ¹H-NMR (400 MHz, DMSO-d₆): δ | DMSO | 97 | R1 | 411.1 | t = 1.963 min | Method B |
| | | 10.04 (s, 1H), 9.67 (d, J = 1.6 Hz, | | | | (M + 1) | | (NH4—HCO3) |
| | | 1H), 9.06 (d, J = 8.0 Hz, 1H), 8.94 | | | | | | |
| | | (d, J = 3.9 Hz, 1H), 8.49 (s, 1H), | | | | | | |
| | | 8.24 (d, J = 8.4 Hz, 1H), 7.91 (d, J = | | | | | | |
| | | 8.6 Hz, 1H), 7.85 (dd, J = 7.8, | | | | | | |
| | | 5.2 Hz, 1H), 7.48 (d, J = 8.0 Hz, | | | | | | |
| | | 1H), 7.38 (d, J = 7.9 Hz, 2H), 3.92 | | | | | | |
| | | (s, 3H), 3.26 (d, J = 4.4 Hz, 3H). | | | | | | |
| 250 | 2HCl | ¹H-NMR (400 MHz, DMSO-d₆): δ | DMSO | 95 | R1 | 380.0 | t = 1.66 min | Method B |
| | | 10.44 (s, 1H), 9.71 (s, 1H), 9.14 (d, | | | | (M + 1) | | (NH4—HCO3) |
| | | J = 8.0 Hz, 1H), 8.98 (d, J = 6.8 | | | | | | |
| | | Hz, 2H), 8.45 (t, J = 3.4 Hz, 2H), | | | | | | |
| | | 8.34 (d, J = 9.0 Hz, 2H), 8.07 (dd, | | | | | | |
| | | J = 7.7, 1.3 Hz, 2H), 7.96-7.88 | | | | | | |
| | | (m, 1H), 7.73 (t, J = 7.8 Hz, 1H), | | | | | | |
| | | 7.48 (s, 1H), 3.33 (d, J = 4.4 Hz, 3H). | | | | | | |
| 251 | 2HCl | ¹H-NMR (400 MHz, DMSO-d₆): δ | DMSO | 100 | R1 | 379.1 | t = 1.721 min | Method B |
| | | 10.79 (s, 1H), 9.75 (s, 1H), 9.19 (d, | | | | (M + 1) | | (NH4—HCO3) |
| | | J = 8.0 Hz, 1H), 9.15 (s, 1H), 9.00 | | | | | | |
| | | (d, J = 4.7 Hz, 1H), 8.48 (d, J = 8.7 | | | | | | |
| | | Hz, 1H), 8.44 (s, 1H), 8.39 (d, J = | | | | | | |
| | | 8.7 Hz, 1H), 7.99-7.87 (m, 3H), | | | | | | |
| | | 7.85 (d, J = 1.9 Hz, 1H), 7.60 (t, J = | | | | | | |
| | | 7.7 Hz, 1H), 7.05 (d, J = 1.9 Hz, | | | | | | |
| | | 1H), 3.33 (d, J = 4.2 Hz, 3H). | | | | | | |
| 252 | 2HCl | ¹H-NMR (400 MHz, MeOD): δ 9.67 | MeOD | 95 | R1 | 379.0 | t = 1.666 min | Method B |
| | | (s, 1H), 9.31-9.23 (m, 1H), 9.13 | | | | (M + 1) | | (NH4—HCO3) |
| | | (t, J = 4.9 Hz, 1H), 8.26 (dd, J = | | | | | | |
| | | 14.3, 8.3 Hz, 1H), 8.13 (s, 1H), | | | | | | |
| | | 7.82 (s, 1H), 7.52 (ddd, J = 9.0, | | | | | | |
| | | 6.0, 3.1 Hz, 1H), 7.37 (td, J = 9.5, | | | | | | |
| | | 4.5 Hz, 1H), 7.32-7.25 (m, 1H), | | | | | | |
| | | 4.24 (s, 3H), 3.45 (s, 3H). | | | | | | |
| 253 | 2HCl | ¹H-NMR (400 MHz, MeOD): δ 9.66 | MeOD | 95 | R1 | 361.0 | t = 1.665 min | Method B |
| | | (d, J = 1.2 Hz, 1H), 9.25 (d, J = 8.3 | | | | (M + 1) | | (NH4—HCO3) |
| | | Hz, 1H), 9.13 (d, J = 5.5 Hz, 1H), | | | | | | |
| | | 8.24 (dd, J = 7.9, 3.6 Hz, 2H), 7.90 | | | | | | |
| | | (d, J = 1.2 Hz, 1H), 7.72 (d, J = 7.8 | | | | | | |
| | | Hz, 1H), 7.69-7.64 (m, 1H), 7.60 | | | | | | |
| | | (td, J = 8.0, 6.2 Hz, 1H), 7.26 (td, J = | | | | | | |
| | | 8.4, 1.9 Hz, 1H), 4.28 (s, 3H), 3.47 | | | | | | |
| | | (s, 3H). | | | | | | |
| 254 | 2HCl | ¹H-NMR (400 MHz, DMSO-d₆) δ | DMSO | 95 | R1 | 361.1 | t = 1.833 min | Method B |
| | | 9.58 (s, 1H), 9.31 (s, 1H) 9.25 (d, | | | | (M + 1) | | (NH4—HCO3) |
| | | J = 7.8 Hz, 1H), 9.02 (d, J = 5.4 | | | | | | |
| | | Hz, 1H), 8.16 (s, 1H), 8.11 (dd, J = | | | | | | |
| | | 7.9, 5.6 Hz, 1H), 7.74 (dd, J = 8.7, | | | | | | |
| | | 7.2 Hz, 1H), 7.56 (s, 1H), 7.55- | | | | | | |
| | | 7.48 (m, 1H), 7.40 (dd, J = 13.2, | | | | | | |
| | | 6.7 Hz, 2H), 4.07 (s, 3H), 3.20 (d, | | | | | | |
| | | J = 3.8 Hz, 3H). | | | | | | |
| 255 | 2HCl | ¹H-NMR (400 MHz, MeOD): δ9.66 | MeOD | 95 | R1 | 379.0 | t = 1.686 min | Method B |
| | | (d, J = 1.5 Hz, 1H), 9.25 (d, J = 8.2 | | | | (M + 1) | | (NH4—HCO3) |
| | | Hz, 1H), 9.12 (d, J = 4.6 Hz, 1H), | | | | | | |
| | | 8.24 (dd, J = 8.1, 5.7 Hz, 1H), 8.21 | | | | | | |
| | | (d, J = 1.4 Hz, 1H), 7.90-7.82 (m, | | | | | | |
| | | 2H), 7.75-7.69 (m, 1H), 7.53- | | | | | | |
| | | 7.44 (m, 1H), 4.28 (s, 3H), 3.46 (s, 3H). | | | | | | |
| 256 | 2HCl | ¹H NMR (400 MHz, MeOD): δ 9.74 | MeOD | 95 | R1 | 419.1 | t = 1.633 min | Method B |
| | | (s, 1H), 9.40 (d, J = 8.1 Hz, 1H), | | | | (M + 1) | | (NH4—HCO3) |
| | | 9.08 (d, J = 5.4 Hz, 1H), 8.75 (s, | | | | | | |
| | | 1H), 8.30-8.23 (m, 2H), 8.04 (d, J = | | | | | | |
| | | 8.7 Hz, 1H), 7.87 (dd, J = 8.3, | | | | | | |
| | | 3.1 Hz, 2H), 7.76 (t, J = 6.5 Hz, | | | | | | |
| | | 2H), 3.41 (s, 3H), 3.19-3.12 (m, | | | | | | |
| | | 1H), 1.11 (dd, J = 6.8, 1.8 Hz, 6H). | | | | | | |
| 257 | 2HCl | ¹H-NMR (400 MHz, DMSO-d₆): δ | DMSO | 97 | R1 | 370.9 | t = 1.442 min | Method A |
| | | 10.52 (s, 1H), 9.70 (s, 1H), 9.11 (d, | | | | (M + 1) | | (TFA) |
| | | J = 5.8 Hz, 1H), 8.97 (d, J = 4.7 | | | | | | |
| | | Hz, 1H), 8.93 (s, 1H), 8.39 (d, J = | | | | | | |
| | | 8.6 Hz, 1H), 8.33 (s, 1H), 7.96- | | | | | | |
| | | 7.83 (m, 1H), 7.45 (s, 3H), 7.08- | | | | | | |
| | | 6.98 (m, 1H), 4.83 (dt, J = 11.9, | | | | | | |
| | | 5.9 Hz, 1H), 1.32 (d, J = 5.9 Hz, 6H). | | | | | | |
| 258 | 2HCl | ¹H-NMR (400 MHz, DMSO-d₆): δ | DMSO | 93 | R1 | 395.1 | t = 1.508 min | Method A |
| | | 9.62 (s, 1H), 9.14 (s, 1H), 8.83 (s, | | | | (M + 1) | | (TFA) |
| | | 1H), 8.78 (s, 1H), 8.65 (s, 1H), | | | | | | |
| | | 8.19 (s, 1H), 7.94 (s, 1H), 7.78 (d, | | | | | | |
| | | J = 8.0 Hz, 2H), 7.65 (s, 1H), 7.39 | | | | | | |
| | | (d, J = 8.0 Hz, 2H), 3.23 (s, 3H), | | | | | | |
| | | 2.58 (s, 1H), 1.82 (s, 4H), 1.73 (d, | | | | | | |
| | | J = 11.6 Hz, 1H), 1.56-1.17 (m, 5H). | | | | | | |
| 259 | | ¹H-NMR (400 MHz, DMSO-d₆): δ | DMSO | 93 | R1 | 361.1 | t = 1.636 min | Method B |
| | | 9.63 (s, 1H), 8.77 (d, J = 7.8 Hz, | | | | (M + 1) | | (NH4—HCO3) |
| | | 1H), 8.68 (d, J = 3.6 Hz, 1H), 8.51 | | | | | | |
| | | (d, J = 3.8 Hz, 1H), 8.11 (s, 1H), | | | | | | |
| | | 7.94 (dd, J = 7.7, 5.7 Hz, 2H), 7.54 | | | | | | |
| | | (s, 2H), 7.39 (t, J = 8.6 Hz, 2H), | | | | | | |
| | | 4.08 (s, 3H), 3.17 (d, J = 3.8 Hz, 3H). | | | | | | |
| 260 | | ¹H-NMR (400 MHz, DMSO-d₆): δ | DMSO | 98 | R1 | 368.0 | t = 1.576 min | Method B |
| | | 9.64 (s, 1H), 8.78-8.57 (m, 3H), | | | | (M + 1) | | (NH4—HCO3) |
| | | 8.23-8.03 (m, 5H), 7.62-7.53 | | | | | | |
| | | (m, 2H), 4.09 (s, 3H), 3.18 (s, 3H). | | | | | | |
| 261 | 2HCl | ¹H-NMR (400 MHz, DMSO-d₆): δ | DMSO | 95 | R1 | 352.0 | t = 1.675 min | Method B |
| | | 11.49 (s, 1H), 10.35 (s, 1H), 9.72 | | | | (M + 1) | | (NH4—HCO3) |
| | | (s, 1H), 9.12 (d, J = 7.6 Hz, 1H), | | | | | | |
| | | 8.99 (d, J = 4.1 Hz, 1H), 8.79 (s, | | | | | | |
| | | 1H), 8.38 (s, 2H), 7.99-7.86 (m, | | | | | | |
| | | 1H), 7.52 (d, J = 6.7 Hz, 2H), 7.38- | | | | | | |
| | | 7.23 (m, 2H), 6.65 (s, 1H), 3.31 | | | | | | |
| | | (d, J = 4.0 Hz, 3H). | | | | | | |
| 262 | | ¹H-NMR (400 MHz, DMSO-d₆): δ | DMSO | 95 | R1 | 381.1 | t = 1.280 min | Method B |
| | | 9.66 (s, 1H), 8.80 (d, J = 7.9 Hz, | | | | (M + 1) | | (NH4—HCO3) |
| | | 1H), 8.70-8.68 (m, 3H), 8.24 (d, J = | | | | | | |
| | | 9.8 Hz, 1H), 8.21 (d, J = 8.2 Hz, | | | | | | |
| | | 2H), 8.11 (d, J = 8.3 Hz, 2H), 7.90 | | | | | | |
| | | (d, J = 8.7 Hz, 1H), 7.56 (dd, J = | | | | | | |
| | | 7.5, 4.9 Hz, 1H), 3.21 (d, J = 4.2 | | | | | | |
| | | Hz, 3H). | | | | | | |
| 263 | 2HCl | ¹H-NMR (400 MHz, DMSO-d₆): δ | DMSO | 95 | R1 | 415 | t = 1.430 min | Method A |
| | | 10.39 (s, 1H), 9.74 (s, 1H), 9.21 (d, | | | | (M + 1) | | (0.01% TFA) |
| | | J = 7.9 Hz, 1H), 9.00 (d, J = 4.7 | | | | | | |
| | | Hz, 1H), 8.83 (s, 1H), 8.35 (d, J = | | | | | | |
| | | 8.6 Hz, 1H), 8.23 (d, J = 8.7 Hz, | | | | | | |
| | | 1H), 7.96 (dd, J = 7.6, 5.3 Hz, 1H), | | | | | | |
| | | 7.83-7.75 (m, 1H), 7.64-7.54 | | | | | | |
| | | (m, 2H), 3.30 (d, J = 4.3 Hz, 3H). | | | | | | |
| 264 | 2HCl | ¹H-NMR (400 MHz, DMSO-d₆): δ | DMSO | 95 | R1 | 365.1 | t = 1.866 min | Method B |
| | | 9.66 (s, 1H), 9.36 (d, J = 8.1 Hz, | | | | (M + 1) | | (NH4—HCO3) |
| | | 1H), 9.15 (d, J = 4.1 Hz, 1H), 9.02 | | | | | | |
| | | (d, J = 5.2 Hz, 1H), 8.70 (s, 1H), | | | | | | |
| | | 8.41 (s, 1H), 8.18-8.10 (m, 1H), | | | | | | |
| | | 7.84 (m, 2H), 7.58 (dd, J = | | | | | | |
| | | 14.4, 8.0 Hz, (7.28 (td, J = 8.4, | | | | | | |
| | | 1.6 Hz, 1H), 3.21 (d, J = 4.2 Hz, 3H). | | | | | | |
| 265 | | ¹H-NMR (400 MHz, DMSO-d₆): δ | DMSO | 95 | R1 | 417.1 | t = 2.014 min | Method B |
| | | 9.66 (d, J = 1.47 Hz, 1H), 8.91 (d, | | | | (M + 1) | | (NH4—HCO3) |
| | | J = 2.2 Hz, 1H), 8.79-8.72 (m, | | | | | | |
| | | 3H), 8.33 (s, 1H), 7.66-7.33 (m, | | | | | | |
| | | 4H), 3.19 (d, J = 2.2 Hz, 3H). | | | | | | |
| 266 | | ¹H-NMR (400 MHz, DMSO-d₆): δ | DMSO | 95 | R1 | 417.0 | t = 2.011 min | Method B |
| | | 9.65 (s, 1H), 8.97-8.59 (m, 4H), | | | | (M + 1) | | (NH4—HCO3) |
| | | 8.26 (s, 1H), 7.79 (dd, J = 15.5, | | | | | | |
| | | 8.6 Hz, 1H), 7.66-7.22 (m, 3H), | | | | | | |
| | | 3.18 (d, J = 2.0 Hz, 3H). | | | | | | |
| 267 | | ¹H-NMR (400 MHz, DMSO-d₆): δ | DMSO | 95 | R1 | 443.0 | t = 1.869 min | Method B |
| | | 9.66 (s, 1H), 8.57 (s, 1H), 8.78 (d, | | | | (M + 1) | | (NH4—HCO3) |
| | | J = 7.6 Hz, 1H), 8.73 (dd, J = 7.2, | | | | | | |
| | | 3.2 Hz, 1H), 8.49 (s, 1H), 8.08 (s, | | | | | | |
| | | 1H), 7.78 (d, J = 6.8 Hz, 1H), 7.59 | | | | | | |
| | | 7.47 (m, 1H), 7.34-7.31), (m, 1H), | | | | | | |
| | | 3.19 (s, 3H). | | | | | | |
| 268 | 2HCl | ¹H-NMR (400 MHz, DMSO) δ | DMSO | 95 | R1 | 361.0 | t = 1.794 min | Method B |
| | | 10.32 (s, 1H), 9.73 (s, 1H), 9.17 (d, | | | | (M + 1) | | (NH4—HCO3) |
| | | J = 7.8 Hz, 1H), 9.00 (d, J = 4.6 Hz, | | | | | | |
| | | 1H), 8.63 (s, 1H), 8.36 (d, J = 8.5 Hz, | | | | | | |
| | | 1H), 8.10 (d, J = 8.6 Hz, 1H), 7.98- | | | | | | |
| | | 7.89 (m, 1H), 7.49 (d, J = 6.7 Hz, 1H), | | | | | | |
| | | 7.45-7.35 (m, 2H), 3.28 (d, J = 4.2 | | | | | | |
| | | Hz, 3H), 2.45 (s, 3H). | | | | | | |
| 269 | | ¹H-NMR (400 MHz, DMSO-d₆): δ | DMSO | 99 | R1 | 415.0 | t = 2.009 min | Method B |
| | | 9.65 (d, J = 1.2 Hz, 1H), 8.86 (d, J = | | | | (M + 1) | | (NH4—HCO3) |
| | | 4.4 Hz, 1H), 8.77 (d, J = 7.6 Hz, | | | | | | |
| | | 1H), 8.72 (dd, J = 4.8, 1.2 Hz, 1H), | | | | | | |
| | | 8.68 (d, J = 1.2 Hz, 1H), 8.24 | | | | | | |
| | | (s,1H), 7.79-7.75 (m, 2H), 7.62- | | | | | | |
| | | 7.56 (m, 2H), 7.32-7.27 (m, 1H), | | | | | | |
| | | 3.20 (d, J = 4.4 Hz, 3H). | | | | | | |
| 270 | | ¹H-NMR (400 MHz, DMSO-d₆): δ | DMSO | 98 | R1 | 433.1 | t = 1.884 min | Method B |
| | | 9.65 (s, 1H), 8.87 (d, J = 4.4 Hz, | | | | (M + 1) | | (NH4—HCO3) |
| | | 1H), 8.78 (d, J = 7.6 Hz, 1H), 8.72 | | | | | | |
| | | (d, J = 4.0 Hz, 1H), 8.54 (s, 1H), | | | | | | |
| | | 8.06 (s, 1H), 7.60-7.52 (m, 3H), | | | | | | |
| | | 7.41 (dd, J = 12.8, 7.6 Hz, 1H), | | | | | | |
| | | 3.18 (d, J = 4.4 Hz, 3H). | | | | | | |
| 271 | 2HCl | 1H NMR (300 MHz, DMSO) δ 10.29 | DMSO | 99 | R2 | 317 | 1.51 | Method D |
| | | (s, 1H), 9.63 (d, J = 2.1 Hz, 1H), | | | | (M + 1) | | |
| | | 9.03 (d, J = 7.7 Hz, 1H), 8.94 (d, J = | | | | | | |
| | | 3.6 Hz, 1H), 8.78 (s, 1H), 8.35- | | | | | | |
| | | 8.09 (m, 2H), 7.87 (dd, J = 8.1, | | | | | | |
| | | 6.0 Hz, 1H), 7.11 (d, J = 3.2 Hz, | | | | | | |
| | | 1H), 6.31 (d, J = 3.2 Hz, 1H), 3.28 | | | | | | |
| | | (d, J = 4.2 Hz, 3H), 2.40 (s, 3H). | | | | | | |
| 272 | | 1H NMR (300 MHz, DMSO) δ 9.48 | DMSO | 99 | R2 | 338 | 1.65 | Method D |
| | | (d, J = 2.0 Hz, 1H), 8.76-8.49 | | | | (M + 1) | | |
| | | (m, 3H), 8.28 (d, J = 8.1 Hz, 1H), | | | | | | |
| | | 8.22 (s, 1H), 8.12 (d, J = 7.9 Hz, | | | | | | |
| | | 1H), 8.02-7.85 (m, 2H), 7.73 (t, J = | | | | | | |
| | | 7.8 Hz, 1H), 7.61 (t, J = 7.8 Hz, | | | | | | |
| | | 1H), 7.50 (dd, J = 7.9, 4.8 Hz, | | | | | | |
| | | 1H), 3.17 (d, J = 4.2 Hz, 3H). | | | | | | |
| 273 | 2HCl | 1H NMR (300 MHz, DMSO) δ 10.30 | DMSO | 99 | R2 | 317 | 1.43 | Method D |
| | | (s, 1H), 9.62 (d, J = 1.8 Hz, 1H), | | | | (M + 1) | | |
| | | 9.12-8.81 (m, 2H), 8.52 (d, J = | | | | | | |
| | | 8.8 Hz, 1H), 8.27 (s, 1H), 8.02 (d, | | | | | | |
| | | J = 8.7 Hz, 1H), 7.85 (dd, J = 8.1, | | | | | | |
| | | 5.0 Hz, 1H), 7.23 (d, J = 3.3 Hz, | | | | | | |
| | | 1H), 6.36 (d, J = 2.9 Hz, 1H), 3.26 | | | | | | |
| | | (d, J = 4.3 Hz, 3H), 2.40 (s, 3H). | | | | | | |
| 274 | | 1H NMR (300 MHz, DMSO) δ 9.51 | DMSO | 99 | R2 | 331 | 1.77 | Method D |
| | | (s, 1H), 8.77-8.60 (m, 2H), 8.55 | | | | (M + 1) | | |
| | | (d, J = 4.9 Hz, 1H), 8.26 (d, J = | | | | | | |
| | | 7.9 Hz, 1H), 7.87 (d, J = 7.4 Hz, | | | | | | |
| | | 1H), 7.74-7.45 (m, 5H), 7.26 (t, J = | | | | | | |
| | | 7.8 Hz, 1H), 3.17 (d, J = 4.2 Hz, 3H). | | | | | | |
| 275 | 2HCl | 1H NMR (300 MHz, DMSO) δ 10.38 | DMSO | 97 | R2 | 317 | 1.36 | Method D |
| | | (s, 1H), 9.68 (s, 1H), 9.05 (d, J = | | | | (M + 1) | | |
| | | 6.8 Hz, 1H), 8.95 (d, J = 3.5 Hz, | | | | | | |
| | | 1H), 8.57 (d, J = 8.6 Hz, 1H), 8.39 | | | | | | |
| | | (s, 1H), 8.06-7.80 (m, 2H), 7.70 | | | | | | |
| | | (s, 1H), 6.93 (s, 1H), 3.29 (d, J = | | | | | | |
| | | 3.4 Hz, 3H), 2.60 (s, 3H). | | | | | | |
| 276 | 2HCl | 1H NMR (300 MHz, DMSO) δ 10.37 | DMSO | 98 | R2 | 331 | 1.44 | Method D |
| | | (s, 1H), 9.67 (d, J = 1.7 Hz, 1H), | | | | (M + 1) | | |
| | | 9.05 (d, J = 8.0 Hz, 1H), 8.94 (dd, | | | | | | |
| | | J = 5.0, 1.5 Hz, 1H), 8.54 (d, J = | | | | | | |
| | | 8.6 Hz, 1H), 8.35 (s, 1H), 8.03- | | | | | | |
| | | 7.71 (m, 2H), 6.50 (s, 1H), 3.28 | | | | | | |
| | | (d, J = 4.5 Hz, 3H), 2.54 (s, 3H), | | | | | | |
| | | 2.28 (s, 3H). | | | | | | |
| 277 | | 1H NMR (300 MHz, DMSO) δ 9.51 | DMSO | 97 | R2 | 349 | 2.31 | Method C |
| | | (s, 1H), 8.81-8.49 (m, 3H), 8.28 | | | | (M + 1) | | |
| | | (d, J = 8.5 Hz, 1H), 7.91 (d, J = | | | | | | |
| | | 7.0 Hz, 1H), 7.73-7.43 (m, 4H), | | | | | | |
| | | 7.30 (t, J = 9.5 Hz, 1H), 3.17 (d, J = | | | | | | |
| | | 4.2 Hz, 3H). | | | | | | |
| 278 | | 1H NMR (300 MHz, DMSO) δ 9.51 | DMSO | 97 | R2 | 349 | 1.85 | Method D |
| | | (s, 1H), 8.77-8.60 (m, 2H), 8.56 (d, J = | | | | (M + 1) | | |
| | | 4.2 Hz, 1H), 8.25 (d, J = 8.2 Hz, 1H), | | | | | | |
| | | 8.00-7.82 (m, 2H), 7.75-7.48 | | | | | | |
| | | (m, 4H), 3.17 (d, J = 4.4 Hz, 3H). | | | | | | |
| 279 | 2HCl | 1H NMR (300 MHz, DMSO) δ 9.95 | DMSO | 96 | R2 | 361 | 1.57 | Method D |
| | | (s, 1H), 9.63 (s, 1H), 9.02 (d, J = | | | | (M + 1) | | |
| | | 7.2 Hz, 1H), 8.91 (d, J = 5.1 Hz, | | | | | | |
| | | 1H), 8.51 (d, J = 8.3 Hz, 1H), 8.40 | | | | | | |
| | | (s, 1H), 8.06 (d, J = 8.5 Hz, 1H), | | | | | | |
| | | 7.95-7.75 (m, 1H), 7.31-7.11 | | | | | | |
| | | (m, 2H), 6.97 (d, J = 9.2 Hz, 1H), | | | | | | |
| | | 3.87 (s, 3H), 3.26 (d, J = 3.6 Hz, 3H). | | | | | | |
| 280 | 2HCl | 1H NMR (300 MHz, DMSO) δ 10.51 | DMSO | 98 | R2 | 317 | 1.44 | Method D |
| | | (s, 1H), 9.66 (s, 1H), 9.06 (d, J = | | | | (M + 1) | | |
| | | 7.0 Hz, 1H), 8.95 (dd, J = 5.0, 1.4 | | | | | | |
| | | Hz, 1H) 8.65 (s, 1H) 8.25 (d, J = | | | | | | |
| | | 8.7 Hz, 1H), 8.12 (d, J = 8.4 Hz, | | | | | | |
| | | 1H), 7.88 (dd, J = 8.1, 4.9 Hz, | | | | | | |
| | | 1H), 7.79-7.60 (m, 1H), 6.96 (d, | | | | | | |
| | | J = 1.9 Hz, 1H), 3.29 (d, J = 3.5 | | | | | | |
| | | Hz, 3H), 2.55 (s, 3H). | | | | | | |
| 281 | 2HCl | 1H NMR (300 MHz, DMSO) δ 9.63 | DMSO | 94 | R2 | 349 | 1.49 | Method D |
| | | (s, 1H), 9.01 (d, J = 7.3 Hz, 1H), 8.98- | | | | (M + 1) | | |
| | | 8.84 (m, 1H), 8.18 (d, J = 7.2 Hz 1H) | | | | | | |
| | | 8.10-7.94 (m, 1H), 7.93-7.80 (m, | | | | | | |
| | | 1H), 7.79-7.67 (m, 1H), 7.67- | | | | | | |
| | | 7.45 (m, 2H), 7.45 7.26 (m, 1H), | | | | | | |
| | | 7.09 (s, 1H), 3.04 (d, J = 2.7 Hz, 3H). | | | | | | |
| 282 | 3HCl | 1H NMR (300 MHz, DMSO) δ 9.92 | DMSO | 99 | R2 | 332 | 1.89 | Method C |
| | | (s, 1H), 9.64 (d, J = 1.4 Hz, 1H), | | | | (M + 1) | | |
| | | 9.10 (d, J = 7.6 Hz, 1H), 9.01-8.89 | | | | | | |
| | | (m, 2H), 8.70 (d, J = 2.6 Hz, 1H), | | | | | | |
| | | 8.57 (d, J = 8.7 Hz, 1H), 8.48 (s, | | | | | | |
| | | 1H), 8.25 (d, J = 10.2 Hz, 1H), | | | | | | |
| | | 8.11 (d, J = 8.6 Hz, 1H), 7.97- | | | | | | |
| | | 7.83 (m, 1H), 3.25 (d, J = 4.2 Hz, 3H). | | | | | | |
| 283 | 3HCl | 1H NMR (300 MHz, DMSO) δ 9.62 | DMSO | 99 | R2 | 350 | 2.14 | Method C |
| | | (d, J = 1.3 Hz, 1H), 8.82-8.72 | | | | (M + 1) | | |
| | | (m, 2H), 8.72-8.57 (m, 2H), 8.31 | | | | | | |
| | | (d, J = 8.8 Hz, 1H), 7.84 (d, J = | | | | | | |
| | | 8.8 Hz, 1H), 7.68 (s, 2H), 7.53 | | | | | | |
| | | (dd, J = 7.9, 4.8 Hz, 1H), 3.18 (d, | | | | | | |
| | | J = 4.5 Hz, 3H). | | | | | | |
| 284 | 3HCl | 1H NMR (300 MHz, DMSO) δ 9.63 | DMSO | 99 | R2 | 350 | 2.08 | Method C |
| | | (d, J = 1.4 Hz, 1H), 8.76 (d, J = | | | | (M + 1) | | |
| | | 8.0 Hz, 1H), 8.69 (d, J = 4.6 Hz, | | | | | | |
| | | 1H), 8.63 (d, J = 4.6 Hz, 1H), 8.36 | | | | | | |
| | | (d, J = 8.6 Hz, 1H), 8.29 (d, J = | | | | | | |
| | | 1.3 Hz, 1H), 8.01 (dd, J = 8.6, 1.5 | | | | | | |
| | | Hz, 1H), 7.79 (s, 2H), 7.55 (dd, J = | | | | | | |
| | | 7.9, 4.8 Hz, 1H), 3.16 (d, J = | | | | | | |
| | | 3.8 Hz, 3H). | | | | | | |
| 285 | 2HCl | 1H NMR (300 MHz, DMSO) δ 10.30 | DMSO | 99 | R2 | 332 | 1.41 | Method D |
| | | (s, 1H), 9.62 (d, J = 1.6 Hz, 1H), | | | | (M + 1) | | |
| | | 8.99 (d, J = 7.4 Hz, 1H), 8.93 (dd, | | | | | | |
| | | J = 5.0, 1.5 Hz, 1H), 8.52 (s, 1H), | | | | | | |
| | | 8.18 (d, J = 8.6 Hz, 1H), 8.06 | | | | | | |
| | | (d, J = 8.5 Hz, 1H), 7.85 (dd, J = 7.9, | | | | | | |
| | | 4.9 Hz, 1H), 6.51 (s, 1H), 3.29 (d, | | | | | | |
| | | J = 4.4 Hz, 3H), 2.48 (s, 3H), 2.28 | | | | | | |
| | | (s, 3H). | | | | | | |
| 286 | 2HCl | ¹H NMR (400 MHz, DMSO) δ 9.74 | DMSO | >98 | R6 | | | |
| | | (d, J = 1.8 Hz, 1H), 9.37 (d, J = | | | | | | |
| | | 8.0 Hz, 1H), 8.97 (d, J = 5.4 Hz, | | | | | | |
| | | 1H), 8.80 (brs, 1H), 8.23 (d, J = | | | | | | |
| | | 8.6 Hz, 1H), 8.14-8.00 (m, 1H), | | | | | | |
| | | 7.57-7.40 (m, 2H), 7.34 (dd, J = | | | | | | |
| | | 15.3, 7.1 Hz, 1H), 3.21 (d, J = 4.4 | | | | | | |
| | | Hz, 3H), 2.54 (s, 3H). 1H of 2HCl | | | | | | |
| | | was not observed | | | | | | |
| 287 | 2HCl | ¹H NMR (400 MHz, DMSO) δ 9.73 | DMSO | >98 | R6 | | | |
| | | (d, J = 1.8 Hz, 1H), 9.44 (d, J = | | | | | | |
| | | 8.3 Hz, 1H), 9.01 (d, J = 4.4 Hz, | | | | | | |
| | | 1H), 8.85 (s, 1H), 8.24 (d, J = 8.5 | | | | | | |
| | | Hz, 1H), 8.15 (dd, J = 8.0, 5.7 Hz, | | | | | | |
| | | 1H), 7.84-7.57 (m, 2H), 7.46 (d, | | | | | | |
| | | J = 8.5 Hz, 1H), 3.21 (d, J = 4.3 | | | | | | |
| | | Hz, 3H), 2.56 (s, 3H). 1H of 2HCl | | | | | | |
| | | was not observed | | | | | | |
| 288 | HCl | ¹H NMR (400 MHz, DMSO) δ 9.73 | DMSO | >98 | R6 | | | |
| | | (d, J = 1.8 Hz, 1H), 9.33 (dd, J = | | | | | | |
| | | 5.7, 4.1 Hz, 1H), 8.95 (dd, J = 5.4, | | | | | | |
| | | 1.4 Hz, 1H), 8.79 (d, J = 4.4 Hz, | | | | | | |
| | | 1H), 8.24 (d, J = 8.5 Hz, 1H), 8.05 | | | | | | |
| | | (dd, J = 8.0, 5.4 Hz, 1H), 7.79- | | | | | | |
| | | 7.58 (m, 1H), 7.48 (d, J = 8.5 Hz, | | | | | | |
| | | 1H), 7.27 (ddd, J = 8.0, 5.0, 2.6 | | | | | | |
| | | Hz, 1H), 3.20 (d, J = 4.4 Hz, 3H), | | | | | | |
| | | 2.57 (s, 3H). 1H of HCl was not | | | | | | |
| | | observed. | | | | | | |
| 289 | HCl | ¹H NMR (400 MHz, DMSO) δ 9.73 | DMSO | >98 | R6 | | | |
| | | (d, J = 1.7 Hz, 1H), 9.29 (d, J = | | | | | | |
| | | 7.8 Hz, 1H), 8.93 (d, J = 5.3 Hz, | | | | | | |
| | | 1H), 8.73 (s, 1H), 8.20 (d, J = 8.6 | | | | | | |
| | | Hz, 1H), 8.07-7.94 (m, 1H), 7.58- | | | | | | |
| | | 7.41 (m, 3H), 3.20 (d, J = 4.4 | | | | | | |
| | | Hz, 3H), 2.65 (s, 3H). 1H of HCl | | | | | | |
| | | was not observed. | | | | | | |
| 290 | HCl | ¹H NMR (400 MHz, DMSO) δ 9.70 | DMSO | >98 | R6 | | | |
| | | (s, 1H), 9.20 (d, J = 8.2 Hz, 1H), | | | | | | |
| | | 8.90 (d, J = 5.2 Hz, 1H), 8.17 (d, J = | | | | | | |
| | | 8.7 Hz, 1H), 8.00-7.87 (m, 3H), | | | | | | |
| | | 7.81 (t, J = 12.1 Hz, 1H), 7.75 (t, J = | | | | | | |
| | | 7.7 Hz, 1H), 7.42 (d, J = 8.7 Hz, 1H), | | | | | | |
| | | 3.50 (s, 6H), 2.64 (s, 3H). 1H of | | | | | | |
| | | HCl was not observed. | | | | | | |
| 291 | 2HCl | ¹H NMR (400 MHz, DMSO) δ 9.70 | DMSO | >98 | R6 | | | |
| | | (s, 1H), 9.23 (s, 1H), 8.91 (s, (H), | | | | | | |
| | | 8.18 (d, J = 8.7 Hz, 1H), 8.01 (d, J = | | | | | | |
| | | 8.2 Hz, 3H), 7.69 (d, J = 8.1 Hz, | | | | | | |
| | | 2H), 7.41 (d, J = 8.7 Hz, 1H), 3.49 | | | | | | |
| | | (s, 6H), 2.64 (s, 3H). 1H of 2HCl | | | | | | |
| | | was not observed. | | | | | | |
| 292 | HCl | ¹H NMR (400 MHz, DMSO) δ 9.70 | DMSO | >98 | R6 | | | |
| | | (s, 1H), 9.36 (d, J = 7.9 Hz, 1H), | | | | | | |
| | | 8.98 (d, J = 5.4 Hz, 1H), 8.16 (d, J = | | | | | | |
| | | 8.7 Hz, 1H), 8.13-8.03 (m, 1H), | | | | | | |
| | | 7.49 (dd, J = 20.1, 13.2 Hz, 2H), | | | | | | |
| | | 7.42 (d, J = 8.7 Hz, 1H), 3.50 | | | | | | |
| | | (s, 6H), 2.67 (s, 3H). 1H of HCl | | | | | | |
| | | was not observed | | | | | | |
| 293 | 2HCl | ¹H NMR (400 MHz, DMSO) δ 9.73 | DMSO | >98 | R6 | | | |
| | | (s, 1H), 9.23 (d, J = 8.5 Hz, 1H), | | | | | | |
| | | 8.95 (d, J = 6.3 Hz, 2H), 8.90 (d, J = | | | | | | |
| | | 5.3 Hz, 1H), 8.78 (s, 1H), 8.29 | | | | | | |
| | | (d, J = 8.5 Hz, 1H), 7.96 (m, 3H), | | | | | | |
| | | 7.52 (d, J = 8.5 Hz, 1H), 3.21 (d, J = | | | | | | |
| | | 4.3 Hz, 3H), 2.68 (s, 3H). 1H of | | | | | | |
| | | 2HCl was not observed. | | | | | | |
| 294 | 2HCl | ¹H NMR (400 MHz, DMSO) δ 9.62 | DMSO | >98 | R6 | | | |
| | | (s, 1H), 9.24 (s, 1H), 8.96 (s, 1H), | | | | | | |
| | | 8.24 (brm, 3H), 8.06 (s, 1H), 7.59- | | | | | | |
| | | 7.35 (m, 3H), 2.66 (s, 3H). 1H of | | | | | | |
| | | 2HCl was not observed. | | | | | | |
| 295 | 3HCl | ¹H NMR (400 MHz, DMSO) δ 9.74 | DMSO | >98 | R6 | | | |
| | | (s, 1H), 9.34 (d, J = 8.3 Hz, 1H), | | | | | | |
| | | 8.96 (d, J = 5.4 Hz, 1H), 8.91 (d, J = | | | | | | |
| | | 6.0 Hz, 2H), 8.34 (d, J = 8.6 Hz, | | | | | | |
| | | 1H), 8.11-7.97 (m, 3H), 7.54 (d, | | | | | | |
| | | J = 8.5 Hz, 1H), 3.21 (d, J = 4.3 | | | | | | |
| | | Hz, 3H), 2.82 (s, 3H), 2.70 (s, 3H). | | | | | | |
| | | 1H of 3 HCl was not observed | | | | | | |
| 296 | 2HCl | ¹H NMR (400 MHz, DMSO) δ 9.72 | DMSO | >98 | R6 | | | |
| | | (s, 1H), 9.21 (s, 1H), 8.90 (s, 1H), | | | | | | |
| | | 8.66 (s, 1H), 8.16 (d, J = 8.6 Hz, | | | | | | |
| | | 1H), 7.95 (s, 1H), 7.42 (d, J = 7.9 | | | | | | |
| | | Hz, 1H), 7.38-7.26 (m, 4H), 3.20 | | | | | | |
| | | (d, J = 4.2 Hz, 3H), 2.64 (s, 3H), | | | | | | |
| | | 2.40 (s, 3H). 1H of 2HCl | | | | | | |
| | | was not observed. | | | | | | |
| 297 | 2HCl | ¹H NMR (400 MHz, DMSO) δ 9.72 | DMSO | >98 | R6 | | | |
| | | (s, 1H), 9.25 (s, 1H), 8.91 (d, J = | | | | | | |
| | | 4.1 Hz, 1H), 8.70 (s, 1H), 8.17 (d, | | | | | | |
| | | J = 8.4 Hz, 1H), 8.05-7.86 (m, | | | | | | |
| | | 1H), 7.48-7.25 (m, 4H), 7.16 (d, | | | | | | |
| | | J = 6.8 Hz, 1H), 3.21 (d, J = 4.4 | | | | | | |
| | | Hz, 3H), 2.42 (s, 3H), 2.03 (s, 3H). | | | | | | |
| | | 1H of 2HCl was not observed. | | | | | | |
| 298 | 2HCl | ¹H NMR (400 MHz, DMSO) δ 9.73 | DMSO | >98 | R6 | | | |
| | | (s, 1H), 9.33 (d, J = 8.0 Hz, 1H), | | | | | | |
| | | 8.95 (d, J = 5.0 Hz, 1H), 8.75 | | | | | | |
| | | (brs, 1H), 8.20 (d, J = 8.5 Hz, 1H), | | | | | | |
| | | 8.13 (s, 1H), 8.08-7.98 (m, 1H), | | | | | | |
| | | 7.82 (s, 1H), 7.77 (d, J = 8.7 Hz, | | | | | | |
| | | 1H), 7.59-7.34 (m, 2H), 4.12 (s, | | | | | | |
| | | 3H), 3.22 (d, J = 4.3 Hz, | | | | | | |
| | | 3H), 2.66 (s, 3H). 1H of 2HCl was not | | | | | | |
| | | observed. | | | | | | |
| 299 | 2HCl | ¹H NMR (400 MHz, DMSO) δ 10.04 | DMSO | >98 | R4 | | | |
| | | (s, 1H), 9.65 (s, 1H), 9.11-8.91 | | | Temperature | | | |
| | | (m, 2H), 8.41 (s, 1H), 8.20-7.99 | | | at 100° C. | | | |
| | | (m, 1H), 7.95-7.79 (m, 1H), 7.59 | | | | | | |
| | | (dd, J = 14.1, 8.0 Hz, 1H), 7.45- | | | | | | |
| | | 7.25 (m, 3H), 3.27 (d, J = 4.6 Hz, | | | | | | |
| | | 3H), 2.47 (s, 3H). | | | | | | |
| 300 | 2HCl | ¹H NMR (400 MHz, DMSO) δ 9.58 | DMSO | >98 | R4 | | | |
| | | (d, J = 6.7 Hz, 1H), 8.98-8.88 (m, 2H), | | | Temperature | | | |
| | | 8.34-8.29 (m, 1H), 7.93-7.79 (m, 2H), | | | at 100° C. | | | |
| | | 7.60-7.49 (m, 2H), 3.25 (d, J = 4.5 Hz, | | | | | | |
| | | 3H), 2.48 (s, 3H). | | | | | | |
| 301 | 2HCl | ¹H NMR (400 MHz, DMSO) δ 9.91 | DMSO | >98 | R4 | | | |
| | | (s, 1H), 9.63 (d, J = 2.0 Hz, 1H), 9.09- | | | Temperature | | | |
| | | 8.91 (m, 2H), 8.42 (s, 1H), 8.05 (s, | | | at 100° C. | | | |
| | | 1H), 7.95-7.84 (m, 1H), 7.60-7.50 | | | | | | |
| | | (m, 1H), 7.43-7.32 (m, 1H), 3.25 | | | | | | |
| | | (d, J = 4.5 Hz, 3H), 2.37 (s, 3H). | | | | | | |
| 302 | HCl | ¹H NMR (400 MHz, DMSO) δ 10.13 | DMSO | >98 | R4 | | | |
| | | (s, 1H), 9.68 (d, J = 1.6 Hz, 1H), 9.08 | | | Temperature | | | |
| | | (d, J = 8.2 Hz, 1H), 9.01-8.94 (m, | | | at 100° C. | | | |
| | | 1H), 8.31 (s, 1H), 8.20 (s, 1H), 7.92- | | | | | | |
| | | 7.84 (m, 1H), 7.32-7.12 (m, 3H), | | | | | | |
| | | 3.25 (d, J = 4.5 Hz, 3H), 2.22 (s, | | | | | | |
| | | 3H), 2.07 (s, 3H). | | | | | | |
| 303 | HCl | ¹H NMR (400 MHz, DMSO) δ 10.02 | DMSO | >98 | R4 | | | |
| | | (s, 1H), 9.66 (d, J = 1.6 Hz, 1H), | | | Temperature | | | |
| | | 9.14-8.91 (m, 2H), 8.43 (s, 1H), | | | at 100° C. | | | |
| | | 8.19-8.04 (m, 1H), 7.99-7.81 | | | | | | |
| | | (m, 3H), 7.79-7.63 (m, 1H), 3.27 | | | | | | |
| | | (d, J = 4.5 Hz, 3H), 2.45 (s, 3H). | | | | | | |
| 304 | HCl | ¹H NMR (400 MHz, DMSO) δ 10.07 | DMSO | >98 | R4 | | | |
| | | (s, 1H), 9.63 (d, J = 1.7 Hz, 1H), | | | Temperature | | | |
| | | 9.09-8.90 (m, 2H), 8.42 (s, 1H), 8.15- | | | at 100° C. | | | |
| | | 8.01 (m, 1H), 7.93-7.79 (m, 1H), 7.37 | | | | | | |
| | | (dd, J = 11.5, 8.3 Hz, 1H), 7.27 (dd, | | | | | | |
| | | J = 8.3, 2.0 Hz, 1H), 7.10-6.98 (m, | | | | | | |
| | | 1H), 3.92 (s, 3H), 3.27 (d, J = 4.5 | | | | | | |
| | | Hz, 3H), 2.48 (s, 3H). | | | | | | |
| 305 | 2HCl | ¹H NMR (400 MHz, DMSO) δ 9.58 | DMSO | >98 | R3 | | | |
| | | (s, 1H), 9.13 (d, J = 6.6 Hz, 2H), | | | Temperature | | | |
| | | 8.93 (d, J = 4.0 Hz, 1H), 8.27 (s, 1H), | | | at 80° C. | | | |
| | | 8.04-7.94 (m, 2H), 7.94-7.86 (m, | | | | | | |
| | | 1H), 7.68 (s, 1H), 7.58 (t, J = 7.9 | | | | | | |
| | | Hz, 1H), 7.54-7.46 (m, 1H), 4.12 (s, | | | | | | |
| | | 3H), 3.22 (d, J = 4.4 Hz, 3H). | | | | | | |
| 306 | HCl | ¹H NMR (400 MHz, DMSO) δ 9.60 | DMSO | >98 | R3 | | | |
| | | (d, J = 1.6 Hz, 1H), 9.12 (d, J = 7.9 | | | Temperature | | | |
| | | Hz, 1H), 8.98-8.86 (m, 2H), 8.00- | | | at 80° C. | | | |
| | | 7.89 (m, 2H), 7.69-7.61 (m, 1H), | | | | | | |
| | | 7.61-7.55 (m, 1H), 7.54- 7.46 (m, | | | | | | |
| | | 2H), 7.45 (d, J = 1.2 Hz, 1H), 4.03 | | | | | | |
| | | (s, 3H), 3.18 (d, J = 4.5 Hz, 3H). | | | | | | |
| 307 | 2HCl | ¹H NMR (400 MHz, DMSO) δ 9.56 | DMSO | >8 | R3 | | | |
| | | (d, J = 1.6 Hz, 1H), 9.36 (s, 1H), 9.19 | | | Temperature | | | |
| | | (d, J = 7.8 Hz, 1H), 8.98 (dd, J = 5.4, | | | at 80° C. | | | |
| | | 1.4 Hz, 1H), 8.31 (d, J = 1.3 Hz, | | | | | | |
| | | 1H), 8.06 (dd, J = 8.0, 5.6 Hz, 1H), | | | | | | |
| | | 8.01-7.92 (m, 2H), 7.69 (d, J = 1.2 | | | | | | |
| | | Hz, 1H), 7.65-7.57(m, 2H), 4.12 (s, | | | | | | |
| | | 3H), 3.22 (d, J = 4.3 Hz, 3H). | | | | | | |
| 308 | 2 HCl | 1H NMR (300 MHz, DMSO) δ 9.73- | DMSO | 96 | R2 | 367.5 | 2.57 | Method C |
| | | 9.39 (m, 1H), 9.05-8.79 (m, 2H), | | | | (M + 1) | | |
| | | 8.56-8.36 (m, 1H), 8.31-8.18 (m, 1H), | | | | | | |
| | | 8.09-7.96 (m, 1H), 7.96-7.57 (m, | | | | | | |
| | | 3H), 7.39-7.08 (m, 2H), 4.07-3.24 | | | | | | |
| | | (m, 6H), 2.11-1.89 (m, 1H), | | | | | | |
| | | 1.13-0.90 (m, 2H), 0.88-0.61 (m, 2H). | | | | | | |
| 309 | 2 HCl | 1H NMR (400 MHz, DMSO) δ 9.55 | DMSO | >98 | R3 | | | |
| | | (d, J = 1.7 Hz, 1H), 9.36 (br-s, 1H), | | | Temperature | | | |
| | | 9.14 (d, J = 7.7 Hz, 1H), 8.96 (dd, J = | | | at 100° C. | | | |
| | | 5.4, 1.4 Hz, 1H), 8.26 (d, J = 1.5 Hz, | | | | | | |
| | | 1H), 8.03 (dd, J = 8.1, 5.6 Hz, 1H), | | | | | | |
| | | 7.90-7.80 (m, 2H), 7.69 (d, J = 1.3 | | | | | | |
| | | Hz, 1H), 7.44-7.32 (m, 2H), 4.12 | | | | | | |
| | | (s, 3H), 3.23 (d, J = 4.4 Hz, 3H), | | | | | | |
| | | 2.40 (s, 3H). | | | | | | |
| 310 | 2 HCl | 1H NMR (400 MHz, DMSO) δ 9.56 | DMSO | >98 | R3 | | | |
| | | (d, J = 1.7 Hz, 1H), 9.35 (br-s, | | | Temperature | | | |
| | | 1H), 9.16 (d, J = 8.2 Hz, 1H), 8.97 | | | at 100° C. | | | |
| | | (dd, J = 5.4, 1.4 Hz, 1H), 8.25 (d, | | | | | | |
| | | J = 1.4 Hz, 1H), 8.04 (dd, J = 8.0, | | | | | | |
| | | 5.5 Hz, 1H), 7.79-7.63 (m, 3H), 7.44 | | | | | | |
| | | (dd, J = 7.6 Hz, 1H), 7.28 (d, J = 7.5 | | | | | | |
| | | Hz, 1H), 4.13 (s, 3H), 3.24 (d, J = 4.4 | | | | | | |
| | | Hz, 3H), 2.45 (s, 3H). | | | | | | |
| 311 | | 1H NMR (400 MHz, DMSO) δ 12.83 | DMSO | >98 | R3 | | | |
| | | (s, 1H), 9.31 (s, 1H), 8.78 (m, 1H), | | | Temperature | | | |
| | | 8.51 (d, J = 7.6 Hz, 1H), 7.81- | | | at 100° C. | | | |
| | | 7.44 (m, 3H), 7.44-7.22 (m, 4H), 3.99 | | | | | | |
| | | (s, 3H), 2.31 (s, 3H). | | | | | | |
| 312 | | 1H NMR (400 MHz, DMSO) δ 9.58 | DMSO | >98 | Method L | | | |
| | | (dd, J = 2.1, 0.8 Hz, 1H), 8.76-8.64 | | | Temperature | | | |
| | | (m, 2H), 8.37 (d, 1H), 8.12 (d, J = 1.9 | | | at 100° C. | | | |
| | | Hz, 1H), 8.04 (brs, 2H), 7.98-7.91 (m, | | | | | | |
| | | 2H), 7.89 (dd, J = 8.6, 1.9 Hz, 1H), | | | | | | |
| | | 7.55 (ddd, J = 7.9, 4.8, 0.8 Hz, 1H). | | | | | | |
| 313 | | 1H NMR (400 MHz, DMSO) δ 9.62 | DMSO | >98 | Method L | | | |
| | | (dd, J = 2.2, 0.8 Hz, 1H), 8.77-8.73 | | | Temperature | | | |
| | | (m, 1H), 8.69 (dd, J = 4.8, 1.7 Hz, | | | at 100° C. | | | |
| | | 1H), 8.29 (d, J = 8.8 Hz, 1H), 8.16 (d, | | | | | | |
| | | J = 2.0 Hz, 1H), 7.99-7.86 (m, 2H), | | | | | | |
| | | 7.82 (dd, J = 8.8, 2.1 Hz, 1H), 7.56 | | | | | | |
| | | (ddd, J = 8.0, 4.8, 0.8 Hz, | | | | | | |
| | | 1H), 3.47 (s, 6H). | | | | | | |
| 314 | 3 HCl | 1H NMR (400 MHz, DMSO) δ 11.13 | DMSO | >98 | Method L | | | |
| | | (brs, 1H), 9.74 (d, J = 1.7 Hz, 1H), | | | Temperature | | | |
| | | 9.28 (d, J = 8.1 Hz, 1H), 9.00 (dd, | | | at 100° C. | | | |
| | | J = 5.2, 1.5 Hz, 1H), 8.63-8.42 | | | | | | |
| | | (m, 2H), 7.98 (dd, J = 7.9, 5.3 Hz, | | | | | | |
| | | 1H), 7.88 (d, J = 8.9 Hz, 1H), 7.61 | | | | | | |
| | | (ddd, J = 9.1, 6.1, 3.1 Hz, 1H), | | | | | | |
| | | 7.58-7.47 (m, 1H), 7.47-7.38 | | | | | | |
| | | (m, 1H), 4.21-4.11 (m, 2H), 3.73 | | | | | | |
| | | (s, 3H), 3.28-3.19 (m, 2H), 2.75 | | | | | | |
| | | (d, J = 4.9 Hz, 6H), 2.36-2.25 (m, 2H). | | | | | | |
| 315 | | 1H NMR (400 MHz, DMSO) δ 9.65 | DMSO | >98 | Method L | | | |
| | | (dd, J = 2.1, 0.8 Hz, 1H), 8.81- | | | Temperature | | | |
| | | 8.75 (m, 1H), 8.69 (dd, J = 4.7, | | | at 100° C. | | | |
| | | 1.7 Hz, 1H), 8.59-8.51 (m, 1H), | | | | | | |
| | | 8.29 (d, J = 8.4 Hz, 1H), 7.71 (d, J = | | | | | | |
| | | 1.6 Hz, 1H), 7.59-7.50 (m, 2H), 7.43- | | | | | | |
| | | 7.35 (m, 1H), 7.24-7.15 (m, 2H), | | | | | | |
| | | 3.19 (d, J = 4.5 Hz, 3H), 2.26 (s, 3H). | | | | | | |
| 316 | | 1H NMR (400 MHz, DMSO) δ 9.65 | DMSO | >98 | Method L | | | |
| | | (d, J = 1.5 Hz, 1H), 8.84-8.73 (m, | | | Temperature | | | |
| | | 1H), 8.69 (dd, J = 4.7, 1.7 Hz, 1H), | | | at 100° C. | | | |
| | | 8.50 (d, J = 4.5 Hz, 1H), 8.25 (d, | | | | | | |
| | | J = 8.6 Hz, 1H), 7.89 (d, J = 1.6 Hz, | | | | | | |
| | | 1H), 7.65 (dd, J = 8.5, 1.7 Hz, 1H), | | | | | | |
| | | 7.55 (dd, J = 7.4, 4.7 Hz, 1H), 7.36 | | | | | | |
| | | (dd, J = 9.3, 3.1 Hz, 1H), 7.32-7.23 | | | | | | |
| | | (m, 1H), 7.19 (dd, J = 9.1, 4.7 Hz, | | | | | | |
| | | 1H), 3.81 (s, 3H), 3.18 (d, J = 4.5 | | | | | | |
| | | Hz, 3H). | | | | | | |
| 317 | | 1H NMR (400 MHz, DMSO) δ 9.64 | DMSO | >98 | Method L | | | |
| | | (dd, J = 2.1, 0.8 Hz, 1H), 8.81-8.74 | | | Temperature | | | |
| | | (m, 1H), 8.69 (dd, J = 4.7, 1.7 Hz, 1H), | | | at 100° C. | | | |
| | | 8.53-8.43 (m, 1H), 8.26 (d, J = 8.6 | | | | | | |
| | | Hz, 1H), 8.04 (d, J = 1.7 Hz, 1H), | | | | | | |
| | | 7.92-7.84 (m, 2H), 7.55 (ddd, J = | | | | | | |
| | | 8.0, 4.8, 0.8 Hz, 1H), 7.29 (dd, J = | | | | | | |
| | | 3.4, 0.5 Hz, 1H), 6.70 (dd, J = 3.4, | | | | | | |
| | | 1.8 Hz, 1H), 3.16 (d, J = 4.5 Hz, 3H). | | | | | | |
| 318 | | 1H NMR (400 MHz, DMSO) δ 9.64 | DMSO | >98 | Method L | | | |
| | | (dd, J = 2.1, 0.8 Hz, 1H), 8.84-8.73 | | | Temperature | | | |
| | | (m, 1H), 8.69 (dd, J = 4.7, 1.7 Hz, 1H), | | | at 100° C. | | | |
| | | 8.54-8.34 (m, 2H), 8.23 (d, J = 8.6 Hz, | | | | | | |
| | | 1H), 8.02 (d, J = 1.7 Hz, 1H), 7.92- | | | | | | |
| | | 7.73 (m, 2H), 7.55 (ddd, J = 8.0, | | | | | | |
| | | 4.8, 0.8 Hz, 1H), 7.21 (dd, J = | | | | | | |
| | | 1.9, 0.8 Hz, 1H), 3.16 (d, J = 4.5 Hz, 3H). | | | | | | |
| 319 | | 1H NMR (400 MHz, DMSO) δ 9.65 | DMSO | >98 | Method L | | | |
| | | (dd, J = 2.1, 0.8 Hz, 1H), 8.85-8.74 | | | Temperature | | | |
| | | (m, 1H), 8.69 (dd, J = 4.7, 1.7 Hz, | | | at 100° C. | | | |
| | | 1H), 8.57-8.38 (m, 1H), 8.26 (d, J = | | | | | | |
| | | 8.6 Hz, 1H), 7.99 (d, J = 1.8 Hz, | | | | | | |
| | | 1H), 7.86 (dd, J = 8.6, 1.9 Hz, 1H), | | | | | | |
| | | 7.82 (dd, J = 3.6, 1.1 Hz, 1H), 7.70 | | | | | | |
| | | (dd, J = 5.1, 1.1 Hz, 1H), 7.55 (ddd, | | | | | | |
| | | J = 8.0, 4.8, 0.8 Hz, 1H), 7.23 (dd, | | | | | | |
| | | J = 5.1, 3.6 Hz, 1H), 3.16 (d, J = 4.5 | | | | | | |
| | | Hz, 3H). | | | | | | |
| 320 | | 1H NMR (400 MHz, DMSO) δ 9.65 | DMSO | >98 | Method L | | | |
| | | (dd, J = 2.1, 0.8 Hz, 1H), 8.84-8.74 | | | Temperature | | | |
| | | (m, 1H), 8.69 (dd, J = 4.7, 1.7 Hz, | | | at 100° C. | | | |
| | | 1H), 8.55-8.41 (m, 1H), 8.26 (d, J = | | | | | | |
| | | 8.6 Hz, 1H), 8.22 (dd, J = 2.9, 1.4 | | | | | | |
| | | Hz, 1H), 8.11 (d, J = 1.7 Hz, 1H), | | | | | | |
| | | 7.93 (dd, J = 8.6, 1.8 Hz, 1H), 7.81 | | | | | | |
| | | (dd, J = 5.1, 1.4 Hz, 1H), 7.72 (dd, | | | | | | |
| | | J = 5.0, 2.9 Hz, 1H), 7.55 (ddd, | | | | | | |
| | | J = 8.0, 4.8, 0.8 Hz, 1H), 3.17 | | | | | | |
| | | (d, J = 4.5 Hz, 3H). | | | | | | |
| 321 | | 1H NMR (400 MHz, DMSO) δ 9.64 | DMSO | >98 | Method L | | | |
| | | (dd, J = 2.1, 0.8 Hz, 1H), 8.83-8.73 | | | Temperature | | | |
| | | (m, 1H), 8.69 (dd, J = 4.8, 1.7 Hz, | | | at 100° C. | | | |
| | | 1H), 8.60-8.48 (m, 1H), 8.28 (d, | | | | | | |
| | | J = 8.4 Hz, 1H), 7.68 (d, J = 1.6 Hz, | | | | | | |
| | | 1H), 7.54 (ddd, J = 8.0, 4.8, 0.8 Hz, | | | | | | |
| | | 1H), 7.50 (dd, J = 8.4, 1.8 Hz, 1H), | | | | | | |
| | | 7.38 (dd, J = 8.5, 6.1 Hz, 1H), 7.23 | | | | | | |
| | | (dd, J = 10.2, 2.6 Hz, 1H), 7.19- | | | | | | |
| | | 7.10 (m, 1H), 3.19 (d, J = 4.5 Hz, | | | | | | |
| | | 3H), 2.30 (s, 3H). | | | | | | |
| 322 | | 1H NMR (400 MHz, DMSO) δ 9.64 | DMSO | >98 | Method L | | | |
| | | (dd, J = 2.1, 0.8 Hz, 1H), 8.83-8.72 | | | Temperature | | | |
| | | (m, 1H), 8.68 (dd, J = 4.7, 1.7 Hz, 1H), | | | at 100° C. | | | |
| | | 8.56-8.43 (m, 1H), 8.23 (d, J = 8.5 | | | | | | |
| | | Hz, 1H), 7.83 (d, J = 1.7 Hz, 1H), | | | | | | |
| | | 7.61 (dd, J = 8.5, 1.8 Hz, 1H), 7.58- | | | | | | |
| | | 7.45 (m, 2H), 7.10 (dd, J = 11.5, 2.5 | | | | | | |
| | | Hz, 1H), 6.96-6.87 (m, 1H), 3.84 (s, | | | | | | |
| | | 3H), 3.18 (d, J = 4.5 Hz, 3H). | | | | | | |
| 323 | 2 HCl | 1H NMR (400 MHz, DMSO) δ 10.45 | DMSO | >98 | Method L | | | |
| | | (brs, 1H), 9.74 (d, J = 1.7 Hz, 1H), | | | Temperature | | | |
| | | 9.19 (d, J = 8.2 Hz, 1H), 8.99 (dd, | | | at 100° C. | | | |
| | | J = 5.1, 1.4 Hz, 1H), 8.71 (d, J = | | | | | | |
| | | 8.5 Hz, 1H), 8.28 (s, 1H), 7.93 | | | | | | |
| | | (dd, J = 8.0, 5.2 Hz, 1H), 7.73 (d, | | | | | | |
| | | J = 8.3 Hz, 1H), 7.42-7.34 (m, | | | | | | |
| | | 1H), 7.34-7.26 (m, 1H), 3.32 (d, | | | | | | |
| | | J = 4.5 Hz, 3H), 2.09 (d, J = 2.3 Hz, 3H). | | | | | | |
| 324 | | 1H NMR (400 MHz, DMSO) δ 9.65 | DMSO | >98 | Method L | | | |
| | | (dd, J = 2.1, 0.8 Hz, 1H), 8.82- | | | Temperature | | | |
| | | 8.75 (m, 1H), 8.69 (dd, J = 4.7, | | | at 100° C. | | | |
| | | 1.7 Hz, 1H), 8.61-8.51 (m, 1H), | | | | | | |
| | | 8.30 (d, J = 8.4 Hz, 1H), 7.71 (d, J = | | | | | | |
| | | 1.6 Hz, 1H), 7.58-7.48 (m, 2H), 7.41- | | | | | | |
| | | 7.30 (m, 1H), 7.29-7.18 (m, 2H), | | | | | | |
| | | 3.19 (d, J = 4.5 Hz, 3H), 2.20 (d, | | | | | | |
| | | J = 2.4 Hz, 3H). | | | | | | |
| 325 | | 1H NMR (400 MHz, DMSO) δ 9.58 | DMSO | >98 | Method L | | | |
| | | (dd, J = 2.1, 0.8 Hz, 1H), 8.76- | | | Temperature | | | |
| | | 8.64 (m, 2H), 8.37 (d, 1H), 8.12 | | | at 100° C. | | | |
| | | (d, J = 1.9 Hz, 1H), 8.04 (brs, 2H), | | | | | | |
| | | 7.98-7.91 (m, 2H), 7.89 (dd, J = | | | | | | |
| | | 8.6, 1.9 Hz, 1H), 7.55 (ddd, J = | | | | | | |
| | | 7.9, 4.8, 0.8 Hz, 1H). | | | | | | |
| 326 | | 1H NMR (400 MHz, DMSO) δ 9.66 | DMSO | >98 | Method L | | | |
| | | (dd, J = 2.1, 0.7 Hz, 1H), 8.83-8.75 | | | Temperature | | | |
| | | (m, 1H), 8.70 (dd, J = 4.8, 1.7 Hz, 1H), | | | at 100° C. | | | |
| | | 8.54-8.47 (m, 1H), 8.26 (d, J = 1.6 | | | | | | |
| | | Hz, 1H), 7.86 (d, J = 8.5 Hz, 1H), | | | | | | |
| | | 7.79 (dd, J = 8.5, 1.8 Hz, 1H), 7.55 | | | | | | |
| | | (ddd, J = 8.0, 4.8, 0.8 Hz, 1H), 7.43- | | | | | | |
| | | 7.32 (m, 1H), 7.30-7.18 (m, 2H), | | | | | | |
| | | 3.16 (d, J = 4.5 Hz, 3H), 2.21 (d, J = 2.3 | | | | | | |
| | | Hz, 3H). | | | | | | |
| 327 | | 1H NMR (400 MHz, DMSO) δ 10.45 | DMSO | >98 | Method L | | | |
| | | (brs, 1H), 9.74 (d, J = 1.6 Hz, 1H), | | | Temperature | | | |
| | | 9.26-9.13 (m, 1H), 8.99 (dd, J = | | | at 100° C. | | | |
| | | 5.1, 1.5 Hz, 1H), 8.65 (d, J = 1.4 | | | | | | |
| | | Hz, 1H), 8.38 (d, J = 8.6 Hz, 1H), | | | | | | |
| | | 8.06 (dd, J = 8.6, 1.7 Hz, 1H), 7.94 | | | | | | |
| | | (dd, J = 8.0, 5.2 Hz, 1H), 7.50-7.37 | | | | | | |
| | | (m, 1H), 7.30-7.13 (m, 2H), 3.29 | | | | | | |
| | | (d, J = 4.5 Hz, 3H), 2.30 (s, 3H). | | | | | | |
| 328 | 3 HCl | ¹H NMR (400 MHz, DMSO) δ 10.03 | DMSO | >98 | Method | | | |
| | | (s, 1H), 9.94 (dd, J = 2.2, 1.3 Hz, | | | I, J, K, E | | | |
| | | 1H), 9.42 (dd, J = 5.4, 1.2 Hz, 1H), | | | G1, R3 | | | |
| | | 9.00 (d, J = 1.8 Hz, 1H), 8.82 (d, | | | | | | |
| | | J = 4.9 Hz, 1H), 8.46 (dd, J = 5.3, | | | | | | |
| | | 2.2 Hz, 1H), 8.42-8.33 (m, 2H), | | | | | | |
| | | 8.04 (dd, J = 23.3, 8.4 Hz, 2H), 7.84- | | | | | | |
| | | 7.69 (m, 3H), 7.40-7.28 (m, 1H), | | | | | | |
| | | 5.31 (d, J = 5.5 Hz, 2H). | | | | | | |
| 329 | 3 HCl | ¹H NMR (400 MHz, DMSO) δ 9.93 | DMSO | >98 | Method | | | |
| | | (dd, J = 2.2, 1.3 Hz, 1H), 9.80 (t, | | | I, J, K, E | | | |
| | | J = 5.7 Hz, 1H), 9.41 (dd, J = 5.3, | | | G1, R3 | | | |
| | | 1.2 Hz, 1H), 8.79 (d, J = 4.7 Hz, | | | | | | |
| | | 1H), 8.68 (s, 1H), 8.43 (dd, J = | | | | | | |
| | | 5.3, 2.3 Hz, 1H), 8.33 (t, J = 7.9 | | | | | | |
| | | Hz, 1H), 8.10 (dd, J = 6.2, 4.3 Hz, | | | | | | |
| | | 1H), 8.06-7.96 (m, 2H), 7.85-7.72 | | | | | | |
| | | (m, 2H), 7.49 (ddd, J = 11.6, 9.3, | | | | | | |
| | | 2.6 Hz, 1H), 7.33 (td, J = 8.3, 2.3 Hz, | | | | | | |
| | | 1H), 5.25 (d, J = 5.4 Hz, 2H). | | | | | | |
| 330 | 3 HCl | ¹H NMR (400 MHz, DMSO) δ 9.94 | DMSO | >98 | Method | | | |
| | | (dd, J = 2.2, 1.3 Hz, 1H), 9.77 (s, | | | I, J, K, E | | | |
| | | 1H), 9.41 (dd, J = 5.3, 1.2 Hz, 1H), | | | G1, R3 | | | |
| | | 8.81-8.70 (m, 2H), 8.43 (dd, J = | | | | | | |
| | | 5.3, 2.3 Hz, 1H), 8.29 (t, J = 7.4 Hz, | | | | | | |
| | | 1H), 8.15 (dd, J = 6.3, 4.3 Hz, 1H), | | | | | | |
| | | 7.99 (dd, J = 19.0, 8.2 Hz, 2H), | | | | | | |
| | | 7.77-7.69 (m, 1H), 7.65 (ddd, J = | | | | | | |
| | | 9.3, 6.2, 3.2 Hz, 1H), 7.54-7.45 | | | | | | |
| | | (m, 1H), 7.38 (ddd, J = 12.4, 8.5, | | | | | | |
| | | 3.5 Hz, 1H), 5.24 (d, J = 5.5 Hz, 2H). | | | | | | |
| 331 | 3 HCl | ¹H NMR (400 MHz, DMSO) δ 9.95 | DMSO | >98 | Method | | | |
| | | (dt, J = 5.4, 2.7 Hz, 2H), 9.43 (dd, | | | I, J, K, E | | | |
| | | J = 5.4, 1.2 Hz, 1H), 8.82 (d, J = 4.8 | | | G1, L | | | |
| | | Hz, 1H), 8.74 (d, J = 1.4 Hz, 1H), | | | | | | |
| | | 8.49 (dd, J = 5.4, 2.3 Hz, 1H), 8.39 | | | | | | |
| | | (t, J = 7.7 Hz, 1H), 8.15 (dt, J = 8.6, | | | | | | |
| | | 1.9 Hz, 1H), 8.06 (t, J = 7.0 Hz, 2H), | | | | | | |
| | | 7.86-7.73 (m, 2H), 7.54 (tdd, J = 7.0, | | | | | | |
| | | 5.2, 1.8 Hz, 1H), 7.46-7.38 (m, 2H), | | | | | | |
| | | 5.30 (d, J = 5.4 Hz, 2H). | | | | | | |
| 332 | 4 HCl | ¹H NMR (400 MHz, DMSO) δ 10.46 | DMSO | >98 | Method | | | |
| | | (s, 1H), 10.01 (dd, J = 2.2, 1.2 Hz, | | | I, J, K, E | | | |
| | | 1H), 9.49-9.43 (m, 3H), 9.30-9.23 | | | G1, L | | | |
| | | (m, 2H), 8.87 (dd, J = 5.7, 0.9 Hz, | | | | | | |
| | | 1H), 8.60 (dd, J = 5.4, 2.3 Hz, 1H), | | | | | | |
| | | 8.52-8.44 (m, 2H), 8.18 (d, J = 8.1 | | | | | | |
| | | Hz, 1H), 8.12 (d, J = 8.7 Hz, 1H), | | | | | | |
| | | 7.90 (t, J = 6.7 Hz, 1H), 5.40 (t, | | | | | | |
| | | J = 14.0 Hz, 2H). | | | | | | |
| 333 | 3 HCl | ¹H NMR (400 MHz, DMSO) δ 9.96 | DMSO | >98 | Method | | | |
| | | (dd, J = 2.2, 1.3 Hz, 1H), 9.47 (t, | | | I, J, K, E | | | |
| | | J = 5.8 Hz, 1H), 9.38 (dd, J = 5.3, | | | G1, R3 | | | |
| | | 1.3 Hz, 1H), 8.69 (s, 1H), 8.56 | | | | | | |
| | | (ddd, J = 4.8, 1.7, 0.9 Hz, 1H), | | | | | | |
| | | 8.39 (dd, J = 5.3, 2.2 Hz, 1H), | | | | | | |
| | | 8.08 (dt, J = 8.6, 1.9 Hz, 1H), 7.96 | | | | | | |
| | | (d, J = 8.7 Hz, 1H), 7.75 (td, J = | | | | | | |
| | | 7.7, 1.8 Hz, 1H), 7.58-7.47 (m, | | | | | | |
| | | 3H), 7.44-7.36 (m, 1H), 7.27 | | | | | | |
| | | (ddd, J = 7.5, 4.9, 1.0 Hz, 1H), | | | | | | |
| | | 5.03 (d, J = 5.7 Hz, 2H). | | | | | | |
| 334 | 3 HCl | ¹H NMR (400 MHz, DMSO) δ 10.29 | DMSO | >98 | Method | | | |
| | | (s, 1H), 9.53 (d, J = 1.8 Hz, 1H), | | | I, J, K, E | | | |
| | | 9.12 (d, J = 8.0 Hz, 1H), 8.93 (dd, | | | G1, R3 | | | |
| | | J = 5.3, 1.4 Hz, 1H), 8.78 (d, J = | | | | | | |
| | | 2.5 Hz, 2H), 8.31 (t, J = 7.8 Hz, | | | | | | |
| | | 1H), 8.13 (q, J = 8.7 Hz, 2H), 8.04- | | | | | | |
| | | 7.90 (m, 2H), 7.87-7.70 (m, 2H), | | | | | | |
| | | 7.50 (ddd, J = 11.6, 9.3, 2.6 Hz, | | | | | | |
| | | 1H), 7.33 (td, J = 8.3, 1.9 Hz, | | | | | | |
| | | 1H), 5.30 (d, J = 5.4 Hz, 2H). | | | | | | |
| 335 | 3 HCl | ¹H NMR (400 MHz, DMSO) δ 9.91 | DMSO | >98 | Method | | | |
| | | (s, 1H), 9.64 (d, J = 1.7 Hz, 1H), | | | I, J, K, E | | | |
| | | 9.12 (d, J = 7.8 Hz, 1H), 8.97 (dd, | | | G1, R3 | | | |
| | | J = 5.2, 1.5 Hz, 1H), 8.72-8.60 | | | | | | |
| | | (m, 2H), 8.35 (t, J = 7.4 Hz, 1H), | | | | | | |
| | | 8.16 (s, 2H), 8.04-7.87 (m, 2H), | | | | | | |
| | | 7.79-7.67 (m, 2H), 7.53 (tdd, J = | | | | | | |
| | | 7.0, 5.2, 1.8 Hz, 1H), 7.41 (ddd, | | | | | | |
| | | J = 10.0, 7.3, 3.2 Hz, 2H), 4.29 | | | | | | |
| | | (dd, J = 11.9, 5.9 Hz, 2H). | | | | | | |
| 336 | 3 HCl | ¹H NMR (400 MHz, DMSO) δ 10.21 | DMSO | >98 | Method | | | |
| | | (s, 1H), 9.64 (d, J = 1.6 Hz, 1H), | | | I, J, K, E | | | |
| | | 9.12 (d, J = 7.8 Hz, 1H), 9.01-8.88 | | | G1, R3 | | | |
| | | (m, 2H), 8.69 (dd, J = 5.8, 0.9 Hz, | | | | | | |
| | | 1H), 8.42-8.32 (m, 2H), 8.18 (d, | | | | | | |
| | | J = 8.5 Hz, 1H), 8.04 (d, J = 8.0 | | | | | | |
| | | Hz, 1H), 7.94 (dd, J = 7.8, 5.2 Hz, | | | | | | |
| | | 1H), 7.84-7.70 (m, 3H), 7.60 (td, | | | | | | |
| | | J = 8.2, 6.3 Hz, 1H), 7.30 (td, J = | | | | | | |
| | | 8.3, 1.9 Hz, 1H), 4.32 (dd, J = 12.1, | | | | | | |
| | | 6.0 Hz, 2H). | | | | | | |
| 337 | 3 HCl | ¹H NMR (400 MHz, DMSO) δ 10.25 | DMSO | >98 | Method | | | |
| | | (s, 1H), 9.64 (d, J = 1.6 Hz, 1H), | | | I, J, K, E | | | |
| | | 9.11 (d, J = 7.8 Hz, 1H), 8.97 (dd, | | | G1, R3 | | | |
| | | J = 5.1, 1.5 Hz, 1H), 8.87 (s, 1H), | | | | | | |
| | | 8.69 (dd, J = 5.8, 0.9 Hz, 1H), 8.40- | | | | | | |
| | | 8.29 (m, 2H), 8.19 (d, J = 8.5 Hz, | | | | | | |
| | | 1H), 8.07-7.88 (m, 5H), 7.78-7.71 | | | | | | |
| | | (m, 1H), 7.44-7.35 (m, 2H), 4.31 | | | | | | |
| | | (dd, J = 12.1, 5.9 Hz, 2H). | | | | | | |
| 338 | 3 HCl | ¹H NMR (400 MHz, DMSO) δ 9.64 | DMSO | >98 | Method | | | |
| | | (dd, J = 2.1, 0.7 Hz, 1H), 8.77 (dt, | | | I, J, K, E | | | |
| | | J = 8.0, 1.9 Hz, 1H), 8.75-8.67 (m, | | | G1, R3 | | | |
| | | 2H), 8.53 (ddd, J = 4.8, 1.8, 0.9 Hz, | | | | | | |
| | | 1H), 8.44 (s, 1H), 7.95 (dt, J = 8.6, | | | | | | |
| | | 1.9 Hz, 1H), 7.87 (d, J = 8.6 Hz, | | | | | | |
| | | 1H), 7.76-7.66 (m, 2H), 7.56 (ddd, | | | | | | |
| | | J = 8.0, 4.8, 0.8 Hz, 1H), 7.50-7.42 | | | | | | |
| | | (m, 1H), 7.36-7.26 (m, 2H), 7.21 | | | | | | |
| | | (ddd, J = 7.5, 4.9, 1.1 Hz, 1H), | | | | | | |
| | | 4.06 (dd, J = 13.1, 6.9 Hz, 2H), | | | | | | |
| | | 3.23 (t, J = 7.3 Hz, 2H). | | | | | | |
| 339 | 3 HCl | ¹H NMR (400 MHz, DMSO) δ 9.88 | DMSO | >98 | Method | | | |
| | | (s, 1H), 9.65 (d, J = 1.7 Hz, 1H), 9.14 | | | I, J, K, E | | | |
| | | (d, J = 7.6 Hz, 1H), 8.97 (dd, J = 5.2, | | | G1, R3 | | | |
| | | 1.5 Hz, 1H), 8.68 (d, J = 3.6 Hz, 2H), | | | | | | |
| | | 8.36 (t, J = 7.2 Hz, 1H), 8.18 (d, J = | | | | | | |
| | | 8.5 Hz, 2H), 8.01 (d, J = 8.0 Hz, | | | | | | |
| | | 1H), 7.98-7.90 (m, 1H), 7.78-7.72 | | | | | | |
| | | (m, 1H), 7.64 (ddd, J = 9.2, 6.2, 3.2 | | | | | | |
| | | Hz, 1H), 7.52-7.44 (m, 1H), 7.38 | | | | | | |
| | | (ddd, J = 12.2, 8.4, 3.5 Hz, 1H), | | | | | | |
| | | 4.29 (dd, J = 12.2, 6.0 Hz, 2H), 3.60- | | | | | | |
| | | 3.48 (m, 2H). | | | | | | |
| 340 | 3 HCl | ¹H NMR (400 MHz, DMSO) δ 10.06 | DMSO | >98 | Method | | | |
| | | (s, 1H), 9.66 (d, J = 1.8 Hz, 1H), | | | I, J, K, E | | | |
| | | 9.13 (d, J = 8.1 Hz, 1H), 8.97 (dd, | | | G1, R3 | | | |
| | | J = 5.1, 1.4 Hz, 1H), 8.70 (d, J = | | | | | | |
| | | 4.9 Hz, 1H), 8.55 (d, J = 8.7 Hz, | | | | | | |
| | | 1H), 8.42 (s, 1H), 8.37 (t, J = 7.2 | | | | | | |
| | | Hz, 1H), 8.02 (dd, J = 11.7, 9.2 | | | | | | |
| | | Hz, 2H), 7.95-7.88 (m, 3H), 7.79- | | | | | | |
| | | 7.74 (m, 1H), 7.43 (dd, J = 11.1, | | | | | | |
| | | 6.6 Hz, 2H), 4.28 (d, J = 6.1 | | | | | | |
| | | Hz, 2H), 3.64-3.51 (m, 2H). | | | | | | |
| 341 | 4 HCl | ¹H NMR (400 MHz, DMSO) δ 10.36 | DMSO | >98 | Method | | | |
| | | (s, 1H), 9.53 (d, J = 1.7 Hz, 1H), 9.09 | | | I, J, K, E | | | |
| | | (d, J = 7.9 Hz, 1H), 8.93 (dd, J = 5.2, | | | G1, L | | | |
| | | 1.3 Hz, 1H), 8.83 (s, 1H), 8.78 (d, | | | | | | |
| | | J = 5.0 Hz, 1H), 8.31 (dd, J = 24.3, | | | | | | |
| | | 7.9 Hz, 3H), 8.21 (d, J = 8.5 Hz, | | | | | | |
| | | 1H), 7.99 (d, J = 8.2 Hz, 1H), 7.95- | | | | | | |
| | | 7.87 (m, 1H), 7.79-7.70 (m, 1H), | | | | | | |
| | | 7.57 (dt, J = 7.2, 3.5 Hz, 1H), 7.50 | | | | | | |
| | | (d, J = 3.2 Hz, 1H), 7.38-7.32 (m, | | | | | | |
| | | 2H), 6.69 (d, J = 3.1 Hz, 1H), 5.32 | | | | | | |
| | | (d, J = 5.4 Hz, 2H), 3.89 (s, 3H). | | | | | | |
| 342 | 3 HCl | ¹H NMR (400 MHz, DMSO) δ 10.55 | DMSO | >98 | Method | | | |
| | | (s, 1H), 9.52 (d, J = 1.7 Hz, 1H), | | | I, J, K, E | | | |
| | | 9.07 (d, J = 8.2 Hz, 1H), 8.98- | | | G1, L | | | |
| | | 8.88 (m, 2H), 8.78 (d, J = 4.8 Hz, | | | | | | |
| | | 1H), 8.46-8.38 (m, 1H), 8.29 (t, | | | | | | |
| | | J = 7.2 Hz, 1H), 8.15 (dd, J = | | | | | | |
| | | 13.0, 5.1 Hz, 2H), 8.00 (d, J = 7.7 | | | | | | |
| | | Hz, 1H), 7.93-7.85 (m, 1H), 7.80- | | | | | | |
| | | 7.70 (m, 2H), 7.62 (d, J = 8.6 | | | | | | |
| | | Hz, 1H), 7.43 (d, J = 3.1 Hz, 1H), | | | | | | |
| | | 6.55 (dd, J = 3.1, 0.7 Hz, 1H), | | | | | | |
| | | 5.34 (d, J = 5.5 Hz, 2H). | | | | | | |
| 343 | 3 HCl | ¹H NMR (400 MHz, DMSO) δ 10.23 | DMSO | >98 | Method | | | |
| | | (s, 1H), 9.50 (s, 1H), 9.13 (s, 1H), | | | I, J, K, E | | | |
| | | 9.02 (d, J = 7.9 Hz, 1H), 8.89 (d, J = | | | G1, L | | | |
| | | 5.1 Hz, 1H), 8.76 (d, J = 4.9 Hz, | | | | | | |
| | | 1H), 8.48 (d, J = 8.7 Hz, 1H), 8.24 | | | | | | |
| | | (s, 1H), 8.07 (d, J = 8.7 Hz, 1H), | | | | | | |
| | | 7.95 (d, J = 7.7 Hz, 1H), 7.92-7.83 (m, | | | | | | |
| | | 1H), 7.76 (d, J = 7.5 Hz, 1H), 7.68 (d, | | | | | | |
| | | J = 8.1 Hz, 2H), 7.40 (t, J = 7.8 Hz, | | | | | | |
| | | 1H), 7.33 (t, J = 7.3 Hz, 1H), 5.28 | | | | | | |
| | | (d, J = 5.5 Hz, 2H). | | | | | | |
| 344 | 3 HCl | ¹H NMR (400 MHz, DMSO) δ 10.77 | DMSO | >98 | Method | | | |
| | | (s, 1H), 9.56 (d, J = 1.7 Hz, 1H), | | | I, J, K, E | | | |
| | | 9.18 (d, J = 8.2 Hz, 1H), 9.00 (d, J = | | | G1, L | | | |
| | | 1.6 Hz, 1H), 8.94 (dd, J = 5.3, | | | | | | |
| | | 1.5 Hz, 1H), 8.83 (dd, J = 5.6, 0.8 | | | | | | |
| | | Hz, 1H), 8.44-8.31 (m, 2H), 8.15 | | | | | | |
| | | (dd, J = 24.2, 8.4 Hz, 2H), 7.98- | | | | | | |
| | | 7.91 (m, 3H), 7.87-7.80 (m, 1H), | | | | | | |
| | | 7.15-7.07 (m, 2H), 5.39 (d, J = | | | | | | |
| | | 5.5 Hz, 2H), 3.95 (s, 3H). | | | | | | |
| 345 | 3 HCl | ¹H NMR (400 MHz, DMSO) δ 10.11 | DMSO | >98 | Method | | | |
| | | (s, 1H), 9.65 (d, J = 1.6 Hz, 1H), | | | I, J, K, E | | | |
| | | 9.17 (d, J = 8.2 Hz, 1H), 9.09 (s, | | | G1, L | | | |
| | | 1H), 8.97 (dd, J = 6.3, 5.0 Hz, | | | | | | |
| | | 2H), 8.71 (d, J = 4.9 Hz, 1H), 8.46 | | | | | | |
| | | (d, J = 8.7 Hz, 1H), 8.39 (t, J = | | | | | | |
| | | 7.8 Hz, 1H), 8.16 (d, J = 8.4 Hz, | | | | | | |
| | | 1H), 8.05 (d, J = 8.0 Hz, 1H), 7.99- | | | | | | |
| | | 7.92 (m, 1H), 7.72 (ddd, J = | | | | | | |
| | | 27.6, 14.6, 7.4 Hz, 5H), 7.39 (dd, | | | | | | |
| | | J = 11.2, 4.1 Hz, 1H), 7.32 (t, J = | | | | | | |
| | | 7.4 Hz, 1H), 4.32 (d, J = 5.8 Hz, | | | | | | |
| | | 2H), 3.60 (t, J = 6.2 Hz, 2H). | | | | | | |
| 346 | 3 HCl | ¹H NMR (400 MHz, DMSO)δ 10.19 | DMSO | >98 | Method | | | |
| | | (s, 1H), 9.62 (d, J = 1.7 Hz, 1H), | | | I, J, K, E | | | |
| | | 9.04 (s, 1H), 8.95 (dd, J = 5.1, 1.5 | | | G1, L | | | |
| | | Hz, 1H), 8.80 (s, 1H), 8.69 (d, J = | | | | | | |
| | | 4.9 Hz, 1H), 8.43-8.30 (m, 2H), | | | | | | |
| | | 8.21 (s, 1H), 8.08 (d, J = 1.4 Hz, | | | | | | |
| | | 1H), 8.01 (d, J = 7.9 Hz, 1H), 7.88 | | | | | | |
| | | (s, 1H), 7.78-7.68 (m, 2H), 7.61 | | | | | | |
| | | (d, J = 8.6 Hz, 1H), 7.43 (d, J = | | | | | | |
| | | 3.0 Hz, 1H), 6.54 (dd, J = 3.1, 0.6 | | | | | | |
| | | Hz, 1H), 4.37-4.24 (m, 2H), 3.85 | | | | | | |
| | | (s, 3H), 3.57 (t, J = 6.2 Hz, 2H). | | | | | | |
| 347 | 3 HCl | ¹H NMR (400 MHz, DMSO) δ 10.46 | DMSO | >98 | Method | | | |
| | | (s, 1H), 9.55 (d, J = 1.8 Hz, 1H), | | | I, J, K, E | | | |
| | | 9.19 (d, J = 8.0 Hz, 1H), 8.95 (dd, | | | G1, L | | | |
| | | J = 5.4, 1.4 Hz, 1H), 8.86-8.82 | | | | | | |
| | | (m, 1H), 8.73 (d, J = 8.8 Hz, 1H), | | | | | | |
| | | 8.50 (s, 1H), 8.41 (t, J = 7.9 Hz, | | | | | | |
| | | 1H), 8.28 (dd, J = 8.7, 1.7 Hz, | | | | | | |
| | | 1H), 8.08 (t, J = 7.8 Hz, 1H), 7.98 | | | | | | |
| | | (dd, J = 8.1, 5.4 Hz, 1H), 7.86- | | | | | | |
| | | 7.81 (m, 2H), 7.79-7.69 (m, 2H), | | | | | | |
| | | 7.46-7.39 (m, 1H), 7.37-7.30 | | | | | | |
| | | (m, 1H), 5.33 (dd, J = 16.7, 5.4 | | | | | | |
| | | Hz, 2H). | | | | | | |
| 348 | 3 HCl | ¹H NMR (400 MHz, DMSO) δ 10.47 | DMSO | >98 | Method | | | |
| | | (s, 1H), 9.53 (d, J = 1.8 Hz, 1H), | | | I, J, K, E | | | |
| | | 9.15 (d, J = 8.0 Hz, 1H), 9.00 (d, J = | | | G1, L | | | |
| | | 1.4 Hz, 1H), 8.93 (dd, J = 5.3, 1.4 | | | | | | |
| | | Hz, 1H), 8.81 (d, J = 4.8 Hz, 1H), | | | | | | |
| | | 8.43-8.26 (m, 2H), 8.08 (t, J = 8.2 | | | | | | |
| | | Hz, 2H), 8.00-7.89 (m, 2H), 7.85- | | | | | | |
| | | 7.77 (m, 1H), 7.27 (d, J = 3.3 Hz, | | | | | | |
| | | 1H), 6.73 (dd, J = 3.4, 1.8 Hz, 1H), | | | | | | |
| | | 5.34 (d, J = 5.5 Hz, 2H) | | | | | | |
| 601 | 3 HCl | ¹H NMR (400 MHz, DMSO) δ 10.68 | DMSO | >98 | Method | | | |
| | | (s, 1H), 9.55 (s, 1H), 9.20 (s, 1H), | | | I, J, K, E | | | |
| | | 9.03 (s, 1H), 8.95 (d, J = 5.3 Hz, | | | G1, L | | | |
| | | 1H), 8.84 (d, J = 5.5 Hz, 1H), 8.43 | | | | | | |
| | | (s, 1H), 8.28 (dd, J = 8.7, 1.8 Hz, | | | | | | |
| | | 1H), 8.11 (t, J = 9.2 Hz, 2H), 8.02- | | | | | | |
| | | 7.94 (m, 1H), 7.91-7.79 (m, | | | | | | |
| | | 2H), 7.71 (dd, J = 5.1, 1.0 Hz, | | | | | | |
| | | 1H), 7.25 (dd, J = 5.0, 3.7 Hz, | | | | | | |
| | | 1H), 5.38 (d, J = 3.8 Hz, 2H). | | | | | | |
| 602 | 3 HCl | ¹H NMR (400 MHz, DMSO) δ 10.63 | DMSO | >98 | Method | | | |
| | | (s, 1H), 9.51 (d, J = 1.7 Hz, 1H), | | | I, J, K, E | | | |
| | | 9.13-9.01 (m, 2H), 8.92 (dd, J = | | | G1, L | | | |
| | | 5.3, 1.5 Hz, 1H), 8.80 (d, J = 4.8 | | | | | | |
| | | Hz, 1H), 8.47-8.43 (m, 1H), 8.38- | | | | | | |
| | | 8.25 (m, 2H), 8.06 (d, J = 8.7 | | | | | | |
| | | Hz, 2H), 7.97-7.85 (m, 2H), 7.82- | | | | | | |
| | | 7.74 (m, 1H), 7.28 (dd, J = 1.8, | | | | | | |
| | | 0.8 Hz, 1H), 5.33 (d, J = 5.5 Hz, 2H). | | | | | | |
| 603 | 2 HCl | ¹H NMR (400 MHz, DMSO) δ 10.00 | DMSO | >98 | Method | | | |
| | | (s, 1H), 9.58 (d, J = 1.6 Hz, 1H), | | | I, J, K, E | | | |
| | | 9.06 (d, J = 8.3 Hz, 1H), 8.99- | | | G1, L | | | |
| | | 8.89 (m, 2H), 8.18 (s, 2H), 7.97- | | | | | | |
| | | 7.88 (m, 1H), 7.79-7.72 (m, 1H), | | | | | | |
| | | 7.62-7.51 (m, 3H), 7.47-7.33 | | | | | | |
| | | (m, 4H), 7.25 (t, J = 7.4 Hz, 1H), | | | | | | |
| | | 5.91 (p, J = 6.9 Hz, 1H), | | | | | | |
| | | 1.72 (d, J = 7.0 Hz, 3H). | | | | | | |
| 604 | 2 HCl | ¹H NMR (400 MHz, DMSO) δ 10.03 | DMSO | >98 | Method | | | |
| | | (s, 1H), 9.56 (d, J = 1.6 Hz, 1H), | | | I, J, K, E | | | |
| | | 9.11-9.00 (m, 2H), 8.97-8.89 (m, | | | G1, L | | | |
| | | 1H), 8.38 (dd, J = 8.8, 1.6 Hz, 1H), | | | | | | |
| | | 8.15 (d, J = 8.8 Hz, 1H), 7.94-7.86 | | | | | | |
| | | (m, 1H), 7.86-7.76 (m, 2H), 7.63 (dd, | | | | | | |
| | | J = 13.3, 7.7 Hz, 3H), 7.41-7.21 (m, | | | | | | |
| | | 4H), 6.01-5.78 (m, 1H), 1.76 (d, | | | | | | |
| | | J = 7.0 Hz, 3H). | | | | | | |
| 605 | 2 HCl | ¹H NMR (400 MHz, DMSO) δ 10.10- | DMSO | >98 | Method | | | |
| | | 9.94 (m, 1H), 9.55 (d, J = 1.7 Hz, | | | I, J, K, E | | | |
| | | 1H), 9.01 (d, J = 14.3 Hz, 2H), | | | G1, L | | | |
| | | 8.93 (dd, J = 5.1, 1.4 Hz, 1H), | | | | | | |
| | | 8.33 (d, J = 8.8 Hz, 1H), | | | | | | |
| | | 8.15 (d, J = 8.9 Hz, 1H), | | | | | | |
| | | 8.03-7.96 (m, 2H), 7.93-7.83 | | | | | | |
| | | (m, 1H), 7.61 (d, J = 7.3 Hz, 2H), | | | | | | |
| | | 7.40 (dt, J = 15.3, 8.3 Hz, 4H), | | | | | | |
| | | 7.26 (t, J = 7.4 Hz, 1H), 6.03-5.83 | | | | | | |
| | | (m, 1H), 1.75 (d, J = 7.0 Hz, 3H). | | | | | | |
| 606 | 2 HCl | ¹H NMR (400 MHz, DMSO) δ 10.05 | DMSO | >98 | Method | | | |
| | | (s, 1H), 9.59 (dd, J = 4.9, 1.9 Hz, | | | I, J, K, E | | | |
| | | 1H), 9.12 (d, J = 8.2 Hz, 1H), 9.01- | | | G1, L | | | |
| | | 8.92 (m, 2H), 8.27-8.16 (m, 2H), | | | | | | |
| | | 7.96 (dd, J = 8.0, 5.3 Hz, 1H), 7.63- | | | | | | |
| | | 7.53 (m, 4H), 7.46-7.32 (m, 3H), | | | | | | |
| | | 7.29-7.21 (m, 1H), 5.91 (p, J = 7.0 | | | | | | |
| | | Hz, 1H), 1.73 (d, J = 7.0 Hz, 3H). | | | | | | |
| 607 | 2 HCl | ¹H NMR (400 MHz, DMSO) δ 9.92 | DMSO | >98 | Method | | | |
| | | (s, 1H), 9.56 (d, J = 1.7 Hz, 1H), | | | I, J, K, E | | | |
| | | 9.05 (d, J = 8.0 Hz, 1H), 8.95 (dd, | | | G1, L | | | |
| | | J = 5.2, 1.4 Hz, 1H), 8.89 (s, 1H), | | | | | | |
| | | 8.14 (s, 2H), 7.93 (dd, J = 7.9, 5.3 | | | | | | |
| | | Hz, 1H), 7.81 (td, J = 8.9, 6.6 Hz, | | | | | | |
| | | 1H), 7.59 (d, J = 7.2 Hz, 2H), 7.51 | | | | | | |
| | | (ddd, J = 11.6, 9.3, 2.5 Hz, 1H), | | | | | | |
| | | 7.40-7.30 (m, 3H), 7.25 (t, J = | | | | | | |
| | | 7.4 Hz, 1H), 5.89 (dd, J = 14.2, | | | | | | |
| | | 7.1 Hz, 1H), 1.72 (d, J = 7.0 Hz, 3H). | | | | | | |
| 608 | 2 HCl | ¹H NMR (400 MHz, DMSO) δ 9.91 | DMSO | >98 | Method | | | |
| | | (s, 1H), 9.57 (d, J = 1.7 Hz, 1H), | | | I, J, K, E | | | |
| | | 9.07 (d, J = 8.0 Hz, 1H), 8.99- | | | G1, L | | | |
| | | 8.90 (m, 2H), 8.17 (q, J = 8.6 Hz, | | | | | | |
| | | 2H), 7.94 (dd, J = 7.9, 5.3 Hz, | | | | | | |
| | | 1H), 7.68 (ddd, J = 9.1, 6.2, 3.2 | | | | | | |
| | | Hz, 1H), 7.60 (d, J = 7.3 Hz, 3H), | | | | | | |
| | | 7.50 (td, J = 9.6, 4.6 Hz, 1H), 7.44- | | | | | | |
| | | 7.33 (m, 3H), 7.25 (t, J = 7.4 | | | | | | |
| | | Hz, 1H), 5.90 (t, J = 7.1. Hz, 1H), | | | | | | |
| | | 1.71 (t, J = 12.9 Hz, 3H). | | | | | | |
| 349 | 2 HCl | ¹H NMR (400 MHz, DMSO) δ 9.98 | DMSO | >98 | Method | | | |
| | | (s, 1H), 9.56 (d, J = 1.8 Hz, 1H), | | | I, J, K, E | | | |
| | | 9.14-9.00 (m, 2H), 8.94 (dd, J = | | | G1, L | | | |
| | | 5.2, 1.4 Hz, 1H), 8.39 (dd, J = 8.8, | | | | | | |
| | | 1.7 Hz, 1H), 8.12 (d, J = 8.8 Hz, | | | | | | |
| | | 1H), 7.96-7.88 (m, 1H), 7.80- | | | | | | |
| | | 7.71 (m, 2H), 7.62 (d, J = 7.3 Hz, | | | | | | |
| | | 2H), 7.41-7.32 (m, 3H), 7.26 (t, | | | | | | |
| | | J = 7.4 Hz, 1H), 5.91 (t, J = 7.2 | | | | | | |
| | | Hz, 1H), 1.76 (d, J = 7.0 Hz, 3H). | | | | | | |
| 350 | 2 HCl | ¹H NMR (400 MHz, DMSO) δ 10.03 | DMSO | >98 | Method | | | |
| | | (s, 1H), 9.55 (d, J = 1.6 Hz, 1H), | | | I, J, K, E | | | |
| | | 9.09-8.98 (m, 2H), 8.96-8.90 | | | G1, L | | | |
| | | (m, 1H), 8.33 (dd, J = 8.8, 1.7 Hz, | | | | | | |
| | | 1H), 8.17-8.05 (m, 2H), 7.90 | | | | | | |
| | | (dd, J = 7.8, 5.3 Hz, 1H), 7.85- | | | | | | |
| | | 7.79 (m, 1H), 7.65 (ddd, J = 11.6, | | | | | | |
| | | 9.5, 7.9 Hz, 3H), 7.38 (dd, J = | | | | | | |
| | | 10.5, 4.8 Hz, 2H), 7.26 (t, J = 7.4 | | | | | | |
| | | Hz, 1H), 5.91 (p, J = 6.9 Hz, 1H), | | | | | | |
| | | 1.76 (d, J = 7.0 Hz, 4H). | | | | | | |
| 351 | 3 HCl | ¹H NMR (400 MHz, DMSO) δ 10.29 | DMSO | >98 | Method | | | |
| | | (s, 1H), 9.53 (d, J = 1.8 Hz, 1H), | | | I, J, K, E | | | |
| | | 9.12 (d, J = 8.0 Hz, 1H), 8.93 (dd, | | | G1, L | | | |
| | | J = 5.3, 1.4 Hz, 1H), 8.82-8.74 | | | | | | |
| | | (m, 2H), 8.31 (t, J = 7.8 Hz, 1H), | | | | | | |
| | | 8.19-8.09 (m, 2H), 7.99 (d, J = | | | | | | |
| | | 8.0 Hz, 1H), 7.94 (dd, J = 8.1, 5.4 | | | | | | |
| | | Hz, 1H), 7.82 (td, J = 8.9, 6.6 Hz, | | | | | | |
| | | 1H), 7.78-7.72 (m, 1H), 7.54- | | | | | | |
| | | 7.45 (m, 1H), 7.33 (td, J = 8.3, | | | | | | |
| | | 1.9 Hz, 1H), 5.30 (d, J = 5.4 Hz, 2H). | | | | | | |
| 352 | 3 HCl | ¹H NMR (400 MHz, DMSO) δ 10.62 | DMSO | >98 | Method | | | |
| | | (s, 1H), 9.56 (d, J = 1.7 Hz, 1H), | | | I, J, K, E | | | |
| | | 9.24-9.12 (m, 2H), 8.94 (dd, J = | | | G1, L | | | |
| | | 5.3, 1.3 Hz, 1H), 8.81 (d, J = 5.0 | | | | | | |
| | | Hz, 1H), 8.48-8.45 (m, 1H), 8.43 | | | | | | |
| | | (dd, J = 8.8, 1.9 Hz, 1H), 8.35 | | | | | | |
| | | (dd, J = 8.0, 1.0 Hz, 2H), 8.16 (d, | | | | | | |
| | | J = 8.8 Hz, 1H), 8.08 (d, J = 8.2 | | | | | | |
| | | Hz, 1H), 7.99-7.91 (m, 2H), 7.83- | | | | | | |
| | | 7.75 (m, 2H), 5.37 (d, J = 5.5 Hz, 2H). | | | | | | |
| 353 | 4 HCl | ¹H NMR (400 MHz, DMSO) δ 11.27 | DMSO | >98 | Method | | | |
| | | (s, 1H), 9.48 (d, J = 1.9 Hz, 1H), | | | I, J, K, E | | | |
| | | 9.34 (t, J = 5.8 Hz, 1H), 8.75 (d, J = | | | G1, L | | | |
| | | 1.6 Hz, 1H), 8.71-8.60 (m, 2H), 8.56 | | | | | | |
| | | (d, J = 4.0 Hz, 1H), 8.20 (dd, J = 8.7, | | | | | | |
| | | 1.8 Hz, 1H), 7.97-7.82 (m, 2H), | | | | | | |
| | | 7.81-7.65 (m, 2H), 7.62-7.38 (m, | | | | | | |
| | | 4H), 7.26 (dd, J = 6.8, 5.3 Hz, 1H), | | | | | | |
| | | 6.50 (s, 1H), 5.03 (d, J = 5.6 Hz, 2H). | | | | | | |
| 354 | 3 HCl | ¹H NMR (400 MHz, DMSO) δ 10.30 | DMSO | >98 | Method | | | |
| | | (s, 1H), 9.55 (d, J = 1.7 Hz, 1H), 9.15 | | | I, J, K, E | | | |
| | | (d,J = 7.6 Hz, 1H),8.98-8.92 (m, | | | G1, L | | | |
| | | 1H), 8.85-8.76 (m, 2H), 8.34 (t, J = | | | | | | |
| | | 7.6 Hz, 1H), 8.26-8.18 (m, 2H), | | | | | | |
| | | 8.15 (dd, J = 7.1, 1.3 Hz, 1H), 8.03 | | | | | | |
| | | (d, J = 8.0 Hz, 1H), 8.00-7.93 (m, | | | | | | |
| | | 1H), 7.92 (d, J = 5.6 Hz, 1H), 7.81- | | | | | | |
| | | 7.74 (m, 1H), 7.61-7.54 (m, 3H), | | | | | | |
| | | 5.32 (d, J = 5.3 Hz, 2H). | | | | | | |
| 355 | 3 HCl | ¹H NMR (400 MHz, DMSO) δ 11.44 | DMSO | >98 | Method | | | |
| | | (s, 1H), 10.27 (s, 1H), 9.50 (d, J = | | | I, J, K, E | | | |
| | | 1.6 Hz, 1H), 8.99 (d, J = 7.0 Hz, | | | G1, L | | | |
| | | 1H), 8.90 (dd, J = 5.2, 1.5 Hz, | | | | | | |
| | | 1H), 8.82 (d, J = 1.4 Hz, 1H), 8.74 | | | | | | |
| | | (d, J = 5.0 Hz, 1H), 8.35 (dd, J = | | | | | | |
| | | 8.6, 1.6 Hz, 1H), 8.18 (t, J = 8.7 | | | | | | |
| | | Hz, 2H), 7.95-7.83 (m, 2H), 7.70- | | | | | | |
| | | 7.61 (m, 1H), 7.56-7.48 (m, | | | | | | |
| | | 2H), 7.35-7.25 (m, 2H), 6.72- | | | | | | |
| | | 6.66 (m, 1H), 5.27 (d, J = 5.4 Hz, 2H). | | | | | | |
| 356 | 3 HCl | ¹H NMR (400 MHz, DMSO) δ 10.57 | DMSO | >98 | Method | | | |
| | | (s, 1H), 9.53 (d, J = 1.6 Hz, 1H), | | | I, J, K, E | | | |
| | | 9.10 (d, J = 8.1 Hz, 1H), 9.02 (d, J = | | | G1, L | | | |
| | | 1.5 Hz, 1H), 8.92 (dd, J = 5.3, 1.5 Hz, | | | | | | |
| | | 1H), 8.80 (d, J = 4.7 Hz, 1H), 8.43 | | | | | | |
| | | (dd, J = 8.8, 1.8 Hz, 1H), 8.36-8.28 | | | | | | |
| | | (m, 2H), 8.20-8.13 (m, 2H), 8.06- | | | | | | |
| | | 8.00 (m, 2H), 7.92 (dd, J = 8.1, | | | | | | |
| | | 5.3 Hz, 1H), 7.84 (d, J = 8.9 Hz, | | | | | | |
| | | 1H), 7.80-7.73 (m, 1H), 5.35 (d, | | | | | | |
| | | J = 5.5 Hz, 2H), 4.11 (s, 3H). | | | | | | |
| 357 | 3 HCl | ¹H NMR (400 MHz, DMSO) δ 10.41 | DMSO | >98 | Method | | | |
| | | (s, 1H), 9.53 (d, J = 1.8 Hz, 1H), | | | I, J, K, E | | | |
| | | 9.14-9.05 (m, 2H), 8.91 (dd, J = | | | G1, L | | | |
| | | 5.3, 1.4 Hz, 1H), 8.77 (d, J = 5.4 | | | | | | |
| | | Hz, 1H), 8.41 (dd, J = 8.8, 1.9 Hz, | | | | | | |
| | | 1H), 8.28 (t, J = 7.8 Hz, 1H), 8.17 | | | | | | |
| | | (d, J = 8.5 Hz, 2H), 8.12 (d, J = | | | | | | |
| | | 8.7 Hz, 1H), 8.05 (d, J = 8.6 Hz, | | | | | | |
| | | 2H), 7.99 (d, J = 8.0 Hz, 1H), 7.92 | | | | | | |
| | | (dd, J = 8.1, 5.3 Hz, 1H), 7.73 (t, | | | | | | |
| | | J = 6.5 Hz, 1H), 5.31 (d, J = 5.5 Hz, 2H). | | | | | | |
| 358 | 3 HCl | ¹H NMR (400 MHz, DMSO) δ 10.28 | DMSO | >98 | Method | | | |
| | | (s, 1H), 9.54 (d, J = 1.8 Hz, 1H), | | | I, J, K, E | | | |
| | | 9.11 (d, J = 7.4 Hz, 1H), 8.93 (dd, | | | G1, L | | | |
| | | J = 5.3, 1.4 Hz, 1H), 8.85 (d, J = | | | | | | |
| | | 1.2 Hz, 1H), 8.78 (d, J = 5.0 Hz, | | | | | | |
| | | 1H), 8.34-8.23 (m, 2H), 8.22- | | | | | | |
| | | 8.13 (m, 2H), 8.10-8.03 (m, 2H), | | | | | | |
| | | 7.99 (d, J = 7.7 Hz, 1H), 7.94 (dd, | | | | | | |
| | | J = 7.8, 5.4 Hz, 1H), 7.77-7.70 | | | | | | |
| | | (m, 1H), 7.57-7.47 (m, 2H), 5.31 | | | | | | |
| | | (d, J = 5.5 Hz, 2H). | | | | | | |
| 359 | 3 HCl | ¹H NMR (400 MHz, DMSO) δ 10.29 | DMSO | >98 | Method | | | |
| | | (s, 1H), 9.53 (d, J = 1.8 Hz, 1H), | | | I, J, K, E | | | |
| | | 9.12 (d, J = 8.0 Hz, 1H), 8.93 (dd, | | | G1, L | | | |
| | | J = 5.3, 1.4 Hz, 1H), 8.82-8.74 | | | | | | |
| | | (m, 2H), 8.31 (t, J = 7.8 Hz, 1H), | | | | | | |
| | | 8.19-8.09 (m, 2H), 7.99 (d, J = | | | | | | |
| | | 8.0 Hz, 1H), 7.94 (dd, J = 8.1, 5.4 | | | | | | |
| | | Hz, 1H), 7.82 (td, J = 8.9, 6.6 Hz, | | | | | | |
| | | 1H), 7.78-7.72 (m, 1H), 7.54- | | | | | | |
| | | 7.45 (m, 1H), 7.33 (td, J = 8.3, | | | | | | |
| | | 1.9 Hz, 1H), 5.30 (d, J = 5.4 Hz, 2H). | | | | | | |
| 360 | 3 HCl | ¹H NMR (400 MHz, DMSO) δ 10.42 | DMSO | >98 | Method | | | |
| | | (s, 1H), 9.55 (d, J = 1.7 Hz, 1H), | | | I, J, K, E | | | |
| | | 9.14 (d, J = 7.6 Hz, 1H), 8.94 (dd, | | | G1, L | | | |
| | | J = 5.3, 1.4 Hz, 1H), 8.80 (d, J = | | | | | | |
| | | 4.7 Hz, 1H), 8.68 (s, 1H), 8.35 (t, | | | | | | |
| | | J = 7.9 Hz, 1H), 8.20-8.08 (m, | | | | | | |
| | | 2H), 8.03 (d, J = 8.1 Hz, 1H), 7.94 | | | | | | |
| | | (dd, J = 7.8, 5.4 Hz, 1H), 7.83- | | | | | | |
| | | 7.75 (m, 1H), 7.52 (dd, J = 7.5, | | | | | | |
| | | 1.7 Hz, 1H), 7.50-7.42 (m, 1H), | | | | | | |
| | | 7.22 (d, J = 7.7 Hz, 1H), 7.14 (td, | | | | | | |
| | | J = 7.5, 1.0 Hz, 1H), 5.34 (d, J = | | | | | | |
| | | 5.5 Hz, 2H), 3.84 (s, 3H). | | | | | | |
| 361 | 2 HCl | ¹H NMR (400 MHz, DMSO) δ 10.09 | DMSO | >98 | Method | | | |
| | | (s, 1H), 9.61 (d, J = 1.6 Hz, 1H), | | | I, J, K, E | | | |
| | | 9.08 (d, J = 7.2 Hz, 1H), 8.93 (dd, | | | G1, L | | | |
| | | J = 5.2, 1.5 Hz, 1H), 8.76 (s, 1H), | | | | | | |
| | | 8.18 (d, J = 8.7 Hz, 1H), 8.11 | | | | | | |
| | | (d, J = 8.7 Hz, 1H), 7.92 (dd, | | | | | | |
| | | J = 7.6, 5.4 Hz, 1H), 7.64-7.51 | | | | | | |
| | | (m, 3H), 7.48-7.33 (m, 3H), 5.01 | | | | | | |
| | | (d, J = 5.6 Hz, 2H). | | | | | | |
| 362 | 2 HCl | ¹H NMR (400 MHz, DMSO) δ 9.89- | DMSO | >98 | Method | | | |
| | | 9.41 (m, 3H), 9.14-8.83 (m, | | | I, J, K, E | | | |
| | | 3H), 8.79-8.66 (m, 1H), 8.31- | | | G1, L | | | |
| | | 7.81 (m, 5H), 7.73-7.51 (m, 3H), | | | | | | |
| | | 7.49-7.37 (m, 1H), 6.09-5.89 | | | | | | |
| | | (m, 1H), 1.87-1.71 (m, 3H). | | | | | | |
| 363 | 2 HCl | ¹H NMR (400 MHz, DMSO) δ 9.87- | DMSO | >98 | Method | | | |
| | | 9.46 (m, 2H), 9.10-8.83 (m, | | | I, J, K, E | | | |
| | | 3H), 8.79-8.66 (m, 1H), 8.24- | | | G1, L | | | |
| | | 7.78 (m, 6H), 7.66-7.45 (m, 2H), | | | | | | |
| | | 7.43-7.30 (m, 1H), 6.07-5.89 | | | | | | |
| | | (m, 1H), 1.79 (d, J = 6.8 Hz, 3H). | | | | | | |
| 364 | 2 HCl | ¹H NMR (400 MHz, DMSO)δ 9.90- | DMSO | >98 | Method | | | |
| | | 9.35 (m, 2H), 9.15-8.83 (m, 3H), | | | I, J, K, E | | | |
| | | 8.78-8.63 (m, 1H), 8.33-7.34 (m, | | | G1, L | | | |
| | | 9H), 6.09-5.84 (m, 1H), 1.87- | | | | | | |
| | | 1.72 (m, 3H). | | | | | | |
| 365 | 2 HCl | ¹H NMR (400 MHz, DMSO) δ 9.91 | DMSO | >98 | Method | | | |
| | | (s, 1H), 9.51 (s, 1H), 9.22-8.98 | | | I, J, K, E | | | |
| | | (m, 2H), 8.95-8.86 (m, 1H), 8.81- | | | G1, L | | | |
| | | 8.70 (m, 1H), 8.43-8.33 (m, 1H), | | | | | | |
| | | 8.29-7.75 (m, 6H), 7.71-7.55 (m, 1H), | | | | | | |
| | | 7.44-7.31 (m, 1H),6.12-5.95 (m, | | | | | | |
| | | 1H), 1.85 (d, J = 7.0 Hz, 3H). | | | | | | |
| 366 | 2 HCl | ¹H NMR (400 MHz, DMSO) δ 9.83 | DMSO | >98 | Method | | | |
| | | (s, 1H), 9.55-9.44 (m, 1H), 9.15- | | | I, J, K, E | | | |
| | | 8.85 (m, 3H), 8.77-8.65 (m, 1H), | | | G1, L | | | |
| | | 8.29-7.71 (m, 6H), 7.69-7.50 (m, | | | | | | |
| | | 2H), 7.48-7.36 (m, 2H), 6.08-5.91 | | | | | | |
| | | (m, 1H), 1.80 (d, J = 6.9 Hz, 3H). | | | | | | |
| 367 | 2 HCl | ¹H NMR (400 MHz, DMSO) δ 10.10 | DMSO | >98 | Method | | | |
| | | (s, 1H), 9.54 (s, 1H), 9.37-8.72 (m, | | | I, J, K, E | | | |
| | | 4H), 8.51-7.56 (m, 9H), 7.43-7.25 (m, | | | G1, L | | | |
| | | 1H), 6.26-5.95 (m, 1H), 1.87 (d, | | | | | | |
| | | J = 6.7 Hz, 3H). | | | | | | |
| 368 | 2 HCl | ¹H NMR (400 MHz, DMSO) δ 9.81 | DMSO | >98 | Method | | | |
| | | (s, 1H), 9.58-9.48 (m, 1H), 9.20- | | | I, J, K, E | | | |
| | | 9.02 (m, 1H), 8.99-8.72 (m, | | | G1, L | | | |
| | | 3H), 8.40-8.15 (m, 2H), 8.12- | | | | | | |
| | | 7.87 (m, 3H), 7.78-7.62 (m, 2H), | | | | | | |
| | | 7.57-7.36 (m, 2H), 6.14-5.94 | | | | | | |
| | | (m, 1H), 1.89-1.76 (m, 3H). | | | | | | |
| 369 | 2 HCl | ¹H NMR (400 MHz, DMSO) δ 9.68 | DMSO | >98 | Method | | | |
| | | (s, 1H), 9.52-9.41 (m, 1H), 9.10- | | | I, J, K, E | | | |
| | | 8.63 (m, 4H), 8.34-7.74 (m, | | | G1, L | | | |
| | | 7H), 7.71-7.48 (m, 2H), 6.04- | | | | | | |
| | | 5.82 (m, 1H), 1.80 (d, J = 6.8 Hz, 3H). | | | | | | |
| 370 | 2 HCl | ¹H NMR (400 MHz, DMSO) δ 9.75 | DMSO | >98 | Method | | | |
| | | (s, 1H), 9.53 (s, 1H), 9.23-9.09 | | | I, J, K, E | | | |
| | | (m, 1H), 8.99-8.92 (m, 1H), 8.90- | | | G1, L | | | |
| | | 8.82 (m, 1H), 8.82-8.74 (m, 1H), | | | | | | |
| | | 8.35 (s, 2H), 8.19-8.04 (m, 2H), 8.04- | | | | | | |
| | | 7.94 (m, 1H), 7.81-7.67 (m, 3H), | | | | | | |
| | | 7.45-7.31 (m, 1H), 6.12-5.99 (m, | | | | | | |
| | | 1H), 1.83 (d, J = 7.0 Hz, 3H). | | | | | | |
| 371 | 2 HCl | ¹H NMR (400 MHz, DMSO) δ 9.96- | DMSO | >98 | Method | | | |
| | | 9.63 (m, 1H), 9.50 (s, 1H), 9.15- | | | I, J, K, E | | | |
| | | 8.63 (m, 4H), 8.30-7.49 (m, | | | G1, L | | | |
| | | 8H), 7.48-7.36 (m, 2H), 6.10- | | | | | | |
| | | 5.93 (m, 1H), 1.85-1.73 (m, 3H). | | | | | | |
| 372 | 2 HCl | ¹H NMR (400 MHz, DMSO) δ 9.71 | DMSO | >98 | Method | | | |
| | | (s, 1H), 9.49 (s, 1H), 9.07-8.96 | | | I, J, K, E | | | |
| | | (m, 1H), 8.96-8.87 (m, 1H), 8.87- | | | G1, L | | | |
| | | 8.78 (m, 1H), 8.78-8.69 (m, 1H), | | | | | | |
| | | 8.31 (s, 1H), 8.25-8.09 (m, 2H), 8.01- | | | | | | |
| | | 7.84 (m, 2H), 7.82-7.72 (m, 2H), | | | | | | |
| | | 7.68-7.56 (m, 2H), 7.41-7.29 (m, | | | | | | |
| | | 1H), 6.05-5.89 (m, 1H), 1.80 (d, | | | | | | |
| | | J = 7.1 Hz, 3H). | | | | | | |
| 373 | 3 HCl | ¹H NMR (400 MHz, DMSO) δ 10.60 | DMSO | >98 | Method | | | |
| | | (s, 1H), 9.55 (d, J = 1.7 Hz, 1H), | | | I, J, K, E | | | |
| | | 9.23-9.05 (m, 2H), 8.94 (dd, J = | | | G1, L | | | |
| | | 5.3, 1.4 Hz, 1H), 8.81 (d, J = 4.8 | | | | | | |
| | | Hz, 1H), 8.46-8.31 (m, 2H), 8.15 | | | | | | |
| | | (d, J = 8.8 Hz, 1H), 8.06 (d, J = | | | | | | |
| | | 8.0 Hz, 1H), 8.02-7.76 (m, 4H), | | | | | | |
| | | 7.67-7.57 (m, 1H), 7.38-7.26 | | | | | | |
| | | (m, 1H), 5.36 (d, J = 5.5 Hz, 2H). | | | | | | |
| 374 | 3 HCl | ¹H NMR (400 MHz, DMSO) δ 10.67 | DMSO | >98 | Method | | | |
| | | (s, 1H), 9.56 (d, J = 1.7Hz, 1H), | | | I, J, K, E | | | |
| | | 9.18 (d, J = 7.9 Hz, 1H), 9.04 (s, | | | G1, L | | | |
| | | 1H), 8.94 (dd, J = 5.3, 1.4 Hz, | | | | | | |
| | | 1H), 8.82 (d, J = 4.8 Hz, 1H), 8.44- | | | | | | |
| | | 8.31 (m, 2H), 8.22-7.91 (m, | | | | | | |
| | | 5H), 7.86-7.77 (m, 1H), 7.49- | | | | | | |
| | | 7.37 (m, 2H), 5.37 (d, J = 5.5 Hz, 2H). | | | | | | |
| 375 | 3 HCl | ¹H NMR (400 MHz, DMSO) δ 10.57 | DMSO | >98 | Method | | | |
| | | (s, 1H), 9.54 (d, J = 1.9 Hz, 1H), | | | I, J, K, E | | | |
| | | 9.21-9.02 (m, 2H), 8.93 (dd, J = 5.3, | | | G1, L | | | |
| | | 1.4 Hz, 1H), 8.80 (d, J = 5.4 Hz, 1H), | | | | | | |
| | | 8.46-8.29 (m, 2H), 8.19-8.01 (m, | | | | | | |
| | | 3H), 8.01-7.74 (m, 3H), 7.72-7.59 | | | | | | |
| | | (m, 1H), 5.34 (d, J = 5.4 Hz, 2H). | | | | | | |
| 376 | 3 HCl | ¹H NMR (400 MHz, DMSO) δ 10.62 | DMSO | >98 | Method | | | |
| | | (s, 1H), 9.56 (d, J = 1.8 Hz, 1H), | | | I, J, K, E | | | |
| | | 9.31-9.09 (m, 2H), 8.94 (dd, J = | | | G1, L | | | |
| | | 5.3, 1.4 Hz, 1H), 8.82 (d, J = 4.7 | | | | | | |
| | | Hz, 1H), 8.54-8.34 (m, 2H), 8.11 | | | | | | |
| | | (dd, J = 12.1, 8.5 Hz, 2H), 7.96 | | | | | | |
| | | (dd, J = 8.1, 5.4 Hz, 1H), 7.91- | | | | | | |
| | | 7.75 (m, 3H), 7.46-7.28 (m, 1H), | | | | | | |
| | | 5.37 (d, J = 5.6 Hz, 2H). | | | | | | |
| 377 | 3 HCl | ¹H NMR (400 MHz, DMSO) δ 10.55 | DMSO | >98 | Method | | | |
| | | (s, 1H), 9.54 (d, J = 1.7 Hz, 1H), | | | I, J, K, E | | | |
| | | 9.12 (d, J = 7.4 Hz, 1H), 9.01 (s, | | | G1, L | | | |
| | | H), 8.93 (dd, J = 5.3, 1.4 Hz, | | | | | | |
| | | 1H), 8.79 (d, J = 5.3 Hz, 1H), 8.45- | | | | | | |
| | | 8.26 (m, 2H), 8.16 (d, J = 8.9 | | | | | | |
| | | Hz, 1H), 8.10-7.86 (m, 4H), 7.84- | | | | | | |
| | | 7.70 (m, 1H), 7.63-7.53 (m, 2H), | | | | | | |
| | | 7.53-7.38 (m, 1H), 5.34 (d, | | | | | | |
| | | J = 5.5 Hz, 2H). | | | | | | |
| 378 | 3 HCl | ¹H NMR (400 MHz, DMSO) δ 10.60 | DMSO | >98 | Method | | | |
| | | (s, 1H), 9.58 (d, J = 1.7 Hz, 1H), | | | I, J, K, E | | | |
| | | 9.23 (d, J = 8.1 Hz, 1H), 8.96 (dd, | | | G1, L | | | |
| | | J = 5.3, 1.4 Hz, 1H), 8.83 (dd, J = | | | | | | |
| | | 5.6, 0.8 Hz, 1H), 8.67 (d, J = 1.3 | | | | | | |
| | | Hz, 1H), 8.47-8.38 (m, 1H), 8.22 | | | | | | |
| | | (d, J = 8.4 Hz, 1H), 8.15-7.92 (m, 3H), | | | | | | |
| | | 7.92-7.80 (m, 1H), 7.46- 7.29 (m, 4H), | | | | | | |
| | | 5.36 (d, J = 5.5 Hz, 2H), 2.37 (s, 3H). | | | | | | |
| 379 | 3 HCl | ¹H NMR (400 MHz, DMSO) δ 10.67 | DMSO | >98 | Method | | | |
| | | 1H), 9.55 (d, J = 1.4 Hz, 1H), 9.25- | | | I, J, K, E | | | |
| | | 9.10 (m, 1H), 9.01 (s, 1H), 8.93 (dd, | | | G1, L | | | |
| | | J = 5.2, 1.3 Hz, 1H), 8.81 (d, J = 5.4 | | | | | | |
| | | Hz, 1H), 8.46-8.30 (m, 2H), 8.25- | | | | | | |
| | | 8.13 (m, 1H), 8.13-8.02 (m, 1H), | | | | | | |
| | | 8.00-7.89 (m, 1H), 7.87-7.72 (m, | | | | | | |
| | | 3H), 7.45 (t, J = 7.7 Hz, 1H), 7.28 | | | | | | |
| | | (d, J = 7.5 Hz, 1H), 5.37 (d, J = 5.0 | | | | | | |
| | | Hz, 2H), 2.44 (s, 3H). | | | | | | |
| 380 | 3 HCl | ¹H NMR (400 MHz, DMSO) δ 10.46 | DMSO | >98 | Method | | | |
| | | (s, 1H), 9.51 (d, J = 1.7 Hz, 1H), | | | I, J, K, E | | | |
| | | 9.04 (m, J = 7.7 Hz, 1H), 9.00- | | | G1, L | | | |
| | | 8.87 (m, 2H), 8.76 (d, J = 4.7 Hz, | | | | | | |
| | | 1H), 8.41-8.30 (m, 1H), 8.30- | | | | | | |
| | | 8.19 (m, 1H), 8.12 (d, J = 8.8 Hz, | | | | | | |
| | | 1H), 8.04-7.82 (m, 4H), 7.76-7.62 | | | | | | |
| | | (m, 1H), 7.38 (d, J = 8.0 Hz, 2H), 5.30 | | | | | | |
| | | (d, J = 5.5 Hz, 2H), 2.40 (s, 3H). | | | | | | |
| 381 | 3 HCl | ¹H NMR (400 MHz, DMSO) δ 10.31 | DMSO | >98 | Method | | | |
| | | (s, 1H), 9.54 (d, J = 1.6 Hz, 1H), | | | I, J, K, E | | | |
| | | 9.15 (d, J = 8.2 Hz, 1H), 8.94 (dd, | | | G1, L | | | |
| | | J = 5.3, 1.3 Hz, 1H), 8.80 (d, J = | | | | | | |
| | | 5.0 Hz, 1H), 8.69 (d, J = 8.6 Hz, | | | | | | |
| | | 1H), 8.49-8.28 (m, 2H), 8.24-7.88 | | | | | | |
| | | (m, 4H), 7.88-7.74 (m, 2H), 7.74-7.54 | | | | | | |
| | | (m, 1H), 5.31 (d, J = 5.4 Hz, 2H). | | | | | | |
| 382 | 3 HCl | ¹H NMR (400 MHz, DMSO) δ 10.15 | DMSO | >98 | Method | | | |
| | | (s, 1H), 9.53 (d, J = 1.8 Hz, 1H), | | | I, J, K, E | | | |
| | | 9.11 (d, J = 7.8 Hz, 1H), 8.93 (d, J = | | | G1, L | | | |
| | | 5.2 Hz, 1H), 8.78 (d, J = 5.0 Hz, | | | | | | |
| | | 1H), 8.66 (d, J = 8.6 Hz, 1H), 8.46- | | | | | | |
| | | 8.24 (m, 2H), 8.14 (dd, J = 8.7, | | | | | | |
| | | 1.8 Hz, 1H), 8.06-7.89 (m, 2H), | | | | | | |
| | | 7.82-7.65 (m, 3H), 7.51-7.34 | | | | | | |
| | | (m, 1H), 5.28 (d, J = 5.3 Hz, 2H). | | | | | | |
| 383 | 3 HCl | ¹H NMR (400 MHz, DMSO) δ 10.42 | DMSO | >98 | Method | | | |
| | | (m, 1H), 8.93 (d, J = 4.0 Hz, 19. H), | | | I, J, K, E | | | |
| | | 8.78 (d, J = 5.4 Hz, 1H), 8.69 (d, J = | | | G1, L | | | |
| | | 8.5 Hz, 1H), 8.44-8.23 (m, | | | | | | |
| | | 2H), 8.12 (dd, J = 8.7, 1.7 Hz, | | | | | | |
| | | 1H), 8.02-7.86 (m, 4H), 7.78- | | | | | | |
| | | 7.69 (m, 1H), 7.64-7.47 (m, 3H), | | | | | | |
| | | 5.31 (d, J = 5.1 Hz, 2H). | | | | | | |
| 384 | 3 HCl | ¹H NMR (400 MHz, DMSO) δ 10.77 | DMSO | >98 | Method | | | |
| | | (s, 1H), 9.58 (d, J = 1.7 Hz, 1H), | | | I, J, K, E | | | |
| | | 9.20 (d, J = 8.1 Hz, 1H), 8.95 (dd, | | | G1, L | | | |
| | | J = 5.2, 1.2 Hz, 1H), 8.84 (d, J = | | | | | | |
| | | 5.0 Hz, 1H), 8.72 (d, J = 8.6 Hz, | | | | | | |
| | | 1H), 8.42 (t, J = 7.6 Hz, 1H), 8.31 | | | | | | |
| | | (s, 1H), 8.10 (d, J = 8.0 Hz, 1H), | | | | | | |
| | | 8.02-7.79 (m, 3H), 7.55-7.43 | | | | | | |
| | | (m, 2H), 7.23 (d, J = 8.5 Hz, 1H), | | | | | | |
| | | 7.14 (t, J = 7.4 Hz, 1H), 5.39 (d, J = | | | | | | |
| | | 5.5 Hz, 2H), 3.84 (s, 3H). | | | | | | |
| 385 | | 1H NMR (400 MHz, DMSO) δ 9.64 | DMSO | >98 | Method | | | |
| | | (d, J = 1.5 Hz, 1H), 8.83-8.73 (m, | | | I, J, K, E | | | |
| | | 1H), 8.73-8.66 (m, 2H), 8.55-8.49 | | | G1, L | | | |
| | | (m, 1H), 8.49-8.44 (m, 1H), 8.16 | | | (100° C.) | | | |
| | | (dd, J = 8.7, 1.9 Hz, 1H), 7.86 (d, | | | | | | |
| | | J = 8.7 Hz, 1H), 7.75-7.66 (m, 2H), | | | | | | |
| | | 7.64-7.52 (m, 3H), 7.52-7.46 (m, | | | | | | |
| | | 1H), 7.28-7.21 (m, 1H), 7.11 (ddd, | | | | | | |
| | | J = 7.4, 4.8, 0.9 Hz, 1H), 4.30 (d, | | | | | | |
| | | J = 5.2 Hz, 2H), 1.24 (s, 4H). | | | | | | |
| 386 | | 1H NMR (400 MHz, DMSO) δ 9.70- | DMSO | >98 | Method | | | |
| | | 9.54 (m, 1H), 8.77 (m, 1H), 8.69 | | | I, J, K, E | | | |
| | | (dd, J = 4.7, 1.7 Hz, 1H), 8.65 (d, | | | G1, L | | | |
| | | J = 1.8 Hz, 1H), 8.55-8.49 (m, | | | (100° C.) | | | |
| | | 1H), 8.49-8.44 (m, 1H), 8.13 (dd, J = | | | | | | |
| | | 8.7, 1.9 Hz, 1H), 7.86 (d, J = 8.7 Hz, | | | | | | |
| | | 1H), 7.64-7.58 (m, 1H), 7.56 (dd, | | | | | | |
| | | J = 7.9, 4.8 Hz, 1H), 7.52-7.48 | | | | | | |
| | | (m, 1H), 7.48-7.33 (m, 3H), 7.11 | | | | | | |
| | | (ddd, J = 7.4, 4.8, 1.0 Hz, 1H), 7.04- | | | | | | |
| | | 6.96 (m, 1H), 4.29 (d, J = 5.3 Hz, | | | | | | |
| | | 2H), 3.86 (s, 3H), 1.23 (s, 4H). | | | | | | |
| 387 | HCl | 1H NMR (400 MHz, DMSO) δ 9.99 | DMSO | >98 | Method | | | |
| | | (brs, 1H), 9.64 (s, 1H), 9.20 (d, J = | | | I, J, K, E | | | |
| | | 7.6 Hz, 1H), 9.06-8.91 (m, 2H), 8.47 | | | G1, L | | | |
| | | (dd, J = 5.7, 1.3 Hz, 1H), 8.36-8.32 | | | (100° C.) | | | |
| | | (m, 1H), 8.23-8.10 (m, 2H), 8.07-7.92 | | | | | | |
| | | (m, 2H), 7.90-7.76 (m, 2H), 7.65-7.57 | | | | | | |
| | | (m, 1H), 7.57-7.51 (m, 1H), 7.35- | | | | | | |
| | | 7.26 (m, 1H), 4.26 (d, J = 6.2 | | | | | | |
| | | Hz, 2H), 1.73 (s, 6H). | | | | | | |
| 388 | HCl | 1H NMR (400 MHz, DMSO) δ 10.05 | DMSO | >98 | Method | | | |
| | | (brs, 1H), 9.63 (s, 1H), 9.17 (d, J = | | | I, J, K, E | | | |
| | | 7.6 Hz, 1H), 8.99 (d, J = 4.7 Hz, | | | G1, L | | | |
| | | 1H), 8.94 (s, 1H), 8.53-8.42 (m, | | | (100° C.) | | | |
| | | 1H), 8.38-8.26 (m, 1H), 8.26- | | | | | | |
| | | 8.10 (m, 2H), 7.98 (d, J = 7.8 Hz, | | | | | | |
| | | 2H), 7.59-7.51 (m, 1H), 7.51- | | | | | | |
| | | 7.41 (m, 3H), 7.10-6.97 (m, 1H), | | | | | | |
| | | 4.26 (d, J = 6.2 Hz, 2H), 3.89 (s, | | | | | | |
| | | 3H), 1.71 (s, 6H). | | | | | | |
| 389 | | 1H NMR (400 MHz, DMSO) δ 9.40 | DMSO | >98 | Method | | | |
| | | (s, 1H), 9.29 (d, J = 1.5 Hz, 1H), | | | I, J, K, E | | | |
| | | 8.81 (d, J = 1.7 Hz, 1H), 8.59 (dd, | | | G1, L | | | |
| | | J = 4.7, 1.7 Hz, 1H), 8.54-8.51 | | | (100° C.) | | | |
| | | (m, 1H), 8.51-8.46 (m, 1H), 8.22 | | | | | | |
| | | (dd, J = 8.7, 1.9 Hz, 1H), 7.90 (d, | | | | | | |
| | | J = 8.7 Hz, 1H), 7.62-7.55 (m, | | | | | | |
| | | 1H), 7.52-7.37 (m, 5H), 7.18- | | | | | | |
| | | 7.09 (m, 1H), 7.08-6.98 (m, 1H), | | | | | | |
| | | 3.89 (s, 3H), 1.89-1.74 (m, 2H), | | | | | | |
| | | 1.55-1.37 (m, 2H). | | | | | | |
| 390 | | 1H NMR (400 MHz, DMSO) δ 9.68- | DMSO | >98 | Method | | | |
| | | 9.62 (m, 1H), 8.82-8.76 (m, | | | I, J, K, E | | | |
| | | 1H), 8.69 (dd, J = 4.7, 1.7 Hz, | | | G1, R4 | | | |
| | | 1H), 8.56 (s, 1H), 8.33 (d, J = 8.6 | | | | | | |
| | | Hz, 1H), 8.00-7.93 (m, 1H), 7.76- | | | | | | |
| | | 7.69 (m, 1H), 7.64 (ddd, J = | | | | | | |
| | | 9.2, 6.1, 3.2 Hz, 1H), 7.55 (ddd, J = | | | | | | |
| | | 8.0, 4.8, 0.7 Hz, 1H), 7.51- | | | | | | |
| | | 7.41 (m, 1H), 7.41-7.31 (m, 1H). | | | | | | |
| 391 | HCl | 1H NMR (400 MHz, DMSO) δ 10.93 | DMSO | >98 | R4 | | | |
| | | (brs, 1H), 9.84 (d, J = 1.4 Hz, 1H), | | | Temperature | | | |
| | | 9.07 (d, J = 2.4 Hz, 1H), 9.01 (dd, | | | at 95° C. | | | |
| | | J = 2.4, 1.5 Hz, 1H), 8.80 (d, J = | | | | | | |
| | | 8.7 Hz, 1H), 8.47 (s, 1H), 7.99 (d, | | | | | | |
| | | J = 8.6 Hz, 1H), 7.80-7.67 (m, | | | | | | |
| | | 1H), 7.67-7.53 (m, 1H), 7.53- | | | | | | |
| | | 7.37 (m, 2H), 3.38 (d, J = 4.6 Hz, 3H). | | | | | | |
| 392 | HCl | 1H NMR (400 MHz, DMSO) δ 11.03- | DMSO | >98 | R4 | | | |
| | | 10.85 (m, 1H), 9.81 (d, J = 1.1 | | | Temperature | | | |
| | | Hz, 1H), 9.07 (d, J = 2.4 Hz, 1H), | | | at 95° C. | | | |
| | | 9.04-8.95 (m, 1H), 8.77 (d, J = | | | | | | |
| | | 8.7 Hz, 1H), 8.52 (d, J = 1.3 Hz, | | | | | | |
| | | 1H), 8.14 (dd, J = 8.6, 1.4 Hz, | | | | | | |
| | | 1H), 7.73-7.55 (m, 3H), 7.46- | | | | | | |
| | | 7.28 (m, 1H), 3.36 (d, J = 4.6 Hz, 3H). | | | | | | |
| 393 | HCl | 1H NMR (400 MHz, DMSO) δ 10.66 | DMSO | >98 | R4 | | | |
| | | (brs, 1H), 9.84 (d, J = 1.3 Hz, 1H), | | | Temperature | | | |
| | | 9.07 (d, J = 2.3 Hz, 1H), 9.01 (dd, | | | at 95° C. | | | |
| | | J = 2.3, 1.5 Hz, 1H), 8.69 (d, J = | | | | | | |
| | | 8.7 Hz, 1H), 8.49 (d, J = 1.6 Hz, | | | | | | |
| | | 1H), 8.13 (dd, J = 8.6, 1.7 Hz, | | | | | | |
| | | 1H), 7.93-7.83 (m, 2H), 7.51- | | | | | | |
| | | 7.37 (m, 2H), 3.38 (s, 3H). | | | | | | |
| 394 | HCl | 1H NMR (400 MHz, DMSO) δ 10.90 | DMSO | >98 | R4 | | | |
| | | (brs, 1H), 9.84 (d, J = 1.4 Hz, 1H), | | | Temperature | | | |
| | | 9.06 (d, J = 2.4 Hz, 1H), 9.00 (dd, | | | at 95° C. | | | |
| | | J = 2.4, 1.5 Hz, 1H), 8.81 (d, J = | | | | | | |
| | | 8.7 Hz, 1H), 8.46 (s, 1H), 8.00 (d, | | | | | | |
| | | J = 8.6 Hz, 1H), 7.70-7.57 (m, | | | | | | |
| | | 1H), 7.57-7.49 (m, 1H), 7.49- | | | | | | |
| | | 7.37 (m, 1H), 3.37 (d, J = 4.6 Hz, 3H). | | | | | | |
| 395 | HCl | 1H NMR (400 MHz, DMSO) δ 10.89 | DMSO | >98 | R4 | | | |
| | | (s, 1H), 9.83 (d, J = 1.4 Hz, 1H), | | | Temperature | | | |
| | | 9.07 (d, J = 2.4 Hz, 1H), 9.00 (dd, | | | at 95° C. | | | |
| | | J = 2.4, 1.5 Hz, 1H), 8.78 (d, J = | | | | | | |
| | | 8.7 Hz, 1H), 8.43 (s, 1H), 7.97 (d, | | | | | | |
| | | J = 8.6 Hz, 1H), 7.87-7.72 (m, | | | | | | |
| | | 1H), 7.53 (ddd, J = 11.6, 9.3, 2.5 | | | | | | |
| | | Hz, 1H), 7.40-7.27 (m, 1H), 3.37 | | | | | | |
| | | (d, J = 4.6 Hz, 3H). | | | | | | |
| 396 | HCl | 1H NMR (400 MHz, DMSO) δ 10.86 | DMSO | >98 | R4 | | | |
| | | (s, 1H), 9.84 (d, J = 1.3 Hz, 1H), | | | Temperature | | | |
| | | 9.07 (d, J = 2.4 Hz, 1H), 9.01 (dd, | | | at 95° C. | | | |
| | | J = 2.4, 1.5 Hz, 1H), 8.78 (d, J = | | | | | | |
| | | 8.7 Hz, 1H), 8.48 (s, 1H), 8.01 (d, | | | | | | |
| | | J = 8.7 Hz, 1H), 7.64 (ddd, J = | | | | | | |
| | | 9.2, 6.1, 3.2 Hz, 1H), 7.58-7.48 | | | | | | |
| | | (m, 1H), 7.48-7.38 (m, 1H), 3.37 | | | | | | |
| | | (d, J = 4.6 Hz, 3H). | | | | | | |
| 397 | HCl | 1H NMR (400 MHz, DMSO) δ 10.84 | DMSO | >98 | R4 | | | |
| | | (s, 1H), 9.81 (d, J = 1.4 Hz, 1H), | | | Temperature | | | |
| | | 9.06 (d, J = 2.4 Hz, 1H), 9.00 (dd, | | | at 95° C. | | | |
| | | J = 2.4, 1.5 Hz, 1H), 8.77 (d, J = | | | | | | |
| | | 8.6 Hz, 1H), 8.49 (d, J = 1.6 Hz, | | | | | | |
| | | 1H), 8.13 (dd, J = 8.7, 1.7 Hz, | | | | | | |
| | | 1H), 8.00-7.90 (m, 1H), 7.76- | | | | | | |
| | | 7.61 (m, 2H), 3.36 (d, J = 4.6 Hz, 3H). | | | | | | |
| 398 | HCl | 1H NMR (400 MHz, DMSO) δ 10.91 | DMSO | >98 | R4 | | | |
| | | (brs, 1H), 9.80 (d, J = 1.3 Hz, 1H), | | | Temperature | | | |
| | | 9.04 (d, J = 2.4 Hz, 1H), 8.99 (dd, | | | at 95° C. | | | |
| | | J = 2.3, 1.5 Hz, 1H), 8.82 (d, J = | | | | | | |
| | | 8.7 Hz, 1H), 8.51 (d, J = 1.6 Hz, | | | | | | |
| | | 1H), 8.15 (dd, J = 8.7, 1.7 Hz, | | | | | | |
| | | 1H), 7.64-7.53 (m, 2H), 7.48- | | | | | | |
| | | 7.33 (m, 1H), 3.35 (d, J = 4.6 Hz, 3H). | | | | | | |
| 399 | HCl | ¹H-NMR (400 MHz, MeOD): δ 9.68 | MeOD | 95 | Method | 345.1 | t = 1.828 min | Method B |
| | | (s, 1H), 9.17 (d, J = 8.2 Hz, 1H), | | | G1 without | (M + 1) | | (NH4—HCO3) |
| | | 9.05 (d, J = 4.7 Hz, 1H), 8.77 (d, J = | | | 0.1N HCl | | | |
| | | 1.5 Hz, 1H), 8.39 (dd, J = 8.7, | | | | | | |
| | | 1.5 Hz, 1H), 8.13 (d, J = 8.7 Hz, | | | | | | |
| | | 1H), 8.08 (dd, J = 8.0, 5.4 Hz, | | | | | | |
| | | 1H), 7.70 (d, J = 7.8 Hz, 1H), 7.64 | | | | | | |
| | | (d, J = 10.2 Hz, 1H), 7.58 (dd, J = | | | | | | |
| | | 14.0, 7.9 Hz, 1H), 7.23 (td, J = | | | | | | |
| | | 8.4, 2.1 Hz, 1H), 4.04 (q, J = 7.3 | | | | | | |
| | | Hz, 2H), 1.51 (t, J = 7.3 Hz, 3H). | | | | | | |
| 400 | | ¹H-NMR (400 MHz, MeOD): δ 9.51 | MeOD | 95 | Method | 371.1 | t = 1.922 min | Method B |
| | | 1H), 8.92 (d, J = 4.8 Hz, 1H), | | | G1 without | (M + 1) | | (NH4—HCO3) |
| | | 8.83 (d, J = 8.0 Hz, 1H), 8.66 (d, J = | | | 0.1N HCl | | | |
| | | 1.6 Hz, 1H), 8.35 (dd, J = 2.0, | | | | | | |
| | | 1.6 Hz, 1H), 8.06 (d, J = 8.8 Hz, | | | | | | |
| | | 1H), 7.81 (dd, J = 4.8, 5.2 Hz, 1H), | | | | | | |
| | | 7.63-7.58 (m, 3H), 7.23 (t, J = 17.2 | | | | | | |
| | | Hz, 1H), 4.37 (s, 4H), 2.24 (s, 4H). | | | | | | |
| 401 | | ¹H-NMR (400 MHz, MeOD): δ 9.52 | MeOD | 95 | Method | 399.1 | t = 2.008 min | Method B |
| | | (s, 1H), 8.83 (d, J = 8.0 Hz, 2H), | | | G1 without | (M + 1) | | (NH4—HCO3) |
| | | 8.74 (s, 1H), 8.29 (d, J = 8.8 Hz, | | | 0.1N HCl | | | |
| | | 1H), 7.98 (d, J = 9.2 Hz, 1H), 7.77 | | | | | | |
| | | (t, J = 12.8 Hz, 1H), 7.69-7.63 | | | | | | |
| | | (m, 2H), 7.60-7.54 (m, 1H), 7.21 | | | | | | |
| | | (t, J = 17.2 Hz, 1H), 4.60 (s, 1H), | | | | | | |
| | | 2.23-2.20 (m, 2H), 1.97-1.94 | | | | | | |
| | | (m, 2H), 1.84-1.80 (m, 1H), 1.63- | | | | | | |
| | | 1.55 (m, 4H), 1.40-1.30 (m, 1H). | | | | | | |
| 402 | | ¹H-NMR (400 MHz, DMSO-d₆): δ | DMSO | 95 | Method | 363.0 | t = 1.739 min | Method B |
| | | 9.63 (s, 1H), 9.39 (s, 1H), 8.95 (d, | | | G1 without | (M + 1) | | (NH4—HCO3) |
| | | J = 8.4 Hz, 1H), 8.87-8.81 (m, | | | 0.1N HCl | | | |
| | | 2H), 8.32 (d, J = 8.8 Hz, 1H), 7.96 | | | | | | |
| | | (d, J = 8.4 Hz, 1H), 7.77-7.75 (m, | | | | | | |
| | | 3H), 7.64-7.59 (m,, 1H), 7.30 (t, J = | | | | | | |
| | | 18.0 Hz, 1H), 4.87 (t, J = 9.2 Hz, | | | | | | |
| | | 1H), 4.75 (t, J = 9.2 Hz, 1H), 4.16- | | | | | | |
| | | 4.15 (m, 1H), 4.09-4.08 (m, 1H). | | | | | | |
| 403 | | ¹H-NMR(400 MHz, DMSO-d₆): δ | DMSO | 95 | Method | 375.1 | t = 1.641 min | Method B |
| | | 9.59(s, 1H), 8.88-8.87 (m, 2H), | | | G1 without | (M + 1) | | (NH4—HCO3) |
| | | 8.78 (s, 1H), 8.32 (d, J = 8.4 Hz, | | | 0.1N HCl | | | |
| | | 1H), 7.96-7.94 (m, 1H), 7.76- | | | | | | |
| | | 7.74 (m, 3H), 7.64-7.59 (m, 1H), | | | | | | |
| | | 7.31 (t, J = 17.6 Hz, 1H), 3.87- | | | | | | |
| | | 3.86 (m, 2H), 3.60 (t, J = 12.4 Hz, | | | | | | |
| | | 2H), 1.97-1.91 (m, 2H). | | | | | | |
| 404 | HCl | ¹H-NMR (400 MHz, DMSO-d₆): δ | DMSO | 94 | Method G4 | 433.0 | t = 1.850 min | Method B |
| | | 9.69 (d, J = 1.4 Hz, 1H), 9.29 (s, | | | | (M + 1) | | (NH4—HCO3) |
| | | 1H), 9.15 (d, J = 8.1 Hz, 1H), 8.98- | | | | | | |
| | | 8.90 (m, 1H), 8.62 (s, 1H), 7.92 | | | | | | |
| | | (t, J = 6.6 Hz, 2H), 7.64-7.62 (m, | | | | | | |
| | | 1H), 7.50-7.35 (m, 2H), 3.19 | | | | | | |
| | | (d, J = 4.4 Hz, 3H). | | | | | | |
| 405 | | ¹H-NMR (400 MHz, DMSO-d₆); δ | DMSO | 95 | Method G4 | 388.9 | t = 1.649 min | Method A |
| | | 9.63 (d, J = 1.4 Hz, 1H), 8.83- | | | | (M + 1) | | (CF3—COOH) |
| | | 8.74 (m, 1H), 8.73-8.60 (m, 3H), | | | | | | |
| | | 8.19 (dd, J = 8.7, 1.8 Hz, 1H), 7.86 | | | | | | |
| | | (d, J = 8.7 Hz, 1H), 7.74 (d, J = 8.0 | | | | | | |
| | | Hz, 2H), 7.64-7.51 (m, 2H), 7.27 | | | | | | |
| | | (dt, J = 7.8, 1.6 Hz, 1H), 3.78 (dd, | | | | | | |
| | | J = 12.5, 6.6 Hz, 2H), 3.50 (t, J = | | | | | | |
| | | 6.2 Hz, 2H), 3.28 (s, 3H), 2.10- | | | | | | |
| | | 1.96 (m, 2H). | | | | | | |
| 406 | HCl | ₁H-NMR (400 MHz, MeOD): δ 9.85 | MeOD | 98 | Method | 380.8 | t = 1.451 min | Method B |
| | | (s, 1H), 9.50 (d, J = 8.1 Hz, 1H), | | | G1 without | (M + 1) | | (NH4—HCO3) |
| | | 9.18 (d, J = 5.5 Hz, 1H), 8.75 (s, | | | 0.1N HCl | | | |
| | | 1H), 8.40-8.31 (m, 2H), 8.23 (d, J = | | | | | | |
| | | 8.7 Hz, 1H), 7.50 (t, J = 7.0 Hz, | | | | | | |
| | | 1H), 7.47-7.32 (m, 2H), 4.92 (s, | | | | | | |
| | | 1H), 4.79 (t, J = 4.8 Hz, 1H), 4.37 | | | | | | |
| | | (t, J = 4.8 Hz, 1H), 4.30 (t, J = 4.8 | | | | | | |
| | | Hz, 1H). | | | | | | |
| 407 | | ¹H-NMR (400 MHz, DMSO-d₆): δ | DMSO | 99 | Method | 385.1 | t = 2.055 min | Method B |
| | | 9.61 (d, J = 1.6 Hz, 1H), 8.75 (dt, J = | | | G1 without | (M + 1) | | (NH4—HCO3) |
| | | 7.8, 1.8 Hz, 1H), 8.70 (dd, J = | | | 0.1N HCl | | | |
| | | 4.8, 1.6 Hz, 1H), 8.18-8.15 (m, | | | | | | |
| | | 2H), 7.95 (d, J = 8.8 Hz, 1H), 7.68- | | | | | | |
| | | 7.64 (m, 2H), 7.60-7.54 (m, | | | | | | |
| | | 2H), 7.26 (td, J = 8.5, 1.7 Hz, 1H), | | | | | | |
| | | 3.91 (s, 4H), 1.76 (s, 6H). | | | | | | |
| 408 | | ¹H-NMR (400 MHz, DMSO-d₆): δ | DMSO | 100 | Method | 432.2 | t = 1.790 min | Method B |
| | | 9.61 (d, J = 1.6 Hz, 1H), 8.76 (dt, J = | | | G1 without | (M + 1) | | (NH4—HCO3) |
| | | 8.0, 1.6 Hz, 1H), 8.68 (dd, J = | | | 0.1N HCl | | | |
| | | 4.8, 1.6 Hz, 1H), 8.42 (d, J = 8.8 | | | | | | |
| | | Hz, 1H), 7.99 (s, 1H), 7.67-7.61 | | | | | | |
| | | (m, 2H), 7.54 (dd, J = 8.2, 4.6 Hz, | | | | | | |
| | | 1H), 7.46 (td, J = 9.5, 4.4 Hz, 1H), | | | | | | |
| | | 7.39-7.34 (m, 1H), 4.21-4.12 | | | | | | |
| | | (m, 2H), 4.06-4.00 (m, 1H), 3.84 | | | | | | |
| | | (t, J = 9.6 Hz, 1H), 2.88-2.81 (m, | | | | | | |
| | | 1H), 2.27 (s, 6H), 2.25-2.18 (m, | | | | | | |
| | | 1H), 1.95-1.85 (m, 1H). | | | | | | |
| 409 | | ¹H-NMR (400 MHz, MeOD): δ 9.56 | MeOD | 98 | Method | 375.1 | t = 1.74 min | Method B |
| | | (s, 1H), 8.82(s, 1H), 8.62 (s, 1H), | | | G1 without | (M + 1) | | (NH4—HCO3) |
| | | 8.40 (s,1H), 8.07 (s, 1H), 7.88 (s, | | | 0.1N HCl | | | |
| | | 1H), 7.60-7.50 (m, 4H), 7.12 (s, | | | | | | |
| | | 1H), 3.98 (s, 2H), 3.81 (d, J = 5.2 | | | | | | |
| | | Hz, 2H), 3.45 (s, 3H). | | | | | | |
| 410 | | ¹H-NMR (400 MHz, MeOD): δ9.55 | MeOD | 95 | Method G1 | 361.0 | t = 1.633 min | Method B |
| | | (s, 1H), 8.93(d, J = 5.8 Hz, 2H), | | | without | (M + 1) | | (NH4—HCO3) |
| | | (s, 1H), 8.36 (d, J = 4.4 Hz, 1H),8.04 | | | 0.1N HCl | | | |
| | | (d, J = 4.2 Hz, 1H), 7.86 (dd, J = | | | | | | |
| | | 6.6, 1.2 Hz, 1H), 7.69-7.57 (m, 3H), | | | | | | |
| | | 7.22 (d, J = 6.4 Hz, 1H), 4.11 (t, J = | | | | | | |
| | | 5.6 Hz, 2H), 3.98 (t, J = 5.6 Hz, 2H). | | | | | | |

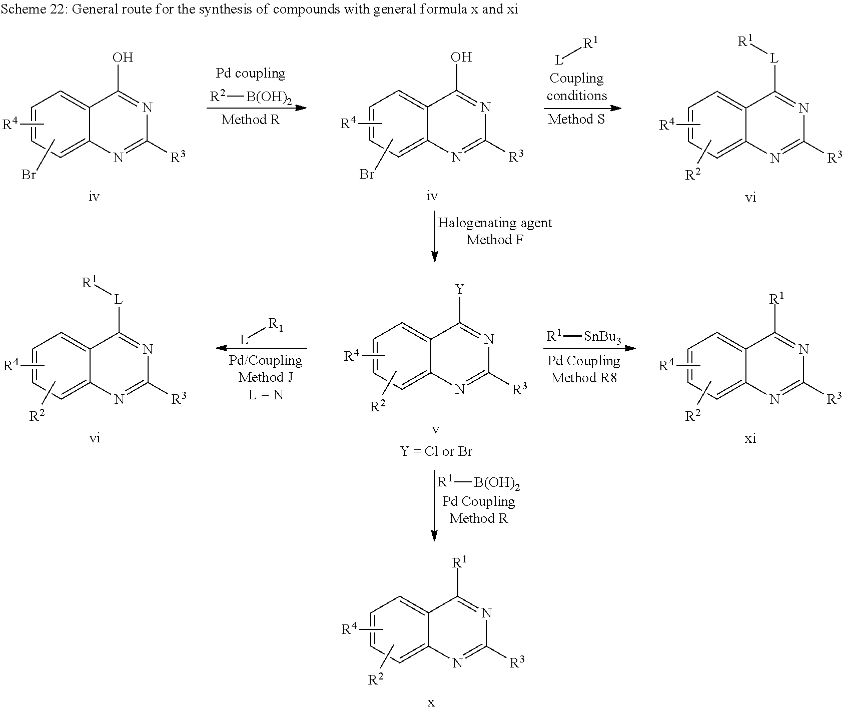

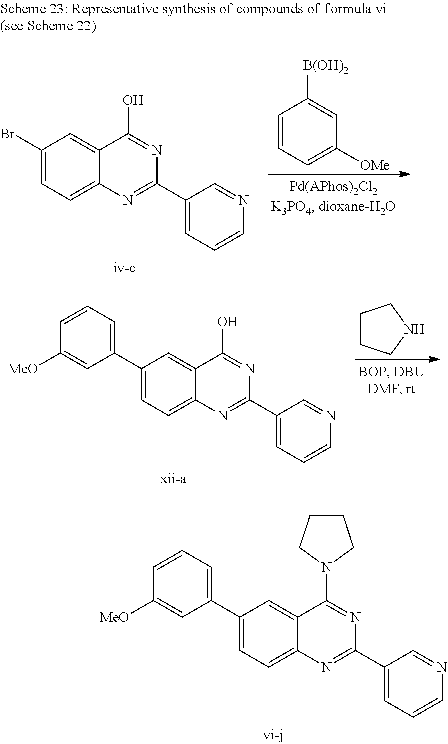 [SMILES: Oc1nc(-c2cccnc2)nc2ccc(Br)cc12 ; COc1cccc(B(O)O)c1 ; COc1cccc(-c2ccc3nc(-c4cccnc4)nc(O)c3c2)c1 ; C1CCNC1 ; COc1cccc(-c2ccc3nc(-c4cccnc4)nc(N4CCCC4)c3c2)c1]

### 6-(3-methoxyphenyl)-2-(pyridine-3-yl)quinazoline-4-ol (xii-a)

6-(3-methoxyphenyl)-2-(pyridine-3-yl)quinazoline-4-ol was prepared from 6-bromo-2-(pyridin-3-yl)quinazolin-4-ol (synthesis previously described in Scheme 7 method D) and coupling with 3-methoxylphenylboronic acid as described in Scheme 18 using Method R2. The resultant product, 6-(3-methoxyphenyl)-2-(pyridine-3-yl)quinazoline-4-ol, was a pale yellow solid (19.1 mg, 51%). LCMS m/z=344 (M+1) (Method C) (retention time=2.01 min). ¹H NMR (300 MHz, DMSO) δ 9.64 (d, J=1.3 Hz, 1H), 8.84-8.74 (m, 1H), 8.68 (dd, J=6.2, 1.7 Hz, 2H), 8.57 (d, J=1.6 Hz, 2H), 8.16 (ddd, J=14.4, 8.7, 2.2 Hz, 2H), 7.85 (d, J=8.7 Hz, 1H), 7.54 (dd, J=7.9, 4.8 Hz, 1H), 7.00 (d, J=8.7 Hz, 1H), 3.93 (s, 3H), 3.18 (d, J=4.3 Hz, 3H).

### 6-(3-methoxyphenyl)-2-(pyridine-3-yl)-4-(pyrrolidin-1-yl)quinazoline (vi-j)

6-(3-methoxyphenyl)-2-(pyridine-3-yl)-4-(pyrrolidin-1-yl)quinazoline was prepared from 6-(3-methoxyphenyl)-2-(pyridine-3-yl)quinazoline-4-ol and pyrrolidine in a manner analogous to that described for 6-bromo-8-methoxy-N-methyl-2-(pyridin-3-yl)quinazolin-4-amine using Method S in Scheme 21. 6-(3-methoxyphenyl)-2-(pyridine-3-yl)-4-(pyrrolidin-1-yl)quinazoline was obtained as a pale yellow solid (43 mg, 31%). LCMS m/z=383 (M+1) (Method C) (retention time=2.49 min) ¹H NMR (300 MHz, DMSO) δ 9.62 (s, 1H), 8.94 (d, J=5.0 Hz, 2H), 8.56 (s, 1H), 8.32 (dd, J=19.9, 8.5 Hz, 2H), 7.83 (s, 1H), 7.56-7.30 (m, 3H), 7.04 (d, J=6.8 Hz, 1H), 4.27 (s, 4H), 3.86 (s, 3H), 2.08 (s, 4H).

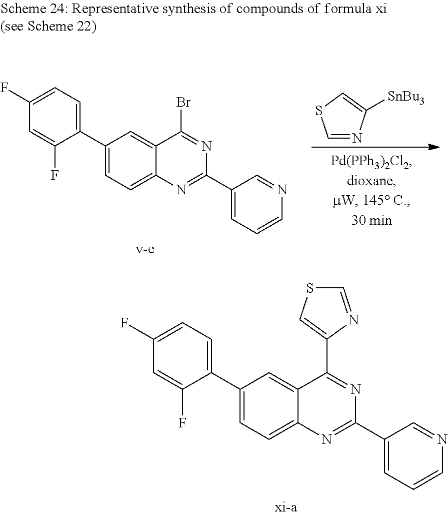

### Method R8: 4-(6-(2,4-difluorophenyl)-2-(pyridin-3-yl)quinazolin-4-yl)thiazole (xi-a)

To a 10 mL microwave vial were added 4-bromo-6-(2,4-difluorophenyl)-2-(pyridin-3-yl)quinazoline (0.200 g, 0.502 mmol), 4-(tributylstannyl)thiazole (0.282 g, 0.753 mmol) and trans-dichlorobis(triphenylphosphine)palladium (II) (Pd(PPh₃)₂Cl₂) (0.018 g, 0.025 mmol) in dioxane (2 ml) to give an orange suspension. The reaction mixture was heated to 145° C. for 30 min by microwave irradiation. LC-MS analysis of the crude mixture showed the reaction was completed. The reaction mixture was washed with water to yield a tan precipitate. The residue was purified via ISCO (silica gel, 97:3 CH₂Cl₂/MeOH, 24 gm column). The fractions collected were concentrated and dried under vacuum to give the title compound as an off-white powder (145.1 mg, 0.36 mmol, 72%). LC-MS m/z=403.1 (M+1) (retention time=2.60) ¹H NMR (300 MHz, DMSO) δ 9.81 (d, J=2.1 Hz, 1H), 9.64 (s, 1H), 9.47 (d, J=2.1 Hz, 1H), 9.21 (d, J=2.1 Hz, 1H), 8.95 (dd, J=9.9, 1.9 Hz, 1H), 8.77 (dd, J=4.4, 1.3 Hz, 1H), 8.20 (s, 2H), 7.75 (dd, J=15.5, 8.8 Hz, 1H), 7.68-7.58 (m, 2H), 7.58-7.42 (m, 2H), 7.29 (td, J=8.4, 2.5 Hz, 1H).

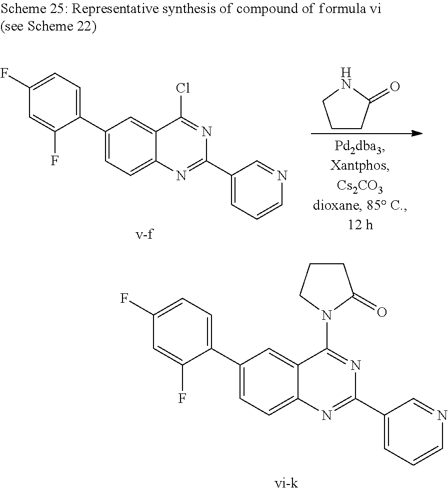 [SMILES: Fc1ccc(-c2ccc3nc(-c4cccnc4)nc(Cl)c3c2)c(F)c1 ; O=C1CCCN1 ; O=C1CCCN1c1nc(-c2cccnc2)nc2ccc(-c3ccc(F)cc3F)cc12]

### Method H1: 1-(6-(2,4-difluorophenyl)-2-(pyridin-3-yl)quinazolin-4-yl)pyrrolidin-2-one (vi-k)

To a 75 mL sealed tube were added 4-chloro-6-(2,4-difluorophenyl)-2-(pyridin-3-yl)quinazoline (0.5 g, 1.413 mmol), 2-pyrrolidone (0.130 ml, 1.696 mmol), tris(dibenzylideneacetone)dipalladium (0) (0.026 g, 0.028 mmol), xantphos (0.049 g, 0.085 mmol), and cesium carbonate (0.921 g, 2.83 mmol) in dioxane (15 ml) to give a green suspension. The reaction was heated at 85° C. overnight. LC-MS analysis of the crude mixture showed about 60% of product formed and 25% of the hydrolyzed lactam of parent compound formed. The reaction mixture was washed with water (80 mL), and the resulting green precipitate was filtered. The residue was purified via ISCO (silica gel, 97:3 methylene chloride/methanol, 40 gm column). The fractions collected were concentrated and dried under vacuum to yield the desired product as a white powder (169.6 mg, 0.42 mmol, 30%). LC-MS m/z=403.0 (M+1) (retention time=2.23) ¹H NMR (300 MHz, DMSO) δ 9.65 (d, J=1.2 Hz, 1H), 8.80 (dd, J=8.0, 1.7 Hz, 1H), 8.74 (dd, J=4.7, 1.6 Hz, 1H), 8.21-8.12 (m, 3H), 7.74-7.56 (m, 2H), 7.46 (ddd, J=11.7, 9.4, 2.5 Hz, 1H), 7.30 (td, J=8.5, 2.6 Hz, 1H), 4.28 (t, J=6.7 Hz, 2H), 2.69 (t, J=7.8 Hz, 2H), 2.33-2.15 (m, 2H).

The compounds in the following table were prepared in a manner analogous to that described in Scheme 22, replacing with the appropriate amine, stannane or lactam and 3-methoxyphenylboronic acid with the appropriate boronic acid.

TABLE 6

| | | | | | | | Puri- | Method | | Re- | |
|---|---|---|---|---|---|---|---|---|---|---|---|
| | | | | | | | ty | of | | ten- | LCMS |
| | Starting | Starting | | Salt | | ¹H NMR | per- | Cou- | | tion | Meth- |
| Number | Material 1 | Material 2 | Product | type | ¹H NMR | Solvent | cent | pling | LCMS | Time | od |
| 411 | 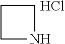 | 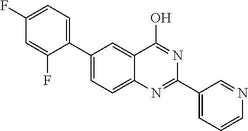 [SMILES: Oc1nc(-c2cccnc2)nc2ccc(-c3ccc(F)cc3F)cc12] | 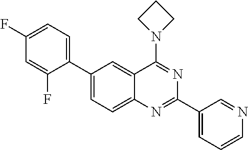 [SMILES: Fc1ccc(-c2ccc3nc(-c4cccnc4)nc(N4CCC4)c3c2)c(F)c1] | 2HCl | 1H NMR (300 MHz, DMSO) δ 9.58 (d, J = 1.9 Hz, 1H), 8.98 (d, J = 7.3 Hz, 1H), 8.93 (d, J = 5.0 Hz, 1H), 8.28 (d, J = 8.2 Hz, 1H), 8.22-8.09 (m, 2H), 7.90-7.74 (m, 2H), 7.47 (ddd, J = 10.6, | DMSO | 99 | Method S | 376 (M + 1 ) | 2.35 | C |
| | | | | | 8.7, 1.9 Hz, 1H), | | | | | | |
| | | | | | 7.28 (td, J = 8.5, 2.4 | | | | | | |
| | | | | | Hz, 1H), 4.86 (d, | | | | | | |
| | | | | | J = 105.9 Hz, 4H), | | | | | | |
| | | | | | 2.62-2.51 (m, 3H). | | | | | | |
| 412 | 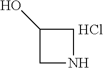 | 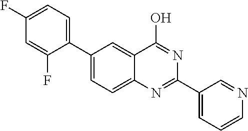 [SMILES: Oc1nc(-c2cccnc2)nc2ccc(-c3ccc(F)cc3F)cc12] | 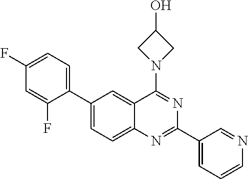 [SMILES: OC1CN(c2nc(-c3cccnc3)nc3ccc(-c4ccc(F)cc4F)cc23)C1] | 2HCl | 1H NMR (300 MHz, DMSO) δ 9.58 (d, J = 1.4 Hz, 1H), 8.99 (d, J = 7.4 Hz, 1H), 8.93 (d, J = 5.1 Hz, 1H), 8.26- 8.10 (m, 3H), 7.92- 7.75 (m, 2H), 7.47 (ddd, J = 11.6, 9.5, 2.4 Hz, 1H), 7.28 (td, J = 7.4, 1.4 Hz, 1H), 5.34-4.24 (m, | DMSO | 99 | Method S | 391.1 (M + 1) | 1.99 | C |
| | | | | | 6H). | | | | | | |
| 413 | 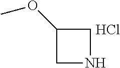 | 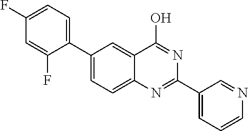 [SMILES: Oc1nc(-c2cccnc2)nc2ccc(-c3ccc(F)cc3F)cc12] | 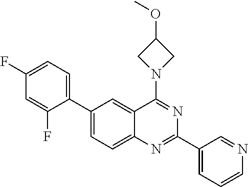 [SMILES: COC1CN(c2nc(-c3cccnc3)nc3ccc(-c4ccc(F)cc4F)cc23)C1] | 2HCl | 1H NMR (300 MHz, DMSO) δ 9.59 (d, J = 1.7 Hz, 1H), 9.02 (d, J = 8.0 Hz, 1H), 8.93 (d, J = 5.0 Hz, 1H), 8.25- 8.10 (m, 3H), 7.94- 7.76 (m, 2H), 7.47 (ddd, J = 10.4, 8.9, 1.9 Hz, 1H), 7.28 (td, J = 8.5, 2.6 Hz, 1H), 5.31-4.35 (m, 5H), | DMSO | 99 | Method S | 406.1 (M + 1) | 2.27 | C |
| | | | | | 3.32 (s, J = 20.1 Hz, | | | | | | |
| | | | | | 3H). | | | | | | |
| 414 | 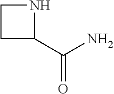 | 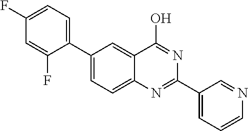 [SMILES: Oc1nc(-c2cccnc2)nc2ccc(-c3ccc(F)cc3F)cc12] | 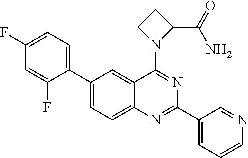 [SMILES: NC(=O)C1CCN1c1nc(-c2cccnc2)nc2ccc(-c3ccc(F)cc3F)cc12] | 2HCl | 1H NMR (300 MHz, DMSO) δ 9.58 (S, 1H), 9.05 (d, J = 7.5 Hz, 1H), 8.94 (d, J = 5.0 Hz, 1H), 8.28- 8.04 (m, 3H), 7.98- 7.85 (m, 2H), 7.80 (s, 1H), 7.53-7.35 (m, 2H), 7.27 (td, J = 7.1, 1.3 Hz, 1H), | DMSO | 99 | Method S | 418.1 (M + 1 ) | 1.90 | C |
| | | | | | 5.43-4.52 (m, | | | | | | |
| | | | | | 3H), 2.79 (s, 1H). | | | | | | |
| 415 | 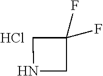 | 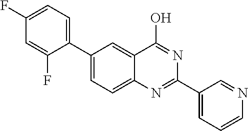 [SMILES: Oc1nc(-c2cccnc2)nc2ccc(-c3ccc(F)cc3F)cc12] | 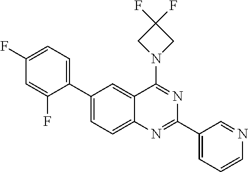 [SMILES: Fc1ccc(-c2ccc3nc(-c4cccnc4)nc(N4CC(F)(F)C4)c3c2)c(F)c1] | 2HCl | 1H NMR (300 MHz, DMSO) δ 9.67 (d, J = 1.3 Hz, 1H), 9.22 (d, J = 8.2 Hz, 1H), 8.97 (d, J = 5.3 Hz, 1H), 8.19- 7.95 (m, 4H), 7.83 (dd, J = 15.7, 8.8 Hz, 1H), 7.52-7.41 (m, 1H), 7.27 (td, J = 8.7, 2.8 Hz, 1H), 5.22 (t, | DMSO | 99 | Method S | 412.1 (M + 1) | 2.38 | C |
| | | | | | J = 12.0 Hz, 4H). | | | | | | |
| 416 | 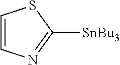 | 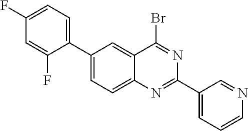 [SMILES: Fc1ccc(-c2ccc3nc(-c4cccnc4)nc(Br)c3c2)c(F)c1] | 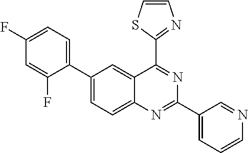 [SMILES: Fc1ccc(-c2ccc3nc(-c4cccnc4)nc(-c4nccs4)c3c2)c(F)c1] | 2HCl | 1H NMR (300 MHz, DMSO) δ 9.89 (s, 1H), 9.75 (s, 1H), 9.09 (d, J = 8.0 Hz, 1H), 8.91 (d, J = 4.0 Hz, 1H), 8.33 (d, J = 3.2 Hz, 1H), 8.32- 8.20 (m, 3H), 7.89 (dd, J = 7.9, 5.7 Hz, 1H), 7.78 (dd, J = | DMSO | 99 | Method R8 | 404.1 (M + 1) | 2.77 | C |
| | | | | | 15.6, 8.9 Hz, 1H), | | | | | | |
| | | | | | 7.66-7.45 (m, 2H), | | | | | | |
| | | | | | 7.32 (td, J = | | | | | | |
| | | | | | 8.4, 2.4 Hz, 1H). | | | | | | |
| 417 | 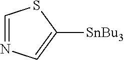 | 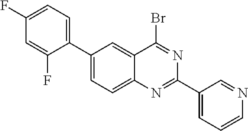 [SMILES: Fc1ccc(-c2ccc3nc(-c4cccnc4)nc(Br)c3c2)c(F)c1] | 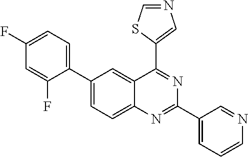 [SMILES: Fc1ccc(-c2ccc3nc(-c4cccnc4)nc(-c4cncs4)c3c2)c(F)c1] | 2HCl | 1H NMR (300 MHz, DMSO) δ 9.73 (s, 1H), 9.50 (s, 1H), 9.16 (d, J = 7.6 Hz, 1H), 9.01 (s, 1H), 8.93 (d, J = 5.0 Hz, 1H), 8.64 (s, 1H), 8.38-8.23 (m, 2H), 8.01-7.86 (m, 2H), 7.67-7.43 (m, 2H), | DMSO | 99 | Method R8 | 404.1 (M + 1) | 2.50 | C |
| | | | | | 7.30 (td, J = 8.8, 2.5 | | | | | | |
| | | | | | Hz, 1H). | | | | | | |
| 418 | 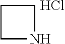 | 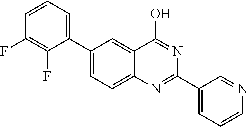 [SMILES: Oc1nc(-c2cccnc2)nc2ccc(-c3cccc(F)c3F)cc12] | 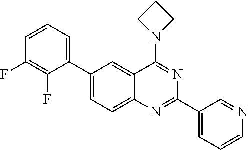 [SMILES: Fc1cccc(-c2ccc3nc(-c4cccnc4)nc(N4CCC4)c3c2)c1F] | 2HCl | 1H NMR (300 MHz, DMSO) δ 9.58 (d, J = 1.3 Hz, 1H), 9.02-8.90 (m, 2H), 8.30-8.14 (m, 3H), 7.85 (dd, J = 7.1, 5.5 Hz, 1H), 7.61-7.48 (m, 2H), 7.38 (dd, J = 14.1, 6.8 Hz, 1H), 5.47- 4.38 (m, 4H), 2.55 (s, J = 10.8 Hz, 2H). | DMSO | 99 | Method S | 376.1 (M + 1) | 2.53 | C |
| 419 | 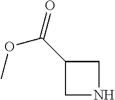 | 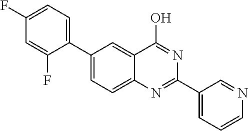 [SMILES: Oc1nc(-c2cccnc2)nc2ccc(-c3ccc(F)cc3F)cc12] | 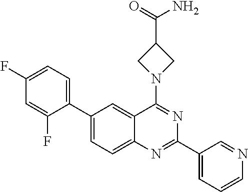 [SMILES: NC(=O)C1CN(c2nc(-c3cccnc3)nc3ccc(-c4ccc(F)cc4F)cc23)C1] | | 1H NMR (300 MHz, DMSO) δ 9.58 (d, J = 1.3 Hz, 1H), 8.73 (dd, J = 8.0, 1.7 Hz, 1H), 8.68 (dd, J = 4.8, 1.7 Hz, 1H), 8.05 (s, 1H), 7.97 (d, J = 8.9 Hz, 1H), 7.89 (d, J = 8.6 Hz, 1H), 7.78 (td, J = 8.9, 6.7 Hz, 1H), 7.61 (s, 1H), 7.57- | DMSO | 99 | Method S followed by amidation of the methyl ester using NH₄OH in THF | 418.1 (M + 1) | 1.79 | C |
| | | | | | 7.50 (m, 1H), 7.42 | | | | | | |
| | | | | | (ddd, J = 10.8, 8.8, | | | | | | |
| | | | | | 1.8 Hz, 1H), 7.24 | | | | | | |
| | | | | | (td, J = 9.4, 3.0 Hz, | | | | | | |
| | | | | | 1H), 7.17 (s, 1H), | | | | | | |
| | | | | | 4.70 (d, J = 44.1 Hz, | | | | | | |
| | | | | | 4H), 3.63-3.50 (m, | | | | | | |
| | | | | | 1H). | | | | | | |
| 420 | 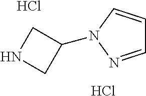 | 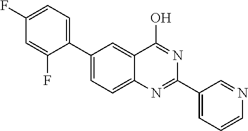 [SMILES: Oc1nc(-c2cccnc2)nc2ccc(-c3ccc(F)cc3F)cc12] | 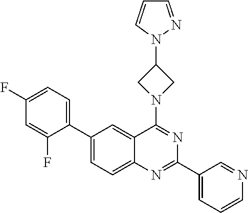 [SMILES: Fc1ccc(-c2ccc3nc(-c4cccnc4)nc(N4CC(n5cccn5)C4)c3c2)c(F)c1] | | 1H NMR (300 MHz, DMSO) δ 9.60 (d, J = 1.2 Hz, 1H), 8.75 (dd, J = 7.9, 1.7 Hz, 1H), 8.68 (dd, J = 4.7, 1.7 Hz, 1H), 8.08 (s, 1H), 8.04-7.90 (m, 3H), 7.79 (dt, J = 9.0, 6.7 Hz, 1H), 7.58 (d, J = 1.6 Hz, 1H), 7.53 (dd, J = 7.9, 4.8 Hz, 1H), 7.40 (ddd, J = | DMSO | 99 | Method S | 441.1 (M + 1) | 2.26 | C |
| | | | | | 11.7, 9.4, 2.4 Hz, | | | | | | |
| | | | | | 1H), 7.21 (td, J = | | | | | | |
| | | | | | 8.5, 2.5 Hz, 1H), | | | | | | |
| | | | | | 6.33 (s, J = 1.8 Hz, | | | | | | |
| | | | | | 1H), 5.54 (s, 1H), | | | | | | |
| | | | | | 5.14 (s, 2H), 4.91 | | | | | | |
| | | | | | (s, 2H). | | | | | | |
| 421 | 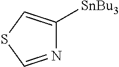 | 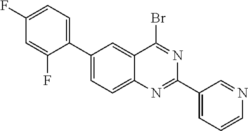 [SMILES: Fc1ccc(-c2ccc3nc(-c4cccnc4)nc(Br)c3c2)c(F)c1] | 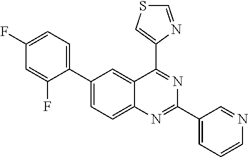 [SMILES: Fc1ccc(-c2ccc3nc(-c4cccnc4)nc(-c4cscn4)c3c2)c(F)c1] | | 1H NMR (300 MHz, DMSO) δ 9.81 (d, J = 2.1 Hz, 1H), 9.64 (s, 1H), 9.47 (d, J = 2.1 Hz, 1H), 9.21 (d, J = 2.1 Hz, 1H), 8.95 (dd, J = 9.9, 1.9 Hz, 1H), 8.77 (dd, J = 4.4, 1.3 Hz, 1H), 8.20 (s, 2H), 7.75 | DMSO | 99 | Method R8 | 403.3 (M + 1) | 2.55 | C |
| | | | | | (dd, J = 15.5, 8.8 Hz, | | | | | | |
| | | | | | 1H), 7.68-7.58 (m, | | | | | | |
| | | | | | 2H), 7.58-7.42 (m, | | | | | | |
| | | | | | 2H), 7.29 (td, J = | | | | | | |
| | | | | | 8.4, 2.5 Hz, 1H). | | | | | | |
| 422 | 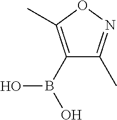 | 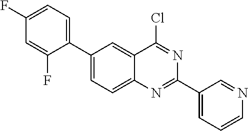 [SMILES: Fc1ccc(-c2ccc3nc(-c4cccnc4)nc(Cl)c3c2)c(F)c1] | 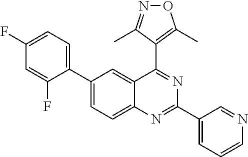 [SMILES: Cc1noc(C)c1-c1nc(-c2cccnc2)nc2ccc(-c3ccc(F)cc3F)cc12] | | 1H NMR (300 MHz, DMSO) δ 9.69 (d, J = 0.7 Hz, 1H), 8.84 (d, J = 7.9 Hz, 1H), 8.76 (d, J = 4.7 Hz, 1H), 8.33-8.20 (m, 2H), 8.02 (s, 1H), 7.73 (dd, J = 15.5, 8.9 Hz, 1H), 7.62 (dd, J = 7.9, 4.8 Hz, | DMSO | 99 | Method R8 | 415.3 (M + 1) | 2.45 | C |
| | | | | | 1H), 7.45 (ddd, | | | | | | |
| | | | | | J = 11.4, 9.4, 2.0 | | | | | | |
| | | | | | Hz, 1H), 7.25 (td, | | | | | | |
| | | | | | J = 8.6, 2.2 Hz, 1H), | | | | | | |
| | | | | | 2.45 (s, 3H), 2.33 | | | | | | |
| | | | | | (s, 3H). | | | | | | |
| 423 | 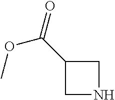 | 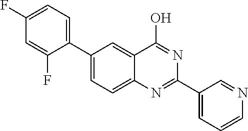 [SMILES: Oc1nc(-c2cccnc2)nc2ccc(-c3ccc(F)cc3F)cc12] | 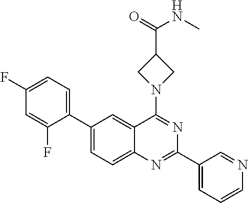 [SMILES: CNC(=O)C1CN(c2nc(-c3cccnc3)nc3ccc(-c4ccc(F)cc4F)cc23)C1] | | 1H NMR (300 MHz, DMSO) δ 9.58 (d, J = 1.3 Hz, 1H), 8.72 (dd, J = 6.2, 1.9 Hz, 1H), 8.67 (dd, J = 5.0, 1.5 Hz, 1H), 8.08 (d, J = 4.7 Hz, 1H), 8.04 (s, 1H), 7.97 (d, J = 8.6 Hz, 1H), 7.89 (d, J = 8.7 Hz, 1H), 7.78 (td, J = 8.7, 7.0 Hz, 1H), 7.53 (dd, J = 7.9, 4.8 | DMSO | 99 | Method S followed by amidation of the methyl ester using MeNH2 in THF | No LCMS Data | | |
| | | | | | Hz, 1H), 7.42 (ddd, | | | | | | |
| | | | | | J = 9.8, 6.4, 1.8 Hz, | | | | | | |
| | | | | | 1H), 7.23 (td, J = | | | | | | |
| | | | | | 8.4, 2.4 Hz, 1H), | | | | | | |
| | | | | | 4.70 (d, J = 38.4 Hz, | | | | | | |
| | | | | | 4H), 3.64-3.48 (m, | | | | | | |
| | | | | | 1H), 2.63 (d, J = 4.6 | | | | | | |
| | | | | | Hz, 3H). | | | | | | |
| 424 | 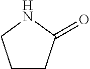 | 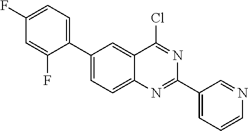 [SMILES: Fc1ccc(-c2ccc3nc(-c4cccnc4)nc(Cl)c3c2)c(F)c1] | 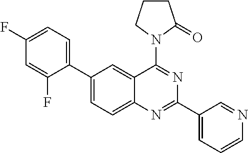 [SMILES: O=C1CCCN1c1nc(-c2cccnc2)nc2ccc(-c3ccc(F)cc3F)cc12] | | 1H NMR (300 MHz, DMSO) δ 9.65 (d, J = 1.2 Hz, 1H), 8.80 (dd, J = 8.0, 1.7 Hz, 1H), 8.74 (dd, J = 4.7, 1.6 Hz, 1H), 8.21-8.12 (m, 3H), 7.74-7.56 (m, 2H), 7.46 (ddd, J = 11.7, 9.4, 2.5 Hz, 1H), | DMSO | 99 | Method H1 | 403.0 (M + 1) | 2.23 | C |
| | | | | | 7.30 (td, J = 8.5, 2.6 | | | | | | |
| | | | | | Hz, 1H), 4.28 (t, J = | | | | | | |
| | | | | | 6.7 Hz, 2H), 2.69 (t, | | | | | | |
| | | | | | J = 7.8 Hz, 2H), | | | | | | |
| | | | | | 2.33-2.15 (m, 2H). | | | | | | |
| 425 | 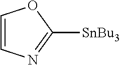 | 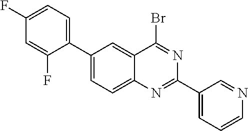 [SMILES: Fc1ccc(-c2ccc3nc(-c4cccnc4)nc(Br)c3c2)c(F)c1] | 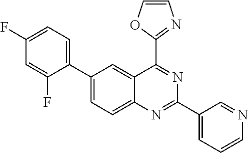 [SMILES: Fc1ccc(-c2ccc3nc(-c4cccnc4)nc(-c4ncco4)c3c2)c(F)c1] | | 1H NMR (300 MHz, DMSO) δ 9.75 (d, J = 1.4 Hz, 1H), 9.69 (s, 1H), 9.12 (d, J = 8.1 Hz, 1H), 8.90 (dd, J = 5.4, 1.2 Hz, 1H), 8.64 (s, 1H), 8.29 (s, 2H), 7.89 (dd, J = 7.8, 5.3 Hz, 1H), | DMSO | 99 | Method R8 | 387.1 (M + 1) | 2.39 | C |
| | | | | | 7.85-7.73 (m, 2H), | | | | | | |
| | | | | | 7.66-7.45 (m, 3H). | | | | | | |
| 426 | 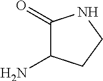 | 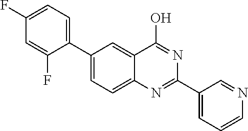 [SMILES: Oc1nc(-c2cccnc2)nc2ccc(-c3ccc(F)cc3F)cc12] | 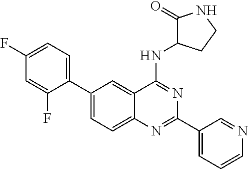 [SMILES: O=C1NCCC1Nc1nc(-c2cccnc2)nc2ccc(-c3ccc(F)cc3F)cc12] | | 1H NMR (300 MHz, DMSO) δ 9.59 (d, J = 2.0 Hz, 1H), 8.82-8.71 (m, 2H), 8.67 (dd, J = 4.7, 1.7 Hz, 1H), 8.50 (s, 1H), 8.04 (s, 1H), 7.96 (d, J = 8.6 Hz, 1H), 7.87 (d, J = 8.7 Hz, IH), 7.72 (dt, J = 8.9, | DMSO | 99 | Method S | 418.1 (M + 1) | 1.86 | C |
| | | | | | 6.7 Hz, 1H), 7.57- | | | | | | |
| | | | | | 7.40 (m, 2H), 7.28 | | | | | | |
| | | | | | (td, J = 8.5, 2.6 | | | | | | |
| | | | | | Hz, 1H), 5.11 (dd, | | | | | | |
| | | | | | J = 17.7, 9.1 Hz, | | | | | | |
| | | | | | 1H), 3.37 (s, 2H), | | | | | | |
| | | | | | 2.34-2.15 (m, 1H). | | | | | | |
| 427 | 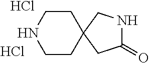 [SMILES: Cl ; Cl ; O=C1CC2(CCNCC2)CN1] | 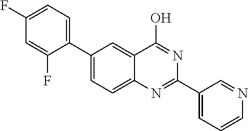 [SMILES: Oc1nc(-c2cccnc2)nc2ccc(-c3ccc(F)cc3F)cc12] | 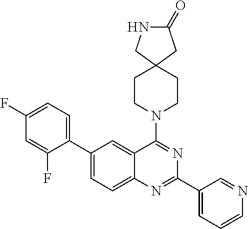 [SMILES: O=C1CC2(CCN(c3nc(-c4cccnc4)nc4ccc(-c5ccc(F)cc5F)cc34)CC2)CN1] | | 1H NMR (300 MHz, DMSO) δ 9.60 (d, J = 1.2 Hz, 1H), 8.74 (dd, J = 7.5, 1.6 Hz, 1H), 8.69 (dd, J = 4.5, 1.4 Hz, 1H), 8.07 (s, 1H), 7.97 (s, 2H), 7.76 (dt, J = 9.0, 6.7 Hz, 1H), 7.59 (s, 1H), 7.55 (dd, J = 7.9, 4.8 Hz, 1H), 7.45 (ddd, J = 11.7, 9.3, 2.4 Hz, 1H), 7.26 (td, J = | DMSO | 99 | Method S | 472.2 (M + 1) | 1.96 | C |
| | | | | | 8.9, 2.5 Hz, 1H), | | | | | | |
| | | | | | 3.97 (d, J = 13.5 Hz, | | | | | | |
| | | | | | 2H), 3.81 (d, J = | | | | | | |
| | | | | | 13.9 Hz, 2H), 3.15 | | | | | | |
| | | | | | (s, J = 2.0 Hz, | | | | | | |
| | | | | | 2H), 2.18 (s, 2H), | | | | | | |
| | | | | | 1.79 (s, 4H). | | | | | | |
| 428 | 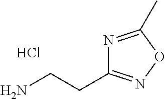 | 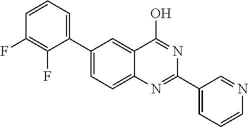 [SMILES: Oc1nc(-c2cccnc2)nc2ccc(-c3cccc(F)c3F)cc12] | 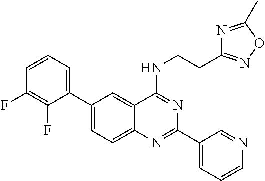 [SMILES: Cc1nc(CCNc2nc(-c3cccnc3)nc3ccc(-c4cccc(F)c4F)cc23)no1] | | | DMSO | 98 | Method S | | | C |
| 429 | 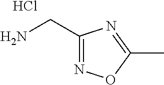 | 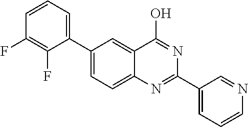 [SMILES: Oc1nc(-c2cccnc2)nc2ccc(-c3cccc(F)c3F)cc12] | 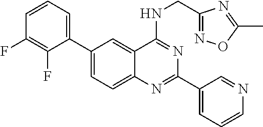 [SMILES: Cc1nc(CNc2nc(-c3cccnc3)nc3ccc(-c4cccc(F)c4F)cc23)no1] | | | DMSO | 98 | Method S | | | C |
| 430 | 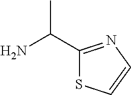 | 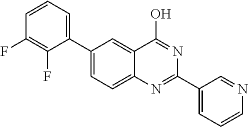 [SMILES: Oc1nc(-c2cccnc2)nc2ccc(-c3cccc(F)c3F)cc12] | 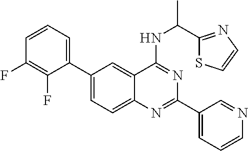 [SMILES: CC(Nc1nc(-c2cccnc2)nc2ccc(-c3cccc(F)c3F)cc12)c1nccs1] | | LCMS only | DMSO | 98 | Method S | | | C |
| 431 | 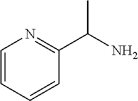 | 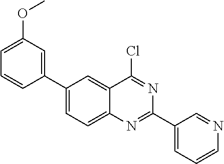 [SMILES: COc1cccc(-c2ccc3nc(-c4cccnc4)nc(Cl)c3c2)c1] | 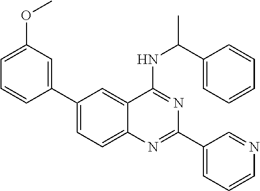 [SMILES: COc1cccc(-c2ccc3nc(-c4cccnc4)nc(NC(C)c4ccccc4)c3c2)c1] | 3 HCl | ¹H NMR (400 MHz, DMSO) δ 10.29 (s, 1H), 10.20 (br s, 1H), 9.58 (s, 1H), 9.30-9.16 (m, 2H), 8.97 (d, J = 5.3 Hz, 1H), 8.83 (d, J = 5.3 Hz, 1H), 8.43 (d, J = 7.3 Hz, 1H), 8.39- 8.34 (m, 1H), 8.28 (d, J = 6.9 Hz, | DMSO | >98 | Method L, F3, G1 without HCl | | | |
| | | | | | 1H), 8.15 (d, J = | | | | | | |
| | | | | | 8.6 Hz, 1H), 8.04- | | | | | | |
| | | | | | 7.96 (m, 1H), 7.83 | | | | | | |
| | | | | | (d, J = 5.6 Hz, 1H), | | | | | | |
| | | | | | 7.62-7.55 (m, 2H), | | | | | | |
| | | | | | 7.50 (t, J = 7.8 Hz, | | | | | | |
| | | | | | 1H), 7.11-6.98 (m, | | | | | | |
| | | | | | 1H), 6.26-6.11 (m, | | | | | | |
| | | | | | 1H), 1.90 (d, J = | | | | | | |
| | | | | | 7.1 Hz, 3H). | | | | | | |
| 432 | 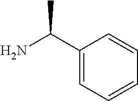 | 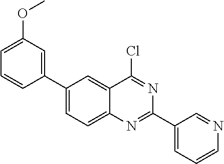 [SMILES: COc1cccc(-c2ccc3nc(-c4cccnc4)nc(Cl)c3c2)c1] | 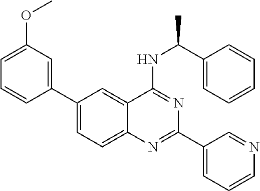 [SMILES: COc1cccc(-c2ccc3nc(-c4cccnc4)nc(N[C@@H](C)c4ccccc4)c3c2)c1] | HCl | ¹H NMR (400 MHz, DMSO) δ 10.30 (s, 1H), 9.57 (d, J = 1.7 Hz, 1H), 9.11 (s, 1H), 9.04 (d, J = 8.1 Hz, 1H), 8.95 (dd, J = 5.2, 1.4 Hz, 1H), 8.37 (dd, J = 8.7, 1.6 Hz, 1H), 8.21 (d, J = 8.6 Hz, 1H), 7.91 (dd, J = | DMSO | >98 | Method L, F3, G1 without HCl | | | |
| | | | | | 8.1, 5.2 Hz, 1H), | | | | | | |
| | | | | | 7.66-7.59 (m, | | | | | | |
| | | | | | 2H), 7.55-7.45 | | | | | | |
| | | | | | (m, 3H), 7.42- | | | | | | |
| | | | | | 7.33 (m, 2H), 7.30- | | | | | | |
| | | | | | 7.21 (m, 1H), | | | | | | |
| | | | | | 7.10-7.03 (m, 1H), | | | | | | |
| | | | | | 5.99-5.86 (m, 1H), | | | | | | |
| | | | | | 3.90 (s, 3H), 1.76 (d, | | | | | | |
| | | | | | J = 7.0 Hz ,3H). | | | | | | |
| 433 | 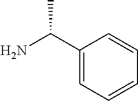 | 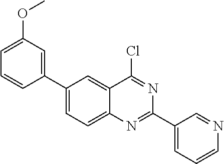 [SMILES: COc1cccc(-c2ccc3nc(-c4cccnc4)nc(Cl)c3c2)c1] | 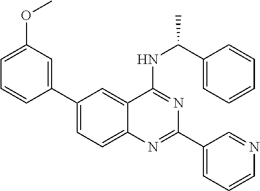 [SMILES: COc1cccc(-c2ccc3nc(-c4cccnc4)nc(N[C@H](C)c4ccccc4)c3c2)c1] | HCl | ¹H NMR (400 MHz, DMSO) δ 10.09 (s, 1H), 9.54 (d, J = 1.7 Hz, 1H), 9.05 (s, 1H), 9.02- 8.96 (m, 1H), 8.93 (dd, J = 5.1, 1.5 Hz, 1H), 8.36 (dd, J = 8.5, 1.2 Hz, 1H), 8.13 (d, J = 9.3 Hz, 1H), 7.93- 7.83 (m, 1H), | DMSO | >98 | Method L, F3, G1 without HCl | | | |
| | | | | | 7.65-7.57 (m, 2H), | | | | | | |
| | | | | | 7.54-7.44 (m, 3H), | | | | | | |
| | | | | | 7.42-7.33 (m, 2H), | | | | | | |
| | | | | | 7.30-7.22 (m, 1H), | | | | | | |
| | | | | | 7.12-7.03 (m, 1H), | | | | | | |
| | | | | | 5.97-5.88 (m, 1H), | | | | | | |
| | | | | | 3.89 (s, 3H), 1.75 (d, | | | | | | |
| | | | | | J = 7.0 Hz, 3H). | | | | | | |
| 434 | 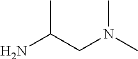 | 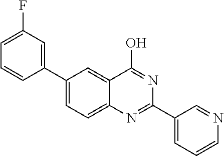 [SMILES: Oc1nc(-c2cccnc2)nc2ccc(-c3cccc(F)c3)cc12] | 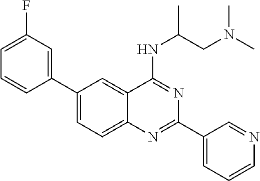 [SMILES: CC(CN(C)C)Nc1nc(-c2cccnc2)nc2ccc(-c3cccc(F)c3)cc12] | 3 HCl | 1H NMR (400 MHz, DMSO) δ 10.36 (brs, 2H), 9.74 (d, J = 1.6 Hz, 1H), 9.23 (brs, 2H), 8.98 (dd, J = 5.2, 1.5 Hz, 1H), 8.42 (dd, J = 8.8, 1.7 Hz, 1H), 8.28 (d, J = 8.5 Hz, 1H), 8.03- 7.86 (m, 3H), 7.65- 7.53 (m, 1H), | DMSO | >98 | Method F2, Coupling with DIPEA in dioxane at 100° C. | | | |
| | | | | | 7.35-7.24 (m, 1H), | | | | | | |
| | | | | | 5.45-5.34 (m, | | | | | | |
| | | | | | 1H), 3.95-3.76 | | | | | | |
| | | | | | (m, 1H), 3.49- | | | | | | |
| | | | | | 3.33 (m, 1H), 2.84 | | | | | | |
| | | | | | (dd, J = 15.3, 4.8 | | | | | | |
| | | | | | Hz, 6H), 1.45 (d, | | | | | | |
| | | | | | J = 6.6 Hz, 3H). | | | | | | |
| 435 | 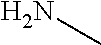 | 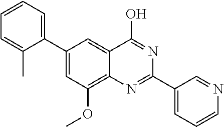 [SMILES: COc1cc(-c2ccccc2C)cc2c(O)nc(-c3cccnc3)nc12] | 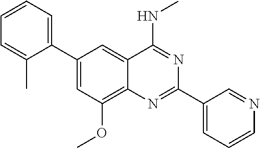 [SMILES: CNc1nc(-c2cccnc2)nc2c(OC)cc(-c3ccccc3C)cc12] | 2 HCl | 1H NMR (400 MHz, DMSO) δ 9.56 (d, J = 1.7 Hz, 1H), 9.26 (br-s, 1H), 9.18 (d, J = 8.0 Hz, 1H), 8.99 (dd, J = 5.4, 1.4 Hz, 1H), 8.06 (dd, J = 8.0, 5.5 Hz, 1H), 7.92 | DMSO | >98 | Method S | | | |
| | | | | | (d, J = 1.3 Hz, 1H), | | | | | | |
| | | | | | 7.46-7.27 (m, | | | | | | |
| | | | | | 5H), 4.05 (s, 3H), | | | | | | |
| | | | | | 3.20 (d, J = 4.3 | | | | | | |
| | | | | | Hz, 3H), 2.34 (s, 3H). | | | | | | |
| 436 | 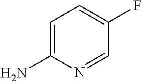 | 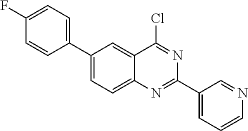 [SMILES: Fc1ccc(-c2ccc3nc(-c4cccnc4)nc(Cl)c3c2)cc1] | 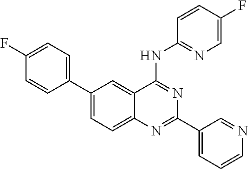 [SMILES: Fc1ccc(-c2ccc3nc(-c4cccnc4)nc(Nc4ccc(F)cn4)c3c2)cc1] | 2HCl | 1H NMR (400 MHz, DMSO) δ 11.20 (s, 1H), 9.60 (s, 1H), 9.17 (d, J = 8.2 Hz, 1H), 9.13 (d, J = 1.7 Hz, 1H), 8.97 (d, J = 4.2 Hz, 1H), 8.54 (d, J = 3.1Hz, 1H), 8.48 (dd, J = 9.2, 4.1 Hz, 1H), 8.35 (dd, | DMSO | >98 | Method R4 F3 H1 | | | |
| | | | | | J = 8.8, 1.8 Hz, | | | | | | |
| | | | | | 1H), 8.10-8.01 | | | | | | |
| | | | | | (m, 4H), 7.98- | | | | | | |
| | | | | | 7.91 (m, 1H), | | | | | | |
| | | | | | 7.45-7.38 (m, 2H). | | | | | | |
| 437 | 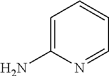 | 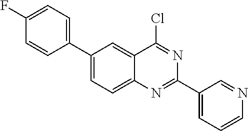 [SMILES: Fc1ccc(-c2ccc3nc(-c4cccnc4)nc(Cl)c3c2)cc1] | 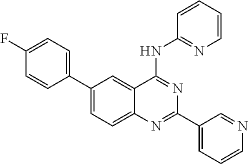 [SMILES: Fc1ccc(-c2ccc3nc(-c4cccnc4)nc(Nc4ccccn4)c3c2)cc1] | 2HCl | 1H NMR (400 MHz, DMSO) δ 11.53 (s, 1H), 9.62 (d, J = 1.7 HZ, 1H), 9.27-9.16 (m, 2H), 8.99 (dd, J = 5.4, 1.3 Hz, 1H), 8.58 (dd, J = 5.1, 1.1 Hz, 1H), 8.51 (d, J = 8.4 Hz, 1H), | DMSO | >98 | Method R4 F3 H1 | | | |
| | | | | | 8.37 (dd, J = 8.8, | | | | | | |
| | | | | | 1.9 Hz, 1H), 8.18- | | | | | | |
| | | | | | 8.01 (m, 5H), | | | | | | |
| | | | | | 7.45-7.35 (m, 3H). | | | | | | |
| 438 | 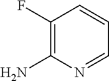 | 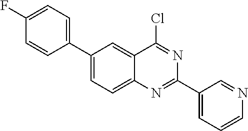 [SMILES: Fc1ccc(-c2ccc3nc(-c4cccnc4)nc(Cl)c3c2)cc1] | 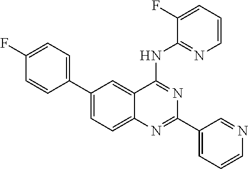 [SMILES: Fc1ccc(-c2ccc3nc(-c4cccnc4)nc(Nc4ncccc4F)c3c2)cc1] | 2HCl | 1H NMR (400 MHz, DMSO) δ 11.14 (s, 1H), 9.38 (d, J = 1.6 Hz, 1H), 9.04-8.99 (m, 1H), 8.95-8.84 (m, 2H), 8.46-8.42 (m, 1H), 8.36 (dd, J = 8.7, 1.9 Hz, 1H), 8.10-7.95 (m, 4H), 7.95-7.86 | DMSO | >98 | Method R4 F3 H1 | | | |
| | | | | | (m, 1H), 7.59- | | | | | | |
| | | | | | 7.52 (m, 1H), 7.47- | | | | | | |
| | | | | | 7.39 (m, 2H). | | | | | | |
| 439 | 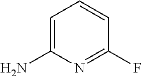 | 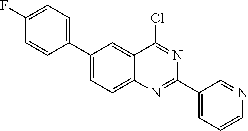 [SMILES: Fc1ccc(-c2ccc3nc(-c4cccnc4)nc(Cl)c3c2)cc1] | 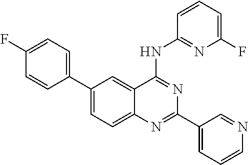 [SMILES: Fc1ccc(-c2ccc3nc(-c4cccnc4)nc(Nc4cccc(F)n4)c3c2)cc1] | HCl | 1H NMR (400 MHz, DMSO) δ 11.09 (s, 1H), 9.61 (d, J = 1.7 Hz, 1H), 9.14-9.07 (m, 2H), 8.93 (dd, J = 5.2, 1.4 Hz, 1H), 8.48 (dd, J = 8.0, 2.2 Hz, 1H), 8.32 (dd, J = 8.8, 1.9 Hz, 1H), 8.16 (dd, | DMSO | >98 | Method R4 F3 H1 | | | |
| | | | | | J = 16.7, 8.1 Hz, | | | | | | |
| | | | | | 1H), 8.09-8.01 (m, | | | | | | |
| | | | | | 3H), 7.96 (dd, J = | | | | | | |
| | | | | | 8.0, 5.3 Hz, 1H), | | | | | | |
| | | | | | 7.44-7.36 (m, | | | | | | |
| | | | | | 2H), 6.99 (dd, J = | | | | | | |
| | | | | | 7.9, 2.5 Hz, 1H). | | | | | | |
| 440 | 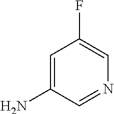 | 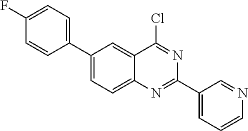 [SMILES: Fc1ccc(-c2ccc3nc(-c4cccnc4)nc(Cl)c3c2)cc1] | 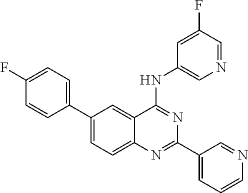 [SMILES: Fc1ccc(-c2ccc3nc(-c4cccnc4)nc(Nc4cncc(F)c4)c3c2)cc1] | HCl | 1H NMR (400 MHz, DMSC) δ 10.89 (s, 1H), 9.51 (d, J = 1.6 Hz, 1H), 9.13-9.08 (m, 1H), 9.08-9.03 (m, 2H), 8.95 (dd, J = 5.3, 1.3 Hz, 1H), 8.49-8.42 (m, 2H), 8.32 (dd, J = 8.7, 1.9 Hz, 1H), 8.08-8.00 (m, 4H), | DMSO | >98 | Method R4 F3 H1 | | | |
| | | | | | 7.46-7.39 (m, 2H). | | | | | | |
| 441 | 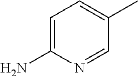 | 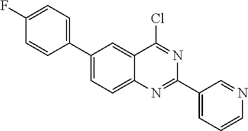 [SMILES: Fc1ccc(-c2ccc3nc(-c4cccnc4)nc(Cl)c3c2)cc1] | 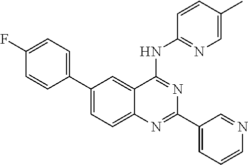 [SMILES: Cc1ccc(Nc2nc(-c3cccnc3)nc3ccc(-c4ccc(F)cc4)cc23)nc1] | 2HCl | 1H NMR (400 MHz, DMSO) δ 11.52 (s, 1H), 9.60 (s, 1H), 9.22-9.13 (m, 2H), 8.97 (d, J = 4.3 Hz, 1H), 8.42 (d, J = 2.2 Hz, 1H), 8.41-8.34 (m, 2H), 8.12-8.05 (m, 3H), 3.03 (dd, J = 8.0, 5.4 Hz, 1H), | DMSO | >98 | Method R4 F3 H1 | | | |
| | | | | | 7.99 (dd, J = 8.6, 1.9 | | | | | | |
| | | | | | Hz, 1H), 7.44-7.37 | | | | | | |
| | | | | | (m, 2H), 2.40 (s, 3H). | | | | | | |
| 442 | 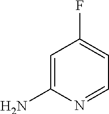 | 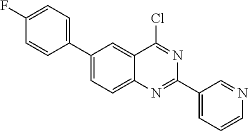 [SMILES: Fc1ccc(-c2ccc3nc(-c4cccnc4)nc(Cl)c3c2)cc1] | 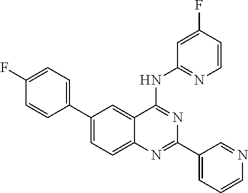 [SMILES: Fc1ccc(-c2ccc3nc(-c4cccnc4)nc(Nc4cc(F)ccn4)c3c2)cc1] | | 1H NMR (400 MHz, DMSO) δ 11.14 (s, 1H), 9.59-9.56 (m, 1H), 9.12 (d, J = 1.8 Hz, 1H), 8.76- 8.70 (m, 2H), 8.56-8.46 (m, 2H), 8.30 (dd, J = 8.8, 1.9 Hz, 1H), 8.10-8.04 (m, 2H), 8.01 (d, J = 8.7 Hz, 1H), 7.64- | DMSO | >98 | Method R4 F3 H1 | | | |
| | | | | | 7.59 (m, 1H), | | | | | | |
| | | | | | 7.44-7.36 (m, 2H), | | | | | | |
| | | | | | 7.18 (ddd, J = 8.1, | | | | | | |
| | | | | | 5.7, 2.4 Hz, 1H). | | | | | | |
| 443 | 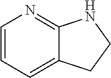 | 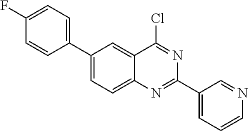 [SMILES: Fc1ccc(-c2ccc3nc(-c4cccnc4)nc(Cl)c3c2)cc1] | 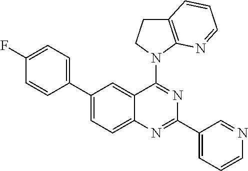 [SMILES: Fc1ccc(-c2ccc3nc(-c4cccnc4)nc(N4CCc5cccnc54)c3c2)cc1] | HCl | 1H NMR (400 MHz, DMSO) δ 9.70 (s, 1H), 9.03-8.96 (m, 1H), 8.87- 8.79 (m, 1H), 8.34 (d, J = 2.0 Hz, 1H), 8.27 (dd, J = 8.8, 2.1 Hz, 1H), 8.09 (d, J = 8.8 Hz, 1H), 7.96 (d, J = 5.0 Hz, 1H), 7.80- | DMSO | >98 | Method R4 F3 H1 | | | |
| | | | | | 7.70 (m, 4H), | | | | | | |
| | | | | | 7.36-7.29 (m, 2H), | | | | | | |
| | | | | | 7.00 (dd, J = | | | | | | |
| | | | | | 7.3, 5.1 Hz, 1H), | | | | | | |
| | | | | | 4.55 (t, J = 7.9 Hz | | | | | | |
| | | | | | 2H), 3.29 (t, J = | | | | | | |
| | | | | | 7.8 Hz, 2H). | | | | | | |
| 444 | 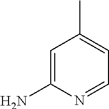 | 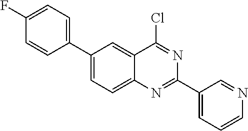 [SMILES: Fc1ccc(-c2ccc3nc(-c4cccnc4)nc(Cl)c3c2)cc1] | 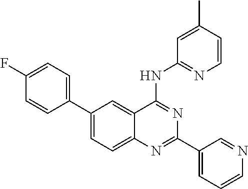 [SMILES: Cc1ccnc(Nc2nc(-c3cccnc3)nc3ccc(-c4ccc(F)cc4)cc23)c1] | 2HCl | 1H NMR (400 MHz, DMSO) δ 11.72 (s, 1H), 9.59 (s, 1H), 9.20 (s, 1H), 9.16 (d, J = 7.7 Hz, 1H), 8.98 (d, J = 5.0 Hz, 1H), 8.48 (d, J = 5.5 Hz, 1H), 8.41-8.35 (m, 2H), 8.14-8.00 (m, 4H), 7.44-7.36 (m, 2H), 7.31 (d, J = 5.5 Hz, 1H), | DMSO | >98 | Method R4 F3 H1 | | | |
| | | | | | 2.57 (s, 3H). | | | | | | |
| 445 | 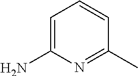 | 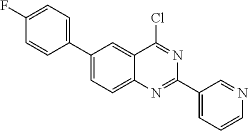 [SMILES: Fc1ccc(-c2ccc3nc(-c4cccnc4)nc(Cl)c3c2)cc1] | 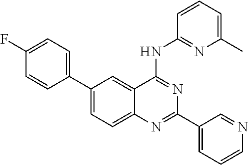 [SMILES: Cc1cccc(Nc2nc(-c3cccnc3)nc3ccc(-c4ccc(F)cc4)cc23)n1] | 2HCl | 1H NMR (400 MHz, DMSO) δ 11.46 (s, 1H), 9.60 (S, 1H), 9.20 (s, 1H), 9.14 (d, J = 7.6 Hz, 1H), 8.96 (d, J = 4.6 Hz, 1H), 8.42-8.30 (m, 2H), 8.16-7.96 (m, 5H), 7.47-7.35 (m, 2H), 7.25 (d, J = 7.6 Hz, 1H), 2.61 | DMSO | >98 | Method R4 F3 H1 | | | |
| | | | | | (s, 3H). | | | | | | |
| 446 | 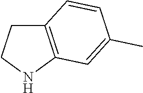 | 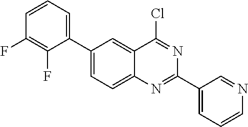 [SMILES: Fc1cccc(-c2ccc3nc(-c4cccnc4)nc(Cl)c3c2)c1F] | 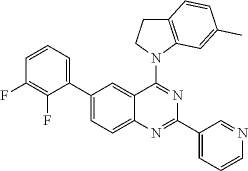 [SMILES: Cc1ccc2c(c1)N(c1nc(-c3cccnc3)nc3ccc(-c4cccc(F)c4F)cc13)CC2] | HCl | 1H NMR (400 MHz, DMSO) δ 9.58 (d, J = 1.6 Hz, 1H), 9.11 (d, J = 8.2 Hz, 1H), 8.95 (d, J = 4.1 Hz, 1H), 8.43 (d, J = 8.8 Hz, 1H), 8.21 (s, 1H), 8.07-7.96 (m, 1H), 7.86 (d, J = 8.8 Hz, 1H), 7.81 (s, | DMSO | >98 | Method G8 | | | |
| | | | | | 1H), 7.68-7.53 | | | | | | |
| | | | | | (m, 2H), 7.42 (dd, | | | | | | |
| | | | | | J = 13.0, 8.3 Hz, | | | | | | |
| | | | | | 1H), 7.29 (d, J = | | | | | | |
| | | | | | 7.6 Hz, 1H), 6.96 | | | | | | |
| | | | | | (d, J = 7.9 Hz, 1H), | | | | | | |
| | | | | | 4.70 (t, J = 7.9 | | | | | | |
| | | | | | Hz, 2H), 3.21 (t, J = | | | | | | |
| | | | | | 7.7 Hz, 2H), | | | | | | |
| | | | | | 2.40 (s, 3H). | | | | | | |

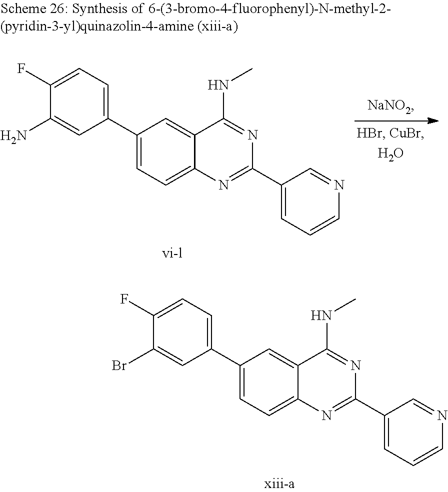 [SMILES: CNc1nc(-c2cccnc2)nc2ccc(-c3ccc(F)c(N)c3)cc12 ; CNc1nc(-c2cccnc2)nc2ccc(-c3ccc(F)c(Br)c3)cc12]

### Method T: 6-(3-bromo-4-fluorophenyl)-N-methyl-2-(pyridin-3-yl)quinazolin-4-amine 2HCl (xiii-a)

To a mixture of 6-(3-amino-4-fluorophenyl)-N-methyl-2-(pyridin-3-yl)quinazolin-4-amine (34.5 mg, 0.1 mmol) in HBr (48% solution in water, 2 mL) was added NaNO₂ (7 mg, 0.1 mmol) at 0° C. After the mixture was stirred at 0° C. for 20 min, CuBr (28 mg, 0 2 mmol) in HBr (1 mL of 48% solution in water) was added to the mixture. The resulting mixture was stirred at 0° C. and allowed to warm to room temperature and stirred for 18 h. The mixture was neutralized with Na₂CO₃ (aq.) and extracted with dichloromethane (3×100 mL). The combined organic layers were dried and concentrated to give a residue which was purified using Biotage Flash chromatography. The desired parent compound was dissolved in methanol, and 4N HCl in methanol (ca. 4 mL) was added to give a clear solution. The solution was concentrated to give 5.2 mg of HCl salt as a yellow solid with yield 10.4%. LCMS: retention time=1.822 min, [MH]⁺=408.9, 410.9. ¹H-NMR (400 MHz, DMSO-d₆): δ 9.86 (s, 1H), 9.47 (d, J=7.7 Hz, 1H), 9.17 (d, J=4.7 Hz, 1H), 8.74 (s, 1H), 8.40 (d, J=8.3 Hz, 1H), 8.32 (dd, J=7.1, 6.0 Hz, 1H), 8.20 (d, J=8.7 Hz, 1H), 8.17 (dd, J=6.5, 2.0 Hz, 1H), 7.89 (ddd, J=7.8, 4.2, 2.0 Hz, 1H), 7.43 (t, J=8.5 Hz, 1H), 3.51 (s, 3H).

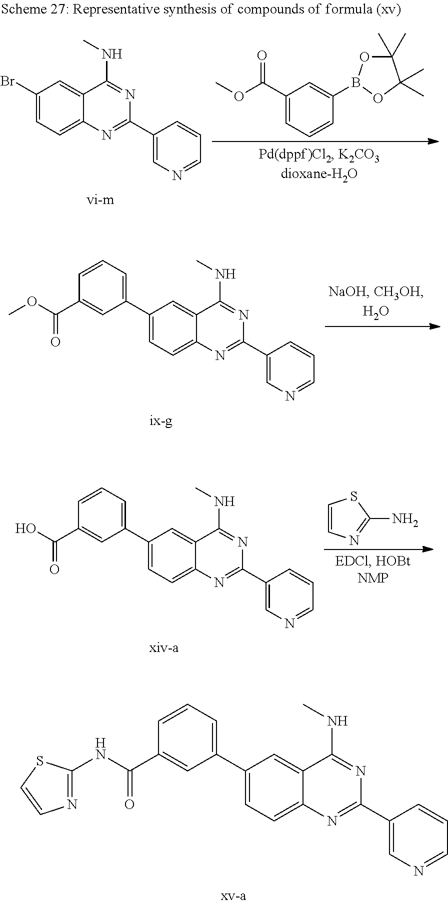

### Methyl 3-(4-(methylamino)-2-(pyridin-3-yl)quinazolin-6-yl)benzoate (ix-g)

A mixture of 6-bromo-N-methyl-2-(pyridin-3-yl)quinazolin-4-amine (5.30 g, 16.82 mmol), methyl 3-(4,4,5,5-tetramethyl-1,3,2-dioxaborolan-2-yl)benzoate (5.30 g, 20.22 mmol), Pd(dppf)Cl₂ (650 mg, 0.89 mmol) and K₂CO₃ (7.00 g, 50.64 mmol) were added to dioxane (350 ml) and refluxed overnight under a N₂ atmosphere. The volatiles were removed in vacuo and the residue was purified using silica gel chromatography using petroleum ether-ethyl acetate (1:1, and 3% TEA) to give methyl 3-(4-(methylamino)-2-(pyridin-3-yl)quinazolin-6-yl)benzoate (4.20 g, 67.4%). LCMS m/z=371 (M+1) (method B) (retention time=1.62 min)

### 3-(4-(methylamino)-2-(pyridin-3-yl)quinazolin-6-yl)benzoic acid (xiv-a)

To a solution of methyl 3-(4-(methylamino)-2-(pyridin-3-yl)quinazolin-6-yl)benzoate (4.20 g, 11.34 mmol) in methanol (200 ml) and water (20 ml) was added NaOH (1.40 g, 35.0 mmol). The mixture was stirred at 50° C. overnight. The volatiles were removed in vacuo and the residue was adjusted to pH 2 with 4N HCl. After filtration, 3-(4-(methylamino)-2-(pyridin-3-yl)quinazolin-6-yl)benzoic acid (3.26 g, 80.7%) was obtained. LCMS m/z=357 (M+1) (method B) (Retention time=1.25 min).

### Method U: 3-(4-(methylamino)-2-(pyridin-3-yl)quinazolin-6-yl)-N-(thiazol-2-yl)benzamide (xv-a)

A solution of 3-(4-(methylamino)-2-(pyridin-3-yl)quinazolin-6-yl)benzoic acid (700 mg, 1.96 mmol), EDCI (452 mg, 2.36 mmol) and HOBt (320 mg, 2.37 mmol) in NMP (15 ml) was stirred at room temperature for 1 h. Thiazol-2-amine (217 mg, 2.17 mmol) was added. The mixture was stirred at 60° C. overnight. 100 mL of water was added to the mixture and a precipitate formed. The solid was collected and purified using biotage chromatography to give 3-(4-(methylamino)-2-(pyridin-3-yl)quinazolin-6-yl)-N-(thiazol-2-yl)benzamide (133.9 mg, 15.6%). LCMS m/z=439 (M+1) (method B) (Retention time=1.64 min) ¹H NMR (400 MHz, DMSO) δ 12.84 (s, 1H), 9.67 (s, 1H), 8.80 (d, J=8.0 Hz, 1H), 8.70 (s, 3H), 8.62 (s, 1H), 8.33 (d, J=8.5 Hz, 1H), 8.12 (d, J=7.6 Hz, 2H), 7.92 (d, J=8.8 Hz, 1H), 7.72 (t, J=7.6 Hz, 1H), 7.59 (d, J=3.4 Hz, 1H), 7.56 (dd, J=7.8, 5.0 Hz, 1H), 7.30 (d, J=2.8 Hz, 1H), 3.21 (d, J=4.2 Hz, 3H).

The compounds in the following table were prepared in a manner analogous to that described in Scheme 27, replacing thiazol-2-amine with the appropriate amine

TABLE 7

| | Starting | Starting | |
|---|---|---|---|
| Number | Material 1 | Material 2 | Product |
| 447 | 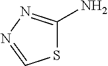 | 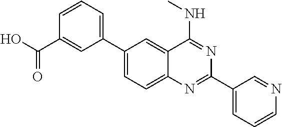 [SMILES: CNc1nc(-c2cccnc2)nc2ccc(-c3cccc(C(=O)O)c3)cc12] | 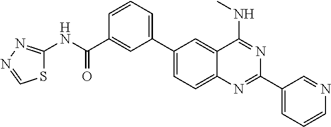 [SMILES: CNc1nc(-c2cccnc2)nc2ccc(-c3cccc(C(=O)Nc4nncs4)c3)cc12] |
| 448 | MeOH | 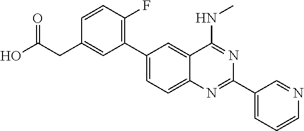 [SMILES: CNc1nc(-c2cccnc2)nc2ccc(-c3cc(CC(=O)O)ccc3F)cc12] | 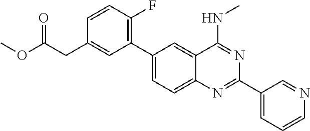 [SMILES: CNc1nc(-c2cccnc2)nc2ccc(-c3cc(CC(=O)OC)ccc3F)cc12] |

| | Salt | | 1H NMR | Purity | Method | | Retention | LCMS |
|---|---|---|---|---|---|---|---|---|
| Number | Type | ¹H NMR | Solvent | percent | of Coupling | LCMS | Time | Method |
| 447 | 2HCl | ¹H-NMR (400 MHz, DMSO-d₆): | DMSO | 95 | Method U | 440.0 | t = 1.509 | Method B |
| | | 13.24 (s, 1H), 10.33 (s, 1H), 9.68 (s, | | | | (M + 1) | min | (NH4HCO3) |
| | | 1H), 9.29 (s, 1H), 9.09 (d, J = 7.5 | | | | | | |
| | | Hz, 1H), 9.02 (s, 1H), 8.97 (d, J = | | | | | | |
| | | 4.8 Hz, 1H), 8.66 (s, 1H), 8.52 (d, | | | | | | |
| | | J = 8.5 Hz, 1H), 8.29 (d, J = 8.6 Hz, | | | | | | |
| | | 1H), 8.23 (d, J = 7.7 Hz, 1H), 8.18 | | | | | | |
| | | (d, J = 7.8 Hz, 1H), 7.91 (dd, J = | | | | | | |
| | | 7.5, 5.3 Hz, 1H), 7.76 (t, J = 7.7 Hz, | | | | | | |
| | | 1H), 3.33 (d, J = 4.3 Hz, 3H). | | | | | | |
| 448 | | 1H-NMR (400 MHz, DMSO-d6): δ | DMSO | 95 | Method U | 402.9 | t = 1.612 | Method A |
| | | 9.63 (s, 1H), 9.42 (s, 1H), 8.90 (s, | | | | (M + 1) | min | (TFA) |
| | | 2H), 8.54 (s, 1H), 8.08 (d, J = 8.4 | | | | | | |
| | | Hz, 1H), 8.90 (d, J = 8.8 Hz, 1H), | | | | | | |
| | | 7.78 (s, 1H), 7.56 (d, J = 7.6 Hz, | | | | | | |
| | | 1H), 7.36-7.43 (m, 2H), 3.81 (s, | | | | | | |
| | | 2H), 3.60 (s, 3H), 3.24 (s, 3H) | | | | | | |

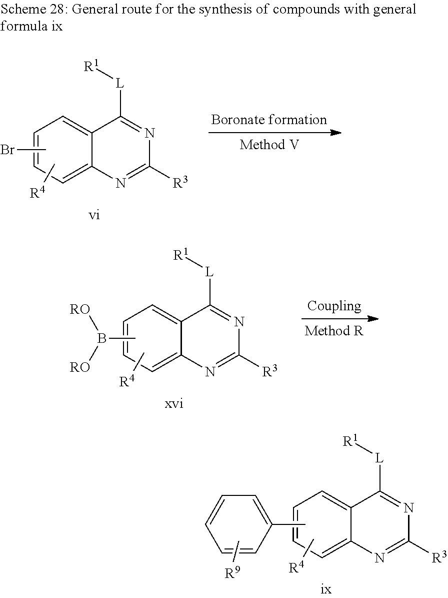

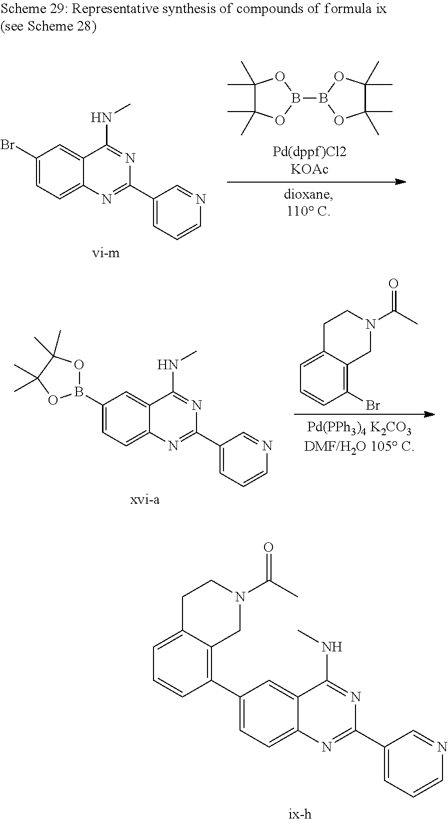

### Method V: N-methyl-2-(pyridin-3-yl)-6-(4,4,5,5-tetramethyl-1,3,2-dioxaborolan-2-yl) quinazolin-4-amine (xvi-a)

A flask was charged with 6-bromo-N-methyl-2-(pyridin-3-yl)quinazolin-4-amine (5.00 g, 15.86 mmol), bis(pinacolato)diboron (8.05 g, 31.72 mmol, 2.0 equiv), Pd(dppf)Cl₂ (1.29 g, 1.58 mmol, 10 mol %) and potassium acetate (6.22 g, 63.45 mmol, 4.0 equiv). The mixture was suspended in dioxane (350 mL) and the reaction was heated under an argon atmosphere at 110° C. overnight. After cooling, the volatiles were removed in vacuo. The residue was purified by chromatography (silica gel, petroleum ether: ethyl acetate from 100:1). N-methyl-2-(pyridin-3-yl)-6-(4,4,5,5-tetramethyl-1,3,2-dioxaborolan-2-yl) quinazolin-4-amine (3.33 g, 58% yield) was obtained as a light yellow solid. LCMS m/z=363.1 (M+1) (Method B) (retention time=1.83 min) ¹H NMR (400 MHz, CDCl₃) δ 9.82 (s, 1H), 8.85 (d, J=8.0 Hz, 1H), 8.74 (s, 1H), 8.21 (s, 1H), 8.12 (d, J=8.8 Hz, 1H), 7.88 (d, J=8.4 Hz, 1H), 7.43 (s, 1H), 6.06 (s, 1H), 3.32 (d, J=4.8 Hz, 3H), 1.38 (s, 12H).

### Method R3: 1-(8-(4-(methylamino)-2-(pyridin-3-yl)quinazolin-6-yl)-3,4-dihydroisoquinolin-2(1H)-yl)ethanone (ix-h)

A 25 ml reaction flask was charged with N-methyl-2-(pyridin-3-yl)-6-(4,4,5,5-tetramethyl-1,3,2-dioxaborolan-2-yl)quinazolin-4-amine (100 mg, 0.276 mmol, 1.0 equiv), 1-(8-bromo-3,4-dihydroisoquinolin-2(1H)-yl)ethanone (70.2 mg, 0.276 mmol, 1.0 equiv), Pd(PPh₃)₄ (12.7 mg, 0.011 mmol, 4 mol %) and K₂CO₃ (114.5 mg, 0.828 mmol, 3.0 equiv). The mixture was suspended in DMF/H₂O (20:1, 6 mL), and the reaction was heated at 105° C. for 4 h. After cooling, the reaction was diluted with water (30 mL) and the resultant precipitate was collected by filtration. The crude product was purified on reverse phase HPLC (50% MeCN:H₂O, Rt=15 min) to give the desired product as a yellow solid (50 mg, 44%). LCMS m/z=410.2 (M+1) (Method B) (retention time=1.72 min) ¹H NMR (300 MHz, DMSO-d6): δ 9.67 (s, 1H), 8.81-8.68 (m, 2H), 8.29-8.21 (m, 2H), 7.89-7.75 (m, 2H), 7.56-7.51 (m, 1H), 7.35-7.22 (m, 3H), 4.55 (s, 2H), 3.72-3.68 (m, 2H), 3.20-3.18 (m, 3H), 3.05-2.96 (m, 2H), 2.02 (brs, 3H).

### Method R7: 5-(4-(methylamino)-2-(pyridin-3-yl)quinazolin-6-yl)isoindolin-1-one, 2 HCl (ix-m)

To a 10 mL microwave vial were added N-methyl-2-(pyridin-3-yl)-6-(4,4,5,5-tetramethyl-1,3,2-dioxaborolan-2-yl)quinazolin-4-amine (0.225 g, 0.621 mmol), 5-bromoisoindolin-1-one (0.120 g, 0.565 mmol), trans-dichlorobis(triphenylphosphine)palladium (II) (Pd(PPh₃)₂Cl₂, 0.020 g, 0.028 mmol) and potassium carbonate (0.390 g, 2.82 mmol) in DME (1.5 ml)/water (0.429 ml)/ethanol (0.643 ml) to give a brown suspension. The reaction mixture was then heated to 120° C. for 20 min by microwave irradiation. LC-MS analysis of the crude mixture showed the reaction was completed. The reaction mixture was washed with water (40 mL) to yield a tan precipitate. The precipitate was purified via ISCO (silica gel, 93:7 CH₂Cl₂/MeOH, 12 gm column). The fractions collected were concentrated and dried under vacuum to give a tan solid. To form the salt, the material was suspended in methanol prior to the addition of 4 M HCl in dioxane. After stirring at ambient temperature for 2 h, the solvent was evaporated to give the desired product as a yellow solid (116.5 mg, 0.26 mmol, 47%). LC-MS m/z=368.2 (M+1) (retention time=1.61) ¹H NMR (300 MHz, DMSO) δ 10.19 (s, 1H), 9.63 (d, J=1.4 Hz, 1H), 9.02 (d, J=7.0 Hz, 1H), 8.98-8.86 (m, 2H), 8.69 (s, 1H), 8.39 (d, J=8.4 Hz, 1H), 8.20 (d, J=8.7 Hz, 1H), 8.06 (s, 1H), 7.98 (d, J=8.1 Hz, 1H), 7.92-7.76 (m, 2H), 4.47 (s, 2H), 3.30 (d, J=4.2 Hz, 3H).

### Method R2: N-(2-methoxy-5-(4-(methylamino)-2-(pyridin-3-yl)quinazolin-6-yl)phenyl)acetamide (ix-n)

To a 20 mL reaction vial were added N-methyl-2-(pyridin-3-yl)-6-(4,4,5,5-tetramethyl-1,3,2-dioxaborolan-2-yl)quinazolin-4-amine (0.2 g, 0.552 mmol), N-(5-bromo-2-methoxyphenyl)acetamide (0.162 g, 0.663 mmol), bis(di-tert-butyl(4-dimethylaminophenyephosphine)dichloropalladium(II) (0.012 g, 0.017 mmol) and potassium phosphate tribasic monohydrate (0.381 g, 1.656 mmol) in dioxane (5 ml)/water (0.5 ml) to give a tan suspension. The reaction was heated at 90° C. overnight. LC-MS analysis of the crude mixture showed the reaction was completed. The reaction mixture was washed with water (40 mL), and the precipitate was collected as a brown solid. The precipitate was purified via ISCO (silica gel, 96:4 CH₂Cl₂/MeOH, 12 gm column). The fractions collected were concentrated and dried under vacuum to yield the title compound as an off-white powder (101.4 mg, 0.25 mmol, 46%). LC-MS m/z=400.3 (M+1) (retention time=1.83) ¹H NMR (300 MHz, DMSO) δ 9.62 (d, J=1.2 Hz, 1H), 9.31 (s, 1H), 8.76 (dd, J=9.8, 1.8 Hz, 1H), 8.66 (dd, J=4.7, 1.7 Hz, 1H), 8.61 (d, J=4.6 Hz, 1H), 8.46 (s, 1H), 8.34 (s, 1H), 8.00 (d, J=8.8 Hz, 1H), 7.83 (d, J=8.7 Hz, 1H), 7.60-7.47 (m, 2H), 7.18 (d, J=8.6 Hz, 1H), 3.89 (s, 3H), 3.16 (d, J=4.3 Hz, 4H), 2.11 (s, 3H).

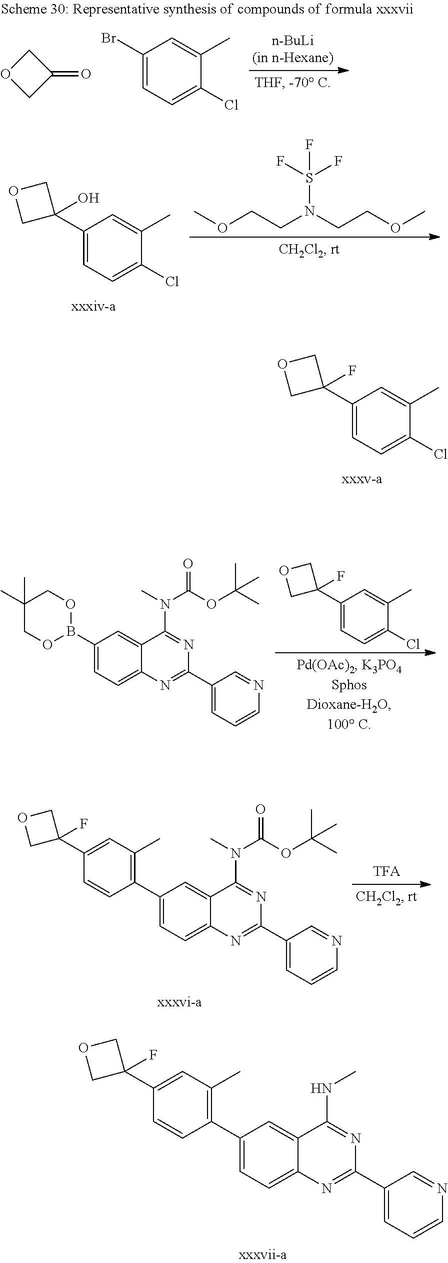

### 3-(4-Chloro-3-methylphenyl)oxetan-3-ol (xxxiv-a)

To a solution of 5-bromo-2-chlorotoluene (1.56 g, 7.63 mmol) in THF (50 mL) was added n-butyl lithium (2.66 mol/L in n-hexane, 2.61 mL, 6.94 mmol) at −70° C. After stirring at −70° C. for 2 h, 3-oxetanone (0.50 g, 6.94 mmol) was added to the reaction and stirring was continued for an additional 2 h at −70° C. After the reaction was completed, water was added at room temperature and extracted with ethyl acetate (50 mL×2). The organic extracts were combined, washed with brine, dried over MgSO₄, filtered and concentrated. The crude product was purified via ISCO (silica gel, hexane/ethyl acetate=10/1-2/1) to afford 1.37 g (99% yield) of 3-(4-chloro-3-methylphenyl)oxetan-3-ol as a white powder. ¹H NMR (400 MHz, CDCl₃) δ 7.51-7.44 (m, 1H), 7.43-7.30 (m, 2H), 5.02-4.78 (m, 4H), 2.61 (s, 1H), 2.41 (s, 3H).

### 3-(4-Chloro-3-methylphenyl)-3-fluorooxetane (xxxv-a)

To a solution of 3-(4-chloro-3-methylphenyl)oxetan-3-ol (400 mg, 2.01 mmol) in CH₂Cl₂ (5 mL) was added bis(2-methoxyethyl)aminosulfur trifluoride (891 mg, 4.03 mmol) at 0° C. The reaction was stirred at room temperature for 20 h. After the reaction was completed, NH₄Cl aqueous solution was added to quench the reaction, and then extracted with ethyl acetate (50 mL×2). The organic extracts were combined, washed with brine, dried over MgSO₄, filtered and concentrated. The crude product was purified via ISCO (silica gel, hexane/ethyl acetate=10/1) to afford 342 mg (84% yield) of the 3-(4-chloro-3-methylphenyl)-3-fluorooxetane as a colorless oil. ¹H NMR (400 MHz, CDCl3) δ 7.42 (d, J=1.8 Hz, 1H), 7.40 (d, J=8.3 Hz, 1H), 7.32 (dd, J=8.3, 2.3 Hz, 1H), 5.17-5.03 (m, 2H), 4.89-4.75 (m, 2H), 2.42 (s, 3H).

### Method R5: tert-Butyl 6-(4-(3-fluorooxetan-3-yl)-2-methylphenyl)-2-(pyridin-3-yl)quinazolin-4-ylmethyl)carbamate (xxxvi-a)

A mixture of the boron ester quinazoline derivative (400 mg, 0.892 mmol), 3-(4-chloro-3-methylphenyl)-3-fluorooxetane (215 mg, 1.07 mmol), Pd(OAc)₂ (20 mg, 0.089 mmol), Sphos (110 mg, 0.268 mmol), K₃PO₄ (568 mg, 2.68 mmol) were added to dioxane (15 ml) and water (3 ml) and stirred at 100° C. under N₂ atmosphere for 3 h. After cooling to room temperature, water was added and extracted with ethyl acetate (50 mL×2), washed with brine, dried over MgSO₄, filtered and concentrated. The crude product was purified via ISCO(NH-silica gel, hexane/ethyl acetate=10/1-3/1) to afford 246 mg (55% yield) of the desired product as a colorless oil. ¹H NMR (400 MHz, CDCl₃) δ 9.81 (dd, J=2.2, 0.8 Hz, 1H), 8.90-8.83 (m, 1H), 8.75 (dd, J=4.8, 1.7 Hz, 1H), 8.13 (dd, J=7.8, 1.5 Hz, 1H), 7.90-7.81 (m, 2H), 7.55-7.42 (m, 3H), 7.39 (d, J=7.9 Hz, 1H), 5.24-5.10 (m, 2H), 5.00-4.85 (m, 2H), 3.58 (s, 3H), 2.37 (s, 3H), 1.36 (s, 9H).

### 6-(4-(3-Fluorooxetan-3-yl)-2-methylphenyl)-N-methyl-2-(pyridin-3-yl)quinazolin-4-amine (xxxvii-a)

To a suspension of the tert-butyl 6-(4-(3-fluorooxetan-3-yl)-2-methylphenyl)-2-(pyridin-3-yl)quinazolin-4-yl(methyl)carbamate (235 mg, 0.469 mmol) in dichloromethane (3 ml) was added trifluoroacetic acid (1 ml). The reaction was stirred at room temperature for 3 h. After the reaction was completed, the volatiles were evaporated. To the residue was added water and neutralized with aqueous NaOH solution. The product was extracted with ethyl acetate (50 mL×2), washed with brine, dried over MgSO₄, filtered and concentrated. The crude product was dissolved in ethanol, and NH-silica gel was added and concentrated. The silica gel was charged onto the ISCO column for purification (ISCO, NH-silica gel, hexane/ethyl acetate=10/1-1/1) to give 101 mg (53% yield) of the desired product as a white powder. ¹H NMR 400 MHz, DMSO) δ 9.65 (dd, J=2.1, 0.8 Hz, 1H), 8.83-8.74 (m, 1H), 8.69 (dd, J=4.8, 1.7 Hz, 1H), 8.55-8.45 (m, 1H), 8.25 (d, J=1.6 Hz, 1H), 7.86 (d, J=8.5 Hz, 1H), 7.81 (dd, J=8.5, 1.8 Hz, 1H), 7.62-7.53 (m, 2H), 7.51 (d, J=8.0 Hz, 1H), 7.44 (d, J=7.9 Hz, 1H), 5.09-4.92 (m, 4H), 3.16 (d, J=4.5 Hz, 3H), 2.36 (s, 3H).

The compound in the following table was prepared in a manner analogous to that described in Scheme 29 and 30.

TABLE 8

| Num- | Starting | Starting | | Salt | |
|---|---|---|---|---|---|
| ber | Material 1 | Material 2 | Product | type | ¹H NMR |
| 449 | 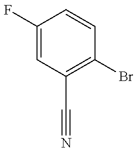 | 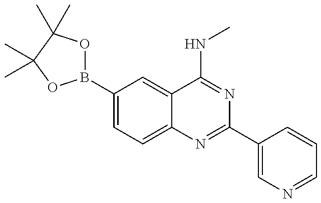 [SMILES: CNc1nc(-c2cccnc2)nc2ccc(B3OC(C)(C)C(C)(C)O3)cc12] | 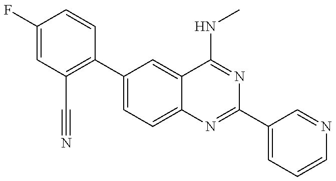 [SMILES: CNc1nc(-c2cccnc2)nc2ccc(-c3ccc(F)cc3C#N)cc12] | HCl | ¹H-NMR (400 MHz, DMSO-d₆): δ 9.66 (s, 2H), 9.03 (d, J = 7.9 Hz, 1H), 8.91 (d, J = 4.3 Hz, 1H), 8.74 (s, 1H), 8.59 (s, 1H), 8.28 (d, J = 8.3 Hz, 1H), 8.22 (d, J = 8.6 Hz, 1H), 8.14 (d, J = 8.5 Hz, 1H), 8.02 (d, J = 8.2 Hz, 1H), 7.85 (dd, J = 7.8, 5.1 Hz, 1H), 3.26 (d, J = 4.2 Hz, 3H). |
| 450 | 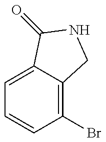 [SMILES: O=C1NCc2c(Br)cccc21] | 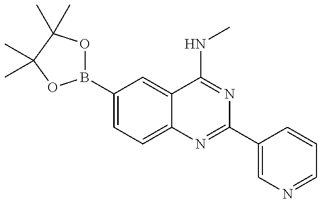 [SMILES: CNc1nc(-c2cccnc2)nc2ccc(B3OC(C)(C)C(C)(C)O3)cc12] | 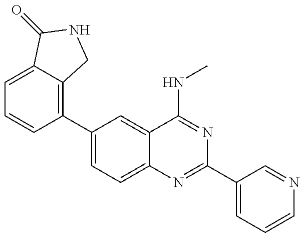 [SMILES: CNc1nc(-c2cccnc2)nc2ccc(-c3cccc4c3CNC4=O)cc12] | HCl | ¹H-NMR (400 MHz, DMSO-d₆): δ 9.66 (d, J = 1.7 Hz, 1H), 9.05 (s, 1H), 8.96 (dd, J = 5.0, 1.2 Hz, 1H), 8.82 (s, 1H), 8.74 (s, 1H), 8.29-8.19 (m, 2H), 7.93- 7.85 (m, 1H), 7.82 (d, J = 7.4 Hz, 1H), 7.79 (d, J = 7.2 Hz, 1H), 7.69 (t, J = 7.5 Hz, 1H), 4.69 (s, 2H), 3.30 (d, J = 4.4 Hz, 3H). |
| 451 | 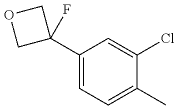 | 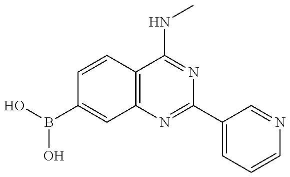 [SMILES: CNc1nc(-c2cccnc2)nc2cc(B(O)O)ccc12] | 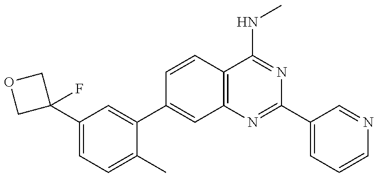 [SMILES: CNc1nc(-c2cccnc2)nc2cc(-c3cc(C4(F)COC4)ccc3C)ccc12] | | 1H NMR (400 MHz, DMSO) δ 9.66 (dd, J = 2.1, 0.7 Hz, 1H), 8.82-8.75 (m, 1H), 8.69 (dd, J = 4.7, 1.7 Hz, 1H), 8.58-8.50 (m, 1H), 8.30 (d, J = 8.5 Hz, 1H), 7.75 (d, J = 1.7 Hz, 1H), 7.60-7.42 (m, 5H), 5.12-4.91 (m, 4H), 3.19 (d, J = 4.5 Hz, 3H), 2.33 (s, 3H). |
| 452 | 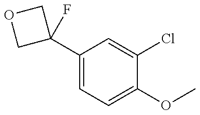 | 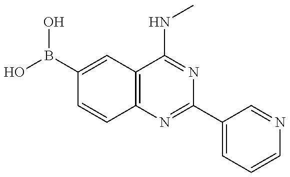 [SMILES: CNc1nc(-c2cccnc2)nc2ccc(B(O)O)cc12] | 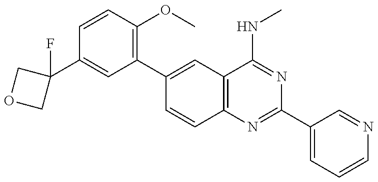 [SMILES: CNc1nc(-c2cccnc2)nc2ccc(-c3cc(C4(F)COC4)ccc3OC)cc12] | | 1H NMR (400 MHz, DMSO) δ 9.66 (dd, J = 2.1, 0.7 Hz, 1H), 8.84- 8.76 (m, 1H), 8.69 (dd, J = 4.8, 1.7 Hz, 1H), 8.56-8.45 (m, 1H), 8.33 (d, J = 1.7 Hz, 1H), 7.96 (dd, J = 8.6, 1.9 Hz, 1H), 7.81 (d, J = 8.6 Hz, 1H), 7.61-7.52 (m, 3H), 7.27 (d, J = 8.4 Hz, 1H), 5.13-4.93 (m, 4H), 3.85 (s, 3H), 3.17 (d, J = 4.5 Hz, 3H). |
| 453 | 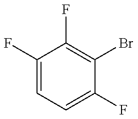 | 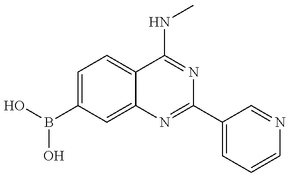 [SMILES: CNc1nc(-c2cccnc2)nc2cc(B(O)O)ccc12] | 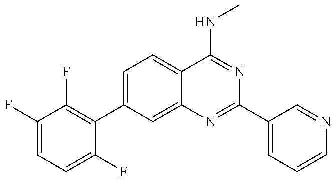 [SMILES: CNc1nc(-c2cccnc2)nc2cc(-c3c(F)ccc(F)c3F)ccc12] | | 1H NMR (400 MHz, DMSO) δ 9.65 (d, J = 1.5 Hz, 1H), 8.82-8.73 (m, 1H), 8.70 (dd, J = 4.7, 1.6 Hz, 1H), 8.66-8.57 (m, 1H), 8.36 (d, J = 8.5 Hz, 1H), 7.91 (s, 1H), 7.71-7.58 (m, 2H), 7.55 (dd, J = 7.9, 4.8 Hz, 1H), 7.41-7.28 (m, 1H), 3.19 (d, J = 4.5 Hz, 3H). |
| 454 | 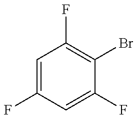 | 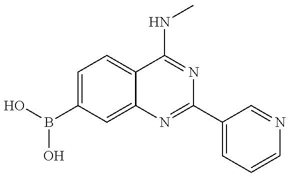 [SMILES: CNc1nc(-c2cccnc2)nc2cc(B(O)O)ccc12] | 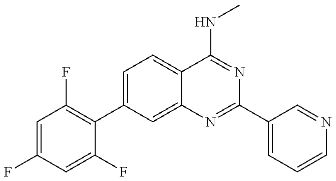 [SMILES: CNc1nc(-c2cccnc2)nc2cc(-c3c(F)cc(F)cc3F)ccc12] | HCl | 1H NMR (400 MHz, DMSO) δ 10.23 (brs, 1H), 9.69 (d, J = 1.7 Hz, 1H), 9.13 (d, J = 7.6 Hz, 1H), 8.97 (dd, J = 5.1, 1.3 Hz, 1H), 8.65 (d, J = 8.6 Hz, 1H), 8.29 (s, 1H), 7.92 (dd, J = 7.9, 5.2 Hz, 1H), 7.81 (d, J = 8.4 Hz, 1H), 7.52-7.38 (m, 2H), 3.30 (d, J = 4.5 Hz, 3H). |
| 455 | 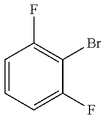 | 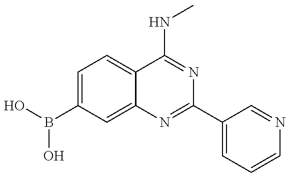 [SMILES: CNc1nc(-c2cccnc2)nc2cc(B(O)O)ccc12] | 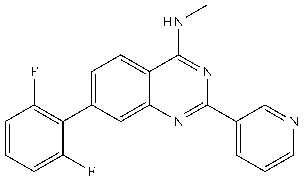 [SMILES: CNc1nc(-c2cccnc2)nc2cc(-c3c(F)cccc3F)ccc12] | 2 HCl | 1H NMR (400 MHz, DMSO) δ 10.46 (brs, 1H), 9.76-9.71 (m, 1H), 9.22 (d, J = 8.2 Hz, 1H), 9.06- 8.96 (m, 1H), 8.72 (d, J = 8.6 Hz, 1H), 8.40 (s, 1H), 7.97 (dd, J = 7.9, 5.4 Hz, 1H), 7.85 (d, J = 8.5 Hz, 1H), 7.69-7.54 (m, 1H), 7.45-7.28 (m, 2H), 3.31 (d, J = 4.3 Hz, 3H). |
| 456 | 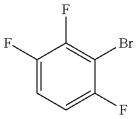 | 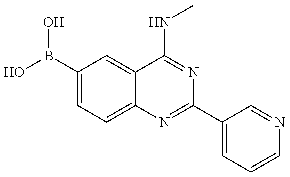 [SMILES: CNc1nc(-c2cccnc2)nc2ccc(B(O)O)cc12] | 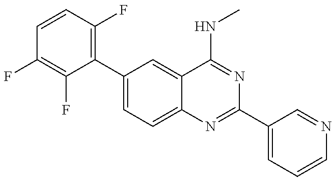 [SMILES: CNc1nc(-c2cccnc2)nc2ccc(-c3c(F)ccc(F)c3F)cc12] | | 1H NMR (400 MHz, DMSO) δ 9.71-9.60 (m ,1H), 8.83-8.76 (m, 1H), 8.70 (dd, J = 4.8, 1.7 Hz, 1H), 8.66-8.56 (m, 1H), 8.44 (s, 1H), 7.95-7.85 (m, 2H), 7.63 (ddd, J = 19.1, 9.4, 5.0 Hz, 1H), 7.56 (ddd, J = 8.0, 4.8, 0.7 Hz, 1H), 7.41-7.29 (m, 1H), 3.17 (d, J = 4.5 Hz, 3H). |
| 457 | 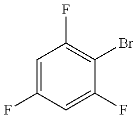 | 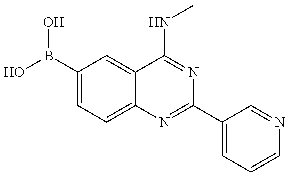 [SMILES: CNc1nc(-c2cccnc2)nc2ccc(B(O)O)cc12] | 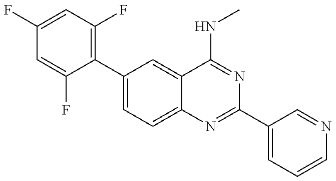 [SMILES: CNc1nc(-c2cccnc2)nc2ccc(-c3c(F)cc(F)cc3F)cc12] | HCl | 1H NMR (400 MHz, DMSO) δ 9.64 (s, 2H), 9.09-8.96 (m, 1H), 8.93 (d, J = 4.1 Hz, 1H), 8.56 (s, 1H), 8.12 (d, J = 8.2 Hz, 1H), 8.03 (d, J = 8.2 Hz, 1H), 7.91-7.79 (m, 1H), 7.56-7.39 (m, 2H), 3.26 (d, J = 4.1 Hz, 3H). |
| 458 | 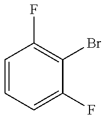 | 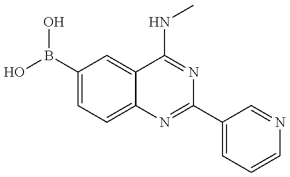 [SMILES: CNc1nc(-c2cccnc2)nc2ccc(B(O)O)cc12] | 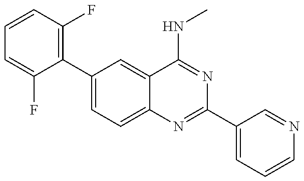 [SMILES: CNc1nc(-c2cccnc2)nc2ccc(-c3c(F)cccc3F)cc12] | 2 HCl | 1H NMR (400 MHz, DMSO) δ 10.02 brs, 1H), 9.69 (s, 1H), 9.18-9.03 (m, 1H), 8.94 (d, J = 5.0 Hz, 1H), 8.68 (s, 1H), 8.42-8.23 (m, 1H), 8.06 (d, J = 8.3 Hz, 1H), 7.92-7.79 (m, 1H), 7.69-7.53 (m, 1H), 7.42- 7.17 (m, 2H), 3.27 (d, J = 4.4 Hz, 3H). |
| 459 | 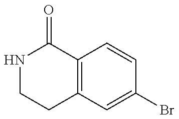 [SMILES: O=C1NCCc2cc(Br)ccc21] | 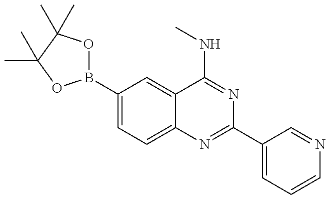 [SMILES: CNc1nc(-c2cccnc2)nc2ccc(B3OC(C)(C)C(C)(C)O3)cc12] | 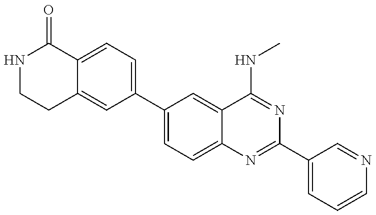 [SMILES: CNc1nc(-c2cccnc2)nc2ccc(-c3ccc4c(c3)CCNC4=O)cc12] | 2 HCl | 1H NMR (300 MHz, DMSO) δ 10.08 (s, 1H), 9.62 (d, J = 1.1 Hz, 1H), 8.99 (d, J = 6.1 Hz, 1H), 8.93 (dd, O J = 4.9, 1.4 Hz, 1H), 8.88 (d, J = 1.7 Hz, 1H), 8.38 (dd, J = 9.2, 1.2 Hz, 1H), 8.14 (d, J = 9.0 Hz, 1H), 8.04 (s, 1H), 7.98 (d, J = 8.6 Hz, 1H), 7.92-7.80 (m, 3H), 3.43 (s, 2H), 3.30 (d, J = 4.2 Hz, 3H), 3.01 (t, J = 6.1 Hz, 2H). |
| 460 | 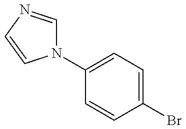 [SMILES: Brc1ccc(-n2ccnc2)cc1] | 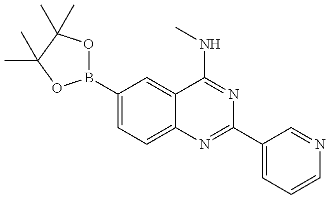 [SMILES: CNc1nc(-c2cccnc2)nc2ccc(B3OC(C)(C)C(C)(C)O3)cc12] | 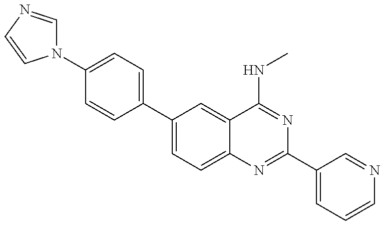 [SMILES: CNc1nc(-c2cccnc2)nc2ccc(-c3ccc(-n4ccnc4)cc3)cc12] | 2 HCl | 1H NMR (300 MHz, DMSO) δ 10.56 (s, 1H), 9.87 (s, 1H), 9.66 (d, J = 1.8 Hz, 1H), 9.15 (s, 1H), 9.06 (d, J = 8.9 Hz, 1H), 8.93 (d, J = 3.5 Hz, 1H), 8.52-8.39 (m, 2H), 8.33-8.18 (m, 3H), 8.08- 7.94 (m, 3H), 7.86 (dd, J = 7.6, 4.6 Hz, 1H), 3.30 (d, J = 4.3 Hz, 3H) |
| 461 | 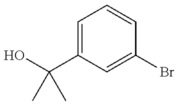 | 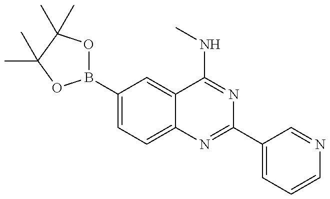 [SMILES: CNc1nc(-c2cccnc2)nc2ccc(B3OC(C)(C)C(C)(C)O3)cc12] | 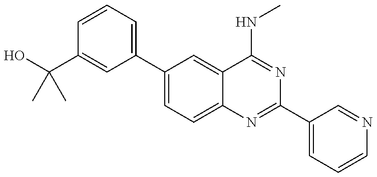 [SMILES: CNc1nc(-c2cccnc2)nc2ccc(-c3cccc(C(C)(C)O)c3)cc12] | 2 HCl | 1H NMR (300 MHz, DMSO) δ 10.36 (s, 1H), 9.63 (d, J = 1.8 Hz, 1H), 9.00 (d, J = 6.4 Hz, 1H), 8.94 (dd, J = 5.0, 1.5 Hz, 1H), 8.86 (s, 1H), 8.37 (d, J = 1.5 Hz, 1H), 8.20 (d, J = 8.3 Hz, 1H), 7.97 (s, 1H), 7.86 (dd, J = 7.3, 4.5 Hz, 1H), 7.70 (d, J = 7.5 Hz, 1H), 7.55 (d, J = 7.8 Hz, 1H), 7.49 (d, J = 7.6 Hz, 1H), 3.31 (d, J = 4.4 Hz, 3H), 1.51 (s, J = 9.8 Hz, 6H). |
| 462 | 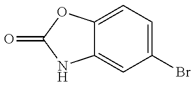 [SMILES: O=c1[nH]c2cc(Br)ccc2o1] | 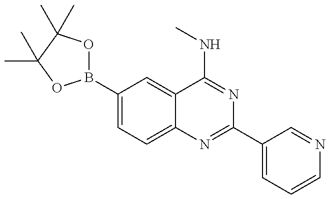 [SMILES: CNc1nc(-c2cccnc2)nc2ccc(B3OC(C)(C)C(C)(C)O3)cc12] | 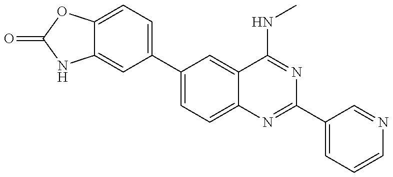 [SMILES: CNc1nc(-c2cccnc2)nc2ccc(-c3ccc4oc(=O)[nH]c4c3)cc12] | 2 HCl | 1H NMR (300 MHz, DMSO) δ 11.99 (s, 1H), 9.60 (d, J = 1.4 Hz, 1H), 9.00-8.87 (m, 2H), 8.75 (s, 1H), 8.31 (d, J = 10.2 Hz, 1H), 8.08 (d, J = 7.2 Hz, 1H), 7.83 (dd, J = 8.4, 6.1 Hz, 1H), 7.57 (dd, J = 7.8, 1.0 Hz, 1H), 7.54-7.44 (m, 2H), 3.29 (d, J = 4.2 Hz, 3H). |
| 463 | 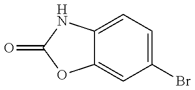 [SMILES: O=c1[nH]c2ccc(Br)cc2o1] | 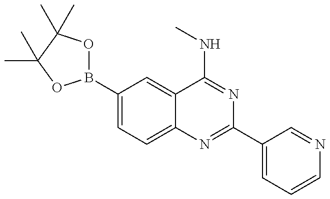 [SMILES: CNc1nc(-c2cccnc2)nc2ccc(B3OC(C)(C)C(C)(C)O3)cc12] | 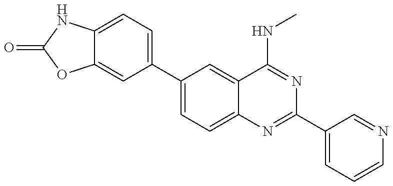 [SMILES: CNc1nc(-c2cccnc2)nc2ccc(-c3ccc4[nH]c(=O)oc4c3)cc12] | 2 HCl | 1H NMR (300 MHz, DMSO) δ 11.89 (s, 1H), 10.22 (s, 1H), 9.61 (s, 1H), 9.05-8.88 (m, 2H), 8.82 (s, 1H), 8.34 (d, J = 8.7 Hz, 1H), 8.15 (d, J = 8.5 Hz, 1H), 7.84 (s, 2H), 7.69 (d, J = 8.7 Hz, 1H), 7.23 (d, J = 8.2 Hz, 1H), 3.29 (d, J = 3.9 Hz, 3H). |
| 464 | 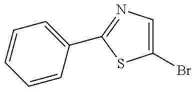 [SMILES: Brc1cnc(-c2ccccc2)s1] | 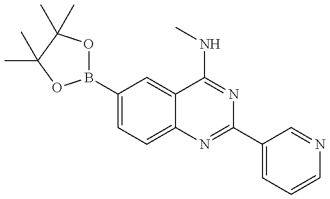 [SMILES: CNc1nc(-c2cccnc2)nc2ccc(B3OC(C)(C)C(C)(C)O3)cc12] | 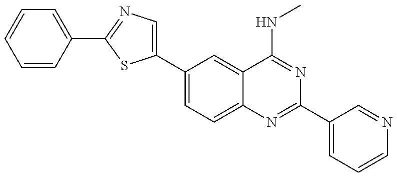 [SMILES: CNc1nc(-c2cccnc2)nc2ccc(-c3cnc(-c4ccccc4)s3)cc12] | 2 HCl | 1H NMR (300 MHz, DMSO) δ 10.07 (s, 1H), 9.61 (d, J = 1.6 Hz, 1H), 9.04 (d, J = 5.6 Hz, 1H), 8.93 (dd, J = 5.1, 1.4 Hz, 1H), 8.86 (d, J = 0.5 Hz, 1H), 8.54 (s, 1H), 8.31 (dd, J = 8.7, 1.5 Hz, 1H), 8.10 (d, J = 8.7 Hz, 1H), 7.96 (dd, J = 7.5, 2.1 Hz, 2H), 7.88 (dd, J = 7.3, 4.5 Hz, 1H), 7.60-7.48 (m, 3H), 3.28 (d, J = 4.2 Hz, 3H). |
| 465 | 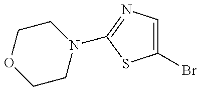 [SMILES: Brc1cnc(N2CCOCC2)s1] | 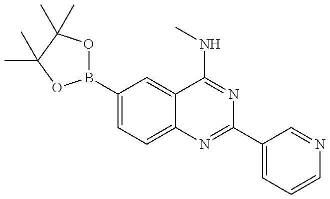 [SMILES: CNc1nc(-c2cccnc2)nc2ccc(B3OC(C)(C)C(C)(C)O3)cc12] | 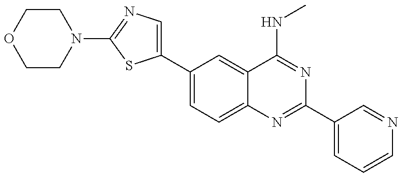 [SMILES: CNc1nc(-c2cccnc2)nc2ccc(-c3cnc(N4CCOCC4)s3)cc12] | 2 HCl | 1H NMR (300 MHz, DMSO) δ 10.22 (s, 1H), 9.62 (s, 1H), 9.04 (d, J = 7.7 Hz, 1H), 8.93 (d, J = 5.1 Hz, 1H), 8.58 (s, 1H), 8.22-8.07 (m, 2H), 7.95 (s, 1H), 7.88 (d, J = 3.8 Hz, 1H), 3.75 (s, 4H), 3.48 (s, 4H), 3.26 (d, J = 4.2 Hz, 3H). |
| 466 | 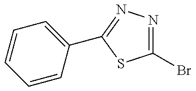 [SMILES: Brc1nnc(-c2ccccc2)s1] | 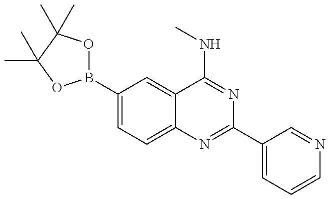 [SMILES: CNc1nc(-c2cccnc2)nc2ccc(B3OC(C)(C)C(C)(C)O3)cc12] | 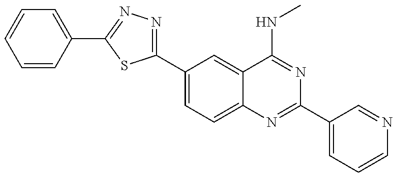 [SMILES: CNc1nc(-c2cccnc2)nc2ccc(-c3nnc(-c4ccccc4)s3)cc12] | 2 HCl | 1H NMR (300 MHz, DMSO) δ 9.63 (d, J = 1.6 Hz, 1H), 9.12-8.98 (m, 2H), 8.90 (dd, J = 5.1, 1.4 Hz, 1H), 8.51 (dd, J = 8.6, 1.5 Hz, 1H), 8.07 (d, J = 9.1 Hz, 1H), 8.05-7.98 (m, 2H), 7.87 (dd, J = 8.1, 5.2 Hz, 1H), 7.66-7.55 (m, 3H), 3.25 (d, J = 4.2 Hz, 3H). |
| 467 | 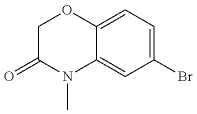 | 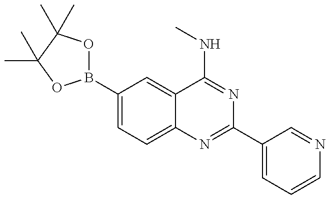 [SMILES: CNc1nc(-c2cccnc2)nc2ccc(B3OC(C)(C)C(C)(C)O3)cc12] | 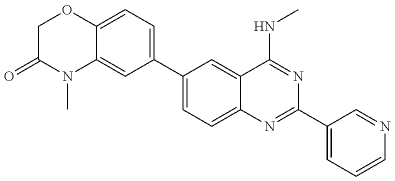 [SMILES: CNc1nc(-c2cccnc2)nc2ccc(-c3ccc4c(c3)N(C)C(=O)CO4)cc12] | 2 HCl | 1H NMR (300 MHz, DMSO) δ 10.64 (s, 1H), 9.62 (d, J = 1.6 Hz, 1H), 9.00 (s, 2H), 8.93 (d, J = 5.0 Hz, 1H), 8.38 (d, J = 8.5 Hz, 1H), 8.19 (d, J = 8.5 Hz, 1H), 7.84 (dd, J = 7.6, 5.7 Hz, 1H), 7.65 (s, 1H), 7.53 (d, J = 8.3 Hz, 1H), 7.14 (d, J = 8.3 Hz, 1H), 4.71 (s, 2H), 3.45 (s, 3H), 3.29 (d, J = 4.1 Hz, 3H). |
| 468 | 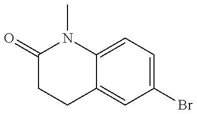 | 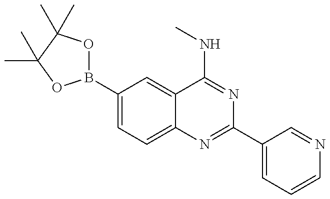 [SMILES: CNc1nc(-c2cccnc2)nc2ccc(B3OC(C)(C)C(C)(C)O3)cc12] | 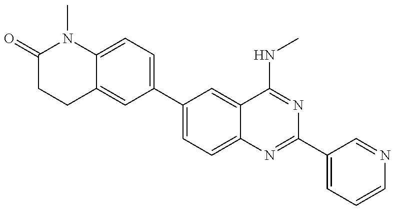 [SMILES: CNc1nc(-c2cccnc2)nc2ccc(-c3ccc4c(c3)CCC(=O)N4C)cc12] | 2 HCl | 1H NMR (300 MHz, DMSO) δ 10.33 (s, 1H), 9.62 (d, J = 0.8 Hz, 1H), 9.00 (d, J = 5.9 Hz, 1H), 8.93 (d, J = 4.8 Hz, 1H), 8.87 (s, 1H), 8.36 (d, J = 8.5 Hz, 1H), 8.19 (d, J = 8.6 Hz, 1H), 7.93-7.75 (m, 3H), 7.24 (d, J = 8.9 Hz, 1H), 3.37-3.24 (m, 6H), 2.97 (t, J = 7.3 Hz, 2H), 2.67- 2.56 (m, 2H). |
| 469 | 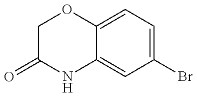 [SMILES: O=C1COc2ccc(Br)cc2N1] | 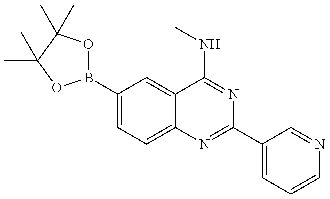 [SMILES: CNc1nc(-c2cccnc2)nc2ccc(B3OC(C)(C)C(C)(C)O3)cc12] | 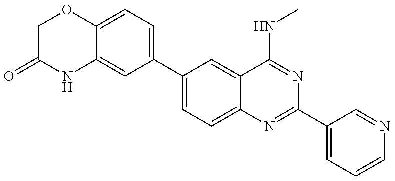 [SMILES: CNc1nc(-c2cccnc2)nc2ccc(-c3ccc4c(c3)NC(=O)CO4)cc12] | 2 HCl | 1H NMR (300 MHz, DMSO) δ 10.93 (s, 1H), 9.60 (d, J = 1.9 Hz, 1H), 9.02-8.88 (m, 2H), 8.69 (s, 1H), 8.22-8.08 (m, 2H), 7.90-7.78 (m, 1H), 7.41 (dd, J = 8.4, 2.2 Hz, 1H), 7.26 (d, J = 2.1 Hz, 1H), 7.14 (d, J = 8.5 Hz, 1H), 4.65 (s, 2H), 3.29 (d, J = 4.3 Hz, 3H). |
| 470 | 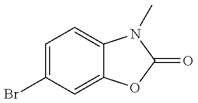 | 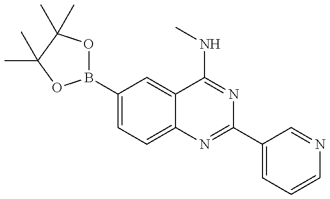 [SMILES: CNc1nc(-c2cccnc2)nc2ccc(B3OC(C)(C)C(C)(C)O3)cc12] | 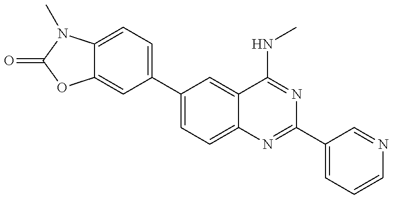 [SMILES: CNc1nc(-c2cccnc2)nc2ccc(-c3ccc4c(c3)oc(=O)n4C)cc12] | 2 HCl | 1H NMR (300 MHz, DMSO) δ 10.17 (s, 1H), 9.61 (d, J = 2.0 Hz, 1H), 8.98 (d, J = 8.7 Hz, 1H), 8.92 (d, J = 4.5 Hz, 1H), 8.84 (s, 1H), 8.36 (d, J = 8.7 Hz, 1H), 8.14 (d, J = 7.7 Hz, 1H), 7.90 (s, 1H), 7.88-7.75 (m, 2H), 7.42 (d, J = 8.1 Hz, 1H), 3.38 (s, 3H), 3.30 (d, J = 3.8 Hz, 3H). |
| 471 | 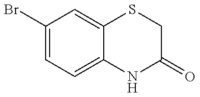 [SMILES: O=C1CSc2cc(Br)ccc2N1] | 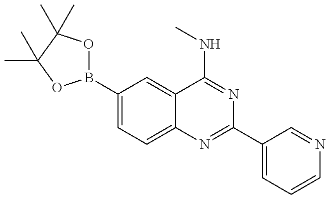 [SMILES: CNc1nc(-c2cccnc2)nc2ccc(B3OC(C)(C)C(C)(C)O3)cc12] | 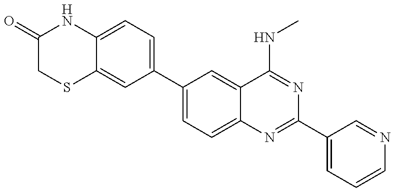 [SMILES: CNc1nc(-c2cccnc2)nc2ccc(-c3ccc4c(c3)SCC(=O)N4)cc12] | 2 HCl | 1H NMR (300 MHz, DMSO) δ 10.76 (s, 1H), 9.62 (s, 1H), 9.00 (d, J = 7.2 Hz, 1H), 8.93 (d, J = 4.2 Hz, 1H), 8.82 (s, 1H), 8.33 (d, J = 8.8 Hz, 1H), 8.16 (d, J = 8.9 Hz, 1H), 7.93- 7.79 (m, 2H), 7.73 (d, J = 8.6 Hz, 1H), 7.11 (d, J = 8.2 Hz, 1H), 3.55 (s, 2H), 3.29 (d, J = 3.9 Hz, 3H). |
| 472 | 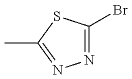 | 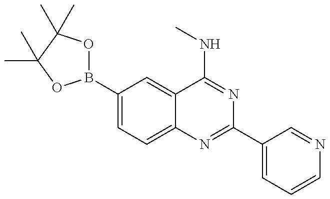 [SMILES: CNc1nc(-c2cccnc2)nc2ccc(B3OC(C)(C)C(C)(C)O3)cc12] | 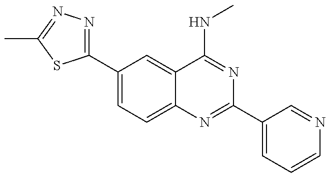 [SMILES: CNc1nc(-c2cccnc2)nc2ccc(-c3nnc(C)s3)cc12] | 2 HCl | 1H NMR (300 MHz, DMSO) δ 10.06 (s, 1H), 9.64 (d, J = 1.8 Hz, 1H), 9.12 (d, J = 7.5 Hz, 1H), 9.02 (s, 1H), 8.96 (d, J = 5.2 Hz, 1H), 8.48 (d, J = 8.7 Hz, 1H), 8.14 (d, J = 8.4 Hz, 1H), 7.94 (dd, J = 7.4, 6.0 Hz, 1H), 3.25 (d, J = 4.2 Hz, 3H), 2.82 (s, 3H). |
| 473 | 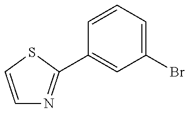 [SMILES: Brc1cccc(-c2nccs2)c1] | 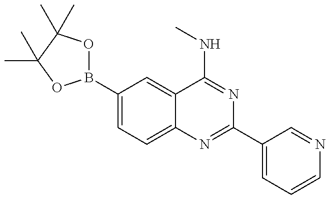 [SMILES: CNc1nc(-c2cccnc2)nc2ccc(B3OC(C)(C)C(C)(C)O3)cc12] | 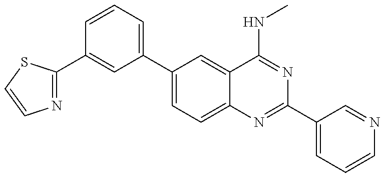 [SMILES: CNc1nc(-c2cccnc2)nc2ccc(-c3cccc(-c4nccs4)c3)cc12] | | 1H NMR (300 MHz, DMSO) δ 9.64 (d, J = 2.1 Hz, 1H), 8.78 (d, J = 8.0 Hz, 1H), 8.71 (d, J = 4.6 Hz, 1H), 8.70-8.64 (m, 2H), 8.37 (s, 1H), 8.20 (d, J = 8.7 Hz, 1H), 7.99 (s, 1H), 7.98-7.94 (m, 1H), 7.95-7.83 (m, 3H), 7.67 (t, J = 7.7 Hz, 1H), 7.54 (dd, J = 7.9, 4.8 Hz, 1H), 3.19 (d, J = 4.2 Hz, 3H). |
| 474 | 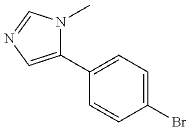 | 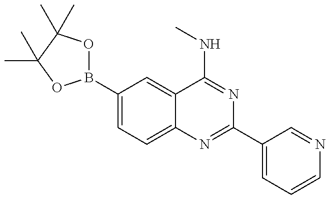 [SMILES: CNc1nc(-c2cccnc2)nc2ccc(B3OC(C)(C)C(C)(C)O3)cc12] | 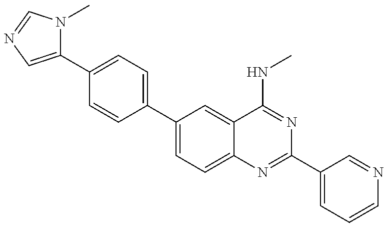 [SMILES: CNc1nc(-c2cccnc2)nc2ccc(-c3ccc(-c4cncn4C)cc3)cc12] | | 1H NMR (300 MHz, DMSO) δ 9.64 (d, J = 2.1 Hz, 1H), 8.77 (dd, J = 6.1, 1.9 Hz, 1H), 8.71-8.64 (m, 3H), 8.20 (d, J = 8.7 Hz, 1H), 7.97 (d, J = 8.5 Hz, 2H), 7.91-7.82 (m, 3H), 7.54 (dd, J = 7.7, 5.1 Hz, 1H), 7.29 (s, J = 1.1 Hz, 1H), 7.01 (s, J = 1.1 Hz, 1H), 3.81 (s, 3H), 3.18 (d, J = 4.2 Hz, 3H). |
| 475 | 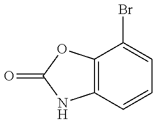 [SMILES: O=c1[nH]c2cccc(Br)c2o1] | 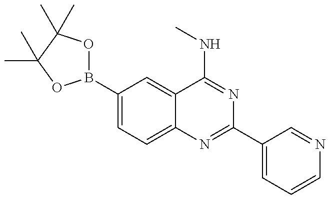 [SMILES: CNc1nc(-c2cccnc2)nc2ccc(B3OC(C)(C)C(C)(C)O3)cc12] | 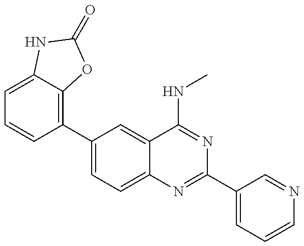 [SMILES: CNc1nc(-c2cccnc2)nc2ccc(-c3cccc4[nH]c(=O)oc34)cc12] | | 1H NMR (300 MHz, DMSO) δ 11.87 (s, 1H), 9.65 (s, 1H), 8.78 (d, J = 8.1 Hz, 1H), 8.68 (d, J = 4.7 Hz, 1H), 8.53 (d, J = 3.8 Hz, 1H), 8.41 (s, 1H), 7.96 (d, J = 8.9 Hz, 1H), 7.86 (d, J = 8.7 Hz, 1H), 7.54 (dd, J = 7.8, 4.5 Hz, 1H), 7.35 (d, J = 7.6 Hz, 2H), 7.28-7.17 (m, 1H), 3.17 (d, J = 4.1 Hz, 3H). |
| 476 | 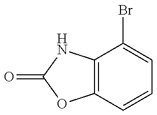 [SMILES: O=c1[nH]c2c(Br)cccc2o1] | 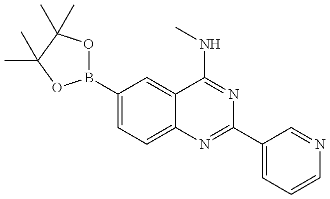 [SMILES: CNc1nc(-c2cccnc2)nc2ccc(B3OC(C)(C)C(C)(C)O3)cc12] | 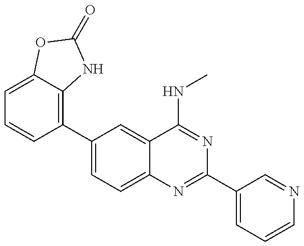 [SMILES: CNc1nc(-c2cccnc2)nc2ccc(-c3cccc4oc(=O)[nH]c34)cc12] | | 1H NMR (300 MHz, DMSO) δ 11.82 (s, 1H), 9.64 (s, 1H), 8.78 (d, J = 6.8 Hz, 1H), 8.67 (s, 2H), 8.60 (s, 1H), 8.18 (d, J = 8.6 Hz, 1H), 7.90 (d, J = 8.4 Hz, 1H), 7.64-7.40 (m, 3H), 7.30 (t, J = 7.7 Hz, 1H), 7.13 (d, J = 7.2 Hz, 1H), 3.17 (d, J = 1.2 Hz, 3H). |
| 477 | 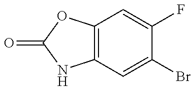 [SMILES: O=c1[nH]c2cc(Br)c(F)cc2o1] | 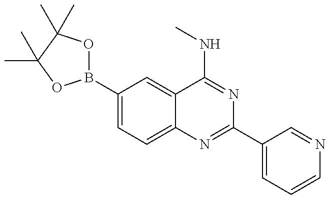 [SMILES: CNc1nc(-c2cccnc2)nc2ccc(B3OC(C)(C)C(C)(C)O3)cc12] | 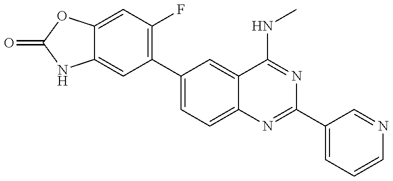 [SMILES: CNc1nc(-c2cccnc2)nc2ccc(-c3cc4[nH]c(=O)oc4cc3F)cc12] | | 1H NMR (300 MHz, DMSO) δ 11.94 (s, 1H), 9.63 (s, 1H), 8.77 (d, J = 7.9 Hz, 1H), 8.74-8.64 (m, 2H), 8.61 (s, 1H), 8.40 (s, 1H), 7.91 (s, 1H), 7.84 (d, J = 8.6 Hz, 1H), 7.61-7.47 (m, 2H), 7.26 (d, J = 6.8 Hz, 1H), 3.15 (d, J = 4.2 Hz, 3H). |
| 478 | 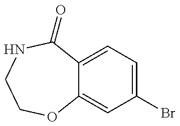 [SMILES: O=C1NCCOc2cc(Br)ccc21] | 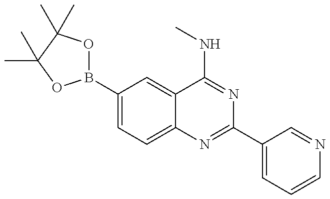 [SMILES: CNc1nc(-c2cccnc2)nc2ccc(B3OC(C)(C)C(C)(C)O3)cc12] | 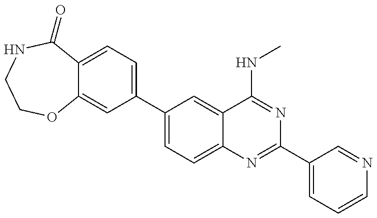 [SMILES: CNc1nc(-c2cccnc2)nc2ccc(-c3ccc4c(c3)OCCNC4=O)cc12] | | 1H NMR (300 MHz, DMSO) δ 9.63 (d, J = 1.2 Hz, 1H), 8.77 (d, J = 8.2 Hz, 1H), 8.74-8.63 (m, 3H), 8.40 (s, 1H), 8.19 (d, J = 8.9 Hz, 1H), 7.93 (d, J = 8.3 Hz, 1H), 7.84 (d, J = 8.7 Hz, 1H), 7.63 (dd, J = 8.1, 1.6 Hz, 1H), 7.57-7.50 (m, 2H), 4.37 (t, J = 4.6 Hz, 2H), 3.44- 3.35 (m, 2H), 3.18 (d, J = 4.2 Hz, 3H). |
| 479 | 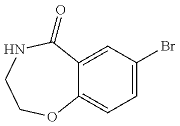 [SMILES: O=C1NCCOc2ccc(Br)cc21] | 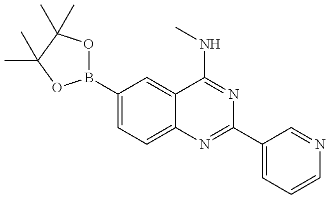 [SMILES: CNc1nc(-c2cccnc2)nc2ccc(B3OC(C)(C)C(C)(C)O3)cc12] | 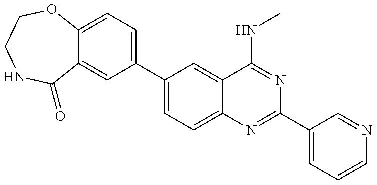 [SMILES: CNc1nc(-c2cccnc2)nc2ccc(-c3ccc4c(c3)C(=O)NCCO4)cc12] | | 1H NMR (300 MHz, DMSO) δ 9.63 (d, J = 1.2 Hz, 1H), 8.76 (d, J = 8.0 Hz, 1H), 8.69 (dd, J = 11.1, 5.0 Hz, 2H), 8.57 (s, 1H), 8.49 (t, J = 5.5 Hz, 1H), 8.23 (d, J = 2.3 Hz, 1H), 8.10 (d, J = 8.7 Hz, 1H), 7.93 (dd, J = 8.5, 2.4 Hz, 1H), 7.83 (d, J = 8.7 Hz, 1H), 7.53 (dd, J = 7.9, 4.8 Hz, 1H), 7.17 (d, J = 8.5 Hz, 1H), 4.34 (t, J = 4.5 Hz, 2H), 3.37 (d, J = 4.6 Hz, 2H), 3.17 (d, J = 4.1 Hz, 3H). |
| 480 | 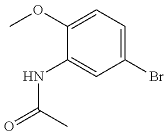 | 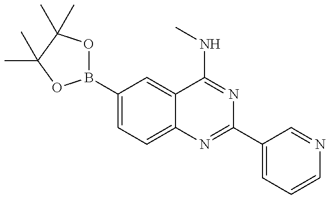 [SMILES: CNc1nc(-c2cccnc2)nc2ccc(B3OC(C)(C)C(C)(C)O3)cc12] | 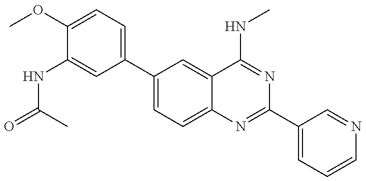 [SMILES: CNc1nc(-c2cccnc2)nc2ccc(-c3ccc(OC)c(NC(C)=O)c3)cc12] | | 1H NMR (300 MHz, DMSO) δ 9.62 (d, J = 1.2 Hz, 1H), 9.31 (s, 1H), 8.76 (dd, J = 9.8, 1.8 Hz, 1H), 8.66 (dd, J = 4.7, 1.7 Hz, 1H), 8.61 (d, J = 4.6 Hz, 1H), 8.46 (s, 1H), 8.34 (s, 1H), 8.00 (d, J = 8.8 Hz, 1H), 7.83 (d, J = 8.7 Hz, 1H), 7.60-7.47 (m, 2H), 7.18 (d, J = 8.6 Hz, 1H), 3.89 (s, 3H), 3.16 (d, J = 4.3 Hz, 4H), 2.11 (s, 3H). |
| 481 | | 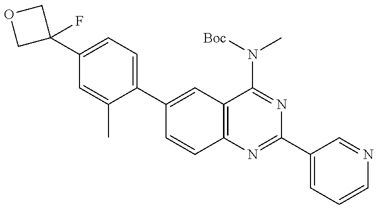 [SMILES: Cc1cc(C2(F)COC2)ccc1-c1ccc2nc(-c3cccnc3)nc(N(C)C(=O)OC(C)(C)C)c2c1] | 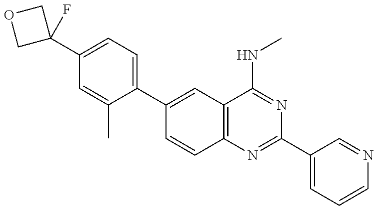 [SMILES: CNc1nc(-c2cccnc2)nc2ccc(-c3ccc(C4(F)COC4)cc3C)cc12] | | 1H NMR (400 MHz, DMSO) δ 9.65 (dd, J = 2.1, 0.8 Hz, 1H), 8.83- 8.74 (m, 1H), 8.69 (dd, J = 4.8, 1.7 Hz, 1H), 8.55-8.45 (m, 1H), 8.25 (d, J = 1.6 Hz, 1H), 7.86 (d, J = 8.5 Hz, 1H), 7.81 (dd, J = 8.5, 1.8 Hz, 1H), 7.62-7.53 (m, 2H), 7.51 (d, J = 8.0 Hz, 1H), 7.44 (d, J = 7.9 Hz, 1H), 5.09-4.92 (m, 4H), 3.16 (d, J = 4.5 Hz, 3H), 2.36 (s, 3H). |
| 482 | | 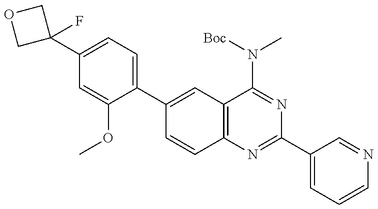 [SMILES: COc1cc(C2(F)COC2)ccc1-c1ccc2nc(-c3cccnc3)nc(N(C)C(=O)OC(C)(C)C)c2c1] | 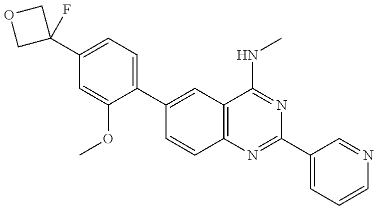 [SMILES: CNc1nc(-c2cccnc2)nc2ccc(-c3ccc(C4(F)COC4)cc3OC)cc12] | | 1H NMR (400 MHz, DMSO) δ 9.65 (dd, J = 2.1, 0.8 Hz, 1H), 8.86- 8.72 (m, 1H), 8.69 (dd, J = 4.7, 1.7 Hz, 1H), 8.55-8.44 (m, 1H), 8.34 (d, J = 1.7 Hz, 1H), 7.95 (dd, J = 8.6, 1.9 Hz, 1H), 7.81 (d, J = 8.6 Hz, 1H), 7.64-7.46 (m, 2H), 7.40- 7.20 (m, 2H), 5.12-4.93 (m, 4H), 3.88 (s, 3H), 3.16 (d, J = 4.5 Hz, 3H). |

| | ¹H NMR | Purity | Method | | Retention | LCMS |
|---|---|---|---|---|---|---|
| Number | Solvent | percent | of Coupling | LCMS | Time | Method |
| 449 | DMSO | 96 | Method | 406.0 | t = 1.796 | Method B |
| | | | R5 | (M + 1) | min | (NH4HCO3) |
| 450 | DMSO | 95 | Method | 368.1 | t = 1.435 | Method B |
| | | | R1 | (M + 1) | min | (NH4HCO3 |
| 451 | DMSO | >98 | Method | | | |
| | | | R5 | | | |
| 452 | DMSO | >98 | Method | | | |
| | | | R5 | | | |
| 453 | DMSO | >98 | Method | | | |
| | | | R4 | | | |
| 454 | DMSO | >98 | Method | | | |
| | | | R4 | | | |
| 455 | DMSO | >98 | Method | | | |
| | | | R4 | | | |
| 456 | DMSO | >98 | Method | | | |
| | | | R4 | | | |
| 457 | DMSO | >98 | Method | | | |
| | | | R4 | | | |
| 458 | DMSO | >98 | Method | | | |
| | | | R4 | | | |
| 459 | DMSO | 99 | Method | 382.2 | 1.61 | C |
| | | | R7 | (M + 1) | | |
| 460 | DMSO | 99 | Method | 379.2 | 1.75 | C |
| | | | R7 | (M + 1) | | |
| 461 | DMSO | 95 | Method | 371.2 | 1.89 | C |
| | | | R7 | (M + 1) | | |
| 462 | DMSO | 97 | Method | 370.1 | 1.74 | C |
| | | | R7 | (M + 1) | | |
| 463 | DMSO | 98 | Method | 370.1 | 1.73 | C |
| | | | R7 | (M + 1) | | |
| 464 | DMSO | 99 | Method | 396.4 | 2.42 | C |
| | | | R7 | (M + 1) | | |
| 465 | DMSO | 91 | Method | 405.1 | 1.89 | C |
| | | | R7 | (M + 1) | | |
| 466 | DMSO | 99 | Method | 397.2 | 2.28 | C |
| | | | R7 | (M + 1) | | |
| 467 | DMSO | 99 | Method | 398.2 | 1.95 | C |
| | | | R7 | (M + 1) | | |
| 468 | DMSO | 99 | Method | 396.3 | 1.91 | C |
| | | | R7 | (M + 1) | | |
| 469 | DMSO | 99 | Method | 384.2 | 1.71 | C |
| | | | R7 | (M + 1) | | |
| 470 | DMSO | 96 | Method | 384.2 | 2.03 | C |
| | | | R7 | (M + 1) | | |
| 471 | DMSO | 99 | Method | 400.1 | 1.79 | C |
| | | | R7 | (M + 1) | | |
| 472 | DMSO | 99 | Method | 335.0 | 1.68 | C |
| | | | R7 | (M + 1) | | |
| 473 | DMSO | 99 | Method | 396.0 | 2.22 | C |
| | | | R7 | (M + 1) | | |
| 474 | DMSO | 99 | Method | 393.2 | 1.72 | C |
| | | | R7 | (M + 1) | | |
| 475 | DMSO | 96 | Method | 370.2 | 1.83 | C |
| | | | R7 | (M + 1) | | |
| 476 | DMSO | 90 | Method | 370.2 | 1.76 | C |
| | | | R7 | (M + 1) | | |
| 477 | DMSO | 90 | Method | 388.1 | 1.80 | C |
| | | | R7 | (M + 1) | | |
| 478 | DMSO | 95 | Method | 398.2 | 1.64 | C |
| | | | R7 | (M + 1) | | |
| 479 | DMSO | 99 | Method | 398.1 | 1.67 | C |
| | | | R7 | (M + 1) | | |
| 480 | DMSO | 95 | Method | 400.3 | 1.83 | C |
| | | | R2 | (M + 1) | | |
| 481 | DMSO | >98 | | | | |
| 482 | DMSO | >98 | | | | |

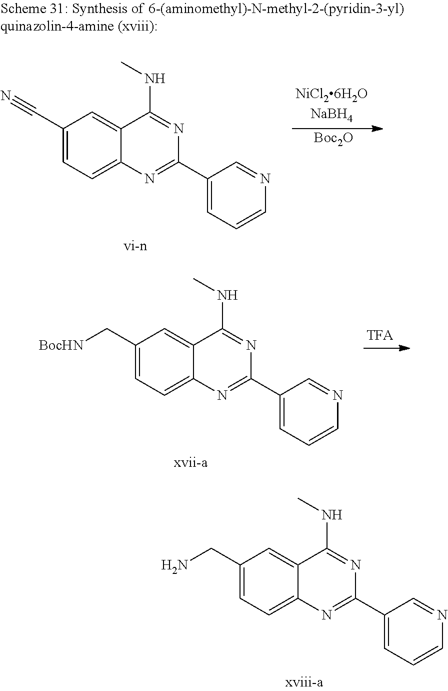 [SMILES: "Boc2O" ; CNc1nc(-c2cccnc2)nc2ccc(C#N)cc12 ; CNc1nc(-c2cccnc2)nc2ccc(CNC(=O)OC(C)(C)C)cc12 ; CNc1nc(-c2cccnc2)nc2ccc(CN)cc12]

### Method W: tert-Butyl (4-(methylamino)-2-(pyridin-3-yl)quinazolin-6-yl)methylcarbamate (xvii-a)

To a stirred solution of 4-(methylamino)-2-(pyridin-3-yl)quinazoline-6-carbonitrile (500 mg, 1.9 mmol) in dry methanol (15 mL) were added Boc₂O (830 mg, 3.8 mmol) and NiCl₂.6H₂O (690 mg, 2.9 mmol) at 0° C. NaBH₄ (1.80 g, 48.5 mmol) was added in small portions over 30 min. The reaction was exothermic and effervescent. The resulting reaction mixture containing a finely divided black precipitate was allowed to warm to room temperature and stirred for 4 h. After cooling and evaporation, the residue was purified by column chromatography (silica gel, EA:PE=10:1). The desired product was obtained as a white solid (250 mg) in 36% yield. MS m/z=366.0 (M+1), (Method B) (retention time=1.613 min)

### Method X: 6-(aminomethyl)-N-methyl-2-(pyridin-3-yl)quinazolin-4-amine (xviii-a)

To a stirred solution of tert-butyl (4-(methylamino)-2-(pyridin-3-yl)quinazolin-6-yl)methylcarbamate (250 mg, 0.68 mmol) in dry methanol (40 mL) was added TFA (20 mL). The reaction was heated to 55° C. for 48 h. After cooling and evaporation, the residue was purified on prep—HPLC (Condition C). The desired product was obtained as a white solid (120 mg) in a 67% yield. MS m/z=266.0 (M+1), (Method B) (retention time=1.297 min) ¹H-NMR (400 MHz, DMSO-d₆): δ 9.62 (s, 1H), 8.84-8.58 (m, 2H), 8.13 (s, 2H), 7.75 (s, 2H), 7.50 (s, 1H), 3.91 (s, 2H), 3.18 (s, 5H).

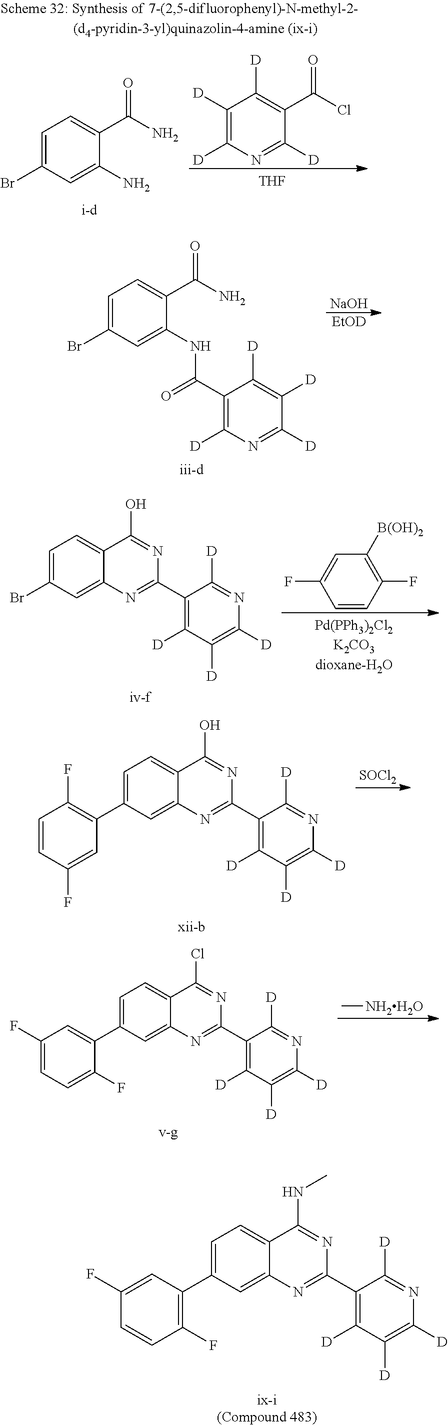

### N-(5-bromo-2-carbamoylphenyl)d₄-nicotinamide (iii-d)

To a solution of 2-amino-4-bromobenzamide (200 mg, 0.93 mmol, 1.0 eq.) in THF (10 mL) was added d₄-nicotinoyl chloride (270 mg, 1.86 mmol, 2.0 eq.) in anhydrous THF (5 mL) dropwise. The resulting mixture was stirred at room temperature overnight. After the reaction was completed, the resultant precipitate was filtered and dried in vacuo to give 240 mg of crude iii-d as a yellow solid (80% yield). LCMS m/z=324.0 (M+1) (Method B) (retention time=1.46 min)

### 7-Bromo-2-(d₄-pyridin-3-yl)quinazolin-4-ol (iv-f)

A mixture of N-(5-bromo-2-carbamoylphenyl)d₄-nicotinamide (240 mg, crude, 0.74 mmol, 1.0 eq) in EtOD (10 mL) was treated with NaOH (148 mg, 3.7 mmol, 5.0 eq). The resulting mixture was stirred at room temperature overnight. After the reaction was completed, the volatiles were removed in vacuo. Water (10 mL) was added to the residue and the mixture was adjusted to pH ˜1 or 2 by slow addition of aqueous HCl. The resultant precipitate was collected and dried to give 180 mg of 7-bromo-2-(d₄-pyridin-3-yl)quinazolin-4-ol as a yellow solid (81% yield after two steps). LCMS m/z=307.9, 308.9 (M+1) (Method B) (retention time=1.41 min).

### 7-(2,5-Difluorophenyl)-2-(d₄-pyridin-3-yl)quinazolin-4-ol (xii-b)

To a mixture of 7-bromo-2-(d₄-pyridin-3-yl)quinazolin-4-ol (180 mg, 0.59 mmol, 1.0 eq), 2,5-difluorophenylboronic acid (140 mg, 0.89 mmol, 1.5 eq), K₂CO₃ (244 mg, 1.77 mmol, 3.0 eq.) in dioxane (10 mL) and H₂O (1 mL) was added Pd(PPh₃)₂Cl₂ (38 mg, 0.047 mmol, 0.08 eq) under N₂ atmosphere. The resulting mixture was stirred at 100° C. under N₂ atmosphere overnight. After the reaction was completed, the mixture was filtered, and the filtrate was concentrated in vacuo. The residue was purified by reverse phase HPLC column to afford 160 mg of 7-(2,5-difluorophenyl)-2-(d₄-pyridin-3-yl)quinazolin-4-ol as a white solid (yield 80%). LCMS m/z=340.1, 341.1 (M+1) (Method B (retention time=1.56 min)

### 4-Chloro-7-(2,5-difluorophenyl)-2-(d₄-pyridin-3-yl)quinazoline (v-g)

7-(2,5-difluorophenyl)-2-(d₄-pyridin-3-yl)quinazolin-4-ol (160 mg, 0.47 mmol) was added to SOCl₂ (10 mL). The resulting mixture was stirred at 65° C. for 2 h. After the reaction was completed, the mixture was carefully poured into an ice-water solution. The pH was adjusted to 7 by slow addition of NH₄OH at 0° C. The resultant solid was collected to give 160 mg of 4-chloro-7-(2,5-difluorophenyl)-2-(d₄-pyridin-3-yl)quinazoline as a beige solid (quantitative yield). LCMS m/z=354.0 (M+1) (Method B) (retention time=2.07 min)

### 7-(2,5-Difluorophenyl)-N-methyl-2-(d₄-pyridin-3-yl)quinazolin-4-amine (ix-i, compound 483)

To a suspension of 4-chloro-7-(2,5-difluorophenyl)-2-(d₄-pyridin-3-yl)quinazoline (160 g, 0.45 mol) in THF (10 mL) was added a solution of methylamine (40 wt. % in H₂O, 5 mL) dropwise with cooling. The suspension was stirred at 60° C. for 3 h. After cooling, the precipitate was collected and dried to give the title compound. (130 mg, 82%). LCMS m/z=353.1 (M+1) (Method B) (retention time=1.72 min) ¹H NMR (400 MHz, DMSO-d₆): δ 9.59 (s, 1H), 8.33 (d, J=8.4 Hz, 1H), 7.97 (s, 1H), 7.73 (d, J=8.8 Hz, 1H), 7.66-7.61 (m, 1H), 7.46-7.45 (m, 1H), 7.38-7.33 (m, 1H), 3.19 (s, 3H).

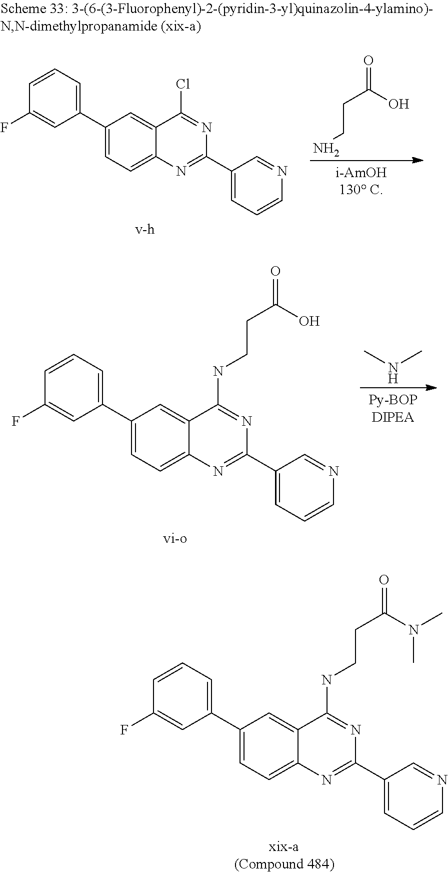

### 3-(6-(3-Fluorophenyl)-2-(pyridin-3-yl)quinazolin-4-ylamino)propanoic acid (vi-o)

To the solution of 4-chloro-6-(3-fluorophenyl)-2-(pyridin-3-yl)quinazoline (230 mg, 0.68 mmol, 1 eq) in 10 mL of isoamyl alcohol were added 3-aminopropanoic acid (121 mg, 1.36 mmol, 2.0 eq), DIPEA (263 mg, 2.04 mmol, 3.0 eq) and K₂CO₃ (94 mg, 0.68 mmol, 1.0 eq). The reaction mixture was heated to 130° C. overnight. After cooling, the volatiles were removed in vacuo and the residue was purified on reverse-phase-chromatography. Reverse-phase-chromatography condition C Retention time=3.6-4.1 min. The desired product was obtained as a yellow solid (90 mg) with a yield of 34.1%. LCMS m/z=389.0 (M+1) (Retention time=1.324 min) (Method B).

### 3-(6-(3-Fluorophenyl)-2-(pyridin-3-yl)quinazolin-4-ylamino)-N,N-dimethylpropanamide (xix-a) (Compound 484)

To the solution of 3-(6-(3-fluorophenyl)-2-(pyridin-3-yl)quinazolin-4-ylamino)propanoic acid (155 mg, 0.40 mmol, 1 eq) in 6 mL of DMF were added Py-BOP (There is only Py-Brop in the abbreviation section. Is Py-BOP correct?)(410 mg, 0.80 mmol, 2 eq) and DIPEA (155 mg, 1.20 mmol, 3 eq). The reaction mixture was stirred vigorously at room temperature for 2 h. Dimethylamine-hydrochloride (66 mg, 0.8 mmol, 2 eq) was added and the mixture was stirred at room temperature overnight. The resulting solution was partitioned between ethyl acetate and water. The combined organic layers were washed with brine and dried over Na₂SO₄. After filtration and concentration, the crude product was purified by reverse phase chromatography. Reverse-phase-chromatography condition C Retention time=5.6-6.8 min. The desired product was obtained as a white solid (19 mg) in an 11.4% yield. LCMS m/z=416.0 (M+1) (Retention time=1.695 min) (Method B). ¹H-NMR (400 MHz, DMSO-d₆): δ 9.62 (d, J=1.46 Hz, 1H), 8.76 (d, J=7.91 Hz, 1H), 8.72-8.62 (m, 3H), 8.19 (dd, J=8.72, 1.45 Hz, 1H), 7.87 (d, J=8.68 Hz, 1H), 7.74 (d, J=8.59 Hz, 2H), 7.64-7.50 (m, 2H), 7.33-7.21 (m, 1H), 3.93 (dd, J=12.66, 6.80 Hz, 2H), 2.97 (s, 3H), 2.88-2.79 (m, 5H).

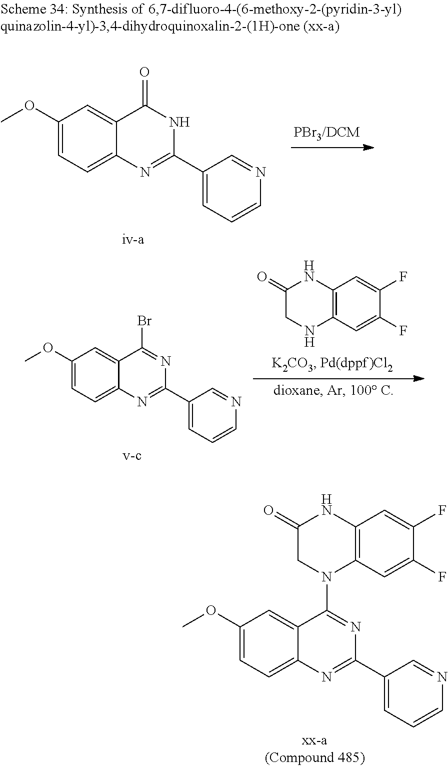 [SMILES: COc1ccc2nc(-c3cccnc3)[nH]c(=O)c2c1 ; COc1ccc2nc(-c3cccnc3)nc(Br)c2c1 ; O=C1CNc2cc(F)c(F)cc2N1 ; COc1ccc2nc(-c3cccnc3)nc(N3CC(=O)Nc4cc(F)c(F)cc43)c2c1]

### 4-Bromo-6-methoxy-2-(pyridin-3-yl)quinazoline (v-c)

To a suspension of 6-methoxy-2-(pyridin-3-yl)quinazolin-4(3H)-one (712 mg, 2.81 mmol) in dichloromethane (20 mL) was added PBr₃/dichloromethane (1.0 M, 10 mL) followed by DMF (0.25 mL). The mixture was stirred at 60° C. overnight. The volatiles were removed in vacuo and the residue was added to water (20 mL) Ammonia (5 mL) was added to neutralize the system until the pH was adjusted to 7-8. The precipitate was collected to give 4-bromo-6-methoxy-2-(pyridin-3-yl)quinazoline (570 mg, 64%). LCMS m/z=315.7 (M+1) (Method A) (retention time=1.64 min).

### 6,7-Difluoro-4-(6-methoxy-2-(pyridin-3-yl)quinazolin-4-yl)-3,4-dihydroquinoxalin-2(1H)-one (xx-a, compound 485)

A mixture of 4-bromo-6-methoxy-2-(pyridin-3-yl)quinazoline (100 mg, 0.31 mmol, 1.0 equiv), 6,7-difluoro-3,4-dihydroquinoxalin-2(1H)-one (58 mg, 0.31 mmol, 1.0 equiv), potassium carbonate (87 mg, 0.63 mmol, 2.0 eq) and Pd(dppf)Cl₂ (25 mg, 10 mol %) in dioxane (30 mL) was stirred at 100° C. under argon atmosphere overnight. The volatiles were removed in vacuo. The residue was purified by prepative HPLC to afford the desired product as a yellow solid (31 mg, 23%). LCMS m/z=420.0 (M+1) (Method A) (retention time=1.20 min) ¹H-NMR (400 MHz, CDCl₃): δ 10.95 (s, 1H), 9.67 (s, 1H), 8.97 (d, J=8.0 Hz, 1H), 8.80 (d, J=2.8 Hz, 1H), 8.02 (d, J=9.2 Hz, 1H), 7.76 (dd, J=8.0, 5.2 Hz, 1H), 7.61 (dd, J=9.0, 2.6 Hz, 1H), 7.10 (dd, J=11.2, 8.0 Hz, 1H), 6.99 (dd, J=11.6, 8.0 Hz, 1H), 6.76 (d, J=2.8 Hz, 1H), 4.71 (s, 2H), 3.61 (s, 3H).

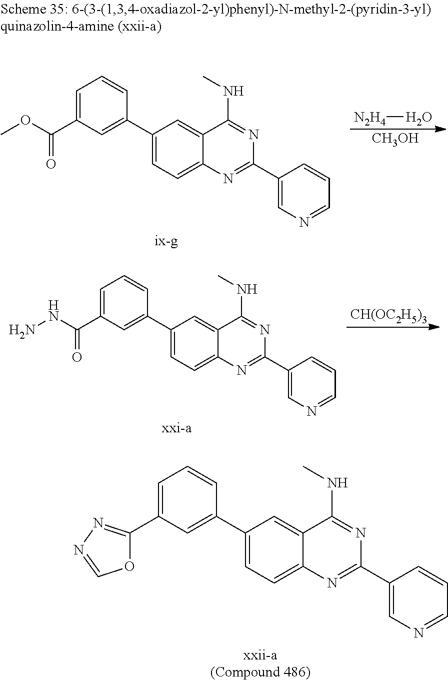

### 3-(4-(Methylamino)-2-(pyridin-3-yl)quinazolin-6-yl)benzohydrazide(xxi-a)

A mixture of methyl 3-(4-(methylamino)-2-(pyridin-3-yl)quinazolin-6-yl)benzoate (300 mg, 0.81 mmol) and N₂H₄—H₂O (4 ml) in methanol (20 mL) was heated to reflux overnight. After cooling, the reaction was concentrated down and the residue was washed with water (2×20 mL) and dried to give 155 mg of 3-(4-(methylamino)-2-(pyridin-3-yl)quinazolin-6-yl)benzohydrazide in a 74.5% yield. LCMS m/z=371 (M+1) (method B) (Retention time=1.40 min)

### 6-(3-(1,3,4-oxadiazol-2-yl)phenyl)-N-methyl-2-(pyridin-3-yl)quinazolin-4-amine (xxii-a, compound 486)

A solution of 3-(4-(methylamino)-2-(pyridin-3-yl)quinazolin-6-yl)benzohydrazide (105 mg, 0.28 mmol) in triethoxymethane (5 ml) was stirred at 140° C. overnight. After cooling and evaporation, the residue was purified by column chromatography (silica gel, ethyl acetate-petroleum ether, 2:1, and 1% TEA) to give the desired product 6-(3-(1,3,4-oxadiazol-2-yl)phenyl)-N-methyl-2-(pyridin-3-yl)quinazolin-4-amine (15.9 mg, 14.7%). LCMS m/z=381.1 (M+1) (method B) (Retention time=1.58 min) ¹H-NMR (400 MHz, DMSO): δ 9.66 (s, 1H), 9.45 (s, 1H), 8.80 (d, J=8.0 Hz, 1H), 8.74-8.69 (m, 3H), 8.49 (s, 1H), 8.25-8.22 (m, 1H), 8.11 (dd, J=17.6, 7.6 Hz, 2H), 7.91 (d, J=9.2 Hz, 1H), 7.79 (t, J=7.6 Hz, 1H), 7.56 (dd, J=7.6, 4.4 Hz, 1H), 3.21 (d, J=4.4 Hz, 3H).

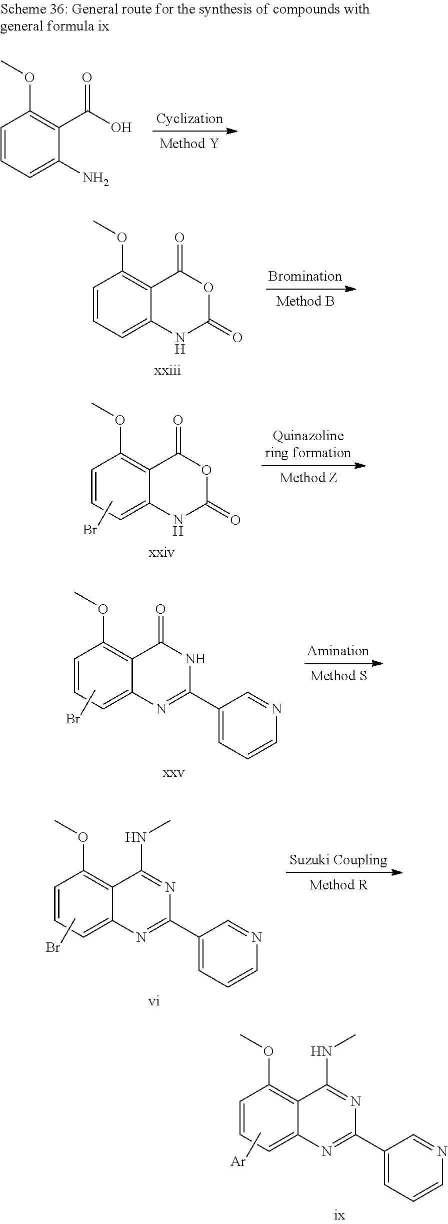

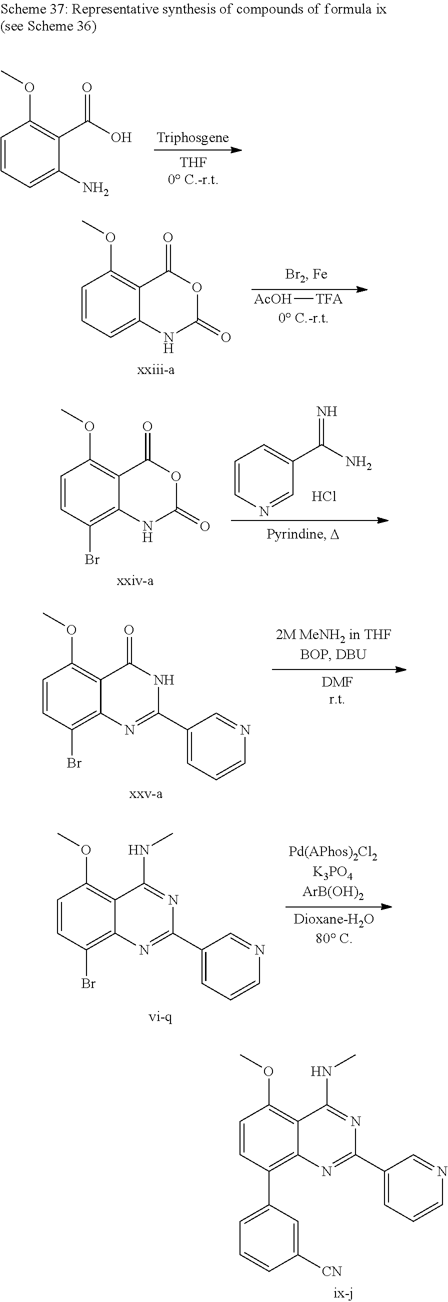

### 5-Methoxy-1H-benzo[d][1,3]oxazine-2,4-dione (xxiii-a)

In a 100 mL pear shaped flask was added 2-amino-6-methoxybenzoic acid (2.0 g, 11.96 mmol) in THF (25 ml) to give a yellow solution. Triphosgene (1.420 g, 4.79 mmol) was added slowly. The mixture was stirred overnight at room temperature. The reaction mixture was diluted with water (50 mL). The resultant precipitate was collected by filtration and dried to give 2.0 g of the desired product as a pale brown solid in an 87% yield. ¹H NMR (300 MHz, DMSO) δ 11.58 (s, 1H), 7.62 (t, J=8.3 Hz, 1H), 6.81 (d, J=8.5 Hz, 1H), 6.67 (d, J=8.1 Hz, 1H), 3.86 (s, 3H).

### 6-Bromo-5-methoxy-1H-benzo[d][1,3]oxazine-2,4-dione (xxiv-b)

In a 100 mL pear shaped flask was added 5-methoxy-1H-benzo[d][1,3]oxazine-2,4-dione (1.180 g, 6.11 mmol) in CH₂Cl₂ (10 ml) and DMF (5.00 ml) to give a yellow solution. N-Bromosuccinimide (1.522 g, 8.55 mmol) was added slowly at 0° C. The mixture was stirred overnight at room temperature. The reaction mixture was diluted with water (30 mL) and CH₂Cl₂ was evaporated in vacuo. The resultant precipitate was collected by filtration and dried. The precipitate was purified via ISCO (silica gel, 1:0 to 9:1 CH₂Cl₂/MeOH; 40 gm column) to give 0.72 g of the desired product as a light yellow solid in a 43% yield. ¹H NMR (300 MHz, DMSO) δ 11.79 (s, 1H), 7.93 (d, J=8.8 Hz, 1H), 6.86 (d, J=8.8 Hz, 1H), 3.80 (s, 3H).

### 8-bromo-5-methoxy-1H-benzo[d][1,3]oxazine-2,4-dione (xxiv-a)

In a 100 mL pear shaped flask were added 5-methoxy-1H-benzo[d][1,3]oxazine-2,4-dione (0.300 g, 1.553 mmol) and iron powder (5.20 mg, 0.093 mmol) in acetic acid (9 ml) and TFA (3 mL) to give a orange solution. Bromine (0.119 ml, 2.330 mmol) in TFA (3 mL) was added slowly at 0° C. The reaction mixture was stirred for 2 h at room temperature and then diluted with water (30 mL). The resultant precipitate was collected by filtration and dried to give 0.372 g of the 8-bromo product as a light brown solid in an 88% yield. ¹H NMR (300 MHz, DMSO) δ 10.70 (s, 1H), 7.90 (d, J=9.1 Hz, 1H), 6.84 (d, J=9.1 Hz, 1H), 3.88 (s, 3H).

### 8-bromo-5-methoxy-2-(pyridin-3-yl)quinazolin-4(3H)-one (xxv-a)

In a 100 mL pear shaped flask were added 8-bromo-5-methoxy-1H-benzo[d][1,3]oxazine-2,4-dione (2.65 g, 9.74 mmol) and 3-amidinopyridine hydrochloride (3.07 g, 19.48 mmol) in pyridine (15 ml) to give a yellow suspension. The mixture was heated at reflux for 2 h. After cooling to room temperature, the reaction mixture was diluted with water (50 mL). The resultant precipitate was collected by filtration and dried to give 2.36 g of the desired product as white solid in a 73% yield. ¹H NMR (300 MHz, DMSO) δ 12.67 (s, 1H), 9.35 (d, J=2.2 Hz, 1H), 8.89-8.69 (m, J=3.9 Hz, 1H), 8.54 (d, J=8.0 Hz, 1H), 8.04 (d, J=8.9 Hz, 1H), 7.59 (dd, J=8.0, 4.8 Hz, 1H), 7.00 (d, J=8.9 Hz, 1H), 3.89 (s, 3H).

### 8-bromo-5-methoxy-N-methyl-2-(pyridin-3-yl)quinazolin-4-amine (vi-q)

In a 200 mL pear shaped flask were added 8-bromo-5-methoxy-2-(pyridin-3-yl)quinazolin-4(3H)-one (2.30 g, 6.92 mmol), BOP (3.98 g, 9.00 mmol), and 1,8-diazabicyclo[5.4.0]undec-7-ene (DBU) (2.071 ml, 13.85 mmol) in DMF (25 ml) to give a orange suspension. Methylamine (2M in THF, 6.92 ml, 13.85 mmol) was added. The mixture was stirred overnight at room temperature. The reaction mixture was diluted with water (70 mL). The resultant precipitate was collected by filtration and dried to give 2.39 g of the desired product as a light brown solid in a quantitative yield. ¹H NMR (300 MHz, DMSO) δ 9.64 (d, J=2.1 Hz, 1H), 8.77 (d, J=7.9 Hz, 1H), 8.70 (d, J=4.7 Hz, 1H), 8.55 (d, J=4.4 Hz, 1H), 8.01 (d, J=8.6 Hz, 1H), 7.56 (dd, J=7.9, 4.8 Hz, 1H), 6.95 (d, J=8.7 Hz, 1H), 4.01 (s, 3H), 3.17 (d, J=4.5 Hz, 3H).

### Method R2: 3—(5-methoxy-4-(methylamino)-2-(pyridin-3-yl)quinazolin-8-yl)benzonitrile dihydrochloride (ix-j)

In a 25 mL reaction vial were added 8-bromo-5-methoxy-N-methyl-2-(pyridin-3-yl)quinazolin-4-amine (0.2 g, 0.579 mmol), 3-cyanophenylboronic acid (0.128 g, 0.869 mmol), bis(di-tert-butyl(4-dimethylaminophenyl)phosphine)dichloropalladium(II) (0.033 g, 0.046 mmol), and potassium phosphate tribasic monohydrate (0.4 g, 1.738 mmol) in dioxane (7 ml) and water (0.7 ml) to give a yellow suspension. The mixture was heated at 80° C. for 5 h under argon. After cooling to room temperature, the reaction mixture was diluted with water (10 mL) and extracted with AcOEt (2×10 mL). Combined organic layers were washed with brine (1×15 mL). The organic layer was dried MgSO₄, filtered and concentrated. The residue was purified via ISCO (silica gel, 1:0 to 9:1 CH2Cl2/MeOH; 12 gm Gold column). The obtained free base was converted to the HCl salt by treatment with 4 M HCl-dioxane. The HCl salt was washed with MeOH to give 0.14 g of the desired product as a pale brown powder in a 55% yield. LCMS m/z=368, (M+1) (Method D (retention time=1.97 min). ¹H NMR (300 MHz, DMSO) δ 9.44 (s, 1H), 8.74-8.43 (m, 3H), 8.13 (s, 1H), 8.05 (d, J=8.0 Hz, 1H), 7.93-7.78 (m, 2H), 7.68 (t, J=7.8 Hz, 1H), 7.49 (dd, J=7.8, 4.6 Hz, 1H), 7.10 (d, J=8.5 Hz, 1H), 4.06 (s, 3H), 3.17 (d, J=4.5 Hz, 3H).

The compounds in the following table were prepared in a manner analogous to that described in Scheme 37

TABLE 9

| | | | | | | | | Method | | Reten- | |
|---|---|---|---|---|---|---|---|---|---|---|---|
| | Starting | Starting | | Salt | | ¹H NMR | Purity | of | | tion | LCMS |
| Number | Material 1 | Material 2 | Product | type | ¹H NMR | Solvent | percent | Coupling | LCMS | Time | Method |
| 487 | 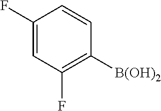 [SMILES: OB(O)c1ccc(F)cc1F] | 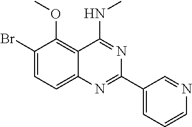 [SMILES: CNc1nc(-c2cccnc2)nc2ccc(Br)c(OC)c12] | 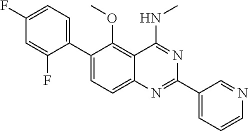 [SMILES: CNc1nc(-c2cccnc2)nc2ccc(-c3ccc(F)cc3F)c(OC)c12] | 2HCl | 1H-NMR (300 MHz, DMSO): δ 9.69 (s, 1H), 9.48 (s, 1H), 9.17 (d, J =8.1 Hz, 1H), 8.98 (d, J = 4.6 Hz, 1H), 8.07 (d, J = 8.5 Hz, 1H), 8.01- 7.80 (m, 2H), 7.74- 7.57 (m, 1H), 7.56- | DMSO | 99 | Method R2 | 379 (M + 1) | 1.60 | Method D |
| | | | | | 7.40 (m, 1H), 7.40- | | | | | | |
| | | | | | 7.22 (m, 1H), 3.50 | | | | | | |
| | | | | | (s, 3H), 3.31 (d, | | | | | | |
| | | | | | J = 4.4 Hz, 3H). | | | | | | |

| | | | | | | | | | | | |
|---|---|---|---|---|---|---|---|---|---|---|---|
| 488 | 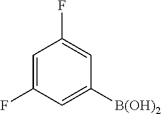 [SMILES: OB(O)c1cc(F)cc(F)c1] | 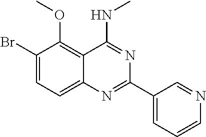 [SMILES: CNc1nc(-c2cccnc2)nc2ccc(Br)c(OC)c12] | 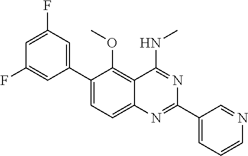 [SMILES: CNc1nc(-c2cccnc2)nc2ccc(-c3cc(F)cc(F)c3)c(OC)c12] | 2HCl | 1H NMR (300 MHz, DMSO) δ 9.64 (s, 1H), 9.34 (s, 1H), 9.07 (d, J = 6.4 Hz, 1H), 8.94 (d, J = 4.6 Hz, 1H), 8.10-7.80 (m, 3H), 7.54- 7.27 (m, 3H), 3.54 (s, 3H), 3.30 (d, J = 3.5 Hz, 3H). | DMSO | 99 | Method R2 | 379 (M + 1) | 1.67 | Method D |
| 489 | 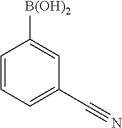 [SMILES: N#Cc1cccc(B(O)O)c1] | 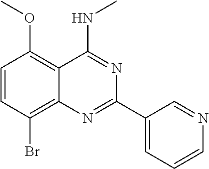 | 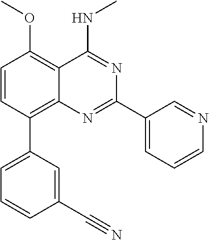 [SMILES: CNc1nc(-c2cccnc2)nc2c(-c3cccc(C#N)c3)ccc(OC)c12] | 2HCl | 1H NMR (300 MHz, DMSO) δ 9.44 (s, 1H), 8.74-8.43 (m, 3H), 8.13 (s, 1H), 8.05 (d, J = 8.0 Hz, 1H), 7.93-7.78 (m, 2H), 7.68 (t, J = 7.8 Hz, 1H), 7.49 (dd, J = 7.8, 4.6 Hz, 1H), 7.10 (d, J = 8.5 Hz, 1H), 4.06 (s, 3H), 3.17 (d, J = 4.5 Hz, 3H). | DMSO | 99 | Method R2 | 368 (M + 1) | 1.47 | Method D |
| 490 | 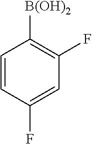 [SMILES: OB(O)c1ccc(F)cc1F] | 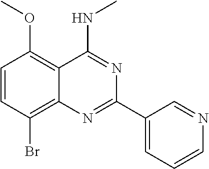 | 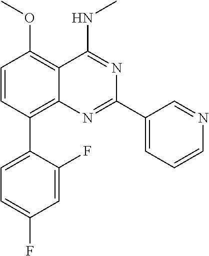 [SMILES: CNc1nc(-c2cccnc2)nc2c(-c3ccc(F)cc3F)ccc(OC)c12] | 2HCl | 1H NMR (300 MHz, DMSO) δ 9.37 (s, 1H), 8.96 (d, J = 7.7 Hz, 1H), 8.88 (d, J = 5.3 Hz, 1H), 8.67 (d, J = 4.3 Hz, 1H), 8.03- 7.88 (m, 1H), 7.74 (d, J = 8.3 Hz, 1H), 7.63- 7.47 (m, 1H), 7.42- 7.29 (m, 1H), 7.27-7.10 (m, 2H), 4.07 (s, 3H), 3.19 (d, J = 4.5 Hz, 3H). | DMSO | 99 | Method R2 | 378 (M + 1) | 2.39 | Method C |
| 491 | 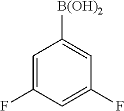 [SMILES: OB(O)c1cc(F)cc(F)c1] | 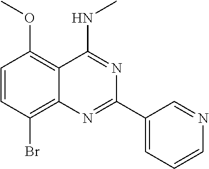 [SMILES: CNc1nc(-c2cccnc2)nc2c(Br)ccc(OC)c12] | 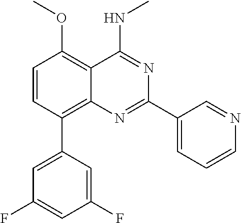 [SMILES: CNc1nc(-c2cccnc2)nc2c(-c3cc(F)cc(F)c3)ccc(OC)c12] | 2HCl | 1H NMR (300 MHz, DMSO) δ 9.47 (d, J = 1.2 Hz, 1H), 8.65 (dd, J = 4.7, 1.5 Hz, 1H), 8.60 (d, J = 8.0 Hz, 1H), 8.54 (d, J = 4.5 Hz, 1H), 7.83 (d, J = 8.5 Hz, 1H), 7.51 (dd, J = 7.9, 4.9 Hz, 1H), 7.45 (d, J = 7.1 Hz, 2H), 7.23 (t, J = 9.4 Hz, 1H), 7.09 (d, J = 8.5 Hz, 1H), | DMSO | 99 | Method R2 | 379 (M + 1) | 2.46 | Method C |
| | | | | | 1.06 (s, 3H), 3.17 (d, | | | | | | |
| | | | | | J = 4.6 Hz, 3H). | | | | | | |
| 492 | 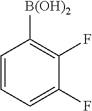 [SMILES: OB(O)c1cccc(F)c1F] | 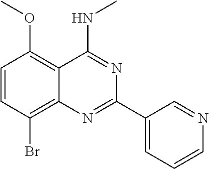 [SMILES: CNc1nc(-c2cccnc2)nc2c(Br)ccc(OC)c12] | 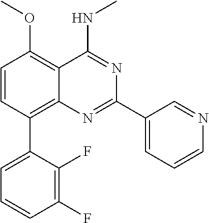 [SMILES: CNc1nc(-c2cccnc2)nc2c(-c3cccc(F)c3F)ccc(OC)c12] | 2HCl | 1H NMR (300 MHz, cd3od) δ 9.47 (s, 1H), 9.30 (d, J = 7.8 Hz, 1H), 8.88 (d, J = 5.6 Hz, 1H), 8.11 (dd, J = 8.1, 5.7 Hz, 1H), 7.78 (d, J = 8.4 Hz, 1H), 7.43- 7.26 (m, 3H), 7.22 (d, J = 8.4 Hz, 1H), 4.17 (s, 3H), 3.31 (s, 3H). | CD3OD | 99 | Method R2 | 379 (M + 1) | 2.31 | Method C |
| 493 | 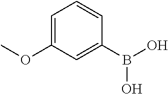 | 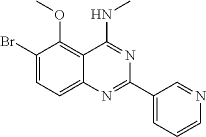 | 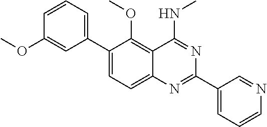 [SMILES: CNc1nc(-c2cccnc2)nc2ccc(-c3cccc(OC)c3)c(OC)c12] | 2HCl | 1H NMR (300 MHz, DMSO) δ 9.71 (d, J = 2.0 Hz, 1H), 9.64 (s, 1H), 9.18 (d, J = 8.3 Hz, 1H), 9.08- 8.90 (m, 1H), 8.12 (d, J = 8.6 Hz, 1H), 8.07-7.84 (m, 2H), 7.46 (t, J = 8.1 Hz, | DMSO | 99 | Method R2 | 373 (M + 1) | 1.53 | Method D |
| | | | | | 1H), 7.33-7.14 (m, | | | | | | |
| | | | | | 2H), 7.13-6.96 (m, | | | | | | |
| | | | | | 1H), 3.82 (s, 3H), | | | | | | |
| | | | | | 3.52 (s, 3H), 3.32 | | | | | | |
| | | | | | (d, J = 4.6 Hz, 3H). | | | | | | |
| 494 | 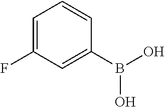 | 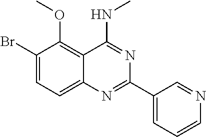 | 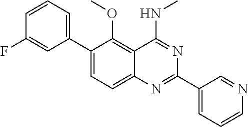 [SMILES: CNc1nc(-c2cccnc2)nc2ccc(-c3cccc(F)c3)c(OC)c12] | 2HCl | 1H NMR (300 MHz, DMSO) δ 9.65 (s, 1H), 9.50 (s, 1H), 9.10 (d, J = 6.1 Hz, 1H), 8.96 (d, J = 4.3 Hz, 1H), 8.11- 7.81 (m, 3H), 7.70- 7.43 (m, 3H), 7.41- 7.22 (m, 1H), 3.51 | DMSO | 99 | Method R2 | 361 (M + 1) | 1.64 | Method D |
| | | | | | (s, 3H), 3.32 (d, J = | | | | | | |
| | | | | | 4.0 Hz, 3H). | | | | | | |
| 495 | 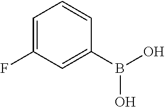 | 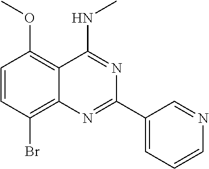 | 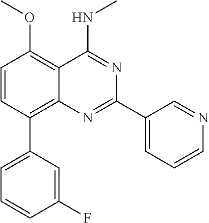 [SMILES: CNc1nc(-c2cccnc2)nc2c(-c3cccc(F)c3)ccc(OC)c12] | 2HCl | 1H NMR (300 MHz, DMSO) δ 9.47 (s, 1H), 8.73-8.46 (m, 3H), 7.78 (d, J = 8.4 Hz, 1H), 7.66-7.39 (m, 4H), 7.29-7.15 (m, 1H), 7.10 (d, J = 8.4 Hz, 1H), 4.05 (s, 3H), 3.17 (d, J = 4.3 Hz, 3H). | DMSO | 99 | Method R2 | 361 (M + 1) | 2.07 | Method D |
| 496 | 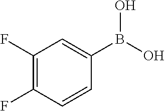 [SMILES: OB(O)c1ccc(F)c(F)c1] | 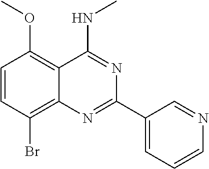 [SMILES: CNc1nc(-c2cccnc2)nc2c(Br)ccc(OC)c12] | 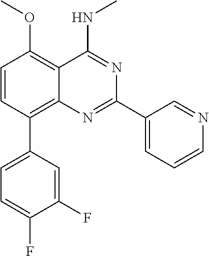 [SMILES: CNc1nc(-c2cccnc2)nc2c(-c3ccc(F)c(F)c3)ccc(OC)c12] | 2HCl | 1H NMR (300 MHz, cd3od) δ 9.52 (s, 1H), 9.35 (d, J = 7.9 Hz, 1H), 8.90 (d, J = 5.1 Hz, 1H), 8.13 (dd, J = 7.8, 6.0 Hz, 1H), 7.79 (d, J = 8.5 Hz, 1H), 7.69- 7.56 (m, 1H), 7.51-7.31 (m, 2H), 7.20 (d, J = 8.5 Hz, 1H), 4.15 (s, 3H), 3.31 (s, 3H). | CD3OD | 99 | Method R2 | 379 (M + 1) | 2.44 | Method C |
| 497 | 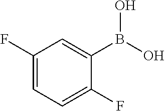 [SMILES: OB(O)c1cc(F)ccc1F] | 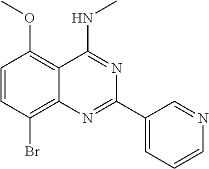 [SMILES: CNc1nc(-c2cccnc2)nc2c(Br)ccc(OC)c12] | 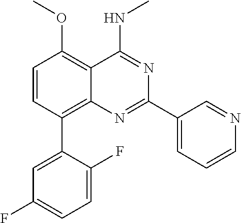 [SMILES: CNc1nc(-c2cccnc2)nc2c(-c3cc(F)ccc3F)ccc(OC)c12] | | 1H NMR (300 MHz, Acetone) δ 9.57-9.50 (m, 1H), 8.69-8.56 (m, 2H), 8.50 (s, 1H), 7.74 (dd, J = 8.4, 1.1 Hz, 1H), 7.46-7.17 (m, 4H), 7.12 (d, J = 8.4 Hz, 1H), 4.15 (s, 3H), 3.26 (d, J = 4.7 Hz, 3H). | Acetone | 99 | Method R2 | 379 (M + 1) | 2.32 | Method C |
| 498 | 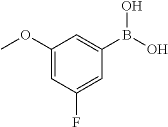 | 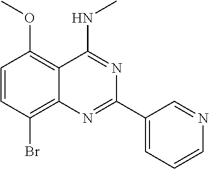 | 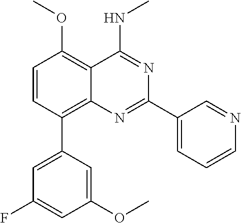 [SMILES: CNc1nc(-c2cccnc2)nc2c(-c3cc(F)cc(OC)c3)ccc(OC)c12] | | 1H NMR (300 MHz, Acetone) δ 9.65 (d, J = 1.3 Hz, 1H), 8.79-8.69 (m, 1H), 8.63 (d, J = 4.7 Hz, 1H), 8.51 (s, 1H), 7.78 (d, J = 8.4 Hz, 1H), 7.44 (dd, J = 7.8, 5.0 Hz, 1H), 7.21-7.05 (m, 3H), 6.81-6.68 (m, 1H), 4.12 (s, 3H), 3.89 (s, 3H), 3.25 (d, J = 4.7 Hz, 3H). | Acetone | 99 | Method R2 | 391 (M + 1) | 2.39 | Method C |

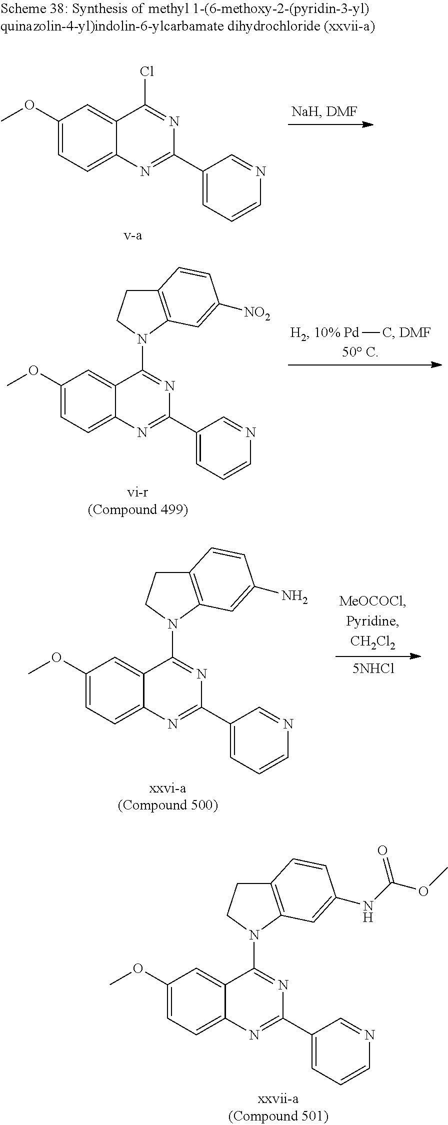

### 6-methoxy-4-(6-nitroindolin-1-yl)-2-(pyridin-3-yl)quinazoline (vi-r, compound 499)

6-methoxy-4-(6-nitroindolin-1-yl)-2-(pyridin-3-yl)quinazoline was synthesized in a similar method as described for 6-methoxy-2-(pyridin-3-yl)-4-(1H-pyrrolo[3,2-c]pyridin-1-yl)quinazoline using Method G2 in Scheme 8, substituting 6-nitroindoline for 1H-pyrrolo[3,2-c]pyridine to afford 6-methoxy-4-(6-nitroindolin-1-yl)-2-(pyridin-3-yl)quinazoline (0.35 g, 67.0%) as a pale yellow solid. ¹H NMR (400 MHz, DMSO) δ 9.56 (d, J=1.6 Hz, 1H), 8.77-8.63 (m, 2H), 8.47 (d, J=2.1 Hz, 1H), 8.01-7.88 (m, 2H), 7.67-7.50 (m, 3H), 7.47 (d, J=2.7 Hz, 1H), 4.73 (t, J=8.2 Hz, 2H), 3.90 (s, 3H), 3.37 (t, J=8.1 Hz, 2H), 3.33 (s, 2H).

### 1-(6-methoxy-2-(pyridin-3-yl)quinazolin-4-yl)indolin-6-amine (xxvi-a, compound 500)

To a solution of 6-methoxy-4-(6-nitroindolin-1-yl)-2-(pyridin-3-yl)quinazoline (0.30 g, 0.751 mmol) in DMF was added 10% Pd—C (0.1 g), and the mixture was stirred for 5 hr at 50° C. under a H₂ atmosphere. The reaction mixture was filtered to remove the catalyst. To the filtrate was added ethyl acetate (50 mL) which was washed with H₂O (30 ml×2) and brine. The organic layer was dried over Na₂SO₄, filtered and concentrated to give 1-(6-methoxy-2-(pyridin-3-yl)quinazolin-4-yl)indolin-6-amine (0.25 g, 0.565 mmol, 75% yield) as a brown powder. ¹H NMR (400 MHz, CDCl₃) δ 9.73 (dd, J=2.1, 0.7 Hz, 1H), 8.85-8.75 (m, 1H), 8.75-8.64 (m, 1H), 7.96 (d, J=9.2 Hz, 1H), 7.53-7.44 (m, 1H), 7.44-7.32 (m, 1H), 7.32-7.24 (m, 2H), 7.06 (d, J=7.9 Hz, 1H), 6.44-6.34 (m, 1H), 6.30 (dd, J=7.9, 2.1 Hz, 1H), 4.47 (t, J=7.9 Hz, 2H), 3.82 (s, 3H), 3.69-3.44 (m, 2H), 3.14 (t, J=7.8 Hz, 2H).

### Methyl 1-(6-methoxy-2-(pyridin-3-yl)quinazolin-4-yl)indolin-6-ylcarbamate dihydrochloride (xxvii-a, compound 501)

To a solution of 1-(6-methoxy-2-(pyridin-3-yl)quinazolin-4-yl)indolin-6-amine (0.30 g, 0.812 mmol) and pyridine (0.131 ml, 1.624 mmol) in CH₂Cl₂ (5 ml) was added chloroformic acid methyl ester (0.092 g, 0.975 mmol) dropwise at 0° C. The mixture was stirred for 2 h and then H₂O was added, the reaction mixture was concentrated down to give a suspension that was filtered. The precipciate was washed with H₂O and ether to give a yellow powder which was treated with a small excess of 5N HCl (1.0 mL) and washed with hot isopropyl alcohol to give methyl 1-(6-methoxy-2-(pyridin-3-yl)quinazolin-4-yl)indolin-6-ylcarbamate dihydrochloride (0.24 g, 0.48 mmol, 59.1% yield) as a pale brown powder. ¹H NMR (400 MHz, DMSO) δ 9.78 (s, 1H), 9.65 (d, J=1.6 Hz, 1H), 9.37 (d, J=8.2 Hz, 1H), 8.98 (d, J=4.4 Hz, 1H), 8.15-8.03 (m, 3H), 7.67 (dd, J=9.2, 2.7 Hz, 1H), 7.53 (d, J=2.6 Hz, 1H), 7.27 (d, J=8.1 Hz, 1H), 7.11 (dd, J=8.1, 1.7 Hz, 1H), 4.66 (t, J=7.8 Hz, 2H), 3.91 (s, 3H), 3.68 (s, 3H), 3.16 (t, J=7.7 Hz, 2H).

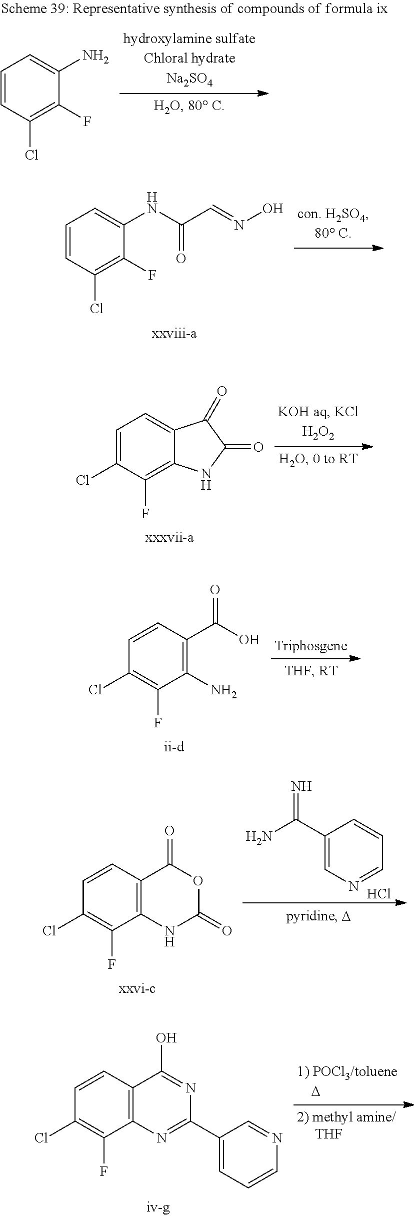

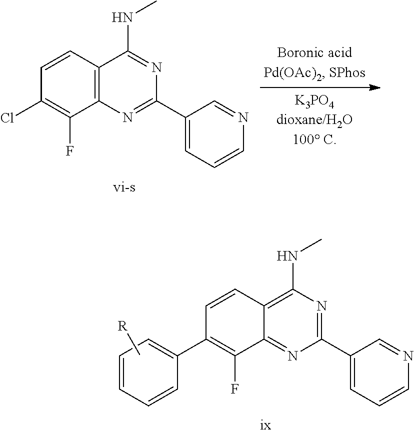

### Synthesis of (E)-N-(3-chloro-2-fluorophenyl)-2-(hydroxyimino)acetamide (xxviii-a)

Chloral hydrate (34.1 g, 206 mmol) was dissolved in water (300 mL) and sodium sulfate (137 g, 962 mmol) was added. To the suspension were added 3-chloro-2-fluoroaniline (20 g, 137 mmol), hydroxylamine sulfate (113 g, 687 mmol), sat. HCl (50 ml) and water (100 mL). The mixture was stirred at 80° C. for 3 h. The resultant solid was collected, washed with H₂O, and dried in an oven at 60° C. overnight. 32.81 g of the desired product was obtained. ¹H NMR (400 MHz, DMSO) δ 12.37 (s, 1H), 10.01 (s, 1H), 7.79 (dd, J=11.1, 4.1 Hz, 1H), 7.74 (s, 1H), 7.45-7.37 (m, 1H), 7.27-7.18 (m, 1H).

### 6-Chloro-7-fluoroindoline-2,3-dione (xxxvii-a)

(E)-N-(3-chloro-2-fluorophenyl)-2-(hydroxyimino)acetamide (5 g, 23.08 mmol) was added to a concentrated solution of H₂SO₄ (10 ml) at 55° C. The mixture was stirred at 80° C. for 30 min and then cooled to room temperature. The mixture was poured over ice and the precipitate was collected, washed with H₂O, and dried in vacuo to give 3.85 g of 6-chloro-7-fluoroindoline-2,3-dione. ¹H NMR (400 MHz, DMSO) δ 11.77 (s, 1H), 7.46-7.31 (m, 1H), 7.31-7.11 (m, 2H).

### 2-Amino-4-chloro-3-fluorobenzoic acid (ii-d)

To a suspension of 6-chloro-7-fluoroindoline-2,3-dione (3.85 g, 19.29 mmol) in water (5 ml) was added 1N-KOH aq. (38.6 ml, 38.6 mmol) at 0° C. Potassium chloride (4.31 g, 57.9 mmol) was added followed by careful addition of hydrogen peroxide (3.94 ml, 38.6 mmol) at 0° C. The mixture was stirred at room temperature for 1 h. Acetic acid (2.288 ml, 40 mmol) was added to the reaction mixture at 0° C. and the resulting solid was collected, washed with H₂O, and dried in an oven at 50° C. overnight to give 1.98 g of 2-Amino-4-chloro-3-fluorobenzoic acid. ¹H NMR (400 MHz, DMSO) δ 7.55 (dd, J=8.8, 1.8 Hz, 1H), 6.78 (br, 2H), 6.65 (dt, J=19.3, 9.7 Hz, 1H). 1H of carboxylic acid was not observed.

### 7-Chloro-8-fluoro-1H-benzo[d][1,3]oxazine-2,4-dione (xxvi-c)

To a suspension of 6-chloro-7-fluoroindoline-2,3-dione (1.98 g, 10.48 mmol) in THF (60 ml) under N₂, was added triphosgene (1.244 g, 4.19 mmol) at 0° C. The mixture was stirred at room temperature for 1 h 30 min. The reaction mixture was concentrated down to give a solid residue which was triturated with diethyl ether at room temperature. The resultant solid was collected, dried in vacuo to give 1.76 g of the desired product. ¹H NMR (400 MHz, DMSO) δ 12.18 (s, 1H), 7.76 (dd, J=8.6, 1.5 Hz, 1H), 7.42 (dd, J=8.6, 6.4 Hz, 1H).

### 7-Chloro-8-fluoro-2-(pyridin-3-yl)quinazolin-4-ol (iv-g)

To a solution of 7-chloro-8-fluoro-1H-benzo[d][1,3]oxazine-2,4-dione (1.76 g, 8.16 mmol) in pyridine (60 ml) under N₂ was added pyridine-3-carboximidamide hydrochloride (1.55 g, 9.83 mmol). The mixture was stirred at 115° C. for 3 h. The reaction mixture was concentrated down to give the crude product. The product was mixed with 1 N HCl aq. in methanol. The resultant solid was collected, washed with methanol, and dried in an oven at 60° C. for 2 days to give 1.39 g of the desired product. ¹H NMR (400 MHz, DMSO) δ 13.07 (s, 1H), 9.30 (d, J=2.3 Hz, 1H), 8.81 (dd, J=4.8, 1.5 Hz, 1H), 8.61-8.43 (m, 1H), 7.98 (dd, J=8.7, 1.4 Hz, 1H), 7.75-7.66 (m, 1H), 7.66-7.57 (m, 1H).

### 7-Chloro-8-fluoro-N-methyl-2-(pyridin-3-yl)quinazolin-4-amine (vi-s)

7-chloro-8-fluoro-2-(pyridin-3-yl)quinazolin-4-ol (1.39 g, 5.04 mmol) was suspended in toluene (50 ml) and POCl₃ (5 ml, 53.6 mmol) was added at room temperature. The mixture was refluxed for 4 h 30 min and subsequently concentrated down. The solid obtained was suspended in THF (100 ml) and an aqueous solution of methylamine (10 ml, 120 mmol) was added at 0° C. The mixture was heated to 50° C. for 1 h. The solution was concentrated down to give a solid. The crude material was stirred in water at room temperature for 2 days, then it was filtered to give 1.32 g of 7-Chloro-8-fluoro-N-methyl-2-(pyridin-3-yl)quinazolin-4-amine. ¹H NMR (400 MHz, DMSO) δ 9.62 (dd, J=2.1, 0.8 Hz, 1H), 8.79-8.73 (m, 2H), 8.71 (dd, J=4.8, 1.7 Hz, 1H), 8.07 (dd, J=9.0, 1.5 Hz, 1H), 7.67 (dd, J=8.9, 6.9 Hz, 1H), 7.56 (ddd, J=8.0, 4.8, 0.8 Hz, 1H), 3.16 (d, J=4.5 Hz, 3H).

### 8-fluoro-7-(4-fluorophenyl)-N-methyl-2-(pyridin-3-yl)quinazolin-4-amine (ix-k, compound 503)

8-fluoro-7-(4-fluorophenyl)-N-methyl-2-(pyridin-3-yl)quinazolin-4-amine was prepared from 7-chloro-8-fluoro-N-methyl-2-(pyridin-3-yl)quinazolin-4-amine and 4-fluorophenylboronic acid in a manner analogous to that described for tert-butyl 6-(4-(3-fluorooxetan-3-yl)-2-methylphenyl)-2-(pyridin-3-yl)quinazolin-4-yl(methyl)carbamate using Method R5 substituting 4-fluorophenylboronic acid for 3-(4-chloro-3-methylphenyl)-3-fluorooxetane.

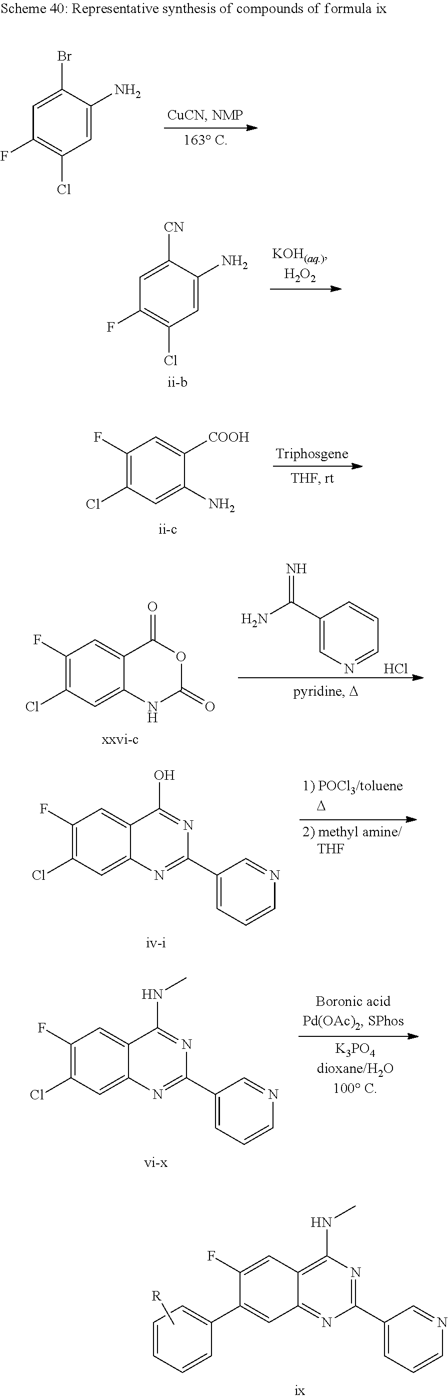

### 2-Amino-4-chloro-5-fluorobenzonitrile (ii-b)

To a solution of 2-bromo-5-chloro-4-fluoroaniline (synthesized according to the procedure of Tetrahedron Lett., 2002, 43, 7581-7583, 5.19 g, 23.12 mmol) in NMP (50 mL) under N₂ was added cuprous cyanide (4.14 g, 46.2 mmol) at room temperature. The reaction mixture was stirred at 163° C. for 5 h 30 min, and then poured into a cold aqueous solution of NH₄OH (100 ml) and stirred overnight at room temperature. The resulting precipitate was filtered and washed with water. The obtained solid was dissolved in CH₂Cl₂ and remaining solid was filtered off. The filtrate was concentrated to give the crude product which was purified by silica-gel column chromatography to give 2.80 g of 2-amino-4-chloro-5-fluorobenzonitrile. ¹H NMR (400 MHz, DMSO) δ 7.61 (d, J=9.3 Hz, 1H), 6.93 (t, J=8.5 Hz, 1H), 6.21 (s, 2H).

### 2-amino-4-chloro-5-fluorobenzoic acid (ii-c)

To a suspension of 2-amino-4-chloro-5-fluorobenzonitrile (2.92 g, 17.12 mmol) in 1 N KOH_{(aq.)} (56 mL) was added hydrogen peroxide (4 ml, 39.2 mmol) and heated to 130° C. for 3 h. The reaction mixture was diluted with water (200 ml), followed by addition of 5N HCl (ca. 12 mL) at 0° C. until a precipitate appeared. The suspension was stirred at room temperature overnight. The solid was filtered, washed with water and dried in vacuo to give 2.47 g of 2-amino-4-chloro-5-fluorobenzoic acid. ¹H NMR (400 MHz, DMSO) δ 7.55 (d, J=10.3 Hz, 1H), 6.93 (d, J=6.5 Hz, 1H). The protons of the aniline and carboxylic acid were not observed.

### 7-chloro-6-fluoro-N-methyl-2-(pyridin-3-yl)quinazolin-4-amine (vi-x)

7-chloro-6-fluoro-N-methyl-2-(pyridin-3-yl)quinazolin-4-amine was synthesized in a similar manner to described in Scheme 39 for 7-chloro-8-fluoro-N-methyl-2-(pyridin-3-yl)quinazolin-4-amine substituting 2-amino-4-chloro-5-fluorobenzoic acid for 2-amino-4-chloro-3-fluorobenzoic acid. The reaction was concentrated down and triturated with water to obtain 2.54 g of 7-chloro-6-fluoro-N-methyl-2-(pyridin-3-yl)quinazolin-4-amine. ¹H NMR (400 MHz, DMSO) δ 9.60 (d, J=1.5 Hz, 1H), 8.77-8.71 (m, 1H), 8.69 (dd, J=4.8, 1.7 Hz, 1H), 8.56 (d, J=4.4 Hz, 1H), 8.29 (d, J=10.2 Hz, 1H), 8.04 (d, J=7.3 Hz, 1H), 7.59-7.50 (m, 1H), 3.15 (d, J=4.4 Hz, 3H).

### 6-bromo-8-fluoro-N-methyl-2-(pyridin-3-yl)quinazolin-4-amine (vi-z)

6-bromo-8-fluoro-N-methyl-2-(pyridin-3-yl)quinazolin-4-amine was synthesized in a similar manner to described in Scheme 39 for 7-chloro-8-fluoro-N-methyl-2-(pyridin-3-yl)quinazolin-4-amine substituting 2-amino-5-bromo-3-fluorobenzoic acid hydrobromide for 2-amino-4-chloro-3-fluorobenzoic acid. The reaction was concentrated down and triturated with water to obtain 3.94 g of 6-bromo-8-fluoro-N-methyl-2-(pyridin-3-yl)quinazolin-4-amine. ¹H NMR (400 MHz, DMSO) δ 9.61 (d, J=1.4 Hz, 1H), 8.88-8.65 (m, 3H), 8.37 (s, 1H), 7.96 (dd, J=10.0, 1.9 Hz, 1H), 7.55 (dd, J=7.6, 5.1 Hz, 1H), 3.15 (d, J=4.5 Hz, 3H).

The compounds in the following table were prepared in a manner analogous to that described in Scheme 39 and 40

TABLE 10

| | Starting | Starting | |
|---|---|---|---|
| Number | Material 1 | Material 2 | Product |
| 504 | 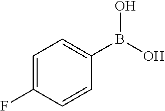 | 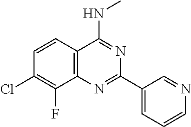 [SMILES: CNc1nc(-c2cccnc2)nc2c(F)c(Cl)ccc12] | 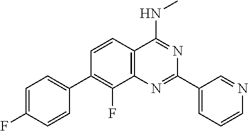 [SMILES: CNc1nc(-c2cccnc2)nc2c(F)c(-c3ccc(F)cc3)ccc12] |
| 505 | 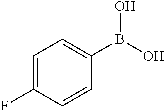 | 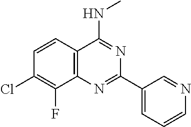 [SMILES: CNc1nc(-c2cccnc2)nc2c(F)c(Cl)ccc12] | 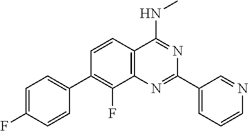 [SMILES: CNc1nc(-c2cccnc2)nc2c(F)c(-c3ccc(F)cc3)ccc12] |
| 506 | 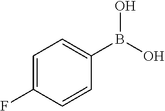 | 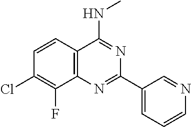 [SMILES: CNc1nc(-c2cccnc2)nc2c(F)c(Cl)ccc12] | 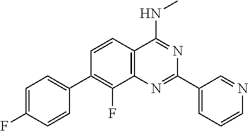 [SMILES: CNc1nc(-c2cccnc2)nc2c(F)c(-c3ccc(F)cc3)ccc12] |
| 507 | 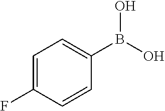 | 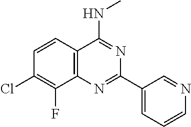 [SMILES: CNc1nc(-c2cccnc2)nc2c(F)c(Cl)ccc12] | 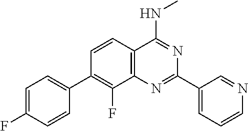 [SMILES: CNc1nc(-c2cccnc2)nc2c(F)c(-c3ccc(F)cc3)ccc12] |
| 508 | 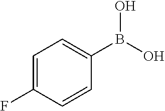 | 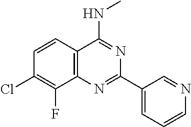 [SMILES: CNc1nc(-c2cccnc2)nc2c(F)c(Cl)ccc12] | 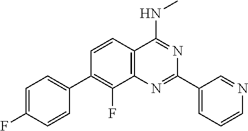 [SMILES: CNc1nc(-c2cccnc2)nc2c(F)c(-c3ccc(F)cc3)ccc12] |
| 509 | 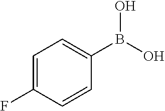 | 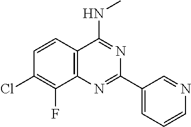 [SMILES: CNc1nc(-c2cccnc2)nc2c(F)c(Cl)ccc12] | 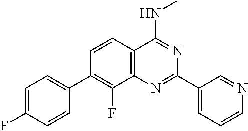 [SMILES: CNc1nc(-c2cccnc2)nc2c(F)c(-c3ccc(F)cc3)ccc12] |
| 510 | 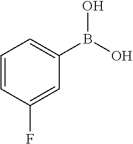 | 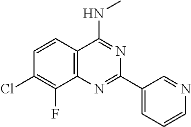 [SMILES: CNc1nc(-c2cccnc2)nc2c(F)c(Cl)ccc12] | 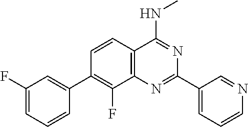 [SMILES: CNc1nc(-c2cccnc2)nc2c(F)c(-c3cccc(F)c3)ccc12] |
| 511 | 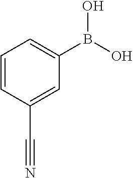 [SMILES: N#Cc1cccc(B(O)O)c1] | 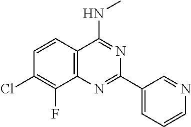 [SMILES: CNc1nc(-c2cccnc2)nc2c(F)c(Cl)ccc12] | 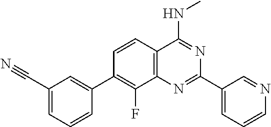 [SMILES: CNc1nc(-c2cccnc2)nc2c(F)c(-c3cccc(C#N)c3)ccc12] |
| 512 | 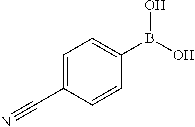 [SMILES: N#Cc1ccc(B(O)O)cc1] | 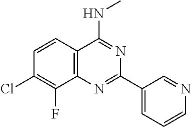 [SMILES: CNc1nc(-c2cccnc2)nc2c(F)c(Cl)ccc12] | 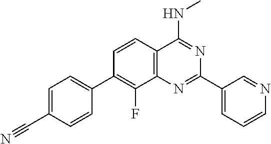 [SMILES: CNc1nc(-c2cccnc2)nc2c(F)c(-c3ccc(C#N)cc3)ccc12] |
| 513 | 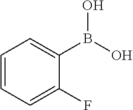 | 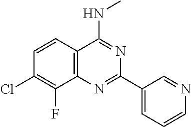 [SMILES: CNc1nc(-c2cccnc2)nc2c(F)c(Cl)ccc12] | 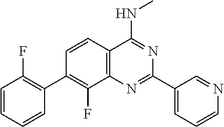 [SMILES: CNc1nc(-c2cccnc2)nc2c(F)c(-c3ccccc3F)ccc12] |
| 514 | 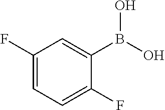 [SMILES: OB(O)c1cc(F)ccc1F] | 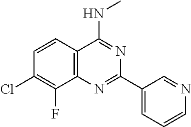 [SMILES: CNc1nc(-c2cccnc2)nc2c(F)c(Cl)ccc12] | 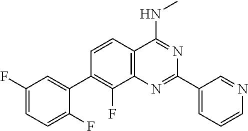 [SMILES: CNc1nc(-c2cccnc2)nc2c(F)c(-c3cc(F)ccc3F)ccc12] |
| 515 | 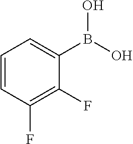 [SMILES: OB(O)c1cccc(F)c1F] | 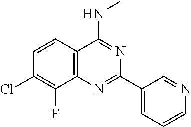 [SMILES: CNc1nc(-c2cccnc2)nc2c(F)c(Cl)ccc12] | 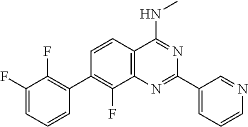 [SMILES: CNc1nc(-c2cccnc2)nc2c(F)c(-c3cccc(F)c3F)ccc12] |
| 516 | 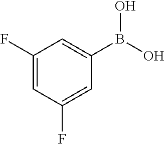 [SMILES: OB(O)c1cc(F)cc(F)c1] | 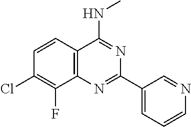 [SMILES: CNc1nc(-c2cccnc2)nc2c(F)c(Cl)ccc12] | 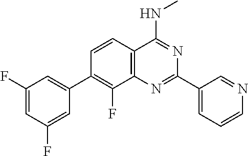 [SMILES: CNc1nc(-c2cccnc2)nc2c(F)c(-c3cc(F)cc(F)c3)ccc12] |
| 517 | 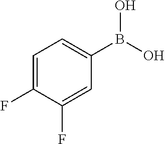 [SMILES: OB(O)c1ccc(F)c(F)c1] | 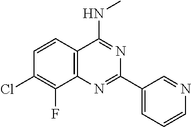 [SMILES: CNc1nc(-c2cccnc2)nc2c(F)c(Cl)ccc12] | 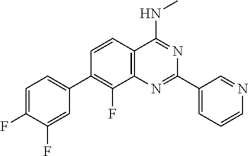 [SMILES: CNc1nc(-c2cccnc2)nc2c(F)c(-c3ccc(F)c(F)c3)ccc12] |
| 518 | 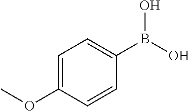 | 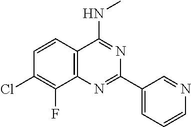 [SMILES: CNc1nc(-c2cccnc2)nc2c(F)c(Cl)ccc12] | 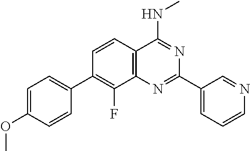 [SMILES: CNc1nc(-c2cccnc2)nc2c(F)c(-c3ccc(OC)cc3)ccc12] |
| 519 | 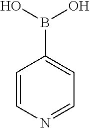 | 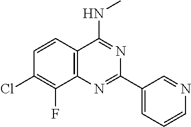 [SMILES: CNc1nc(-c2cccnc2)nc2c(F)c(Cl)ccc12] | 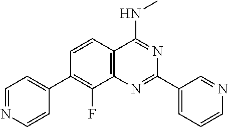 [SMILES: CNc1nc(-c2cccnc2)nc2c(F)c(-c3ccncc3)ccc12] |
| 520 | 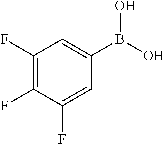 [SMILES: OB(O)c1cc(F)c(F)c(F)c1] | 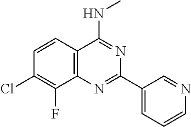 [SMILES: CNc1nc(-c2cccnc2)nc2c(F)c(Cl)ccc12] | 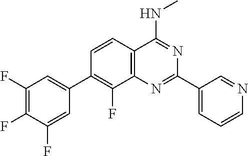 [SMILES: CNc1nc(-c2cccnc2)nc2c(F)c(-c3cc(F)c(F)c(F)c3)ccc12] |
| 521 | 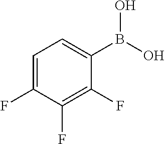 [SMILES: OB(O)c1ccc(F)c(F)c1F] | 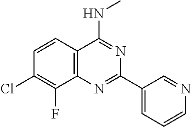 [SMILES: CNc1nc(-c2cccnc2)nc2c(F)c(Cl)ccc12] | 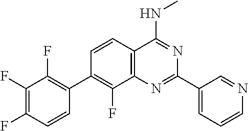 [SMILES: CNc1nc(-c2cccnc2)nc2c(F)c(-c3ccc(F)c(F)c3F)ccc12] |
| 522 | 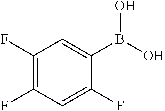 [SMILES: OB(O)c1cc(F)c(F)cc1F] | 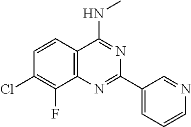 [SMILES: CNc1nc(-c2cccnc2)nc2c(F)c(Cl)ccc12] | 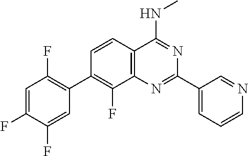 [SMILES: CNc1nc(-c2cccnc2)nc2c(F)c(-c3cc(F)c(F)cc3F)ccc12] |
| 523 | 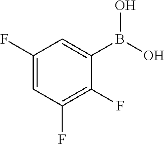 [SMILES: OB(O)c1cc(F)cc(F)c1F] | 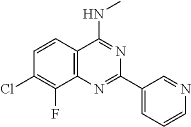 [SMILES: CNc1nc(-c2cccnc2)nc2c(F)c(Cl)ccc12] | 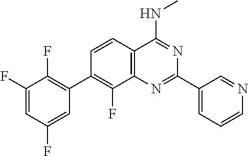 [SMILES: CNc1nc(-c2cccnc2)nc2c(F)c(-c3cc(F)cc(F)c3F)ccc12] |
| 524 | 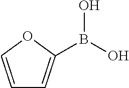 | 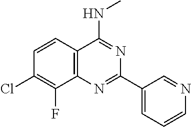 [SMILES: CNc1nc(-c2cccnc2)nc2c(F)c(Cl)ccc12] | 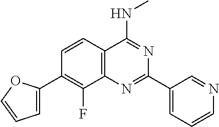 [SMILES: CNc1nc(-c2cccnc2)nc2c(F)c(-c3ccco3)ccc12] |
| 525 | 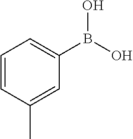 | 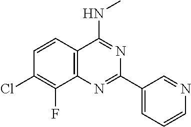 [SMILES: CNc1nc(-c2cccnc2)nc2c(F)c(Cl)ccc12] | 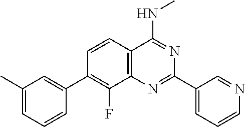 [SMILES: CNc1nc(-c2cccnc2)nc2c(F)c(-c3cccc(C)c3)ccc12] |
| 526 | 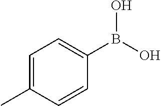 | 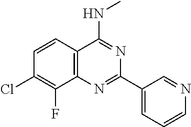 [SMILES: CNc1nc(-c2cccnc2)nc2c(F)c(Cl)ccc12] | 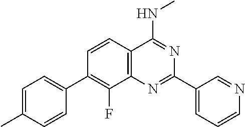 [SMILES: CNc1nc(-c2cccnc2)nc2c(F)c(-c3ccc(C)cc3)ccc12] |
| 527 | 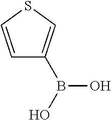 | 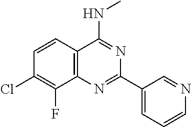 [SMILES: CNc1nc(-c2cccnc2)nc2c(F)c(Cl)ccc12] | 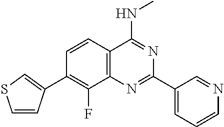 [SMILES: CNc1nc(-c2cccnc2)nc2c(F)c(-c3ccsc3)ccc12] |
| 528 | 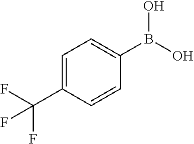 [SMILES: OB(O)c1ccc(C(F)(F)F)cc1] | 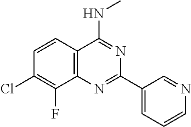 [SMILES: CNc1nc(-c2cccnc2)nc2c(F)c(Cl)ccc12] | 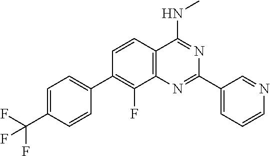 [SMILES: CNc1nc(-c2cccnc2)nc2c(F)c(-c3ccc(C(F)(F)F)cc3)ccc12] |
| 529 | 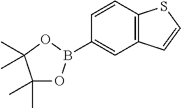 [SMILES: CC1(C)OB(c2ccc3sccc3c2)OC1(C)C] | 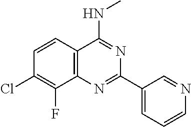 [SMILES: CNc1nc(-c2cccnc2)nc2c(F)c(Cl)ccc12] | 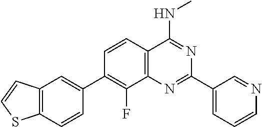 [SMILES: CNc1nc(-c2cccnc2)nc2c(F)c(-c3ccc4sccc4c3)ccc12] |
| 530 | 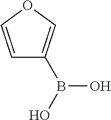 | 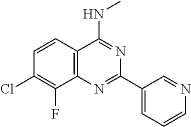 [SMILES: CNc1nc(-c2cccnc2)nc2c(F)c(Cl)ccc12] | 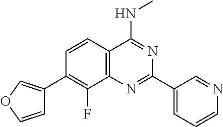 [SMILES: CNc1nc(-c2cccnc2)nc2c(F)c(-c3ccoc3)ccc12] |
| 531 | 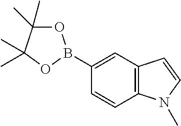 [SMILES: Cn1ccc2cc(B3OC(C)(C)C(C)(C)O3)ccc21] | 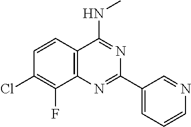 [SMILES: CNc1nc(-c2cccnc2)nc2c(F)c(Cl)ccc12] | 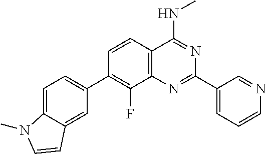 [SMILES: CNc1nc(-c2cccnc2)nc2c(F)c(-c3ccc4c(ccn4C)c3)ccc12] |
| 532 | 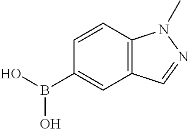 | 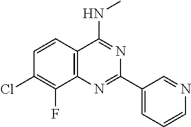 [SMILES: CNc1nc(-c2cccnc2)nc2c(F)c(Cl)ccc12] | 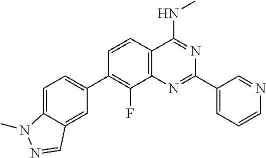 [SMILES: CNc1nc(-c2cccnc2)nc2c(F)c(-c3ccc4c(cnn4C)c3)ccc12] |
| 533 | 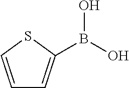 | 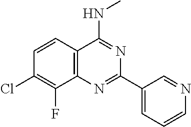 [SMILES: CNc1nc(-c2cccnc2)nc2c(F)c(Cl)ccc12] | 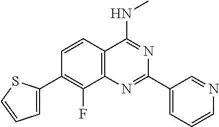 [SMILES: CNc1nc(-c2cccnc2)nc2c(F)c(-c3cccs3)ccc12] |
| 534 | 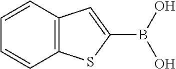 [SMILES: OB(O)c1cc2ccccc2s1] | 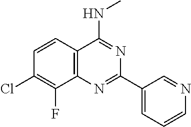 [SMILES: CNc1nc(-c2cccnc2)nc2c(F)c(Cl)ccc12] | 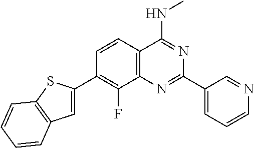 [SMILES: CNc1nc(-c2cccnc2)nc2c(F)c(-c3cc4ccccc4s3)ccc12] |
| 535 | 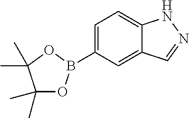 [SMILES: CC1(C)OB(c2ccc3[nH]ncc3c2)OC1(C)C] | 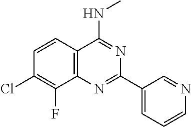 [SMILES: CNc1nc(-c2cccnc2)nc2c(F)c(Cl)ccc12] | 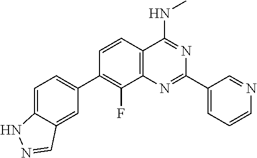 [SMILES: CNc1nc(-c2cccnc2)nc2c(F)c(-c3ccc4[nH]ncc4c3)ccc12] |
| 536 | 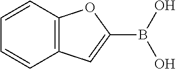 [SMILES: OB(O)c1cc2ccccc2o1] | 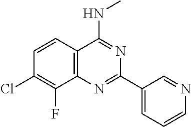 [SMILES: CNc1nc(-c2cccnc2)nc2c(F)c(Cl)ccc12] | 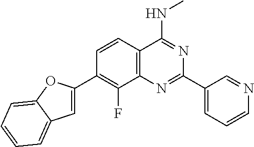 [SMILES: CNc1nc(-c2cccnc2)nc2c(F)c(-c3cc4ccccc4o3)ccc12] |
| 537 | 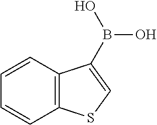 [SMILES: OB(O)c1csc2ccccc12] | 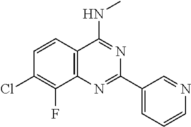 [SMILES: CNc1nc(-c2cccnc2)nc2c(F)c(Cl)ccc12] | 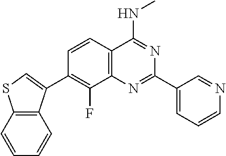 [SMILES: CNc1nc(-c2cccnc2)nc2c(F)c(-c3csc4ccccc34)ccc12] |
| 538 | 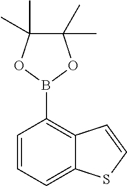 [SMILES: CC1(C)OB(c2cccc3sccc23)OC1(C)C] | 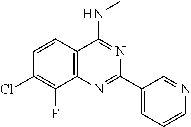 [SMILES: CNc1nc(-c2cccnc2)nc2c(F)c(Cl)ccc12] | 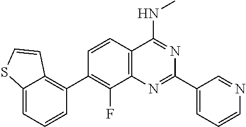 [SMILES: CNc1nc(-c2cccnc2)nc2c(F)c(-c3cccc4sccc34)ccc12] |
| 539 | 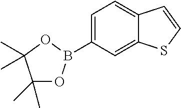 [SMILES: CC1(C)OB(c2ccc3ccsc3c2)OC1(C)C] | 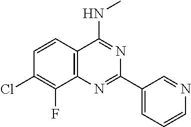 [SMILES: CNc1nc(-c2cccnc2)nc2c(F)c(Cl)ccc12] | 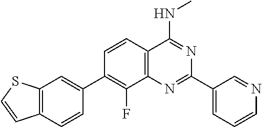 [SMILES: CNc1nc(-c2cccnc2)nc2c(F)c(-c3ccc4ccsc4c3)ccc12] |
| 540 | 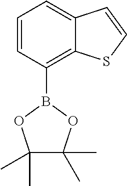 [SMILES: CC1(C)OB(c2cccc3ccsc23)OC1(C)C] | 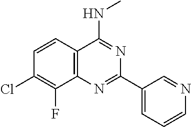 [SMILES: CNc1nc(-c2cccnc2)nc2c(F)c(Cl)ccc12] | 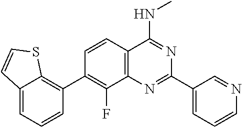 [SMILES: CNc1nc(-c2cccnc2)nc2c(F)c(-c3cccc4ccsc34)ccc12] |
| 541 | 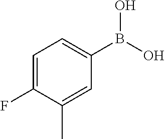 | 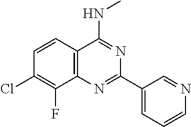 [SMILES: CNc1nc(-c2cccnc2)nc2c(F)c(Cl)ccc12] | 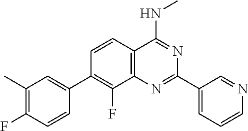 [SMILES: CNc1nc(-c2cccnc2)nc2c(F)c(-c3ccc(F)c(C)c3)ccc12] |
| 542 | 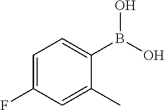 | 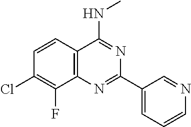 [SMILES: CNc1nc(-c2cccnc2)nc2c(F)c(Cl)ccc12] | 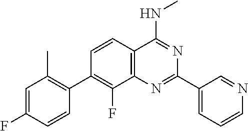 [SMILES: CNc1nc(-c2cccnc2)nc2c(F)c(-c3ccc(F)cc3C)ccc12] |
| 543 | 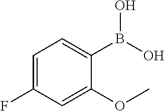 | 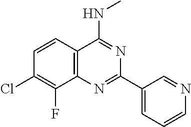 [SMILES: CNc1nc(-c2cccnc2)nc2c(F)c(Cl)ccc12] | 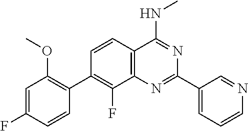 [SMILES: CNc1nc(-c2cccnc2)nc2c(F)c(-c3ccc(F)cc3OC)ccc12] |
| 544 | 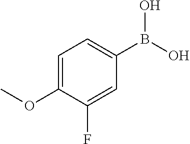 | 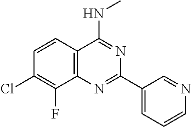 [SMILES: CNc1nc(-c2cccnc2)nc2c(F)c(Cl)ccc12] | 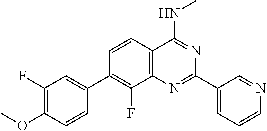 [SMILES: CNc1nc(-c2cccnc2)nc2c(F)c(-c3ccc(OC)c(F)c3)ccc12] |
| 545 | 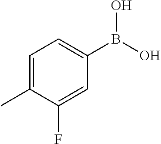 | 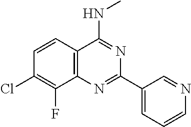 [SMILES: CNc1nc(-c2cccnc2)nc2c(F)c(Cl)ccc12] | 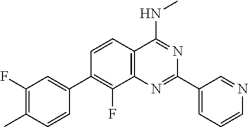 [SMILES: CNc1nc(-c2cccnc2)nc2c(F)c(-c3ccc(C)c(F)c3)ccc12] |
| 546 | 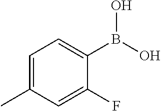 | 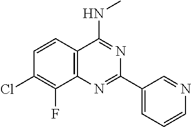 [SMILES: CNc1nc(-c2cccnc2)nc2c(F)c(Cl)ccc12] | 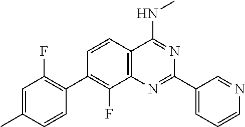 [SMILES: CNc1nc(-c2cccnc2)nc2c(F)c(-c3ccc(C)cc3F)ccc12] |
| 547 | 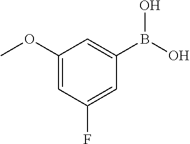 | 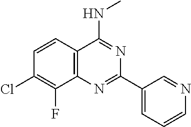 [SMILES: CNc1nc(-c2cccnc2)nc2c(F)c(Cl)ccc12] | 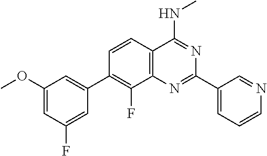 [SMILES: CNc1nc(-c2cccnc2)nc2c(F)c(-c3cc(F)cc(OC)c3)ccc12] |
| 548 | 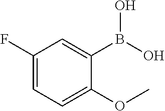 | 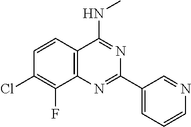 [SMILES: CNc1nc(-c2cccnc2)nc2c(F)c(Cl)ccc12] | 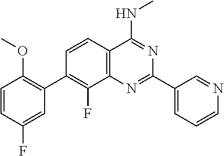 [SMILES: CNc1nc(-c2cccnc2)nc2c(F)c(-c3cc(F)ccc3OC)ccc12] |
| 549 | 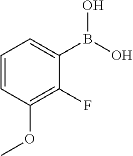 | 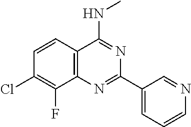 [SMILES: CNc1nc(-c2cccnc2)nc2c(F)c(Cl)ccc12] | 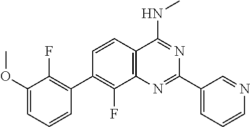 [SMILES: CNc1nc(-c2cccnc2)nc2c(F)c(-c3cccc(OC)c3F)ccc12] |
| 550 | 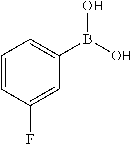 | 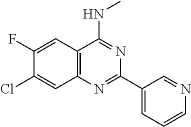 [SMILES: CNc1nc(-c2cccnc2)nc2cc(Cl)c(F)cc12] | 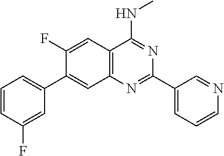 [SMILES: CNc1nc(-c2cccnc2)nc2cc(-c3cccc(F)c3)c(F)cc12] |
| 551 | 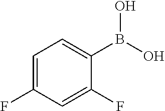 [SMILES: OB(O)c1ccc(F)cc1F] | 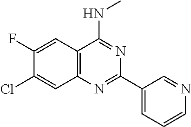 [SMILES: CNc1nc(-c2cccnc2)nc2cc(Cl)c(F)cc12] | 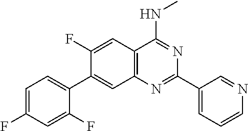 [SMILES: CNc1nc(-c2cccnc2)nc2cc(-c3ccc(F)cc3F)c(F)cc12] |
| 552 | 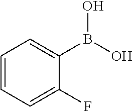 | 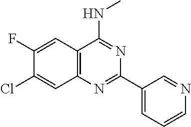 [SMILES: CNc1nc(-c2cccnc2)nc2cc(Cl)c(F)cc12] | 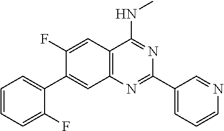 [SMILES: CNc1nc(-c2cccnc2)nc2cc(-c3ccccc3F)c(F)cc12] |
| 553 | 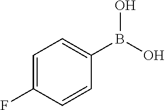 | 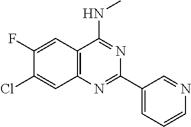 [SMILES: CNc1nc(-c2cccnc2)nc2cc(Cl)c(F)cc12] | 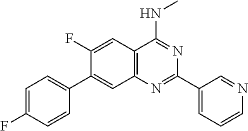 [SMILES: CNc1nc(-c2cccnc2)nc2cc(-c3ccc(F)cc3)c(F)cc12] |
| 554 | 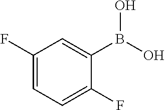 [SMILES: OB(O)c1cc(F)ccc1F] | 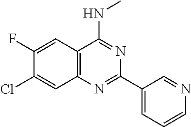 [SMILES: CNc1nc(-c2cccnc2)nc2cc(Cl)c(F)cc12] | 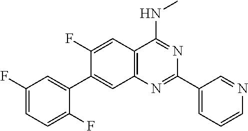 [SMILES: CNc1nc(-c2cccnc2)nc2cc(-c3cc(F)ccc3F)c(F)cc12] |
| 555 | 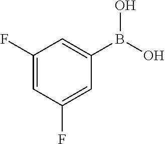 [SMILES: OB(O)c1cc(F)cc(F)c1] | 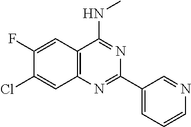 [SMILES: CNc1nc(-c2cccnc2)nc2cc(Cl)c(F)cc12] | 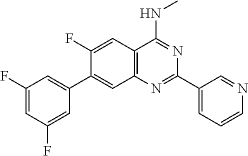 [SMILES: CNc1nc(-c2cccnc2)nc2cc(-c3cc(F)cc(F)c3)c(F)cc12] |
| 556 | 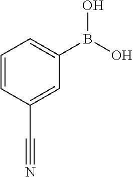 [SMILES: N#Cc1cccc(B(O)O)c1] | 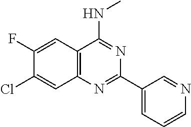 [SMILES: CNc1nc(-c2cccnc2)nc2cc(Cl)c(F)cc12] | 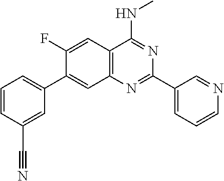 [SMILES: CNc1nc(-c2cccnc2)nc2cc(-c3cccc(C#N)c3)c(F)cc12] |
| 557 | 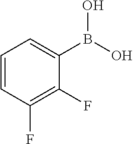 [SMILES: OB(O)c1cccc(F)c1F] | 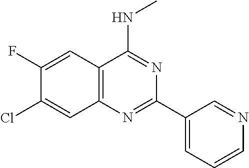 [SMILES: CNc1nc(-c2cccnc2)nc2cc(Cl)c(F)cc12] | 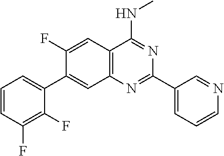 [SMILES: CNc1nc(-c2cccnc2)nc2cc(-c3cccc(F)c3F)c(F)cc12] |
| 558 | 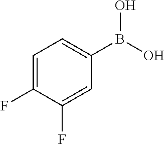 [SMILES: OB(O)c1ccc(F)c(F)c1] | 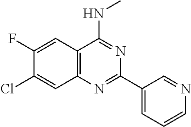 [SMILES: CNc1nc(-c2cccnc2)nc2cc(Cl)c(F)cc12] | 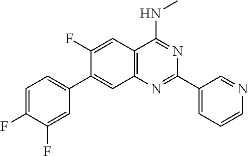 [SMILES: CNc1nc(-c2cccnc2)nc2cc(-c3ccc(F)c(F)c3)c(F)cc12] |
| 559 | 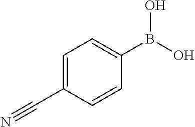 [SMILES: N#Cc1ccc(B(O)O)cc1] | 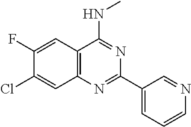 [SMILES: CNc1nc(-c2cccnc2)nc2cc(Cl)c(F)cc12] | 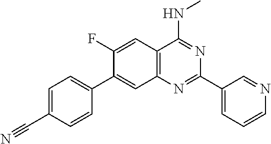 [SMILES: CNc1nc(-c2cccnc2)nc2cc(-c3ccc(C#N)cc3)c(F)cc12] |
| 560 | 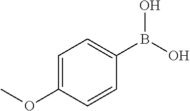 | 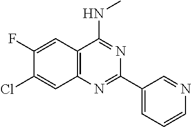 [SMILES: CNc1nc(-c2cccnc2)nc2cc(Cl)c(F)cc12] | 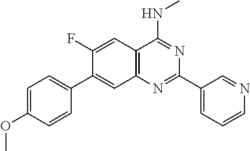 [SMILES: CNc1nc(-c2cccnc2)nc2cc(-c3ccc(OC)cc3)c(F)cc12] |
| 561 | 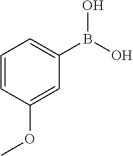 | 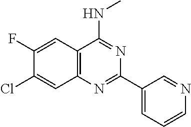 [SMILES: CNc1nc(-c2cccnc2)nc2cc(Cl)c(F)cc12] | 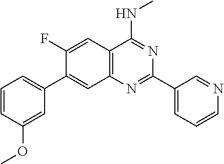 [SMILES: CNc1nc(-c2cccnc2)nc2cc(-c3cccc(OC)c3)c(F)cc12] |
| 562 | 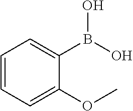 | 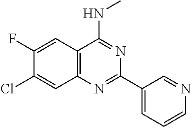 [SMILES: CNc1nc(-c2cccnc2)nc2cc(Cl)c(F)cc12] | 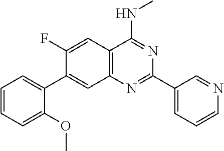 [SMILES: CNc1nc(-c2cccnc2)nc2cc(-c3ccccc3OC)c(F)cc12] |
| 563 | 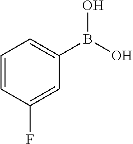 | 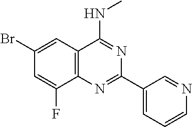 [SMILES: CNc1nc(-c2cccnc2)nc2c(F)cc(Br)cc12] | 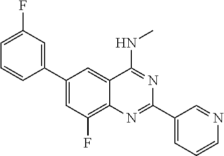 [SMILES: CNc1nc(-c2cccnc2)nc2c(F)cc(-c3cccc(F)c3)cc12] |
| 564 | 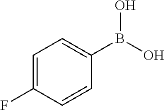 | 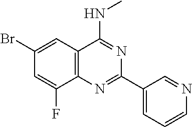 [SMILES: CNc1nc(-c2cccnc2)nc2c(F)cc(Br)cc12] | 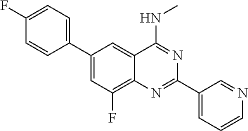 [SMILES: CNc1nc(-c2cccnc2)nc2c(F)cc(-c3ccc(F)cc3)cc12] |
| 565 | 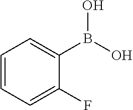 | 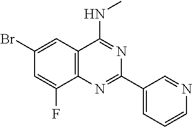 [SMILES: CNc1nc(-c2cccnc2)nc2c(F)cc(Br)cc12] | 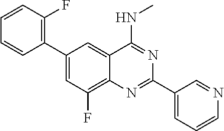 [SMILES: CNc1nc(-c2cccnc2)nc2c(F)cc(-c3ccccc3F)cc12] |
| 566 | 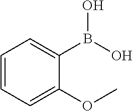 | 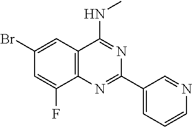 [SMILES: CNc1nc(-c2cccnc2)nc2c(F)cc(Br)cc12] | 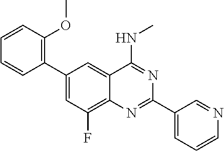 [SMILES: CNc1nc(-c2cccnc2)nc2c(F)cc(-c3ccccc3OC)cc12] |
| 567 | 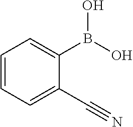 [SMILES: N#Cc1ccccc1B(O)O] | 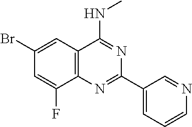 [SMILES: CNc1nc(-c2cccnc2)nc2c(F)cc(Br)cc12] | 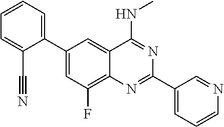 [SMILES: CNc1nc(-c2cccnc2)nc2c(F)cc(-c3ccccc3C#N)cc12] |
| 568 | 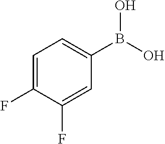 [SMILES: OB(O)c1ccc(F)c(F)c1] | 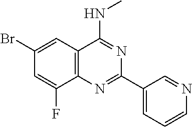 [SMILES: CNc1nc(-c2cccnc2)nc2c(F)cc(Br)cc12] | 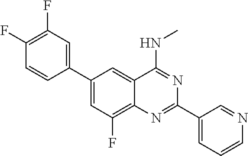 [SMILES: CNc1nc(-c2cccnc2)nc2c(F)cc(-c3ccc(F)c(F)c3)cc12] |
| 569 | 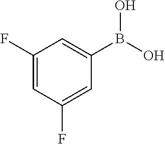 [SMILES: OB(O)c1cc(F)cc(F)c1] | 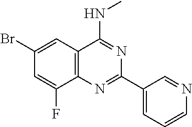 [SMILES: CNc1nc(-c2cccnc2)nc2c(F)cc(Br)cc12] | 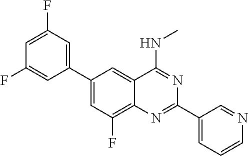 [SMILES: CNc1nc(-c2cccnc2)nc2c(F)cc(-c3cc(F)cc(F)c3)cc12] |
| 570 | 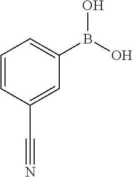 [SMILES: N#Cc1cccc(B(O)O)c1] | 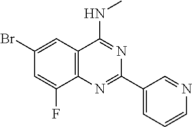 [SMILES: CNc1nc(-c2cccnc2)nc2c(F)cc(Br)cc12] | 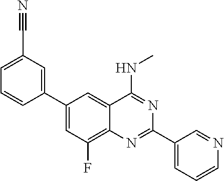 [SMILES: CNc1nc(-c2cccnc2)nc2c(F)cc(-c3cccc(C#N)c3)cc12] |
| 571 | 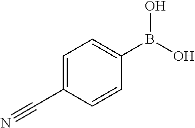 [SMILES: N#Cc1ccc(B(O)O)cc1] | 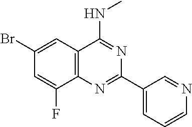 [SMILES: CNc1nc(-c2cccnc2)nc2c(F)cc(Br)cc12] | 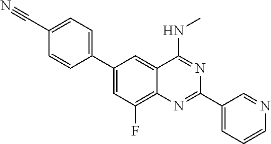 [SMILES: CNc1nc(-c2cccnc2)nc2c(F)cc(-c3ccc(C#N)cc3)cc12] |
| 572 | 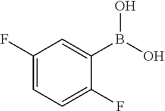 [SMILES: OB(O)c1cc(F)ccc1F] | 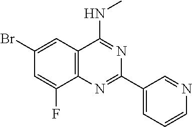 [SMILES: CNc1nc(-c2cccnc2)nc2c(F)cc(Br)cc12] | 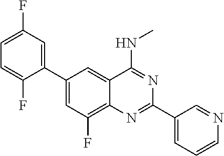 [SMILES: CNc1nc(-c2cccnc2)nc2c(F)cc(-c3cc(F)ccc3F)cc12] |
| 573 | 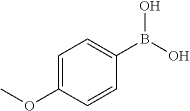 | 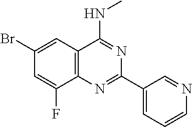 [SMILES: CNc1nc(-c2cccnc2)nc2c(F)cc(Br)cc12] | 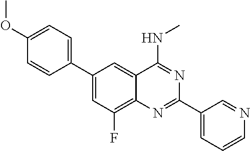 [SMILES: CNc1nc(-c2cccnc2)nc2c(F)cc(-c3ccc(OC)cc3)cc12] |
| 574 | 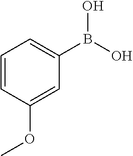 | 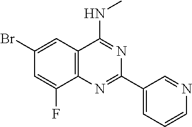 [SMILES: CNc1nc(-c2cccnc2)nc2c(F)cc(Br)cc12] | 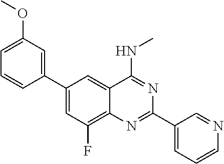 [SMILES: CNc1nc(-c2cccnc2)nc2c(F)cc(-c3cccc(OC)c3)cc12] |
| 575 | 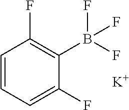 | 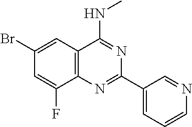 [SMILES: CNc1nc(-c2cccnc2)nc2c(F)cc(Br)cc12] | 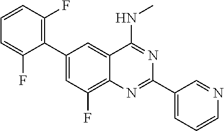 [SMILES: CNc1nc(-c2cccnc2)nc2c(F)cc(-c3c(F)cccc3F)cc12] |

| | Salt | | ¹H NMR | Purity | | | Retention | LCMS |
|---|---|---|---|---|---|---|---|---|
| Number | type | ¹H NMR | Solvent | percent | Method of Coupling | LCMS | Time | Method |
| 504 | HCl | ¹H NMR (400 MHz, DMSO) δ 9.68 | DMSO | >98 | Method | | | |
| | | (s, 1H), 9.24 (d, J = 8.0 Hz, 1H), | | | R5 | | | |
| | | 8.93 (t, J = 5.5 Hz, 2H), 8.20 (d, J = | | | | | | |
| | | 8.6 Hz, 1H), 8.01 (dd, J = 7.9, | | | | | | |
| | | 5.4 Hz, 1H), 7.77 (dt, J = 18.2, 9.1 | | | | | | |
| | | Hz, 2H), 7.73-7.63 (m, 1H), 7.41 | | | | | | |
| | | (t, J = 8.9 Hz, 2H), 3.21 (d, J = | | | | | | |
| | | 4.4 Hz, 3H). 1H of HCl was not | | | | | | |
| | | observed. | | | | | | |
| 505 | 2HCl | ¹H NMR (400 MHz, DMSO) δ 9.69 | DMSO | >98 | Method | | | |
| | | (d, J = 1.8 Hz, 1H), 9.31 (d, J = | | | R5 | | | |
| | | 7.9 Hz, 1H), 8.96 (t, J = 5.3 Hz, | | | | | | |
| | | 2H), 8.21 (d, J = 8.6 Hz, 1H), 8.07 | | | | | | |
| | | (dd, J = 7.9, 5.5 Hz, 1H), 7.78 | | | | | | |
| | | (dd, J = 7.8, 5.6 Hz, 2H), 7.75- | | | | | | |
| | | 7.64 (m, 1H), 7.51-7.30 (m, 2H), | | | | | | |
| | | 3.21 (d, J = 4.5 Hz, 3H). 1H of | | | | | | |
| | | 2HCl was not observed | | | | | | |
| 506 | MsOH | ¹H NMR (400 MHz, DMSO) δ 9.69 | DMSO | >98 | Method | | | |
| | | (d, J = 1.6 Hz, 1H), 9.11 (d, J = | | | R5 | | | |
| | | 8.1 Hz, 1H), 8.87 (dd, J = 5.2, 1.5 | | | | | | |
| | | Hz, 1H), 8.81 (d, J = 4.6 Hz, 1H), | | | | | | |
| | | 8.16 (d, J = 8.6 Hz, 1H), 7.88 (dd, | | | | | | |
| | | J = 7.9, 5.3 Hz, 1H), 7.78 (dd, J = | | | | | | |
| | | 7.8, 5.5 Hz, 2H), 7.74-7.64 (m, | | | | | | |
| | | 1H), 7.49-7.31 (m, 2H), 3.21 (d, | | | | | | |
| | | J = 4.5 Hz, 3H), 2.30 (s, 3H). | | | | | | |
| 507 | TsOH | ¹H NMR (400 MHz, DMSO) δ 9.69 | DMSO | >98 | Method | | | |
| | | (d, J = 1.6 Hz, 1H), 9.17 (d, J = | | | R5 | | | |
| | | 8.1 Hz, 1H), 8.90 (d, J = 3.9 Hz, | | | | | | |
| | | 1H), 8.83 (d, J = 4.3 Hz, 1H), 8.16 | | | | | | |
| | | (d, J = 8.6 Hz, 1H), 8.01-7.87 | | | | | | |
| | | (m, 1H), 7.78 (dd, J = 7.8, 5.5 Hz, | | | | | | |
| | | 2H), 7.75-7.64 (m, 1H), 7.53- | | | | | | |
| | | 7.43 (m, 2H), 7.40 (dd, J = 11.3, | | | | | | |
| | | 4.4 Hz, 2H), 7.11 (d, J = 7.8 Hz, | | | | | | |
| | | 2H), 3.21 (d, J = 4.5 Hz, 3H), 2.29 | | | | | | |
| | | (s, 3H). | | | | | | |
| 508 | 0.5FUM | ¹H NMR (400 MHz, DMSO) δ 13.11 | DMSO | >98 | Method | | | |
| | | (s, 1H), 9.65 (d, J = 1.5 Hz, 1H), | | | R5 | | | |
| | | 8.79 (dt, J = 8.0, 1.9 Hz, 1H), 8.75- | | | | | | |
| | | 8.60 (m, 2H), 8.13 (d, J = 8.5 | | | | | | |
| | | Hz, 1H), 7.78 (dd, J = 7.8, 5.5 Hz, | | | | | | |
| | | 2H), 7.69-7.59 (m, 1H), 7.56 | | | | | | |
| | | (dd, J = 7.9, 4.8 Hz, 1H), 7.40 (t, | | | | | | |
| | | J = 8.9 Hz, 2H), 6.62 (s, 1H), 3.19 | | | | | | |
| | | (d, J = 4.5 Hz, 3H). | | | | | | |
| 509 | | ¹H NMR (400 MHz, DMSO) δ 9.65 | DMSO | >98 | Method | | | |
| | | (s, 1H), 8.79 (d, J = 8.0 Hz, 1H), | | | R5 | | | |
| | | 8.69 (dd, J = 12.4, 4.6 Hz, 2H), | | | | | | |
| | | 8.13 (d, J = 8.7 Hz, 1H), 7.83- | | | | | | |
| | | 7.73 (m, 2H), 7.69-7.61 (m, 1H), | | | | | | |
| | | 7.56 (dd, J = 7.8, 4.8 Hz, 1H), | | | | | | |
| | | 7.40 (t, J = 8.9 Hz, 2H), 3.19 (d, J = | | | | | | |
| | | 4.4 Hz, 3H). | | | | | | |
| 510 | 2HCl | ¹H NMR (400 MHz, DMSO) δ 9.70 | DMSO | >98 | Method | | | |
| | | (s, 1H), 9.29 (d, J = 8.1 Hz, 1H), | | | R5 | | | |
| | | 8.96 (d, J = 4.2 Hz, 2H), 8.21 (d, J = | | | | | | |
| | | 8.7 Hz, 1H), 8.10-7.99 (m, | | | | | | |
| | | 1H), 7.84-7.68 (m, 1H), 7.61 (dt, | | | | | | |
| | | J = 13.1, 7.5 Hz, 3H), 7.35 (dd, J = | | | | | | |
| | | 9.6, 7.9 Hz, 1H), 3.21 (d, J = | | | | | | |
| | | 4.4 Hz, 3H). 1H of 2HCl was not | | | | | | |
| | | observed. | | | | | | |
| 511 | 2HCl | ¹H NMR (400 MHz, DMSO) δ 9.69 | DMSO | >98 | Method | | | |
| | | (s, 1H), 9.37 (6, J = 8.2 Hz, 1H), | | | R5 | | | |
| | | 9.03 (dd, J = 14.5, 4.9 Hz, 2H), | | | | | | |
| | | 8.26 (d, J = 8.6 Hz, 1H), 8.21 (s, | | | | | | |
| | | 1H), 8.13 (dd, J = 8.1, 5.6 Hz, | | | | | | |
| | | 1H), 8.08 (d, J = 7.8 Hz, 1H), 7.98 | | | | | | |
| | | (d, J = 7.8 Hz, 1H), 7.79 (td, J = | | | | | | |
| | | 7.6, 5.2 Hz, 2H), 3.22 (d, J = 4.4 | | | | | | |
| | | Hz, 3H). 1H of 2HCl was not | | | | | | |
| | | observed. | | | | | | |
| 512 | | ¹H NMR (400 MHz, DMSO) δ 9.65 | DMSO | >98 | Method | | | |
| | | (s, 1H), 8.79 (d, J = 8.0 Hz, 1H), | | | R5 | | | |
| | | 8.72 (dd, J = 6.1, 4.6 Hz, 2H), | | | | | | |
| | | 8.17 (d, J = 8.6 Hz, 1H), 8.03 (d, J = | | | | | | |
| | | 8.2 Hz, 2H), 7.93 (d, J = 8.0 Hz, | | | | | | |
| | | 2H), 7.74-7.66 (m, 1H), 7.57 | | | | | | |
| | | (dd, J = 7.9, 4.7 Hz, 1H), 3.19 (d, | | | | | | |
| | | J = 4.4 Hz, 3H). | | | | | | |
| 513 | HCl | ¹H NMR (400 MHz, DMSO) δ 9.68 | DMSO | >98 | Method | | | |
| | | (d, J = 1.6 Hz, 1H), 9.11 (d, J = | | | R5 | | | |
| | | 7.7 Hz, 1H), 8.86 (d, J = 3.7 Hz, | | | | | | |
| | | 2H), 8.19 (d, J = 8.7 Hz, 1H), 7.92- | | | | | | |
| | | 7.81 (m, 1H), 7.59 (dt, J = 7.8, | | | | | | |
| | | 6.6 Hz, 3H), 7.42 (dd, J = 14.9, | | | | | | |
| | | 8.1 Hz, 2H), 3.21 (d, J = 4.4 Hz, | | | | | | |
| | | 3H). | | | | | | |
| | | 1H of 1HCl was not observed. | | | | | | |
| 514 | HCl | ¹H NMR (400 MHz, DMSO) δ 9.69 | DMSO | >98 | Method | | | |
| | | (s, 1H), 9.19 (d, J = 7.9 Hz, 1H), | | | R5 | | | |
| | | 8.91 (d, J = 5.3 Hz, 2H), 8.21 (d, J = | | | | | | |
| | | 8.4 Hz, 1H), 8.03-7.86 (m, | | | | | | |
| | | 1H), 7.71-7.60 (m, 1H), 7.57- | | | | | | |
| | | 7.34 (m, 3H), 3.21 (d, J = 4.4 Hz, | | | | | | |
| | | 3H). 1H of HCl was not observed. | | | | | | |
| 515 | HCl | ¹H NMR (400 MHz, DMSO) δ 9.69 | DMSO | >98 | Method | | | |
| | | (d, J = 1.6 Hz, 1H), 9.14 (d, J = | | | R5 | | | |
| | | 7.8 Hz, 1H), 8.90 (dd, J = 8.6, 4.8 | | | | | | |
| | | Hz, 2H), 8.21 (d, J = 8.6 Hz, 1H), | | | | | | |
| | | 7.98-7.81 (m, 1H), 7.71-7.53 | | | | | | |
| | | (m, 2H), 7.52-7.29 (m, 2H), 3.22 | | | | | | |
| | | (d, J = 4.5 Hz, 3H). 1H of HCl was | | | | | | |
| | | not observed. | | | | | | |
| 516 | HCl | ¹H NMR (400 MHz, DMSO) δ 9.68 | DMSO | >98 | Method | | | |
| | | (d, J = 1.7 Hz, 1H), 9.19 (d, J = | | | R5 | | | |
| | | 8.3 Hz, 1H), 8.91 (d, J = 5.0 Hz, | | | | | | |
| | | 2H), 8.20 (d, J = 8.6 Hz, 1H), 7.99- | | | | | | |
| | | 7.86 (m, 1H), 7.83-7.69 (m, | | | | | | |
| | | 1H), 7.51 (d, J = 6.9 Hz, 2H), 7.41 | | | | | | |
| | | (tt, J = 9.3, 2.3 Hz, 1H), 3.21 (d, J = | | | | | | |
| | | 4.5 Hz, 3H). 1H of HCl was not | | | | | | |
| | | observed. | | | | | | |
| 517 | HCl | ¹H NMR (400 MHz, DMSO) δ 9.69 | DMSO | >98 | Method | | | |
| | | (s, 1H), 9.18 (d, J = 8.4 Hz, 1H), | | | R5 | | | |
| | | 8.96-8.76 (m, 2H), 8.19 (d, J = | | | | | | |
| | | 8.5 Hz, 1H), 7.98-7.91 (m, 1H), | | | | | | |
| | | 7.90-7.80 (m, 1H), 7.78-7.69 | | | | | | |
| | | (m, 1H), 7.69-7.56 (m, 2H), 3.21 | | | | | | |
| | | (d, J = 4.4 Hz, 3H). 1H of HCl was | | | | | | |
| | | not observed. | | | | | | |
| 518 | HCl | ¹H NMR (400 MHz, DMSO) δ 9.69 | DMSO | >98 | Method | | | |
| | | (d, J = 1.6 Hz, 1H), 9.24 (d, J = | | | R5 | | | |
| | | 8.2 Hz, 1H), 8.99-8.91 (m, 1H), | | | | | | |
| | | 8.87 (d, J = 4.7 Hz, 1H), 8.16 (d, J = | | | | | | |
| | | 8.8 Hz, 1H), 8.00 (dd, J = 8.0, | | | | | | |
| | | 5.5 Hz, 1H), 7.80-7.62 (m, 3H), | | | | | | |
| | | 7.12 (d, J = 8.8 Hz, 2H), 3.85 (s, | | | | | | |
| | | 3H), 3.21 (d, J = 4.4 Hz, 3H). | | | | | | |
| | | 1H of HCl was not observed. | | | | | | |
| 519 | 2HCl | ¹H NMR (400 MHz, DMSO) δ 9.68 | DMSO | >98 | Method | | | |
| | | (s, 1H), 9.11 (d, J = 7.7 Hz, 1H), | | | R5 | | | |
| | | 9.02-8.81 (m, 4H), 8.27 (d, J = | | | | | | |
| | | 8.5 Hz, 1H), 8.05 (d, J = 5.0 Hz, | | | | | | |
| | | 2H), 7.94-7.71 (m, 2H), 3.22 (d, | | | | | | |
| | | J = 4.4 Hz, 3H). 1H of 2HCl was | | | | | | |
| | | not observed. | | | | | | |
| 520 | HCl | ¹H NMR (400 MHz, DMSO) δ 9.68 | DMSO | >98 | Method | | | |
| | | (d, J = 1.6 Hz, 1H), 9.09 (d, J = | | | R5 | | | |
| | | 7.8 Hz, 1H), 8.87 (d, J = 5.1 Hz, | | | | | | |
| | | 2H), 8.19 (d, J = 8.6 Hz, 1H), 7.93- | | | | | | |
| | | 7.81 (m, 1H), 7.81-7.66 (m, | | | | | | |
| | | 3H), 3.20 (d, J = 4.4 Hz, 3H). 1H | | | | | | |
| | | of HCl was not observed. | | | | | | |
| 521 | HCl | ¹H NMR (400 MHz, DMSO) δ 9.68 | DMSO | >98 | Method | | | |
| | | (s, 1H), 9.09 (d, J = 8.1 Hz, 1H), | | | R5 | | | |
| | | 8.88 (dd, J = 10.9, 4.4 Hz, 2H), | | | | | | |
| | | 8.21 (d, J = 8.6 Hz, 1H), 7.89- | | | | | | |
| | | 7.80 (m, 1H), 7.67-7.59 (m, 1H), | | | | | | |
| | | 7.58-7.46 (m, 2H), 3.21 (d, J = | | | | | | |
| | | 4.4 Hz, 3H). 1H of HCl was not | | | | | | |
| | | observed. | | | | | | |
| 522 | HCl | ¹H NMR (400 MHz, DMSO) δ 9.68 | DMSO | >98 | Method | | | |
| | | (d, J = 1.5 Hz, 1H), 9.08 (d, J = | | | R5 | | | |
| | | 8.1 Hz, 1H), 8.93-8.77 (m, 2H), | | | | | | |
| | | 8.19 (d, J = 8.5 Hz, 1H), 7.94- | | | | | | |
| | | 7.74 (m, 3H), 7.70-7.50 (m, 1H), | | | | | | |
| | | 3.21 (d, J = 4.5 Hz, 3H). 1H of HCl | | | | | | |
| | | was not observed. | | | | | | |
| 523 | HCl | ¹H NMR (400 MHz, DMSO) δ 9.68 | DMSO | >98 | Method | | | |
| | | (s, 1H), 9.14 (d, J = 8.0 Hz, 1H), | | | R5 | | | |
| | | 8.91 (dd, J = 15.5, 4.6 Hz, 2H), | | | | | | |
| | | 8.22 (d, J = 8.6 Hz, 1H), 7.96- | | | | | | |
| | | 7.82 (m, 1H), 7.81-7.70 (m, 1H), | | | | | | |
| | | 7.70-7.56 (m, 1H), 7.52-7.38 | | | | | | |
| | | (m, 1H), 3.21 (d, J = 4.5 Hz, 3H). | | | | | | |
| | | 1H of HCl was not observed. | | | | | | |
| 524 | | ¹H NMR (400 MHz, DMSO) δ 9.64 | DMSO | >98 | Method | | | |
| | | (d, J = 1.7 Hz, 1H), 8.78 (dd, J = | | | R5 | | | |
| | | 7.9, 1.9 Hz, 1H), 8.71 (dd, J = 4.8, | | | | | | |
| | | 1.6 Hz, 1H), 8.66 (d, J = 4.4 Hz, | | | | | | |
| | | 1H), 8.11 (d, J = 8.8 Hz, 1H), 7.97 | | | | | | |
| | | (d, J = 1.4 Hz, 1H), 7.94-7.82 | | | | | | |
| | | (m, 1H), 7.56 (dd, J = 7.9, 4.8 Hz, | | | | | | |
| | | 1H), 7.17 (t, J = 3.1 Hz, 1H), 6.77 | | | | | | |
| | | (dd, J = 3.4, 1.8 Hz, 1H), 3.17 (d, | | | | | | |
| | | J = 4.4 Hz, 3H). | | | | | | |
| 525 | HCl | ¹H NMR (400 MHz, DMSO) δ 9.69 | DMSO | >98 | Method | | | |
| | | (d, J = 1.6 Hz, 1H), 9.18 (d, J = | | | R5 | | | |
| | | 8.3 Hz, 1H), 8.91 (d, J = 5.2 Hz, | | | | | | |
| | | 1H), 8.86 (s, 1H), 8.17 (d, J = 8.7 | | | | | | |
| | | Hz, 1H), 7.95 (d, J = 5.1 Hz, 1H), | | | | | | |
| | | 7.79-7.61 (m, 1H), 7.52 (d, J = | | | | | | |
| | | 11.8 Hz, 2H), 7.45 (t, J = 7.6 Hz, | | | | | | |
| | | 1H), 7.31 (d, J = 7.4 Hz, 1H), 3.21 | | | | | | |
| | | (d, J = 4.5 Hz, 3H), 2.42 (s, 3H). | | | | | | |
| | | 1H of HCl was not observed. | | | | | | |
| 526 | HCl | ¹H NMR (400 MHz, DMSO) δ 9.69 | DMSO | >98 | Method | | | |
| | | (s, 1H), 9.28 (d, J = 7.8 Hz, 1H), | | | R5 | | | |
| | | 8.93 (dd, J = 17.5, 4.4 Hz, 2H), | | | | | | |
| | | 8.18 (d, J = 8.6 Hz, 1H), 8.10- | | | | | | |
| | | 7.93 (m, 1H), 7.70 (dd, J = 8.5, | | | | | | |
| | | 7.3 Hz, 1H), 7.62 (d, J = 7.1 Hz, | | | | | | |
| | | 2H), 7.38 (d, J = 7.9 Hz, 2H), 3.21 | | | | | | |
| | | (d, J = 4.5 Hz, 3H), 2.40 (s, 3H). | | | | | | |
| | | 1H of HCl was not observed. | | | | | | |
| 527 | | ¹H NMR (400 MHz, DMSO) δ 9.65 | DMSO | >98 | Method | | | |
| | | (d, J = 2.0 Hz, 1H), 8.79 (dt, J = | | | R5 | | | |
| | | 8.0, 1.9 Hz, 1H), 8.70 (dd, J = 4.7, | | | | | | |
| | | 1.6 Hz, 1H), 8.63 (d, J = 4.4 Hz, | | | | | | |
| | | 1H), 8.15 (s, 1H), 8.09 (d, J = 8.7 | | | | | | |
| | | Hz, 1H), 7.92-7.82 (m, 1H), 7.77 | | | | | | |
| | | (dd, J = 5.0, 2.9 Hz, 1H), 7.71 (d, | | | | | | |
| | | J = 5.0 Hz, 1H), 7.56 (dd, J = 7.9, | | | | | | |
| | | 4.8 Hz, 1H), 3.18 (d, J = 4.4 Hz, | | | | | | |
| | | 3H). | | | | | | |
| 528 | 2HCl | ¹H NMR (400 MHz, DMSO) δ 9.70 | DMSO | >98 | Method | | | |
| | | (s, 1H), 9.25 (d, J = 7.8 Hz, 1H), | | | R5 | | | |
| | | 8.94 (d, J = 5.3 Hz, 2H), 8.23 (d, J = | | | | | | |
| | | 8.7 Hz, 1H), 8.00 (dd, J = 13.5, | | | | | | |
| | | 5.9 Hz, 1H), 7.99-7.87 (m, 4H), | | | | | | |
| | | 7.82-7.72 (m, 1H), 3.22 (d, J = | | | | | | |
| | | 4.5 Hz, 3H). 1H of 2HCl was not | | | | | | |
| | | observed. | | | | | | |
| 529 | HCl | ¹H NMR (400 MHz, DMSO) δ 9.70 | DMSO | >98 | Method | | | |
| | | (d, J = 1.7 Hz, 1H), 9.27 (d, J = | | | R5 | | | |
| | | 7.9 Hz, 1H), 8.99-8.83 (m, 2H), | | | | | | |
| | | 8.22 (dd, J = 16.3, 6.6 Hz, 3H), | | | | | | |
| | | 8.02 (dd, J = 7.9, 5.4 Hz, 1H), | | | | | | |
| | | 7.89 (d, J = 5.4 Hz, 1H), 7.84- | | | | | | |
| | | 7.77 (m, 1H), 7.71 (d, J = 8.4 Hz, | | | | | | |
| | | 1H), 7.58 (d, J = 5.5 Hz, 1H), 3.22 | | | | | | |
| | | (d, J = 4.5 Hz, 3H). 1H of HCl was | | | | | | |
| | | not observed. | | | | | | |
| 530 | | ¹H NMR (400 MHz, DMSO) δ 9.64 | DMSO | >98 | Method | | | |
| | | (d, J = 1.5 Hz, 1H), 8.79 (dt, J = | | | R5 | | | |
| | | 8.0, 1.9 Hz, 1H), 8.71 (dd, J = 4.7, | | | | | | |
| | | 1.7 Hz, 1H), 8.62 (d, J = 4.5 Hz, | | | | | | |
| | | 1H), 8.39 (s, 1H), 8.08 (d, J = 8.5 | | | | | | |
| | | Hz, 1H), 7.94-7.81 (m, 2H), 7.56 | | | | | | |
| | | (dd, J = 7.9, 4.8 Hz, 1H), 7.20 (s, | | | | | | |
| | | 1H), 3.18 (d, J = 4.5 Hz, 3H). | | | | | | |
| 531 | HCl | ¹H NMR (400 MHz, DMSO) δ 9.69 | DMSO | >98 | Method | | | |
| | | (d, J = 1.6 Hz, 1H), 9.15 (d, J = | | | R5 | | | |
| | | 8.0 Hz, 1H), 8.88 (d, J = 5.1 Hz, | | | | | | |
| | | 1H), 8.80 (s, 1H), 8.15 (d, J = 8.8 | | | | | | |
| | | Hz, 1H), 7.91 (s, 2H), 7.79-7.71 | | | | | | |
| | | (m, 1H), 7.61 (d, J = 8.6 Hz, 1H), | | | | | | |
| | | 7.51 (d, J = 8.6 Hz, 1H), 7.43 (d, J = | | | | | | |
| | | 3.1 Hz, 1H), 6.55 (d, J = 2.4 Hz, | | | | | | |
| | | 1H), 3.86 (s, 3H), 3.21 (d, J = 4.5 | | | | | | |
| | | Hz, 3H). 1H was not observed. | | | | | | |
| 532 | HCl | ¹H NMR (400 MHz, DMSO) δ 9.70 | DMSO | >98 | Method | | | |
| | | (d, J = 1.5 Hz, 1H), 9.32 (d, J = | | | R5 | | | |
| | | 8.2 Hz, 1H), 8.96 (dd, J = 12.1, | | | | | | |
| | | 4.9 Hz, 2H), 8.26-8.15 (m, 2H), | | | | | | |
| | | 8.15-8.02 (m, 2H), 7.79 (dt, J = | | | | | | |
| | | 13.6, 8.7 Hz, 3H), 4.12 (s, 3H), | | | | | | |
| | | 3.22 (d, J = 4.4 Hz, 3H). 1H of HCl | | | | | | |
| | | was not observed. | | | | | | |
| 533 | 2HCl | ¹H NMR (400 MHz, DMSO) δ 9.64 | DMSO | >98 | Method | | | |
| | | (s, 1H), 8.79 (d, J = 8.1 Hz, 1H), | | | R5 | | | |
| | | 8.70 (t, J = 7.8 Hz, 1H), 8.65 (d, J = | | | | | | |
| | | 4.3 Hz, 1H), 8.09 (d, J = 8.7 Hz, | | | | | | |
| | | 1H), 7.99-7.93 (m, 1H), 7.88 (d, | | | | | | |
| | | J = 3.6 Hz, 1H), 7.83 (d, J = 5.1 | | | | | | |
| | | Hz, 1H), 7.56 (dd, J = 7.8, 4.8 Hz, | | | | | | |
| | | 1H), 7.29 (s, 1H), 3.18 (d, J = 4.3 | | | | | | |
| | | Hz, 3H). | | | | | | |
| 534 | 2HCl | ¹H NMR (400 MHz, DMSO) δ 9.69 | DMSO | >98 | Method | | | |
| | | (d, J = 1.6 Hz, 1H), 9.13 (d, J = | | | R5 | | | |
| | | 8.1 Hz, 1H), 8.88 (t, J = 4.6 Hz, | | | | | | |
| | | 2H), 8.33-8.13 (m, 2H), 8.14- | | | | | | |
| | | 8.02 (m, 2H), 7.99 (dd, J = 5.5, | | | | | | |
| | | 3.6 Hz, 1H), 7.90 (dd, J = 8.0, 5.2 | | | | | | |
| | | Hz, 1H), 7.60-7.31 (m, 2H), 3.21 | | | | | | |
| | | (d, J = 4.5 Hz, 3H). 1H of HCl was | | | | | | |
| | | not observed | | | | | | |
| 535 | HCl | ¹H NMR (400 MHz, DMSO) δ 13.28 | DMSO | >98 | Method | | | |
| | | (s, 1H), 9.69 (d, J = 1.5 Hz, 1H), | | | R5 | | | |
| | | 9.16 (d, J = 8.2 Hz, 1H), 8.89 (dd, | | | | | | |
| | | J = 5.2, 1.5 Hz, 1H), 8.84 (d, J = | | | | | | |
| | | 4.2 Hz, 1H), 8.20 (s, 1H), 8.18 (d, | | | | | | |
| | | J = 8.6 Hz, 1H), 8.13 (s, 1H), 7.97- | | | | | | |
| | | 7.87 (m, 1H), 7.81-7.73 (m, | | | | | | |
| | | 1H), 7.71 (s, 2H), 3.21 (d, J = 4.5 | | | | | | |
| | | Hz, 3H). 1H of HCl was not | | | | | | |
| | | observed. | | | | | | |
| 536 | HCl | ¹H NMR (400 MHz, DMSO) δ 9.69 | DMSO | >98 | Method | | | |
| | | (d, J = 1.7 Hz, 1H), 9.13 (s, 1H), | | | R5 | | | |
| | | 8.89 (s, 2H), 8.23 (d, J = 8.8 Hz, | | | | | | |
| | | 1H), 8.12 (dd, J = 8.7, 6.8 Hz, | | | | | | |
| | | 1H), 7.90 (s, 1H), 7.81 (d, J = 7.5 | | | | | | |
| | | Hz, 1H), 7.72 (d, J = 8.4 Hz, 1H), | | | | | | |
| | | 7.65 (d, J = 2.5 Hz, 1H), 7.51- | | | | | | |
| | | 7.39 (m, 1H), 7.38-7.29 (m, 1H), | | | | | | |
| | | 3.20 (d, J = 4.5 Hz, 3H). 1H of HCl | | | | | | |
| | | was not observed. | | | | | | |
| 537 | 2HCl | ¹H NMR (400 MHz, DMSO) δ 9.71 | DMSO | >98 | Method | | | |
| | | (d, J = 1.7 Hz, 1H), 9.23 (d, J = | | | R5 | | | |
| | | 7.5 Hz, 1H), 9.02-8.82 (m, 2H), | | | | | | |
| | | 8.24 (d, J = 8.6 Hz, 1H), 8.18- | | | | | | |
| | | 8.12 (m, 1H), 8.11 (s, 1H), 8.02- | | | | | | |
| | | 7.93 (m, 1H), 7.79-7.67 (m, 2H), | | | | | | |
| | | 7.55-7.43 (m, 2H), 3.23 (d, J = | | | | | | |
| | | 4.5 Hz, 3H). 1H of 2HCl was not | | | | | | |
| | | observed. | | | | | | |
| 538 | 2HCl | ¹H NMR (400 MHz, DMSO) δ 9.71 | DMSO | >98 | Method | | | |
| | | (d, J = 1.6 Hz, 1H), 9.25 (d, J = | | | R5 | | | |
| | | 7.8 Hz, 1H), 8.91 (t, J = 5.2 Hz, | | | | | | |
| | | 2H), 8.24 (d, J = 8.5 Hz, 1H), 8.17 | | | | | | |
| | | (d, J = 7.2 Hz, 1H), 8.05-7.96 | | | | | | |
| | | (m, 1H), 7.86 (d, J = 5.6 Hz, 1H), | | | | | | |
| | | 7.73-7.63 (m, 1H), 7.54 (dt, J = | | | | | | |
| | | 7.2, 6.7 Hz, 2H), 7.22 (dd, J = 5.4, | | | | | | |
| | | 2.1 Hz, 1H), 3.23 (d, J = 4.4 Hz, | | | | | | |
| | | 3H). 1H of 2HCl was not observed. | | | | | | |
| 539 | HCl | ¹H NMR (400 MHz, DMSO) δ 9.70 | DMSO | >98 | Method | | | |
| | | (s, 1H), 9.23 (d, J = 7.8 Hz, 1H), | | | R5 | | | |
| | | 8.91 (dd, J = 11.8, 4.9 Hz, 2H), | | | | | | |
| | | 8.39 (s, 1H), 8.21 (d, 7 = 8.8 Hz, | | | | | | |
| | | 1H), 8.06 (d, J = 8.3 Hz, 1H), 7.96 | | | | | | |
| | | (dd, J = 20.5, 15.2 Hz, 1H), 7.90 | | | | | | |
| | | (d, J = 5.4 Hz, 1H), 7.80 (dd, J = | | | | | | |
| | | 19.1, 11.5 Hz, 1H), 7.74 (d, J = | | | | | | |
| | | 8.3 Hz, 1H), 7.56 (d, J = 5.4 Hz, | | | | | | |
| | | 1H), 3.22 (d, J = 4.3 Hz, 3H). 1H | | | | | | |
| | | of HCl was not observed. | | | | | | |
| 540 | HCl | ¹H NMR (400 MHz, DMSO) δ 9.71 | DMSO | >98 | Method | | | |
| | | (s, 1H), 9.29 (d, J = 8.0 Hz, 1H), | | | R5 | | | |
| | | 8.98 (dd, J = 16.2, 5.0 Hz, 2H), | | | | | | |
| | | 8.26 (d, J = 8.5 Hz, 1H), 8.04 (d, J = | | | | | | |
| | | 7.7 Hz, 2H), 7.84 (d, J = 5.4 Hz, | | | | | | |
| | | 1H), 7.81-7.74 (m, 1H), 7.67- | | | | | | |
| | | 7.47 (m, 3H), 3.24 (d, J = 4.4 Hz, | | | | | | |
| | | 3H). 1H of HCl was not observed. | | | | | | |
| 541 | HCl | ¹H NMR (400 MHz, DMSO) δ 9.68 | DMSO | >98 | Method | | | |
| | | (d, J = 1.7 Hz, 1H), 9.34 (d, J = | | | R5 | | | |
| | | 8.2 Hz, 1H), 8.99 (d, J = 4.3 Hz, | | | | | | |
| | | 2H), 8.21 (d, J = 8.6 Hz, 1H), 8.11 | | | | | | |
| | | (dd, J = 8.0, 5.6 Hz, 1H), 7.68 (dt, | | | | | | |
| | | J = 33.2, 12.4 Hz, 2H), 7.59 (s, | | | | | | |
| | | 1H), 7.44-7.22 (m, 1H), 3.21 (d, | | | | | | |
| | | J = 4.5 Hz, 3H), 2.34 (d, J = 1.5 | | | | | | |
| | | Hz, 3H). 1H of HCl was not | | | | | | |
| | | observed | | | | | | |
| 542 | 2HCl | ¹H NMR (400 MHz, DMSO) δ 9.68 | DMSO | >98 | Method | | | |
| | | (s, 1H), 9.18 (d, J = 7.3 Hz, 1H), | | | R5 | | | |
| | | 8.90 (d, J = 5.1 Hz, 2H), 8.16 (d, J = | | | | | | |
| | | 8.7 Hz, 1H), 7.94 (s, 1H), 7.51- | | | | | | |
| | | 7.42 (m, 1H), 7.42-7.32 (m, 1H), | | | | | | |
| | | 7.28 (d, J = 7.5 Hz, 1H), 7.19 (t, J = | | | | | | |
| | | 8.7 Hz, 1H), 3.21 (d, J = 4.5 Hz, | | | | | | |
| | | 3H), 2.20 (s, 3H). 1H of 2HCl was | | | | | | |
| | | not observed. | | | | | | |
| 543 | 2HCl | ¹H NMR (400 MHz, DMSO) δ 9.69 | DMSO | >98 | Method | | | |
| | | (d, J = 1.6 Hz, 1H), 9.30 (d, J = | | | R5 | | | |
| | | 8.0 Hz, 1H), 8.96 (d, J = 5.4 Hz, | | | | | | |
| | | 1H), 8.92 (d, J = 4.7 Hz, 1H), 8.14 | | | | | | |
| | | (d, J = 8.5 Hz, 1H), 8.06 (dd, J = | | | | | | |
| | | 8.0, 5.5 Hz, 1H), 7.51 (dd, J = 8.4, | | | | | | |
| | | 6.8 Hz, 1H), 7.41 (dd, J = 8.4, 6.9 | | | | | | |
| | | Hz, 1H), 7.13 (dd, J = 11.5, 2.4 | | | | | | |
| | | Hz, 1H), 6.95 (td, J = 8.4, 2.4 Hz, | | | | | | |
| | | 1H), 3.78 (s, 3H), 3.21 (d, J = 4.4 | | | | | | |
| | | Hz, 3H). 1H of 2HCl was not | | | | | | |
| | | observed. | | | | | | |
| 544 | 2HCl | ¹H NMR (400 MHz, DMSO) δ 9.68 | DMSO | >98 | Method | | | |
| | | (s, 1H), 9.18 (d, J = 8.3 Hz, 1H), | | | R5 | | | |
| | | 8.90 (d, J = 4.1 Hz, 1H), 8.86 (s, | | | | | | |
| | | 1H), 8.16 (d, J = 8.6 Hz, 1H), 7.94 | | | | | | |
| | | (s, 1H), 7.73 (t, J = 7.9 Hz, 1H), | | | | | | |
| | | 7.64 (d, J = 13.1 Hz, 1H), 7.56 (d, | | | | | | |
| | | J = 8.7 Hz, 1H), 7.35 (t, J = 8.8 | | | | | | |
| | | Hz, 1H), 3.93 (s, 3H), 3.20 (d, J = | | | | | | |
| | | 4.3 Hz, 3H). 1H of 2HCl was not | | | | | | |
| | | observed. | | | | | | |
| 545 | 2HCl | 1H NMR (400 MHz, DMSO) δ 9.69 | DMSO | >98 | Method | | | |
| | | (d, J = 1.7 Hz, 1H), 9.26 (d, J = | | | R5 | | | |
| | | 8.4 Hz, 1H), 9.00-8.85 (m, 2H), | | | | | | |
| | | 8.19 (d, J = 8.6 Hz, 1H), 8.09- | | | | | | |
| | | 7.96 (m, 1H), 7.85-7.68 (m, 1H), | | | | | | |
| | | 7.61-7.49 (m, 1H), 7.52-7.40 | | | | | | |
| | | (m, 2H), 3.21 (d, J = 4.5 Hz, 3H), | | | | | | |
| | | 2.33 (s, 3H). 1H of 2HCl was not | | | | | | |
| | | observed. | | | | | | |
| 546 | 2HCl | ¹H NMR (400 MHz, DMSO) δ 9.69 | DMSO | >98 | Method | | | |
| | | (d, J = 1.8 Hz, 1H), 9.36 (d, J = | | | R5 | | | |
| | | 8.1 Hz, 1H), 9.05-8.90 (m, 2H), | | | | | | |
| | | 8.22 (d, J = 8.6 Hz, 1H), 8.11 (dd, | | | | | | |
| | | J = 8.0, 5.6 Hz, 1H), 7.65-7.57 | | | | | | |
| | | (m, 1H), 7.48 (t, J = 7.8 Hz, 1H), | | | | | | |
| | | 7.24 (dd, J = 13.4, 9.6 Hz, 2H), | | | | | | |
| | | 3.22 (d, J = 4.5 Hz, 3H), 2.42 (s, | | | | | | |
| | | 3H). 1H of 2HCl was not observed. | | | | | | |
| 547 | 2HCl | ¹H NMR (400 MHz, DMSO) δ 9.69 | DMSO | >98 | Method | | | |
| | | (d, J = 1.7 Hz, 1H), 9.26 (d, J = | | | R5 | | | |
| | | 7.9 Hz, 1H), 8.95 (d, J = 3.9 Hz, | | | | | | |
| | | 2H), 8.19 (d, J = 8.7 Hz, 1H), 8.02 | | | | | | |
| | | (dd, J = 8.0, 5.3 Hz, 1H), 7.89- | | | | | | |
| | | 7.67 (m, 1H), 7.23-7.08 (m, 2H), | | | | | | |
| | | 6.99 (dt, J = 11.0, 2.2 Hz, 1H), | | | | | | |
| | | 3.87 (s, 3H), 3.21 (d, J = 4.5 Hz, | | | | | | |
| | | 3H).1H of 2HCl was not observed. | | | | | | |
| 548 | 2HCl | ¹H NMR (400 MHz, DMSO) δ 9.69 | DMSO | >98 | Method | | | |
| | | (d, J = 1.6 Hz, 1H), 9.19 (d, J = | | | R5 | | | |
| | | 7.3 Hz, 1H), 8.90 (d, J = 5.1 Hz, | | | | | | |
| | | 1H), 8.85 (s, 1H), 8.13 (d, J = 8.6 | | | | | | |
| | | Hz, 1H), 7.95 (s, 1H), 7.65-7.48 | | | | | | |
| | | (m, 1H), 7.43-7.24 (m, 2H), 7.21 | | | | | | |
| | | (dd, J = 9.1, 4.6 Hz, 1H), 3.77 (s, | | | | | | |
| | | 3H), 3.21 (d, J = 4.5 Hz, 3H). 1H | | | | | | |
| | | of 2HCl was not observed. | | | | | | |
| 549 | HCl | ¹H NMR (400 MHz, DMSO) δ 9.70 | DMSO | >98 | Method | | | |
| | | (d, J = 1.6 Hz, 1H), 9.26 (d, J = | | | R5 | | | |
| | | 7.8 Hz, 1H), 8.95 (d, J = 5.4 Hz, | | | | | | |
| | | 2H), 8.20 (d, J = 8.6 Hz, 1H), 8.09- | | | | | | |
| | | 7.91 (m, 1H), 7.73-7.53 (m, | | | | | | |
| | | 1H), 7.41-7.22 (m, 2H), 7.20- | | | | | | |
| | | 7.03 (m, 1H), 3.92 (s, 3H), 3.22 | | | | | | |
| | | (d, J = 4.5 Hz, 3H). 1H of HCl was | | | | | | |
| | | not observed. | | | | | | |
| 550 | 2HCl | ¹H NMR (400 MHz, DMSO) δ 9.65 | DMSO | >98 | Method | | | |
| | | (s, 1H), 9.20 (brs, 1H), 9.09 (s, | | | R5 | | | |
| | | 1H), 8.91 (d, J = 5.1 Hz, 1H), 8.36 | | | | | | |
| | | (d, J = 11.2 Hz, 1H), 8.10 (d, J = | | | | | | |
| | | 7.0 Hz, 1H), 7.91 (s, 1H), 7.60 (dt, | | | | | | |
| | | J = 19.4, 7.6 Hz, 3H), 7.38 (t, J = | | | | | | |
| | | 7.8 Hz, 1H), 3.24 (d, J = 4.4 Hz, | | | | | | |
| | | 3H). 1H of 2HCl was not observed. | | | | | | |
| 551 | 2HCl | ¹H NMR (400 MHz, DMSO) δ 9.66 | DMSO | >98 | Method | | | |
| | | (s, 1H), 9.26-9.15 (m, 1H), 9.10 | | | R5 | | | |
| | | (s, 1H), 8.92 (s, 1H), 8.37 (d, J = | | | | | | |
| | | 10.7 Hz, 1H), 8.02 (d, J = 6.3 Hz, | | | | | | |
| | | 1H), 7.92 (s, 1H), 7.71 (dd, J = | | | | | | |
| | | 15.4, 8.5 Hz, 1H), 7.50 (t, J = 9.0 | | | | | | |
| | | Hz, 1H), 7.32 (t, J = 8.5 Hz, 1H), | | | | | | |
| | | 3.24 (d, J = 4.3 Hz, 3H). 1H of | | | | | | |
| | | 2HCl was not observed. | | | | | | |
| 552 | 2HCl | ¹H NMR (400 MHz, DMSO) δ 9.66 | DMSO | >98 | Method | | | |
| | | (s, 1H), 9.27 (brs, 1H), 9.12 (d, J = | | | R5 | | | |
| | | 7.5 Hz, 1H), 8.92 (d, J = 5.0 Hz, | | | | | | |
| | | 1H), 8.38 (d, J = 10.4 Hz, 1H), | | | | | | |
| | | 8.04 (d, J = 7.0 Hz, 1H), 7.97- | | | | | | |
| | | 7.87 (m, 1H), 7.70-7.53 (m, 2H), | | | | | | |
| | | 7.43 (dd, J = 14.6, 7.6 Hz, 2H), | | | | | | |
| | | 3.25 (d, J = 4.3 Hz, 3H). 1H of | | | | | | |
| | | 2HCl was not observed | | | | | | |
| 553 | 2HCl | ¹H NMR (400 MHz, DMSO) δ 9.64 | DMSO | >98 | Method | | | |
| | | (s, 1H), 9.27-9.08 (m, 1H), 9.04 | | | R5 | | | |
| | | (m, 1H), 8.89 (d, J = 5.0 Hz, 1H), | | | | | | |
| | | 8.33 (d, J = 11.4 Hz, 1H), 8.04 (d, | | | | | | |
| | | J = 7.8 Hz, 1H), 7.87 (m, 1H), | | | | | | |
| | | 7.82-7.70 (m, 2H), 7.41 (t, J = | | | | | | |
| | | 8.9 Hz, 2H), 3.24 (d, J = 4.4 Hz, | | | | | | |
| | | 3H). 1H of 2HCl was not observed. | | | | | | |
| 554 | 2HCl | ¹H NMR (400 MHz, DMSO) δ 9.65 | DMSO | >98 | Method | | | |
| | | (s, 1H), 9.04 (brd, 1H), 9.01- | | | R5 | | | |
| | | 8.92 (m, 1H), 8.87 (s, 1H), 8.33 | | | | | | |
| | | (d, J = 10.7 Hz, 1H), 8.01 (d, J = | | | | | | |
| | | 7.0 Hz, 1H), 7.86 (m, 1H), 7.58 | | | | | | |
| | | (m, 1H), 7.46 (m, 2H), 3.23 (d, J = | | | | | | |
| | | 4.3 Hz, 3H). 1H of 2HCl was not | | | | | | |
| | | observed. | | | | | | |
| 555 | 2HCl | ¹H NMR (400 MHz, DMSO) δ 9.67 | DMSO | >98 | Method | | | |
| | | (d, J = 1.6 Hz, 1H), 9.36 (brs, 1H), | | | R5 | | | |
| | | 9.20 (d, J = 8.0 Hz, 1H), 8.96 (dd, | | | | | | |
| | | J = 5.3, 1.4 Hz, 1H), 8.43 (d, J = | | | | | | |
| | | 11.4 Hz, 1H), 8.19 (d, J = 6.8 Hz, | | | | | | |
| | | 1H), 8.06-7.94 (m, 1H), 7.57- | | | | | | |
| | | 7.38 (m, 3H), 3.24 (d, J = 4.5 Hz, | | | | | | |
| | | 3H). 1H of 2HCl was not observed. | | | | | | |
| 556 | | ¹H NMR (400 MHz, DMSO) δ 9.64 | DMSO | >98 | Method | | | |
| | | (s, 1H), 8.77 (d, J = 7.9 Hz, 1H), | | | R5 | | | |
| | | 8.70 (brs, 1H), 8.53 (s, 1H), 8.24 | | | | | | |
| | | (d, J = 11.6 Hz, 1H), 8.21 (s, 1H), | | | | | | |
| | | 8.10-7.93 (m, 3H), 7.76 (t, J = | | | | | | |
| | | 7.8 Hz, 1H), 7.56 (dd, J = 7.9, 4.6 | | | | | | |
| | | Hz, 1H), 3.19 (d, J = 4.4 Hz, 3H). | | | | | | |
| 557 | 3HCl | ¹H NMR (400 MHz, DMSO) δ 9.67 | DMSO | >98 | Method | | | |
| | | (s, 1H), 9.23 (brs, 1H), 9.14 (d, J = | | | R5 | | | |
| | | 7.7 Hz, 1H), 8.92 (d, J = 5.0 Hz, | | | | | | |
| | | 1H), 8.41 (d, J = 10.9 Hz, 1H), | | | | | | |
| | | 8.08 (d, J = 6.4 Hz, 1H), 7.98- | | | | | | |
| | | 7.87 (m, 1H), 7.64 (dd, J = 16.4, | | | | | | |
| | | 8.4 Hz, 1H), 7.51-7.33 (m, 2H), | | | | | | |
| | | 3.24 (d, J = 4.4 Hz, 3H). 1H of | | | | | | |
| | | 3HCl was not observed. | | | | | | |
| 558 | 2HCl | ¹H NMR (400 MHz, DMSO) δ 9.65 | DMSO | >98 | Method | | | |
| | | (d, J = 1.6 Hz, 1H), 9.15 (brs, 1H), | | | R5 | | | |
| | | 9.11 (d, J = 6.8 Hz, 1H), 8.92 (dd, | | | | | | |
| | | J = 5.2, 1.4 Hz, 1H), 8.36 (d, J = | | | | | | |
| | | 11.5 Hz, 1H), 8.09 (d, J = 7.4 Hz, | | | | | | |
| | | 1H), 7.97-7.89 (m, 1H), 7.89- | | | | | | |
| | | 7.79 (m, 1H), 7.71-7.50 (m, 2H), | | | | | | |
| | | 3.24 (d, J = 4.5 Hz, 3H). 1H of | | | | | | |
| | | 2HCl was not observed. | | | | | | |
| 559 | 2HCl | ¹H NMR (400 MHz, DMSO) δ 9.66 | DMSO | >98 | Method | | | |
| | | (d, J = 1.6 Hz, 1H), 9.28 (brs, 1H), | | | R5 | | | |
| | | 9.14 (d, J = 8.0 Hz, 1H), 9.01- | | | | | | |
| | | 8.85 (m, 1H), 8.41 (d, J = 11.5 | | | | | | |
| | | Hz, 1H), 8.15 (d, J = 7.1 Hz, 1H), | | | | | | |
| | | 8.07-8.01 (m, 2H), 7.93 (m, 3H), | | | | | | |
| | | 3.24 (d, J = 4.5 Hz, 3H). 1H of | | | | | | |
| | | 2HCl was not observed. | | | | | | |
| 560 | 2HCl | ¹H NMR (400 MHz, DMSO) δ 9.64 | DMSO | >98 | Method | | | |
| | | (d, J = 1.7 Hz, 1H), 9.54-9.22 | | | R5 | | | |
| | | (m, 1H), 9.06 (s, 1H), 8.92 (dd, J = | | | | | | |
| | | 5.1, 1.4 Hz, 1H), 8.37 (d, J = | | | | | | |
| | | 11.2 Hz, 1H), 8.09 (s, 1H), 7.91 | | | | | | |
| | | (d, J = 5.3 Hz, 1H), 7.67 (dd, J = | | | | | | |
| | | 8.7, 1.7 Hz, 2H), 7.14 (d, J = 8.9 | | | | | | |
| | | Hz, 2H), 3.85 (s, 3H), 3.26 (d, J = | | | | | | |
| | | 4.4 Hz, 3H). 1H of 2HCl was not | | | | | | |
| | | observed. | | | | | | |
| 561 | 2HCl | ¹H NMR (400 MHz, DMSO) δ 9.65 | DMSO | >98 | Method | | | |
| | | (d, J = 1.6 Hz, 1H), 9.43-9.14 | | | R5 | | | |
| | | (m, 1H), 9.07 (s, 1H), 8.91 (d, J = | | | | | | |
| | | 5.0 Hz, 1H), 8.36 (d, J = 12.0 Hz, | | | | | | |
| | | 1H), 8.08 (s, 1H), 7.90 (s, 1H), | | | | | | |
| | | 7.49 (t, J = 8.0 Hz, 1H), 7.36- | | | | | | |
| | | 7.18 (m, 2H), 7.10 (dd, J = 8.0, | | | | | | |
| | | 2.2 Hz, 1H), 3.85 (s, 3H), 3.25 (d, | | | | | | |
| | | J = 4.5 Hz, 3H). 1H of 2HCl was | | | | | | |
| | | not observed. | | | | | | |
| 562 | 2HCl | ¹H NMR (400 MHz, DMSO) δ 9.65 | DMSO | >98 | Method | | | |
| | | (d, J = 1.8 Hz, 1H), 9.11 (s, 1H), | | | R5 | | | |
| | | 8.95 (dd, J = 5.2, 1.5 Hz, 1H), | | | | | | |
| | | 8.39 (d, J = 10.1 Hz, 1H), 8.06 (s, | | | | | | |
| | | 1H), 7.93 (s, 1H), 7.60-7.47 (m, | | | | | | |
| | | 1H), 7.38 (dd, J = 7.5, 1.6 Hz, | | | | | | |
| | | 1H), 7.22 (d, J = 8.0 Hz, 1H), 7.13 | | | | | | |
| | | (dd, J = 7.9, 7.1 Hz, 1H), 3.80 (s, | | | | | | |
| | | 3H), 3.28 (d, J = 4.5 Hz, 3H). 1H | | | | | | |
| | | of 2HCl and NH— were not | | | | | | |
| | | observed. | | | | | | |
| 563 | 2HCl | ¹H NMR (400 MHz, DMSO) δ 9.69 | DMSO | >98 | Method | | | |
| | | (d, J = 1.7 Hz, 1H), 9.24 (d, J = | | | R3 | | | |
| | | 8.4 Hz, 1H), 9.01 (d, J = 4.1 Hz, | | | | | | |
| | | 1H), 8.93 (d, J = 4.2 Hz, 1H), 8.56 | | | | | | |
| | | (s, 1H), 8.19 (dd, J = 12.0, 1.6 Hz, | | | | | | |
| | | 1H), 8.05-7.90 (m, 1H), 7.83- | | | | | | |
| | | 7.72 (m, 2H), 7.60 (td, J = 8.2, | | | | | | |
| | | 6.4 Hz, 1H), 7.36-7.24 (m, 1H), | | | | | | |
| | | 3.23 (d, J = 4.5 Hz, 3H). 1H of | | | | | | |
| | | 2HCl was not observed. | | | | | | |
| 564 | HCl | ¹H NMR (400 MHz, DMSO) δ 9.68 | DMSO | >98 | Method | | | |
| | | (d, J = 1.7 Hz, 1H), 9.21 (d, J = | | | R3 | | | |
| | | 8.0 Hz, 1H), 8.98 (d, J = 4.4 Hz, | | | | | | |
| | | 1H), 8.92 (dd, J = 5.3, 1.4 Hz, | | | | | | |
| | | 1H), 8.49 (s, 1H), 8.12 (dd, J = | | | | | | |
| | | 12.0, 1.7 Hz, 1H), 8.04-7.89 (m, | | | | | | |
| | | 3H), 7.40 (t, J = 8.9 Hz, 2H), 3.22 | | | | | | |
| | | (d, J = 4.5 Hz, 3H). 1H of 2HCl | | | | | | |
| | | was not observed. | | | | | | |
| 565 | HCl | ¹H NMR (400 MHz, DMSO) δ 9.69 | DMSO | >98 | Method | | | |
| | | (d, J = 1.5 Hz, 1H), 9.14 (d, J = | | | R3 | | | |
| | | 7.9 Hz, 1H), 8.98-8.82 (m, 2H), | | | | | | |
| | | 8.35 (s, 1H), 7.91 (dd, J = 12.4, | | | | | | |
| | | 8.2 Hz, 2H), 7.71 (td, J = 7.9, 1.6 | | | | | | |
| | | Hz, 1H), 7.62-7.46 (m, 1H), 7.46- | | | | | | |
| | | 7.34 (m, 2H), 3.20 (d, J = 4.5 | | | | | | |
| | | Hz, 3H). 1H of HCl was not | | | | | | |
| | | observed. | | | | | | |
| 566 | 2HCl | ¹H NMR (400 MHz, DMSO) δ 9.69 | DMSO | >98 | Method | | | |
| | | (d, J = 1.8 Hz, 1H), 9.33 (d, J = | | | R3 | | | |
| | | 8.1 Hz, 1H), 8.98 (dd, J = 5.5, 1.3 | | | | | | |
| | | Hz, 1H), 8.92 (d, J = 4.4 Hz, 1H), | | | | | | |
| | | 8.21 (s, 1H), 8.09 (dd, J = 8.0, 5.5 | | | | | | |
| | | Hz, 1H), 7.88 (dd, J = 11.9, 1.5 | | | | | | |
| | | Hz, 1H), 7.54-7.36 (m, 2H), 7.20 | | | | | | |
| | | (d, J = 7.8 Hz, 1H), 7.12 (td, J = | | | | | | |
| | | 7.5, 0.9 Hz, 1H), 3.83 (s, 3H), | | | | | | |
| | | 3.20 (d, J = 4.5 Hz, 3H). 1H of | | | | | | |
| | | 2HCl was not observed. | | | | | | |
| 567 | | ¹H NMR (400 MHz, DMSO) δ 9.66 | DMSO | >98 | Method | | | |
| | | (s, 1H), 8.87-8.76 (m, 2H), 8.72 | | | R3 | | | |
| | | (dd, J = 4.7, 1.6 Hz, 1H), 8.33 (s, | | | | | | |
| | | 1H), 8.05 (d, J = 7.8 Hz, 1H), 7.96 | | | | | | |
| | | (dd, J = 11.3, 1.6 Hz, 1H), 7.89 (t, | | | | | | |
| | | J = 7.7 Hz, 1H), 7.78 (d, J = 7.5 | | | | | | |
| | | Hz, 1H), 7.67 (t, J = 7.6 Hz, 1H), | | | | | | |
| | | 7.58 (dd, J = 7.9, 4.7 Hz, 1H), | | | | | | |
| | | 3.19 (d, J = 4.4 Hz, 3H). | | | | | | |
| 568 | 2HCl | ¹H NMR (400 MHz, DMSO) δ 9.68 | DMSO | >98 | Method | | | |
| | | (d, J = 1.7 Hz, 1H), 9.23 (d, J = | | | R3 | | | |
| | | 8.1 Hz, 1H), 9.07-8.94 (m, 1H), | | | | | | |
| | | 8.93 (d, J = 3.9 Hz, 1H), 8.53 (s, | | | | | | |
| | | 1H), 8.17 (dd, J = 12.0, 1.7 Hz, | | | | | | |
| | | 1H), 8.11-7.87 (m, 2H), 7.79 | | | | | | |
| | | (brs, 1H), 7.72-7.54 (m, 1H), | | | | | | |
| | | 3.23 (d, J = 4.5 Hz, 3H). 1H of | | | | | | |
| | | 2HCl was not observed. | | | | | | |
| 569 | 2HCl | ¹H NMR (400 MHz, DMSO) δ 9.69 | DMSO | >98 | Method | | | |
| | | (d, J = 1.6 Hz, 1H), 9.23 (d, J = | | | R3 | | | |
| | | 8.0 Hz, 1H), 9.01 (d, J = 4.3 Hz, | | | | | | |
| | | 1H), 8.93 (d, J = 5.4 Hz, 1H), 8.59 | | | | | | |
| | | (s, 1H), 8.24 (dd, J = 12.0, 1.6 Hz, | | | | | | |
| | | 1H), 8.03-7.90 (m, 1H), 7.73 (d, | | | | | | |
| | | J = 7.1 Hz, 2H), 7.42-7.18 (m, | | | | | | |
| | | 1H), 3.23 (d, J = 4.5 Hz, 3H). 1H | | | | | | |
| | | of 2HCl was not observed. | | | | | | |
| 570 | 2HCl | ¹H NMR (400 MHz, DMSO) δ 9.69 | DMSO | >98 | Method | | | |
| | | (d, J = 1.7 Hz, 1H), 9.23 (d, J = | | | R3 | | | |
| | | 7.5 Hz, 1H), 9.00 (d, J = 4.3 Hz, | | | | | | |
| | | 1H), 8.93 (d, J = 5.3 Hz, 1H), 8.60 | | | | | | |
| | | (s, 1H), 8.40 (s, 1H), 8.25 (dd, J = | | | | | | |
| | | 15.0, 4.8 Hz, 2H), 8.02-7.96 (m, | | | | | | |
| | | 1H), 7.92 (d, J = 7.8 Hz, 1H), 7.77 | | | | | | |
| | | (t, J = 7.8 Hz, 1H), 3.24 (d, J = | | | | | | |
| | | 4.5 Hz, 3H). 1H of 2HCl was not | | | | | | |
| | | observed. | | | | | | |
| 571 | 2HCl | ¹H NMR (400 MHz, DMSO) δ 9.69 | DMSO | >98 | Method | | | |
| | | (s, 1H), 9.22 (s, 1H), 9.06 (s, 1H), | | | R3 | | | |
| | | 8.92 (d, J = 5.2 Hz, 1H), 8.63 (s, | | | | | | |
| | | 1H), 8.23 (d, J = 12.1 Hz, 1H), | | | | | | |
| | | 8.10 (d, J = 8.4 Hz, 2H), 8.04 (d, J = | | | | | | |
| | | 8.4 Hz, 2H), 7.97 (s, 1H), 3.23 | | | | | | |
| | | (d, J = 4.4 Hz, 3H). 1H of 2HCl | | | | | | |
| | | was not observed. | | | | | | |
| 572 | 2HCl | ¹H NMR (400 MHz, DMSO) δ 9.69 | DMSO | >98 | Method | | | |
| | | (d, J = 1.5 Hz, 1H), 9.15 (d, J = | | | R3 | | | |
| | | 6.3 Hz, 1H), 8.94-8.78 (m, 2H), | | | | | | |
| | | 8.38 (s, 1H), 8.01-7.84 (m, 2H), | | | | | | |
| | | 7.62 (ddd, J = 9.2, 6.2, 3.2 Hz, | | | | | | |
| | | 1H), 7.54-7.43 (m, 1H), 7.42- | | | | | | |
| | | 7.30 (m, 1H), 3.21 (d, J = 4.5 Hz, | | | | | | |
| | | 3H). 1H of 2HCl was not observed. | | | | | | |
| 573 | 2HCl | ¹H NMR (400 MHz, DMSO) δ 9.68 | DMSO | >98 | Method | | | |
| | | (s, 1H), 9.25 (d, J = 8.2 Hz, 1H), | | | R3 | | | |
| | | 8.95 (dd, J = 14.3, 4.4 Hz, 2H), | | | | | | |
| | | 8.45 (s, 1H), 8.08 (dd, J = 12.2, | | | | | | |
| | | 1.6 Hz, 1H), 8.04-7.93 (m, 1H), | | | | | | |
| | | 7.87 (d, J = 8.9 Hz, 2H), 7.11 (d, J = | | | | | | |
| | | 8.9 Hz, 2H), 3.84 (s, 3H), 3.22 | | | | | | |
| | | (d, J = 4.5 Hz, 3H). 1H of 2HCl | | | | | | |
| | | was not observed. | | | | | | |
| 574 | 2HCl | ¹H NMR (400 MHz, DMSO) δ 9.69 | DMSO | >98 | Method | | | |
| | | (d, J = 1.6 Hz, 1H), 9.26 (d, J = | | | R3 | | | |
| | | 8.1 Hz, 1H), 9.02 (d, J = 4.8 Hz, | | | | | | |
| | | 1H), 8.94 (d, J = 4.1 Hz, 1H), 8.51 | | | | | | |
| | | (s, 1H), 8.14 (dd, J = 12.1, 1.5 Hz, | | | | | | |
| | | 1H), 8.02 (dd, J = 7.8, 5.4 Hz, | | | | | | |
| | | 1H), 7.53-7.38 (m, 3H), 7.03 (dt, | | | | | | |
| | | J = 6.6, 2.5 Hz, 1H), 3.88 (s, 3H), | | | | | | |
| | | 3.23 (d, J = 4.5 Hz, 3H). 1H of | | | | | | |
| | | 2HCl was not observed. | | | | | | |
| 575 | HCl | ¹H NMR (400 MHz, DMSO) δ 9.69 | DMSO | >98 | Method | | | |
| | | (d, J = 1.6 Hz, 1H), 9.12 (d, J = | | | R6 | | | |
| | | 7.3 Hz, 1H), 8.91-8.80 (m, 2H), | | | Base: Et3N | | | |
| | | 8.27 (s, 1H), 7.87 (dd, J = 12.1, | | | Solvent: EtOH | | | |
| | | 6.6 Hz, 2H), 7.58 (dd, J = 15.8, | | | | | | |
| | | 7.5 Hz, 1H), 7.33 (t, J = 8.1 Hz, | | | | | | |
| | | 2H), 3.18 (d, J = 4.5 Hz, 3H). 1H | | | | | | |
| | | of HCl was not observed. | | | | | | |

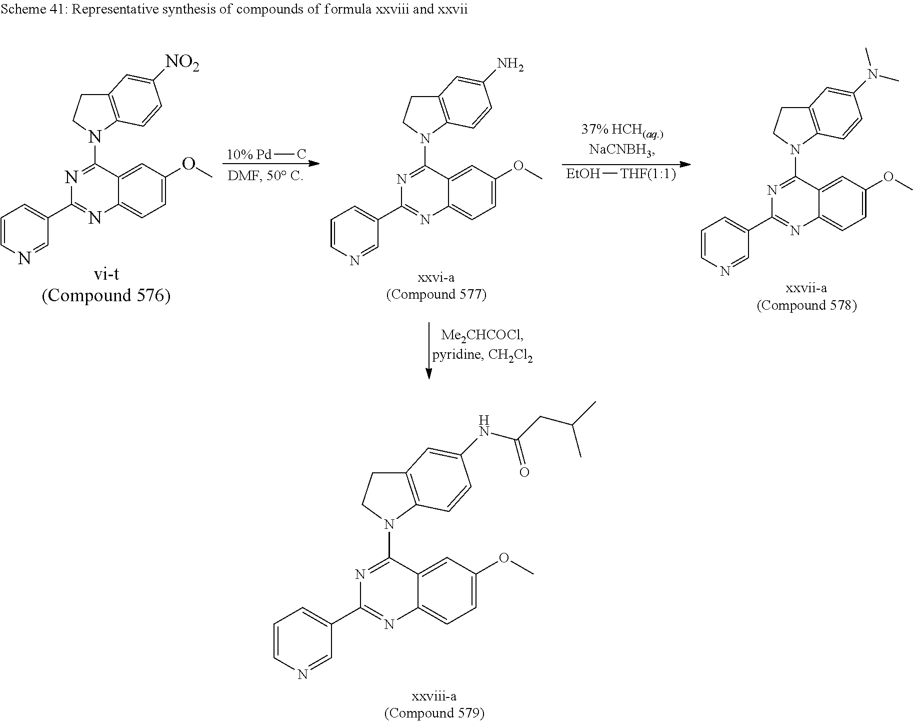

### 1-(6-methoxy-2-(pyridin-3-yl)quinazolin-4-yl)indolin-5-amine trihydrochloride (xxvi-a, compound 577)

To a solution of 6-methoxy-4-(5-nitroindolin-1-yl)-2-(pyridin-3-yl)quinazoline (2.0 g, 5.01 mmol) in DMF (30 ml) was added 10% Pd—C (0.3 g). The reaction was stirred for 3 h under a H₂ atmosphere. The reaction mixture was diluted with ethyl acetate (50 mL) and filtered to remove the catalyst. The organic layer was washed with H₂O (30 mL×2) and brine and then dried over Na₂SO₄. The organics were concentrated under reduced pressure to give the desired compound as a light yellow solid. The product was treated with a small excess of 5N HCl (1.0 mL) to form the HCl salt. The salt was filtered and washed with ethanol to give 1-(6-methoxy-2-(pyridin-3-yl)quinazolin-4-yl)indolin-5-amine trihydrochloride (2.0 g, 83.4%) as a pale brown powder. ¹H NMR (400 MHz, DMSO) δ 10.75-9.99 (m, 2H), 9.55 (d, J=1.8 Hz, 1H), 9.18 (d, J=8.3 Hz, 1H), 8.96 (dd, J=5.4, 1.4 Hz, 1H), 8.04 (t, J=6.7 Hz, 2H), 7.77 (d, J=8.5 Hz, 1H), 7.69 (dd, J=9.2, 2.7 Hz, 1H), 7.50 (d, J=2.7 Hz, 1H), 7.39 (s, 1H), 7.31 (dd, J=8.5, 2.1 Hz, 1H), 4.70 (t, J=8.0 Hz, 2H), 3.92 (s, 3H), 3.29 (t, J=7.9 Hz, 2H).

### 1-(6-methoxy-2-(pyridin-3-yl)quinazolin-4-yl)-N,N-dimethylindolin-5-amine trihydrochloride (xxvii-a, compound 578)

To a solution of 1-(6-methoxy-2-(pyridin-3-yl)quinazolin-4-yl)indolin-5-amine (300 mg, 0.812 mmol) in methanol-THF (10 ml, 1:1) was added 37% formaldehyde (0.605 ml, 8.12 mmol) and acetic acid (0.1 ml, 0.812 mmol) followed by sodium cyanoborohydride (255 mg, 4.06 mmol) at 0° C. The mixture was stirred for 2 d and then diluted with H₂O. The aqueous solution was extracted with CH₂Cl₂ (30 mL×2) and the combined organic layers were washed with brine, dried over Na₂SO₄ and filtered. The crude product was purified using SiO₂-chromatograghy (hexane:ethyl acetate 5:1) to give the free base in 0.20 g as an yellow amorphous. The desired product was treated with a small excess of 5N HCl_{(aq.)} (0.5 mL) to form the HCl salt. The salt was filtered and washed with ethanol to give 1-(6-methoxy-2-(pyridin-3-yl)quinazolin-4-yl)-N,N-dimethylindolin-5-amine trihydrochloride (0.19 g, 46.2% yield) as a pale brown powder. ¹H NMR (400 MHz, DMSO) δ 9.56 (d, J=1.9 Hz, 1H), 9.21 (d, J=8.4 Hz, 1H), 8.99 (dd, J=5.4, 1.3 Hz, 1H), 8.08 (t, J=8.0 Hz, 2H), 7.88-7.66 (m, 3H), 7.66-7.56 (m, 1H), 7.52 (d, J=2.4 Hz, 1H), 4.73 (t, J=7.9 Hz, 2H), 3.93 (s, 3H), 3.30 (t, J=7.8 Hz, 2H), 3.15 (s, 6H).

### N-(1-(6-methoxy-2-(pyridin-3-yl)quinazolin-4-yl)indolin-5-yl)-3-methylbutanamide (xxviii-a, compound 579)

To a solution of 1-(6-methoxy-2-(pyridin-3-yl)quinazolin-4-yl)indolin-5-amine (0.30 g, 0.812 mmol) and pyridine (0.131 ml, 1.624 mmol) in CH₂Cl₂ (5 ml) was added 3-methyl-butanoyl chloride (0.109 ml, 0.893 mmol) dropwise at 0° C. The mixture was stirred for 2 h and diluted with H₂O. The organics were evaporated off to give an aqueous suspension which was filtered and washed with ether to give a yellow powder of N-(1-(6-methoxy-2-(pyridin-3-yl)quinazolin-4-yl)indolin-5-yl)-3-methylbutanamide (0.27 g, 73.3% yield) a pale brown powder. ¹H NMR (400 MHz, CDCl₃) δ 9.71 (d, J=1.6 Hz, 1H), 8.82-8.74 (m, 1H), 8.67 (dd, J=4.8, 1.5 Hz, 1H), 7.96 (d, J=9.2 Hz, 1H), 7.73 (s, 1H), 7.52-7.45 (m, 1H), 7.40 (dd, J=7.9, 4.7 Hz, 1H), 7.24 (d, J=2.7 Hz, 1H), 7.16 (s, 1H), 7.09 (d, J=8.6 Hz, 1H), 7.03 (dd, J=8.5, 2.0 Hz, 1H), 4.51 (t, J=8.0 Hz, 2H), 3.81 (d, J=5.7 Hz, 3H), 3.25 (t, J=8.0 Hz, 2H), 2.24 (t, J=5.8 Hz, 3H), 1.61 (s, 2H), 1.09-0.97 (m, 6H).

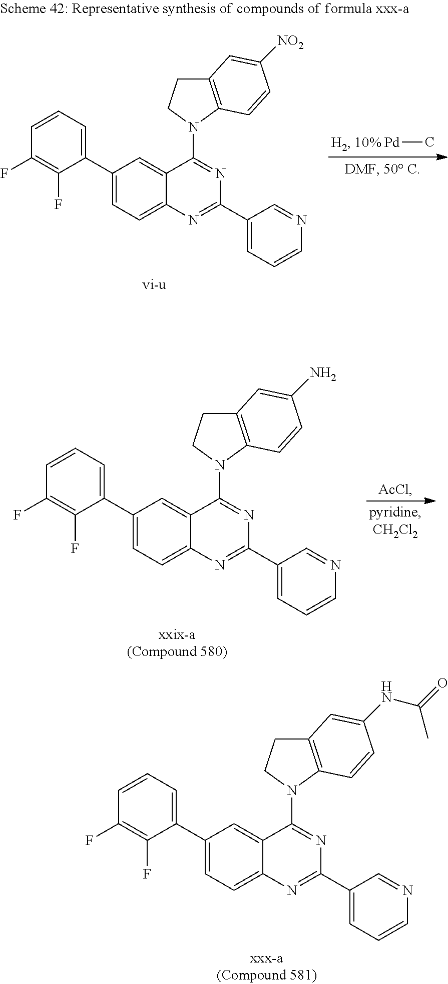

### 1-(6-(2,3-difluorophenyl)-2-(pyridin-3-yl)quinazolin-4-yl)indolin-5-amine (xxix-a, compound 580)

To a solution of 6-(2,3-difluorophenyl)-4-(5-nitroindolin-1-yl)-2-(pyridin-3-yl)quinazoline (0.2 g, 0.415 mmol) in DMF (5 ml) was added 10% Pd—C (0.1 g). The reaction was stirred for 5 h at 50° C. under H₂ atmosphere. The reaction mixture was filtered to remove the palladium catalyst and diluted with ethyl acetate. The organic layer was washed with H₂O (30 mL×2) and brine and then dried over Na₂SO₄. The organics were concentrated under reduced pressure to give the desired compound, 1-(6-(2,3-difluorophenyl)-2-(pyridin-3-yl)quinazolin-4-yl)indolin-5-amine, (0.15 g, 0.33 mmol, 80.0% yield) as a brown powder. ¹H NMR (400 MHz, CDCl₃) δ 9.73 (d, J=1.5 Hz, 1H), 8.80 (dt, J=8.0, 1.9 Hz, 1H), 8.69 (dd, J=4.8, 1.7 Hz, 1H), 8.29 (s, 1H), 8.02 (d, J=4.2 Hz, 2H), 7.93 (dt, J=8.7, 1.6 Hz, 1H), 7.57 (d, J=8.5 Hz, 1H), 7.41 (dd, J=7.9, 4.8 Hz, 1H), 7.29-7.09 (m, 2H), 6.73-6.55 (m, 2H), 4.56 (t, J=7.8 Hz, 2H), 3.64 (brs, 2H), 3.23-3.13 (m, 2H).

### N-(1-(6-(2,3-difluorophenyl)-2-(pyridin-3-yl)quinazolin-4-yl)indolin-5-yl)acetamide dihydrochloride (xxx-a, compound 581)

To a solution of 1-(6-(2,3-difluorophenyl)-2-(pyridin-3-yl)quinazolin-4-yl)indolin-5-amine (0.14 g, 0.310 mmol) and pyridine (0.075 ml, 0.930 mmol) in CH₂Cl₂ (10 ml) was added acetyl chloride (0.066 ml, 0.930 mmol) dropwise at 0° C. The reaction was stirred for 15 h and then washed with water and brine, dried over Na₂SO₄ and filtered. The crude product was treated with a small excess of 5N HCl_{(aq.)} (1.0 mL) to form the HCl salt. The salt was filtered and recrystallized from ethanol to give N-(1-(6-(2,3-difluorophenyl)-2-(pyridin-3-yl)quinazolin-4-yl)indolin-5-yl)acetamide dihydrochloride (80 mg, 45.6% yield). ¹H NMR (400 MHz, DMSO) δ 10.16 (s, 1H), 9.59 (d, J=1.8 Hz, 1H), 9.17 (d, J=8.0 Hz, 1H), 8.99 (dd, J=5.4, 1.4 Hz, 1H), 8.48 (s, 1H), 8.18 (s, 2H), 8.09-7.97 (m, 2H), 7.72 (s, 1H), 7.62-7.48 (m, 3H), 7.38 (dd, J=13.2, 8.0 Hz, 1H), 4.76 (t, J=7.6 Hz, 2H), 3.24 (t, J=7.6 Hz, 2H), 2.08 (s, 3H).

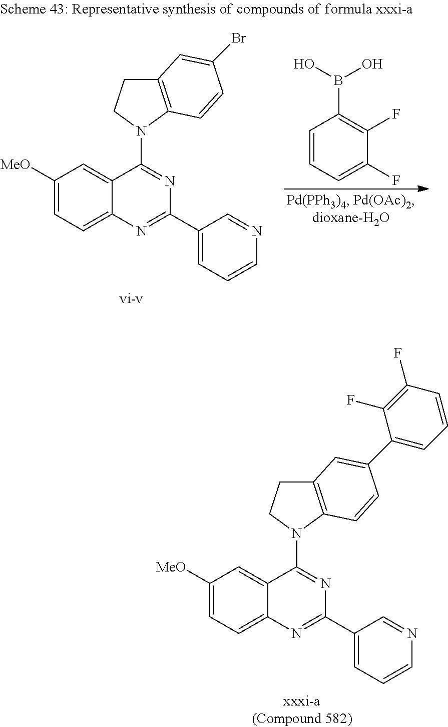 [SMILES: COc1ccc2nc(-c3cccnc3)nc(N3CCc4cc(Br)ccc43)c2c1 ; OB(O)c1cccc(F)c1F ; COc1ccc2nc(-c3cccnc3)nc(N3CCc4cc(-c5cccc(F)c5F)ccc43)c2c1]

### 4-(5-(2,3-difluorophenyl)indolin-1-yl)-6-methoxy-2-(pyridin-3-yl)quinazoline (xxxi-a, compound 582)

To a mixture of 4-(5-bromoindolin-1-yl)-6-methoxy-2-(pyridin-3-yl)quinazoline (0.10 g, 0.231 mmol) in dioxane-H₂O (12 ml 5:1) were added 2,3-difluorobenzeneboronic acid (0.055 g, 0.346 mmol), K₃PO₄ (0.147 g, 0.692 mmol) and Pd(Ph₃P)₄ (0.027 g, 0.023 mmol). The reaction was stirred under N₂ at 90˜100° C. for 5 h. The reaction mixture was diluted with ethyl acetate and washed with water and brine, dried over Na₂SO₄ and filtered. The filtrate was concentrated down to give a yellow powder which was washed with ether to afford 4-(5-(2,3-difluorophenyl)indolin-1-yl)-6-methoxy-2-(pyridin-3-yl)quinazoline (60 mg, 55.7% yield). ¹H NMR (400 MHz, DMSO) δ 9.61-9.54 (m, 1H), 8.74-8.65 (m, 2H), 7.96 (d, J=9.2 Hz, 1H), 7.68-7.52 (m, 4H), 7.51-7.36 (m, 4H), 7.35-7.24 (m, 1H), 4.64 (t, J=8.1 Hz, 2H), 3.89 (s, 3H), 3.39-3.23 (m, 2H).

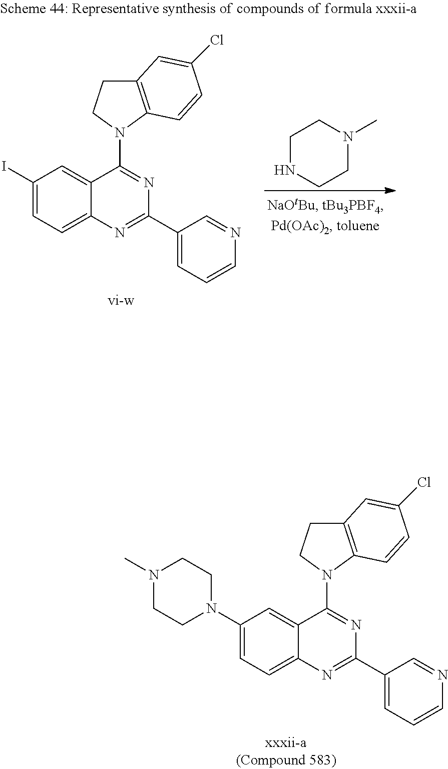

### 4-(5-chloroindolin-1-yl)-6-(4-methylpiperazin-1-yl)-2-(pyridin-3-yl)quinazoline dihydrochloride (xxxii-a, compound 583)

A mixture of 4-(5-chloroindolin-1-yl)-6-iodo-2-(pyridin-3-yl)quinazoline (0.4 g, 0.825 mmol), 1-methyl piperazine (0.099 g, 0.990 mmol), tri(tert-butylphosphonium)tetrafluoroborate (0.024 g, 0.083 mmol), sodium-t-butoxide (0.101 ml, 1.155 mmol) and palladium (II) acetate (0.019 g, 0.083 mmol) in toluene (15 ml) was stirred for 5 hr at 100° C. The reaction mixture was filtered through celite to remove the palladium black and concentrated in vacuo. The resulting residue was purified using NH—SiO₂-chromatography (hexane:ethyl acetate=5:1-1:1) to give the parent which was treated with a small excess of 5N HCl_{(aq)} (1.0 ml) to give 4-(5-chloroindolin-1-yl)-6-(4-methylpiperazin-1-yl)-2-(pyridin-3-yl)quinazoline dihydrochloride (0.18 g, 41.2% yield) as an orange solid. ¹H NMR (400 MHz, CDCl₃) δ 9.70 (d, J=2.0 Hz, 1H), 8.84-8.72 (m, 1H), 8.67 (dd, J=4.8, 1.7 Hz, 1H), 7.94 (d, J=9.3 Hz, 1H), 7.62 (dd, J=9.3, 2.6 Hz, 1H), 7.39 (dd, J=8.0, 4.8 Hz, 1H), 7.26 (s, 1H), 7.15-7.00 (m, 2H), 6.93 (dd, J=22.2, 8.6 Hz, 1H), 4.50 (t, J=8.0 Hz, 2H), 3.34-3.20 (m, 6H), 2.58 (dd, J=17.9, 13.0 Hz, 4H), 2.37 (s, 3H).

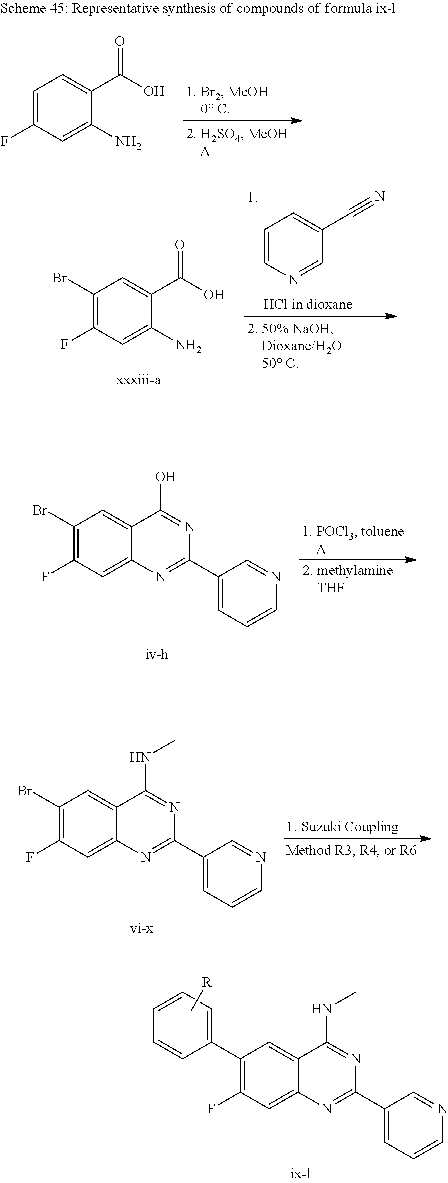

### Methyl 2-amino-5-bromo-4-fluorobenzoate (xxxiii-a)

To a solution of 2-amino-4-fluorobenzoic acid (7.73 g, 49.8 mmol) in methanol (120 ml) was added bromine (3.1 ml, 60.2 mmol) at 0° C. The reaction was stirred at 0° C. for 1 h and then warmed to room temperature and stirred for an additional 2 h. The reaction mixture was concentrated in vacuo to give the crude product. The resulting product was then dissolved in methanol (240 ml) and conc. H₂SO₄ (34 ml, 638 mmol) was added dropwise to the reaction mixture at 0° C. and then refluxed overnight. The methanol was evaporated off until ca. 1/3 volume. Then, 5N NaOH aq. (260 mL) was added to the solution at 0° C. and extracted with ethyl acetate. The organics were collected and dried over Na₂SO₄, filtered and concentrated. The crude product was purified using NH-silica-gel to give 2.82 g of methyl 2-amino-5-bromo-4-fluorobenzoate. ¹H NMR (400 MHz, DMSO) δ 7.90 (d, J=8.1 Hz, 1H), 7.01 (s, 2H), 6.72 (d, J=11.5 Hz, 1H), 3.79 (s, 3H).

### 6-Bromo-7-fluoro-2-(pyridin-3-yl)quinazolin-4-ol (iv-h)

To a suspension of methyl 2-amino-5-bromo-4-fluorobenzoate (2.82 g, 11.37 mmol) in saturated HCl in dioxane (100 mL) was added 3-cyanopyridine (2.60 g, 25.01 mmol) at 0° C. The reaction was stirred at room temperature overnight. The mixture was diluted with ether (100 ml) and stirred at room temperature for 1 h. The resultant precipitate was filtered and washed with Et₂O to give the crude product. This material was used directly in the next reaction by suspending in dioxane (40 ml)/H₂O (40 ml). A 50% NaOH_{(aq)} solution (10 ml) was added and stirred at 50° C. for 3 h. 5 N HCl_{(aq)} (30 ml) was added at 0° C. followed by H₂O (ca. 150 ml). The mixture was stirred at room temperature for 20 min and the desired product was collected by filtration and washed with H₂O, dried in an oven at 60° C. overnight to give 3.367 g of 6-bromo-7-fluoro-2-(pyridin-3-yl)quinazolin-4-ol. ¹H NMR (400 MHz, DMSO) δ 12.98 (s, 1H), 9.28 (s, 1H), 8.78 (d, J=4.2 Hz, 1H), 8.48 (d, J=8.0 Hz, 1H), 8.39 (d, J=7.6 Hz, 1H), 7.76 (d, J=9.7 Hz, 1H), 7.61 (dd, J=7.9, 4.8 Hz, 1H).

### 6-Bromo-7-fluoro-N-methyl-2-(pyridin-3-yl)quinazolin-4-amine(vi-x)

6-Bromo-7-fluoro-2-(pyridin-3-yl)quinazolin-4-ol (3.367 g, 10.52 mmol) was suspended in toluene (40 mL), and POCl₃ (6 mL, 64.4 mmol) was added and refluxed for 2 h. The reaction mixture was concentrated down to give the crude product which was used directly in the next reaction. The solid was mixed with THF (40 ml), and 40% aqueous solution of methylamine (23 mL, 267 mmol) was added at 0° C. slowly. The mixture was stirred at room temperature for 12 h and concentrated. The precipitate was stirred with H₂O (100 ml)/methanol (50 ml) for 2 h. The resulting solid was collected by filtration and washed with H₂O, dried in vacuo to give 3.49 g of 6-bromo-7-fluoro-N-methyl-2-(pyridin-3-yl)quinazolin-4-amine. ¹H NMR (400 MHz, DMSO) δ 9.61 (d, J=1.4 Hz, 1H), 8.88-8.51 (m, 4H), 7.71 (d, J=10.1 Hz, 1H), 7.55 (dd, J=8.0, 4.8 Hz, 1H), 3.15 (d, J=4.5 Hz, 3H).

### 7-fluoro-6-(3-fluorophenyl)-N-methyl-2-(pyridin-3-yl)quinazolin-4-amine (ix-l)

7-fluoro-6-(3-fluorophenyl)-N-methyl-2-(pyridin-3-yl)quinazolin-4-amine was prepared from 6-bromo-7-fluoro-N-methyl-2-(pyridin-3-yl)quinazolin-4-amine and 3-fluorophenylboronic acid in a manner analogous to that described for 6-(6-methoxypyridin-3-yl)-N-methyl-2-(pyridine-3-yl)quinazoline-4-amine using Method R6 substituting for the appropriate base and catalyst from method R2 and substituting 3-fluorophenylboronic acid for 6-methoxypyridin-3-ylboronic acid

The compounds in the following table were prepared in a manner analogous to that described in Scheme 45.

TABLE 11

| | | | | | | | | Method | | Reten- | |
|---|---|---|---|---|---|---|---|---|---|---|---|
| | Starting | Starting | | Salt | | ¹H NMR | Purity | of | | tion | LCMS |
| Number | Material 1 | Material 2 | Product | type | ¹H NMR | Solvent | percent | Coupling | LCMS | Time | Method |
| 584 | 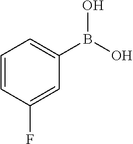 | 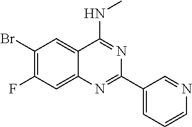 [SMILES: CNc1nc(-c2cccnc2)nc2cc(F)c(Br)cc12] | 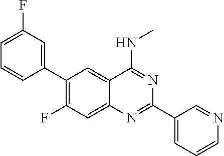 [SMILES: CNc1nc(-c2cccnc2)nc2cc(F)c(-c3cccc(F)c3)cc12] | 2HCl | . ¹H NMR (400 MHz, DMSO) δ 9.66 (s, 1H), 9.31 (s, 1H), 9.10 (d, J = 6.5 Hz, 1H), 8.92 (d, J = 4.6 Hz, 1H), 8.65 (d, J = 7.9 Hz, 1H), 7.98-7.84 (m, 1H), 7.79 (d, J = 11.3 Hz, 1H), 7.69-7.49 (m, 3H), 7.36 (t, J = | DMSO | >98 | Method R6 | | | |
| | | | | | 9.2 Hz, 1H), 3.23 (d, | | | | | | |
| | | | | | J = 4.3 Hz, 3H). 1H of | | | | | | |
| | | | | | 3HCl was not observed. | | | | | | |
| 585 | 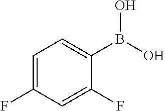 [SMILES: OB(O)c1ccc(F)cc1F] | 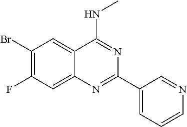 [SMILES: CNc1nc(-c2cccnc2)nc2cc(F)c(Br)cc12] | 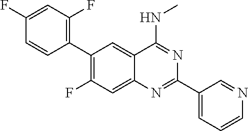 [SMILES: CNc1nc(-c2cccnc2)nc2cc(F)c(-c3ccc(F)cc3F)cc12] | 2HCl | ¹H NMR (400 MHz, DMSO) δ 9.67 (d, J = 1.6 Hz, 1H), 9.47- 9.24 (m, 1H), 9.18 (d, J = 7.6 Hz, 1H), 8.96 (dd, J = 5.3, 1.5 Hz, 1H), 8.59 (d, J = 7.5 Hz, 1H), 8.02- | DMSO | >98 | Method R6 | | | |
| | | | | | 7.90 (m, 1H), 7.85 | | | | | | |
| | | | | | (d, J = 10.9 Hz, 1H), | | | | | | |
| | | | | | 7.68 (dd, J = 15.2, | | | | | | |
| | | | | | 8.6 Hz, 1H), 7.60- | | | | | | |
| | | | | | 7.43 (m, 1H), 7.35 | | | | | | |
| | | | | | (td, J = 8.5, 2.8 Hz, | | | | | | |
| | | | | | 1H), 3.22 (d, J = 4.5 | | | | | | |
| | | | | | Hz, 3H). 2HCl was | | | | | | |
| | | | | | not observed. | | | | | | |
| 586 | 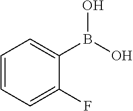 | 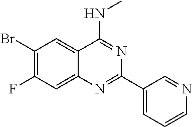 [SMILES: CNc1nc(-c2cccnc2)nc2cc(F)c(Br)cc12] | 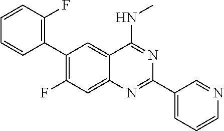 [SMILES: CNc1nc(-c2cccnc2)nc2cc(F)c(-c3ccccc3F)cc12] | HCl | ¹H NMR (400 MHz, DMSO) δ 9.65 (d, J = 1.5 Hz, 1H), 8.96 (d, J = 7.8 Hz, 1H), 8.96 (br, 1H), 8.83 (d, J = 5.0 Hz, 1H), 8.50 (d, J = 7.7 Hz, 1H), 7.77 (s, 1H), 7.71 (d, J = 11.0 Hz, 1H), 7.59 | DMSO | >98 | Method R6 | | | |
| | | | | | (dt, J = 7.9, 6.7 Hz, | | | | | | |
| | | | | | 2H), 7.42 (t, J = 8.1 | | | | | | |
| | | | | | Hz, 2H), 3.19 (d, J = | | | | | | |
| | | | | | 4.5 Hz, 3H). 1H of | | | | | | |
| | | | | | HCl was not observed. | | | | | | |
| 587 | 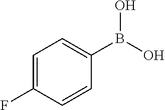 | 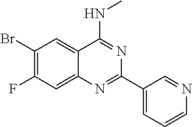 [SMILES: CNc1nc(-c2cccnc2)nc2cc(F)c(Br)cc12] | 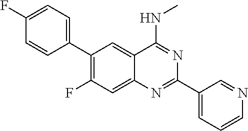 [SMILES: CNc1nc(-c2cccnc2)nc2cc(F)c(-c3ccc(F)cc3)cc12] | 2HCl | ¹H NMR (400 MHz, DMSO) δ 9.65 (d, J = 1.5 Hz, 1H), 9.31 (s, 1H), 9.09 (d, J = 7.5 Hz, 1H), 8.92 (dd, J = 5.2, 1.5 Hz, 1H), 8.61 (d, J = 8.1 Hz, 1H), 7.99-7.86 (m, | DMSO | >98 | Method R4 | | | |
| | | | | | 1H), 7.85-7.69 (m, | | | | | | |
| | | | | | 3H), 7.52-7.35 (m, | | | | | | |
| | | | | | 2H), 3.23 (d, J = | | | | | | |
| | | | | | 4.5 Hz, 3H). 1H of | | | | | | |
| | | | | | 2HCl was not observed. | | | | | | |
| 588 | 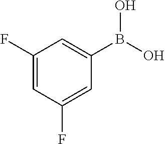 [SMILES: OB(O)c1cc(F)cc(F)c1] | 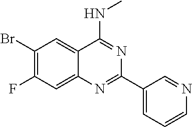 [SMILES: CNc1nc(-c2cccnc2)nc2cc(F)c(Br)cc12] | 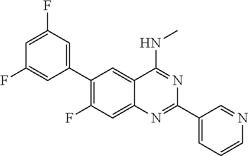 [SMILES: CNc1nc(-c2cccnc2)nc2cc(F)c(-c3cc(F)cc(F)c3)cc12] | 3HCl | ¹H NMR (400 MHz, DMSO) δ 9.67 (d, J = 1.6 Hz, 1H), 9.31 (s, 1H), 9.13 (d, J = 8.2 Hz, 1H), 8.95-8.90 (m, 1H), 8.69 (d, J = 8.2 Hz, 1H), 7.98- 7.88 (m, 1H), 7.81 (d, J = 12.0 Hz, 1H), 7.49 (d, J = 7.6 Hz, 2H), | DMSO | >98 | Method R4 | | | |
| | | | | | 7.46-7.37 (m, 1H), | | | | | | |
| | | | | | 3.23 (d, J = 4.5 Hz, | | | | | | |
| | | | | | 3H). 1H of 3HCl | | | | | | |
| | | | | | was not observed. | | | | | | |
| 589 | 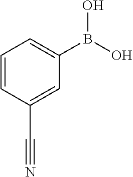 [SMILES: N#Cc1cccc(B(O)O)c1] | 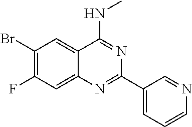 [SMILES: CNc1nc(-c2cccnc2)nc2cc(F)c(Br)cc12] | 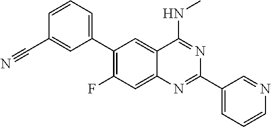 [SMILES: CNc1nc(-c2cccnc2)nc2cc(F)c(-c3cccc(C#N)c3)cc12] | | ¹H NMR (400 MHz, DMSO) δ 9.64 (d, J = 1.5 Hz, 1H), 8.82-8.75 (m, 1H), 8.71 (dd, J = 4.8, 1.6 Hz, 1H), 8.67 (d, J = 4.5 Hz, 1H), 8.55 (d, J = 8.4 Hz, 1H), 8.16 (s, 1H), 8.04 (d, J = 6.8 Hz, 1H), 7.96 (d, J = 7.8 Hz, 1H), 7.78 (t, J = | DMSO | >98 | Method R4 | | | |
| | | | | | 7.8 Hz, 1H), 7.68 (d, | | | | | | |
| | | | | | J = 12.2 Hz, 1H), | | | | | | |
| | | | | | 7.56 (dd, J = 7.9, 4.8 | | | | | | |
| | | | | | Hz, 1H), 3.18 (d, J = | | | | | | |
| | | | | | 4.5 Hz, 3H). | | | | | | |
| 590 | 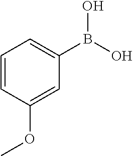 | 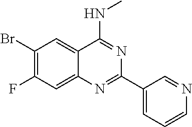 [SMILES: CNc1nc(-c2cccnc2)nc2cc(F)c(Br)cc12] | 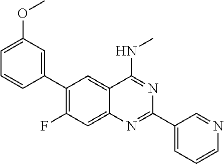 [SMILES: CNc1nc(-c2cccnc2)nc2cc(F)c(-c3cccc(OC)c3)cc12] | 2HCl | 1H NMR (400 MHz, DMSO) δ 9.65 (s, 1H), 9.35 (brs, 1H), 9.07 (s, 1H), 8.92 (d, J = 5.1 Hz, 1H), 8.63 (d, J = 8.0 Hz, 1H), 7.90 (s, 1H), 7.78 (d, J = 11.6 Hz, 1H), 7.49 (t, J = 8.0 Hz, 1H), 7.26 (d, J = 11.4 Hz, 2H), | DMSO | >98 | Method R4 | | | |
| | | | | | 7.08 (d, J = 8.0 Hz, | | | | | | |
| | | | | | 1H), 3.86 (s, 3H), | | | | | | |
| | | | | | 3.23 (d, J = 4.3 Hz, | | | | | | |
| | | | | | 3H). 1H of 2HCl | | | | | | |
| | | | | | was not observed. | | | | | | |
| 591 | 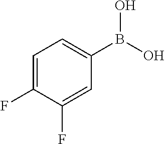 [SMILES: OB(O)c1ccc(F)c(F)c1] | 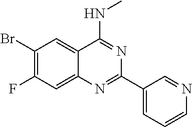 [SMILES: CNc1nc(-c2cccnc2)nc2cc(F)c(Br)cc12] | 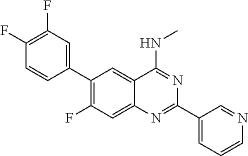 [SMILES: CNc1nc(-c2cccnc2)nc2cc(F)c(-c3ccc(F)c(F)c3)cc12] | 2HCl | ¹H NMR (400 MHz, DMSO) δ 9.65 (s, 1H), 8.99 (s, 2H), 8.85 (d, J = 5.1 Hz, 1H), 8.58 (d, J = 8.6 Hz, 1H), 7.80 (d, J = 7.8 Hz, 2H), 7.75-7.60 (m, 2H), 7.58 (s, 1H), 3.21 (d, J = 4.5 Hz, 3H). 1H of 2HCl | DMSO | >98 | Method R4 | | | |
| | | | | | was not observed. | | | | | | |
| 592 | 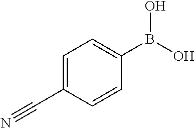 [SMILES: N#Cc1ccc(B(O)O)cc1] | 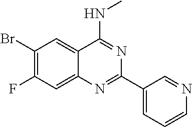 [SMILES: CNc1nc(-c2cccnc2)nc2cc(F)c(Br)cc12] | 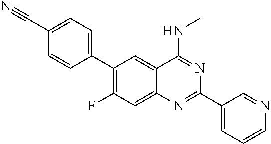 [SMILES: CNc1nc(-c2cccnc2)nc2cc(F)c(-c3ccc(C#N)cc3)cc12] | 2HCl | ¹H NMR (400 MHz, DMSO) δ 9.64 (d, J = 2.0 Hz, 1H), 8.80- 8.74 (m, 1H), 8.74- 8.67 (m, 2H), 8.57 (d, J = 8.4 Hz, 1H), 8.05 (d, J = 8.4 Hz, 2H), 7.90 (d, J = 7.0 Hz, 2H), 7.68 (d, | DMSO | >98 | Method R4 | | | |
| | | | | | J = 12.2 Hz, 1H), 7.56 | | | | | | |
| | | | | | (dd, J = 7.9, 4.8 Hz, | | | | | | |
| | | | | | 1H), 3.18 (d, J = 4.4 | | | | | | |
| | | | | | Hz, 3H). | | | | | | |
| 593 | 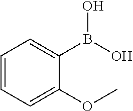 | 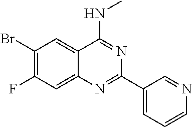 [SMILES: CNc1nc(-c2cccnc2)nc2cc(F)c(Br)cc12] | 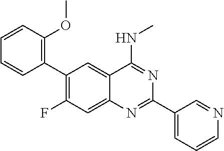 [SMILES: CNc1nc(-c2cccnc2)nc2cc(F)c(-c3ccccc3OC)cc12] | | ¹H NMR (400 MHz, DMSO) δ 9.64 (d, J = 1.6 Hz, 1H), 9.26- 9.05 (m, 1H), 9.00 (s, 1H), 8.89 (d, J = 3.6 Hz, 1H), 8.41 (d, J = 8.1 Hz, 1H), 7.85 (s, 1H), 7.69 (d, J = 10.2 Hz, 1H), 7.49 | DMSO | >98 | Method R4 | | | |
| | | | | | (dd, J = 11.3, 4.5 Hz, | | | | | | |
| | | | | | 1H), 7.37 (dd, J = 7.5, | | | | | | |
| | | | | | 1.7 Hz, 1H), 7.20 (d, | | | | | | |
| | | | | | J = 8.4 Hz, 1H), 7.12 | | | | | | |
| | | | | | (t, J = 7.0 Hz, 1H), | | | | | | |
| | | | | | 3.78 (s, 3H), 3.20 (d, | | | | | | |
| | | | | | J = 4.4 Hz, 3H). 1H | | | | | | |
| | | | | | of HCl was not | | | | | | |
| | | | | | observed. | | | | | | |
| 594 | 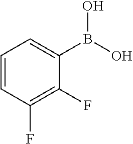 [SMILES: OB(O)c1cccc(F)c1F] | 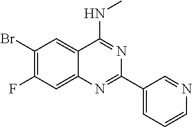 [SMILES: CNc1nc(-c2cccnc2)nc2cc(F)c(Br)cc12] | 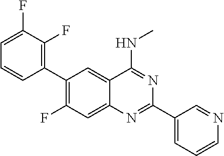 [SMILES: CNc1nc(-c2cccnc2)nc2cc(F)c(-c3cccc(F)c3F)cc12] | | ¹H NMR (400 MHz, DMSO) δ 9.66 (s, 1H), 9.12 (s, 1H), 9.06 (s, 1H), 8.89 (d, J = 5.2 Hz, 1H), 8.58 (d, J = 7.6 Hz, 1H), 7.87 (s, 1H), 7.79 (d, J = 11.0 Hz, 1H), 7.63 (dd, J = 17.9, 10.4 Hz, 1H), 7.44 (dd, J = | DMSO | >98 | Method R4 | | | |
| | | | | | 10.4, 7.0 Hz, 2H), | | | | | | |
| | | | | | 3.20 (d, J = 4.5 | | | | | | |
| | | | | | Hz, 3H). 1H of HCl | | | | | | |
| | | | | | was not observed. | | | | | | |

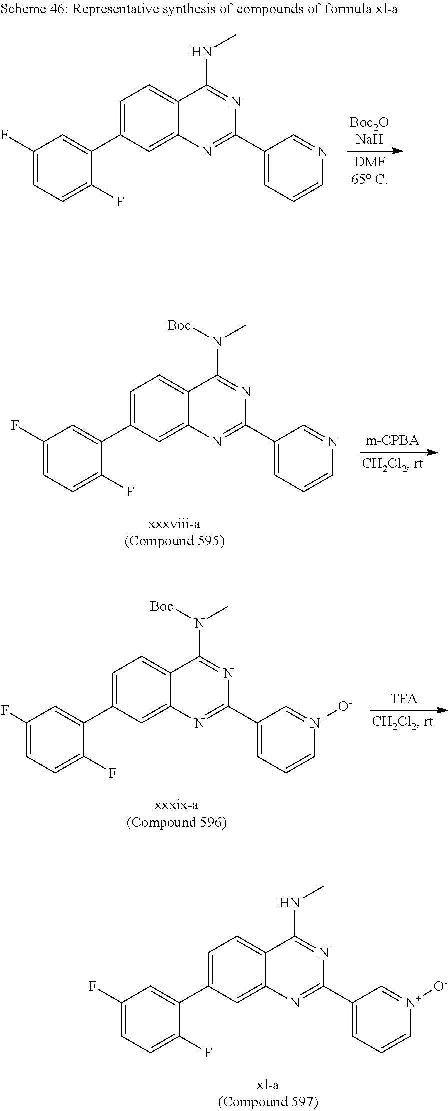 [SMILES: CNc1nc(-c2cccnc2)nc2cc(-c3cc(F)ccc3F)ccc12 ; CN(C(=O)OC(C)(C)C)c1nc(-c2cccnc2)nc2cc(-c3cc(F)ccc3F)ccc12 ; CN(C(=O)OC(C)(C)C)c1nc(-c2ccc[n+]([O-])c2)nc2cc(-c3cc(F)ccc3F)ccc12 ; CNc1nc(-c2ccc[n+]([O-])c2)nc2cc(-c3cc(F)ccc3F)ccc12]

### tert-Butyl 7-(2,5-difluorophenyl)-2-(pyridin-3-yl) quinazolin-4-yl(methyl)carbamate (xxxviii-a, compound 595)

A stirred solution of 7-(2,5-difluorophenyl)-N-methyl-2-(pyridin-3-yl)quinazolin-4-amine (synthesized in a similar manner described for 8-methoxy-6-(3-methoxyphenyl)-N-methyl-2-(pyridin-3-yl)quinazolin-4-amine substituting 7-bromo-N-methyl-2-(pyridin-3-yl)quinazolin-4-amine for 6-bromo-8-methoxy-N-methyl-2-(pyridin-3-yl)quinazolin-4-amine and 2,5-difluorophenylboronic acid for 3-methoxyphenylboronic acid) (1.00 g, 2.87 mmol) in DMF (60 ml) was added sodium hydride (55%, dispersion in paraffin liquid) (0.16 g, 3.73 mmol) at 0° C. The reaction was stirred at room temperature for 5 min, then di-tert-butyl dicarbonate (1.06 g, 4.88 mmol) was added to the suspension and stirred at room temperature for 3 h. After the reaction mixture was concentrated under reduced pressure, water was added to the residue and extracted with ethyl acetate. The organics were washed with brine, dried over MgSO₄, filtered and concentrated. The crude product was purified via ISCO(NH-silica gel, hexane/ethyl acetate=10/1-5/1) to afford 1.10 g (85% yield) of the desired product as a pale yellow amorphous. ¹H NMR (400 MHz, CDCl₃) δ 9.80 (dd, J=2.2, 0.8 Hz, 1H), 8.90-8.82 (m, 1H), 8.75 (dd, J=4.8, 1.7 Hz, 1H), 8.27-8.22 (m, 1H), 7.99 (dd, J=8.7, 0.4 Hz, 1H), 7.80-7.74 (m, 1H), 7.46 (ddd, J=8.0, 4.8, 0.8 Hz, 1H), 7.32 (ddd, J=8.8, 5.9, 3.1 Hz, 1H), 7.25-7.16 (m, 1H), 7.16-7.06 (m, 1H), 3.61 (s, 3H), 1.41 (s, 9H).

### 3-(4-(tert-Butoxycarbonylmethyl)amino)-7-(2,5-difluorophenyl)quinazolin-2-yl)pyridine 1-oxide (xxxix-a, compound 596)

To a solution of tert-butyl 7-(2,5-difluorophenyl)-2-(pyridin-3-yl) quinazolin-4-yl(methyl)carbamate (1.10 g, 2.45 mmol) in CH₂Cl₂ (50 mL) was added mCPBA (0.76 g, 4.4 mmol) at 0° C. The reaction mixture was stirred at room temperature for 3 h. After the reaction was completed, NH-silica-gel was added to the reaction mixture and concentrated. The silica-gel was placed directly on the ISCO column for purification (ISCO, NH-silica gel, ethyl acetate/methanol=1/0-20/1). The desired product was obtained as a white amorphous (1.08 g, 94% yield). ¹H NMR (400 MHz, CDCl₃) δ 9.47-9.39 (m, 1H), 8.55-8.43 (m, 1H), 8.33 (ddd, J=6.4, 1.8, 1.0 Hz, 1H), 8.27-8.18 (m, 1H), 8.00 (dd, J=8.7, 0.5 Hz, 1H), 7.84-7.76 (m, 1H), 7.49-7.40 (m, 1H), 7.31 (ddd, J=8.8, 5.9, 3.1 Hz, 1H), 7.26-7.17 (m, 1H), 7.17-7.08 (m, 1H), 3.59 (s, 3H), 1.42 (s, 9H).

### 3-(7-(2,5-difluorophenyl)-4-(N-methylamino)quinazolin-2-yl)pyridine 1-oxide (xl-a, compound 597)

To 3-(4-(tert-butoxycarbonyl(N-methyl)amino)-7-(2,5-difluorophenyl)quinazolin-2-yl)pyridine 1-oxide (500 mg, 1.07 mmol) in CH₂Cl₂ (3 ml) was added TFA (3 ml). The reaction was stirred at room temperature for 3 h. After the reaction was completed, volatiles were evaporated and aqueous NaHCO₃ solution was added to neutralize the reaction. The resulting precipitate was collected by filtration and that was dissolved in ethanol. To this was added NH-silica-gel and concentrated. The silica-gel was placed directly on the ISCO column for purification (ISCO, NH-silica gel, ethyl acetate/methanol=1/0-10/1). The appropriate fractions were concentrated to afford the desired product as a white solid. The product was washed with ethanol, filtered and dried by oven at 60° C. to afford the desired product as a white powder. ¹H NMR (400 MHz, DMSO) δ 9.06 (s, 1H), 8.74-8.60 (m, 1H), 8.44-8.27 (m, 3H), 7.98 (s, 1H), 7.80-7.71 (m, 1H), 7.64 (ddd, J=9.2, 6.0, 3.2 Hz, 1H), 7.60-7.51 (m, 1H), 7.51-7.40 (m, 1H), 7.40-7.27 (m, 1H), 3.17 (d, J=4.4 Hz, 3H).

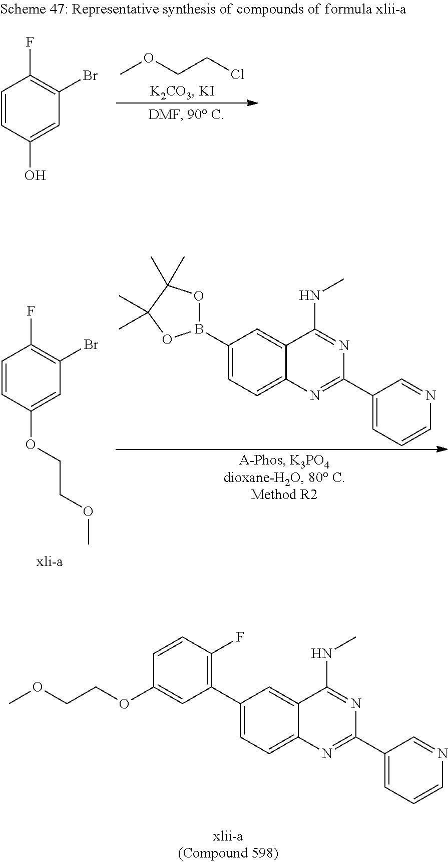

### 2-bromo-1-fluoro-4-(2-methoxyethoxy)benzene (xli-a)

A mixture of 3-bromo-4-fluorophenol (0.500 g, 2.62 mmol), 1-(2-chloroethoxy)methane (0.477 ml, 5.24 mmol), potassium carbonate (0.904 g, 6.54 mmol) and potassium iodide (0.956 g, 5.76 mmol) in DMF (10 mL) was stirred at 90° C. for 3 days. After being cooled to room temperature, the reaction mixture was diluted with water and ether. The organic layer was washed with brine, then dried over Na₂SO₄, filtrated and concentrated. The residue was purified via ISCO chromatography (silica gel, hexane:ethyl acetate=1:0 to 5:1) to give 0.51 g of the desired product as a colorless oil in 78% yield.

### Method R2: 6-(2-fluoro-5-(2-methoxyethoxy)phenyl)-N-methyl-2-(pyridin-3-yl)quinazolin-4-amine, dihydrochloride (xlii-a, compound 598)

A mixture of 2-bromo-1-fluoro-4-(2-methoxyethoxy)benzene (0.227 g, 0.911 mmol), N-methyl-2-(pyridin-3-yl)-6-(4,4,5,5-tetramethyl-1,3,2-dioxaborolan-2-yl)quinazolin-4-amine (0.300 g, 0.828 mmol), bis(di-tertbutyl(4-dimethylaminophenyephosphine)dichloropalladium(H) (0.047 g, 0.066 mmol), and potassium orthophosphate mono hydrate (0.572 g, 2.485 mmol) in 1,4-dioxane (10 ml) and water (1 ml) was stirred at 80° C. overnight under argon. After being cooled to room temperature, water (30 mL) and toluene (5 mL) were added to the reaction mixture. The resulted precipitate was filtered to give the desired compound as a free base. The HCl salt was formed by treatment with 4N HCl in dioxane (0.8 mL). The mixture was stirred at room temperature for 30 min and then concentrated in vacuo. The residue was crystallized from 2-propanol and water to give 110.8 mg of the desired product as a pale yellow powder in 28% yield. LCMS m/z=405 (M+1) (Method D) (retention time=1.53 min) ¹H NMR (300 MHz, DMSO) δ 10.19-9.45 (m, 2H), 9.14-8.83 (m, 2H), 8.65 (m, 1H), 8.31-8.00 (m, 2H), 7.83 (m, 1H), 7.48-7.19 (m, 2H), 7.07 (m, 1H), 4.31-4.02 (m, 2H), 3.88-3.57 (m, 2H), 3.31 (s, 3H), 3.26 (d, J=4.3 Hz, 3H).

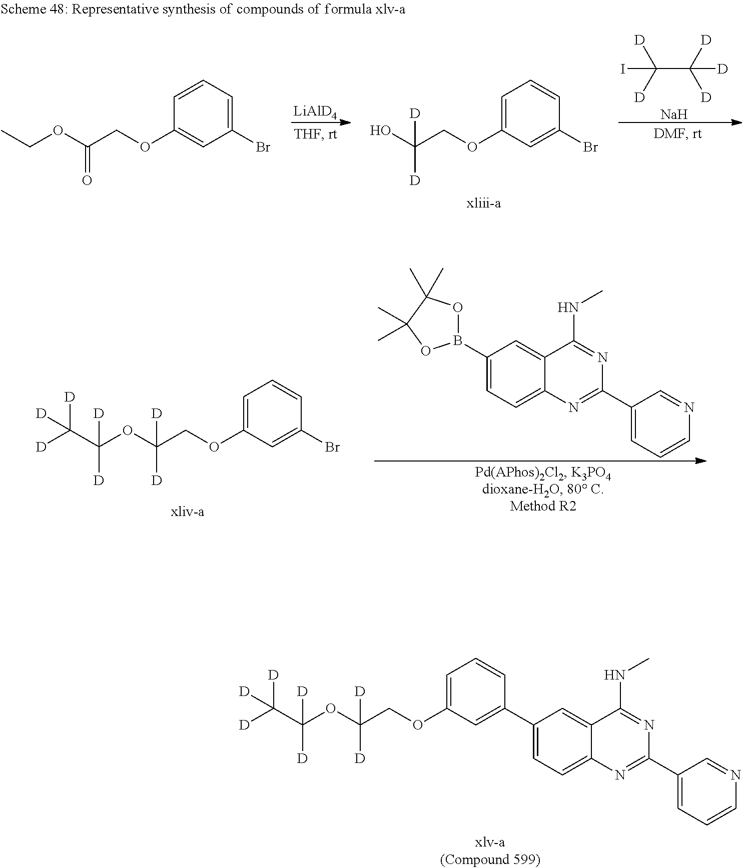

### 1-(3-bromophenoxy)ethan-2,2-d₂-2-ol (xliii-a)

To a solution of ethyl 2-(3-bromophenoxy)acetate (2.58 g, 9.94 mmol) in THF (30 mL) was added lithium aluminum deuteride (0.532 g, 12.66 mmol) at 0° C. After being stirred at room temperature for 30 min., a saturated solution of Na₂SO_{4 (aq.)} (1.7 mL) was added to the reaction at 0° C. The reaction was stirred for an additional 30 minutes and MgSO₄ was added and stirred for an additional 2 hour. The solid was removed by filtration through celite and the filtrate was concentrated in vacuo to give ˜1.5 g of a pale yellow oil (yield 69%) which was identified as the desired product by NMR analysis. ¹H NMR (300 MHz, CDCl₃) δ 7.24-7.05 (m, 3H), 6.86 (m, 1H), 4.07 (s, 2H), 1.86 (s, 1H).

### 1-bromo-3-(2-(ethoxy-d₅)-ethoxy-2,2-d2)benzene (xliv-a)

To a solution of 1-(3-bromophenoxy)ethan-2,2-d₂-2-ol (0.438 g, 1.998 mmol) in DMF (20 mL) were added iodoethane-d₅ (0.386 g, 2.398 mmol) and sodium hydride (0.092 g, 2.298 mmol) at 0° C. After being stirred at room temperature for 1 h, a saturated solution of NH₄Cl_{(aq.)} and ether were added to the mixture. The organic layer was washed with brine, dried over Na₂SO₄, filtrated and concentrated in vacuo. The residue was purified via ISCO chromatography (silica-gel, hexane:ethyl acetate=1:0 to 4:1). The fractions were collected to give 0.4 g of the desired product as a pale yellow oil in 79% yield. ¹H NMR (300 MHz, CDCl₃) δ 7.21-7.00 (m, 3H), 6.86 (m, 1H), 4.10 (s, 2H).

### 6-(3-(2-(Ethoxy-d₅-)ethoxy-2,2-d2-)phenyl)-N-methyl-2-(pyridin-3-yl)quinazolin-4-amine, dihydrochloride (xlv-a, compound 599)

A mixture of 1-bromo-3-(2-(ethoxy-d₅)-ethoxy-2,2-d₂)benzene (0.32 g, 1.269 mmol), N-methyl-2-(pyridin-3-yl)-6-(4,4,5,5-tetramethyl-1,3,2-dioxaborolan-2-yl)quinazolin-4-amine (0.383 g, 1.058 mmol), bis(di-tert-butyl(4-dimethylaminophenyl)phosphine)dichloropalladium(H) (0.060 g, 0.085 mmol), and potassium orthophosphate mono hydrate (0.731 g, 3.17 mmol) in 1,4-dioxane (10 ml) and water (1 ml) was stirred at 80° C. overnight under argon. After being cooled to room temperature, water (30 mL) and toluene (5 mL) were added to the reaction mixture. The resultant precipitate was filtered and purified via ISCO chromatography (silica-gel, CH₂Cl₂: ethyl acetate=1:0 to 1:9). The desired product was obtained as the free form and was converted to the HCl salt by suspending in dioxane (3 mL) and CH₂Cl₂ (3 mL) and adding a solution of HCl in dioxane (4M, 0.5 ml). The mixture was stirred at room temperature and then concentrated in vacuo. The product was recrystallized from 2-PrOH and water to give 0.267 g 6-(3-(2-(ethoxy-d₅-)ethoxy-2,2-d2-)phenyl)-N-methyl-2-(pyridin-3-yl)quinazolin-4-amine dihydrochloride as a pale yellow powder in 49% yield. LCMS m/z=408 (M+1) (Method D) (retention time=1.56 min) ¹H NMR (300 MHz, DMSO) δ 10.10 (br-s, 1H), 9.60 (s, 1H), 9.10-8.85 (m, 3H), 8.81 (s, 1H), 8.37 (d, J=8.8 Hz, 1H), 8.09 (d, J=8.9 Hz, 1H), 7.84 (m, 1H), 7.57-7.36 (m, 4H), 7.05 (m, 1H), 4.19 (s, 2H), 3.30 (d, J=4.3 Hz, 3H).

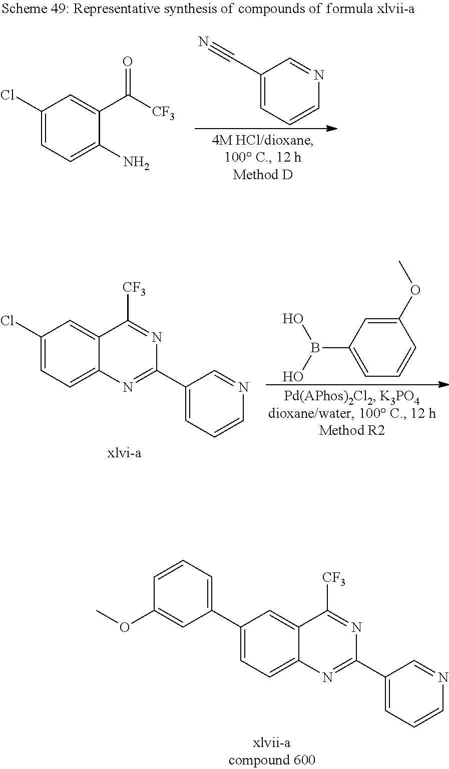 [SMILES: Nc1ccc(Cl)cc1C(=O)C(F)(F)F ; N#Cc1cccnc1 ; FC(F)(F)c1nc(-c2cccnc2)nc2ccc(Cl)cc12 ; COc1cccc(B(O)O)c1 ; COc1cccc(-c2ccc3nc(-c4cccnc4)nc(C(F)(F)F)c3c2)c1]

### Method D: 6-chloro-2-(pyridin-3-yl)-4-(trifluoromethyl)quinazoline (xlvi-a)

To a 75 mL sealed tube was added 1-(2-amino-5-chlorophenyl)-2,2,2-trifluoroethanone (2.0 g, 8.95 mmol) and 3-cyanopyridine (1.024 g, 9.84 mmol) in 4 M HCl/dioxane (30 mL) to give a tan solution. The reaction was heated at 100° C. overnight. LC-MS analysis of the crude mixture showed the reaction was completed. Upon cooling, the precipitate was collected as a yellow solid and washed with ethanol and ether. This crude product was isolated as the HCl salt, which was then free based by suspension in water followed by addition of 28% ammonium hydroxide until the pH of the mixture was ˜10. The suspension was stirred for 30 min, and then the precipitate was filtered to yield the desired compound as a white powder (0.82 g, 30%). LC-MS m/z=310.0 (M+1) (retention time=2.43) ¹H NMR (300 MHz, DMSO) δ 9.63 (d, J=1.3 Hz, 1H), 8.85-8.73 (m, 2H), 8.36-8.17 (m, 3H), 7.65 (dd, J=7.6, 5.2 Hz, 1H).

### Method R2: 6-(3-methoxyphenyl)-2-(pyridin-3-yl)-4-(trifluoromethyl)quinazoline, 2HCl (xlvii-a, compound 600)

To a 20 mL reaction vial were added 6-chloro-2-(pyridin-3-yl)-4-(trifluoromethyl)quinazoline (0.150 g, 0.484 mmol), 3-methoxyphenylboronic acid (0.098 g, 0.644 mmol), bis(di-tert-butyl(4-dimethylaminophenyl)phosphine)dichloropalladium(II) (10.29 mg, 0.015 mmol) and potassium phosphate tribasic monohydrate (0.335 g, 1.453 mmol) in dioxane (2 ml)/water (0.200 ml) to give a yellow suspension. The reaction was heated at 100° C. overnight. LC-MS analysis of the crude mixture showed the reaction was completed. To the reaction mixture was added water to yield a tan precipitate. The crude product was purified via ISCO (silica gel, 97:3 methylene chloride/methanol, 12 gm column). The fractions collected were concentrated and dried under vacuum to give a pale yellow powder. To form the salt, the material was suspended in methanol prior to the addition of 4 M HCl in dioxane. After stirring at ambient temperature for 2 h, the solvent was removed to give the desired product as an off-white solid (141.6 mg, 64%). LC-MS m/z=382.4 (M+1) (retention time=2.66) ¹H NMR (300 MHz, DMSO) δ 9.70 (d, J=2.1 Hz, 1H), 9.05 (d, J=8.1 Hz, 1H), 8.90 (dd, J=5.2, 1.3 Hz, 1H), 8.59 (dd, J=8.8, 1.6 Hz, 1H), 8.39 (d, J=8.9 Hz, 1H), 8.33 (s, 1H), 7.87 (dd, J=8.1, 5.1 Hz, 1H), 7.51 (t, J=7.9 Hz, 1H), 7.45-7.33 (m, 2H), 7.09 (dd, J=8.1, 2.4 Hz, 1H), 3.86 (s, J=12.1 Hz, 3H).

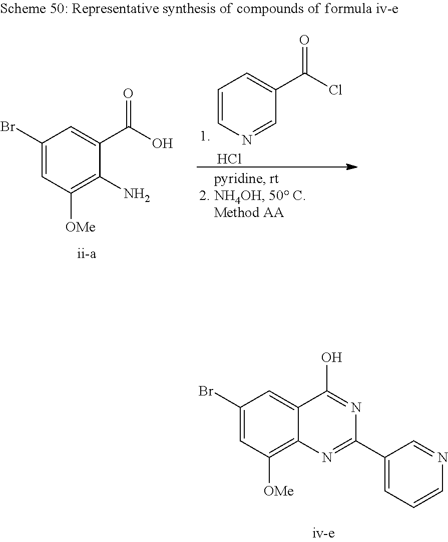 [SMILES: COc1cc(Br)cc(C(=O)O)c1N ; O=C(Cl)c1cccnc1 ; Cl ; COc1cc(Br)cc2c(O)nc(-c3cccnc3)nc12]

### Method AA: 6-bromo-8-methoxy-2-(pyridin-3-yl)quinazolin-4-ol (iv-e)

To a solution of 2-amino-5-bromo-3-methoxybenzoic acid hydrobromide (20 g, 0.061 moles, 1.0 equiv) in pyridine (250 mL) was added nicotinoyl chloride hydrochloride (32.7 g, 0.18, 3.0 equiv) over a period of 10 min, and the resultant mixture is allowed to stir at room temperature for 2 h. An ammonium hydroxide solution (80 mL) was added and the reaction was stirred for an additional 1 h at room temperature and then heated to 50° C. and stirred overnight to give a clear brown solution. After cooling to room temperature, the reaction mixture was poured into a vigorously stirred mixture of ether (500 mL)/ethanol (50 mL). The resultant precipitate was stirred for an additional 15-20 min and then collected by filtration. The crude product was washed with methanol and ether and then allowed to dry. The precipitate was triturated in water (250 mL) and stirred vigorously for 30-60 min. The precipitate was collected by filtration, washed with water, methanol and ether and then dried to give 6-bromo-8-methoxy-2-(pyridin-3-yl)quinazolin-4-ol as a white solid (14.8 g, 73%). LC-MS m/z=332.0 (M+1) (retention time=1.54).

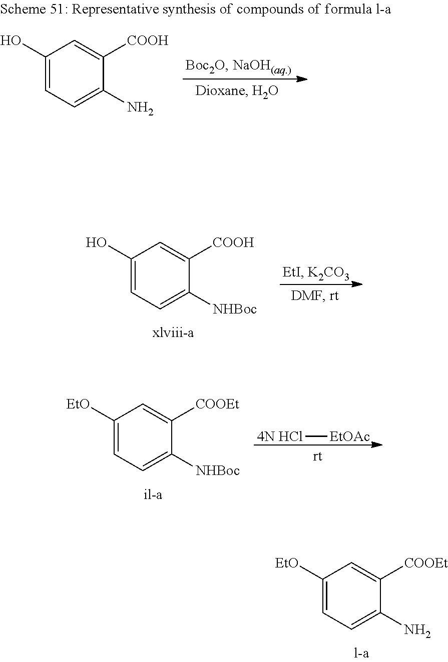

### 2-(tert-butoxycarbonylamino)-5-hydroxybenzoic acid (xlviii-a)

In a 1 litre round bottom flask was cooled 2-amino-5-hydroxybenzoic acid (20 g, 131 mmol) in 1,4-dioxane/water (200 ml/100 ml). A 1N aqueous NaOH solution (200 mL, 200 mmol) was added with stirring, followed by Boc anhydride. The reaction mixture was stirred at room temperature for 1 h and the organics were removed under vacuo. The cooled aqueous solution was acidified with 1N aq. HCl to pH ˜2. A precipitate resulted which was collected by filtration and washed with water and hexane. The resultant product was dried at 50° C. for 24 h to give a grey colored powder which was confirmed to be 2-(tert-butoxycarbonylamino)-5-hydroxybenzoic acid (30 g, 91% yield). ¹H NMR (400 MHz, DMSO) δ 10.06 (s, 1H), 9.44 (s, 1H), 8.04 (d, J=9.0 Hz, 1H), 7.34 (d, J=3.0 Hz, 1H), 6.99 (dd, J=9.0, 3.0 Hz, 1H), 1.48 (s, 9H).

### Ethyl 2-(tert-butoxycarbonylamino)-5-ethoxybenzoate (il-a)

In a 1 litre round bottom flask of 2-(tert-butoxycarbonylamino)-5-hydroxybenzoic acid (78.6 g, 310 mmol) in DMF (500 mL) was added K₂CO₃ (129 g, 931 mmol). Ethyl iodide (74.5 mL, 931 mmol) was added slowly under ice cooling. The reaction mixture was stirred at room temperature for overnight. After the reaction was complete, the mixture was poured into water, and stirred at room temperature for 1-2 h. The resultant precipitate was filtered, washed with water and dried at 60° C. for 24 h to give ethyl 2-(tert-butoxycarbonylamino)-5-ethoxybenzoate (93.9 g, 98% yield) as a brown powder. ¹H NMR (400 MHz, CDCl₃) δ10.00 (s, 1H), 8.33 (d, J=9.2 Hz, 1H), 7.51 (d, J=3.1 Hz, 1H), 7.09 (dd, J=9.3, 3.1 Hz, 1H), 4.37 (q, J=7.1 Hz, 2H), 4.02 (q, J=6.9 Hz, 2H), 1.51 (s, 9H), 1.44-1.37 (m, 6H).

### Ethyl 2-amino-5-ethoxybenzoate (l-a)

To a solution of ethyl 2-(tert-butoxycarbonylamino)-5-ethoxybenzoate (93.9 g, 304 mmol) in ethyl acetate (500 mL) was added a solution 4N HCl in ethyl acetate (304 mL, 1214 mmol) with stirring. The reaction mixture was stirred at 50° C. for 6 h and cooled. The reaction mixture was neutralized to pH 7 by slow addition of NaOH_{(aq.)} and extracted with ethyl acetate. The combined organic layer was washed with water and brine and dried over Na₂SO₄. After filtration and evaporation, the crude product was purified by column chromatography on silica gel (eluted with CH₂Cl₂) to give ethyl 2-amino-5-ethoxybenzoate (57 g, 90% yield) as a pale brown powder. ¹H NMR (400 MHz, CDCl₃) δ 7.38 (d, J=3.0 Hz, 1H), 6.95 (dd, J=8.9, 3.0 Hz, 1H), 6.62 (d, J=8.9 Hz, 1H), 5.39 (s, 2H), 4.33 (q, J=7.1 Hz, 2H), 3.98 (q, J=7.0 Hz, 2H), 1.43-1.35 (m, 6H).

TABLE 12

| | | | | | | ¹H | Puri- | Meth- | | Re- | |
|---|---|---|---|---|---|---|---|---|---|---|---|
| | | | | | | NMR | ty | od of | | ten- | LCMS |
| Num- | Starting | Starting | | Salt | | Sol- | per- | Cou- | | tion | Meth- |
| ber | Material 1 | Material 2 | Product | type | ¹H NMR | vent | cent | pling | LCMS | Time | od |
| 609 | 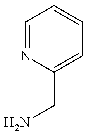 | 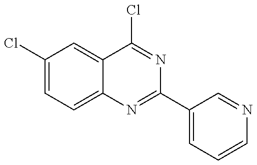 [SMILES: Clc1ccc2nc(-c3cccnc3)nc(Cl)c2c1] | 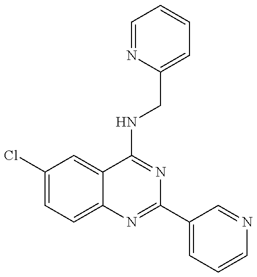 [SMILES: Clc1ccc2nc(-c3cccnc3)nc(NCc3ccccn3)c2c1] | 3HCl | 1H NMR (400 MHz, DMSO) δ 9.90 (d, J = 6.3 Hz, 1H), 9.47 (d, J = 2.0 Hz, 1H), 9.02 (dt, J = 8.0, 1.7, 1.7 Hz, 1H), 8.88 (dd, J = 5.3, 1.6 Hz, 1H), 8.75 (dd, J = 5.5, 1.6 Hz, 1H), 8.68 (t, J = 1.5, 1.5 Hz, 1H), 8.25 (td, J = 7.8, 7.7, 1.7 Hz, 1H), 8.04- 7.83 (m, 4H), 7.70 (dd, J = 7.4, 5.5 Hz, 1H), 5.22 (d, J = 5.4 Hz, 2H).. | DMSO | >98 | G6 | | | |
| 610 | 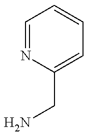 | 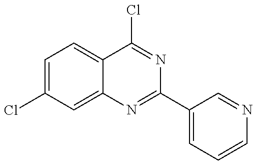 [SMILES: Clc1ccc2c(Cl)nc(-c3cccnc3)nc2c1] | 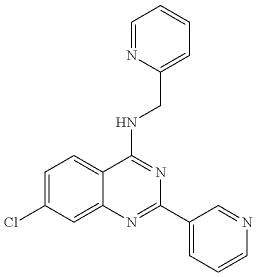 [SMILES: Clc1ccc2c(NCc3ccccn3)nc(-c3cccnc3)nc2c1] | 3HCl | 1H NMR (400 MHz, DMSO) δ 9.96 (s, 1H), 9.48 (d, J = 2.0 Hz, 1H), 9.04 (dd, J = 8.9, 3.0 Hz, 1H), 8.90 (dd, J = 5.2, 1.6 Hz, 1H), 8.76 (d, J = 5.4 Hz, 1H), 8.54 (d, J = 8.9 Hz, 1H), 8.28 (t, J = 8.4, 8.4 Hz, 1H), 8.05-7.87 (m, 3H), 7.84-7.65 (m, 2H), 5.23 (d, J = 5.4 Hz, 2H).. | DMSO | >98 | G6 | | | |
| 611 | 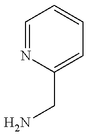 | 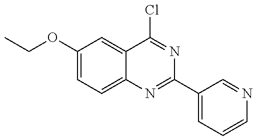 [SMILES: CCOc1ccc2nc(-c3cccnc3)nc(Cl)c2c1] | 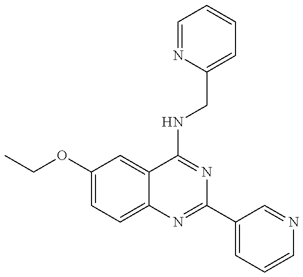 [SMILES: CCOc1ccc2nc(-c3cccnc3)nc(NCc3ccccn3)c2c1] | 3HCl | 1H NMR (400 MHz, DMSO) δ 10.35 (s, 1H), 9.47 (d, J = 2.1 Hz, 1H), 9.03 (d, J = 8.1 Hz, 1H), 8.90 (dd, J = 5.3, 1.6 Hz, 1H), 8.78 (dd, J = 5.5, 1.6 Hz, 1H), 8.30 (t, J = 7.8, 7.8 Hz, 1H), 8.17-7.82 (m, 4H), 7.75 (t, J = 6.5, 6.5 Hz, 1H), 7.63 (dd, J = 9.1, 2.6 Hz, 1H), 5.29 (d, J = 5.5 Hz, 2H), 1.45 (t, J = 6.9, 6.9 Hz, 3H).. | DMSO | >98 | G6 | | | |
| 612 | 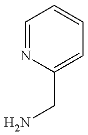 | 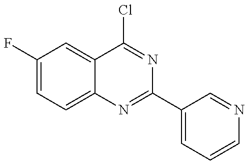 [SMILES: Fc1ccc2nc(-c3cccnc3)nc(Cl)c2c1] | 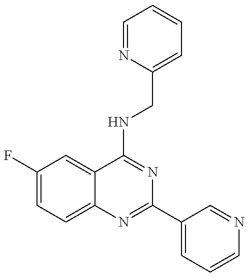 [SMILES: Fc1ccc2nc(-c3cccnc3)nc(NCc3ccccn3)c2c1] | 3HCl | 1H NMR (400 MHz, DMSO) δ 9.80 (s, 1H), 9.46 (d, J = 2.0 Hz, 1H), 8.98 (dt, J = 8.1, 1.8, 1.8 Hz, 1H), 8.86 (dd, J = 5.3, 1.6 Hz, 1H), 8.73 (dd, J = 5.5, 1.6 Hz, 1H), 8.38 (dd, J = 9.6, 2.8 Hz, 1H), 8.19 (t, J = 8.2, .8.2 Hz, 1H), 8.02 (dd, J = 9.2, 5.3 Hz, 1H), 7.99- 7.80 (m, 3H), 7.77-7.56 (m, 1H), 5.19 (d, J = 5.5 Hz, 2H). | DMSO | >98 | G6 | | | |
| 613 | 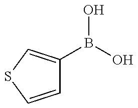 | 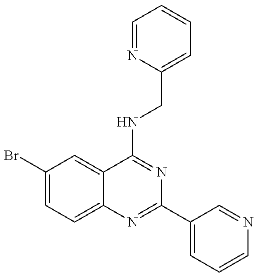 [SMILES: Brc1ccc2nc(-c3cccnc3)nc(NCc3ccccn3)c2c1] | 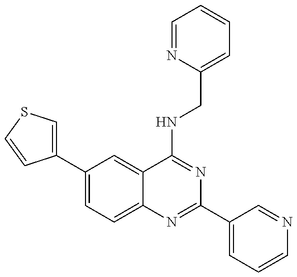 [SMILES: c1ccc(CNc2nc(-c3cccnc3)nc3ccc(-c4ccsc4)cc23)nc1] | 3HCl | 1H NMR (400 MHz, DMSO) δ 10.69 (s, 1H), 9.58-9.42 (m, 1H), 9.25-9.05 (m, 2H), 8.93 (dd, J = 5.3, 1.5 Hz, 1H), 8.81 (dd, J = 5.5, 1.6 Hz, 1H), 8.50- 8.31 (m, 2H), 8.23 (dd, J = 3.0, 1.3 Hz, 1H), 8.09 (dd, J = 8.3, 5.3 Hz, 2H), 8.04-7.86 (m, 2H), 7.86-7.71 (m, 2H), 5.35 (d, J = 5.6 Hz, 2H).. | DMSO | >98 | R4 | | | |
| 614 | 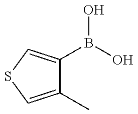 | 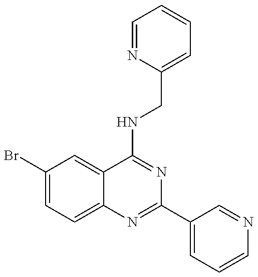 [SMILES: Brc1ccc2nc(-c3cccnc3)nc(NCc3ccccn3)c2c1] | 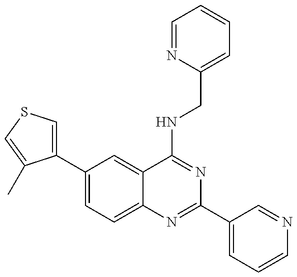 [SMILES: Cc1cscc1-c1ccc2nc(-c3cccnc3)nc(NCc3ccccn3)c2c1] | 3HCl | 1H NMR (400 MHz, DMSO) δ 10.29 (s, 1H), 9.50 (d, J = 2.1 Hz, 1H), 9.05 (d, J = 8.1 Hz, 1H), 8.92 (dd, J = 5.3, 1.6 Hz, 1H), 8.86-8.72 (m, 1H), 8.63 (s, 1H), 8.29 (t, J = 7.8, 7.8 Hz, 1H), 8.16-8.03 (m, 2H), 8.03-7.86 (m, 2H), 7.82-7.67 (m, 2H), 7.40 (dd, J = 3.3, 1.2 Hz, 1H), 5.29 (d, J = 5.5 Hz, 2H), 2.37 (d, J = 1.1 Hz, 3H). | DMSO | >98 | R4 | | | |
| 615 | 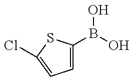 | 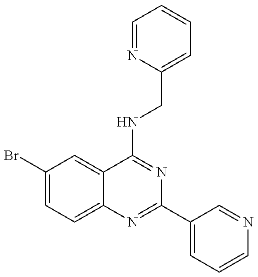 [SMILES: Brc1ccc2nc(-c3cccnc3)nc(NCc3ccccn3)c2c1] | 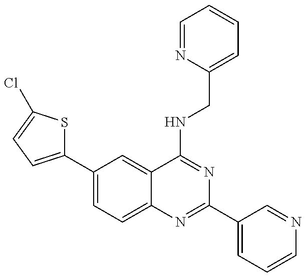 [SMILES: Clc1ccc(-c2ccc3nc(-c4cccnc4)nc(NCc4ccccn4)c3c2)s1] | 3HCl | 1H NMR (400 MHz, DMSO) δ 10.27 (s, 1H), 9.64-9.41 (m, 1H), 9.04 (d, J = 8.2 Hz, 1H), 8.97-8.87 (m, 1H), 8.84 (d, J = 2.1 Hz, 1H), 8.76 (d, J = 5.5 Hz, 1H), 8.39-8.18 (m, 2H), 8.10-7.87 (m, 3H), 7.85- 7.61 (m, 2H), 7.28 (d, J = 4.0 Hz, 1H), 5.28 (d, J = 5.4 Hz, 2H).. | DMSO | >98 | R3 | | | |
| 616 | 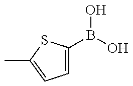 | 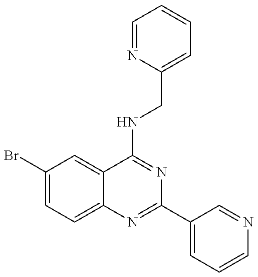 [SMILES: Brc1ccc2nc(-c3cccnc3)nc(NCc3ccccn3)c2c1] | 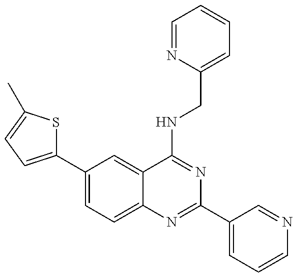 [SMILES: Cc1ccc(-c2ccc3nc(-c4cccnc4)nc(NCc4ccccn4)c3c2)s1] | 3HCl | 1H NMR (400 MHz, DMSO) δ 10.20 (s, 1H), 9.45 (d, J = 2.1 Hz, 1H), 9.08-8.84 (m, 2H), 8.84- 8.65 (m, 2H), 8.18 (t, J = 11.1, 11.1 Hz, 2H), 7.98 (d, J = 8.9 Hz, 1H), 7.84 (t, J = 9.8, 9.8 Hz, 2H), 7.59 (d, J = 3.8 Hz, 1H), 7.03-6.86 (m, 1H), 5.22 (d, J = 5.5 Hz, 2H). | DMSO | >98 | R4 | | | |
| 617 | 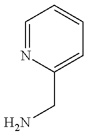 | 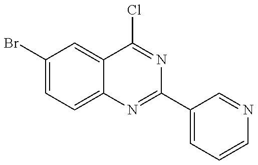 [SMILES: Clc1nc(-c2cccnc2)nc2ccc(Br)cc12] | 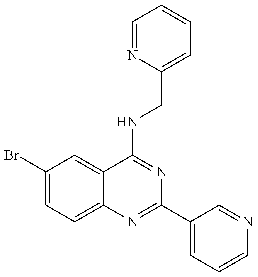 [SMILES: Brc1ccc2nc(-c3cccnc3)nc(NCc3ccccn3)c2c1] | 3HCl | 1H NMR (400 MHz, DMSO) δ 9.43 (dd, J = 2.2, 0.9 Hz, 1H), 9.25 (t, J = 5.7, 5.7 Hz, 1H), 8.77-8.48 (m, 4H), 7.95 (dd, J = 8.9, 2.1 Hz, 1H), 7.84-7.69 (m, 2H), 7.57-7.40 (m, 2H), 7.35-7.12 (m, 1H), 4.97 (d, J = 5.7 Hz, 2H). | DMSO | >98 | G6 | | | |
| 618 | 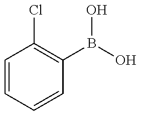 | 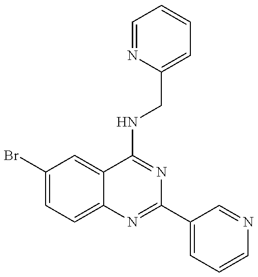 [SMILES: Brc1ccc2nc(-c3cccnc3)nc(NCc3ccccn3)c2c1] | 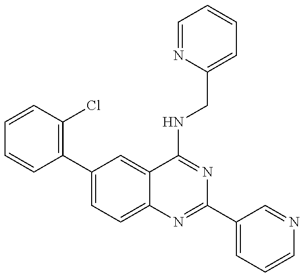 [SMILES: Clc1ccccc1-c1ccc2nc(-c3cccnc3)nc(NCc3ccccn3)c2c1] | 3HCl | 1H NMR (400 MHz, DMSO) δ 10.44 (s, 1H), 9.68-9.48 (m, 1H), 9.24 (d, J = 7.8 Hz, 1H), 9.03-8.66 (m, 3H), 8.41 (d, J = 8.2 Hz, 1H), 8.25-7.91 (m, 4H), 7.91-7.49 (m, 4H), 5.36 (d, J = 5.6 Hz, 1H). | DMSO | >98 | R3 | | | |
| 619 | 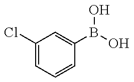 | 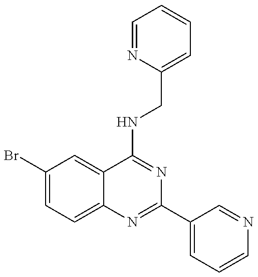 [SMILES: Brc1ccc2nc(-c3cccnc3)nc(NCc3ccccn3)c2c1] | 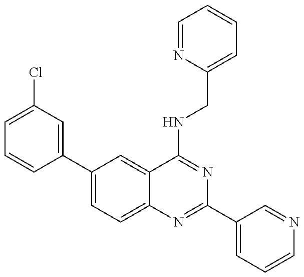 [SMILES: Clc1cccc(-c2ccc3nc(-c4cccnc4)nc(NCc4ccccn4)c3c2)c1] | 3HCl | 1H NMR (400 MHz, DMSO) δ 9.50 (s, 1H), 9.09-8.90 (m, 2H), 8.88 (d, J = 5.0 Hz, 1H), 8.73 (d, J = 5.3 Hz, 1H), 8.37 (d, J = 8.7 Hz, 1H), 8.24-7.99 (m, 3H), 7.97-7.79 (m, 3H), 7.70-7.47 (m, 2H), 5.26 (d, J = 5.3 Hz, 2H), 10.36-10.00 (m, 1H). | DMSO | >98 | R3 | | | |
| 620 | 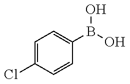 | 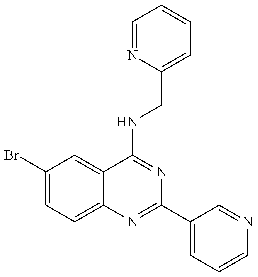 [SMILES: Brc1ccc2nc(-c3cccnc3)nc(NCc3ccccn3)c2c1] | 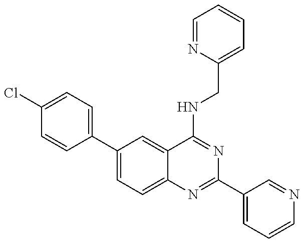 [SMILES: Clc1ccc(-c2ccc3nc(-c4cccnc4)nc(NCc4ccccn4)c3c2)cc1] | 3HCl | 1H NMR (400 MHz, DMSO) δ 10.55 (s, 1H), 9.54 (s, 1H), 9.21-8.96 (m, 2H), 8.93 (d, J = 5.0 Hz, 1H), 8.80 (s, 1H), 8.36 (dd, J = 8.8, 2.0 Hz, 1H), 8.24-7.60 (m, 8H), 5.34 (s, 2H). | DMSO | >98 | R3 | | | |
| 621 | 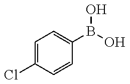 | 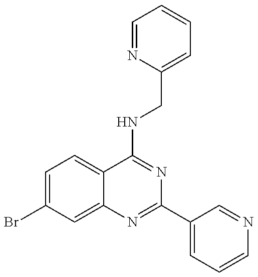 [SMILES: Brc1ccc2c(NCc3ccccn3)nc(-c3cccnc3)nc2c1] | 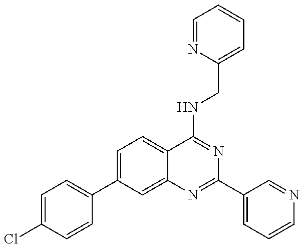 [SMILES: Clc1ccc(-c2ccc3c(NCc4ccccn4)nc(-c4cccnc4)nc3c2)cc1] | 3HCl | 1H NMR (400 MHz, DMSO) δ 10.23 (s, 1H), 9.51 (s, 1H), 9.04 (s, 1H), 8.90 (d, J = 5.1 Hz, 1H), 8.75 (d, J = 5.3 Hz, 1H), 8.65 (d, J = 8.8 Hz, 1H), 8.36-8.18 (m, 2H), 8.13- 8.06 (m, 1H), 7.97-7.85 (m, 3H), 7.74-7.60 (m, 2H), 5.27 (d, J = 5.6 Hz, 2H). | DMSO | >98 | R3 | | | |
| 622 | 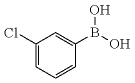 | 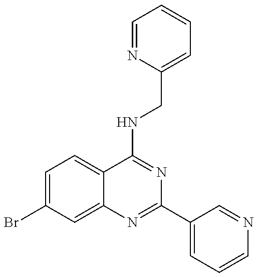 [SMILES: Brc1ccc2c(NCc3ccccn3)nc(-c3cccnc3)nc2c1] | 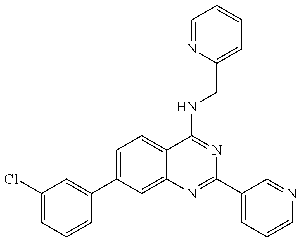 [SMILES: Clc1cccc(-c2ccc3c(NCc4ccccn4)nc(-c4cccnc4)nc3c2)c1] | 3HCl | 1H NMR (400 MHz, DMSO) δ 9.61-9.38 (m, 2H), 8.80-8.68 (m, 2H), 8.65-8.48 (m, 2H), 8.15 (d, J = 1.9 Hz, 1H), 8.06-7.93 (m, 2H), 7.88-7.79 (m, 2H), 7.66-7.50 (m, 4H), 7.41-7.29 (m, 1H), 5.07 (d, J = 5.8 Hz, 2H). | DMSO | >98 | R3 | | | |
| 623 | 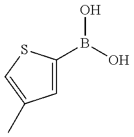 | 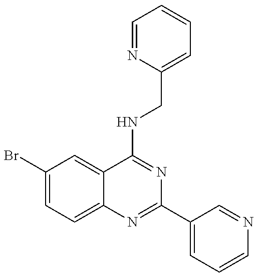 [SMILES: Brc1ccc2nc(-c3cccnc3)nc(NCc3ccccn3)c2c1] | 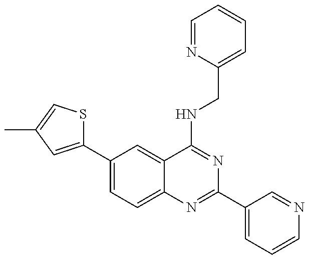 [SMILES: Cc1csc(-c2ccc3nc(-c4cccnc4)nc(NCc4ccccn4)c3c2)c1] | 3HCl | 1H NMR (400 MHz, DMSO) δ 10.23 (s, 1H), 9.47 (dd, J = 2.2, 0.7 Hz, 1H), 8.98 (d, J = 8.1 Hz, 1H), 8.88 (dd, J = 5.2, 1.6 Hz, 1H), 8.82 (d, J = 2.1 Hz, 1H), 8.73 (d, J = 5.6 Hz, 1H), 8.28-8.15 (m, 1H), 8.01 (d, J = 8.7 Hz, 1H), 7.97-7.82 (m, 2H), 7.74-7.58 (m, 2H), 7.28 (t, J = 1.2, 1.2 Hz, 1H), 5.25 (d, J = 5.5 Hz, 2H), 2.30 (d, J = 1.2 Hz, 3H). | DMSO | >98 | R4 | | | |
| 624 | 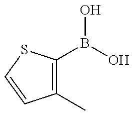 | 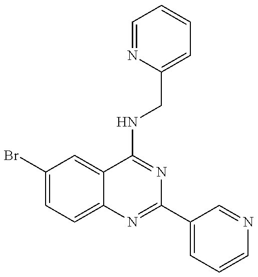 [SMILES: Brc1ccc2nc(-c3cccnc3)nc(NCc3ccccn3)c2c1] | 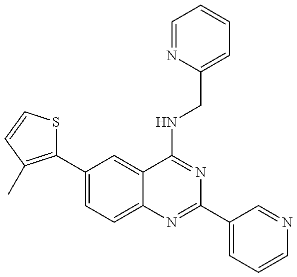 [SMILES: Cc1ccsc1-c1ccc2nc(-c3cccnc3)nc(NCc3ccccn3)c2c1] | 3HCl | 1H NMR (400 MHz, DMSO) δ 10.40 (s, 1H), 9.53 (d, J = 2.0 Hz, 1H), 9.14 (d, J = 8.0 Hz, 1H), 8.93 (dd, J = 5.3, 1.5 Hz, 1H), 8.84-8.74 (m, 1H), 8.65 (d, J = 1.7 Hz, 1H), 8.35 (t, J = 7.8, 7.8 Hz, 1H), 8.20-8.00 (m, 3H), 8.00-7.90 (m, 1H), 7.85-7.73 (m, 1H), 7.62 (d, J = 5.1 Hz, 1H), 7.10 (d, J = 5.1 Hz, 1H), 5.33 (d, J = 5.5 Hz, 2H), 2.43 (s, 3H). | DMSO | >98 | R4 | | | |
| 625 | 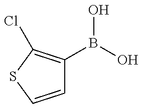 | 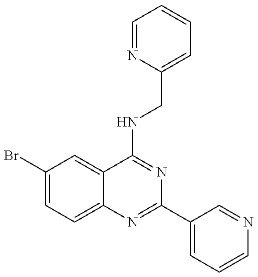 [SMILES: Brc1ccc2nc(-c3cccnc3)nc(NCc3ccccn3)c2c1] | 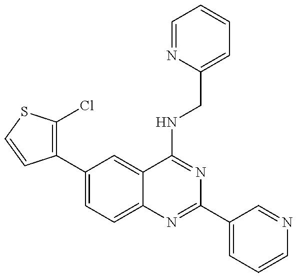 [SMILES: Clc1sccc1-c1ccc2nc(-c3cccnc3)nc(NCc3ccccn3)c2c1] | 3HCl | 11H NMR (400 MHz, DMSO) δ 10.29 (s, 1H), 9.52 (s, 1H), 9.23- 8.67 (m, 3H), 8.36-7.81 (m, 4H), 7.81-7.39 (m, 4H), 5.28 (s, 2H). | DMSO | >98 | R3 | | | |
| 626 | 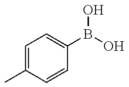 | 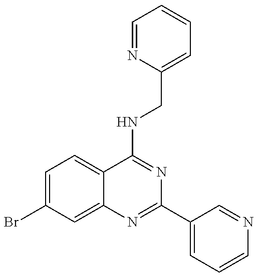 [SMILES: Brc1ccc2c(NCc3ccccn3)nc(-c3cccnc3)nc2c1] | 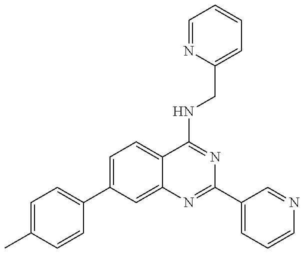 [SMILES: Cc1ccc(-c2ccc3c(NCc4ccccn4)nc(-c4cccnc4)nc3c2)cc1] | 3HCl | 1H NMR (400 MHz, DMSO) δ 10.24 (s, 1H), 9.48 (d, J = 2.1 Hz, 1H), 9.08-8.85 (m, 2H), 8.71 (d, J = 5.2 Hz, 1H), 8.62 (d, J = 8.6 Hz, 1H), 8.29 (s, 1H), 8.10 (t, J = 10.4, 10.4 Hz, 2H), 7.97-7.73 (m, 4H), 7.60 (s, 1H), 7.50-7.34 (m, 2H), 5.23 (d, J = 5.5 Hz, 2H), 2.40 (s, 3H). | DMSO | >98 | R4 | | | |
| 627 | 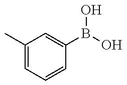 | 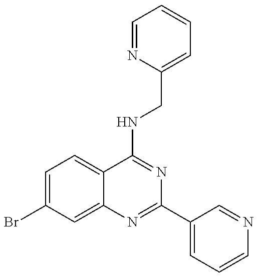 [SMILES: Brc1ccc2c(NCc3ccccn3)nc(-c3cccnc3)nc2c1] | 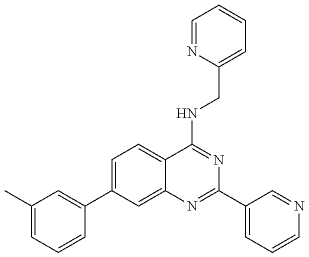 [SMILES: Cc1cccc(-c2ccc3c(NCc4ccccn4)nc(-c4cccnc4)nc3c2)c1] | 3HCl | 1H NMR (400 MHz, DMSO) δ 10.53 (s, 1H), 9.55 (d, J = 2.0 Hz, 1H), 9.15 (d, J = 8.1 Hz, 1H), 8.94 (dd, J = 5.3, 1.5 Hz, 1H), 8.81 (dd, J = 5.8, 1.6 Hz, 1H), 8.71 (d, J = 8.7 Hz, 1H), 8.44-8.31 (m, 2H), 8.17-7.99 (m, 2H), 7.94 (dd, J = 8.2, 5.3 Hz, 1H), 7.80 (t, J = 6.6, 6.6 Hz, 1H), 7.74-7.64 (m, 2H), 7.47 (t, J = 7.6, 7.6 Hz, 1H), 7.39-7.24 (m, 1H), 5.35 (d, J = 5.4 Hz, 2H), 2.44 (s, 3H). | DMSO | >98 | R4 | | | |
| 628 | 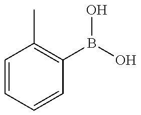 | 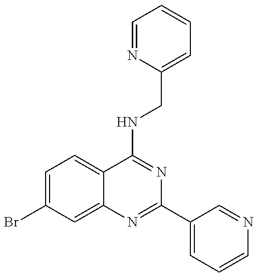 [SMILES: Brc1ccc2c(NCc3ccccn3)nc(-c3cccnc3)nc2c1] | 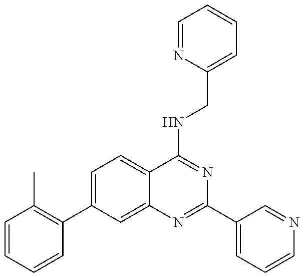 [SMILES: Cc1ccccc1-c1ccc2c(NCc3ccccn3)nc(-c3cccnc3)nc2c1] | 3HCl | 1H NMR (400 MHz, DMSO) δ 10.47 (s, 1H), 9.52 (dd, J = 1.9, 0.8 Hz, 1H), 9.07 (d, J = 8.0 Hz, 1H), 8.92 (dd, J = 5.4, 1.5 Hz, 1H), 8.78 (dd, J = 5.1, 1.2 Hz, 1H), 8.67 (d, J = 8.5 Hz, 1H), 8.38-8.24 (m, 1H), 8.08 (s, 1H), 7.98 (d, J = 8.2 Hz, 1H), 7.89 (dd, J = 8.0, 5.2 Hz, 1H), 7.84-7.68 (m, 2H), 7.44-7.31 (m, 4H), 5.32 (d, J = 5.5 Hz, 2H), 2.33 (s, 3H). | DMSO | >98 | R4 | | | |
| 629 | 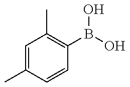 | 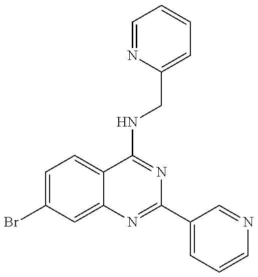 [SMILES: Brc1ccc2c(NCc3ccccn3)nc(-c3cccnc3)nc2c1] | 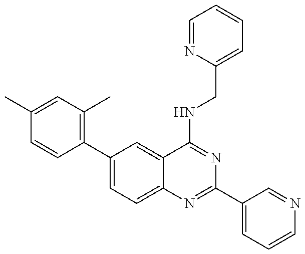 [SMILES: Cc1ccc(-c2ccc3nc(-c4cccnc4)nc(NCc4ccccn4)c3c2)c(C)c1] | 3HCl | 1H NMR (400 MHz, DMSO) δ 9.45 (s, 1H), 8.98-8.76 (m, 2H), 8.76- 8.62 (m, 1H), 8.58-8.40 (m, 1H), 8.17-7.91 (m, 3H), 7.88-7.64 (m, 2H), 7.64-7.44 (m, 1H), 7.32-7.09 (m, 2H), 5.16 (d, J = 6.7 Hz, 2H), 2.36 (s, 2H), 2.31 (s, 2H), 10.28-9.87 (m, 1H). | DMSO | >98 | R4 | | | |
| 630 | 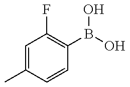 | 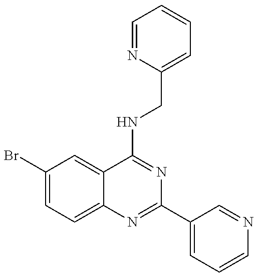 [SMILES: Brc1ccc2nc(-c3cccnc3)nc(NCc3ccccn3)c2c1] | 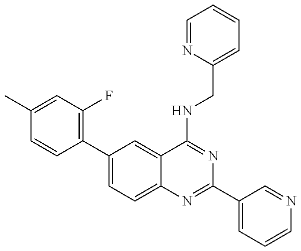 [SMILES: Cc1ccc(-c2ccc3nc(-c4cccnc4)nc(NCc4ccccn4)c3c2)c(F)c1] | 3HCl | 1H NMR (400 MHz, DMSO) δ 9.45 (s, 1H), 8.98-8.76 (m, 2H), 8.76- 8.62 (m, 1H), 8.58-8.40 (m, 1H), 8.17-7.91 (m, 3H), 7.88-7.64 (m, 2H), 7.64-7.44 (m, 1H), 7.32-7.09 (m, 2H), 5.16 (d, J = 6.7 Hz, 2H), 2.36 (s, 2H), 2.31 (s, 2H), 10.28-9.87 (m, 1H). | DMSO | >98 | R4 | | | |
| 631 | 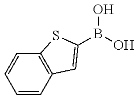 [SMILES: OB(O)c1cc2ccccc2s1] | 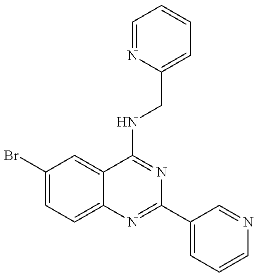 [SMILES: Brc1ccc2nc(-c3cccnc3)nc(NCc3ccccn3)c2c1] | 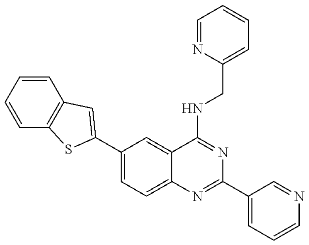 [SMILES: c1ccc(CNc2nc(-c3cccnc3)nc3ccc(-c4cc5ccccc5s4)cc23)nc1] | 3HCl | 1H NMR (400 MHz, DMSO) δ 10.42 (s, 1H), 9.55 (d, J = 2.0 Hz, 1H), 9.24-9.06 (m, 1H), 8.94 (dd, J = 5.3, 1.5 Hz, 1H), 8.90-8.75 (m, 2H), 8.37 (td, J = 7.7, 7.6, 1.7 Hz, 1H), 8.20-8.11 (m, 2H), 8.05 (d, J = 8.0 Hz, 1H), 7.96 (dd, J = 8.2, 5.3 Hz, 1H), 7.85-7.73 (m, 1H), 7.73-7.61 (m, 1H), 7.35-7.14 (m, 2H), 5.34 (d, J = 5.6 Hz, 2H), 2.41 (s, 3H). | DMSO | >98 | R4 | | | |
| 632 | 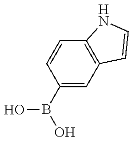 [SMILES: OB(O)c1ccc2[nH]ccc2c1] | 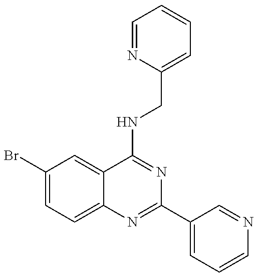 [SMILES: Brc1ccc2nc(-c3cccnc3)nc(NCc3ccccn3)c2c1] | 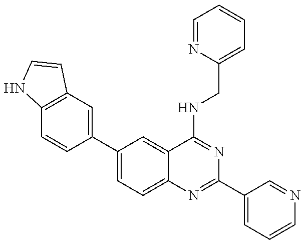 [SMILES: c1ccc(CNc2nc(-c3cccnc3)nc3ccc(-c4ccc5[nH]ccc5c4)cc23)nc1] | 3HCl | 1H NMR (400 MHz, DMSO) δ 10.16 (s, 1H), 9.48 (s, 1H), 9.03-8.81 (m, 3H), 8.73 (d, J = 5.5 Hz, 1H), 8.37 (dd, J = 8.6, 1.9 Hz, 1H), 8.28-7.96 (m, 4H), 7.96-7.79 (m, 3H), 7.63 (s, 1H), 7.51-7.37 (m, 2H), 5.25 (t, J = 4.6, 4.6 Hz, 2H). | DMSO | >98 | R4 | | | |
| 633 | 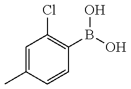 | 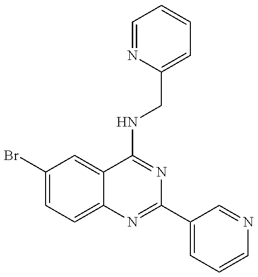 [SMILES: Brc1ccc2nc(-c3cccnc3)nc(NCc3ccccn3)c2c1] | 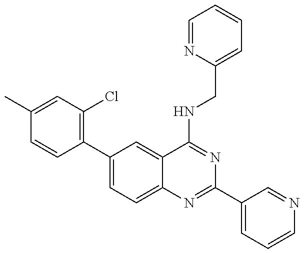 [SMILES: Cc1ccc(-c2ccc3nc(-c4cccnc4)nc(NCc4ccccn4)c3c2)c(Cl)c1] | 3HCl | 1H NMR (400 MHz, DMSO) δ 10.28 (s, 1H), 9.62-9.49 (m, 1H), 9.12 (d, J = 7.8 Hz, 1H), 8.93 (dd, J = 5.3, 1.5 Hz, 1H), 8.78 (d, J = 5.5 Hz, 1H), 8.64 (s, 1H), 8.31 (t, J = 7.8, 7.8 Hz, 1H), 8.19-8.03 (m, 2H), 8.03-7.89 (m, 2H), 7.76 (t, J = 6.6, 6.6 Hz, 1H), 7.55-7.44 (m, 2H), 7.41-7.31 (m, 1H), 5.30 (d, J = 5.4 Hz, 2H). | DMSO | >98 | R3 | | | |
| 634 | 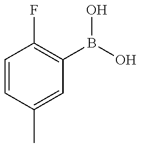 | 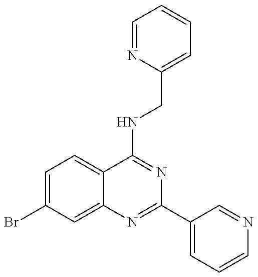 [SMILES: Brc1ccc2c(NCc3ccccn3)nc(-c3cccnc3)nc2c1] | 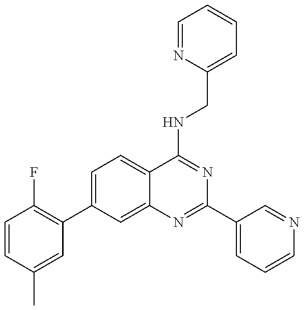 [SMILES: Cc1ccc(F)c(-c2ccc3c(NCc4ccccn4)nc(-c4cccnc4)nc3c2)c1] | 3HCl | 1H NMR (400 MHz, DMSO) δ 9.46 (s, 1H), 8.96-8.76 (m, 2H), 8.76- 8.52 (m, 2H), 8.15-7.69 (m, 5H), 7.61-7.48 (m, 2H), 7.39-7.21 (m, 2H), 5.19 (s , 2H), 2.40 (s, 3H), 10.20-9.76 (m, 1H). | DMSO | >98 | R4 | | | |
| 635 | 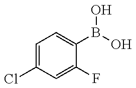 [SMILES: OB(O)c1ccc(Cl)cc1F] | 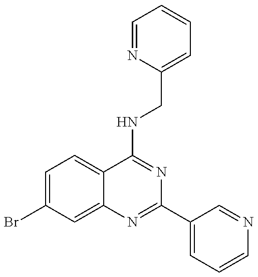 [SMILES: Brc1ccc2c(NCc3ccccn3)nc(-c3cccnc3)nc2c1] | 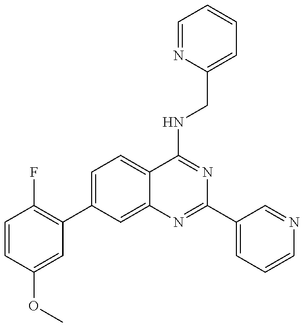 [SMILES: COc1ccc(F)c(-c2ccc3c(NCc4ccccn4)nc(-c4cccnc4)nc3c2)c1] | 3HCl | 1H NMR (400 MHz, DMSO) δ 10.11 (s, 1H), 9.48 (s, 1H), 9.07-8.81 (m, 2H), 8.78-8.56 (m, 2H), 8.16 (s, 1H), 8.01-7.71 (m, 3H), 7.60 (s, 1H), 7.36 (dd, J = 10.4, 9.0 Hz, 1H), 7.24 (dd, J = 6.4, 3.1 Hz, 1H), 7.09 (dt, J = 9.1, 3.6, 3.6 Hz, 1H), 5.22 (s, 2H), 3.85 (s, 3H). | DMSO | >98 | R4 | | | |
| 636 | 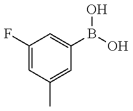 | 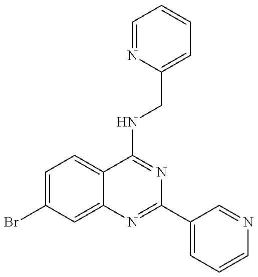 [SMILES: Brc1ccc2c(NCc3ccccn3)nc(-c3cccnc3)nc2c1] | 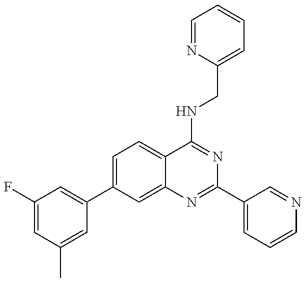 [SMILES: Cc1cc(F)cc(-c2ccc3c(NCc4ccccn4)nc(-c4cccnc4)nc3c2)c1] | 3HCl | 1H NMR (400 MHz, DMSO) δ 10.11 (s, 1H), 9.48 (s, 1H), 9.07-8.81 (m, 2H), 8.78-8.56 (m, 2H), 8.16 (s, 1H), 8.01-7.71 (m, 3H), 7.60 (s, 1H), 7.36 (dd, J = 10.4, 9.0 Hz, 1H), 7.24 (dd, J = 6.4, 3.1 Hz, 1H), 7.09 (dt, J = 9.1, 3.6, 3.6 Hz, 1H), 5.22 (s, 2H), 3.85 (s, 3H). | DMSO | >98 | R4 | | | |
| 637 | 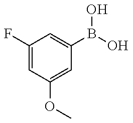 | 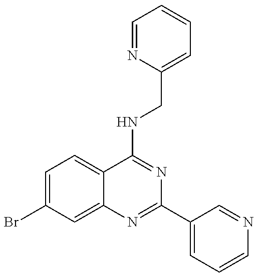 [SMILES: Brc1ccc2c(NCc3ccccn3)nc(-c3cccnc3)nc2c1] | 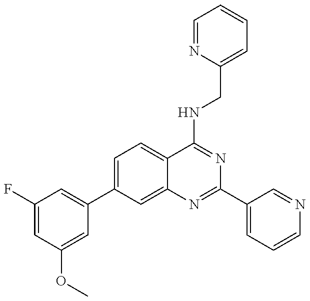 [SMILES: COc1cc(F)cc(-c2ccc3c(NCc4ccccn4)nc(-c4cccnc4)nc3c2)c1] | 3HCl | 1H NMR (400 MHz, DMSO) δ 9.46 (d, J = 2.0 Hz, 1H), 8.83 (d, J = 5.2 Hz, 1H), 8.67 (d, J = 5.1 Hz, 1H), 8.57 (d, J = 8.7 Hz, 1H), 8.22 (s, 1H), 8.07 (d, J = 9.1 Hz, 1H), 7.75 (s, 2H), 7.52 (s, 1H), 7.37-7.23 (m, 2H), 7.05-6.91 (m, 1H), 5.16 (d, J = 5.6 Hz, 2H), 3.90 (s, 3H), 10.03-9.71 (m, 1H). | DMSO | >98 | R4 | | | |
| 638 | 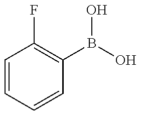 | 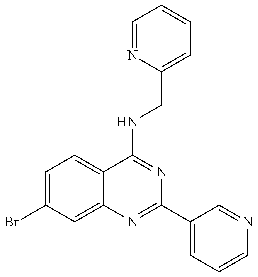 [SMILES: Brc1ccc2c(NCc3ccccn3)nc(-c3cccnc3)nc2c1] | 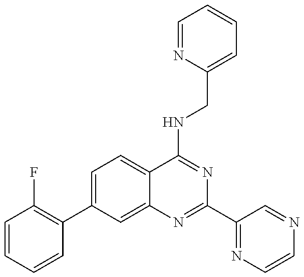 [SMILES: Fc1ccccc1-c1ccc2c(NCc3ccccn3)nc(-c3cnccn3)nc2c1] | 2HCl | 1H NMR (400 MHz, DMSO) δ 9.66 (d, J = 1.5 Hz, 1H), 9.06-8.93 (m, 2H), 8.76 (d, J = 8.6 Hz, 1H), 8.62 (d, J = 4.8 Hz, 1H), 8.48 (s, 1H), 8.07 (d, J = 8.6 Hz, 1H), 7.92 (s, 1H), 7.83-7.66 (m, 2H), 7.59 (t, J = 6.6, 6.6 Hz, 1H), 7.54-7.35 (m, 3H), 5.29 (d, J = 5.4 Hz, 2H), 11.21-10.88 (m, 2H). | DMSO | >98 | R4 | | | |
| 639 | 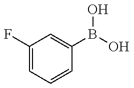 | 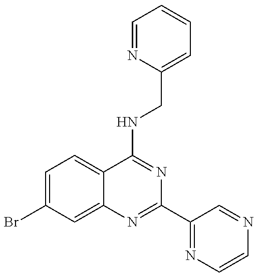 [SMILES: Brc1ccc2c(NCc3ccccn3)nc(-c3cnccn3)nc2c1] | 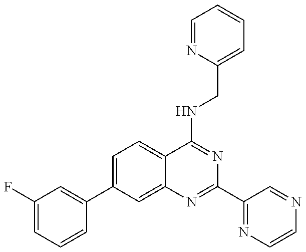 [SMILES: Fc1cccc(-c2ccc3c(NCc4ccccn4)nc(-c4cnccn4)nc3c2)c1] | 2HCl | 1H NMR (400 MHz, DMSO) δ 10.72 (s, 1H), 9.60 (d, J = 1.4 Hz, 1H), 9.02-8.89 (m, 2H), 8.73 (d, J = 8.6 Hz, 1H), 8.60 (ddd, J = 4.8, 1.8, 0.9 Hz, 1H), 8.48 (s, 1H), 8.19 (dd, J = 8.7, 1.9 Hz, 1H), 7.86 (td, J = 7.7, 7.7, 1.8 Hz, 1H), 7.81-7.62 (m, 4H), 7.37 (ddd, J = 7.7, 5.3, 2.6 Hz, 2H), 5.21 (d, J = 5.6 Hz, 2H). | DMSO | >98 | R4 | | | |
| 640 | 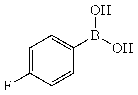 | 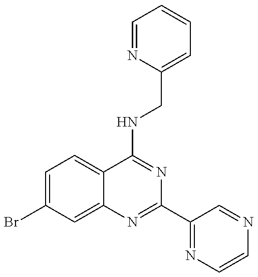 [SMILES: Brc1ccc2c(NCc3ccccn3)nc(-c3cnccn3)nc2c1] | 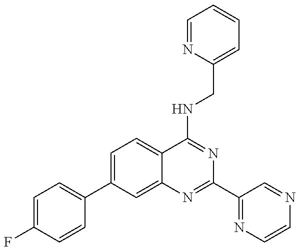 [SMILES: Fc1ccc(-c2ccc3c(NCc4ccccn4)nc(-c4cnccn4)nc3c2)cc1] | 2HCl | 1H NMR (400 MHz, DMSO) δ 11.16 (s, 1H), 9.67 (d, J = 1.4 Hz, 1H), 9.05-8.97 (m, 2H), 8.78 (d, J = 8.6 Hz, 1H), 8.65 (d, J = 4.9 Hz, 1H), 8.54 (d, J = 1.8 Hz, 1H), 8.22 (dd, J = 8.6, 1.8 Hz, 1H), 8.02-7.85 (m, 3H), 7.75 (d, J = 7.8 Hz, 1H), 7.57-7.39 (m, 3H), 5.31 (d, J = 5.6 Hz, 2H). | DMSO | >98 | R4 | | | |
| 641 | 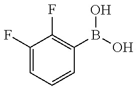 [SMILES: OB(O)c1cccc(F)c1F] | 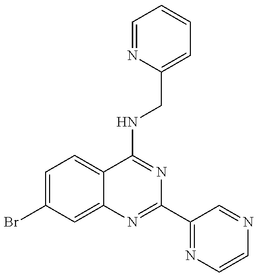 [SMILES: Brc1ccc2c(NCc3ccccn3)nc(-c3cnccn3)nc2c1] | 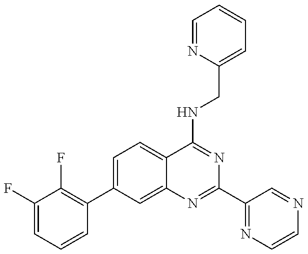 [SMILES: Fc1cccc(-c2ccc3c(NCc4ccccn4)nc(-c4cnccn4)nc3c2)c1F] | 2HCl | 1H NMR (400 MHz, DMSO) δ 9.65 (s, 1H), 9.15-8.92 (m, 2H), 8.76 (d, J = 8.8 Hz, 1H), 8.62 (d, J = 4.9 Hz, 1H), 8.47 (s, 1H), 11.22-10.70 (m, 3H), 8.07 (d, J = 8.5 Hz, 1H), 7.91 (s, 1H), 7.78- 7.36 (m, 5H), 5.27 (d, J = 5.5 Hz, 2H). | DMSO | >98 | R4 | | | |
| 642 | 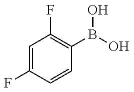 [SMILES: OB(O)c1ccc(F)cc1F] | 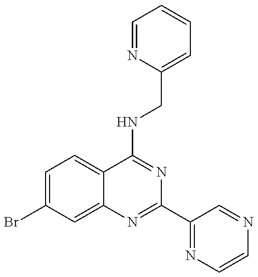 [SMILES: Brc1ccc2c(NCc3ccccn3)nc(-c3cnccn3)nc2c1] | 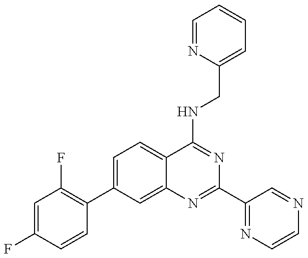 [SMILES: Fc1ccc(-c2ccc3c(NCc4ccccn4)nc(-c4cnccn4)nc3c2)c(F)c1] | 2HCl | 1H NMR (400 MHz, DMSO) δ 11.24 (s, 1H), 9.67 (d, J = 1.5 Hz, 1H), 9.10-8.94 (m, 2H), 8.82 (d, J = 8.7 Hz, 1H), 8.67 (d, J = 5.0 Hz, 1H), 8.48 (s, 1H), 8.13-7.93 (m, 2H), 7.93-7.73 (m, 2H), 7.62-7.42 (m, 2H), 7.42-7.26 (m, 1H), 5.33 (d, J = 5.6 Hz, 2H). | DMSO | >98 | R4 | | | |
| 643 | 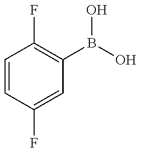 [SMILES: OB(O)c1cc(F)ccc1F] | 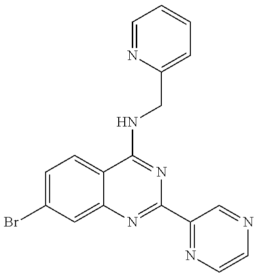 [SMILES: Brc1ccc2c(NCc3ccccn3)nc(-c3cnccn3)nc2c1] | 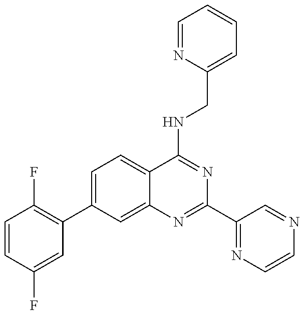 [SMILES: Fc1ccc(F)c(-c2ccc3c(NCc4ccccn4)nc(-c4cnccn4)nc3c2)c1] | 2HCl | 1H NMR (400 MHz, DMSO) δ 11.14 (s, 1H), 9.66 (d, J = 1.4 Hz, 1H), 9.07-8.93 (m, 2H), 8.81 (d, J = 8.7 Hz, 1H), 8.65 (d, J = 4.9 Hz, 1H), 8.51 (s, 1H), 8.09 (d, J = 8.6 Hz, 1H), 7.98 (s, 1H), 7.81-7.61 (m, 2H), 7.61-7.34 (m, 3H), 5.31 (d, J = 5.6 Hz, 2H). | DMSO | >98 | R4 | | | |
| 644 | 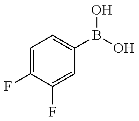 [SMILES: OB(O)c1ccc(F)c(F)c1] | 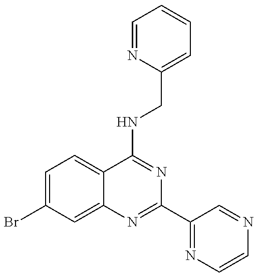 [SMILES: Brc1ccc2c(NCc3ccccn3)nc(-c3cnccn3)nc2c1] | 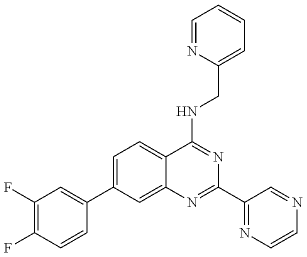 [SMILES: Fc1ccc(-c2ccc3c(NCc4ccccn4)nc(-c4cnccn4)nc3c2)cc1F] | 2HCl | 1H NMR (400 MHz, DMSO) δ 10.85 (s, 1H), 9.62 (d, J = 1.4 Hz, 1H), 9.05-8.91 (m, 2H), 8.80-8.68 (m, 2H), 8.48 (s, 1H), 8.19 (dd, J = 8.7, 1.8 Hz, 1H), 8.06-7.95 (m, 1H), 7.89 (t, J = 7.7, 7.7 Hz, 1H), 7.79-7.60 (m, 3H), 7.51 (d, J = 8.7 Hz, 1H), 7.40 (t, J = 6.2, 6.2 Hz, 1H), 5.24 (d, J = 5.6 Hz, 2H). | DMSO | >98 | R4 | | | |
| 645 | 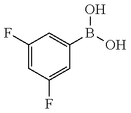 [SMILES: OB(O)c1cc(F)cc(F)c1] | 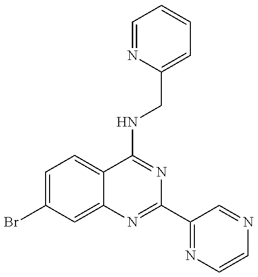 [SMILES: Brc1ccc2c(NCc3ccccn3)nc(-c3cnccn3)nc2c1] | 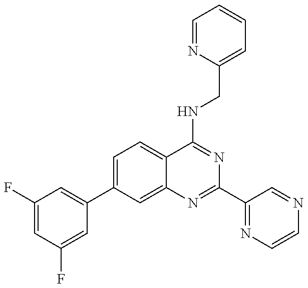 [SMILES: Fc1cc(F)cc(-c2ccc3c(NCc4ccccn4)nc(-c4cnccn4)nc3c2)c1] | 2HCl | 1H NMR (400 MHz, DMSO) δ 11.26 (s, 1H), 9.66 (d, J = 1.4 Hz, 1H), 9.08-8.97 (m, 2H), 8.85 (d, J = 8.8 Hz, 1H), 8.69 (d, J = 5.0 Hz, 1H), 8.58 (d, J = 1.8 Hz, 1H), 8.27 (dd, J = 8.7, 1.8 Hz, 1H), 8.06 (s, 1H), 7.82 (d, J = 8.1 Hz, 1H), 7.64 (dd, J = 8.7, 2.3 Hz, 2H), 7.55 (s, 1H), 7.46 (tt, J = 9.3, 9.3, 2.3, 2.3 Hz, 1H), 5.34 (d, J = 5.5 Hz, 2H). | DMSO | >98 | R4 | | | |

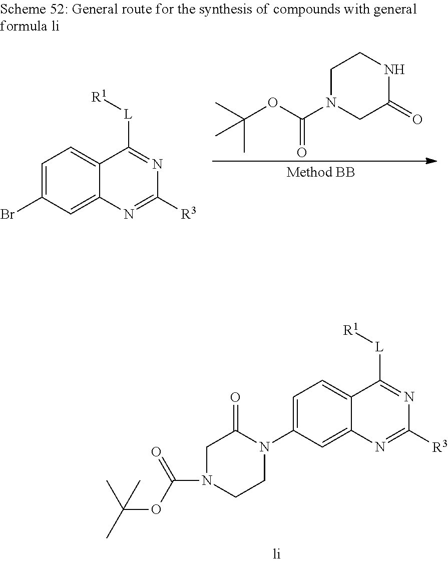

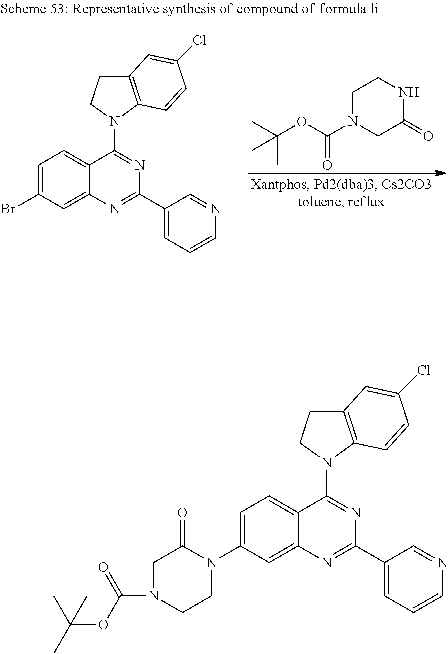 [SMILES: Clc1ccc2c(c1)CCN2c1nc(-c2cccnc2)nc2cc(Br)ccc12 ; CC(C)(C)OC(=O)N1CCNC(=O)C1 ; CC(C)(C)OC(=O)N1CCN(c2ccc3c(N4CCc5cc(Cl)ccc54)nc(-c4cccnc4)nc3c2)C(=O)C1]

### Method BB

A mixture of Reactant 1 (0.2 g, 0.457 mmol), 4-N-Boc-2-oxo-piper (0.137 g, 0.685 mmol), XANTPHOS (0.026 g, 0.046 mmol), Pd2(dba)₃ (0.042 g mmol) and Cs2CO3 (0.208 g, 0.640 mmol) in toluene (10 ml) was refluxed for 1 the reaction mixture was added AcOEt and washed with H2O and brine. Dried over Na2SO4 and AcOEt was removed under reduced pressure to give crude solid which was purified with NH—SiO2-column chromatograghy (Hex:AcOEt=5:1-1:1) to give yellow amorphous (0.22 g).

¹H NMR (400 MHz, CDCl₃) δ 1.55-1.51 (m, OH), 1.64 (s, 91-1), 3.25 (t, J=7.9 Hz, 2H), 4.01-3.82 (m, 4H), 4.36-4.27 (m, 2H), 4.56 (t, J=8.0 Hz, 2H), 7.17 (dd, J=8.6, 2.3 Hz, 1H), 7.48-7.29 (m, 31-1), 7.62 (d, J=9.1 Hz, 1H), 7.84 (d, J=2.2 Hz, 1H), 8.04 (d, J=9.0 Hz, 1H), 8.82-8.67 (m, 2H), 9.76-9.67 (m, 1H).

TABLE 13

| | | | | | | | | Method | | Reten- | |
|---|---|---|---|---|---|---|---|---|---|---|---|
| | Starting | Starting | | Salt | | ¹H NMR | Purity | of | | tion | LCMS |
| Number | Material 1 | Material 2 | Product | type | ¹H NMR | Solvent | percent | Coupling | LCMS | Time | Method |
| 646 | 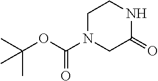 | 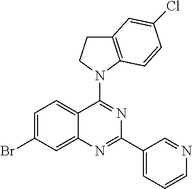 [SMILES: Clc1ccc2c(c1)CCN2c1nc(-c2cccnc2)nc2cc(Br)ccc12] | 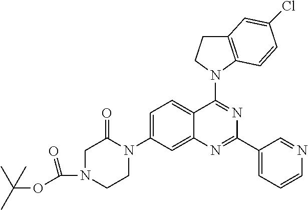 [SMILES: CC(C)(C)OC(=O)N1CCN(c2ccc3c(N4CCc5cc(Cl)ccc54)nc(-c4cccnc4)nc3c2)C(=O)C1] | | 1H NMR (400 MHz, CDCl3) δ 1.55-1.51 (m, 0H), 1.64 (s, 9H), 3.25 (t, J = 7.9 Hz, 2H), 4.01-3.82 (m, 4H), 4.36-4.27 (m, 2H), 4.56 (t, J = 8.0 Hz, 2H), 7.17 (dd, J = 8.6, 2.3 Hz, 1H), 7.48-7.29 (m, 3H), 7.62 (d, J = 9.1 Hz, 1H), 7.84 (d, J = 2.2 Hz, 1H), | CDCl3 | >98 | BB | | | |
| | | | | | 8.04 (d, J = 9.0 Hz, | | | | | | |
| | | | | | 1H), 8.82-8.67 (m, | | | | | | |
| | | | | | 2H), 9.76-9.67 (m, | | | | | | |
| | | | | | 1H). | | | | | | |
| 647 | 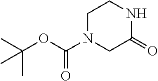 | 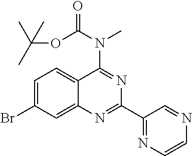 [SMILES: CN(C(=O)OC(C)(C)C)c1nc(-c2cnccn2)nc2cc(Br)ccc12] | 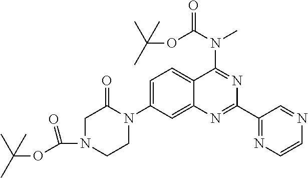 [SMILES: CN(C(=O)OC(C)(C)C)c1nc(-c2cnccn2)nc2cc(N3CCN(C(=O)OC(C)(C)C)CC3=O)ccc12] | | 1H NMR (400 MHz, CDCl3) δ 1.40 (s, 9H), 1.55 (dt, J = 5.7, 2.7 Hz, 9H), 3.87 (q, J = 5.2, 4.7 Hz, 2H), 3.94 (dt, J = 5.9, 4.3 Hz, 2H), 4.33 (s, 2H), 7.98-7.86 (m, 2H), 8.05 (d, J = 2.1 Hz, 1H), 8.72 (d, J = | CDCl3 | >98 | BB | | | |
| | | | | | 2.4 Hz, 1H), 8.90- | | | | | | |
| | | | | | 8.80 (m, 1H), 9.84 | | | | | | |
| | | | | | (d, J = 1.4 Hz, 1H). | | | | | | |

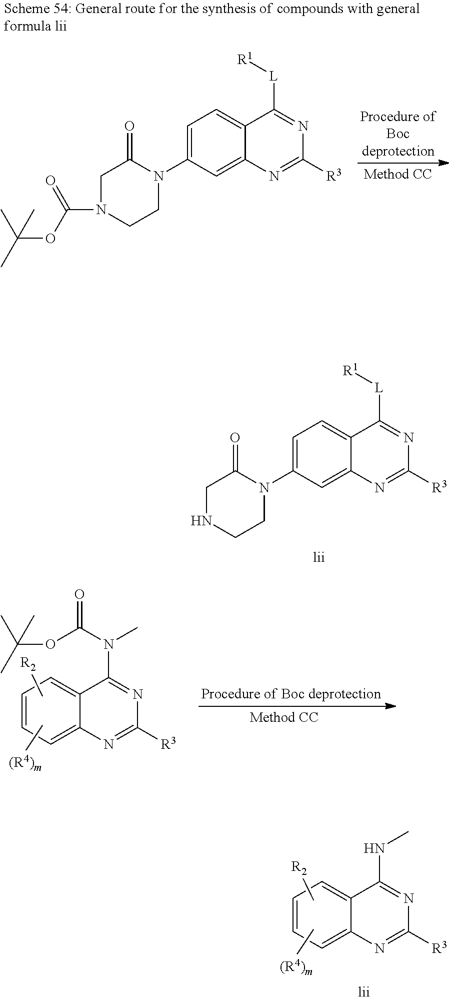

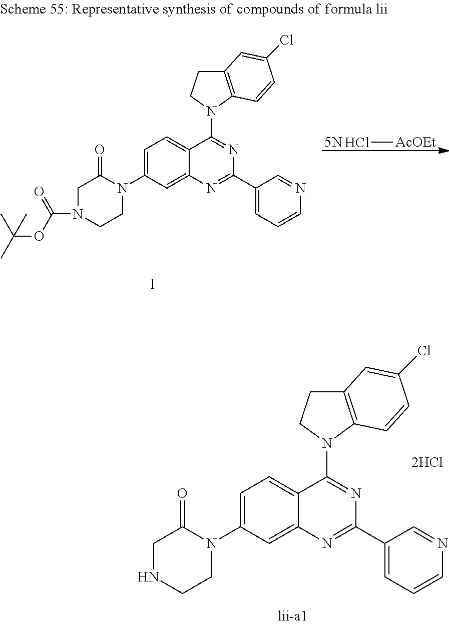

### Method CC

4NHCl-AcOEt (15 ml) was added to Reactant 1 (0.20 g, 0.359 mmol) and the mixture was stirred for 5 hr. To the reaction mixture was added ice-tip and NH3aq. to be basic. Extracted with AcOEt (30 mL*2) and combined organic layers were washed with brine. Dried over Na2SO4 and AcOEt was removed under reduced pressure to give yellow amorphous which was treated with small excess of 5NHCl to give HCl salt of (lii-a1)(0.16 g, 0.30 mmol, 84.11% yield). Structure of the product was confirmed by 1H-NMR ¹H NMR (400 MHz, DMSO) δ 3.33-3.21 (m, 2H), 3.67-3.56 (m, 2H), 3.98-3.94 (m, 2H), 4.18-4.09 (m, 2H), 4.69 (t, J=7.9 Hz, 2H), 7.53-7.18 (m, 2H), 7.72 (dd, J=8.9, 2.2 Hz, 1H), 7.93 (d, J=8.6 Hz, 1H), 8.11-7.99 (m, 2H), 8.35 (d, J=9.0 Hz, 1H), 9.06-8.85 (m, 1H), 9.15 (d, J=7.8 Hz, 1H), 9.64-9.48 (m, 1H), 10.39-10.21 (m, 2H)

TABLE 14

| | | | | | | | | Method | | Reten- | |
|---|---|---|---|---|---|---|---|---|---|---|---|
| | Starting | Starting | | Salt | | ¹H NMR | Purity | of | | tion | LCMS |
| Number | Material 1 | Material 2 | Product | type | ¹H NMR | Solvent | percent | Coupling | LCMS | Time | Method |
| 648 | 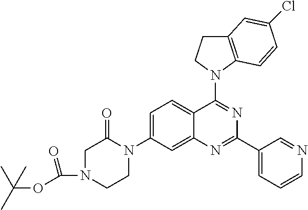 [SMILES: CC(C)(C)OC(=O)N1CCN(c2ccc3c(N4CCc5cc(Cl)ccc54)nc(-c4cccnc4)nc3c2)C(=O)C1] | | 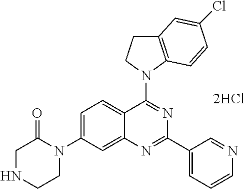 [SMILES: O=C1CNCCN1c1ccc2c(N3CCc4cc(Cl)ccc43)nc(-c3cccnc3)nc2c1] | 2HCl | 1H NMR (400 MHz, DMSO) δ 3.33-3.21 (m, 2H), 3.67-3.56 (m, 2H), 3.98-3.94 (m, 2H), 4.18-4.09 (m, 2H), 4.69 (t, J = 7.9 Hz, 2H), 7.53-7.18 (m, 2H), 7.72 (dd, J = 8.9, 2.2 Hz, 1H), 7.93 (d, J = 8.6 Hz, 1H), 8.11-7.99 (m, 2H), 8.35 (d, J = 9.0 Hz, | DMSO | >98 | CC | | | |
| | | | | | 1H), 9.06-8.85 (m, | | | | | | |
| | | | | | 1H), 9.15 (d, J = | | | | | | |
| | | | | | 7.8 Hz, 1H), 9.64- | | | | | | |
| | | | | | 9.48 (m, 1H), | | | | | | |
| | | | | | 10.39-10.21 (m, | | | | | | |
| | | | | | 2H). | | | | | | |
| 649 | 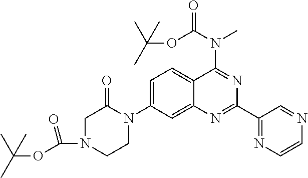 [SMILES: CN(C(=O)OC(C)(C)C)c1nc(-c2cnccn2)nc2cc(N3CCN(C(=O)OC(C)(C)C)CC3=O)ccc12] | | 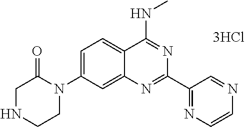 [SMILES: CNc1nc(-c2cnccn2)nc2cc(N3CCNCC3=O)ccc12] | 3HCl | 1H NMR (400 MHz, DMSO) δ 3.61 (s, 2H), 3.98 (s, 2H), 4.12 (t, J = 5.5 Hz, 2H), 7.85 (dd, J = 9.0, 2.1 Hz, 1H), 8.36 (d, J = 2.1 Hz, 1H), 8.77 (d, J = 9.0 Hz, 1H), 9.01 (dd, J = 2.5, 1.5 Hz, | DMSO | >98 | CC | | | |
| | | | | | 1H), 9.07 (d, J = | | | | | | |
| | | | | | 2.5 Hz, 1H), 9.83 | | | | | | |
| | | | | | (d, J = 1.4 Hz, | | | | | | |
| | | | | | 1H), 10.28 (s, | | | | | | |
| | | | | | 2H), 10.95 (d, J = | | | | | | |
| | | | | | 4.8 Hz, 1H), 3.41- | | | | | | |
| | | | | | 3.32 (m, 3H). | | | | | | |
| 650 | 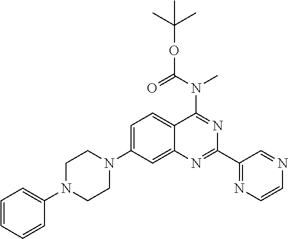 [SMILES: CN(C(=O)OC(C)(C)C)c1nc(-c2cnccn2)nc2cc(N3CCN(c4ccccc4)CC3)ccc12] | | 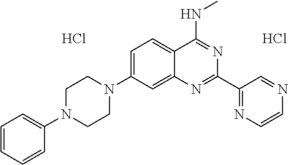 [SMILES: CNc1nc(-c2cnccn2)nc2cc(N3CCN(c4ccccc4)CC3)ccc12 ; Cl ; Cl] | 2HCl | 1H NMR (400 MHz, DMSO) δ 3.44- 3.34 (m, 4H), 3.78-3.61 (m, 4H), 7.18-6.80 (m, 4H), 7.36- 7.20 (m, 3H), 3.32- 3.28 (m, 3H), 7.59-7.48 (m, 2H), 8.44 (d, J = 9.5 Hz, 1H), 8.98 (dd, J = 2.4, 1.5 Hz, 1H), 9.05 (d, J = 2.5 Hz, 1H), 9.77 (d, J = 1.4 Hz, | DMSO | >98 | CC | | | |
| | | | | | 1H), 10.25 (q, | | | | | | |
| | | | | | J = 4.7 Hz, 1H), | | | | | | |
| | | | | | 14.00 (s, 1H). | | | | | | |
| 651 | 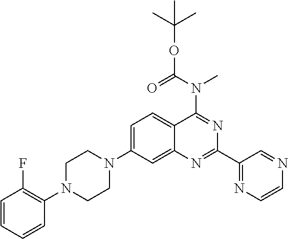 [SMILES: CN(C(=O)OC(C)(C)C)c1nc(-c2cnccn2)nc2cc(N3CCN(c4ccccc4F)CC3)ccc12] | | 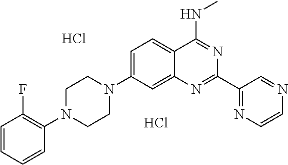 [SMILES: CNc1nc(-c2cnccn2)nc2cc(N3CCN(c4ccccc4F)CC3)ccc12 ; Cl ; Cl] | | 1H NMR (400 MHz, DMSO) δ 3.11 (d, J = 4.4 Hz, 3H), 3.26-3.16 (m, 4H), 3.55 (t, J = 5.0 Hz, 4H), 7.07-6.94 (m, 1H), 7.26-7.07 (m, 3H), 7.40 (dd, J = 9.2, 2.6 Hz, 1H), 8.10 (d, J = 9.2 Hz, 1H), 8.18 (q, J = 4.5 Hz, 1H), 8.72 (d, J = 2.5 Hz, 1H), 8.78 | DMSO | >98 | CC | | | |
| | | | | | (dd, J = 2.5, 1.5 | | | | | | |
| | | | | | Hz, 1H), 9.60 (d, | | | | | | |
| | | | | | J = 1.5 Hz, 1H). | | | | | | |

TABLE 15

| | | | | | | | | Method | | Reten- | |
|---|---|---|---|---|---|---|---|---|---|---|---|
| | Starting | Starting | | Salt | | ¹H NMR | Purity | of | | tion | LCMS |
| Number | Material 1 | Material 2 | Product | type | ¹H NMR | Solvent | percent | Coupling | LCMS | Time | Method |
| 652 | 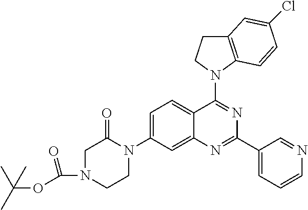 [SMILES: CC(C)(C)OC(=O)N1CCN(c2ccc3c(N4CCc5cc(Cl)ccc54)nc(-c4cccnc4)nc3c2)C(=O)C1] | | 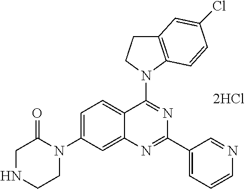 [SMILES: O=C1CNCCN1c1ccc2c(N3CCc4cc(Cl)ccc43)nc(-c3cccnc3)nc2c1] | 2HCl | 1H NMR (400 MHz, DMSO) δ 3.33-3.21 (m, 2H), 3.67-3.56 (m, 2H), 3.98-3.94 (m, 2H), 4.18-4.09 (m, 2H), 4.69 (t, J = 7.9 Hz, 2H), 7.53- 7.18 (m, 2H), 7.72 (dd, J = 8.9, 2.2 Hz, 1H), 7.93 (d, J = 8.6 Hz, 1H), 8.11- 7.99 (m, 2H), 8.35 (d, J = 9.0 Hz, 1H), 9.06-8.85 (m, 1H), | DMSO | >98 | CC | | | |
| | | | | | 9.15 (d, J = 7.8 Hz, | | | | | | |
| | | | | | 1H), 9.64-9.48 (m, | | | | | | |
| | | | | | 1H), 10.39-10.21 | | | | | | |
| | | | | | (m, 2H). | | | | | | |
| 653 | 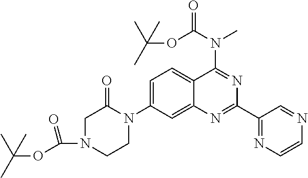 [SMILES: CN(C(=O)OC(C)(C)C)c1nc(-c2cnccn2)nc2cc(N3CCN(C(=O)OC(C)(C)C)CC3=O)ccc12] | | 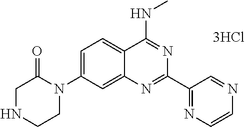 [SMILES: CNc1nc(-c2cnccn2)nc2cc(N3CCNCC3=O)ccc12] | 3HCl | 1H NMR (400 MHz, DMSO) δ 3.61 (s, 2H), 3.98 (s, 2H), 4.12 (t, J = 5.5 Hz, 2H), 7.85 (dd, J = 9.0, 2.1 Hz, 1H), 8.36 (d, J = 2.1 Hz, 1H), 8.77 (d, J = 9.0 Hz, 1H), 9.01 (dd, J = 2.5, 1.5 Hz, | DMSO | >98 | CC | | | |
| | | | | | 1H), 9.07 (d, J = | | | | | | |
| | | | | | 2.5 Hz, 1H), 9.83 | | | | | | |
| | | | | | (d, J = 1.4 Hz, | | | | | | |
| | | | | | 1H), 10.28 (s, 2H), | | | | | | |
| | | | | | 10.95 (d, J = | | | | | | |
| | | | | | 4.8 Hz, 1H), 3.41- | | | | | | |
| | | | | | 3.32 (m, 3H). | | | | | | |
| 654 | 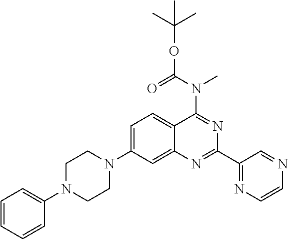 [SMILES: CN(C(=O)OC(C)(C)C)c1nc(-c2cnccn2)nc2cc(N3CCN(c4ccccc4)CC3)ccc12] | | 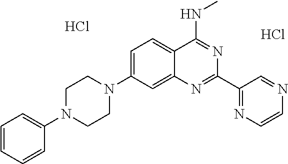 [SMILES: CNc1nc(-c2cnccn2)nc2cc(N3CCN(c4ccccc4)CC3)ccc12 ; Cl ; Cl] | 2HCl | 1H NMR (400 MHz, DMSO) δ 3.44-3.34 (m, 4H) 3.78-3.61 (m, 4H), 7.18-6.80 (m, 4H), 7.36- 7.20 (m, 3H), 3.32- 3.28 (m, 3H), 7.59-7.48 (m, 2H), 8.44 (d, J = 9.5 Hz, 1H), 8.98 (dd, J = 2.4, 1.5 Hz, 1H), 9.05 (d, J = 2.5 Hz, 1H), 9.77 (d, J = 1.4 Hz, | DMSO | >98 | CC | | | |
| | | | | | 1H), 10.25 (q, | | | | | | |
| | | | | | J = 4.7 Hz, 1H), | | | | | | |
| | | | | | 14.00 (s, 1H). | | | | | | |
| 655 | 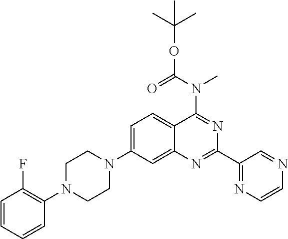 [SMILES: CN(C(=O)OC(C)(C)C)c1nc(-c2cnccn2)nc2cc(N3CCN(c4ccccc4F)CC3)ccc12] | | 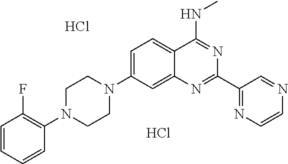 [SMILES: CNc1nc(-c2cnccn2)nc2cc(N3CCN(c4ccccc4F)CC3)ccc12 ; Cl ; Cl] | | 1H NMR (400 MHz, DMSO) δ 3.11 (d, J = 4.4 Hz, 3H), 3.26-3.16 (m, 4H), 3.55 (t, J = 5.0 Hz, 4H), 7.07-6.94 (m, 1H), 7.26-7.07 (m, 3H), 7.40 (dd, J = 9.2, 2.6 Hz, 1H), 8.10 (d, J = 9.2 Hz, 1H), 8.18 (q, J = 4.5 Hz, 1H), 8.72 (d, J = 2.5 Hz, 1H), 8.78 | DMSO | >98 | CC | | | |
| | | | | | (dd, J = 2.5, 1.5 | | | | | | |
| | | | | | Hz, 1H), 9.60 (d, | | | | | | |
| | | | | | J = 1.5 Hz, 1H). | | | | | | |

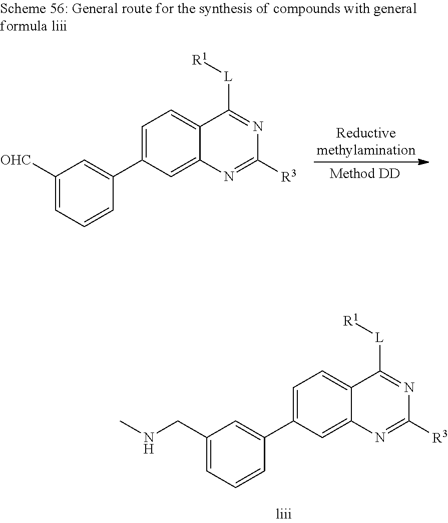

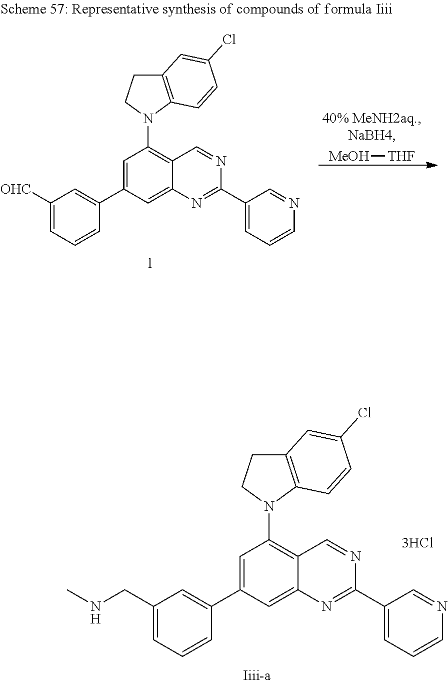

### Method DD

A solution of Reactant 1 (0.24 g, 0.518 mmol) and 40% Methylamine (0.201 g, 2.59 mmol) in MeOH-THE (10-10 ml) was stirred for 2 hr (dissolved). To a stirring solution was added Sodium borohydride (0.039 g, 1.037 mmol) and the mixture was stirred over night. The reaction mixture was quenched by small amount of H2O and evaporated. Extraction with CH2Cl2 (20 mL*2) and then combined organic layers were washed with H2O and brine. Dried over Na2SO4 and CH2Cl2 was removed under reduced pressure to give crude solid which was washed with ether to give a pale yellow solid. The solid was treated with small excess of 5NHClaq (0.5 ml) to give hydrochloride salt. The obtained hydrochloride salt was washed with Ether-ethanol to afford (liii-a) (0.17 g, 0.29 mmol, 55.83% yield) as a yellow solid. Structure of the product was confirmed by 1H-NMR. ¹H NMR (400 MHz, DMSO) δ 2.60 (t, J=5.3 Hz, 3H), 3.28 (t, J=7.8 Hz, 2H), 4.25 (t, J=5.8 Hz, 2H), 4.73 (t, J=7.9 Hz, 2H), 7.36 (dd, J=8.6, 2.3 Hz, 1H), 7.49 (d, J=2.2 Hz, 1H), 7.72-7.60 (m, 2H), 8.12-7.87 (m, 3H), 8.20 (s, 1H), 8.49-8.33 (m, 1H), 8.97 (dd, J=5.3, 1.6 Hz, 1H), 9.13 (d, J=8.3 Hz, 1H), 9.40 (s, 2H), 9.60 (d, J=2.0 Hz, 1H).

TABLE 16

| | | | | | | | | Method | | Reten- | |
|---|---|---|---|---|---|---|---|---|---|---|---|
| | Starting | Starting | | Salt | | ¹H NMR | Purity | of | | tion | LCMS |
| Number | Material 1 | Material 2 | Product | type | ¹H NMR | Solvent | percent | Coupling | LCMS | Time | Method |
| 656 | 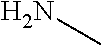 | 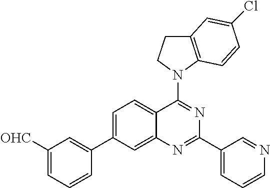 [SMILES: O=Cc1cccc(-c2ccc3c(N4CCc5cc(Cl)ccc54)nc(-c4cccnc4)nc3c2)c1] | 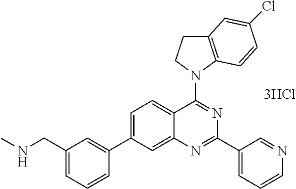 [SMILES: CNCc1cccc(-c2ccc3c(N4CCc5cc(Cl)ccc54)nc(-c4cccnc4)nc3c2)c1] | 3HCl | 1H NMR (100 MHz, DMSO) δ 2.60 (t, J = 5.3 Hz, 3H), 3.28 (t, J = 7.8 Hz, 2H), 4.25 (t, J = 5.8 Hz, 2H), 4.73 (t, J = 7.9 Hz, 2H), 7.36 (dd, J = 8.6, 2.3 Hz, 1H), 7.49 (d, J = 2.2 Hz, 1H), 7.72- 7.60 (m, 2H), 8.12-7.87 (m, 3H), | DMSO | >98 | DD | | | |
| | | | | | 8.20 (s, 1H), | | | | | | |
| | | | | | 8.49-8.33 (m, 1H), | | | | | | |
| | | | | | 8.97 (dd, J = | | | | | | |
| | | | | | 5.3, 1.6 Hz, 1H), | | | | | | |
| | | | | | 9.13 (d, J = 8.3 | | | | | | |
| | | | | | Hz, 1H), 9.40 (s, | | | | | | |
| | | | | | 2H), 9.60 (d, J = | | | | | | |
| | | | | | 2.0 Hz, 1H). | | | | | | |
| 657 | 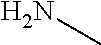 | 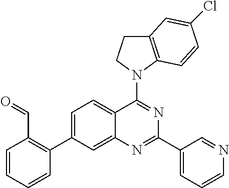 [SMILES: O=Cc1ccccc1-c1ccc2c(N3CCc4cc(Cl)ccc43)nc(-c3cccnc3)nc2c1] | 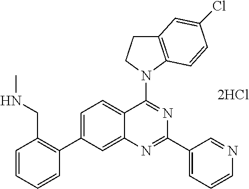 [SMILES: CNCc1ccccc1-c1ccc2c(N3CCc4cc(Cl)ccc43)nc(-c3cccnc3)nc2c1] | 3HCl | 1H NMR (400 MHz, DMSO) δ 2.48- 2.42 (m, 3H), 4.19-4.10 (m, 2H), 4.74 (t, J = 7.9 Hz, 2H), 7.37 (dd, J = 8.6, 2.3 Hz, 1H), 3.35- 3.24 (m, 2H), 7.49 (q, J = 3.1, 2.4 Hz, 2H), 7.65-7.53 (m, 2H), 7.70 (dd, J = 8.6, 1.9 Hz, | DMSO | >98 | DD | | | |
| | | | | | 1H), 8.00- | | | | | | |
| | | | | | 7.89 (m, 3H), | | | | | | |
| | | | | | 8.03 (d, J = 1.9 Hz, | | | | | | |
| | | | | | 1H), 8.38 (d, J = | | | | | | |
| | | | | | 8.7 Hz, 1H), 8.93 | | | | | | |
| | | | | | (dd, J = 5.2, 1.6 | | | | | | |
| | | | | | Hz, 1H), 9.07 (dt, | | | | | | |
| | | | | | J - 8.1, 1.8 Hz, | | | | | | |
| | | | | | 1H), 9.65-9.43 | | | | | | |
| | | | | | (m, 3H). | | | | | | |
| 658 | 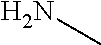 | 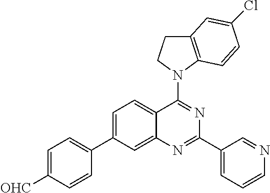 [SMILES: O=Cc1ccc(-c2ccc3c(N4CCc5cc(Cl)ccc54)nc(-c4cccnc4)nc3c2)cc1] | 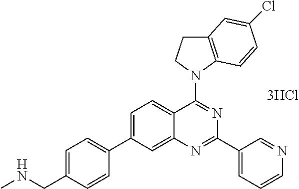 [SMILES: CNCc1ccc(-c2ccc3c(N4CCc5cc(Cl)ccc54)nc(-c4cccnc4)nc3c2)cc1] | 3HCl | 1H NMR (400 MHz, DMSO) δ 2.61- 2.55 (m, 3H), 3.24 (s, 0H), 4.21 (t, J = 5.7 Hz, 2H), 4.69 (t, J = 7.9 Hz, 2H), 7.35 (dd, J = 8.7, 2.3 Hz, 1H), 7.47 (d, J = 2.2 Hz, 1H), 7.84- 7.69 (m, 2H), 8.06-7.85 (m, 5H), 8.41-8.28 | DMSO | >98 | DD | | | |
| | | | | | (m, 2H), 8.94 | | | | | | |
| | | | | | (dd, J = 5.3, 1.6 | | | | | | |
| | | | | | Hz, 1H), 9.08 (dt, | | | | | | |
| | | | | | J = 8.1, 1.8 Hz, | | | | | | |
| | | | | | 1H), 9.67-9.43 | | | | | | |
| | | | | | (m, 3H). | | | | | | |

TABLE 17

| Number | Starting Material 1 | Starting Material 2 | Product | Salt type |
|---|---|---|---|---|
| 659 | 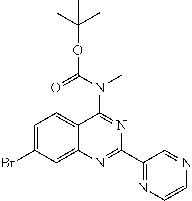 [SMILES: CN(C(=O)OC(C)(C)C)c1nc(-c2cnccn2)nc2cc(Br)ccc12] | 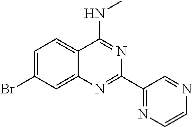 [SMILES: CNc1nc(-c2cnccn2)nc2cc(Br)ccc12] | 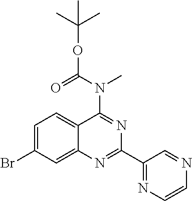 [SMILES: CN(C(=O)OC(C)(C)C)c1nc(-c2cnccn2)nc2cc(Br)ccc12] | |

| | | ¹H NMR | | | | | |
|---|---|---|---|---|---|---|---|
| Number | ¹H NMR | Solvent | Purity percent | Method of Coupling | LCMS | Retention Time | LCMS Method |
| 659 | 1H NMR (400 MHz, CDCl3) δ 1.39 (s, 10H), 7.76-7.69 (m, 1H), 7.87- 7.80 (m, 1H), 8.46 (dd, J = 1.9, 0.5 Hz, 1H), 8.73 (d, J = 2.4 Hz, 1H), 8.85 (dd, J = 2.5, 1.5 Hz, 1H), 9.87-9.80 (m, 1H). | CDCl3 | >98 | Similar with xxxviii-a in Scheme46 | | | |

TABLE 18

| | Starting | Starting | | Salt | |
|---|---|---|---|---|---|
| Number | Material 1 | Material 2 | Product | type | ¹H NMR |
| 660 | 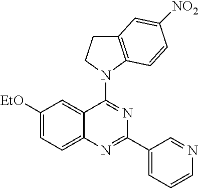 [SMILES: CCOc1ccc2nc(-c3cccnc3)nc(N3CCc4cc([N+](=O)[O-])ccc43)c2c1] | | 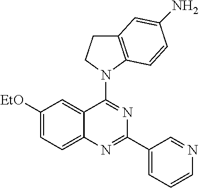 [SMILES: CCOc1ccc2nc(-c3cccnc3)nc(N3CCc4cc(N)ccc43)c2c1] | | 1H NMR (400 MHz, CDCl3) δ 1.45 (t, J = 7.0 Hz, 3H), 3.17 (t, J = 7.8 Hz, 2H), 4.03 (q, J = 7.0 Hz, 2H), 4.47 (t, J = 7.9 Hz, 2H), 6.51 (dd, J = 8.5, 2.4 Hz, 1H), 6.70 (d, J = 2.4 Hz, 1H), 7.13 (d, J = 8.5 Hz, 1H), 7.30 (d, J = 2.8 Hz, 1H), 7.52- 7.34 (m, 2H), 7.92 (d, J = 9.2 Hz, 1H), 8.66 (dd, J = 4.9, 1.7 Hz, 1H), 8.76 (dt, J = 8.0, 2.0 Hz, 1H), 9.71 (dd, J = 2.1, 0.9 Hz, 1H). |

| | ¹H NMR | | Method | | | |
|---|---|---|---|---|---|---|
| | Solvent | Purity | of | | Retention | LCMS |
| Number | | percent | Coupling | LCMS | Time | Method |
| 660 | CDCl3 | >98 | Similar with | | | |
| | | | xxvi-a in | | | |
| | | | Scheme 41 | | | |

TABLE 19

| | | | | | | | | Method | | | |
|---|---|---|---|---|---|---|---|---|---|---|---|
| | | | | | | ¹H | | of | | Reten- | |
| Num- | Starting | Starting | | Salt | | NMR | Purity | Cou- | | tion | LCMS |
| ber | Material 1 | Material 2 | Product | type | ¹H NMR | Solvent | percent | pling | LCMS | Time | Method |
| 661 | 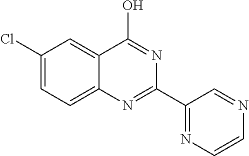 [SMILES: Oc1nc(-c2cnccn2)nc2ccc(Cl)cc12] | | 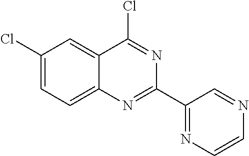 [SMILES: Clc1ccc2nc(-c3cnccn3)nc(Cl)c2c1] | | 1H NMR (400 MHz, CDCl3) δ 7.96 (dd, J = 9.0, 2.4 Hz, 1H), 8.24 (dd, J = 8.9, 0.5 Hz, 1H), 8.33 (dd, J = 2.2, 0.6 Hz, 1H), 8.75 (d, J = 2.4 Hz, 1H), 8.86 (dd, J = 2.4, 1.5 Hz, 1H), 9.86 (d, J = 1.5 Hz, 1H). | CDCl3 | >98 | Method D F | | | |
| 662 | 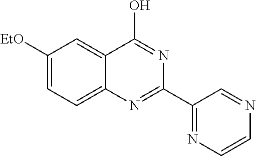 [SMILES: CCOc1ccc2nc(-c3cnccn3)nc(O)c2c1] | | 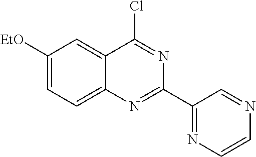 [SMILES: CCOc1ccc2nc(-c3cnccn3)nc(Cl)c2c1] | | 1H NMR (400 MHz, CDCl3) δ 1.58- 1.49 (m, 3H), 4.26 (q, J = 7.0 Hz, 2H), 7.50 (d, J = 2.7 Hz, 1H), 7.64 (dd, J = 9.2, 2.8 Hz, 1H), 8.18 (d, J = 9.2 Hz, 1H), 8.70 (d, J = 2.4 Hz, 1H), 8.83 (dd, J = 2.4, 1.5 Hz, 1H), 9.83 (d, J = 1.5 Hz, 1H). | CDCl3 | >98 | Method D F | | | |
| 663 | 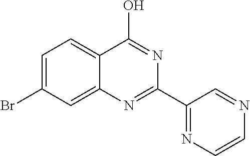 [SMILES: Oc1nc(-c2cnccn2)nc2cc(Br)ccc12] | | 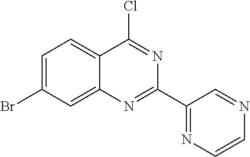 [SMILES: Clc1nc(-c2cnccn2)nc2cc(Br)ccc12] | | 1H NMR (400 MHz, CDCl3) δ 7.87 (dd, J = 8.8, 1.9 Hz, 1H), 8.20 (d, J = 8.9 Hz, 1H), 8.48 (d, J = 1.8 Hz, 1H), 8.75 (d, J = 2.4 Hz, 1H), 8.86 (dd, J = 2.5, 1.5 Hz, 1H), 9.87-9.82 (m, 1H). | CDCl3 | >98 | Method D F | | | |
| 664 | 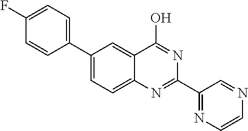 [SMILES: Oc1nc(-c2cnccn2)nc2ccc(-c3ccc(F)cc3)cc12] | | 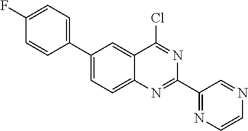 [SMILES: Fc1ccc(-c2ccc3nc(-c4cnccn4)nc(Cl)c3c2)cc1] | | 1H NMR (400 MHz, CDCl3) δ 7.29- 7.19 (m, 2H), 7.78-7.68 (m, 2H), 8.27-8.20 (m, 1H), 8.38- 8.32 (m, 1H), 8.43 (d, J = 2.0 Hz, 1H), 8.74 (d, J = 2.4 Hz, 1H), 8.86 (dd, J = 2.4, 1.5 Hz, 1H), 9.88 (d, J = 1.5 Hz, 1H). | CDCl3 | >98 | Method D, F | | | |

TABLE 20

| | Starting | Starting | | Salt | | ¹H NMR | Purity | Method | | Retention | LCMS |
|---|---|---|---|---|---|---|---|---|---|---|---|
| Number | Material 1 | Material 2 | Product | type | ¹H NMR | Solvent | percent | of Coupling | LCMS | Time | Method |
| 665 | 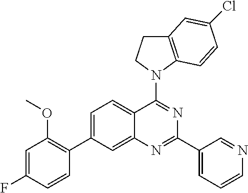 [SMILES: COc1cc(F)ccc1-c1ccc2c(N3CCc4cc(Cl)ccc43)nc(-c3cccnc3)nc2c1] | | 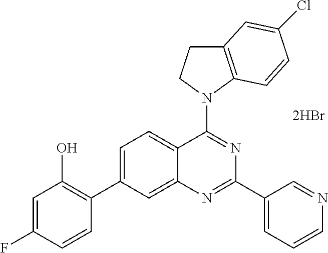 [SMILES: Oc1cc(F)ccc1-c1ccc2c(N3CCc4cc(Cl)ccc43)nc(-c3cccnc3)nc2c1] | 2HBr | 1H NMR (400 MHz, DMSO) δ 3.87 (s, 1H), 4.71 (t, J = 7.9 Hz, 5H), 6.92-6.77 (m, 2H), 7.32 (ddd, J = 26.0, 8.6, 2.3 Hz, 1H), 3.35- 3.17 (m, 6H), 7.58-7.43 (m, 2H), 8.02-7.81 (m, 3H), 8.11 (dd, J = 10.0, 1.8 Hz, 1H), 8.34-8.21 (m, 1H), 9.07-8.85 (m, 1H), 9.70- | DMSO | <98 | Method A | | | |
| | | | | | 9.38 (m, 1H), | | | | | | |
| | | | | | 10.59-10.36 (m, | | | | | | |
| | | | | | 1H). | | | | | | |
| 666 | 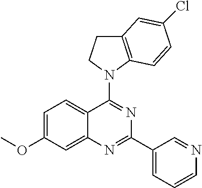 [SMILES: COc1ccc2c(N3CCc4cc(Cl)ccc43)nc(-c3cccnc3)nc2c1] | | 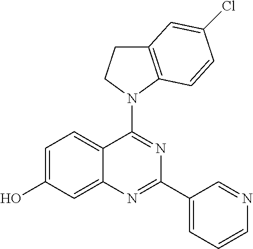 [SMILES: Oc1ccc2c(N3CCc4cc(Cl)ccc43)nc(-c3cccnc3)nc2c1] | | 1H NMR (400 MHz, DMSO) δ 3.22 (t, J = 8.0 Hz, 2H), 4.54 (t, J = 8.0 Hz, 2H), 7.10 (dd, J = 9.1, 2.6 Hz, 1H), 7.19 (d, J = 2.5 Hz, 1H), 7.28 (dd, J = 8.5, 2.4 Hz, 1H), 7.47- 7.36 (m, 1H), 7.54 (dd, J = 7.7, 4.8 Hz, 1H), 7.63 (d, J = 8.6 Hz, 1H), 8.14-7.94 (m, 1H), 8.78-8.60 | DMSO | >98 | Method A | | | |
| | | | | | (m, 2H), 9.53 (s, | | | | | | |
| | | | | | 1H), 10.71 (s, | | | | | | |
| | | | | | 1H). | | | | | | |

TABLE 21

| | | | | | | | Puri- | Method | | | |
|---|---|---|---|---|---|---|---|---|---|---|---|
| | | | | | | ¹H | ty | of | | Reten- | |
| Num- | Starting | Starting | | Salt | | NMR | per- | Cou- | | tion | LCMS |
| ber | Material 1 | Material 2 | Product | type | ¹H NMR | Solvent | cent | pling | LCMS | Time | Method |
| 667 | 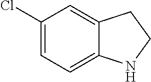 | 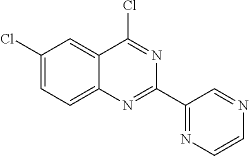 [SMILES: Clc1ccc2nc(-c3cnccn3)nc(Cl)c2c1] | 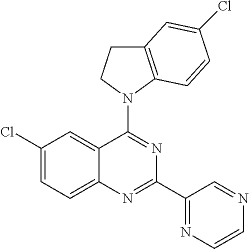 [SMILES: Clc1ccc2c(c1)CCN2c1nc(-c2cnccn2)nc2ccc(Cl)cc12] | | 1H NMR (400 MHz, DMSO) δ 4.71 (t, J = 7.9 Hz, 2H), 7.56-7.30 (m, 4H), 7.67 (ddd, J = 9.2, 6.1, 3.2 Hz, 1H), 7.85 (dt, J = 8.7, 1.8 Hz, 1H), 8.02-7.91 (m, 2H), 8.21 (t, J = 1.6 Hz, 1H), 8.38 (d, J = 8.8 Hz, 1H), 8.93 (dd, J = 5.3, 1.6 Hz, 1H), 9.09 (dt, J = 8.2, 1.8 Hz, 1H), | DMSO | >98 | Method D F3, G2 | | | |
| | | | | | 9.58 (dd, J = | | | | | | |
| | | | | | 2.2, 0.7 Hz, 1H), | | | | | | |
| | | | | | 3.31-3.18 (m, 2H).. | | | | | | |
| 668 | 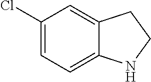 | 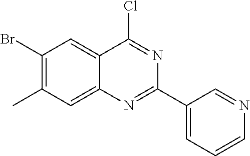 [SMILES: Cc1cc2nc(-c3cccnc3)nc(Cl)c2cc1Br] | 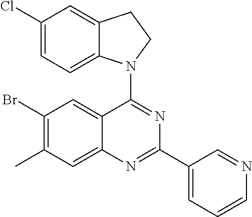 [SMILES: Cc1cc2nc(-c3cccnc3)nc(N3CCc4cc(Cl)ccc43)c2cc1Br] | | 1H NMR (400 MHz, DMSO) δ 2.57 (s, 3H), 3.24 (t, J = 7.9 Hz, 2H), 4.63 (t, J = 8.0 Hz, 2H), 7.38- 7.29 (m, 1H), 7.44 (s, 1H), 7.57 (dd, J = 8.0, 4.7 Hz, 1H), 7.80 (d, J = 8.7 Hz, 1H), 7.96 (s, 1H), 8.38 (s, 1H), 8.69 (dd, J = 14.1, 6.3 Hz, | DMSO | >98 | Method D F3, G2 | | | |
| | | | | | 2H), 9.53 (s, 1H). | | | | | | |
| 669 | 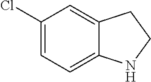 | 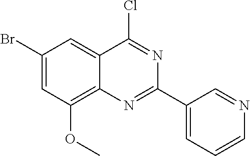 [SMILES: COc1cc(Br)cc2c(Cl)nc(-c3cccnc3)nc12] | 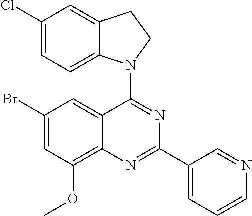 [SMILES: COc1cc(Br)cc2c(N3CCc4cc(Cl)ccc43)nc(-c3cccnc3)nc12] | | 1H NMR (400 MHz, CDCl3) δ 3.25 (t, J = 7.9 Hz, 2H), 4.11 (s, 3H), 4.52 (t, J = 7.9 Hz, 2H), 7.16 (dd, J = 8.6, 2.2 Hz, 1H), 7.34-7.23 (m, 3H), 7.40 (ddd, J = 8.0, 4.8, 0.9 Hz, 1H), 7.79- 7.69 (m, 1H), 8.73-8.63 (m, 1H), 8.78 (dt, J = | CDCl3 | >98 | Method D F3, G2 | | | |
| | | | | | 7.9, 2.0 Hz, 1H), | | | | | | |
| | | | | | 9.73-9.65 (m, | | | | | | |
| | | | | | 1H). | | | | | | |
| 670 | 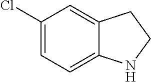 | 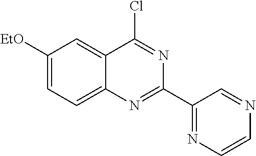 [SMILES: CCOc1ccc2nc(-c3cnccn3)nc(Cl)c2c1] | 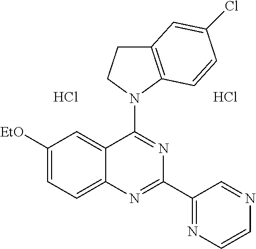 [SMILES: CCOc1ccc2nc(-c3cnccn3)nc(N3CCc4cc(Cl)ccc43)c2c1 ; Cl ; Cl] | 2HCl | 1H NMR (400 MHz, DMSO) δ 1.42 (t, J = 6.9 Hz, 3H), 4.23 (q, J = 6.9 Hz, 2H), 4.84 (t, J = 7.6 Hz, 2H), 7.48 (dd, J = 8.9, 2.3 Hz, 1H), 3.34-3.24 (m, 2H), 7.55 (d, J = 2.2 Hz, 1H), 7.65 (d, J = 2.6 Hz, 1H), 7.79 (dd, J = 9.3, 2.6 Hz, 1H), 8.07 (d, J = 8.6 Hz, 1H), 8.23 | DMSO | >98 | Method D F3, G2 | | | |
| | | | | | (d, J = 9.2 Hz, | | | | | | |
| | | | | | 1H), 9.01-8.92 | | | | | | |
| | | | | | (m, 2H), 9.54 | | | | | | |
| | | | | | (d, J = 1.4 Hz, 1H). | | | | | | |
| 671 | 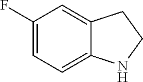 | 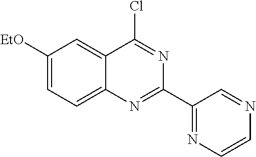 [SMILES: CCOc1ccc2nc(-c3cnccn3)nc(Cl)c2c1] | 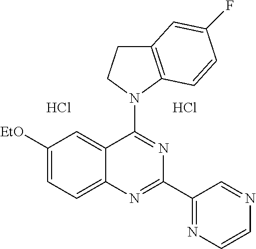 [SMILES: CCOc1ccc2nc(-c3cnccn3)nc(N3CCc4cc(F)ccc43)c2c1 ; Cl ; Cl] | 2HCl | 1H NMR (400 MHz, DMSO) δ 1.42 (t, J = 6.9 Hz, 3H), 3.32-3.24 (m, 2H), 4.24 (q, J = 6.9 Hz, 2H), 4.86 (t, J = 7.7 Hz, 2H), 7.39- 7.22 (m, 2H), 7.67 (d, J = 2.5 Hz, 1H), 7.78 (dd, J = 9.2, 2.6 Hz, 1H), 8.27-8.08 (m, 2H), 9.01- 8.91 (m, 2H), 9.54 (d, J = 1.4 Hz, | DMSO | >98 | Method D F3, G2 | | | |
| | | | | | 1H). | | | | | | |
| 672 | 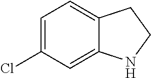 | 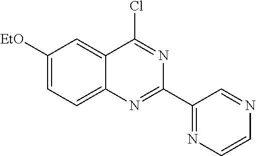 [SMILES: CCOc1ccc2nc(-c3cnccn3)nc(Cl)c2c1] | 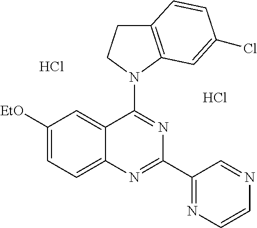 [SMILES: CCOc1ccc2nc(-c3cnccn3)nc(N3CCc4ccc(Cl)cc43)c2c1 ; Cl ; Cl] | 2HCl | 1H NMR (400 MHz, CDCl3) δ 1.53- 1.41 (m, 3H), 3.24 (t, J = 8.0 Hz, 2H), 4.05 (q, J = 7.0 Hz, 2H), 4.56 (t, J = 8.0 Hz, 2H), 6.94 (dd, J = 7.9, 1.9 Hz, 1H), 7.08 (d, J = 1.8 Hz, 1H), 7.24- 7.17 (m, 2H), 7.55 (dd, J = 9.2, 2.7 Hz, 1H), 8.19 (d, J = 9.2 Hz, | CDCl3 | >98 | Method D F3, G2 | | | |
| | | | | | 1H), 8.66 (d, J = | | | | | | |
| | | | | | 2.4 Hz, 1H), | | | | | | |
| | | | | | 8.81 (dd, J = 2.5, | | | | | | |
| | | | | | 1.5 Hz, 1H), 9.76 | | | | | | |
| | | | | | (d, J = 1.5 Hz, | | | | | | |
| | | | | | 1H). | | | | | | |
| 673 | 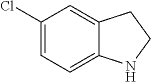 | 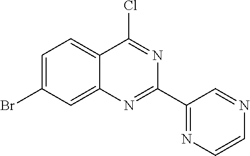 [SMILES: Clc1nc(-c2cnccn2)nc2cc(Br)ccc12] | 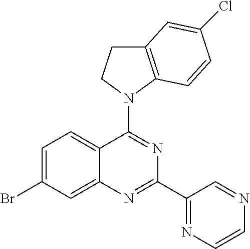 [SMILES: Clc1ccc2c(c1)CCN2c1nc(-c2cnccn2)nc2cc(Br)ccc12] | | 1H NMR (400 MHz, CDCl3) δ 3.27 (t, J = 7.9 Hz, 2H), 4.57 (t, J = 7.9 Hz, 2H), 7.17 (dd, J = 8.6, 2.2 Hz, 1H), 7.33-7.27 (m, 1H), 7.42 (d, J = 8.7 Hz, 1H), 7.60 (dd, J = 8.9, 2.0 Hz, 1H), 7.94 (d, J = 8.9 Hz, 1H), 8.41 (d, J = 2.0 Hz, 1H), 8.69 (d, J = 2.4 Hz, 1H), 8.81 (dd, J = | CDCl3 | >98 | Method D F3, G2 | | | |
| | | | | | 2.5, 1.5 Hz, 1H), | | | | | | |
| | | | | | 9.72 (d, J = 1.5 | | | | | | |
| | | | | | Hz, 1H). | | | | | | |
| 674 | 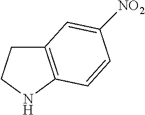 [SMILES: O=[N+]([O-])c1ccc2c(c1)CCN2] | 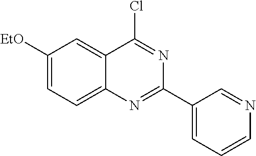 [SMILES: CCOc1ccc2nc(-c3cccnc3)nc(Cl)c2c1] | 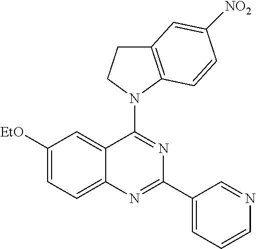 [SMILES: CCOc1ccc2nc(-c3cccnc3)nc(N3CCc4cc([N+](=O)[O-])ccc43)c2c1] | | 1H NMR (400 MHz, DMSO) δ 1.39 (t, J = 6.9 Hz, 3H), 3.39-3.34 (m, 3H), 4.13 (q, J = 7.0 Hz, 2H), 4.67 (t, J = 8.3 Hz, 2H), 7.35 (d, J = 2.7 Hz, 1H), 7.45 (d, J = 8.9 Hz, 1H), 7.63-7.50 (m, 1H), 7.67 (dd, J = 9.2, 2.6 Hz, 1H), 8.01 (d, J = 9.1 Hz, 1H), 8.30- 8.09 (m, 2H), | DMSO | >98 | Method D F3, G2 | | | |
| | | | | | 8.80-8.58 (m, | | | | | | |
| | | | | | 2H), 9.57 (dd, J = | | | | | | |
| | | | | | 2.1, 0.9 Hz, 1H) | | | | | | |
| 675 | 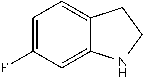 | 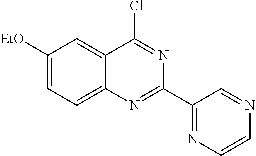 [SMILES: CCOc1ccc2nc(-c3cnccn3)nc(Cl)c2c1] | 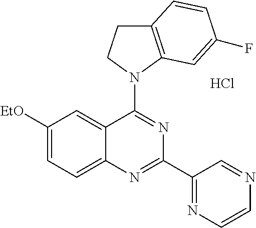 [SMILES: CCOc1ccc2nc(-c3cnccn3)nc(N3CCc4ccc(F)cc43)c2c1 ; Cl] | HCl | 1H NMR (400 MHz, DMSO) δ 1.45- 1.36 (m, 3H), 3.25 (t, J = 7.7 Hz, 2H), 4.24 (q, J = 6.9 Hz, 2H), 4.85 (t, J = 7.7 Hz, 2H), 7.03 (td, J = 8.7, 2.5 Hz, 1H), 7.52-7.40 (m, 1H), 7.66 (d, J = 2.8 Hz, 1H), 7.78 (dt, J = 9.2, 2.0 Hz, 1H), 7.98- 7.85 (m, 1H), 8.19 | DMSO | >98 | Method D F3, G2 | | | |
| | | | | | (d, J = 9.3 Hz, | | | | | | |
| | | | | | 1H), 9.03-8.90 | | | | | | |
| | | | | | (m, 2H), 9.58- | | | | | | |
| | | | | | 9.50 (m, 1H). | | | | | | |
| 676 | 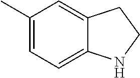 | 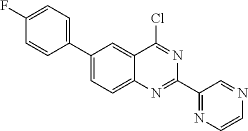 [SMILES: Fc1ccc(-c2ccc3nc(-c4cnccn4)nc(Cl)c3c2)cc1] | 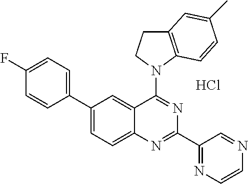 [SMILES: Cc1ccc2c(c1)CCN2c1nc(-c2cnccn2)nc2ccc(-c3ccc(F)cc3)cc12 ; Cl] | HCl | 1H NMR (400 MHz, DMSO) δ 2.39 (s, 3H), 3.25 (s, 2H), 4.96 (t, J = 7.4 Hz, 2H), 7.35- 7.21 (m, 2H), 7.47-7.35 (m, 2H), 7.99-7.86 (m, 2H), 8.21 (d, J = 8.0 Hz, 1H), 8.37-8.26 (m, 1H), 8.48-8.37 | DMSO | >98 | Method D R4, F3, G2 | | | |
| | | | | | (m, 1H), 8.58 (d, | | | | | | |
| | | | | | J = 2.0 Hz, 1H), | | | | | | |
| | | | | | 9.09-8.96 (m, | | | | | | |
| | | | | | 2H), 9.57 (d, J = | | | | | | |
| | | | | | 1.4 Hz, 1H). | | | | | | |
| 677 | 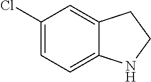 | 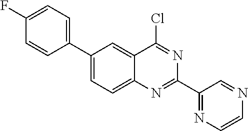 [SMILES: Fc1ccc(-c2ccc3nc(-c4cnccn4)nc(Cl)c3c2)cc1] | 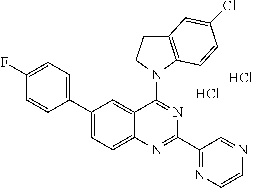 [SMILES: Fc1ccc(-c2ccc3nc(-c4cnccn4)nc(N4CCc5cc(Cl)ccc54)c3c2)cc1 ; Cl ; Cl] | 2HCl | 1H NMR (400 MHz, DMSO) δ 3.36- 3.27 (m, 2H), 4.93 (t, J = 7.6 Hz, 2H), 7.44- 7.36 (m, 2H), 7.60- 7.45 (m, 2H), 7.97-7.88 (m, 2H), 8.31-8.16 (m, 2H), 8.41- 8.33 (m, 1H), 8.53 (d, J = 2.0 Hz, | DMSO | >98 | Method D, R4, F3, G2 | | | |
| | | | | | 1H), 9.03-8.92 | | | | | | |
| | | | | | (m, 2H), 9.61- | | | | | | |
| | | | | | 9.55 (m, 1H). | | | | | | |
| 678 | 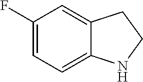 | 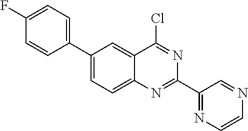 [SMILES: Fc1ccc(-c2ccc3nc(-c4cnccn4)nc(Cl)c3c2)cc1] | 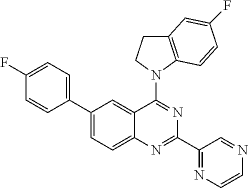 [SMILES: Fc1ccc(-c2ccc3nc(-c4cnccn4)nc(N4CCc5cc(F)ccc54)c3c2)cc1] | | 1H NMR (400 MHz, CDCl3) δ 3.29 (t, J = 7.9 Hz, 2H), 4.64 (t, J = 8.0 Hz, 2H), 6.92 (td, J = 8.8, 2.7 Hz, 1H), 7.11-6.99 (m, 1H), 7.21- 7.11 (m, 2H), 7.48 (dd, J = 8.8, 4.5 Hz, 1H), 7.67- 7.55 (m, 2H), | CDCl3 | >98 | Method D, R4, F3, G2 | | | |
| | | | | | 8.12-8.02 (m, | | | | | | |
| | | | | | 1H), 8.26-8.17 | | | | | | |
| | | | | | (m, 1H), 8.29 (d, | | | | | | |
| | | | | | J = 8.7 Hz, 1H), | | | | | | |
| | | | | | 8.68 (d, J = 2.4 | | | | | | |
| | | | | | Hz, 1H), 8.83 (dd, | | | | | | |
| | | | | | J = 2.5, 1.5 Hz, | | | | | | |
| | | | | | 1H), 9.76 (d, J = | | | | | | |
| | | | | | 1.4 Hz, 1H). | | | | | | |
| 679 | 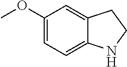 | 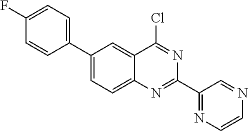 [SMILES: Fc1ccc(-c2ccc3nc(-c4cnccn4)nc(Cl)c3c2)cc1] | 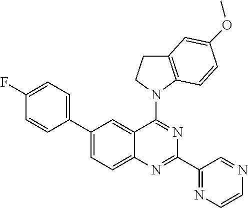 [SMILES: COc1ccc2c(c1)CCN2c1nc(-c2cnccn2)nc2ccc(-c3ccc(F)cc3)cc12] | | 1H NMR (400 MHz, CDCl3) δ 3.27 (t, J = 7.8 Hz, 2H), 3.85 (s, 3H), 4.62 (t, J = 7.9 Hz, 2H), 6.78 (dd, J = 8.8, 2.7 Hz, 1H), 6.99-6.86 (m, 1H), 7.23- 7.09 (m, 2H), 7.68- 7.52 (m, 3H), 8.03 (dd, J = 8.7, 2.0 Hz, 1H), 8.31- | CDCl3 | >98 | Method D, R4, F3, G2 | | | |
| | | | | | 8.21 (m, 2H), | | | | | | |
| | | | | | 8.67 (d, J = 2.5 Hz, | | | | | | |
| | | | | | 1H), 8.82 (dd, | | | | | | |
| | | | | | J = 2.4, 1.5 Hz, | | | | | | |
| | | | | | 1H), 9.77 (d, J = | | | | | | |
| | | | | | 1.4 Hz, 1H). | | | | | | |
| 680 | 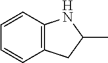 | 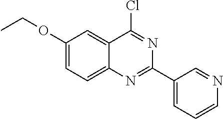 [SMILES: CCOc1ccc2nc(-c3cccnc3)nc(Cl)c2c1] | 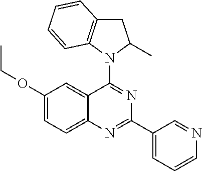 [SMILES: CCOc1ccc2nc(-c3cccnc3)nc(N3c4ccccc4CC3C)c2c1] | | 1H NMR (400 MHz, DMSO) δ 1.35 (t, J = 6.9 Hz, 3H), 1.49 (d, J = 6.1 Hz, 3H), 2.92 (dd, J = 15.4, 6.1 Hz, 1H), 3.56- 3.39 (m, 1H), 4.07-3.94 (m, 2H), 5.31-5.21 (m, 1H), 6.93 (d, | DMSO | >98 | Method C1, E, F3, G2 | | | |
| | | | | | J = 7.9 Hz, 1H), | | | | | | |
| | | | | | 7.01 (td, J = 7.4, | | | | | | |
| | | | | | 1.0 Hz, 1H), 7.13 | | | | | | |
| | | | | | (td, J = 7.7, 1.4 Hz, | | | | | | |
| | | | | | 1H), 7.28 (d, J = | | | | | | |
| | | | | | 2.7 Hz, 1H), 7.37 | | | | | | |
| | | | | | (dd, J = 7.4, | | | | | | |
| | | | | | 1.3 Hz, 1H), 7.69 | | | | | | |
| | | | | | (dd, J = 9.2, 2.7 | | | | | | |
| | | | | | Hz, 1H), 8.10 (d, | | | | | | |
| | | | | | J = 9.2 Hz, 1H), | | | | | | |
| | | | | | 8.17 (ddd, J = | | | | | | |
| | | | | | 8.2, 5.6, 0.8 Hz, | | | | | | |
| | | | | | 1H), 9.03 (dd, | | | | | | |
| | | | | | J = 5.6, 1.4 Hz, | | | | | | |
| | | | | | 1H), 9.34 (dt, J = | | | | | | |
| | | | | | 8.3, 1.7 Hz, 1H), | | | | | | |
| | | | | | 9.62 (d, J = | | | | | | |
| | | | | | 1.9 Hz, 1H). | | | | | | |
| 681 | 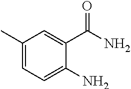 | 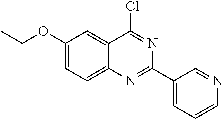 [SMILES: CCOc1ccc2nc(-c3cccnc3)nc(Cl)c2c1] | 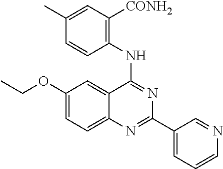 [SMILES: CCOc1ccc2nc(-c3cccnc3)nc(Nc3ccc(C)cc3C(N)=O)c2c1] | | 1H NMR (400 MHz, DMSO) δ 1.45 (t, J = 6.9 Hz, 3H), 2.37 (s, 3H), 4.16 (q, J = 6.9 Hz, 2H), 7.63- 7.35 (m, 3H), 8.07-7.84 (m, 3H), 8.41 (s, 1H), 8.70 (d, J = 8.4 Hz, 1H), 9.00 (dd, | DMSO | >98 | G1 without HCl | | | |
| | | | | | J = 5.4, 1.5 Hz, | | | | | | |
| | | | | | 1H), 9.00 (dt, J = | | | | | | |
| | | | | | 8.1, 1.8 Hz, 1H), | | | | | | |
| | | | | | 9.54-9.39 (m, | | | | | | |
| | | | | | 1H), 12.85 (s, | | | | | | |
| | | | | | 1H). | | | | | | |

TABLE 22

| | | | | | | | Puri- | Method | | | |
|---|---|---|---|---|---|---|---|---|---|---|---|
| | | | | | | ¹H | ty | of | | Reten- | LCMS |
| Num- | Starting | Starting | | Salt | | NMR | per- | Cou- | | tion | Meth- |
| ber | Material 1 | Material 2 | Product | type | ¹H NMR | Solvent | cent | pling | LCMS | Time | od |
| 682 | 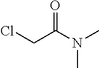 | 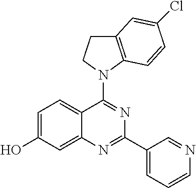 [SMILES: Oc1ccc2c(N3CCc4cc(Cl)ccc43)nc(-c3cccnc3)nc2c1] | 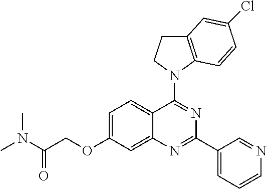 [SMILES: CN(C)C(=O)COc1ccc2c(N3CCc4cc(Cl)ccc43)nc(-c3cccnc3)nc2c1] | | 1H NMR (400 MHz, CDCl3) δ 3.05 (s, 3H), 3.14 (s, 3H), 3.23 (t, J = 7.9 Hz, 2H), 4.51 (t, J = 8.0 Hz, 2H), 4.90 (s, 2H), 7.13 (dd, J = 8.6, 2.2 Hz, 1H), 7.21 (dd, J = 9.2, 2.6 Hz, 1H), 7.26 (s, 1H), 7.30 (d, | CDCl3 | >98 | Method N | | | |
| | | | | | J = 6.1 Hz, 2H), | | | | | | |
| | | | | | 7.41 (ddd, J = | | | | | | |
| | | | | | 8.0, 4.8, 0.9 Hz, | | | | | | |
| | | | | | 1H), 7.95 (d, J = | | | | | | |
| | | | | | 9.2 Hz, 1H), 8.79- | | | | | | |
| | | | | | 8.64 (m, 2H), | | | | | | |
| | | | | | 9.69 (dd, J = | | | | | | |
| | | | | | 2.2, 0.9 Hz, 1H). | | | | | | |
| 683 | 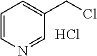 | 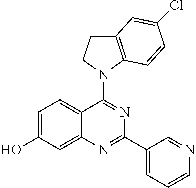 [SMILES: Oc1ccc2c(N3CCc4cc(Cl)ccc43)nc(-c3cccnc3)nc2c1] | 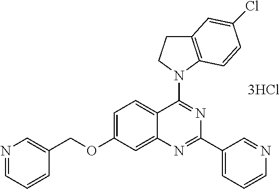 [SMILES: Clc1ccc2c(c1)CCN2c1nc(-c2cccnc2)nc2cc(OCc3cccnc3)ccc12] | 3HCl | 1H NMR (400 MHz, DMSO) δ 4.65 (t, J = 7.8 Hz, 2H), 5.58 (s, 2H), 7.34 (dd, J = 8.7, 2.3 Hz, 1H), 7.40 (dd, J = 9.3, 2.6 Hz, 1H), 7.47 (d, J = 2.2 Hz, 1H), 7.67 (s, 1H), 7.84 (d, J = 8.6 Hz, 1H), 8.00 (dd, J = | DMSO | >98 | Method N | | | |
| | | | | | 8.0, 5.4 Hz, 2H), | | | | | | |
| | | | | | 8.27 (d, J = 9.3 | | | | | | |
| | | | | | Hz, 1H), 8.57 (d, | | | | | | |
| | | | | | J = 8.0 Hz, 1H), | | | | | | |
| | | | | | 8.88 (dd, J = | | | | | | |
| | | | | | 5.4, 1.4 Hz, 1H), | | | | | | |
| | | | | | 8.96 (dd, J = | | | | | | |
| | | | | | 5.3, 1.5 Hz, 1H), | | | | | | |
| | | | | | 9.15-9.05 (m, | | | | | | |
| | | | | | 2H), 9.57 (dd, J = | | | | | | |
| | | | | | 2.0, 0.8 Hz, 1H), | | | | | | |
| | | | | | 3.36-3.17 (m, | | | | | | |
| | | | | | 2H). | | | | | | |
| 684 | 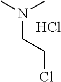 | 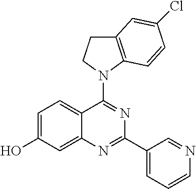 [SMILES: Oc1ccc2c(N3CCc4cc(Cl)ccc43)nc(-c3cccnc3)nc2c1] | 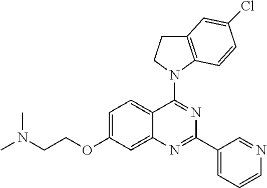 [SMILES: CN(C)CCOc1ccc2c(N3CCc4cc(Cl)ccc43)nc(-c3cccnc3)nc2c1] | | 1H NMR (400 MHz, DMSO) δ 2.73 (dd, J = 6.2, 4.9 Hz, 2H), 3.23 (t, J = 7.9 Hz, 2H), 4.29 (t, J = 5.7 Hz, 2H), 4.56 (t, J = 8.0 Hz, 2H), 7.22-7.09 (m, 1H), 7.29 (dd, J = 8.6, 2.3 Hz, 1H), 7.39 (dd, J = | DMSO | >98 | Method N | | | |
| | | | | | 19.2, 2.5 Hz, | | | | | | |
| | | | | | 2H), 7.62-7.50 (m, | | | | | | |
| | | | | | 1H), 7.66 (d, J = | | | | | | |
| | | | | | 8.6 Hz, 1H), 8.09 | | | | | | |
| | | | | | (d, J = 9.3 Hz, | | | | | | |
| | | | | | 1H), 8.78-8.62 | | | | | | |
| | | | | | (m, 2H), 9.61- | | | | | | |
| | | | | | 9.50 (m, 1H), 2.56- | | | | | | |
| | | | | | 2.43 (m, 6H). | | | | | | |
| 685 | 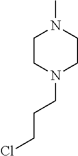 | 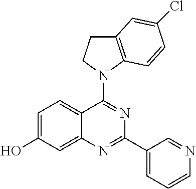 [SMILES: Oc1ccc2c(N3CCc4cc(Cl)ccc43)nc(-c3cccnc3)nc2c1] | 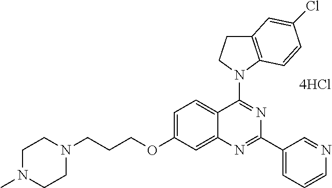 [SMILES: CN1CCN(CCCOc2ccc3c(N4CCc5cc(Cl)ccc54)nc(-c4cccnc4)nc3c2)CC1] | 4HCl | 1H NMR (400 MHz, CDCl3) δ 2.12- 2.02 (m, 2H), 2.31 (s, 3H), 2.76- 2.36 (m, 10H), 3.23 (t, J = 8.0 Hz, 2H), 4.23 (t, J = 6.3 Hz, 2H), 4.50 (t, J = 8.0 Hz, 2H), 1.95- 1.65 (m, 2H), 7.09 (ddd, J = 17.2, | CDCl3 | >98 | Method N | | | |
| | | | | | 8.9, 2.4 Hz, 2H), | | | | | | |
| | | | | | 7.26-7.22 (m, | | | | | | |
| | | | | | 1H), 7.35 (d, J = | | | | | | |
| | | | | | 2.5 Hz, 1H), 7.41 | | | | | | |
| | | | | | (ddd, J = 8.0, | | | | | | |
| | | | | | 4.8, 0.9 Hz, 1H), | | | | | | |
| | | | | | 7.90 (d, J = 9.2 | | | | | | |
| | | | | | Hz, 1H), 8.69 (dd, | | | | | | |
| | | | | | J = 4.8, 1.7 Hz, | | | | | | |
| | | | | | 1H), 8.75 (dt, J = | | | | | | |
| | | | | | 8.0, 1.9 Hz, 1H), | | | | | | |
| | | | | | 9.70 (dd, J = 2.1, | | | | | | |
| | | | | | 0.9 Hz, 1H). | | | | | | |
| 686 | 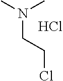 | 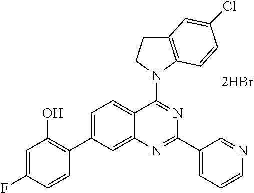 [SMILES: Oc1cc(F)ccc1-c1ccc2c(N3CCc4cc(Cl)ccc43)nc(-c3cccnc3)nc2c1] | 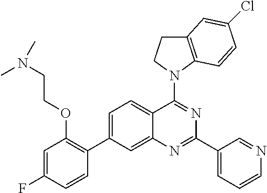 [SMILES: CN(C)CCOc1cc(F)ccc1-c1ccc2c(N3CCc4cc(Cl)ccc43)nc(-c3cccnc3)nc2c1] | | 1H NMR (400 MHz, CDCl3) δ 1.49- 1.39 (m, 2H), 3.44-2.88 (m, 8H), 4.10-3.96 (m, 2H), 4.57- 4.42 (m, 2H), 1.66-1.56 (m, 7H), 4.82 (s, 1H), 6.60 (dd, J = 8.8, 2.7 Hz, 1H), 6.77 (d, J = 2.6 Hz, 1H), | CDCl3 | >98 | Method N | | | |
| | | | | | 7.50-7.20 (m, | | | | | | |
| | | | | | 7H), 7.98-7.87 | | | | | | |
| | | | | | (m, 1H), 8.66 (dt, | | | | | | |
| | | | | | J = 4.8, 1.8 Hz, | | | | | | |
| | | | | | 1H), 8.77 (dt, J = | | | | | | |
| | | | | | 7.9, 2.0 Hz, 1H), | | | | | | |
| | | | | | 9.71 (dt, J = 2.4, | | | | | | |
| | | | | | 1.2 Hz, 1H). | | | | | | |

TABLE 23

| | | | | | | | Puri- | Method | | | |
|---|---|---|---|---|---|---|---|---|---|---|---|
| | | | | | | ¹H | ty | of | | Reten- | LCMS |
| Num- | Starting | Starting | | Salt | | NMR | per- | Cou- | | tion | Meth- |
| ber | Material 1 | Material 2 | Product | type | ¹H NMR | Solvent | cent | pling | LCMS | Time | od |
| 687 | 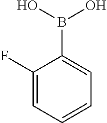 | 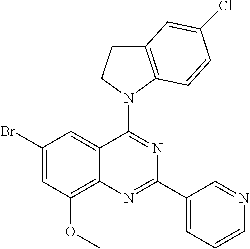 [SMILES: COc1cc(Br)cc2c(N3CCc4cc(Cl)ccc43)nc(-c3cccnc3)nc12] | 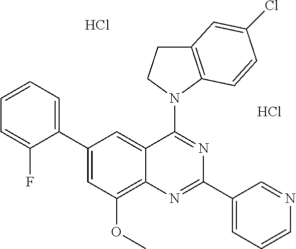 [SMILES: COc1cc(-c2ccccc2F)cc2c(N3CCc4cc(Cl)ccc43)nc(-c3cccnc3)nc12 ; Cl ; Cl] | 2 HCl | 1H NMR (400 MHz, CDCl3) δ 3.23 (t, J = 7.9 Hz, 2H), 4.16 (s, 3H), 4.56 (t, J = 8.0 Hz, 2H), 7.29- 7.10 (m, 4H), 7.55-7.34 (m, 5H), 7.75 (t, J = 1.4 Hz, 1H), 8.70 (dd, J = 4.9, 1.7 Hz, 1H), 8.86 (dt, J - 8.0, 1.9 Hz, 1H), 9.74 (dd, J = 2.1, 1.0 Hz, 1H). | DMSO | >98 | Method L | | | |
| 688 | 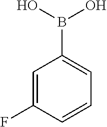 | 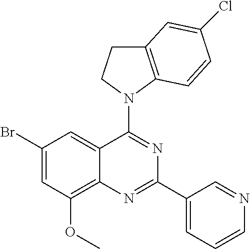 [SMILES: COc1cc(Br)cc2c(N3CCc4cc(Cl)ccc43)nc(-c3cccnc3)nc12] | 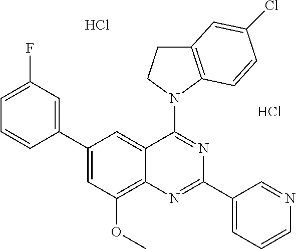 [SMILES: COc1cc(-c2cccc(F)c2)cc2c(N3CCc4cc(Cl)ccc43)nc(-c3cccnc3)nc12 ; Cl ; Cl] | 2 HCl | 1H NMR (400 MHz, CDCl3) δ 3.26 (t, J = 8.0 Hz, 2H), 4.19 (s, 3H), 4.57 (t, J = 8.0 Hz, 2H), 7.18- 7.05 (m, 2H), 7.52-7.24 (m, 7H), 7.77-7.69 (m, 1H), 8.70 (dd, J = 4.9, 1.7 Hz, 1H), 8.84 (dt, J = 7.9, 2.0 Hz, 1H), 9.79- 9.71 (m, 1H). | DMSO | >98 | Method L | | | |
| 689 | 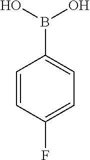 | 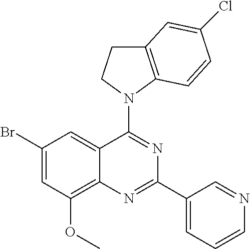 [SMILES: COc1cc(Br)cc2c(N3CCc4cc(Cl)ccc43)nc(-c3cccnc3)nc12] | 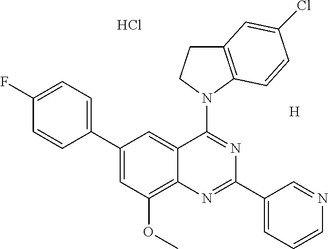 | 2 HCl | 1H NMR (400 MHz, CDCl3) δ 3.24 (t, J = 7.6 Hz, 2H), 4.16 (d, J = 2.3 Hz, 3H), 4.56 (td, J = 8.1, 2.4 Hz, 2H), 7.31- 7.08 (m, 5H), 7.51- 7.31 (m, 4H), 7.75 (dt, J = 2.7, 1.4 Hz, 1H), 8.70 (ddd, J = 4.8, 2.8, 1.6 Hz, 1H), 8.84 (ddd, J = 10.1, 5.2, 2.0 Hz, 1H), 9.74 (t, J = | DMSO | >98 | Method L | | | |
| | | | | | 2.4 Hz, 1H). | | | | | | |
| 690 | 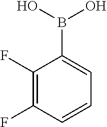 [SMILES: OB(O)c1cccc(F)c1F] | 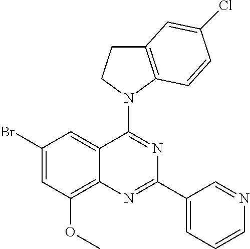 [SMILES: COc1cc(Br)cc2c(N3CCc4cc(Cl)ccc43)nc(-c3cccnc3)nc12] | 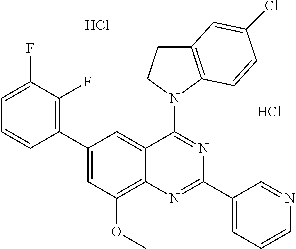 [SMILES: COc1cc(-c2cccc(F)c2F)cc2c(N3CCc4cc(Cl)ccc43)nc(-c3cccnc3)nc12 ; Cl ; Cl] | 2 HCl | 1H NMR (400 MHz, DMSO) δ 3.23 (t, J = 7.8 Hz, 2H), 4.10 (s, 3H), 4.63 (t, J = 7.9 Hz, 2H), 7.42- 7.26 (m, 2H), 7.44 (d, J = 2.2 Hz, 1H), 7.62-7.47 (m, 3H), 7.78 (d, J = 8.6 Hz, 1H), 7.88 (t, J = 1.4 Hz, 1H), 7.97 (dd, J = 8.1, 5.3 Hz, 1H), 8.91 (dd, J = 5.3, 1.6 Hz, | DMSO | >98 | Method L | | | |
| | | | | | 1H), 9.08 (dt, J = | | | | | | |
| | | | | | 8.0, 1.9 Hz, 1H), | | | | | | |
| | | | | | 9.56 (d, J = | | | | | | |
| | | | | | 2.0 Hz, 1H). | | | | | | |

TABLE 24

| | | | | | | | Puri- | Method | | | |
|---|---|---|---|---|---|---|---|---|---|---|---|
| | | | | | | ¹H | ty | of | | Reten- | LCMS |
| Num- | Starting | Starting | | Salt | | NMR | per- | Cou- | | tion | Meth- |
| ber | Material 1 | Material 2 | Product | type | ¹H NMR | Solvent | cent | pling | LCMS | Time | od |
| 691 | 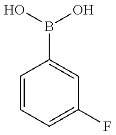 | 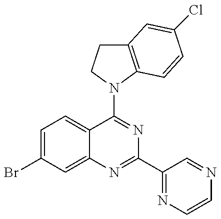 [SMILES: Clc1ccc2c(c1)CCN2c1nc(-c2cnccn2)nc2cc(Br)ccc12] | 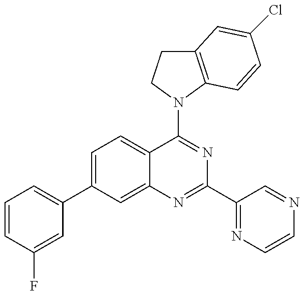 [SMILES: Fc1cccc(-c2ccc3c(N4CCc5cc(Cl)ccc54)nc(-c4cnccn4)nc3c2)c1] | | 1H NMR (400 MHz, CDCl3) δ 3.29 (t, J = 7.9 Hz, 2H), 4.62 (t, J = 8.0 Hz, 2H), 7.21-7.08 (m, 2H), 7.30 (dd, J = 2.2, 1.2 Hz, 1H), 7.60-7.41 (m, 4H), 7.75 (dd, J = 8.7, 2.0 Hz, 1H), 8.16 (d, J = 8.7 Hz, 1H), 8.53-8.40 (m, 1H), 8.69 (d, J = 2.4 Hz, 1H), 8.84 (dd, J = 2.5, 1.5 Hz, 1H), 9.76 (d, J = 1.5 Hz, 1H). | DMSO | >98 | Method L | | | |
| 692 | 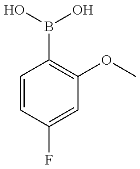 | 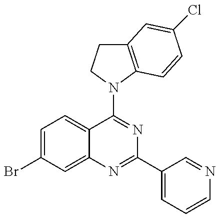 [SMILES: Clc1ccc2c(c1)CCN2c1nc(-c2cccnc2)nc2cc(Br)ccc12] | 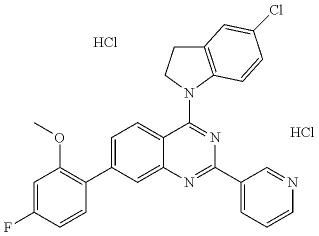 [SMILES: COc1cc(F)ccc1-c1ccc2c(N3CCc4cc(Cl)ccc43)nc(-c3cccnc3)nc2c1 ; Cl ; Cl] | 2HCl | 1H NMR (400 MHz, DMSO) δ 3.25 (s, 1H), 3.87 (s, 6H), 4.73 (t, J = 7.9 Hz, 4H), 6.97 (td, J = 8.4, 2.5 Hz, 2H), 7.16 (dd, J = 11.4, 2.5 Hz, 2H), 7.37 (dd, J = 8.7, 2.3 Hz, 2H), 7.49 (d, J = 2.2 Hz, 2H), 7.55 (dd, J = 8.5, 6.8 Hz, 2H), 7.80 (dd, J = 8.8, 1.9 Hz, 2H), 7.98 (d, J = 8.7 Hz, 2H), 8.05 (dd, J = 8.1, 5.4 Hz, 2H), 8.18 (s, 2H), 8.33 (d, J = 8.8 Hz, 2H), 8.99 (dd, J = 5.3, 1.5 Hz, 2H), 9.18 (dt, J = 8.2, 1.8 Hz, 2H), 9.59 (dt, J = 1.9 Hz, 2H). | DMSO | >98 | Method L | | | |
| 693 | 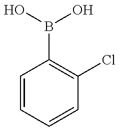 | 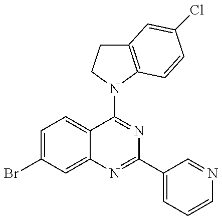 [SMILES: Clc1ccc2c(c1)CCN2c1nc(-c2cccnc2)nc2cc(Br)ccc12] | 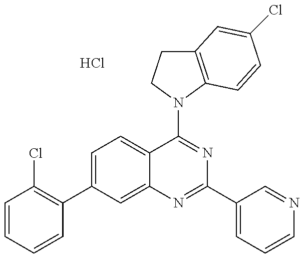 [SMILES: Clc1ccc2c(c1)CCN2c1nc(-c2cccnc2)nc2cc(-c3ccccc3Cl)ccc12 ; Cl] | HCl | 1H NMR (400 MHz, DMSO) δ 4.72 (t, J = 7.9 Hz, 2H), 7.42-7.32 (m, 1H), 7.77-7.44 (m, 6H), 7.98-7.84 (m, 2H), 8.03 (d, J = 1.8 Hz, 1H), 8.38 (d, J = 8.7 Hz, 1H), 8.87 (dd, J = 5.1, 1.6 Hz, 1H), 9.00 (dt, J = 8.2, 1.9 Hz, 1H), 9.58 (dd, J = 2.1, 0.9 Hz, 1H), 3.32-3.22 (m, 2H). | DMSO | >98 | Method L | | | |
| 694 | 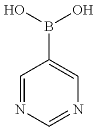 | 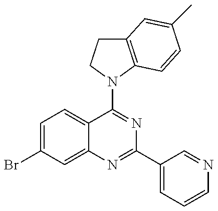 [SMILES: Cc1ccc2c(c1)CCN2c1nc(-c2cccnc2)nc2cc(Br)ccc12] | 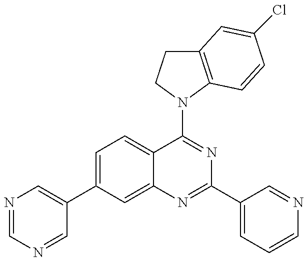 [SMILES: Clc1ccc2c(c1)CCN2c1nc(-c2cccnc2)nc2cc(-c3cncnc3)ccc12] | | 1H NMR (400 MHz, DMSO) δ 2.34 (s, 3H), 3.21 (t, J = 7.7 Hz, 2H), 4.64 (t, J = 7.9 Hz, 2H), 7.11 (d, J = 8.1 Hz, 1H), 7.21 (s, 1H), 7.59 (dd, J = 7.8, 4.9 Hz, 1H), 7.75 (d, J = 8.2 Hz, 1H), 8.01 (dd, J = 8.8, 2.0 Hz, 1H), 8.46-8.30 (m, 2H), 8.80-8.68 (m, 2H), 9.30 (s, 1H), 9.39 (s, 2H), 9.59 (d, J = 1.9 Hz, 1H). | DMSO | >98 | Method L | | | |
| 695 | 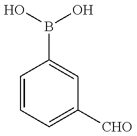 [SMILES: O=Cc1cccc(B(O)O)c1] | 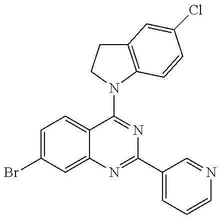 [SMILES: Clc1ccc2c(c1)CCN2c1nc(-c2cccnc2)nc2cc(Br)ccc12] | 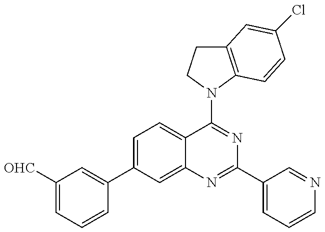 [SMILES: O=Cc1cccc(-c2ccc3c(N4CCc5cc(Cl)ccc54)nc(-c4cccnc4)nc3c2)c1] | | 1H NMR (400 MHz, DMSO) δ 3.26 (t, J = 8.0 Hz, 2H), 4.66 (t, J = 8.0 Hz, 2H), 7.33 (dd, J = 8.6, 2.3 Hz, 1H), 7.44 (d, J = 2.3 Hz, 1H), 7.67-7.49 (m, 1H), 7.85-7.72 (m, 2H), 8.09-7.93 (m, 2H), 8.40-8.23 (m, 3H), 8.46 (t, J = 1.7 Hz, 1H), 8.79- 8.67 (m, 2H), 9.58 (dd, J = 2.0, 0.9 Hz, 1H), 10.16 (s, 1H). | DMSO | >98 | Method L | | | |
| 696 | 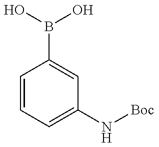 [SMILES: CC(C)(C)OC(=O)Nc1cccc(B(O)O)c1] | 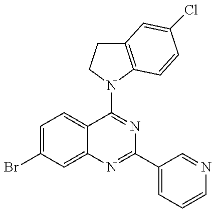 [SMILES: Clc1ccc2c(c1)CCN2c1nc(-c2cccnc2)nc2cc(Br)ccc12] | 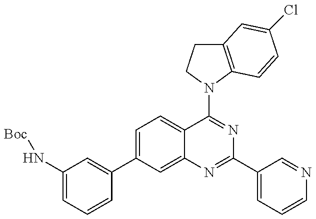 [SMILES: CC(C)(C)OC(=O)Nc1cccc(-c2ccc3c(N4CCc5cc(Cl)ccc54)nc(-c4cccnc4)nc3c2)c1] | | 1H NMR (400 MHz, DMSO) δ 1.52 (s, 9H), 3.26 (t, J = 7.8 Hz, 2H), 4.66 (t, J = 8.1 Hz, 2H), 7.64-7.26 (m, 5H), 7.92-7.77 (m, 2H), 8.16-8.00 (m, 2H), 8.31 (d, J = 8.8 Hz, 1H), 8.79-8.64 (m, 2H), 9.61-9.47 (m, 2H). | DMSO | >98 | Method L | | | |
| 697 | 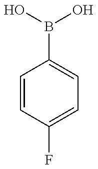 | 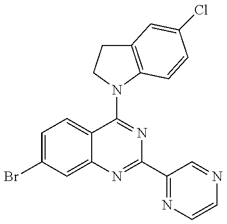 [SMILES: Clc1ccc2c(c1)CCN2c1nc(-c2cnccn2)nc2cc(Br)ccc12] | 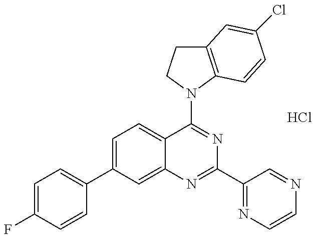 [SMILES: Fc1ccc(-c2ccc3c(N4CCc5cc(Cl)ccc54)nc(-c4cnccn4)nc3c2)cc1 ; Cl] | HCl | 1H NMR (400 MHz, DMSO) δ 3.31(t, J = 7.6 Hz, 2H), 4.84 (t, J = 7.6 Hz, 2H), 7.53-7.37 (m, 3H), 7.55 (d, J = 2.3 Hz, 1H), 8.01-7.90 (m, 2H), 8.07 (dd, J = 8.9, 2.0 Hz, 1H), 8.26 (d, J = 8.7 Hz, 1H), 8.58-8.45 (m, 2H), 9.05-8.93 (m, 2H), 9.63-9.54 (m, 1H). | DMSO | >98 | Method L | | | |
| 698 | 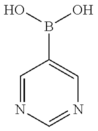 | 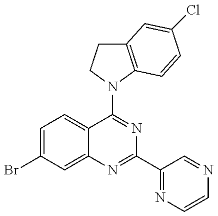 [SMILES: Clc1ccc2c(c1)CCN2c1nc(-c2cnccn2)nc2cc(Br)ccc12] | 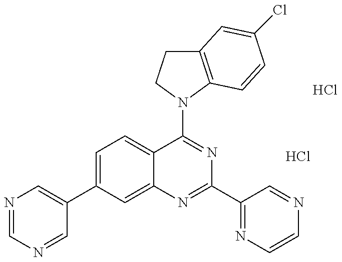 [SMILES: Clc1ccc2c(c1)CCN2c1nc(-c2cnccn2)nc2cc(-c3cncnc3)ccc12 ; Cl ; Cl] | 2HCl | 1H NMR (400 MHz, DMSO) δ 4.87 (t, J = 7.6 Hz, 2H), 7.50 (dd, J = 8.7, 2.3 Hz, 1H), 7.56 (d, J = 2.2 Hz, 1H), 8.36-8.17 (m, 2H), 8.57 (d, J = 8.9 Hz, 1H), 8.66 (d, J = 1.9 Hz, 1H), 9.05-8.94 (m, 2H), 9.40-9.31 (m, 3H), 9.57 (d, J = 1.4 Hz, 1H), 3.35-3.26 (m, 2H). | DMSO | >98 | Method L | | | |
| 699 | 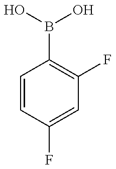 [SMILES: OB(O)c1ccc(F)cc1F] | 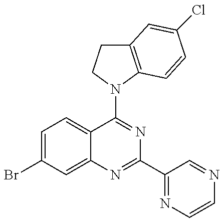 [SMILES: Clc1ccc2c(c1)CCN2c1nc(-c2cnccn2)nc2cc(Br)ccc12] | 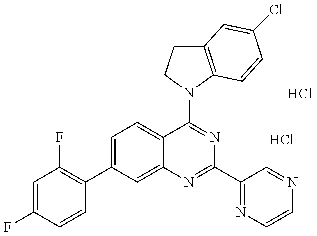 [SMILES: Fc1ccc(-c2ccc3c(N4CCc5cc(Cl)ccc54)nc(-c4cnccn4)nc3c2)c(F)c1 ; Cl ; Cl] | 2HCl | 1H NMR (400 MHz, DMSO) δ 4.84 (t, J = 7.7 Hz, 2H), 7.40-7.28 (m, 1H), 7.59-7.44 (m, 3H), 7.83 (td, J = 8.9, 6.5 Hz, 1H), 7.92 (dt, J = 8.8, 1.7 Hz, 1H), 8.27 (d, J = 8.7 Hz, 1H), 8.40 (s, 1H), 8.51 (d, J = 8.9 Hz, 1H), 9.03-8.89 (m, 2H), 9.57 (d, J = 1.1 Hz, 1H), 3.34-3.25 (m, 2H). | DMSO | >98 | Method L | | | |
| 700 | 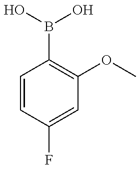 | 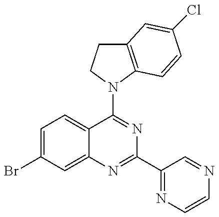 [SMILES: Clc1ccc2c(c1)CCN2c1nc(-c2cnccn2)nc2cc(Br)ccc12] | 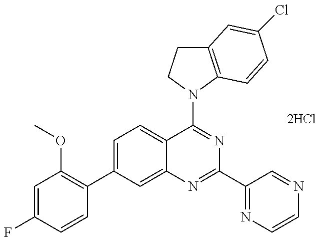 [SMILES: COc1cc(F)ccc1-c1ccc2c(N3CCc4cc(Cl)ccc43)nc(-c3cnccn3)nc2c1] | 2HCl | 1H NMR (400 MHz, DMSO) δ 3.90 (s, 3H), 4.85 (t, J = 7.7 Hz, 2H), 7.00 (td, J = 8.4, 2.4 Hz, 1H), 7.18 (dd, J = 11.5, 2.5 Hz, 1H), 3.33-3.27 (m, 2H), 7.64-7.45 (m, 3H), 7.89 (dd, J = 8.8, 1.9 Hz, 1H), 8.27 (d, J = 8.6 Hz, 1H), 8.54-8.34 (m, 2H), 9.05-8.92 (m, 2H), 9.58 (d, J = 1.4 Hz, 1H). | DMSO | >98 | Method L | | | |
| 701 | 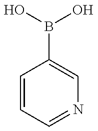 | 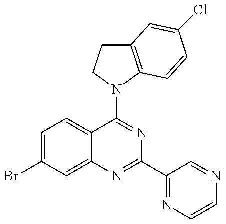 [SMILES: Clc1ccc2c(c1)CCN2c1nc(-c2cnccn2)nc2cc(Br)ccc12] | 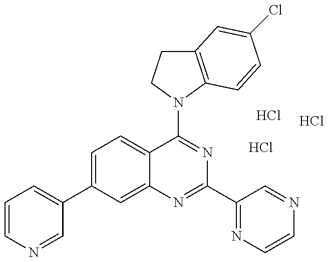 [SMILES: Clc1ccc2c(c1)CCN2c1nc(-c2cnccn2)nc2cc(-c3cccnc3)ccc12 ; Cl ; Cl ; Cl] | 3HCl | 1H NMR (400 MHz, DMSO) δ 4.85 (t, J = 7.7 Hz, 2H), 7.48 (dd, J = 8.7, 2.3 Hz, 1H), 7.55 (d, J = 2.2 Hz, 1H), 8.02-7.92 (m, 1H), 8.19 (dd, J = 8.8, 2.0 Hz, 1H), 8.27 (d, J = 8.7 Hz, 1H), 8.56 (d, J = 8.9 Hz, 1H), 8.65 (d, J = 2.0 Hz, 1H), 8.79- 8.68 (m, 1H), 8.90 (dd, J = 5.2, 1.4 Hz, 1H), 8.97 (s, 2H), 9.32 (d, J = 2.2 Hz, 1H), 9.59 (t, J = 1.4 Hz, 1H), 3.37-3.26 (m, 2H). | DMSO | >98 | Method L | | | |
| 702 | 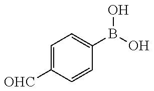 [SMILES: O=Cc1ccc(B(O)O)cc1] | 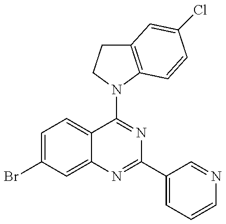 [SMILES: Clc1ccc2c(c1)CCN2c1nc(-c2cccnc2)nc2cc(Br)ccc12] | 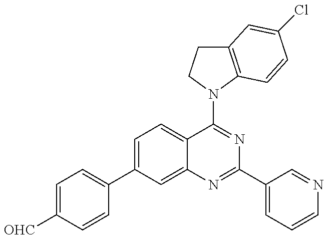 [SMILES: O=Cc1ccc(-c2ccc3c(N4CCc5cc(Cl)ccc54)nc(-c4cccnc4)nc3c2)cc1] | | 1H NMR (400 MHz, DMSO) δ 3.26 (t, J = 8.1Hz, 2H), 4.67 (t, J = 8.0 Hz, 2H), 7.34 (dd, J = 8.6, 2.3 Hz, 1H), 7.45 (d, J = 2.2 Hz, 1H), 7.66-7.54 (m, 1H), 7.82 (d, J = 8.7 Hz, 1H), 8.04-7.92 (m, 1H), 8.12-8.04 (m, 2H), 8.24-8.12 (m, 2H), 8.41-8.29 (m, 2H), 8.78-8.64 (m, 2H), 9.58 (d, J = 1.9 Hz, 1H), 10.12 (s, 1H). | DMSO | >98 | Method L | | | |

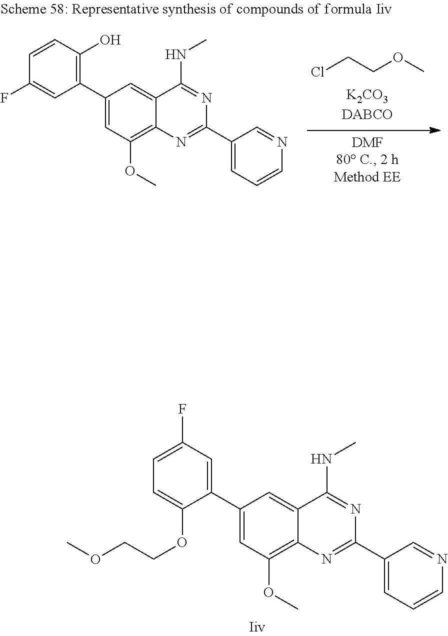

### Method EE: 6-(5-fluoro-2-(2-methoxyethoxy)phenyl)-8-methoxy-N-methyl-2-(pyridin-3-yl)quinazolin-4-amine

A mixture of 4-fluoro-2-(8-methoxy-4-(methylamino)-2-(pyridin-3-yl)quinazolin-6-yl)phenol (0.25 g, 0.664 mmol), 2-Chloroethyl methyl ether (0.303 ml, 3.32 mmol), K₂CO₃ (0.459 g, 3.32 mmol) and DABCO (0.037 g, 0.332 mmol) in DMF (10 ml) was heated at 80° C. for 2 h. After cooling to r.t., the reaction mixture was diluted with water (50 mL). The resultant precipitate was collected by filtration and dried. The obtained solid was purified by silicagel column chromatography (Hex:E.A.=1:1 to 0:1) to give 0.23 g of the product. The obtained free base was converted to the HCl salt by adding 1 M HCl-EtOH. The HCl salt was crystallized from IPA to give 186 mg of the product as a yellow powder in a 55% yield. The 1H NMR of this compound was consistent with the desired product. ¹H NMR (400 MHz, DMSO) δ 9.56 (d, J=1.9 Hz, 1H), 9.18 (d, J=8.0 Hz, 2H), 8.98 (d, J=5.3 Hz, 1H), 8.12-8.00 (m, 2H), 7.77 (d, J=1.6 Hz, 1H), 7.44 (dd, J=9.4, 3.1 Hz, 1H), 7.30-7.17 (m, 2H), 4.21-4.15 (m, 2H), 4.05 (s, 3H), 3.68-3.63 (m, 2H), 3.25 (s, 3H), 3.22 (d, J=4.2 Hz, 3H).

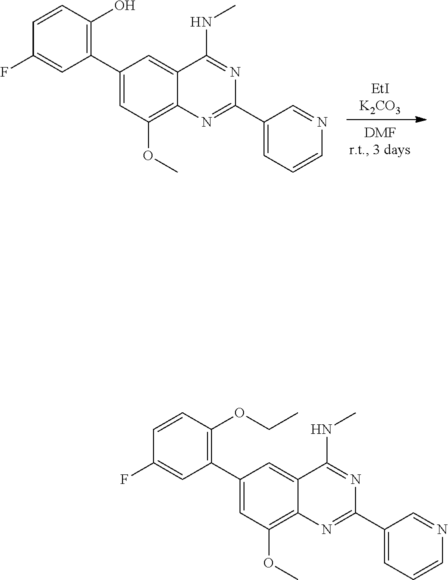 [SMILES: CNc1nc(-c2cccnc2)nc2c(OC)cc(-c3cc(F)ccc3O)cc12 ; CCOc1ccc(F)cc1-c1cc(OC)c2nc(-c3cccnc3)nc(NC)c2c1]

### Method EE: 6-(2-ethoxy-5-fluorophenyl)-8-methoxy-N-methyl-2-(pyridin-3-yl)quinazolin-4-amine

A mixture of 4-fluoro-2-(8-methoxy-4-(methylamino)-2-(pyridin-3-yl)quinazolin-6-yl)phenol (0.25 g, 0.664 mmol), Ethyliodide (0.106 ml, 1.328 mmol) and K2CO3 (0.184 g, 1.328 mmol) in DMF (5 ml) was stirred for 3 days at r.t. The reaction mixture was diluted with water (10 mL) and extracted with AcOEt (10 mL×2). The combined organic layers were washed with water (20 mL) and brine (15 mL) and dried over MgSO4. It was filtered off and the filtrate was concentrated in vacuo. The resultant residue was purified by silicagel column chromatography (Hex:E.A.=1:1 to 0:1) to give 0.13 g of the product. The obtained free base was converted to the HCl salt by adding 1N HCl-EtOH. The HCl salt was crystallized from IPA/H2O to give 102 mg of the product as a pale brown solid in a 32% yield. The 1H NMR of this compound was consistent with the desired product. ¹H NMR (400 MHz, DMSO) δ 9.56 (s, 1H), 9.26-9.08 (m, 2H), 8.96 (d, J=5.3 Hz, 1H), 8.09-8.05 (m, 1H), 8.05-7.98 (m, 1H), 7.70 (d, J=1.6 Hz, 1H), 7.41 (dd, J=9.4, 3.1 Hz, 1H), 7.29-7.22 (m, 1H), 7.21-7.13 (m, 1H), 4.08 (q, J=6.9 Hz, 2H), 4.04 (s, 3H), 3.21 (d, J=4.3 Hz, 3H), 1.31 (t, J=6.9 Hz, 3H).

TABLE 25

| | Starting | Starting | | Salt | | ¹H MNR | Purity | Method | | Retention | LCMS |
|---|---|---|---|---|---|---|---|---|---|---|---|
| Number | Material 1 | Material 2 | Product | type | ¹H NMR | Solvent | percent | of Coupling | LCMS | Time | Method |
| 703 | 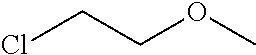 | 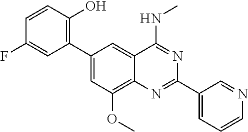 [SMILES: CNc1nc(-c2cccnc2)nc2c(OC)cc(-c3cc(F)ccc3O)cc12] | 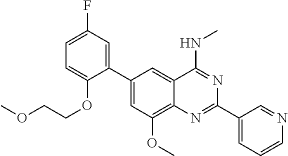 [SMILES: CNc1nc(-c2cccnc2)nc2c(OC)cc(-c3cc(F)ccc3OCCOC)cc12] | 2HCl | 1H NMR (400 MHz, DMSO) δ 9.56 (d, J = 1.9 Hz, 1H), 9.18 (d, J = 8.0 Hz, 2H), 8.98 (d, J = 5.3 Hz, 1H), 8.12-8.00 (m, 2H), 7.77 (d, J = 1.6 Hz, 1H), 7.44 (dd, J = 9.4, | DMSO | >98 | EE(K2CO3, DABCO at 80° C.) | | | |
| | | | | | 3.1 Hz, 1H), 7.30- | | | | | | |
| | | | | | 7.17 (m, 2H), | | | | | | |
| | | | | | 4.21-4.15 (m, | | | | | | |
| | | | | | 2H), 4.05 (s, 3H), | | | | | | |
| | | | | | 3.68-3.63 (m, | | | | | | |
| | | | | | 2H), 3.25 (s, 3H), | | | | | | |
| | | | | | 3.22 (d, J = | | | | | | |
| | | | | | 4.2 Hz, 3H). | | | | | | |
| 704 | Ethyl iodide | 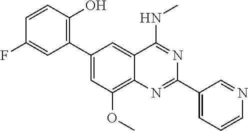 [SMILES: CNc1nc(-c2cccnc2)nc2c(OC)cc(-c3cc(F)ccc3O)cc12] | 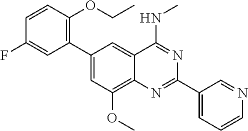 [SMILES: CCOc1ccc(F)cc1-c1cc(OC)c2nc(-c3cccnc3)nc(NC)c2c1] | 2HCl | 1H NMR (400 MHz, DMSO) δ 9.56 (s, 1H), 9.26-9.08 (m, 2H), 8.96 (d, J = 5.3 Hz, 1H), 8.09-8.05 (m, 1H), 8.05-7.98 (m, 1H), 7.70 (d, J = 1.6 Hz, 1H), | DMSO | >98 | EE(K2CO3 at r.t.) | | | |
| | | | | | 7.41 (dd, J = | | | | | | |
| | | | | | 9.4, 3.1 Hz, 1H), | | | | | | |
| | | | | | 7.29-7.22 (m, | | | | | | |
| | | | | | 1H), 7.21-7.13 | | | | | | |
| | | | | | (m, 1H), 4.08 (q, | | | | | | |
| | | | | | J = 6.9 Hz, 2H), | | | | | | |
| | | | | | 4.04 (s, 3H), 3.21 | | | | | | |
| | | | | | (d, J = 4.3 Hz, | | | | | | |
| | | | | | 3H), 1.31 (t, J = | | | | | | |
| | | | | | 6.9 Hz, 3H). | | | | | | |

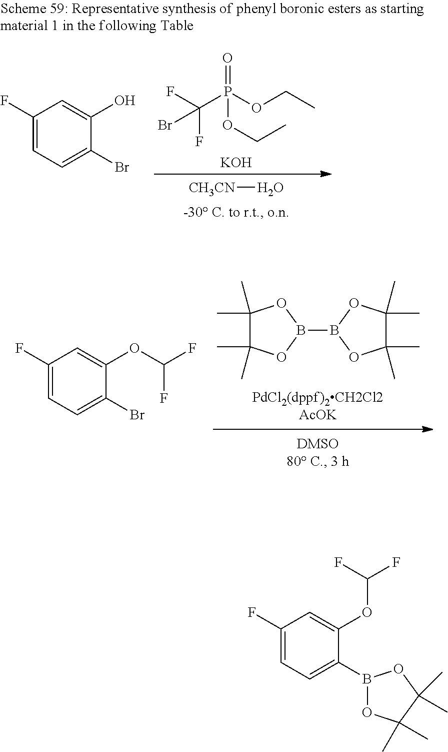

### 1-Bromo-2-(difluoromethoxy)-4-fluorobenzene

(Ref. Tetrahedron 65 (2009) 5278-5283) To a solution of 2-Bromo-5-fluorophenol (3.0 ml, 27.0 mmol) and KOH (15.13 g, 270 mmol) in CH3CN (25 ml) and Water (25 ml) was slowly added Bromodifluoromethyl diethylphosphonate (9.58 ml, 53.9 mmol) at −30° C. Then, the reaction mixture was stirred at r.t. o.n. The reaction mixture was diluted with water (30 mL) and extracted with AcOEt (30 mL×2). The combined organic layers were washed with brine (40 mL×1) and dried over MgSO4. It was filtered off and the filtrate was concentrated in vacuo. The resultant residue was purified by silica-gel column chromatography (Hex:E.A.=10:1 to 3:1) to give 5.63 g of the product as a colorless oil in an 87% yield. ¹H NMR (400 MHz, CDCl₃) δ 7.58 (dd, J=8.9, 5.9 Hz, 1H), 7.04-6.96 (m, 1H), 6.92-6.84 (m, 1H), 6.56 (t, J=72.8 Hz, 1H).

### 2-(2-(Difluoromethoxy)-4-fluorophenyl)-4,4,5,5-tetramethyl-1,3,2-dioxaborolane

A mixture of 1-bromo-2-(difluoromethoxy)-4-fluorobenzene (2.50 g, 10.37 mmol), Bis(pinacolato)diboron (3.95 g, 15.56 mmol), 1,1′-Bis(diphenylphosphino)ferrocene-palladium(11) dichloride dichloromethane complex (0.424 g, 0.519 mmol) and Potassium acetate (3.05 g, 31.1 mmol) in DMSO (40 ml) was heated at 80° C. for 4 h. After cooling to r.t., the reaction mixture was diluted with water (50 mL) and extracted with AcOEt (50 mL×2). The combined organic layers were washed with water (100 mL×1) and brine (100 mL×1) and dried over MgSO4. It was filtered off and the filtrate was concentrated in vacuo. The resultant residue was purified by silica-gel column chromatography (Hex:E.A.=9:1 to 4:1) to give 2.42 g of the product as a brown oil in an 81% yield. The 1H NMR of this product was consistent with the desired product. The 1H NMR of this compound was consistent with the desired product. ¹H NMR (400 MHz, CDCl₃) δ 7.75 (dd, J=8.4, 7.1 Hz, 1H), 6.99-6.93 (m, 1H), 6.89 (dd, J=9.8, 2.3 Hz, 1H), 6.55 (t, J=74.9 Hz, 1H), 1.34 (s, 12H).

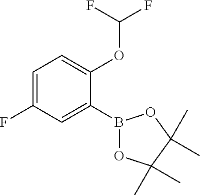 [SMILES: CC1(C)OB(c2cc(F)ccc2OC(F)F)OC1(C)C]

2-(2-(Difluoromethoxy)-5-fluorophenyl)-4,4,5,5-tetramethyl-1,3,2-dioxaborolane was also prepared in the same manner as above. ¹H NMR (400 MHz, CDCl₃) δ 7.45-7.34 (m, 1H), 7.15-7.01 (m, 2H), 6.47 (t, J=75.3 Hz, 1H), 1.35 (s, 12H).

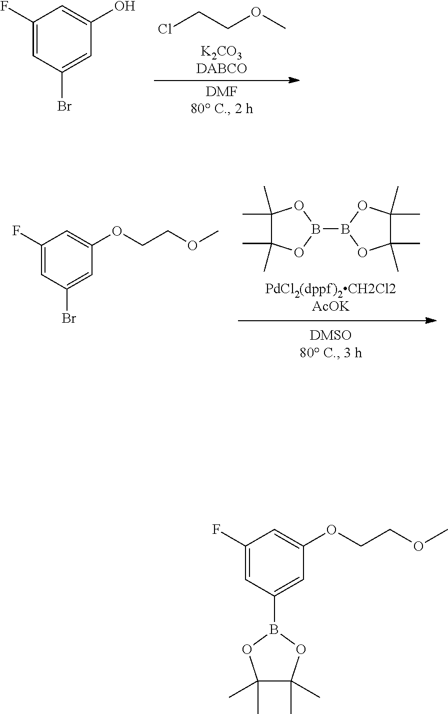

### 1-Bromo-3-fluoro-5-(2-methoxyethoxy)benzene

A mixture of 3-Bromo-5-fluorophenol (1.20 g, 6.28 mmol), 2-Chloroethyl methyl ether (2.87 ml, 31.4 mmol), K2CO3 (4.34 g, 31.4 mmol) and DABCO (0.352 g, 3.14 mmol) in DMF (15 mL) was heated at 80° C. for 2 h. After cooling to r.t., the reaction mixture was diluted with water (20 mL) and extracted with AcOEt (15 mL×2). The combined organic layers were washed with water (20 mL×1) and brine (20 mL×1) and dried over MgSO4. It was filtered off and the filtrate was concentrated in vacuo. The residue was purified by silicagel column chromatography (Hex:E.A.=10:1 to 3:1) to give 1.56 g of the product as a colorless oil in a quantitative yield. The 1H NMR of this compound was consistent with the desired product. ¹H NMR (400 MHz, CDCl₃) δ 6.90-6.87 (m, 1H), 6.87-6.82 (m, 1H), 6.62-6.57 (m, 1H), 4.11-4.05 (m, 2H), 3.76-3.70 (m, 2H), 3.44 (s, 3H).

### 2-(3-Fluoro-5-(2-methoxyethoxy)phenyl)-4,4,5,5-tetramethyl-1,3,2-dioxaborolane

A mixture of 1-bromo-3-fluoro-5-(2-methoxyethoxy)benzene (0.20 g, 0.803 mmol), Bis(pinacolato)diboron (0.245 g, 0.964 mmol), 1,1′-Bis(diphenylphosphino)ferrocenepalladium(11) dichloride, Toluene (0.029 g, 0.040 mmol) and Potassium acetate (0.151 ml, 2.409 mmol) in DMSO (5 ml) was heated at 80° C. for 3 h under N2. After cooling to r.t., the reaction mixture was diluted with water (20 mL) and extracted with AcOEt (15 mL×2). The combined organic layers were washed with water (15 mL×1) and brine (15 mL×1) and dried over MgSO4. It was filtered off and the filtrate was concentrated in vacuo to give 0.24 g of the product as a black oil in a quantitative yield. The 1H NMR of this compound was consistent with the desired product. ¹H NMR (400 MHz, CDCl₃) δ 7.12 (d, J=2.3 Hz, 1H), 7.09 (dd, J=8.2, 2.4 Hz, 1H), 6.77-6.71 (m, 1H), 4.16-4.12 (m, 2H), 3.76-3.72 (m, 2H), 3.45 (s, 3H), 1.33 (s, 12H).

TABLE 26

| | Starting | Starting | |
|---|---|---|---|
| Number | Material 1 | Material 2 | Product |
| 705 | 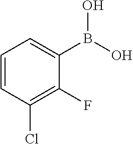 [SMILES: OB(O)c1cccc(Cl)c1F] | 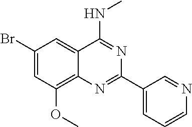 [SMILES: CNc1nc(-c2cccnc2)nc2c(OC)cc(Br)cc12] | 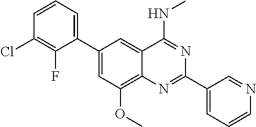 [SMILES: CNc1nc(-c2cccnc2)nc2c(OC)cc(-c3cccc(Cl)c3F)cc12] |
| 706 | 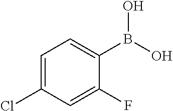 [SMILES: OB(O)c1ccc(Cl)cc1F] | 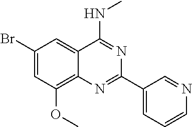 [SMILES: CNc1nc(-c2cccnc2)nc2c(OC)cc(Br)cc12] | 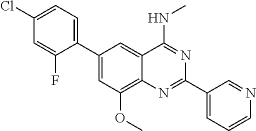 [SMILES: CNc1nc(-c2cccnc2)nc2c(OC)cc(-c3ccc(Cl)cc3F)cc12] |
| 707 | 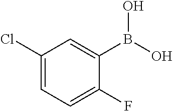 [SMILES: OB(O)c1cc(Cl)ccc1F] | 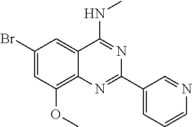 [SMILES: CNc1nc(-c2cccnc2)nc2c(OC)cc(Br)cc12] | 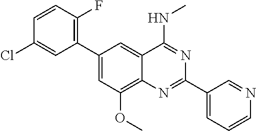 [SMILES: CNc1nc(-c2cccnc2)nc2c(OC)cc(-c3cc(Cl)ccc3F)cc12] |
| 708 | 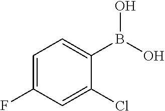 [SMILES: OB(O)c1ccc(F)cc1Cl] | 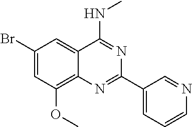 [SMILES: CNc1nc(-c2cccnc2)nc2c(OC)cc(Br)cc12] | 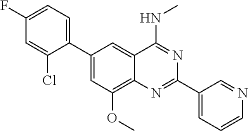 [SMILES: CNc1nc(-c2cccnc2)nc2c(OC)cc(-c3ccc(F)cc3Cl)cc12] |
| 709 | 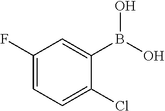 [SMILES: OB(O)c1cc(F)ccc1Cl] | 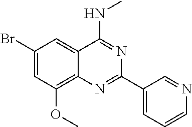 [SMILES: CNc1nc(-c2cccnc2)nc2c(OC)cc(Br)cc12] | 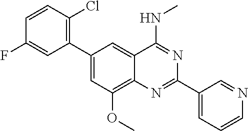 [SMILES: CNc1nc(-c2cccnc2)nc2c(OC)cc(-c3cc(F)ccc3Cl)cc12] |
| 710 | 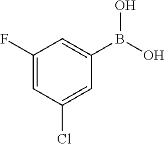 [SMILES: OB(O)c1cc(F)cc(Cl)c1] | 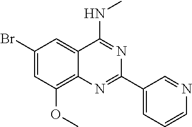 [SMILES: CNc1nc(-c2cccnc2)nc2c(OC)cc(Br)cc12] | 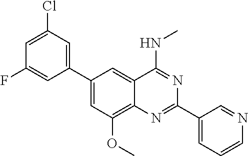 [SMILES: CNc1nc(-c2cccnc2)nc2c(OC)cc(-c3cc(F)cc(Cl)c3)cc12] |
| 711 | 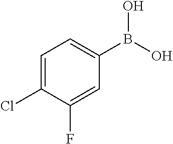 [SMILES: OB(O)c1ccc(Cl)c(F)c1] | 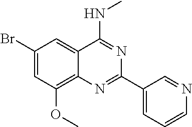 [SMILES: CNc1nc(-c2cccnc2)nc2c(OC)cc(Br)cc12] | 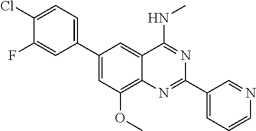 [SMILES: CNc1nc(-c2cccnc2)nc2c(OC)cc(-c3ccc(Cl)c(F)c3)cc12] |
| 712 | 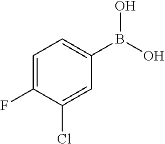 [SMILES: OB(O)c1ccc(F)c(Cl)c1] | 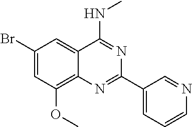 [SMILES: CNc1nc(-c2cccnc2)nc2c(OC)cc(Br)cc12] | 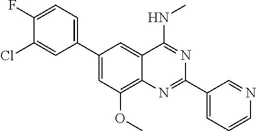 [SMILES: CNc1nc(-c2cccnc2)nc2c(OC)cc(-c3ccc(F)c(Cl)c3)cc12] |
| 713 | 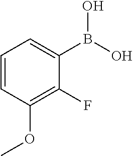 | 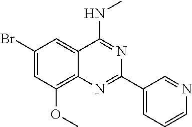 [SMILES: CNc1nc(-c2cccnc2)nc2c(OC)cc(Br)cc12] | 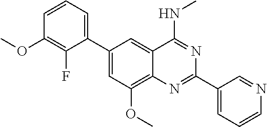 [SMILES: CNc1nc(-c2cccnc2)nc2c(OC)cc(-c3cccc(OC)c3F)cc12] |
| 714 | 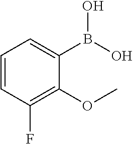 | 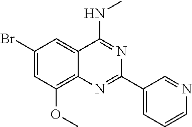 [SMILES: CNc1nc(-c2cccnc2)nc2c(OC)cc(Br)cc12] | 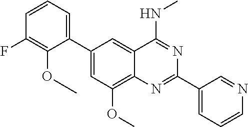 [SMILES: CNc1nc(-c2cccnc2)nc2c(OC)cc(-c3cccc(F)c3OC)cc12] |
| 715 | 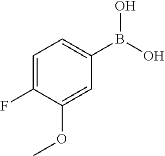 | 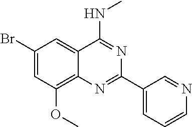 [SMILES: CNc1nc(-c2cccnc2)nc2c(OC)cc(Br)cc12] | 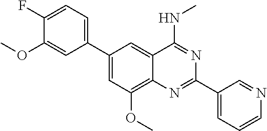 [SMILES: CNc1nc(-c2cccnc2)nc2c(OC)cc(-c3ccc(F)c(OC)c3)cc12] |
| 716 | 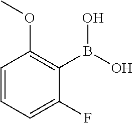 | 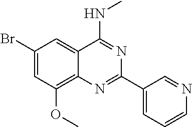 [SMILES: CNc1nc(-c2cccnc2)nc2c(OC)cc(Br)cc12] | 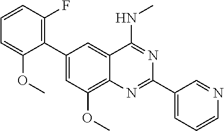 [SMILES: CNc1nc(-c2cccnc2)nc2c(OC)cc(-c3c(F)cccc3OC)cc12] |
| 717 | 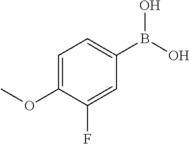 | 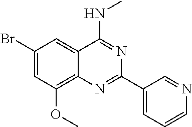 [SMILES: CNc1nc(-c2cccnc2)nc2c(OC)cc(Br)cc12] | 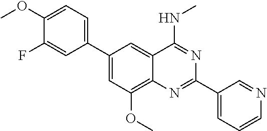 [SMILES: CNc1nc(-c2cccnc2)nc2c(OC)cc(-c3ccc(OC)c(F)c3)cc12] |
| 718 | 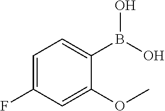 | 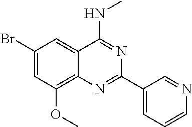 [SMILES: CNc1nc(-c2cccnc2)nc2c(OC)cc(Br)cc12] | 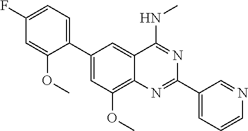 [SMILES: CNc1nc(-c2cccnc2)nc2c(OC)cc(-c3ccc(F)cc3OC)cc12] |
| 719 | 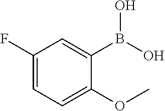 | 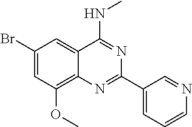 [SMILES: CNc1nc(-c2cccnc2)nc2c(OC)cc(Br)cc12] | 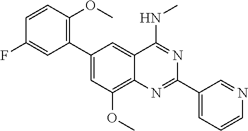 [SMILES: CNc1nc(-c2cccnc2)nc2c(OC)cc(-c3cc(F)ccc3OC)cc12] |
| 720 | 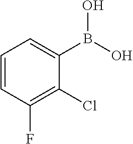 [SMILES: OB(O)c1cccc(F)c1Cl] | 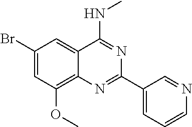 [SMILES: CNc1nc(-c2cccnc2)nc2c(OC)cc(Br)cc12] | 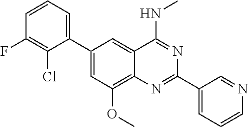 [SMILES: CNc1nc(-c2cccnc2)nc2c(OC)cc(-c3cccc(F)c3Cl)cc12] |
| 721 | 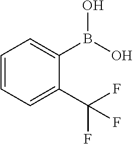 [SMILES: OB(O)c1ccccc1C(F)(F)F] | 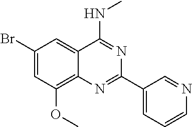 [SMILES: CNc1nc(-c2cccnc2)nc2c(OC)cc(Br)cc12] | 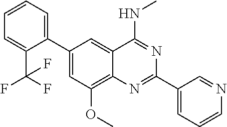 [SMILES: CNc1nc(-c2cccnc2)nc2c(OC)cc(-c3ccccc3C(F)(F)F)cc12] |
| 722 | 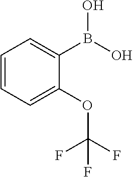 [SMILES: OB(O)c1ccccc1OC(F)(F)F] | 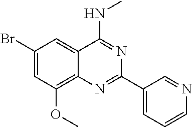 [SMILES: CNc1nc(-c2cccnc2)nc2c(OC)cc(Br)cc12] | 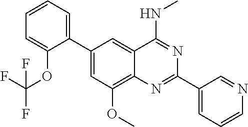 [SMILES: CNc1nc(-c2cccnc2)nc2c(OC)cc(-c3ccccc3OC(F)(F)F)cc12] |
| 723 | 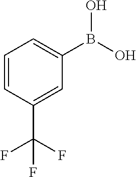 [SMILES: OB(O)c1cccc(C(F)(F)F)c1] | 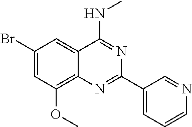 [SMILES: CNc1nc(-c2cccnc2)nc2c(OC)cc(Br)cc12] | 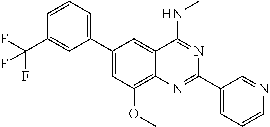 [SMILES: CNc1nc(-c2cccnc2)nc2c(OC)cc(-c3cccc(C(F)(F)F)c3)cc12] |
| 724 | 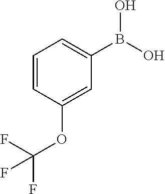 [SMILES: OB(O)c1cccc(OC(F)(F)F)c1] | 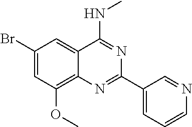 [SMILES: CNc1nc(-c2cccnc2)nc2c(OC)cc(Br)cc12] | 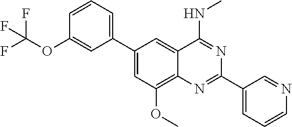 [SMILES: CNc1nc(-c2cccnc2)nc2c(OC)cc(-c3cccc(OC(F)(F)F)c3)cc12] |
| 725 | 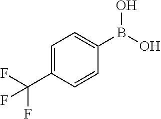 [SMILES: OB(O)c1ccc(C(F)(F)F)cc1] | 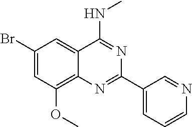 [SMILES: CNc1nc(-c2cccnc2)nc2c(OC)cc(Br)cc12] | 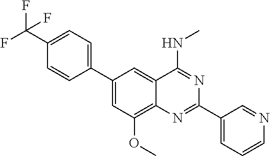 [SMILES: CNc1nc(-c2cccnc2)nc2c(OC)cc(-c3ccc(C(F)(F)F)cc3)cc12] |
| 726 | 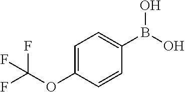 [SMILES: OB(O)c1ccc(OC(F)(F)F)cc1] | 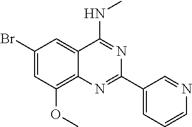 [SMILES: CNc1nc(-c2cccnc2)nc2c(OC)cc(Br)cc12] | 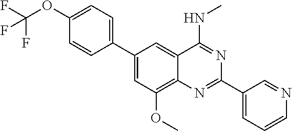 [SMILES: CNc1nc(-c2cccnc2)nc2c(OC)cc(-c3ccc(OC(F)(F)F)cc3)cc12] |
| 727 | 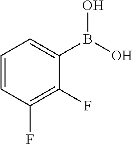 [SMILES: OB(O)c1cccc(F)c1F] | 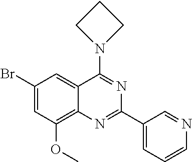 [SMILES: COc1cc(Br)cc2c(N3CCC3)nc(-c3cccnc3)nc12] | 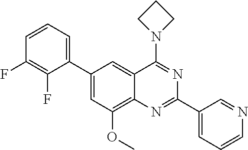 [SMILES: COc1cc(-c2cccc(F)c2F)cc2c(N3CCC3)nc(-c3cccnc3)nc12] |
| 728 | 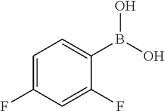 [SMILES: OB(O)c1ccc(F)cc1F] | 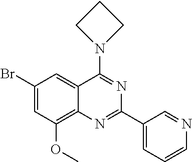 [SMILES: COc1cc(Br)cc2c(N3CCC3)nc(-c3cccnc3)nc12] | 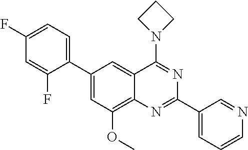 [SMILES: COc1cc(-c2ccc(F)cc2F)cc2c(N3CCC3)nc(-c3cccnc3)nc12] |
| 729 | 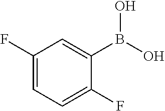 [SMILES: OB(O)c1cc(F)ccc1F] | 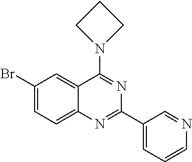 [SMILES: Brc1ccc2nc(-c3cccnc3)nc(N3CCC3)c2c1] | 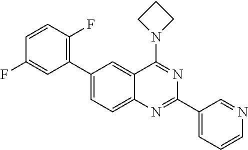 [SMILES: Fc1ccc(F)c(-c2ccc3nc(-c4cccnc4)nc(N4CCC4)c3c2)c1] |
| 730 | 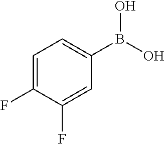 [SMILES: OB(O)c1ccc(F)c(F)c1] | 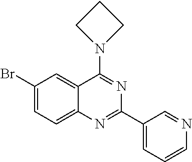 [SMILES: Brc1ccc2nc(-c3cccnc3)nc(N3CCC3)c2c1] | 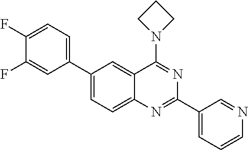 [SMILES: Fc1ccc(-c2ccc3nc(-c4cccnc4)nc(N4CCC4)c3c2)cc1F] |
| 731 | 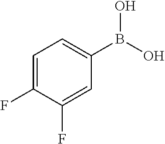 [SMILES: OB(O)c1ccc(F)c(F)c1] | 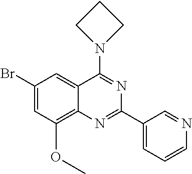 [SMILES: COc1cc(Br)cc2c(N3CCC3)nc(-c3cccnc3)nc12] | 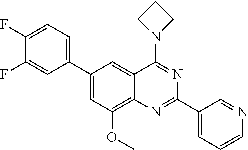 [SMILES: COc1cc(-c2ccc(F)c(F)c2)cc2c(N3CCC3)nc(-c3cccnc3)nc12] |
| 732 | 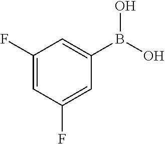 [SMILES: OB(O)c1cc(F)cc(F)c1] | 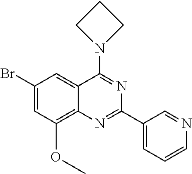 [SMILES: COc1cc(Br)cc2c(N3CCC3)nc(-c3cccnc3)nc12] | 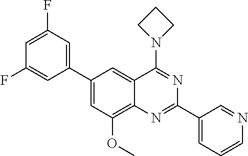 [SMILES: COc1cc(-c2cc(F)cc(F)c2)cc2c(N3CCC3)nc(-c3cccnc3)nc12] |
| 733 | 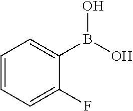 | 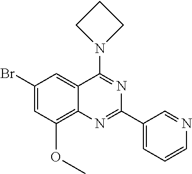 [SMILES: COc1cc(Br)cc2c(N3CCC3)nc(-c3cccnc3)nc12] | 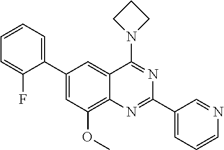 [SMILES: COc1cc(-c2ccccc2F)cc2c(N3CCC3)nc(-c3cccnc3)nc12] |
| 734 | 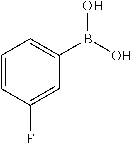 | 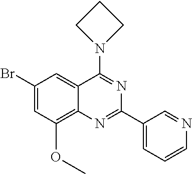 [SMILES: COc1cc(Br)cc2c(N3CCC3)nc(-c3cccnc3)nc12] | 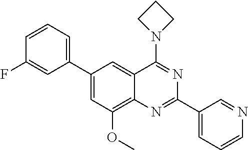 [SMILES: COc1cc(-c2cccc(F)c2)cc2c(N3CCC3)nc(-c3cccnc3)nc12] |
| 735 | 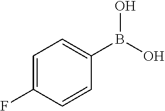 | 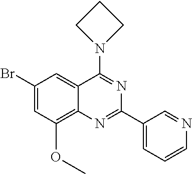 [SMILES: COc1cc(Br)cc2c(N3CCC3)nc(-c3cccnc3)nc12] | 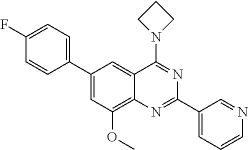 [SMILES: COc1cc(-c2ccc(F)cc2)cc2c(N3CCC3)nc(-c3cccnc3)nc12] |
| 736 | 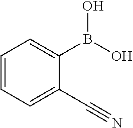 [SMILES: N#Cc1ccccc1B(O)O] | 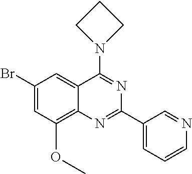 [SMILES: COc1cc(Br)cc2c(N3CCC3)nc(-c3cccnc3)nc12] | 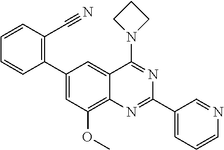 [SMILES: COc1cc(-c2ccccc2C#N)cc2c(N3CCC3)nc(-c3cccnc3)nc12] |
| 737 | 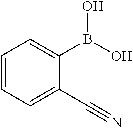 [SMILES: N#Cc1ccccc1B(O)O] | 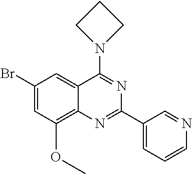 [SMILES: COc1cc(Br)cc2c(N3CCC3)nc(-c3cccnc3)nc12] | 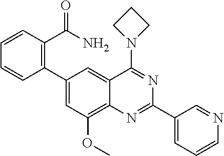 [SMILES: COc1cc(-c2ccccc2C(N)=O)cc2c(N3CCC3)nc(-c3cccnc3)nc12] |
| 738 | 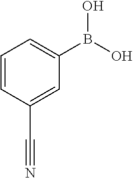 [SMILES: N#Cc1cccc(B(O)O)c1] | 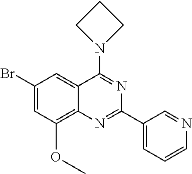 [SMILES: COc1cc(Br)cc2c(N3CCC3)nc(-c3cccnc3)nc12] | 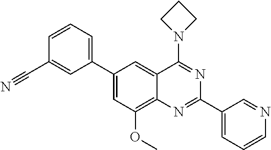 [SMILES: COc1cc(-c2cccc(C#N)c2)cc2c(N3CCC3)nc(-c3cccnc3)nc12] |
| 739 | 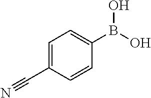 [SMILES: N#Cc1ccc(B(O)O)cc1] | 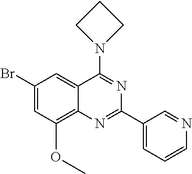 [SMILES: COc1cc(Br)cc2c(N3CCC3)nc(-c3cccnc3)nc12] | 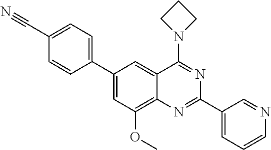 [SMILES: COc1cc(-c2ccc(C#N)cc2)cc2c(N3CCC3)nc(-c3cccnc3)nc12] |
| 740 | 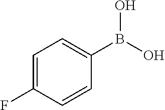 | 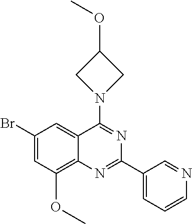 [SMILES: COc1cc(Br)cc2c(N3CC(OC)C3)nc(-c3cccnc3)nc12] | 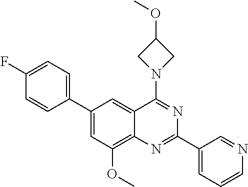 [SMILES: COc1cc(-c2ccc(F)cc2)cc2c(N3CC(OC)C3)nc(-c3cccnc3)nc12] |
| 741 | 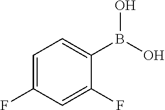 [SMILES: OB(O)c1ccc(F)cc1F] | 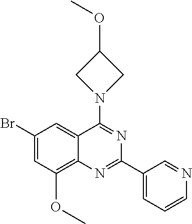 [SMILES: COc1cc(Br)cc2c(N3CC(OC)C3)nc(-c3cccnc3)nc12] | 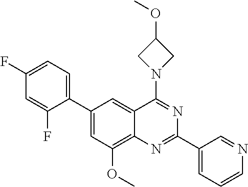 [SMILES: COc1cc(-c2ccc(F)cc2F)cc2c(N3CC(OC)C3)nc(-c3cccnc3)nc12] |
| 742 | 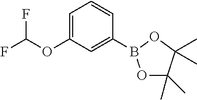 [SMILES: CC1(C)OB(c2cccc(OC(F)F)c2)OC1(C)C] | 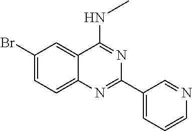 [SMILES: CNc1nc(-c2cccnc2)nc2ccc(Br)cc12] | 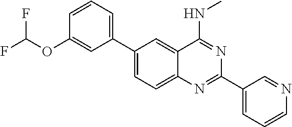 [SMILES: CNc1nc(-c2cccnc2)nc2ccc(-c3cccc(OC(F)F)c3)cc12] |
| 743 | 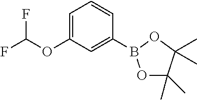 [SMILES: CC1(C)OB(c2cccc(OC(F)F)c2)OC1(C)C] | 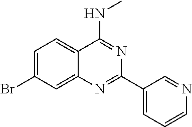 [SMILES: CNc1nc(-c2cccnc2)nc2cc(Br)ccc12] | 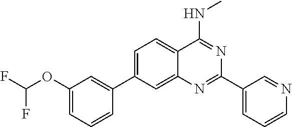 [SMILES: CNc1nc(-c2cccnc2)nc2cc(-c3cccc(OC(F)F)c3)ccc12] |
| 744 | 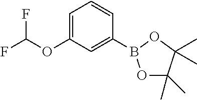 [SMILES: CC1(C)OB(c2cccc(OC(F)F)c2)OC1(C)C] | 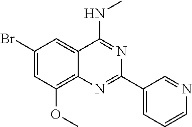 [SMILES: CNc1nc(-c2cccnc2)nc2c(OC)cc(Br)cc12] | 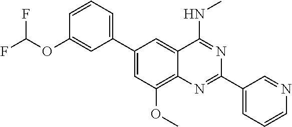 [SMILES: CNc1nc(-c2cccnc2)nc2c(OC)cc(-c3cccc(OC(F)F)c3)cc12] |
| 745 | 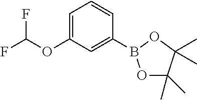 [SMILES: CC1(C)OB(c2cccc(OC(F)F)c2)OC1(C)C] | 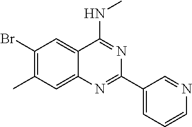 [SMILES: CNc1nc(-c2cccnc2)nc2cc(C)c(Br)cc12] | 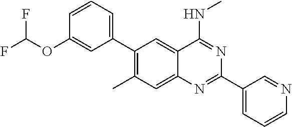 [SMILES: CNc1nc(-c2cccnc2)nc2cc(C)c(-c3cccc(OC(F)F)c3)cc12] |
| 746 | 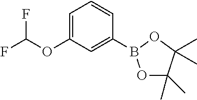 [SMILES: CC1(C)OB(c2cccc(OC(F)F)c2)OC1(C)C] | 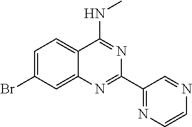 [SMILES: CNc1nc(-c2cnccn2)nc2cc(Br)ccc12] | 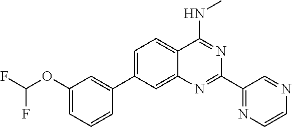 [SMILES: CNc1nc(-c2cnccn2)nc2cc(-c3cccc(OC(F)F)c3)ccc12] |
| 747 | 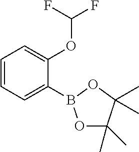 [SMILES: CC1(C)OB(c2ccccc2OC(F)F)OC1(C)C] | 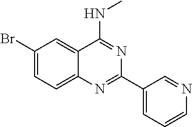 [SMILES: CNc1nc(-c2cccnc2)nc2ccc(Br)cc12] | 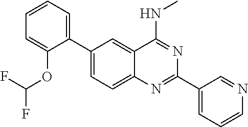 [SMILES: CNc1nc(-c2cccnc2)nc2ccc(-c3ccccc3OC(F)F)cc12] |
| 748 | 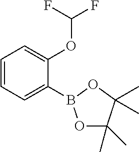 [SMILES: CC1(C)OB(c2ccccc2OC(F)F)OC1(C)C] | 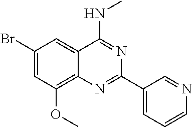 [SMILES: CNc1nc(-c2cccnc2)nc2c(OC)cc(Br)cc12] | 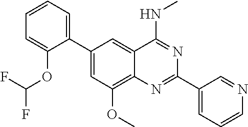 [SMILES: CNc1nc(-c2cccnc2)nc2c(OC)cc(-c3ccccc3OC(F)F)cc12] |
| 749 | 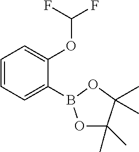 [SMILES: CC1(C)OB(c2ccccc2OC(F)F)OC1(C)C] | 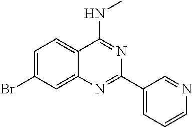 [SMILES: CNc1nc(-c2cccnc2)nc2cc(Br)ccc12] | 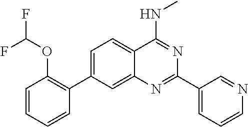 [SMILES: CNc1nc(-c2cccnc2)nc2cc(-c3ccccc3OC(F)F)ccc12] |
| 750 | 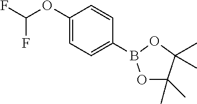 [SMILES: CC1(C)OB(c2ccc(OC(F)F)cc2)OC1(C)C] | 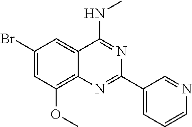 [SMILES: CNc1nc(-c2cccnc2)nc2c(OC)cc(Br)cc12] | 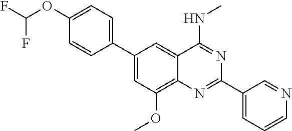 [SMILES: CNc1nc(-c2cccnc2)nc2c(OC)cc(-c3ccc(OC(F)F)cc3)cc12] |
| 751 | 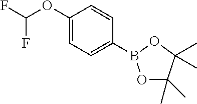 [SMILES: CC1(C)OB(c2ccc(OC(F)F)cc2)OC1(C)C] | 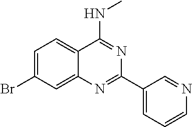 [SMILES: CNc1nc(-c2cccnc2)nc2cc(Br)ccc12] | 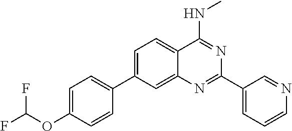 [SMILES: CNc1nc(-c2cccnc2)nc2cc(-c3ccc(OC(F)F)cc3)ccc12] |
| 752 | 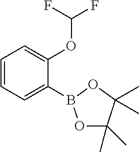 [SMILES: CC1(C)OB(c2ccccc2OC(F)F)OC1(C)C] | 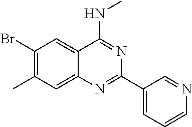 [SMILES: CNc1nc(-c2cccnc2)nc2cc(C)c(Br)cc12] | 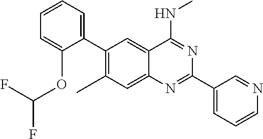 [SMILES: CNc1nc(-c2cccnc2)nc2cc(C)c(-c3ccccc3OC(F)F)cc12] |
| 753 | 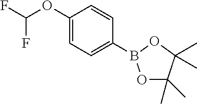 [SMILES: CC1(C)OB(c2ccc(OC(F)F)cc2)OC1(C)C] | 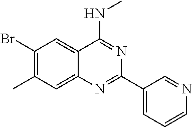 [SMILES: CNc1nc(-c2cccnc2)nc2cc(C)c(Br)cc12] | 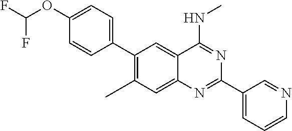 [SMILES: CNc1nc(-c2cccnc2)nc2cc(C)c(-c3ccc(OC(F)F)cc3)cc12] |
| 754 | 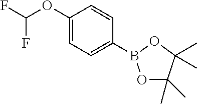 [SMILES: CC1(C)OB(c2ccc(OC(F)F)cc2)OC1(C)C] | 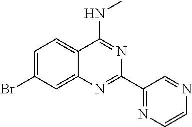 [SMILES: CNc1nc(-c2cnccn2)nc2cc(Br)ccc12] | 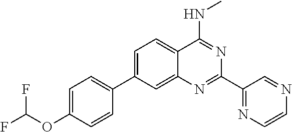 [SMILES: CNc1nc(-c2cnccn2)nc2cc(-c3ccc(OC(F)F)cc3)ccc12] |
| 755 | 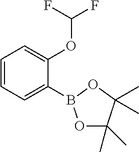 [SMILES: CC1(C)OB(c2ccccc2OC(F)F)OC1(C)C] | 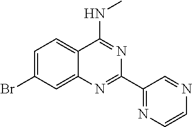 [SMILES: CNc1nc(-c2cnccn2)nc2cc(Br)ccc12] | 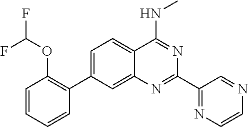 [SMILES: CNc1nc(-c2cnccn2)nc2cc(-c3ccccc3OC(F)F)ccc12] |
| 756 | 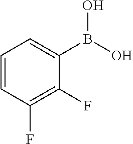 [SMILES: OB(O)c1cccc(F)c1F] | 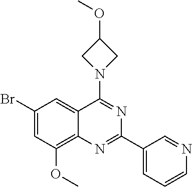 [SMILES: COc1cc(Br)cc2c(N3CC(OC)C3)nc(-c3cccnc3)nc12] | 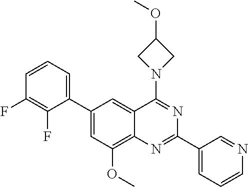 [SMILES: COc1cc(-c2cccc(F)c2F)cc2c(N3CC(OC)C3)nc(-c3cccnc3)nc12] |
| 757 | 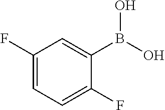 [SMILES: OB(O)c1cc(F)ccc1F] | 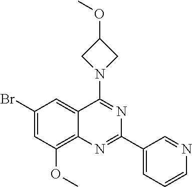 [SMILES: COc1cc(Br)cc2c(N3CC(OC)C3)nc(-c3cccnc3)nc12] | 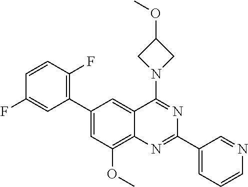 [SMILES: COc1cc(-c2cc(F)ccc2F)cc2c(N3CC(OC)C3)nc(-c3cccnc3)nc12] |
| 758 | 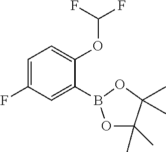 [SMILES: CC1(C)OB(c2cc(F)ccc2OC(F)F)OC1(C)C] | 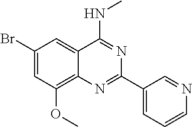 [SMILES: CNc1nc(-c2cccnc2)nc2c(OC)cc(Br)cc12] | 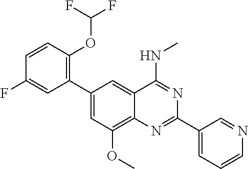 [SMILES: CNc1nc(-c2cccnc2)nc2c(OC)cc(-c3cc(F)ccc3OC(F)F)cc12] |
| 759 | 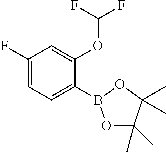 [SMILES: CC1(C)OB(c2ccc(F)cc2OC(F)F)OC1(C)C] | 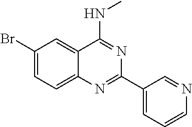 [SMILES: CNc1nc(-c2cccnc2)nc2ccc(Br)cc12] | 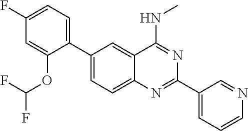 [SMILES: CNc1nc(-c2cccnc2)nc2ccc(-c3ccc(F)cc3OC(F)F)cc12] |
| 760 | 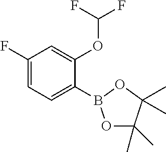 [SMILES: CC1(C)OB(c2ccc(F)cc2OC(F)F)OC1(C)C] | 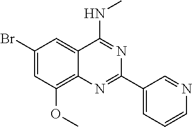 [SMILES: CNc1nc(-c2cccnc2)nc2c(OC)cc(Br)cc12] | 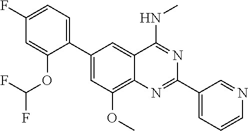 [SMILES: CNc1nc(-c2cccnc2)nc2c(OC)cc(-c3ccc(F)cc3OC(F)F)cc12] |
| 761 | 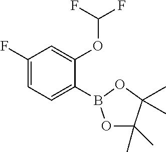 [SMILES: CC1(C)OB(c2ccc(F)cc2OC(F)F)OC1(C)C] | 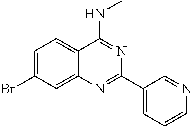 [SMILES: CNc1nc(-c2cccnc2)nc2cc(Br)ccc12] | 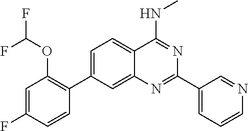 [SMILES: CNc1nc(-c2cccnc2)nc2cc(-c3ccc(F)cc3OC(F)F)ccc12] |
| 762 | 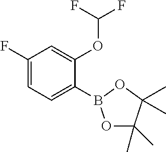 [SMILES: CC1(C)OB(c2ccc(F)cc2OC(F)F)OC1(C)C] | 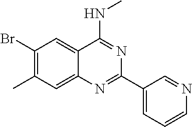 [SMILES: CNc1nc(-c2cccnc2)nc2cc(C)c(Br)cc12] | 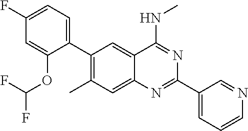 [SMILES: CNc1nc(-c2cccnc2)nc2cc(C)c(-c3ccc(F)cc3OC(F)F)cc12] |
| 763 | 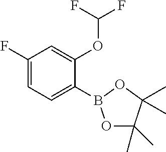 [SMILES: CC1(C)OB(c2ccc(F)cc2OC(F)F)OC1(C)C] | 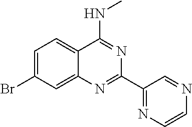 [SMILES: CNc1nc(-c2cnccn2)nc2cc(Br)ccc12] | 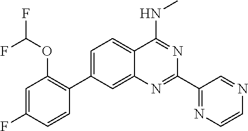 [SMILES: CNc1nc(-c2cnccn2)nc2cc(-c3ccc(F)cc3OC(F)F)ccc12] |
| 764 | 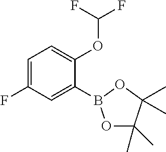 [SMILES: CC1(C)OB(c2cc(F)ccc2OC(F)F)OC1(C)C] | 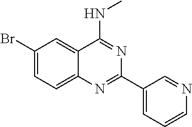 [SMILES: CNc1nc(-c2cccnc2)nc2ccc(Br)cc12] | 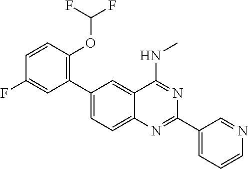 [SMILES: CNc1nc(-c2cccnc2)nc2ccc(-c3cc(F)ccc3OC(F)F)cc12] |
| 765 | 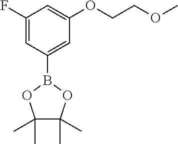 [SMILES: COCCOc1cc(F)cc(B2OC(C)(C)C(C)(C)O2)c1] | 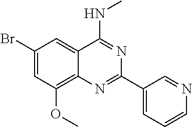 [SMILES: CNc1nc(-c2cccnc2)nc2c(OC)cc(Br)cc12] | 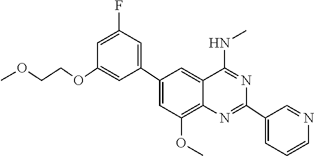 [SMILES: CNc1nc(-c2cccnc2)nc2c(OC)cc(-c3cc(F)cc(OCCOC)c3)cc12] |

| | Salt | | ¹H NMR | Purity | | | Retention | LCMS |
|---|---|---|---|---|---|---|---|---|
| Number | type | ¹H NMR | Solvent | percent | Method of Coupling | LCMS | Time | Method |
| 705 | 2HCl | 1H NMR (400 MHz, DMSO) δ 9.61 | DMSO | >98 | R3 | | | |
| | | (d, J = 1.9 Hz, 1H), 9.14-9.08 | | | at 80° C. | | | |
| | | (m, 1H), 8.99-8.86 (m, 2H), 8.11- | | | in | | | |
| | | 8.05 (m, 1H), 7.93 (dd, J = 8.1, | | | DME—EtOH—H₂O | | | |
| | | 5.3 Hz, 1H), 7.73-7.64 (m, 2H), | | | | | | |
| | | 7.55-7.50 (m, 1H), 7.45-7.38 | | | | | | |
| | | (m, 1H), 4.05 (s, 3H), 3.19 (d, J = | | | | | | |
| | | 4.4 Hz, 3H). | | | | | | |
| 706 | HCl | 1H NMR (400 MHz, DMSO) δ | DMSO | >98 | R3 | | | |
| | | 9.64-9.59 (m, 1H), 9.03-8.96 (m, | | | at 80° C. | | | |
| | | 1H), 8.86-8.75 (m, 2H), 8.06- | | | in | | | |
| | | 8.00 (m, 1H), 7.83-7.72 (m, 2H), | | | DME—EtOH—H₂O | | | |
| | | 7.68-7.61 (m, 1H), 7.52-7.42 | | | | | | |
| | | (m, 2H), 4.04 (s, 3H), 3.18 (d, J = | | | | | | |
| | | 4.4 Hz, 3H). | | | | | | |
| 707 | 2HCl | 1H NMR (400 MHz, DMSO) δ 9.61 | DMSO | >98 | R3 | | | |
| | | (d, J = 1.9 Hz, 1H), 9.16 (d, J = | | | at 80° C. | | | |
| | | 8.1 Hz, 1H), 9.03-8.89 (m, 2H), | | | in | | | |
| | | 8.11-8.05 (m, 1H), 7.98 (dd, J = | | | DME—EtOH—H₂O | | | |
| | | 8.1, 5.4 Hz, 1H), 7.81 (dd, J = 6.8, | | | | | | |
| | | 2.7 Hz, 1H), 7.61-7.52 (m, 2H), | | | | | | |
| | | 7.51-7.43 (m, 1H), 4.06 (s, 3H), | | | | | | |
| | | 3.20 (d, J = 4.4 Hz, 3H). | | | | | | |
| 708 | 2HCl | 1H NMR (400 MHz, DMSO) δ 9.58 | DMSO | >98 | R3 | | | |
| | | (d, J = 1.9 Hz, 1H), 9.32-9.12 | | | at 80° C. | | | |
| | | (m, 2H), 9.01 (dd, J = 5.5, 1.5 Hz, | | | in | | | |
| | | 1H), 8.11 (dd, J = 8.1, 5.5 Hz, | | | DME—EtOH—H₂O | | | |
| | | 1H), 8.04-7.96 (m, 1H), 7.69- | | | | | | |
| | | 7.61 (m, 2H), 7.50-7.36 (m, 2H), | | | | | | |
| | | 4.04 (s, 3H), 3.19 (d, J = 4.0 Hz, | | | | | | |
| | | 3H). | | | | | | |
| 709 | 2HCl | 1H NMR (400 MHz, DMSO) δ 9.60 | DMSO | >98 | R3 | | | |
| | | (d, J = 1.9 Hz, 1H), 9.26 (d, J = | | | at 80° C. | | | |
| | | 8.1 Hz, 1H), 9.11 (s, 1H), 9.00 (d, | | | in | | | |
| | | J = 5.3 Hz, 1H), 8.10 (dd, J = 8.0, | | | DME—EtOH—H₂O | | | |
| | | 5.6 Hz, 1H), 8.01 (d, J = 1.7 Hz, | | | | | | |
| | | 1H), 7.70 (dd, J = 8.9, 5.2 Hz, | | | | | | |
| | | 1H), 7.55-7.47 (m, 2H), 7.42- | | | | | | |
| | | 7.33 (m, 1H), 4.05 (s, 3H), 3.19 | | | | | | |
| | | (d, J = 4.2 Hz, 3H). | | | | | | |
| 710 | 2HCl | 1H NMR (400 MHz, DMSO) δ 9.54 | DMSO | >98 | R3 | | | |
| | | (s, 1H), 9.38 (s, 1H), 9.23 (d, J = | | | at 80° C. | | | |
| | | 8.1 Hz, 1H), 9.01 (dd, J = 5.5, 1.5 | | | in | | | |
| | | Hz, 1H), 8.34 (d, J = 1.9 Hz, 1H), | | | DME—EtOH—H₂O | | | |
| | | 8.10 (dd, J = 8.2, 5.5 Hz, 1H), | | | | | | |
| | | 7.94-7.90 (m, 1H), 7.90-7.81 | | | | | | |
| | | (m, 1H), 7.70 (s, 1H), 7.53-7.45 | | | | | | |
| | | (m, 1H), 4.12 (s, 3H), 3.21 (d, J = | | | | | | |
| | | 3.3 Hz, 3H). | | | | | | |
| 711 | 2HCl | 1H NMR (400 MHz, DMSO) δ 9.56 | DMSO | >98 | R3 | | | |
| | | (d, J = 2.0 Hz, 1H), 9.33 (s, 1H), | | | at 80° C. | | | |
| | | 9.21 (d, J = 7.6 Hz, 1H), 8.98 (dd, | | | in | | | |
| | | J = 5.5, 1.5 Hz, 1H), 8.35 (d, J = | | | DME—EtOH—H₂O | | | |
| | | 1.8 Hz, 1H), 8.10-8.01 (m, 2H), | | | | | | |
| | | 7.88-7.82 (m, 1H), 7.76 (t, J = | | | | | | |
| | | 8.1, 8.1 Hz, 1H), 7.71 (d, J = 1.7 | | | | | | |
| | | Hz, 1H), 4.12 (s, 3H), 3.22 (d, J = | | | | | | |
| | | 4.2 Hz, 3H). | | | | | | |
| 712 | 2HCl | 1H NMR (400 MHz, DMSO) δ 9.53 | DMSO | >98 | R3 | | | |
| | | (d, J = 2.0 Hz, 1H), 9.42 (s, 1H), | | | at 80° C. | | | |
| | | 9.20 (d, J = 8.1 Hz, 1H), 8.99 (dd, | | | in | | | |
| | | J = 5.6, 1.5 Hz, 1H), 8.30 (d, J = | | | DME—EtOH—H₂O | | | |
| | | 1.8 Hz, 1H), 8.17 (dd, J = 7.1, 2.3 | | | | | | |
| | | Hz, 1H), 8.08 (dd, J = 8.1, 5.4 Hz, | | | | | | |
| | | 1H), 8.01-7.93 (m, 1H), 7.67 (d, | | | | | | |
| | | J = 1.7 Hz, 1H), 7.58 (t, J = 9.0, | | | | | | |
| | | 9.0 Hz, 1H), 4.12 (s, 3H), 3.21 (d, | | | | | | |
| | | J = 4.0 Hz, 3H). | | | | | | |
| 713 | 2HCl | 1H NMR (400 MHz, DMSO) δ 9.59 | DMSO | >98 | R3 | | | |
| | | (d, J = 2.0 Hz, 1H), 9.17 (d, J = | | | | | | |
| | | 8.0 Hz, 1H), 9.08 (s, 1H), 8.95 | | | | | | |
| | | (dd, J = 5.4, 1.5 Hz, 1H), 8.10- | | | | | | |
| | | 8.05 (m, 1H), 8.01 (dd, J = 8.1, | | | | | | |
| | | 5.4 Hz, 1H), 7.55-7.50 (m, 1H), | | | | | | |
| | | 7.34-7.19 (m, 3H), 4.05 (s, 3H), | | | | | | |
| | | 3.92 (s, 3H), 3.20 (d, J = 4.3 Hz, | | | | | | |
| | | 3H). | | | | | | |
| 714 | 2HCl | 1H NMR (400 MHz, DMSO) δ 9.57 | DMSO | >98 | R3 | | | |
| | | (d, J = 1.9 Hz, 1H), 9.36-9.17 | | | | | | |
| | | (m, 2H), 9.00 (dd, J = 5.5, 1.5 Hz, | | | | | | |
| | | 1H), 8.13-8.04 (m, 2H), 7.56 (d, | | | | | | |
| | | J = 1.5 Hz, 1H), 7.43-7.34 (m, | | | | | | |
| | | 2H), 7.32-7.22 (m, 1H), 4.06 (s, | | | | | | |
| | | 3H), 3.76 (d, J = 1.3 Hz, 3H) (by | | | | | | |
| | | rotamer), 3.20 (d, J = 4.1 Hz, 3H). | | | | | | |
| 715 | 2HCl | 1H NMR (400 MHz, DMSO) δ 9.71 | DMSO | >98 | R3 | | | |
| | | (s, 1H), 9.54 (s, 1H), 9.17 (d, J = | | | | | | |
| | | 8.1 Hz, 1H), 8.99 (dd, J = 5.8, 1.4 | | | | | | |
| | | Hz, 1H), 8.44-8.36 (m, 1H), 8.06 | | | | | | |
| | | (dd, J = 8.1, 5.4 Hz, 1H), 7.74- | | | | | | |
| | | 7.65 (m, 2H), 7.53-7.45 (m, 1H), | | | | | | |
| | | 7.42-7.32 (m, 1H), 4.13 (s, 3H), | | | | | | |
| | | 4.04 (s, 3H), 3.23 (d, J = 3.9 Hz, | | | | | | |
| | | 3H). | | | | | | |
| 716 | 2HCl | 1H NMR (400 MHz, DMSO) δ 9.56 | DMSO | >98 | R3 | | | |
| | | (d, J = 1.9 Hz, 1H), 9.34-9.16 | | | | | | |
| | | (m, 2H), 9.02 (dt, J = 5.5, 1.6, 1.6 | | | | | | |
| | | Hz, 1H), 8.14-8.05 (m, 1H), 7.95 | | | | | | |
| | | (s, 1H), 7.53-7.43 (m, 1H), 7.43- | | | | | | |
| | | 7.37 (m, 1H), 7.06 (d, J = 8.6 | | | | | | |
| | | Hz, 1H), 7.03-6.95 (m, 1H), 4.01 | | | | | | |
| | | (s, 3H), 3.80 (s, 3H), 3.19 (d, J = | | | | | | |
| | | 4.0 Hz, 3H). | | | | | | |
| 717 | 2HCl | 1H NMR (400 MHz, DMSO) δ 9.61- | DMSO | >98 | R3 | | | |
| | | 9.41 (m, 2H), 9.16 (d, J = 7.9 | | | | | | |
| | | Hz, 1H), 8.99 (dd, J = 5.4, 1.5 Hz, | | | | | | |
| | | 1H), 8.31 (d, J = 1.8 Hz, 1H), 8.06 | | | | | | |
| | | (dd, J = 8.1, 5.4 Hz, 1H), 7.91- | | | | | | |
| | | 7.84 (m, 1H), 7.82-7.74 (m, 1H), | | | | | | |
| | | 7.68 (d, J = 1.7 Hz, 1H), 7.31 (t, | | | | | | |
| | | J = 8.8, 8.8 Hz, 1H), 4.12 (s, 3H), | | | | | | |
| | | 3.93 (s, 3H), 3.22 (d, J = 3.9 Hz, | | | | | | |
| | | 3H). | | | | | | |
| 718 | 2HCl | 1H NMR (400 MHz, DMSO) δ 9.54 | DMSO | >98 | R3 | | | |
| | | (d, J = 1.9 Hz, 1H), 9.41 (s, 1H), | | | | | | |
| | | 9.18 (d, J = 7.9 Hz, 1H), 9.01 (dd, | | | | | | |
| | | J = 5.5, 1.5 Hz, 1H), 8.12-8.01 | | | | | | |
| | | (m, 2H), 7.58-7.48 (m, 2H), 7.11 | | | | | | |
| | | (dd, J = 11.5, 2.5 Hz, 1H), 6.95 | | | | | | |
| | | (td, J = 8.4, 8.4, 2.5 Hz, 1H), 4.04 | | | | | | |
| | | (s, 3H), 3.85 (s, 3H), 3.20 (d, J = | | | | | | |
| | | 3.9 Hz, 3H). | | | | | | |
| 719 | 2HCl | 1H NMR (400 MHz, DMSO) δ 9.56 | DMSO | >98 | R3 | | | |
| | | (d, J = 1.9 Hz, 1H), 9.36-9.12 | | | | | | |
| | | (m, 2H), 9.00 (dd, J = 5.4, 1.5 Hz, | | | | | | |
| | | 1H), 8.13-8.02 (m, 2H), 7.60 (d, | | | | | | |
| | | J = 1.5 Hz, 1H), 7.39 (dd, J = 9.3, | | | | | | |
| | | 3.1 Hz, 1H), 7.32-7.24 (m, 1H), | | | | | | |
| | | 7.24-7.16 (m, 1H), 4.05 (s, 3H), | | | | | | |
| | | 3.82 (s, 3H), 3.21 (d, J = 4.1 Hz, | | | | | | |
| | | 3H). | | | | | | |
| 720 | HCl | 1H NMR (400 MHz, DMSO) δ 9.59 | DMSO | >98 | R3 | | | |
| | | (d, J = 1.7 Hz, 1H), 9.27 (d, J = | | | at 80° C. | | | |
| | | 8.1 Hz, 1H), 9.14 (s, 1H), 9.01 (d, | | | in | | | |
| | | J = 5.4 Hz, 1H), 8.11 (t, J = 6.8, | | | DME—EtOH—H₂O | | | |
| | | 6.8 Hz, 1H), 8.02 (d, J = 1.5 Hz, | | | | | | |
| | | 1H), 7.58-7.52 (m, 2H), 7.50 (d, | | | | | | |
| | | J = 1.6 Hz, 1H), 7.48-7.42 (m, | | | | | | |
| | | 1H), 4.04 (s, 3H), 3.19 (d, J = 4.2 | | | | | | |
| | | Hz, 3H). | | | | | | |
| 721 | 2HCl | 1H NMR (400 MHz, DMSO) δ 9.59 | DMSO | >98 | R3 | | | |
| | | (d, J = 1.9 Hz, 1H), 9.29 (d, J = | | | | | | |
| | | 8.1 Hz, 1H), 9.18 (s, 1H), 9.04 | | | | | | |
| | | (dd, J = 5.6, 1.5 Hz, 1H), 8.15 | | | | | | |
| | | (dd, J = 8.1, 5.5 Hz, 1H), 7.95- | | | | | | |
| | | 7.89 (m, 2H), 7.86-7.79 (m, 1H), | | | | | | |
| | | 7.76-7.67 (m, 1H), 7.59 (d, J = | | | | | | |
| | | 7.5 Hz, 1H), 7.40-7.34 (m, 1H), | | | | | | |
| | | 4.01 (s, 3H), 3.18 (d, J = 4.1 Hz, | | | | | | |
| | | 3H). | | | | | | |
| 722 | 2HCl | 1H NMR (400 MHz, DMSO) δ 9.61- | DMSO | >98 | R3 | | | |
| | | 9.55 (m, 1H), 9.41-9.20 (m, | | | | | | |
| | | 2H), 9.04 (dd, J = 5.6, 1.5 Hz, | | | | | | |
| | | 1H), 8.18-8.08 (m, 2H), 7.79- | | | | | | |
| | | 7.72 (m, 1H), 7.67-7.55 (m, 3H), | | | | | | |
| | | 7.52 (d, J = 1.6 Hz, 1H), 4.04 (s, | | | | | | |
| | | 3H), 3.20 (d, J = 4.0 Hz, 3H). | | | | | | |
| 723 | 2HCl | 1H NMR (400 MHz, DMSO) δ 9.59- | DMSO | >98 | R3 | | | |
| | | 9.54 (m, 1H), 9.36 (s, 1H), 9.22 | | | | | | |
| | | (d, J = 8.1 Hz, 1H), 9.00 (d, J = | | | | | | |
| | | 5.2 Hz, 1H), 8.36 (s, 1H), 8.29- | | | | | | |
| | | 8.21 (m, 2H), 8.08 (t, J = 6.8, 6.8 | | | | | | |
| | | Hz, 1H), 7.84-7.71 (m, 3H), 4.14 | | | | | | |
| | | (s, 3H), 3.23 (d, J = 4.3 Hz, 3H). | | | | | | |
| 724 | HCl | 1H NMR (400 MHz, DMSO) δ 9.58 | DMSO | >98 | R3 | | | |
| | | (d, J = 2.0 Hz, 1H), 9.15 (d, J = | | | | | | |
| | | 8.0 Hz, 2H), 8.94 (dd, J = 5.3, 1.6 | | | | | | |
| | | Hz, 1H), 8.29 (d, J = 1.7 Hz, 1H), | | | | | | |
| | | 8.03-7.95 (m, 2H), 7.95-7.89 | | | | | | |
| | | (m, 1H), 7.73-7.65 (m, 2H), 7.50- | | | | | | |
| | | 7.41 (m, 1H), 4.12 (s, 3H), 3.22 | | | | | | |
| | | (d, J = 4.3 Hz, 3H). | | | | | | |
| 725 | 2HCl | 1H NMR (400 MHz, DMSO) δ 9.63- | DMSO | >98 | R3 | | | |
| | | 9.59 (m, 1H), 9.00 (dt, J = 8.1, | | | | | | |
| | | 1.9, 1.9 Hz, 1H), 8.91 (s, 1H), | | | | | | |
| | | 8.83 (dd, J = 5.1, 1.6 Hz, 1H), | | | | | | |
| | | 8.28 (d, J = 1.7 Hz, 1H), 8.16- | | | | | | |
| | | 8.10 (m, 2H), 7.91 (d, J = 8.1 Hz, | | | | | | |
| | | 2H), 7.81 (dd, J = 8.0, 5.1 Hz, | | | | | | |
| | | 1H), 7.68 (d, J = 1.7 Hz, 1H), 4.11 | | | | | | |
| | | (s, 3H), 3.21 (d, J = 4.4 Hz, 3H). | | | | | | |
| 726 | 2HCl | 1H NMR (400 MHz, DMSO) δ 9.59- | DMSO | >98 | R3 | | | |
| | | 9.53 (m, 1H), 9.36 (s, 1H), 9.20 | | | | | | |
| | | (d, J = 7.8 Hz, 1H), 9.02-8.95 | | | | | | |
| | | (m, 1H), 8.36-8.30 (m, 1H), 8.10- | | | | | | |
| | | 8.02 (m, 3H), 7.70 (d, J = 1.7 | | | | | | |
| | | Hz, 1H), 7.59-7.51 (m, 2H), 4.12 | | | | | | |
| | | (s, 3H), 3.22 (d, J = 4.0 Hz, 3H). | | | | | | |
| 727 | HCl | 1H NMR (400 MHz, DMSO) δ 9.54- | DMSO | >98 | G7, | | | |
| | | 9.48 (m, 1H), 9.07 (d, J = 7.9 | | | R3 | | | |
| | | Hz, 1H), 8.93 (dd, J = 5.3, 1.5 Hz, | | | | | | |
| | | 1H), 7.97 (dd, J = 8.1, 5.3 Hz, | | | | | | |
| | | 1H), 7.72-7.67 (m, 1H), 7.63- | | | | | | |
| | | 7.45 (m, 3H), 7.42-7.32 (m, 1H), | | | | | | |
| | | 4.69 (brs, 4H), 4.07 (s, 3H), 2.56- | | | | | | |
| | | 2.44 (m, 2H). | | | | | | |
| 728 | HCl | 1H NMR (400 MHz, DMSO) δ 9.50 | DMSO | >98 | G7, | | | |
| | | (d, J = 2.0 Hz, 1H), 9.05 (d, J = | | | R3 | | | |
| | | 8.0 Hz, 1H), 8.92 (dd, J = 5.4, 1.6 | | | | | | |
| | | Hz, 1H), 7.95 (dd, J = 8.1, 5.3 Hz, | | | | | | |
| | | 1H), 7.88-7.76 (m, 1H), 7.64 (t, J = | | | | | | |
| | | 1.5, 1.5 Hz, 1H), 7.52 (s, 1H), | | | | | | |
| | | 7.48-7.39 (m, 1H), 7.31-7.22 | | | | | | |
| | | (m, 1H), 4.68 (brs, 4H), 4.06 (s, | | | | | | |
| | | 3H), 2.56-2.43 (m, 2H). | | | | | | |
| 729 | 2HCl | 1H NMR (400 MHz, DMSO) δ 9.61 | DMSO | >98 | G7, | | | |
| | | (dd, J = 2.2, 0.8 Hz, 1H), 9.02 (d, | | | R3 | | | |
| | | J = 8.1 Hz, 1H), 8.95 (dd, J = 5.1, | | | | | | |
| | | 1.6 Hz, 1H), 8.34-8.26 (m, 1H), | | | | | | |
| | | 8.25-8.20 (m, 1H), 8.20-8.15 | | | | | | |
| | | (m, 1H), 7.87 (dd, J = 8.1, 5.1 Hz, | | | | | | |
| | | 1H), 7.72-7.64 (m, 1H), 7.53- | | | | | | |
| | | 7.43 (m, 1H), 7.42-7.32 (m, 1H), | | | | | | |
| | | 4.86 (brs, 4H), 2.62-2.52 (m, | | | | | | |
| | | 2H). | | | | | | |
| 730 | 2HCl | 1H NMR (400 MHz, DMSO) δ 9.62- | DMSO | >98 | G7, | | | |
| | | 9.57 (m, 1H), 9.02-8.91 (m, | | | R3 | | | |
| | | 2H), 8.38-8.30 (m, 1H), 8.30- | | | | | | |
| | | 8.22 (m, 1H), 8.16 (d, J = 1.9 Hz, | | | | | | |
| | | 1H), 8.05-7.96 (m, 1H), 7.89- | | | | | | |
| | | 7.82 (m, 1H), 7.76-7.69 (m, 1H), | | | | | | |
| | | 7.66-7.55 (m, 1H), 4.86 (brs, | | | | | | |
| | | 4H), 2.63-2.53 (m, 2H). s | | | | | | |
| 731 | | 1H NMR (400 MHz, DMSO) δ 9.56 | DMSO | >98 | G7, | | | |
| | | (dd, J = 2.1, 0.9 Hz, 1H), 8.72- | | | R3 | | | |
| | | 8.63 (m, 2H), 7.99-7.90 (m, 1H), | | | | | | |
| | | 7.72-7.64 (m, 1H), 7.61 (d, J = | | | | | | |
| | | 1.8 Hz, 1H), 7.59-7.48 (m, 3H), | | | | | | |
| | | 4.65 (brs, 4H), 4.06 (s, 3H), 2.49- | | | | | | |
| | | 2.40 (m, 2H). | | | | | | |
| 732 | 2HCl | 1H NMR (400 MHz, DMSO) δ 9.49 | DMSO | >98 | G7, | | | |
| | | (d, J = 2.0 Hz, 1H), 9.13 (d, J = | | | R3 | | | |
| | | 7.9 Hz, 1H), 8.98 (dd, J = 5.5, 1.5 | | | | | | |
| | | Hz, 1H), 8.05 (dd, J = 8.1, 5.4 Hz, | | | | | | |
| | | 1H), 7.73-7.62 (m, 4H), 7.36- | | | | | | |
| | | 7.27 (m, 1H), 4.78 (brs, 4H), 4.12 | | | | | | |
| | | (s, 3H), 2.58-2.47 (m, 2H). | | | | | | |
| 733 | 2HCl | 1H NMR (400 MHz, DMSO) δ 9.50 | DMSO | >98 | G7, | | | |
| | | (d, J = 1.9 Hz, 1H), 9.14 (d, J = | | | R3 | | | |
| | | 8.0 Hz, 1H), 8.99 (dd, J = 5.5, 1.5 | | | | | | |
| | | Hz, 1H), 8.07 (dd, J = 8.1, 5.5 Hz, | | | | | | |
| | | 1H), 7.81-7.74 (m, 1H), 7.70 (t, J = | | | | | | |
| | | 1.5 Hz, 1H), 7.59 (t, J = 1.5 Hz, | | | | | | |
| | | 1H), 7.55-7.47 (m, 1H), 7.43- | | | | | | |
| | | 7.34 (m, 2H), 4.72 (brs, 4H), 4.08 | | | | | | |
| | | (s, 3H), 2.57-2.45 (m, 2H). | | | | | | |
| 734 | 2HCl | 1H NMR (400 MHz, DMSO) δ 9.49 | DMSO | >98 | G7, | | | |
| | | (d, J = 2.0 Hz, 1H), 9.09 (d, J = | | | R3 | | | |
| | | 7.9 Hz, 1H), 8.97 (dd, J = 5.4, 1.5 | | | | | | |
| | | Hz, 1H), 8.02 (dd, J = 8.1, 5.4 Hz, | | | | | | |
| | | 1H), 7.80-7.68 (m, 4H), 7.62- | | | | | | |
| | | 7.53 (m, 1H), 7.33-7.23 (m, 1H), | | | | | | |
| | | 4.82 (brs, 4H), 4.13 (s, 3H), 2.58- | | | | | | |
| | | 2.46 (m, 2H). | | | | | | |
| 735 | 2HCl | 1H NMR (400 MHz, DMSO) δ 9.48 | DMSO | >98 | G7, | | | |
| | | (d, J = 2.0 Hz, 1H), 9.09 (d, J = | | | R3 | | | |
| | | 7.9 Hz, 1H), 8.97 (dd, J = 5.4, 1.5 | | | | | | |
| | | Hz, 1H), 8.03 (dd, J = 8.0, 5.4 Hz, | | | | | | |
| | | 1H), 7.96-7.87 (m, 2H), 7.68 (s, | | | | | | |
| | | 2H), 7.41-7.32 (m, 2H), 4.78 | | | | | | |
| | | (brs, 4H), 4.12 (s, 3H), 2.58- | | | | | | |
| | | 2.47 (m, 2H). | | | | | | |
| 736 | HCl | 1H NMR (400 MHz, DMSO) δ | DMSO | >98 | G7, | | | |
| | | 9.57-9.52 (m, 1H), 9.01 (d, J = 7.9 | | | R3 | | | |
| | | Hz, 1H), 8.88 (dd, J = 5.3, 1.6 Hz, | | | | | | |
| | | 1H), 8.05-8.00 (m, 1H), 7.92- | | | | | | |
| | | 7.85 (m, 3H), 7.72 (d, J = 1.7 Hz, | | | | | | |
| | | 1H), 7.69-7.62 (m, 1H), 7.60 (d, | | | | | | |
| | | J = 1.7 Hz, 1H), 4.72 (brs, 4H), | | | | | | |
| | | 4.08 (s, 3H), 2.58-2.48 (m, 2H). | | | | | | |
| 737 | | 1H NMR (400 MHz, DMSO) δ 9.57 | DMSO | >98 | G7, | | | |
| | | (dd, J = 2.2, 0.9 Hz, 1H), 8.71 (dt, | | | R3 | | | |
| | | J = 8.0, 1.9 Hz, 1H), 8.67 (dd, J = | | | | | | |
| | | 4.8, 1.7 Hz, 1H), 7.75 (s, 1H), 7.63- | | | | | | |
| | | 7.58 (m, 1H), 7.58-7.50 (m, | | | | | | |
| | | 3H), 7.50-7.41 (m, 3H), 7.36 (d, | | | | | | |
| | | J = 1.7 Hz, 1H), 4.61 (brs, 4H), | | | | | | |
| | | 4.01 (s, 3H), 2.49-.41 (m, 2H). | | | | | | |
| 738 | | 1H NMR (400 MHz, DMSO) δ 9.56 | DMSO | >98 | G7, | | | |
| | | (dd, J = 2.1, 0.9 Hz, 1H), 8.72- | | | R3 | | | |
| | | 8.64 (m, 2H), 8.33 (t, J = 1.5 Hz, | | | | | | |
| | | 1H), 8.15 (ddd, J = 8.0, 2.0, 1.1 | | | | | | |
| | | Hz, 1H), 7.89-7.83 (m, 1H), 7.73- | | | | | | |
| | | 7.64 (m, 2H), 7.58 (d, J = 1.8 | | | | | | |
| | | Hz, 1H), 7.52 (ddd, J = 8.0, 4.8, | | | | | | |
| | | 0.9 Hz, 1H), 4.65 (brs, 4H), 4.07 | | | | | | |
| | | (s, 3H), 2.49-2.40 (m, 2H). | | | | | | |
| 739 | | 1H NMR (400 MHz, DMSO) δ 9.57 | DMSO | >98 | G7, | | | |
| | | (dd, J = 2.0, 0.9 Hz, 1H), 8.74- | | | R3 | | | |
| | | 8.63 (m, 2H), 8.09-8.02 (m, 2H), | | | | | | |
| | | 8.00-7.93 (m, 2H), 7.73 (d, J = | | | | | | |
| | | 1.8 Hz, 1H), 7.59 (d, J = 1.7 Hz, | | | | | | |
| | | 1H), 7.53 (ddd, J = 8.0, 4.8, 0.9 | | | | | | |
| | | Hz, 1H), 4.67 (brs, 4H), 4.07 (s, | | | | | | |
| | | 3H), 2.50-2.42 (m, 2H). | | | | | | |
| 740 | 2HCl | 1H NMR (400 MHz, DMSO) δ | DMSO | >98 | G5, | | | |
| | | 9.55-9.49 (m, 1H), 9.20 (d, J = 7.7 | | | R3 | | | |
| | | Hz, 1H), 9.03-8.95 (m, 1H), 8.14- | | | | | | |
| | | 8.03 (m, 1H), 7.99-7.90 (m, | | | | | | |
| | | 2H), 7.71-7.63 (m, 2H), 7.41- | | | | | | |
| | | 7.30 (m, 2H), 4.93 (brs, 2H), 4.69- | | | | | | |
| | | 4.41 (m, 3H), 4.11 (s, 3H), 3.35 | | | | | | |
| | | (s, 3H). | | | | | | |
| 741 | 2HCl | 1H NMR (400 MHz, DMSO) δ | DMSO | >98 | G5, R3 | | | |
| | | 9.56-9.50 (m, 1H), 9.24 (d, J = 8.0 | | | | | | |
| | | Hz, 1H), 9.01 (dd, J = 5.2, 1.8 Hz, | | | | | | |
| | | 1H), 8.17-8.08 (m, 1H), 7.90- | | | | | | |
| | | 7.79 (m, 1H), 7.65 (s, 1H), 7.54 (s, | | | | | | |
| | | 1H), 7.51-7.41 (m, 1H), 7.28 (td, | | | | | | |
| | | J = 8.5, 2.5 Hz, 1H), 4.87 (brs, | | | | | | |
| | | 2H), 4.60-4.39 (m, 3H), 4.06 (s, | | | | | | |
| | | 3H), 3.33 (s, 3H). | | | | | | |
| 742 | HCl | 1H NMR (400 MHz, DMSO) δ 10.28 | DMSO | >98 | R3 | | | |
| | | (s, 1H), 9.68-9.62 (m, 1H), 9.03 | | | | | | |
| | | (d, J = 8.1 Hz, 1H), 9.00-8.91 | | | | | | |
| | | (m, 2H), 8.39 (dd, J = 8.8, 2.0 Hz, | | | | | | |
| | | 1H), 8.21 (d, J = 8.7 Hz, 1H), 7.87 | | | | | | |
| | | (dd, J = 8.1, 5.0 Hz, 1H), 7.83- | | | | | | |
| | | 7.77 (m, 1H), 7.76 (t, J = 2.1 Hz, | | | | | | |
| | | 1H), 7.69-7.24 (m, 3H), 3.31 (d, | | | | | | |
| | | J = 4.4 Hz, 3H). | | | | | | |
| 743 | HCl | 1H NMR (400 MHz, DMSO) δ 9.92- | DMSO | >98 | R3 | | | |
| | | 9.60 (m, 2H), 8.99 d, J = 7.8 | | | | | | |
| | | Hz, 1H), 8.94-8.89 (m, 1H), 8.52 | | | | | | |
| | | (d, J = 8.6 Hz, 1H), 8.32 (s, 1H), | | | | | | |
| | | 8.09 (dd, J = 8.4, 1.5 Hz, 1H), | | | | | | |
| | | 7.83 (dd, J = 8.1, 5.0 Hz, 1H), | | | | | | |
| | | 7.78-7.72 (m, 1H), 7.69-7.61 | | | | | | |
| | | (m, 2H), 7.61-7.21 (m, 2H), 3.28 | | | | | | |
| | | (d, J = 4.5 Hz, 3H). | | | | | | |
| 744 | 2HCl | 1H NMR (400 MHz, DMSO) δ 9.57 | DMSO | >98 | R3 | | | |
| | | (d, J = 2.0 Hz, 1H), 9.42 (s, 1H), | | | | | | |
| | | 9.20 (d, J = 8.2 Hz, 1H), 8.99 (dd, | | | | | | |
| | | J = 5.4, 1.5 Hz, 1H), 8.38 (s, 1H), | | | | | | |
| | | 8.06 (dd, J = 8.0, 5.4 Hz, 1H), | | | | | | |
| | | 7.83 (ddd, J = 7.8, 1.7, 0.9 Hz, | | | | | | |
| | | 1H), 7.78 (t, J = 2.1, 2.1 Hz, 1H), | | | | | | |
| | | 7.72 (d, J = 1.7 Hz, 1H), 7.68- | | | | | | |
| | | 7.24 (m, 3H), 4.13 (s, 3H), 3.23 | | | | | | |
| | | (d, J = 4.2 Hz, 3H). | | | | | | |
| 745 | HCl | 1H NMR (400 MHz, DMSO) δ 9.88 | DMSO | >98 | R3 | | | |
| | | (s, 1H), 9.62 (dd, J = 2.2, 0.9 Hz, | | | | | | |
| | | 1H), 8.96 (d, J = 7.8 Hz, 1H), 8.92 | | | | | | |
| | | (dd, J = 5.0, 1.6 Hz, 1H), 8.39 (s, | | | | | | |
| | | 1H), 8.02 (s, 1H), 7.81 (dd, J = | | | | | | |
| | | 7.8, 5.1 Hz, 1H), 7.64-7.20 (m, | | | | | | |
| | | 5H), 3.25 (d, J = 4.5 Hz, 3H), 2.46 | | | | | | |
| | | (s, 3H). | | | | | | |
| 746 | HCl | 1H NMR (400 MHz, DMSO) δ 10.53 | DMSO | >98 | R3 | | | |
| | | (s, 1H), 9.86 (d, J = 1.5 Hz, 1H), | | | | | | |
| | | 9.07 (d, J = 2.5 Hz, 1H), 9.02 (dd, | | | | | | |
| | | J = 2.4, 1.4 Hz, 1H), 8.66 (d, J = | | | | | | |
| | | 8.7 Hz, 1H), 8.54 (d, J = 1.9 Hz, | | | | | | |
| | | 1H), 8.20 (dd, J = 8.6, 1.9 Hz, | | | | | | |
| | | 1H), 7.77-7.71 (m, 1H), 7.71- | | | | | | |
| | | 7.63 (m, 2H), 7.61-7.19 (m, 2H), | | | | | | |
| | | 3.38 (d, J = 4.5 Hz, 3H). | | | | | | |
| 747 | HCl | 1H NMR (400 MHz, DMSO) δ 10.28 | DMSO | >98 | R3 | | | |
| | | (s, 1H), 9.69 (dd, J = 2.4, 0.9 Hz, | | | | | | |
| | | 1H), 9.09 (dt, J = 8.2, 1.9 Hz, 1H), | | | | | | |
| | | 8.97 (dd, J = 5.0, 1.6 Hz, 1H), | | | | | | |
| | | 8.68 (d, J = 1.9 Hz, 1H), 8.29 (d, | | | | | | |
| | | J = 8.7 Hz, 1H), 8.14 (dd, J = 8.7, | | | | | | |
| | | 1.8 Hz, 1H), 7.89 (dd, J = 8.1, 5.0 | | | | | | |
| | | Hz, 1H), 7.64 (dd, J = 7.6, 1.8 Hz, | | | | | | |
| | | 1H), 7.61-7.52 (m, 1H), 7.48- | | | | | | |
| | | 7.02 (m, 3H), 3.29 (d, J = 4.4 Hz, | | | | | | |
| | | 3H). | | | | | | |
| 748 | 2HCl | 1H NMR (400 MHz, DMSO) δ 9.60- | DMSO | >98 | R3 | | | |
| | | 9.55 (m, 1H), 9.31-9.13 (m, | | | | | | |
| | | 2H), 9.03-8.96 (m, 1H), 8.12- | | | | | | |
| | | 8.01 (m, 2H), 7.65 (dd, J = 7.6, | | | | | | |
| | | 1.7 Hz, 1H), 7.59-.51 (m, 2H), | | | | | | |
| | | 7.48-7.01 (m, 3H), 4.03 (s, 3H), | | | | | | |
| | | 3.21 (d, J = 4.2 Hz, 3H). | | | | | | |
| 749 | HCl | 1H NMR (400 MHz, DMSO) δ 10.21 | DMSO | >98 | R3 | | | |
| | | (s, 1H), 9.65 (d, J = 2.2 Hz, 1H), | | | | | | |
| | | 9.05 (d, J = 6.9 Hz, 1H), 8.96 (dd, | | | | | | |
| | | J = 5.1, 1.5 Hz, 1H), 8.59 (d, J = | | | | | | |
| | | 8.5 Hz, 1H), 8.26 (s, 1H), 7.92- | | | | | | |
| | | 7.80 (m, 2H), 7.64-7.56 (m, 2H), | | | | | | |
| | | 7.50-7.04 (m, 3H), 3.31 (d, J = | | | | | | |
| | | 4.4 Hz, 3H). | | | | | | |
| 750 | HCl | 1H NMR (400 MHz, DMSO) δ 9.60- | DMSO | >98 | R3 | | | |
| | | 9.54 (m, 1H), 9.26-9.04 (m, | | | | | | |
| | | 2H), 8.92 (dd, J = 5.3, 1.5 Hz, | | | | | | |
| | | 1H), 8.25 (s, 1H), 8.04-7.88 (m, | | | | | | |
| | | 3H), 7.66 (d, J = 1.7 Hz, 1H), 7.58- | | | | | | |
| | | 7.12 (m, 3H), 4.11 (s, 3H), 3.22 | | | | | | |
| | | (d, J = 4.3 Hz, 3H). | | | | | | |
| 751 | HCl | 1H NMR (400 MHz, DMSO) δ 10.04 | DMSO | >98 | R3 | | | |
| | | (s, 1H), 9.65 (dd, J = 2.3, 0.8 Hz, | | | | | | |
| | | 1H), 9.02 (d, J = 7.8 Hz, 1H), 8.92 | | | | | | |
| | | (dd, J = 5.0, 1.6 Hz, 1H), 8.56 (d, | | | | | | |
| | | J = 8.6 Hz, 1H), 8.43 (s, 1H), 8.05 | | | | | | |
| | | (dd, J = 8.6, 1.9 Hz, 1H), 7.96- | | | | | | |
| | | 7.88 (m, 2H), 7.82 (dd, J = 8.1, | | | | | | |
| | | 5.0 Hz, 1H), 7.60-7.15 (m, 3H), | | | | | | |
| | | 3.28 (d, J = 4.4 Hz, 3H). | | | | | | |
| 752 | 2HCl | 1H NMR (400 MHz, DMSO) δ 10.04 | DMSO | >98 | R3 | | | |
| | | (s, 1H), 9.67-9.61 (m, 1H), 9.01 | | | | | | |
| | | (d, J = 7.8 Hz, 1H), 8.96 (dd, J = | | | | | | |
| | | 5.0, 1.5 Hz, 1H), 8.36 (s, 1H), | | | | | | |
| | | 8.10 (s, 1H), 7.92-7.83 (m, 1H), | | | | | | |
| | | 7.62-7.55 (m, 1H), 7.45-7.00 | | | | | | |
| | | (m, 4H), 3.26 (d, J = 4.5 Hz, 3H), | | | | | | |
| | | 2.30 (s, 3H). | | | | | | |
| 753 | HCl | 1H NMR (400 MHz, DMSO) δ 9.75 | DMSO | >98 | R3 | | | |
| | | (s, 1H), 9.61 (d, J = 2.1 Hz, 1H), | | | | | | |
| | | 8.98-8.87 (m, 2H), 8.32 (s, 1H), | | | | | | |
| | | 7.97 (s, 1H), 7.80 (dd, J = 8.0, 5.0 | | | | | | |
| | | Hz, 1H), 7.60-7.15 (m, 5H), 3.24 | | | | | | |
| | | (d, J = 4.4 Hz, 3H), 2.45 (s, 3H). | | | | | | |
| 754 | HCl | 1H NMR (400 MHz, DMSO) δ 10.69 | DMSO | >98 | R3 | | | |
| | | (s, 1H), 9.84 (d, J = 1.4 Hz, 1H), | | | | | | |
| | | 9.07 (d, J = 2.4 Hz, 1H), 9.01 (dd, | | | | | | |
| | | J = 2.4, 1.5 Hz, 1H), 8.70 (d, J = | | | | | | |
| | | 8.7 Hz, 1H), 8.51 (d, J = 1.8 Hz, | | | | | | |
| | | 1H), 8.15 (dd, J = 8.6, 1.8 Hz, | | | | | | |
| | | 1H), 7.95-7.86 (m, 2H), 7.60- | | | | | | |
| | | 7.17 (m, 3H), 3.38 (d, J = 4.5 Hz, | | | | | | |
| | | 3H). | | | | | | |
| 755 | HCl | 1H NMR (400 MHz, DMSO) δ 10.54 | DMSO | >98 | R3 | | | |
| | | (s, 1H), 9.86 (d, J = 1.4 Hz, 1H), | | | | | | |
| | | 9.07 (dd, J = 2.5, 0.9 Hz, 1H), | | | | | | |
| | | 9.04-8.98 (m, 1H), 8.68-8.56 | | | | | | |
| | | (m, 1H), 8.37 (s, 1H), 7.92 (dd, J = | | | | | | |
| | | 8.6, 1.7 Hz, 1H), 7.64-7.57 (m, | | | | | | |
| | | 2H), 7.49-7.09 (m, 3H), 3.39 (d, | | | | | | |
| | | J = 4.5 Hz, 3H). | | | | | | |
| 756 | 2HCl | 1H NMR (400 MHz, DMSO) δ 9.57- | DMSO | >98 | G5, R3 | | | |
| | | 9.50 (m, 1H), 9.25 (d, J = 8.1 | | | | | | |
| | | Hz, 1H), 9.05-8.98 (m, 1H), 8.17- | | | | | | |
| | | 8.08 (m, 1H), 7.73-7.67 (m, | | | | | | |
| | | 1H), 7.65-7.47 (m, 3H), 7.43- | | | | | | |
| | | 7.32 (m, 1H), 4.89 (brs, 2H), 4.58- | | | | | | |
| | | 4.39 (m, 3H), 4.07 (s, 3H), 3.33 | | | | | | |
| | | (s, 3H). | | | | | | |
| 757 | 2HCl | 1H NMR (400 MHz, DMSO) δ | DMSO | >98 | G5, R3 | | | |
| | | 9.56-9.51 (m, 1H), 9.25 (d, J = 8.3 | | | | | | |
| | | Hz, 1H), 9.04-8.99 (m, 1H), 8.12 | | | | | | |
| | | (dd, J = 7.9, 5.8 Hz, 1H), 7.75- | | | | | | |
| | | 7.66 (m, 2H), 7.59-7.53 (m, 1H), | | | | | | |
| | | 7.50-7.40 (m, 1H), 7.39-7.30 | | | | | | |
| | | (m, 1H), 4.88 (brs, 2H), 4.58- | | | | | | |
| | | 4.41 (m, 3H), 4.07 (s, 3H), 3.33 | | | | | | |
| | | (s, 3H). | | | | | | |
| 758 | HCl | 1H NMR (400 MHz, DMSO) δ 9.60 | DMSO | >98 | R3 | | | |
| | | (dd, J = 2.1, 0.7 Hz, 1H), 9.14 (d, | | | | | | |
| | | J = 8.1 Hz, 1H), 9.04-8.89 (m, | | | | | | |
| | | 2H), 8.03 (d, J = 1.6 Hz, 1H), 7.97 | | | | | | |
| | | (dd, J = 8.0, 5.3 Hz, 1H), 7.60- | | | | | | |
| | | 7.51 (m, 2H), 7.47-7.36 (m, 2H), | | | | | | |
| | | 7.16 (t, J = 73.7 Hz, 1H), 4.03 (s, | | | | | | |
| | | 3H), 3.20 (d, J = 4.3 Hz, 3H). | | | | | | |
| 759 | HCl | 1H NMR (400 MHz, DMSO) δ 9.91 | DMSO | >98 | R3 | | | |
| | | (s, 1H), 9.63 (dd, J = 2.0, 0.9 Hz, | | | | | | |
| | | 1H), 8.99 (d, J = 8.1 Hz, 1H), 8.92 | | | | | | |
| | | (dd, J = 5.0, 1.6 Hz, 1H), 8.57 (d, | | | | | | |
| | | J = 1.8 Hz, 1H), 8.18-8.11 (m, | | | | | | |
| | | 1H), 8.11-8.03 (m, 1H), 7.84 | | | | | | |
| | | (dd, J = 8.2, 5.0 Hz, 1H), 7.68 (dd, | | | | | | |
| | | J = 8.3, 6.5 Hz, 1H), 7.51-7.09 | | | | | | |
| | | (m, 3H), 3.27 (d, J = 4.5 Hz, 3H). | | | | | | |
| 760 | HCl | 1H NMR (400 MHz, DMSO) δ 9.57 | DMSO | >98 | R3 | | | |
| | | (d, J = 1.8 Hz, 1H), 9.40-9.17 | | | | | | |
| | | (m, 2H), 9.01 (dd, J = 5.5, 1.5 Hz, | | | | | | |
| | | 1H), 8.13-8.04 (m, 2H), 7.76- | | | | | | |
| | | 7.67 (m, 1H), 7.56-7.12 (m, 4H), | | | | | | |
| | | 4.03 (s, 3H), 3.20 (d, J = 4.3 Hz, | | | | | | |
| | | 3H). | | | | | | |
| 761 | HCl | 1H NMR (400 MHz, DMSO) δ 9.90 | DMSO | >98 | R3 | | | |
| | | (s, 1H), 9.63 (dd, J = 1.9, 0.8 Hz, | | | | | | |
| | | 1H), 8.98 (d, J = 8.1 Hz, 1H), 8.91 | | | | | | |
| | | (dd, J = 5.1, 1.6 Hz, 1H), 8.52 (d, | | | | | | |
| | | J = 8.6 Hz, 1H), 8.14 (s, 1H), 7.87- | | | | | | |
| | | 7.76 (m, 2H), 7.67 (dd, J = 8.6, | | | | | | |
| | | 6.5 Hz, 1H), 7.55-7.13 (m, 3H), | | | | | | |
| | | 3.28 (d, J = 4.4 Hz, 3H). | | | | | | |
| 762 | HCl | 1H NMR (400 MHz, DMSO) δ 10.13 | DMSO | >98 | R3 | | | |
| | | (s, 1H), 9.66 (d, J = 2.1 Hz, 1H), | | | | | | |
| | | 9.06 (d, J = 8.1 Hz, 1H), 8.97 (dd, | | | | | | |
| | | J = 5.2, 1.6 Hz, 1H), 8.38 (s, 1H), | | | | | | |
| | | 8.14 (s, 1H), 7.89 (dd, J = 8.1, 5.0 | | | | | | |
| | | Hz, 1H), 7.54-7.11 (m, 4H), 3.26 | | | | | | |
| | | (d, J = 4.5 Hz, 3H), 2.29 (s, 3H). | | | | | | |
| 763 | HCl | 1H NMR (400 MHz, DMSO) δ 10.55 | DMSO | >98 | R3 | | | |
| | | (s, 1H), 9.85 (d, J = 1.4 Hz, 1H), | | | | | | |
| | | 9.06 (d, J = 2.4 Hz, 1H), 9.00 (dd, | | | | | | |
| | | J = 2.4, 1.4 Hz, 1H), 8.68-8.60 | | | | | | |
| | | (m, 1H), 8.34 (d, J = 2.0 Hz, 1H), | | | | | | |
| | | 7.89 (dd, J = 8.6, 1.7 Hz, 1H), | | | | | | |
| | | 7.68 (dd, J = 8.6, 6.5 Hz, 1H), | | | | | | |
| | | 7.58-7.13 (m, 3H), 3.38 (d, J = | | | | | | |
| | | 5.2 Hz, 3H). | | | | | | |
| 764 | HCl | 1H NMR (400 MHz, DMSO) δ 10.18 | DMSO | >98 | R3 | | | |
| | | (s, 1H), 9.67 (dd, J = 2.2, 0.8 Hz, | | | | | | |
| | | 1H), 9.09 (d, J = 8.1 Hz, 1H), 8.97 | | | | | | |
| | | (dd, J = 5.1, 1.6 Hz, 1H), 8.69 (d, | | | | | | |
| | | J = 1.9 Hz, 1H), 8.25 (d, J = 8.7 | | | | | | |
| | | Hz, 1H), 8.15 (dd, J = 8.6, 1.8 Hz, | | | | | | |
| | | 1H), 7.91 (dd, J = 7.9, 5.0 Hz, | | | | | | |
| | | 1H), 7.56 (dd, J = 9.1, 2.7 Hz, | | | | | | |
| | | 1H), 7.49-7.38 (m, 2H), 7.16 (t, | | | | | | |
| | | J = 73.5 Hz, 1H), 3.30 (d, J = 4.4 | | | | | | |
| | | Hz, 3H). | | | | | | |
| 765 | HCl | 1H NMR (400 MHz, DMSO) δ 9.56 | DMSO | >98 | R3 | | | |
| | | (d, J = 2.0 Hz, 1H), 9.39 (s, 1H), | | | | | | |
| | | 9.20 (d, J = 8.0 Hz, 1H), 8.99 (dd, | | | | | | |
| | | J = 5.4, 1.5 Hz, 1H), 8.33 (d, J = | | | | | | |
| | | 1.7 Hz, 1H), 8.06 (dd, J = 8.1, 5.4 | | | | | | |
| | | Hz, 1H), 7.70 (d, J = 1.7 Hz, 1H), | | | | | | |
| | | 7.44-7.36 (m, 2H), 6.99-6.89 | | | | | | |
| | | (m, 1H), 4.28-4.22 (m, 2H), 4.13 | | | | | | |
| | | (s, 3H), 3.74-3.68 (m, 2H), 3.34 | | | | | | |
| | | (s, 3H), 3.22 (d, J = 4.1 Hz, 3H). | | | | | | |

TABLE 27

| | Starting | Starting | | | | | Salt |
|---|---|---|---|---|---|---|---|
| Number | Material 1 | Material 2 | | Product | | | type |
| 766 | 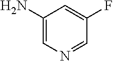 | 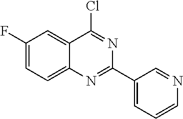 [SMILES: Fc1ccc2nc(-c3cccnc3)nc(Cl)c2c1] | | 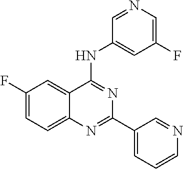 [SMILES: Fc1cncc(Nc2nc(-c3cccnc3)nc3ccc(F)cc23)c1] | | | HCl |
| 767 | 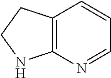 | 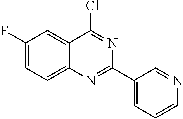 [SMILES: Fc1ccc2nc(-c3cccnc3)nc(Cl)c2c1] | | 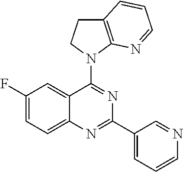 [SMILES: Fc1ccc2nc(-c3cccnc3)nc(N3CCc4cccnc43)c2c1] | | | 2HCl |
| 768 | 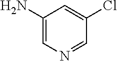 | 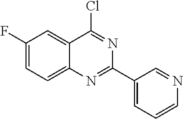 [SMILES: Fc1ccc2nc(-c3cccnc3)nc(Cl)c2c1] | | 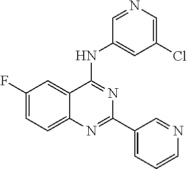 [SMILES: Fc1ccc2nc(-c3cccnc3)nc(Nc3cncc(Cl)c3)c2c1] | | | HCl |
| 769 | 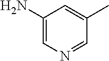 | 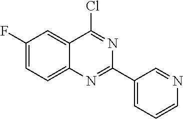 [SMILES: Fc1ccc2nc(-c3cccnc3)nc(Cl)c2c1] | | 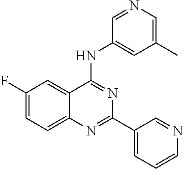 [SMILES: Cc1cncc(Nc2nc(-c3cccnc3)nc3ccc(F)cc23)c1] | | | 2HCl |
| 770 | 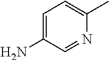 | 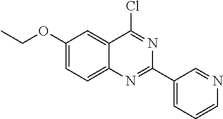 [SMILES: CCOc1ccc2nc(-c3cccnc3)nc(Cl)c2c1] | | 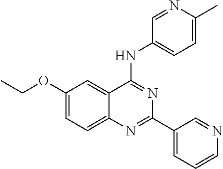 [SMILES: CCOc1ccc2nc(-c3cccnc3)nc(Nc3ccc(C)nc3)c2c1] | | | 3HCl |
| 771 | 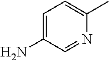 | 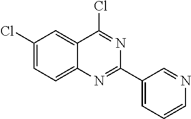 [SMILES: Clc1ccc2nc(-c3cccnc3)nc(Cl)c2c1] | | 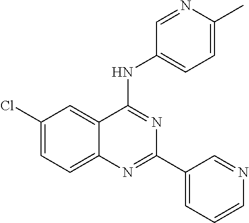 [SMILES: Cc1ccc(Nc2nc(-c3cccnc3)nc3ccc(Cl)cc23)cn1] | | | 3HCl |
| 772 | 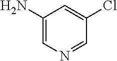 | 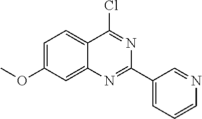 [SMILES: COc1ccc2c(Cl)nc(-c3cccnc3)nc2c1] | | 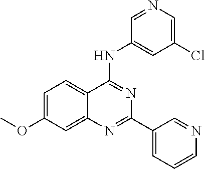 [SMILES: COc1ccc2c(Nc3cncc(Cl)c3)nc(-c3cccnc3)nc2c1] | | | 2HCl |
| 773 | 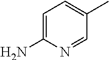 | 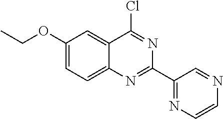 [SMILES: CCOc1ccc2nc(-c3cnccn3)nc(Cl)c2c1] | | 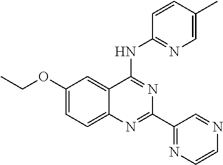 [SMILES: CCOc1ccc2nc(-c3cnccn3)nc(Nc3ccc(C)cn3)c2c1] | | | HCl |
| 774 | 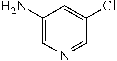 | 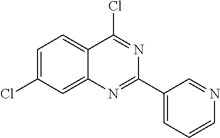 [SMILES: Clc1ccc2c(Cl)nc(-c3cccnc3)nc2c1] | | 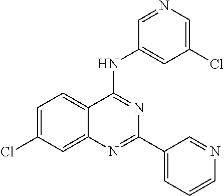 [SMILES: Clc1cncc(Nc2nc(-c3cccnc3)nc3cc(Cl)ccc23)c1] | | | 2HCl |
| 775 | 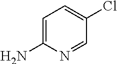 | 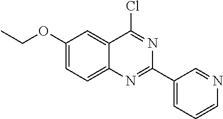 [SMILES: CCOc1ccc2nc(-c3cccnc3)nc(Cl)c2c1] | | 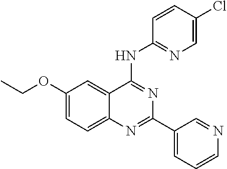 [SMILES: CCOc1ccc2nc(-c3cccnc3)nc(Nc3ccc(Cl)cn3)c2c1] | | | 2HCl |
| 776 | 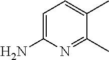 | 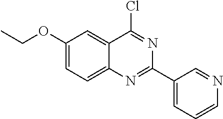 [SMILES: CCOc1ccc2nc(-c3cccnc3)nc(Cl)c2c1] | | 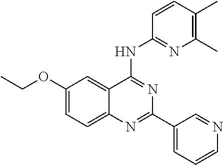 [SMILES: CCOc1ccc2nc(-c3cccnc3)nc(Nc3ccc(C)c(C)n3)c2c1] | | | 2HCl |
| 777 | 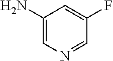 | 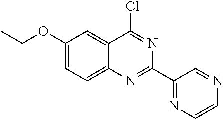 [SMILES: CCOc1ccc2nc(-c3cnccn3)nc(Cl)c2c1] | | 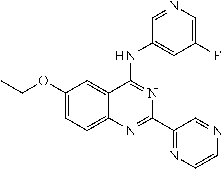 [SMILES: CCOc1ccc2nc(-c3cnccn3)nc(Nc3cncc(F)c3)c2c1] | | | |
| 778 | 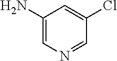 | 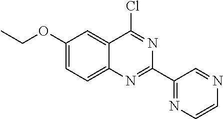 [SMILES: CCOc1ccc2nc(-c3cnccn3)nc(Cl)c2c1] | | 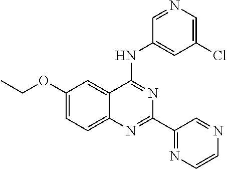 [SMILES: CCOc1ccc2nc(-c3cnccn3)nc(Nc3cncc(Cl)c3)c2c1] | | | |
| 779 | 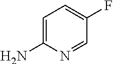 | 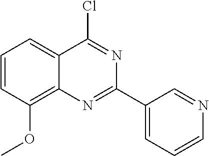 [SMILES: COc1cccc2c(Cl)nc(-c3cccnc3)nc12] | | 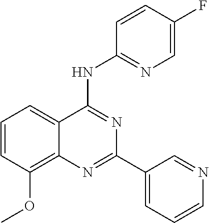 [SMILES: COc1cccc2c(Nc3ccc(F)cn3)nc(-c3cccnc3)nc12] | | | HCl |
| 780 | 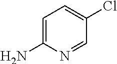 | 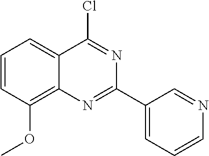 [SMILES: COc1cccc2c(Cl)nc(-c3cccnc3)nc12] | | 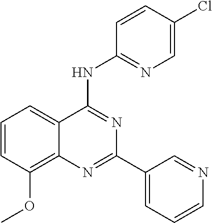 [SMILES: COc1cccc2c(Nc3ccc(Cl)cn3)nc(-c3cccnc3)nc12] | | | HCl |
| 781 | 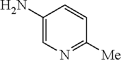 | 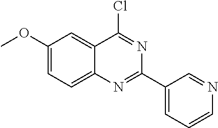 [SMILES: COc1ccc2nc(-c3cccnc3)nc(Cl)c2c1] | | 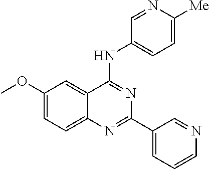 [SMILES: COc1ccc2nc(-c3cccnc3)nc(Nc3ccc(C)nc3)c2c1] | | | 2HCl |
| 782 | 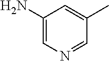 | 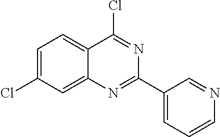 [SMILES: Clc1ccc2c(Cl)nc(-c3cccnc3)nc2c1] | | 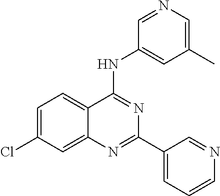 [SMILES: Cc1cncc(Nc2nc(-c3cccnc3)nc3cc(Cl)ccc23)c1] | | | |
| 783 | 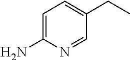 | 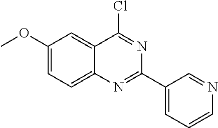 [SMILES: COc1ccc2nc(-c3cccnc3)nc(Cl)c2c1] | | 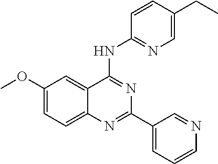 [SMILES: CCc1ccc(Nc2nc(-c3cccnc3)nc3ccc(OC)cc23)nc1] | | | 2HCl |
| 784 | 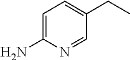 | 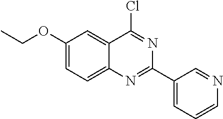 [SMILES: CCOc1ccc2nc(-c3cccnc3)nc(Cl)c2c1] | | 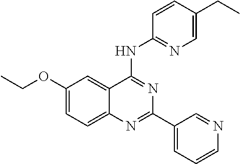 [SMILES: CCOc1ccc2nc(-c3cccnc3)nc(Nc3ccc(CC)cn3)c2c1] | | | 2HCl |
| 785 | 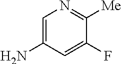 | 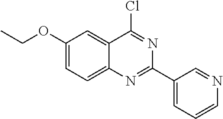 [SMILES: CCOc1ccc2nc(-c3cccnc3)nc(Cl)c2c1] | | 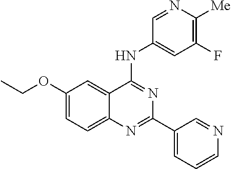 [SMILES: CCOc1ccc2nc(-c3cccnc3)nc(Nc3cnc(C)c(F)c3)c2c1] | | | 2HCl |
| 786 | 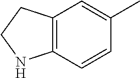 | 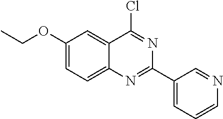 [SMILES: CCOc1ccc2nc(-c3cccnc3)nc(Cl)c2c1] | | 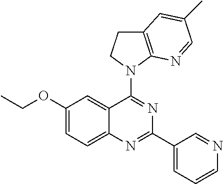 [SMILES: CCOc1ccc2nc(-c3cccnc3)nc(N3CCc4cc(C)cnc43)c2c1] | | | 2HCl |
| 787 | 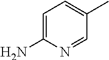 | 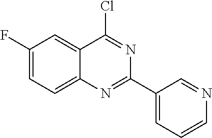 [SMILES: Fc1ccc2nc(-c3cccnc3)nc(Cl)c2c1] | | 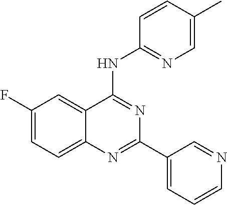 [SMILES: Cc1ccc(Nc2nc(-c3cccnc3)nc3ccc(F)cc23)nc1] | | | 2HCl |
| | | ¹H NMR | Purity | Method | | Retention | LCMS |
| Number | ¹H NMR | Solvent | percent | of Coupling | LCMS | Time | Method |
| 766 | 1H NMR (400 MHz, DMSO) δ 10.44 | DMSO | >98 | D, F3, H1 | | | |
| | (s, 1H), 9.50 (d, J = 1.9 Hz, 1H), | | | | | | |
| | 9.05-8.96 (m, 1H), 8.90 (dd, J = | | | | | | |
| | 5.3, 1.6 Hz, 1H), 8.79-8.73 (m, | | | | | | |
| | 1H), 8.56 (dd, J = 9.9, 2.8 Hz, 1H), | | | | | | |
| | 8.49 (ddd, J = 8.8, 7.3, 2.8 Hz, 1H), | | | | | | |
| | 8.05 (dd, J = 9.2, 5.4 Hz, 1H), 7.96- | | | | | | |
| | 7.87 (m, 2H), 7.34 (dd, J = 8.8, | | | | | | |
| | 3.2 Hz, 1H). | | | | | | |
| 767 | 1H NMR (400 MHz, DMSO) δ 9.72- | DMSO | >98 | D, F3, H1 | | | |
| | 9.68 (m, 1H), 9.40-9.30 (m, 1H), | | | | | | |
| | 9.05-8.99 (m, 1H), 8.19-8.09 | | | | | | |
| | (m, 2H), 7.99-7.88 (m, 3H), 7.82- | | | | | | |
| | 7.76 (m, 1H), 7.06-7.01 (m, | | | | | | |
| | 1H), 4.54 (t, J = 7.9 Hz, 2H), 3.30 | | | | | | |
| | (t, J = 7.9 Hz, 2H). | | | | | | |
| 768 | 1H NMR (400 MHz, DMSO) δ 10.61 | DMSO | >98 | D, F3, H1 | | | |
| | (s, 1H), 9.49-9.44 (m, 1H), 9.15 | | | | | | |
| | (d, J = 2.1 Hz, 1H), 9.06-9.00 (m, | | | | | | |
| | 1H), 8.94 (dd, J = 5.4, 1.5 Hz, 1H), | | | | | | |
| | 8.64 (dd, J = 9.9, 2.8 Hz, 1H), 8.59- | | | | | | |
| | 8.55 (m, 1H), 8.46 (d, J = 2.2 Hz, | | | | | | |
| | 1H), 8.05-7.97 (m, 2H), 7.88 | | | | | | |
| | (ddd, J = 9.2, 8.2, 2.7 Hz, 1H). | | | | | | |
| 769 | 1H NMR (400 MHz, DMSO) δ 11.13 | DMSO | >98 | D, F3, H1 | | | |
| | (s, 1H), 9.58-9.54 (m, 1H), 9.51 | | | | | | |
| | (d, J = 2.2 Hz, 1H), 9.09-9.03 (m, | | | | | | |
| | 1H), 8.94-8.88 (m, 2H), 8.80 (dd, | | | | | | |
| | J = 9.9, 2.7 Hz, 1H), 8.63-8.58 (m, | | | | | | |
| | 1H), 8.08 (dd, J = 9.2, 5.4 Hz, 1H), | | | | | | |
| | 7.98-7.90 (m, 2H), 2.57 (s, 3H). | | | | | | |
| 770 | 1H NMR (400 MHz, DMSO) δ 10.91 | DMSO | >98 | C1, E, F3, H1 | | | |
| | (s, 1H), 9.55-9.53 (m, 1H), 9.51 | | | | | | |
| | (d, J = 2.5 Hz, 1H), 8.95-8.88 (m, | | | | | | |
| | 2H), 8.83 (dd, J = 5.1, 1.6 Hz, 1H), | | | | | | |
| | 8.24 (d, J = 2.7 Hz, 1H), 7.96-7.90 | | | | | | |
| | (m, 2H), 7.80 (dd, J = 8.0, 5.1 Hz, | | | | | | |
| | 1H), 7.63 (dd, J = 9.1, 2.6 Hz, 1H), | | | | | | |
| | 4.31 (q, J = 6.9 Hz, 2H), 2.73 (s, | | | | | | |
| | 3H), 1.46 (t, J = 6.9 Hz, 3H). | | | | | | |
| 771 | 1H NMR (400 MHz, DMSO) δ 10.99 | DMSO | >98 | D, F3, H1 | | | |
| | (s, 1H), 9.56 (d, J = 2.1 Hz, 1H), | | | | | | |
| | 9.51-9.46 (m, 1H), 8.98-8.91 | | | | | | |
| | (m, 2H), 8.89-8.81 (m, 2H), 8.04- | | | | | | |
| | 7.98 (m, 2H), 7.95 (d, J = 8.6 Hz, | | | | | | |
| | 1H), 7.84-7.77 (m, 1H), 2.75 (s, | | | | | | |
| | 3H). | | | | | | |
| 772 | 1H NMR (400 MHz, DMSO) δ 10.79 | DMSO | >98 | C1, E, F3, H1 | | | |
| | (s, 1H), 9.50 (d, J = 1.9 Hz, 1H), | | | | | | |
| | 9.14 (d, J = 2.1 Hz, 1H), 9.11-9.06 | | | | | | |
| | (m, 1H), 8.97 (dd, J = 5.3, 1.5 Hz, | | | | | | |
| | 1H), 8.71 (d, J = 9.2 Hz, 1H), 8.56- | | | | | | |
| | 8.53 (m, 1H), 8.48 (d, J = 2.2 Hz, | | | | | | |
| | 1H), 8.04 (dd, J = 8.1, 5.4 Hz, 1H), | | | | | | |
| | 7.47 (d, J = 2.6 Hz, 1H), 7.40 (dd, | | | | | | |
| | J = 9.1, 2.5 Hz, 1H), 3.99 (s, 3H). | | | | | | |
| 773 | 1H NMR (400 MHz, DMSO) δ 11.7 | DMSO | >98 | D, F3, H1 | | | |
| | (brs, 1H), 9.57 (d, J = 1.5 Hz, 1H), | | | | | | |
| | 8.98-8.90 (m, 2H), 8.49-8.43 | | | | | | |
| | (m, 1H), 8.42-8.27 (m, 2H), 8.09 | | | | | | |
| | (d, J = 9.1 Hz, 1H), 7.98 (d, J = | | | | | | |
| | 8.4 Hz, 1H), 7.71 (dd, J = 9.2, 2.6 Hz, | | | | | | |
| | 1H), 4.31 (q, J = 6.9 Hz, 2H), 2.40 | | | | | | |
| | (s, 3H), 1.46 (t, J = 6.9 Hz, 3H). | | | | | | |
| 774 | 1H NMR (400 MHz, DMSO) δ 10.63 | DMSO | >98 | D, F3, H1 | | | |
| | (s, 1H), 9.51 (d, J = 1.9 Hz, 1H), | | | | | | |
| | 9.12 (d, J = 2.1 Hz, 1H), 9.01-8.96 | | | | | | |
| | (m, 1H), 8.91 (dd, J = 5.3, 1.6 Hz, | | | | | | |
| | 1H), 8.72 (d, J = 8.9 Hz, 1H), 8.58- | | | | | | |
| | 8.54 (m, 1H), 8.48 (d, J = 2.2 Hz, | | | | | | |
| | 1H), 8.03 (d, J = 2.2 Hz, 1H), 7.95 | | | | | | |
| | (dd, J = 8.1, 5.2 Hz, 1H), 7.83 (dd, | | | | | | |
| | J = 8.9, 2.2 Hz, 1H). | | | | | | |
| 775 | 1H NMR (400 MHz, DMSO) δ 10.92 | DMSO | >98 | C1, E, F3, H1 | | | |
| | (s, 1H), 9.56 (d, J = 1.9 Hz, 1H), | | | | | | |
| | 9.15 (d, J = 8.2 Hz, 1H), 8.94 (d, | | | | | | |
| | J = 5.4 Hz, 1H), 8.55 (dd, J = 2.8, | | | | | | |
| | 0.7 Hz, 1H), 8.51-8.43 (m, 1H), 8.20 | | | | | | |
| | (d, J = 2.6 Hz, 1H), 8.09-7.98 (m, | | | | | | |
| | 2H), 7.94 (d, J = 9.0 Hz, 1H), 7.59 | | | | | | |
| | (dd, J = 9.1, 2.5 Hz, 1H), 4.29 (q, | | | | | | |
| | J = 6.9 Hz, 2H), 1.45 (t, J = 6.9 Hz, | | | | | | |
| | 3H). | | | | | | |
| 776 | 1H NMR (400 MHz, DMSO) δ 11.51 | DMSO | >98 | C1, E, F3, H1 | | | |
| | (brs, 1H), 9.57-9.53 (m, 1H), 9.06 | | | | | | |
| | (d, J = 8.1 Hz, 1H), 8.92 (dd, J = | | | | | | |
| | 5.3, 1.5 Hz, 1H), 8.37-8.28 (m, | | | | | | |
| | 2H), 8.05 (d, J = 8.4 Hz, 1H), 8.00- | | | | | | |
| | 7.92 (m, 2H), 7.64 (dd, J = 9.1, | | | | | | |
| | 2.6 Hz, 1H), 4.34 (q, J = 6.9 Hz, 2H), | | | | | | |
| | 2.61 (s, 3H), 2.36 (s, 3H), 1.45 (t, | | | | | | |
| | J = 6.9 Hz, 3H). | | | | | | |
| 777 | 1H NMR (400 MHz, DMSO) δ 10.06 | DMSO | >98 | D, F3, H1 | | | |
| | (s, 1H), 9.55 (d, J = 1.5 Hz, 1H), | | | | | | |
| | 9.06-9.03 (m, 1H), 8.87-8.81 | | | | | | |
| | (m, 2H), 8.75 (d, J = 2.5 Hz, 1H), | | | | | | |
| | 8.35 (d, J = 2.6 Hz, 1H), 8.01 (d, J = | | | | | | |
| | 2.6 Hz, 1H), 7.94 (d, J = 9.1 Hz, | | | | | | |
| | 1H), 7.62 (dd, J = 9.1, 2.5 Hz, 1H), | | | | | | |
| | 4.27 (q, J = 6.9 Hz, 2H), 1.47 (t, J = | | | | | | |
| | 6.9 Hz, 3H). | | | | | | |
| 778 | 1H NMR (400 MHz, DMSO) δ 10.03 | DMSO | >98 | D, F3, H1 | | | |
| | (s, 1H), 9.55 (d, J = 1.5 Hz, 1H), | | | | | | |
| | 9.20 (d, J = 2.2 Hz, 1H), 8.97-8.94 | | | | | | |
| | (m, 1H), 8.83 (dd, J = 2.5, 1.5 Hz, | | | | | | |
| | 1H), 8.75 (d, J = 2.5 Hz, 1H), 8.39 | | | | | | |
| | (d, J = 2.2 Hz, 1H), 8.00 (d, J = | | | | | | |
| | 2.7 Hz, 1H), 7.94 (d, J = 9.1 Hz, 1H), | | | | | | |
| | 7.65-7.60 (m, 1H), 4.27 (q, J = | | | | | | |
| | 6.9 Hz, 2H), 1.47 (t, J = 6.9 Hz, | | | | | | |
| | 3H). | | | | | | |
| 779 | 1H NMR (400 MHz, DMSO) δ 10.69 | DMSO | >98 | D, F3, H1 | | | |
| | (s, 1H), 9.53 (d, J = 1.9 Hz, 1H), | | | | | | |
| | 9.17 (d, J = 8.1 Hz, 1H), 8.96 (d, | | | | | | |
| | J = 5.4 Hz, 1H), 8.51 (d, J = 3.1 Hz, | | | | | | |
| | 1H), 8.39 (dd, J = 9.1, 4.1 Hz, 1H), | | | | | | |
| | 8.29 (dd, J = 8.4, 1.1 Hz, 1H), 8.10- | | | | | | |
| | 8.02 (m, 1H), 7.91 (ddd, J = 9.1, | | | | | | |
| | 8.2, 3.1 Hz, 1H), 7.66-7.60 (m, | | | | | | |
| | 1H), 7.47 (dd, J = 8.1, 1.0 Hz, 1H), | | | | | | |
| | 4.03 (s, 3H). | | | | | | |
| 780 | 1H NMR (400 MHz, DMSO) δ 10.76 | DMSO | >98 | D, F3, H1 | | | |
| | (s, 1H), 9.55 (d, J = 1.9 Hz, 1H), | | | | | | |
| | 9.25-9.20 (m, 1H), 9.02-8.97 | | | | | | |
| | (m, 1H), 8.54 (dd, J = 2.8, 0.6 Hz, | | | | | | |
| | 1H), 8.42 (dd, J = 8.9, 0.7 Hz, 1H), | | | | | | |
| | 8.30 (dd, J = 8.5, 1.1 Hz, 1H), 8.11 | | | | | | |
| | (dd, J = 8.1, 5.6 Hz, 1H), 8.06 (dd, | | | | | | |
| | J = 8.9, 2.7 Hz, 1H), 7.67-7.60 (m, | | | | | | |
| | 1H), 7.47 (dd, J = 8.1, 1.0 Hz, 1H), | | | | | | |
| | 4.03 (s, 3H). | | | | | | |
| 781 | 1H NMR (400 MHz, DMSO) δ 11.29 | DMSO | >98 | Method C2, | | | |
| | (s, 1H), 9.60 (d, J = 2.5 Hz, 1H), | | | E(NaOH_{(aq.)} | | | |
| | 9.56-9.53 (m, 1H), 9.11-9.05 | | | instead of | | | |
| | (m, 2H), 8.91 (dd, J = 5.4, 1.5 Hz, | | | NaOH and | | | |
| | 1H), 8.39 (d, J = 2.7 Hz, 1H), 8.00- | | | Δ), F2, H1 | | | |
| | 7.91 (m, 3H), 7.62 (dd, J = 9.1, | | | | | | |
| | 2.6 Hz, 1H), 4.03 (s, 3H), 2.77 (s, 3H). | | | | | | |
| 782 | 1H NMR (400 MHz, DMSO) δ 10.18 | DMSO | >98 | D, F3, H1 | | | |
| | (s, 1H), 9.50 (dd, J = 2.2, 0.9 Hz, | | | | | | |
| | 1H), 8.95-8.92 (m, 1H), 8.71 (dd, | | | | | | |
| | J = 4.8, 1.7 Hz, 1H), 8.66-8.59 (m, | | | | | | |
| | 2H), 8.27-8.24 (m, 1H), 8.18- | | | | | | |
| | 8.14 (m, 1H), 7.96 (d, J = 2.1 Hz, | | | | | | |
| | 1H), 7.74 (dd, J = 8.9, 2.2 Hz, 1H), | | | | | | |
| | 7.57 (ddd, J = 7.9, 4.8, 0.9 Hz, 1H), | | | | | | |
| | 2.40 (s, 3H). | | | | | | |
| 783 | 1H NMR (400 MHz, DMSO) δ 11.73 | DMSO | >98 | Method C2, | | | |
| | (s, 1H), 9.59 (d, J = 1.9 Hz, 1H), | | | E(NaOH_{(aq.)} | | | |
| | 9.22-9.15 (m, 1H), 8.97 (dd, J = | | | instead of | | | |
| | 5.5, 1.5 Hz, 1H), 8.52-8.46 (m, | | | NaOH and | | | |
| | 2H), 8.40 (d, J = 2.7 Hz, 1H), 8.15 | | | Δ), F2, H1 | | | |
| | (dd, J = 8.8, 2.3 Hz, 1H), 8.06 (dd, | | | | | | |
| | J = 8.1, 5.4 Hz, 1H), 8.00 (d, J = | | | | | | |
| | 9.1 Hz, 1H), 7.66 (dd, J = 9.1, 2.6 Hz, | | | | | | |
| | 1H), 4.02 (s, 3H), 2.75 (q, J = | | | | | | |
| | 7.6 Hz, 2H), 1.28 (t, J = 7.6 Hz, 3H). | | | | | | |
| 784 | 1H NMR (400 MHz, DMSO) δ 11.49 | DMSO | >98 | C1, E, F3, H1 | | | |
| | (s, 1H), 9.57 (d, J = 1.9 Hz, 1H), | | | | | | |
| | 9.18-9.12 (m, 1H), 8.95 (dd, J = | | | | | | |
| | 5.4, 1.5 Hz, 1H), 8.48-8.42 (m, | | | | | | |
| | 2H), 8.33 (d, J = 2.7 Hz, 1H), 8.09 | | | | | | |
| | (dd, J = 8.8, 2.3 Hz, 1H), 8.03 (dd, | | | | | | |
| | J = 8.1, 5.4 Hz, 1H), 7.98 (d, J = | | | | | | |
| | 9.1 Hz, 1H), 7.63 (dd, J = 9.1, 2.6 Hz, | | | | | | |
| | 1H), 4.30 (q, J = 6.9 Hz, 2H), 2.74 | | | | | | |
| | (q, J = 7.6 Hz, 2H), 1.45 (t, J = | | | | | | |
| | 6.9 Hz, 3H), 1.23 (t, J = 7.6 Hz, 3H). | | | | | | |
| 785 | 1H NMR (400 MHz, DMSO) δ 10.86 | DMSO | >98 | C1, E, F3, H1 | | | |
| | (s, 1H), 9.48 (d, J = 2.1 Hz, 1H), | | | | | | |
| | 9.15-9.08 (m, 1H), 9.03-8.93 | | | | | | |
| | (m, 2H), 8.47-8.39 (m, 1H), 8.25 | | | | | | |
| | (d, J = 2.7 Hz, 1H), 8.07 (dd, J = | | | | | | |
| | 8.2, 5.4 Hz, 1H), 7.96 (d, J = 9.1 Hz, | | | | | | |
| | 1H), 7.61 (dd, J = 9.2, 2.5 Hz, | | | | | | |
| | 1H), 4.30 (q, J = 6.9 Hz, 2H), 2.53 | | | | | | |
| | (d, J = 2.8 Hz, 3H), 1.45 (t, J = | | | | | | |
| | 6.9 Hz, 3H). | | | | | | |
| 786 | 1H NMR (400 MHz, DMSO) δ 9.67 | DMSO | >98 | C1, E, F3, H1 | | | |
| | (d, J = 2.0 Hz, 1H), 9.24 (d, J = 8.1 Hz, | | | | | | |
| | 1H), 8.94 (dd, J = 5.4, 1.6 Hz, | | | | | | |
| | 1H), 8.06-7.98 (m, 2H), 7.82 (brs, | | | | | | |
| | 1H), 7.68-7.62 (m, 2H), 7.49 (d, J = | | | | | | |
| | 2.8 Hz, 1H), 4.53 (t, J = 7.9 Hz, | | | | | | |
| | 2H), 4.04 (q, J = 6.9 Hz, 2H), 3.30- | | | | | | |
| | 3.23 (m, 2H), 2.26 (s, 3H), 1.36 (t, | | | | | | |
| | J = 6.9 Hz, 3H). | | | | | | |
| 787 | 1H NMR (400 MHz, DMSO) δ 11.29 | DMSO | >98 | D, F3, H1 | | | |
| | (s, 1H), 9.58 (d, J = 1.9 Hz, 1H), | | | | | | |
| | 9.24-9.11 (m, 1H), 8.98 (dd, J = | | | | | | |
| | 5.5, 1.5 Hz, 1H), 8.78 (dd, J = 9.8, | | | | | | |
| | 2.7 Hz, 1H), 8.48-8.40 (m, 1H), | | | | | | |
| | 8.33 (d, J = 8.6 Hz, 1H), 8.15-7.99 | | | | | | |
| | (m, 3H), 7.98-7.88 (m, 1H), 2.40 | | | | | | |
| | (s, 3H). | | | | | | |

TABLE 28

| | Starting | Starting | | | | | Salt |
|---|---|---|---|---|---|---|---|
| Number | Material 1 | Material 2 | | Product | | | type |
| 788 | 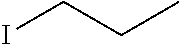 | 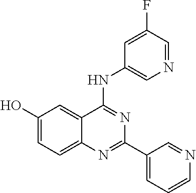 [SMILES: Oc1ccc2nc(-c3cccnc3)nc(Nc3cncc(F)c3)c2c1] | | 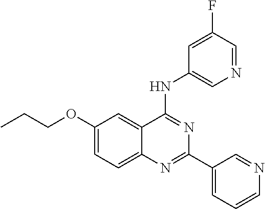 [SMILES: CCCOc1ccc2nc(-c3cccnc3)nc(Nc3cncc(F)c3)c2c1] | | | HCl |
| 789 | 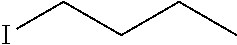 | 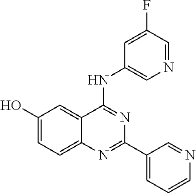 [SMILES: Oc1ccc2nc(-c3cccnc3)nc(Nc3cncc(F)c3)c2c1] | | 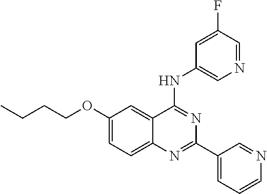 [SMILES: CCCCOc1ccc2nc(-c3cccnc3)nc(Nc3cncc(F)c3)c2c1] | | | HCl |
| 790 | 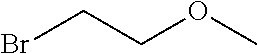 | 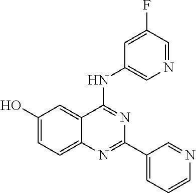 [SMILES: Oc1ccc2nc(-c3cccnc3)nc(Nc3cncc(F)c3)c2c1] | | 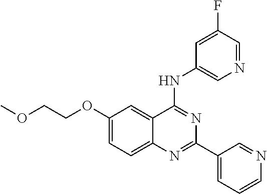 [SMILES: COCCOc1ccc2nc(-c3cccnc3)nc(Nc3cncc(F)c3)c2c1] | | | HCl |
| 791 | 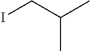 | 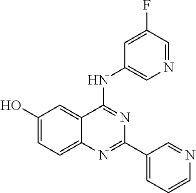 [SMILES: Oc1ccc2nc(-c3cccnc3)nc(Nc3cncc(F)c3)c2c1] | | 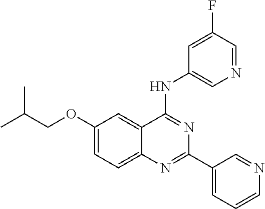 [SMILES: CC(C)COc1ccc2nc(-c3cccnc3)nc(Nc3cncc(F)c3)c2c1] | | | HCl |
| 792 | 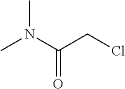 | 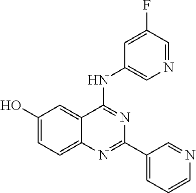 [SMILES: Oc1ccc2nc(-c3cccnc3)nc(Nc3cncc(F)c3)c2c1] | | 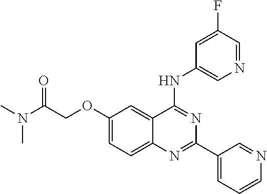 [SMILES: CN(C)C(=O)COc1ccc2nc(-c3cccnc3)nc(Nc3cncc(F)c3)c2c1] | | | HCl |
| 793 | 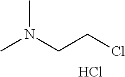 | 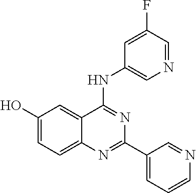 [SMILES: Oc1ccc2nc(-c3cccnc3)nc(Nc3cncc(F)c3)c2c1] | | 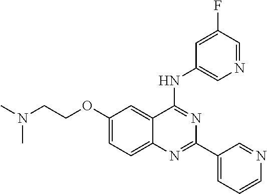 [SMILES: CN(C)CCOc1ccc2nc(-c3cccnc3)nc(Nc3cncc(F)c3)c2c1] | | | 3HCl |
| 794 | 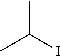 | 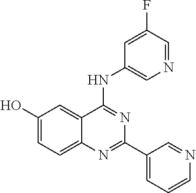 [SMILES: Oc1ccc2nc(-c3cccnc3)nc(Nc3cncc(F)c3)c2c1] | | 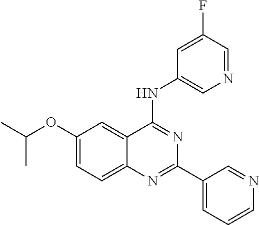 [SMILES: CC(C)Oc1ccc2nc(-c3cccnc3)nc(Nc3cncc(F)c3)c2c1] | | | HCl |
| 795 | 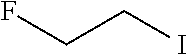 | 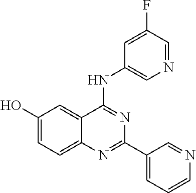 [SMILES: Oc1ccc2nc(-c3cccnc3)nc(Nc3cncc(F)c3)c2c1] | | 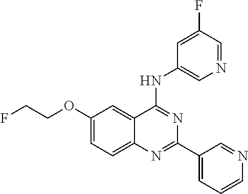 [SMILES: FCCOc1ccc2nc(-c3cccnc3)nc(Nc3cncc(F)c3)c2c1] | | | HCl |
| 796 | 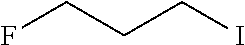 | 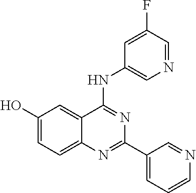 [SMILES: Oc1ccc2nc(-c3cccnc3)nc(Nc3cncc(F)c3)c2c1] | | 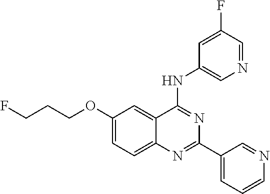 [SMILES: FCCCOc1ccc2nc(-c3cccnc3)nc(Nc3cncc(F)c3)c2c1] | | | HCl |
| | | ¹H NMR | Purity | Method | | Retention | LCMS |
| Number | ¹H NMR | Solvent | percent | of Coupling | LCMS | Time | Method |
| 788 | 1H NMR (400 MHz, DMSO) δ 10.47 | DMSO | >98 | M, N | | | |
| | (s, 1H), 9.47 (d, J = 2.0 Hz, 1H), | | | | | | |
| | 8.98 (d, J = 7.9 Hz, 1H), 8.91-8.86 | | | | | | |
| | (m, 1H), 8.75-8.71 (m, 1H), 8.48 | | | | | | |
| | (ddd, J = 8.8, 7.3, 2.8 Hz, 1H), 8.15- | | | | | | |
| | 8.09 (m, 1H), 7.98-7.89 (m, | | | | | | |
| | 2H), 7.63 (dd, J = 9.1, 2.6 Hz, 1H), | | | | | | |
| | 7.35 (dd, J = 8.8, 3.3 Hz, 1H), 4.18 | | | | | | |
| | (t, J = 6.5 Hz, 2H), 1.92-1.81 (m, | | | | | | |
| | 2H), 1.07 (t, J = 7.4 Hz, 3H). | | | | | | |
| 789 | 1H NMR (400 MHz, DMSO) δ 10.53 | DMSO | >98 | M, N | | | |
| | (s, 1H), 9.50-9.45 (m, 1H), 9.02- | | | | | | |
| | 8.96 (m, 1H), 8.89 (dd, J = 5.3, | | | | | | |
| | 1.5 Hz, 1H), 8.73 (dd, J = 2.8, 1.3 Hz, | | | | | | |
| | 1H), 8.49 (ddd, J = 8.9, 7.3, 2.8 Hz, | | | | | | |
| | 1H), 8.14 (d, J = 2.7 Hz, 1H), 7.98- | | | | | | |
| | 7.90 (m, 2H), 7.62 (dd, J = 9.1, 2.6 Hz, | | | | | | |
| | 1H), 7.34 (dd, J = 8.7, 3.2 Hz, | | | | | | |
| | 1H), 4.25-4.20 (m, 2H), 1.87- | | | | | | |
| | 1.78 (m, 2H), 1.57-1.47 (m, 2H), | | | | | | |
| | 0.99 (t, J = 7.4 Hz, 3H). | | | | | | |
| 790 | 1H NMR (400 MHz, DMSO) δ 10.56 | DMSO | >98 | M, N | | | |
| | (s, 1H), 9.51-9.44 (m, 1H), 9.03- | | | | | | |
| | 8.97 (m, 1H), 8.89 (dd, J = 5.3, | | | | | | |
| | 1.5 Hz, 1H), 8.78-8.71 (m, 1H), 8.50 | | | | | | |
| | (ddd, J = 8.8, 7.3, 2.8 Hz, 1H), 8.19 | | | | | | |
| | (d, J = 2.7 Hz, 1H), 8.00-7.91 (m, | | | | | | |
| | 2H), 7.65 (dd, J = 9.1, 2.6 Hz, 1H), | | | | | | |
| | 7.34 (dd, J = 8.8, 3.2 Hz, 1H), 4.38- | | | | | | |
| | 4.33 (m, 2H), 3.82-3.77 (m, | | | | | | |
| | 2H), 3.37 (s, 3H). | | | | | | |
| 791 | 1H NMR (400 MHz, DMSO) δ 10.40 | DMSO | >98 | M, N (50° C.) | | | |
| | (s, 1H), 9.49-9.45 (m, 1H), 8.98- | | | | | | |
| | 8.92 (m, 1H), 8.86 (dd, J = 5.2, 1.6 Hz, | | | | | | |
| | 1H), 8.74-8.70 (m, 1H), 8.48 | | | | | | |
| | (ddd, J = 8.9, 7.3, 2.8 Hz, 1H), 8.07 | | | | | | |
| | (d, J = 2.7 Hz, 1H), 7.96-7.85 (m, | | | | | | |
| | 2H), 7.64 (dd, J = 9.1, 2.6 Hz, 1H), | | | | | | |
| | 7.35 (dd, J = 8.8, 3.2 Hz, 1H), 4.01- | | | | | | |
| | 3.98 (m, 2H), 2.20-2.09 (m, | | | | | | |
| | 1H), 1.08 (d, J = 6.7 Hz, 6H). | | | | | | |
| 792 | 1H NMR (400 MHz, DMSO) δ 10.52 | DMSO | >98 | M, N (60° C.) | | | |
| | (s, 1H), 9.50-9.45 (m, 1H), 8.97- | | | | | | |
| | 8.90 (m, 1H), 8.88-8.83 (m, 1H), | | | | | | |
| | 8.78-8.74 (m, 1H), 8.55-8.47 | | | | | | |
| | (m, 1H), 8.24-8.16 (m, 1H), 7.97- | | | | | | |
| | 7.85 (m, 2H), 7.67 (dd, J = 9.1, | | | | | | |
| | 2.6 Hz, 1H), 7.34 (dd, J = 8.8, 3.2 Hz, | | | | | | |
| | 1H), 5.10 (s, 2H), 3.08 (s, 3H), | | | | | | |
| | 2.89 (s, 3H). | | | | | | |
| 793 | 1H NMR (400 MHz, DMSO) δ 11.00 | DMSO | >98 | M, N (60° C.) | | | |
| | (s, 1H), 10.58 (s, 1H), 9.51-9.47 | | | | | | |
| | (m, 1H), 9.06-9.00 (m, 1H), 8.91 | | | | | | |
| | (dd, J = 5.4, 1.5 Hz, 1H), 8.84- | | | | | | |
| | 8.81 (m, 1H), 8.62-8.52 (m, 2H), | | | | | | |
| | 8.03-7.93 (m, 2H), 7.67 (dd, J = | | | | | | |
| | 9.1, 2.6 Hz, 1H), 7.34 (dd, J = 8.8, | | | | | | |
| | 3.2 Hz, 1H), 4.68 (t, J = 5.2 Hz, | | | | | | |
| | 2H), 3.68-3.61 (m, 2H), 2.92 (s, | | | | | | |
| | 3H), 2.91 (s, 3H). | | | | | | |
| 794 | 1H NMR (400 MHz, DMSO) δ 10.50 | DMSO | >98 | M, N (60° C.) | | | |
| | (s, 1H), 9.49-9.45 (m, 1H), 9.00- | | | | | | |
| | 8.94 (m, 1H), 8.90-8.85 (m, 1H), | | | | | | |
| | 8.75-8.70 (m, 1H), 8.51-8.45 | | | | | | |
| | (m, 1H), 8.18-8.10 (m, 1H), 7.98- | | | | | | |
| | 7.88 (m, 2H), 7.61 (dd, J = 9.1, | | | | | | |
| | 2.5 Hz, 1H), 7.35 (dd, J = 8.8, | | | | | | |
| | 3.2 Hz, 1H), 5.00-4.92 (m, 1H), 1.40 | | | | | | |
| | (d, J = 6.0 Hz, 6H). | | | | | | |
| 795 | 1H NMR (400 MHz, DMSO) δ 10.39 | DMSO | >98 | M, N (60° C.) | | | |
| | (s, 1H), 9.50-9.47 (m, 1H), 8.97- | | | | | | |
| | 8.92 (m, 1H), 8.85 (dd, J = 5.3, 1.6 Hz, | | | | | | |
| | 1H), 8.75-8.72 (m, 1H), 8.49 | | | | | | |
| | (ddd, J = 8.9, 7.3, 2.9 Hz, 1H), 8.16 | | | | | | |
| | (d, J = 2.7 Hz, 1H), 7.95 (d, J = 9.1 Hz, | | | | | | |
| | 1H), 7.88 (dd, J = 8.1, 5.2 Hz, | | | | | | |
| | 1H), 7.68 (dd, J = 9.1, 2.6 Hz, 1H), | | | | | | |
| | 7.35 (dd, J = 8.8, 3.2 Hz, 1H), 4.96- | | | | | | |
| | 4.92 (m, 1H), 4.84-4.81 (m, | | | | | | |
| | 1H), 4.55-4.51 (m, 1H), 4.48- | | | | | | |
| | 4.44 (m, 1H). | | | | | | |
| 796 | 1H NMR (400 MHz, DMSO) δ 10.43 | DMSO | >98 | M, N (60° C.) | | | |
| | (s, 1H), 9.48 (d, J = 2.0 Hz, 1H), | | | | | | |
| | 8.99-8.94 (m, 1H), 8.87 (d, J = | | | | | | |
| | 5.1 Hz, 1H), 8.74-8.70 (m, 1H), | | | | | | |
| | 8.48 (ddd, J = 8.8, 7.3, 2.8 Hz, 1H), | | | | | | |
| | 8.13 (s, 1H), 7.97-7.87 (m, 2H), | | | | | | |
| | 7.64 (dd, J = 9.1, 2.6 Hz, 1H), 7.35 | | | | | | |
| | (dd, J = 8.8, 3.2 Hz, 1H), 4.76 (t, J = | | | | | | |
| | 5.8 Hz, 1H), 4.64 (t, J = 5.8 Hz, | | | | | | |
| | 1H), 4.33 (t, J = 6.3 Hz, 2H), 2.32- | | | | | | |
| | 2.18 (m, 2H). | | | | | | |

TABLE 29

| | | | | | | | Puri- | Method | | | |
|---|---|---|---|---|---|---|---|---|---|---|---|
| | | | | | | ¹H | ty | of | | Reten- | LCMS |
| Num- | Starting | Starting | | Salt | | NMR | per- | Cou- | | tion | Meth- |
| ber | Material 1 | Material 2 | Product | type | ¹H NMR | Solvent | cent | pling | LCMS | Time | od |
| 797 | 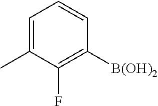 | 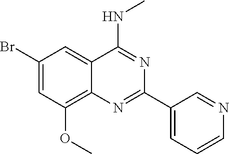 [SMILES: CNc1nc(-c2cccnc2)nc2c(OC)cc(Br)cc12] | 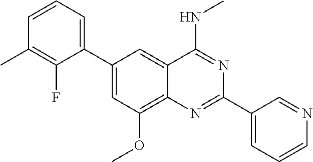 [SMILES: CNc1nc(-c2cccnc2)nc2c(OC)cc(-c3cccc(C)c3F)cc12] | HCl | ¹H NMR (400 MHz, DMSO) δ 9.60 (m, 1H), 9.10 (m, 1H), 8.97 (m, 1H), 8.90 (dd, J = 5.3, 1.6 Hz, 1H), 8.04 (m, 1H), 7.93 (dd, J = 7.8, 5.2 Hz, 1H), 7.55-7.46 (m, 2H), 7.39 (m, 1H), 7.27 (dd, J = 7.6, 7.6 Hz, 1H), 4.05 (s, 3H), 3.19 (d, J = 4.5 Hz, 3H), 2.35 (d, J = 2.3 Hz, 3H). | DMSO | >98 | R3 Temper- ature at 100° C. | | | |
| 798 | 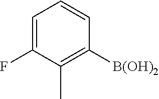 | 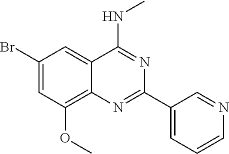 [SMILES: CNc1nc(-c2cccnc2)nc2c(OC)cc(Br)cc12] | 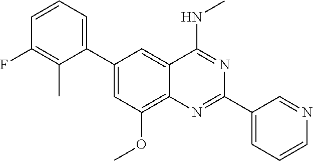 [SMILES: CNc1nc(-c2cccnc2)nc2c(OC)cc(-c3cccc(F)c3C)cc12] | HCl | ¹H NMR (400 MHz, DMSO) δ 9.59 (m, 1H), 9.12 (m, 1H), 8.99 (br-s, 1H), 8.92 (m, 1H), 7.96 (dd, J = 8.0, 5.3 Hz, 1H), 7.86 (m, 1H), 7.43-7.34 (m, 2H), 7.30-7.19 (m, 2H), 4.04 (s, 3H), 3.19 (d, J = 4.5 Hz, 3H), 2.23 (d, J = 2.5 Hz, 3H). | DMSO | >98 | R3 Temper- ature at 100° C. | | | |
| 799 | 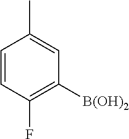 | 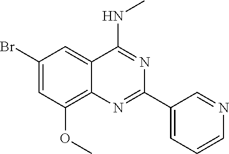 [SMILES: CNc1nc(-c2cccnc2)nc2c(OC)cc(Br)cc12] | 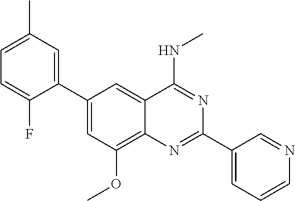 [SMILES: CNc1nc(-c2cccnc2)nc2c(OC)cc(-c3cc(C)ccc3F)cc12] | 2HCl | ¹H NMR (400 MHz, DMSO) δ 9.58 (m, 1H), 9.34-9.19 (m, 2H), 9.01 (dd, J = 5.5, 1.6 Hz, 1H), 8.17-8.05 (m, 2H), 7.61-7.48 (m, 2H), 7.37-7.22 (m, 2H), 4.06 (s, 3H), 3.21 (d, J = 4.3 Hz, 3H), 2.40 (s, 3H). | DMSO | >98 | R3 Temper- ature at 100° C. | | | |
| 800 | 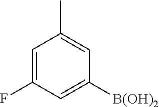 | 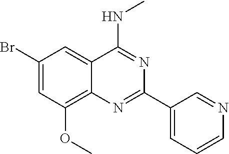 [SMILES: CNc1nc(-c2cccnc2)nc2c(OC)cc(Br)cc12] | 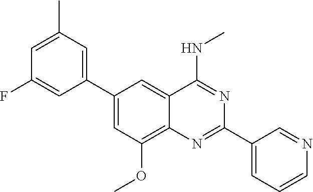 [SMILES: CNc1nc(-c2cccnc2)nc2c(OC)cc(-c3cc(C)cc(F)c3)cc12] | 2HCl | ¹H NMR (400 MHz, DMSO) δ 9.59 (d, J = 2.3 Hz, 2H), 9.15 (m, 1H), 8.94 (m, 1H), 8.24 (m, 1H), 7.99 (dd, = 8.5, 5.2 Hz, 1H), 7.69 (m, 1H), 7.65-7.55 (m, 2H), 7.13 (m, 1H), 4.12 (s, 3H), 3.23 (d, J = 4.5 Hz, 3H), 2.46 (s, 3H). | DMSO | >98 | R3 Temper- ature at 100° C. | | | |
| 801 | 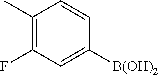 | 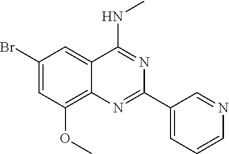 [SMILES: CNc1nc(-c2cccnc2)nc2c(OC)cc(Br)cc12] | 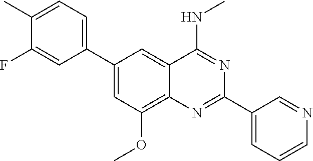 [SMILES: CNc1nc(-c2cccnc2)nc2c(OC)cc(-c3ccc(C)c(F)c3)cc12] | 2HCl | ¹H NMR (400 MHz, DMSO) δ 9.56 (d, J = 2.1 Hz, 1H), 9.32 (br-s, 1H), 9.18 (m, 1H), 8.97 (m, 1H), 8.30 (m, 1H), 8.05 (dd, J = 8.4, 5.3 Hz, 1H), 7.82-7.65 (m, 3H), 7.46 (dd, J = 8.1, 8.1 Hz, 1H), 4.12 (s, 3H), 3.23 (d, J = 4.5 Hz, 3H), 2.32 (d, J = 1.9 Hz, 3H). | DMSO | >98 | R3 Temper- ature at 100° C. | | | |
| 802 | 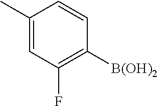 | 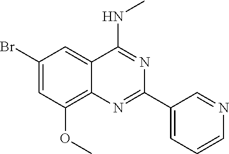 [SMILES: CNc1nc(-c2cccnc2)nc2c(OC)cc(Br)cc12] | 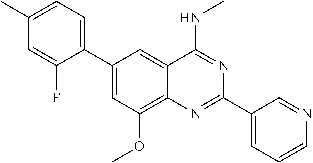 [SMILES: CNc1nc(-c2cccnc2)nc2c(OC)cc(-c3ccc(C)cc3F)cc12] | 2HCl | ¹H NMR (400 MHz, DMSO) δ 9.58 (d, J = 2.3 Hz, 1H), 9.37-9.11 (m, 2H), 8.99 (m, 1H), 8.17-8.00 (m, 2H), 7.62 (dd, J = 8.3, 8.3 Hz, 1H), 7.54 (m, 1H), 7.27-7.18 (m, 2H), 4.06 (s, 3H), 3.21 (d, J = 4.4 Hz, 3H), 2.41 (s, 3H). | DMSO | >98 | R3 Temper- ature at 100° C. | | | |
| 803 | 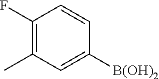 | 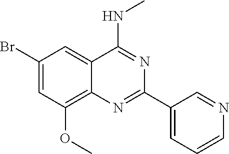 [SMILES: CNc1nc(-c2cccnc2)nc2c(OC)cc(Br)cc12] | 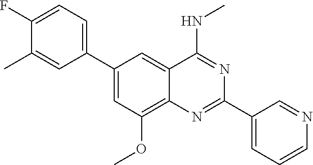 [SMILES: CNc1nc(-c2cccnc2)nc2c(OC)cc(-c3ccc(F)c(C)c3)cc12] | 2HCl | ¹H NMR (400 MHz, DMSO) δ 9.59-9.41 (m, 2H), 9.17 (ddd, J = 8.0, 1.8, 1.8 Hz, 1H), 8.99 (m, 1H), 8.28 (d, J = 1.9 Hz, 1H), 8.07 (dd, J = 8.2, 5.4 Hz, 1H), 7.89 (dd, J = 7.5, 2.7 Hz, 1H), 7.79 (m, 1H), 7.67 (m, 1H), 7.31 (dd, J = 9.1, 9.1 Hz, 1H), 4.12 (s, 3H), 3.22 (d, J = 4.4 Hz, 3H), 2.36 (d, J = 2.1 Hz, 3H). | DMSO | >98 | R3 Temper- ature at 100° C. | | | |
| 804 | 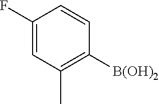 | 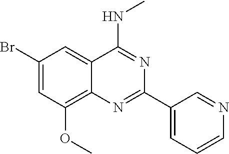 [SMILES: CNc1nc(-c2cccnc2)nc2c(OC)cc(Br)cc12] | 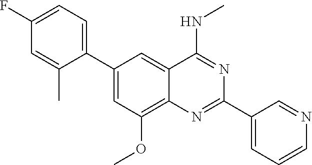 [SMILES: CNc1nc(-c2cccnc2)nc2c(OC)cc(-c3ccc(F)cc3C)cc12] | 2HCl | ¹H NMR (400 MHz, DMSO) δ 9.56 (d, J = 2.3 Hz, 1H), 9.36 (br-s, 1H), 9.21 (d, J = 8.0 Hz, 1H), 9.01 (m, 1H), 8.10 (m, 1H), 7.94 (m, 1H), 7.40 (m, 2H), 7.25 (dd, J = 10.2, 2.9 Hz, 1H), 7.17 (ddd, J = 8.6, 8.5, 3.0 Hz, 1H), 4.05 (s, 3H), 3.20 (d, J = 4.3 Hz, 3H), 2.35 (s, 3H). | DMSO | >98 | R3 Temper- ature at 100° C. | | | |
| 805 | 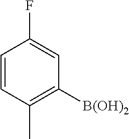 | 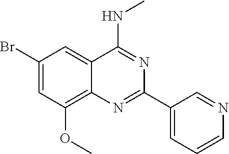 [SMILES: CNc1nc(-c2cccnc2)nc2c(OC)cc(Br)cc12] | 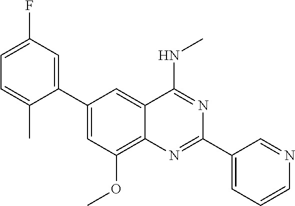 [SMILES: CNc1nc(-c2cccnc2)nc2c(OC)cc(-c3cc(F)ccc3C)cc12] | 2HCl | ¹H NMR (400 MHz, DMSO) δ 9.57 (m, 1H), 9.32-9.17 (m, 2H), 9.00 (m, 1H), 8.09 (dd, J = 8.2, 5.4 Hz, 1H), 7.95 (m, 1H), 7.46-7.35 (m, 2H), 7.27-7.15 (m, 2H), 4.06 (s, 3H), 3.20 (d, J = 4.4 Hz, 3H), 2.32 (s, 3H). | DMSO | >98 | R3 Temper- ature at 100° C. | | | |
| 806 | 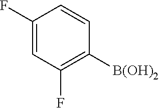 [SMILES: OB(O)c1ccc(F)cc1F] | 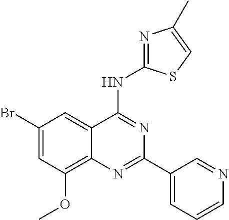 [SMILES: COc1cc(Br)cc2c(Nc3nc(C)cs3)nc(-c3cccnc3)nc12] | 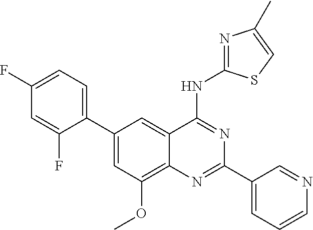 [SMILES: COc1cc(-c2ccc(F)cc2F)cc2c(Nc3nc(C)cs3)nc(-c3cccnc3)nc12] | 2HCl | ¹H NMR (400 MHz, DMSO) δ 9.75 (m, 1H), 9.25 (m, 1H), 8.93 (m, 1H), 8.47 (br- s, 1H), 8.01 (m, 1H), 7.84 (ddd, J = 9.0, 8.9, 6.7 Hz, 1H), 7.57 (m, 1H), 7.47 (ddd, J = 11.3, 9.2, 2.6 Hz, 1H), 7.31 (ddd, J = 8.4, 8.3, 2.8 Hz, 1H), 6.94 (s, 1H), 4.08 (s, 3H), 2.35 (s, 3H). | DMSO | >98 | R3 Temper- ature at 100° C. | | | |
| 807 | 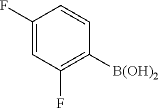 [SMILES: OB(O)c1ccc(F)cc1F] | 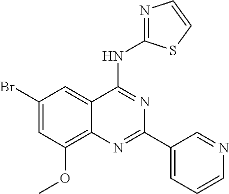 [SMILES: COc1cc(Br)cc2c(Nc3nccs3)nc(-c3cccnc3)nc12] | 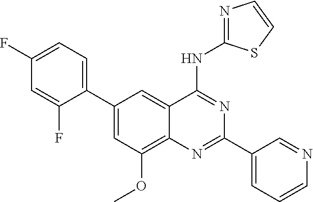 [SMILES: COc1cc(-c2ccc(F)cc2F)cc2c(Nc3nccs3)nc(-c3cccnc3)nc12] | | ¹H NMR (400 MHz, DMSO) δ 12.57 (br-s, 1H), 9.79 (dd, J = 2.2, 0.9 Hz, 1H), 8.93 (dt, J = 8.0, 1.9, 1.9 Hz, 1H), 8.75 (dd, J = 4.8, 1.8 Hz, 1H), 8.55 (br-s, 1H), 7.85 (m, 1H), 7.72-7.59 (m, 2H), 7.55 (s, 1H), 7.47 (m, 1H), 7.39 (br-s, 1H), 7.31 (ddd, J = 10.7, 7.8, 2.4 Hz, 1H), 4.07 (s, 3H). | DMSO | >98 | R3 Temper- ature at 100° C. | | | |
| 808 | 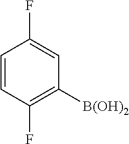 [SMILES: OB(O)c1cc(F)ccc1F] | 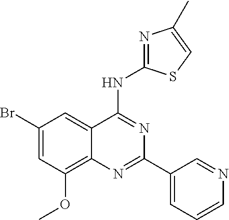 [SMILES: COc1cc(Br)cc2c(Nc3nc(C)cs3)nc(-c3cccnc3)nc12] | 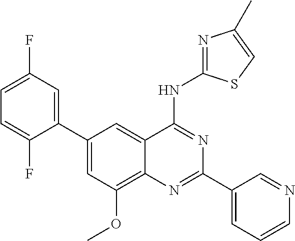 [SMILES: COc1cc(-c2cc(F)ccc2F)cc2c(Nc3nc(C)cs3)nc(-c3cccnc3)nc12] | | ¹H NMR (400 MHz, DMSO) δ 12.5 (br-s, 1H), 9.79 (dd, J = 2.2, 0.9 Hz, 1H), 8.91 (ddd, J = 8.0, 1.8, 1.8 Hz, 1H), 8.75 (dd, J = 4.8, 1.8 Hz, 1H), 8.56 (m, 1H), 7.82-7.26 (m, 5H), 6.92 (m, 1H), 4.08 (s, 3H), 2.35 (s, 3H). | DMSO | >98 | R3 Temper- ature at 100° C. | | | |
| 809 | 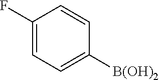 | 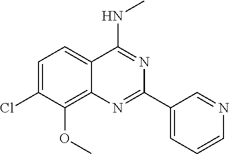 [SMILES: CNc1nc(-c2cccnc2)nc2c(OC)c(Cl)ccc12] | 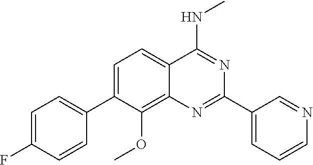 [SMILES: CNc1nc(-c2cccnc2)nc2c(OC)c(-c3ccc(F)cc3)ccc12] | 2HCl | ¹H NMR (400 MHz, DMSO) δ 9.66 (d, J = 2.3 Hz, 1H), 9.27 (m, 1H), 9.08-8.89 (m, 2H), 8.24-7.97 (m, 2H), 7.76-7.64 (m, 2H), 7.60 (d, J = 8.7 Hz, 1H), 7.43-7.28 (m, 2H), 3.99 (s, 3H), 3.22 (d, J = 4.5 Hz, 3H). | DMSO | >98 | R5 | | | |
| 810 | 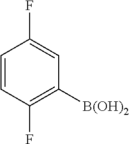 [SMILES: OB(O)c1cc(F)ccc1F] | 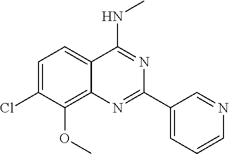 [SMILES: CNc1nc(-c2cccnc2)nc2c(OC)c(Cl)ccc12] | 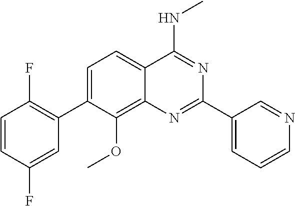 [SMILES: CNc1nc(-c2cccnc2)nc2c(OC)c(-c3cc(F)ccc3F)ccc12] | 2HCl | ¹H NMR (400 MHz, DMSO) δ 9.68 (d, J = 2.3 Hz, 1H), 9.25 (m, 1H), 9.03-8.78 (m, 2H), 8.19-7.92 (m, 2H), 7.52 (m, 1H), 7.46-7.30 (m, 3H), 4.10 (s, 3H), 3.21 (d, J = 4.4 Hz, 3H). | DMSO | >98 | R5 | | | |
| 811 | 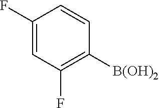 [SMILES: OB(O)c1ccc(F)cc1F] | 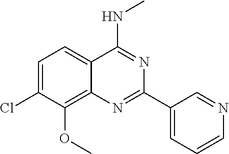 [SMILES: CNc1nc(-c2cccnc2)nc2c(OC)c(Cl)ccc12] | 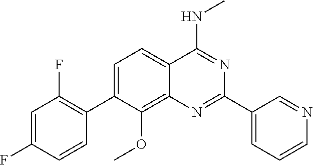 [SMILES: CNc1nc(-c2cccnc2)nc2c(OC)c(-c3ccc(F)cc3F)ccc12] | 2HCl | ¹H NMR (400 MHz, DMSO) δ 9.68 (d, J = 2.3 Hz, 1H), 9.18 (m, 1H), 8.99-8.68 (m, 2H), 8.16-7.86 (m, 2H), 7.61-7.34 (m, 3H), 7.24 (dd, J = 10.1, 7.1 Hz, 1H), 4.07 (s, 3H), 3.20 (d, J = 4.4 Hz, 3H) | DMSO | >98 | R5 | | | |
| 812 | 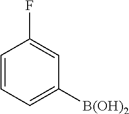 | 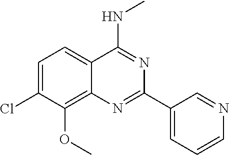 [SMILES: CNc1nc(-c2cccnc2)nc2c(OC)c(Cl)ccc12] | 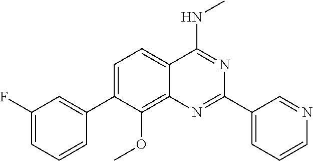 [SMILES: CNc1nc(-c2cccnc2)nc2c(OC)c(-c3cccc(F)c3)ccc12] | 2HCl | ¹H NMR (400 MHz, DMSO) δ 9.67(d, J = 2.0 Hz, 1H), 9.39-9.21 (m, 1H), 9.04-8.86 (m, 2H), 8.20-8.00 (m, 2H), 7.70-7.53 (m, 2H), 7.53-7.43 (m, 2H), 7.34-7.23 (m, 1H), 4.03 (s, 3H), 3.21 (d, J = 4.5 Hz, 3H). | DMSO | >98 | R5 | | | |
| 813 | 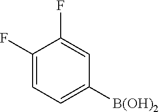 [SMILES: OB(O)c1ccc(F)c(F)c1] | 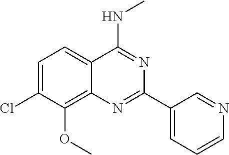 [SMILES: CNc1nc(-c2cccnc2)nc2c(OC)c(Cl)ccc12] | 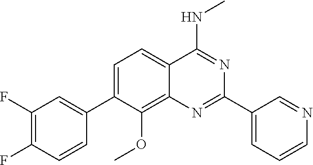 [SMILES: CNc1nc(-c2cccnc2)nc2c(OC)c(-c3ccc(F)c(F)c3)ccc12] | 2HCl | ¹H NMR (400 MHz, DMSO) δ 9.70 (s, 1H), 9.27 (m, 1H), 9.00-8.84 (m, 2H), 8.12 (d, J = 8.7 Hz, 1H), 8.05 (dd, J = 8.2, 5.3 Hz, 1H), 7.74 (m, 1H), 7.67-7.56 (m, 2H), 7.56-7.49 (m, 1H), 4.08 (s, 3H), 3.24 (d, J = 4.2 Hz, 3H). | DMSO | >98 | R5 | | | |
| 814 | 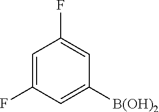 [SMILES: OB(O)c1cc(F)cc(F)c1] | 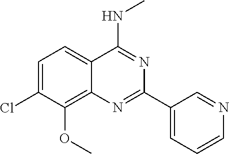 [SMILES: CNc1nc(-c2cccnc2)nc2c(OC)c(Cl)ccc12] | 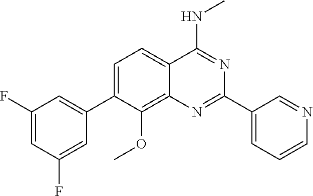 [SMILES: CNc1nc(-c2cccnc2)nc2c(OC)c(-c3cc(F)cc(F)c3)ccc12] | 2HCl | ¹H NMR (400 MHz, DMSO) δ 9.65 (d, J = 2.0 Hz, 1H), 9.40 (ddd, J = 8.2, 1.8, 1.8 Hz, 1H), 9.11 (br-s, 1H), 9.04 (dd, J = 5.7, 1.7 Hz, 1H), 8.23-8.13 (m, 2H), 7.64 (d, J = 8.7 Hz, 1H), 7.45-7.27 (m, 3H), 4.07 (s, 3H), 3.20 (d, J = 4.3 Hz, 3H). | DMSO | >98 | R5 | | | |
| 815 | 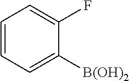 | 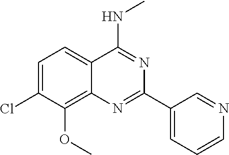 [SMILES: CNc1nc(-c2cccnc2)nc2c(OC)c(Cl)ccc12] | 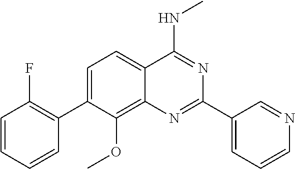 [SMILES: CNc1nc(-c2cccnc2)nc2c(OC)c(-c3ccccc3F)ccc12] | 3HCl | ¹H NMR (400 MHz, DMSO) δ 9.67 (d, J = 1.9 Hz, 1H), 9.36 (d, J = 7.9 Hz, 1H), 9.07-8.96 (m, 2H), 8.20-8.11 (m, 2H), 7.57-7.44 (m, 3H), 7.40-7.30 (m, 2H), 4.05 (s, 3H), 3.22 (d, J = 4.4 Hz, 3H). | DMSO | >98 | R5 | | | |
| 816 | 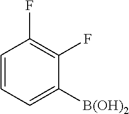 [SMILES: OB(O)c1cccc(F)c1F] | 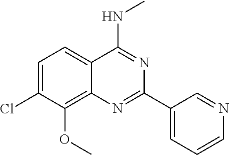 [SMILES: CNc1nc(-c2cccnc2)nc2c(OC)c(Cl)ccc12] | 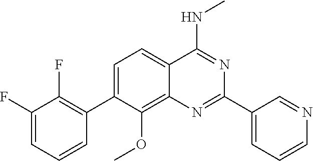 [SMILES: CNc1nc(-c2cccnc2)nc2c(OC)c(-c3cccc(F)c3F)ccc12] | 2HCl | ¹H NMR (400 MHz, DMSO) δ 9.63 (d, J = 2.1 Hz, 1H), 9.26 (m, 1H), 8.83 (dd, J = 5.5, 1.6 Hz, 1H), 8.74 (m, 1H), 8.04-7.94 (m, 2H), 7.52-7.44 (m, 2H), 7.39-7.22 (m, 2H), 3.19 (d, J = 4.0 Hz, 3H). MeO peak (3H) was overlapped with H2O peak around 4.0~4.2. | DMSO | >98 | R5 | | | |
| 817 | 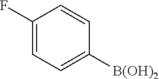 | 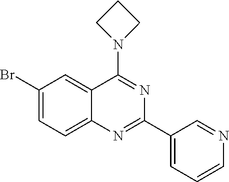 [SMILES: Brc1ccc2nc(-c3cccnc3)nc(N3CCC3)c2c1] | 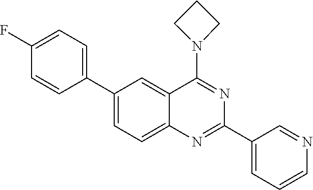 [SMILES: Fc1ccc(-c2ccc3nc(-c4cccnc4)nc(N4CCC4)c3c2)cc1] | 2HCl | ¹H NMR (400 MHz, DMSO) δ 9.58 (s, 1H), 9.03-8.85 (m, 2H), 8.41-8.21 (m, 2H), 8.15 (s, 1H), 7.99-7.75 (m, 3H), 7.49-7.28 (m, 2H), 5.44-4.51 (m, 4H), 2.69-2.52 (m, 2H). | DMSO | >98 | R3 Temper- ature at 100° C. | | | |
| 818 | 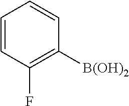 | 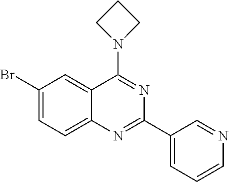 [SMILES: Brc1ccc2nc(-c3cccnc3)nc(N3CCC3)c2c1] | 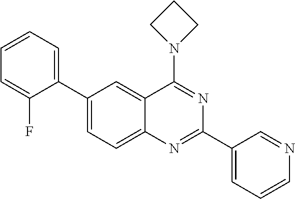 [SMILES: Fc1ccccc1-c1ccc2nc(-c3cccnc3)nc(N3CCC3)c2c1] | 2HCl | ¹H NMR (400 MHz, DMSO) δ 9.60 (s, 1H), 9.04-8.89 (m, 2H), 8.33-8.13 (m, 3H), 7.85 (m, 1H), 7.75 (ddd, J = 8.0, 7.9, 1.8 Hz, 1H), 7.53 (m, 1H), 7.46-7.34 (m, 2H), 5.33-4.35 (m, 4H), 2.63-2.53 (m, 2H). | DMSO | >98 | R3 Temper- ature at 100° C. | | | |
| 819 | 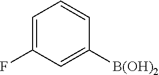 | 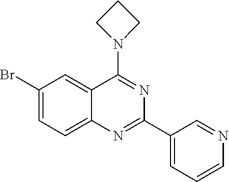 [SMILES: Brc1ccc2nc(-c3cccnc3)nc(N3CCC3)c2c1] | 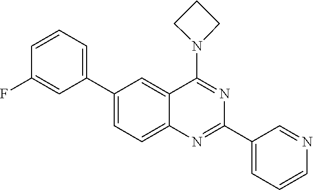 [SMILES: Fc1cccc(-c2ccc3nc(-c4cccnc4)nc(N4CCC4)c3c2)c1] | 2HCl | ¹H NMR (400 MHz, DMSO) δ 9.61 (d, J = 2.4 Hz, 1H), 9.07-8.89 (m, 2H), 8.40-8.29 (m, 2H), 8.18 (s, 1H), 7.91-7.53 (m, 4H), 7.30 (ddd, J = 8.5, 8.5, 2.8 Hz, 1H), 5.36-3.76 (m, 4H), 2.66-2.53 (m, 2H). | DMSO | >98 | R3 Temper- ature at 100° C. | | | |
| 820 | 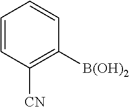 [SMILES: N#Cc1ccccc1B(O)O] | 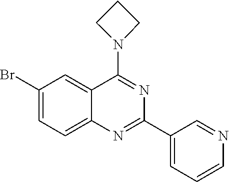 [SMILES: Brc1ccc2nc(-c3cccnc3)nc(N3CCC3)c2c1] | 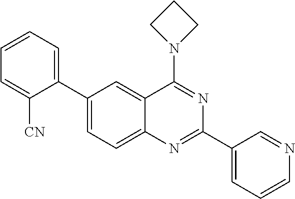 [SMILES: N#Cc1ccccc1-c1ccc2nc(-c3cccnc3)nc(N3CCC3)c2c1] | 2HCl | ¹H NMR (400 MHz, DMSO) δ 9.57 (s, 1H), 8.90-8.82 (m, 2H), 8.23-8.13 (m, 2H), 8.10-8.03 (m, 2H), 7.92-7.73 (m, 3H), 7.67 (m, 1H), 5.13-4.53 (m, 4H), 2.59-2.53 (m, 2H). | DMSO | >98 | R3 Temper- ature at 100° C. | | | |
| 821 | 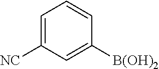 [SMILES: N#Cc1cccc(B(O)O)c1] | 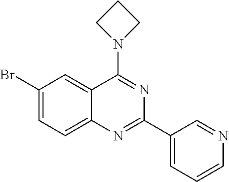 [SMILES: Brc1ccc2nc(-c3cccnc3)nc(N3CCC3)c2c1] | 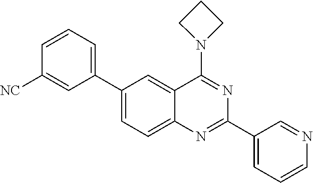 [SMILES: N#Cc1cccc(-c2ccc3nc(-c4cccnc4)nc(N4CCC4)c3c2)c1] | 2HCl | ¹H NMR (400 MHz, DMSO) δ 9.58 (d, J = 2.4 Hz, 1H), 8.98-8.90 (m, 2H), 8.43-8.35 (m, 2H), 8.27-8.16 (m, 3H), 7.93 (ddd, J = 7.8, 1.4, 1.4 Hz, 1H), 7.84 (dd, J = 8.1, 5.0 Hz, 1H), 7.75 (dd, J = 7.8, 7.8 Hz, 1H), 5.40-4.53 (m, 4H), 2.63-2.53 (m, 2H). | DMSO | >98 | R3 Temper- ature at 100° C. | | | |
| 822 | 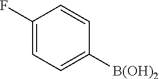 | 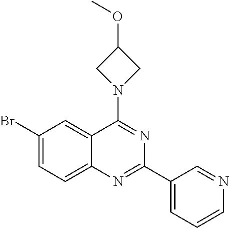 [SMILES: COC1CN(c2nc(-c3cccnc3)nc3ccc(Br)cc23)C1] | 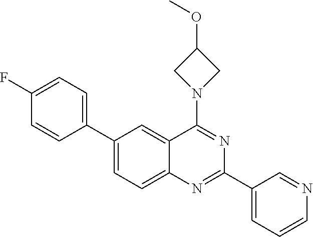 [SMILES: COC1CN(c2nc(-c3cccnc3)nc3ccc(-c4ccc(F)cc4)cc23)C1] | 2HCl | ¹H NMR (400 MHz, DMSO) δ 9.62 (dd, J = 2.3, 0.9 Hz, 1H), 9.05 (m, 1H), 8.95 (dd, J = 5.1, 1.6 Hz, 1H), 8.37-8.24 (m, 2H), 8.18 (d, J = 2.3 Hz, 1H), 7.97-7.83 (m, 3H), 7.43-7.33 (m, 2H), 5.54-4.41 (m, 5H), 3.37 (s, 3H). | DMSO | >98 | R3 Temper- ature at 100° C. | | | |
| 823 | 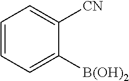 [SMILES: N#Cc1ccccc1B(O)O] | 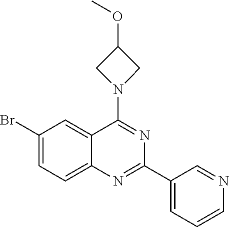 [SMILES: COC1CN(c2nc(-c3cccnc3)nc3ccc(Br)cc23)C1] | 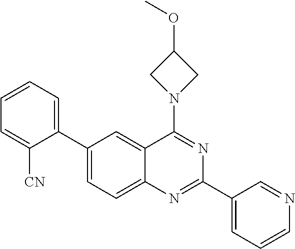 [SMILES: COC1CN(c2nc(-c3cccnc3)nc3ccc(-c4ccccc4C#N)cc23)C1] | 2HCl | ¹H NMR (400 MHz, DMSO) δ 9.61 (d, J = 2.0 Hz, 1H), 8.99 (m, 1H), 8.93 (dd, J = 5.0, 1.8 Hz, 1H), 8.26 (d, J = 2.1 Hz, 1H), 8.24-8.13 (m, 2H), 8.05 (m, 1H), 7.93-7.82 (m, 3H), 7.68 (ddd, J = 7.1, 7.0, 1.7 Hz, 1H), 5.31-4.32 (m, 5H), 3.34 (s, 3H). | DMSO | >98 | R3 Temper- ature at 100° C. | | | |
| 824 | 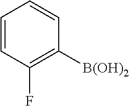 | 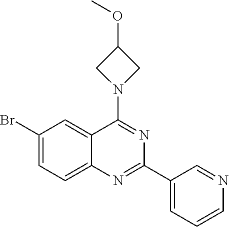 [SMILES: COC1CN(c2nc(-c3cccnc3)nc3ccc(Br)cc23)C1] | 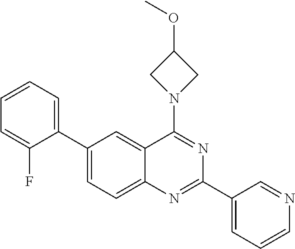 [SMILES: COC1CN(c2nc(-c3cccnc3)nc3ccc(-c4ccccc4F)cc23)C1] | 2HCl | ¹H NMR (400 MHz, DMSO) δ 9.63 (dd, J = 2.2, 0.8 Hz, 1H), 9.10-9.03 (m, 1H), 8.96 (dd, J = 5.1, 1.6 Hz, 1H), 8.32-8.14 (m, 3H), 7.90 (m, 1H), 7.78 (ddd, J = 7.9, 7.9, 1.7 Hz, 1H), 7.53 (m, 1H), 7.47-7.35 (m, 2H), 5.34-3.94 (m, 5H), 3.35 (s, 3H). | DMSO | >98 | R3 Temper- ature at 100° C. | | | |
| 825 | 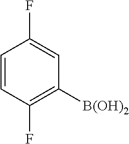 [SMILES: OB(O)c1cc(F)ccc1F] | 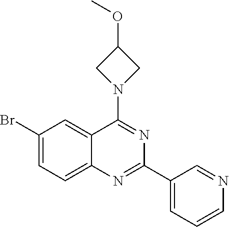 [SMILES: COC1CN(c2nc(-c3cccnc3)nc3ccc(Br)cc23)C1] | 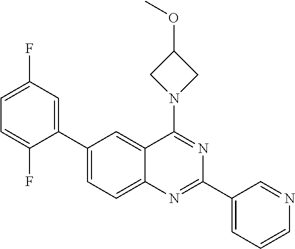 [SMILES: COC1CN(c2nc(-c3cccnc3)nc3ccc(-c4cc(F)ccc4F)cc23)C1] | 2HCl | ¹H NMR (400 MHz, DMSO) δ 9.65 (dd, J = 2.3, 0.8 Hz, 1H), 9.16-9.09 (m, 1H), 8.97 (dd, J = 5.1, 1.5 Hz, 1H), 8.31 (d, J = 8.7 Hz, 1H), 8.25-8.16 (m, 2H), 7.92 (dd, J = 8.0, 5.1 Hz, 1H), 7.71 (m, 1H), 7.47 (ddd, J = 10.2, 9.1, 4.7 Hz, 2H), 7.37 (m, 1H), 5.36-3.94 (m, 5H), 3.35 (s, 3H). | DMSO | >98 | R3 Temper- ature at 100° C. | | | |
| 826 | 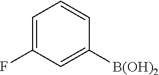 | 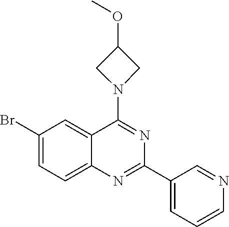 [SMILES: COC1CN(c2nc(-c3cccnc3)nc3ccc(Br)cc23)C1] | 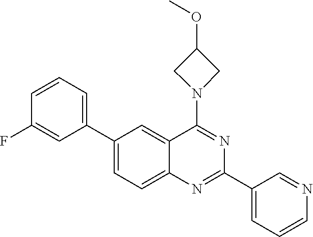 [SMILES: COC1CN(c2nc(-c3cccnc3)nc3ccc(-c4cccc(F)c4)cc23)C1] | 2HCl | ¹H NMR (400 MHz, DMSO) δ 9.65 (m, 1H), 9.11 (ddd, J = 8.2, 1.9, 1.9 Hz, 1H), 8.96 (dd, J = 5.1, 1.5 Hz, 1H), 8.39-8.29 (m, 2H), 8.20 (dd, J = 1.3, 1.3 Hz, 1H), 7.90 (dd, J = 8.0, 5.1 Hz, 1H), 7.79-7.68 (m, 2H), 7.58 (ddd, J = 8.0, 8.0, 6.1 Hz, 1H), 7.30 (m, 1H), 5.51-4.44 (m, 5H), 3.38 (s, 3H). | DMSO | >98 | R3 Temper- ature at 100° C. | | | |
| 827 | 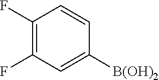 [SMILES: OB(O)c1ccc(F)c(F)c1] | 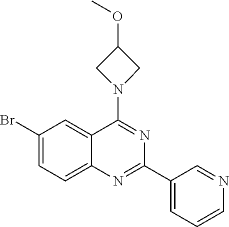 [SMILES: COC1CN(c2nc(-c3cccnc3)nc3ccc(Br)cc23)C1] | 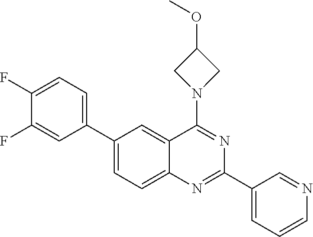 [SMILES: COC1CN(c2nc(-c3cccnc3)nc3ccc(-c4ccc(F)c(F)c4)cc23)C1] | 2HCl | ¹H NMR (400 MHz, DMSO) δ 9.61 (d, J = 2.3 Hz, 1H), 9.04 (m, 1H), 8.94 (dd, J = 5.0, 1.6 Hz, 1H), 8.33 (m, 1H), 8.26-8.15 (m, 2H), 8.03 (ddd, J = 12.2, 7.7, 2.3 Hz, 1H), 7.88 (dd, J = 8.1, 5.1 Hz, 1H), 7.75 (m, 1H), 7.61 (ddd, J = 10.7, 8.6, 8.6 Hz, 1H), 5.53-4.42 (m, 5H), 3.37 (s, 3H). | DMSO | >98 | R3 Temper- ature at 100° C. | | | |
| 828 | 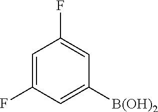 [SMILES: OB(O)c1cc(F)cc(F)c1] | 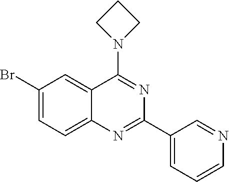 [SMILES: Brc1ccc2nc(-c3cccnc3)nc(N3CCC3)c2c1] | 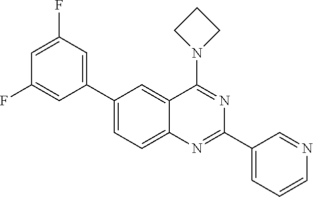 [SMILES: Fc1cc(F)cc(-c2ccc3nc(-c4cccnc4)nc(N4CCC4)c3c2)c1] | HCl | ¹H NMR (400 MHz, DMSO) δ 9.61 (d, J = 2.3 Hz, 1H), 9.05 (ddd, J = 8.0, 1.9, 1.9 Hz, 1H), 8.94 (m, 1H), 8.40-8.33 (m, 2H), 8.15 (s, 1H), 7.85 (dd, J = 8.2, 5.0 Hz, 1H), 7.68-7.58 (m, 2H), 7.33 (tt, J = 9.3, 9.3, 2.4, 2.4 Hz, 1H), 5.54-4.48 (m, 4H), 2.66-2.54 (m, 2H). | DMSO | >98 | R3 Temper- ature at 100° C. | | | |
| 829 | 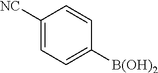 [SMILES: N#Cc1ccc(B(O)O)cc1] | 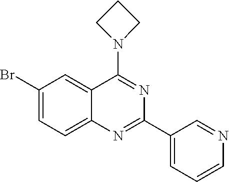 [SMILES: Brc1ccc2nc(-c3cccnc3)nc(N3CCC3)c2c1] | 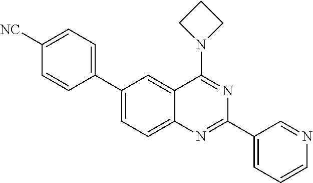 [SMILES: N#Cc1ccc(-c2ccc3nc(-c4cccnc4)nc(N4CCC4)c3c2)cc1] | 2HCl | ¹H NMR (400 MHz, DMSO) δ 9.58 (m, 1H), 9.00-8.82 (m, 2H), 8.37 (m, 1H), 8.25 (m, 1H), 8.17 (d, J = 8.8 Hz, 1H), 8.12-7.98 (m, 4H), 7.82 (m, 1H), 5.54-4.47 (m, 4H), 2.64-2.49 (m, 2H). | DMSO | >98 | R3 Temper- ature at 100° C. | | | |
| 830 | 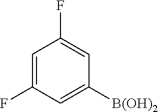 [SMILES: OB(O)c1cc(F)cc(F)c1] | 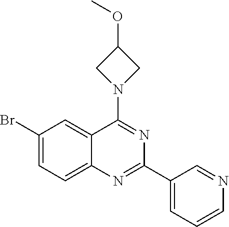 [SMILES: COC1CN(c2nc(-c3cccnc3)nc3ccc(Br)cc23)C1] | 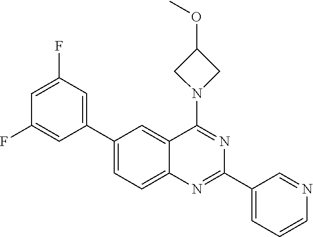 [SMILES: COC1CN(c2nc(-c3cccnc3)nc3ccc(-c4cc(F)cc(F)c4)cc23)C1] | 2HCl | ¹H NMR (400 MHz, DMSO) δ 9.59 (m, 1H), 9.01-8.88 (m, 2H), 8.34 (m, 1H), 8.22 (m, 1H), 8.10 (d, J = 8.8 Hz, 1H), 7.86 (m, 1H), 7.73-7.62 (m, 2H), 7.34 (m, 1H), 5.64-4.42 (m, 5H), 3.37 (s, 3H). | DMSO | >98 | R3 Temper- ature at 100° C. | | | |

TABLE 30

| | Starting | Starting | | | | | Salt |
|---|---|---|---|---|---|---|---|
| Number | Material 1 | Material 2 | | Product | | | type |
| 831 | 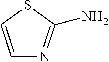 | 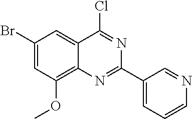 [SMILES: COc1cc(Br)cc2c(Cl)nc(-c3cccnc3)nc12] | | 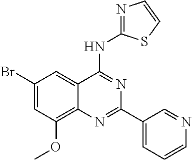 [SMILES: COc1cc(Br)cc2c(Nc3nccs3)nc(-c3cccnc3)nc12] | | | 2 HCl |
| 832 | 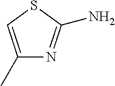 | 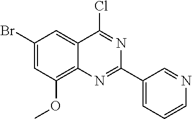 [SMILES: COc1cc(Br)cc2c(Cl)nc(-c3cccnc3)nc12] | | 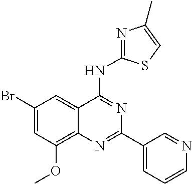 [SMILES: COc1cc(Br)cc2c(Nc3nc(C)cs3)nc(-c3cccnc3)nc12] | | | |
| 833 | 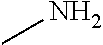 | 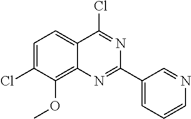 [SMILES: COc1c(Cl)ccc2c(Cl)nc(-c3cccnc3)nc12] | | 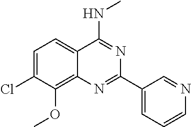 [SMILES: CNc1nc(-c2cccnc2)nc2c(OC)c(Cl)ccc12] | | | |
| 834 | 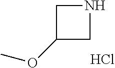 | 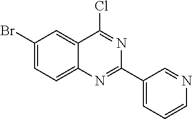 [SMILES: Clc1nc(-c2cccnc2)nc2ccc(Br)cc12] | | 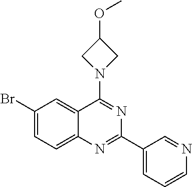 [SMILES: COC1CN(c2nc(-c3cccnc3)nc3ccc(Br)cc23)C1] | | | |
| | | ¹H NMR | Purity | Method | | Retention | LCMS |
| Number | ¹H NMR | Solvent | percent | of Coupling | LCMS | Time | Method |
| 831 | ¹H NMR (400 MHz, DMSO) δ 9.75 | DMSO | >98 | G6 | | | |
| | (d, J = 2.1 Hz, 1H), 8.87 (dt, J = | | | | | | |
| | 7.9, 1.9, 1.9 Hz, 1H), 8.74 (dd, J = | | | | | | |
| | 4.8, 1.8 Hz, 1H), 8.53 (br-s, 1H), | | | | | | |
| | 7.66-7.58 (m, 2H), 7.49 (d, J = | | | | | | |
| | 2.1 Hz, 1H), 7.35 (d, J = 3.8 Hz, | | | | | | |
| | 1H), 4.03 (s, 3H). | | | | | | |
| 832 | ¹H NMR (400 MHz, DMSO) δ 9.73 | DMSO | >98 | G6 | | | |
| | (d, J = 2.3 Hz, 1H), 8.84 (m, 1H), | | | | | | |
| | 8.71 (m, 1H), 8.41 (br-s, 1H), 7.59 | | | | | | |
| | (dd, J = 8.0, 4.8 Hz, 1H), 7.43 (m, | | | | | | |
| | 1H), 6.84 (s, 1H), 4.01 (s, 3H), 2.32 | | | | | | |
| | (s, 3H). | | | | | | |
| 833 | ¹H NMR (400 MHz, DMSO) δ 9.64 | DMSO | >98 | G4 | | | |
| | (dd, J = 2.2, 0.9 Hz, 1H), 8.78 (ddd, | | | | | | |
| | J = 7.9,1.9, 1.9 Hz, 1H), 8.70 (dd, | | | | | | |
| | J = 4.8, 1.8 Hz, 1H), 8.57 (m, 1H), | | | | | | |
| | 7.96 (d, J = 9.0 Hz, 1H), 7.57 (m, | | | | | | |
| | 1H), 4.18 (s, 3H), 3.15 (d, J = | | | | | | |
| | 4.5 Hz, 3H). | | | | | | |
| 834 | ¹H NMR (400 MHz, DMSO) δ 9.56 | DMSO | >98 | G5 | | | |
| | (dd, J = 2.2, 0.9 Hz, 1H), 8.75- | | | | | | |
| | 8.65 (m, 2H), 8.07 (d, J = 1.8 Hz, | | | | | | |
| | 1H), 7.94 (dd, J = 8.9, 2.3 Hz, 1H), | | | | | | |
| | 7.77 (d, J = 8.9 Hz, 1H), 7.54 (ddd, | | | | | | |
| | J = 7.9, 4.8, 0.9 Hz, 1H), 4.94- | | | | | | |
| | 4.69 (m, 2H), 4.57-4.33 (m, 3H), | | | | | | |
| | 3.34 (s, 3H). | | | | | | |

TABLE 31

| | Starting | Starting | | | | | Salt |
|---|---|---|---|---|---|---|---|
| Number | Material 1 | Material 2 | | Product | | | type |
| 835 | 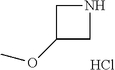 | 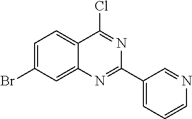 [SMILES: Clc1nc(-c2cccnc2)nc2cc(Br)ccc12] | | 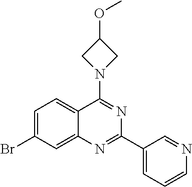 [SMILES: COC1CN(c2nc(-c3cccnc3)nc3cc(Br)ccc23)C1] | | | Free |
| 836 | 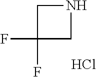 | 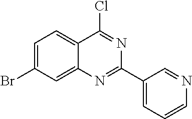 [SMILES: Clc1nc(-c2cccnc2)nc2cc(Br)ccc12] | | 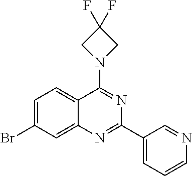 [SMILES: FC1(F)CN(c2nc(-c3cccnc3)nc3cc(Br)ccc23)C1] | | | Free |
| 837 | 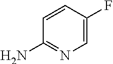 | 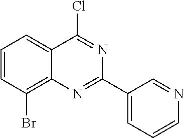 [SMILES: Clc1nc(-c2cccnc2)nc2c(Br)cccc12] | | 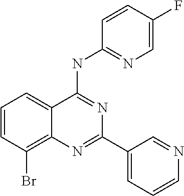 [SMILES: Fc1ccc(Nc2nc(-c3cccnc3)nc3c(Br)cccc23)nc1] | | | Free |
| | | ¹H NMR | Purity | Method | | Retention | LCMS |
| Number | ¹H NMR | Solvent | percent | of Coupling | LCMS | Time | Method |
| 835 | 1H NMR (400 MHz, CDCl3) δ 3.41 | DMSO | >98 | G5 | | | |
| | (s, 3H), 4.52-4.31 (m, 3H), 4.81- | | | | | | |
| | 4.66 (m, 2H), 7.39 (ddd, J = 8.0, | | | | | | |
| | 4.8, 0.9 Hz, 1H), 7.44 (dd, J = 8.8, | | | | | | |
| | 2.1 Hz, 1H), 7.64 (d, J = 8.8 Hz, | | | | | | |
| | 1H), 8.07 (d, J = 2.0 Hz, 1H), 8.68 | | | | | | |
| | (dd, J = 4.9, 1.7 Hz, 1H), 8.73 (dt, | | | | | | |
| | J = 8.0, 1.9 Hz, 1H), 9.68 (dd, J = | | | | | | |
| | 2.2, 0.9 Hz, 1H). | | | | | | |
| 836 | 1H NMR (400 MHz, CDCl3) δ 4.90 | DMSO | >98 | G5 | | | |
| | (t, J = 11.8 Hz, 4H), 7.41 (ddd, J = | | | | | | |
| | 7.9, 4.8, 0.9 Hz, 1H), 7.55-7.51 | | | | | | |
| | (m, 1H), 7.62-7.56 (m, 1H), 8.15 | | | | | | |
| | (dd, J = 1.8, 0.5 Hz, 1H), 8.71 (dd, | | | | | | |
| | J = 4.8, 1.7 Hz, 1H), 8.74 (dt, J = | | | | | | |
| | 8.0, 2.0 Hz, 1H), 9.68 (dd, J = 2.2, | | | | | | |
| | 0.9 Hz, 1H). | | | | | | |
| 837 | 1H NMR (400 MHz, CDCl3) δ 7.51- | DMSO | >98 | G2 | | | |
| | 7.41 (m, 2H), 7.64 (ddd, J = 9.1, | | | | | | |
| | 7.5, 3.0 Hz, 1H), 7.95 (dd, J = 8.5, | | | | | | |
| | 1.2 Hz, 1H), 8.17 (dd, J = 7.6, 1.1 Hz, | | | | | | |
| | 1H), 8.25 (d, J = 3.0 Hz, 1H), | | | | | | |
| | 8.34 (s, 1H), 8.75 (dd, J = 4.8, 1.7 Hz, | | | | | | |
| | 1H), 8.82 (ddd, J = 9.1, 4.1, 0.7 Hz, | | | | | | |
| | 1H), 8.91 (dt, J = 7.9, 2.0 Hz, | | | | | | |
| | 1H), 9.79 (dd, J = 2.2, 0.9 Hz, 1H). | | | | | | |

TABLE 32

| | | | | | | | | | | Re- | |
|---|---|---|---|---|---|---|---|---|---|---|---|
| | | | | | | | Purity | Method | | ten- | LCMS |
| Num- | Starting | Starting | | Salt | | ¹H NMR | per- | of | | tion | Meth- |
| ber | Material 1 | Material 2 | Product | type | ¹H NMR | Solvent | cent | Coupling | LCMS | Time | od |
| 838 | 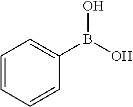 | 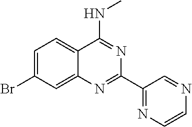 [SMILES: CNc1nc(-c2cnccn2)nc2cc(Br)ccc12] | 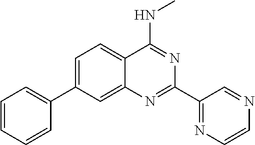 [SMILES: CNc1nc(-c2cnccn2)nc2cc(-c3ccccc3)ccc12] | HCl | 1H NMR (400 MHz, DMSO) δ 3.38 (d, J = 4.7 Hz, 3H), 7.57-7.51 (m, 1H), 7.61 (dd, J = 8.3, 6.6 Hz, 2H), 7.89-7.79 (m, 2H), 8.15 (dd, J = 8.7, 1.8 Hz, 1H), 8.52 (d, J = 1.8 Hz, 1H), 8.68 (d, J = 8.6 Hz, 1H), 9.02 (dd, J = 2.3, 1.6 Hz, 1H), 9.07 (d, J = 2.4 Hz, 1H), 9.85 (d, J = 1.4 Hz, 1H), 10.61 (s, 1H). | DMSO | >98 | R3 Temperature at 95° C. | | | |
| 839 | 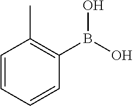 | 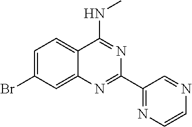 [SMILES: CNc1nc(-c2cnccn2)nc2cc(Br)ccc12] | 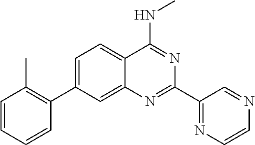 [SMILES: CNc1nc(-c2cnccn2)nc2cc(-c3ccccc3C)ccc12] | HCl | 1H NMR (400 MHz, DMSO) δ 2.32 (s, 3H),3.39 (d, J = 4.2 Hz, 3H) 7.57- 7.27 (m, 4H), 7.83 (dd, J = 8.5, 1.7 Hz, 1H), 8.22 (d, J = 1.8 Hz, 1H), 8.71 (d, J = 8.5 Hz, 1H), 9.04- 8.93 (m, 1H), 9.08 (d, J = 2.4 Hz, 1H), 9.86 (d, J = 1.5 Hz, 1H), 10.79 (s, 1H). | DMSO | >98 | R3 Temperature at 95° C. | | | |
| 840 | 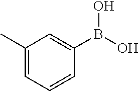 | 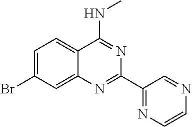 [SMILES: CNc1nc(-c2cnccn2)nc2cc(Br)ccc12] | 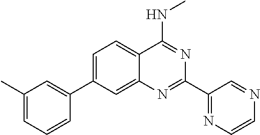 [SMILES: CNc1nc(-c2cnccn2)nc2cc(-c3cccc(C)c3)ccc12] | HCl | 1H NMR (400 MHz, DMSO) δ 2.44 (s, 3H), 3.39-3.37 (m, 3H), 7.35 (d, J = 7.5 Hz, 1H), 7.49 (t, J = 7.6 Hz, 1H), 7.64 (d, J = 8.5 Hz, 1H), 7.67 (s, 1H), 8.15 (dd, J = 8.7, 1.8 Hz, 1H), 8.52 (d, J = 1.8 Hz, 1H), 8.63 (d, J = 8.7 Hz, 1H), 9.02 (dd, J = 2.4, 1.4 Hz, 1H), 9.07 (d, J = 2.4 Hz, 1H), 9.85 (d, J = 1.4 Hz, 1H), 10.52 (s, 1H). | DMSO | >98 | R3 Temperature at 95° C. | | | |
| 841 | 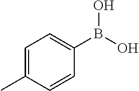 | 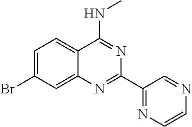 [SMILES: CNc1nc(-c2cnccn2)nc2cc(Br)ccc12] | 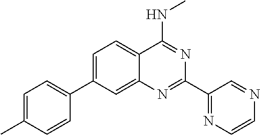 [SMILES: CNc1nc(-c2cnccn2)nc2cc(-c3ccc(C)cc3)ccc12] | HCl | 1H NMR (400 MHz, DMSO) δ 2.41 (s, 3H), 3.39-3.37 (m, 3H), 7.41 (d, J = 7.7 Hz, 2H), 7.75 (d, J = 8.1 Hz, 2H), 8.15 (dd, J = 8.6, 1.8 Hz, 1H), 8.51 (d, J = 1.9 Hz, 1H), 8.62 (d, J = 8.7 Hz, 1H), 9.05-8.99 (m, 1H), 9.08 (d, J = 2.5 Hz, 1H), 9.85 (d, J = 1.3 Hz, 1H), 10.56 (s, 1H). | DMSO | >98 | R3 Temperature at 95° C. | | | |
| 842 | 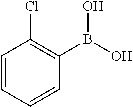 | 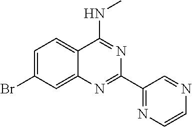 [SMILES: CNc1nc(-c2cnccn2)nc2cc(Br)ccc12] | 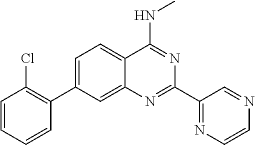 [SMILES: CNc1nc(-c2cnccn2)nc2cc(-c3ccccc3Cl)ccc12] | HCl | 1H NMR (400 MHz, DMSO) δ 3.39 (d, J = 4.6 Hz, 3H), 7.62-7.50 (m, 3H), 7.74-7.65 (m, 1H), 7.88 (dd, J = 8.6, 1.7 Hz, 1H), 8.34 (d, J = 1.7 Hz, 1H), 8.77 (d, J = 8.6 Hz, 1H), 9.01 (dd, J = 2.4, 1.4 Hz, 1H), 9.07 (d, J = 2.4 Hz, 1H), 9.85 (d, J = 1.4 Hz, 1H), 10.87 (s, 1H). | DMSO | >98 | R3 Temperature at 95° C. | | | |
| 843 | 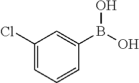 | 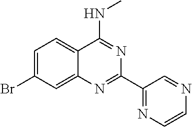 [SMILES: CNc1nc(-c2cnccn2)nc2cc(Br)ccc12] | 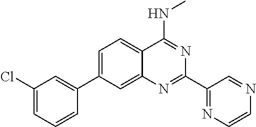 [SMILES: CNc1nc(-c2cnccn2)nc2cc(-c3cccc(Cl)c3)ccc12] | HCl | 1H NMR (400 MHz, DMSO) δ 3.37 (d, J = 4.7 Hz, 3H), 7.69-7.55 (m, 2H), 7.86-7.78 (m, 1H), 7.95- 7.88 (m, 1H), 8.18 (dd, J = 8.7, 1.8 Hz, 1H), 8.52 (d, J = 1.9 Hz, 1H), 8.64 (d, J = 8.7 Hz, 1H), 9.02-8.98 (m, 1H), 9.05 (d, J = 2.4 Hz, 1H), 9.84 (d, J = 1.4 Hz, 1H), 10.48 (s, 1H). | DMSO | >98 | R3 Temperature at 95° C. | | | |
| 844 | 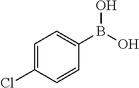 | 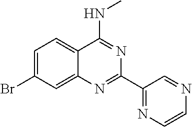 [SMILES: CNc1nc(-c2cnccn2)nc2cc(Br)ccc12] | 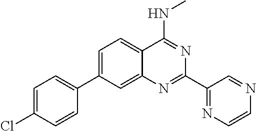 [SMILES: CNc1nc(-c2cnccn2)nc2cc(-c3ccc(Cl)cc3)ccc12] | HCl | 1H NMR (400 MHz, DMSO) δ 3.37 (d, J = 4.7 Hz, 3H), 7.68 (d, J = 7.8 Hz, 2H), 7.87 (d, J = 7.8 Hz, 2H), 8.21-8.08 (m, 1H), 8.52 (s, 1H), 8.66 (d, J = 8.5 Hz, 1H), 9.04-9.00 (m, 1H), 9.07 (d, J = 2.0 Hz, 1H), 9.85 (s, 1H), 10.60 (s, 1H). | DMSO | >98 | R3 Temperature at 95° C. | | | |
| 845 | 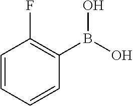 | 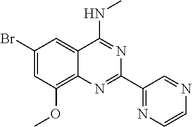 [SMILES: CNc1nc(-c2cnccn2)nc2c(OC)cc(Br)cc12] | 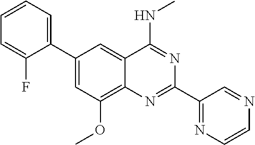 [SMILES: CNc1nc(-c2cnccn2)nc2c(OC)cc(-c3ccccc3F)cc12] | Free | 1H NMR (400 MHz, DMSO) δ 3.14 (d, J = 4.4 Hz, 3H), 4.02 (s, 3H), 7.42-7.34 (m, 2H), 7.46-7.42 (m, 1H), 7.56-7.46 (m, 1H), 7.71 (td, J = 7.8, 1.7 Hz, 1H), 8.04 - 7.97 (m, 1H), 8.57-8.48 (m, 1H), 8.74 (d, J = 2.5 Hz, 1H), 8.82 (dd, J = 2.5, 1.5 Hz, 1H), 9.64 (d, J = 1.5 Hz, 1H). | DMSO | >98 | R3 Temperature at 95° C. | | | |
| 846 | 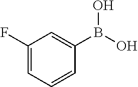 | 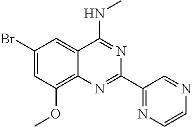 [SMILES: CNc1nc(-c2cnccn2)nc2c(OC)cc(Br)cc12] | 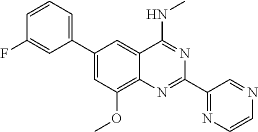 [SMILES: CNc1nc(-c2cnccn2)nc2c(OC)cc(-c3cccc(F)c3)cc12] | Free | 1H NMR (400 MHz, DMSO) δ 3.17 (d, J = 4.4 Hz, 3H), 4.08 (s, 3H), 7.31-7.22 (m, 1H), 7.62-7.52 (m, 2H), 7.83-7.74 (m, 2H), 8.19 (d, J = 1.8 Hz, 1H), 8.61-8.51 (m, 1H), 8.74 (d, J = 2.5 Hz, 1H), 8.81 (dd, J = 2.5, 1.5 Hz, 1H), 9.63 (d, J = 1.5 Hz, 1H). | DMSO | >98 | R3 Temperature at 95° C. | | | |
| 847 | 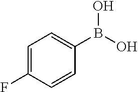 | 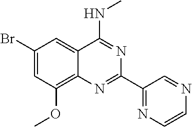 [SMILES: CNc1nc(-c2cnccn2)nc2c(OC)cc(Br)cc12] | 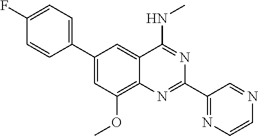 [SMILES: CNc1nc(-c2cnccn2)nc2c(OC)cc(-c3ccc(F)cc3)cc12] | Free | 1H NMR (400 MHz, DMSO) δ 3.16 (d, J = 4.4 Hz, 3H), 4.07 (s, 3H), 7.46-7.31 (m, 2H), 7.55 (d, J = 1.7 Hz, 1H), 8.00-7.89 (m, 2H), 8.12 (d, J = 1.7 Hz, 1H), 8.60-8.50 (m, 1H), 8.74 (d, J = 2.5 Hz, 1H), 8.81 (dd, J = 2.4, 1.5 Hz, 1H), 9.63 (d, J = 1.4 Hz, 1H). | DMSO | >98 | R3 Temperature at 95° C. | | | |
| 848 | 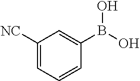 [SMILES: N#Cc1cccc(B(O)O)c1] | 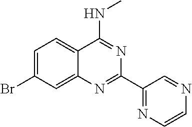 [SMILES: CNc1nc(-c2cnccn2)nc2cc(Br)ccc12] | 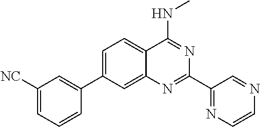 [SMILES: CNc1nc(-c2cnccn2)nc2cc(-c3cccc(C#N)c3)ccc12] | HCl | 1H NMR (400 MHz, DMSO) δ 3.38 (d, J = 4.6 Hz, 3H), 7.81 (t, J = 7.8 Hz, 1H), 8.02-7.97 (m, 1H), 8.16- 8.09 (m, 1H), 8.20 (dd, J = 8.8, 1.8 Hz, 1H), 8.32-8.26 (m, 1H), 8.54 (d, J = 1.8 Hz, 1H), 8.77 (d, J = 8.6 Hz, 1H), 9.03-8.98 (m, 1H), 9.07 (d, J = 2.4 Hz, 1H), 9.82 (d, J = 1.4 Hz, 1H), 10.92 (s, 1H). | DMSO | >98 | R3 Temperature at 95° C. | | | |
| 849 | 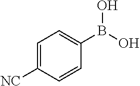 [SMILES: N#Cc1ccc(B(O)O)cc1] | 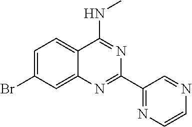 [SMILES: CNc1nc(-c2cnccn2)nc2cc(Br)ccc12] | 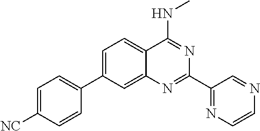 [SMILES: CNc1nc(-c2cnccn2)nc2cc(-c3ccc(C#N)cc3)ccc12] | HCl | 1H NMR (400 MHz, DMSO) δ 3.40 (d, J = 4.6 Hz, 3H), 8.07-7.96 (m, 2H), 8.12-8.08 (m, 2H), 8.30- 8.17 (m, 1H), 8.60 (s, 1H), 8.72 (d, J = 8.3 Hz, 1H), 9.04 (s, 1H), 9.09 (s, 1H), 9.87 (s, 1H), 10.77 (s, 1H). | DMSO | >98 | R3 Temperature at 95° C. | | | |
| 850 | 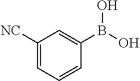 [SMILES: N#Cc1cccc(B(O)O)c1] | 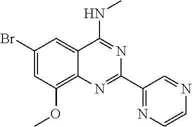 [SMILES: CNc1nc(-c2cnccn2)nc2c(OC)cc(Br)cc12] | 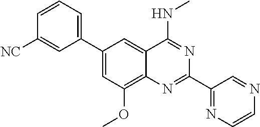 [SMILES: CNc1nc(-c2cnccn2)nc2c(OC)cc(-c3cccc(C#N)c3)cc12] | 2HCl | 1H NMR (400 MHz, DMSO) δ 3.33 (d, J = 4.4 Hz, 3H), 4.19 (s, 3H), 7.75 (t, J = 7.8 Hz, 1H), 7.97-7.89 (m, 2H), 8.37-8.31 (m, 1H), 8.49- 8.41 (m, 1H), 8.61-8.55 (m, 1H), 8.97-8.92 (m, 1H), 8.99 (d, J = 2.5 Hz, 1H), 9.70 (d, J = 1.4 Hz, 1H), 10.55 (s, 1H). | DMSO | >98 | R3 Temperature at 95° C. | | | |
| 851 | 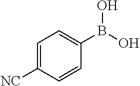 [SMILES: N#Cc1ccc(B(O)O)cc1] | 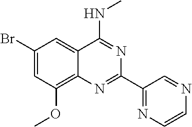 [SMILES: CNc1nc(-c2cnccn2)nc2c(OC)cc(Br)cc12] | 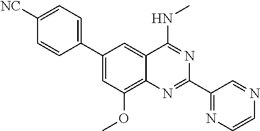 [SMILES: CNc1nc(-c2cnccn2)nc2c(OC)cc(-c3ccc(C#N)cc3)cc12] | 2HCl | 1H NMR (400 MHz, DMSO) δ 3.31 (d, J = 4.9 Hz, 3H), 4.17 (s, 3H), 7.86 (s, 1H), 8.08-8.02 (m, 2H), 8.18-8.14 (m, 2H), 8.46 (s, 1H), 8.97-8.91 (m, 2H), 9.72 (s, 1H), 9.89-10.06 (brs, 1H). | DMSO | >98 | R3 Temperature at 95° C. | | | |
| 852 | 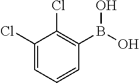 [SMILES: OB(O)c1cccc(Cl)c1Cl] | 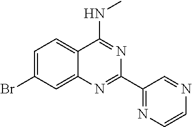 [SMILES: CNc1nc(-c2cnccn2)nc2cc(Br)ccc12] | 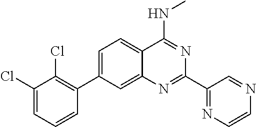 [SMILES: CNc1nc(-c2cnccn2)nc2cc(-c3cccc(Cl)c3Cl)ccc12] | Free | 1H NMR (400 MHz, DMSO) δ 3.18 (d, J = 4.3 Hz, 3H), 7.57-7.46 (m, 2H), 7.63 (dd, J = 8.5, 1.8 Hz, 1H), 7.75 (dd, J = 7.0, 2.7 Hz, 1H), 7.85 (d, J = 1.7 Hz, 1H), 8.35 (d, J = 8.5 Hz, 1H), 8.66-8.57 (m, 1H), 8.76 (d, J = 2.5 Hz, 1H), 8.82 (dd, J = 2.5, 1.5 Hz, 1H), 9.65 (d, J = 1.5 Hz, 1H). | DMSO | >98 | R3 Temperature at 95° C. | | | |
| 853 | 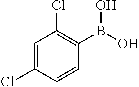 [SMILES: OB(O)c1ccc(Cl)cc1Cl] | 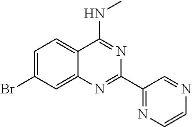 [SMILES: CNc1nc(-c2cnccn2)nc2cc(Br)ccc12] | 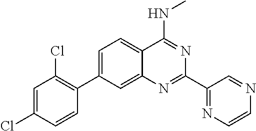 [SMILES: CNc1nc(-c2cnccn2)nc2cc(-c3ccc(Cl)cc3Cl)ccc12] | Free | 1H NMR (400 MHz, DMSO) δ 3.18 (d, J = 4.4 Hz, 3H), 7.61-7.54 (m, 2H), 7.63 (dd, J = 8.4, 1.8 Hz, 1H), 7.81 (dd, J = 1.8, 0.7 Hz, 1H), 7.86 (d, J = 1.8 Hz, 1H), 8.34 (d, J = 8.3 Hz, 1H), 8.66-8.57 (m, 1H), 8.76 (d, J = 2.5 Hz, 1H), 8.82 (dd, J = 2.5, 1.5 Hz, 1H), 9.65 (d, J = 1.5 Hz, 1H). | DMSO | >98 | R3 Temperature at 95° C. | | | |
| 854 | 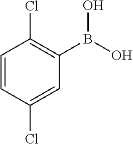 [SMILES: OB(O)c1cc(Cl)ccc1Cl] | 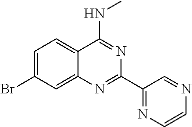 [SMILES: CNc1nc(-c2cnccn2)nc2cc(Br)ccc12] | 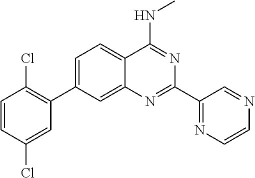 [SMILES: CNc1nc(-c2cnccn2)nc2cc(-c3cc(Cl)ccc3Cl)ccc12] | Free | 1H NMR (400 MHz, DMSO) δ 3.18 (d, J = 4.3 Hz, 3H), 7.56 (dd, J = 8.5, 2.6 Hz, 1H), 7.71-7.62 (m, 3H), 7.89 (d, J = 1.7 Hz, 1H), 8.35 (d, J = 8.5 Hz, 1H), 8.67-8.56 (m, 1H), 8.76 (d, J = 2.4 Hz, 1H), 8.82 (dd, J = 2.4, 1.5 Hz, 1H), 9.65 (d, J = 1.5 Hz, 1H). | DMSO | >98 | R3 Temperature at 95° C. | | | |
| 855 | 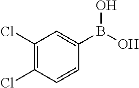 [SMILES: OB(O)c1ccc(Cl)c(Cl)c1] | 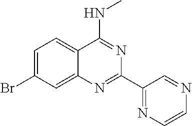 [SMILES: CNc1nc(-c2cnccn2)nc2cc(Br)ccc12] | 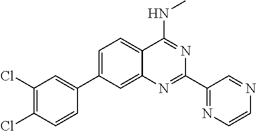 [SMILES: CNc1nc(-c2cnccn2)nc2cc(-c3ccc(Cl)c(Cl)c3)ccc12] | Free | 1H NMR (400 MHz, DMSO) δ 3.17 (d, J = 4.4 Hz, 3H), 7.77 (s, 1H), 7.92-7.88 (m, 1H), 7.95 (dd, J = 8.6, 2.0 Hz, 1H), 8.17 (t, J = 2.0 Hz, 2H), 8.35 (d, J = 8.6 Hz, 1H), 8.62- 8.56 (m, 1H), 8.75 (d, J = 2.5 Hz, 1H), 8.82 (dd, J = 2.5,1.5 Hz, 1H), 9.65 (d, J = 1.4 Hz, 1H). | DMSO | >98 | R3 Temperature at 95° C. | | | |
| 856 | 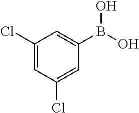 [SMILES: OB(O)c1cc(Cl)cc(Cl)c1] | 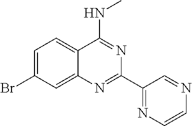 [SMILES: CNc1nc(-c2cnccn2)nc2cc(Br)ccc12] | 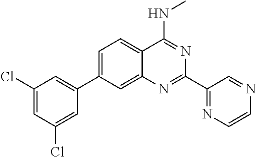 [SMILES: CNc1nc(-c2cnccn2)nc2cc(-c3cc(Cl)cc(Cl)c3)ccc12] | Free | 1H NMR (400 MHz, DMSO) δ 3.16 (d, J = 4.4 Hz, 3H), 7.69 (t, J = 1.8 Hz, 1H), 8.00-7.93 (m, 3H), 8.19 (d, J = 1.9 Hz, 1H), 8.36 (d, J = 8.6 Hz, 1H), 8.66-8.55 (m, 1H), 8.75 (d, J = 2.5 Hz, 1H), 8.82 (dd, J = 2.4, 1.5 Hz, 1H), 9.65 (d, J = 1.5 Hz, 1H). | DMSO | >98 | R3 Temperature at 95° C. | | | |
| 857 | 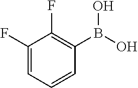 [SMILES: OB(O)c1cccc(F)c1F] | 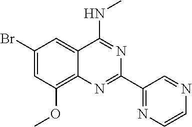 [SMILES: CNc1nc(-c2cnccn2)nc2c(OC)cc(Br)cc12] | 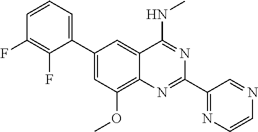 [SMILES: CNc1nc(-c2cnccn2)nc2c(OC)cc(-c3cccc(F)c3F)cc12] | Free | 1H NMR (400 MHz, DMSO) δ 3.14 (d, J = 4.4 Hz, 3H), 4.03 (s, 3H), 7.44-7.32 (m, 1H), 7.48-7.45 (m, 1H), 7.58-7.48 (m, 2H), 8.03 (s, 1H), 8.58-8.49 (m, 1H), 8.75 (d, J = 2.4 Hz, 1H), 8.82 (dd, J = 2.5, 1.5 Hz, 1H), 9.63 (d, J = 1.5 Hz, 1H). | DMSO | >98 | R3 Temperature at 95° C. | | | |
| 858 | 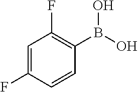 [SMILES: OB(O)c1ccc(F)cc1F] | 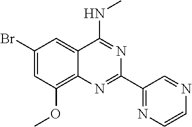 [SMILES: CNc1nc(-c2cnccn2)nc2c(OC)cc(Br)cc12] | 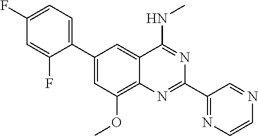 [SMILES: CNc1nc(-c2cnccn2)nc2c(OC)cc(-c3ccc(F)cc3F)cc12] | Free | 1H NMR (400 MHz, DMSO) δ 3.14 (d, J = 4.4 Hz, 3H), 4.02 (s, 3H), 7.34-7.24 (m, 1H), 7.52-7.38 (m, 2H), 7.76 (td, J = 8.9, 6.6 Hz, 1H), 8.01-7.93 (m, 1H), 8.56 - 8.46 (m, 1H), 8.74 (d, J = 2.4 Hz, 1H), 8.82 (dd, J = 2.4, 1.4 Hz, 1H), 9.63 (d, J = 1.5 Hz, 1H). | DMSO | >98 | R3 Temperature at 95° C. | | | |
| 859 | 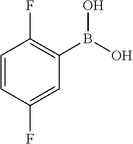 [SMILES: OB(O)c1cc(F)ccc1F] | 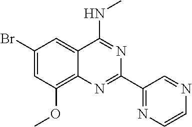 [SMILES: CNc1nc(-c2cnccn2)nc2c(OC)cc(Br)cc12] | 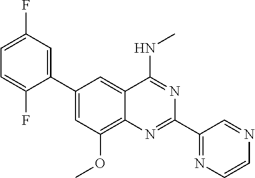 [SMILES: CNc1nc(-c2cnccn2)nc2c(OC)cc(-c3cc(F)ccc3F)cc12] | Free | 1H NMR (400 MHz, DMSO) δ 3.15 (d, J = 4.4 Hz, 3H), 4.03 (s, 3H), 7.38-7.30 (m, 1H), 7.53-7.40 (m, 2H), 7.61 (ddd, J = 9.3, 6.1, 3.2 Hz, 1H), 8.03 (s, 1H), 8.58-8.47 (m, 1H), 8.74 (d, J = 2.5 Hz, 1H), 8.82 (dd, J = 2.5, 1.5 Hz, 1H), 9.63 (d, J = 1.5 Hz, 1H). | DMSO | >98 | R3 Temperature at 95° C. | | | |
| 860 | 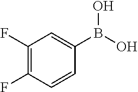 [SMILES: OB(O)c1ccc(F)c(F)c1] | 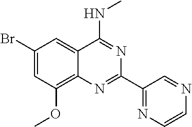 [SMILES: CNc1nc(-c2cnccn2)nc2c(OC)cc(Br)cc12] | 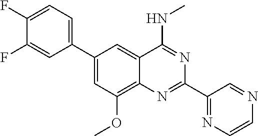 [SMILES: CNc1nc(-c2cnccn2)nc2c(OC)cc(-c3ccc(F)c(F)c3)cc12] | Free | 1H NMR (400 MHz, DMSO) δ 3.16 (d, J = 4.4 Hz, 3H), 4.07 (s, 3H), 7.69-7.50 (m, 2H), 7.82-7.74 (m, 1H), 8.01 (ddd, J = 12.4, 7.8, 2.3 Hz, 1H), 8.14 (d, J = 1.7 Hz, 1H), 8.55-8.47 (m, 1H), 8.73 (d, J = 2.4 Hz, 1H), 8.81 (dd, J = 2.4, 1.5 Hz, 1H), 9.62 (d, J = 1.5 Hz, 1H). | DMSO | >98 | R3 Temperature at 95° C. | | | |
| 861 | 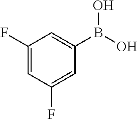 [SMILES: OB(O)c1cc(F)cc(F)c1] | 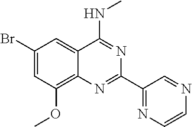 [SMILES: CNc1nc(-c2cnccn2)nc2c(OC)cc(Br)cc12] | 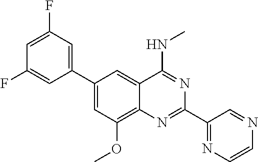 [SMILES: CNc1nc(-c2cnccn2)nc2c(OC)cc(-c3cc(F)cc(F)c3)cc12] | Free | 1H NMR (400 MHz, DMSO) δ 3.16 (d, J = 4.4 Hz, 3H), 4.08 (s, 3H), 7.35-7.22 (m, 1H), 7.60 (d, J = 1.8 Hz, 1H), 7.74-7.63 (m, 2H), 8.18 (d, J = 1.8 Hz, 1H), 8.57-8.45 (m, 1H), 8.73 (d, J = 2.5 Hz, 1H), 8.81 (dd, J = 2.5, 1.5 Hz, 1H), 9.61 (d, J = 1.5 Hz, 1H). | DMSO | >98 | R3 Temperature at 95° C. | | | |
| 862 | 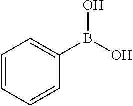 | 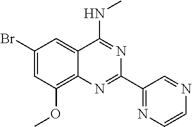 [SMILES: CNc1nc(-c2cnccn2)nc2c(OC)cc(Br)cc12] | 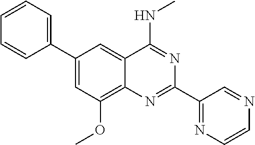 [SMILES: CNc1nc(-c2cnccn2)nc2c(OC)cc(-c3ccccc3)cc12] | Free | 1H NMR (400 MHz, DMSO) δ 3.16 (d, J = 4.4 Hz, 3H), 4.07 (s, 3H), 7.49-7.40 (m, 1H), 7.60-7.50 (m, 3H), 7.99-7.88 (m, 2H), 8.15 (d, J = 1.8 Hz, 1H), 8.61-8.51 (m, 1H), 8.74 (d, J = 2.5 Hz, 1H), 8.81 (dd, J = 2.5, 1.5 Hz, 1H), 9.63 (d, J = 1.5 Hz, 1H). | DMSO | >98 | R3 Temperature at 95° C. | | | |
| 863 | 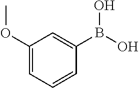 | 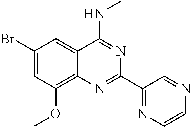 [SMILES: CNc1nc(-c2cnccn2)nc2c(OC)cc(Br)cc12] | 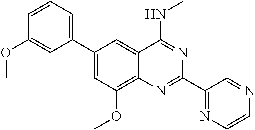 [SMILES: CNc1nc(-c2cnccn2)nc2c(OC)cc(-c3cccc(OC)c3)cc12] | Free | 1H NMR (400 MHz, DMSO) δ 3.16 (d, J = 4.4 Hz, 3H), 3.33 (s, 3H), 4.08 (s, 3H), 7.08-6.98 (m, 1H), 7.52-7.42 (m, 3H), 7.56 (d, J = 1.6 Hz, 1H), 8.12 (d, J = 1.7 Hz, 1H), 8.56-8.46 (m, 1H), 8.73 (d, J = 2.5 Hz, 1H), 8.81 (dd, J = 2.5, 1.5 Hz, 1H), 9.63 (d, 3 = 1.5 Hz, 1H). | DMSO | >98 | R3 Temperature at 95° C. | | | |
| 864 | 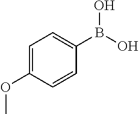 | 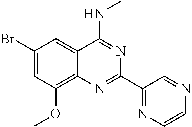 [SMILES: CNc1nc(-c2cnccn2)nc2c(OC)cc(Br)cc12] | 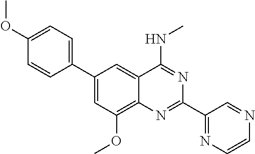 [SMILES: CNc1nc(-c2cnccn2)nc2c(OC)cc(-c3ccc(OC)cc3)cc12] | HCl | 1H NMR (400 MHz, DMSO) δ 3.30- 3.40 (m, 3H), 3.85 (s, 3H), 4.18 (s, 3H), 7.17-7.08 (m, 2H), 7.80 (s, 1H), 7.97-7.86 (m, 2H), 8.30 (s, 1H), 8.96 (d, J = 9.8 Hz, 2H), 9.73 (s, 1H), 9.70-10.22 (brs, 1H). | DMSO | >98 | R3 Temperature at 95° C. | | | |
| 865 | 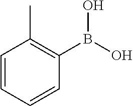 | 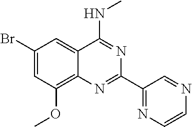 [SMILES: CNc1nc(-c2cnccn2)nc2c(OC)cc(Br)cc12] | 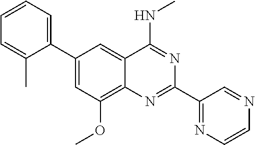 [SMILES: CNc1nc(-c2cnccn2)nc2c(OC)cc(-c3ccccc3C)cc12] | Free | 1H NMR (400 MHz, DMSO) δ 2.33 (s, 3H), 3.12 (d, J = 4.4 Hz, 3H), 4.00 (s, 3H), 7.26 (d, J = 1.5 Hz, 1H), 7.40-7.30 (m, 4H), 7.77 (d, J = 1.6 Hz, 1H), 8.39 (q, J = 4.4 Hz, 1H), 8.74 (d, J = 2.5 Hz, 1H), 8.82 (dd, J = 2.5, 1.5 Hz, 1H), 9.62 (d, J = 1.5 Hz, 1H). | DMSO | >98 | R3 Temperature at 95° C. | | | |
| 866 | 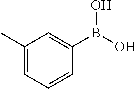 | 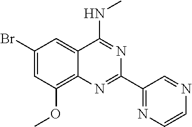 [SMILES: CNc1nc(-c2cnccn2)nc2c(OC)cc(Br)cc12] | 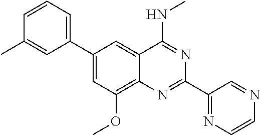 [SMILES: CNc1nc(-c2cnccn2)nc2c(OC)cc(-c3cccc(C)c3)cc12] | Free | 1H NMR (400 MHz, DMSO) δ 2.44 (s, 3H), 3.16 (d, J = 4.4 Hz, 3H), 4.07 (s, 3H), 7.28-7.20 (m, 1H), 7.43 (t, J = 7.6 Hz, 1H), 7.56 (d, J = 1.6 Hz, 1H), 7.69 (d, J = 8.0 Hz, 1H), 7.72 (s, 1H), 8.12 (d, J = 1.7 Hz, 1H), 8.60-8.48 (m, 1H), 8.74 (d, J = 2.4 Hz, 1H), 8.81 (dd, J = 2.5, 1.5 Hz, 1H), 9.63 (d, J = 1.5 Hz, 1H). | DMSO | >98 | R3 Temperature at 95° C. | | | |
| 867 | 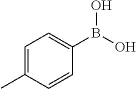 | 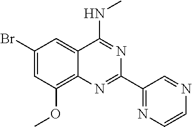 [SMILES: CNc1nc(-c2cnccn2)nc2c(OC)cc(Br)cc12] | 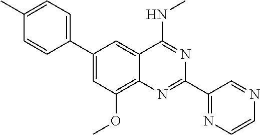 [SMILES: CNc1nc(-c2cnccn2)nc2c(OC)cc(-c3ccc(C)cc3)cc12] | Free | 1H NMR (400 MHz, DMSO) δ 2.39 (s, 3H), 3.16 (d, J = 4.4 Hz, 3H), 4.06 (s, 3H), 7.35 (d, J = 7.8 Hz, 2H), 7.55 (d, J = 1.7 Hz, 1H), 7.84 - 7.76 (m, 2H), 8.12 (d, J = 1.8 Hz, 1H), 8.59-8.50 (m, 1H), 8.73 (d, J = 2.5 Hz, 1H), 8.80 (dd, J = 2.4, 1.4 Hz, 1H), 9.63 (d, J = 1.4 Hz, 1H). | DMSO | >98 | R3 Temperature at 95° C. | | | |
| 868 | 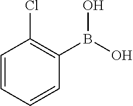 | 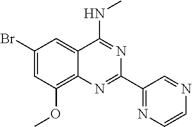 [SMILES: CNc1nc(-c2cnccn2)nc2c(OC)cc(Br)cc12] | 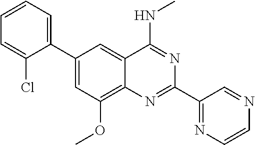 [SMILES: CNc1nc(-c2cnccn2)nc2c(OC)cc(-c3ccccc3Cl)cc12] | Free | 1H NMR (400 MHz, DMSO) δ 3.13 (d, J = 4.3 Hz, 3H), 4.00 (s, 3H), 7.35 (d, J = 1.6 Hz, 1H), 7.53-7.45 (m, 2H), 7.58 (dd, J = 7.3, 2.1 Hz, 1H), 7.64 (dd, J = 7.4, 1.9 Hz, 1H), 7.87 (d, J = 1.7 Hz, 1H), 8.50-8.39 (m, 1H), 8.75 (d, J = 2.4 Hz, 1H), 8.84-8.80 (m, 1H), 9.64 (d, J = 1.5 Hz, 1H). | DMSO | >98 | R3 Temperature at 95° C. | | | |
| 869 | 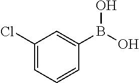 | 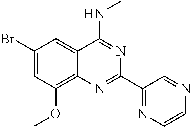 [SMILES: CNc1nc(-c2cnccn2)nc2c(OC)cc(Br)cc12] | 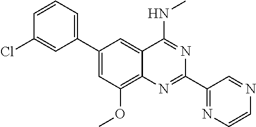 [SMILES: CNc1nc(-c2cnccn2)nc2c(OC)cc(-c3cccc(Cl)c3)cc12] | Free | 1H NMR (400 MHz, DMSO) δ 3.17 (d, J = 4.4 Hz, 3H), 4.08 (s, 3H), 7.51-7.47 (m, 1H), 7.60-7.54 (m, 2H), 7.93-7.86 (m, 1H), 7.99 (t, J = 1.9 Hz, 1H), 8.16 (d, J = 1.8 Hz, 1H), 8.59-8.52 (m, 1H), 8.74 (d, J = 2.5 Hz, 1H), 8.81 (dd, J = 2.4, 1.4 Hz, 1H), 9.63 (d, J = 1.5 Hz, 1H). | DMSO | >98 | R3 Temperature at 95° C. | | | |
| 870 | 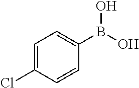 | 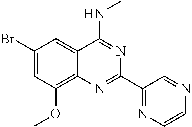 [SMILES: CNc1nc(-c2cnccn2)nc2c(OC)cc(Br)cc12] | 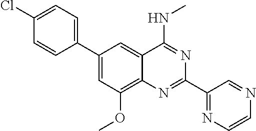 [SMILES: CNc1nc(-c2cnccn2)nc2c(OC)cc(-c3ccc(Cl)cc3)cc12] | Free | 1H NMR (400 MHz, DMSO) δ 3.16 (d, J = 4.4 Hz, 3H), 4.07 (s, 3H), 7.57 (d, J = 1.7 Hz, 1H), 7.64-7.59 (m, 2H), 7.99-7.90 (m, 2H), 8.15 (d, J = 1.8 Hz, 1H), 8.58-8.52 (m, 1H), 8.74 (d, J = 2.4 Hz, 1H), 8.81 (dd, J = 2.5, 1.5 Hz, 1H), 9.62 (d, J = 1.5 Hz, 1H). | DMSO | >98 | R3 Temperature at 95° C. | | | |
| 871 | 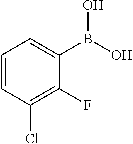 [SMILES: OB(O)c1cccc(Cl)c1F] | 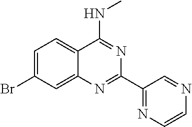 [SMILES: CNc1nc(-c2cnccn2)nc2cc(Br)ccc12] | 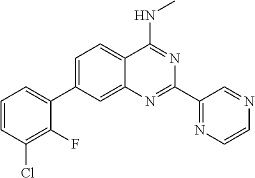 [SMILES: CNc1nc(-c2cnccn2)nc2cc(-c3cccc(Cl)c3F)ccc12] | Free | 1H NMR (400 MHz, DMSO) δ 3.18 (d, J = 4.4 Hz, 3H), 7.40 (td, J = 7.9, 1.0 Hz, 1H), 7.70 (dd, J = 8.0, 7.0 Hz, 2H), 7.77 (dt, J = 8.6, 1.8 Hz, 1H), 8.00 (t, J = 1.6 Hz, 1H), 8.37 (d, J = 8.6 Hz, 1H), 8.68-8.59 (m, 1H), 8.76 (d, J = 2.6 Hz, 1H), 8.82 (dd, J = 2.6, 1.5 Hz, 1H), 9.65 (d, J = 1.5 Hz, 1H). | DMSO | >98 | R4 Temperature at 80° C. | | | |
| 872 | 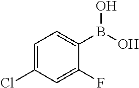 [SMILES: OB(O)c1ccc(Cl)cc1F] | 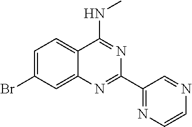 [SMILES: CNc1nc(-c2cnccn2)nc2cc(Br)ccc12] | 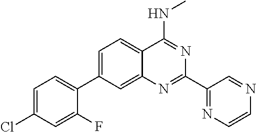 [SMILES: CNc1nc(-c2cnccn2)nc2cc(-c3ccc(Cl)cc3F)ccc12] | Free | 1H NMR (400 MHz, DMSO) δ 3.17 (d, J = 4.3 Hz, 3H), 7.51-7.42 (m, 1H), 7.64 (dd, J = 10.7, 2.1 Hz, 1H), 7.80-7.70 (m, 2H), 7.99 (t, J = 1.7 Hz, 1H), 8.36 (d, J = 8.6 Hz, 1H), 8.66-8.56 (m, 1H), 8.76 (d, J = 2.5 Hz, 1H), 8.82 (dd, J = 2.5, 1.5 Hz, 1H), 9.65 (d, J = 1.5 Hz, 1H). | DMSO | >98 | R4 Temperature at 80° C. | | | |
| 873 | 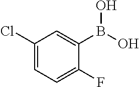 [SMILES: OB(O)c1cc(Cl)ccc1F] | 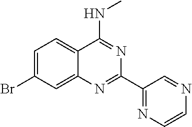 [SMILES: CNc1nc(-c2cnccn2)nc2cc(Br)ccc12] | 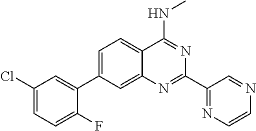 [SMILES: CNc1nc(-c2cnccn2)nc2cc(-c3cc(Cl)ccc3F)ccc12] | Free | 1H NMR (400 MHz, DMSO) δ 3.17 (d, J = 4.3 Hz, 3H), 7.47 (dd, J = 10.4, 8.8 Hz, 1H), 7.57 (ddd, J = 8.8, 4.3, 2.7 Hz, 1H), 7.86-7.72 (m, 2H), 8.05-7.98 (m, 1H), 8.36 (d, J = 8.6 Hz, 1H), 8.68-8.56 (m, 1H), 8.76 (d, J = 2.4 Hz, 1H), 8.82 (dd, J = 2.5, 1.5 Hz, 1H), 9.65 (d, J = 1.5 Hz, 1H). | DMSO | >98 | R4 Temperature at 80° C. | | | |
| 874 | 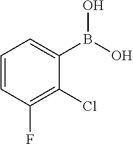 [SMILES: OB(O)c1cccc(F)c1Cl] | 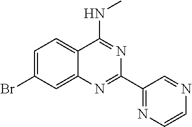 [SMILES: CNc1nc(-c2cnccn2)nc2cc(Br)ccc12] | 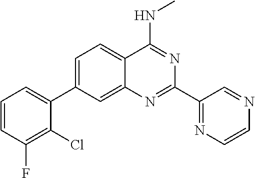 [SMILES: CNc1nc(-c2cnccn2)nc2cc(-c3cccc(F)c3Cl)ccc12] | Free | 1H NMR (400 MHz, DMSO) δ 3.18 (s, 3H), 7.47-7.39 (m, 1H), 7.59- 7.50 (m, 2H), 7.65 (dd, J = 8.4, 1.8 Hz, 1H), 7.88 (d, J = 1.7 Hz, 1H), 8.37 (d, J = 8.5 Hz, 1H), 8.65 (brs, 1H), 8.76 (d, J = 2.5 Hz, 1H), 8.82 (dd, J = 2.4, 1.5 Hz, 1H), 9.65 (d, J = 1.4 Hz, 1H). | DMSO | >98 | R4 Temperature at 80° C. | | | |
| 875 | 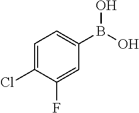 [SMILES: OB(O)c1ccc(Cl)c(F)c1] | 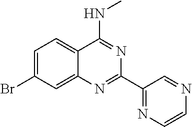 [SMILES: CNc1nc(-c2cnccn2)nc2cc(Br)ccc12] | 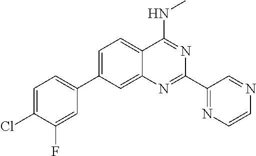 [SMILES: CNc1nc(-c2cnccn2)nc2cc(-c3ccc(Cl)c(F)c3)ccc12] | Free | 1H NMR (400 MHz, DMSO) δ 3.17 (d, J = 4.4 Hz, 3H), 7.76-7.64 (m, 1H), 7.83-7.76 (m, 1H), 7.95 (dd, J = 8.6, 1.9 Hz, 1H), 8.00 (dd, J = 10.9, 2.0 Hz, 1H), 8.17 (d, J = 1.9 Hz, 1H), 8.35 (d, J = 8.6 Hz, 1H), 8.64-8.56 (m, 1H), 8.76 (d, J = 2.5 Hz, 1H), 8.82 (dd, J = 2.4, 1.5 Hz, 1H), 9.65 (d, J = 1.5 Hz, 1H). | DMSO | >98 | R4 Temperature at 80° C. | | | |
| 876 | 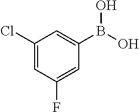 [SMILES: OB(O)c1cc(F)cc(Cl)c1] | 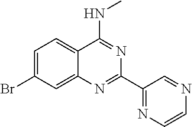 [SMILES: CNc1nc(-c2cnccn2)nc2cc(Br)ccc12] | 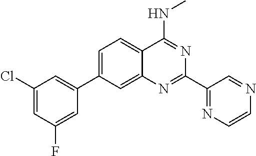 [SMILES: CNc1nc(-c2cnccn2)nc2cc(-c3cc(F)cc(Cl)c3)ccc12] | Free | 1H NMR (400 MHz, DMSO) δ 3.16 (d, J = 4.4 Hz, 3H), 7.52 (dt, J = 8.6, 2.1 Hz, 1H), 7.80 (dt, J = 10.1, 2.0 Hz, 1H), 7.86-7.83 (m, 1H), 7.97 (dd, J = 8.7, 2.0 Hz, 1H), 8.19 (d, J = 1.9 Hz, 1H), 8.36 (d, J = 8.6 Hz, 1H), 8.67-8.61 (m, 1H), 8.76 (d, J = 2.5 Hz, 1H), 8.82 (dd, J = 2.4, 1.5 Hz, 1H), 9.65 (d, J = 1.5 Hz, 1H). | DMSO | >98 | R4 Temperature at 80° C. | | | |
| 877 | 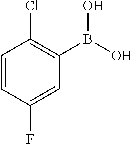 [SMILES: OB(O)c1cc(F)ccc1Cl] | 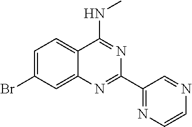 [SMILES: CNc1nc(-c2cnccn2)nc2cc(Br)ccc12] | 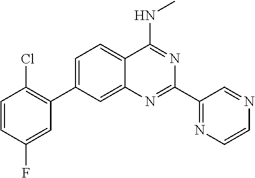 [SMILES: CNc1nc(-c2cnccn2)nc2cc(-c3cc(F)ccc3Cl)ccc12] | Free | 1H NMR (400 MHz, DMSO) δ 3.18 (d, J = 4.3 Hz, 3H), 7.37 (ddd, J = 8.9, 8.1, 3.1 Hz, 1H), 7.50 (dd, J = 9.2, 3.1 Hz, 1H), 7.65 (dd, J = 8.4, 1.8 Hz, 1H), 7.69 (dd, J = 8.9, 5.2 Hz, 1H), 7.89 (d, J = 1.7 Hz, 1H), 8.35 (d, J = 8.5 Hz, 1H), 8.67-8.58 (m, 1H), 8.76 (d, J = 2.4 Hz, 1H), 8.82 (dd, J = 2.5, 1.5 Hz, 1H), 9.65 (d, J = 1.5 Hz, 1H). | DMSO | >98 | R4 Temperature at 80° C. | | | |
| 878 | 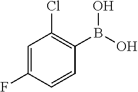 [SMILES: OB(O)c1ccc(F)cc1Cl] | 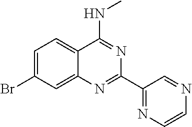 [SMILES: CNc1nc(-c2cnccn2)nc2cc(Br)ccc12] | 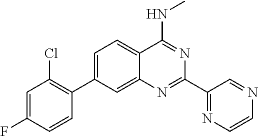 [SMILES: CNc1nc(-c2cnccn2)nc2cc(-c3ccc(F)cc3Cl)ccc12] | Free | 1H NMR (400 MHz, DMSO) δ 3.18 (d, J = 4.4 Hz, 3H), 7.39 (td, J = 8.5, 2.6 Hz, 1H), 7.69-7.57 (m, 3H), 7.85 (d, J = 1.7 Hz, 1H), 8.34 (d, J = 8.4 Hz, 1H), 8.68-8.55 (m, 1H), 8.76 (d, J = 2.5 Hz, 1H), 8.82 (dd, J = 2.5, 1.5 Hz, 1H), 9.65 (d, J = 1.5 Hz, 1H). | DMSO | >98 | R4 Temperature at 80° C. | | | |
| 879 | 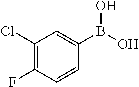 [SMILES: OB(O)c1ccc(F)c(Cl)c1] | 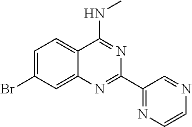 [SMILES: CNc1nc(-c2cnccn2)nc2cc(Br)ccc12] | 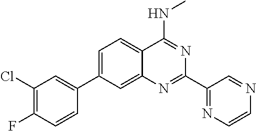 [SMILES: CNc1nc(-c2cnccn2)nc2cc(-c3ccc(F)c(Cl)c3)ccc12] | Free | 1H NMR (400 MHz, DMSO) δ 3.16 (d, J = 4.3 Hz, 3H), 7.57 (t, J = 8.9 Hz, 1H), 7.98-7.88 (m, 2H), 8.18- 8.09 (m, 2H), 8.34 (d, J = 8.6 Hz, 1H), 8.62-8.53 (m, 1H), 8.75 (d, J = 2.5 Hz, 1H), 8.82 (dd, J = 2.5, 1.5 Hz, 1H), 9.65 (d, J = 1.5 Hz, 1H). | DMSO | >98 | R4 Temperature at 80° C. | | | |
| 880 | 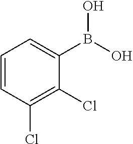 [SMILES: OB(O)c1cccc(Cl)c1Cl] | 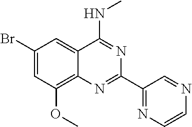 [SMILES: CNc1nc(-c2cnccn2)nc2c(OC)cc(Br)cc12] | 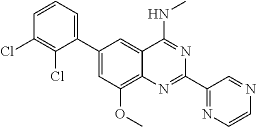 [SMILES: CNc1nc(-c2cnccn2)nc2c(OC)cc(-c3cccc(Cl)c3Cl)cc12] | Free | 1H NMR (400 MHz, DMSO) δ 3.12 (d, J = 4.4 Hz, 3H), 4.00 (s, 3H), 7.37 (d, J = 1.6 Hz, 1H), 7.58-7.47 (m, 2H), 7.74 (dd, J = 7.5, 2.1 Hz, 1H), 7.87 (d, J = 1.7 Hz, 1H), 8.48- 8.41 (m, 1H), 8.75 (d, J = 2.5 Hz, 1H), 8.82 (dd, J = 2.5, 1.5 Hz, 1H), 9.63 (d, J = 1.5 Hz, 1H). | DMSO | >98 | R4 Temperature at 80° C. | | | |
| 881 | 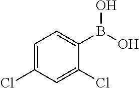 [SMILES: OB(O)c1ccc(Cl)cc1Cl] | 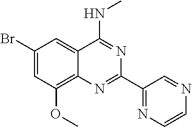 [SMILES: CNc1nc(-c2cnccn2)nc2c(OC)cc(Br)cc12] | 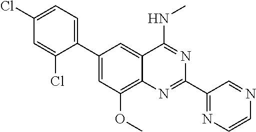 [SMILES: CNc1nc(-c2cnccn2)nc2c(OC)cc(-c3ccc(Cl)cc3Cl)cc12] | Free | 1H NMR (400 MHz, DMSO) δ 3.13 (d, J = 4.3 Hz, 3H), 4.00 (s, 3H), 7.34 (d, J = 1.6 Hz, 1H), 7.60 (d, J = 1.0 Hz, 2H), 7.84-7.79 (m, 1H), 7.88 (d, J = 1.7 Hz, 1H), 8.55-8.48 (m, 1H), 8.74 (d, J = 2.5 Hz, 1H), 8.82 (dd, J = 2.4, 1.4 Hz, 1H), 9.63 (d, J = 1.5 Hz, 1H). | DMSO | >98 | R4 Temperature at 80° C. | | | |
| 882 | 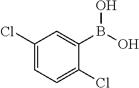 [SMILES: OB(O)c1cc(Cl)ccc1Cl] | 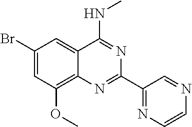 [SMILES: CNc1nc(-c2cnccn2)nc2c(OC)cc(Br)cc12] | 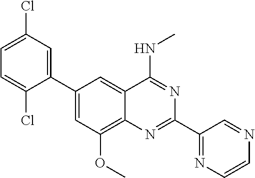 [SMILES: CNc1nc(-c2cnccn2)nc2c(OC)cc(-c3cc(Cl)ccc3Cl)cc12] | Free | 1H NMR (400 MHz, DMSO) δ 3.13 (d, J = 4.4 Hz, 3H), 4.01 (s, 3H), 7.38 (d, J = 1.6 Hz, 1H), 7.58-7.53 (m, 1H), 7.72-7.65 (m, 2H), 7.90 (d, J = 1.7 Hz, 1H), 8.57-8.44 (m, 1H), 8.75 (d, J = 2.5 Hz, 1H), 8.82 (dd, J = 2.4, 1.4 Hz, 1H), 9.64 (d, J = 1.5 Hz, 1H). | DMSO | >98 | R4 Temperature at 80° C. | | | |
| 883 | 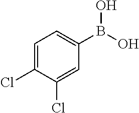 [SMILES: OB(O)c1ccc(Cl)c(Cl)c1] | 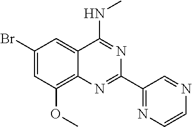 [SMILES: CNc1nc(-c2cnccn2)nc2c(OC)cc(Br)cc12] | 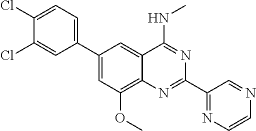 [SMILES: CNc1nc(-c2cnccn2)nc2c(OC)cc(-c3ccc(Cl)c(Cl)c3)cc12] | Free | 1H NMR (400 MHz, DMSO) δ 3.17 (d, J = 4.4 Hz, 3H), 4.08 (s, 3H), 7.60 (d, J = 1.7 Hz, 1H), 7.80 (d, J = 8.5 Hz, 1H), 7.92 (dd, J = 8.5, 2.2 Hz, 1H), 8.18 (dd, J = 5.9, 2.0 Hz, 2H), 8.62-8.54 (m, 1H), 8.74 (d, J = 2.5 Hz, 1H), 8.81 (dd, J = 2.5, 1.5 Hz, 1H), 9.62 (d, J = 1.5 Hz, 1H). | DMSO | >98 | R4 Temperature at 80° C. | | | |
| 884 | 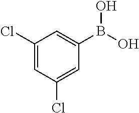 [SMILES: OB(O)c1cc(Cl)cc(Cl)c1] | 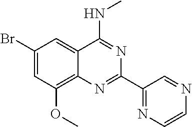 [SMILES: CNc1nc(-c2cnccn2)nc2c(OC)cc(Br)cc12] | 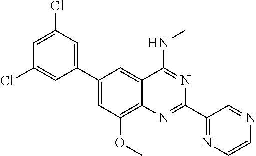 [SMILES: CNc1nc(-c2cnccn2)nc2c(OC)cc(-c3cc(Cl)cc(Cl)c3)cc12] | Free | 1H NMR (400 MHz, DMSO) δ 3.16 (d, J = 4.4 Hz, 3H), 4.08 (s, 3H), 7.60 (d, J = 1.7 Hz, 1H), 7.65 (t, J = 1.8 Hz, 1H), 7.98 (d, J = 1.9 Hz, 2H), 8.17 (d, J = 1.8 Hz, 1H), 8.60- 8.54 (m, 1H), 8.73 (d, J = 2.5 Hz, 1H), 8.81 (dd, J = 2.4, 1.5 Hz, 1H), 9.61 (d, J = 1.5 Hz, 1H). | DMSO | >98 | R4 Temperature at 80° C. | | | |
| 885 | 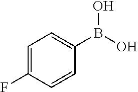 | 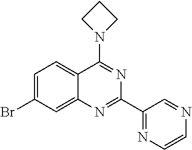 [SMILES: Brc1ccc2c(N3CCC3)nc(-c3cnccn3)nc2c1] | 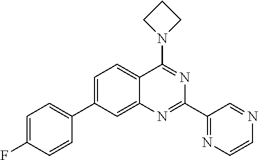 [SMILES: Fc1ccc(-c2ccc3c(N4CCC4)nc(-c4cnccn4)nc3c2)cc1] | HCl | 1H NMR (400 MHz, DMSO) δ 2.67- 2.54 (m, 2H), 4.72 (brs, 2H), 5.09 (brs, 2H), 7.52-7.41 (m, 2H), 7.91- 7.85 (m, 2H), 8.03 (dd, J = 8.7, 1.9 Hz, 1H), 8.18 (d, J = 8.7 Hz, 1H), 8.52 (d, J = 1.8 Hz, 1H), 9.00 (dd, J = 2.4, 1.5 Hz, 1H), 9.06 (d, J = 2.5 Hz, 1H), 9.72 (d, J = 1.4 Hz, 1H). | DMSO | >98 | R4 Temperature at 80° C. | | | |
| 886 | 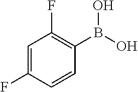 [SMILES: OB(O)c1ccc(F)cc1F] | 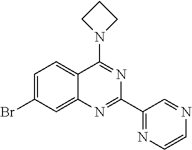 [SMILES: Brc1ccc2c(N3CCC3)nc(-c3cnccn3)nc2c1] | 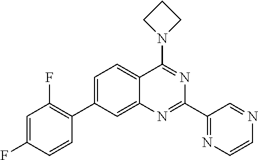 [SMILES: Fc1ccc(-c2ccc3c(N4CCC4)nc(-c4cnccn4)nc3c2)c(F)c1] | HCl | 1H NMR (400 MHz, DMSO) δ 2.66- 2.55 (m, 2H), 4.72 (brs, 2H), 5.09 (brs, 2H), 7.40-7.30 (m, 1H), 7.53 (ddd, J = 11.6, 9.3, 2.6 Hz, 1H), 7.77 (td, J = 8.9, 6.4 Hz, 1H), 7.95- 7.85 (m, 1H), 8.22 (d, J = 8.7 Hz, 1H), 8.48-8.42 (m, 1H), 8.99 (dd, J = 2.5, 1.5 Hz, 1H), 9.05 (d, J = 2.5 Hz, 1H), 9.72 (d, J = 1.4 Hz, 1H). | DMSO | >98 | R4 Temperature at 80° C. | | | |
| 887 | 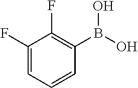 [SMILES: OB(O)c1cccc(F)c1F] | 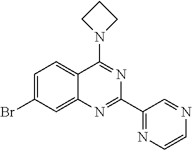 [SMILES: Brc1ccc2c(N3CCC3)nc(-c3cnccn3)nc2c1] | 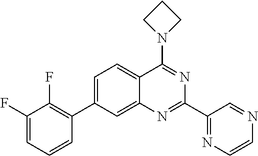 [SMILES: Fc1cccc(-c2ccc3c(N4CCC4)nc(-c4cnccn4)nc3c2)c1F] | HCl | 1H NMR (400 MHz, DMSO) δ 2.68 - 2.56 (m, 2H), 4.73 (brs, 2H), 5.10 (brs, 2H), 7.49-7.41 (m, 1H), 7.55- 7.49 (m, 1H), 7.68-7.57 (m, 1H), 7.93 (dt, J = 8.7, 1.6 Hz, 1H), 8.24 (d, J = 8.7 Hz, 1H), 8.51-8.44 (m, 1H), 9.00 (dd, J = 2.5, 1.5 Hz, 1H), 9.06 (d, J = 2.4 Hz, 1H), 9.73 (d, J = 1.4 Hz, 1H). | DMSO | >98 | R4 Temperature at 80° C. | | | |
| 888 | 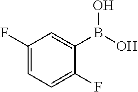 [SMILES: OB(O)c1cc(F)ccc1F] | 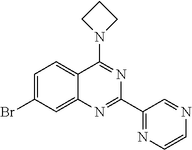 [SMILES: Brc1ccc2c(N3CCC3)nc(-c3cnccn3)nc2c1] | 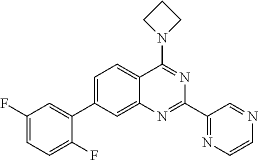 [SMILES: Fc1ccc(F)c(-c2ccc3c(N4CCC4)nc(-c4cnccn4)nc3c2)c1] | HCl | 1H NMR (400 MHz, DMSO) δ 2.67- 2.56 (m, 2H), 4.73 (brs, 2H), 5.08 (brs, 2H), 7.49-7.41 (m, 1H), 7.53 (ddd, J = 10.2,9.1, 4.6 Hz, 1H), 7.61 (ddd, J = 9.2, 6.1, 3.2 Hz, 1H), 7.93 (dt, J = 8.7, 1.5 Hz, 1H), 8.22 (d, J = 8.7 Hz, 1H), 8.50-8.44 (m, 1H), 8.99 (dd, J = 2.5, 1.5 Hz, 1H), 9.05 (d, J = 2.4 Hz, 1H), 9.73 (d, J = 1.4 Hz, 1H). | DMSO | >98 | R4 Temperature at 80° C. | | | |
| 889 | 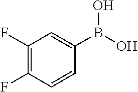 [SMILES: OB(O)c1ccc(F)c(F)c1] | 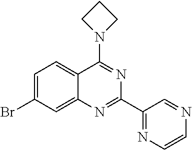 [SMILES: Brc1ccc2c(N3CCC3)nc(-c3cnccn3)nc2c1] | 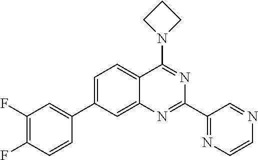 [SMILES: Fc1ccc(-c2ccc3c(N4CCC4)nc(-c4cnccn4)nc3c2)cc1F] | HCl | 1H NMR (400 MHz, DMSO) δ 2.66- 2.55 (m, 2H), 4.71 (brs, 2H), 5.08 (brs, 2H), 7.75-7.61 (m, 2H), 7.99- 7.87 (m, 1H), 8.03 (dd, J = 8.7, 1.9 Hz, 1H), 8.16 (d, J = 8.7 Hz, 1H), 8.52 (d, J = 1.9 Hz, 1H), 9.00 (dd, J = 2.4, 1.4 Hz, 1H), 9.06 (d, J = 2.4 Hz, 1H), 9.71 (d, J = 1.4 Hz, 1H). | DMSO | >98 | R4 Temperature at 80° C. | | | |
| 890 | 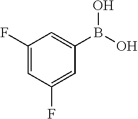 [SMILES: OB(O)c1cc(F)cc(F)c1] | 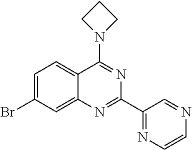 [SMILES: Brc1ccc2c(N3CCC3)nc(-c3cnccn3)nc2c1] | 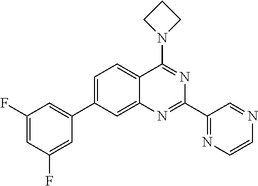 [SMILES: Fc1cc(F)cc(-c2ccc3c(N4CCC4)nc(-c4cnccn4)nc3c2)c1] | HCl | 1H NMR (400 MHz, DMSO) δ 2.66- 2.54 (m, 2H), 4.70 (brs, 2H), 5.06 (brs, 2H), 7.45 (tt, J = 9.3, 2.3 Hz, 1H), 7.60-7.51 (m, 2H), 8.05 (dd, J = 8.7,1.9 Hz, 1H), 8.15 (d, J = 8.8 Hz, 1H), 8.55 (d, J = 1.8 Hz, 1H), 8.99 (dd, J = 2.4, 1.4 Hz, 1H), 9.05 (d, J = 2.5 Hz, 1H), 9.69 (d, J = 1.4 Hz, 1H). | DMSO | >98 | R4 Temperature at 80° C. | | | |
| 891 | 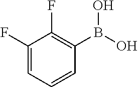 [SMILES: OB(O)c1cccc(F)c1F] | 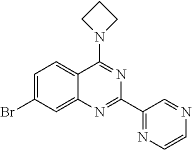 [SMILES: Brc1ccc2c(N3CCC3)nc(-c3cnccn3)nc2c1] | 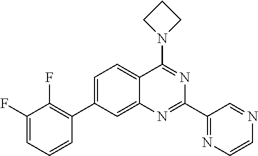 [SMILES: Fc1cccc(-c2ccc3c(N4CCC4)nc(-c4cnccn4)nc3c2)c1F] | 2HCl | 1H NMR (400 MHz, DMSO) δ 2.64- 2.54 (m, 2H), 4.68 (brs, 2H), 5.05 (brs, 2H), 7.47-7.39 (m, 1H), 7.55- 7.47 (m, 1H), 7.68-7.57 (m, 1H), 7.93-7.85 (m, 2H), 8.22 (d, J = 8.6 Hz, 1H), 8.51 (s, 1H), 8.97 (d, J = 4.9 Hz, 1H), 9.09 (d, J = 7.8 Hz, 1H), 9.66 (s, 1H). | DMSO | >98 | R4 Temperature at 80° C. | | | |
| 892 | 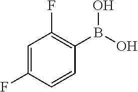 [SMILES: OB(O)c1ccc(F)cc1F] | 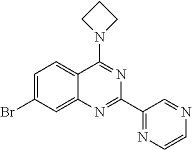 [SMILES: Brc1ccc2c(N3CCC3)nc(-c3cnccn3)nc2c1] | 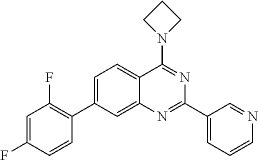 [SMILES: Fc1ccc(-c2ccc3c(N4CCC4)nc(-c4cccnc4)nc3c2)c(F)c1] | 2HCl | 1H NMR (400 MHz, DMSO) δ 2.64- 2.54 (m, 2H), 4.67 (brs, 2H), 5.05 (brs, 2H), 7.38-7.30 (m, 1H), 7.53 (ddd, J = 11.6, 9.3, 2.6 Hz, 1H), 7.76 (td, J = 8.9, 6.5 Hz, 1H), 7.88- 7.81 (m, 1H), 7.92 (dd, J = 8.0, 5.1 Hz, 1H), 8.20 (d, J = 8.7 Hz, 1H), 8.53 (s, 1H), 8.98 (dd, J = 5.1, 1.6 Hz, 1H), 9.18-9.09 (m, 1H), 9.68 (d, J = 2.2 Hz, 1H). | DMSO | >98 | R4 Temperature at 80° C. | | | |
| 893 | 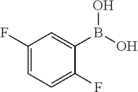 [SMILES: OB(O)c1cc(F)ccc1F] | 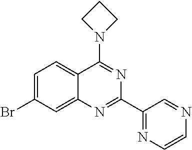 [SMILES: Brc1ccc2c(N3CCC3)nc(-c3cnccn3)nc2c1] | 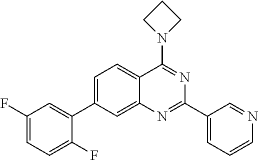 [SMILES: Fc1ccc(F)c(-c2ccc3c(N4CCC4)nc(-c4cccnc4)nc3c2)c1] | 2HCl | 1H NMR (400 MHz, DMSO) δ 2.69- 2.54 (m, 2H), 4.68 (brs, 2H), 5.05 (brs, 2H), 7.69-7.37 (m, 3H), 8.02- 7.85 (m, 2H), 8.21 (d, J = 8.3 Hz, 1H), 8.63-8.52 (m, 1H), 9.04- 8.95 (m, 1H), 9.22-9.10 (m, 1H), 9.72-9.66 (m, 1H). | DMSO | >98 | R4 Temperature at 80° C. | | | |
| 894 | 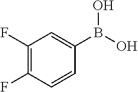 [SMILES: OB(O)c1ccc(F)c(F)c1] | 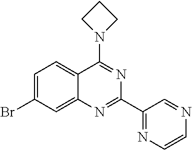 [SMILES: Brc1ccc2c(N3CCC3)nc(-c3cnccn3)nc2c1] | 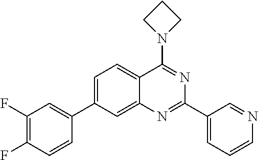 [SMILES: Fc1ccc(-c2ccc3c(N4CCC4)nc(-c4cccnc4)nc3c2)cc1F] | 2HCl | 1H NMR (400 MHz, DMSO) δ 2.66- 2.52 (m, 2H), 4.83 (brs, 4H), 7.75- 7.58 (m, 2H), 7.83 (dd, J = 7.9, 4.9 Hz, 1H), 7.93-7.86 (m, 1H), 7.97 (dd, J = 8.6, 1.9 Hz, 1H), 8.14 (d, J = 8.5 Hz, 1H), 8.58 (s, 1H), 8.92 (dd, J = 4.9, 1.6 Hz, 1H), 9.09- 9.01 (m, 1H), 9.63 (d, J = 2.3 Hz, 1H). | DMSO | >98 | R4 Temperature at 80° C. | | | |
| 895 | 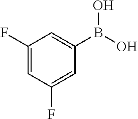 [SMILES: OB(O)c1cc(F)cc(F)c1] | 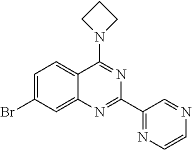 [SMILES: Brc1ccc2c(N3CCC3)nc(-c3cnccn3)nc2c1] | 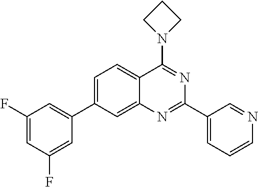 [SMILES: Fc1cc(F)cc(-c2ccc3c(N4CCC4)nc(-c4cccnc4)nc3c2)c1] | 2HCl | 1H NMR (400 MHz, DMSO) δ 2.63- 2.54 (m, 2H), 4.78 (brs, 4H), 7.39- 7.31 (m, 1H), 7.59-7.51 (m, 2H), 7.84 (dd, J = 7.9, 5.0 Hz, 1H), 8.03- 7.96 (m, 1H), 8.15 (d, J = 8.6 Hz, 1H), 8.56 (s, 1H), 8.91 (dd, J = 4.9, 1.6 Hz, 1H), 9.04 (d, J = 8.2 Hz, 1H), 9.62 (d, J = 2.1 Hz, 1H). | DMSO | >98 | R4 Temperature at 80° C. | | | |
| 896 | 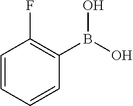 | 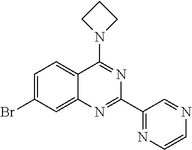 [SMILES: Brc1ccc2c(N3CCC3)nc(-c3cnccn3)nc2c1] | 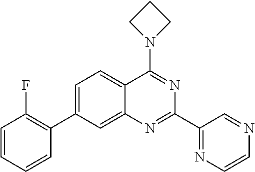 [SMILES: Fc1ccccc1-c1ccc2c(N3CCC3)nc(-c3cnccn3)nc2c1] | HCl | 1H NMR (400 MHz, DMSO) δ 2.68- 2.55 (m, 2H), 4.72 (brs, 2H), 5.08 (brs, 2H), 7.50-7.40 (m, 2H), 7.64- 7.55 (m, 1H), 7.74-7.66 (m, 1H), 7.92 (dt, J = 8.6, 1.7 Hz, 1H), 8.23 (d, J = 8.7 Hz, 1H), 8.49-8.44 (m, 1H), 9.00 (dd, J = 2.4, 1.5 Hz, 1H), 9.05 (d, J = 2.5 Hz, 1H), 9.73 (d, J = 1.4 Hz, 1H). | DMSO | >98 | R4 Temperature at 80° C. | | | |
| 897 | 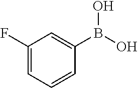 | 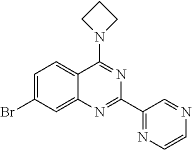 [SMILES: Brc1ccc2c(N3CCC3)nc(-c3cnccn3)nc2c1] | 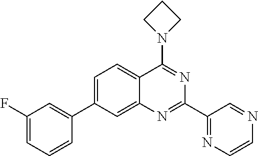 [SMILES: Fc1cccc(-c2ccc3c(N4CCC4)nc(-c4cnccn4)nc3c2)c1] | HCl | 1H NMR (400 MHz, DMSO) δ 2.69- 2.54 (m, 2H), 4.71 (brs, 2H), 5.08 (brs, 2H), 7.44-7.33 (m, 1H), 7.71- 7.62 (m, 3H), 8.06 (dd, J = 8.7, 1.9 Hz, 1H), 8.17 (d, J = 8.7 Hz, 1H), 8.56 (d, J = 1.8 Hz, 1H), 9.00 (dd, J = 2.4, 1.5 Hz, 1H), 9.06 (d, J = 2.5 Hz, 1H), 9.71 (d, J = 1.4 Hz, 1H). | DMSO | >98 | R4 Temperature at 80° C. | | | |
| 898 | 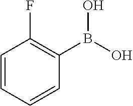 | 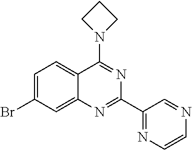 [SMILES: Brc1ccc2c(N3CCC3)nc(-c3cnccn3)nc2c1] | 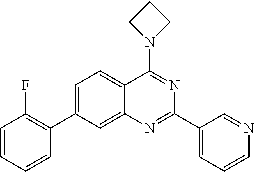 [SMILES: Fc1ccccc1-c1ccc2c(N3CCC3)nc(-c3cccnc3)nc2c1] | 2HCl | 1H NMR (400 MHz, DMSO) δ 2.64- 2.54 (m, 2H), 5.25-4.47 (m, 4H), 7.49-7.38 (m, 2H), 7.64-7.53 (m, 1H), 7.73-7.66 (m, 1H), 7.92- 7.81 (m, 2H), 8.20 (d, J = 8.7 Hz, 1H), 8.48 (s, 1H), 8.96 (dd, J = 5.0, 1.6 Hz, 1H), 9.08-9.01 (m, 1H), 9.67-9.59 (m, 1H). | DMSO | >98 | R4 Temperature at 80° C. | | | |
| 899 | 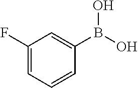 | 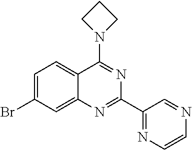 [SMILES: Brc1ccc2c(N3CCC3)nc(-c3cnccn3)nc2c1] | 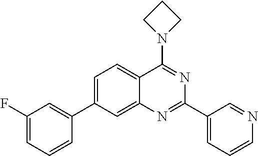 [SMILES: Fc1cccc(-c2ccc3c(N4CCC4)nc(-c4cccnc4)nc3c2)c1] | 2HCl | 1H NMR (400 MHz, DMSO) δ 2.65- 2.50 (m, 2H), 4.93 (brs, 4H), 7.47- 7.34 (m, 1H), 7.77-7.61 (m, 3H), 8.00-7.87 (m, 1H), 8.11-7.99 (m, 1H), 8.25-8.13 (m, 1H), 8.73 (s, 1H), 9.08-8.93 (m, 1H), 9.26- 9.09 (m, 1H), 9.71 (s, 1H). | DMSO | >98 | R4 Temperature at 80° C. | | | |
| 900 | 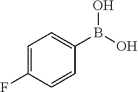 | 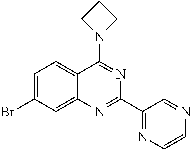 [SMILES: Brc1ccc2c(N3CCC3)nc(-c3cnccn3)nc2c1] | 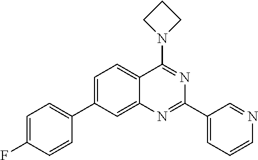 [SMILES: Fc1ccc(-c2ccc3c(N4CCC4)nc(-c4cccnc4)nc3c2)cc1] | 2HCl | 1H NMR (400 MHz, DMSO) δ 2.66- 2.54 (m, 2H), 4.57 (brs, 2H), 5.04 (brs, 2H), 7.53-7.38 (m, 2H), 7.95- 7.81 (m, 3H), 8.05-7.98 (m, 1H), 8.17 (d, J = 8.6 Hz, 1H), 8.65 (s, 1H), 8.97 (d, J = 5.1 Hz, 1H), 9.10 (d, J = 7.8 Hz, 1H), 9.68 (s, 1H). | DMSO | >98 | R4 Temperature at 80° C. | | | |
| 901 | 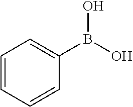 | 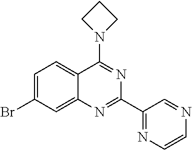 [SMILES: Brc1ccc2c(N3CCC3)nc(-c3cnccn3)nc2c1] | 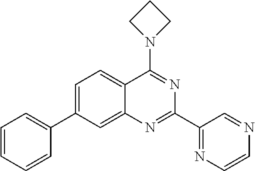 [SMILES: c1ccc(-c2ccc3c(N4CCC4)nc(-c4cnccn4)nc3c2)cc1] | HCl | 1H NMR (400 MHz, DMSO) δ 2.66- 2.53 (m, 2H), 4.68 (brs, 2H), 5.08 (brs, 2H), 7.58-7.50 (m, 1H), 7.68- 7.58 (m, 2H), 7.85-7.77 (m, 2H), 8.03 (dd, J = 8.7, 1.8 Hz, 1H), 8.16 (d, J = 8.7 Hz, 1H), 8.57 (d, J = 1.8 Hz, 1H), 8.99 (dd, J = 2.4, 1.5 Hz, 1H), 9.06 (d, J = 2.4 Hz, 1H), 9.69 (d, J = 1.4 Hz, 1H). | DMSO | >98 | R4 Temperature at 80° C. | | | |
| 902 | 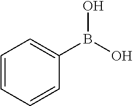 | 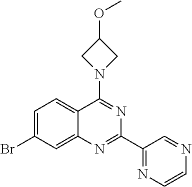 [SMILES: COC1CN(c2nc(-c3cnccn3)nc3cc(Br)ccc23)C1] | 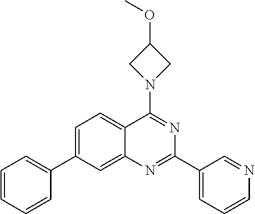 [SMILES: COC1CN(c2nc(-c3cccnc3)nc3cc(-c4ccccc4)ccc23)C1] | 2HCl | 1H NMR (400 MHz, DMSO) δ 3.36 (s, 3H), 4.51 (dt, J = 6.5, 3.0 Hz, 1H), 6.32-4.64 (m, 4H), 7.56- 7.51 (m, 1H), 7.64-7.58 (m, 2H), 7.85-7.80 (m, 2H), 7.92-7.85 (m, 1H), 7.99 (dd, J = 8.7, 1.9 Hz, 1H), 8.18 (d, J = 8.7 Hz, 1H), 8.64 (s, 1H), 8.96 (dd, J = 5.1, 1.6 Hz, 1H), 9.17-9.06 (m, 1H), 9.67 (dd, J = 2.2, 0.8 Hz, 1H). | DMSO | >98 | R4 Temperature at 80° C. | | | |
| 903 | 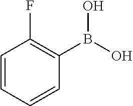 | 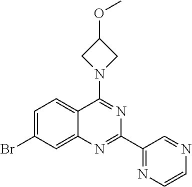 [SMILES: COC1CN(c2nc(-c3cnccn3)nc3cc(Br)ccc23)C1] | 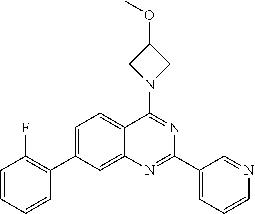 [SMILES: COC1CN(c2nc(-c3cccnc3)nc3cc(-c4ccccc4F)ccc23)C1] | 2HCl | 1H NMR (400 MHz, DMSO) δ 3.36 (s, 3H), 4.56-4.46 (m, 1H), 6.07- 4.59 (m, 4H), 7.50-7.37 (m, 2H), 7.63-7.53 (m, 1H), 7.69 (td, J = 7.8, 1.7 Hz, 1H), 7.86 (dt, J = 8.6, 1.7 Hz, 1H), 7.90 (dd, J = 8.1, 5.2 Hz, 1H), 8.22 (d, J = 8.7 Hz, 1H), 8.48 (s, 1H), 8.96 (dd, J = 5.1, 1.5 Hz, 1H), 9.11 (d, J = 8.0 Hz, 1H), 9.66 (d, J = 2.1 Hz, 1H). | DMSO | >98 | R4 Temperature at 80° C. | | | |
| 904 | 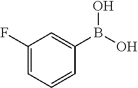 | 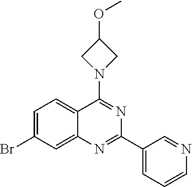 [SMILES: COC1CN(c2nc(-c3cccnc3)nc3cc(Br)ccc23)C1] | 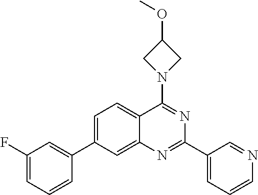 [SMILES: COC1CN(c2nc(-c3cccnc3)nc3cc(-c4cccc(F)c4)ccc23)C1] | 2HCl | 1H NMR (400 MHz, DMSO) δ 3.36 (s, 3H), 4.59-4.44 (m, 1H), 5.36- 4.62 (m, 4H), 7.45-7.29 (m, 1H), 7.72-7.60 (m, 3H), 7.97-7.89 (m, 1H), 8.02 (d, J = 8.7 Hz, 1H), 8.18 (d, J = 8.6 Hz, 1H), 8.67 (s, 1H), 8.98 (d, J = 5.0 Hz, 1H), 9.17 (d, J = 8.0 Hz, 1H), 9.69 (s, 1H). | DMSO | >98 | R4 Temperature at 80° C. | | | |
| 905 | 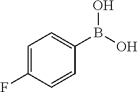 | 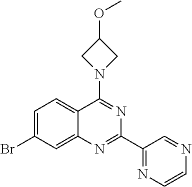 [SMILES: COC1CN(c2nc(-c3cnccn3)nc3cc(Br)ccc23)C1] | 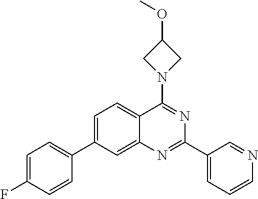 [SMILES: COC1CN(c2nc(-c3cccnc3)nc3cc(-c4ccc(F)cc4)ccc23)C1] | 2HCl | 1H NMR (400 MHz, DMSO) δ 3.36 (s, 3H), 4.56-4.43 (m, 1H), 5.34- 4.67 (m, 4H), 7.50-7.34 (m, 2H), 7.92-7.82 (m, 3H), 7.96 (dd, J = 8.7, 1.9 Hz, 1H), 8.15 (d, J = 8.7 Hz, 1H), 8.63 (s, 1H), 8.96 (dd, J = 5.1, 1.5 Hz, 1H), 9.19-9.10 (m, 1H), 9.67 (dd, J = 2.1, 0.8 Hz, 1H). | DMSO | >98 | R4 Temperature at 80° C. | | | |
| 906 | 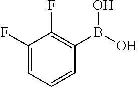 [SMILES: OB(O)c1cccc(F)c1F] | 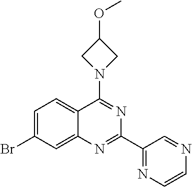 [SMILES: COC1CN(c2nc(-c3cnccn3)nc3cc(Br)ccc23)C1] | 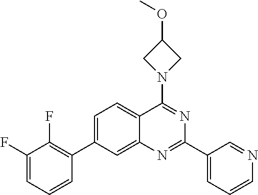 [SMILES: COC1CN(c2nc(-c3cccnc3)nc3cc(-c4cccc(F)c4F)ccc23)C1] | 2HCl | 1H NMR (400 MHz, DMSO) δ 4.55- 4.45 (m, 1H), 5.43-4.58 (m, 4H), 7.46-7.37 (m, 1H), 7.55-7.48 (m, 1H), 7.69-7.56 (m, 1H), 7.86 (dt, J = 8.6,1.7 Hz, 1H), 7.93 (dd, J = 8.1, 5.1 Hz, 1H), 8.22 (d, J = 8.7 Hz, 1H), 8.51 (s, 1H), 8.98 (dd, J = 5.2, 1.5 Hz, 1H), 9.23-9.12 (m, 1H), 9.71-9.63 (m, 1H). | DMSO | >98 | R4 Temperature at 80° C. | | | |
| 907 | 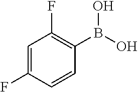 [SMILES: OB(O)c1ccc(F)cc1F] | 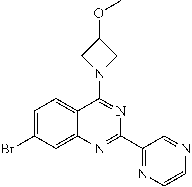 [SMILES: COC1CN(c2nc(-c3cnccn3)nc3cc(Br)ccc23)C1] | 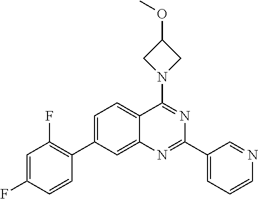 [SMILES: COC1CN(c2nc(-c3cccnc3)nc3cc(-c4ccc(F)cc4F)ccc23)C1] | 2HCl | 1H NMR (400 MHz, DMSO) δ 3.36 (s, 3H), 4.55-4.47 (m, 1H), 5.28- 4.62 (m, 4H), 7.51-7.37 (m, 1H), 7.92-7.82 (m, 3H), 7.96 (dd, J = 8.7, 1.9 Hz, 1H), 8.15 (d, J = 8.7 Hz, 1H), 8.63 (s, 1H), 8.96 (dd, J = 5.1, 1.5 Hz, 1H), 9.13 (dt, J = 8.1, 1.9 Hz, 1H), 9.67 (dd, J = 2.1, 0.8 Hz, 1H). | DMSO | >98 | R4 Temperature at 80° C. | | | |
| 908 | 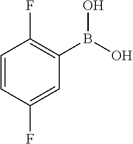 [SMILES: OB(O)c1cc(F)ccc1F] | 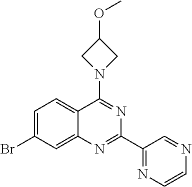 [SMILES: COC1CN(c2nc(-c3cnccn3)nc3cc(Br)ccc23)C1] | 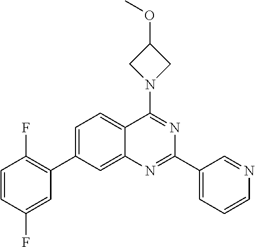 [SMILES: COC1CN(c2nc(-c3cccnc3)nc3cc(-c4cc(F)ccc4F)ccc23)C1] | 2HCl | 1H NMR (400 MHz, DMSO) δ 3.36 (s, 3H), 4.58-4.45 (m, 1H), 5.34- 4.68 (m, 4H), 7.48-7.37 (m, 1H), 7.56-7.48 (m, 1H), 7.67-7.56 (m, 1H), 7.87 (d, J = 8.5 Hz, 1H), 8.00-7.91 (m, 1H), 8.21 (d, J = 8.6 Hz, 1H), 8.51 (s, 1H), 8.99 (d, J = 5.0 Hz, 1H), 9.19 (d, J = 7.9 Hz, 1H), 9.69 (s, 1H). | DMSO | >98 | R4 Temperature at 80° C. | | | |
| 909 | 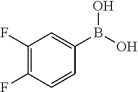 [SMILES: OB(O)c1ccc(F)c(F)c1] | 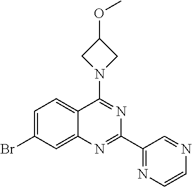 [SMILES: COC1CN(c2nc(-c3cnccn3)nc3cc(Br)ccc23)C1] | 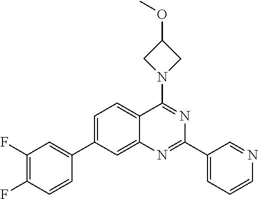 [SMILES: COC1CN(c2nc(-c3cccnc3)nc3cc(-c4ccc(F)c(F)c4)ccc23)C1] | 2HCl | 1H NMR (400 MHz, DMSO) δ 3.36 (s, 3H), 4.61-4.39 (m, 1H), 7.79- 7.62 (m, 2H), 8.00 (d, J = 19.8 Hz, 3H), 8.19 (s, 1H), 8.61 (s, 1H), 9.03- 8.97 (m, 1H), 9.22-9.16 (m, 1H), 9.70 (s, 1H). | DMSO | >98 | R4 Temperature at 80° C. | | | |
| 910 | 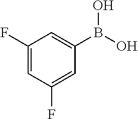 [SMILES: OB(O)c1cc(F)cc(F)c1] | 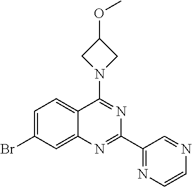 [SMILES: COC1CN(c2nc(-c3cnccn3)nc3cc(Br)ccc23)C1] | 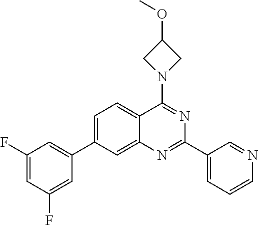 [SMILES: COC1CN(c2nc(-c3cccnc3)nc3cc(-c4cc(F)cc(F)c4)ccc23)C1] | 2HCl | 1H NMR (400 MHz, DMSO) δ 3.36 (s, 3H), 4.55-4.46 (m, 1H), 5.31- 4.64 (m, 4H), 7.41 (tt, J = 9.1, 2.2 Hz, 1H), 7.64-7.51 (m, 2H), 7.96- 7.88 (m, 1H), 7.98 (dd, J = 8.7, 1.9 Hz, 1H), 8.13 (d, J = 8.7 Hz, 1H), 8.60 (s, 1H), 8.97 (dd, J = 5.2, 1.5 Hz, 1H), 9.16 (d, J = 8.0 Hz, 1H), 9.68-9.62 (m, 1H). | DMSO | >98 | R4 Temperature at 80° C. | | | |
| 911 | 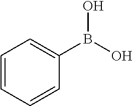 | 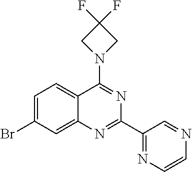 [SMILES: FC1(F)CN(c2nc(-c3cnccn3)nc3cc(Br)ccc23)C1] | 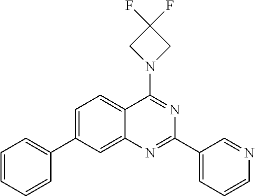 [SMILES: FC1(F)CN(c2nc(-c3cccnc3)nc3cc(-c4ccccc4)ccc23)C1] | 2HCl | 1H NMR (400 MHz, DMSO) δ 5.37- 5.12 (m, 4H), 7.55-7.47 (m, 1H), 7.65-7.56 (m, 2H), 7.92-7.84 (m, 2H), 8.07-7.95 (m, 2H), 8.14 (d, J = 8.7 Hz, 1H), 8.40 (s, 1H), 8.99 (dd, J = 5.3, 1.6 Hz, 1H), 9.30- 9.21 (m, 1H), 9.74-9.66 (m, 1H). | DMSO | >98 | R4 Temperature at 80° C. | | | |
| 912 | 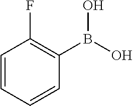 | 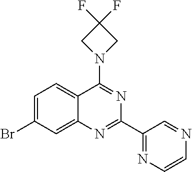 [SMILES: FC1(F)CN(c2nc(-c3cnccn3)nc3cc(Br)ccc23)C1] | 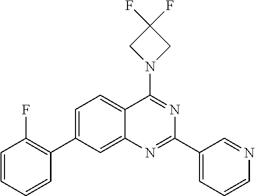 [SMILES: Fc1ccccc1-c1ccc2c(N3CC(F)(F)C3)nc(-c3cccnc3)nc2c1] | 2HCl | 1H NMR (400 MHz, DMSO) δ 5.24 (t, J = 12.1 Hz, 4H), 7.48-7.35 (m, 2H), 7.63-7.52 (m, 1H), 7.73 (td, J = 7.8,1.7 Hz, 1H), 7.85 (dt, J = 8.6, 1.8 Hz, 1H), 8.07 (dd, J = 8.2, 5.4 Hz, 1H), 8.16 (d, J = 8.6 Hz, 1H), 8.30 (s, 1H), 9.01 (dd, J = 5.4, 1.5 Hz, 1H), 9.31 (dt, J = 8.2, 1.7 Hz, 1H), 9.71 (d, J = 2.1 Hz, 1H). | DMSO | >98 | R4 Temperature at 80° C. | | | |
| 913 | 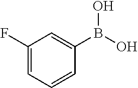 | 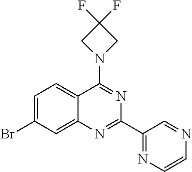 [SMILES: FC1(F)CN(c2nc(-c3cnccn3)nc3cc(Br)ccc23)C1] | 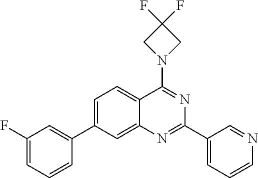 [SMILES: Fc1cccc(-c2ccc3c(N4CC(F)(F)C4)nc(-c4cccnc4)nc3c2)c1] | 2HCl | 1H NMR (400 MHz, DMSO) δ 5.23 (t, J = 11.8 Hz, 4H), 7.41-7.27 (m, 1H), 7.68-7.53 (m, 1H), 7.81- 7.68 (m, 2H), 8.00 (dd, J = 8.7, 1.9 Hz, 1H), 8.19-8.03 (m, 2H), 8.47 (s, 1H), 9.03 (dd, J = 5.4, 1.5 Hz, 1H), 9.33 (dt, J = 8.1, 1.8 Hz, 1H), 9.70 (d, J = 1.9 Hz, 1H). | DMSO | >98 | R4 Temperature at 80° C. | | | |
| 914 | 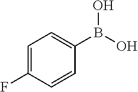 | 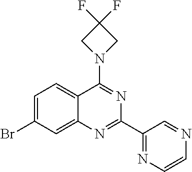 [SMILES: FC1(F)CN(c2nc(-c3cnccn3)nc3cc(Br)ccc23)C1] | 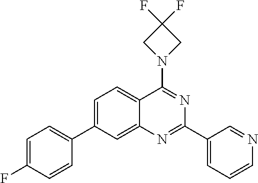 [SMILES: Fc1ccc(-c2ccc3c(N4CC(F)(F)C4)nc(-c4cccnc4)nc3c2)cc1] | 2HCl | 1H NMR (400 MHz, DMSO) δ 5.25 (t, J = 11.8 Hz, 4H), 7.51-7.36 (m, 2H), 8.01-7.90 (m, 3H), 8.08 (dd, J = 8.1, 5.3 Hz, 1H), 8.13 (d, J = 8.7 Hz, 1H), 8.46 (s, 1H), 9.03 (dd, J = 5.4, 1.5 Hz, 1H), 9.33 (dt, J = 8.1, 1.7 Hz, 1H), 9.72 (d, J = 2.0 Hz, 1H). | DMSO | >98 | R4 Temperature at 80° C. | | | |
| 915 | 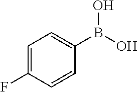 | 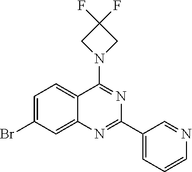 [SMILES: FC1(F)CN(c2nc(-c3cccnc3)nc3cc(Br)ccc23)C1] | 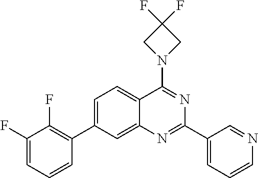 [SMILES: Fc1cccc(-c2ccc3c(N4CC(F)(F)C4)nc(-c4cccnc4)nc3c2)c1F] | 2HCl | 1H NMR (400 MHz, DMSO) δ 5.23 (t, J = 12.2 Hz, 4H), 7.48-7.37 (m, 1H), 7.67-7.51 (m, 2H), 7.85 (dt, J = 8.6, 1.8 Hz, 1H), 8.10 (dd, J = 8.1, 5.4 Hz, 1H), 8.17 (d, J = 8.7 Hz, 1H), 8.30 (s, 1H), 9.03 (dd, J = 5.5, 1.5 Hz, 1H), 9.34 (dt, J = 8.3, 1.8 Hz, 1H), 9.71 (d, 3 = 1.9 Hz, 1H). | DMSO | >98 | R4 Temperature at 80° C. | | | |
| 916 | 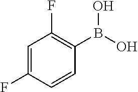 [SMILES: OB(O)c1ccc(F)cc1F] | 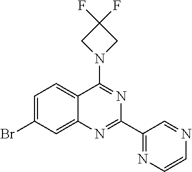 [SMILES: FC1(F)CN(c2nc(-c3cnccn3)nc3cc(Br)ccc23)C1] | 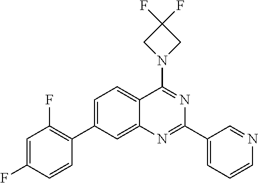 [SMILES: Fc1ccc(-c2ccc3c(N4CC(F)(F)C4)nc(-c4cccnc4)nc3c2)c(F)c1] | 2HCl | 1H NMR (400 MHz, DMSO) δ 5.23 (t, J = 11.9 Hz, 4H), 7.37-7.25 (m, 1H), 7.50 (ddd, J = 11.6, 9.3, 2.6 Hz, 1H), 7.85-7.76 (m, 2H), 8.09 (dd, J = 8.1, 5.4 Hz, 1H), 8.14 (d, J = 8.7 Hz, 1H), 8.28 (s, 1H), 9.02 (dd, J = 5.5, 1.5 Hz, 1H), 9.33 (dt, J = 8.3, 1.6 Hz, 1H), 9.70 (d, J = 1.9 Hz, 1H). | DMSO | >98 | R4 Temperature at 80° C. | | | |
| 917 | 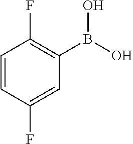 [SMILES: OB(O)c1cc(F)ccc1F] | 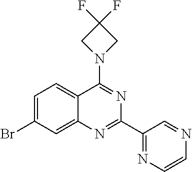 [SMILES: FC1(F)CN(c2nc(-c3cnccn3)nc3cc(Br)ccc23)C1] | 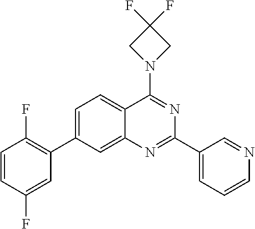 [SMILES: Fc1ccc(F)c(-c2ccc3c(N4CC(F)(F)C4)nc(-c4cccnc4)nc3c2)c1] | 2HCl | 1H NMR (400 MHz, DMSO) δ 5.30- 5.12 (m, 4H), 7.45-7.36 (m, 1H), 7.57-7.45 (m, 1H), 7.64 (ddd, J = 9.1, 6.1, 3.1 Hz, 1H), 7.85 (dt, J = 8.6, 1.8 Hz, 1H), 8.10 (dd, J = 8.1, 5.4 Hz, 1H), 8.15 (d, J = 8.7 Hz, 1H), 8.29 (s, 1H), 9.02 (dd, J = 5.5, 1.5 Hz, 1H), 9.33 (dt, J = 8.2, 1.8 Hz, 1H), 9.70 (d, J = 1.9 Hz, 1H). | DMSO | >98 | R4 Temperature at 80° C. | | | |
| 918 | 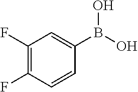 [SMILES: OB(O)c1ccc(F)c(F)c1] | 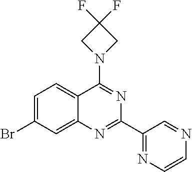 [SMILES: FC1(F)CN(c2nc(-c3cnccn3)nc3cc(Br)ccc23)C1] | 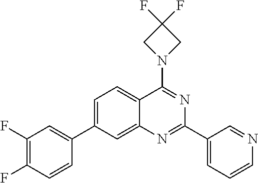 [SMILES: Fc1ccc(-c2ccc3c(N4CC(F)(F)C4)nc(-c4cccnc4)nc3c2)cc1F] | 2HCl | 1H NMR (400 MHz, DMSO) δ 5.22 (t, J = 11.8 Hz, 4H), 7.64 (dt, J = 10.6, 8.5 Hz, 1H), 7.82-7.74 (m, 1H), 8.16-7.95 (m, 4H), 8.40 (s, 1H), 9.02 (dd, J = 5.4, 1.6 Hz, 1H), 9.36-9.26 (m, 1H), 9.69 (d, J = 2.0 Hz, 1H). | DMSO | >98 | R4 Temperature at 80° C. | | | |
| 919 | 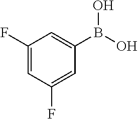 [SMILES: OB(O)c1cc(F)cc(F)c1] | 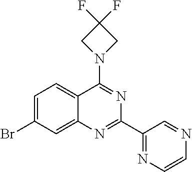 [SMILES: FC1(F)CN(c2nc(-c3cnccn3)nc3cc(Br)ccc23)C1] | 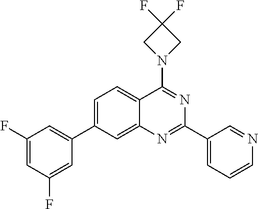 [SMILES: Fc1cc(F)cc(-c2ccc3c(N4CC(F)(F)C4)nc(-c4cccnc4)nc3c2)c1] | 2HCl | 1H NMR (400 MHz, DMSO) δ 5.20 (t, J = 12.0 Hz, 4H), 7.39 (tt, J = 9.1, 2.2 Hz, 1H), 7.77-7.62 (m, 2H), 8.00 (dd, J = 8.8, 2.0 Hz, 1H), 8.19-8.05 (m, 2H), 8.42 (s, 1H), 9.07-8.98 (m, 1H), 9.39-9.23 (m, 1H), 9.68 (d, J = 1.9 Hz, 1H). | DMSO | >98 | R4 Temperature at 80° C. | | | |

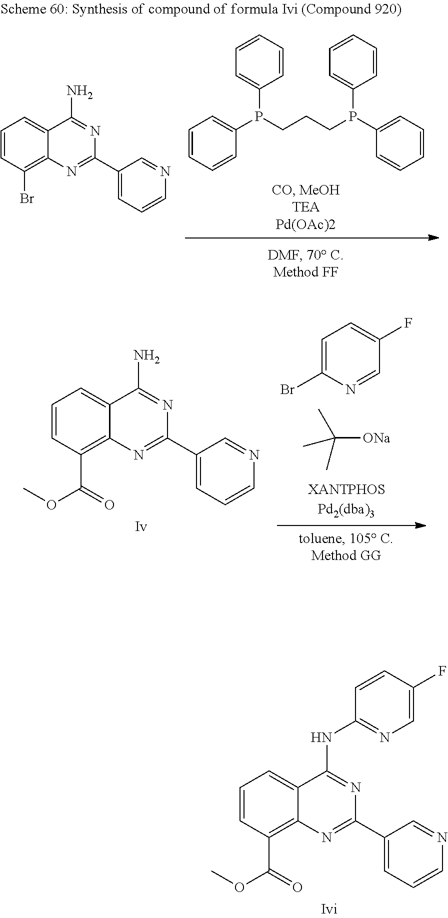

### Method FF: Methyl 4-amino-2-(pyridin-3-yl)quinazoline-8-carboxylate (lv)

To a solution of 8-bromo-2-(pyridin-3-yl)quinazolin-4-amine (2.17 g, 7.21 mmol) in THF (20 ml) were added Methanol (10 ml), TEA (10 ml), 1,3-Bis(diphenylphosphino)propane (0.446 g, 1.081 mmol), Pd(OAc)₂ (0.162 g, 0.721 mmol) and stirred at 70° C. under carbon monoxide atmosphere for 7 h. To the reaction was added water, filtered, extracted with ethyl acetate and washed with water. The obtained ethyl acetate phase was charged directly onto the column chromatography (NH-silica gel, ethyl acetate), purified and concentrated in vacuo to give methyl 4-amino-2-(pyridin-3-yl)quinazoline-8-carboxylate (1.27 g, 62%) as a pale orange solid. ¹H NMR (400 MHz, DMSO) δ 3.96 (s, 3H), 7.55 (dd, J=8.2, 7.3 Hz, 2H), 8.00 (dd, J=7.2, 1.3 Hz, 1H), 8.12 (brs, 2H), 8.42 (dd, J=8.3, 1.4 Hz, 1H), 8.72-8.62 (m, 2H), 9.55 (dd, J=2.0, 0.9 Hz, 1H).

### Method GG: Methyl 4-(5-fluoropyridin-2-ylamino)-2-(pyridin-3-yl)quinazoline-8-carboxylate (lvi)

A flask was charged with methyl 4-amino-2-(pyridin-3-yl)quinazoline-8-carboxylate (400 mg, 1.427 mmol), 2-Bromo-5-fluoropyridine (301 mg, 1.713 mmol), XANTPHOS (165 mg, 0.285 mmol), Sodium-t-butoxide (0.186 ml, 2.141 mmol) and Pd2(dba)₃ (131 mg, 0.143 mmol). The mixture was suspended in toluene (15 ml) and the reaction was heated at 105° C. for 8 h. The volatiles were evaporated in vacuo, dissolved in ethyl acetate and charged directly onto the column chromatography (NH-silica gel, ethyl acetate) for purification. The fraction was concentrated and the residue was washed with a small amount of ethyl acetate, filtered and dried to give Methyl 4-(5-fluoropyridin-2-ylamino)-2-(pyridin-3-yl)quinazoline-8-carboxylate (177 mg, 33%) as a pale yellow powder. ¹H NMR (400 MHz, CDCl₃) δ 4.10 (s, 3H), 7.46 (ddd, J=7.9, 4.8, 0.9 Hz, 1H), 7.68-7.59 (m, 2H), 8.12 (dd, J=8.5, 1.3 Hz, 1H), 8.16 (dd, J=7.3, 1.3 Hz, 1H), 8.25 (d, J=2.9 Hz, 1H), 8.36 (s, 1H), 8.74 (dd, J=4.9, 1.7 Hz, 1H), 8.81-8.79 (m, 1H), 8.85-8.81 (m, 1H), 9.74 (dd, J=2.2, 0.9 Hz, 1H).

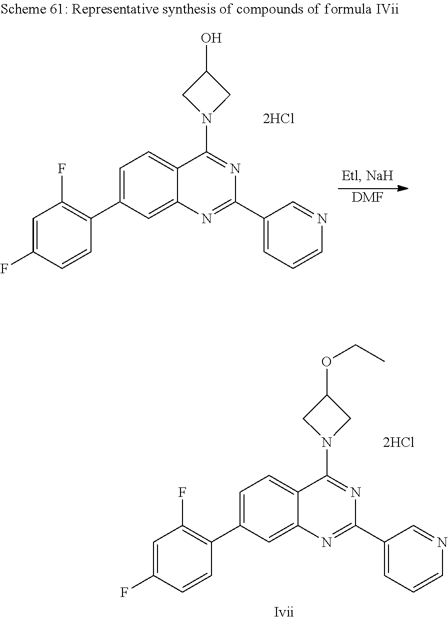

### Method GG: 7-(2,4-Difluorophenyl)-4-(3-ethoxyazetidin-1-yl)-2-(pyridin-3-yl)quinazoline, 2HCl of formula lvii (Compound 921)

To a solution of 1-(7-(2,4-difluorophenyl)-2-(pyridin-3-yl)quinazolin-4-yl)azetidin-3-ol, 2HCl (300 mg, 0.648 mmol) in DMF (10 ml) were added NaH (113 mg, 2.59 mmol) and Ethyliodide (0.067 ml, 0.842 mmol) and stirred at room temperature for 3 h. Water was added, extracted with ethyl acetate, washed with water, dried over MgSO4, filtered and concentrated in vacuo. To the residue was added 6N HCl (1 ml) and volatiles were evaporated. The residue was dissolved in i-PrOH (1 ml) and the generated powder was obtained by filtration and dried at 60° C. The desired product, 7-(2,4-Difluorophenyl)-4-(3-ethoxyazetidin-1-yl)-2-(pyridin-3-yl)quinazoline, was obtained (112 mg, 35%) as a pale yellow powder. ¹H NMR (400 MHz, DMSO) δ 1.21 (t, J=7.0 Hz, 3H), 3.57 (q, J=7.0 Hz, 2H), 5.42-4.22 (m, 5H), 7.33 (td, J=8.7, 3.0 Hz, 1H), 7.52 (ddd, J=11.6, 9.3, 2.6 Hz, 1H), 7.76 (td, J=8.9, 6.6 Hz, 1H), 7.86-7.80 (m, 1H), 7.91 (dd, J=8.2, 5.1 Hz, 1H), 8.20 (d, J=8.7 Hz, 1H), 8.45 (s, 1H), 8.97 (dd, J=5.1, 1.5 Hz, 1H), 9.19-9.06 (m, 1H), 9.66 (dd, J=2.2, 0.8 Hz, 1H).

TABLE 33

| | | | | | | | | Method | | | |
|---|---|---|---|---|---|---|---|---|---|---|---|
| | Starting | Starting | | Salt | | ¹H NMR | Purity | of | | Retention | LCMS |
| Number | Material 1 | Material 2 | Product | type | ¹H NMR | Solvent | percent | Coupling | LCMS | Time | Method |
| 922 | 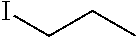 | 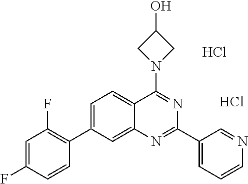 [SMILES: OC1CN(c2nc(-c3cccnc3)nc3cc(-c4ccc(F)cc4F)ccc23)C1 ; Cl ; Cl] | 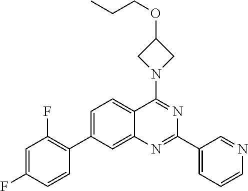 [SMILES: CCCOC1CN(c2nc(-c3cccnc3)nc3cc(-c4ccc(F)cc4F)ccc23)C1] | 2HCl | ¹H NMR (400 MHz, DMSO) δ 0.93 (t, J = 7.4 Hz, 3H), 1.70-1.53 (m, 2H), 3.47 (t, J = 6.6 Hz, 2H), 5.51- 4.02 (m, 5H), 7.33 (td, J = 8.3, 2.1 Hz, 1H), 7.52 (ddd, J= 11.6, 9.3, 2.6 Hz, 1H), 7.77 (td, J= 8.9, 6.5 Hz, 1H), 7.86-7.81 (m, 1H), 7.90 (dd, J= 8.0, 5.1 Hz, 1H), 8.21 (d, | DMSO | >98 | GG | | | |
| | | | | | J = 8.7 Hz, 1H), 8.42 | | | | | | |
| | | | | | (s, 1H), 8.96 | | | | | | |
| | | | | | (dd, J = 5.2, 1.6 | | | | | | |
| | | | | | Hz, 1H), 9.17- | | | | | | |
| | | | | | 9.06 (m, 1H), 9.65 | | | | | | |
| | | | | | (d, J = 1.7 Hz, | | | | | | |
| | | | | | 1H). | | | | | | |

TABLE 34

| | | | | | | | Puri- | Method | | Re | |
|---|---|---|---|---|---|---|---|---|---|---|---|
| | | | | | | | ty | of | | ten- | LCMS |
| Num- | Starting | Starting | | Salt | | ¹H NMR | per- | Cou- | | tion | Meth- |
| ber | Material 1 | Material 2 | Product | type | ¹H NMR | Solvent | cent | pling | LCMS | Time | od |
| 923 | 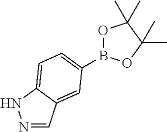 [SMILES: CC1(C)OB(c2ccc3[nH]ncc3c2)OC1(C)C] | 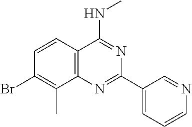 [SMILES: CNc1nc(-c2cccnc2)nc2c(C)c(Br)ccc12] | 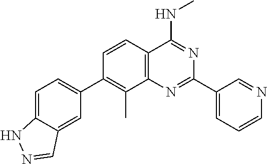 [SMILES: CNc1nc(-c2cccnc2)nc2c(C)c(-c3ccc4[nH]ncc4c3)ccc12] | 3HCl | 1H NMR (400 MHz, DMSO) δ 9.73 (d, J = 2.0 Hz, 1H), 9.44 (d, J = 8.2 Hz, 1H), 9.02 (d, J = 5.5 Hz, 1H), 8.87 (s, 1H), 8.35- 8.09 (m, 3H), 7.83 (t, J = 1.2, 1.2 Hz, 1H), 7.74- 7.60 (m, 1H), 7.54 | DMSO | >98 | Method R3 | | | |
| | | | | | (d, J = 8.5 Hz, | | | | | | |
| | | | | | 1H), 7.44 (dd, J = | | | | | | |
| | | | | | 8.6, 1.6 Hz, 1H), | | | | | | |
| | | | | | 3.22 (d, J = 3.9 | | | | | | |
| | | | | | Hz, 3H), 2.67 (s, | | | | | | |
| | | | | | 3H). 1H of | | | | | | |
| | | | | | 3HCl was not | | | | | | |
| | | | | | observed. | | | | | | |
| 924 | 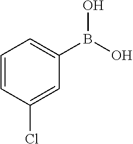 | 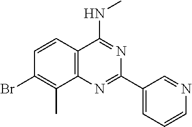 [SMILES: CNc1nc(-c2cccnc2)nc2c(C)c(Br)ccc12] | 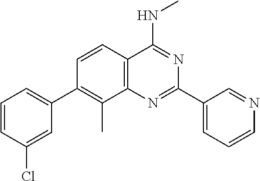 [SMILES: CNc1nc(-c2cccnc2)nc2c(C)c(-c3cccc(Cl)c3)ccc12] | 2HCl | 1H NMR (400 MHz, DMSO) δ 9.73 (d, J = 1.9 Hz, 1H), 9.30 (dt, J = 8.0, 1.8, 1.8 Hz, 1H), 8.92 (dd, J = 5.4, 1.6 Hz, 1H), 8.72 (d, J = 5.0 Hz, 1H), 8.18 (d, J = 8.5 Hz, 1H), 7.51-7.36 (m, 2H), 8.02 (dd, J = | DMSO | >98 | Method R3 Temper- ature at 80° C. | | | |
| | | | | | 8.1, 5.4 Hz, 1H), | | | | | | |
| | | | | | 7.68-7.47 (m, | | | | | | |
| | | | | | 3H), 3.20 (d, J = | | | | | | |
| | | | | | 4.3 Hz, 3H), 2.64 | | | | | | |
| | | | | | (s, 3H). □H | | | | | | |
| | | | | | of 2HCl was not | | | | | | |
| | | | | | observed. | | | | | | |
| 925 | 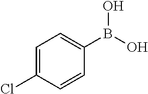 | 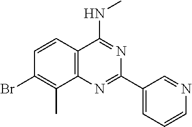 [SMILES: CNc1nc(-c2cccnc2)nc2c(C)c(Br)ccc12] | 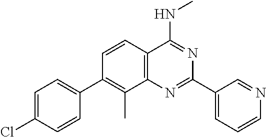 [SMILES: CNc1nc(-c2cccnc2)nc2c(C)c(-c3ccc(Cl)cc3)ccc12] | 2HCl | H NMR (400 MHz, DMSO) δ 9.73 (d, J = 1.9 Hz, 1H), 9.41 (d, J = 8.0 H7, 1H), 8.99 (dd, J = 5.6, 1.4 Hz, 1H), 8.82 (s, 1H), 8.22 (d, J = 8.6 Hz, 1H), 8.13 (dd, J = | DMSO | >98 | Method R3 Temper- ature at 80° C. | | | |
| | | | | | 8.1, 5.6 Hz, 1H), | | | | | | |
| | | | | | 7.65-7.54 (m, | | | | | | |
| | | | | | 2H), 7.54-7.38 | | | | | | |
| | | | | | (m, 3H), 3.20 (d, | | | | | | |
| | | | | | J = 4.2 Hz, 3H), | | | | | | |
| | | | | | 2.64 (s, 3H). 1H | | | | | | |
| | | | | | of 2HCl was not | | | | | | |
| | | | | | observed. | | | | | | |
| 926 | 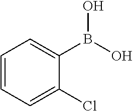 | 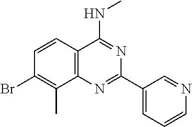 [SMILES: CNc1nc(-c2cccnc2)nc2c(C)c(Br)ccc12] | 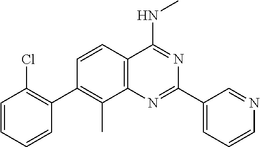 [SMILES: CNc1nc(-c2cccnc2)nc2c(C)c(-c3ccccc3Cl)ccc12] | free | 1H NMR (400 MHz, DMSO) δ 9.68 (dd, J = 2.2, 0.9 Hz, 1H), 8.82 (dt, J = 8.0, 2.0, 2.0 Hz, 1H), 8.69 (dd, J = 4.8, 1.7 Hz, 1H), 8.48 (d, J = 5.1 Hz, 1H), 8.13 (d, J = 8.5 Hz, 1H), | DMSO | >98 | Method R3 Temper- ature at 80° C. | | | |
| | | | | | 7.64 (dd, J = 5.8, | | | | | | |
| | | | | | 3.5 Hz, 1H), 7.55 | | | | | | |
| | | | | | (ddd, J = 7.9, 4.7, | | | | | | |
| | | | | | 0.9 Hz, 1H), 7.51- | | | | | | |
| | | | | | 7.44 (m, 2H), | | | | | | |
| | | | | | 7.44-7.36 (m, | | | | | | |
| | | | | | 1H), 7.28 (d, J = | | | | | | |
| | | | | | 8.4 Hz, 1H), 3.18 | | | | | | |
| | | | | | (d, J = 4.4 Hz, | | | | | | |
| | | | | | 3H), 2.44 (s, 3H). | | | | | | |
| 927 | 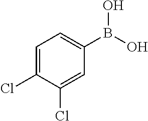 [SMILES: OB(O)c1ccc(Cl)c(Cl)c1] | 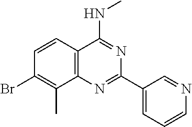 [SMILES: CNc1nc(-c2cccnc2)nc2c(C)c(Br)ccc12] | 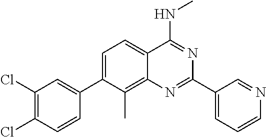 [SMILES: CNc1nc(-c2cccnc2)nc2c(C)c(-c3ccc(Cl)c(Cl)c3)ccc12] | 2HCl | 1H NMR (400 MHz, DMSO) δ 9.73 (d, J = 2.0 Hz, 1H), 9.39 (s, 1H), 8.98 (d, J = 3.3 Hz, 1H), 8.80 (s, 1H), 8.22 (d, J = 8.6 Hz, 1H), 8.11 (s, 1H), 7.88-7.72 | DMSO | >98 | Method R3 Temper- ature at 80° C. | | | |
| | | | | | (m, 2H), 7.53- | | | | | | |
| | | | | | 7.39 (m, 2H), 3.20 | | | | | | |
| | | | | | (d, J = 4.2 Hz, | | | | | | |
| | | | | | 3H), 2.64 (s, 3H). | | | | | | |
| | | | | | 1H of 2HCl was | | | | | | |
| | | | | | not observed. | | | | | | |
| 928 | 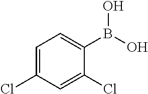 [SMILES: OB(O)c1ccc(Cl)cc1Cl] | 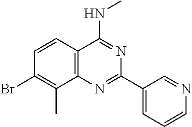 [SMILES: CNc1nc(-c2cccnc2)nc2c(C)c(Br)ccc12] | 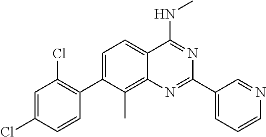 [SMILES: CNc1nc(-c2cccnc2)nc2c(C)c(-c3ccc(Cl)cc3Cl)ccc12] | free | 1 1H NMR (400 MHz, DMSO) δ 9.68 (d, J = 2.1 Hz, 1H), 8.82 (dt, J = 7.9, 2.0, 2.0 Hz, 1H), 8.69 (dd, J = 4.7, 1.8 Hz, 1H), 8.49 (q, J = 4.4, 4.4, 4.3 Hz, 1H), | DMSO | >98 | Method R3 Temper- ature at 80° C. | | | |
| | | | | | 8.14 (d, J = 8.5 | | | | | | |
| | | | | | Hz, 1H), 7.82 (d, | | | | | | |
| | | | | | J = 2.1 Hz, 1H), | | | | | | |
| | | | | | 7.62-7.47 (m, | | | | | | |
| | | | | | 2H), 7.44 (d, J = | | | | | | |
| | | | | | 8.3 Hz, 1H), 7.28 | | | | | | |
| | | | | | (d, J = 8.4 Hz, | | | | | | |
| | | | | | 1H), 3.18 (d, J = | | | | | | |
| | | | | | 4.3 Hz, 3H), 2.44 | | | | | | |
| | | | | | (s, 3H). | | | | | | |
| 929 | 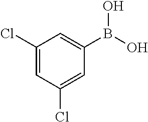 [SMILES: OB(O)c1cc(Cl)cc(Cl)c1] | 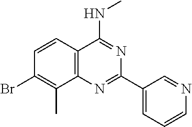 [SMILES: CNc1nc(-c2cccnc2)nc2c(C)c(Br)ccc12] | 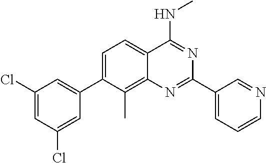 [SMILES: CNc1nc(-c2cccnc2)nc2c(C)c(-c3cc(Cl)cc(Cl)c3)ccc12] | 2HCl | 1H NMR (400 MHz, DMSO) δ 9.73 (d, J = 1.9 Hz, 1H), 9.30 (s, 1H), 8.93 (s, 1H), 8.74 (s, 1H), 8.20 (d, J = 8.5 Hz, 1H), 8.02 (s, 1H), 7.72 (t, J = 1.9, 1.9 Hz, 1H), 7.55 (d, J = 1.9 Hz, 2H), 7.48 (d, | DMSO | >98 | Method R3 Temper- ature at 80° C. | | | |
| | | | | | J = 8.5 Hz, 1H), | | | | | | |
| | | | | | 3.20 (d, J = 4.2 | | | | | | |
| | | | | | Hz, 3H), 2.64 (s, | | | | | | |
| | | | | | 3H). 1H of 2HCl | | | | | | |
| | | | | | was not observed. | | | | | | |
| 930 | 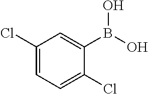 [SMILES: OB(O)c1cc(Cl)ccc1Cl] | 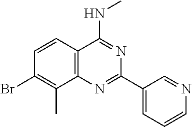 [SMILES: CNc1nc(-c2cccnc2)nc2c(C)c(Br)ccc12] | 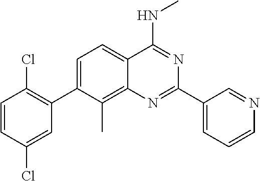 [SMILES: CNc1nc(-c2cccnc2)nc2c(C)c(-c3cc(Cl)ccc3Cl)ccc12] | Free | 1H NMR (100 MHz, DMSO) δ 9.68 (dd, J = 2.2, 0.9 Hz, 1H), 8.82 (dt, J = 8.0, 2.0, 2.0 Hz, 1H), 8.69 (dd, J = 4.8, 1.7 Hz, 1H), 8.50 (d, J = 4.9 Hz, 1H), 8.14 (d, J = 8.5 Hz, 1H), 7.74-7.64 (m, 1H), 7.60-7.49 | DMSO | >98 | Method R3 Temper- ature at 80° C. | | | |
| | | | | | (m, 3H), 7.30 (d, | | | | | | |
| | | | | | J = 8.4 Hz, 1H), | | | | | | |
| | | | | | 3.18 (d, J = 4.4 | | | | | | |
| | | | | | Hz, 3H), 2.45 (s, | | | | | | |
| | | | | | 3H). | | | | | | |
| 931 | 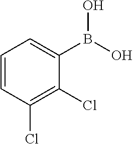 [SMILES: OB(O)c1cccc(Cl)c1Cl] | 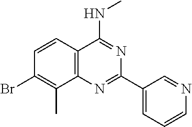 [SMILES: CNc1nc(-c2cccnc2)nc2c(C)c(Br)ccc12] | 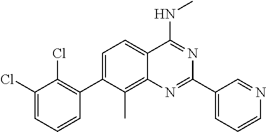 [SMILES: CNc1nc(-c2cccnc2)nc2c(C)c(-c3cccc(Cl)c3Cl)ccc12] | TSA | 1H NMR (400 MHz, DMSO) δ 9.72 (d, J = 2.0 Hz, 1H), 9.15 (s, 1H), 8.86 (d, J = 5.1 Hz, 1H), 8.65 (s, 1H), 8.18 (d, J = 8.5 Hz, 1H), 7.88 (s, 1H), 7.76 (dd, | DMSO | >98 | Method R3 Temper- ature at 80° C. | | | |
| | | | | | J = 8.0, 1.5 Hz, | | | | | | |
| | | | | | 1H), 7.52 (t, J = | | | | | | |
| | | | | | 7.8, 7.8 Hz, 1H), | | | | | | |
| | | | | | 7.49-7.43 (m, | | | | | | |
| | | | | | 2H), 7.39 (dd, J = | | | | | | |
| | | | | | 7.7, 1.5 Hz, 1H), | | | | | | |
| | | | | | 7.36 (d, J = 8.5 | | | | | | |
| | | | | | Hz, 1H), 7.11 (d, | | | | | | |
| | | | | | J = 7.8 Hz, 2H), | | | | | | |
| | | | | | 3.20 (d, J = 4.3 | | | | | | |
| | | | | | Hz, 3H), 2.45 (s, | | | | | | |
| | | | | | 3H), 2.29 (s, 3H). | | | | | | |
| | | | | | 1H of TSA was | | | | | | |
| | | | | | not observed. | | | | | | |
| 932 | 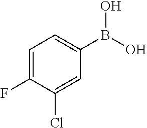 [SMILES: OB(O)c1ccc(F)c(Cl)c1] | 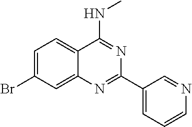 [SMILES: CNc1nc(-c2cccnc2)nc2cc(Br)ccc12] | 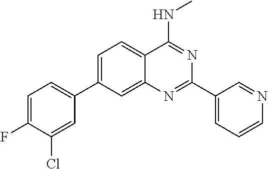 [SMILES: CNc1nc(-c2cccnc2)nc2cc(-c3ccc(F)c(Cl)c3)ccc12] | HCl | 1H NMR (400 MHz, DMSO) δ 9.63 (d, J = 2.1 Hz, 1H), 8.90 (d, J = 8.1 Hz, 1H), 8.84 (dd, J = 4.8, 1.7 H7, 1H), 8.41 (d, J = 8.7 Hz, 1H), 8.22- 8.10 (m, 2H), 8.01 (d, J = 8.1 Hz, 1H), 7.95-7.86 | DMSO | >98 | Method R3 Temper- ature at 80° C. | | | |
| | | | | | (m, 1H), 7.75 (dd, | | | | | | |
| | | | | | J = 7.9, 5.0 Hz, | | | | | | |
| | | | | | 1H), 7.61 (t, J = | | | | | | |
| | | | | | 8.9, 8.9 Hz, 1H), | | | | | | |
| | | | | | 3.24 (d, J = 4.5 | | | | | | |
| | | | | | Hz, 3H). 1H of NH | | | | | | |
| | | | | | and HCl were not | | | | | | |
| | | | | | observed. | | | | | | |
| 933 | 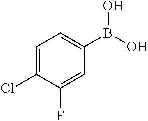 [SMILES: OB(O)c1ccc(Cl)c(F)c1] | 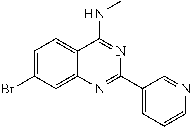 [SMILES: CNc1nc(-c2cccnc2)nc2cc(Br)ccc12] | 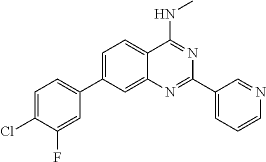 [SMILES: CNc1nc(-c2cccnc2)nc2cc(-c3ccc(Cl)c(F)c3)ccc12] | 2HCl | 1H NMR (400 MHz, DMSO) δ 9.63 (d, J = 2.3 Hz, 2H), 9.00 (s, 1H), 8.92 (d, J = 5.0 Hz, 1H), 8.49 (d, J = 8.7 Hz, 1H), 8.27 (s, 1H), 8.15- 8.04 (m, 1H), 7.99 (dd, J = 10.8, 2.0 Hz, 1H), 7.87 | DMSO | >98 | Method R3 Temper- ature at 80° C. | | | |
| | | | | | (dd, J = 8.1, 4.9 | | | | | | |
| | | | | | Hz, 1H), 7.83-7.70 | | | | | | |
| | | | | | (m, 2H), 3.27 | | | | | | |
| | | | | | (d, J = 4.5 Hz, 3H). | | | | | | |
| | | | | | 1H of 2HCl was | | | | | | |
| | | | | | not observed. | | | | | | |
| 934 | 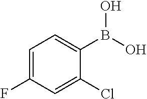 [SMILES: OB(O)c1ccc(F)cc1Cl] | 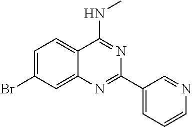 [SMILES: CNc1nc(-c2cccnc2)nc2cc(Br)ccc12] | 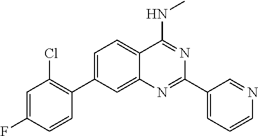 [SMILES: CNc1nc(-c2cccnc2)nc2cc(-c3ccc(F)cc3Cl)ccc12] | HCl | 1H NMR (400 MHz, DMSO) δ 9.63 (d, J = 2.3 Hz, 1H), 8.99 (s, 1H), 8.93 (dd, J = 4.8, 1.7 Hz, 1H), 8.51 (d, J = 8.4 Hz, 1H), 8.06 (s, 1H), 7.91-7.81 (m, | DMSO | >98 | Method R3 Temper- ature at 80° C. | | | |
| | | | | | 1H), 7.78 (d, J = | | | | | | |
| | | | | | 8.6 Hz, 1H), 7.70 | | | | | | |
| | | | | | (dd, J = 8.9, 2.6 | | | | | | |
| | | | | | Hz, 1H), 7.64 (dd, | | | | | | |
| | | | | | J = 8.6, 6.1 Hz, | | | | | | |
| | | | | | 1H), 7.43 (td, J = | | | | | | |
| | | | | | 8.5, 8.5, 2.6 Hz, | | | | | | |
| | | | | | 1H), 3.29 (d, J = | | | | | | |
| | | | | | 4.4 Hz, 3H). 1H | | | | | | |
| | | | | | of HCl and NH were | | | | | | |
| | | | | | not observed. | | | | | | |
| 935 | 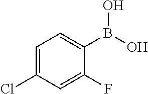 [SMILES: OB(O)c1ccc(Cl)cc1F] | 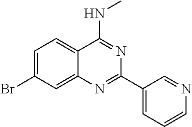 [SMILES: CNc1nc(-c2cccnc2)nc2cc(Br)ccc12] | 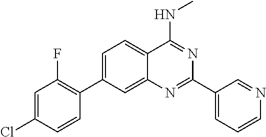 [SMILES: CNc1nc(-c2cccnc2)nc2cc(-c3ccc(Cl)cc3F)ccc12] | 2HCl | 1H NMR (400 MHz, DMSO) δ 10.19- 9.73 (m, 1H), 9.71- 9.47 (m, 1H), 9.02 (s, 1H), 8.95 (dd, J = 5.1, 1.6 Hz, 1H), 8.56 (d, J = 8.7 Hz, 1H), 8.24 (s, 1H), 8.00- | DMSO | >98 | Method R3 Temper- ature at 80° C. | | | |
| | | | | | 7.82 (m, 2H), | | | | | | |
| | | | | | 7.77 (t, J = 8.5, 8.5 | | | | | | |
| | | | | | Hz, 1H), 7.70 | | | | | | |
| | | | | | (dd, J = 10.8, 2.1 | | | | | | |
| | | | | | Hz, 1H), 7.52 (dd, | | | | | | |
| | | | | | J = 8.3, 2.0 Hz, 1H), | | | | | | |
| | | | | | 3.29 (d, J = 4.4 | | | | | | |
| | | | | | Hz, 3H). 1H of | | | | | | |
| | | | | | 2HCl was not | | | | | | |
| | | | | | observed. | | | | | | |
| 936 | 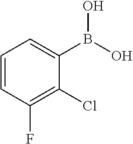 [SMILES: OB(O)c1cccc(F)c1Cl] | 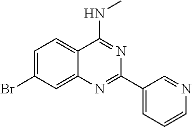 [SMILES: CNc1nc(-c2cccnc2)nc2cc(Br)ccc12] | 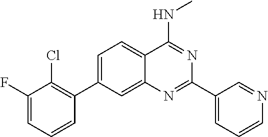 [SMILES: CNc1nc(-c2cccnc2)nc2cc(-c3cccc(F)c3Cl)ccc12] | 2HCl | 1H NMR (400 MHz, DMSO) δ 9.64 (d, J = 2.1 Hz, 1H), 9.05 (s, 1H), 8.95 (dd, J = 5.1, 1.6 Hz, 1H), 8.55 (d, J = 8.5 Hz, 1H), 8.14 (s, 1H), 7.89 (s, 1H), 7.82 | DMSO | >98 | Method R3 Temper- ature at 80° C. | | | |
| | | | | | (d, J = 8.2 Hz, | | | | | | |
| | | | | | 1H), 7.65-7.51 | | | | | | |
| | | | | | (m, 2H), 7.51- | | | | | | |
| | | | | | 7.31 (m, 1H), 3.30 | | | | | | |
| | | | | | (d, J = 4.4 Hz, | | | | | | |
| | | | | | 3H). 1H of 2HCl | | | | | | |
| | | | | | and NH were not | | | | | | |
| | | | | | observed. | | | | | | |
| 937 | 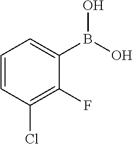 [SMILES: OB(O)c1cccc(Cl)c1F] | 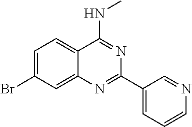 [SMILES: CNc1nc(-c2cccnc2)nc2cc(Br)ccc12] | 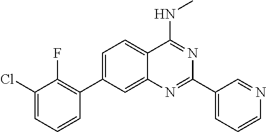 [SMILES: CNc1nc(-c2cccnc2)nc2cc(-c3cccc(Cl)c3F)ccc12] | 2HCl | 1H NMR (400 MHz, DMSO) δ 9.63 (d, J = 2.1 Hz, 1H), 9.00 (s, 1H), 8.92 (dd, J = 5.1, 1.6 Hz, 1H), 8.51 (d, J = 8.5 Hz, 1H), 8.17 (s, 1H), 7.97-7.81 (m, 2 | DMSO | >98 | Method R3 Temper- ature at 80° C. | | | |
| | | | | | H), 7.81-7.60 | | | | | | |
| | | | | | (m, 2H), 7.44 (td, | | | | | | |
| | | | | | J = 8.0, 8.0, 1.0 | | | | | | |
| | | | | | Hz, 1H), 3.28 (d, | | | | | | |
| | | | | | J = 4.4 Hz, 3H). | | | | | | |
| | | | | | 1H of 2HCl and | | | | | | |
| | | | | | NH were not | | | | | | |
| | | | | | observed. | | | | | | |
| 938 | 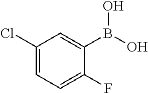 [SMILES: OB(O)c1cc(Cl)ccc1F] | 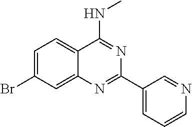 [SMILES: CNc1nc(-c2cccnc2)nc2cc(Br)ccc12] | 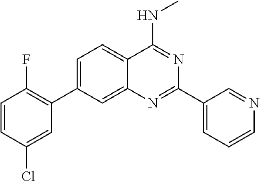 [SMILES: CNc1nc(-c2cccnc2)nc2cc(-c3cc(Cl)ccc3F)ccc12] | 2HCl | 1H NMR (400 MHz, DMSO) δ 9.63 (d, J = 2.1 Hz, 1H), 9.01 (s, 1H), 8.94 (dd, J = 5.1, 1.6 Hz, 1H), 8.53 (d, J = 8.5 Hz, 1H), 8.21 (s, 1H), 7.94 (d, J = 8.5 Hz, 1H), 7.88 (s, 1H), 7.83 (dd, J = 6.8, 2.7 Hz, 1H), | DMSO | >98 | Method R3 Temper- ature at 80° C. | | | |
| | | | | | 7.63 (ddd, J = 8.8, | | | | | | |
| | | | | | 4.2, 2.7 Hz, 1H), | | | | | | |
| | | | | | 7.51 (dd, J = 10.4, | | | | | | |
| | | | | | 8.8 Hz, 1H), | | | | | | |
| | | | | | 3.29 (d, J = 4.4 | | | | | | |
| | | | | | Hz, 3H). | | | | | | |
| | | | | | 1H of 2HCl and | | | | | | |
| | | | | | NH were not | | | | | | |
| | | | | | observed. | | | | | | |
| 939 | 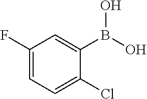 [SMILES: OB(O)c1cc(F)ccc1Cl] | 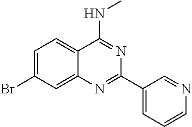 [SMILES: CNc1nc(-c2cccnc2)nc2cc(Br)ccc12] | 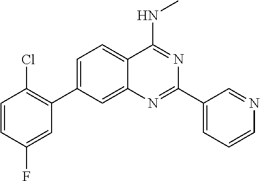 [SMILES: CNc1nc(-c2cccnc2)nc2cc(-c3cc(F)ccc3Cl)ccc12] | 2HCl | 1H NMR (400 MHz, DMSO) δ 9.70- 9.54 (m, 1H), 9.05 (s, 1H), 8.95 (dd, J = 5.1, 1.6 Hz, 1H), 8.55 (d, J = 8.5 Hz, 1H), 8.14 (s, 1H), 7.90 (t, J = 6.7, 6.7 Hz, 1H), 7.86-7.77 (m, 1H), 7.73 (dd, J = 8.9, 5.1 Hz, 1H), | DMSO | >98 | Method R3 Temper- ature at 80° C. | | | |
| | | | | | 7.52 (dd, J = 9.1, | | | | | | |
| | | | | | 3.1 Hz, 1H), 7.48- | | | | | | |
| | | | | | 7.29 (m, 1H), | | | | | | |
| | | | | | 3.29 (d, J = 4.5 Hz, | | | | | | |
| | | | | | 3H). 1H of 2HCl and | | | | | | |
| | | | | | NH were not | | | | | | |
| | | | | | observed. | | | | | | |
| 940 | 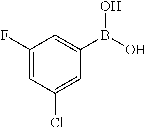 [SMILES: OB(O)c1cc(F)cc(Cl)c1] | 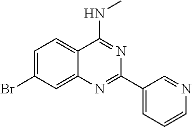 [SMILES: CNc1nc(-c2cccnc2)nc2cc(Br)ccc12] | 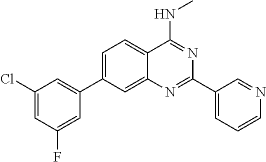 [SMILES: CNc1nc(-c2cccnc2)nc2cc(-c3cc(F)cc(Cl)c3)ccc12] | 2HCl | 1H NMR (400 MHz, DMSO) δ 9.64 (d, J = 2.1 Hz, 1H), 9.02 (s, 1H), 8.92 (dd, J = 5.1, 1.6 Hz, 1H), 8.48 (d, J = 8.6 Hz, 1H), 8.27 (s, 1H), 8.10 (d, J = 8.6 Hz, 1H), 7.88 (s, 1H), 7.84 (t, J = | DMSO | >98 | Method R3 Temper- ature at 80° C. | | | |
| | | | | | 1.7, 1.7 Hz, 1H), | | | | | | |
| | | | | | 7.78 (d, J = 9.7 | | | | | | |
| | | | | | Hz, 1H), 7.60 (d, | | | | | | |
| | | | | | J = 8.6 Hz, 1H), 3.27 | | | | | | |
| | | | | | (d, J = 4.4 Hz, | | | | | | |
| | | | | | 3H). 1H of 2HCl | | | | | | |
| | | | | | and NH were not | | | | | | |
| | | | | | observed. | | | | | | |
| 941 | 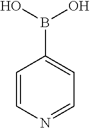 | 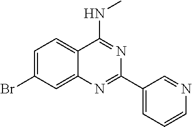 [SMILES: CNc1nc(-c2cccnc2)nc2cc(Br)ccc12] | 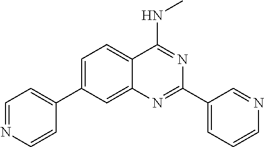 [SMILES: CNc1nc(-c2cccnc2)nc2cc(-c3ccncc3)ccc12] | | 1H NMR (400 MHz, DMSO) δ 9.66 (d, J = 2.2 Hz, 1H), 9.01 (s, 1H), 8.96-8.77 (m, 3H), 8.53 (d, J = 8.7 Hz, 1H), 8.42 (d, J = 1.9 Hz, 1H), 8.25 (s, 2H), 8.17 (d, J = 8.6 | DMSO | >98 | Method R3 Temper- ature at 80° C. | | | |
| | | | | | Hz, 1H), 7.85 (d, | | | | | | |
| | | | | | J = 6.9 Hz, 1H), | | | | | | |
| | | | | | 3.25 (d, J = 4.4 Hz, | | | | | | |
| | | | | | 3H). 1H of NH | | | | | | |
| | | | | | was not observed. | | | | | | |
| 942 | 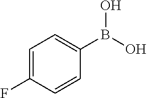 | 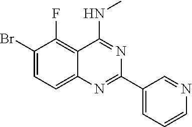 [SMILES: CNc1nc(-c2cccnc2)nc2ccc(Br)c(F)c12] | 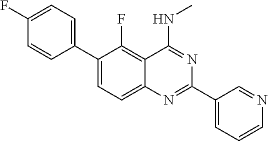 [SMILES: CNc1nc(-c2cccnc2)nc2ccc(-c3ccc(F)cc3)c(F)c12] | 2HCl | 1H NMR (400 MHz, DMSO) δ 9.67 (d, J = 2.1 Hz, 1H), 9.16 (d, J = 8.1 Hz, 1H), 8.94 (d, J = 5.1 Hz, 1H), 8.55 (s, 1H), 8.08- 7.91 (m, 2H), 7.83 (d, J = 8.6 Hz, | DMSO | >98 | Method R3 Temper- ature at 80° C. | | | |
| | | | | | 1H), 7.72 (dd, | | | | | | |
| | | | | | J = 8.2, 5.4 Hz, 2H), | | | | | | |
| | | | | | 7.51-7.34 (m, | | | | | | |
| | | | | | 2H), 3.24 (d, J = 4.4 | | | | | | |
| | | | | | Hz, 3H). 1H of | | | | | | |
| | | | | | 2HC□ was not | | | | | | |
| | | | | | observed. | | | | | | |
| 943 | 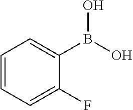 | 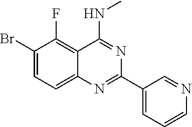 [SMILES: CNc1nc(-c2cccnc2)nc2ccc(Br)c(F)c12] | 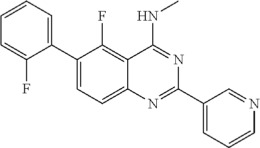 [SMILES: CNc1nc(-c2cccnc2)nc2ccc(-c3ccccc3F)c(F)c12] | 2HCl | 1H NMR (400 MHz, DMSO) δ 9.67 (d, J = 2.0 Hz, 1H), 9.15 (d, J = 8.2 Hz, 1H), 8.94 (dd, J = 5.1, 1.5 Hz, 1H), 8.51 (s, 1H), 8.04-7.88 (m, 2H), 7.83 (d, J = 8.6 Hz, 1H), 7.65- | DMSO | >98 | Method R3 Temper- ature at 80° C. | | | |
| | | | | | 7.53 (m, 2H), 7.41 | | | | | | |
| | | | | | (q, J = 8.0, 8.0, | | | | | | |
| | | | | | 7.2 Hz, 2H), 3.23 | | | | | | |
| | | | | | (d, J = 4.4 Hz, | | | | | | |
| | | | | | 3H). 1H of 2HC□ | | | | | | |
| | | | | | was not | | | | | | |
| | | | | | observed. | | | | | | |
| 944 | 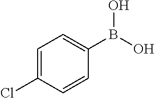 | 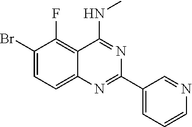 [SMILES: CNc1nc(-c2cccnc2)nc2ccc(Br)c(F)c12] | 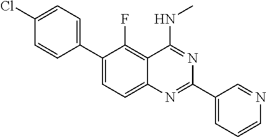 [SMILES: CNc1nc(-c2cccnc2)nc2ccc(-c3ccc(Cl)cc3)c(F)c12] | 2HCl | 1H NMR (400 MHz, DMSO) δ 9.66 (d, J = 2.0 Hz, 1H), 9.10 (d, J = 8.1 Hz, 1H), 8.91 (dd, J = 5.2, 1.7 Hz, 1H), 8.47 (s, 1H), 8.00 (t, J = 8.5, 8.5 Hz, 1H), 7.91 | DMSO | >98 | Method R3 Temper- ature at 80° C. | | | |
| | | | | | (dd, J = 8.1, 5.1 | | | | | | |
| | | | | | Hz, 1H), 7.80 (d, | | | | | | |
| | | | | | J = 8.7 Hz, 1H), | | | | | | |
| | | | | | 7.76-7.67 (m, | | | | | | |
| | | | | | 2H), 7.67-7.57 | | | | | | |
| | | | | | (m, 2H), 3.23 (d, | | | | | | |
| | | | | | J = 4.3 Hz, 3H). | | | | | | |
| | | | | | 1H of 2HC□ was | | | | | | |
| | | | | | not observed. | | | | | | |
| 945 | 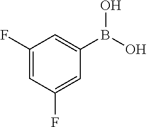 [SMILES: OB(O)c1cc(F)cc(F)c1] | 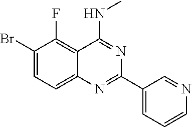 [SMILES: CNc1nc(-c2cccnc2)nc2ccc(Br)c(F)c12] | 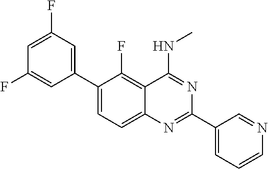 [SMILES: CNc1nc(-c2cccnc2)nc2ccc(-c3cc(F)cc(F)c3)c(F)c12] | 3HCl | 1H NMR (400 MHz, DMSO) δ 9.67 (d, J = 2.1 Hz, 1H), 9.20 (d, J = 8.0 Hz, 1H), 8.96 (d, J = 5.2 Hz, 1H), 8.54 (s, 1H), 8.07 (t, J = 8.5, 8.5 Hz, 1H), 7.99 (dd, J = 8.1, 5.2 Hz, | DMSO | >98 | Method R3 | | | |
| | | | | | 1H), 7.83 (d, J = | | | | | | |
| | | | | | 8.7 Hz, 1H), 7.57- | | | | | | |
| | | | | | 7.25 (m, 3H), 3.24 | | | | | | |
| | | | | | (d, J = 4.4 Hz, | | | | | | |
| | | | | | 3H). 1H of 3HC□ | | | | | | |
| | | | | | was not | | | | | | |
| | | | | | observed. | | | | | | |
| 946 | 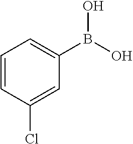 | 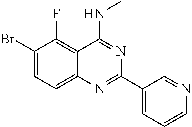 [SMILES: CNc1nc(-c2cccnc2)nc2ccc(Br)c(F)c12] | 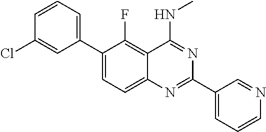 [SMILES: CNc1nc(-c2cccnc2)nc2ccc(-c3cccc(Cl)c3)c(F)c12] | HCl | 1H NMR (400 MHz, DMSO) δ 9.67 (d, J = 2.0 Hz, 1H), 9.12 (d, J = 8.1 Hz, 1H), 8.92 (dd, J = 5.2, 1.6 Hz, 1H), 8.49 (s, 1H), 8.04 (t, J = 8.5, 8.5 Hz, 1H), 7.92 | DMSO | >98 | Method R3 Temper- ature at 80° C. | | | |
| | | | | | (dd, J = 7.9, 5.1 | | | | | | |
| | | | | | Hz, 1H), 7.85-7.72 | | | | | | |
| | | | | | (m, 2H), 7.72- | | | | | | |
| | | | | | 7.39 (m, 3H), 3.23 | | | | | | |
| | | | | | (d, J = 4.4 Hz, | | | | | | |
| | | | | | 3H). 1H of HC□ | | | | | | |
| | | | | | was not observed. | | | | | | |
| 947 | 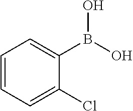 | 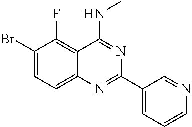 [SMILES: CNc1nc(-c2cccnc2)nc2ccc(Br)c(F)c12] | 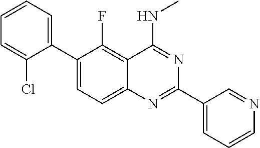 [SMILES: CNc1nc(-c2cccnc2)nc2ccc(-c3ccccc3Cl)c(F)c12] | 2HCl | 1H NMR (400 MHz, DMSO) δ 9.67 (d, J = 2.0 Hz, 1H), 9.25-9.03 (m, 1H), 8.93 (dd, J = 5.2, 1.6 Hz, 1H), 8.46 (s, 1H), 7.93 (dd, J = 8.0, 5.2 Hz, 1H), 7.90-7.76 (m, 2H), 7.68 | DMSO | >98 | Method R3 Temper- ature at 80° C. | | | |
| | | | | | (dd, J = 7.3, 2.0 | | | | | | |
| | | | | | Hz, 1H), 7.61- | | | | | | |
| | | | | | 7.45 (m, 3H), 3.22 | | | | | | |
| | | | | | (d, J = 4.4 Hz, | | | | | | |
| | | | | | 3H). 1H of 2HC□ | | | | | | |
| | | | | | was not | | | | | | |
| | | | | | observed. | | | | | | |
| 948 | 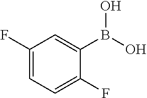 [SMILES: OB(O)c1cc(F)ccc1F] | 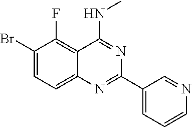 [SMILES: CNc1nc(-c2cccnc2)nc2ccc(Br)c(F)c12] | 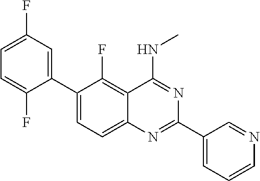 [SMILES: CNc1nc(-c2cccnc2)nc2ccc(-c3cc(F)ccc3F)c(F)c12] | 2HCl | 1H NMR (400 MHz, DMSO) δ 9.66 (d, J = 2.1 Hz, 1H), 9.07 (d, J = 9.0 Hz, 1H), 8.89 (dd, J = 5.0, 1.6 Hz, 1H), 8.38 (s, 1H), 8.02-7.84 (m, 2H), 7.79 (d, J = 8.6 Hz, 1H), 7.60- 7.31 (m, 3H), 3.22 (d, J = 4.5 Hz, | DMSO | >98 | Method R3 | | | |
| | | | | | 3H). 1H of 2HC□ | | | | | | |
| | | | | | was not | | | | | | |
| | | | | | observed. | | | | | | |
| 949 | 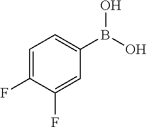 [SMILES: OB(O)c1ccc(F)c(F)c1] | 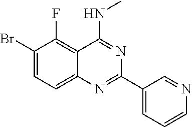 [SMILES: CNc1nc(-c2cccnc2)nc2ccc(Br)c(F)c12] | 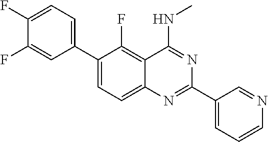 [SMILES: CNc1nc(-c2cccnc2)nc2ccc(-c3ccc(F)c(F)c3)c(F)c12] | 2HCl | 1H NMR (400 MHz, DMSO) δ 9.67 (d, J = 2.0 Hz, 1H), 9.17 (d, J = 8.1 Hz, 1H), 8.94 (dd, J = 5.2, 1.6 Hz, 1H), 8.52 (s, 1H), 8.03 (t, J = 8.5, 8.5 Hz, 1H), 7.97 (dd, J = 8.1, 5.3 | DMSO | >98 | Method R3 | | | |
| | | | | | Hz, 1H), 7.90-7.74 | | | | | | |
| | | | | | (m, 2H), 7.65 | | | | | | |
| | | | | | (dt, J = 10.7, 8.5, 8.5 | | | | | | |
| | | | | | Hz, 1H), 7.54 | | | | | | |
| | | | | | (s, 1H), 3.23 (d, J = | | | | | | |
| | | | | | 4.4 Hz, 3H). 1H | | | | | | |
| | | | | | of 2HC□ was | | | | | | |
| | | | | | not observed. | | | | | | |
| 950 | 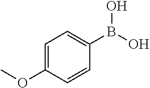 | 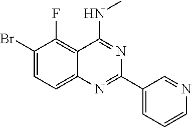 [SMILES: CNc1nc(-c2cccnc2)nc2ccc(Br)c(F)c12] | 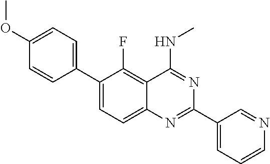 [SMILES: CNc1nc(-c2cccnc2)nc2ccc(-c3ccc(OC)cc3)c(F)c12] | 2HCl | 1H NMR (400 MHz, DMSO) δ 9.65 (d, J = 2.1 Hz, 1H), 9.05 (d, J = 8.3 Hz, 1H), 8.89 (dd, J = 5.1, 1.6 Hz, 1H), 8.46 (s, 1H), 7.98 (t, J = 8.6, 8.6 Hz, 1H), 7.92- 7.83 (m, 1H), 7.77 (d, J = 8.7 Hz, | DMSO | >98 | Method R3 | | | |
| | | | | | 1H), 7.61 (dd, | | | | | | |
| | | | | | J = 8.7, 1.6 Hz, 2H), | | | | | | |
| | | | | | 7.26-6.93 (m, | | | | | | |
| | | | | | 2H), 3.84 (s, 3H), | | | | | | |
| | | | | | 3.23 (d, J = 4.5 | | | | | | |
| | | | | | Hz, 3H). 1H of | | | | | | |
| | | | | | 2HC□ was not | | | | | | |
| | | | | | observed. | | | | | | |
| 951 | 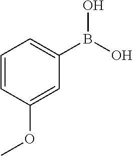 | 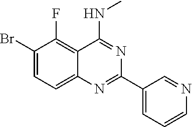 [SMILES: CNc1nc(-c2cccnc2)nc2ccc(Br)c(F)c12] | 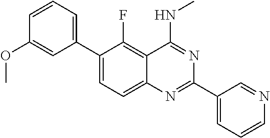 [SMILES: CNc1nc(-c2cccnc2)nc2ccc(-c3cccc(OC)c3)c(F)c12] | 2HCl | 1H NMR (400 MHz, DMSO) δ 9.67 (d, J = 2.0 Hz, 1H), 9.14 (d, J = 7.9 Hz, 1H), 8.94 (d, J = 5.2 Hz, 1H), 8.55 (s, 1H), 8.03 (t, J = 8.4, 8.4 Hz, 1H), 7.94 (dd, | DMSO | >98 | Method R3 | | | |
| | | | | | J = 8.0, 5.2 Hz, | | | | | | |
| | | | | | 1H), 7.82 (d, J = | | | | | | |
| | | | | | 8.7 Hz, 1H), 7.47 | | | | | | |
| | | | | | (t, J = 7.9, 7.9 Hz, | | | | | | |
| | | | | | 1H), 7.31-7.11 | | | | | | |
| | | | | | (m, 2H), 7.06 (dd, | | | | | | |
| | | | | | J = 8.3, 2.4 Hz, | | | | | | |
| | | | | | 1H), 3.84 (s, 3H), | | | | | | |
| | | | | | 3.24 (d, J = 4.4 | | | | | | |
| | | | | | Hz, 3H). 1H of | | | | | | |
| | | | | | 2HC□ was not | | | | | | |
| | | | | | observed. | | | | | | |
| 952 | 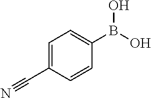 [SMILES: N#Cc1ccc(B(O)O)cc1] | 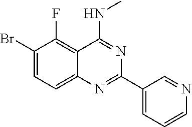 [SMILES: CNc1nc(-c2cccnc2)nc2ccc(Br)c(F)c12] | 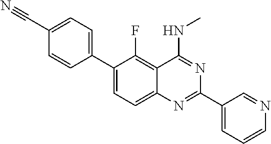 [SMILES: CNc1nc(-c2cccnc2)nc2ccc(-c3ccc(C#N)cc3)c(F)c12] | 2HCl | 1H NMR (400 MHz, DMSO) δ 9.67 (d, J = 2.0 Hz, 1H), 9.12 (d, J = 7.6 Hz, 1H), 8.92 (dd, J = 5.2, 1.6 Hz, 1H), 8.46 (d, J = 10.1 Hz, 1H), 8.04 (dd, J = 8.4, 2.1 Hz, 3H), 7.96- | DMSO | >98 | Method R3 | | | |
| | | | | | 7.85 (m, 3H), 7.82 | | | | | | |
| | | | | | (d, J = 8.7 Hz, | | | | | | |
| | | | | | 1H), 3.23 (d, J = 4.4 | | | | | | |
| | | | | | Hz, 3H). 1H of | | | | | | |
| | | | | | 2HC□ was not | | | | | | |
| | | | | | observed. | | | | | | |
| 953 | 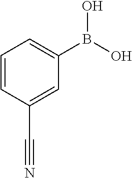 [SMILES: N#Cc1cccc(B(O)O)c1] | 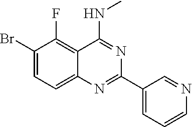 [SMILES: CNc1nc(-c2cccnc2)nc2ccc(Br)c(F)c12] | 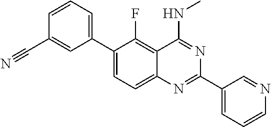 [SMILES: CNc1nc(-c2cccnc2)nc2ccc(-c3cccc(C#N)c3)c(F)c12] | 2HCl | 1H NMR (400 MHz, DMSO) 5 9.67 (d, J = 2.0 Hz, 1H), 9.17 (d, J = 7.9 Hz, 1H), 8.94 (d, J = 5.2 Hz, 1H), 8.52 (t, J = 8.2, 8.2 Hz, 1H), 8.17 (s, 1H), 8.06 (dd, J = 17.0, 8.6 Hz, 1H), 8.03-7.92 | DMSO | >98 | Method R3 | | | |
| | | | | | (m, 3H), 7.91- | | | | | | |
| | | | | | 7.62 (m, 2H), 3.24 | | | | | | |
| | | | | | (d, J = 4.4 Hz, | | | | | | |
| | | | | | 3H). 1H of 2HC□ | | | | | | |
| | | | | | was not observed | | | | | | |
| 954 | 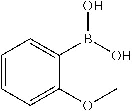 | 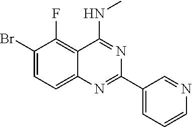 [SMILES: CNc1nc(-c2cccnc2)nc2ccc(Br)c(F)c12] | 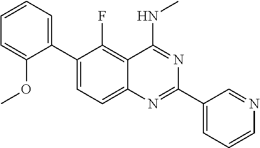 [SMILES: CNc1nc(-c2cccnc2)nc2ccc(-c3ccccc3OC)c(F)c12] | 2HCl | 1H NMR (400 MHz, DMSO) δ 9.65 (d, J = 2.0 Hz, 1H), 9.12 (3rd, 1H), 8.94 (d, J = 5.2 Hz, 1H), 8.57 (s, 1H), 7.95(d, J = 5.3 Hz, 1H), 7.90- 7.81 (m, 1H), 7.83- 7.70 (m, 1H), | DMSO | >98 | Method R3 | | | |
| | | | | | 7.49 (d, J = 1.7 Hz, | | | | | | |
| | | | | | 1H), 7.36 (dd, | | | | | | |
| | | | | | J = 7.6, 1.7 Hz, 1H), | | | | | | |
| | | | | | 7.21 (d, J = 8.4 | | | | | | |
| | | | | | Hz, 1H), 7.11 (t, | | | | | | |
| | | | | | J = 7.4, 7.4 Hz, | | | | | | |
| | | | | | 1H), 3.78 (s, 3H), | | | | | | |
| | | | | | 3.23 (d, J = 4.4 | | | | | | |
| | | | | | Hz, 3H). 1H of | | | | | | |
| | | | | | 2HC□ was not | | | | | | |
| | | | | | observed. | | | | | | |
| 955 | 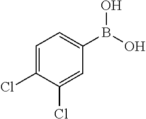 [SMILES: OB(O)c1ccc(Cl)c(Cl)c1] | 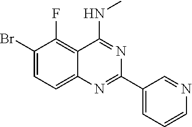 [SMILES: CNc1nc(-c2cccnc2)nc2ccc(Br)c(F)c12] | 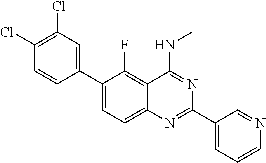 [SMILES: CNc1nc(-c2cccnc2)nc2ccc(-c3ccc(Cl)c(Cl)c3)c(F)c12] | 2HCl | 1H NMR (400 MHz, DMSO) δ 9.68 (d, J = 2.0 Hz, 1H), 9.22 (d, J = 8.0 Hz, 1H), 8.97 (dd, J = 5.3, 1.6 Hz, 1H), 8.58 (s, 1H), 8.06 (t, J = 8.5, 8.5 Hz, 1H), 8.03- 7.93 (m, 2H), 7.91-7.79 (m, | DMSO | >98 | Method R3 Temper- ature at 80° C. | | | |
| | | | | | 2H), 7.73-7.63 | | | | | | |
| | | | | | (m, 1H), 3.24 (d, | | | | | | |
| | | | | | J = 4.4 Hz, 3H). | | | | | | |
| | | | | | 1H of 2HC□ was | | | | | | |
| | | | | | not observed. | | | | | | |
| 956 | 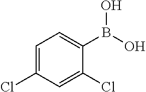 [SMILES: OB(O)c1ccc(Cl)cc1Cl] | 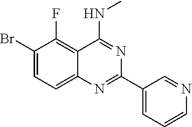 [SMILES: CNc1nc(-c2cccnc2)nc2ccc(Br)c(F)c12] | 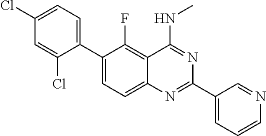 [SMILES: CNc1nc(-c2cccnc2)nc2ccc(-c3ccc(Cl)cc3Cl)c(F)c12] | 2HCl | 1H NMR (400 MHz, DMSO) δ 9.67 (d, J = 2.0 Hz, 1H), 9.17 (d, J = 7.9 Hz, 1H), 8.95 (dd, J = 5.3, 1.6 Hz, 1H), 8.47 (s, 1H), 7.97 (dd, J = 8.1, 5.3 Hz, 1H), 7.93- | DMSO | >98 | Method R3 Temper- ature at 80° C. | | | |
| | | | | | 7.76 (m, 3H), | | | | | | |
| | | | | | 7.70-7.38 (m, 2H), | | | | | | |
| | | | | | 3.22 (d, J = | | | | | | |
| | | | | | 4.4 Hz, 3H). 1H of | | | | | | |
| | | | | | 2HC□ was not | | | | | | |
| | | | | | observed. | | | | | | |
| 957 | 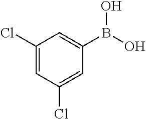 [SMILES: OB(O)c1cc(Cl)cc(Cl)c1] | 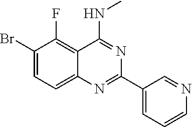 [SMILES: CNc1nc(-c2cccnc2)nc2ccc(Br)c(F)c12] | 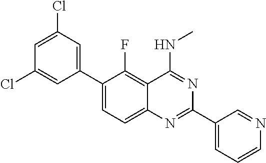 [SMILES: CNc1nc(-c2cccnc2)nc2ccc(-c3cc(Cl)cc(Cl)c3)c(F)c12] | 2HCl | 1H NMR (400 MHz, DMSO) δ 9.67 (dd, J = 2.1, 0.8 Hz, 1H), 9.15 (d, J = 8.1 Hz, 1H), 8.93 (dd, J = 5.3, 1.6 Hz, 1H), 8.50 (s, 1H), 8.07 (t, J = 8.5, 8.5 Hz, 1H), 7.94 (dd, J = 8.0, 5.2 Hz, 1H), 7.88- | DMSO | >98 | Method R3 Temper- ature at 80° C. | | | |
| | | | | | 7.65 (m, 4H), | | | | | | |
| | | | | | 3.23 (d, J = 4.4 | | | | | | |
| | | | | | Hz, 3H). 1H of | | | | | | |
| | | | | | 2HC□ was not | | | | | | |
| | | | | | observed. | | | | | | |
| 958 | 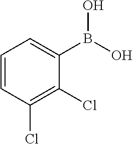 [SMILES: OB(O)c1cccc(Cl)c1Cl] | 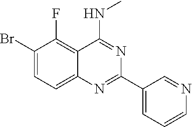 [SMILES: CNc1nc(-c2cccnc2)nc2ccc(Br)c(F)c12] | 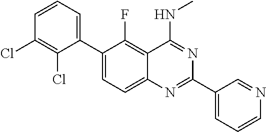 [SMILES: CNc1nc(-c2cccnc2)nc2ccc(-c3cccc(Cl)c3Cl)c(F)c12] | 2HCl | 1H NMR (400 MHz, DMSO) δ 9.68 (d, J = 1.9 Hz, 1H), 9.19 (brd, 1H), 8.95 (d, J = 5.1 Hz, 1H), 8.48 (s, 1H), 7.97 (s, 1H), 7.95-7.70 (m, 3H), 7.66-7.45 (m, | DMSO | >98 | Method R3 Temper- ature at 80° C. | | | |
| | | | | | 2H), 3.22 (d, | | | | | | |
| | | | | | J = 4.4 Hz, 3H). 1H | | | | | | |
| | | | | | of 2HC□ was | | | | | | |
| | | | | | not observed. | | | | | | |
| 959 | 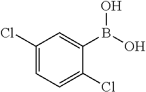 [SMILES: OB(O)c1cc(Cl)ccc1Cl] | 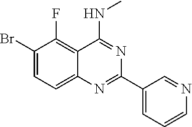 [SMILES: CNc1nc(-c2cccnc2)nc2ccc(Br)c(F)c12] | 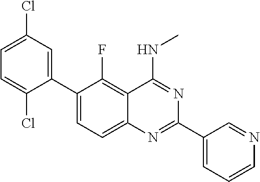 [SMILES: CNc1nc(-c2cccnc2)nc2ccc(-c3cc(Cl)ccc3Cl)c(F)c12] | 2HCl | 1H NMR (400 MHz, DMSO) δ 9.68 (d, J = 2.0 Hz, 1H), 9.19 (d, J = 8.0 Hz, 1H), 8.96 (dd, J = 5.4, 1.6 Hz, 1H), 8.49 (s, 1H), 7.98 (dd, J = 8.0, 5.3 Hz, 1H), 7.94- 7.85 (m, 1H), 7.86-7.79 (m, 1H), 7.76-7.69 | DMSO | >98 | Method R3 Temper- ature at 80° C. | | | |
| | | | | | (m, 2H), 7.67-7.57 | | | | | | |
| | | | | | (m, 1H), 3.22 | | | | | | |
| | | | | | (d, J = 4.4 Hz, 3H). | | | | | | |
| | | | | | 1H of 2HC□ | | | | | | |
| | | | | | was not observed. | | | | | | |
| 960 | 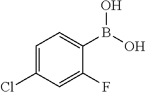 [SMILES: OB(O)c1ccc(Cl)cc1F] | 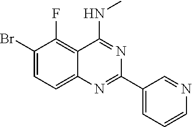 [SMILES: CNc1nc(-c2cccnc2)nc2ccc(Br)c(F)c12] | 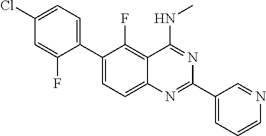 [SMILES: CNc1nc(-c2cccnc2)nc2ccc(-c3ccc(Cl)cc3F)c(F)c12] | 2HCl | 1H NMR (400 MHz, DMSO) δ 9.67 (dd, J = 2.0, 0.8 Hz, 1H), 9.14 (d, J = 8.1 Hz, 1H), 8.93 (dd, J = 5.2, 1.6 Hz, 1H), 8.46 (s, 1H), 7.93 (dt, J = 8.7, 4.8, 4.8 Hz, | DMSO | >98 | Method R3 Temper- ature at 80° C. | | | |
| | | | | | 2H), 7.82 (d, J = | | | | | | |
| | | | | | 8.6 Hz, 1H), 7.77- | | | | | | |
| | | | | | 7.60 (m, 2H), | | | | | | |
| | | | | | 7.60-7.38 (m, 1H), | | | | | | |
| | | | | | 3.22 (d, J = | | | | | | |
| | | | | | 4.4 Hz, 3H). 1H of | | | | | | |
| | | | | | 2HC□ was not | | | | | | |
| | | | | | observed. | | | | | | |
| 961 | 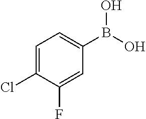 [SMILES: OB(O)c1ccc(Cl)c(F)c1] | 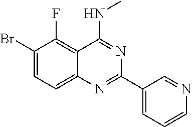 [SMILES: CNc1nc(-c2cccnc2)nc2ccc(Br)c(F)c12] | 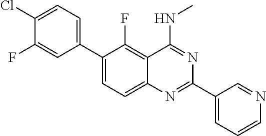 [SMILES: CNc1nc(-c2cccnc2)nc2ccc(-c3ccc(Cl)c(F)c3)c(F)c12] | 2HCl | 1H NMR (400 MHz, DMSO) δ 9.67 (d, J = 2.1 Hz, 1H), 9.16 (d, J = 8.0 Hz, 1H), 8.94 (d, J = 5.0 Hz, 1H), 8.50 (s, 1H), 8.04 (t, J = 8.5, 8.5 Hz, 1H), 7.95 (dd, J = | DMSO | >98 | Method R3 Temper- ature at 80° C. | | | |
| | | | | | 8.1, 5.2 Hz, | | | | | | |
| | | | | | 1H), 7.88-7.68 (m, | | | | | | |
| | | | | | 3H), 7.56 (d, | | | | | | |
| | | | | | J = 8.3 Hz, 1H), 3.23 | | | | | | |
| | | | | | (d, J = 4.4 Hz, | | | | | | |
| | | | | | 3H). 1H of 2HC□ | | | | | | |
| | | | | | was not | | | | | | |
| | | | | | observed. | | | | | | |
| 962 | 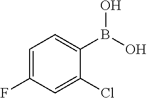 [SMILES: OB(O)c1ccc(F)cc1Cl] | 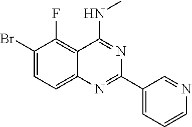 [SMILES: CNc1nc(-c2cccnc2)nc2ccc(Br)c(F)c12] | 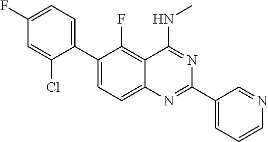 [SMILES: CNc1nc(-c2cccnc2)nc2ccc(-c3ccc(F)cc3Cl)c(F)c12] | 2HCl | 1H NMR (400 MHz, DMSO) δ 9.67 (d, J = 2.0 Hz, 1H), 9.16 (s, 1H), 8.94 (t, J = 4.4, 4.4 Hz, 1H), 8.47 (s, 1H), 7.96 (s, 1H), 7.91-7.76 (m, 2H), 7.71 (dd, | DMSO | >98 | Method R3 Temper- ature at 80° C. | | | |
| | | | | | J = 8.9, 2.6 Hz, | | | | | | |
| | | | | | 1H), 7.63 (dd, J = | | | | | | |
| | | | | | 8.6, 6.2 Hz, 1H), | | | | | | |
| | | | | | 7.43 (td, J = 8.5, | | | | | | |
| | | | | | 8.5, 2.7 Hz, 1H), | | | | | | |
| | | | | | 3.22 (d, J = 4.4 | | | | | | |
| | | | | | Hz, 3H). 1H of | | | | | | |
| | | | | | 2HC□ was not | | | | | | |
| | | | | | observed. | | | | | | |
| 963 | 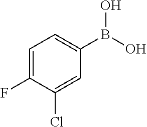 [SMILES: OB(O)c1ccc(F)c(Cl)c1] | 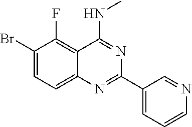 [SMILES: CNc1nc(-c2cccnc2)nc2ccc(Br)c(F)c12] | 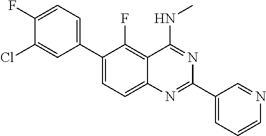 [SMILES: CNc1nc(-c2cccnc2)nc2ccc(-c3ccc(F)c(Cl)c3)c(F)c12] | 2HCl | 1H NMR (400 MHz, DMSO) δ 9.67 (d, J = 2.0 Hz, 1H), 9.21 (d, J = 8.1 Hz, 1H), 8.97 (dd, J = 5.3, 1.5 Hz, 1H), 8.58 (s, 1H), 8.09-7.97 (m, 1H), 7.94 (d, J = | DMSO | >98 | Method R3 Temper- ature at 80° C. | | | |
| | | | | | 8.4 Hz, 1H), 7.84 | | | | | | |
| | | | | | (d, J = 8.6 Hz, 1H), | | | | | | |
| | | | | | 7.75-7.67 (m, | | | | | | |
| | | | | | 1H), 7.67-7.58 (m, | | | | | | |
| | | | | | 2H), 3.24 (d, J = | | | | | | |
| | | | | | 4.4 Hz, 3H). 1H of | | | | | | |
| | | | | | 2HC□ was not | | | | | | |
| | | | | | observed. | | | | | | |
| 964 | 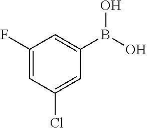 [SMILES: OB(O)c1cc(F)cc(Cl)c1] | 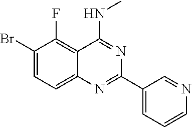 [SMILES: CNc1nc(-c2cccnc2)nc2ccc(Br)c(F)c12] | 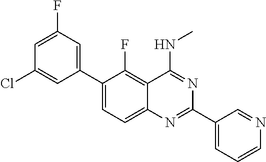 [SMILES: CNc1nc(-c2cccnc2)nc2ccc(-c3cc(F)cc(Cl)c3)c(F)c12] | 2HCl | 1H NMR (400 MHz, DMSO) δ 9.67 (d,J = 2.0 Hz, 1H), 9.15 (d, J = 8.0 Hz, 1H), 8.93 (dd, J = 5.2, 1.6 Hz, 1H), 8.50 (s, 1H), 8.06 (t, J = 8.5, 8.5 Hz, 1H), 7.94 (dd, J = 8.1, 5.3 Hz, 1H), 7.80 (d, | DMSO | >98 | Method R3 Temper- ature at 80° C. | | | |
| | | | | | J = 8.7 Hz, 1H), | | | | | | |
| | | | | | 7.71-7.63 (m, | | | | | | |
| | | | | | 1H), 7.58 (dd, J = | | | | | | |
| | | | | | 9.1, 1.7 Hz, 2H), | | | | | | |
| | | | | | 3.23 (d, J = 4.4 | | | | | | |
| | | | | | Hz, 3H). 1H of | | | | | | |
| | | | | | 2HC□ was not | | | | | | |
| | | | | | observed. | | | | | | |
| 965 | 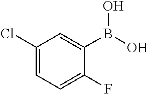 [SMILES: OB(O)c1cc(Cl)ccc1F] | 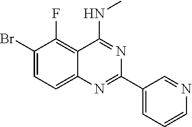 [SMILES: CNc1nc(-c2cccnc2)nc2ccc(Br)c(F)c12] | 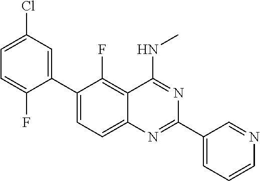 [SMILES: CNc1nc(-c2cccnc2)nc2ccc(-c3cc(Cl)ccc3F)c(F)c12] | 2HCl | 1H NMR (400 MHz, DMSO) δ 9.68 (d, J = 2.0 Hz, 1H), 9.21 (d, J = 8.1 Hz, 1H), 8.96 (dd, J = 5.3, 1.5 Hz, 1H), 8.54 (brd, J = 12.1 Hz, 1H), 8.06-7.94 (m, 2H), 7.84 (d, J = 8.6 Hz, 1H), 7.73 (dd, J = 6.3, 2.7 | DMSO | >98 | Method R3 Temper- ature at 80° C. | | | |
| | | | | | Hz, 1H), 7.64 (ddd, | | | | | | |
| | | | | | J = 8.8, 4.3, 2.7 | | | | | | |
| | | | | | Hz, 1H), 7.58-7.41 | | | | | | |
| | | | | | (m, 1H), 3.23 | | | | | | |
| | | | | | (d, J = 4.4 Hz, 3H). | | | | | | |
| | | | | | 1H of 2HC□ was | | | | | | |
| | | | | | not observed. | | | | | | |
| 966 | 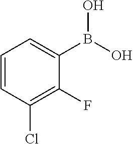 [SMILES: OB(O)c1cccc(Cl)c1F] | 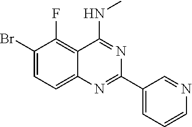 [SMILES: CNc1nc(-c2cccnc2)nc2ccc(Br)c(F)c12] | 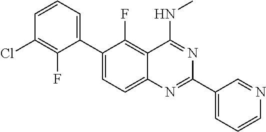 [SMILES: CNc1nc(-c2cccnc2)nc2ccc(-c3cccc(Cl)c3F)c(F)c12] | 2HCl | 1H NMR (400 MHz, DMSO) δ 9.68 (d, J = 2.0 Hz, 1H), 9.17 (d, J = 8.0 Hz, 1H), 8.94 (d, J = 5.3 Hz, 1H), 8.50 (brd, J = 9.0 Hz, 1H), 8.05- 7.92 (m, 2H), 7.84 | DMSO | >98 | Method R3 Temper- ature at 80° C. | | | |
| | | | | | (d, J = 8.6 Hz, | | | | | | |
| | | | | | 1H), 7.80-7.69 | | | | | | |
| | | | | | (m, 1H), 7.66- | | | | | | |
| | | | | | 7.53 (m, 1H), 7.43 | | | | | | |
| | | | | | (t, J = 7.9, 7.9 | | | | | | |
| | | | | | Hz, 1H), 3.23 (d, | | | | | | |
| | | | | | J = 4.4 Hz, 3H). | | | | | | |
| | | | | | 1H of 2HC□ was | | | | | | |
| | | | | | not observed. | | | | | | |
| 967 | 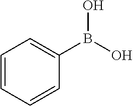 | 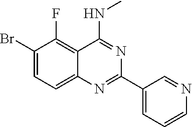 [SMILES: CNc1nc(-c2cccnc2)nc2ccc(Br)c(F)c12] | 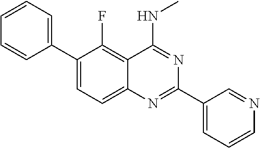 [SMILES: CNc1nc(-c2cccnc2)nc2ccc(-c3ccccc3)c(F)c12] | 2HCl | 1H NMR (400 MHz, DMSO) δ 9.66 (d,J = 2.0 Hz, 1H), 9.12 (s, 1H), 8.92 (d, J = 4.8 Hz, 1H), 8.53 (s, 1H), 8.02 (t, J = 8.5, 8.5 Hz, 1H), 7.93 (S, 1H), 7.82 (dd, J = 8.8, 2.5 | DMSO | >98 | Method R3 Temper- ature at 80° C. | | | |
| | | | | | Hz, 1H), 7.74-7.63 | | | | | | |
| | | | | | (m, 2H), 7.56 | | | | | | |
| | | | | | (t, J = 7.6, 7.6 Hz, | | | | | | |
| | | | | | 2H), 7.53-7.43 | | | | | | |
| | | | | | (m, 1H), 3.24 (d, | | | | | | |
| | | | | | J = 4.4 Hz, 3H). | | | | | | |
| | | | | | 1H of 2HC□ was | | | | | | |
| | | | | | not observed. | | | | | | |
| 968 | 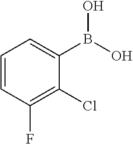 [SMILES: OB(O)c1cccc(F)c1Cl] | 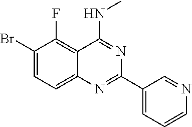 [SMILES: CNc1nc(-c2cccnc2)nc2ccc(Br)c(F)c12] | 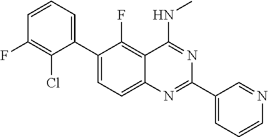 [SMILES: CNc1nc(-c2cccnc2)nc2ccc(-c3cccc(F)c3Cl)c(F)c12] | 2HCl | 1H NMR (400 MHz, DMSO) δ 9.76- 9.58 (m, 1H), 9.04 (d, J = 8.0 Hz, 1H), 8.87 (dd, J = 5.2, 1.6 Hz, 1H), 8.32 (brs, 1H), 7.93- 7.80 (m, 2H), 7.81-7.74 (m, | DMSO | >98 | Method R3 Temper- ature at 80° C. | | | |
| | | | | | 1H), 7.63-7.53 | | | | | | |
| | | | | | (m, 2H), 7.43 (s, | | | | | | |
| | | | | | 1H), 3.21 (d, J = | | | | | | |
| | | | | | 4.3 Hz, 3H). 1H of | | | | | | |
| | | | | | 2HC□ was not | | | | | | |
| | | | | | observed. | | | | | | |
| 969 | 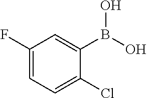 [SMILES: OB(O)c1cc(F)ccc1Cl] | 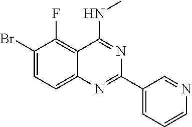 [SMILES: CNc1nc(-c2cccnc2)nc2ccc(Br)c(F)c12] | 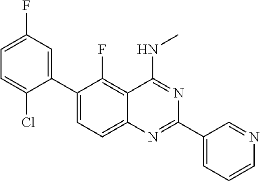 [SMILES: CNc1nc(-c2cccnc2)nc2ccc(-c3cc(F)ccc3Cl)c(F)c12] | 2HCl | 1H NMR (400 MHz, DMSO) δ 9.68 (d, J = 1.9 Hz, 1H), 9.19 (d, J = 8.0 Hz, 1H), 8.96 (dd, J = 5.3, 1.6 Hz, 1H), 8.49 (s, 1H), 7.99 (dd, J = 8.1, 5.4 Hz, 1H), 7.95- 7.80 (m, 2H), 7.74 (dd, J = 8.9, 5.2 Hz, 1H), 7.54 | DMSO | >98 | Method R3 Temper- ature at 80° C. | | | |
| | | | | | (dd, J = 9.0, 3.1 | | | | | | |
| | | | | | Hz, 1H), 7.51- | | | | | | |
| | | | | | 7.35 (m, 1H), 3.22 | | | | | | |
| | | | | | (d, J = 4.4 Hz, | | | | | | |
| | | | | | 3H). 1H of 2HC□ | | | | | | |
| | | | | | was not | | | | | | |
| | | | | | observed. | | | | | | |
| 970 | 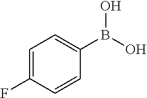 | 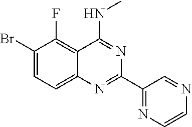 [SMILES: CNc1nc(-c2cnccn2)nc2ccc(Br)c(F)c12] | 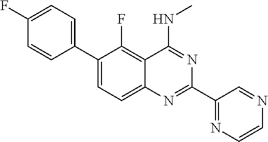 [SMILES: CNc1nc(-c2cnccn2)nc2ccc(-c3ccc(F)cc3)c(F)c12] | HCl | 1H NMR (400 MHz, DMSO) δ 9.80 (d, J = 1.5 Hz, 1H), 9.31 (s, 1H), 8.99 (dd, J = 16.5, 2.5 Hz, 2H), 8.15 (t, J = 8.3, 8.3 Hz, 1H), 8.03 (d, J = 8.5 Hz, 1H), | DMSO | >98 | Method R3 | | | |
| | | | | | 7.73 (dd, J = | | | | | | |
| | | | | | 8.6, 5.5 Hz, 2H), | | | | | | |
| | | | | | 7.53-7.36 (m, | | | | | | |
| | | | | | 2H), 3.34 (d, J = | | | | | | |
| | | | | | 4.9 Hz, 3H). 1H of | | | | | | |
| | | | | | HC□ was not | | | | | | |
| | | | | | observed. | | | | | | |
| 971 | 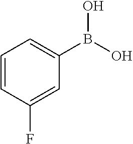 | 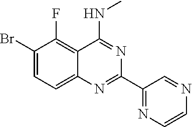 [SMILES: CNc1nc(-c2cnccn2)nc2ccc(Br)c(F)c12] | 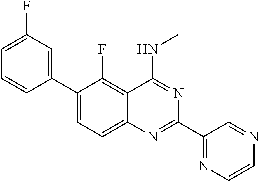 [SMILES: CNc1nc(-c2cnccn2)nc2ccc(-c3cccc(F)c3)c(F)c12] | HCl | 1H NMR (400 MHz, DMSO) δ 9.80 (s, 1H), 9.30 (s, 1H), 9.08-8.91 (m, 2H), 8.18 (s,1H), 8.03 (d, J = 8.8 Hz, 1H), 7.71- 7.59 (m, 1H), 7.59-7.48 (m, 2H), 7.44-7.26 (m, 1H), 3.35 (brd, 3H). 1H of HC□ was not observed. | DMSO | >98 | Method R3 | | | |
| 972 | 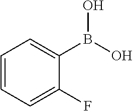 | 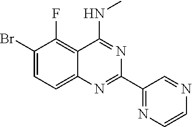 [SMILES: CNc1nc(-c2cnccn2)nc2ccc(Br)c(F)c12] | 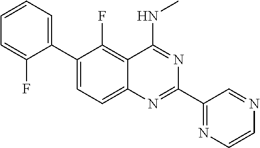 [SMILES: CNc1nc(-c2cnccn2)nc2ccc(-c3ccccc3F)c(F)c12] | HCl | 1H NMR (400 MHz, DMSO) δ 9.80 (d, J = 1.5 Hz, 1H), 9.30 (s, 1H), 9.11-8.90 (m, 2H), 8.23-7.99 (m, 2H), 7.60 (tdd, J = 7.2, 7.2, 3.6, 1.5 Hz, 2H), 7.51- 7.34 (m, 2H), | DMSO | >98 | Method R3 | | | |
| | | | | | 3.33 (d, J = 4.6 Hz, | | | | | | |
| | | | | | 3H) 1H of HC□ | | | | | | |
| | | | | | was not observed. | | | | | | |
| 973 | 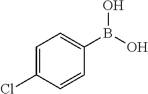 | 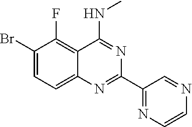 [SMILES: CNc1nc(-c2cnccn2)nc2ccc(Br)c(F)c12] | 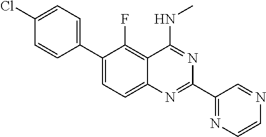 [SMILES: CNc1nc(-c2cnccn2)nc2ccc(-c3ccc(Cl)cc3)c(F)c12] | HCl | 1H NMR (400 MHz, DMSO) δ 9.94- 9.70 (m, 1H), 9.25 (s, 1H), 9.07- 8.91 (m, 2H), 8.15 (t, J = 8.4, 8.4 Hz, 1H), 8.02 (d, J = 9.4 Hz, 1H), 7.84-7.50 (m, 4H), | DMSO | >98 | Method R3 Temper- ature at 80° C. | | | |
| | | | | | 3.33 (d, J = | | | | | | |
| | | | | | 4.4 Hz, 3H). 1H of | | | | | | |
| | | | | | HC□ was not | | | | | | |
| | | | | | observed. | | | | | | |
| 974 | 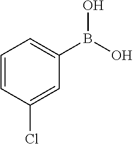 | 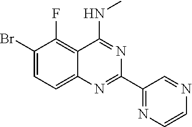 [SMILES: CNc1nc(-c2cnccn2)nc2ccc(Br)c(F)c12] | 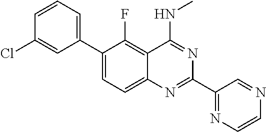 [SMILES: CNc1nc(-c2cnccn2)nc2ccc(-c3cccc(Cl)c3)c(F)c12] | HCl | 1H NMR (400 MHz, DMSO) δ 9.79 (s, 1H), 9.25 (s, 1H), 9.09-8.90 (m, 2H), 8.17 (t, J = 8.3, 8.3 Hz, 1H), 8.02 (d, J = 8.7 Hz, 1H), 7.78 (s, 1H), 7.73-7.49 | DMSO | >98 | Method R3 Temper- ature at 80° C. | | | |
| | | | | | (m, 3H), 3.33 | | | | | | |
| | | | | | (d, J = 4.4 Hz, 3H). | | | | | | |
| | | | | | 1H of HC□ | | | | | | |
| | | | | | was not observed. | | | | | | |
| 975 | 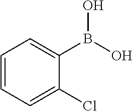 | 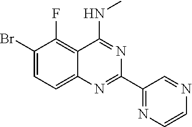 [SMILES: CNc1nc(-c2cnccn2)nc2ccc(Br)c(F)c12] | 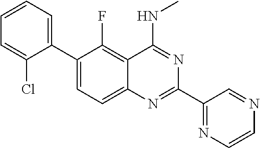 [SMILES: CNc1nc(-c2cnccn2)nc2ccc(-c3ccccc3Cl)c(F)c12] | HCl | 11H NMR (400 MHz, DMSO) δ 9.77 (s, 1H), 9.40-8.58 (m, 3H), 7.99 (s, 2H), 7.78-7.65 (m, 1H), 7.55 (q, J = 3.7, 3.1, 3.1 Hz, 3H), 3.29 (brd, 3H). 1H of HC□ was not observed. | DMSO | >98 | Method R3 Temper- ature at 80° C. | | | |
| 976 | 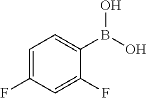 [SMILES: OB(O)c1ccc(F)cc1F] | 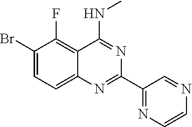 [SMILES: CNc1nc(-c2cnccn2)nc2ccc(Br)c(F)c12] | 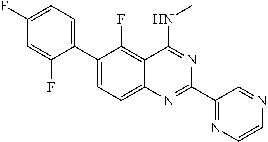 [SMILES: CNc1nc(-c2cnccn2)nc2ccc(-c3ccc(F)cc3F)c(F)c12] | HCl | 1H NMR (400 MHz, DMSO) δ 9.78 (s, 1H), 9.16 (s, 1H), 9.05-8.87 (m, 2H), 8.17-7.95 (m, 2H), 7.68 (td, J = 8.5, 8.5, 6.4 Hz, 1H), 7.53 (td, J = 9.9, 9.9, 2.5 | DMSO | >98 | Method R3 | | | |
| | | | | | Hz, 1H), 7.34 | | | | | | |
| | | | | | (td, J = 8.6, 8.5, 2.5 | | | | | | |
| | | | | | Hz, 1H), 3.31 | | | | | | |
| | | | | | (brd, 3H). 1H of | | | | | | |
| | | | | | HC□ was not | | | | | | |
| | | | | | observed. | | | | | | |
| 977 | 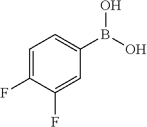 [SMILES: OB(O)c1ccc(F)c(F)c1] | 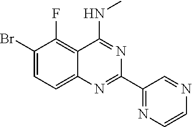 [SMILES: CNc1nc(-c2cnccn2)nc2ccc(Br)c(F)c12] | 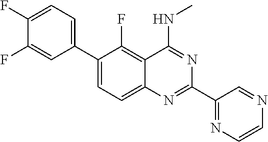 [SMILES: CNc1nc(-c2cnccn2)nc2ccc(-c3ccc(F)c(F)c3)c(F)c12] | HCl | 1H NMR (400 MHz, DMSO) δ 9.79 (s, 1H), 9.22 (s, 1H), 9.10-8.83 (m, 2H), 8.15 (t, J = 8.3, 8.3 Hz, 1H), 8.01 (d, J = 8.8 Hz, 1H), 7.92- 7.77 (m, 1H), 7.67 (dt, J = 10.5, | DMSO | >98 | Method R3 | | | |
| | | | | | 8.6, 8.6 Hz, 1H), | | | | | | |
| | | | | | 7.55 (t, J = 6.1, | | | | | | |
| | | | | | 6.1 Hz, 1H), 3.32 | | | | | | |
| | | | | | (brd, 3H). 1H of | | | | | | |
| | | | | | HC□ was not | | | | | | |
| | | | | | observed. | | | | | | |
| 978 | 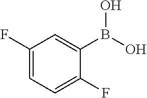 [SMILES: OB(O)c1cc(F)ccc1F] | 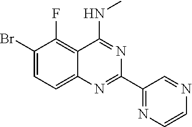 [SMILES: CNc1nc(-c2cnccn2)nc2ccc(Br)c(F)c12] | 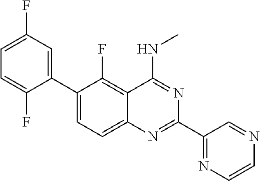 [SMILES: CNc1nc(-c2cnccn2)nc2ccc(-c3cc(F)ccc3F)c(F)c12] | HCl | 1H NMR (400 MHz, DMSO) δ 9.80 (d, J = 1.5 Hz, 1H), 9.32 (s, 1H), 9.13-8.85 (m, 2H), 8.38-7.86 (m, 2H), 7.70-7.26 (m, 3H), 3.34 (d, J = 4.5 H7, 3H). 1H of HC□ was not observed. | DMSO | >98 | Method R3 | | | |
| 979 | 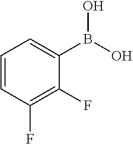 [SMILES: OB(O)c1cccc(F)c1F] | 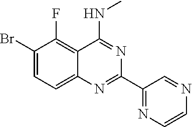 [SMILES: CNc1nc(-c2cnccn2)nc2ccc(Br)c(F)c12] | 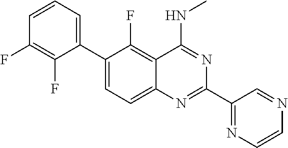 [SMILES: CNc1nc(-c2cnccn2)nc2ccc(-c3cccc(F)c3F)c(F)c12] | HCl | 1H NMR (400 MHz, DMSO) δ 9.77 (s, 1H), 9.17-8.72 (m, 3H), 8.17- 7.84 (m, 2H), 7.75- 7.47 (n % 1H), 7.43 (t, J = 4.4, 4.4 Hz, 2H), 3.30 (d, J = 4.6 Hz, 3H). 1H of HC□ was not observed. | DMSO | >98 | Method R3 | | | |
| 980 | 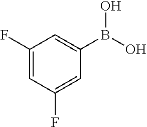 [SMILES: OB(O)c1cc(F)cc(F)c1] | 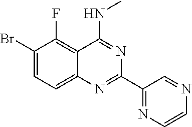 [SMILES: CNc1nc(-c2cnccn2)nc2ccc(Br)c(F)c12] | 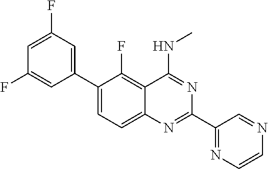 [SMILES: CNc1nc(-c2cnccn2)nc2ccc(-c3cc(F)cc(F)c3)c(F)c12] | HCl | 1H NMR (400 MHz, DMSO) δ 9.79 (d, J = 1.6 Hz, 1H), 9.28 (s, 1H), 9.14-8.79 (m, 2H), 8.19 (t, J = 8.4,8.4 Hz, 1H), 8.02 (d, J = 8.7 Hz, 1H), 7.61-7.12 (m, 3H), 3.34 (d, J = 4.6 Hz, 3H). | DMSO | >98 | Method R3 | | | |
| | | | | | 1H of HC□ was | | | | | | |
| | | | | | not observed. | | | | | | |
| 981 | 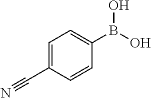 [SMILES: N#Cc1ccc(B(O)O)cc1] | 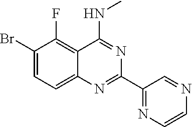 [SMILES: CNc1nc(-c2cnccn2)nc2ccc(Br)c(F)c12] | 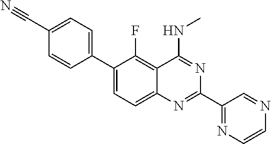 [SMILES: CNc1nc(-c2cnccn2)nc2ccc(-c3ccc(C#N)cc3)c(F)c12] | HCl | 1H NMR (400 MHz, DMSO) δ 9.80 (d, J = 1.5 Hz, 1H), 9.29 (s, 1H), 9.10-8.86 (m, 2 H), 8.19 (t, J = 8.3, 8.3 Hz, 1H), 8.13-8.02 (m, 3H), 7.89 (dd, J = | DMSO | >98 | Method R3 | | | |
| | | | | | 8.5, 1.5 Hz, 2H), | | | | | | |
| | | | | | 3.34 (d, J = 4.5 Hz, | | | | | | |
| | | | | | 3H). 1H of HC□ | | | | | | |
| | | | | | was not observed. | | | | | | |
| 982 | 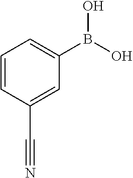 [SMILES: N#Cc1cccc(B(O)O)c1] | 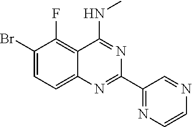 [SMILES: CNc1nc(-c2cnccn2)nc2ccc(Br)c(F)c12] | 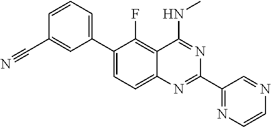 [SMILES: CNc1nc(-c2cnccn2)nc2ccc(-c3cccc(C#N)c3)c(F)c12] | HCl | 1H NMR (400 MHz, DMSO) δ 9.82 (d, J = 1.4 Hz, 1H), 9.51 (br, 1H), 9.13-8.89 (m, 2H), 8.25 (t, J = 8.3, 8.3 Hz, 1H), 8.19 (t, J = 1.5, 1.5 Hz, 1H), 8.09 (d, J = 8.7 Hz, 1H), 8.07-7.98 | DMSO | >98 | Method R3 | | | |
| | | | | | (m, 2H), 7.89- | | | | | | |
| | | | | | 7.75 (m, 1H), 3.38 | | | | | | |
| | | | | | (brd, 3H). 1H of | | | | | | |
| | | | | | HC□ was | | | | | | |
| | | | | | not observed. | | | | | | |
| 983 | 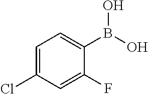 [SMILES: OB(O)c1ccc(Cl)cc1F] | 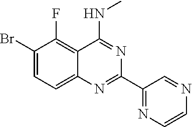 [SMILES: CNc1nc(-c2cnccn2)nc2ccc(Br)c(F)c12] | 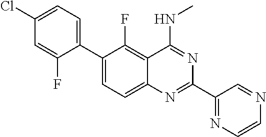 [SMILES: CNc1nc(-c2cnccn2)nc2ccc(-c3ccc(Cl)cc3F)c(F)c12] | HCl | 1H NMR (400 MHz, DMSO) δ 9.78 (d, J = 1.5 Hz, 1H), 9.18 (s, 1H), 9.05-8.86 (m, 2H), 8.16-7.90 (m, 2H), 7.72 (dd, J = 10.0, 2.1 Hz, 1H), 7.65 (t, J = | DMSO | >98 | Method R3 Temper- ature at 80° C. | | | |
| | | | | | 8.2, 8.2 Hz, 1H), | | | | | | |
| | | | | | 7.53 (dd, J = 8.3, | | | | | | |
| | | | | | 2.1 Hz, 1H), 3.32 | | | | | | |
| | | | | | (d, J = 4.5 Hz, 3H). | | | | | | |
| | | | | | 1H of HC□ was | | | | | | |
| | | | | | not observed. | | | | | | |
| 984 | 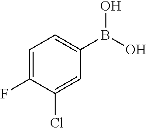 [SMILES: OB(O)c1ccc(F)c(Cl)c1] | 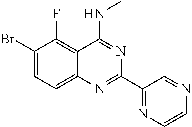 [SMILES: CNc1nc(-c2cnccn2)nc2ccc(Br)c(F)c12] | 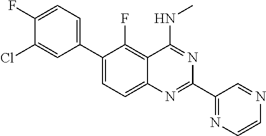 [SMILES: CNc1nc(-c2cnccn2)nc2ccc(-c3ccc(F)c(Cl)c3)c(F)c12] | HCl | 1H NMR (400 MHz, DMSO) δ 9.79 (d, J = 1.5 Hz, 1H), 9.32 (s, 1H), 8.99 (dd, J = 16.1, 2.4 Hz, 2H), 8.18 (t, J = 8.4, 8.4 Hz, 1H), 8.03 (d, J = 8.7 Hz, 1H), | DMSO | >98 | Method R3 Temper- ature at 80° C. | | | |
| | | | | | 7.95 (dt, J = | | | | | | |
| | | | | | 7.0, 1.8, 1.8 Hz, | | | | | | |
| | | | | | 1H), 7.82-7.51 | | | | | | |
| | | | | | (m, 2H), 3.34 (d, | | | | | | |
| | | | | | J = 4.7 Hz, 3H). | | | | | | |
| | | | | | 1H of HC□ was | | | | | | |
| | | | | | not observed. | | | | | | |
| 985 | 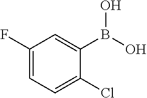 [SMILES: OB(O)c1cc(F)ccc1Cl] | 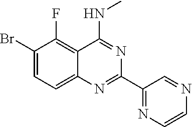 [SMILES: CNc1nc(-c2cnccn2)nc2ccc(Br)c(F)c12] | 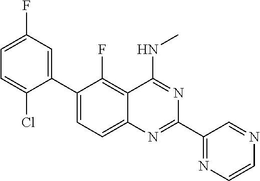 [SMILES: CNc1nc(-c2cnccn2)nc2ccc(-c3cc(F)ccc3Cl)c(F)c12] | HCl | 1H NMR (400 MHz, DMSO) δ 9.71 (s, 1H), 8.89 (s, 2H), 7.89 (s, 2H), 7.74 (dd, J = 9.0, 5.2 Hz, 1H), 7.55 (dd, J = 9.0, 3.1 Hz, 1H), 7.44 (td, J = 8.7, 8.6, 3.2 Hz, 1H), 3.23 (s, 3H). 1H of NH and HCl were not observed. | DMSO | >98 | Method R3 Temper- ature at 80° C. | | | |
| 986 | 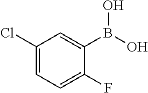 [SMILES: OB(O)c1cc(Cl)ccc1F] | 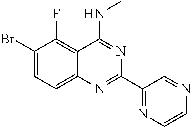 [SMILES: CNc1nc(-c2cnccn2)nc2ccc(Br)c(F)c12] | 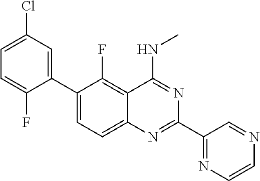 [SMILES: CNc1nc(-c2cnccn2)nc2ccc(-c3cc(Cl)ccc3F)c(F)c12] | HCl | 1H NMR (400 MHz, DMSO) δ 9.79 (d, J = 1.5 Hz, 1H), 9.24 (s, 1H), 9.09-8.90 (m, 2H), 8.11 (t, J = 8.0, 8.0 Hz, 1H), 8.07-7.98 (m, 1H), 7.75 (dd, J = 6.3, 2.7 Hz, 1H), 7.67 (ddd, J = 8.8, 4.4, 2.7 Hz, 1H), | DMSO | >98 | Method R3 Temper- ature at 80° C. | | | |
| | | | | | 7.60-7.22 (m, | | | | | | |
| | | | | | 1H), 3.32 (d, J = | | | | | | |
| | | | | | 4.5 Hz, 3H). 1H of | | | | | | |
| | | | | | HCl was not | | | | | | |
| | | | | | observed. | | | | | | |
| 987 | 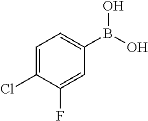 [SMILES: OB(O)c1ccc(Cl)c(F)c1] | 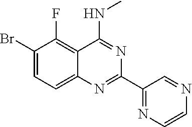 [SMILES: CNc1nc(-c2cnccn2)nc2ccc(Br)c(F)c12] | 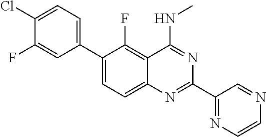 [SMILES: CNc1nc(-c2cnccn2)nc2ccc(-c3ccc(Cl)c(F)c3)c(F)c12] | HCl | 1H NMR (400 MHz, DMSO) δ 9.79 (d, J= 1.6 Hz, 1H), 9.22 (s, 1H), 9.06-8.90 (m, 2H), 8.17 (t, J = 8.4, 8.4 Hz, 1H), 8.02 (d, J = 8.8 Hz, 1H), 7.90- | DMSO | >98 | Method R3 Temper- ature at 80° C. | | | |
| | | | | | 7.68 (m, 2H), 7.56 | | | | | | |
| | | | | | (d, J = 8.4 Hz, 1H), | | | | | | |
| | | | | | 3.33 (d, J = 4.5 | | | | | | |
| | | | | | Hz, 3H). 1H of | | | | | | |
| | | | | | HCl was not | | | | | | |
| | | | | | observed. | | | | | | |
| 988 | 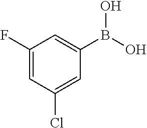 [SMILES: OB(O)c1cc(F)cc(Cl)c1] | 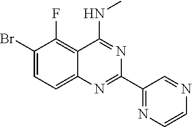 [SMILES: CNc1nc(-c2cnccn2)nc2ccc(Br)c(F)c12] | 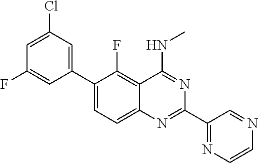 [SMILES: CNc1nc(-c2cnccn2)nc2ccc(-c3cc(F)cc(Cl)c3)c(F)c12] | HCl | 1H NMR (400 MHz, DMSO) δ 9.76 (s, 1H), 8.94 (s, 3H), 8.14 (d, J = 8.5 Hz, 1H), 7.95 (d, J = 8.7 Hz, 1H), 7.69-7.64 (m, 1H), 7.61 (ddd, J = 9.6, 5.6, 3.7 Hz, 2H), 3.29 | DMSO | >98 | Method R3 Temper- ature at 80° C. | | | |
| | | | | | (d, J = 4.4 Hz, | | | | | | |
| | | | | | 3H). 1H of HCl was | | | | | | |
| | | | | | not observed. | | | | | | |
| 989 | 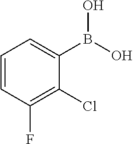 [SMILES: OB(O)c1cccc(F)c1Cl] | 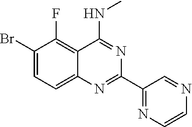 [SMILES: CNc1nc(-c2cnccn2)nc2ccc(Br)c(F)c12] | 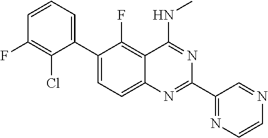 [SMILES: CNc1nc(-c2cnccn2)nc2ccc(-c3cccc(F)c3Cl)c(F)c12] | HCl | 1H NMR (400 MHz, DMSO) δ 9.81 (d, J = 1.5 Hz, 1H), 9.33 (s, 1H), 9.19-8.88 (m, 2H), 8.17-7.87 (m, 2H), 7.78-7.50 (m, 2H), 7.50- 7.30 (m, 1H), 3.33 | DMSO | >98 | Method R3 Temper- ature at 80° C. | | | |
| | | | | | (d, J = 4.5 Hz, | | | | | | |
| | | | | | 3H). | | | | | | |
| | | | | | 1H of HCl was | | | | | | |
| | | | | | not observed. | | | | | | |
| 990 | 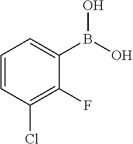 [SMILES: OB(O)c1cccc(Cl)c1F] | 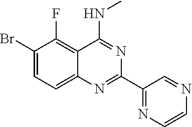 [SMILES: CNc1nc(-c2cnccn2)nc2ccc(Br)c(F)c12] | 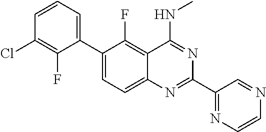 [SMILES: CNc1nc(-c2cnccn2)nc2ccc(-c3cccc(Cl)c3F)c(F)c12] | HCl | 1H NMR (400 MHz, DMSO) δ 9.80 (d, J = 1.5 Hz, 1H), 9.32 (s, 1H), 9.10-8.83 (m, 2H), 8.21-7.99 (m, 2H), 7.79 (ddd, J = 8.1, 7.2, 1.7 Hz, 1H), 7.59 (ddd, | DMSO | >98 | Method R3 Temper- ature at 80° C. | | | |
| | | | | | J = 8.1, 6.6, 1.7 | | | | | | |
| | | | | | Hz, 1H), 7.50-7.32 | | | | | | |
| | | | | | (m, 1H), 3.33 | | | | | | |
| | | | | | (d, J = 4.5 Hz, 3H). | | | | | | |
| | | | | | 1H Of HCl was | | | | | | |
| | | | | | not observed. | | | | | | |
| 991 | 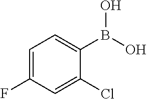 [SMILES: OB(O)c1ccc(F)cc1Cl] | 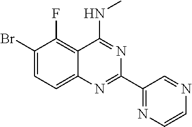 [SMILES: CNc1nc(-c2cnccn2)nc2ccc(Br)c(F)c12] | 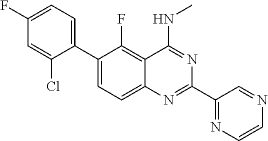 [SMILES: CNc1nc(-c2cnccn2)nc2ccc(-c3ccc(F)cc3Cl)c(F)c12] | HCl | 1H NMR (400 MHz, DMSO) δ 9.80 (d, J = 1.5 Hz, 1H), 9.31 (s, 1H), 9.15-8.91 (m, 2H), 8.14-7.89 (m, 2H), 7.74 (dd, J = 8.8, 2.6 Hz, 1H), 7.64 (dd, J = | DMSO | >98 | Method R3 Temper- ature at 80° C. | | | |
| | | | | | 8.6, 6.1 Hz, 1H), | | | | | | |
| | | | | | 7.46 (td,J = 8.5, | | | | | | |
| | | | | | 8.5, 2.7 Hz, 1H), | | | | | | |
| | | | | | 3.33 (d, J = 4.5 Hz, | | | | | | |
| | | | | | 3H). 1H of HCl | | | | | | |
| | | | | | was not observed. | | | | | | |
| 992 | 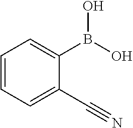 [SMILES: N#Cc1ccccc1B(O)O] | 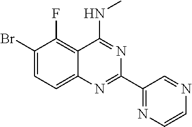 [SMILES: CNc1nc(-c2cnccn2)nc2ccc(Br)c(F)c12] | 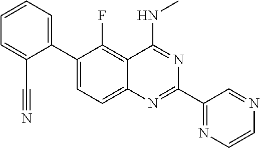 [SMILES: CNc1nc(-c2cnccn2)nc2ccc(-c3ccccc3C#N)c(F)c12] | HCl | 1H NMR (400 MHz, DMSO) δ 9.71 (d, J = 1.6 Hz, 1H), 8.98-8.77 (m, 2H), 8.08 (dd, J = 7.9, 1.3 Hz, 1H), 7.99 (d, J = 8.0 Hz, 1H), 7.91 (td, J = 7.7, 7.7, 1.3 Hz, 2H), 7.81-7.65 | DMSO | >98 | Method R3 Temper- ature at 80° C. | | | |
| | | | | | (m, 2H), 3.24 (d, | | | | | | |
| | | | | | J = 4.4 Hz, 3H). | | | | | | |
| | | | | | 1H of NH was | | | | | | |
| | | | | | not observed | | | | | | |
| 993 | 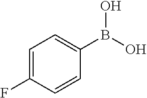 | 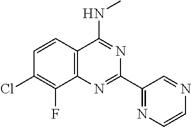 [SMILES: CNc1nc(-c2cnccn2)nc2c(F)c(Cl)ccc12] | 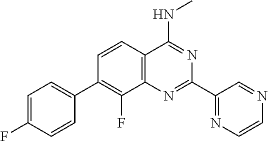 [SMILES: CNc1nc(-c2cnccn2)nc2c(F)c(-c3ccc(F)cc3)ccc12] | 2HCl | 1H NMR (400 MHz, DMSO) δ 9.67 (d, J = 1.4 Hz, 1H), 9.05 (s, 1H), 8.90-8.69 (m, 2H), 8.21 (d, J = 8.6 Hz, 1H), 7.88- 7.65 (m, 3H), 7.51-7.24 (m, | DMSO | >98 | Method R5 | | | |
| | | | | | 2H), 3.21 (d, J = | | | | | | |
| | | | | | 4.4 Hz, 3H). 1H of | | | | | | |
| | | | | | 2HCl was not | | | | | | |
| | | | | | observed. | | | | | | |
| 994 | 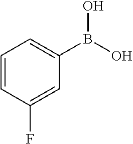 | 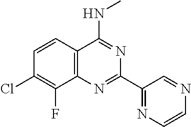 [SMILES: CNc1nc(-c2cnccn2)nc2c(F)c(Cl)ccc12] | 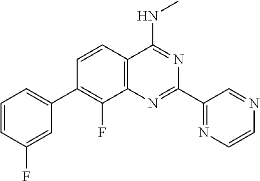 [SMILES: CNc1nc(-c2cnccn2)nc2c(F)c(-c3cccc(F)c3)ccc12] | HCl | 1H NMR (400 MHz, DMSO) δ 9.66 (d, J = 1.5 Hz, 1H), 8.95 (d, J = 5.5 Hz, 1H), 8.86 (dd, J = 2.4, 1.5 Hz, 1H), 8.80 (d, J = 2.4 Hz, 1H), 8.21 (d, J = 8.6 Hz, 1H), 7.77 (dd, J = 8.6, 7.0 Hz, 1H), 7.69-7.52 (m, | DMSO | >98 | Method R5 | | | |
| | | | | | 3H), 7.35 (dddd, | | | | | | |
| | | | | | J = 9.1, 7.8, 2.7, | | | | | | |
| | | | | | 1.4 Hz, 1H), 3.20 | | | | | | |
| | | | | | (d, J = 4.4 Hz, | | | | | | |
| | | | | | 3H). 1H of HCl was | | | | | | |
| | | | | | not observed. | | | | | | |
| 995 | 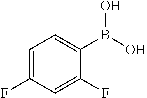 [SMILES: OB(O)c1ccc(F)cc1F] | 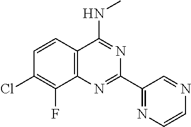 [SMILES: CNc1nc(-c2cnccn2)nc2c(F)c(Cl)ccc12] | 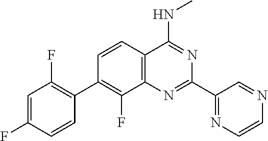 [SMILES: CNc1nc(-c2cnccn2)nc2c(F)c(-c3ccc(F)cc3F)ccc12] | HCl | 1H NMR (400 MHz, DMSO) δ 9.66 (d,J = 1.5 Hz, 1H), 8.88 (d, J = 5.0 Hz, 1H), 8.85 (dd, J = 2.4, 1.5 Hz, 1H), 8.79 (d, J = 2.4 Hz, 1H), 8.20 (d, J = 8.6 Hz, 1H), 7.76-7.58 (m, | DMSO | >98 | Method R5 | | | |
| | | | | | 2H), 7.50 (ddd, | | | | | | |
| | | | | | J = 10.4, 9.3, 2.6 | | | | | | |
| | | | | | Hz, 1H), 7.38-7.25 | | | | | | |
| | | | | | (m, 1H), 3.19 | | | | | | |
| | | | | | (d, J = 4.4 Hz, 3H). | | | | | | |
| | | | | | 1H of HCl was | | | | | | |
| | | | | | not observed. | | | | | | |
| 996 | 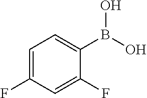 [SMILES: OB(O)c1ccc(F)cc1F] | 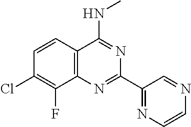 [SMILES: CNc1nc(-c2cnccn2)nc2c(F)c(Cl)ccc12] | 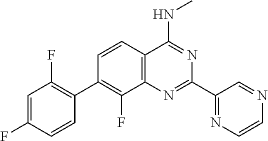 [SMILES: CNc1nc(-c2cnccn2)nc2c(F)c(-c3ccc(F)cc3F)ccc12] | MSA | 1H NMR (400 MHz, CDCl3) δ 11.98 (s, 1H), 9.88 (d, J = 1.4 Hz, 1H), 9.04 (d, J = 2.4 Hz, 1H), 8.99 (dd, J = 8.7, 1.2 Hz, 1H), 8.83 (dd, J = 2.4, 1.5 Hz, 1H), 7.82 (dd, J = 8.6, | DMSO | >98 | Method R5 | | | |
| | | | | | 7.1 Hz, 1H), 7.47 | | | | | | |
| | | | | | (td, J = 8.4, 8.4, | | | | | | |
| | | | | | 6.2 Hz, 1H), 7.13- | | | | | | |
| | | | | | 7.07 (m, 1H), | | | | | | |
| | | | | | 7.03 (ddd, J = | | | | | | |
| | | | | | 10.0, 8.7, 2.5 Hz, | | | | | | |
| | | | | | 1H), 3.63 (d, J = | | | | | | |
| | | | | | 4.7 Hz, 3H), 2.92 | | | | | | |
| | | | | | (s, 3H). 1H of | | | | | | |
| | | | | | MSA was not | | | | | | |
| | | | | | observed. | | | | | | |
| 997 | 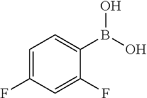 [SMILES: OB(O)c1ccc(F)cc1F] | 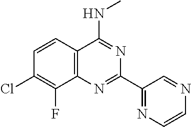 [SMILES: CNc1nc(-c2cnccn2)nc2c(F)c(Cl)ccc12] | 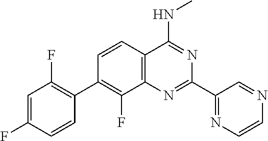 [SMILES: CNc1nc(-c2cnccn2)nc2c(F)c(-c3ccc(F)cc3F)ccc12] | TSA | 1H NMR (400 MHz, DMSO) δ 9.66 (d, J = 1.4 Hz, 1H), 8.90 (s, 1H), 8.85 (dd, J = 2.4, 1.5 Hz, 1H), 8.80 (d, J = 2.5 Hz, 1H), 8.19 (d, J = 8.6 Hz, 1H), 7.74-7.59 (m, 2H), 7.55- | DMSO | >98 | Method R5 | | | |
| | | | | | 7.43 (m, 3H), 7.35- | | | | | | |
| | | | | | 7.25 (m, 1H), | | | | | | |
| | | | | | 7.11 (d, J = 7.8 Hz, | | | | | | |
| | | | | | 2H), 3.20 (d, J = | | | | | | |
| | | | | | 4.4 Hz, 3H), 2.29 | | | | | | |
| | | | | | (s, 3H). 1H of | | | | | | |
| | | | | | TSA was not | | | | | | |
| | | | | | observed. | | | | | | |
| 998 | 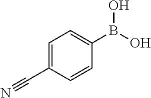 [SMILES: N#Cc1ccc(B(O)O)cc1] | 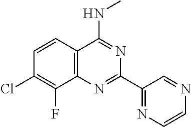 [SMILES: CNc1nc(-c2cnccn2)nc2c(F)c(Cl)ccc12] | 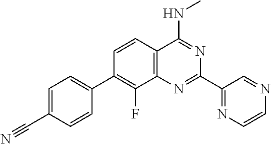 [SMILES: CNc1nc(-c2cnccn2)nc2c(F)c(-c3ccc(C#N)cc3)ccc12] | HCl | 1H NMR (400 MHz, DMSO) δ 9.66 (d, J = 1.5 Hz, 1H), 8.95 (d, J = 5.3 Hz, 1H), 8.86 (dd, J = 2.4, 1.5 Hz, 1H), 8.80 (d, J = 2.4 Hz, 1H), 8.23 (d, J = 8.7 Hz, 1H), 8.09-8.00 (m, | DMSO | >98 | Method R5 | | | |
| | | | | | 2H), 8.00-7.86 (m, | | | | | | |
| | | | | | 2H), 7.78 (dd, | | | | | | |
| | | | | | J = 8.6, 7.1 Hz, 1H), | | | | | | |
| | | | | | 3.20 (d, J = 4.4 | | | | | | |
| | | | | | Hz, 3H). | | | | | | |
| | | | | | 1H of HCl was | | | | | | |
| | | | | | not observed. | | | | | | |
| 999 | 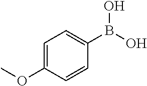 | 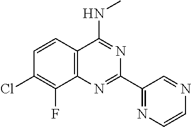 [SMILES: CNc1nc(-c2cnccn2)nc2c(F)c(Cl)ccc12] | 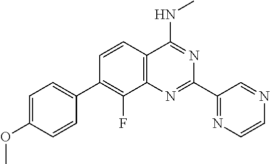 [SMILES: CNc1nc(-c2cnccn2)nc2c(F)c(-c3ccc(OC)cc3)ccc12] | HCl | 1H NMR (400 MHz, DMSO) δ 9.66 (d, J = 1.5 Hz, 1H), 8.98 (s, 1H), 8.86 (dd, J = 2.5, 1.5 Hz, 1H), 8.81 (d, J = 2.4 Hz, 1H), 8.17 (d, J = 8.7 Hz, 1H), 7.78- 7.62 (m, 3H), 7.24- 6.94 (m, 2H), 3.85 | DMSO | >98 | Method R5 | | | |
| | | | | | (s, 3H), 3.20 (d, | | | | | | |
| | | | | | J = 4.4 Hz, 3H). | | | | | | |
| | | | | | 1H of HCl was | | | | | | |
| | | | | | not observed. | | | | | | |
| 1000 | 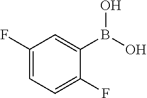 [SMILES: OB(O)c1cc(F)ccc1F] | 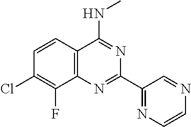 [SMILES: CNc1nc(-c2cnccn2)nc2c(F)c(Cl)ccc12] | 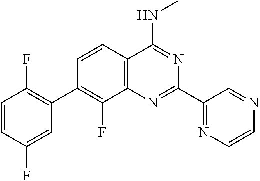 [SMILES: CNc1nc(-c2cnccn2)nc2c(F)c(-c3cc(F)ccc3F)ccc12] | HCl | 1H NMR (400 MHz, DMSO) δ 9.56 (d, J = 1.5 Hz, 1H), 8.88 (d, J = 5.9 Hz, 1H), 8.85 (t, J = 1.9, 1.9 Hz, 1H), 8.79 (d, J = 2.5 Hz, 1H), 8.21 (d, J = 8.6 Hz, 1H), 7.66 (dd, J = 8.5, 6.6 Hz, 1H), 7.60-7.39 (m, | DMSO | >98 | Method R5 | | | |
| | | | | | 3H), 3.19 (d, J = | | | | | | |
| | | | | | 4.4 Hz, 3H). 1H of | | | | | | |
| | | | | | HCl was not | | | | | | |
| | | | | | observed. | | | | | | |
| 1001 | 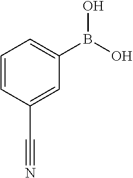 [SMILES: N#Cc1cccc(B(O)O)c1] | 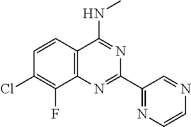 [SMILES: CNc1nc(-c2cnccn2)nc2c(F)c(Cl)ccc12] | 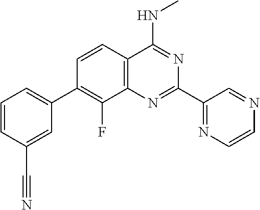 [SMILES: CNc1nc(-c2cnccn2)nc2c(F)c(-c3cccc(C#N)c3)ccc12] | HCl | 1H NMR (400 MHz, DMSO) δ 9.66 (d, J = 1.5 Hz, 1H), 8.88 (d, J = 4.6 Hz, 1H), 8.85 (dd, J = 2.4, 1.5 Hz, 1H), 8.79 (d, J = 2.4 Hz, 1H), 8.22 (dd, J = 4.9, 3.1 Hz, 2H), 8.08 (dt, J = 8.0,1.5,1.5 Hz, 1H), 7.98 (dt, J = 7.8, 1.4, 1.4 Hz, | DMSO | >98 | Method R5 | | | |
| | | | | | 1H), 7.79 (td, J = | | | | | | |
| | | | | | 7.9, 7.5, 4.1 Hz, | | | | | | |
| | | | | | 2H), 3.19 (d, J = | | | | | | |
| | | | | | 4.4 Hz, 3H). 1H | | | | | | |
| | | | | | of HCl was not | | | | | | |
| | | | | | observed. | | | | | | |
| 1002 | 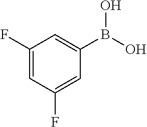 [SMILES: OB(O)c1cc(F)cc(F)c1] | 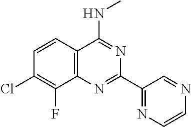 [SMILES: CNc1nc(-c2cnccn2)nc2c(F)c(Cl)ccc12] | 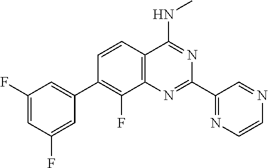 [SMILES: CNc1nc(-c2cnccn2)nc2c(F)c(-c3cc(F)cc(F)c3)ccc12] | HCl | 1H NMR (400 MHz, DMSO) δ 9.66 (d, J = 1.5 Hz, 1H), 8.90 (d, J = 5.3 Hz, 1H), 8.85 (dd, J = 2.4, 1.5 Hz, 1H), 8.79 (d, J = 2.5 Hz, 1H), 8.20 (d,J = 8.6 Hz, 1H), 7.78 (dd, J = 8.6, 7.0 Hz, 1H), | DMSO | >98 | Method R5 | | | |
| | | | | | 7.58-7.47 (m, | | | | | | |
| | | | | | 2H), 7.42 (tt, J = | | | | | | |
| | | | | | 9.4, 9.4, 2.4, 2.4 | | | | | | |
| | | | | | Hz, 1H), 3.19 (d, | | | | | | |
| | | | | | J = 4.4 Hz, 3H). | | | | | | |
| | | | | | 1H of HCl was | | | | | | |
| | | | | | not observed. | | | | | | |
| 1003 | 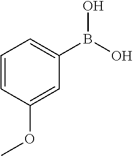 | 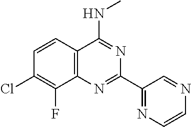 [SMILES: CNc1nc(-c2cnccn2)nc2c(F)c(Cl)ccc12] | 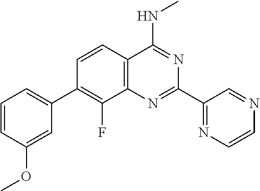 [SMILES: CNc1nc(-c2cnccn2)nc2c(F)c(-c3cccc(OC)c3)ccc12] | HCl | 1H NMR (400 MHz, DMSO) δ 9.67 (d, J = 1.5 Hz, 1H), 8.96 (s, 1H), 8.86 (dd, J = 2.4, 1.5 Hz, 1H), 8.81 (d, J = 2.5 Hz, 1H), 8.19 (d, J = 8.6 Hz, 1H), 7.75 (dd, J = 8.6, 7.1 Hz, 1H), 7.48 (t, J = 8.0, 8.0 Hz, 1H), 7.37-7.13 (m, | DMSO | >98 | Method R5 | | | |
| | | | | | 2H), 7.07 (ddd, J = | | | | | | |
| | | | | | 8.3, 2.7, 1.0 Hz, | | | | | | |
| | | | | | 1H), 3.85 (s, 3H), | | | | | | |
| | | | | | 3.20 (d, J = 4.4 Hz, | | | | | | |
| | | | | | 3H). 1H of HCl | | | | | | |
| | | | | | was not observed. | | | | | | |
| 1004 | 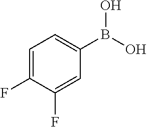 [SMILES: OB(O)c1ccc(F)c(F)c1] | 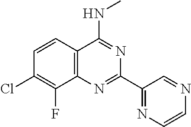 [SMILES: CNc1nc(-c2cnccn2)nc2c(F)c(Cl)ccc12] | 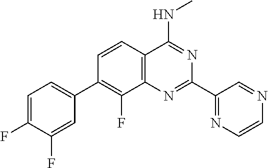 [SMILES: CNc1nc(-c2cnccn2)nc2c(F)c(-c3ccc(F)c(F)c3)ccc12] | HCl | 1H NMR (400 MHz, DMSO) δ 9.65 (d, J = 1.5 Hz, 1H), 8.92-8.81 (m, 2H), 8.79 (d, J = 2.5 Hz, 1H), 8.18 (d, J =8.7 Hz, 1H), 7.86 (t, J = 9.9, 9.9 Hz, 1H), 7.75 (dd, J-8.7, 7.1 Hz, 1H), 7.71-7.54 | DMSO | >98 | Method R5 | | | |
| | | | | | (m, 2H), 3.19 | | | | | | |
| | | | | | (d, J = 4.4 Hz, 3H). | | | | | | |
| | | | | | 1H of HCl was | | | | | | |
| | | | | | not observed. | | | | | | |
| 1005 | 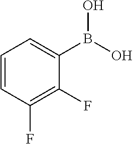 [SMILES: OB(O)c1cccc(F)c1F] | 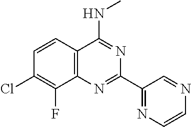 [SMILES: CNc1nc(-c2cnccn2)nc2c(F)c(Cl)ccc12] | 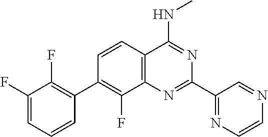 [SMILES: CNc1nc(-c2cnccn2)nc2c(F)c(-c3cccc(F)c3F)ccc12] | HCl | 1H NMR (400 MHz, DMSO) δ 9.66 (d, J = 1.5 Hz, 1H), 8.92-8.83 (m, 2H), 8.79 (d, J = 2.5 Hz, 1H), 8.21 (d, J-8.7 Hz, 1H), 7.75-7.55 (m, 2H), 7.52-7.35 (m, | DMSO | >98 | Method R5 | | | |
| | | | | | 2H), 3.19 (d, | | | | | | |
| | | | | | J = 4.4 Hz, 3H). 1H | | | | | | |
| | | | | | of HCl was not | | | | | | |
| | | | | | observed. | | | | | | |
| 1006 | 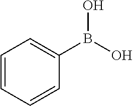 | 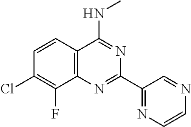 [SMILES: CNc1nc(-c2cnccn2)nc2c(F)c(Cl)ccc12] | 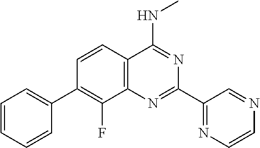 [SMILES: CNc1nc(-c2cnccn2)nc2c(F)c(-c3ccccc3)ccc12] | HCl | 1H NMR (400 MHz, DMSO) δ 9.66 (d, J = 1.5 Hz, 1H), 8.94 (s, 1H), 8.86 (dd, J = 2.5, 1.5 Hz, 1H), 8.80 (d, J = 2.5 Hz, 1H), 8.20 (d, J = 8.6 Hz, 1H), 7.78-7.68 (m, 3H), 7.64- | DMSO | >98 | Method R5 | | | |
| | | | | | 7.54 (m, 2H), 7.54- | | | | | | |
| | | | | | 7.43 (m, 1H), | | | | | | |
| | | | | | 3.20 (d, J = | | | | | | |
| | | | | | 4.4 Hz, 3H). | | | | | | |
| | | | | | 1H of HCl was | | | | | | |
| | | | | | not observed. | | | | | | |
| 1007 | 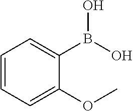 | 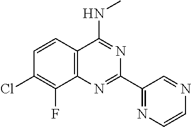 [SMILES: CNc1nc(-c2cnccn2)nc2c(F)c(Cl)ccc12] | 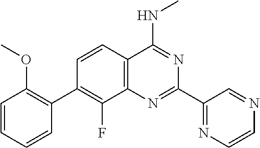 [SMILES: CNc1nc(-c2cnccn2)nc2c(F)c(-c3ccccc3OC)ccc12] | HCl | 1H NMR (400 MH7, DMSO) δ 9.66 (d, J = 1.5 Hz, 1H), 8.90 (s, 1H), 8.85 (dd, 3 = 2.4, 1.5 Hz, 1H), 8.80 (d, J = 2.5 Hz, 1H), 8.13 (d, J = 8.6 Hz, 1H), 7.60-7.44 (m, 2H), 7.37 | DMSO | >98 | Method R5 | | | |
| | | | | | (dd, J = 7.5, 1.7 Hz, | | | | | | |
| | | | | | 1H), 7.21 (dd, | | | | | | |
| | | | | | J = 8.5, 1.1 Hz, | | | | | | |
| | | | | | 1H), 7.11 (td, J = | | | | | | |
| | | | | | 7.4, 7.4, 1.0 Hz, | | | | | | |
| | | | | | 1H), 3.79 (s, 3H), | | | | | | |
| | | | | | 3.20 (d, J = 4.4 Hz, | | | | | | |
| | | | | | 3H). 1H of HCl | | | | | | |
| | | | | | was not observed. | | | | | | |
| 1008 | 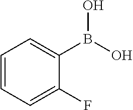 | 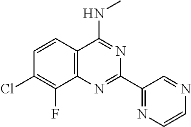 [SMILES: CNc1nc(-c2cnccn2)nc2c(F)c(Cl)ccc12] | 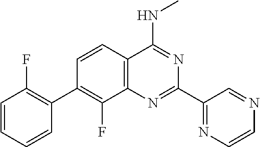 [SMILES: CNc1nc(-c2cnccn2)nc2c(F)c(-c3ccccc3F)ccc12] | HCl | 1H NMR (400 MHz, DMSO) δ 9.66 (d, J = 1.4 Hz, 1H), 8.92 (s, 1H), 8.85 (t, J = 2.0, 2.0 Hz, 1H), 8.80 (d, J = 2.5 Hz, 1H), 8.21 (d, J = 8.6 Hz, 1H), 7.70-7.53 (m, 3H), 7.50- | DMSO | >98 | Method R5 | | | |
| | | | | | 7.33 (m, 2H), 3.20 | | | | | | |
| | | | | | (d, J = 4.4 Hz, | | | | | | |
| | | | | | 3H). 1H of HCl was | | | | | | |
| | | | | | not observed. | | | | | | |
| 1009 | 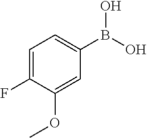 | 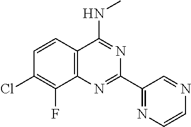 [SMILES: CNc1nc(-c2cnccn2)nc2c(F)c(Cl)ccc12] | 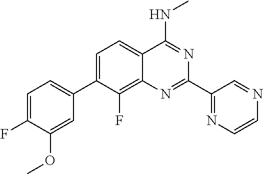 [SMILES: CNc1nc(-c2cnccn2)nc2c(F)c(-c3ccc(F)c(OC)c3)ccc12] | HCl | 1H NMR (400 MHz, DMSO) δ 9.66 (d, J = 1.5 Hz, 1H), 8.90 (s, 1H), 8.85 (dd, J = 2.5, 1.5 Hz, 1H), 8.80 (d, J = 2.5 Hz, 1H), 8.19 (d, J = 8.6 Hz, 1H), 7.77 (dd, J = 8.6, 7.0 Hz, 1H), 7.52-7.44 (m, | DMSO | >98 | Method R5 | | | |
| | | | | | 1H), 7.40 (dd, | | | | | | |
| | | | | | J = 11.4, 8.4 Hz, | | | | | | |
| | | | | | 1H), 7.29 (dd, J = | | | | | | |
| | | | | | 8.2, 4.7 Hz, 1H), | | | | | | |
| | | | | | 3.94 (s, 3H), 3.20 | | | | | | |
| | | | | | (d, J = 4.4 Hz, 3H). | | | | | | |
| | | | | | 1H of HCl was | | | | | | |
| | | | | | not observed. | | | | | | |
| 1010 | 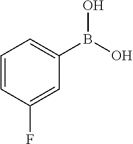 | 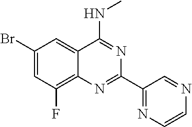 [SMILES: CNc1nc(-c2cnccn2)nc2c(F)cc(Br)cc12] | 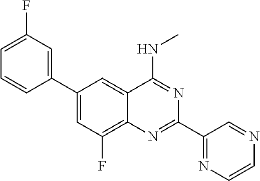 [SMILES: CNc1nc(-c2cnccn2)nc2c(F)cc(-c3cccc(F)c3)cc12] | HCl | 1H NMR (400 MHz, DMSO) δ 9.66 (d, J = 1.5 Hz, 1H), 8.91 (d, J = 4.2 Hz, 1H), 8.85 (dd, J = 2.4, 1.5 Hz, 1H), 8.79 (d, J = 2.4 Hz, 1H), 8.54 (d, J = 1.8 Hz, 1H), 8.18 (dd, J = 12.0, 1.8 Hz, 1H), 7.83-7.75 (m, | DMSO | >98 | Method R5 | | | |
| | | | | | 2H), 7.60 (td, J = | | | | | | |
| | | | | | 8.2, 8.2, 6.2 Hz, | | | | | | |
| | | | | | 1H), 7.30 (td, J = | | | | | | |
| | | | | | 8.8, 8.6, 2.4 Hz, | | | | | | |
| | | | | | 1H), 3.21 (d, J = | | | | | | |
| | | | | | 4.4 Hz, 3H). 1H of | | | | | | |
| | | | | | HCl was not | | | | | | |
| | | | | | observed. | | | | | | |
| 1011 | 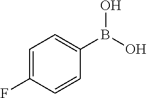 | 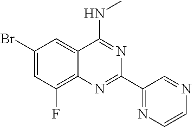 [SMILES: CNc1nc(-c2cnccn2)nc2c(F)cc(Br)cc12] | 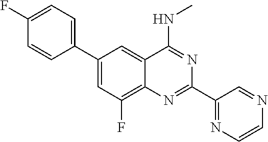 [SMILES: CNc1nc(-c2cnccn2)nc2c(F)cc(-c3ccc(F)cc3)cc12] | 2HCl | 1H NMR (400 MHz, DMSO) δ 9.65 (d, J = 1.5 Hz, 1H), 8.89 (d, J = 5.0 Hz, 1H), 8.84 (dd, J = 2.5, 1.5 Hz, 1H), 8.78 (d, J = 2.5 Hz, 1H), 8.47 (s, 1H), 8.11 (dd, | DMSO | >98 | Method R5 | | | |
| | | | | | J = 12.0, 1.8 Hz, | | | | | | |
| | | | | | 1H), 8.01-7.90 | | | | | | |
| | | | | | (m, 2H), 7.47- | | | | | | |
| | | | | | 7.34 (m, 2H), 3.20 | | | | | | |
| | | | | | (d, J = 4.4 Hz, | | | | | | |
| | | | | | 3H). 1H of HCl was | | | | | | |
| | | | | | not observed. | | | | | | |
| 1012 | 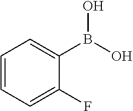 | 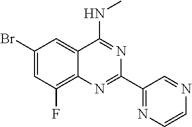 [SMILES: CNc1nc(-c2cnccn2)nc2c(F)cc(Br)cc12] | 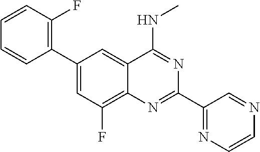 [SMILES: CNc1nc(-c2cnccn2)nc2c(F)cc(-c3ccccc3F)cc12] | 2HCl | 1H NMR (400 MHz, DMSO) δ 9.67 (d, J = 1.5 Hz, 1H), 8.99 (d, J = 4.6 Hz, 1H), 8.86 (dd, J = 2.4, 1.4 Hz, 1H), 8.81 (d, J = 2.5 Hz, 1H), 8.36 (d, J = 1.6 Hz, 1H), 7.94 (dd, J = | DMSO | >98 | Method R5 | | | |
| | | | | | 13.0, 2.2 Hz, 1H), | | | | | | |
| | | | | | 7.72 (td, J = 7.9, | | | | | | |
| | | | | | 7.8, 1.7 Hz, 1H), | | | | | | |
| | | | | | 7.53 (ddd, J = 9.5, | | | | | | |
| | | | | | 4.6, 2.0 Hz, 1H), | | | | | | |
| | | | | | 7.49-7.31 (m, | | | | | | |
| | | | | | 2H), 3.19 (d, J = 4.4 | | | | | | |
| | | | | | Hz, 3H). 1H of | | | | | | |
| | | | | | HCl was not | | | | | | |
| | | | | | observed. | | | | | | |
| 1013 | 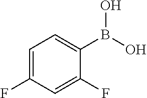 [SMILES: OB(O)c1ccc(F)cc1F] | 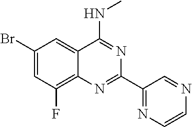 [SMILES: CNc1nc(-c2cnccn2)nc2c(F)cc(Br)cc12] | 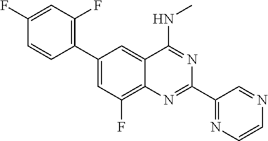 [SMILES: CNc1nc(-c2cnccn2)nc2c(F)cc(-c3ccc(F)cc3F)cc12] | HCl | 1H NMR (400 MHz, DMSO) δ 9.66 (d, J = 1.5 Hz, 1H), 8.94-8.82 (m, 2H), 8.79 (d, J = 2.5 Hz, 1H), 8.38- 8.22 (m, 1H), 7.91 (dt, J = 11.5, 1.7, 1.7 Hz, 1H), 7.77 (td, J = 8.9, | DMSO | >98 | Method R5 | | | |
| | | | | | 8.9, 6.5 Hz, 1H), | | | | | | |
| | | | | | 7.49 (ddd, J = | | | | | | |
| | | | | | 11.5, 9.3, 2.6 Hz, | | | | | | |
| | | | | | 1H), 7.32 (td, J = | | | | | | |
| | | | | | 8.6, 8.6, 2.3 Hz, | | | | | | |
| | | | | | 1H), 3.18 (d, J = | | | | | | |
| | | | | | 4.4 Hz, 3H). | | | | | | |
| | | | | | 1H of HCl was | | | | | | |
| | | | | | not observed. | | | | | | |
| 1014 | 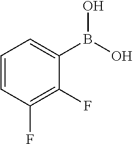 [SMILES: OB(O)c1cccc(F)c1F] | 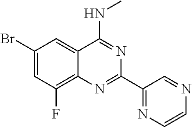 [SMILES: CNc1nc(-c2cnccn2)nc2c(F)cc(Br)cc12] | 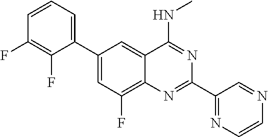 [SMILES: CNc1nc(-c2cnccn2)nc2c(F)cc(-c3cccc(F)c3F)cc12] | HCl | 1H NMR (400 MHz, DMSO) δ 9.66 (d, J = 1.5 Hz, 1H), 8.85 (dd, J - 2.4, 1.5 Hz, 2H), 8.79 (d, J = 2.4 Hz, 1H), 8.37 (s, 1H), 7.96 (d, J = 11.4 Hz, 1H), 7.67- | DMSO | >98 | Method R5 | | | |
| | | | | | 7.47 (m, 2H), | | | | | | |
| | | | | | 7.41 (s, 1H), 3.18 | | | | | | |
| | | | | | (d, J = 4.4 Hz, 3H). | | | | | | |
| | | | | | 1H of HCl was | | | | | | |
| | | | | | not observed. | | | | | | |
| 1015 | 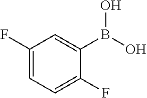 [SMILES: OB(O)c1cc(F)ccc1F] | 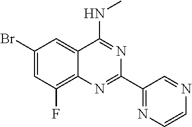 [SMILES: CNc1nc(-c2cnccn2)nc2c(F)cc(Br)cc12] | 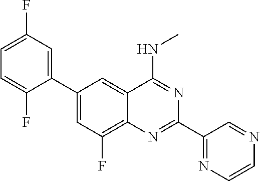 [SMILES: CNc1nc(-c2cnccn2)nc2c(F)cc(-c3cc(F)ccc3F)cc12] | HCl | 1H NMR (400 MHz, DMSO) δ 9.66 (d, J = 1.5 Hz, 1H), 8.90 (d, J = 4.0 Hz, 1H), 8.85 (dd, J = 2.5, 1.5 Hz, 1H), 8.79 (d, J = 2.5 Hz, 1H), 8.38 (s, 1H), 7.96 (dt, J = 11.5, 1.7, 1.7 Hz, 1H), 7.63 (ddd, J = 9.3, 6.1, 3.2 | DMSO | >98 | Method R5 | | | |
| | | | | | Hz, 1H), 7.53-7.43 | | | | | | |
| | | | | | (m, 1H), 7.42- | | | | | | |
| | | | | | 7.31 (m, 1H), 3.19 | | | | | | |
| | | | | | (d, J = 4.4 Hz, | | | | | | |
| | | | | | 3H). 1H of HCl was | | | | | | |
| | | | | | not observed. | | | | | | |
| 1016 | 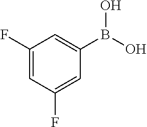 [SMILES: OB(O)c1cc(F)cc(F)c1] | 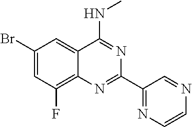 [SMILES: CNc1nc(-c2cnccn2)nc2c(F)cc(Br)cc12] | 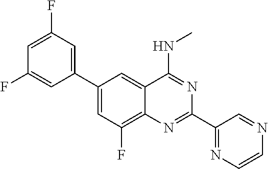 [SMILES: CNc1nc(-c2cnccn2)nc2c(F)cc(-c3cc(F)cc(F)c3)cc12] | 2HCl | 1H NMR (400 MHz, DMSO) δ 9.66 (d, J = 1.5 Hz, 1H), 8.99 (d, J = 5.2 Hz, 1H), 8.85 (t, J = 2.0, 2.0 Hz, 1H), 8.80 (d, J = 2.5 Hz, 1H), 8.59 (d, J = 1.9 Hz, 1H), 8.24 (dd, J = 11.9, 1.8 Hz, 1H), | DMSO | >98 | Method R3 | | | |
| | | | | | 7.84-7.56 (m, | | | | | | |
| | | | | | 2H), 7.35 (tt, J = | | | | | | |
| | | | | | 9.2, 9.2, 2.4, 2.4 | | | | | | |
| | | | | | Hz, 1H), 3.21 (d, | | | | | | |
| | | | | | J = 4.4 Hz, 3H). | | | | | | |
| | | | | | 1H of HCl was | | | | | | |
| | | | | | not observed. | | | | | | |
| 1017 | 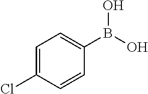 | 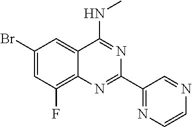 [SMILES: CNc1nc(-c2cnccn2)nc2c(F)cc(Br)cc12] | 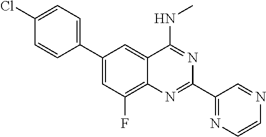 [SMILES: CNc1nc(-c2cnccn2)nc2c(F)cc(-c3ccc(Cl)cc3)cc12] | HCl | 1H NMR (400 MHz, DMSO) δ 9.66 (d, J = 1.5 Hz, 1H), 8.98 (d, J = 5.2 Hz, 1H), 8.85 (t, J = 1.8, 1.8 Hz, 1H), 8.79 (d, J = 2.3 Hz, 1H), 8.52 (d, J = 1.9 Hz, | DMSO | >98 | Method R3 Temper- ature at 80° C. | | | |
| | | | | | 1H), 8.14 (dd, J = | | | | | | |
| | | | | | 11.9, 1.8 Hz, 1H), | | | | | | |
| | | | | | 8.00-7.88 (m, | | | | | | |
| | | | | | 2H), 7.68-7.53 (m, | | | | | | |
| | | | | | 2H), 3.21 (d, | | | | | | |
| | | | | | J = 4.2 Hz, 3H). | | | | | | |
| | | | | | 1H of HCl was not | | | | | | |
| | | | | | observed. | | | | | | |
| 1018 | 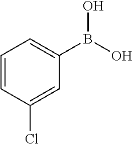 | 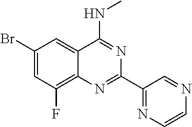 [SMILES: CNc1nc(-c2cnccn2)nc2c(F)cc(Br)cc12] | 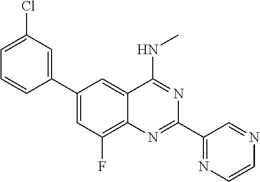 [SMILES: CNc1nc(-c2cnccn2)nc2c(F)cc(-c3cccc(Cl)c3)cc12] | HCl | 1H NMR (400 MHz, DMSO) δ 9.66 (d, J = 1.5 Hz, 1H), 8.97 (d, J = 4.6 Hz, 1H), 8.85 (dd, J = 2.4, 1.5 Hz, 1H), 8.79 (d, J = 2.5 Hz, 1H), 8.54 (d, J = 1.8 Hz, 1H), 8.18 (dd, J = 12.0, 1.8 Hz, 1H), 8.01 (t, J = 1.9, | DMSO | >98 | Method R3 Temper- ature at 80° C. | | | |
| | | | | | 1.9 Hz, 1H), 7.89 | | | | | | |
| | | | | | (dt, J = 7.7, 1.4, | | | | | | |
| | | | | | 1.4 Hz, 1H), 7.65- | | | | | | |
| | | | | | 7.55 (m, 1H), | | | | | | |
| | | | | | 7.55-7.47 (m, 1H), | | | | | | |
| | | | | | 3.21 (d, J = | | | | | | |
| | | | | | 4.4 Hz, 3H). 1H | | | | | | |
| | | | | | of HCl was | | | | | | |
| | | | | | not observed. | | | | | | |
| 1019 | 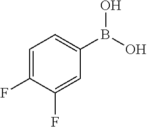 [SMILES: OB(O)c1ccc(F)c(F)c1] | 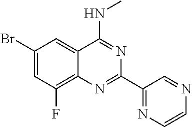 [SMILES: CNc1nc(-c2cnccn2)nc2c(F)cc(Br)cc12] | 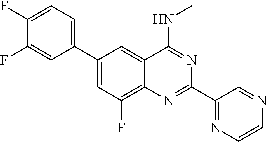 [SMILES: CNc1nc(-c2cnccn2)nc2c(F)cc(-c3ccc(F)c(F)c3)cc12] | HCl | 1H NMR (400 MHz, DMSO) δ 9.74- 9.59 (m, 1H), 8.84 (dd, J = 2.5, 1.5 Hz, 2H), 8.78 (d, J = 2.5 Hz, 1H), 8.55-8.36 (m, 1H), 8.23-8.08 (m, 1H), 8.08-7.87 | DMSO | >98 | Method R3 | | | |
| | | | | | (m, 1H), 7.76 | | | | | | |
| | | | | | (brs, 1H), 7.62 (dt, | | | | | | |
| | | | | | J = 10.4, 8.6, | | | | | | |
| | | | | | 8.6 Hz, 1H), 3.21 | | | | | | |
| | | | | | (d, J = 4.3 Hz, 3H). | | | | | | |
| | | | | | 1H of HCl was | | | | | | |
| | | | | | not observed. | | | | | | |
| 1020 | 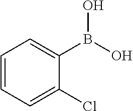 | 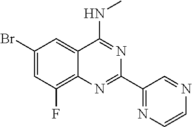 [SMILES: CNc1nc(-c2cnccn2)nc2c(F)cc(Br)cc12] | 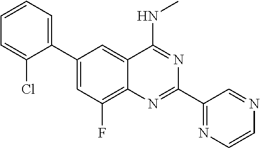 [SMILES: CNc1nc(-c2cnccn2)nc2c(F)cc(-c3ccccc3Cl)cc12] | HCl | 1H NMR (400 MHz, DMSO) δ 9.66 (d( J = 1.5 Hz, 1H), 8.87-8.77 (m, 3H), 8.26-8.14 (m, 1H), 7.84 (dd, J = 11.3, 1.7 Hz, 1H), 7.70-7.62 (m, 1H), 7.62-7.56 (m, 1H), 7.56- | DMSO | >98 | Method R3 Temper- ature at 80° C. | | | |
| | | | | | 7.45 (m, 2H), 3.16 | | | | | | |
| | | | | | (d, J = 4.3 Hz, | | | | | | |
| | | | | | 3H). 1H of HCl was | | | | | | |
| | | | | | not observed. | | | | | | |
| 1021 | 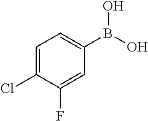 [SMILES: OB(O)c1ccc(Cl)c(F)c1] | 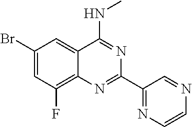 [SMILES: CNc1nc(-c2cnccn2)nc2c(F)cc(Br)cc12] | 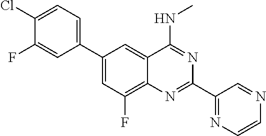 [SMILES: CNc1nc(-c2cnccn2)nc2c(F)cc(-c3ccc(Cl)c(F)c3)cc12] | HCl | 1H NMR (400 MHz, DMSO) δ 9.65 (d, J = 1.5 Hz, 1H), 8.84 (dd, J = 2.5, 1.6 Hz, 2H), 8.78 (d, J = 2.5 Hz, 1H), 8.54 (s, 1H), 8.20 (dd, J = 11.9, 1.8 Hz, 1H), | DMSO | >98 | Method R3 Temper- ature at 80° C. | | | |
| | | | | | 8.01 (dd, J = | | | | | | |
| | | | | | 11.2, 1.9 Hz, 1H), | | | | | | |
| | | | | | 7.88-7.71 (m, | | | | | | |
| | | | | | 2H), 3.20 (d, J = | | | | | | |
| | | | | | 4.4 Hz, 3H). | | | | | | |
| | | | | | 1H of HCl was | | | | | | |
| | | | | | not observed. | | | | | | |
| 1022 | 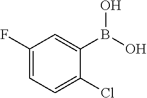 [SMILES: OB(O)c1cc(F)ccc1Cl] | 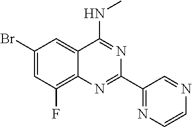 [SMILES: CNc1nc(-c2cnccn2)nc2c(F)cc(Br)cc12] | 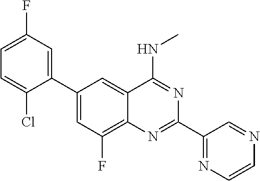 [SMILES: CNc1nc(-c2cnccn2)nc2c(F)cc(-c3cc(F)ccc3Cl)cc12] | HCl | 1H NMR (400 MHz, DMSO) δ 9.66 (d, J = 1.4 Hz, 1H), 8.89-8.73 (m, 3H), 8.31-8.20 (m, 1H), 7.88 (dd, J = 11.2,1.7 Hz, 1H), 7.71 (dd, J = 8.9, 5.2 Hz, 1H), 7.54 (dd, J = 9.2, 3.0 Hz, 1H), 7.40 (td, J = 8.5, 8.5, | DMSO | >98 | Method R3 Temper- ature at 80° C. | | | |
| | | | | | 3.1 Hz, 1H), 3.17 | | | | | | |
| | | | | | (d, J = 4.4 Hz, | | | | | | |
| | | | | | 3H). 1H of HCl was | | | | | | |
| | | | | | not observed. | | | | | | |
| 1023 | 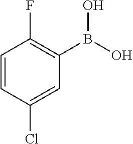 [SMILES: OB(O)c1cc(Cl)ccc1F] | 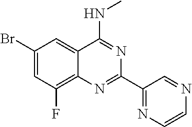 [SMILES: CNc1nc(-c2cnccn2)nc2c(F)cc(Br)cc12] | 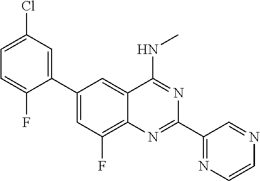 [SMILES: CNc1nc(-c2cnccn2)nc2c(F)cc(-c3cc(Cl)ccc3F)cc12] | HCl | 11H NMR (400 MHz, DMSO) δ 9.66 (d, J = 1.5 Hz, 1H), 8.85 (dd,J = 2.4, 1.5 Hz, 2H), 8.79 (d, J = 2.4 Hz, 1H), 8.36 (d, J = 1.7 Hz, 1H), 7.96 (dt, J = 11.5, 1.8, 1.8 Hz, 1H), 7.82 (dd, J = 6.8, 2.7 Hz, 1H), 7.59 | DMSO | >98 | Method R3 Temper- ature at 80° C. | | | |
| | | | | | (ddd, J = 8.9, 4.2, | | | | | | |
| | | | | | 2.6 Hz, 1H), 7.53- | | | | | | |
| | | | | | 7.43 (m, 1H), 3.18 | | | | | | |
| | | | | | (d, J = 4.4 Hz, | | | | | | |
| | | | | | 3H). 1H of HCl was | | | | | | |
| | | | | | not observed. | | | | | | |
| 1024 | 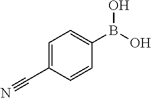 [SMILES: N#Cc1ccc(B(O)O)cc1] | 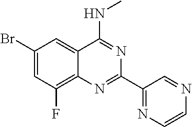 [SMILES: CNc1nc(-c2cnccn2)nc2c(F)cc(Br)cc12] | 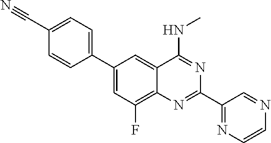 [SMILES: CNc1nc(-c2cnccn2)nc2c(F)cc(-c3ccc(C#N)cc3)cc12] | HCl | 1H NMR (400 MHz, DMSO) δ 9.66 (d, J = 1.5 Hz, 1H), 8.95 (d, J = 5.2 Hz, 1H), 8.85 (dd, J = 2.4, 1.5 Hz, 1H), 8.79 (d, J = 2.4 Hz, 1H), 8.61 (d,J = 1.8 Hz, 1H), | DMSO | >98 | Method R3 | | | |
| | | | | | 8.22 (dd, J = | | | | | | |
| | | | | | 11.9, 1.8 Hz, 1H), | | | | | | |
| | | | | | 8.15-8.08 (m, | | | | | | |
| | | | | | 2H), 8.08-7.99 (m, | | | | | | |
| | | | | | 2H), 3.21 (d, | | | | | | |
| | | | | | J = 4.4 Hz, 3H). 1H | | | | | | |
| | | | | | of HCl was not | | | | | | |
| | | | | | observed. | | | | | | |
| 1025 | 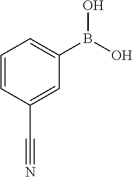 [SMILES: N#Cc1cccc(B(O)O)c1] | 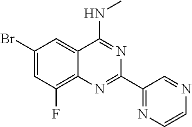 [SMILES: CNc1nc(-c2cnccn2)nc2c(F)cc(Br)cc12] | 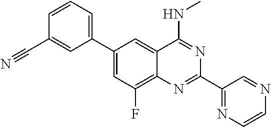 [SMILES: CNc1nc(-c2cnccn2)nc2c(F)cc(-c3cccc(C#N)c3)cc12] | HCl | 1H NMR (400 MHz, DMSO) δ 9.66 (d, J = 1.5 Hz, 1H), 8.92 (d, J = 5.3 Hz, 1H),8.85 (dd, J = 2.4, 1.5 Hz, 1H), 8.79 (d, J = 2.4 Hz, 1H), 8.58 (d, J = 1.9 Hz, 1H), 8.40 (t, J = 1.7, 1.7 Hz, 1H), 8.31- | DMSO | >98 | Method R3 | | | |
| | | | | | 8.18 (m, 2H), | | | | | | |
| | | | | | 7.93 (dt, J = 7.6, 1.3, | | | | | | |
| | | | | | 1.3 Hz, 1H), | | | | | | |
| | | | | | 7.77 (t, J = 7.8, 7.8 | | | | | | |
| | | | | | Hz, 1H), 3.22 | | | | | | |
| | | | | | (d, J = 4.4 Hz, 3H). | | | | | | |
| | | | | | 1H of HCl was | | | | | | |
| | | | | | not observed. | | | | | | |
| 1026 | 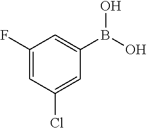 [SMILES: OB(O)c1cc(F)cc(Cl)c1] | 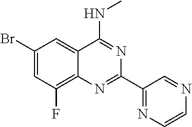 [SMILES: CNc1nc(-c2cnccn2)nc2c(F)cc(Br)cc12] | 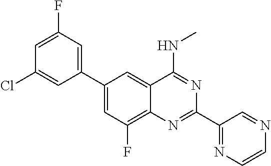 [SMILES: CNc1nc(-c2cnccn2)nc2c(F)cc(-c3cc(F)cc(Cl)c3)cc12] | HCl | 1H NMR (400 MHz, DMSO) δ 9.66 (d, J = 1.4 Hz, 1H), 8.91-8.82 (m, 2H), 8.78 (d, J = 2.5 Hz, 1H), 8.60- 8.49 (m, 1H), 8.23 (dd, J = 11.9, 1.8 Hz, 1H), 7.91 (t, J = 1.7,1.7 Hz, 1H), 7.82 (dt, J = | DMSO | >98 | Method R3 Temper- ature at 80° C. | | | |
| | | | | | 10.2, 2.0, 2.0 Hz, | | | | | | |
| | | | | | 1H), 7.54 (dt, J = | | | | | | |
| | | | | | 8.5, 2.1, 2.1 Hz, | | | | | | |
| | | | | | 1H), 3.21 (d, J = | | | | | | |
| | | | | | 4.4 Hz, 3H). 1H of | | | | | | |
| | | | | | HCl was not | | | | | | |
| | | | | | observed. | | | | | | |
| 1027 | 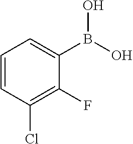 [SMILES: OB(O)c1cccc(Cl)c1F] | 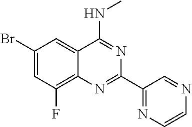 [SMILES: CNc1nc(-c2cnccn2)nc2c(F)cc(Br)cc12] | 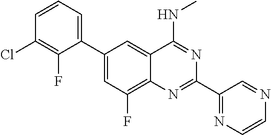 [SMILES: CNc1nc(-c2cnccn2)nc2c(F)cc(-c3cccc(Cl)c3F)cc12] | HCl | 1H NMR (400 MHz, DMSO) δ 9.66 (d, J = 1.5 Hz, 1H), 8.97 (d, J = 4.8 Hz, 1H), 8.86 (dd, J = 2.4, 1.5 Hz, 1H), 8.80 (d, J = 2.5 Hz, 1H), 8.38 (s, 1H), 7.98 (dt, | DMSO | >98 | Method R3 Temper- ature at 80° C. | | | |
| | | | | | J = 11.2, 1.7, 1.7 | | | | | | |
| | | | | | Hz, 1H), 7.79-7.63 | | | | | | |
| | | | | | (m, 2H), 7.43 | | | | | | |
| | | | | | (td, J = 8.0, 8.0, 0.9 | | | | | | |
| | | | | | Hz, 1H), 3.19 | | | | | | |
| | | | | | (d, J = 4.4 Hz, 3H). | | | | | | |
| | | | | | 1H of HCl was | | | | | | |
| | | | | | not observed. | | | | | | |
| 1028 | 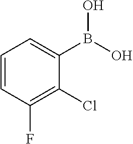 [SMILES: OB(O)c1cccc(F)c1Cl] | 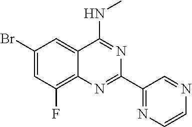 [SMILES: CNc1nc(-c2cnccn2)nc2c(F)cc(Br)cc12] | 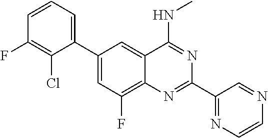 [SMILES: CNc1nc(-c2cnccn2)nc2c(F)cc(-c3cccc(F)c3Cl)cc12] | HCl | 1H NMR (400 MHz, DMSO) δ 9.66 (d, J = 1.5 Hz, 1H), 8.89-8.75 (m, 3H), 8.24 (d, J = 1.7 Hz, 1H), 7.88 (dd, J = 11.2,1.7 Hz, 1H), 7.63- 7.49 (m, 2H), 7.48- | DMSO | >98 | Method R3 Temper- ature at 80° C. | | | |
| | | | | | 7.39 (m, 1H), | | | | | | |
| | | | | | 3.16 (d, J = 4.3 Hz, | | | | | | |
| | | | | | 3H). 1H of HCl | | | | | | |
| | | | | | was not observed. | | | | | | |
| 1029 | 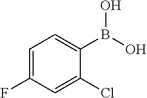 [SMILES: OB(O)c1ccc(F)cc1Cl] | 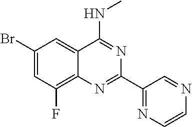 [SMILES: CNc1nc(-c2cnccn2)nc2c(F)cc(Br)cc12] | 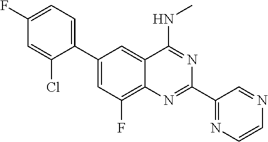 [SMILES: CNc1nc(-c2cnccn2)nc2c(F)cc(-c3ccc(F)cc3Cl)cc12] | HCl | 1H NMR (400 MHz, DMSO) δ 9.66 (d, J = 1.5 Hz, 1H), 8.85 (dd, J = 2.4, 1.5 Hz, 1H), 8.79 (d, J = 2.5 Hz, 2H), 8.25-8.12 (m, 1H), 7.83 (dd, J = 11.3, 1.7 | DMSO | >98 | Method R3 Temper- ature at 80° C. | | | |
| | | | | | Hz, 1H), 7.73- | | | | | | |
| | | | | | 7.62 (m, 2H), 7.43 | | | | | | |
| | | | | | (td, J = 8.5, 8.4, | | | | | | |
| | | | | | 2.6 Hz, 1H), 3.16 | | | | | | |
| | | | | | (d, J = 4.4 Hz, | | | | | | |
| | | | | | 3H). 1H of HCl was | | | | | | |
| | | | | | not observed. | | | | | | |
| 1030 | 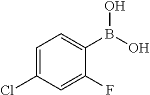 [SMILES: OB(O)c1ccc(Cl)cc1F] | 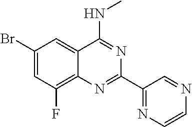 [SMILES: CNc1nc(-c2cnccn2)nc2c(F)cc(Br)cc12] | 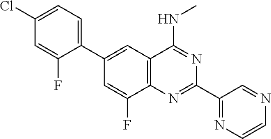 [SMILES: CNc1nc(-c2cnccn2)nc2c(F)cc(-c3ccc(Cl)cc3F)cc12] | HCl | 1H NMR (400 MHz, DMSO) δ 9.66 (d, J = 1.4 Hz, 1H), 8.93-8.82 (m, 2H), 8.79 (d, J = 2.5 Hz, 1H), 8.34 (S, 1H), 7.92 (dd, J = 13.0, 1.9 Hz, 1H), 7.75 (t, J = 8.5, 8.5 Hz, 1H), | DMSO | >98 | Method R3 Temper- ature at 80° C. | | | |
| | | | | | 7.68 (dd, J = 10.7, | | | | | | |
| | | | | | 2.1 Hz, 1H), | | | | | | |
| | | | | | 7.51 (dd, J = 8.3, | | | | | | |
| | | | | | 2.0 Hz, 1H), 3.18 | | | | | | |
| | | | | | (d, J = 4.4 | | | | | | |
| | | | | | Hz, 3H). | | | | | | |
| | | | | | 1H of HCl was | | | | | | |
| | | | | | not observed. | | | | | | |
| 1031 | 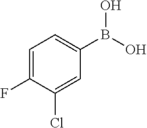 [SMILES: OB(O)c1ccc(F)c(Cl)c1] | 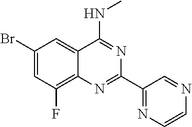 [SMILES: CNc1nc(-c2cnccn2)nc2c(F)cc(Br)cc12] | 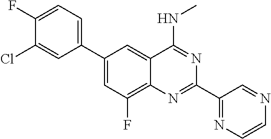 [SMILES: CNc1nc(-c2cnccn2)nc2c(F)cc(-c3ccc(F)c(Cl)c3)cc12] | HCl | 1H NMR (400 MHz, DMSO) δ 9.65 (d, J = 1.5 Hz, 1H), 8.94 (d, J = 5.1 Hz, 1H), 8.85 (t, J = 2.0, 2.0 Hz, 1H), 8.79 (d, J = 2.5 Hz, 1H), 8.51 (d, J = 2.0 Hz, 1H), | DMSO | >98 | Method R3 Temper- ature at 80° C. | | | |
| | | | | | 8.24-8.10 (m, | | | | | | |
| | | | | | 2H), 8.01-7.86 (m, | | | | | | |
| | | | | | 1H), 7.63 (t, J = | | | | | | |
| | | | | | 9.0, 9.0 Hz, 1H), | | | | | | |
| | | | | | 3.21 (d, J = 4.4 | | | | | | |
| | | | | | Hz,3H). 1H of | | | | | | |
| | | | | | HCl was not | | | | | | |
| | | | | | observed. | | | | | | |

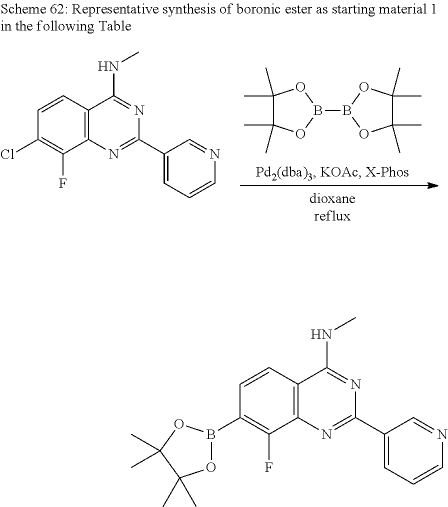

### 8-fluoro-N-methyl-2-(pyridin-3-yl)-7-(4,4,5,5-tetramethyl-1,3,2-dioxaborolan-2-yl)quinazolin-4-amine

Tris(dibenzylideneacetone)dipalladium (0) (95 mg, 0.104 mmol) was dissolved in dioxane (30 ml) under N₂. 2-(Dicyclohexylphosphino)-2′,4′,6′-triisopropylbiphenyl (X-phos) (198 mg, 0.416 mmol), Potassium acetate (612 mg, 6.23 mmol), Bis(pinacolato)diboron (792 mg, 3.12 mmol) and 7-chloro-8-fluoro-N-methyl-2-(pyridin-3-yl)quinazolin-4-amine (600 mg, 2.078 mmol) were added at RT. The mixture was refluxed for 2 hr. H₂O and ethyl acetate were added. The organic phase was extracted with EA, dried over Na₂SO₄. Filtration and concentration gave the solid. The solid was trituated in ethyl acetate/Hexane (1/1, 20/20 ml). The solid was collected and washed with Hexane, dried in vacuo.

620 mg was obtained (78% yield).

¹H NMR (400 MHz, DMSO) δ 9.62 (dd, J=2.2, 0.9 Hz, 1H), 8.75 (dt, J=7.9, 2.0, 2.0 Hz, 1H), 8.73-8.60 (m, 2H), 8.02 (d, J=8.4 Hz, 1H), 7.62 (dd, J=8.3, 5.1 Hz, 1H), 7.60-7.51 (m, 1H), 3.15 (d, J=4.4 Hz, 3H), 1.35 (s, 12H).

Note: In case of using the excess diboron and 10 mol % Pd₂(dba)₃, hydrolysis proceeded.

### 8-fluoro-4-(methylamino)-2-(pyridin-3-yl)quinazolin-7-ylboronic acid

¹H NMR (400 MHz, DMSO) δ 9.63 (dd, J=1.9, 0.9 Hz, 1H), 8.76 (dt, J=7.9, 1.9, 1.9 Hz, 1H), 8.69 (dd, J=4.8, 1.8 Hz, 1H), 8.56 (d, J=5.1 Hz, 1H), 8.53 (s, 2H), 7.97 (d, J=8.2 Hz, 1H), 7.55 (ddd, J=8.0, 4.8, 2.7 Hz, 2H), 3.16 (d, J=4.3 Hz, 3H).

TABLE 35

| | | | | | | | | Method | | Reten- | |
|---|---|---|---|---|---|---|---|---|---|---|---|
| | Starting | Starting | | Salt | | ¹H NMR | Purity | of | | tion | LCMS |
| Number | Material 1 | Material 2 | Product | type | ¹H NMR | Solvent | percent | Coupling | LCMS | Time | Method |
| 1032 | 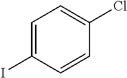 | 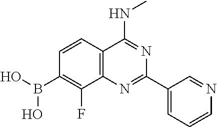 [SMILES: CNc1nc(-c2cccnc2)nc2c(F)c(B(O)O)ccc12] | 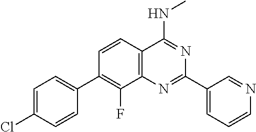 [SMILES: CNc1nc(-c2cccnc2)nc2c(F)c(-c3ccc(Cl)cc3)ccc12] | 2HCl | ¹H NMR (400 MHz, DMSO) δ 9.69 (d, J = 2.0 Hz, 1H), 9.27 (d, J = 7.9 Hz, 1H), 8.95 (t, J = 4.8, 4.8 Hz, 2H), 8.20 (d, J = 8.6 Hz, 1H), 8.03 (t, J = 6.8, 6.8 Hz, 1H), 7.81-7.67 (m, 3H), 7.67-7.59 (m, 2H), 3.21 (d, J = 4.4 Hz, 3H). 1H of 2HCl was not observed. | DMSO | >98 | Method R3 Temperature at 80° C. | | | |
| 1033 | 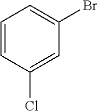 | 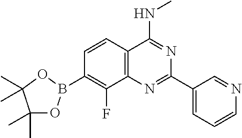 [SMILES: CNc1nc(-c2cccnc2)nc2c(F)c(B3OC(C)(C)C(C)(C)O3)ccc12] | 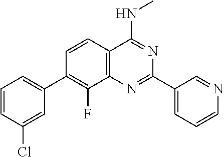 [SMILES: CNc1nc(-c2cccnc2)nc2c(F)c(-c3cccc(Cl)c3)ccc12] | HCl | ¹H NMR (400 MHz, DMSO) δ 9.67 (d, J = 2.0 Hz, 1H), 8.96 (d, J = 7.9 Hz, 1H), 8.86- 8.73 (m, 2H), 8.16 (d, J = 8.6 Hz, 1H), 7.79 (d, 7 = 2.0 Hz, 1H), 7.71 (t, J = 8.0, 8.0 Hz, 3H), 7.65-7.49 (m, 2H), 3.20 (d, J = 4.2 Hz, 3H). 1H of HCl was not observed. | DMSO | >98 | Method R3 Temperature at 80° C. | | | |
| 1034 | 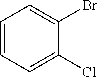 | 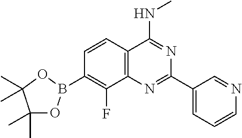 [SMILES: CNc1nc(-c2cccnc2)nc2c(F)c(B3OC(C)(C)C(C)(C)O3)ccc12] | 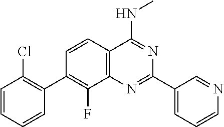 [SMILES: CNc1nc(-c2cccnc2)nc2c(F)c(-c3ccccc3Cl)ccc12] | HCl | ¹H NMR (400 MHz, DMSO) δ 9.68 (d, J = 2.0 Hz, 1H), 9.11 (dt, J = 8.0, 1.7, 1.7 Hz, 1H), 8.86 (dd, J = 5.1, 1.6 Hz, 2H), 8.17 (d, J = 8.5 Hz, 1H), 7.87 (dd, J = 8.0, 5.1 Hz, 1H), 7.77- 7.61 (m, 1H), 7.61-7.25 (m, 4H), 3.21 (d, J = 4.4 Hz, 3H). 1H of HC1 was not observed. | DMSO | >98 | Method R3 Temperature at 80° C. | | | |
| 1035 | 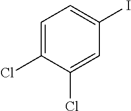 | 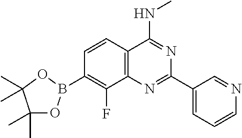 [SMILES: CNc1nc(-c2cccnc2)nc2c(F)c(B3OC(C)(C)C(C)(C)O3)ccc12] | 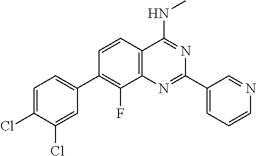 [SMILES: CNc1nc(-c2cccnc2)nc2c(F)c(-c3ccc(Cl)c(Cl)c3)ccc12] | 3HCl | ¹H NMR (400 MHz, DMSO) δ 9.69 (d, J = 2.0 Hz, 1H), 9.29 (d, J = 7.2 Hz, 1H),9.05- 8.88 (m, 2H), 8.22 (d, J = 8.6 Hz, 1H), 8.06 (dd, J = 8.1, 5.3 Hz, 1H), 8.01 (d, J = 2.1 Hz, 1H), 7.88-7.81 (m, 1H), 7.76 (td, J = 9.4, 9.1, 7.1 Hz, 2H), 3.21 (d, J = 4.3 Hz, 3H). 1H of 3HCl was not observed. | DMSO | >98 | Method R3 Temperature at 80° C. | | | |
| 1036 | 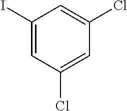 | 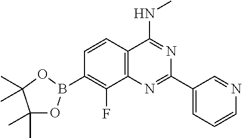 [SMILES: CNc1nc(-c2cccnc2)nc2c(F)c(B3OC(C)(C)C(C)(C)O3)ccc12] | 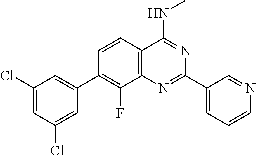 [SMILES: CNc1nc(-c2cccnc2)nc2c(F)c(-c3cc(Cl)cc(Cl)c3)ccc12] | 2HCl | ¹H NMR (400 MHz, DMSO) δ 9.66 (d, J = 2.1 Hz, 1H), 8.89 (d, J = 8.1 Hz, 1H), 8.76 (d, J = 5.5 Hz, 2H), 8.16 (d, J = 8.7 Hz, 1H), 7.84-7.59 (m, 5H), 3.19 (d, J = 4.3 Hz, 3H). 1H of 2HCl was not observed. | DMSO | >98 | Method R3 Temperature at 80° C. | | | |

TABLE 36

| | | | | | | | | Method | | Reten- | |
|---|---|---|---|---|---|---|---|---|---|---|---|
| | Starting | Starting | | Salt | | ¹H NMR | Purity | of | | tion | LCMS |
| Number | Material 1 | Material 2 | Product | type | ¹H NMR | Solvent | percent | Coupling | LCMS | Time | Method |
| 1037 | 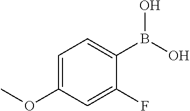 | 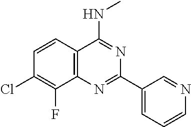 [SMILES: CNc1nc(-c2cccnc2)nc2c(F)c(Cl)ccc12] | 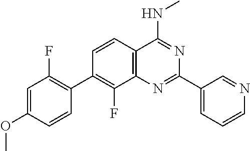 [SMILES: CNc1nc(-c2cccnc2)nc2c(F)c(-c3ccc(OC)cc3F)ccc12] | 2HCl | 1H NMR (400 MHz, DMSO) δ 9.69 (d, J = 1.9 Hz, 1H), 9.29 (d, J = 8.1 Hz, 1H), 9.02-8.91 (m, 2H), 8.19 (d, J = 8.6 Hz, 1H), 8.05 (t, J = 7.1, 7.1 Hz, 1H), 7.60 (dd, J = 8.4, 6.8 | DMSO | >98 | Method R5 | | | |
| | | | | | Hz, 1H), 7.53 (t, | | | | | | |
| | | | | | J = 8.7, 8.7 Hz, | | | | | | |
| | | | | | 1H), 7.05 (dd, J = | | | | | | |
| | | | | | 12.3, 2.5 Hz, | | | | | | |
| | | | | | 1H), 6.99 (dd, J = | | | | | | |
| | | | | | 8.6, 2.5 Hz, 1H), | | | | | | |
| | | | | | 3.89 (s, 3H), 3.21 | | | | | | |
| | | | | | (d, J = 4.3 Hz, | | | | | | |
| | | | | | 3H). 1H of 2HCl | | | | | | |
| | | | | | was not observed. | | | | | | |
| 1038 | 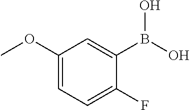 | 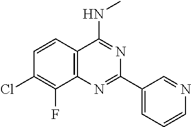 [SMILES: CNc1nc(-c2cccnc2)nc2c(F)c(Cl)ccc12] | 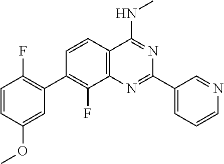 [SMILES: CNc1nc(-c2cccnc2)nc2c(F)c(-c3cc(OC)ccc3F)ccc12] | 2HCl | 1H NMR (400 MHz, DMSO) δ 9.69 (d, J = 1.9 Hz, 1H), 9.23 (s, 1H), 8.93 (s, 2H), 8.20 (d, J = 8.7 Hz, 1H), 7.99 (s, 1H), 7.65 (t, J = 7.6, 7.6 Hz, 1H), 7.34 (t, J = 9.1, 9.1 Hz, 1H), 7.21-6.99 (m, | DMSO | >98 | Method R5 | | | |
| | | | | | 2H), 3.82 (s, | | | | | | |
| | | | | | 3H), 3.22 (d, J = | | | | | | |
| | | | | | 4.4 Hz, 3H). | | | | | | |
| | | | | | 1H of 2HCl was not | | | | | | |
| | | | | | observed. | | | | | | |
| 1039 | 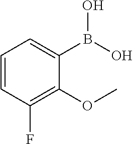 | 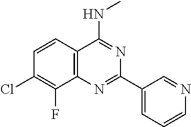 [SMILES: CNc1nc(-c2cccnc2)nc2c(F)c(Cl)ccc12] | 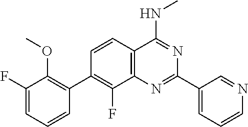 [SMILES: CNc1nc(-c2cccnc2)nc2c(F)c(-c3cccc(F)c3OC)ccc12] | 2HCl | 1H NMR (400 MHz, DMSO) δ 9.69 (d, J = 1.9 Hz, 1H), 9.28 (d, J = 8.2 Hz, 1H), 8.95 (t, J = 5.0, 5.0 Hz, 2H), 8.18 (d, J = 8.5 Hz, 1H), 8.03 (t, J = 6.8, 6.8 Hz, | DMSO | >98 | Method R5 | | | |
| | | | | | 1H), 7.56 (dd, J = | | | | | | |
| | | | | | 8.4, 6.6 Hz, 1H), | | | | | | |
| | | | | | 7.44 (ddd, J = | | | | | | |
| | | | | | 11.8, 6.5, 3.6 Hz, | | | | | | |
| | | | | | 1H), 7.26 (dt, J = | | | | | | |
| | | | | | 5.5, 4.1, 4.1 Hz, | | | | | | |
| | | | | | 2H), 3.73 (s, 3H), | | | | | | |
| | | | | | 3.22 (d, J = 4.3 Hz, | | | | | | |
| | | | | | 3H). 1H of 2HCl | | | | | | |
| | | | | | was not observed. | | | | | | |
| 1040 | 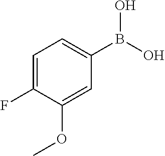 | 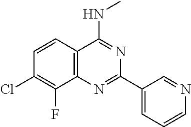 [SMILES: CNc1nc(-c2cccnc2)nc2c(F)c(Cl)ccc12] | 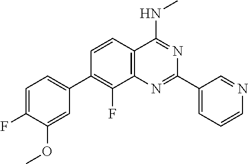 [SMILES: CNc1nc(-c2cccnc2)nc2c(F)c(-c3ccc(F)c(OC)c3)ccc12] | 2HCl | 1H NMR (400 MHz, DMSO) δ 9.69 (d, J = 1.9 Hz, 1H), 9.39 (dt, J = 8.2, 1.7, 1.7 Hz, 1H), 9.19-8.85 (m, 2H), 8.24 (d, J = 8.6 Hz, 1H), 8.15 (dd, J = 8.1, 5.5 Hz, 1H), 7.77 (dd, J = 8.6, 7.1 Hz, | DMSO | >98 | Method R5 | | | |
| | | | | | 1H), 7.48 (dd, | | | | | | |
| | | | | | J = 8.3, 2.0 Hz, | | | | | | |
| | | | | | 1H), 7.40 (dd, J = | | | | | | |
| | | | | | 11.4, 8.4 Hz, 1H), | | | | | | |
| | | | | | 7.29 (ddd, J = | | | | | | |
| | | | | | 8.5, 3.9, 1.7 Hz, | | | | | | |
| | | | | | 1H), 3.94 (s, 3H), | | | | | | |
| | | | | | 3.21 (d, J = 4.3 | | | | | | |
| | | | | | Hz, 3H). H of 2HCl | | | | | | |
| | | | | | was not observed. | | | | | | |
| 1041 | 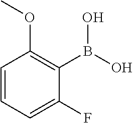 | 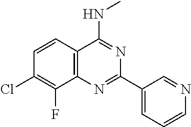 [SMILES: CNc1nc(-c2cccnc2)nc2c(F)c(Cl)ccc12] | 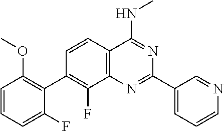 [SMILES: CNc1nc(-c2cccnc2)nc2c(F)c(-c3c(F)cccc3OC)ccc12] | 2HCl | 1H NMR (400 MHz, DMSO) δ 9.69 (d, J = 2.0 Hz, 1H), 9.32 (dt, J = 8.2, 1.8, 1.8 Hz, 1H), 9.05-8.77 (m, 2H), 8.17 (d, J = 9.0 Hz, 1H), 8.08 (dd, J = 8.1, | DMSO | >98 | Method R5 | | | |
| | | | | | 5.4 Hz, 1H), 7.53 | | | | | | |
| | | | | | (ddt, J = 8.4, 6.9, | | | | | | |
| | | | | | 4.4, 4.4 Hz, 2H), | | | | | | |
| | | | | | 7.08 (d, J = 8.5 Hz, | | | | | | |
| | | | | | 1H), 7.05-6.91 | | | | | | |
| | | | | | (m, 1H), 3.84 (s, | | | | | | |
| | | | | | 3H), 3.22 (d, J = | | | | | | |
| | | | | | 4.4 Hz, 3H). 1H of | | | | | | |
| | | | | | 2HCl was not | | | | | | |
| | | | | | observed. | | | | | | |
| 1042 | 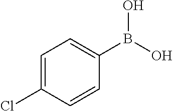 | 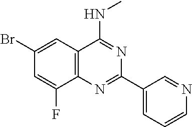 [SMILES: CNc1nc(-c2cccnc2)nc2c(F)cc(Br)cc12] | 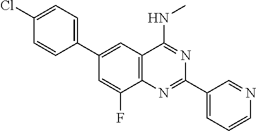 [SMILES: CNc1nc(-c2cccnc2)nc2c(F)cc(-c3ccc(Cl)cc3)cc12] | 2HCl | 1H NMR (400 MHz, DMSO) δ 9.69 (d, J = 2.0 Hz, 1H), 9.22 (d, J = 7.7 Hz, 1H), 9.00 (brd, J = 5.3 Hz, 1H), 8.92 (d, J = 5.2 Hz, 1H), 8.53 (d, J = 1.9 Hz, 1H), 8.14 | DMSO | >98 | Method R3 Temperature at 80° C. | | | |
| | | | | | (dd, J = 12.1, | | | | | | |
| | | | | | 1.7 Hz, 1H), 8.03- | | | | | | |
| | | | | | 7.88 (m, 3H), | | | | | | |
| | | | | | 7.75-7.48 (m, 2H), | | | | | | |
| | | | | | 3.22 (d, J = | | | | | | |
| | | | | | 4.4 Hz, 3H). 1H of | | | | | | |
| | | | | | 2HCl was not | | | | | | |
| | | | | | observed. | | | | | | |
| 1043 | 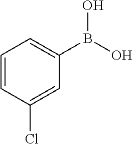 | 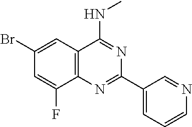 [SMILES: CNc1nc(-c2cccnc2)nc2c(F)cc(Br)cc12] | 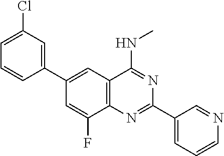 [SMILES: CNc1nc(-c2cccnc2)nc2c(F)cc(-c3cccc(Cl)c3)cc12] | 2HCl | 1H NMR (400 MHz, DMSO) δ 9.69 (d, J = 1.9 Hz, 1H), 9.30 (s, 1H), 9.05 (brs, 1H), 9.00- 8.93 (m, 1H), 8.57-8.47 (m, 1H), 8.19 (dd, J = 12.0, 1.8 Hz, 1H), 8.05 (dd, J = 8.2, 5.4 Hz, 1H), 8.01 | DMSO | >98 | Method R3 Temperature at 80° C. | | | |
| | | | | | (t, J = 1.9, 1.9 Hz, | | | | | | |
| | | | | | 1H), 7.94-7.82 (m, | | | | | | |
| | | | | | 1H), 7.64- | | | | | | |
| | | | | | 7.55 (m, 1H), 7.55- | | | | | | |
| | | | | | 7.48 (m, 1H), | | | | | | |
| | | | | | 3.23 (d, J = 4.3 Hz, | | | | | | |
| | | | | | 3H). 1H of 2HCl | | | | | | |
| | | | | | was not observed. | | | | | | |
| 1044 | 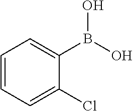 | 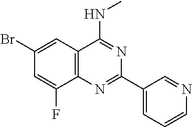 [SMILES: CNc1nc(-c2cccnc2)nc2c(F)cc(Br)cc12] | 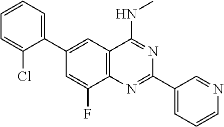 [SMILES: CNc1nc(-c2cccnc2)nc2c(F)cc(-c3ccccc3Cl)cc12] | 2HCl | 1H NMR (400 MHz, DMSO) δ 9.69 (d, J = 2.0 Hz, 1H), 9.17 (s, 1H), 8.97-8.78 (m, 2H), 8.21 (s, 1H), 7.99-7.89 (m, 1H), 7.84 (dd, J = | DMSO | >98 | Method R3 Temperature at 80° C. | | | |
| | | | | | 11.3, 1.7 Hz, 1H), | | | | | | |
| | | | | | 7.70-7.62 (m, | | | | | | |
| | | | | | 1H), 7.62-7.56 (m, | | | | | | |
| | | | | | 1H), 7.56- | | | | | | |
| | | | | | 7.39 (m, 2H), 3.18 | | | | | | |
| | | | | | (d, J = 4.4 Hz, | | | | | | |
| | | | | | 3H). 1H of 2HCl was | | | | | | |
| | | | | | not observed. | | | | | | |
| 1045 | 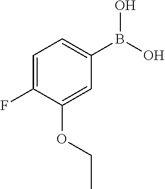 | 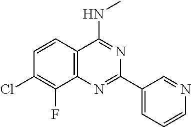 [SMILES: CNc1nc(-c2cccnc2)nc2c(F)c(Cl)ccc12] | 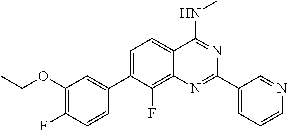 [SMILES: CCOc1cc(-c2ccc3c(NC)nc(-c4cccnc4)nc3c2F)ccc1F] | 2HCl | 1H NMR (400 MHz, DMSO-d6) δ 9.69 (d, J = 2.0 Hz, 1H), 9.32 (dt, J = 8.3, 1.8, 1.8 Hz, 1H), 8.98 (dd, J = 5.5, 1.6 Hz, 2H), 8.21 (d, J = 8.7 Hz, 1H), 8.08 (dd, J = 8.2, 5.5 Hz, 1H), 7.75 (dd, J = 8.6, 7.2 Hz, 1H), | DMSO | >98 | Method R5 | | | |
| | | | | | 7.51-7.24 (m, 3H), | | | | | | |
| | | | | | 4.22 (q, J = | | | | | | |
| | | | | | 6.9 Hz, 2H), 3.21 | | | | | | |
| | | | | | (d, J = 4.4 Hz, | | | | | | |
| | | | | | 3H), 1.39 (t, J = | | | | | | |
| | | | | | 7.0, 7.0 Hz, 3H) | | | | | | |
| | | | | | 1H of 2HCl was | | | | | | |
| | | | | | not observed. | | | | | | |
| 1046 | 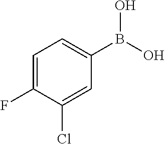 [SMILES: OB(O)c1ccc(F)c(Cl)c1] | 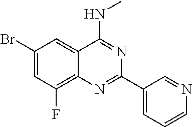 [SMILES: CNc1nc(-c2cccnc2)nc2c(F)cc(Br)cc12] | 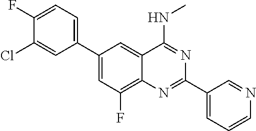 [SMILES: CNc1nc(-c2cccnc2)nc2c(F)cc(-c3ccc(F)c(Cl)c3)cc12] | 2HCl | 1H NMR (400 MHz, DMSO) δ 9.68 (d, J = 2.0 Hz, 1H), 9.23 (d, J = 8.0 Hz, 1H), 8.99 (d, J = 5.2 Hz, 1H), 8.93 (dd, J = 5.4, 1.6 Hz, 1H), 8.52 (d, J = 1.9 Hz, 1H), 8.24-8.12 (m, | DMSO | >98 | Method R3 Temperature at 80° C. | | | |
| | | | | | 2H), 8.06-7.89 (m, | | | | | | |
| | | | | | 2H), 7.62 (t, J = | | | | | | |
| | | | | | 8.9, 8.9 Hz, 1H), | | | | | | |
| | | | | | 3.23 (d, J = 4.4 | | | | | | |
| | | | | | Hz, 3H). 1H of | | | | | | |
| | | | | | 2HCl was not | | | | | | |
| | | | | | observed. | | | | | | |
| 1047 | 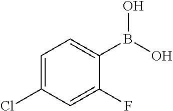 [SMILES: OB(O)c1ccc(Cl)cc1F] | 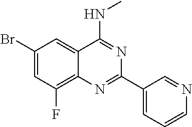 [SMILES: CNc1nc(-c2cccnc2)nc2c(F)cc(Br)cc12] | 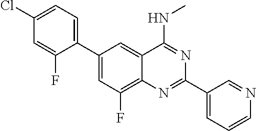 [SMILES: CNc1nc(-c2cccnc2)nc2c(F)cc(-c3ccc(Cl)cc3F)cc12] | 2HCl | 1H NMR (400 MHz, DMSO) δ 9.69 (d, J = 1.9 Hz, 1H), 9.23 (d, J = 8.0 Hz, 1H), 8.98 (d, J = 4.6 Hz, 1H), 8.93 (dd, J = 5.4, 1.6 Hz, 1H), 8.36 (d, J = 1.7 Hz, 1H), | DMSO | >98 | Method R3 Temperature at 80° C. | | | |
| | | | | | 7.99 (dd, J = | | | | | | |
| | | | | | 7.9, 5.5 Hz, 1H), | | | | | | |
| | | | | | 7.93 (dt, J = 11.6, | | | | | | |
| | | | | | 1.6, 1.6 Hz, 1H), | | | | | | |
| | | | | | 7.75 (t, J = 8.5, | | | | | | |
| | | | | | 8.5 Hz, 1H), 7.68 | | | | | | |
| | | | | | (dd, J = 10.7, 2.1 | | | | | | |
| | | | | | Hz, 1H), 7.51 (dd, | | | | | | |
| | | | | | J = 8.3, 2.1 Hz, | | | | | | |
| | | | | | 1H), 3.20 (d, J = | | | | | | |
| | | | | | 4.4 Hz, 3H). 1H of | | | | | | |
| | | | | | 2HCl was not | | | | | | |
| | | | | | observed. | | | | | | |
| 1048 | 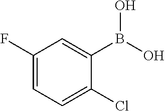 [SMILES: OB(O)c1cc(F)ccc1Cl] | 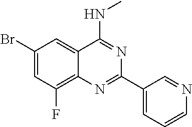 [SMILES: CNc1nc(-c2cccnc2)nc2c(F)cc(Br)cc12] | 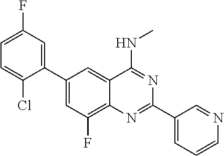 [SMILES: CNc1nc(-c2cccnc2)nc2c(F)cc(-c3cc(F)ccc3Cl)cc12] | 2HCl | 1H NMR (400 MHz, DMSO) δ 9.70 (d, J = 1.9 Hz, 1H), 9.28 (d, J = 8.0 Hz, 1H), 9.04-8.81 (m, 2H), 8.25 (s, 1H), 8.03 (t, J = 6.9, 6.9 Hz, 1H), 7.89 (dd, J = 11.1, 1.7 Hz, 1H), 7.71 (dd, J = | DMSO | >98 | Method R3 Temperature at 80° C. | | | |
| | | | | | 8.9, 5.1 Hz, 1H), | | | | | | |
| | | | | | 7.53 (dd, J = 9.1, | | | | | | |
| | | | | | 3.1 Hz, 1H), 7.40 | | | | | | |
| | | | | | (td, J = 8.5, 8.5, | | | | | | |
| | | | | | 3.1 Hz, 1H), 3.19 | | | | | | |
| | | | | | (d, J = 4.3 Hz, 3H). | | | | | | |
| | | | | | 1H of 2HCl was | | | | | | |
| | | | | | not observed. | | | | | | |
| 1049 | 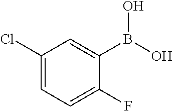 [SMILES: OB(O)c1cc(Cl)ccc1F] | 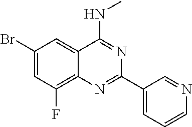 [SMILES: CNc1nc(-c2cccnc2)nc2c(F)cc(Br)cc12] | 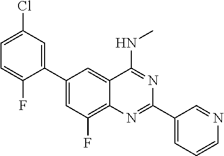 [SMILES: CNc1nc(-c2cccnc2)nc2c(F)cc(-c3cc(Cl)ccc3F)cc12] | HCl | 1H NMR (400 MHz, DMSO) δ 9.67 (dd, J = 2.2, 0.8 Hz, 1H), 8.99 (d, J = 8.0 Hz, 1H), 8.92- 8.68 (m, 2H), 8.42-8.24 (m, 1H), 7.94 (d, J = 11.6 Hz, 1H), 7.81 (dd, J = 6.8, 2.7 | DMSO | >98 | Method R3 Temperature at 80° C. | | | |
| | | | | | Hz, 1H), 7.76 (t, | | | | | | |
| | | | | | J = 6.7, 6.7 Hz, | | | | | | |
| | | | | | 1H), 7.58 (ddd, J = | | | | | | |
| | | | | | 8.9, 4.3, 2.7 Hz, | | | | | | |
| | | | | | 1H), 7.53-7.34 (m, | | | | | | |
| | | | | | 1H), 3.20 (d, J = | | | | | | |
| | | | | | 4.4 Hz, 3H). | | | | | | |
| | | | | | 1H of HCl was not | | | | | | |
| | | | | | observed. | | | | | | |
| 1050 | 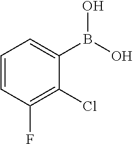 [SMILES: OB(O)c1cccc(F)c1Cl] | 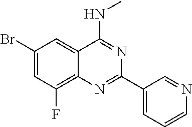 [SMILES: CNc1nc(-c2cccnc2)nc2c(F)cc(Br)cc12] | 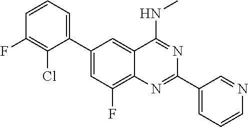 [SMILES: CNc1nc(-c2cccnc2)nc2c(F)cc(-c3cccc(F)c3Cl)cc12] | HCl | 1H NMR (400 MHz, DMSO) δ 9.69 (d, J = 2.0 Hz, 1H), 9.22-8.99 (m, 1H), 8.96-8.79 (m, 2H), 8.28- 8.18 (m, 1H), 8.00- 7.81 (m, 2H), 7.62-7.48 (m, | DMSO | >98 | Method R3 Temperature at 80° C. | | | |
| | | | | | 2H), 7.44 (dd, J = | | | | | | |
| | | | | | 6.5, 2.8 Hz, 1H), | | | | | | |
| | | | | | 3.18 (d, J = 4.4 | | | | | | |
| | | | | | Hz, 3H). 1H of | | | | | | |
| | | | | | 2HCl was not | | | | | | |
| | | | | | observed. | | | | | | |
| 1051 | 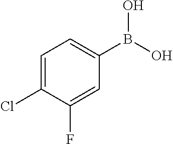 [SMILES: OB(O)c1ccc(Cl)c(F)c1] | 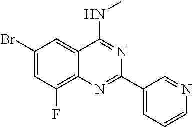 [SMILES: CNc1nc(-c2cccnc2)nc2c(F)cc(Br)cc12] | 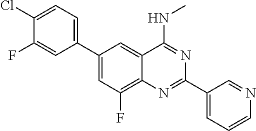 [SMILES: CNc1nc(-c2cccnc2)nc2c(F)cc(-c3ccc(Cl)c(F)c3)cc12] | HCl | 1H NMR (400 MHz, DMSO) δ 9.67 (s, 1H), 9.01 (d, J = 7.8 Hz, 1H), 8.88 (d, J = 5.4 Hz, 1H), 8.82 (d, J = 4.9 Hz, 1H), 8.54 (d, J = 1.9 Hz, 1H), 8.19 (dd, | DMSO | >98 | Method R3 Temperature at 80° C. | | | |
| | | | | | J = 12.1, 1.8 Hz, | | | | | | |
| | | | | | 1H), 8.01 (dd, J = | | | | | | |
| | | | | | 11.1, 1.9 Hz, | | | | | | |
| | | | | | 1H), 7.89-7.70 (m, | | | | | | |
| | | | | | 3H), 3.22 (d, J = | | | | | | |
| | | | | | 4.4 Hz, 3H). 1H | | | | | | |
| | | | | | of HCl was not | | | | | | |
| | | | | | observed. | | | | | | |
| 1052 | 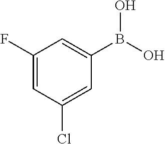 [SMILES: OB(O)c1cc(F)cc(Cl)c1] | 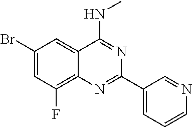 [SMILES: CNc1nc(-c2cccnc2)nc2c(F)cc(Br)cc12] | 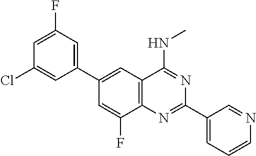 [SMILES: CNc1nc(-c2cccnc2)nc2c(F)cc(-c3cc(F)cc(Cl)c3)cc12] | HCl | 1H NMR (400 MHz, DMSO) δ 9.67 (d, J = 2.0 Hz, 1H), 9.21 (dt, J = 7.9, 1.8, 1.8 Hz, 1H), 9.00 (q, J = 4.4, 4.4, 4.2 Hz, 1H), 8.93 (dd, J = 5.3, 1.6 Hz, 1H), 8.56 (d, J = 1.9 Hz, 1H), 8.23 (dd, | DMSO | >98 | Method R3 Temperature at 80° C. | | | |
| | | | | | J = 12.0, 1.8 Hz, | | | | | | |
| | | | | | 1H), 7.98 (dd, J = | | | | | | |
| | | | | | 8.0, 5.4 Hz, 1H), | | | | | | |
| | | | | | 7.90 (t, J = 1.7, | | | | | | |
| | | | | | 1.7 Hz, 1H), 7.84- | | | | | | |
| | | | | | 7.77 (m, 1H), 7.52 | | | | | | |
| | | | | | (dt, J = 8.6, 2.0, | | | | | | |
| | | | | | 2.0 Hz, 1H), 3.23 | | | | | | |
| | | | | | (d, J = 4.4 Hz, | | | | | | |
| | | | | | 3H). 1H of HCl | | | | | | |
| | | | | | was not observed. | | | | | | |
| 1053 | 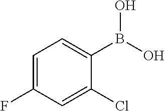 [SMILES: OB(O)c1ccc(F)cc1Cl] | 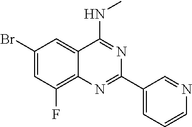 [SMILES: CNc1nc(-c2cccnc2)nc2c(F)cc(Br)cc12] | 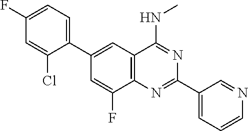 [SMILES: CNc1nc(-c2cccnc2)nc2c(F)cc(-c3ccc(F)cc3Cl)cc12] | HCl | 1H NMR (400 MHz, DMSO) δ 9.69 (d, J = 2.0 Hz, 1H), 9.29 (d, J = 8.1 Hz, 1H), 9.04-8.86 (m, 2H), 8.21 (d, J = 1.8 Hz, 1H), 8.05 (dd, J = 8.1, 5.4 Hz, 1H), | DMSO | >98 | Method R3 Temperature at 80° C. | | | |
| | | | | | 7.84 (dd, J = 11.2, | | | | | | |
| | | | | | 1.6 Hz, 1H), 7.73- | | | | | | |
| | | | | | 7.59 (m, 2H), | | | | | | |
| | | | | | 7.43 (td, J = 8.4, | | | | | | |
| | | | | | 8.4, 2.6 Hz, 1H), | | | | | | |
| | | | | | 3.19 (d, J = 4.4 Hz, | | | | | | |
| | | | | | 3H). 1H of HCl | | | | | | |
| | | | | | was not observed. | | | | | | |
| 1054 | 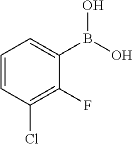 [SMILES: OB(O)c1cccc(Cl)c1F] | 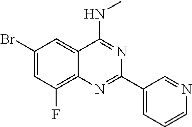 [SMILES: CNc1nc(-c2cccnc2)nc2c(F)cc(Br)cc12] | 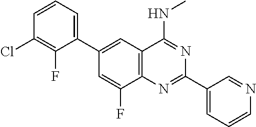 [SMILES: CNc1nc(-c2cccnc2)nc2c(F)cc(-c3cccc(Cl)c3F)cc12] | HCl | 1H NMR (400 MHz, DMSO) δ 9.70 (d, J = 2.0 Hz, 1H), 9.22 (d, J = 8.1 Hz, 1H), 9.03-8.86 (m, 2H), 8.38 (d, J = 1.6 Hz, 1H), 8.07-7.89 (m, 2H), 7.77-7.64 (m, 2H), 7.42 (td, J = | DMSO | >98 | Method R3 Temperature at 80° C. | | | |
| | | | | | 8.0, 7.9, 0.9 Hz, | | | | | | |
| | | | | | 1H), 3.20 (d, J = | | | | | | |
| | | | | | 4.4 Hz, 3H). 1H | | | | | | |
| | | | | | of 2HCl was not | | | | | | |
| | | | | | observed. | | | | | | |

TABLE 37

| | | | | | | | | Meth- | | | |
|---|---|---|---|---|---|---|---|---|---|---|---|
| | | | | | | ¹H | | od of | | Reten- | |
| Num- | Starting | Starting | | Salt | | NMR | Purity | Cou- | | tion | LCMS |
| ber | Material 1 | Material 2 | Product | type | ¹H NMR | Solvent | percent | pling | LCMS | Time | Method |
| 1055 | 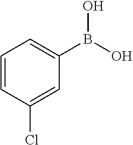 | 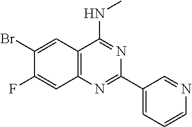 [SMILES: CNc1nc(-c2cccnc2)nc2cc(F)c(Br)cc12] | 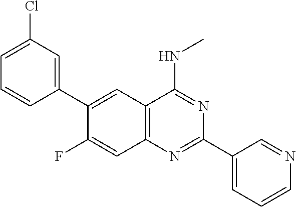 [SMILES: CNc1nc(-c2cccnc2)nc2cc(F)c(-c3cccc(Cl)c3)cc12] | 2HCl | 1H NMR (400 MHz, DMSO) δ 9.66 (d, J = 2.0 Hz, 1H), 9.34 (s, 1H), 9.13 (d, J = 8.1 Hz, 1H), 8.94 (d, J= 5.5 Hz, 1H), 8.64 (d, J = 8.1 Hz, 1H), 7.94 (dd, J = 8.1, 5.2 Hz, 1H), 7.87-7.74 (m, 2H), 7.74-7.65 (m, 1H), 7.65- | DMSO | >98 | R3 at 80 degrees | | | |
| | | | | | 7.52 (m, 2H), 3.24 | | | | | | |
| | | | | | (d, J = 4.4 Hz, 3H). | | | | | | |
| | | | | | 1H of 2HCl was | | | | | | |
| | | | | | not observed. | | | | | | |
| 1056 | 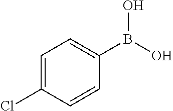 | 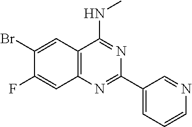 [SMILES: CNc1nc(-c2cccnc2)nc2cc(F)c(Br)cc12] | 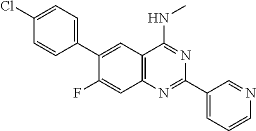 [SMILES: CNc1nc(-c2cccnc2)nc2cc(F)c(-c3ccc(Cl)cc3)cc12] | 2HCl | 1H NMR (400 MHz, DMSO) δ 9.75- 9.60 (m, 1H), 9.60-9.33 (m, 1H), 9.16 (d, J = 8.0 Hz, 1H), 8.96 (dd, J = 5.3, 1.5 Hz, 1H), 8.68 (d, J = 8.0 Hz, 1H), 7.96 (dd, J = 8.0, 5.2 Hz, 1H), 7.85 (d, J = | DMSO | >98 | R3 at 80 degrees | | | |
| | | | | | 11.4 Hz, 1H), 7.79- | | | | | | |
| | | | | | 7.70 (m, 2H), | | | | | | |
| | | | | | 7.70-7.56 (m, | | | | | | |
| | | | | | 2H), 3.24 (d, J = | | | | | | |
| | | | | | 4.4 Hz, 3H). 1H of | | | | | | |
| | | | | | 2HCl were | | | | | | |
| | | | | | not observed. | | | | | | |
| 1057 | 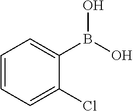 | 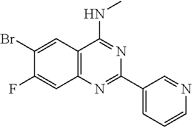 [SMILES: CNc1nc(-c2cccnc2)nc2cc(F)c(Br)cc12] | 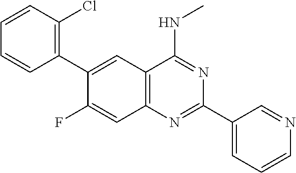 [SMILES: CNc1nc(-c2cccnc2)nc2cc(F)c(-c3ccccc3Cl)cc12] | 2HCl | 1H NMR (400 MHz, DMSO) δ 9.67 (dd, J = 2.0, 0.8 Hz, 1H), 9.13 (brd, J = 7.9 Hz, 2H), 8.95 (dd, J = 5.3, 1.6 Hz, 1H), 8.48 (d, J = 7.4 Hz, 1H), 7.95 (t, J = 6.7, 6.7 Hz, 1H), 7.81 (d, J = 10.6 | DMSO | <98 | R3 at 80 degrees | | | |
| | | | | | Hz, 1H), 7.77- | | | | | | |
| | | | | | 7.61 (m, 1H), | | | | | | |
| | | | | | 7.63-7.48 (m, 3H), | | | | | | |
| | | | | | 3.21 (d, J = | | | | | | |
| | | | | | 4.4 Hz, 3H). | | | | | | |
| | | | | | 1H of HCl was | | | | | | |
| | | | | | not observed. | | | | | | |

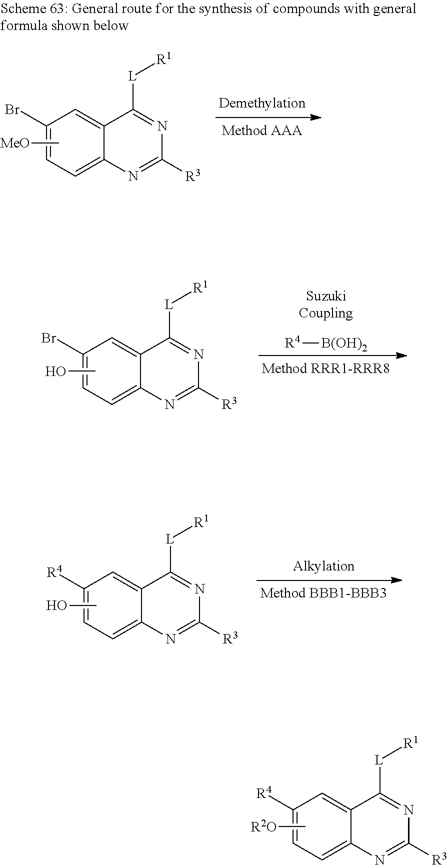

### Method AAA for Demethylation

AAA: BBr₃/CHCl₃, 75° C.

### Method RRR for Coupling Conditions

RRR1: Pd(PPh₃)₂Cl₂/K₂CO₃/Dioxane-H₂O 100° C.

RRR2: Pd(APhos)₂Cl₂/K₃PO₄/Dioxane-H₂O 90° C.

RRR3: Pd(PPh₃)₄/K₂CO₃/DMF-H₂O, 105° C.

RRR4: Pd(APhos)₂Cl₂/CsF/Dioxane, 100° C.

RRR5: Pd(OAc)₂/X-Phos/Cs₂CO₃/Dioxane-H₂O, 90° C.

RRR6: Pd(dppf)Cl₂-CH₂Cl₂/Na₂CO₃ or K₂CO₃/Dioxane-H₂O, reflux

RRR7: Pd(PPh₃)₂Cl₂/K₂CO₃/DME-EtOH-H₂O/microwave, 120° C.

RRR8: Pd(APhos)₂Cl₂/K₃PO₄/Dioxane-H₂O/microwave, 110° C.

### Method BBB for Alkylation

BBB1: DABCO/Cs₂CO₃/DMF, 50° C.

BBB2: Cs₂CO₃/DMF, rt

BBB3: NaH/RX/DMF, 23° C.

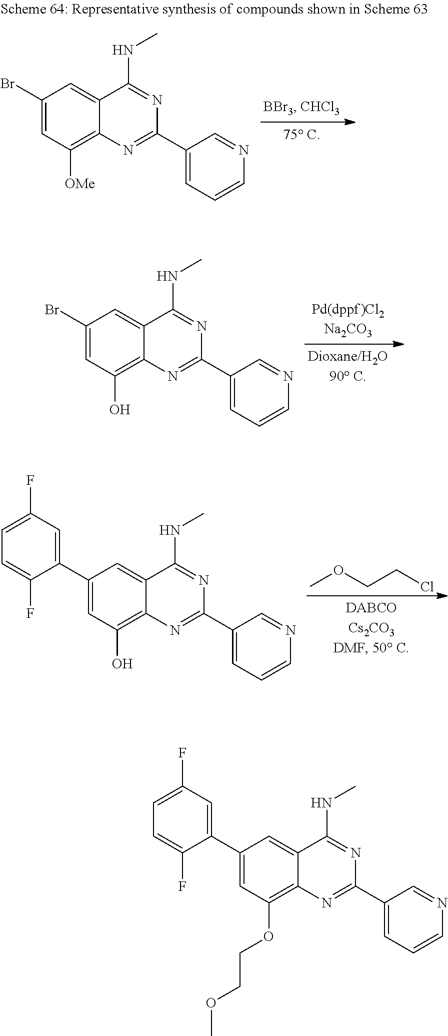

### 6-Bromo-4-(methylamino)-2-(pyridin-3-yl)quinazolin-8-ol (Method AAA)

To a solution of 6-bromo-8-methoxy-N-methyl-2-(pyridin-3-yl)quinazolin-4-amine (2 g, 5.81 mmol) in CHCl₃ (50 mL) was added BBr₃ (14.5 g, 0.058 mol). The reaction mixture was stirred at 75° C. for 24 h. The reaction mixture was cooled and filtered to obtain desired product (1.5 g, 78.4%). MS m/z=331 (M+1) (method AAA) (retention time=1.31 min)

### 6-(2,5-Difluorophenyl)-4-(methylamino)-2-(pyridin-3-yl)quinazolin-8-ol (Method R6)

The desired compound was made using Method RRR6 as described for methyl 3-(4-(methylamino)-2-(pyridin-3-yl)quinazolin-6-yl)benzoate substituting for the appropriate boronic acid in 80% yield. MS m/z=365.0 (M+1) (method BBB) (retention time=1.73 min)

### 6-(2,5-Difluorophenyl)-8-(2-methoxyethoxy)-N-methyl-2-(pyridin-3-yl)quinazolin-4-amine (Method BBB1)

A mixture of 6-bromo-4-(methylamino)-2-(pyridin-3-yl)quinazolin-8-ol (340 mg, 0.93 mmol), 1-chloro-2-ethoxyethane (1.0 g, 9.3 mmol), DABCO (410 mg, 1.86 mmol) and Cs₂CO₃ (3.02 g, 9.3 mmol) in DMF (10 mL) was stirred at 50° C. overnight. After cooling, H₂O (50 mL) was added to the mixture and the resultant precipitate was collected and washed with H₂O to give 320 mg of the desired product in 81.4% yield. LCMS m/z=396.0 (M+1) (method BBB) (retention time=1.714 min) ¹H-NMR (400 MHz, DMSO-d₆): δ 9.57 (s, 1H), 9.23-9.19 (m, 2H), 9.00 (s, 1H), 8.18 (s, 1H), 8.10 (s, 1H), 7.63-7.58 (m, 2H), 7.45 (s, 1H), 7.35 (s, 1H), 4.42 (s, 2H), 3.67-3.64 (m, 2H), 3.19 (s, 3H), 2.52 (s, 2H), 1.18 (t, 3H).

### 6-(2,4-Difluorophenyl)-8-(2-ethoxy)-N-methyl-2-(pyridin-3-yl)quinazolin-4-amine (Method BBB2)

A mixture of 6-bromo-4-(methylamino)-2-(pyridin-3-yl)quinazolin-8-ol (340 mg, 0.93 mmol), iodoethane (1.0 g, 9.3 mmol) and Cs₂CO₃ (3.02 g, 9.3 mmol) in DMF (10 mL) was stirred at rt overnight. Water (50 mL) was added to the mixture and the resultant precipitate was collected and washed with H₂O, MeOH and ether to give the product as the freebase that was converted to the bis-HCl salt using 4M HCl/dioxane to give the desired product as a yellow solid. LCMS m/z=393.2 (M+1) (method CCC) (retention time=2.22 min) ¹H-NMR (300 MHz, DMSO-d₆): δ 9.50 (d, J=18.1 Hz, 2H), 9.22 (d, J=7.6 Hz, 1H), 9.02 (d, J=5.4 Hz, 1H), 8.24-8.02 (m, 2H), 7.78 (dt, J=15.6, 7.8 Hz, 1H), 7.47 (dt, J=11.0, 10.5 Hz, 2H), 7.27 (t, J=8.5 Hz, 1H), 4.32 (q, J=6.7 Hz, 2H), 3.18 (s, 3H), 1.48 (t, J=6.9 Hz, 3H).

The compounds in the following table were prepared in a manner analogous to that described in Scheme 63 and 64

TABLE 38

| | Starting | | | | | | | | | |
|---|---|---|---|---|---|---|---|---|---|---|
| | Material | Starting | | Salt | | ¹H NMR | Purity | Method | | Retention |
| Number | 1 | Material 2 | Product | type | ¹H NMR | Solvent | percent | of Coupling | LCMS | Time |
| 1058 | 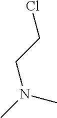 | 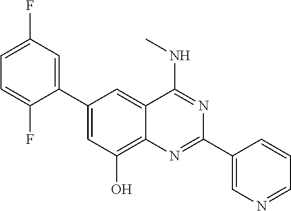 | 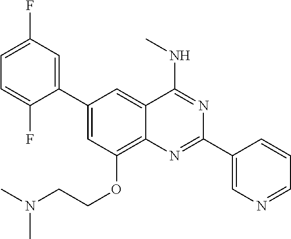 [SMILES: CNc1nc(-c2cccnc2)nc2c(OCCN(C)C)cc(-c3cc(F)ccc3F)cc12] | 2HCl | | DMSO | 97% | Methods RRR6, BBB1 | 436.1 (M + 1) | t = 1.67 min |
| 1059 | 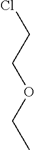 | 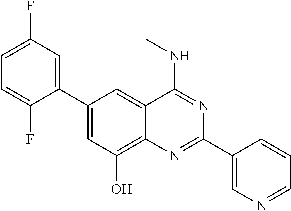 | 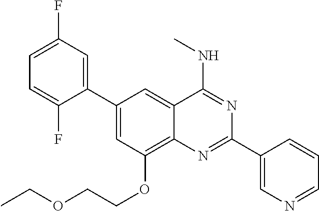 [SMILES: CCOCCOc1cc(-c2cc(F)ccc2F)cc2c(NC)nc(-c3cccnc3)nc12] | 2HCl | 1H-NMR (400 MHz, DMSO-d6): δ 9.57 (s, 1H), 9.23-9.19 (m, 2H), 9.00 (s, 1H), 8.18 (s, 1H), 8.10 (s, 1H), 7.63- 7.58 (m, 2H), 7.45 (s, 1H), 7.35 (s, 1H), 4.42 (s, 2H), 3.67-3.64 (m, 2H), 3.19 (s, 3H), 2.52 (s, 2H), 1.18 (t, 3H). | DMSO | 95 | Methods RRR6, BBB1 | 437.0 (M + 1) | t = 1.714 min |
| 1060 | 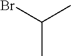 | 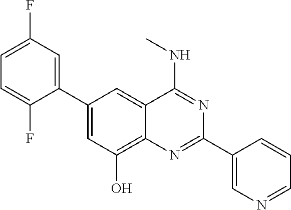 | 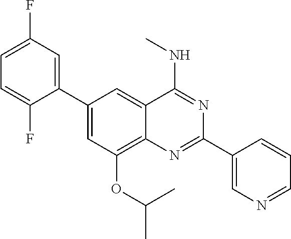 [SMILES: CNc1nc(-c2cccnc2)nc2c(OC(C)C)cc(-c3cc(F)ccc3F)cc12] | | 1H-NMR (400 MHz, DMSO): δ 9.37 (s, 1H), 8.83 (m, 1H), 7.92 (d, J = 1.2 Hz, 1H), 7.83 (m, 1H), 7.64 (s, 1H), 7.35 (s, 1H), 7.20 (s, 1H), 7.15 (s, 1H), 4.95 (m, 1H), 3.30 (s, 3H), 1.44 (d, J = 6.0 Hz, 6H). | DMSO | 95 | Methods RRR6, BBB1 | 406.9 (M + 1) | t = 1.428 min |
| 1061 | 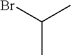 | 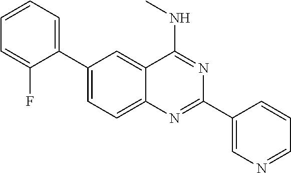 | 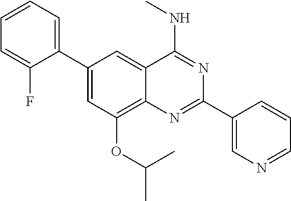 [SMILES: CNc1nc(-c2cccnc2)nc2c(OC(C)C)cc(-c3ccccc3F)cc12] | 2HCl | 1H-NMR (400 MHz, DMSO): δ 9.33 (s, 1H), 8.83 (s, 1H), 8.75 (s, 1H), 7.91 (s, 1H), 7.77 (m, 1H), 7.63 (m, 1H), 7.55 (m, 1H), 7.39 (m, 1H), 7.23 (m, 1H), 4.95 (m, 1H), 3.30 (s, 3H), 1.44 (d, J = 6.4 Hz, 6H). | DMSO | 95 | Methods RRR6, BBB1 | 388.9 (M + 1) | t = 1.402 min |

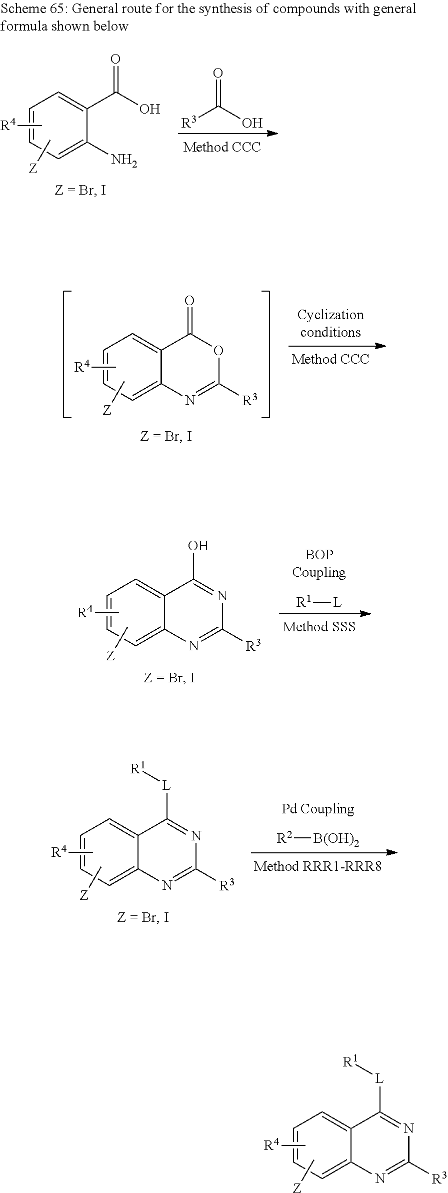

### Method CCC for Amidation/Cyclization

CCC: HATU/DIPEA/DMF, rt then NH₄OH, 54° C.

### Method SSS for Coupling Conditions

SSS: BOP/DBU/MeNH₂/DMF-H₂O, 40° C.

### Method RRR for Coupling Conditions

RRR1: Pd(PPh₃)₂Cl₂/K₂CO₃/Dioxane-H₂O 100° C.

RRR2: Pd(APhos)₂Cl₂/K₃PO₄/Dioxane-H₂O 90° C.

RRR3: Pd(PPh₃)₄/K₂CO₃/DMF-H₂O, 105° C.

RRR4: Pd(APhos)₂Cl₂/CsF/Dioxane, 100° C.

RRR5: Pd(OAc)₂/X-Phos/Cs₂CO₃/Dioxane-H₂O, 90° C.

RRR6: Pd(dppf)Cl₂-CH₂Cl₂/Na₂CO₃ or K₂CO₃/Dioxane-H₂O, reflux

RRR7: Pd(PPh₃)₂Cl₂/K₂CO₃/DME-EtOH-H₂O/microwave, 120° C.

RRR8: Pd(APhos)₂Cl₂/K₃PO₄/Dioxane-H₂O/microwave, 110° C.

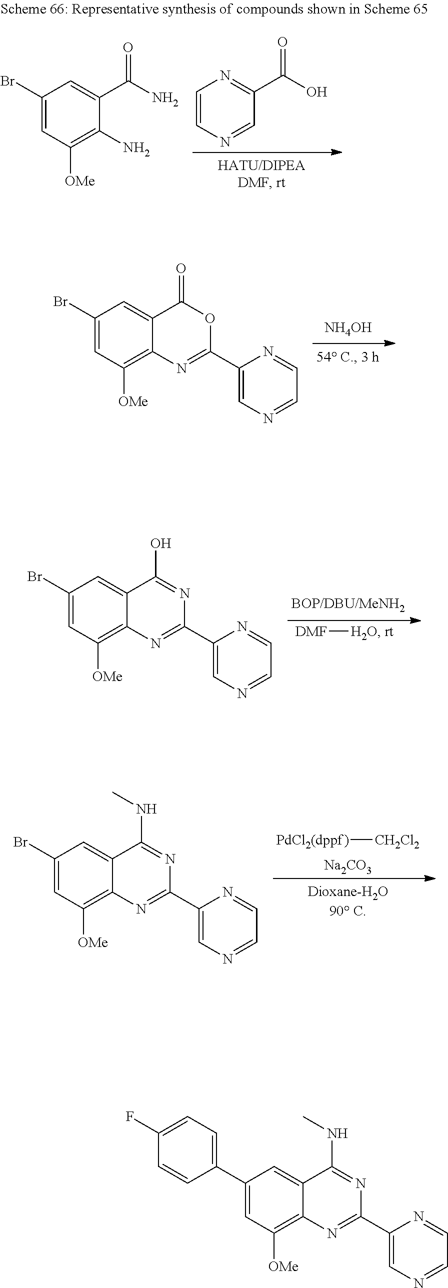

### 6-Bromo-8-methoxy-2-(pyrazin-2-yl)quinazolin-4-ol (Method CCC)

A mixture of pyrazine-2-carboxylic acid (5.12 g, 41.33 mmol) and HATU (39.10 g, 102.9 mmol) in DMF (125 mL) was stirred at rt for 40 min. 2-amino-5-bromo-3-methoxybenzamide (8.4 g, 34.29 mmol) and DIPEA (14.62 g, 113.30 mmol) were added and the mixture was stirred at rt overnight. The mixture was poured into water and filtered to give the product, (6-bromo-8-methoxy-2-(pyrazin-2-yl)-4H-benzo [d][1,3]oxazin-4-one), which was used in the next step without further purification. LCMS m/z=334 (M+1) (method BBB) (retention time=1.28 min)

A mixture of 6-bromo-8-methoxy-2-(pyrazin-2-yl)-4H-benzo[d][1,3]oxazin-4-one (11 g, 33 mmol,) in NH₃—H₂O (400 mL, 28% aqueous solution) was stirred at 54° C. for 3 h. The mixture was concentrated and the pH was adjusted to pH ˜7 with 4N HCl and the resultant precipitate was collected to give the desired product (9.68 g, 85% over 2 steps). LCMS m/z=333 (M+1) (method BBB) (retention time=1.48 min)

### 6-Bromo-8-methoxy-N-methyl-2-phenylquinazolin-4-amine (Method SSS)

A mixture of 6-bromo-8-methoxy-2-(pyrazin-2-yl)quinazolin-4-ol (2.46 g, 7.39 mmol), BOP (6.53 g, 14.77 mmol) and DBU (2.47 g, 16.25 mmol) in DMF (100 ml) was stirred at rt for 1 h. CH₃NH₂—H₂O (120 mL, 40%) was added and stirred at rt for 2 h. and then at 40° C. overnight. After cooling, the mixture was poured into water and the resulting precipitate was filtered to give 6-bromo-8-methoxy-N-methyl-2-(pyrazin-2-yl)quinazolin-4-amine (2.29 g, 89.5%). LCMS m/z=346 (M+1) (method BBB) (retention time=1.44 min)

### 6-(2,4-Difluorophenyl)-8-methoxy-N-methyl-2-(pyrazin-2-yl)quinazolin-4-amine (Method RRR6)

The desired compound was made using Method RRR6 as described for methyl 3-(4-(methylamino)-2-(pyridin-3-yl)quinazolin-6-yl)benzoate substituting for the appropriate boronic acid. LCMS m/z=380.0 (M+1) (method BBB) (retention time=1.55 mm) ¹H-NMR (400 MHz, DMSO-d₆): δ 9.85 (s, 1H), 9.72 (s, 1H), 8.94 (s, 2H), 8.25 (s, 1H), 7.69 (s, 1H), 7.58-7.56 (m, 2H), 7.43-7.38 (m, 1H), 4.11 (s, 3H), 3.27 (s, 3H).

The compounds in the following table were prepared in a manner analogous to that described in Schemes 66 and 67, replacing 2,4-difluorophenyllboronic acid with the appropriate boronic acid/ester.

TABLE 39

| | | | | | | | Puri- | Method | | |
|---|---|---|---|---|---|---|---|---|---|---|
| | | | | | | ¹H | ty | of | | Reten- |
| Num- | Starting | Starting | | Salt | | NMR | per- | Cou- | | tion |
| ber | Material 1 | Material 2 | Product | type | ¹H NMR | Solvent | cent | pling | LCMS | Time |
| 1062 | 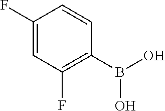 [SMILES: OB(O)c1ccc(F)cc1F] | 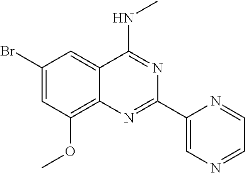 [SMILES: CNc1nc(-c2cnccn2)nc2c(OC)cc(Br)cc12] | 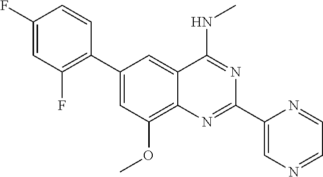 [SMILES: CNc1nc(-c2cnccn2)nc2c(OC)cc(-c3ccc(F)cc3F)cc12] | 2HCl | 1H-NMR (400 MHz, DMSO-d6): δ 10.16 (s, 1H), 9.74 (d, J = 1.6 Hz, 1H), 8.99 (d, J = 2.8 Hz, 1H), 8.96- 8.95 (m, 1H), 8.23 (s, 1H), 7.83- 7.77 (m, 1H), 7.70 (s, 1H), 7.53- 7.47 (m, 1H), 7.35-7.30 (m, 1H), | DMSO | 100 | Method RRR6 | 380.0 (M + 1) | t = 1.554 min |
| | | | | | 4.13 (s, 3H), 3.31 | | | | | |
| | | | | | (d, J = 4.4 Hz, | | | | | |
| | | | | | 3H). | | | | | |
| 1063 | 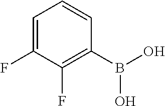 [SMILES: OB(O)c1cccc(F)c1F] | 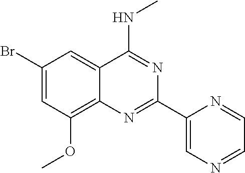 [SMILES: CNc1nc(-c2cnccn2)nc2c(OC)cc(Br)cc12] | 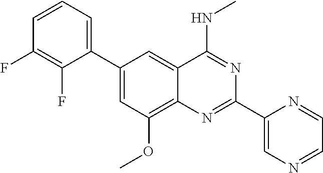 [SMILES: CNc1nc(-c2cnccn2)nc2c(OC)cc(-c3cccc(F)c3F)cc12] | HCl | 1H-NMR (400 MHz, DMSO-d6): δ 9.85 (s, 1H), 9.72 (s, 1H), 8.94 (s, 2H), 8.25 (s, 1H), 7.69 (s, 1H), 7.58- 7.56 (m, 2H), 7.43-7.38 (m, 1H), 4.11 (s, 3H), 3.27(s, 3H). | DMSO | 95 | Method RRR6 | 380.0 (M + 1) | t = 1.547 min |
| 1064 | 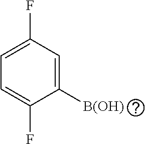 | 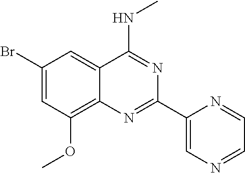 [SMILES: CNc1nc(-c2cnccn2)nc2c(OC)cc(Br)cc12] | 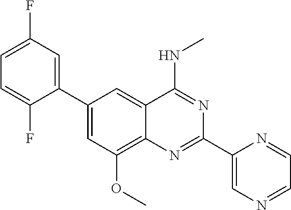 [SMILES: CNc1nc(-c2cnccn2)nc2c(OC)cc(-c3cc(F)ccc3F)cc12] | HCl | 1H-NMR (400 MHz, DMSO-d6): δ 9.63 (s, 1H), 8.81 (s, 1H), 8.74 (d, J = 2.4 Hz, 1H), 8.51 (d, J = 4.4 Hz, 1H), 8.03 (s, 1H), 7.66 (dd, J = 6.4, 2.8 Hz, 1H), 7.47- 7.36 (m, 3H), 4.02(s, 3H), 3.14 (d, J = 4.4 Hz, 3H). | DMSO | 95 | Method RRR6 | 380.0 (M + 1) | t = 1.611 min |

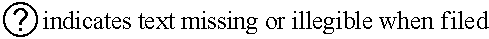

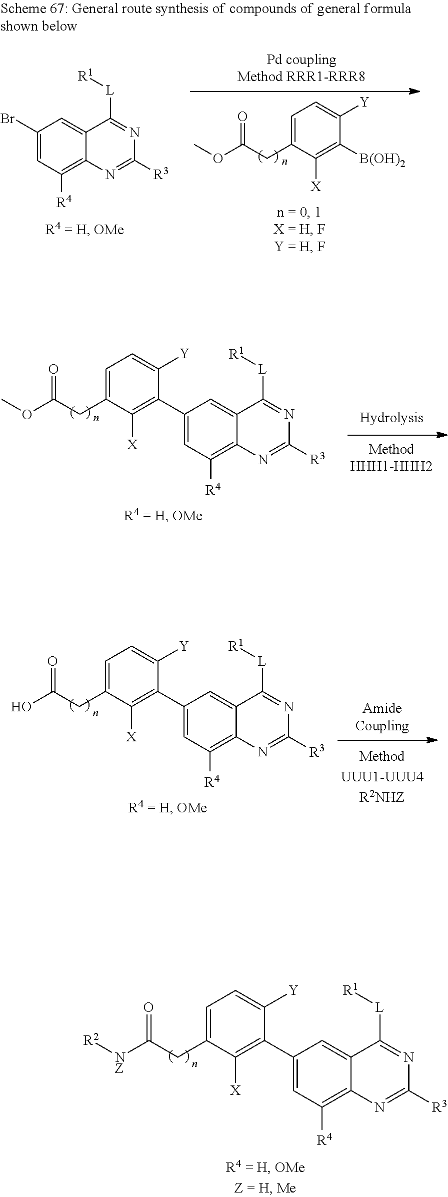

### Method RRR for Pd Coupling Conditions

RRR1: Pd(PPh₃)₂Cl₂/K₂CO₃/Dioxane-H₂O 100° C.

RRR2: Pd(APhos)₂Cl₂/K₃PO₄/Dioxane-H₂O 90° C.

RRR3: Pd(PPh₃)₄/K₂CO₃/DMF-H₂O, 105° C.

RRR4: Pd(APhos)₂Cl₂/CsF/Dioxane, 100° C.

RRR5: Pd(OAc)₂/X-Phos/Cs₂CO₃/Dioxane-H₂O, 90° C.

RRR6: Pd(dppf)Cl₂-CH₂Cl₂/Na₂CO₃ or K₂CO₃/Dioxane-H₂O, reflux

RRR7: Pd(PPh₃)₂Cl₂/K₂CO₃/DME-EtOH-H₂O/microwave, 120° C.

RRR8: Pd(APhos)₂Cl₂/K₃PO₄/Dioxane-H₂O/microwave, 110° C.

### Method HHH for Hydrolysis

HHH1: NaOH, MeOH—H₂O, 50° C.

HHH2: conc. HCl, reflux

### Method UUU for Amide Coupling

UUU1: EDCI/HOBt/NMP, 60° C.

UUU2: HATU/DIPEA/DMF, 23° C.

UUU3: SOCl₂, reflux then NaH/pyridine/DMAP, 23° C.

UUU4: HATU/Pyridine, 23° C.

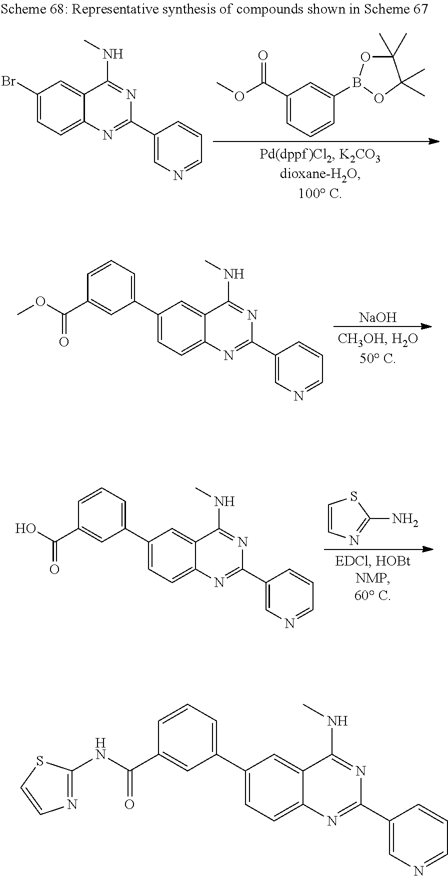

### Methyl 3-(4-(methylamino)-2-(pyridin-3-yl)quinazolin-6-yl)benzoate (Method RRR6)

A mixture of 6-bromo-N-methyl-2-(pyridin-3-yl)quinazolin-4-amine (5.30 g, 16.82 mmol), methyl 3-(4,4,5,5-tetramethyl-1,3,2-dioxaborolan-2-yl)benzoate (5.30 g, 20.22 mmol), Pd(dppf)Cl₂ (650 mg, 0.89 mmol) and K₂CO₃ (7.00 g, 50.64 mmol) was added to dioxane (350 mL) and water (25 mL) and heated at reflux overnight under a N₂ atmosphere. The volatiles were removed in-vacuo and the residue was purified by chromatography (silica gel, isocratic gradient of petroleum ether and ethyl acetate 1:1, with 3% TEA) to give methyl 3-(4-(methylamino)-2-(pyridin-3-yl)quinazolin-6-yl)benzoate (4.20 g, 67.4%). LCMS m/z=371 (M+1) (method BBB) (retention time=1.62 min)

### 3-(4-(Methylamino)-2-(pyridin-3-yl)quinazolin-6-yl)benzoic acid (Method HHH1)

To a solution of methyl 3-(4-(methylamino)-2-(pyridin-3-yl)quinazolin-6-yl)benzoate (4.20 g, 11.34 mmol) in methanol (200 mL) and water (20 mL) was added NaOH (1.40 g, 35.0 mmol). The mixture was stirred at 50° C. overnight. After cooling, the volatiles were removed in-vacuo and the residue was adjusted to pH 2 with 4N HCl. After filtration, 3-(4-(methylamino)-2-(pyridin-3-yl)quinazolin-6-yl)benzoic acid (3.26 g, 80.7%) was obtained. LCMS m/z=357 (M+1) (method BBB) (Retention time=1.25 min)

### 3-(4-(Methylamino)-2-(pyridin-3-yl)quinazolin-6-yl)-N-(thiazol-2-yl)benzamide (Method UUU1)

A solution of 3-(4-(methylamino)-2-(pyridin-3-yl)quinazolin-6-yl)benzoic acid (700 mg, 1.96 mmol), EDCI (452 mg, 2.36 mmol) and HOBt (320 mg, 2.37 mmol) in NMP (15 ml) was stirred at rt for 1 h and thiazol-2-amine (217 mg, 2.17 mmol) was added. The mixture was stirred at 60° C. overnight. After cooling, 100 mL of water was added to the mixture and a precipitate formed. The solid was collected and purified on reverse phase chromatography to give 3-(4-(methylamino)-2-(pyridin-3-yl)quinazolin-6-yl)-N-(thiazol-2-yl)benzamide (133.9 mg, 15.6%). LCMS m/z=439 (M+1) (method BBB) (Retention time=1.64 min) ¹H NMR (400 MHz, DMSO) δ 12.84 (s, 1H), 9.67 (s, 1H), 8.80 (d, J=8.0 Hz, 1H), 8.70 (s, 3H), 8.62 (s, 1H), 8.33 (d, J=8.5 Hz, 1H), 8.12 (d, J=7.6 Hz, 2H), 7.92 (d, J=8.8 Hz, 1H), 7.72 (t, J=7.6 Hz, 1H), 7.59 (d, J=3.4 Hz, 1H), 7.56 (dd, J=7.8, 5.0 Hz, 1H), 7.30 (d, J=2.8 Hz, 1H), 3.21 (d, J=4.2 Hz, 3H).

The compounds in the following table were prepared in a manner analogous to that described in Schemes 67 and 68, replacing thiazol-2-amine with the appropriate amine.

TABLE 40

| | | | | | | | Puri- | | | |
|---|---|---|---|---|---|---|---|---|---|---|
| | Starting | | | | | ¹H | ty | Method | | Reten- |
| Num- | Material | Starting | | Salt | | NMR | per- | of | | tion |
| ber | 1 | Material 2 | Product | Type | ¹H NMR | Solvent | cent | Coupling | LCMS | Time |
| 1065 | 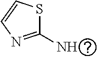 | 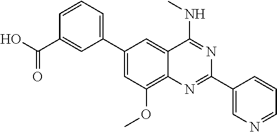 | 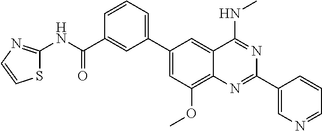 [SMILES: CNc1nc(-c2cccnc2)nc2c(OC)cc(-c3cccc(C(=O)Nc4nccs4)c3)cc12] | | 1H-NMR (400 MHz, DMSO-d6): δ 9.62 (d, J = 1.2 Hz, 1H), 9.01 (d, J = 8.0 Hz, 1H), 8.84 (d, J = 4.0 Hz, 1H), 8.56 (s, 1H), 8.17-8.05 (m, 3H), 7.89- 7.55 (m, 4H), 7.33 (d, J = 3.6 Hz, 1H), 4.13 (d, J = 6.6 Hz, | DMSO | 95 | Methods HHH1, UUU2 | 469.0 (M + 1) | t = 1.587 min |
| | | | | | 3H), 3.22 (d, J = | | | | | |
| | | | | | 4.4 Hz, 3H). | | | | | |
| 1066 | 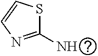 | 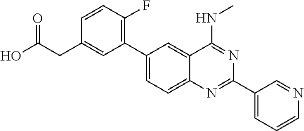 | 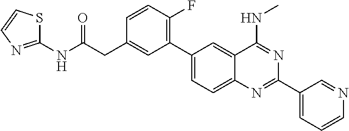 [SMILES: CNc1nc(-c2cccnc2)nc2ccc(-c3cc(CC(=O)Nc4nccs4)ccc3F)cc12] | | 1H-NMR (400 MHz, DMSO-d6): δ 12.40 (s, 1H), 9.65 (d, J = 2.0 Hz, 1H), 8.81 (dd, J = 2.0, 4.8 Hz, 1H), 8.71- 8.65 (m, 2H), 8.45 (s, 1H), 7.95 (d, J = 7.6 Hz, 1H), | DMSO | 95 | Methods HHH1, UUU1 | 471.0 (M + 1) | t = 1.748 min |
| | | | | | 7.88 (d, J = 8.8 Hz, | | | | | |
| | | | | | 1H), 7.62-7.57 (m, | | | | | |
| | | | | | 2H), 7.48 (d, J = | | | | | |
| | | | | | 3.6 Hz, 1H), 7.43- | | | | | |
| | | | | | 7.38 (m, 2H), | | | | | |
| | | | | | 7.21 (d, J = 3.6 | | | | | |
| | | | | | Hz, 1H), 3.88 (s, | | | | | |
| | | | | | 2H), 3.17 (s, 3H). | | | | | |
| 1067 | 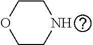 | 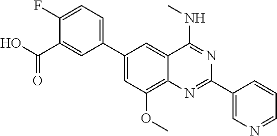 | 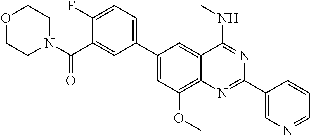 [SMILES: CNc1nc(-c2cccnc2)nc2c(OC)cc(-c3ccc(F)c(C(=O)N4CCOCC4)c3)cc12] | | 1H-NMR (400 MHz, DMSO-d6): δ 9.63 (d, J = 1.6 Hz, 1H), 8.76 (td, J = 3.6, 2.0 Hz, 1H), 8.67 (dd, J = 4.4, 1.6 Hz, 1H), 8.52-8.51 (m, 1H), 8.14 (d, J = 0.8 Hz, 1H), 8.06- | DMSO | 95 | Methods HHH1, UUU1 | 474.0 (M + 1) | t = 1.490 min |
| | | | | | 8.02 (m, 1H), | | | | | |
| | | | | | 7.95 (dd, J = 6.4, | | | | | |
| | | | | | 2.4 Hz, 1H), 7.57- | | | | | |
| | | | | | 7.47 (m, 3H), 4.08 | | | | | |
| | | | | | (s, 3H), 3.70 (s, | | | | | |
| | | | | | 4H), 3.58 (s, 2H), | | | | | |
| | | | | | 3.32 (s, 2H), 3.18 | | | | | |
| | | | | | (d, J = 4.0 Hz, 3H) | | | | | |
| 1068 | 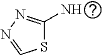 | 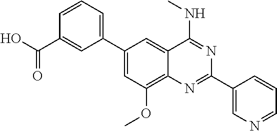 | 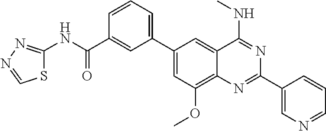 [SMILES: CNc1nc(-c2cccnc2)nc2c(OC)cc(-c3cccc(C(=O)Nc4nncs4)c3)cc12] | | 1H-NMR (400 MHz, DMSO-d6): δ 9.63 (s, 1H), 9.28 (s, 1H), 8.97 (d, J = 8.0 Hz, 1H), 8.82 (d, J = 4.2 Hz, 2H), 8.59 (s, 1H), 8.30-8.10 (m, 3H), 7.77- 7.65 (m, 3H), 4.14 | DMSO | 95 | Methods HHH1, UUU1 | 470.1 (M + 1) | t = 1.447 min |
| | | | | | (s, 3H), 3.22 (d, J = | | | | | |
| | | | | | 4.4 Hz, 3H). | | | | | |
| 1069 | 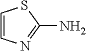 | 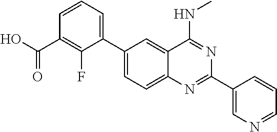 | 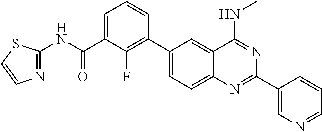 [SMILES: CNc1nc(-c2cccnc2)nc2ccc(-c3cccc(C(=O)Nc4nccs4)c3F)cc12] | | 1H-NMR (400 MHz, DMSO-d6): δ 12.69 (s, 1H), 9.65 (s, 1H), 8.80 (d, J = 7.6 Hz, 1H), 8.70 (d, J = 4.4 Hz, 1H), 8.64 (d, J = 4.8 Hz, 1H), 8.50 (s, 1H), 8.05 (d, J = | DMSO | 95 | Methods HHH1, UUU1 | 456.9 (M + 1) | t = 1.588 min |
| | | | | | 8.4 Hz, 1H), 7.91 (d, | | | | | |
| | | | | | J = 8.8 Hz, 1H), | | | | | |
| | | | | | 7.86 (t, J = 7.6 Hz, | | | | | |
| | | | | | 1H), 7.77 (t, J = | | | | | |
| | | | | | 6.8 Hz, 1H), | | | | | |
| | | | | | 7.56-7.54 (m, 2H), | | | | | |
| | | | | | 7.50 (t, J = 7.6 | | | | | |
| | | | | | Hz, 1H), 7.32 (d, J = | | | | | |
| | | | | | 3.2 Hz, 1H), 3.17 | | | | | |
| | | | | | (d, J = 3.6 Hz, 3H). | | | | | |
| 1070 | 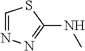 | 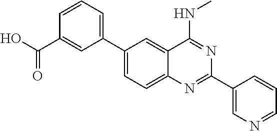 | 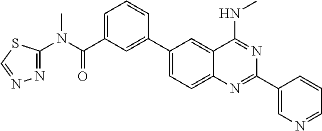 [SMILES: CNc1nc(-c2cccnc2)nc2ccc(-c3cccc(C(=O)N(C)c4nncs4)c3)cc12] | | 1H-NMR (400 MHz, DMSO-d6): δ 9.64 (d, J = 1.6 Hz, 1H), 9.34 (s, 1H), 8.80 (td, J = 8.0, 1.8 Hz, 1H), 8.75 (s, 1H), 8.71 (d, J = 3.6 Hz, 1H), 8.68 (d, J = 2.0 Hz, 1H), 8.26 | DMSO | 99 | Methods, HHH1, UUU2 | 454.1 (M + 1) | t = 1.560 min |
| | | | | | (dd, J = 8.8, 1.6 Hz, | | | | | |
| | | | | | 1H), 8.16 (s, | | | | | |
| | | | | | 1H), 8.11-8.09 (m, | | | | | |
| | | | | | 1H), 7.90 (d, J = | | | | | |
| | | | | | 8.8 Hz, 1H), 7.74 (s, | | | | | |
| | | | | | 1H), 7.73-7.71 (m, | | | | | |
| | | | | | 1H), 7.58 (dd, J = | | | | | |
| | | | | | 8.0, 4.8 Hz, 1H), | | | | | |
| | | | | | 3.71 (s, 3H), 3.21 | | | | | |
| | | | | | (d, J = 4.4 Hz, 3H). | | | | | |
| 1071 | 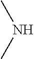 | 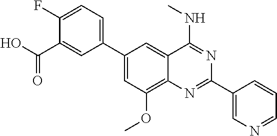 | 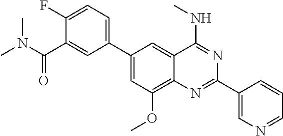 [SMILES: CNc1nc(-c2cccnc2)nc2c(OC)cc(-c3ccc(F)c(C(=O)N(C)C)c3)cc12] | | 1H-NMR (400 MHz, DMSO-d6): δ 9.63 (s, 1H), 8.76 (d, J = 7.2 Hz, 1H), 8.65 (d, J = 4.2 Hz, 1H), 8.42 (t, J = 4.4 Hz, 1H), 8.12- 8.00 (m, 3H), 7.56- 7.43 (m, 3H), | DMSO | 95 | Methods HHH1, UUU1 | 432.0 (M + 1) | t = 1.517 min |
| | | | | | 4.07(s, 3H), 3.17 (d, | | | | | |
| | | | | | J = 4.0 Hz, 3H), | | | | | |
| | | | | | 2.83 (d, J = 4.4 Hz, | | | | | |
| | | | | | 3H). | | | | | |
| 1072 | 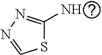 | 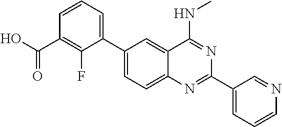 | 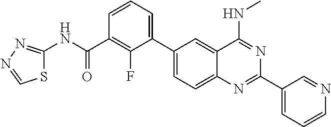 | 3HCl | 1H-NMR (400 MHz, DMSO-d6): δ 9.67 (s, 1H), 9.29 (s, 1H), 9.01-8.95 (m, 2H), 8.77 (s, 1H), 8.24 (s, 2H), 8.01- 7.80 (m, 3H), 7.56 (t, J = 7.6 Hz, 1H), | DMSO | 95 | Methods HHH1, UUU1 | 432.0 (M + 1) | t = 1.517 min |
| | | | | | 3.29 (d, J = | | | | | |
| | | | | | 4.19 Hz, 3H) | | | | | |
| 1073 | 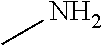 | 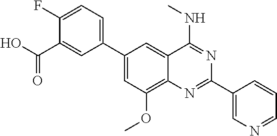 | 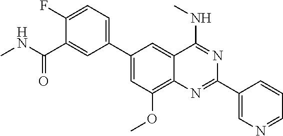 [SMILES: CNC(=O)c1cc(-c2cc(OC)c3nc(-c4cccnc4)nc(NC)c3c2)ccc1F] | | 1H-NMR (400 MHz, DMSO-d6): δ 9.63 (s, 1H), 8.76 (d, J = 7.2 Hz, 1H), 8.65 (d, J = 4.2 Hz, 1H), 8.42 (t, J = 4.4 Hz, 1H), 8.12- 8.00 (m, 3H), 7.56- 7.43 (m, 3H), 4.07 | DMSO | 95 | Methods HHH1, UUU1 | 418.1 (M + 1) | t = 1.482 min |
| | | | | | (s, 3H), 3.17 (d, | | | | | |
| | | | | | J = 4.0 Hz, 3H), | | | | | |
| | | | | | 2.83 (d, J = 4.4 Hz, | | | | | |
| | | | | | 3H). | | | | | |

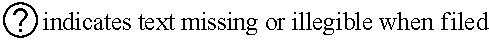

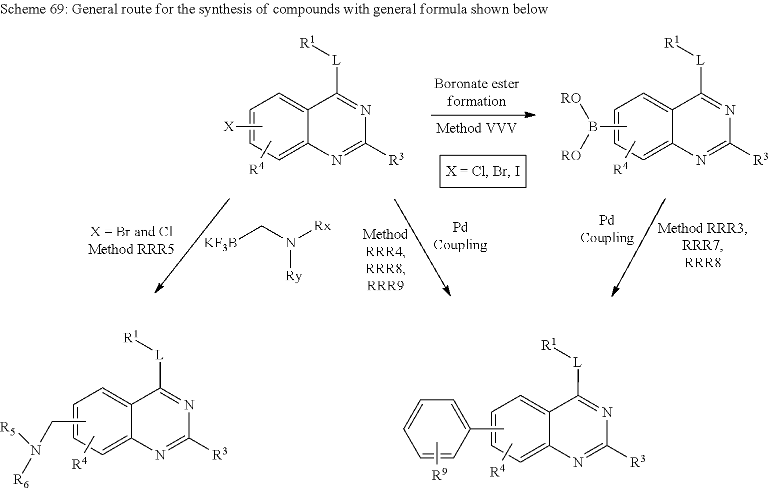

### Method VVV for Boronate Ester Formation

VVV: Pd(dppf)Cl₂/KOAc/Dioxane, 110° C.

### Method RRR for Pd Coupling Conditions

RRR1: Pd(PPh₃)₂Cl₂/K₂CO₃/Dioxane-H₂O 100° C.

RRR2: Pd(APhos)₂Cl₂/K₃PO₄/Dioxane-H₂O 90° C.

RRR3: Pd(PPh₃)₄/K₂CO₃/DMF-H₂O, 105° C.

RRR4: Pd(APhos)₂Cl₂/CsF/Dioxane, 100° C.

RRR5: Pd(OAc)₂/X-Phos/Cs₂CO₃/Dioxane-H₂O, 90° C.

RRR6: Pd(dppf)Cl₂-CH₂Cl₂/Na₂CO₃ or K₂CO₃/Dioxane-H₂O, reflux

RRR7: Pd(PPh₃)₂Cl₂/K₂CO₃/DME-EtOH-H₂O/microwave, 120° C.

RRR8: Pd(APhos)₂Cl₂/K₃PO₄/Dioxane-H₂O/microwave, 110° C.

RRR9: Pd(PPh₃)₄/Stannane/Dioxane/microwave, 125° C.

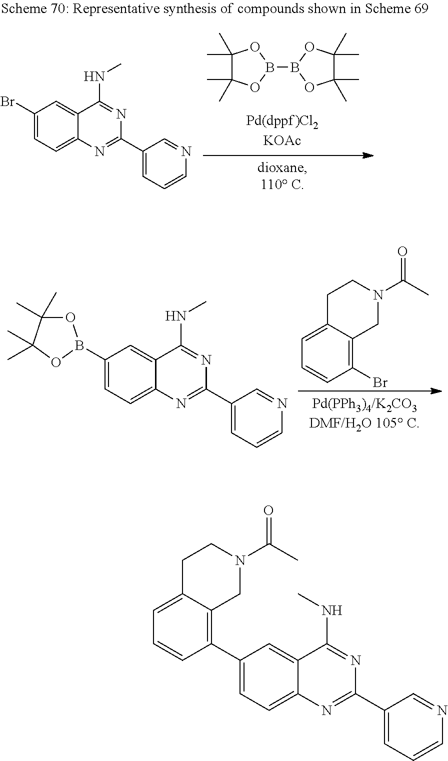

### N-Methyl-2-(pyridin-3-yl)-6-(4, 4,5,5-tetramethyl-1,3,2-dioxaborolan-2-yl) quinazolin-4-amine (Method VVV)

A flask was charged with 6-bromo-N-methyl-2-(pyridin-3-yl)quinazolin-4-amine (5.00 g, 15.86 mmol), bis(pinacolato)diboron (8.05 g, 31.72 mmol, 2.0 equiv), Pd(dppf)Cl₂ (1.29 g, 1.58 mmol, 10 mol %) and potassium acetate (6.22 g, 63.45 mmol, 4.0 equiv). The mixture was suspended in dioxane (350 mL) and the reaction was heated under an argon atmosphere at 110° C. overnight. After cooling, the volatiles were removed in-vacuo. The residue was purified using chromatography (silica gel, gradient of petroleum ether: ethyl acetate from 100:1 to 10:1). N-methyl-2-(pyridin-3-yl)-6-(4,4,5, 5-tetramethyl-1,3,2-dioxaborolan-2-yl) quinazolin-4-amine (3.33 g, 58% yield) was obtained as a light yellow solid. LCMS m/z=363.1 (M+1) (Method BBB) (retention time=1.83 min) ¹H NMR (400 MHz, CDCl₃) δ 9.82 (s, 1H), 8.85 (d, J=8.0 Hz, 1H), 8.74 (s, 1H), 8.21 (s, 1H), 8.12 (d, J=8.8 Hz, 1H), 7.88 (d, J=8.4 Hz, 1H), 7.43 (s, 1H), 6.06 (s, 1H), 3.32 (d, J=4.8 Hz, 3H), 1.38 (s, 12H).

### 1-(8-(4-(Methylamino)-2-(pyridin-3-yl)quinazolin-6-yl)-3,4-dihydroisoquinolin-2(1H)-yl)ethanone (Method RRR3)

A 25 mL reaction flask was charged with N-methyl-2-(pyridin-3-yl)-6-(4,4,5,5-tetramethyl-1,3,2-dioxaborolan-2-yl)quinazolin-4-amine (100 mg, 0.276 mmol), 1-(8-bromo-3,4-dihydroisoquinolin-2(1H)-yl)ethanone (70.2 mg, 0.276 mmol), Pd(PPh₃)₄ (12.7 mg, 0.011 mmol, 4 mol %) and K₂CO₃ (114.5 mg, 0.828 mmol). The mixture was suspended in DMF/H₂O (20:1, 6 mL), and the reaction was heated at 105° C. for 4 h. After cooling, the reaction was diluted with water (30 mL) and the resultant precipitate was collected by filtration. The crude product was purified on prep-HPLC (isocratic gradient 50% MeCN:H₂O, retention time=15 min) to give the desired product as a yellow solid (50 mg, 44%). LCMS m/z=410.2 (M+1) (Method BBB) (retention time=1.72 min) ¹H NMR (300 MHz, DMSO-d₆): δ 9.67 (s, 1H), 8.81-8.68 (m, 2H), 8.29-8.21 (m, 2H), 7.89-7.75 (m, 2H), 7.56-7.51 (m, 1H), 7.35-7.22 (m, 3H), 4.55 (s, 2H), 3.72-3.68 (m, 2H), 3.20-3.18 (m, 3H), 3.05-2.96 (m, 2H), 2.02 (brs, 3H).

### 4-Ethyl-7-(4-(methylamino)-2-(pyridin-3-yl)quinazolin-6-yl)-2H-benzo[b][1,4]thiazin-3(4H)-one (Method RRR7)

To a 10 mL microwave vial were added N-methyl-2-(pyridin-3-yl)-6-(4,4,5,5-tetramethyl-1,3,2-dioxaborolan-2-yl)quinazolin-4-amine (0.250 g, 0.690 mmol), 7-bromo-4-ethyl-2H-benzo[b][1,4]thiazin-3(4H)-one (0.225 g, 0.828 mmol), trans-dichlorobis(triphenylphosphine)palladium (II) (Pd(PPh₃)₂Cl₂) (0.024 g, 0.035 mmol) and potassium carbonate (0.477 g, 3.45 mmol) in DME (1 ml)/water (0.429 ml)/ethanol (0.286 ml) to give a brown suspension. The reaction mixture was then heated to 120° C. for 10 min using microwave irradiation. LC-MS analysis of the crude mixture showed the reaction was complete. The reaction mixture was diluted with water and the resultant precipitate was collected by filtration. The crude solid was purified via ISCO (silica gel, isocratic gradient of 96:4 CH₂Cl₂/MeOH, 24 gm column). The fractions were concentrated and dried under vacuum to give the desired product as a pale brown powder in 37.8% yield. LCMS m/z=428.3 (M+1) (Method CCC) (retention time=2.19 min) ¹H NMR (300 MHz, DMSO-d₆): δ 9.63 (s, 1H), 8.76 (d, J=8.0 Hz, 1H), 8.67 (d, J=4.6 Hz, 1H), 8.65-8.56 (m, 2H), 8.15 (d, J=8.7 Hz, 1H), 7.92 (d, J=2.0 Hz, 1H), 7.80 (dd, J=15.3, 8.7 Hz, 2H), 7.53 (dd, J=7.9, 4.7 Hz, 1H), 7.45 (d, J=8.7 Hz, 1H), 4.03 (q, J=6.8 Hz, 2H), 3.57 (s, 2H), 3.18 (d, J=4.2 Hz, 3H), 1.16 (t, J=6.9 Hz, 3H).

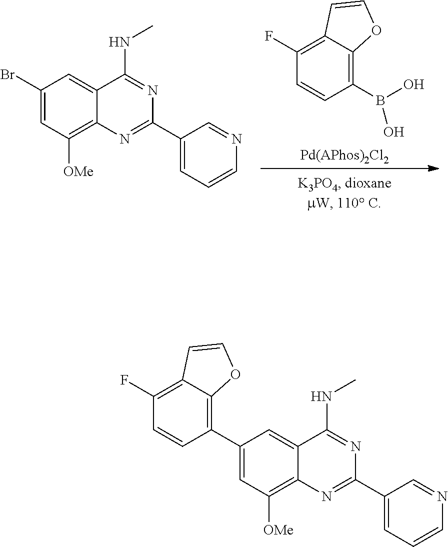 [SMILES: CNc1nc(-c2cccnc2)nc2c(OC)cc(Br)cc12 ; OB(O)c1ccc(F)c2ccoc12 ; "Pd(APhos)2Cl2" ; CNc1nc(-c2cccnc2)nc2c(OC)cc(-c3ccc(F)c4ccoc34)cc12]

### 6-(4-Fluorobenzofuran-7-yl)-8-methoxy-N-methyl-2-(pyridin-3-yl)quinazolin-4-a mine (Compound 1074) (Method RRR8)

A mixture of 6-bromo-8-methoxy-N-methyl-2-(pyridin-3-yl)quinazolin-4-amine (400 mg, 1.16 mmol), 4-fluorobenzofuran-7-ylboronic acid (236 mg, 1.39 mmol), Pd₂(APhos)₂Cl₂ (85 mg, 0.12 mmol) and K₃PO₄ (740 mg, 3.49 mmol) in dioxane/H₂O (15 mL:1.5 mL) was heated to 110° C. for 40 min by microwave irradiation. After cooling, the volatiles were removed in vacuo. The residue was purified using chromatography (silica gel, isoperatic gradient of 100% ethyl acetate). The fractions were concentrated and the resultant solid was washed with methanol and ether to give 67 mg of the desired product in 15% yield. LCMS m/z=401.1 (M+1) (Method BBB) (retention time=1.72 min). ¹H NMR (400 MHz, DMSO-d₆): δ 9.64 (s, 1H), 8.83 (d, J=8.0 Hz, 1H), 8.72 (d, J=4.4 Hz, 1H), 8.58 (d, J=4.4 Hz, 1H), 8.22 (d, J=2.0 Hz, 2H), 7.73-7.70 (m, 2H), 7.61 (dd, J=8.0, 4.8 Hz, 1H), 7.31 (t, J=8.8 Hz, 1H), 7.22 (d, J=2.4 Hz, 1H), 4.07 (s, 3H), 3.17 (d, J=4.0 Hz, 3H).

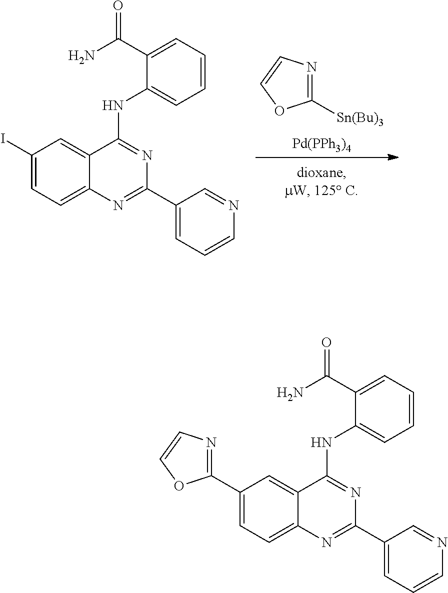

### 2-(6-(Oxazol-2-yl)-2-(pyridin-3-yl)quinazolin-4-yl)amino)benzamide (Method RRR9)

To a 10 mL microwave vial were added 2-(6-iodo-2-(pyridin-3-yl)quinazolin-4-ylamino)benzamide (0.100 g, 0.214 mmol), 2-(tri-n-butylstannyl)oxazole (0.067 ml, 0.321 mmol) and tetrakis(triphenylphosphine)palladium(0) (0.019 g, 0.016 mmol) in dioxane (1 ml) to give an orange suspension. The reaction mixture was then heated to 120° C. for 45 min using microwave irradiation. LC-MS analysis of the crude mixture showed about 40% of product formed and 55% dehalogenated starting material. The volatiles were evaporated, and the residue was purified via ISCO (silica gel, 96:4 CH₂Cl₂/MeOH, 2×4 gm columns). The fractions were concentrated and dried under vacuum to give a yellow solid. The desired product was converted to the HCl salt using 4M HCl/dioxane. LCMS m/z=409.4 (M+1) (Method CCC) (retention time=1.95 min) ¹H NMR (300 MHz, DMSO-d₆): δ 9.69 (s, 1H), 9.43 (s, 1H), 8.93 (d, J=6.2 Hz, 2H), 8.87 (d, J=7.4 Hz, 1H), 8.52 (s, 1H), 8.22-7.99 (m, 3H), 7.92 (d, J=8.2 Hz, 1H), 7.71 (t, J=6.6 Hz, 1H), 7.42 (s, 1H), 7.30 (t, J=7.5 Hz, 1H).

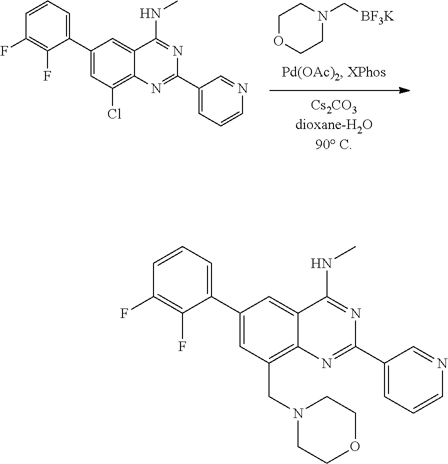

### 6-(2,3-Difluorophenyl)-N-methyl-8-(morphohnomethyl)-2-(pyridin-3-yl)quinazolin-4-amine (Method RRR5)

To a 1 dram reaction vial were added 8-chloro-6-(2,3-difluorophenyl)-N-methyl-2-(pyridin-3-yl)quinazolin-4-amine (0.050 g, 0.131 mmol), potassium 1-trifluoroboratomethylmorpholine (0.030 g, 0.144 mmol), palladium (II) acetate (0.880 mg, 3.92 mmol), 2-(dicyclohexylphosphino)-2′,4′,6′-tri-1-propyl-1,1′-biphenyl (X-Phos) (3.74 mg, 7.84 mmol), and cesium carbonate (0.128 g, 0.392 mmol) in dioxane (1 ml)/water (0.100 ml) to give a yellow solution. The reaction was heated at 90° C. overnight. LC-MS analysis of the crude mixture showed about 70% of product formed and 30% hydrolyzed starting material. After cooling, the reaction was diluted with water (10 mL) and the resultant precipitate was collected by filtration. The crude solid was purified via ISCO (silica gel, 96:4 CH₂Cl₂/MeOH, 4 gm column). The fractions were concentrated and dried under vacuum to give the desired product as an off-white powder in 34% yield. LCMS m/z=448.5 (M+1) (Method CCC) (retention time=2.15 min) ¹H NMR (300 MHz, DMSO-d₆): δ 9.66 (s, 1H), 8.79 (d, J=7.8 Hz, 1H), 8.68 (d, J=4.6 Hz, 1H), 8.59 (d, J=4.2 Hz, 1H), 8.37 (s, 1H), 8.02 (s, 1H), 7.60-7.44 (m, 3H), 7.38 (dd, J=13.7, 6.6 Hz, 1H), 4.15 (s, 2H), 3.60 (s, 4H), 3.15 (d, J=3.9 Hz, 3H), 2.53 (s, 4H).

The compounds in the following table were prepared in a manner analogous to that described in Schemes 69 and 70.

TABLE 41

| | | | | | | ¹H | Puri- | Method | | |
|---|---|---|---|---|---|---|---|---|---|---|
| | | | | | | NMR | ty | of | | Reten- |
| Num- | Starting | Starting | | Salt | | Sol- | per- | Cou- | | tion |
| ber | Material 1 | Material 2 | Product | type | ¹H NMR | vent | cent | pling | LCMS | Time |
| 1075 | 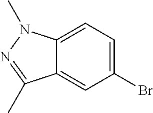 | 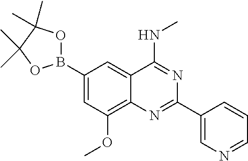 | 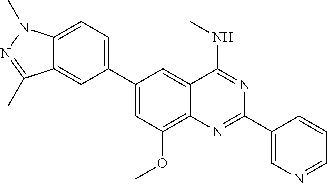 [SMILES: CNc1nc(-c2cccnc2)nc2c(OC)cc(-c3ccc4c(c3)c(C)nn4C)cc12] | | 1H-NMR (400 MHz, DMSO-d6): δ 9.64 (s, 1H), δ 8.77 (d, J = 8 Hz, 1H), 8.67 (d, J = 4.4 Hz, 1H), 8.48 (d, J = 4 Hz, 1H), 8.19 (s, 1H), 8.14 (s, 1H), 7.94 (d, 7.71 (d, J = 8.8 Hz, 1H), 7.64 (s, 1H), 7.54 (dd, J = 5.2, 8 Hz, 1H), δ 4.12 (s, 3H), δ 4.02 (s, 3H), 3.19 (d, J = 4 Hz, 1H), 2.59 (s, 3H). | DMSO | 95 | Method RRR3 | 411.2 (M + 1) | t = 1.587 min |
| 1076 | 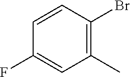 | 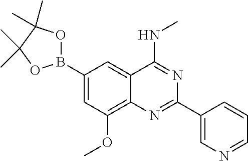 [SMILES: CNc1nc(-c2cccnc2)nc2c(OC)cc(B3OC(C)(C)C(C)(C)O3)cc12] | 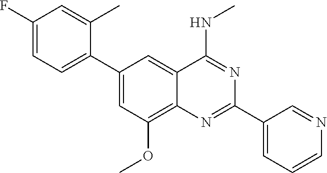 [SMILES: CNc1nc(-c2cccnc2)nc2c(OC)cc(-c3ccc(F)cc3C)cc12] | | 1H-NMR (400 MHz, DMSO-d6): δ 9.63 (d, J = 1.2 Hz, 1H), 8.76 (d, J = 4.0 Hz, 1H), 8.68 (dd, J = 4.8, 1.4 Hz, 1H), 8.35 (d, J = 4.0 Hz, 1H), 7.71 (s, 2H), 7.59 (d, J = 8.0 Hz, 1H), 7.55 (dd, J = 7.8, 4.6 Hz, 1H), 7.45 (d, J = 7.6 Hz, 1H), 7.17 (s, 1H), 3.95 (s, 3H), 3.12 (d, J = 4.0 Hz, 3H). | DMSO | 98 | Method RRR3 | 375.1 (M + 1) | t = 1.887 min |
| 1077 | 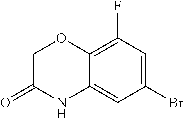 [SMILES: O=C1COc2c(F)cc(Br)cc2N1] | 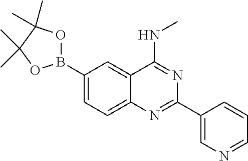 | 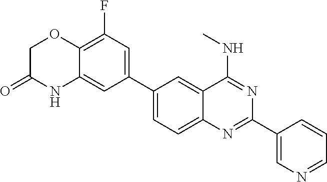 [SMILES: CNc1nc(-c2cccnc2)nc2ccc(-c3cc(F)c4c(c3)NC(=O)CO4)cc12] | | 1H-NMR (400 MHz, DMSO-d6): δ 10.40-10.20 (brs, 1H), 9.64 (d, J = 1.6 Hz, 1H), 9.04 (d, J = 3.6 Hz, 1H), 8.95-8.94 (m, 1H), 8.88 (s, 1H), 8.39 (d, J = 4.8 Hz, 1H), 8.21 (d, J = 8.4 Hz, 1H), 7.98 (d, J = 2.0 Hz, 1H), 7.87 (d, J = 2.0 Hz, 1H), 7.85 (d, J = 2.4 Hz, 1H), 7.43 (d, J = 8.8 Hz, 1H), 3.62 (s, 2H), 3.41 (s, 3H). | DMSO | 95 | Method RRR8 | 401.9 (M + 1) | t = 1.287 min |
| 1078 | 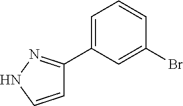 [SMILES: Brc1cccc(-c2cc[nH]n2)c1] | 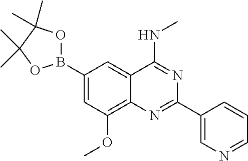 | 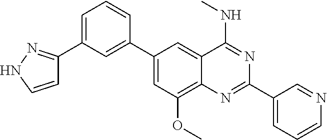 [SMILES: CNc1nc(-c2cccnc2)nc2c(OC)cc(-c3cccc(-c4cc[nH]n4)c3)cc12] | | 1H-NMR (400 MHz, DMSO-d6): δ 12.96 (s, 1H), 9.64 (s, 1H), 8.78 (d, J = 8.4 Hz, 1H), 8.68 (d, J = 4.8 Hz, 1H), 8.55 (s, 1H), 8.25 (s, 1H), 8.16 (s, 1H), 7.85-7.80 (m, 3H), 7.59-7.53 (m, 3H), 6.87 (s, 1H), 4.10 (s, 3H), 3.18 (s, 3H). | DMSO | 95 | Method RRR3 | 409.0 (M + 1) | t = 1.519 min |
| 1079 | 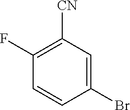 | 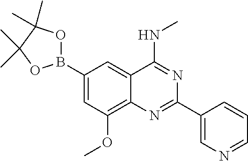 | 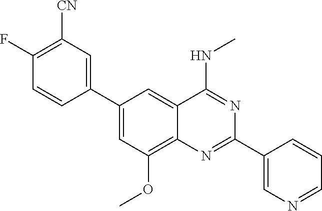 [SMILES: CNc1nc(-c2cccnc2)nc2c(OC)cc(-c3ccc(F)c(C#N)c3)cc12] | | 1H-NMR (400 MHz, DMSO-d6): δ 9.62 (s, 1H), 8.76 (d, J = 6.8 Hz, 1H), 8.68 (s, 1H), 8.47 (s, 1H), 8.31 (s, 1H), 8.15 (s, 1H), 7.73 (t, J = 8.8 Hz, 1H), 7.62 (s, 1H), 7.55-7.52 (m, 1H), 4.08 (s, 3H), 3.18 (d, J = 2.4 Hz, 3H). | DMSO | 95 | Method RRR3 | 386.1 (M + 1) | t = 1.646 min |
| 1080 | 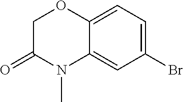 | 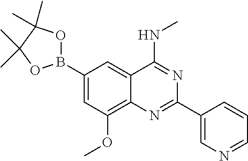 | 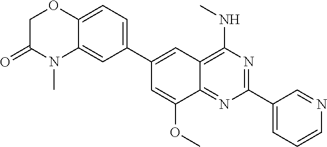 [SMILES: CNc1nc(-c2cccnc2)nc2c(OC)cc(-c3ccc4c(c3)N(C)C(=O)CO4)cc12] | | 1H-NMR (400 MHz, DMSO-d6): δ 9.60 (s, 1H), 9.04 (d, J = 8.0 Hz, 1H), 8.92-8.87 (m, 2H), 8.11 (d, J = 1.6 Hz, 1H), 7.88 (dd, J = 5.2, 8.0 Hz, 1H), 7.64 (d, J = 1.6 Hz, 1H), 7.55-7.53 (m, 2H), 7.18 (d, J = 7.6 Hz, 1H), 4.74 (s, 2H), 4.11 (s, 3H), 3.22 (d, J = 4.8 Hz, 3H). | DMSO | 95 | Method RRR3 | 428.4 (M + 1) | t = 1.065 min |
| 1081 | 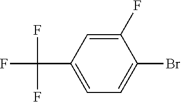 [SMILES: Fc1cc(C(F)(F)F)ccc1Br] | 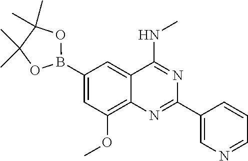 | 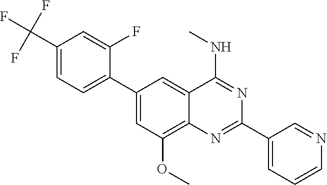 [SMILES: CNc1nc(-c2cccnc2)nc2c(OC)cc(-c3ccc(C(F)(F)F)cc3F)cc12] | | 1H-NMR (400 MHz, DMSO-d6): δ 9.64 (s, 1H), 8.99 (d, J = 8.4 Hz, 1H), 8.83-8.82 (m, 1H), 8.76 (s, 1H), 8.08 (s, 1H), 7.97-7.90 (m, 2H), 7.80-7.78 (m, 2H), 7.53 (s, 1H), 4.04 (s, 3H), 3.18 (s, 3H). | DMSO | 95 | Method RRR3 | 429.0 (M + 1) | t = 1.747 min |
| 1082 | 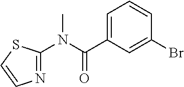 | 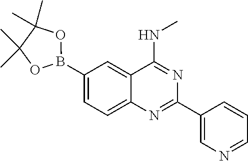 | 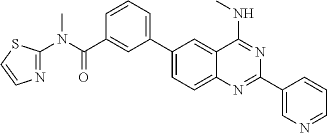 [SMILES: CNc1nc(-c2cccnc2)nc2ccc(-c3cccc(C(=O)N(C)c4nccs4)c3)cc12] | | 1H-NMR (400 MHz, DMSO-d6): δ 9.66 (s, 1H), 8.80 (dd, J = 8.0, 1.6 Hz, 1H), 8.74-8.65 (m, 4H), 8.31 (d, J = 8.0 Hz, 1H), 8.18 (d, J = 8.8 Hz, 1H), 8.00 (d, J = 7.6 Hz, 1H), 7.90 (d, J = 8.4 Hz, 1H), 7.67-7.54 (m, 3H), 7.10-7.09 (m, 1H), 3.87 (s, 3H), 3.20 (d, J = 3.6 Hz, 3H). | DMSO | 95 | Method RRR3 | 453.0 (M + 1) | t = 1.658 min |
| 1083 | 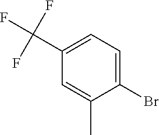 | 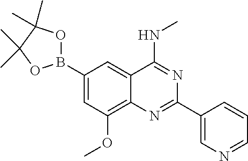 | 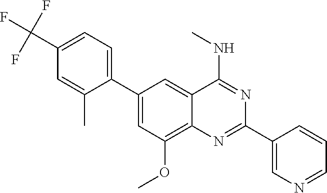 [SMILES: CNc1nc(-c2cccnc2)nc2c(OC)cc(-c3ccc(C(F)(F)F)cc3C)cc12] | | 1H-NMR (400 MHz, DMSO-d6): δ 9.63 (d, J = 1.2 Hz, 1H), 8.77 (dd, J = 8.0, 1.6 Hz, 1H), 8.68 (dd, J = 5.2, 1.2 Hz, 1H), 8.38 (d, J = 4.4 Hz, 1H), 7.78-7.75 (m, 2H), 7.67 (d, J = 8.0 Hz, 1H), 7.59-7.53 (m, 2H), 7.29 (s, 1H), 4.02 (s, 3H), 3.13 (d, J = 4.4 Hz, 3H), 1.99 (s, 3H). | DMSO | 95 | Method RRR3 | 425. 1 (M + 1) | t = 1.658 min |
| 1084 | 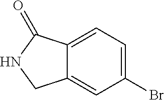 [SMILES: O=C1NCc2cc(Br)ccc21] | 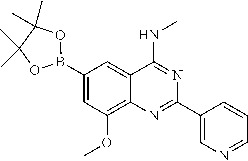 | 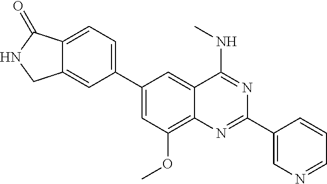 [SMILES: CNc1nc(-c2cccnc2)nc2c(OC)cc(-c3ccc4c(c3)CNC4=O)cc12] | | 1H-NMR (400 MHz, DMSO-d6): δ 9.63 (d, J = 2.0 Hz, 1H), 8.78-8.75 (m, 1H), 8.69-8.55 (m, 3H), 8.20 (s, 1H), 8.09 (s, 1H), 8.01 (d, J = 8 Hz, 1H), 7.82 (d, J = 8 Hz, 1H), 7.62 (d, J = 1.2 Hz, 1H), 7.55 (dd, J = 4.8, 8 Hz, 1H), 4.49 (s, 2H), 4.09 (s, 3H), 3.18 (d, J = 4.4 Hz, 3H). | DMSO | 95 | Method RRR3 | 398.1 (M + 1) | t = 1.399 min |
| 1085 | 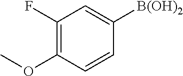 | 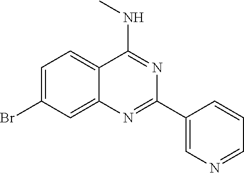 | 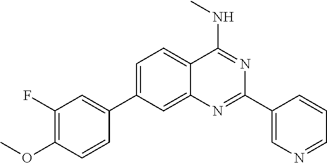 [SMILES: CNc1nc(-c2cccnc2)nc2cc(-c3ccc(OC)c(F)c3)ccc12] | | 1H-NMR (400 MHz, DMSO-d6): δ 9.66 (s, 1H), 8.78 (d, J = 7.6 Hz, 1H), 8.69 (d, J = 4.8 Hz, 1H), 8.50 (d, J = 4.4 Hz, 1H), 8.28 (d, J = 8.8 Hz, 1H), 8.03 (s, 1H), 7.87-7.79 (m, 2H), 7.72 (d, J = 8.8 Hz, 1H), 7.55 (dd, J = 5.2, 8 Hz, 1H), 7.31 (t, J = 8.8 Hz, 1H), 3.92 (s, 3H), 3.17 (d, J = 4.4 Hz, 3H). | DMSO | 95 | Method RRR3 | 361.1 (M + 1) | t = 1.674 min |
| 1086 | 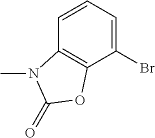 | 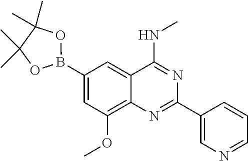 | 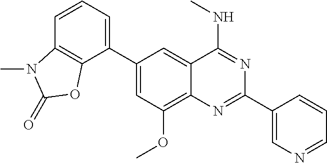 [SMILES: CNc1nc(-c2cccnc2)nc2c(OC)cc(-c3cccc4c3oc(=O)n4C)cc12] | | 1H-NMR (400 MHz, DMSO-d6): δ 9.63 (d, J = 1.6 Hz, 1H), 8.79-8.76 (m, 1H), 8.68 (dd, J = 4.8, 1.6 Hz, 1H), 8.54-8.51 (m, 1H), 8.14 (d, J = 1.6 Hz, 1H), 7.62 (d, J = 1.2 Hz, 1H), 7.57-7.52 (m, 2H), 7.40 (t, J = 8.0 Hz, 1H), 7.31 (d, J = 6.8 Hz, 1H), 4.04 (s, 3H), 3.53 (d, J = 5.6 Hz, 3H), 3.17 (d, J = 4.8 Hz, 3H). | DMSO | 95 | Method RRR3 | 414.1 (M + 1) | t = 1.683 min |
| 1087 | 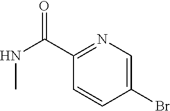 | 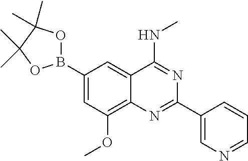 | 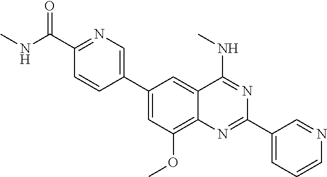 [SMILES: CNC(=O)c1ccc(-c2cc(OC)c3nc(-c4cccnc4)nc(NC)c3c2)cn1] | | 1H-NMR (400 MHz, DMSO-d6): δ 9.64 (s, 1H), 9.15 (s, 1H), 8.79-8.69 (m, 3H), 8.56-8.47 (m, 2H), 8.26-8.17 (m, 2H), 8.67-7.56 (m, 2H), 4.10 (s, 3H), 3.19 (s, 3H), 2.89 (s, 3H). | DMSO | 95 | Method RRR3 | 401.0 (M + 1) | t = 1.427 min |
| 1088 | 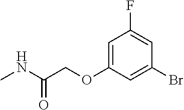 | 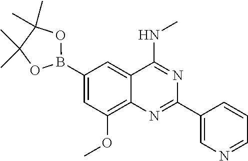 | 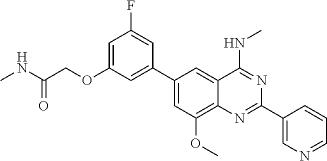 [SMILES: CNC(=O)COc1cc(F)cc(-c2cc(OC)c3nc(-c4cccnc4)nc(NC)c3c2)c1] | | 1H-NMR (400 MHz, DMSO-d6): δ 9.63 (s, 1H), 8.76 (d, J = 8.4 Hz, 1H), 8.68 (d, J = 4.4 Hz, 1H), 8.52-8.51 (m, 1H), 8.14-8.11 (m, 2H), 7.56-7.53 (m, 2H), 7.40-7.36 (m, 2H), 6.91 (d, J = 11.2 Hz, 1H), 4.63 (s, 2H), 4.08 (s, 3H), 3.18 (s, 3H), 2.69 (s, 3H). | DMSO | 95 | Method RRR3 | 448.0 (M + 1) | t = 1.535 min |
| 1089 | 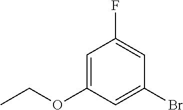 | 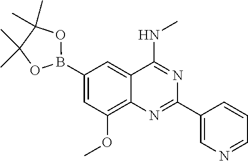 | 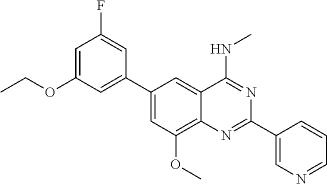 [SMILES: CCOc1cc(F)cc(-c2cc(OC)c3nc(-c4cccnc4)nc(NC)c3c2)c1] | | 1H-NMR (400 MHz, DMSO-d6): δ 9.63 (s, 1H), 8.81 (d, J = 8 Hz, 1H), 8.67 (d, J = 3.6 Hz, 1H), 8.52 (d, J = 5.2 Hz, 1H), 8.14 (s, 1H), 7.56-7.53 (m, 2H), 7.35-7.31 (m, 2H), 6.89 (d, J = 10.4 Hz, 1H), 4.19-4.08 (m, 2H), 4.08 (s, 3H), 3.17 (s, 3H), 1.39 (t, 3H). | DMSO | 95 | Method RRR3 | 405.0 (M + 1) | t = 1.774 min |
| 1090 | 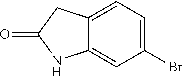 [SMILES: O=C1Cc2ccc(Br)cc2N1] | 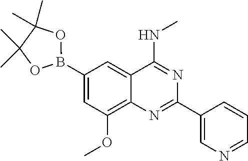 | 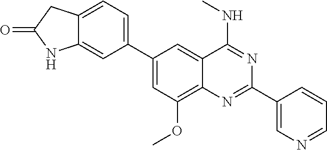 [SMILES: CNc1nc(-c2cccnc2)nc2c(OC)cc(-c3ccc4c(c3)NC(=O)C4)cc12] | | 1H-NMR (400 MHz, DMSO-d6): δ 10.58 (s, 1H), 9.63 (d, J = 1.6 Hz, 1H), 8.77-8.75 (m, 1H), 8.68-8.66 (m, 1H), 8.54 (d, J = 4.4 Hz, 1H), 8.06 (s, 1H), 7.56-7.35 (m, 4H), 7.23 (s, 1H), 4.06 (s, 3H), 3.56 (s, 2H), 3.17 (d, J = 4 Hz, 3H). | DMSO | 95 | Method RRR3 | 398.0 (M + 1) | t = 1.445 min |
| 1091 | 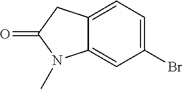 | 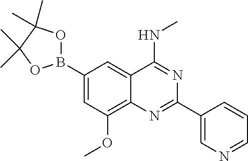 | 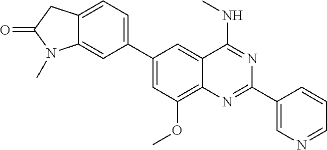 [SMILES: CNc1nc(-c2cccnc2)nc2c(OC)cc(-c3ccc4c(c3)N(C)C(=O)C4)cc12] | | 1H-NMR (400 MHz, DMSO-d6): δ 9.64 (s, 1H), 8.77 (d, J = 7.6 Hz, 1H), 8.68 (d, J = 3.6 Hz, 1H), 8.50 (d, J = 4 Hz, 1H), 8.12 (s, 1H), 7.58-7.52 (m, 3H), 7.44-7.39 (m, 2H), 4.09 (s, 3H), 3.63 (s, 2H), 3.29 (s, 3H), 3.19 (d, J = 4.4 Hz, 3H). | DMSO | 95 | Method RRR3 | 412.0 (M + 1) | t = 1.483 min |
| 1092 | 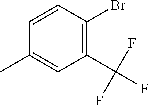 | 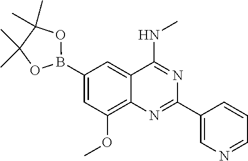 | 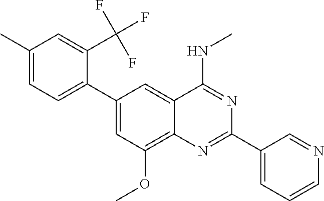 [SMILES: CNc1nc(-c2cccnc2)nc2c(OC)cc(-c3ccc(C)cc3C(F)(F)F)cc12] | | 1H-NMR (400 MHz, DMSO-d6): δ 9.63 (d, J = 1.2 Hz, 1H), 8.76 (d, J = 4.0 Hz, 1H), 8.68 (dd, J = 4.8, 1.4 Hz, 1H), 8.35 (d, J = 4.0 Hz, 1H), 7.71 (s, 2H), 7.59 (d, J = 8.0 Hz, 1H), 7.55 (dd, J = 7.8, 4.6 Hz, 1H), 7.45 (d, J = 7.6 Hz, 1H), 7.17 (s, 1H), 3.95 (s, 3H), 3.12 (d, J = 4.0 Hz, 3H). | DMSO | 100 | Method RRR3 | 425.0 (M + 1) | t = 1.945 min |
| 1093 | 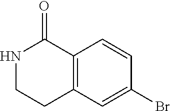 [SMILES: O=C1NCCc2cc(Br)ccc21] | 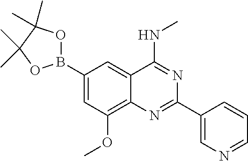 | 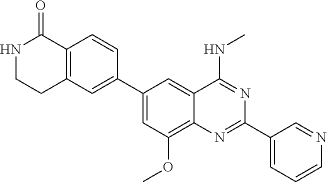 [SMILES: CNc1nc(-c2cccnc2)nc2c(OC)cc(-c3ccc4c(c3)CCNC4=O)cc12] | | 1H-NMR (400 MHz, DMSO-d6): δ 9.63 (d, J = 1.2 Hz, 1H), 8.76 (d, J = 8.0 Hz, 1H), 8.68 (dd, J = 4.4, 1.2 Hz, 1H), 8.54 (d, J = 4.4 Hz, 1H), 8.17 (s, 1H), 8.00 (s, 1H), 7.97 (d, J = 8.0 Hz, 1H), 7.86 (d, J = 8.4 Hz, 1H), 7.83 (s, 1H), 7.59 (s, 1H), 7.54 (dd, J = 8.0, 4.8 Hz, 1H), 4.08 (s, 3H), 3.46-3.43 (m, 2H), 3.17 (d, J = 4.4 Hz, 3H), 3.03 (t, J = 6.4 Hz, 2H). | DMSO | 100 | Method RRR3 | 412.0 (M + 1) | t = 1.412 min |
| 1094 | 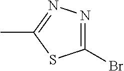 | 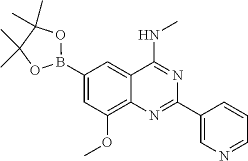 | 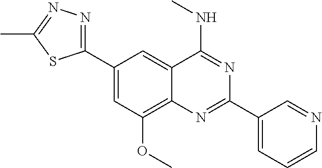 [SMILES: CNc1nc(-c2cccnc2)nc2c(OC)cc(-c3nnc(C)s3)cc12] | | ¹H-NMR (400 MHz, DMSO-d6): δ 9.62 (s, 1H), 8.74-8.69 (m, 2H), 8.30 (s, 1H), 7.76 (s, 1H), 7.55-7.54 (m, 1H), 4.05 (s, 3H), 3.24 (s, 3H), 2.82 (s, 3H). | DMSO | 95 | Method RRR3 | 365.4 (M + 1) | t = 1.420 min |
| 1095 | 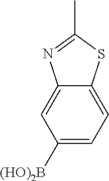 | 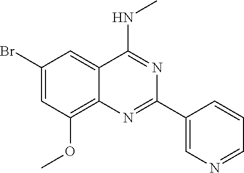 | 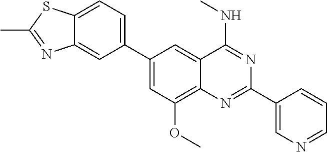 [SMILES: CNc1nc(-c2cccnc2)nc2c(OC)cc(-c3ccc4sc(C)nc4c3)cc12] | | 1H-NMR (400 MHz, DMSO-d6): δ 9.64 (s, 1H), 8.77 (d, J = 8 Hz, 1H), 8.68-8.67 (m, 1H), 8.56 (s, 1H), 8.52 (d, J = 4.4 Hz, 1H), 8.20 (s, 1H), 8.07-8.01 (m, 2H), 7.66 (s, 1H), 7.56-7.53 (m, 2H), 4.10 (s, 3H), 3.18 (s, 3H), 2.85 (s, 3H). | DMSO | 95 | Method RRR3 | 414.1 (M + 1) | t = 1.610 min |
| 1096 | 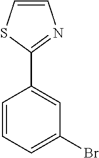 [SMILES: Brc1cccc(-c2nccs2)c1] | 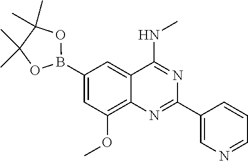 | 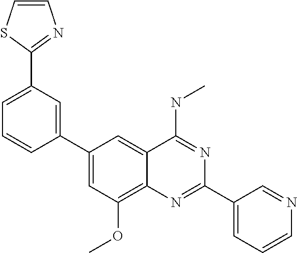 [SMILES: CNc1nc(-c2cccnc2)nc2c(OC)cc(-c3cccc(-c4nccs4)c3)cc12] | | 1H-NMR (400 MHz, MeOD): δ 9.48 (s, 1H), 8.90-8.88 (m, 2H), 8.41 (s, 1H), 8.15 (s, 1H), 8.01 (d, J = 8 Hz, 1H), 7.97 (s, 1H), 7.95 (d, J = 8.0 Hz, 1H), 7.87-7.84 (m, 2H), 7.72-7.65 (m, 2H), 4.26 (s, 3H), 3.42 (s, 3H) | MeOD | 95 | Method RRR3 | 425.9 (M + 1) | t = 1.710 min |
| 1097 | 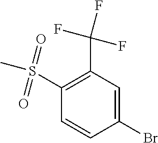 | 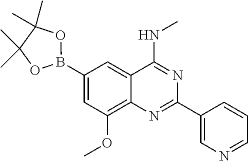 | 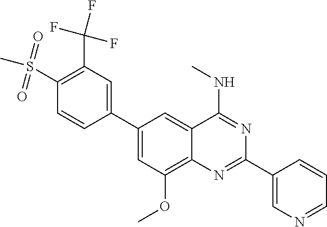 [SMILES: CNc1nc(-c2cccnc2)nc2c(OC)cc(-c3ccc(S(C)(=O)=O)c(C(F)(F)F)c3)cc12] | | 1H-NMR (400 MHz, DMSO-d6): δ 9.64 (d, J = 1.8 Hz, 1H), 8.74-8.64 (m, 1H), 8.50-8.24 (m, 5H), 7.69 (d, J = 1.4 Hz, 1H), 7.56 (dd, J = 7.8, 4.8 Hz, 1H), 4.10 (s, 3H), 3.38 (s, 3H), 3.20 (s, 3H). | DMSO | 95 | Method RRR3 | 488.9 (M + 1) | t = 1.594 min |
| 1098 | 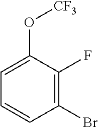 [SMILES: Fc1c(Br)cccc1OC(F)(F)F] | 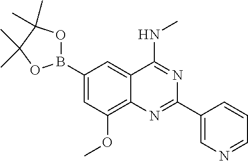 | 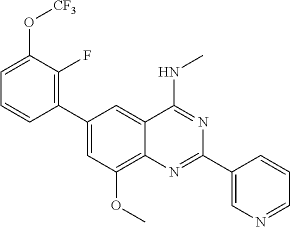 [SMILES: CNc1nc(-c2cccnc2)nc2c(OC)cc(-c3cccc(OC(F)(F)F)c3F)cc12] | | 1H-NMR (400 MHz, DMSO-d6): δ 9.64 (s, 1H), 8.77 (d, J = 7.6 Hz, 1H), 8.69 (d, J = 3.6 Hz, 1H), 8.50 (d, J = 3.2 Hz, 1H), 8.01 (s, 1H), 7.76 (t, J = 7.2 Hz, 1H), 7.65 (t, J = 7.2 Hz, 1H), 7.57-7.44 (m, 3H), 4.03 (s, 3H), 3.15 (d, J = 4 Hz, 3H). | DMSO | 95 | Method RRR3 | 445.1 (M + 1) | t = 1.791 min |
| 1099 | 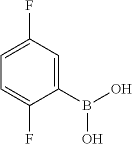 [SMILES: OB(O)c1cc(F)ccc1F] | 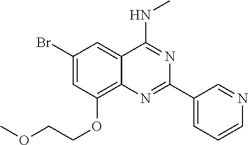 [SMILES: CNc1nc(-c2cccnc2)nc2c(OCCOC)cc(Br)cc12] | 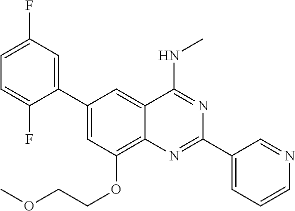 [SMILES: CNc1nc(-c2cccnc2)nc2c(OCCOC)cc(-c3cc(F)ccc3F)cc12] | | 1H-NMR (400 MHz, DMSO-d6): δ 9.63 (s, 1H), 9.39 (s, 1H), 8.77 (dd, J = 1.6, 3.6 Hz), 8.76-8.68 (m, 1H), 8.48 (d, J = 8 Hz), 7.97 (s, 1H), 7.78-7.72 (m, 1H), 7.57-7.48 (m, 1H), 7.47-7.31 (m, 2H), 7.31-7.27 (m, 1H), 4.38 (dd, J = 4.4, 6.0 Hz, 2H), 3.85-3.83 (m, 2H), 3.44 (s, 3H), 3.15 (dd, J = 4.8 Hz). | DMSO | 95 | Method RRR3 | 423.4 (M + 1) | t = 1.666 min |
| 1100 | 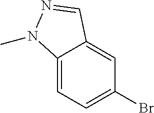 | 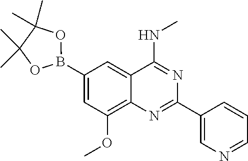 | 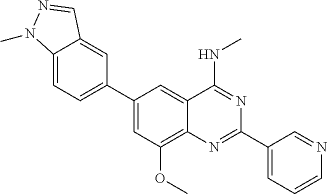 [SMILES: CNc1nc(-c2cccnc2)nc2c(OC)cc(-c3ccc4c(cnn4C)c3)cc12] | 3HCl | 1H-NMR (400 MHz, DMSO-d6): δ 9.55 (s, 1H), 9.41 (s, 1H), 9.16 (s, 1H), 8.97 (dd, J = 5.2, 0.8 Hz, 1H), 8.32 (s, 1H), 8.31 (s, 1H), 8.17 (s, 1H), 8.02-7.99 (m, 2H), 7.83-7.78 (m, 2H), 4.15 (s, 3H), 4.12 (s, 3H), 3.24 (d, J = 4.4 Hz, 3H). | DMSO | 95 | Method RRR3 | 396.9 (M + 1) | t = 1.245 min |
| 1101 | 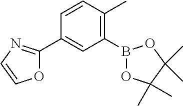 | 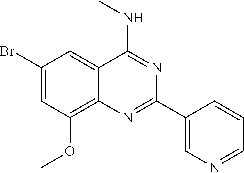 | 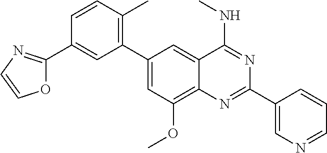 [SMILES: CNc1nc(-c2cccnc2)nc2c(OC)cc(-c3cc(-c4ncco4)ccc3C)cc12] | 2HCl | 1H-NMR (400 MHz, DMSO-d6): δ 9.56 (s, 1H), 9.15-9.07 (m, 1H), 8.95 (s, 1H), 8.24 (s, 1H), 7.98-7.95 (m, 4H), 7.56 (d, J = 4.0 Hz, 1H), 7.47 (s, 1H), 7.40 (s, 1H), 4.06 (s, 3H), 3.19 (s, 3H), 2.40 (s, 3H). | DMSO | 98 | Method RRR3 | 424.0 (M + 1) | t = 1.648 min |
| 1102 | 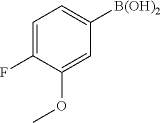 | 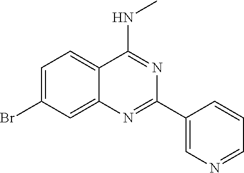 | 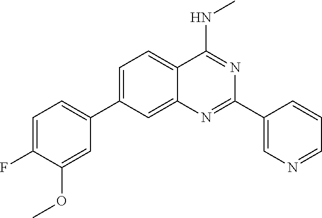 [SMILES: CNc1nc(-c2cccnc2)nc2cc(-c3ccc(F)c(OC)c3)ccc12] | | 1H-NMR (400 MHz, DMSO-d6): δ 9.65 (d, J = 1.6 Hz, 1H), 8.78 (d, J = 8.0 Hz, 1H), 8.77-8.68 (m, 1h), 8.54-8.52 (m, 1H), 8.32-8.30 (m, 1H), 8.08 (d, J = 1.6 Hz, 1H), 7.89-7.86 (m, 1H), 7.63-7.60 (m, 2H), 7.54-7.30 (m, 2H), 3.99 (s, 3H), 3.18 (d, J = 4.8 Hz, 3H). | DMSO | 98 | Method RRR3 | 361.0 (M + 1) | t = 1.768 min |
| 1103 | 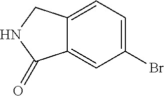 [SMILES: O=C1NCc2ccc(Br)cc21] | 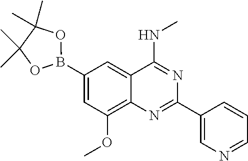 | 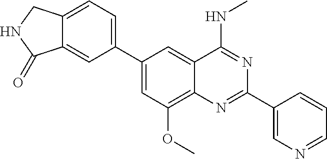 [SMILES: CNc1nc(-c2cccnc2)nc2c(OC)cc(-c3ccc4c(c3)C(=O)NC4)cc12] | | 1H-NMR (400 MHz, DMSO-d6): δ 9.64 (s, 1H), 8.77 (d, J = 8.4 Hz, 1H), 8.68-8.06 (m, 3H), 8.23-8.13 (m, 3H), 7.73 (d, J = 7.6 Hz, 1H), 7.63 (s, 1H), 7.55 (dd, J = 5.2, 8 Hz, 3H), 4.47 (s, 2H), 4.10 (s, 3H), 3.18 (d, J = 4 Hz, 3H). | DMSO | 95 | Method RRR3 | 398.1 (M + 1) | t = 1.416 min |
| 1104 | 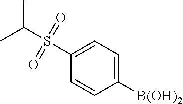 | 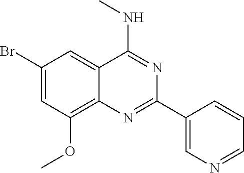 | 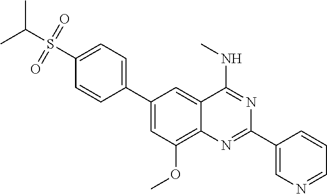 [SMILES: CNc1nc(-c2cccnc2)nc2c(OC)cc(-c3ccc(S(=O)(=O)C(C)C)cc3)cc12] | | 1H-NMR (400 MHz, DMSO-d6): δ 9.64 (s, 1H), 8.77 (d, J = 8.4 Hz, 1H), 8.68 (d, J = 4.8 Hz, 1H), 8.58 (d, J = 4 Hz, 1H), 8.24 (s, 1H), 8.17 (d, J = 8 Hz, 2H), 8.00 (d, J = 8.4 Hz, 2H), 7.64 (s, 1H), 7.55 (dd, J = 4.8, 7.6 Hz, 1H), 4.09 (s, 3H), 3.52-3.48 (m, 1H), 3.18 (d, J = 4.4 Hz, 3H), 1.21 (d, J = 6.8 Hz, 6H), | DMSO | 95 | Method RRR3 | 449.2 (M + 1) | t = 1.572 min |
| 1105 | 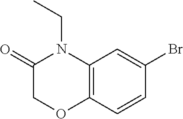 | 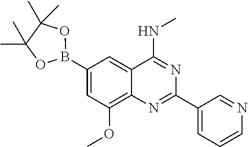 | 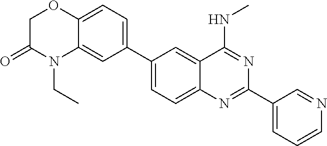 [SMILES: CCN1C(=O)COc2ccc(-c3ccc4nc(-c5cccnc5)nc(NC)c4c3)cc21] | | 1H NMR (300 MHz, DMSO) δ 9.63 (d, J = 1.2 Hz, 1H), 8.76 (d, J = 8.0 Hz, 1H), 8.69 (dd, J = 11.1, 5.0 Hz, 2H), 8.57 (s, 1H), 8.49 (t, J = 5.5 Hz, 1H), 8.23 (d, J = 2.3 Hz, 1H), 8.10 (d, J = 8.7 Hz, 1H), 7.93 (dd, J = 8.5, 2.4 Hz, 1H), 7.83 (d, J = 8.7 Hz, 1H), 7.53 (dd, J = 7.9, 4.8 Hz, 1H), 7.17 (d, J = 8.5 Hz, 1H), 4.34 (t, J = 4.5 Hz, 2H), 3.37 (d, J = 4.6 Hz, 2H), 3.17 (d, J = 4.1 Hz, 3H). | DMSO | 95 | Method RRR7 | 412.3 (M + 1) | t = 2.08 min |
| 1106 | 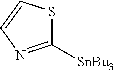 | 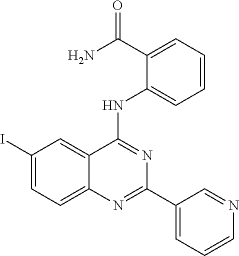 [SMILES: NC(=O)c1ccccc1Nc1nc(-c2cccnc2)nc2ccc(I)cc12] | 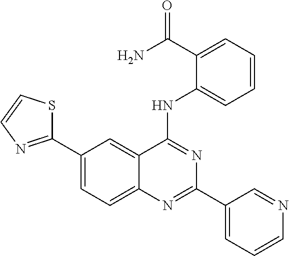 [SMILES: NC(=O)c1ccccc1Nc1nc(-c2cccnc2)nc2ccc(-c3nccs3)cc12] | 2HCl | 1H NMR (300 MHz, MeOH) δ 8.87 (s, 1H), 8.58 (d, J = 5.2 Hz, 1H), 8.15-7.97 (m, 4H), 7.69 (d, J = 7.9 Hz, 1H), 7.30 (t, J = 5.4 Hz, 1H), 7.25-7.14 (m, 3H), 7.11 (d, J = 7.8 Hz, 1H), 6.99-6.84 (m, 2H), 6.48 (t, J = 6.5 Hz, 1H). | DMSO | 95 | Method RRR9 | 425.3 (M + 1) | t = 2.11 min |
| 1107 | 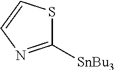 | 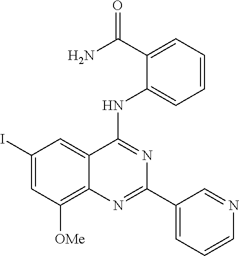 [SMILES: COc1cc(I)cc2c(Nc3ccccc3C(N)=O)nc(-c3cccnc3)nc12] | 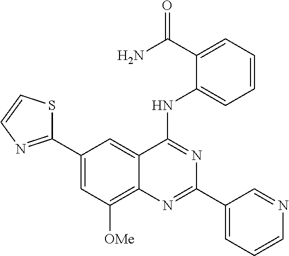 [SMILES: COc1cc(-c2nccs2)cc2c(Nc3ccccc3C(N)=O)nc(-c3cccnc3)nc12] | 2HCl | 1H NMR (300 MHz, CD3OD) δ 9.52 (s, 1H), 9.23 (d, J = 8.1 Hz, 1H), 8.83 (d, J = 5.5 Hz, 1H), 8.77 (d, J = 8.2 Hz, 1H), 8.10-8.00 (m, 1H), 7.93 (s, 1H), 7.88 (d, J = 3.2 Hz, 1H), 7.80 (d, J = 7.9 Hz, 1H), 7.67 (d, J = 3.2 Hz, 1H), 7.64 (s, 1H), 7.58 (t, J = 7.8 Hz, 1H), 7.16 (t, 1 = 7.5 Hz, 1H), 3.88 (s, 4H). | DMSO | 95 | Method RRR9 | 455.3 (M + 1) | t = 1.96 min |
| 1108 | 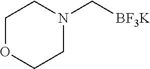 | 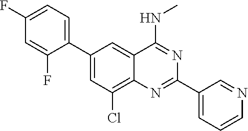 | 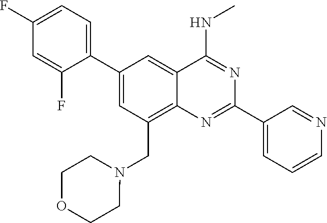 [SMILES: CNc1nc(-c2cccnc2)nc2c(CN3CCOCC3)cc(-c3ccc(F)cc3F)cc12] | | 1H NMR (300 MHz, DMSO) δ 9.66 (d, J = 1.2 Hz, 1H), 8.79 (dt, J = 7.5, 1.7 Hz, 1H), 8.68 (dd, J = 4.8, 1.7 Hz, 1H), 8.55 (d, J = 4.5 Hz, 1H), 8.31 (s, 1H), 7.98 (s, 1H), 7.71 (dt, J = 8.9, 6.7 Hz, 1H), 7.55 (dd, J = 7.9, 4.8 Hz, 1H), 7.44 (ddd, J = 11.6, 9.5, 2.5 Hz, 1H), 7.29 (dd, J = 8.4, 6.0 Hz, 1H), 4.15 (s, 2H), 3.64-3.55 (m, 4H), 3.14 (d, J = 4.2 Hz, 3H), 2.53 (s, J = 3.7 Hz, 4H). | DMSO | 95 | Method RRR5 | 448.3 (M + 1) | t = 2.15 min |
| 1109 | 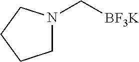 | 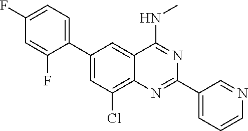 | 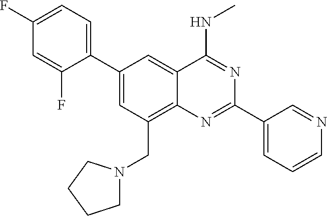 [SMILES: CNc1nc(-c2cccnc2)nc2c(CN3CCCC3)cc(-c3ccc(F)cc3F)cc12] | | 1H NMR (300 MHz, CD3OD) δ 9.68 (s, 1H), 8.96 (d, J = 6.6 Hz, 1H), 8.61 (d, J = 4.8 Hz, 1H), 8.23 (s, 1H), 8.01 (s, 1H), 7.56 (dd, J = 7.9, 4.9 Hz, 1H), 7.41 (t, J = 7.1 Hz, 1H), 7.30 (dd, J = 11.7, 5.7 Hz, 2H), 4.41 (s, 2H), 3.31 (d, J = 1.6 Hz, 3H), 2.82 (s, 4H), 1.86 (s, 4H). | DMSO | 95 | Method RRR5 | 432.3 (M + 1) | t = 1.79 min |
| 1110 | 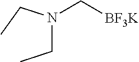 | 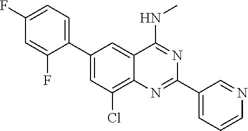 | 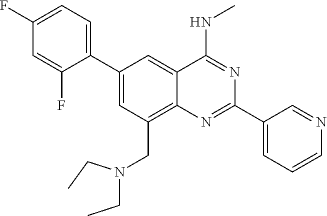 [SMILES: CCN(CC)Cc1cc(-c2ccc(F)cc2F)cc2c(NC)nc(-c3cccnc3)nc12] | | 1H NMR (300 MHz, CD3OD) δ 9.65 (d, J = 0.8 Hz, 1H), 8.97-8.88 (m, 1H), 8.59 (dd, J = 4.9, 1.7 Hz, 1H), 8.16 (s, 1H), 8.00 (d, J = 1.7 Hz, 1H), 7.53 (dd, J = 7.5, 5.4 Hz, 1H), 7.39 (ddd, J = 6.9, 5.1, 2.0 Hz, 1H), 7.35-7.22 (m, 2H), 4.26 (s, 2H), 3.20 (d, J = 0.9 Hz, 3H), 2.70 (q, J = 7.1 Hz, 4H), 1.15 (t, J = 7.1 Hz, 6H). | DMSO | 95 | Method RRR5 | 434.8 (M + 1) | t = 1.79 min |
| 1111 | 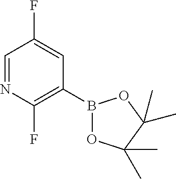 | 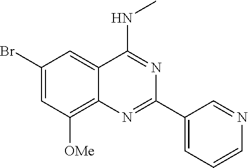 | 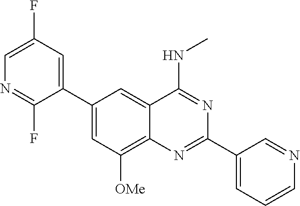 [SMILES: CNc1nc(-c2cccnc2)nc2c(OC)cc(-c3cc(F)cnc3F)cc12] | | 1H NMR (300 MHz, DMSO) δ 9.64 (s, 1H), 8.77 (dd, J = 8.0, 1.6 Hz, 1H), 8.69 (dd, J = 4.5, 1.5 Hz, 1H), 8.58 (s, 1H), 8.45 (s, 1H), 8.15 (s, 1H), 7.70-7.62 (m, 1H), 7.56 (d, J = 8.8 Hz, 2H), 4.04 (s, 3H), 3.16 (d, J = 4.0 Hz, 3H). | DMSO | 95 | Method RRR4 | 380.3 (M + 1) | t = 1.92 min |

### Biological Testing:

### STEP46 Biochemical Assays

Serial dilutions of compounds were performed in 100% DMSO and 1 uL of compounds were dispensed into 384-well black polystyrene plates (Corning, N.Y.). Compounds were incubated with 24 uL of buffer containing 50 mM Hepes, 1 mM DTT, 0.02% Brij35, 1 ng/well purified STEP46 enzyme for 30 mM at room temperature. The reaction was initiated by addition of 25 uL of DiFMUP (6,8-difluoro-4-methylumbelliferyl phosphate) (InVitrogen, CA) with a final concentration of 10 μM and incubated at 27° C. for 90 mM Final DMSO concentration is 2%. Plates were read with florescence intensity at excitation/emission of 360/460 nm using a PheraStar plate reader (BMG Labtech, NC).

### Data Analysis

Data were expressed as percentage (%) inhibition of enzyme activity. 0% inhibition is defined as the RFUs (relative fluorescence units) in the absence of compounds and 100% inhibition is defined as RFUs in the absence of STEP46 enzyme. IC₅₀ values of compounds with inhibitory activity against STEP46 were determined by GraphPad Prism (version 4.03) using four parameter logistic equation. Some compounds act as activators. For compounds showing STEP46 enzymatic activation, data are represented as percentage of inhibition but with negative values at three representative concentrations (25, 50 and 100 uM).

Compounds 1-1111 show either inhibition or activation >50% at 100 uM, 50 or 25 uM.

| | STEP IC50 |
|---|---|
| Number | (μM) |
| 4 | ++ |
| 5 | ++ |
| 6 | + |
| 8 | + |
| 12 | ++ |
| 13 | ++ |
| 14 | ++ |
| 16 | ++ |
| 17 | ++ |
| 19 | ++ |
| 20 | ++ |
| 21 | + |
| 22 | ++ |
| 23 | ++ |
| 24 | ++ |
| 25 | ++ |
| 31 | ++ |
| 32 | + |
| 35 | ++ |
| 37 | ++ |
| 39 | ++ |
| 40 | + |
| 41 | ++ |
| 43 | +++ |
| 45 | + |
| 46 | ++ |
| 47 | ++ |
| 48 | ++ |
| 49 | + |
| 50 | ++ |
| 51 | + |
| 52 | ++ |
| 55 | ++ |
| 56 | ++ |
| 57 | ++ |
| 61 | ++ |
| 62 | ++ |
| 65 | ++ |
| 66 | ++ |
| 67 | ++ |
| 69 | +++ |
| 70 | +++ |
| 71 | ++ |
| 72 | ++ |
| 73 | ++ |
| 74 | ++ |
| 75 | ++ |
| 76 | ++ |
| 78 | ++ |
| 79 | ++ |
| 80 | + |
| 81 | ++ |
| 82 | ++ |
| 83 | +++ |
| 86 | ++ |
| 87 | +++ |
| 88 | ++ |
| 90 | +++ |
| 91 | ++ |
| 93 | ++ |
| 94 | ++ |
| 95 | ++ |
| 96 | ++ |
| 97 | ++ |
| 98 | ++ |
| 99 | +++ |
| 100 | ++ |
| 101 | +++ |
| 102 | ++ |
| 103 | ++ |
| 104 | +++ |
| 106 | +++ |
| 108 | ++ |
| 109 | ++ |
| 110 | +++ |
| 111 | + |
| 114 | +++ |
| 115 | +++ |
| 117 | + |
| 124 | +++ |
| 136 | ++ |
| 137 | +++ |
| 138 | ++ |
| 141 | ++ |
| 142 | ++ |
| 143 | ++ |
| 144 | ++ |
| 145 | ++ |
| 155 | ++ |
| 156 | ++ |
| 157 | ++ |
| 158 | ++ |
| 160 | ++ |
| 161 | ++ |
| 162 | ++ |
| 163 | ++ |
| 166 | + |
| 167 | ++ |
| 168 | +++ |
| 169 | +++ |
| 170 | + |
| 173 | ++ |
| 178 | + |
| 180 | ++ |
| 182 | ++ |
| 183 | ++ |
| 194 | ++ |
| 195 | +++ |
| 196 | +++ |
| 198 | + |
| 199 | ++ |
| 201 | ++ |
| 202 | + |
| 204 | ++ |
| 205 | ++ |
| 206 | + |
| 207 | ++ |
| 208 | ++ |
| 209 | ++ |
| 210 | ++ |
| 212 | + |
| 213 | + |
| 214 | ++ |
| 215 | ++ |
| 218 | ++ |
| 219 | ++ |
| 220 | ++ |
| 221 | ++ |
| 222 | ++ |
| 223 | ++ |
| 224 | ++ |
| 230 | ++ |
| 233 | +++ |
| 235 | + |
| 239 | ++ |
| 240 | ++ |
| 241 | ++ |
| 242 | + |
| 243 | ++ |
| 244 | ++ |
| 245 | + |
| 246 | ++ |
| 247 | ++ |
| 248 | + |
| 249 | + |
| 250 | +++ |
| 252 | ++ |
| 253 | ++ |
| 254 | ++ |
| 255 | ++ |
| 256 | + |
| 257 | + |
| 259 | + |
| 260 | + |
| 263 | ++ |
| 264 | ++ |
| 268 | + |
| 272 | +++ |
| 273 | ++ |
| 274 | ++ |
| 275 | + |
| 276 | + |
| 277 | + |
| 278 | ++ |
| 279 | +++ |
| 283 | ++ |
| 284 | +++ |
| 285 | ++ |
| 286 | ++ |
| 287 | + |
| 288 | ++ |
| 289 | +++ |
| 290 | + |
| 291 | ++ |
| 292 | ++ |
| 293 | ++ |
| 296 | + |
| 297 | + |
| 298 | +++ |
| 299 | ++ |
| 301 | ++ |
| 304 | + |
| 305 | ++ |
| 306 | ++ |
| 307 | ++ |
| 308 | + |
| 312 | ++ |
| 313 | ++ |
| 314 | + |
| 316 | ++ |
| 317 | + |
| 318 | + |
| 319 | ++ |
| 320 | ++ |
| 321 | ++ |
| 322 | ++ |
| 323 | + |
| 324 | ++ |
| 325 | ++ |
| 328 | ++ |
| 329 | ++ |
| 333 | + |
| 336 | ++ |
| 337 | + |
| 339 | ++ |
| 340 | ++ |
| 341 | + |
| 342 | +++ |
| 343 | +++ |
| 344 | ++ |
| 345 | ++ |
| 346 | +++ |
| 347 | ++ |
| 348 | ++ |
| 349 | ++ |
| 350 | ++ |
| 351 | + |
| 361 | + |
| 366 | ++ |
| 368 | ++ |
| 369 | ++ |
| 370 | ++ |
| 371 | +++ |
| 373 | + |
| 374 | ++ |
| 390 | ++ |
| 391 | ++ |
| 392 | ++ |
| 393 | ++ |
| 395 | ++ |
| 396 | ++ |
| 397 | ++ |
| 398 | ++ |
| 404 | ++ |
| 406 | + |
| 411 | ++ |
| 413 | ++ |
| 415 | + |
| 416 | +++ |
| 417 | +++ |
| 419 | ++ |
| 420 | + |
| 421 | +++ |
| 422 | ++ |
| 423 | ++ |
| 425 | ++ |
| 436 | ++ |
| 437 | ++ |
| 439 | ++ |
| 440 | ++ |
| 441 | ++ |
| 442 | ++ |
| 443 | ++ |
| 446 | ++ |
| 447 | +++ |
| 449 | ++ |
| 450 | ++ |
| 453 | ++ |
| 454 | ++ |
| 455 | ++ |
| 456 | ++ |
| 457 | ++ |
| 458 | ++ |
| 462 | +++ |
| 464 | + |
| 466 | ++ |
| 467 | ++ |
| 469 | +++ |
| 470 | +++ |
| 471 | ++ |
| 472 | + |
| 473 | +++ |
| 475 | +++ |
| 476 | +++ |
| 477 | +++ |
| 479 | ++ |
| 487 | ++ |
| 488 | + |
| 489 | ++ |
| 490 | ++ |
| 491 | ++ |
| 492 | ++ |
| 496 | ++ |
| 497 | ++ |
| 498 | ++ |
| 504 | ++ |
| 505 | ++ |
| 506 | ++ |
| 507 | ++ |
| 508 | ++ |
| 509 | ++ |
| 510 | ++ |
| 511 | ++ |
| 512 | +++ |
| 513 | ++ |
| 514 | ++ |
| 515 | ++ |
| 516 | +++ |
| 517 | ++ |
| 518 | ++ |
| 519 | ++ |
| 520 | +++ |
| 521 | ++ |
| 522 | ++ |
| 523 | +++ |
| 524 | ++ |
| 525 | ++ |
| 526 | ++ |
| 527 | ++ |
| 528 | ++ |
| 529 | ++ |
| 530 | ++ |
| 532 | +++ |
| 533 | ++ |
| 534 | + |
| 535 | ++ |
| 537 | ++ |
| 538 | ++ |
| 541 | ++ |
| 543 | ++ |
| 544 | +++ |
| 550 | ++ |
| 551 | ++ |
| 552 | ++ |
| 553 | ++ |
| 554 | ++ |
| 555 | +++ |
| 556 | ++ |
| 557 | ++ |
| 558 | ++ |
| 559 | ++ |
| 563 | ++ |
| 566 | + |
| 567 | +++ |
| 569 | ++ |
| 570 | +++ |
| 571 | ++ |
| 572 | ++ |
| 573 | ++ |
| 574 | ++ |
| 575 | ++ |
| 584 | ++ |
| 585 | +++ |
| 587 | ++ |
| 588 | ++ |
| 589 | ++ |
| 590 | ++ |
| 591 | +++ |
| 592 | ++ |
| 593 | + |
| 594 | ++ |
| 601 | ++ |
| 602 | ++ |
| 603 | ++ |
| 604 | ++ |
| 605 | ++ |
| 606 | ++ |
| 607 | ++ |
| 741 | ++ |
| 756 | ++ |
| 757 | ++ |
| 976 | ++ |
| 977 | ++ |
| 980 | ++ |
| 995 | ++ |

Key
+ IC₅₀ >10 uM
++ IC₅₀ 1-10 uM
+++ IC₅₀ <1 uM

Compounds PFP-001 to PFP-864 (below) can be prepared by the schemes set forth in Schemes 1-50 and by the general procedures described herein.

| ID | MOLSTRUCTURE |
|---|---|
| PFP-00001 | 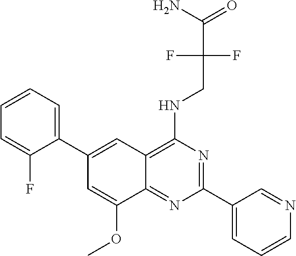 [SMILES: COc1cc(-c2ccccc2F)cc2c(NCC(F)(F)C(N)=O)nc(-c3cccnc3)nc12] |
| PFP-00002 | 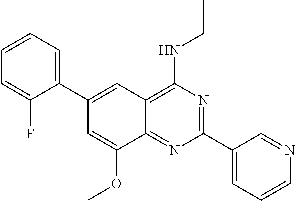 [SMILES: CCNc1nc(-c2cccnc2)nc2c(OC)cc(-c3ccccc3F)cc12] |
| PFP-00003 | 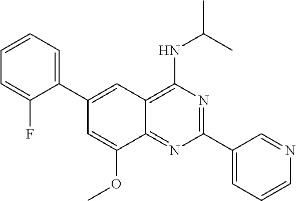 [SMILES: COc1cc(-c2ccccc2F)cc2c(NC(C)C)nc(-c3cccnc3)nc12] |
| PFP-00004 | 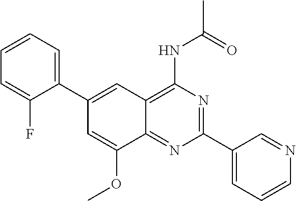 [SMILES: COc1cc(-c2ccccc2F)cc2c(NC(C)=O)nc(-c3cccnc3)nc12] |
| PFP-00005 | 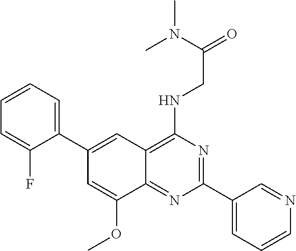 [SMILES: COc1cc(-c2ccccc2F)cc2c(NCC(=O)N(C)C)nc(-c3cccnc3)nc12] |
| PFP-00006 | 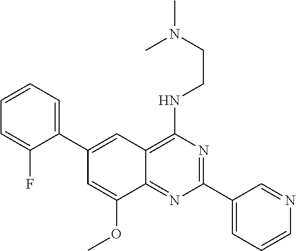 [SMILES: COc1cc(-c2ccccc2F)cc2c(NCCN(C)C)nc(-c3cccnc3)nc12] |
| PFP-00007 | 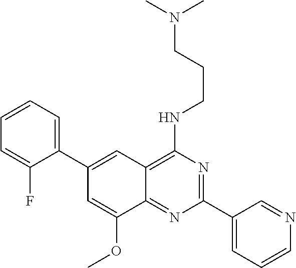 [SMILES: COc1cc(-c2ccccc2F)cc2c(NCCCN(C)C)nc(-c3cccnc3)nc12] |
| PFP-00008 | 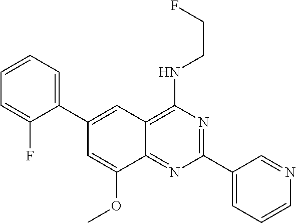 [SMILES: COc1cc(-c2ccccc2F)cc2c(NCCF)nc(-c3cccnc3)nc12] |
| PFP-00009 | 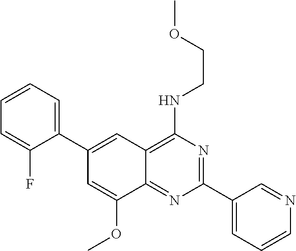 [SMILES: COCCNc1nc(-c2cccnc2)nc2c(OC)cc(-c3ccccc3F)cc12] |
| PFP-00010 | 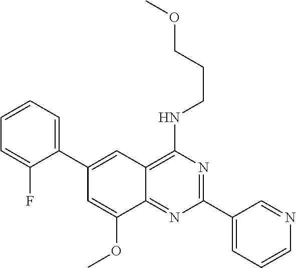 [SMILES: COCCCNc1nc(-c2cccnc2)nc2c(OC)cc(-c3ccccc3F)cc12] |
| PFP-00011 | 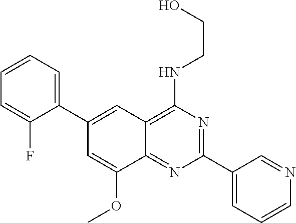 [SMILES: COc1cc(-c2ccccc2F)cc2c(NCCO)nc(-c3cccnc3)nc12] |
| PFP-00012 | 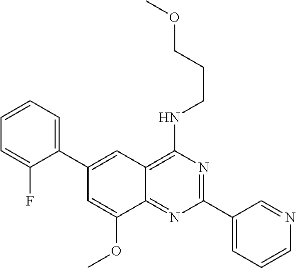 [SMILES: COCCCNc1nc(-c2cccnc2)nc2c(OC)cc(-c3ccccc3F)cc12] |
| PFP-00013 | 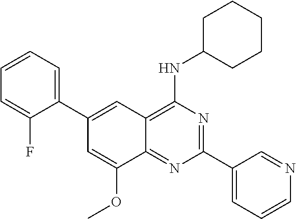 [SMILES: COc1cc(-c2ccccc2F)cc2c(NC3CCCCC3)nc(-c3cccnc3)nc12] |
| PFP-00014 | 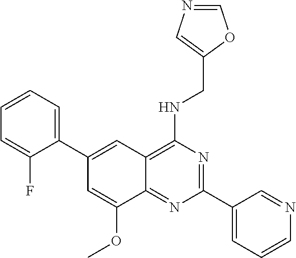 [SMILES: COc1cc(-c2ccccc2F)cc2c(NCc3cnco3)nc(-c3cccnc3)nc12] |
| PFP-00015 | 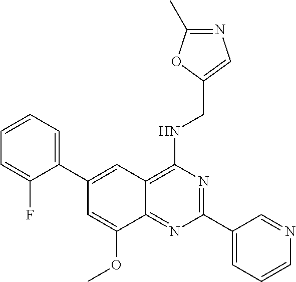 [SMILES: COc1cc(-c2ccccc2F)cc2c(NCc3cnc(C)o3)nc(-c3cccnc3)nc12] |
| PFP-00016 | 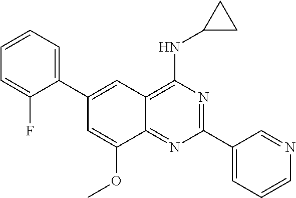 [SMILES: COc1cc(-c2ccccc2F)cc2c(NC3CC3)nc(-c3cccnc3)nc12] |
| PFP-00017 | 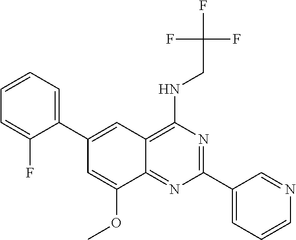 [SMILES: COc1cc(-c2ccccc2F)cc2c(NCC(F)(F)F)nc(-c3cccnc3)nc12] |
| PFP-00018 | 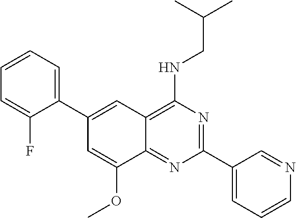 [SMILES: COc1cc(-c2ccccc2F)cc2c(NCC(C)C)nc(-c3cccnc3)nc12] |
| PFP-00019 | 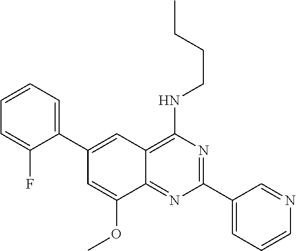 [SMILES: CCCCNc1nc(-c2cccnc2)nc2c(OC)cc(-c3ccccc3F)cc12] |
| PFP-00020 | 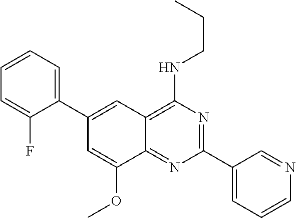 [SMILES: CCCNc1nc(-c2cccnc2)nc2c(OC)cc(-c3ccccc3F)cc12] |
| PFP-00021 | 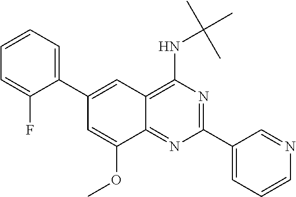 [SMILES: COc1cc(-c2ccccc2F)cc2c(NC(C)(C)C)nc(-c3cccnc3)nc12] |
| PFP-00022 | 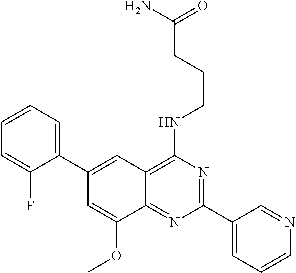 [SMILES: COc1cc(-c2ccccc2F)cc2c(NCCCC(N)=O)nc(-c3cccnc3)nc12] |
| PFP-00023 | 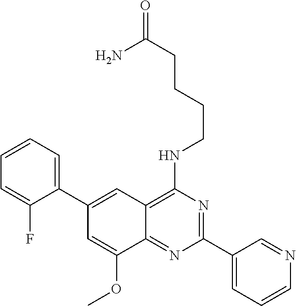 [SMILES: COc1cc(-c2ccccc2F)cc2c(NCCCCC(N)=O)nc(-c3cccnc3)nc12] |
| PFP-00024 | 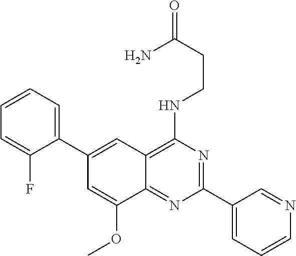 [SMILES: COc1cc(-c2ccccc2F)cc2c(NCCC(N)=O)nc(-c3cccnc3)nc12] |
| PFP-00025 | 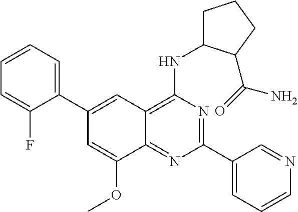 [SMILES: COc1cc(-c2ccccc2F)cc2c(NC3CCCC3C(N)=O)nc(-c3cccnc3)nc12] |
| PFP-00026 | 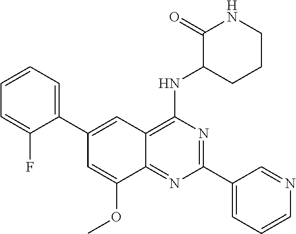 [SMILES: COc1cc(-c2ccccc2F)cc2c(NC3CCCNC3=O)nc(-c3cccnc3)nc12] |
| PFP-00027 | 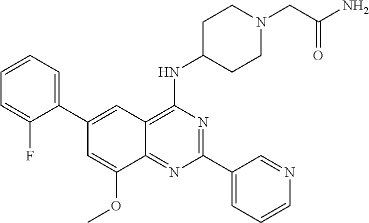 [SMILES: COc1cc(-c2ccccc2F)cc2c(NC3CCN(CC(N)=O)CC3)nc(-c3cccnc3)nc12] |
| PFP-00028 | 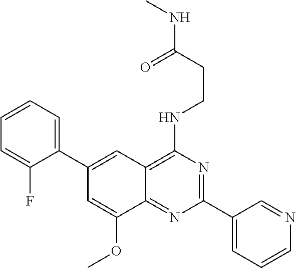 [SMILES: CNC(=O)CCNc1nc(-c2cccnc2)nc2c(OC)cc(-c3ccccc3F)cc12] |
| PFP-00029 | 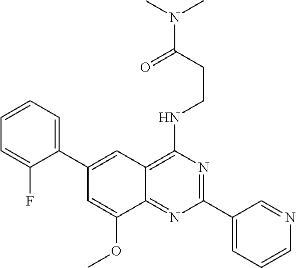 [SMILES: COc1cc(-c2ccccc2F)cc2c(NCCC(=O)N(C)C)nc(-c3cccnc3)nc12] |
| PFP-00030 | 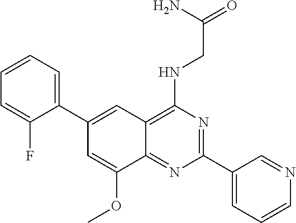 [SMILES: COc1cc(-c2ccccc2F)cc2c(NCC(N)=O)nc(-c3cccnc3)nc12] |
| PFP-00031 | 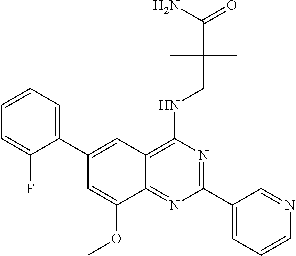 [SMILES: COc1cc(-c2ccccc2F)cc2c(NCC(C)(C)C(N)=O)nc(-c3cccnc3)nc12] |
| PFP-00032 | 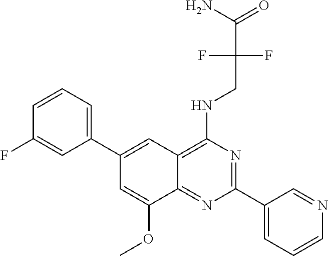 [SMILES: COc1cc(-c2cccc(F)c2)cc2c(NCC(F)(F)C(N)=O)nc(-c3cccnc3)nc12] |
| PFP-00033 | 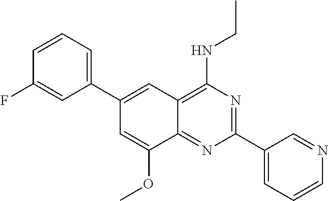 [SMILES: CCNc1nc(-c2cccnc2)nc2c(OC)cc(-c3cccc(F)c3)cc12] |
| PFP-00034 | 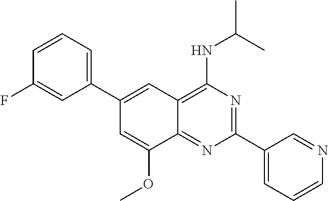 [SMILES: COc1cc(-c2cccc(F)c2)cc2c(NC(C)C)nc(-c3cccnc3)nc12] |
| PFP-00035 | 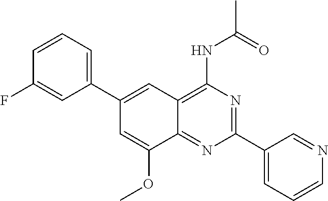 [SMILES: COc1cc(-c2cccc(F)c2)cc2c(NC(C)=O)nc(-c3cccnc3)nc12] |
| PFP-00036 | 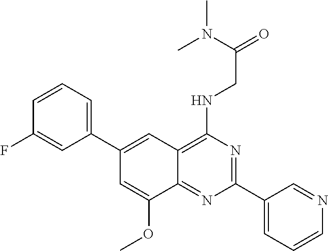 [SMILES: COc1cc(-c2cccc(F)c2)cc2c(NCC(=O)N(C)C)nc(-c3cccnc3)nc12] |
| PFP-00037 | 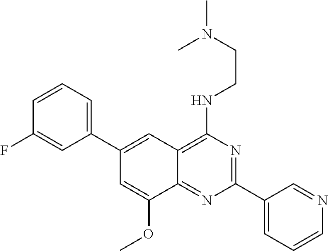 [SMILES: COc1cc(-c2cccc(F)c2)cc2c(NCCN(C)C)nc(-c3cccnc3)nc12] |
| PFP-00038 | 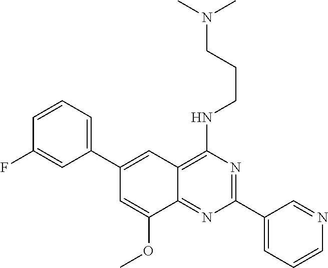 [SMILES: COc1cc(-c2cccc(F)c2)cc2c(NCCCN(C)C)nc(-c3cccnc3)nc12] |
| PFP-00039 | 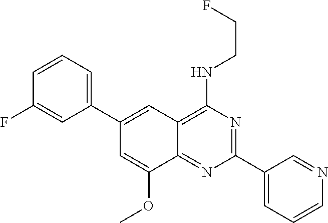 [SMILES: COc1cc(-c2cccc(F)c2)cc2c(NCCF)nc(-c3cccnc3)nc12] |
| PFP-00040 | 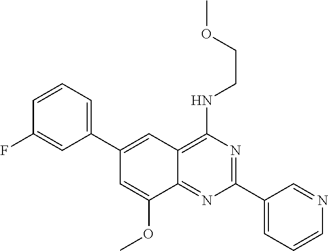 [SMILES: COCCNc1nc(-c2cccnc2)nc2c(OC)cc(-c3cccc(F)c3)cc12] |
| PFP-00041 | 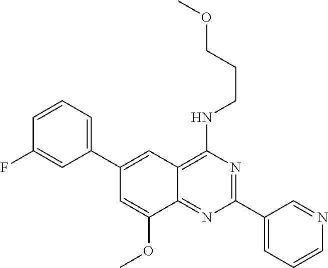 [SMILES: COCCCNc1nc(-c2cccnc2)nc2c(OC)cc(-c3cccc(F)c3)cc12] |
| PFP-00042 | 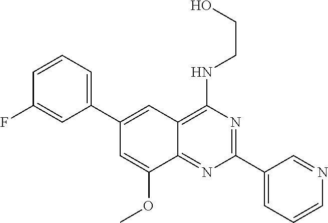 [SMILES: COc1cc(-c2cccc(F)c2)cc2c(NCCO)nc(-c3cccnc3)nc12] |
| PFP-00043 | 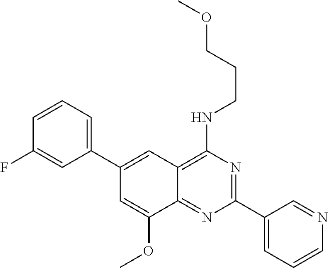 [SMILES: COCCCNc1nc(-c2cccnc2)nc2c(OC)cc(-c3cccc(F)c3)cc12] |
| PFP-00044 | 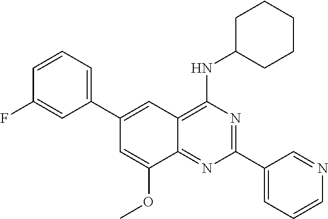 [SMILES: COc1cc(-c2cccc(F)c2)cc2c(NC3CCCCC3)nc(-c3cccnc3)nc12] |
| PFP-00045 | 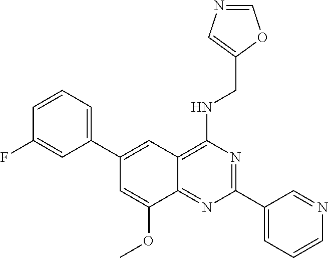 [SMILES: COc1cc(-c2cccc(F)c2)cc2c(NCc3cnco3)nc(-c3cccnc3)nc12] |
| PFP-00046 | 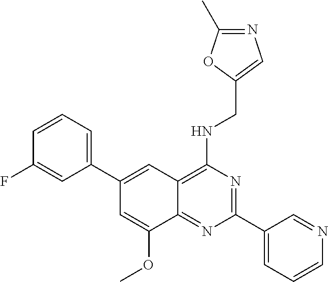 [SMILES: COc1cc(-c2cccc(F)c2)cc2c(NCc3cnc(C)o3)nc(-c3cccnc3)nc12] |
| PFP-00047 | 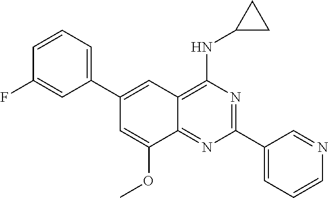 [SMILES: COc1cc(-c2cccc(F)c2)cc2c(NC3CC3)nc(-c3cccnc3)nc12] |
| PFP-00048 | 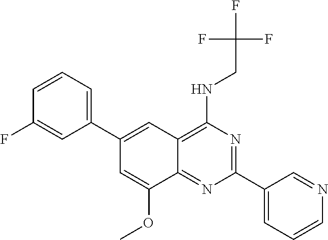 [SMILES: COc1cc(-c2cccc(F)c2)cc2c(NCC(F)(F)F)nc(-c3cccnc3)nc12] |
| PFP-00049 | 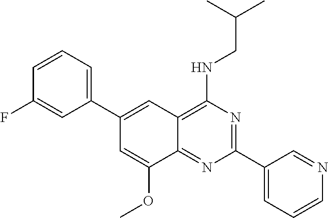 [SMILES: COc1cc(-c2cccc(F)c2)cc2c(NCC(C)C)nc(-c3cccnc3)nc12] |
| PFP-00050 | 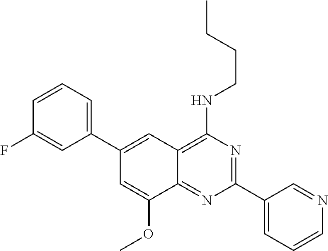 [SMILES: CCCCNc1nc(-c2cccnc2)nc2c(OC)cc(-c3cccc(F)c3)cc12] |
| PFP-00051 | 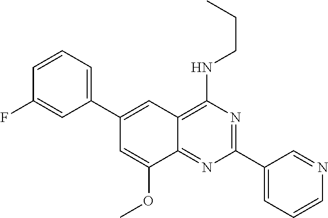 [SMILES: CCCNc1nc(-c2cccnc2)nc2c(OC)cc(-c3cccc(F)c3)cc12] |
| PFP-00052 | 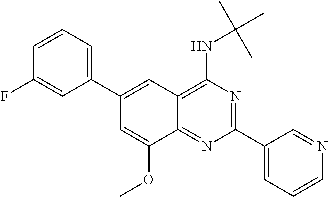 [SMILES: COc1cc(-c2cccc(F)c2)cc2c(NC(C)(C)C)nc(-c3cccnc3)nc12] |
| PFP-00053 | 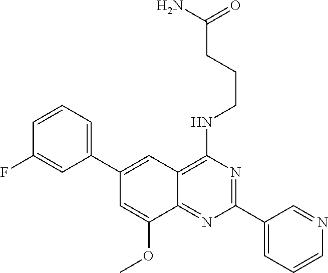 [SMILES: COc1cc(-c2cccc(F)c2)cc2c(NCCCC(N)=O)nc(-c3cccnc3)nc12] |
| PFP-00054 | 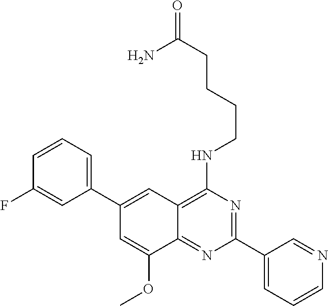 [SMILES: COc1cc(-c2cccc(F)c2)cc2c(NCCCCC(N)=O)nc(-c3cccnc3)nc12] |
| PFP-00055 | 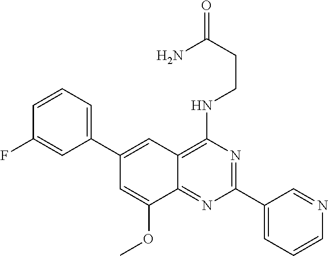 [SMILES: COc1cc(-c2cccc(F)c2)cc2c(NCCC(N)=O)nc(-c3cccnc3)nc12] |
| PFP-00056 | 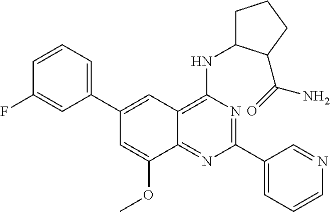 [SMILES: COc1cc(-c2cccc(F)c2)cc2c(NC3CCCC3C(N)=O)nc(-c3cccnc3)nc12] |
| PFP-00057 | 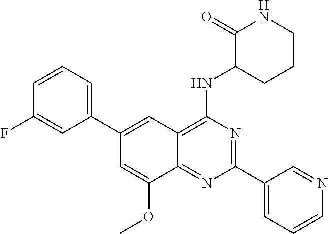 [SMILES: COc1cc(-c2cccc(F)c2)cc2c(NC3CCCNC3=O)nc(-c3cccnc3)nc12] |
| PFP-00058 | 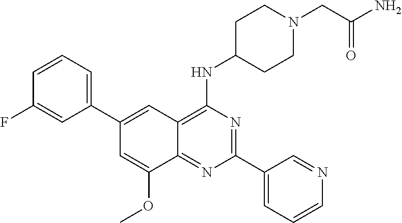 [SMILES: COc1cc(-c2cccc(F)c2)cc2c(NC3CCN(CC(N)=O)CC3)nc(-c3cccnc3)nc12] |
| PFP-00059 | 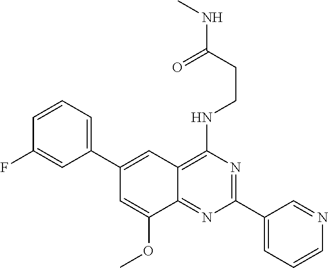 [SMILES: CNC(=O)CCNc1nc(-c2cccnc2)nc2c(OC)cc(-c3cccc(F)c3)cc12] |
| PFP-00060 | 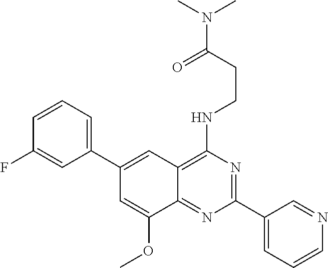 [SMILES: COc1cc(-c2cccc(F)c2)cc2c(NCCC(=O)N(C)C)nc(-c3cccnc3)nc12] |
| PFP-00061 | 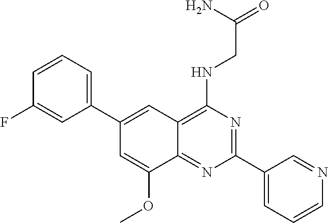 [SMILES: COc1cc(-c2cccc(F)c2)cc2c(NCC(N)=O)nc(-c3cccnc3)nc12] |
| PFP-00062 | 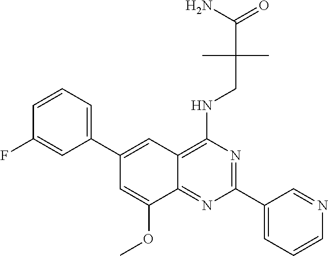 [SMILES: COc1cc(-c2cccc(F)c2)cc2c(NCC(C)(C)C(N)=O)nc(-c3cccnc3)nc12] |
| PFP-00063 | 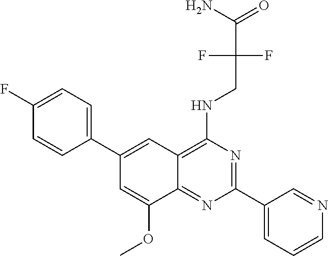 [SMILES: COc1cc(-c2ccc(F)cc2)cc2c(NCC(F)(F)C(N)=O)nc(-c3cccnc3)nc12] |
| PFP-00064 | 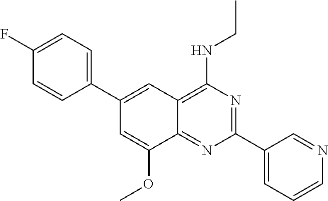 [SMILES: CCNc1nc(-c2cccnc2)nc2c(OC)cc(-c3ccc(F)cc3)cc12] |
| PFP-00065 | 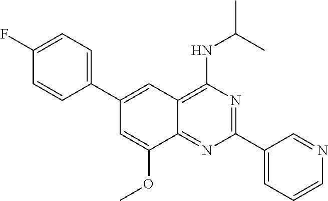 [SMILES: COc1cc(-c2ccc(F)cc2)cc2c(NC(C)C)nc(-c3cccnc3)nc12] |
| PFP-00066 | 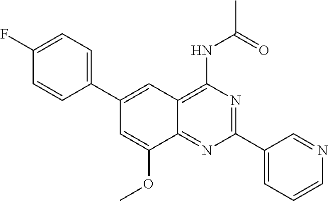 [SMILES: COc1cc(-c2ccc(F)cc2)cc2c(NC(C)=O)nc(-c3cccnc3)nc12] |
| PFP-00067 | 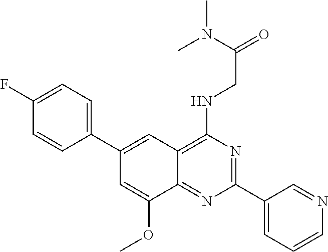 [SMILES: COc1cc(-c2ccc(F)cc2)cc2c(NCC(=O)N(C)C)nc(-c3cccnc3)nc12] |
| PFP-00068 | 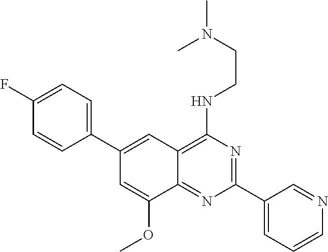 [SMILES: COc1cc(-c2ccc(F)cc2)cc2c(NCCN(C)C)nc(-c3cccnc3)nc12] |
| PFP-00069 | 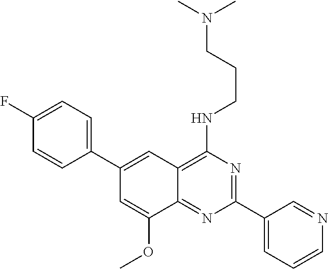 [SMILES: COc1cc(-c2ccc(F)cc2)cc2c(NCCCN(C)C)nc(-c3cccnc3)nc12] |
| PFP-00070 | 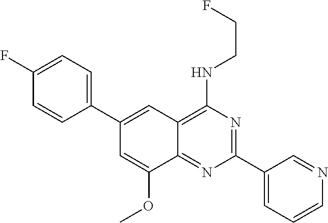 [SMILES: COc1cc(-c2ccc(F)cc2)cc2c(NCCF)nc(-c3cccnc3)nc12] |
| PFP-00071 | 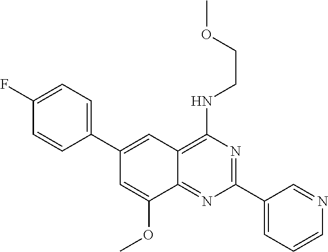 [SMILES: COCCNc1nc(-c2cccnc2)nc2c(OC)cc(-c3ccc(F)cc3)cc12] |
| PFP-00072 | 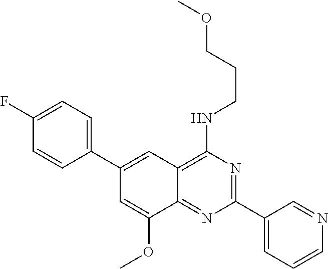 [SMILES: COCCCNc1nc(-c2cccnc2)nc2c(OC)cc(-c3ccc(F)cc3)cc12] |
| PFP-00073 | 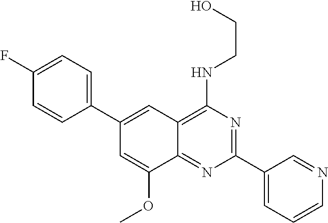 [SMILES: COc1cc(-c2ccc(F)cc2)cc2c(NCCO)nc(-c3cccnc3)nc12] |
| PFP-00074 | 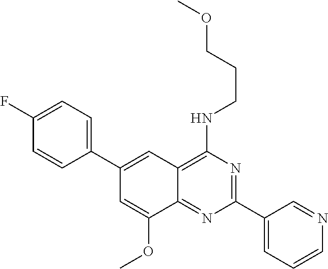 [SMILES: COCCCNc1nc(-c2cccnc2)nc2c(OC)cc(-c3ccc(F)cc3)cc12] |
| PFP-00075 | 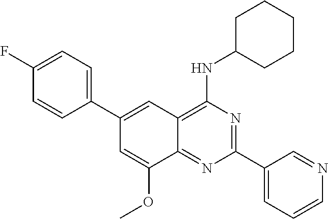 [SMILES: COc1cc(-c2ccc(F)cc2)cc2c(NC3CCCCC3)nc(-c3cccnc3)nc12] |
| PFP-00076 | 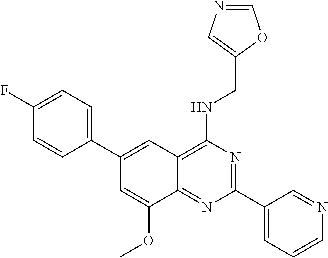 [SMILES: COc1cc(-c2ccc(F)cc2)cc2c(NCc3cnco3)nc(-c3cccnc3)nc12] |
| PFP-00077 | 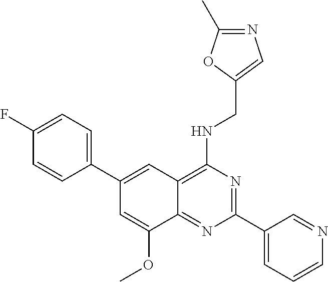 [SMILES: COc1cc(-c2ccc(F)cc2)cc2c(NCc3cnc(C)o3)nc(-c3cccnc3)nc12] |
| PFP-00078 | 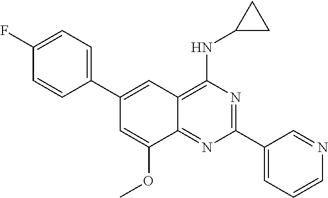 [SMILES: COc1cc(-c2ccc(F)cc2)cc2c(NC3CC3)nc(-c3cccnc3)nc12] |
| PFP-00079 | 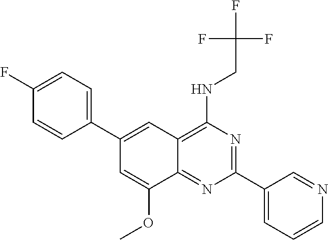 [SMILES: COc1cc(-c2ccc(F)cc2)cc2c(NCC(F)(F)F)nc(-c3cccnc3)nc12] |
| PFP-00080 | 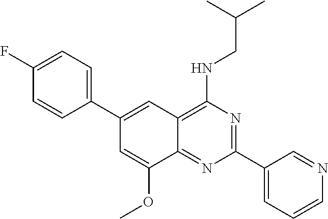 [SMILES: COc1cc(-c2ccc(F)cc2)cc2c(NCC(C)C)nc(-c3cccnc3)nc12] |
| PFP-00081 | 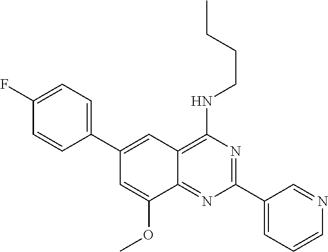 [SMILES: CCCCNc1nc(-c2cccnc2)nc2c(OC)cc(-c3ccc(F)cc3)cc12] |
| PFP-00082 | 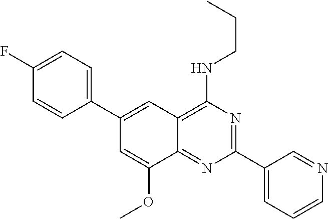 [SMILES: CCCNc1nc(-c2cccnc2)nc2c(OC)cc(-c3ccc(F)cc3)cc12] |
| PFP-00083 | 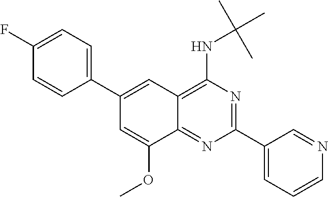 [SMILES: COc1cc(-c2ccc(F)cc2)cc2c(NC(C)(C)C)nc(-c3cccnc3)nc12] |
| PFP-00084 | 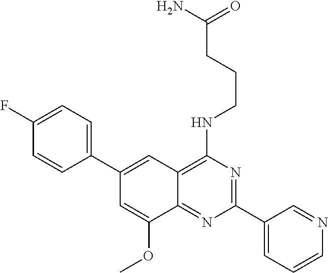 [SMILES: COc1cc(-c2ccc(F)cc2)cc2c(NCCCC(N)=O)nc(-c3cccnc3)nc12] |
| PFP-00085 | 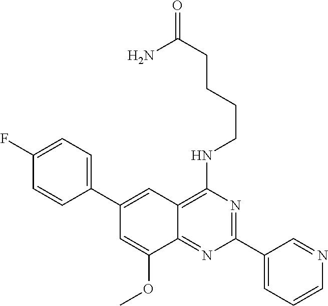 [SMILES: COc1cc(-c2ccc(F)cc2)cc2c(NCCCCC(N)=O)nc(-c3cccnc3)nc12] |
| PFP-00086 | 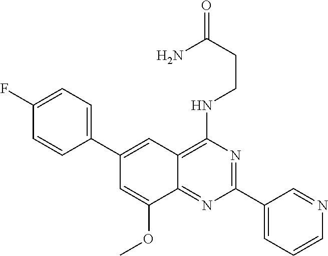 [SMILES: COc1cc(-c2ccc(F)cc2)cc2c(NCCC(N)=O)nc(-c3cccnc3)nc12] |
| PFP-00087 | 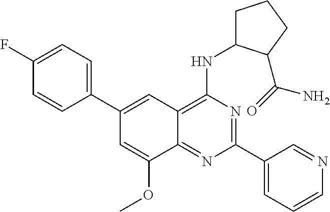 [SMILES: COc1cc(-c2ccc(F)cc2)cc2c(NC3CCCC3C(N)=O)nc(-c3cccnc3)nc12] |
| PFP-00088 | 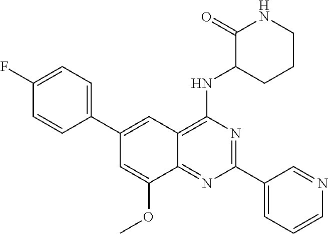 [SMILES: COc1cc(-c2ccc(F)cc2)cc2c(NC3CCCNC3=O)nc(-c3cccnc3)nc12] |
| PFP-00089 | 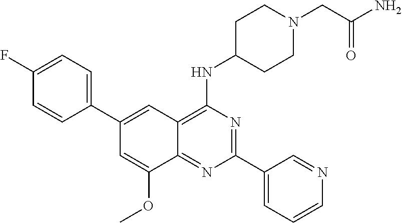 [SMILES: COc1cc(-c2ccc(F)cc2)cc2c(NC3CCN(CC(N)=O)CC3)nc(-c3cccnc3)nc12] |
| PFP-00090 | 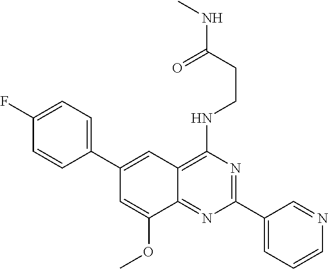 [SMILES: CNC(=O)CCNc1nc(-c2cccnc2)nc2c(OC)cc(-c3ccc(F)cc3)cc12] |
| PFP-00091 | 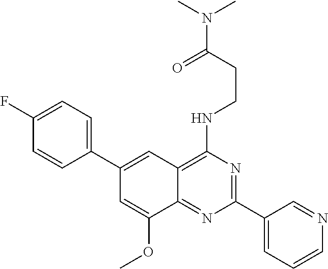 [SMILES: COc1cc(-c2ccc(F)cc2)cc2c(NCCC(=O)N(C)C)nc(-c3cccnc3)nc12] |
| PFP-00092 | 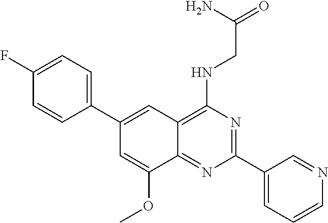 [SMILES: COc1cc(-c2ccc(F)cc2)cc2c(NCC(N)=O)nc(-c3cccnc3)nc12] |
| PFP-00093 | 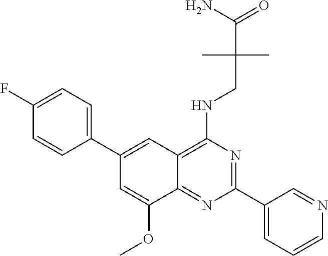 [SMILES: COc1cc(-c2ccc(F)cc2)cc2c(NCC(C)(C)C(N)=O)nc(-c3cccnc3)nc12] |
| PFP-00094 | 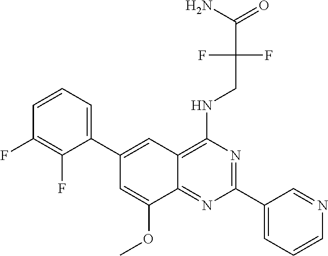 [SMILES: COc1cc(-c2cccc(F)c2F)cc2c(NCC(F)(F)C(N)=O)nc(-c3cccnc3)nc12] |
| PFP-00095 | 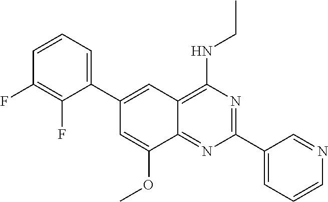 [SMILES: CCNc1nc(-c2cccnc2)nc2c(OC)cc(-c3cccc(F)c3F)cc12] |
| PFP-00096 | 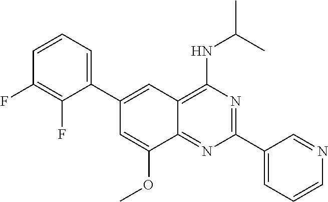 [SMILES: COc1cc(-c2cccc(F)c2F)cc2c(NC(C)C)nc(-c3cccnc3)nc12] |
| PFP-00097 | 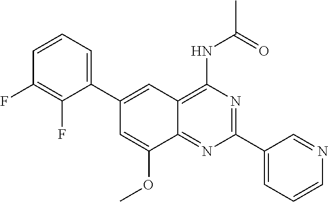 [SMILES: COc1cc(-c2cccc(F)c2F)cc2c(NC(C)=O)nc(-c3cccnc3)nc12] |
| PFP-00098 | 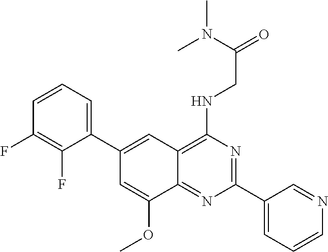 [SMILES: COc1cc(-c2cccc(F)c2F)cc2c(NCC(=O)N(C)C)nc(-c3cccnc3)nc12] |
| PFP-00099 | 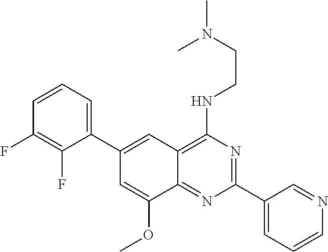 [SMILES: COc1cc(-c2cccc(F)c2F)cc2c(NCCN(C)C)nc(-c3cccnc3)nc12] |
| PFP-00100 | 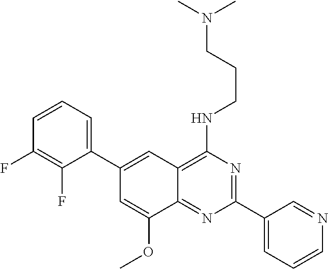 [SMILES: COc1cc(-c2cccc(F)c2F)cc2c(NCCCN(C)C)nc(-c3cccnc3)nc12] |
| PFP-00101 | 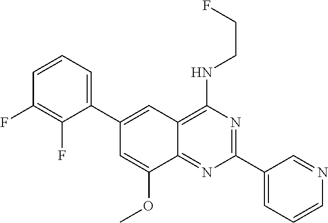 [SMILES: COc1cc(-c2cccc(F)c2F)cc2c(NCCF)nc(-c3cccnc3)nc12] |
| PFP-00102 | 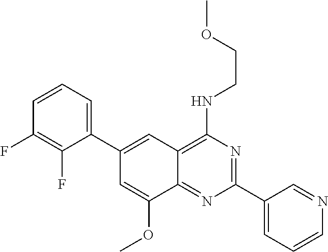 [SMILES: COCCNc1nc(-c2cccnc2)nc2c(OC)cc(-c3cccc(F)c3F)cc12] |
| PFP-00103 | 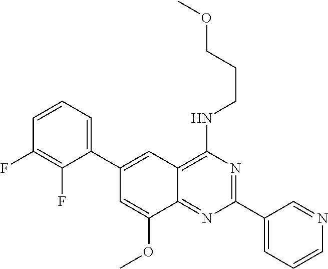 [SMILES: COCCCNc1nc(-c2cccnc2)nc2c(OC)cc(-c3cccc(F)c3F)cc12] |
| PFP-00104 | 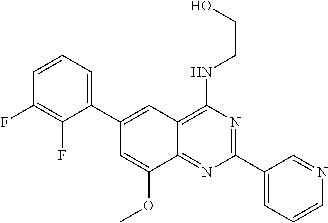 [SMILES: COc1cc(-c2cccc(F)c2F)cc2c(NCCO)nc(-c3cccnc3)nc12] |
| PFP-00105 | 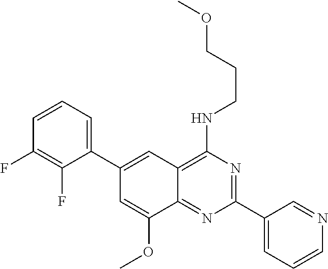 [SMILES: COCCCNc1nc(-c2cccnc2)nc2c(OC)cc(-c3cccc(F)c3F)cc12] |
| PFP-00106 | 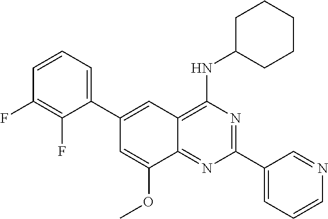 [SMILES: COc1cc(-c2cccc(F)c2F)cc2c(NC3CCCCC3)nc(-c3cccnc3)nc12] |
| PFP-00107 | 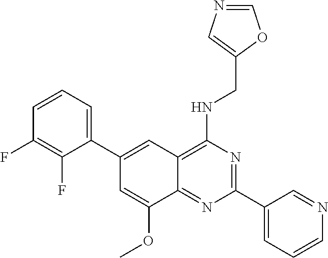 [SMILES: COc1cc(-c2cccc(F)c2F)cc2c(NCc3cnco3)nc(-c3cccnc3)nc12] |
| PFP-00108 | 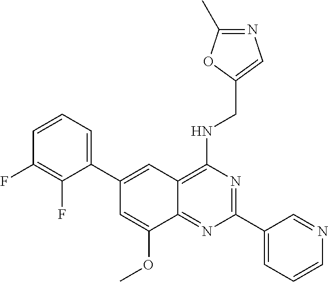 [SMILES: COc1cc(-c2cccc(F)c2F)cc2c(NCc3cnc(C)o3)nc(-c3cccnc3)nc12] |
| PFP-00109 | 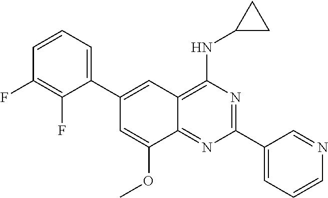 [SMILES: COc1cc(-c2cccc(F)c2F)cc2c(NC3CC3)nc(-c3cccnc3)nc12] |
| PFP-00110 | 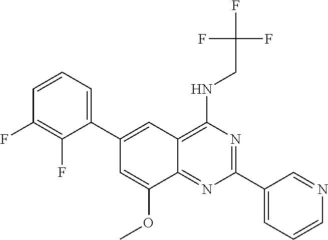 [SMILES: COc1cc(-c2cccc(F)c2F)cc2c(NCC(F)(F)F)nc(-c3cccnc3)nc12] |
| PFP-00111 | 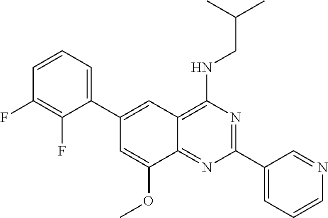 [SMILES: COc1cc(-c2cccc(F)c2F)cc2c(NCC(C)C)nc(-c3cccnc3)nc12] |
| PFP-00112 | 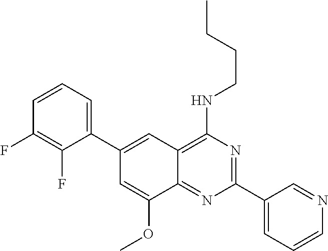 [SMILES: CCCCNc1nc(-c2cccnc2)nc2c(OC)cc(-c3cccc(F)c3F)cc12] |
| PFP-00113 | 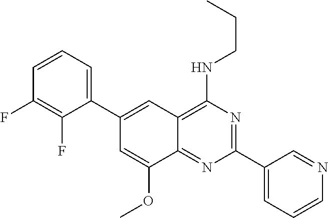 [SMILES: CCCNc1nc(-c2cccnc2)nc2c(OC)cc(-c3cccc(F)c3F)cc12] |
| PFP-00114 | 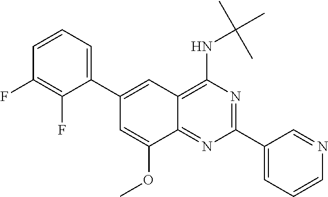 [SMILES: COc1cc(-c2cccc(F)c2F)cc2c(NC(C)(C)C)nc(-c3cccnc3)nc12] |
| PFP-00115 | 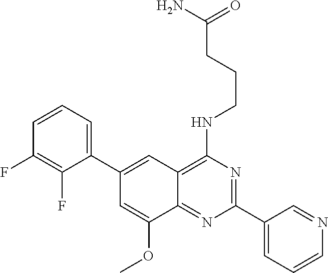 [SMILES: COc1cc(-c2cccc(F)c2F)cc2c(NCCCC(N)=O)nc(-c3cccnc3)nc12] |
| PFP-00116 | 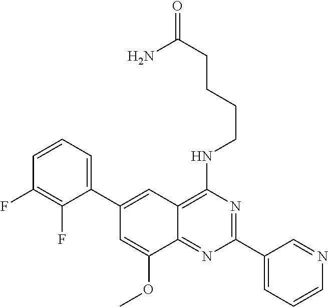 [SMILES: COc1cc(-c2cccc(F)c2F)cc2c(NCCCCC(N)=O)nc(-c3cccnc3)nc12] |
| PFP-00117 | 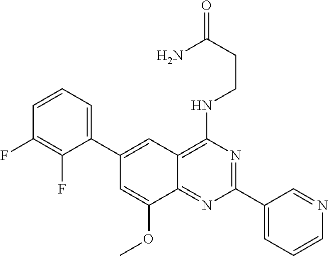 [SMILES: COc1cc(-c2cccc(F)c2F)cc2c(NCCC(N)=O)nc(-c3cccnc3)nc12] |
| PFP-00118 | 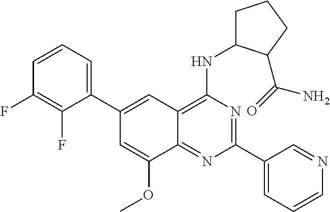 [SMILES: COc1cc(-c2cccc(F)c2F)cc2c(NC3CCCC3C(N)=O)nc(-c3cccnc3)nc12] |
| PFP-00119 | 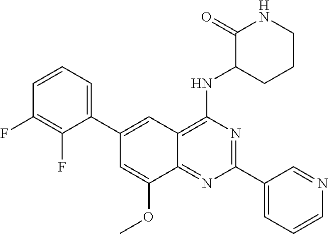 [SMILES: COc1cc(-c2cccc(F)c2F)cc2c(NC3CCCNC3=O)nc(-c3cccnc3)nc12] |
| PFP-00120 | 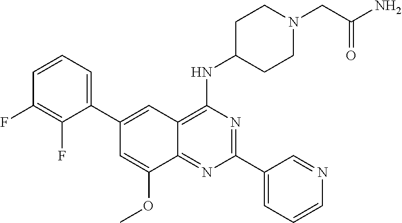 [SMILES: COc1cc(-c2cccc(F)c2F)cc2c(NC3CCN(CC(N)=O)CC3)nc(-c3cccnc3)nc12] |
| PFP-00121 | 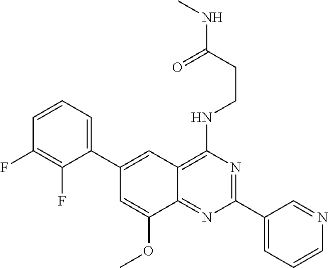 [SMILES: CNC(=O)CCNc1nc(-c2cccnc2)nc2c(OC)cc(-c3cccc(F)c3F)cc12] |
| PFP-00122 | 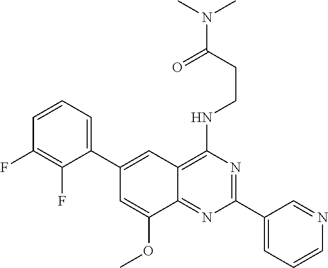 [SMILES: COc1cc(-c2cccc(F)c2F)cc2c(NCCC(=O)N(C)C)nc(-c3cccnc3)nc12] |
| PFP-00123 | 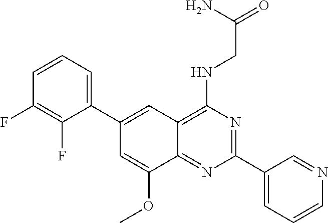 [SMILES: COc1cc(-c2cccc(F)c2F)cc2c(NCC(N)=O)nc(-c3cccnc3)nc12] |
| PFP-00124 | 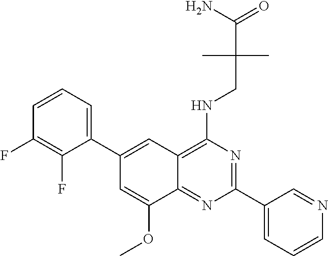 [SMILES: COc1cc(-c2cccc(F)c2F)cc2c(NCC(C)(C)C(N)=O)nc(-c3cccnc3)nc12] |
| PFP-00125 | 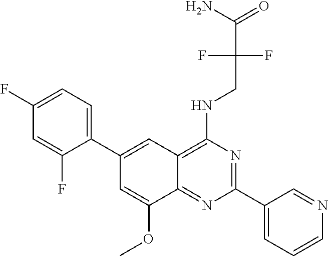 [SMILES: COc1cc(-c2ccc(F)cc2F)cc2c(NCC(F)(F)C(N)=O)nc(-c3cccnc3)nc12] |
| PFP-00126 | 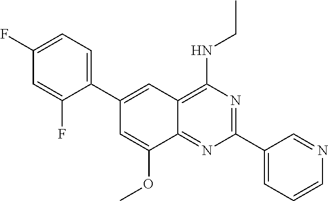 [SMILES: CCNc1nc(-c2cccnc2)nc2c(OC)cc(-c3ccc(F)cc3F)cc12] |
| PFP-00127 | 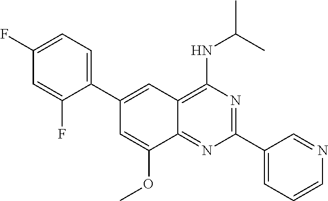 [SMILES: COc1cc(-c2ccc(F)cc2F)cc2c(NC(C)C)nc(-c3cccnc3)nc12] |
| PFP-00128 | 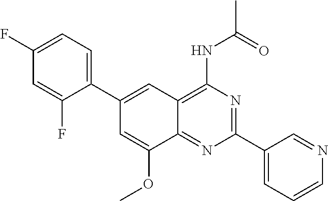 [SMILES: COc1cc(-c2ccc(F)cc2F)cc2c(NC(C)=O)nc(-c3cccnc3)nc12] |
| PFP-00129 | 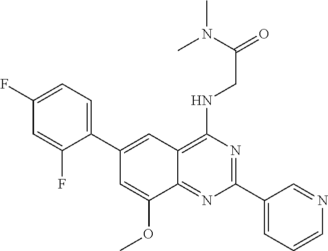 [SMILES: COc1cc(-c2ccc(F)cc2F)cc2c(NCC(=O)N(C)C)nc(-c3cccnc3)nc12] |
| PFP-00130 | 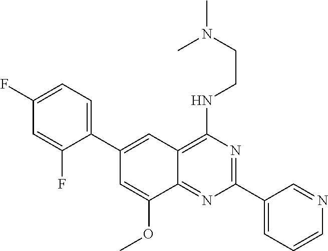 [SMILES: COc1cc(-c2ccc(F)cc2F)cc2c(NCCN(C)C)nc(-c3cccnc3)nc12] |
| PFP-00131 | 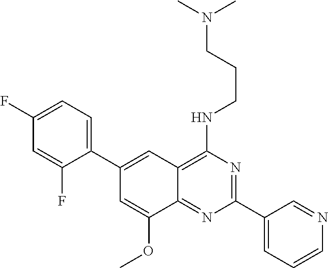 [SMILES: COc1cc(-c2ccc(F)cc2F)cc2c(NCCCN(C)C)nc(-c3cccnc3)nc12] |
| PFP-00132 | 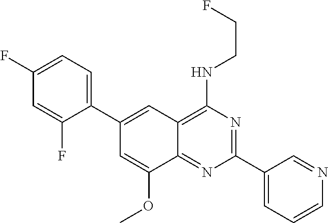 [SMILES: COc1cc(-c2ccc(F)cc2F)cc2c(NCCF)nc(-c3cccnc3)nc12] |
| PFP-00133 | 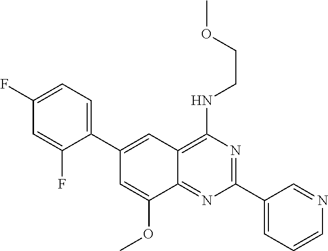 [SMILES: COCCNc1nc(-c2cccnc2)nc2c(OC)cc(-c3ccc(F)cc3F)cc12] |
| PFP-00134 | 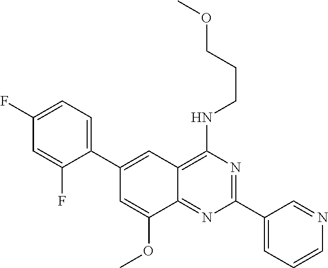 [SMILES: COCCCNc1nc(-c2cccnc2)nc2c(OC)cc(-c3ccc(F)cc3F)cc12] |
| PFP-00135 | 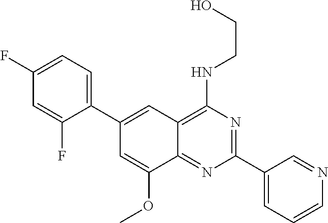 [SMILES: COc1cc(-c2ccc(F)cc2F)cc2c(NCCO)nc(-c3cccnc3)nc12] |
| PFP-00136 | 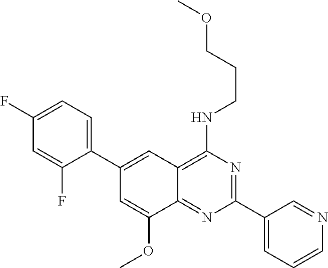 [SMILES: COCCCNc1nc(-c2cccnc2)nc2c(OC)cc(-c3ccc(F)cc3F)cc12] |
| PFP-00137 | 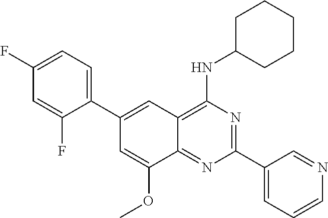 [SMILES: COc1cc(-c2ccc(F)cc2F)cc2c(NC3CCCCC3)nc(-c3cccnc3)nc12] |
| PFP-00138 | 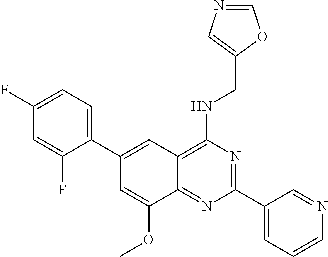 [SMILES: COc1cc(-c2ccc(F)cc2F)cc2c(NCc3cnco3)nc(-c3cccnc3)nc12] |
| PFP-00139 | 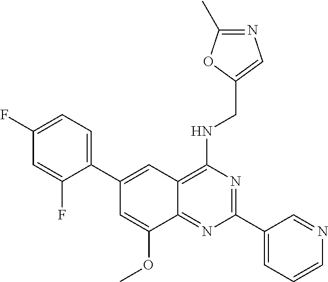 [SMILES: COc1cc(-c2ccc(F)cc2F)cc2c(NCc3cnc(C)o3)nc(-c3cccnc3)nc12] |
| PFP-00140 | 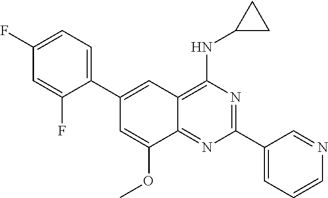 [SMILES: COc1cc(-c2ccc(F)cc2F)cc2c(NC3CC3)nc(-c3cccnc3)nc12] |
| PFP-00141 | 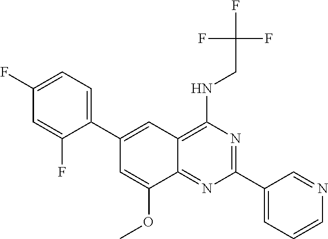 [SMILES: COc1cc(-c2ccc(F)cc2F)cc2c(NCC(F)(F)F)nc(-c3cccnc3)nc12] |
| PFP-00142 | 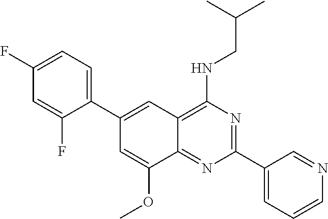 [SMILES: COc1cc(-c2ccc(F)cc2F)cc2c(NCC(C)C)nc(-c3cccnc3)nc12] |
| PFP-00143 | 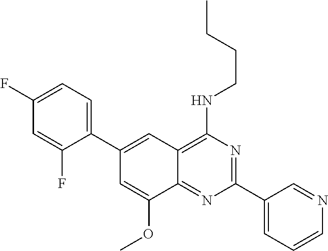 [SMILES: CCCCNc1nc(-c2cccnc2)nc2c(OC)cc(-c3ccc(F)cc3F)cc12] |
| PFP-00144 | 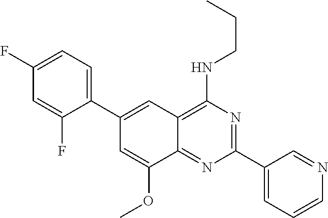 [SMILES: CCCNc1nc(-c2cccnc2)nc2c(OC)cc(-c3ccc(F)cc3F)cc12] |
| PFP-00145 | 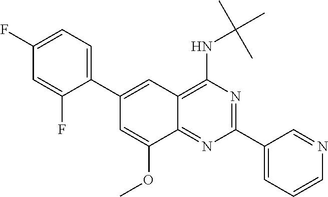 [SMILES: COc1cc(-c2ccc(F)cc2F)cc2c(NC(C)(C)C)nc(-c3cccnc3)nc12] |
| PFP-00146 | 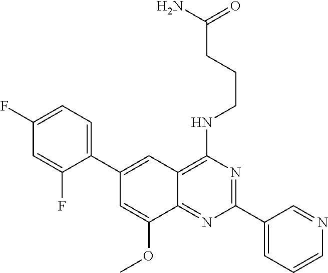 [SMILES: COc1cc(-c2ccc(F)cc2F)cc2c(NCCCC(N)=O)nc(-c3cccnc3)nc12] |
| PFP-00147 | 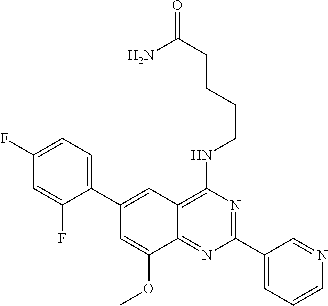 [SMILES: COc1cc(-c2ccc(F)cc2F)cc2c(NCCCCC(N)=O)nc(-c3cccnc3)nc12] |
| PFP-00148 | 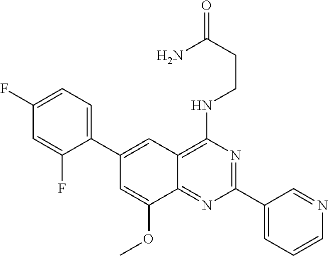 [SMILES: COc1cc(-c2ccc(F)cc2F)cc2c(NCCC(N)=O)nc(-c3cccnc3)nc12] |
| PFP-00149 | 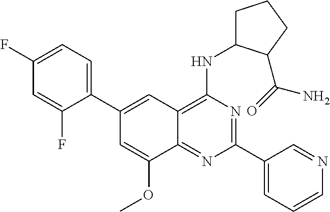 [SMILES: COc1cc(-c2ccc(F)cc2F)cc2c(NC3CCCC3C(N)=O)nc(-c3cccnc3)nc12] |
| PFP-00150 | 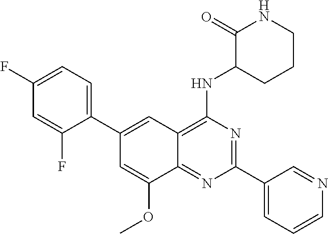 [SMILES: COc1cc(-c2ccc(F)cc2F)cc2c(NC3CCCNC3=O)nc(-c3cccnc3)nc12] |
| PFP-00151 | 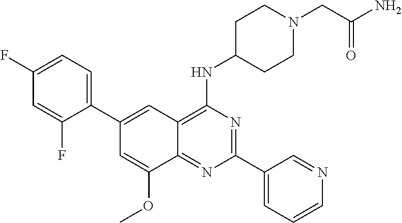 [SMILES: COc1cc(-c2ccc(F)cc2F)cc2c(NC3CCN(CC(N)=O)CC3)nc(-c3cccnc3)nc12] |
| PFP-00152 | 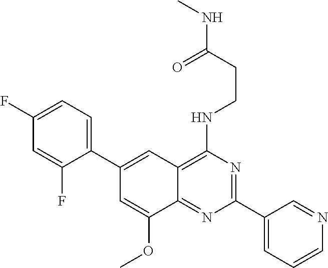 [SMILES: CNC(=O)CCNc1nc(-c2cccnc2)nc2c(OC)cc(-c3ccc(F)cc3F)cc12] |
| PFP-00153 | 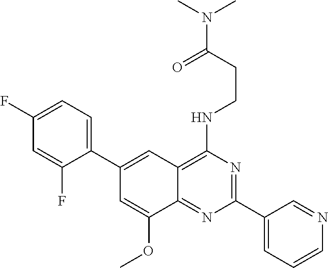 [SMILES: COc1cc(-c2ccc(F)cc2F)cc2c(NCCC(=O)N(C)C)nc(-c3cccnc3)nc12] |
| PFP-00154 | 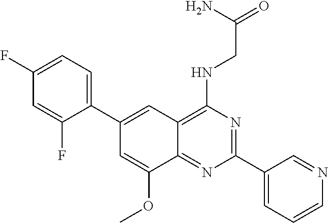 [SMILES: COc1cc(-c2ccc(F)cc2F)cc2c(NCC(N)=O)nc(-c3cccnc3)nc12] |
| PFP-00155 | 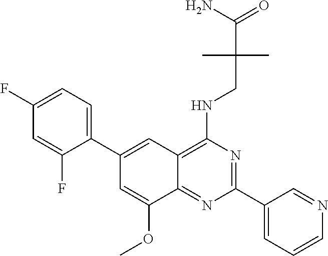 [SMILES: COc1cc(-c2ccc(F)cc2F)cc2c(NCC(C)(C)C(N)=O)nc(-c3cccnc3)nc12] |
| PFP-00156 | 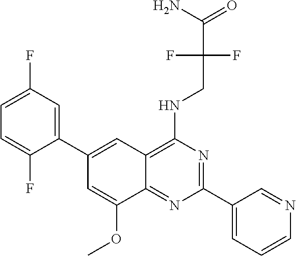 [SMILES: COc1cc(-c2cc(F)ccc2F)cc2c(NCC(F)(F)C(N)=O)nc(-c3cccnc3)nc12] |
| PFP-00157 | 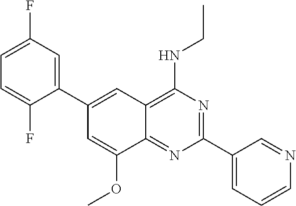 [SMILES: CCNc1nc(-c2cccnc2)nc2c(OC)cc(-c3cc(F)ccc3F)cc12] |
| PFP-00158 | 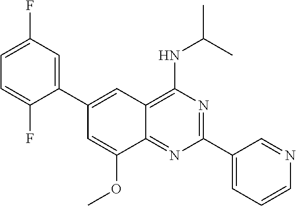 [SMILES: COc1cc(-c2cc(F)ccc2F)cc2c(NC(C)C)nc(-c3cccnc3)nc12] |
| PFP-00159 | 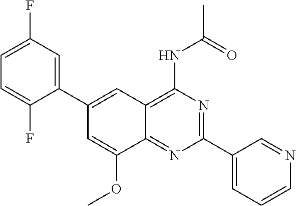 [SMILES: COc1cc(-c2cc(F)ccc2F)cc2c(NC(C)=O)nc(-c3cccnc3)nc12] |
| PFP-00160 | 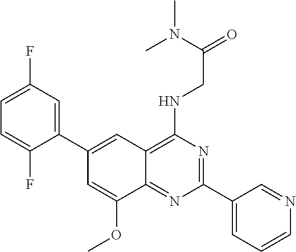 [SMILES: COc1cc(-c2cc(F)ccc2F)cc2c(NCC(=O)N(C)C)nc(-c3cccnc3)nc12] |
| PFP-00161 | 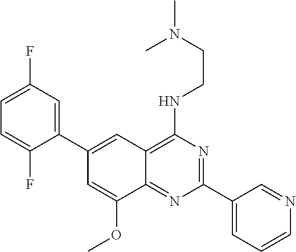 [SMILES: COc1cc(-c2cc(F)ccc2F)cc2c(NCCN(C)C)nc(-c3cccnc3)nc12] |
| PFP-00162 | 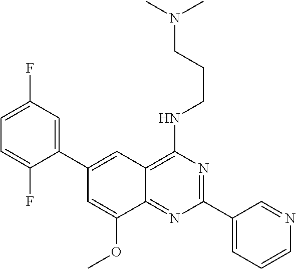 [SMILES: COc1cc(-c2cc(F)ccc2F)cc2c(NCCCN(C)C)nc(-c3cccnc3)nc12] |
| PFP-00163 | 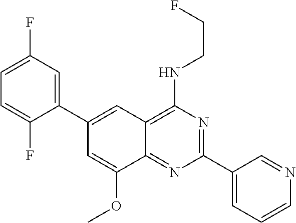 [SMILES: COc1cc(-c2cc(F)ccc2F)cc2c(NCCF)nc(-c3cccnc3)nc12] |
| PFP-00164 | 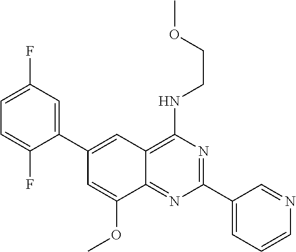 [SMILES: COCCNc1nc(-c2cccnc2)nc2c(OC)cc(-c3cc(F)ccc3F)cc12] |
| PFP-00165 | 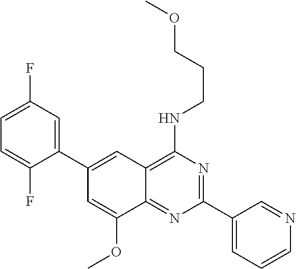 [SMILES: COCCCNc1nc(-c2cccnc2)nc2c(OC)cc(-c3cc(F)ccc3F)cc12] |
| PFP-00166 | 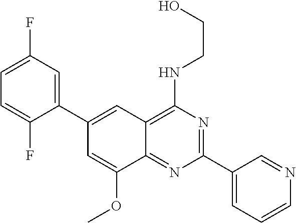 [SMILES: COc1cc(-c2cc(F)ccc2F)cc2c(NCCO)nc(-c3cccnc3)nc12] |
| PFP-00167 | 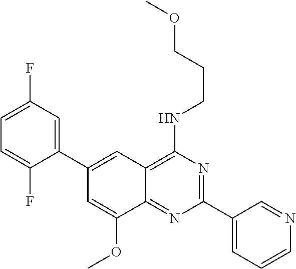 [SMILES: COCCCNc1nc(-c2cccnc2)nc2c(OC)cc(-c3cc(F)ccc3F)cc12] |
| PFP-00168 | 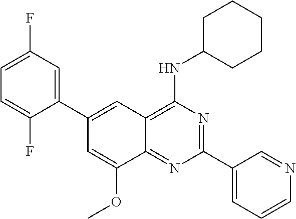 [SMILES: COc1cc(-c2cc(F)ccc2F)cc2c(NC3CCCCC3)nc(-c3cccnc3)nc12] |
| PFP-00169 | 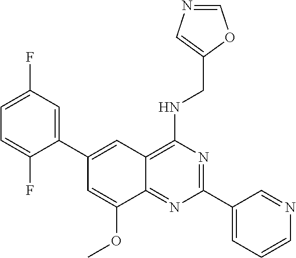 [SMILES: COc1cc(-c2cc(F)ccc2F)cc2c(NCc3cnco3)nc(-c3cccnc3)nc12] |
| PFP-00170 | 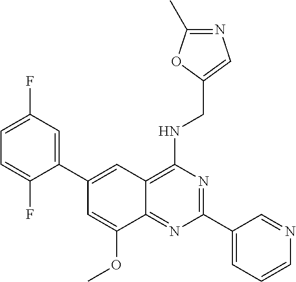 [SMILES: COc1cc(-c2cc(F)ccc2F)cc2c(NCc3cnc(C)o3)nc(-c3cccnc3)nc12] |
| PFP-00171 | 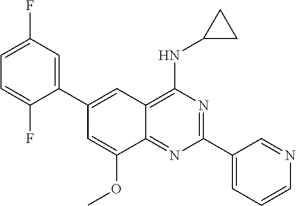 [SMILES: COc1cc(-c2cc(F)ccc2F)cc2c(NC3CC3)nc(-c3cccnc3)nc12] |
| PFP-00172 | 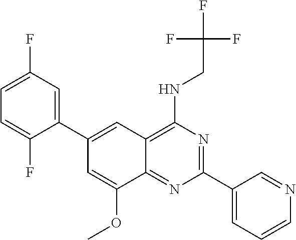 [SMILES: COc1cc(-c2cc(F)ccc2F)cc2c(NCC(F)(F)F)nc(-c3cccnc3)nc12] |
| PFP-00173 | 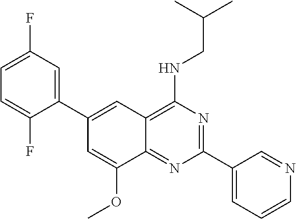 [SMILES: COc1cc(-c2cc(F)ccc2F)cc2c(NCC(C)C)nc(-c3cccnc3)nc12] |
| PFP-00174 | 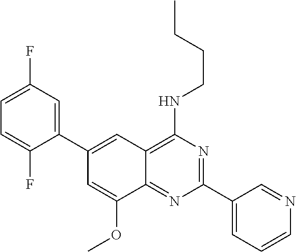 [SMILES: CCCCNc1nc(-c2cccnc2)nc2c(OC)cc(-c3cc(F)ccc3F)cc12] |
| PFP-00175 | 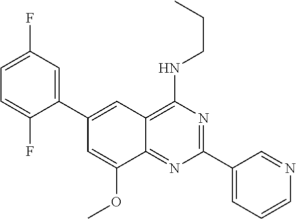 [SMILES: CCCNc1nc(-c2cccnc2)nc2c(OC)cc(-c3cc(F)ccc3F)cc12] |
| PFP-00176 | 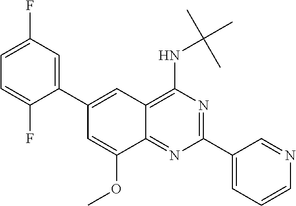 [SMILES: COc1cc(-c2cc(F)ccc2F)cc2c(NC(C)(C)C)nc(-c3cccnc3)nc12] |
| PFP-00177 | 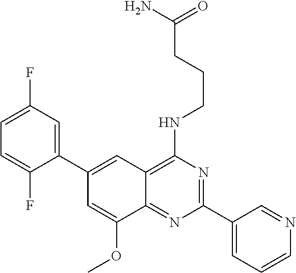 [SMILES: COc1cc(-c2cc(F)ccc2F)cc2c(NCCCC(N)=O)nc(-c3cccnc3)nc12] |
| PFP-00178 | 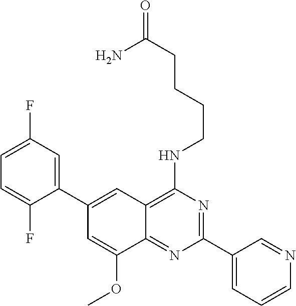 [SMILES: COc1cc(-c2cc(F)ccc2F)cc2c(NCCCCC(N)=O)nc(-c3cccnc3)nc12] |
| PFP-00179 | 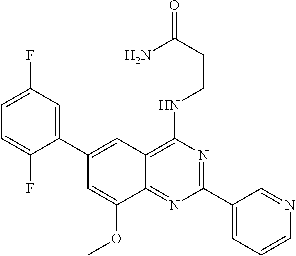 [SMILES: COc1cc(-c2cc(F)ccc2F)cc2c(NCCC(N)=O)nc(-c3cccnc3)nc12] |
| PFP-00180 | 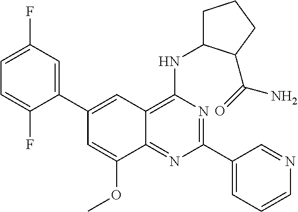 [SMILES: COc1cc(-c2cc(F)ccc2F)cc2c(NC3CCCC3C(N)=O)nc(-c3cccnc3)nc12] |
| PFP-00181 | 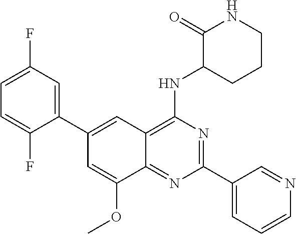 [SMILES: COc1cc(-c2cc(F)ccc2F)cc2c(NC3CCCNC3=O)nc(-c3cccnc3)nc12] |
| PFP-00182 | 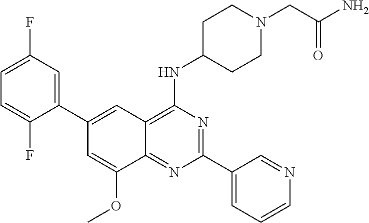 [SMILES: COc1cc(-c2cc(F)ccc2F)cc2c(NC3CCN(CC(N)=O)CC3)nc(-c3cccnc3)nc12] |
| PFP-00183 | 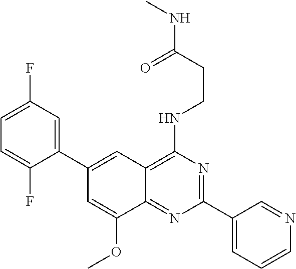 [SMILES: CNC(=O)CCNc1nc(-c2cccnc2)nc2c(OC)cc(-c3cc(F)ccc3F)cc12] |
| PFP-00184 | 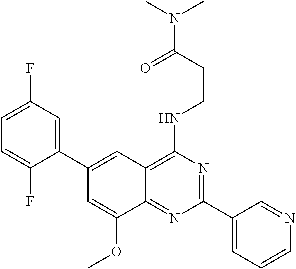 [SMILES: COc1cc(-c2cc(F)ccc2F)cc2c(NCCC(=O)N(C)C)nc(-c3cccnc3)nc12] |
| PFP-00185 | 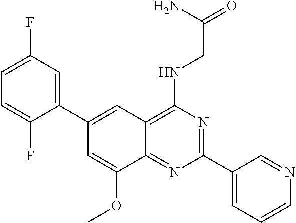 [SMILES: COc1cc(-c2cc(F)ccc2F)cc2c(NCC(N)=O)nc(-c3cccnc3)nc12] |
| PFP-00186 | 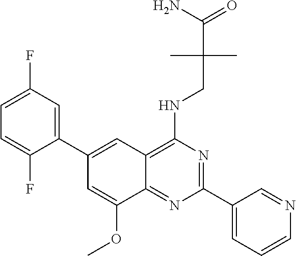 [SMILES: COc1cc(-c2cc(F)ccc2F)cc2c(NCC(C)(C)C(N)=O)nc(-c3cccnc3)nc12] |
| PFP-00187 | 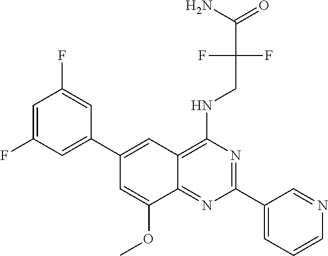 [SMILES: COc1cc(-c2cc(F)cc(F)c2)cc2c(NCC(F)(F)C(N)=O)nc(-c3cccnc3)nc12] |
| PFP-00188 | 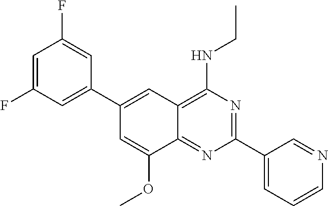 [SMILES: CCNc1nc(-c2cccnc2)nc2c(OC)cc(-c3cc(F)cc(F)c3)cc12] |
| PFP-00189 | 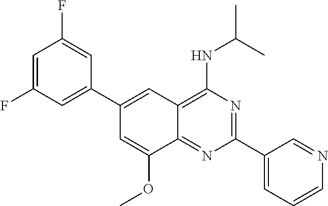 [SMILES: COc1cc(-c2cc(F)cc(F)c2)cc2c(NC(C)C)nc(-c3cccnc3)nc12] |
| PFP-00190 | 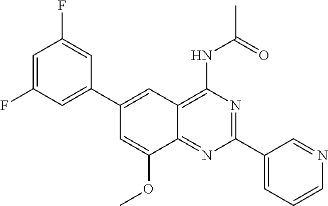 [SMILES: COc1cc(-c2cc(F)cc(F)c2)cc2c(NC(C)=O)nc(-c3cccnc3)nc12] |
| PFP-00191 | 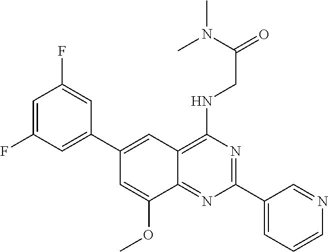 [SMILES: COc1cc(-c2cc(F)cc(F)c2)cc2c(NCC(=O)N(C)C)nc(-c3cccnc3)nc12] |
| PFP-00192 | 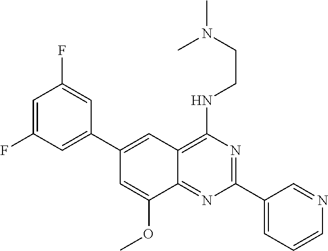 [SMILES: COc1cc(-c2cc(F)cc(F)c2)cc2c(NCCN(C)C)nc(-c3cccnc3)nc12] |
| PFP-00193 | 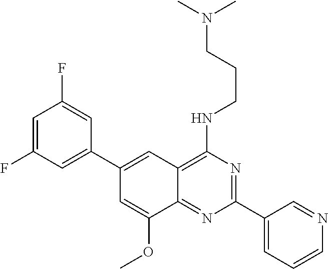 [SMILES: COc1cc(-c2cc(F)cc(F)c2)cc2c(NCCCN(C)C)nc(-c3cccnc3)nc12] |
| PFP-00194 | 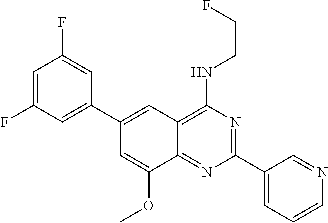 [SMILES: COc1cc(-c2cc(F)cc(F)c2)cc2c(NCCF)nc(-c3cccnc3)nc12] |
| PFP-00195 | 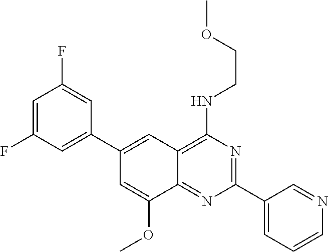 [SMILES: COCCNc1nc(-c2cccnc2)nc2c(OC)cc(-c3cc(F)cc(F)c3)cc12] |
| PFP-00196 | 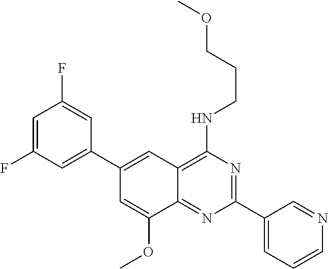 [SMILES: COCCCNc1nc(-c2cccnc2)nc2c(OC)cc(-c3cc(F)cc(F)c3)cc12] |
| PFP-00197 | 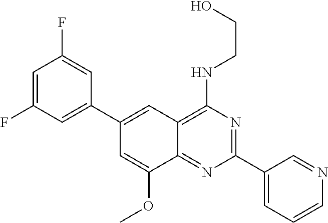 [SMILES: COc1cc(-c2cc(F)cc(F)c2)cc2c(NCCO)nc(-c3cccnc3)nc12] |
| PFP-00198 | 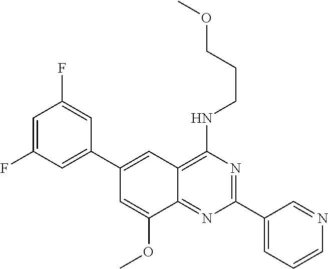 [SMILES: COCCCNc1nc(-c2cccnc2)nc2c(OC)cc(-c3cc(F)cc(F)c3)cc12] |
| PFP-00199 | 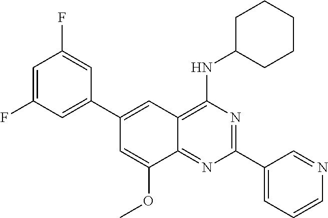 [SMILES: COc1cc(-c2cc(F)cc(F)c2)cc2c(NC3CCCCC3)nc(-c3cccnc3)nc12] |
| PFP-00200 | 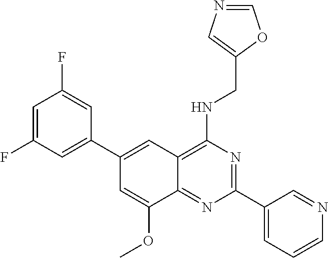 [SMILES: COc1cc(-c2cc(F)cc(F)c2)cc2c(NCc3cnco3)nc(-c3cccnc3)nc12] |
| PFP-00201 | 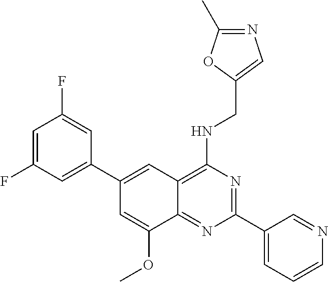 [SMILES: COc1cc(-c2cc(F)cc(F)c2)cc2c(NCc3cnc(C)o3)nc(-c3cccnc3)nc12] |
| PFP-00202 | 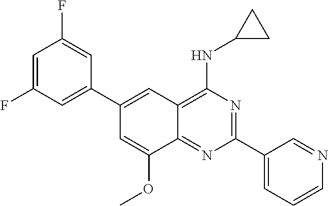 [SMILES: COc1cc(-c2cc(F)cc(F)c2)cc2c(NC3CC3)nc(-c3cccnc3)nc12] |
| PFP-00203 | 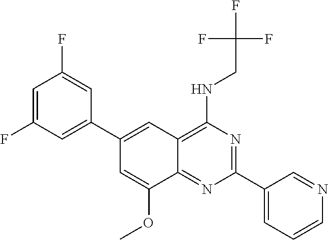 [SMILES: COc1cc(-c2cc(F)cc(F)c2)cc2c(NCC(F)(F)F)nc(-c3cccnc3)nc12] |
| PFP-00204 | 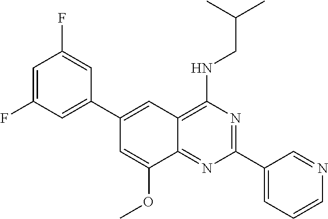 [SMILES: COc1cc(-c2cc(F)cc(F)c2)cc2c(NCC(C)C)nc(-c3cccnc3)nc12] |
| PFP-00205 | 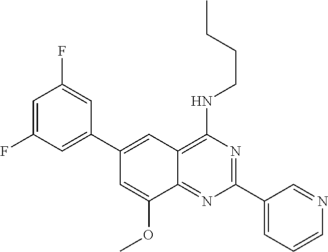 [SMILES: CCCCNc1nc(-c2cccnc2)nc2c(OC)cc(-c3cc(F)cc(F)c3)cc12] |
| PFP-00206 | 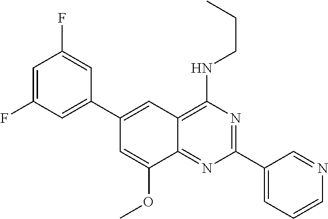 [SMILES: CCCNc1nc(-c2cccnc2)nc2c(OC)cc(-c3cc(F)cc(F)c3)cc12] |
| PFP-00207 | 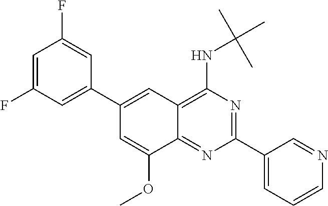 [SMILES: COc1cc(-c2cc(F)cc(F)c2)cc2c(NC(C)(C)C)nc(-c3cccnc3)nc12] |
| PFP-00208 | 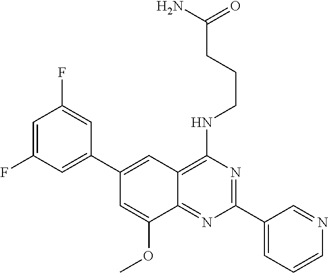 [SMILES: COc1cc(-c2cc(F)cc(F)c2)cc2c(NCCCC(N)=O)nc(-c3cccnc3)nc12] |
| PFP-00209 | 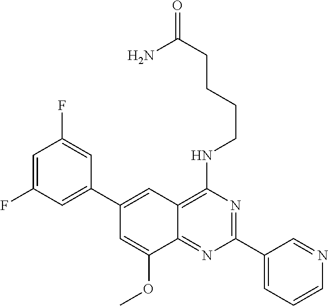 [SMILES: COc1cc(-c2cc(F)cc(F)c2)cc2c(NCCCCC(N)=O)nc(-c3cccnc3)nc12] |
| PFP-00210 | 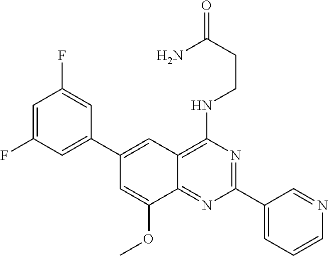 [SMILES: COc1cc(-c2cc(F)cc(F)c2)cc2c(NCCC(N)=O)nc(-c3cccnc3)nc12] |
| PFP-00211 | 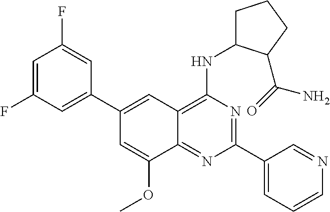 [SMILES: COc1cc(-c2cc(F)cc(F)c2)cc2c(NC3CCCC3C(N)=O)nc(-c3cccnc3)nc12] |
| PFP-00212 | 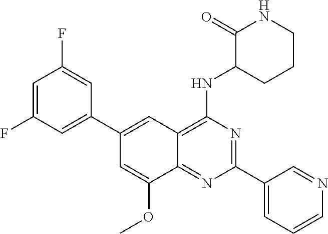 [SMILES: COc1cc(-c2cc(F)cc(F)c2)cc2c(NC3CCCNC3=O)nc(-c3cccnc3)nc12] |
| PFP-00213 | 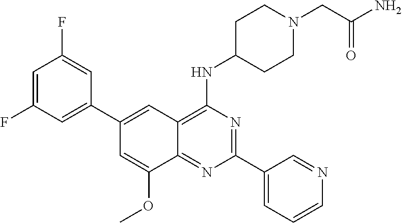 [SMILES: COc1cc(-c2cc(F)cc(F)c2)cc2c(NC3CCN(CC(N)=O)CC3)nc(-c3cccnc3)nc12] |
| PFP-00214 | 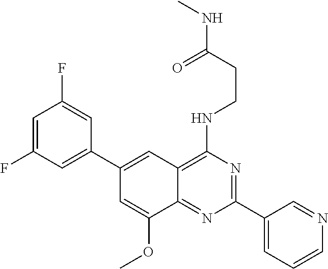 [SMILES: CNC(=O)CCNc1nc(-c2cccnc2)nc2c(OC)cc(-c3cc(F)cc(F)c3)cc12] |
| PFP-00215 | 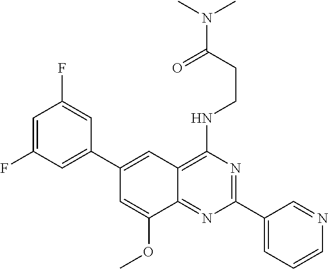 [SMILES: COc1cc(-c2cc(F)cc(F)c2)cc2c(NCCC(=O)N(C)C)nc(-c3cccnc3)nc12] |
| PFP-00216 | 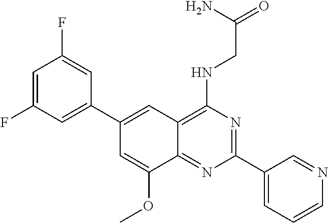 [SMILES: COc1cc(-c2cc(F)cc(F)c2)cc2c(NCC(N)=O)nc(-c3cccnc3)nc12] |
| PFP-00217 | 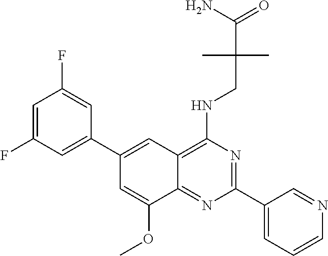 [SMILES: COc1cc(-c2cc(F)cc(F)c2)cc2c(NCC(C)(C)C(N)=O)nc(-c3cccnc3)nc12] |
| PFP-00218 | 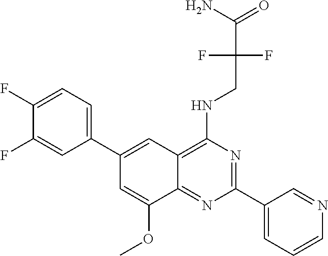 [SMILES: COc1cc(-c2ccc(F)c(F)c2)cc2c(NCC(F)(F)C(N)=O)nc(-c3cccnc3)nc12] |
| PFP-00219 | 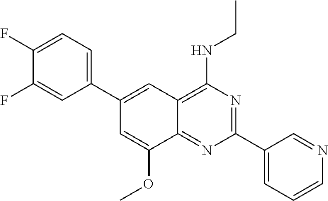 [SMILES: CCNc1nc(-c2cccnc2)nc2c(OC)cc(-c3ccc(F)c(F)c3)cc12] |
| PFP-00220 | 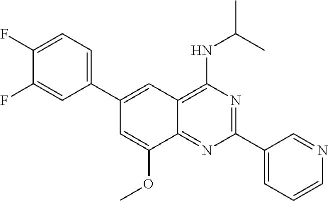 [SMILES: COc1cc(-c2ccc(F)c(F)c2)cc2c(NC(C)C)nc(-c3cccnc3)nc12] |
| PFP-00221 | 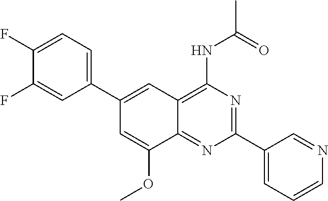 [SMILES: COc1cc(-c2ccc(F)c(F)c2)cc2c(NC(C)=O)nc(-c3cccnc3)nc12] |
| PFP-00222 | 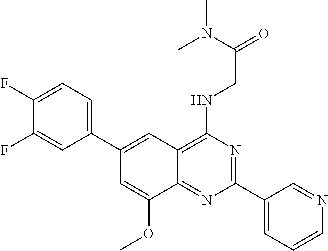 [SMILES: COc1cc(-c2ccc(F)c(F)c2)cc2c(NCC(=O)N(C)C)nc(-c3cccnc3)nc12] |
| PFP-00223 | 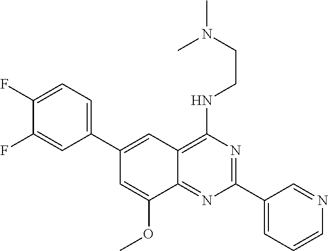 [SMILES: COc1cc(-c2ccc(F)c(F)c2)cc2c(NCCN(C)C)nc(-c3cccnc3)nc12] |
| PFP-00224 | 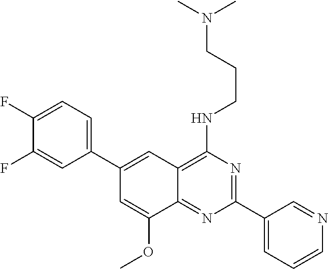 [SMILES: COc1cc(-c2ccc(F)c(F)c2)cc2c(NCCCN(C)C)nc(-c3cccnc3)nc12] |
| PFP-00225 | 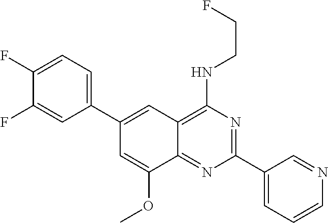 [SMILES: COc1cc(-c2ccc(F)c(F)c2)cc2c(NCCF)nc(-c3cccnc3)nc12] |
| PFP-00226 | 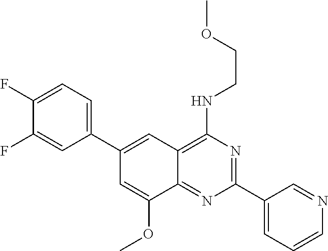 [SMILES: COCCNc1nc(-c2cccnc2)nc2c(OC)cc(-c3ccc(F)c(F)c3)cc12] |
| PFP-00227 | 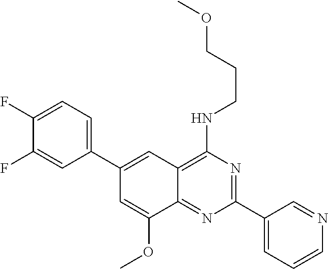 [SMILES: COCCCNc1nc(-c2cccnc2)nc2c(OC)cc(-c3ccc(F)c(F)c3)cc12] |
| PFP-00228 | 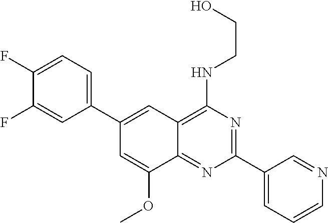 [SMILES: COc1cc(-c2ccc(F)c(F)c2)cc2c(NCCO)nc(-c3cccnc3)nc12] |
| PFP-00229 | 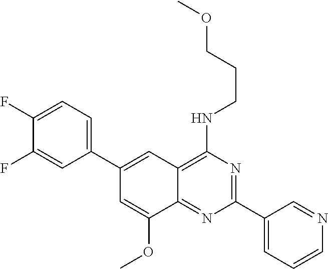 [SMILES: COCCCNc1nc(-c2cccnc2)nc2c(OC)cc(-c3ccc(F)c(F)c3)cc12] |
| PFP-00230 | 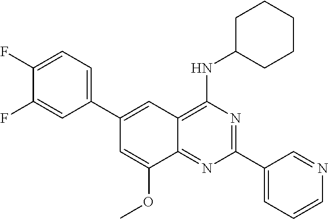 [SMILES: COc1cc(-c2ccc(F)c(F)c2)cc2c(NC3CCCCC3)nc(-c3cccnc3)nc12] |
| PFP-00231 | 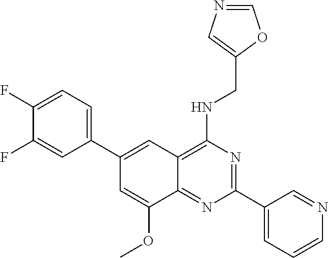 [SMILES: COc1cc(-c2ccc(F)c(F)c2)cc2c(NCc3cnco3)nc(-c3cccnc3)nc12] |
| PFP-00232 | 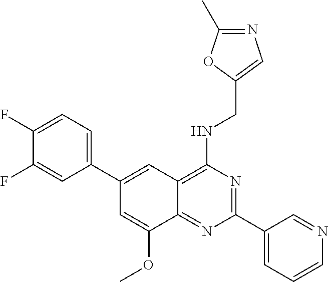 [SMILES: COc1cc(-c2ccc(F)c(F)c2)cc2c(NCc3cnc(C)o3)nc(-c3cccnc3)nc12] |
| PFP-00233 | 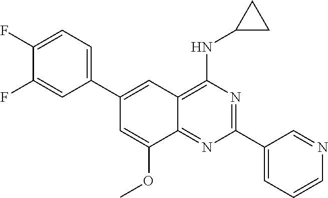 [SMILES: COc1cc(-c2ccc(F)c(F)c2)cc2c(NC3CC3)nc(-c3cccnc3)nc12] |
| PFP-00234 | 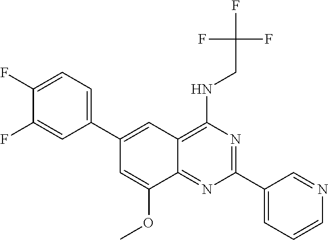 [SMILES: COc1cc(-c2ccc(F)c(F)c2)cc2c(NCC(F)(F)F)nc(-c3cccnc3)nc12] |
| PFP-00235 | 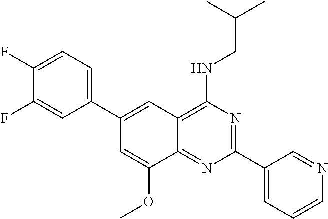 [SMILES: COc1cc(-c2ccc(F)c(F)c2)cc2c(NCC(C)C)nc(-c3cccnc3)nc12] |
| PFP-00236 | 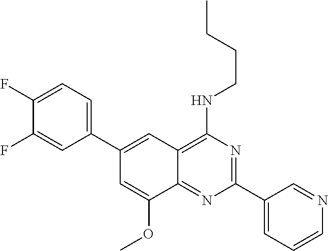 [SMILES: CCCCNc1nc(-c2cccnc2)nc2c(OC)cc(-c3ccc(F)c(F)c3)cc12] |
| PFP-00237 | 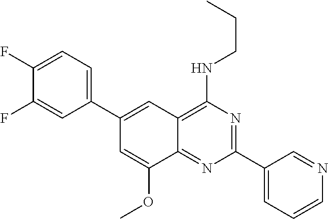 [SMILES: CCCNc1nc(-c2cccnc2)nc2c(OC)cc(-c3ccc(F)c(F)c3)cc12] |
| PFP-00238 | 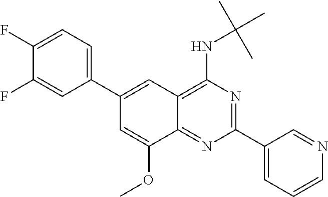 [SMILES: COc1cc(-c2ccc(F)c(F)c2)cc2c(NC(C)(C)C)nc(-c3cccnc3)nc12] |
| PFP-00239 | 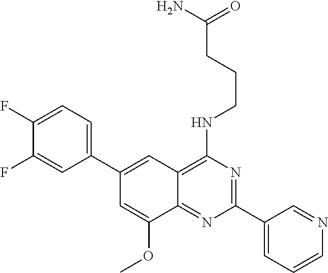 [SMILES: COc1cc(-c2ccc(F)c(F)c2)cc2c(NCCCC(N)=O)nc(-c3cccnc3)nc12] |
| PFP-00240 | 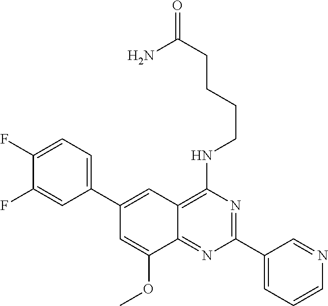 [SMILES: COc1cc(-c2ccc(F)c(F)c2)cc2c(NCCCCC(N)=O)nc(-c3cccnc3)nc12] |
| PFP-00241 | 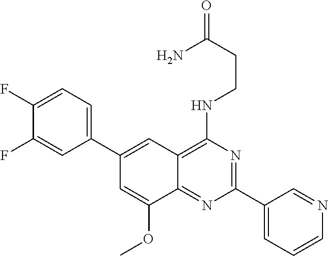 [SMILES: COc1cc(-c2ccc(F)c(F)c2)cc2c(NCCC(N)=O)nc(-c3cccnc3)nc12] |
| PFP-00242 | 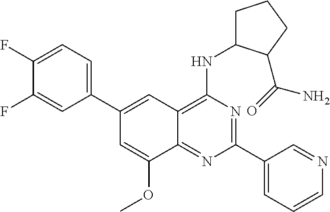 [SMILES: COc1cc(-c2ccc(F)c(F)c2)cc2c(NC3CCCC3C(N)=O)nc(-c3cccnc3)nc12] |
| PFP-00243 | 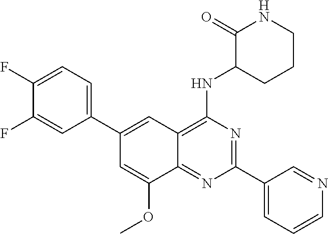 [SMILES: COc1cc(-c2ccc(F)c(F)c2)cc2c(NC3CCCNC3=O)nc(-c3cccnc3)nc12] |
| PFP-00244 | 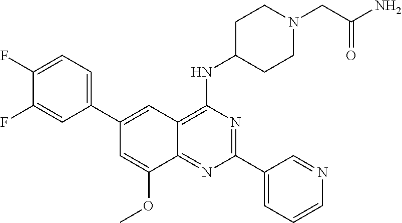 [SMILES: COc1cc(-c2ccc(F)c(F)c2)cc2c(NC3CCN(CC(N)=O)CC3)nc(-c3cccnc3)nc12] |
| PFP-00245 | 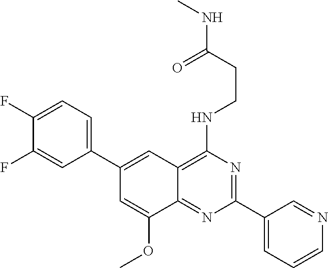 [SMILES: CNC(=O)CCNc1nc(-c2cccnc2)nc2c(OC)cc(-c3ccc(F)c(F)c3)cc12] |
| PFP-00246 | 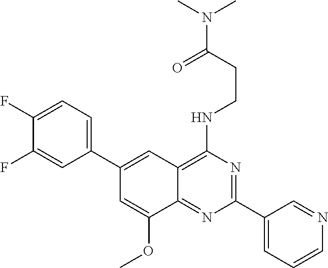 [SMILES: COc1cc(-c2ccc(F)c(F)c2)cc2c(NCCC(=O)N(C)C)nc(-c3cccnc3)nc12] |
| PFP-00247 | 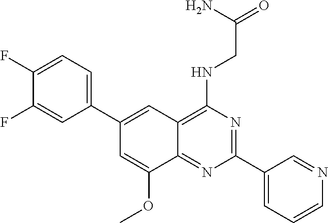 [SMILES: COc1cc(-c2ccc(F)c(F)c2)cc2c(NCC(N)=O)nc(-c3cccnc3)nc12] |
| PFP-00248 | 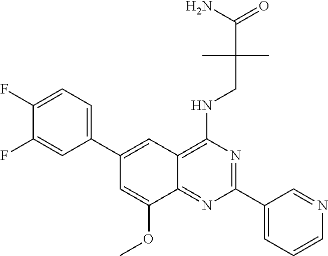 [SMILES: COc1cc(-c2ccc(F)c(F)c2)cc2c(NCC(C)(C)C(N)=O)nc(-c3cccnc3)nc12] |
| PFP-00249 | 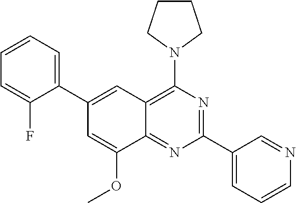 [SMILES: COc1cc(-c2ccccc2F)cc2c(N3CCCC3)nc(-c3cccnc3)nc12] |
| PFP-00250 | 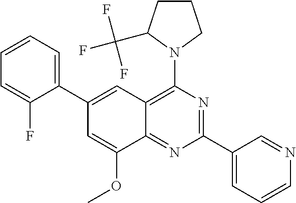 [SMILES: COc1cc(-c2ccccc2F)cc2c(N3CCCC3C(F)(F)F)nc(-c3cccnc3)nc12] |
| PFP-00251 | 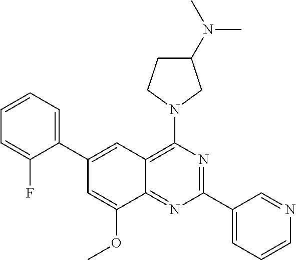 [SMILES: COc1cc(-c2ccccc2F)cc2c(N3CCC(N(C)C)C3)nc(-c3cccnc3)nc12] |
| PFP-00252 | 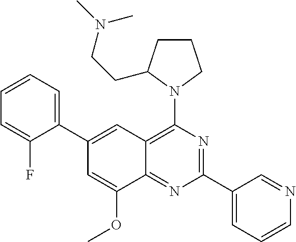 [SMILES: COc1cc(-c2ccccc2F)cc2c(N3CCCC3CCN(C)C)nc(-c3cccnc3)nc12] |
| PFP-00253 | 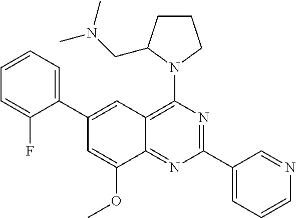 [SMILES: COc1cc(-c2ccccc2F)cc2c(N3CCCC3CN(C)C)nc(-c3cccnc3)nc12] |
| PFP-00254 | 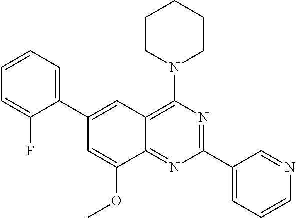 [SMILES: COc1cc(-c2ccccc2F)cc2c(N3CCCCC3)nc(-c3cccnc3)nc12] |
| PFP-00255 | 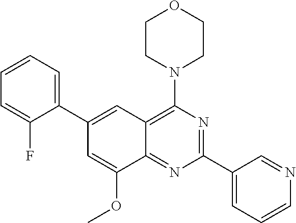 [SMILES: COc1cc(-c2ccccc2F)cc2c(N3CCOCC3)nc(-c3cccnc3)nc12] |
| PFP-00256 | 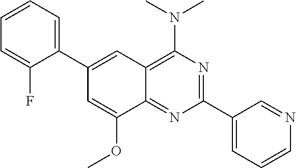 [SMILES: COc1cc(-c2ccccc2F)cc2c(N(C)C)nc(-c3cccnc3)nc12] |
| PFP-00257 | 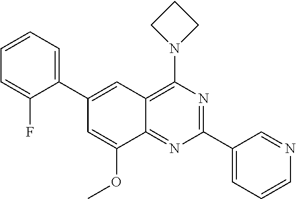 [SMILES: COc1cc(-c2ccccc2F)cc2c(N3CCC3)nc(-c3cccnc3)nc12] |
| PFP-00258 | 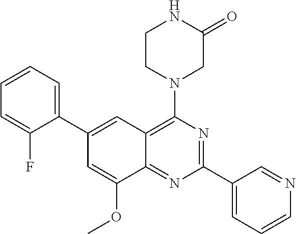 [SMILES: COc1cc(-c2ccccc2F)cc2c(N3CCNC(=O)C3)nc(-c3cccnc3)nc12] |
| PFP-00259 | 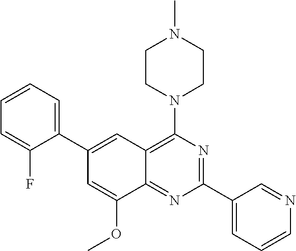 [SMILES: COc1cc(-c2ccccc2F)cc2c(N3CCN(C)CC3)nc(-c3cccnc3)nc12] |
| PFP-00260 | 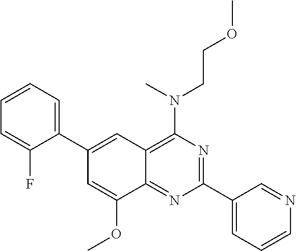 [SMILES: COCCN(C)c1nc(-c2cccnc2)nc2c(OC)cc(-c3ccccc3F)cc12] |
| PFP-00261 | 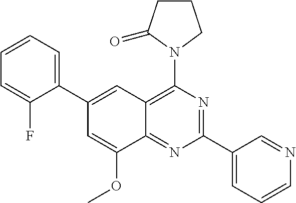 [SMILES: COc1cc(-c2ccccc2F)cc2c(N3CCCC3=O)nc(-c3cccnc3)nc12] |
| PFP-00262 | 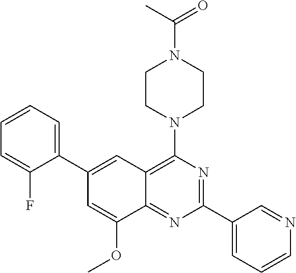 [SMILES: COc1cc(-c2ccccc2F)cc2c(N3CCN(C(C)=O)CC3)nc(-c3cccnc3)nc12] |
| PFP-00263 | 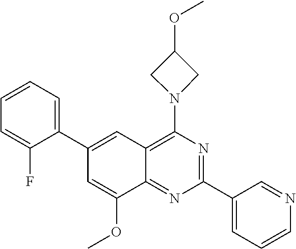 [SMILES: COc1cc(-c2ccccc2F)cc2c(N3CC(OC)C3)nc(-c3cccnc3)nc12] |
| PFP-00264 | 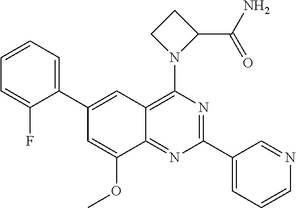 [SMILES: COc1cc(-c2ccccc2F)cc2c(N3CCC3C(N)=O)nc(-c3cccnc3)nc12] |
| PFP-00265 | 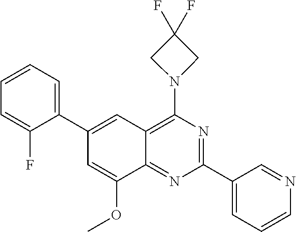 [SMILES: COc1cc(-c2ccccc2F)cc2c(N3CC(F)(F)C3)nc(-c3cccnc3)nc12] |
| PFP-00266 | 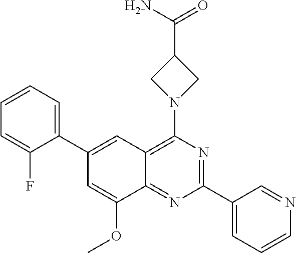 [SMILES: COc1cc(-c2ccccc2F)cc2c(N3CC(C(N)=O)C3)nc(-c3cccnc3)nc12] |
| PFP-00267 | 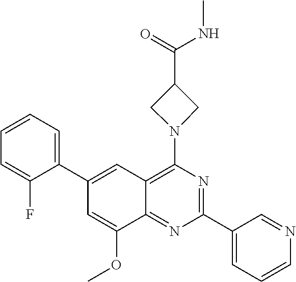 [SMILES: CNC(=O)C1CN(c2nc(-c3cccnc3)nc3c(OC)cc(-c4ccccc4F)cc23)C1] |
| PFP-00268 | 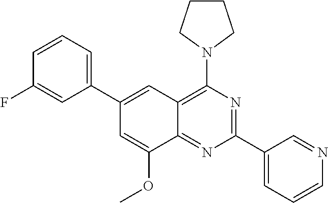 [SMILES: COc1cc(-c2cccc(F)c2)cc2c(N3CCCC3)nc(-c3cccnc3)nc12] |
| PFP-00269 | 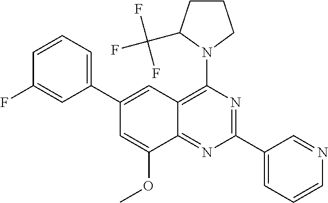 [SMILES: COc1cc(-c2cccc(F)c2)cc2c(N3CCCC3C(F)(F)F)nc(-c3cccnc3)nc12] |
| PFP-00270 | 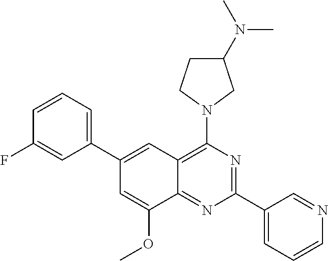 [SMILES: COc1cc(-c2cccc(F)c2)cc2c(N3CCC(N(C)C)C3)nc(-c3cccnc3)nc12] |
| PFP-00271 | 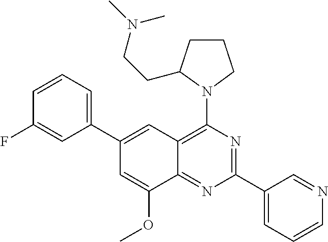 [SMILES: COc1cc(-c2cccc(F)c2)cc2c(N3CCCC3CCN(C)C)nc(-c3cccnc3)nc12] |
| PFP-00272 | 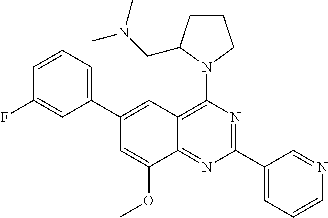 [SMILES: COc1cc(-c2cccc(F)c2)cc2c(N3CCCC3CN(C)C)nc(-c3cccnc3)nc12] |
| PFP-00273 | 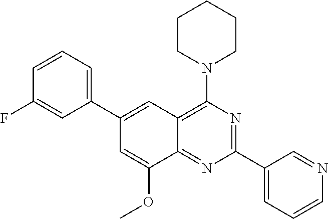 [SMILES: COc1cc(-c2cccc(F)c2)cc2c(N3CCCCC3)nc(-c3cccnc3)nc12] |
| PFP-00274 | 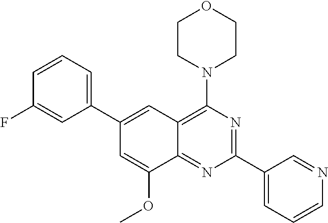 [SMILES: COc1cc(-c2cccc(F)c2)cc2c(N3CCOCC3)nc(-c3cccnc3)nc12] |
| PFP-00275 | 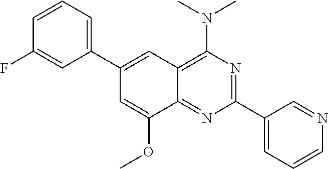 [SMILES: COc1cc(-c2cccc(F)c2)cc2c(N(C)C)nc(-c3cccnc3)nc12] |
| PFP-00276 | 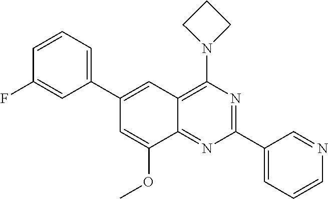 [SMILES: COc1cc(-c2cccc(F)c2)cc2c(N3CCC3)nc(-c3cccnc3)nc12] |
| PFP-00277 | 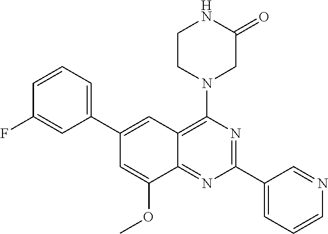 [SMILES: COc1cc(-c2cccc(F)c2)cc2c(N3CCNC(=O)C3)nc(-c3cccnc3)nc12] |
| PFP-00278 | 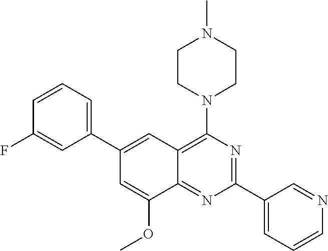 [SMILES: COc1cc(-c2cccc(F)c2)cc2c(N3CCN(C)CC3)nc(-c3cccnc3)nc12] |
| PFP-00279 | 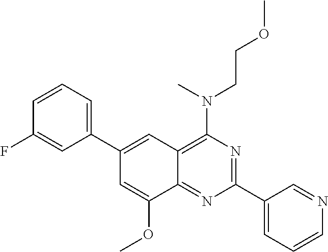 [SMILES: COCCN(C)c1nc(-c2cccnc2)nc2c(OC)cc(-c3cccc(F)c3)cc12] |
| PFP-00280 | 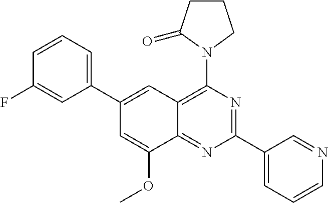 [SMILES: COc1cc(-c2cccc(F)c2)cc2c(N3CCCC3=O)nc(-c3cccnc3)nc12] |
| PFP-00281 | 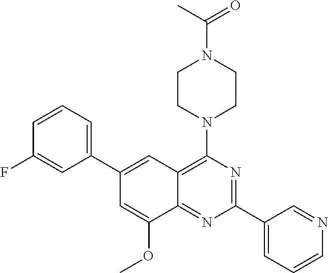 [SMILES: COc1cc(-c2cccc(F)c2)cc2c(N3CCN(C(C)=O)CC3)nc(-c3cccnc3)nc12] |
| PFP-00282 | 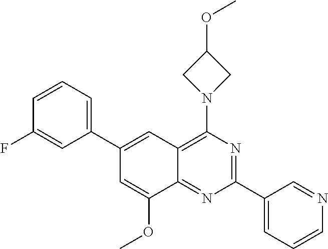 [SMILES: COc1cc(-c2cccc(F)c2)cc2c(N3CC(OC)C3)nc(-c3cccnc3)nc12] |
| PFP-00283 | 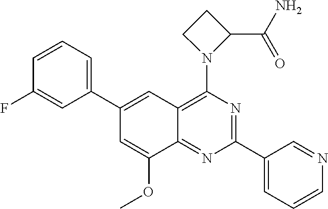 [SMILES: COc1cc(-c2cccc(F)c2)cc2c(N3CCC3C(N)=O)nc(-c3cccnc3)nc12] |
| PFP-00284 | 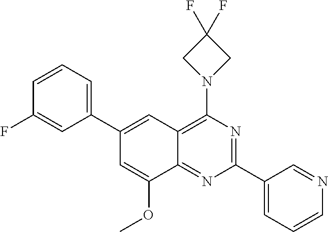 [SMILES: COc1cc(-c2cccc(F)c2)cc2c(N3CC(F)(F)C3)nc(-c3cccnc3)nc12] |
| PFP-00285 | 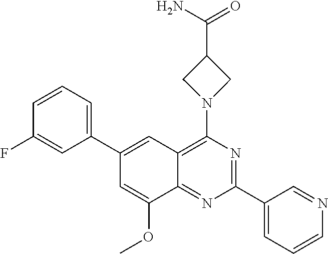 [SMILES: COc1cc(-c2cccc(F)c2)cc2c(N3CC(C(N)=O)C3)nc(-c3cccnc3)nc12] |
| PFP-00286 | 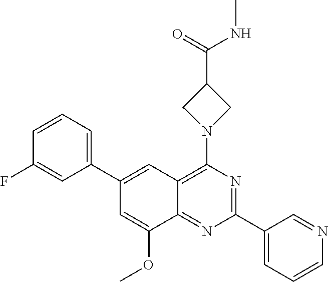 [SMILES: CNC(=O)C1CN(c2nc(-c3cccnc3)nc3c(OC)cc(-c4cccc(F)c4)cc23)C1] |
| PFP-00287 | 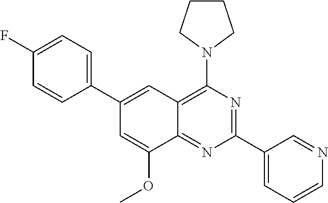 [SMILES: COc1cc(-c2ccc(F)cc2)cc2c(N3CCCC3)nc(-c3cccnc3)nc12] |
| PFP-00288 | 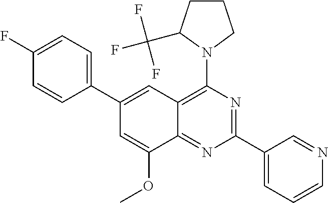 [SMILES: COc1cc(-c2ccc(F)cc2)cc2c(N3CCCC3C(F)(F)F)nc(-c3cccnc3)nc12] |
| PFP-00289 | 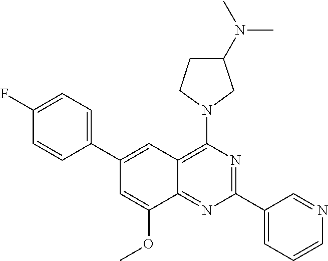 [SMILES: COc1cc(-c2ccc(F)cc2)cc2c(N3CCC(N(C)C)C3)nc(-c3cccnc3)nc12] |
| PFP-00290 | 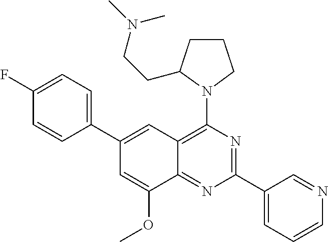 [SMILES: COc1cc(-c2ccc(F)cc2)cc2c(N3CCCC3CCN(C)C)nc(-c3cccnc3)nc12] |
| PFP-00291 | 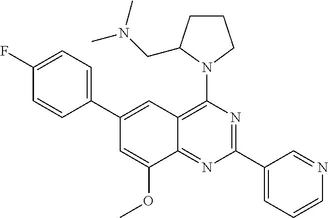 [SMILES: COc1cc(-c2ccc(F)cc2)cc2c(N3CCCC3CN(C)C)nc(-c3cccnc3)nc12] |
| PFP-00292 | 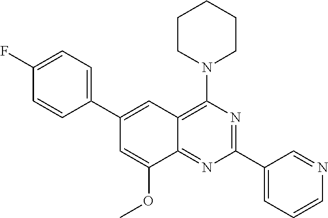 [SMILES: COc1cc(-c2ccc(F)cc2)cc2c(N3CCCCC3)nc(-c3cccnc3)nc12] |
| PFP-00293 | 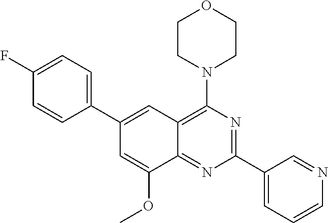 [SMILES: COc1cc(-c2ccc(F)cc2)cc2c(N3CCOCC3)nc(-c3cccnc3)nc12] |
| PFP-00294 | 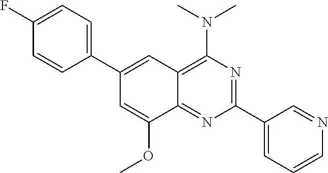 [SMILES: COc1cc(-c2ccc(F)cc2)cc2c(N(C)C)nc(-c3cccnc3)nc12] |
| PFP-00295 | 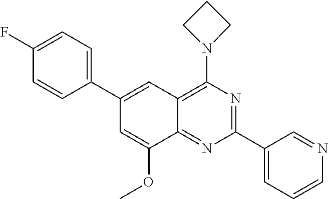 [SMILES: COc1cc(-c2ccc(F)cc2)cc2c(N3CCC3)nc(-c3cccnc3)nc12] |
| PFP-00296 | 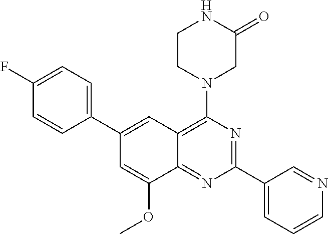 [SMILES: COc1cc(-c2ccc(F)cc2)cc2c(N3CCNC(=O)C3)nc(-c3cccnc3)nc12] |
| PFP-00297 | 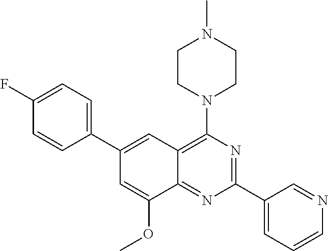 [SMILES: COc1cc(-c2ccc(F)cc2)cc2c(N3CCN(C)CC3)nc(-c3cccnc3)nc12] |
| PFP-00298 | 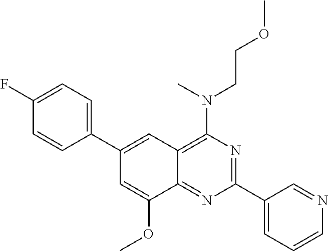 [SMILES: COCCN(C)c1nc(-c2cccnc2)nc2c(OC)cc(-c3ccc(F)cc3)cc12] |
| PFP-00299 | 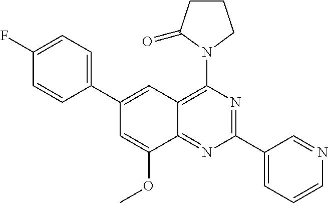 [SMILES: COc1cc(-c2ccc(F)cc2)cc2c(N3CCCC3=O)nc(-c3cccnc3)nc12] |
| PFP-00300 | 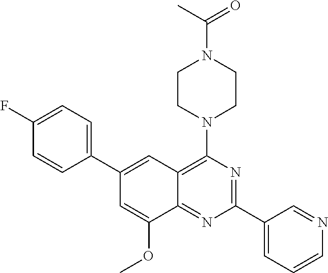 [SMILES: COc1cc(-c2ccc(F)cc2)cc2c(N3CCN(C(C)=O)CC3)nc(-c3cccnc3)nc12] |
| PFP-00301 | 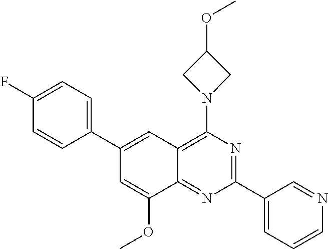 [SMILES: COc1cc(-c2ccc(F)cc2)cc2c(N3CC(OC)C3)nc(-c3cccnc3)nc12] |
| PFP-00302 | 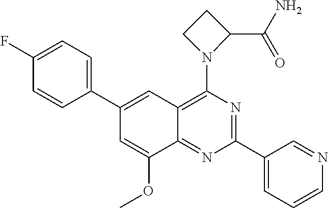 [SMILES: COc1cc(-c2ccc(F)cc2)cc2c(N3CCC3C(N)=O)nc(-c3cccnc3)nc12] |
| PFP-00303 | 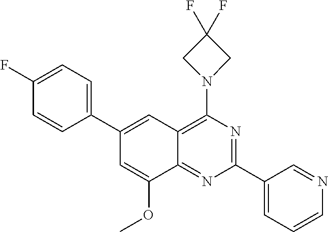 [SMILES: COc1cc(-c2ccc(F)cc2)cc2c(N3CC(F)(F)C3)nc(-c3cccnc3)nc12] |
| PFP-00304 | 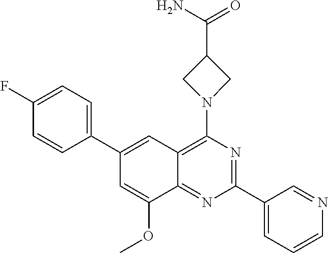 [SMILES: COc1cc(-c2ccc(F)cc2)cc2c(N3CC(C(N)=O)C3)nc(-c3cccnc3)nc12] |
| PFP-00305 | 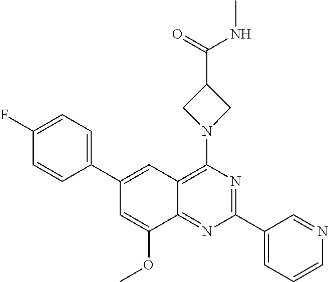 [SMILES: CNC(=O)C1CN(c2nc(-c3cccnc3)nc3c(OC)cc(-c4ccc(F)cc4)cc23)C1] |
| PFP-00306 | 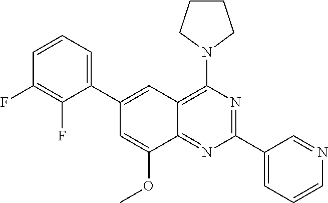 [SMILES: COc1cc(-c2cccc(F)c2F)cc2c(N3CCCC3)nc(-c3cccnc3)nc12] |
| PFP-00307 | 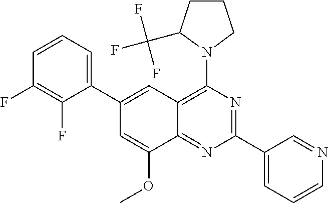 [SMILES: COc1cc(-c2cccc(F)c2F)cc2c(N3CCCC3C(F)(F)F)nc(-c3cccnc3)nc12] |
| PFP-00308 | 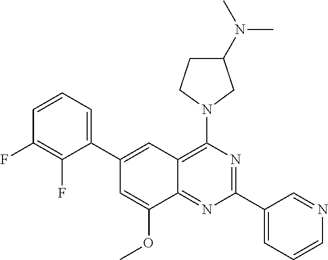 [SMILES: COc1cc(-c2cccc(F)c2F)cc2c(N3CCC(N(C)C)C3)nc(-c3cccnc3)nc12] |
| PFP-00309 | 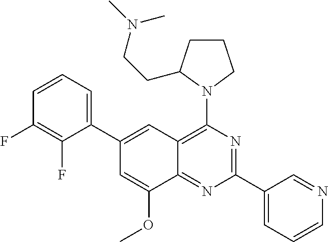 [SMILES: COc1cc(-c2cccc(F)c2F)cc2c(N3CCCC3CCN(C)C)nc(-c3cccnc3)nc12] |
| PFP-00310 | 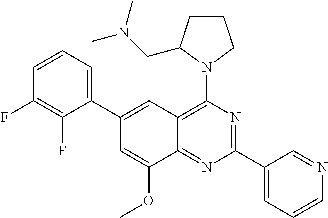 [SMILES: COc1cc(-c2cccc(F)c2F)cc2c(N3CCCC3CN(C)C)nc(-c3cccnc3)nc12] |
| PFP-00311 | 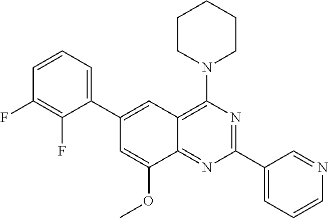 [SMILES: COc1cc(-c2cccc(F)c2F)cc2c(N3CCCCC3)nc(-c3cccnc3)nc12] |
| PFP-00312 | 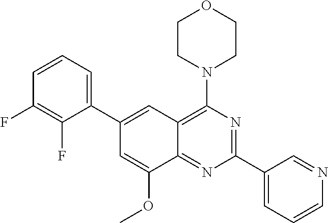 [SMILES: COc1cc(-c2cccc(F)c2F)cc2c(N3CCOCC3)nc(-c3cccnc3)nc12] |
| PFP-00313 | 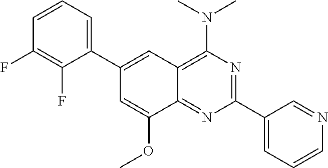 [SMILES: COc1cc(-c2cccc(F)c2F)cc2c(N(C)C)nc(-c3cccnc3)nc12] |
| PFP-00314 | 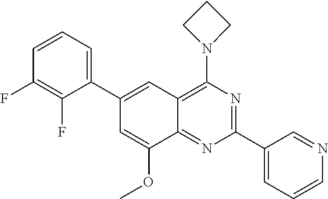 [SMILES: COc1cc(-c2cccc(F)c2F)cc2c(N3CCC3)nc(-c3cccnc3)nc12] |
| PFP-00315 | 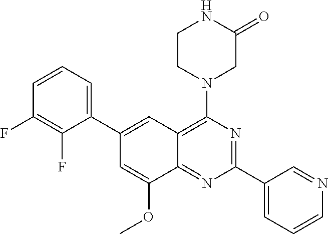 [SMILES: COc1cc(-c2cccc(F)c2F)cc2c(N3CCNC(=O)C3)nc(-c3cccnc3)nc12] |
| PFP-00316 | 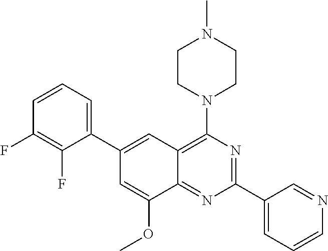 [SMILES: COc1cc(-c2cccc(F)c2F)cc2c(N3CCN(C)CC3)nc(-c3cccnc3)nc12] |
| PFP-00317 | 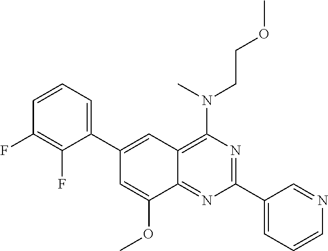 [SMILES: COCCN(C)c1nc(-c2cccnc2)nc2c(OC)cc(-c3cccc(F)c3F)cc12] |
| PFP-00318 | 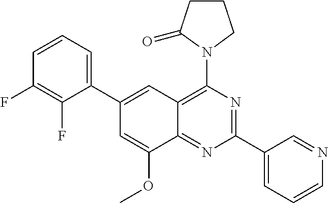 [SMILES: COc1cc(-c2cccc(F)c2F)cc2c(N3CCCC3=O)nc(-c3cccnc3)nc12] |
| PFP-00319 | 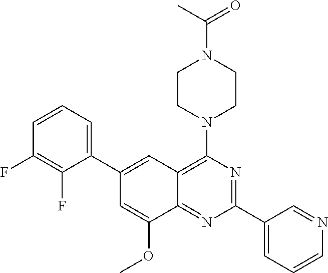 [SMILES: COc1cc(-c2cccc(F)c2F)cc2c(N3CCN(C(C)=O)CC3)nc(-c3cccnc3)nc12] |
| PFP-00320 | 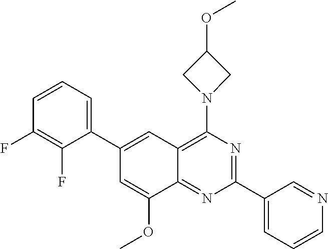 [SMILES: COc1cc(-c2cccc(F)c2F)cc2c(N3CC(OC)C3)nc(-c3cccnc3)nc12] |
| PFP-00321 | 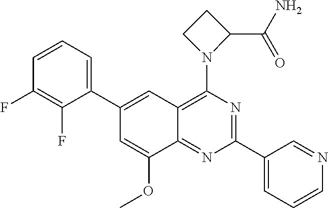 [SMILES: COc1cc(-c2cccc(F)c2F)cc2c(N3CCC3C(N)=O)nc(-c3cccnc3)nc12] |
| PFP-00322 | 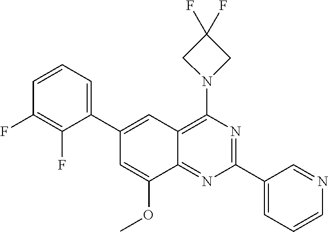 [SMILES: COc1cc(-c2cccc(F)c2F)cc2c(N3CC(F)(F)C3)nc(-c3cccnc3)nc12] |
| PFP-00323 | 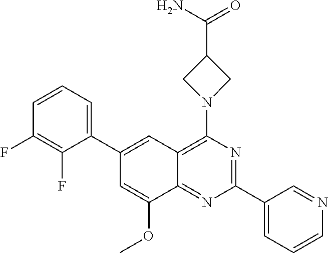 [SMILES: COc1cc(-c2cccc(F)c2F)cc2c(N3CC(C(N)=O)C3)nc(-c3cccnc3)nc12] |
| PFP-00324 | 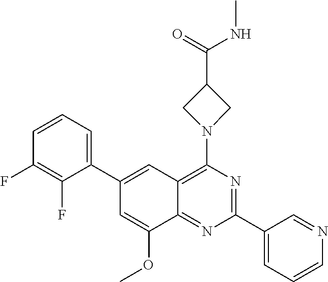 [SMILES: CNC(=O)C1CN(c2nc(-c3cccnc3)nc3c(OC)cc(-c4cccc(F)c4F)cc23)C1] |
| PFP-00325 | 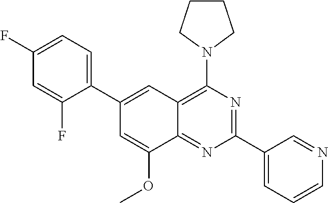 [SMILES: COc1cc(-c2ccc(F)cc2F)cc2c(N3CCCC3)nc(-c3cccnc3)nc12] |
| PFP-00326 | 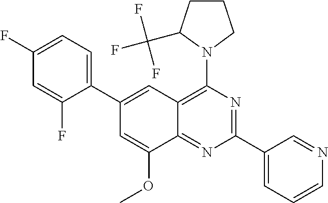 [SMILES: COc1cc(-c2ccc(F)cc2F)cc2c(N3CCCC3C(F)(F)F)nc(-c3cccnc3)nc12] |
| PFP-00327 | 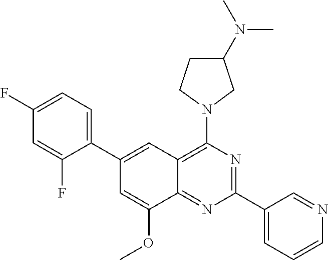 [SMILES: COc1cc(-c2ccc(F)cc2F)cc2c(N3CCC(N(C)C)C3)nc(-c3cccnc3)nc12] |
| PFP-00328 | 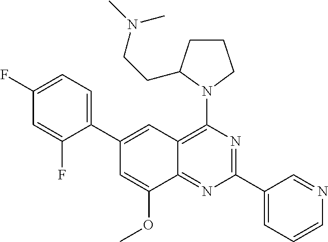 [SMILES: COc1cc(-c2ccc(F)cc2F)cc2c(N3CCCC3CCN(C)C)nc(-c3cccnc3)nc12] |
| PFP-00329 | 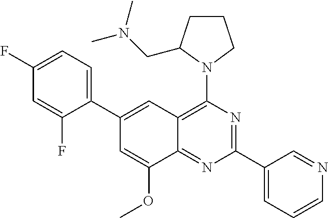 [SMILES: COc1cc(-c2ccc(F)cc2F)cc2c(N3CCCC3CN(C)C)nc(-c3cccnc3)nc12] |
| PFP-00330 | 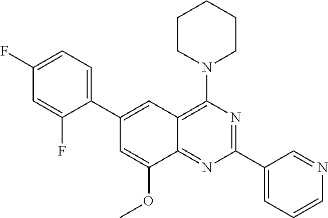 [SMILES: COc1cc(-c2ccc(F)cc2F)cc2c(N3CCCCC3)nc(-c3cccnc3)nc12] |
| PFP-00331 | 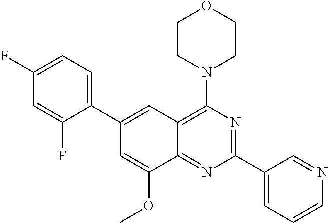 [SMILES: COc1cc(-c2ccc(F)cc2F)cc2c(N3CCOCC3)nc(-c3cccnc3)nc12] |
| PFP-00332 | 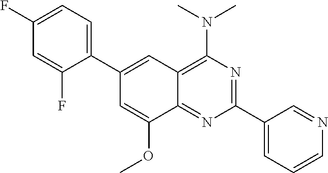 [SMILES: COc1cc(-c2ccc(F)cc2F)cc2c(N(C)C)nc(-c3cccnc3)nc12] |
| PFP-00333 | 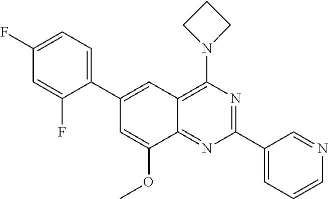 [SMILES: COc1cc(-c2ccc(F)cc2F)cc2c(N3CCC3)nc(-c3cccnc3)nc12] |
| PFP-00334 | 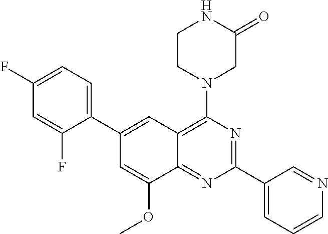 [SMILES: COc1cc(-c2ccc(F)cc2F)cc2c(N3CCNC(=O)C3)nc(-c3cccnc3)nc12] |
| PFP-00335 | 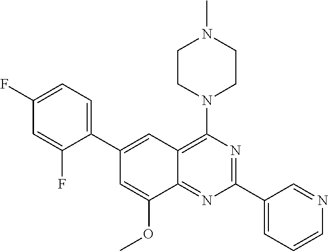 [SMILES: COc1cc(-c2ccc(F)cc2F)cc2c(N3CCN(C)CC3)nc(-c3cccnc3)nc12] |
| PFP-00336 | 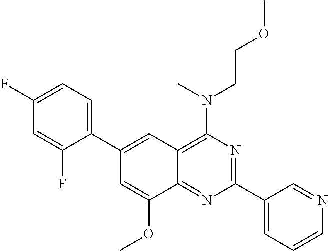 [SMILES: COCCN(C)c1nc(-c2cccnc2)nc2c(OC)cc(-c3ccc(F)cc3F)cc12] |
| PFP-00337 | 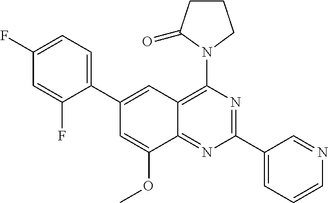 [SMILES: COc1cc(-c2ccc(F)cc2F)cc2c(N3CCCC3=O)nc(-c3cccnc3)nc12] |
| PFP-00338 | 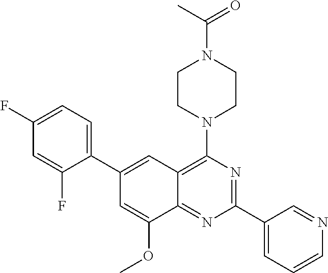 [SMILES: COc1cc(-c2ccc(F)cc2F)cc2c(N3CCN(C(C)=O)CC3)nc(-c3cccnc3)nc12] |
| PFP-00339 | 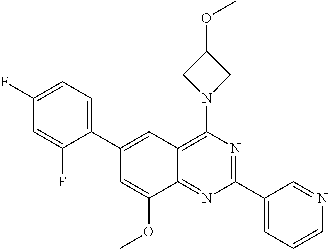 [SMILES: COc1cc(-c2ccc(F)cc2F)cc2c(N3CC(OC)C3)nc(-c3cccnc3)nc12] |
| PFP-00340 | 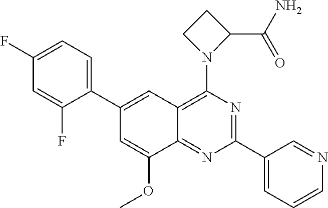 [SMILES: COc1cc(-c2ccc(F)cc2F)cc2c(N3CCC3C(N)=O)nc(-c3cccnc3)nc12] |
| PFP-00341 | 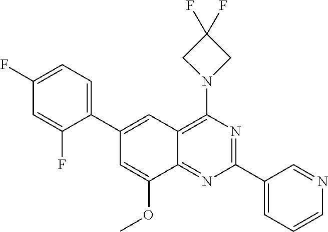 [SMILES: COc1cc(-c2ccc(F)cc2F)cc2c(N3CC(F)(F)C3)nc(-c3cccnc3)nc12] |
| PFP-00342 | 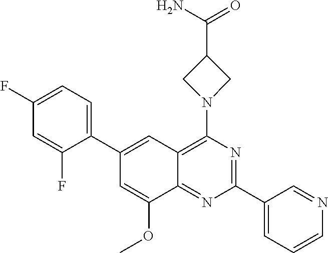 [SMILES: COc1cc(-c2ccc(F)cc2F)cc2c(N3CC(C(N)=O)C3)nc(-c3cccnc3)nc12] |
| PFP-00343 | 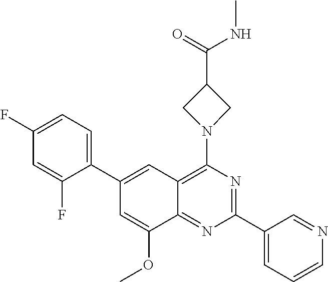 [SMILES: CNC(=O)C1CN(c2nc(-c3cccnc3)nc3c(OC)cc(-c4ccc(F)cc4F)cc23)C1] |
| PFP-00344 | 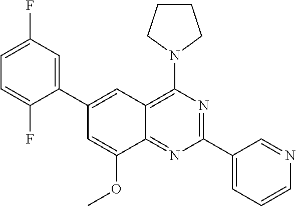 [SMILES: COc1cc(-c2cc(F)ccc2F)cc2c(N3CCCC3)nc(-c3cccnc3)nc12] |
| PFP-00345 | 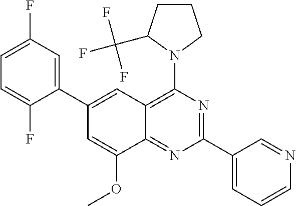 [SMILES: COc1cc(-c2cc(F)ccc2F)cc2c(N3CCCC3C(F)(F)F)nc(-c3cccnc3)nc12] |
| PFP-00346 | 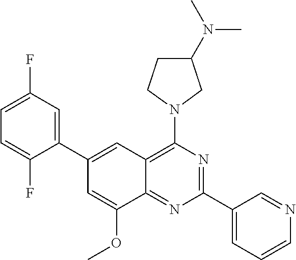 [SMILES: COc1cc(-c2cc(F)ccc2F)cc2c(N3CCC(N(C)C)C3)nc(-c3cccnc3)nc12] |
| PFP-00347 | 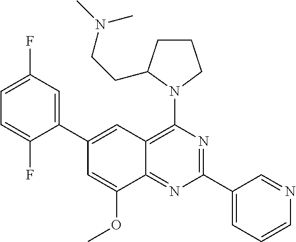 [SMILES: COc1cc(-c2cc(F)ccc2F)cc2c(N3CCCC3CCN(C)C)nc(-c3cccnc3)nc12] |
| PFP-00348 | 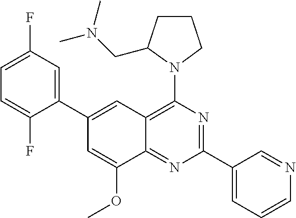 [SMILES: COc1cc(-c2cc(F)ccc2F)cc2c(N3CCCC3CN(C)C)nc(-c3cccnc3)nc12] |
| PFP-00349 | 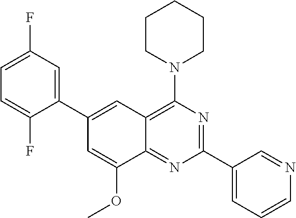 [SMILES: COc1cc(-c2cc(F)ccc2F)cc2c(N3CCCCC3)nc(-c3cccnc3)nc12] |
| PFP-00350 | 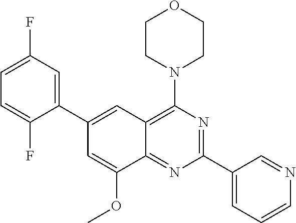 [SMILES: COc1cc(-c2cc(F)ccc2F)cc2c(N3CCOCC3)nc(-c3cccnc3)nc12] |
| PFP-00351 | 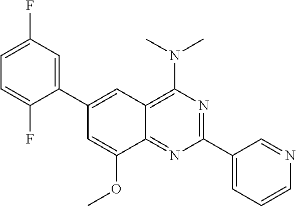 [SMILES: COc1cc(-c2cc(F)ccc2F)cc2c(N(C)C)nc(-c3cccnc3)nc12] |
| PFP-00352 | 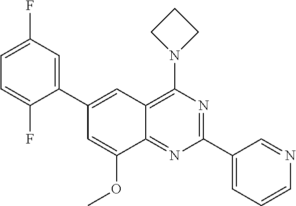 [SMILES: COc1cc(-c2cc(F)ccc2F)cc2c(N3CCC3)nc(-c3cccnc3)nc12] |
| PFP-00353 | 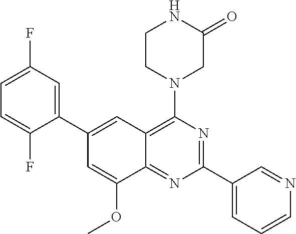 [SMILES: COc1cc(-c2cc(F)ccc2F)cc2c(N3CCNC(=O)C3)nc(-c3cccnc3)nc12] |
| PFP-00354 | 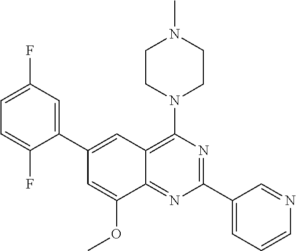 [SMILES: COc1cc(-c2cc(F)ccc2F)cc2c(N3CCN(C)CC3)nc(-c3cccnc3)nc12] |
| PFP-00355 | 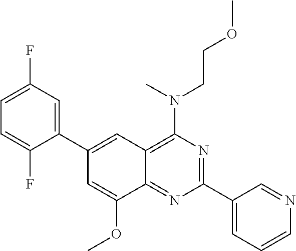 [SMILES: COCCN(C)c1nc(-c2cccnc2)nc2c(OC)cc(-c3cc(F)ccc3F)cc12] |
| PFP-00356 | 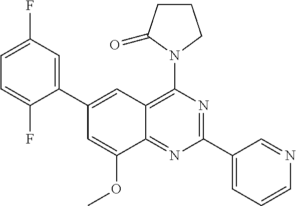 [SMILES: COc1cc(-c2cc(F)ccc2F)cc2c(N3CCCC3=O)nc(-c3cccnc3)nc12] |
| PFP-00357 | 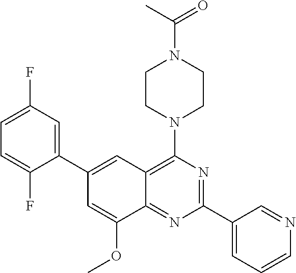 [SMILES: COc1cc(-c2cc(F)ccc2F)cc2c(N3CCN(C(C)=O)CC3)nc(-c3cccnc3)nc12] |
| PFP-00358 | 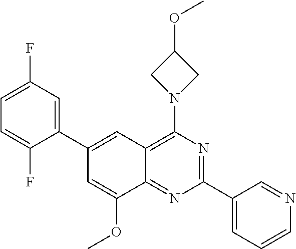 [SMILES: COc1cc(-c2cc(F)ccc2F)cc2c(N3CC(OC)C3)nc(-c3cccnc3)nc12] |
| PFP-00359 | 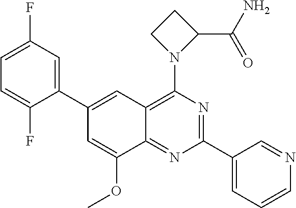 [SMILES: COc1cc(-c2cc(F)ccc2F)cc2c(N3CCC3C(N)=O)nc(-c3cccnc3)nc12] |
| PFP-00360 | 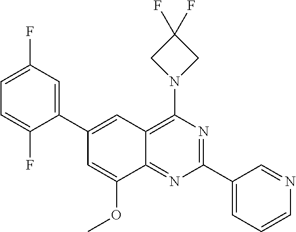 [SMILES: COc1cc(-c2cc(F)ccc2F)cc2c(N3CC(F)(F)C3)nc(-c3cccnc3)nc12] |
| PFP-00361 | 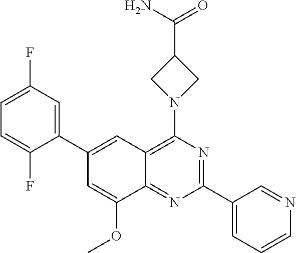 [SMILES: COc1cc(-c2cc(F)ccc2F)cc2c(N3CC(C(N)=O)C3)nc(-c3cccnc3)nc12] |
| PFP-00362 | 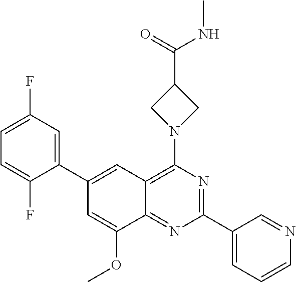 [SMILES: CNC(=O)C1CN(c2nc(-c3cccnc3)nc3c(OC)cc(-c4cc(F)ccc4F)cc23)C1] |
| PFP-00363 | 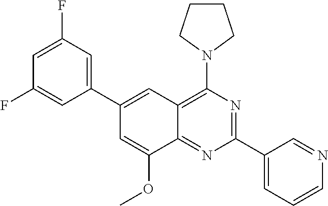 [SMILES: COc1cc(-c2cc(F)cc(F)c2)cc2c(N3CCCC3)nc(-c3cccnc3)nc12] |
| PFP-00364 | 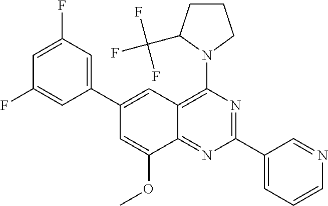 [SMILES: COc1cc(-c2cc(F)cc(F)c2)cc2c(N3CCCC3C(F)(F)F)nc(-c3cccnc3)nc12] |
| PFP-00365 | 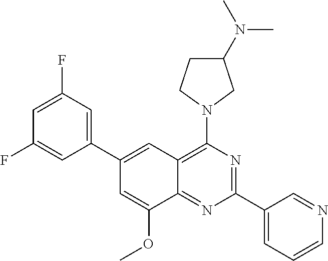 [SMILES: COc1cc(-c2cc(F)cc(F)c2)cc2c(N3CCC(N(C)C)C3)nc(-c3cccnc3)nc12] |
| PFP-00366 | 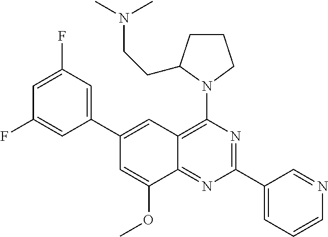 [SMILES: COc1cc(-c2cc(F)cc(F)c2)cc2c(N3CCCC3CCN(C)C)nc(-c3cccnc3)nc12] |
| PFP-00367 | 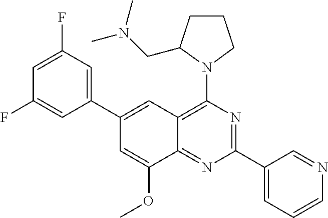 [SMILES: COc1cc(-c2cc(F)cc(F)c2)cc2c(N3CCCC3CN(C)C)nc(-c3cccnc3)nc12] |
| PFP-00368 | 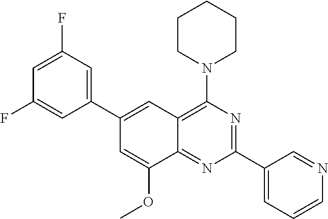 [SMILES: COc1cc(-c2cc(F)cc(F)c2)cc2c(N3CCCCC3)nc(-c3cccnc3)nc12] |
| PFP-00369 | 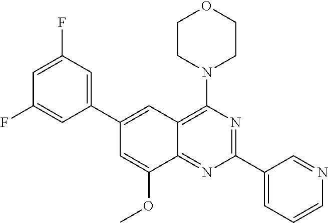 [SMILES: COc1cc(-c2cc(F)cc(F)c2)cc2c(N3CCOCC3)nc(-c3cccnc3)nc12] |
| PFP-00370 | 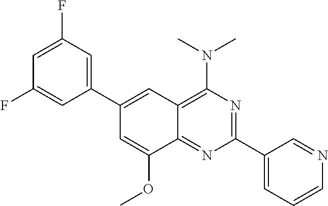 [SMILES: COc1cc(-c2cc(F)cc(F)c2)cc2c(N(C)C)nc(-c3cccnc3)nc12] |
| PFP-00371 | 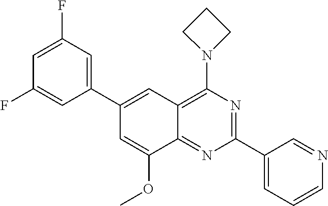 [SMILES: COc1cc(-c2cc(F)cc(F)c2)cc2c(N3CCC3)nc(-c3cccnc3)nc12] |
| PFP-00372 | 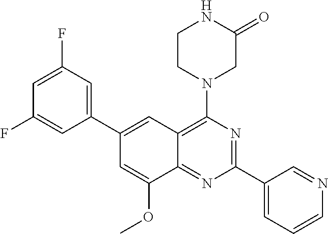 [SMILES: COc1cc(-c2cc(F)cc(F)c2)cc2c(N3CCNC(=O)C3)nc(-c3cccnc3)nc12] |
| PFP-00373 | 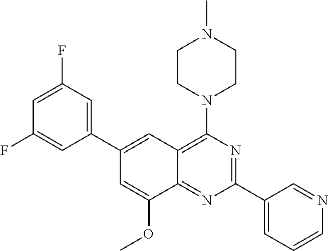 [SMILES: COc1cc(-c2cc(F)cc(F)c2)cc2c(N3CCN(C)CC3)nc(-c3cccnc3)nc12] |
| PFP-00374 | 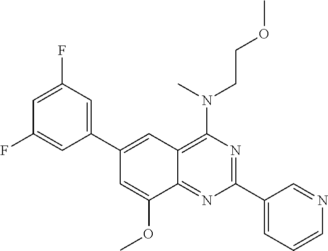 [SMILES: COCCN(C)c1nc(-c2cccnc2)nc2c(OC)cc(-c3cc(F)cc(F)c3)cc12] |
| PFP-00375 | 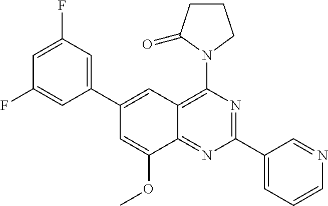 [SMILES: COc1cc(-c2cc(F)cc(F)c2)cc2c(N3CCCC3=O)nc(-c3cccnc3)nc12] |
| PFP-00376 | 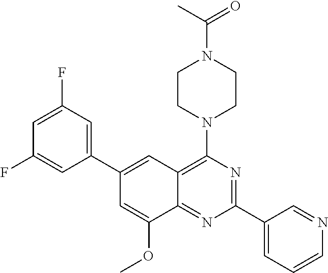 [SMILES: COc1cc(-c2cc(F)cc(F)c2)cc2c(N3CCN(C(C)=O)CC3)nc(-c3cccnc3)nc12] |
| PFP-00377 | 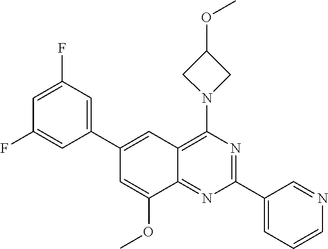 [SMILES: COc1cc(-c2cc(F)cc(F)c2)cc2c(N3CC(OC)C3)nc(-c3cccnc3)nc12] |
| PFP-00378 | 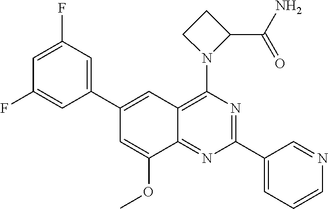 [SMILES: COc1cc(-c2cc(F)cc(F)c2)cc2c(N3CCC3C(N)=O)nc(-c3cccnc3)nc12] |
| PFP-00379 | 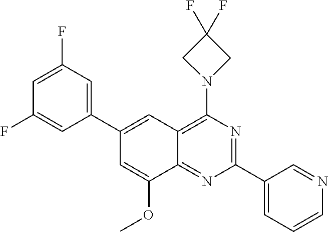 [SMILES: COc1cc(-c2cc(F)cc(F)c2)cc2c(N3CC(F)(F)C3)nc(-c3cccnc3)nc12] |
| PFP-00380 | 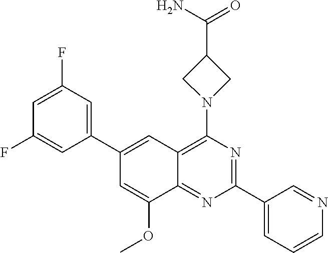 [SMILES: COc1cc(-c2cc(F)cc(F)c2)cc2c(N3CC(C(N)=O)C3)nc(-c3cccnc3)nc12] |
| PFP-00381 | 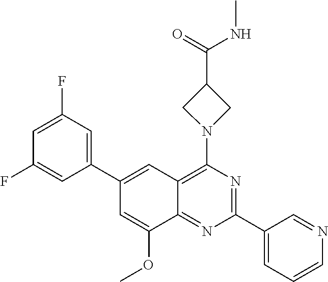 [SMILES: CNC(=O)C1CN(c2nc(-c3cccnc3)nc3c(OC)cc(-c4cc(F)cc(F)c4)cc23)C1] |
| PFP-00382 | 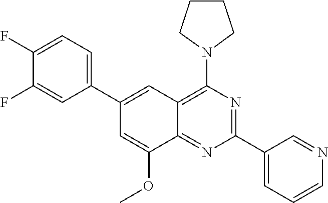 [SMILES: COc1cc(-c2ccc(F)c(F)c2)cc2c(N3CCCC3)nc(-c3cccnc3)nc12] |
| PFP-00383 | 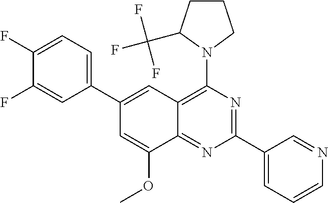 [SMILES: COc1cc(-c2ccc(F)c(F)c2)cc2c(N3CCCC3C(F)(F)F)nc(-c3cccnc3)nc12] |
| PFP-00384 | 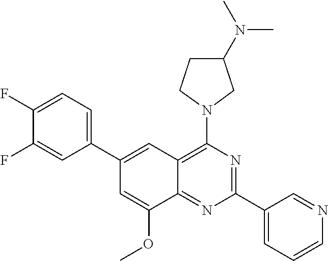 [SMILES: COc1cc(-c2ccc(F)c(F)c2)cc2c(N3CCC(N(C)C)C3)nc(-c3cccnc3)nc12] |
| PFP-00385 | 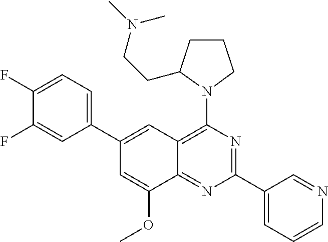 [SMILES: COc1cc(-c2ccc(F)c(F)c2)cc2c(N3CCCC3CCN(C)C)nc(-c3cccnc3)nc12] |
| PFP-00386 | 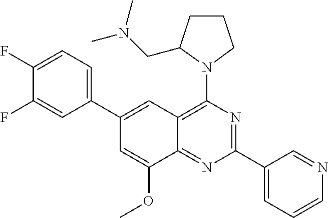 [SMILES: COc1cc(-c2ccc(F)c(F)c2)cc2c(N3CCCC3CN(C)C)nc(-c3cccnc3)nc12] |
| PFP-00387 | 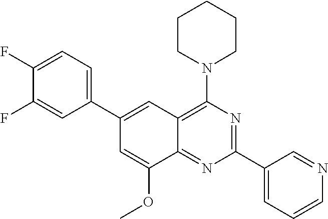 [SMILES: COc1cc(-c2ccc(F)c(F)c2)cc2c(N3CCCCC3)nc(-c3cccnc3)nc12] |
| PFP-00388 | 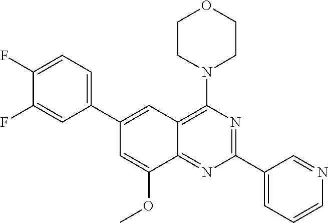 [SMILES: COc1cc(-c2ccc(F)c(F)c2)cc2c(N3CCOCC3)nc(-c3cccnc3)nc12] |
| PFP-00389 | 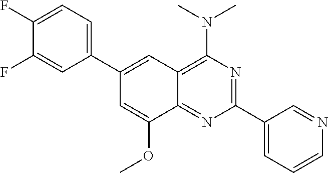 [SMILES: COc1cc(-c2ccc(F)c(F)c2)cc2c(N(C)C)nc(-c3cccnc3)nc12] |
| PFP-00390 | 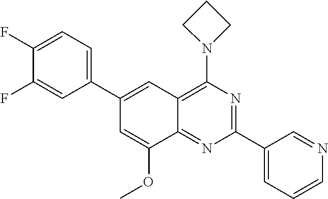 [SMILES: COc1cc(-c2ccc(F)c(F)c2)cc2c(N3CCC3)nc(-c3cccnc3)nc12] |
| PFP-00391 | 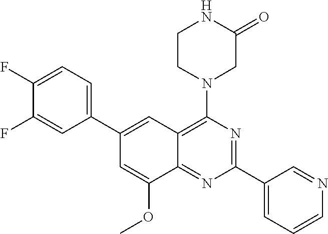 [SMILES: COc1cc(-c2ccc(F)c(F)c2)cc2c(N3CCNC(=O)C3)nc(-c3cccnc3)nc12] |
| PFP-00392 | 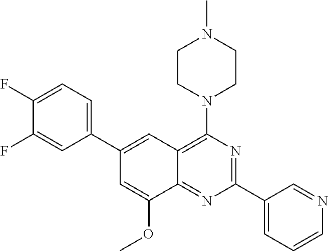 [SMILES: COc1cc(-c2ccc(F)c(F)c2)cc2c(N3CCN(C)CC3)nc(-c3cccnc3)nc12] |
| PFP-00393 | 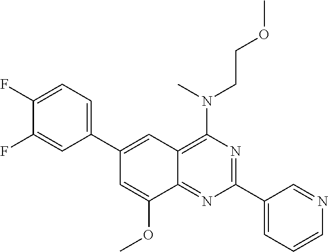 [SMILES: COCCN(C)c1nc(-c2cccnc2)nc2c(OC)cc(-c3ccc(F)c(F)c3)cc12] |
| PFP-00394 | 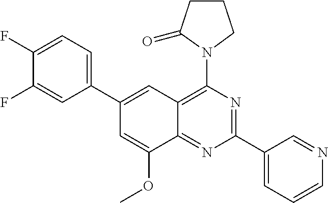 [SMILES: COc1cc(-c2ccc(F)c(F)c2)cc2c(N3CCCC3=O)nc(-c3cccnc3)nc12] |
| PFP-00395 | 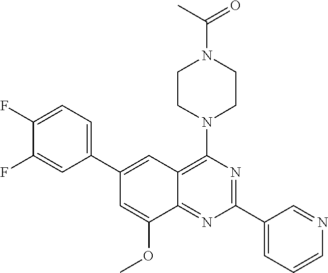 [SMILES: COc1cc(-c2ccc(F)c(F)c2)cc2c(N3CCN(C(C)=O)CC3)nc(-c3cccnc3)nc12] |
| PFP-00396 | 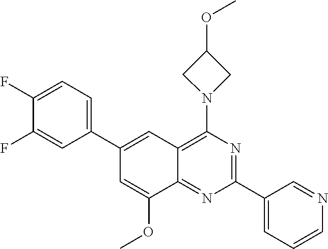 [SMILES: COc1cc(-c2ccc(F)c(F)c2)cc2c(N3CC(OC)C3)nc(-c3cccnc3)nc12] |
| PFP-00397 | 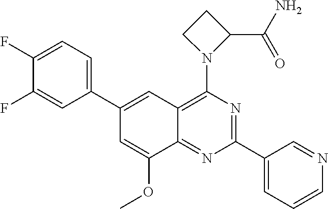 [SMILES: COc1cc(-c2ccc(F)c(F)c2)cc2c(N3CCC3C(N)=O)nc(-c3cccnc3)nc12] |
| PFP-00398 | 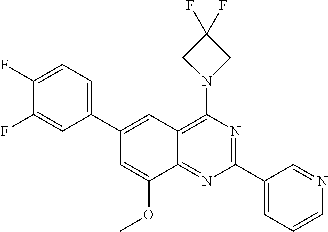 [SMILES: COc1cc(-c2ccc(F)c(F)c2)cc2c(N3CC(F)(F)C3)nc(-c3cccnc3)nc12] |
| PFP-00399 | 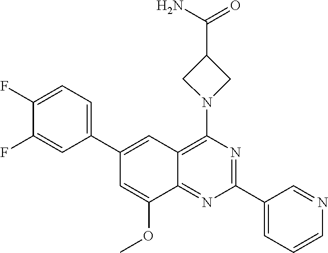 [SMILES: COc1cc(-c2ccc(F)c(F)c2)cc2c(N3CC(C(N)=O)C3)nc(-c3cccnc3)nc12] |
| PFP-00400 | 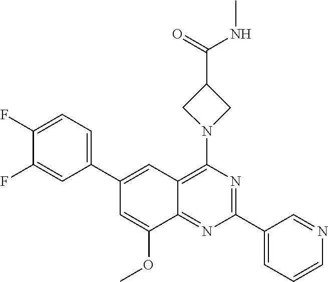 [SMILES: CNC(=O)C1CN(c2nc(-c3cccnc3)nc3c(OC)cc(-c4ccc(F)c(F)c4)cc23)C1] |
| PFP-00419 | 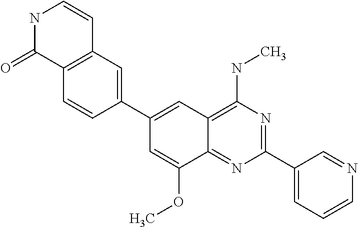 [SMILES: CNc1nc(-c2cccnc2)nc2c(OC)cc(-c3ccc4c(=O)[nH]ccc4c3)cc12] |
| PFP-00420 | 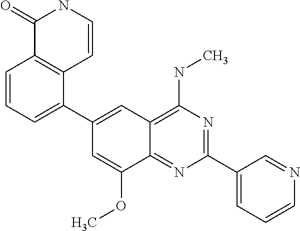 [SMILES: CNc1nc(-c2cccnc2)nc2c(OC)cc(-c3cccc4c(=O)[nH]ccc34)cc12] |
| PFP-00421 | 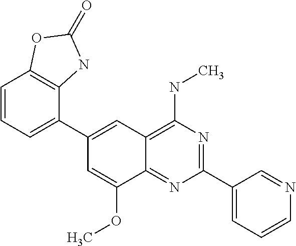 [SMILES: CNc1nc(-c2cccnc2)nc2c(OC)cc(-c3cccc4oc(=O)[nH]c34)cc12] |
| PFP-00422 | 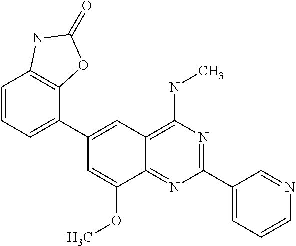 [SMILES: CNc1nc(-c2cccnc2)nc2c(OC)cc(-c3cccc4[nH]c(=O)oc34)cc12] |
| PFP-00423 | 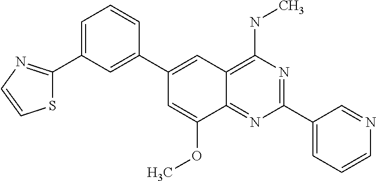 [SMILES: CNc1nc(-c2cccnc2)nc2c(OC)cc(-c3cccc(-c4nccs4)c3)cc12] |
| PFP-00424 | 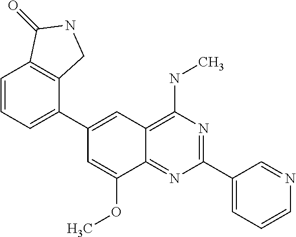 [SMILES: CNc1nc(-c2cccnc2)nc2c(OC)cc(-c3cccc4c3CNC4=O)cc12] |
| PFP-00425 | 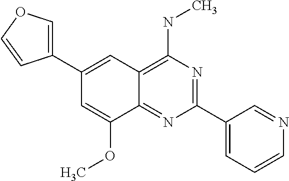 [SMILES: CNc1nc(-c2cccnc2)nc2c(OC)cc(-c3ccoc3)cc12] |
| PFP-00426 | 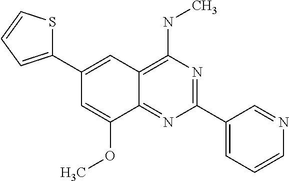 [SMILES: CNc1nc(-c2cccnc2)nc2c(OC)cc(-c3cccs3)cc12] |
| PFP-00427 | 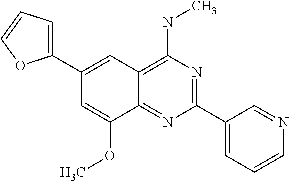 [SMILES: CNc1nc(-c2cccnc2)nc2c(OC)cc(-c3ccco3)cc12] |
| PFP-00428 | 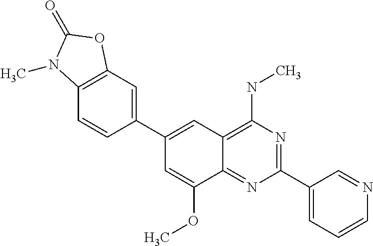 [SMILES: CNc1nc(-c2cccnc2)nc2c(OC)cc(-c3ccc4c(c3)oc(=O)n4C)cc12] |
| PFP-00429 | 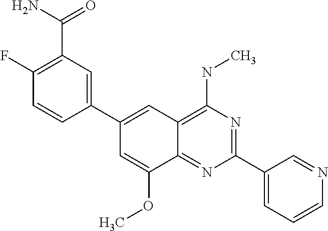 [SMILES: CNc1nc(-c2cccnc2)nc2c(OC)cc(-c3ccc(F)c(C(N)=O)c3)cc12] |
| PFP-00430 | 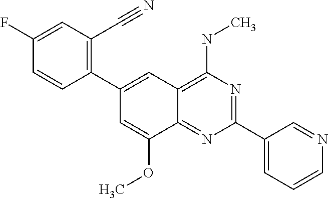 [SMILES: CNc1nc(-c2cccnc2)nc2c(OC)cc(-c3ccc(F)cc3C#N)cc12] |
| PFP-00431 | 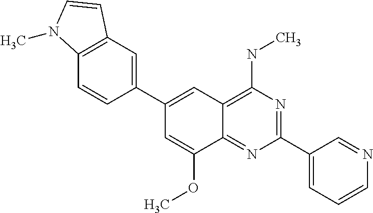 [SMILES: CNc1nc(-c2cccnc2)nc2c(OC)cc(-c3ccc4c(ccn4C)c3)cc12] |
| PFP-00432 | 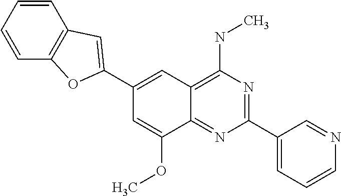 [SMILES: CNc1nc(-c2cccnc2)nc2c(OC)cc(-c3cc4ccccc4o3)cc12] |
| PFP-00433 | 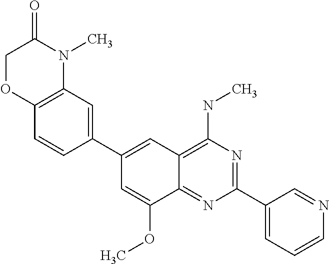 [SMILES: CNc1nc(-c2cccnc2)nc2c(OC)cc(-c3ccc4c(c3)N(C)C(=O)CO4)cc12] |
| PFP-00434 | 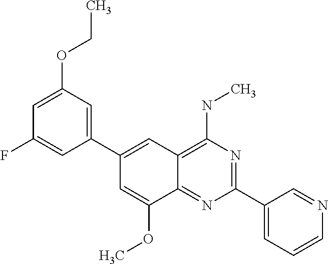 [SMILES: CCOc1cc(F)cc(-c2cc(OC)c3nc(-c4cccnc4)nc(NC)c3c2)c1] |
| PFP-00435 | 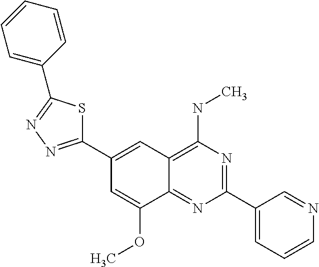 [SMILES: CNc1nc(-c2cccnc2)nc2c(OC)cc(-c3nnc(-c4ccccc4)s3)cc12] |
| PFP-00436 | 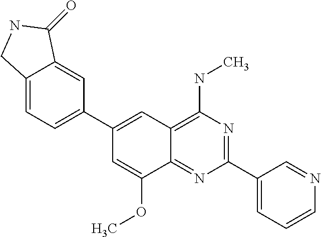 [SMILES: CNc1nc(-c2cccnc2)nc2c(OC)cc(-c3ccc4c(c3)C(=O)NC4)cc12] |
| PFP-00437 | 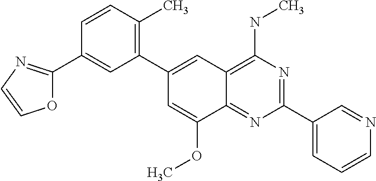 [SMILES: CNc1nc(-c2cccnc2)nc2c(OC)cc(-c3cc(-c4ncco4)ccc3C)cc12] |
| PFP-00438 | 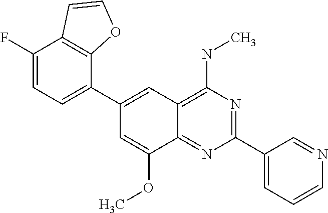 [SMILES: CNc1nc(-c2cccnc2)nc2c(OC)cc(-c3ccc(F)c4ccoc34)cc12] |
| PFP-00439 | 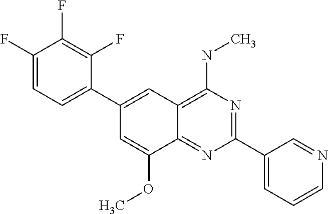 [SMILES: CNc1nc(-c2cccnc2)nc2c(OC)cc(-c3ccc(F)c(F)c3F)cc12] |
| PFP-00440 | 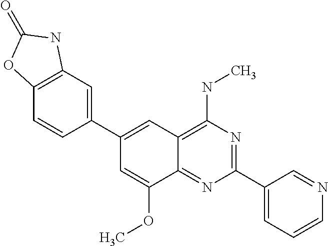 [SMILES: CNc1nc(-c2cccnc2)nc2c(OC)cc(-c3ccc4oc(=O)[nH]c4c3)cc12] |
| PFP-00441 | 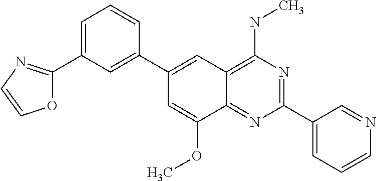 [SMILES: CNc1nc(-c2cccnc2)nc2c(OC)cc(-c3cccc(-c4ncco4)c3)cc12] |
| PFP-00442 | 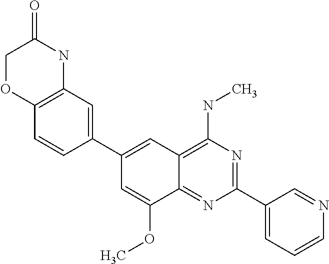 [SMILES: CNc1nc(-c2cccnc2)nc2c(OC)cc(-c3ccc4c(c3)NC(=O)CO4)cc12] |
| PFP-00443 | 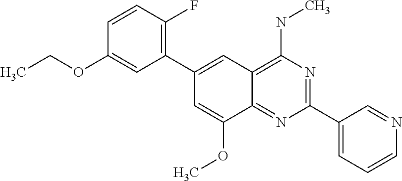 [SMILES: CCOc1ccc(F)c(-c2cc(OC)c3nc(-c4cccnc4)nc(NC)c3c2)c1] |
| PFP-00444 | 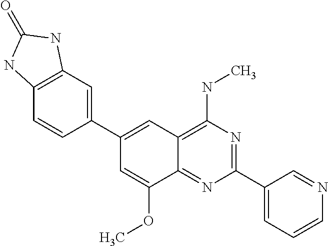 [SMILES: CNc1nc(-c2cccnc2)nc2c(OC)cc(-c3ccc4[nH]c(=O)[nH]c4c3)cc12] |
| PFP-00445 | 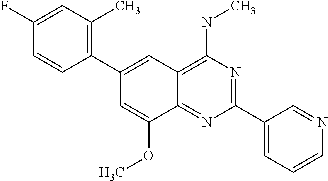 [SMILES: CNc1nc(-c2cccnc2)nc2c(OC)cc(-c3ccc(F)cc3C)cc12] |
| PFP-00446 | 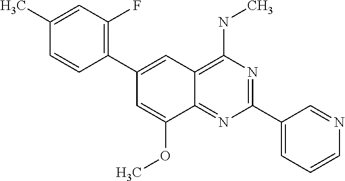 [SMILES: CNc1nc(-c2cccnc2)nc2c(OC)cc(-c3ccc(C)cc3F)cc12] |
| PFP-00447 | 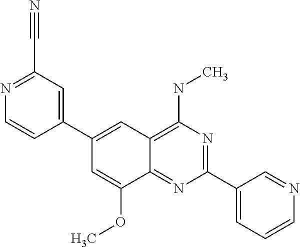 [SMILES: CNc1nc(-c2cccnc2)nc2c(OC)cc(-c3ccnc(C#N)c3)cc12] |
| PFP-00448 | 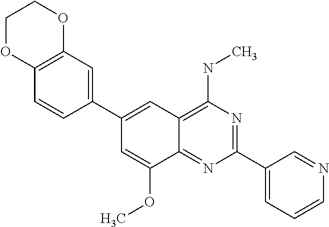 [SMILES: CNc1nc(-c2cccnc2)nc2c(OC)cc(-c3ccc4c(c3)OCCO4)cc12] |
| PFP-00449 | 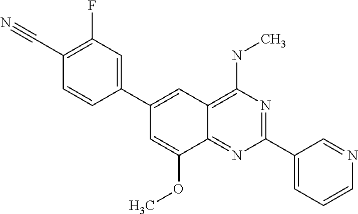 [SMILES: CNc1nc(-c2cccnc2)nc2c(OC)cc(-c3ccc(C#N)c(F)c3)cc12] |
| PFP-00450 | 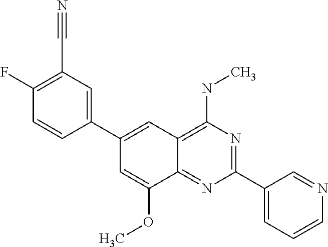 [SMILES: CNc1nc(-c2cccnc2)nc2c(OC)cc(-c3ccc(F)c(C#N)c3)cc12] |
| PFP-00451 | 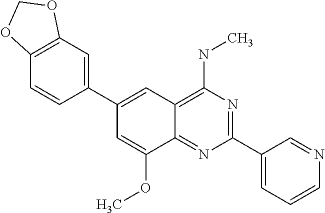 [SMILES: CNc1nc(-c2cccnc2)nc2c(OC)cc(-c3ccc4c(c3)OCO4)cc12] |
| PFP-00452 | 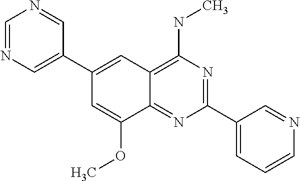 [SMILES: CNc1nc(-c2cccnc2)nc2c(OC)cc(-c3cncnc3)cc12] |
| PFP-00453 | 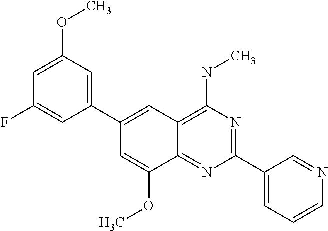 [SMILES: CNc1nc(-c2cccnc2)nc2c(OC)cc(-c3cc(F)cc(OC)c3)cc12] |
| PFP-00454 | 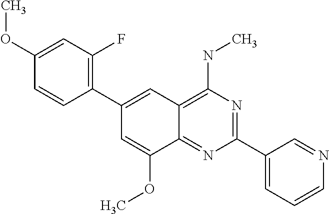 [SMILES: CNc1nc(-c2cccnc2)nc2c(OC)cc(-c3ccc(OC)cc3F)cc12] |
| PFP-00455 | 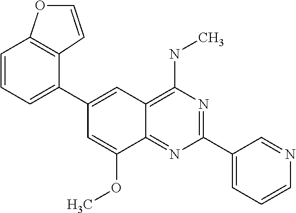 [SMILES: CNc1nc(-c2cccnc2)nc2c(OC)cc(-c3cccc4occc34)cc12] |
| PFP-00456 | 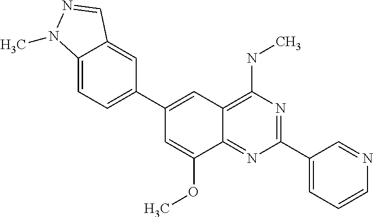 [SMILES: CNc1nc(-c2cccnc2)nc2c(OC)cc(-c3ccc4c(cnn4C)c3)cc12] |
| PFP-00457 | 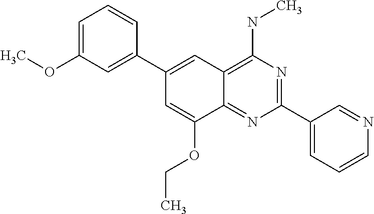 [SMILES: CCOc1cc(-c2cccc(OC)c2)cc2c(NC)nc(-c3cccnc3)nc12] |
| PFP-00458 | 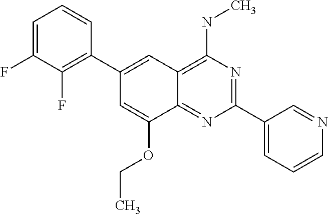 [SMILES: CCOc1cc(-c2cccc(F)c2F)cc2c(NC)nc(-c3cccnc3)nc12] |
| PFP-00459 | 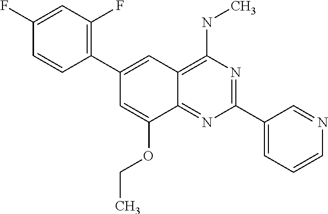 [SMILES: CCOc1cc(-c2ccc(F)cc2F)cc2c(NC)nc(-c3cccnc3)nc12] |
| PFP-00460 | 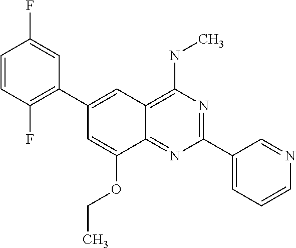 [SMILES: CCOc1cc(-c2cc(F)ccc2F)cc2c(NC)nc(-c3cccnc3)nc12] |
| PFP-00461 | 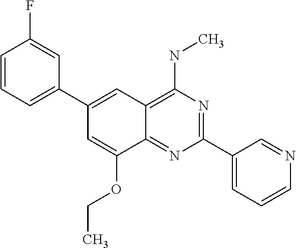 [SMILES: CCOc1cc(-c2cccc(F)c2)cc2c(NC)nc(-c3cccnc3)nc12] |
| PFP-00462 | 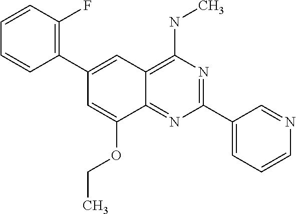 [SMILES: CCOc1cc(-c2ccccc2F)cc2c(NC)nc(-c3cccnc3)nc12] |
| PFP-00463 | 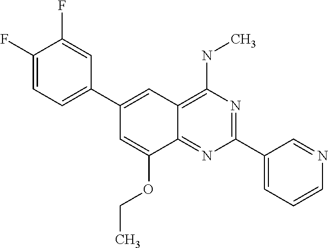 [SMILES: CCOc1cc(-c2ccc(F)c(F)c2)cc2c(NC)nc(-c3cccnc3)nc12] |
| PFP-00464 | 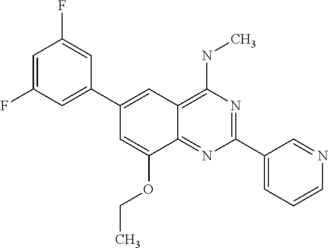 [SMILES: CCOc1cc(-c2cc(F)cc(F)c2)cc2c(NC)nc(-c3cccnc3)nc12] |
| PFP-00465 | 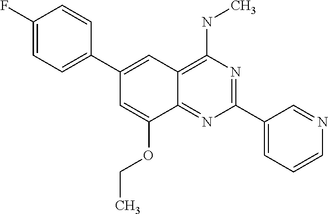 [SMILES: CCOc1cc(-c2ccc(F)cc2)cc2c(NC)nc(-c3cccnc3)nc12] |
| PFP-00466 | 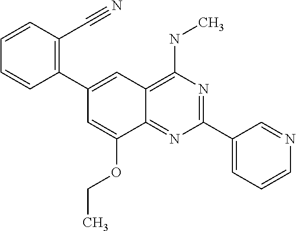 [SMILES: CCOc1cc(-c2ccccc2C#N)cc2c(NC)nc(-c3cccnc3)nc12] |
| PFP-00467 | 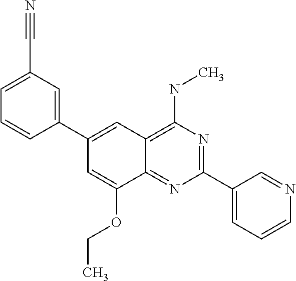 [SMILES: CCOc1cc(-c2cccc(C#N)c2)cc2c(NC)nc(-c3cccnc3)nc12] |
| PFP-00468 | 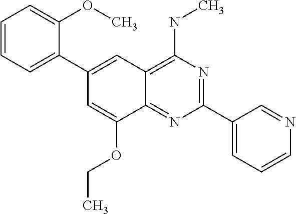 [SMILES: CCOc1cc(-c2ccccc2OC)cc2c(NC)nc(-c3cccnc3)nc12] |
| PFP-00469 | 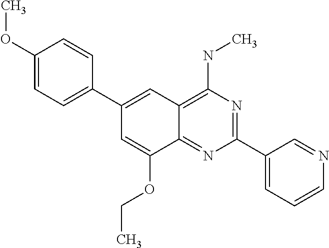 [SMILES: CCOc1cc(-c2ccc(OC)cc2)cc2c(NC)nc(-c3cccnc3)nc12] |
| PFP-00470 | 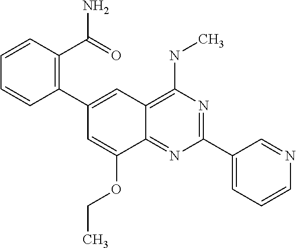 [SMILES: CCOc1cc(-c2ccccc2C(N)=O)cc2c(NC)nc(-c3cccnc3)nc12] |
| PFP-00471 | 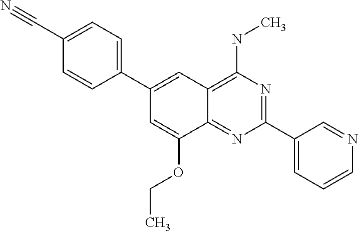 [SMILES: CCOc1cc(-c2ccc(C#N)cc2)cc2c(NC)nc(-c3cccnc3)nc12] |
| PFP-00472 | 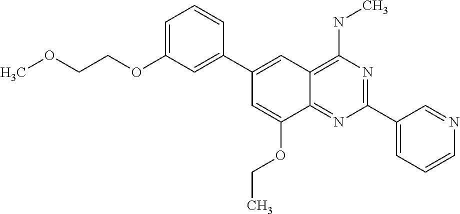 [SMILES: CCOc1cc(-c2cccc(OCCOC)c2)cc2c(NC)nc(-c3cccnc3)nc12] |
| PFP-00473 | 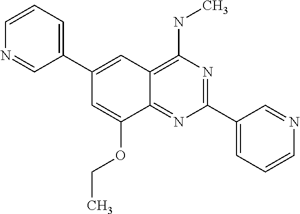 [SMILES: CCOc1cc(-c2cccnc2)cc2c(NC)nc(-c3cccnc3)nc12] |
| PFP-00474 | 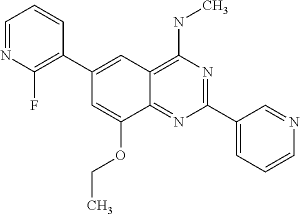 [SMILES: CCOc1cc(-c2cccnc2F)cc2c(NC)nc(-c3cccnc3)nc12] |
| PFP-00475 | 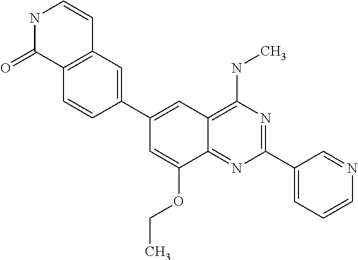 [SMILES: CCOc1cc(-c2ccc3c(=O)[nH]ccc3c2)cc2c(NC)nc(-c3cccnc3)nc12] |
| PFP-00476 | 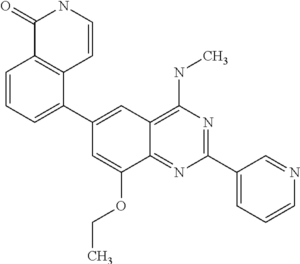 [SMILES: CCOc1cc(-c2cccc3c(=O)[nH]ccc23)cc2c(NC)nc(-c3cccnc3)nc12] |
| PFP-00477 | 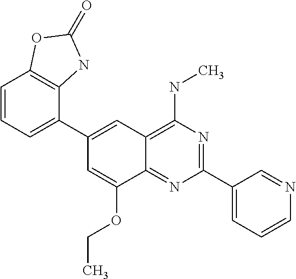 [SMILES: CCOc1cc(-c2cccc3oc(=O)[nH]c23)cc2c(NC)nc(-c3cccnc3)nc12] |
| PFP-00478 | 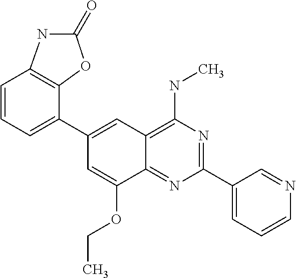 [SMILES: CCOc1cc(-c2cccc3[nH]c(=O)oc23)cc2c(NC)nc(-c3cccnc3)nc12] |
| PFP-00479 | 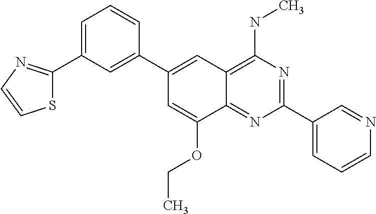 [SMILES: CCOc1cc(-c2cccc(-c3nccs3)c2)cc2c(NC)nc(-c3cccnc3)nc12] |
| PFP-00480 | 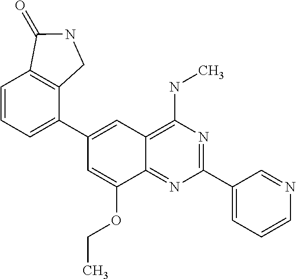 [SMILES: CCOc1cc(-c2cccc3c2CNC3=O)cc2c(NC)nc(-c3cccnc3)nc12] |
| PFP-00481 | 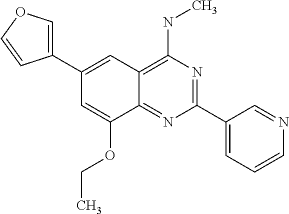 [SMILES: CCOc1cc(-c2ccoc2)cc2c(NC)nc(-c3cccnc3)nc12] |
| PFP-00482 | 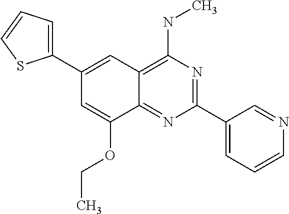 [SMILES: CCOc1cc(-c2cccs2)cc2c(NC)nc(-c3cccnc3)nc12] |
| PFP-00483 | 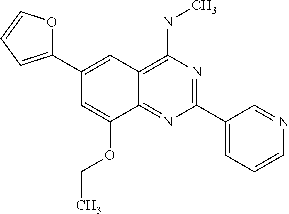 [SMILES: CCOc1cc(-c2ccco2)cc2c(NC)nc(-c3cccnc3)nc12] |
| PFP-00484 | 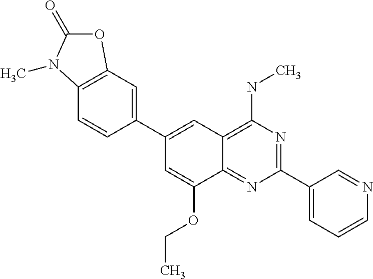 [SMILES: CCOc1cc(-c2ccc3c(c2)oc(=O)n3C)cc2c(NC)nc(-c3cccnc3)nc12] |
| PFP-00485 | 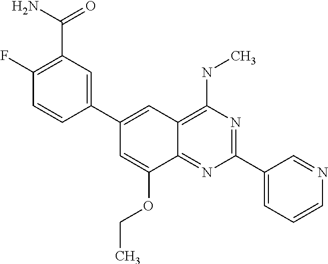 [SMILES: CCOc1cc(-c2ccc(F)c(C(N)=O)c2)cc2c(NC)nc(-c3cccnc3)nc12] |
| PFP-00486 | 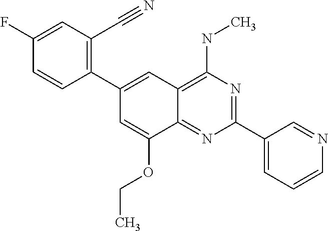 [SMILES: CCOc1cc(-c2ccc(F)cc2C#N)cc2c(NC)nc(-c3cccnc3)nc12] |
| PFP-00487 | 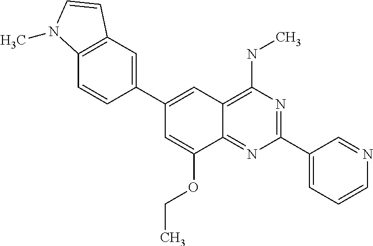 [SMILES: CCOc1cc(-c2ccc3c(ccn3C)c2)cc2c(NC)nc(-c3cccnc3)nc12] |
| PFP-00488 | 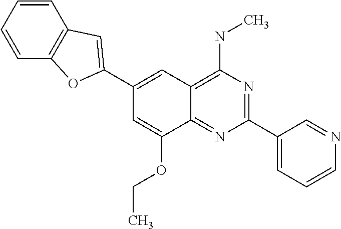 [SMILES: CCOc1cc(-c2cc3ccccc3o2)cc2c(NC)nc(-c3cccnc3)nc12] |
| PFP-00489 | 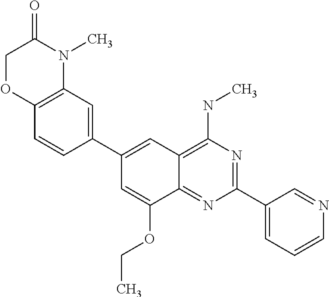 [SMILES: CCOc1cc(-c2ccc3c(c2)N(C)C(=O)CO3)cc2c(NC)nc(-c3cccnc3)nc12] |
| PFP-00490 | 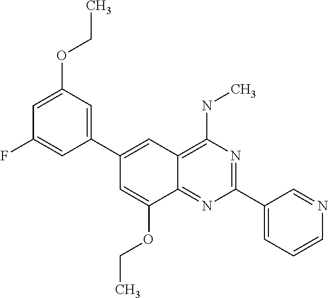 [SMILES: CCOc1cc(F)cc(-c2cc(OCC)c3nc(-c4cccnc4)nc(NC)c3c2)c1] |
| PFP-00491 | 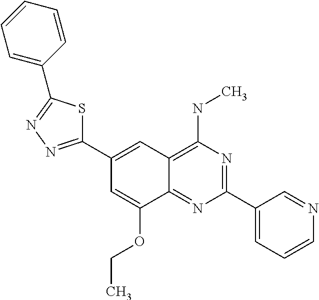 [SMILES: CCOc1cc(-c2nnc(-c3ccccc3)s2)cc2c(NC)nc(-c3cccnc3)nc12] |
| PFP-00492 | 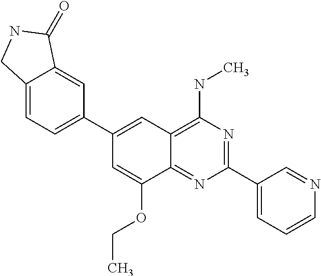 [SMILES: CCOc1cc(-c2ccc3c(c2)C(=O)NC3)cc2c(NC)nc(-c3cccnc3)nc12] |
| PFP-00493 | 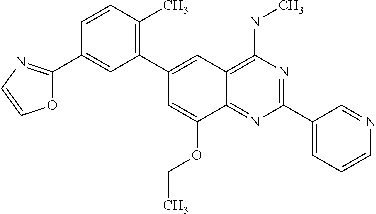 [SMILES: CCOc1cc(-c2cc(-c3ncco3)ccc2C)cc2c(NC)nc(-c3cccnc3)nc12] |
| PFP-00494 | 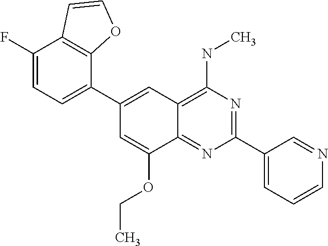 [SMILES: CCOc1cc(-c2ccc(F)c3ccoc23)cc2c(NC)nc(-c3cccnc3)nc12] |
| PFP-00495 | 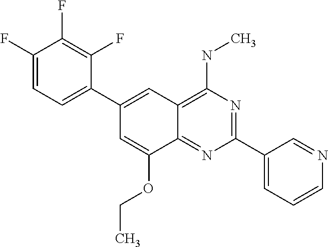 [SMILES: CCOc1cc(-c2ccc(F)c(F)c2F)cc2c(NC)nc(-c3cccnc3)nc12] |
| PFP-00496 | 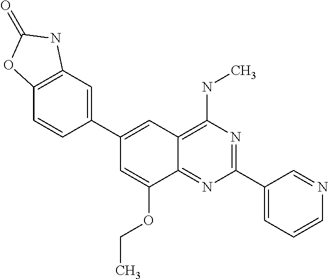 [SMILES: CCOc1cc(-c2ccc3oc(=O)[nH]c3c2)cc2c(NC)nc(-c3cccnc3)nc12] |
| PFP-00497 | 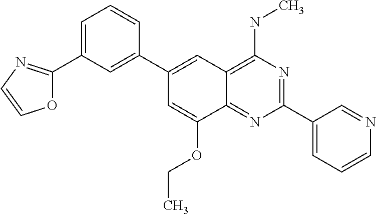 [SMILES: CCOc1cc(-c2cccc(-c3ncco3)c2)cc2c(NC)nc(-c3cccnc3)nc12] |
| PFP-00498 | 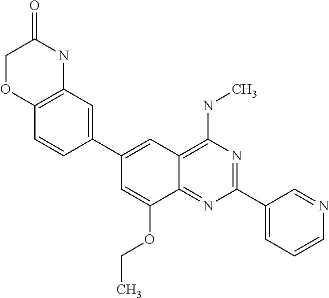 [SMILES: CCOc1cc(-c2ccc3c(c2)NC(=O)CO3)cc2c(NC)nc(-c3cccnc3)nc12] |
| PFP-00499 | 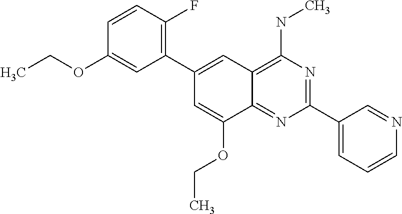 [SMILES: CCOc1ccc(F)c(-c2cc(OCC)c3nc(-c4cccnc4)nc(NC)c3c2)c1] |
| PFP-00500 | 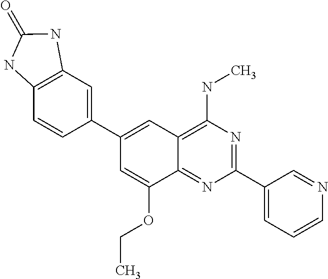 [SMILES: CCOc1cc(-c2ccc3[nH]c(=O)[nH]c3c2)cc2c(NC)nc(-c3cccnc3)nc12] |
| PFP-00501 | 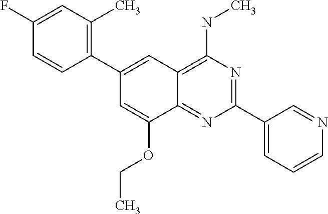 [SMILES: CCOc1cc(-c2ccc(F)cc2C)cc2c(NC)nc(-c3cccnc3)nc12] |
| PFP-00502 | 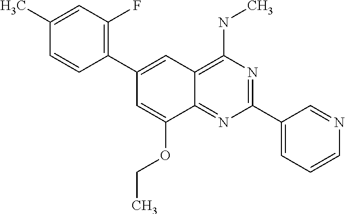 [SMILES: CCOc1cc(-c2ccc(C)cc2F)cc2c(NC)nc(-c3cccnc3)nc12] |
| PFP-00503 | 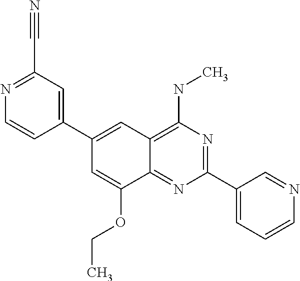 [SMILES: CCOc1cc(-c2ccnc(C#N)c2)cc2c(NC)nc(-c3cccnc3)nc12] |
| PFP-00504 | 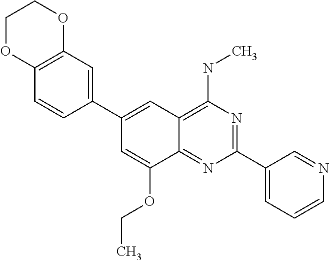 [SMILES: CCOc1cc(-c2ccc3c(c2)OCCO3)cc2c(NC)nc(-c3cccnc3)nc12] |
| PFP-00505 | 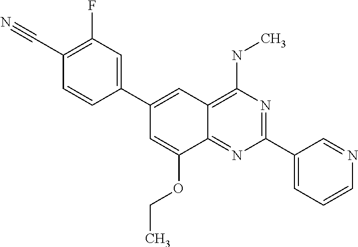 [SMILES: CCOc1cc(-c2ccc(C#N)c(F)c2)cc2c(NC)nc(-c3cccnc3)nc12] |
| PFP-00506 | 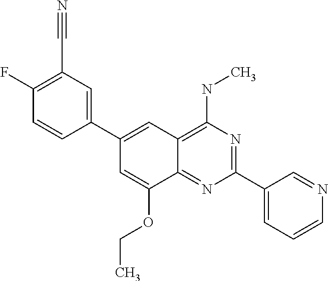 [SMILES: CCOc1cc(-c2ccc(F)c(C#N)c2)cc2c(NC)nc(-c3cccnc3)nc12] |
| PFP-00507 | 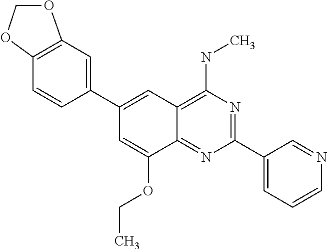 [SMILES: CCOc1cc(-c2ccc3c(c2)OCO3)cc2c(NC)nc(-c3cccnc3)nc12] |
| PFP-00508 | 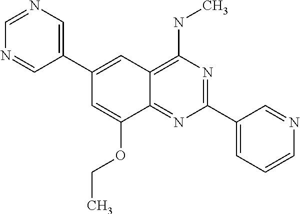 [SMILES: CCOc1cc(-c2cncnc2)cc2c(NC)nc(-c3cccnc3)nc12] |
| PFP-00509 | 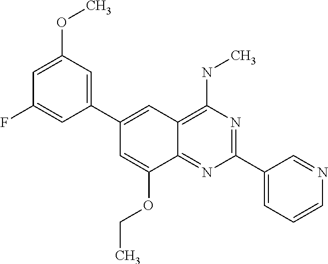 [SMILES: CCOc1cc(-c2cc(F)cc(OC)c2)cc2c(NC)nc(-c3cccnc3)nc12] |
| PFP-00510 | 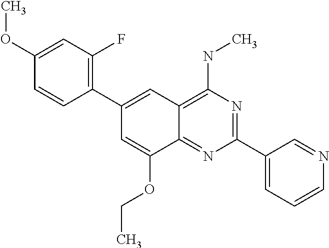 [SMILES: CCOc1cc(-c2ccc(OC)cc2F)cc2c(NC)nc(-c3cccnc3)nc12] |
| PFP-00511 | 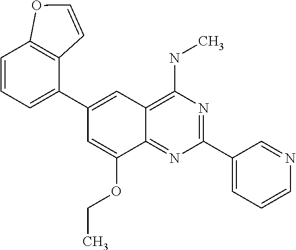 [SMILES: CCOc1cc(-c2cccc3occc23)cc2c(NC)nc(-c3cccnc3)nc12] |
| PFP-00512 | 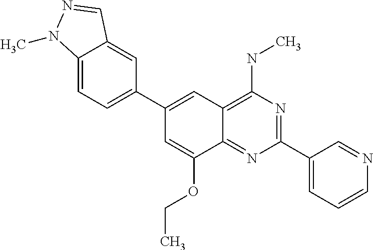 [SMILES: CCOc1cc(-c2ccc3c(cnn3C)c2)cc2c(NC)nc(-c3cccnc3)nc12] |
| PFP-00515 | 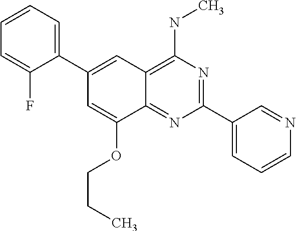 [SMILES: CCCOc1cc(-c2ccccc2F)cc2c(NC)nc(-c3cccnc3)nc12] |
| PFP-00516 | 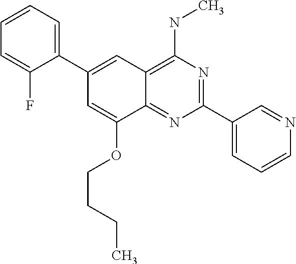 [SMILES: CCCCOc1cc(-c2ccccc2F)cc2c(NC)nc(-c3cccnc3)nc12] |
| PFP-00517 | 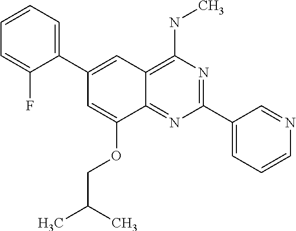 [SMILES: CNc1nc(-c2cccnc2)nc2c(OCC(C)C)cc(-c3ccccc3F)cc12] |
| PFP-00518 | 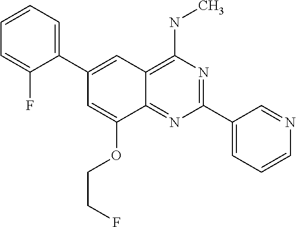 [SMILES: CNc1nc(-c2cccnc2)nc2c(OCCF)cc(-c3ccccc3F)cc12] |
| PFP-00519 | 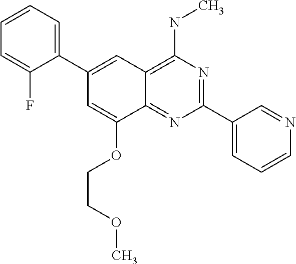 [SMILES: CNc1nc(-c2cccnc2)nc2c(OCCOC)cc(-c3ccccc3F)cc12] |
| PFP-00520 | 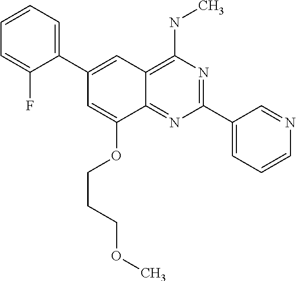 [SMILES: CNc1nc(-c2cccnc2)nc2c(OCCCOC)cc(-c3ccccc3F)cc12] |
| PFP-00521 | 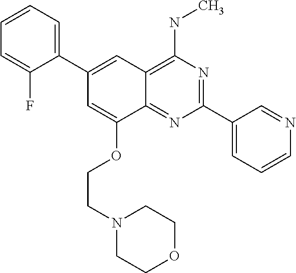 [SMILES: CNc1nc(-c2cccnc2)nc2c(OCCN3CCOCC3)cc(-c3ccccc3F)cc12] |
| PFP-00522 | 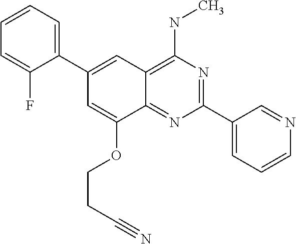 [SMILES: CNc1nc(-c2cccnc2)nc2c(OCCC#N)cc(-c3ccccc3F)cc12] |
| PFP-00523 | 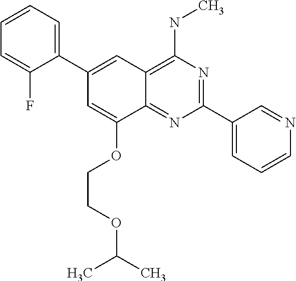 [SMILES: CNc1nc(-c2cccnc2)nc2c(OCCOC(C)C)cc(-c3ccccc3F)cc12] |
| PFP-00524 | 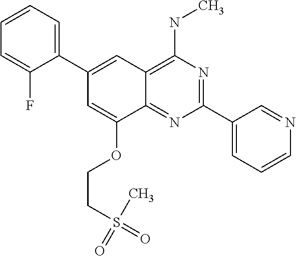 [SMILES: CNc1nc(-c2cccnc2)nc2c(OCCS(C)(=O)=O)cc(-c3ccccc3F)cc12] |
| PFP-00525 | 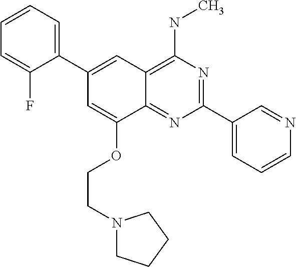 [SMILES: CNc1nc(-c2cccnc2)nc2c(OCCN3CCCC3)cc(-c3ccccc3F)cc12] |
| PFP-00526 | 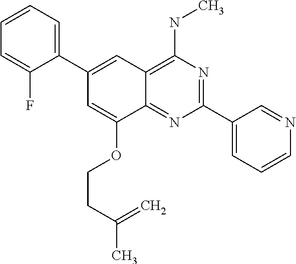 [SMILES: C=C(C)CCOc1cc(-c2ccccc2F)cc2c(NC)nc(-c3cccnc3)nc12] |
| PFP-00527 | 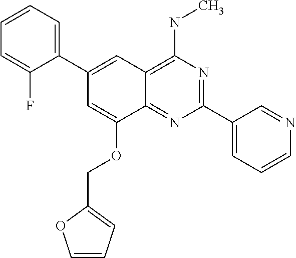 [SMILES: CNc1nc(-c2cccnc2)nc2c(OCc3ccco3)cc(-c3ccccc3F)cc12] |
| PFP-00528 | 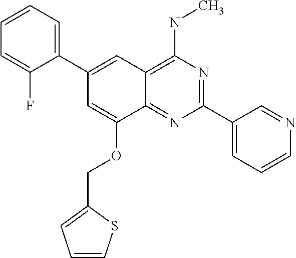 [SMILES: CNc1nc(-c2cccnc2)nc2c(OCc3cccs3)cc(-c3ccccc3F)cc12] |
| PFP-00529 | 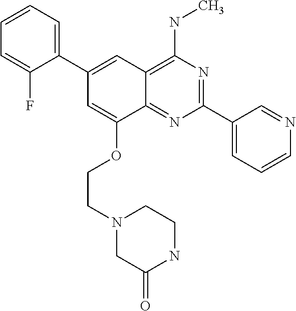 [SMILES: CNc1nc(-c2cccnc2)nc2c(OCCN3CCNC(=O)C3)cc(-c3ccccc3F)cc12] |
| PFP-00530 | 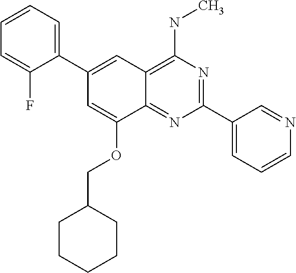 [SMILES: CNc1nc(-c2cccnc2)nc2c(OCC3CCCCC3)cc(-c3ccccc3F)cc12] |
| PFP-00531 | 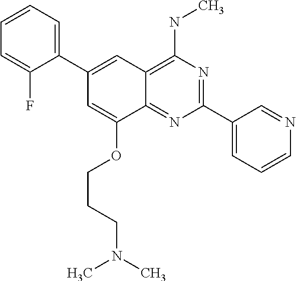 [SMILES: CNc1nc(-c2cccnc2)nc2c(OCCCN(C)C)cc(-c3ccccc3F)cc12] |
| PFP-00532 | 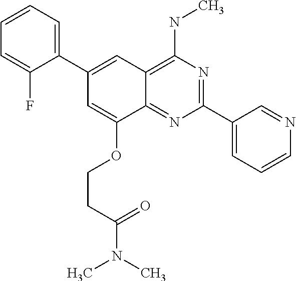 [SMILES: CNc1nc(-c2cccnc2)nc2c(OCCC(=O)N(C)C)cc(-c3ccccc3F)cc12] |
| PFP-00533 | 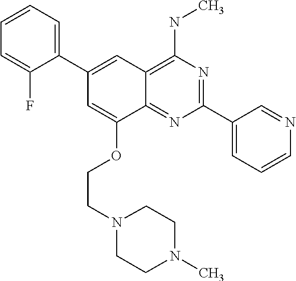 [SMILES: CNc1nc(-c2cccnc2)nc2c(OCCN3CCN(C)CC3)cc(-c3ccccc3F)cc12] |
| PFP-00534 | 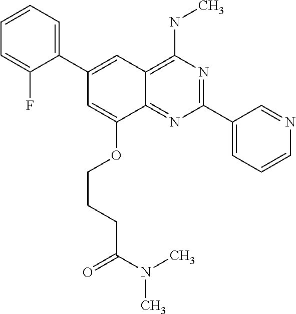 [SMILES: CNc1nc(-c2cccnc2)nc2c(OCCCC(=O)N(C)C)cc(-c3ccccc3F)cc12] |
| PFP-00535 | 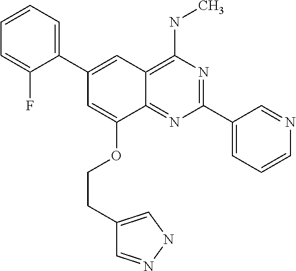 [SMILES: CNc1nc(-c2cccnc2)nc2c(OCCc3cn[nH]c3)cc(-c3ccccc3F)cc12] |
| PFP-00536 | 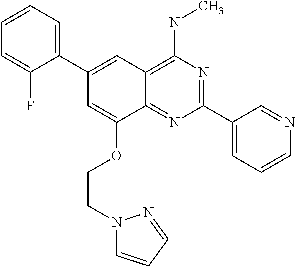 [SMILES: CNc1nc(-c2cccnc2)nc2c(OCCn3cccn3)cc(-c3ccccc3F)cc12] |
| PFP-00537 | 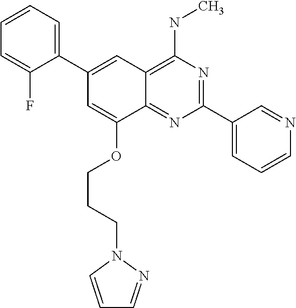 [SMILES: CNc1nc(-c2cccnc2)nc2c(OCCCn3cccn3)cc(-c3ccccc3F)cc12] |
| PFP-00538 | 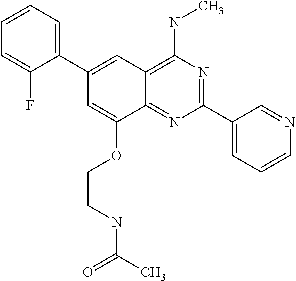 [SMILES: CNc1nc(-c2cccnc2)nc2c(OCCNC(C)=O)cc(-c3ccccc3F)cc12] |
| PFP-00539 | 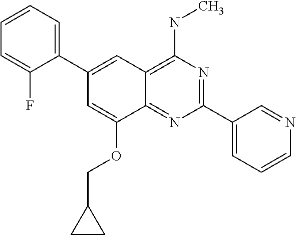 [SMILES: CNc1nc(-c2cccnc2)nc2c(OCC3CC3)cc(-c3ccccc3F)cc12] |
| PFP-00540 | 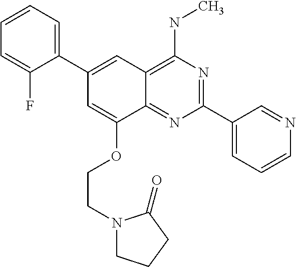 [SMILES: CNc1nc(-c2cccnc2)nc2c(OCCN3CCCC3=O)cc(-c3ccccc3F)cc12] |
| PFP-00541 | 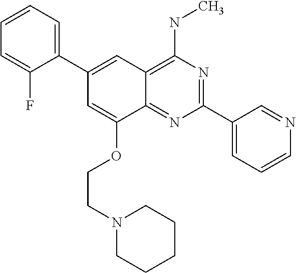 [SMILES: CNc1nc(-c2cccnc2)nc2c(OCCN3CCCCC3)cc(-c3ccccc3F)cc12] |
| PFP-00542 | 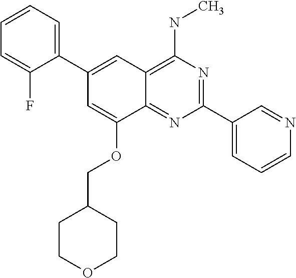 [SMILES: CNc1nc(-c2cccnc2)nc2c(OCC3CCOCC3)cc(-c3ccccc3F)cc12] |
| PFP-00545 | 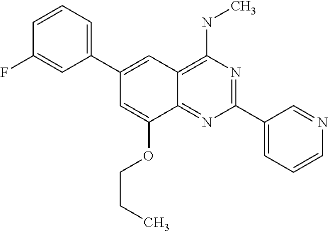 [SMILES: CCCOc1cc(-c2cccc(F)c2)cc2c(NC)nc(-c3cccnc3)nc12] |
| PFP-00546 | 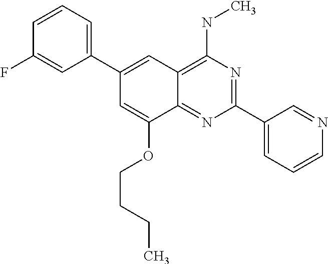 [SMILES: CCCCOc1cc(-c2cccc(F)c2)cc2c(NC)nc(-c3cccnc3)nc12] |
| PFP-00547 | 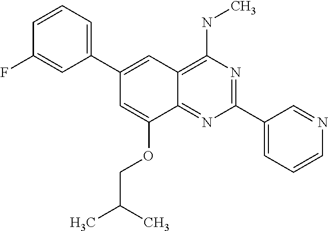 [SMILES: CNc1nc(-c2cccnc2)nc2c(OCC(C)C)cc(-c3cccc(F)c3)cc12] |
| PFP-00548 | 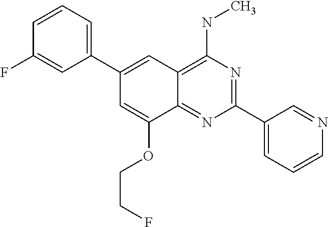 [SMILES: CNc1nc(-c2cccnc2)nc2c(OCCF)cc(-c3cccc(F)c3)cc12] |
| PFP-00549 | 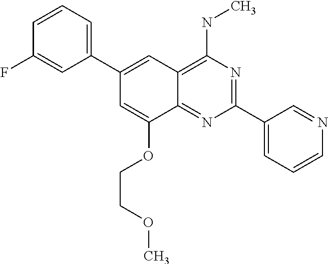 [SMILES: CNc1nc(-c2cccnc2)nc2c(OCCOC)cc(-c3cccc(F)c3)cc12] |
| PFP-00550 | 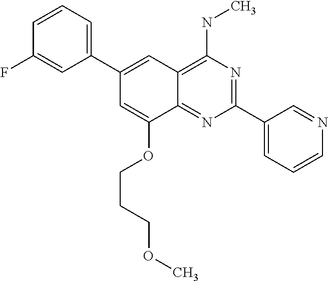 [SMILES: CNc1nc(-c2cccnc2)nc2c(OCCCOC)cc(-c3cccc(F)c3)cc12] |
| PFP-00551 | 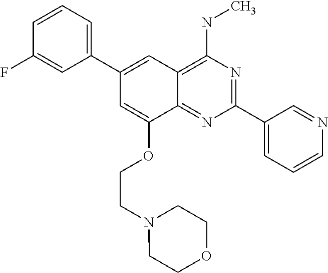 [SMILES: CNc1nc(-c2cccnc2)nc2c(OCCN3CCOCC3)cc(-c3cccc(F)c3)cc12] |
| PFP-00552 | 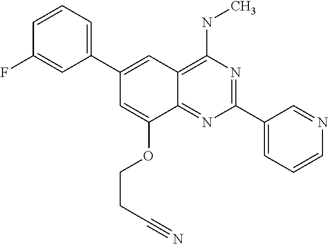 [SMILES: CNc1nc(-c2cccnc2)nc2c(OCCC#N)cc(-c3cccc(F)c3)cc12] |
| PFP-00553 | 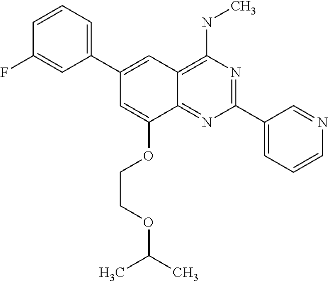 [SMILES: CNc1nc(-c2cccnc2)nc2c(OCCOC(C)C)cc(-c3cccc(F)c3)cc12] |
| PFP-00554 | 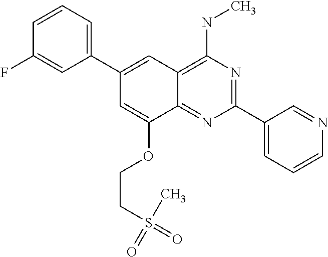 [SMILES: CNc1nc(-c2cccnc2)nc2c(OCCS(C)(=O)=O)cc(-c3cccc(F)c3)cc12] |
| PFP-00555 | 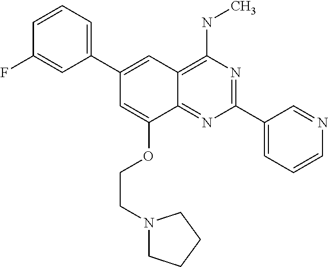 [SMILES: CNc1nc(-c2cccnc2)nc2c(OCCN3CCCC3)cc(-c3cccc(F)c3)cc12] |
| PFP-00556 | 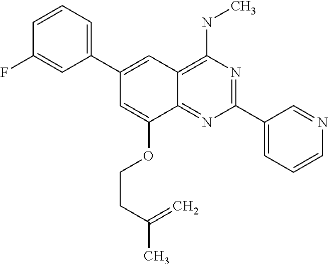 [SMILES: C=C(C)CCOc1cc(-c2cccc(F)c2)cc2c(NC)nc(-c3cccnc3)nc12] |
| PFP-00557 | 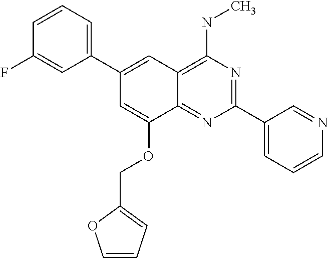 [SMILES: CNc1nc(-c2cccnc2)nc2c(OCc3ccco3)cc(-c3cccc(F)c3)cc12] |
| PFP-00558 | 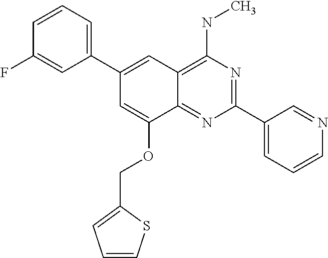 [SMILES: CNc1nc(-c2cccnc2)nc2c(OCc3cccs3)cc(-c3cccc(F)c3)cc12] |
| PFP-00559 | 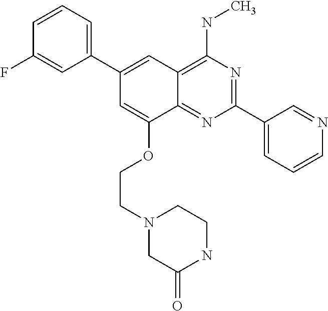 [SMILES: CNc1nc(-c2cccnc2)nc2c(OCCN3CCNC(=O)C3)cc(-c3cccc(F)c3)cc12] |
| PFP-00560 | 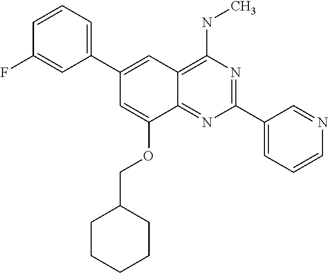 [SMILES: CNc1nc(-c2cccnc2)nc2c(OCC3CCCCC3)cc(-c3cccc(F)c3)cc12] |
| PFP-00561 | 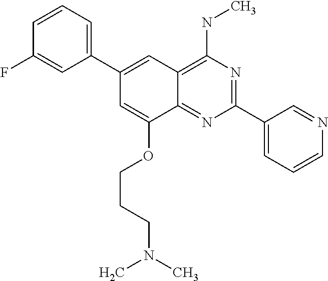 [SMILES: CNc1nc(-c2cccnc2)nc2c(OCCCN(C)C)cc(-c3cccc(F)c3)cc12] |
| PFP-00562 | 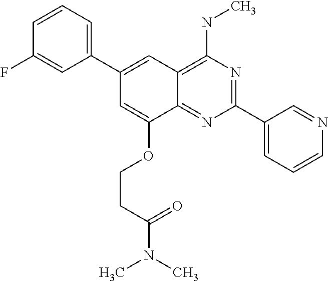 [SMILES: CNc1nc(-c2cccnc2)nc2c(OCCC(=O)N(C)C)cc(-c3cccc(F)c3)cc12] |
| PFP-00563 | 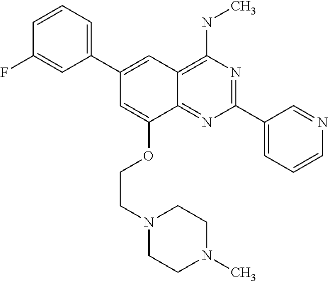 [SMILES: CNc1nc(-c2cccnc2)nc2c(OCCN3CCN(C)CC3)cc(-c3cccc(F)c3)cc12] |
| PFP-00564 | 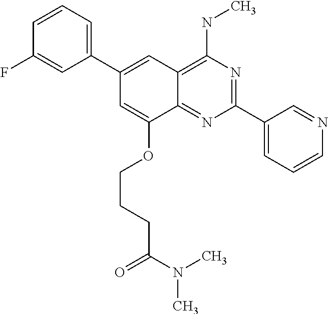 [SMILES: CNc1nc(-c2cccnc2)nc2c(OCCCC(=O)N(C)C)cc(-c3cccc(F)c3)cc12] |
| PFP-00565 | 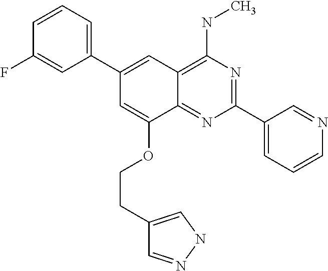 [SMILES: CNc1nc(-c2cccnc2)nc2c(OCCc3cn[nH]c3)cc(-c3cccc(F)c3)cc12] |
| PFP-00566 | 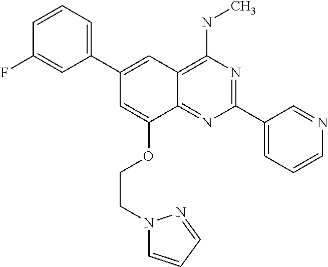 [SMILES: CNc1nc(-c2cccnc2)nc2c(OCCn3cccn3)cc(-c3cccc(F)c3)cc12] |
| PFP-00567 | 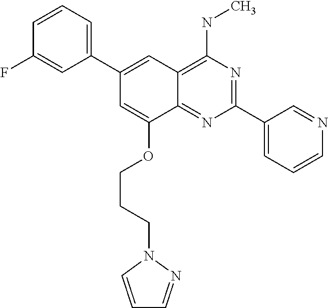 [SMILES: CNc1nc(-c2cccnc2)nc2c(OCCCn3cccn3)cc(-c3cccc(F)c3)cc12] |
| PFP-00568 | 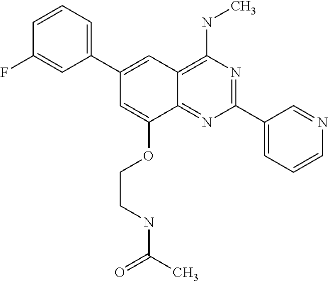 [SMILES: CNc1nc(-c2cccnc2)nc2c(OCCNC(C)=O)cc(-c3cccc(F)c3)cc12] |
| PFP-00569 | 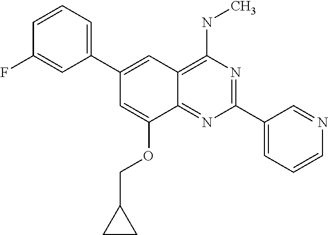 [SMILES: CNc1nc(-c2cccnc2)nc2c(OCC3CC3)cc(-c3cccc(F)c3)cc12] |
| PFP-00570 | 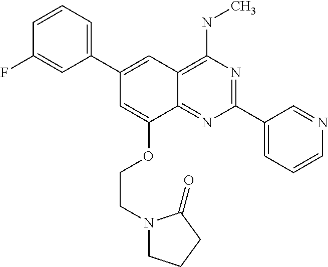 [SMILES: CNc1nc(-c2cccnc2)nc2c(OCCN3CCCC3=O)cc(-c3cccc(F)c3)cc12] |
| PFP-00571 | 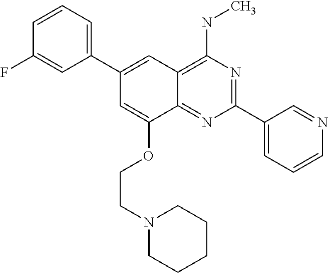 [SMILES: CNc1nc(-c2cccnc2)nc2c(OCCN3CCCCC3)cc(-c3cccc(F)c3)cc12] |
| PFP-00572 | 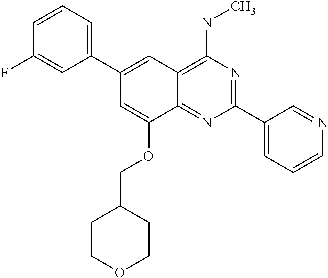 [SMILES: CNc1nc(-c2cccnc2)nc2c(OCC3CCOCC3)cc(-c3cccc(F)c3)cc12] |
| PFP-00575 | 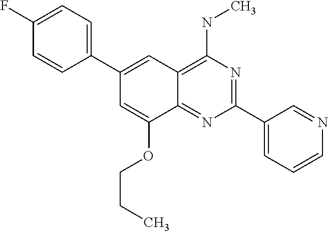 [SMILES: CCCOc1cc(-c2ccc(F)cc2)cc2c(NC)nc(-c3cccnc3)nc12] |
| PFP-00576 | 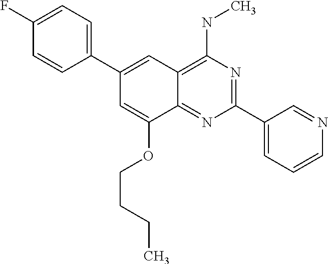 [SMILES: CCCCOc1cc(-c2ccc(F)cc2)cc2c(NC)nc(-c3cccnc3)nc12] |
| PFP-00577 | 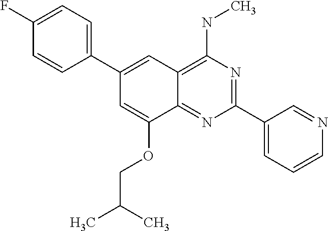 [SMILES: CNc1nc(-c2cccnc2)nc2c(OCC(C)C)cc(-c3ccc(F)cc3)cc12] |
| PFP-00578 | 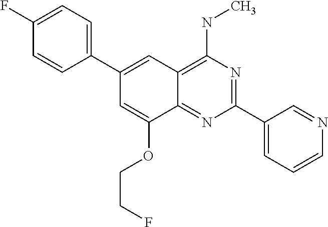 [SMILES: CNc1nc(-c2cccnc2)nc2c(OCCF)cc(-c3ccc(F)cc3)cc12] |
| PFP-00579 | 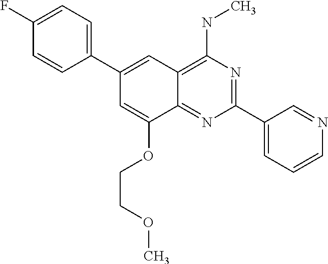 [SMILES: CNc1nc(-c2cccnc2)nc2c(OCCOC)cc(-c3ccc(F)cc3)cc12] |
| PFP-00580 | 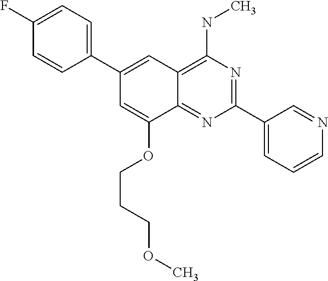 [SMILES: CNc1nc(-c2cccnc2)nc2c(OCCCOC)cc(-c3ccc(F)cc3)cc12] |
| PFP-00581 | 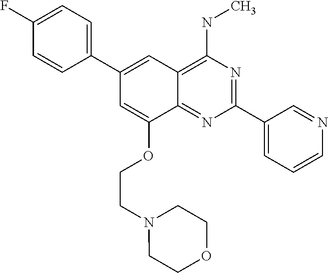 [SMILES: CNc1nc(-c2cccnc2)nc2c(OCCN3CCOCC3)cc(-c3ccc(F)cc3)cc12] |
| PFP-00582 | 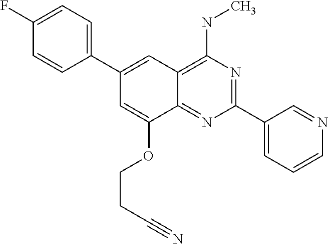 [SMILES: CNc1nc(-c2cccnc2)nc2c(OCCC#N)cc(-c3ccc(F)cc3)cc12] |
| PFP-00583 | 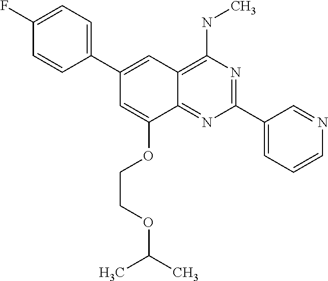 [SMILES: CNc1nc(-c2cccnc2)nc2c(OCCOC(C)C)cc(-c3ccc(F)cc3)cc12] |
| PFP-00584 | 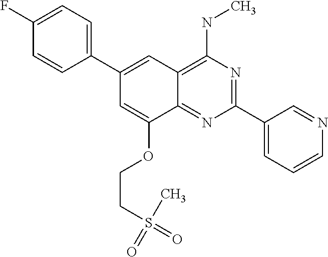 [SMILES: CNc1nc(-c2cccnc2)nc2c(OCCS(C)(=O)=O)cc(-c3ccc(F)cc3)cc12] |
| PFP-00585 | 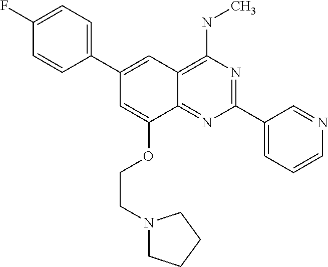 [SMILES: CNc1nc(-c2cccnc2)nc2c(OCCN3CCCC3)cc(-c3ccc(F)cc3)cc12] |
| PFP-00586 | 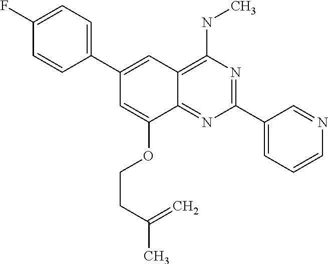 [SMILES: C=C(C)CCOc1cc(-c2ccc(F)cc2)cc2c(NC)nc(-c3cccnc3)nc12] |
| PFP-00587 | 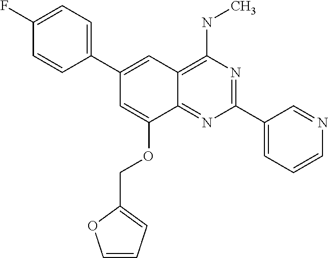 [SMILES: CNc1nc(-c2cccnc2)nc2c(OCc3ccco3)cc(-c3ccc(F)cc3)cc12] |
| PFP-00588 | 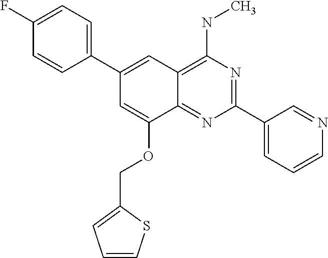 [SMILES: CNc1nc(-c2cccnc2)nc2c(OCc3cccs3)cc(-c3ccc(F)cc3)cc12] |
| PFP-00589 | 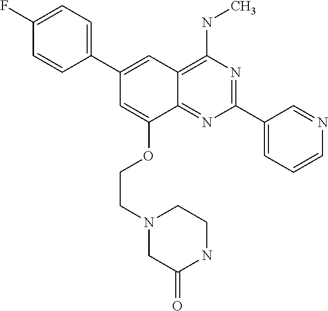 [SMILES: CNc1nc(-c2cccnc2)nc2c(OCCN3CCNC(=O)C3)cc(-c3ccc(F)cc3)cc12] |
| PFP-00590 | 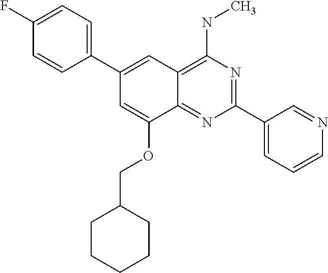 [SMILES: CNc1nc(-c2cccnc2)nc2c(OCC3CCCCC3)cc(-c3ccc(F)cc3)cc12] |
| PFP-00591 | 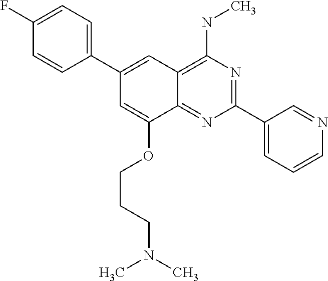 [SMILES: CNc1nc(-c2cccnc2)nc2c(OCCCN(C)C)cc(-c3ccc(F)cc3)cc12] |
| PFP-00592 | 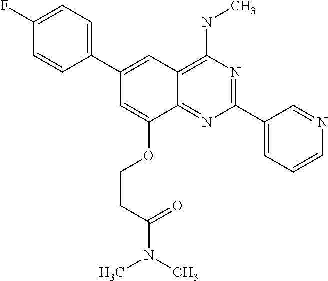 [SMILES: CNc1nc(-c2cccnc2)nc2c(OCCC(=O)N(C)C)cc(-c3ccc(F)cc3)cc12] |
| PFP-00593 | 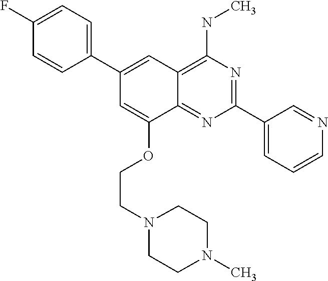 [SMILES: CNc1nc(-c2cccnc2)nc2c(OCCN3CCN(C)CC3)cc(-c3ccc(F)cc3)cc12] |
| PFP-00594 | 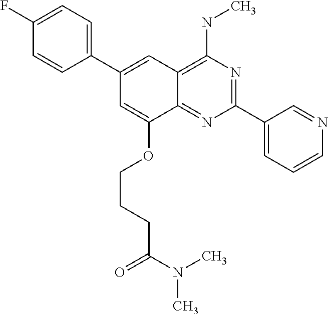 [SMILES: CNc1nc(-c2cccnc2)nc2c(OCCCC(=O)N(C)C)cc(-c3ccc(F)cc3)cc12] |
| PFP-00595 | 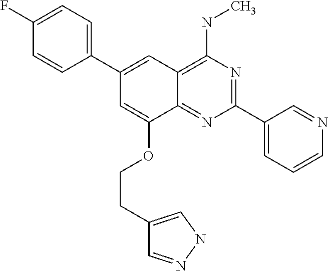 [SMILES: CNc1nc(-c2cccnc2)nc2c(OCCc3cn[nH]c3)cc(-c3ccc(F)cc3)cc12] |
| PFP-00596 | 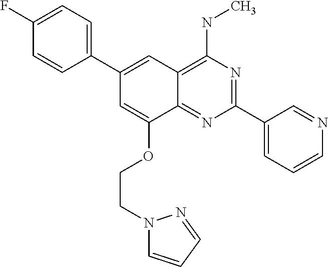 [SMILES: CNc1nc(-c2cccnc2)nc2c(OCCn3cccn3)cc(-c3ccc(F)cc3)cc12] |
| PFP-00597 | 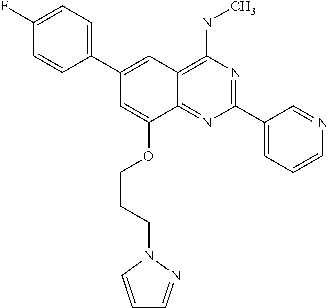 [SMILES: CNc1nc(-c2cccnc2)nc2c(OCCCn3cccn3)cc(-c3ccc(F)cc3)cc12] |
| PFP-00598 | 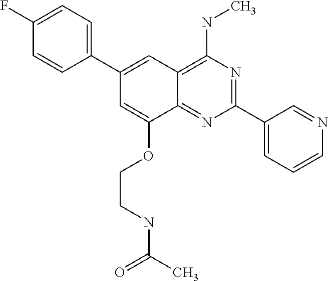 [SMILES: CNc1nc(-c2cccnc2)nc2c(OCCNC(C)=O)cc(-c3ccc(F)cc3)cc12] |
| PFP-00599 | 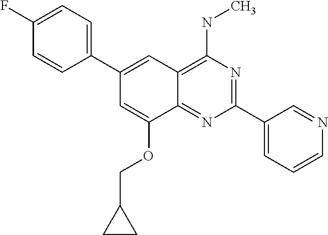 [SMILES: CNc1nc(-c2cccnc2)nc2c(OCC3CC3)cc(-c3ccc(F)cc3)cc12] |
| PFP-00600 | 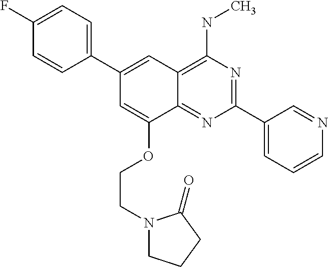 [SMILES: CNc1nc(-c2cccnc2)nc2c(OCCN3CCCC3=O)cc(-c3ccc(F)cc3)cc12] |
| PFP-00601 | 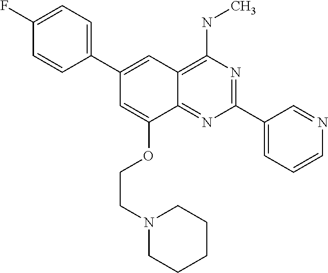 [SMILES: CNc1nc(-c2cccnc2)nc2c(OCCN3CCCCC3)cc(-c3ccc(F)cc3)cc12] |
| PFP-00602 | 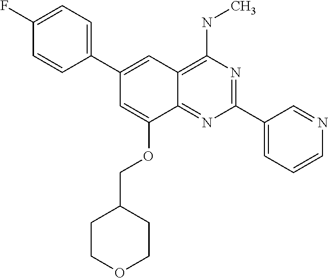 [SMILES: CNc1nc(-c2cccnc2)nc2c(OCC3CCOCC3)cc(-c3ccc(F)cc3)cc12] |
| PFP-00605 | 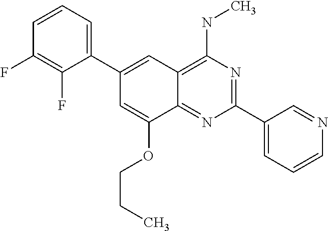 [SMILES: CCCOc1cc(-c2cccc(F)c2F)cc2c(NC)nc(-c3cccnc3)nc12] |
| PFP-00606 | 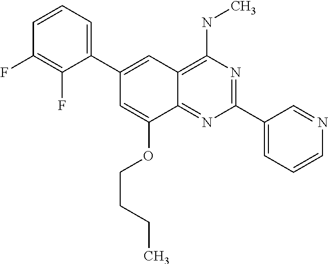 [SMILES: CCCCOc1cc(-c2cccc(F)c2F)cc2c(NC)nc(-c3cccnc3)nc12] |
| PFP-00607 | 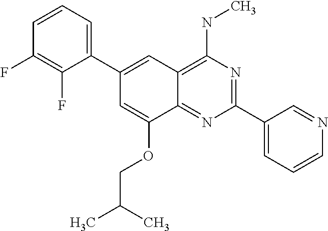 [SMILES: CNc1nc(-c2cccnc2)nc2c(OCC(C)C)cc(-c3cccc(F)c3F)cc12] |
| PFP-00608 | 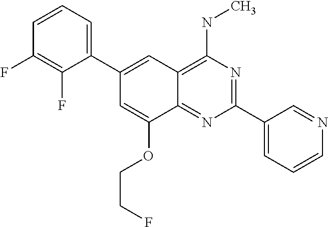 [SMILES: CNc1nc(-c2cccnc2)nc2c(OCCF)cc(-c3cccc(F)c3F)cc12] |
| PFP-00609 | 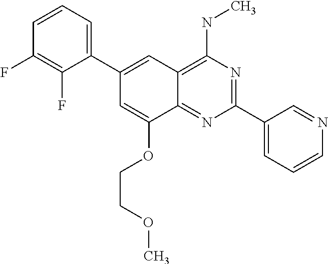 [SMILES: CNc1nc(-c2cccnc2)nc2c(OCCOC)cc(-c3cccc(F)c3F)cc12] |
| PFP-00610 | 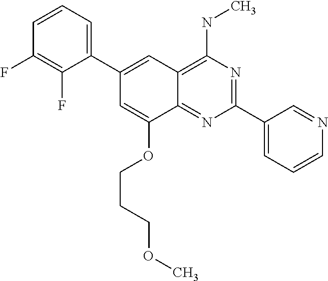 [SMILES: CNc1nc(-c2cccnc2)nc2c(OCCCOC)cc(-c3cccc(F)c3F)cc12] |
| PFP-00611 | 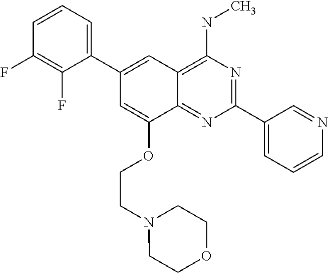 [SMILES: CNc1nc(-c2cccnc2)nc2c(OCCN3CCOCC3)cc(-c3cccc(F)c3F)cc12] |
| PFP-00612 | 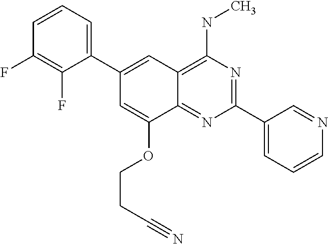 [SMILES: CNc1nc(-c2cccnc2)nc2c(OCCC#N)cc(-c3cccc(F)c3F)cc12] |
| PFP-00613 | 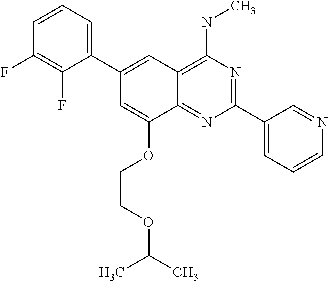 [SMILES: CNc1nc(-c2cccnc2)nc2c(OCCOC(C)C)cc(-c3cccc(F)c3F)cc12] |
| PFP-00614 | 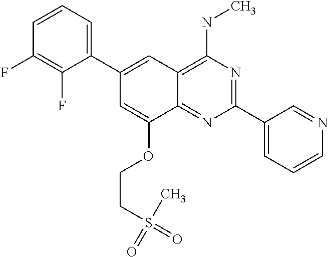 [SMILES: CNc1nc(-c2cccnc2)nc2c(OCCS(C)(=O)=O)cc(-c3cccc(F)c3F)cc12] |
| PFP-00615 | 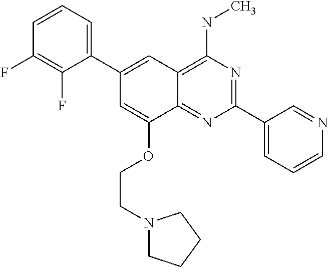 [SMILES: CNc1nc(-c2cccnc2)nc2c(OCCN3CCCC3)cc(-c3cccc(F)c3F)cc12] |
| PFP-00616 | 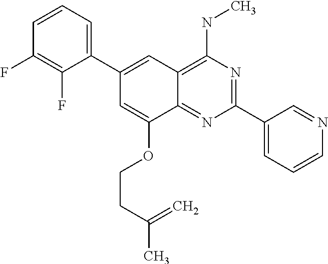 [SMILES: C=C(C)CCOc1cc(-c2cccc(F)c2F)cc2c(NC)nc(-c3cccnc3)nc12] |
| PFP-00617 | 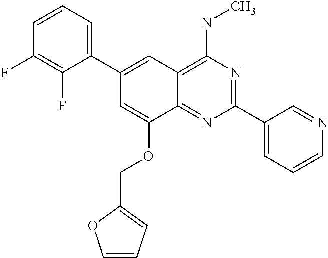 [SMILES: CNc1nc(-c2cccnc2)nc2c(OCc3ccco3)cc(-c3cccc(F)c3F)cc12] |
| PFP-00618 | 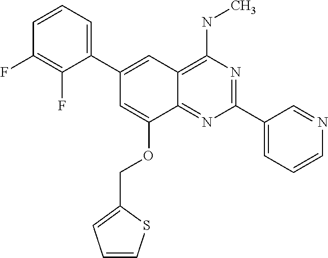 [SMILES: CNc1nc(-c2cccnc2)nc2c(OCc3cccs3)cc(-c3cccc(F)c3F)cc12] |
| PFP-00619 | 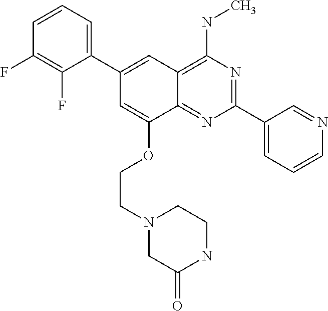 [SMILES: CNc1nc(-c2cccnc2)nc2c(OCCN3CCNC(=O)C3)cc(-c3cccc(F)c3F)cc12] |
| PFP-00620 | 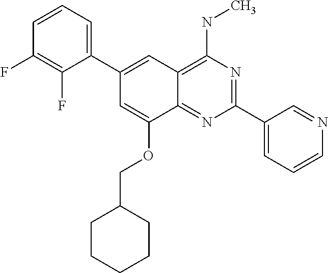 [SMILES: CNc1nc(-c2cccnc2)nc2c(OCC3CCCCC3)cc(-c3cccc(F)c3F)cc12] |
| PFP-00621 | 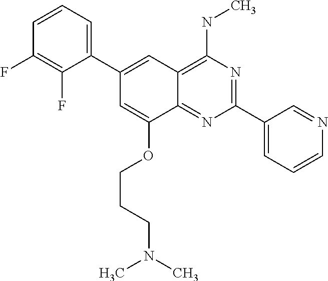 [SMILES: CNc1nc(-c2cccnc2)nc2c(OCCCN(C)C)cc(-c3cccc(F)c3F)cc12] |
| PFP-00622 | 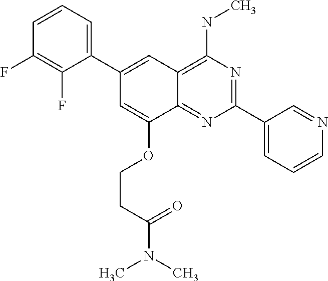 [SMILES: CNc1nc(-c2cccnc2)nc2c(OCCC(=O)N(C)C)cc(-c3cccc(F)c3F)cc12] |
| PFP-00623 | 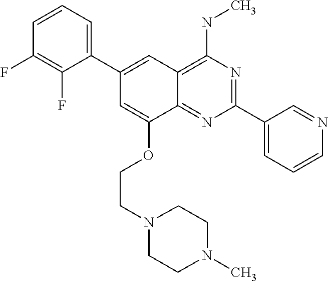 [SMILES: CNc1nc(-c2cccnc2)nc2c(OCCN3CCN(C)CC3)cc(-c3cccc(F)c3F)cc12] |
| PFP-00624 | 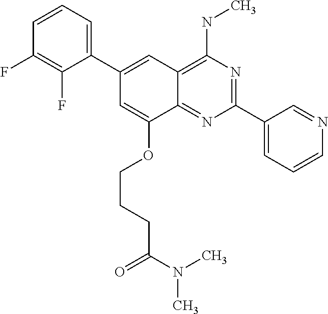 [SMILES: CNc1nc(-c2cccnc2)nc2c(OCCCC(=O)N(C)C)cc(-c3cccc(F)c3F)cc12] |
| PFP-00625 | 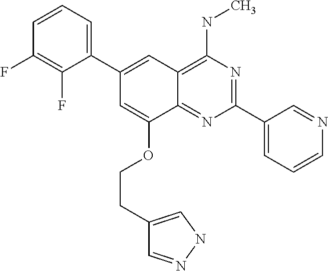 [SMILES: CNc1nc(-c2cccnc2)nc2c(OCCc3cn[nH]c3)cc(-c3cccc(F)c3F)cc12] |
| PFP-00626 | 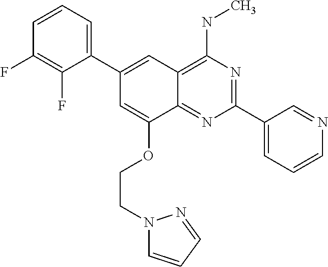 [SMILES: CNc1nc(-c2cccnc2)nc2c(OCCn3cccn3)cc(-c3cccc(F)c3F)cc12] |
| PFP-00627 | 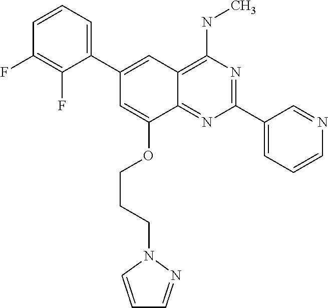 [SMILES: CNc1nc(-c2cccnc2)nc2c(OCCCn3cccn3)cc(-c3cccc(F)c3F)cc12] |
| PFP-00628 | 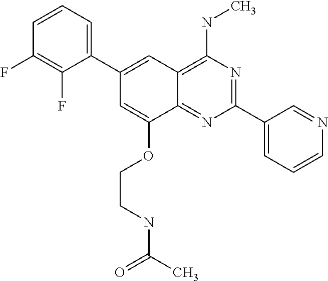 [SMILES: CNc1nc(-c2cccnc2)nc2c(OCCNC(C)=O)cc(-c3cccc(F)c3F)cc12] |
| PFP-00629 | 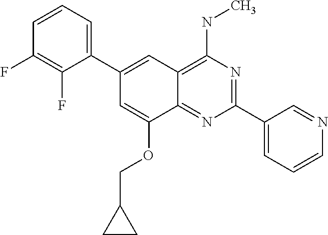 [SMILES: CNc1nc(-c2cccnc2)nc2c(OCC3CC3)cc(-c3cccc(F)c3F)cc12] |
| PFP-00630 | 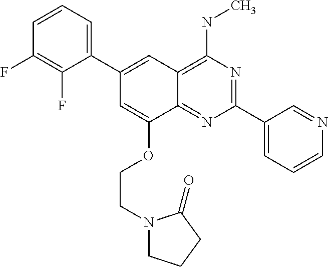 [SMILES: CNc1nc(-c2cccnc2)nc2c(OCCN3CCCC3=O)cc(-c3cccc(F)c3F)cc12] |
| PFP-00631 | 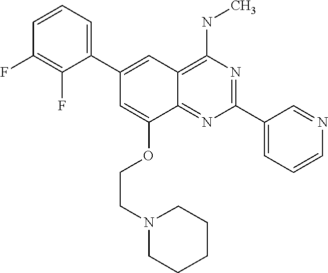 [SMILES: CNc1nc(-c2cccnc2)nc2c(OCCN3CCCCC3)cc(-c3cccc(F)c3F)cc12] |
| PFP-00632 | 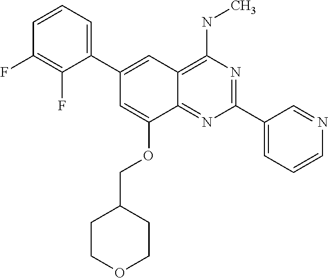 [SMILES: CNc1nc(-c2cccnc2)nc2c(OCC3CCOCC3)cc(-c3cccc(F)c3F)cc12] |
| PFP-00635 | 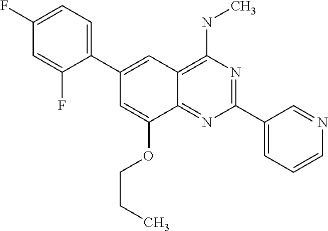 [SMILES: CCCOc1cc(-c2ccc(F)cc2F)cc2c(NC)nc(-c3cccnc3)nc12] |
| PFP-00636 | 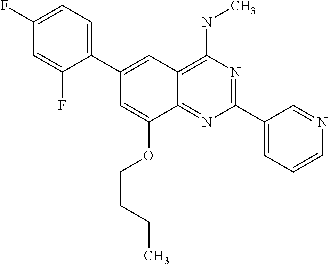 [SMILES: CCCCOc1cc(-c2ccc(F)cc2F)cc2c(NC)nc(-c3cccnc3)nc12] |
| PFP-00637 | 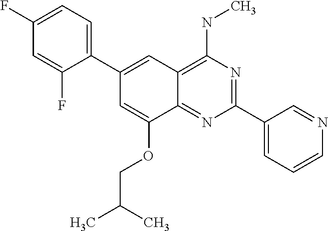 [SMILES: CNc1nc(-c2cccnc2)nc2c(OCC(C)C)cc(-c3ccc(F)cc3F)cc12] |
| PFP-00638 | 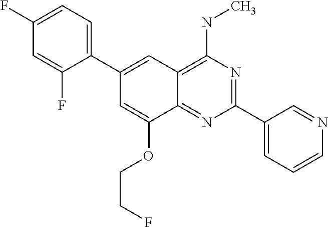 [SMILES: CNc1nc(-c2cccnc2)nc2c(OCCF)cc(-c3ccc(F)cc3F)cc12] |
| PFP-00639 | 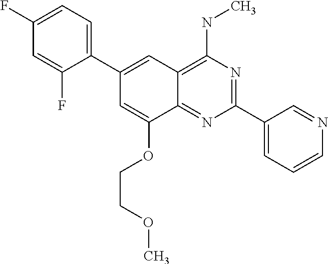 [SMILES: CNc1nc(-c2cccnc2)nc2c(OCCOC)cc(-c3ccc(F)cc3F)cc12] |
| PFP-00640 | 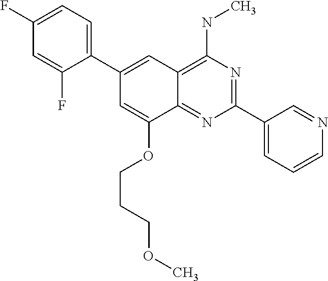 [SMILES: CNc1nc(-c2cccnc2)nc2c(OCCCOC)cc(-c3ccc(F)cc3F)cc12] |
| PFP-00641 | 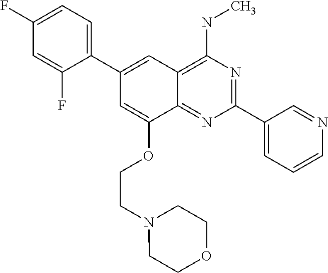 [SMILES: CNc1nc(-c2cccnc2)nc2c(OCCN3CCOCC3)cc(-c3ccc(F)cc3F)cc12] |
| PFP-00642 | 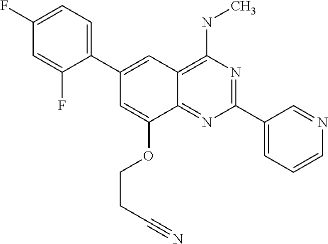 [SMILES: CNc1nc(-c2cccnc2)nc2c(OCCC#N)cc(-c3ccc(F)cc3F)cc12] |
| PFP-00643 | 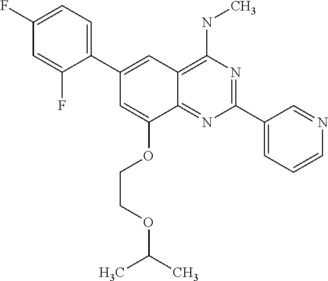 [SMILES: CNc1nc(-c2cccnc2)nc2c(OCCOC(C)C)cc(-c3ccc(F)cc3F)cc12] |
| PFP-00644 | 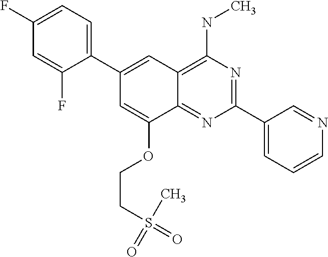 [SMILES: CNc1nc(-c2cccnc2)nc2c(OCCS(C)(=O)=O)cc(-c3ccc(F)cc3F)cc12] |
| PFP-00645 | 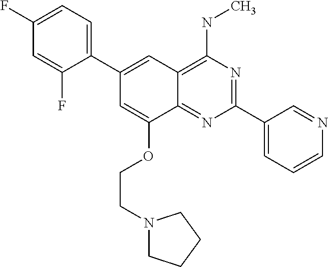 [SMILES: CNc1nc(-c2cccnc2)nc2c(OCCN3CCCC3)cc(-c3ccc(F)cc3F)cc12] |
| PFP-00646 | 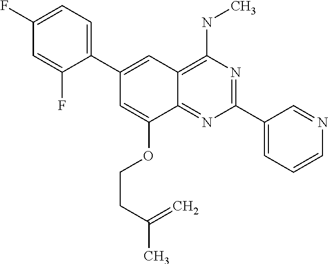 [SMILES: C=C(C)CCOc1cc(-c2ccc(F)cc2F)cc2c(NC)nc(-c3cccnc3)nc12] |
| PFP-00647 | 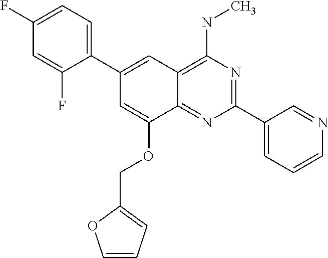 [SMILES: CNc1nc(-c2cccnc2)nc2c(OCc3ccco3)cc(-c3ccc(F)cc3F)cc12] |
| PFP-00648 | 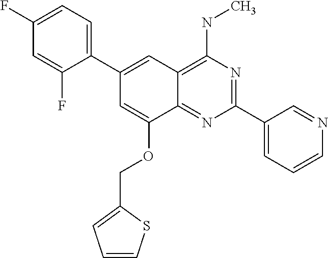 [SMILES: CNc1nc(-c2cccnc2)nc2c(OCc3cccs3)cc(-c3ccc(F)cc3F)cc12] |
| PFP-00649 | 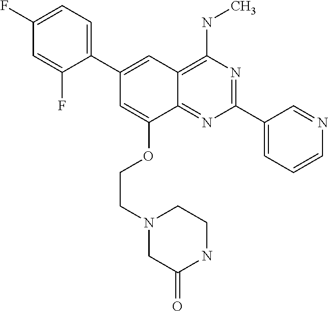 [SMILES: CNc1nc(-c2cccnc2)nc2c(OCCN3CCNC(=O)C3)cc(-c3ccc(F)cc3F)cc12] |
| PFP-00650 | 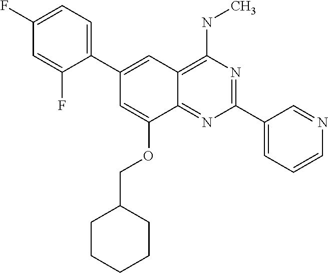 [SMILES: CNc1nc(-c2cccnc2)nc2c(OCC3CCCCC3)cc(-c3ccc(F)cc3F)cc12] |
| PFP-00651 | 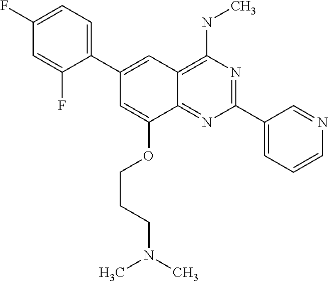 [SMILES: CNc1nc(-c2cccnc2)nc2c(OCCCN(C)C)cc(-c3ccc(F)cc3F)cc12] |
| PFP-00652 | 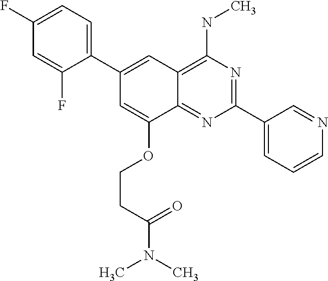 [SMILES: CNc1nc(-c2cccnc2)nc2c(OCCC(=O)N(C)C)cc(-c3ccc(F)cc3F)cc12] |
| PFP-00653 | 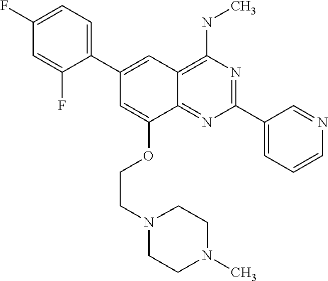 [SMILES: CNc1nc(-c2cccnc2)nc2c(OCCN3CCN(C)CC3)cc(-c3ccc(F)cc3F)cc12] |
| PFP-00654 | 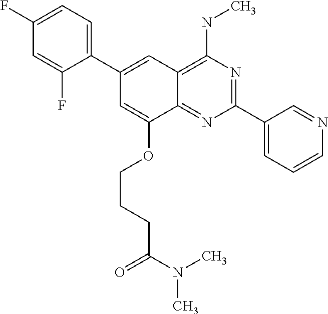 [SMILES: CNc1nc(-c2cccnc2)nc2c(OCCCC(=O)N(C)C)cc(-c3ccc(F)cc3F)cc12] |
| PFP-00655 | 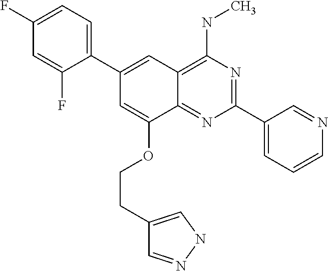 [SMILES: CNc1nc(-c2cccnc2)nc2c(OCCc3cn[nH]c3)cc(-c3ccc(F)cc3F)cc12] |
| PFP-00656 | 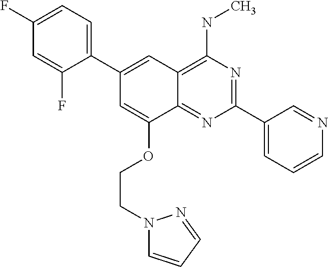 [SMILES: CNc1nc(-c2cccnc2)nc2c(OCCn3cccn3)cc(-c3ccc(F)cc3F)cc12] |
| PFP-00657 | 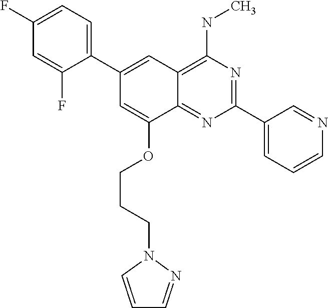 [SMILES: CNc1nc(-c2cccnc2)nc2c(OCCCn3cccn3)cc(-c3ccc(F)cc3F)cc12] |
| PFP-00658 | 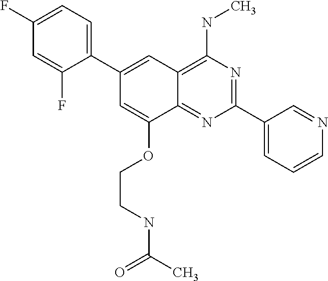 [SMILES: CNc1nc(-c2cccnc2)nc2c(OCCNC(C)=O)cc(-c3ccc(F)cc3F)cc12] |
| PFP-00659 | 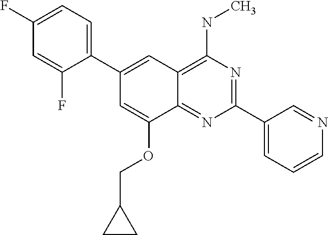 [SMILES: CNc1nc(-c2cccnc2)nc2c(OCC3CC3)cc(-c3ccc(F)cc3F)cc12] |
| PFP-00660 | 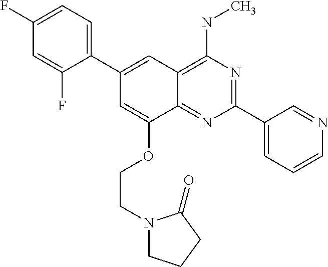 [SMILES: CNc1nc(-c2cccnc2)nc2c(OCCN3CCCC3=O)cc(-c3ccc(F)cc3F)cc12] |
| PFP-00661 | 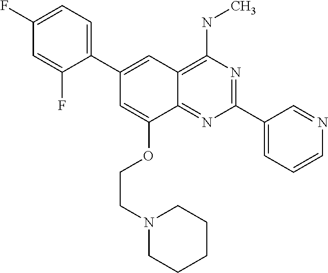 [SMILES: CNc1nc(-c2cccnc2)nc2c(OCCN3CCCCC3)cc(-c3ccc(F)cc3F)cc12] |
| PFP-00662 | 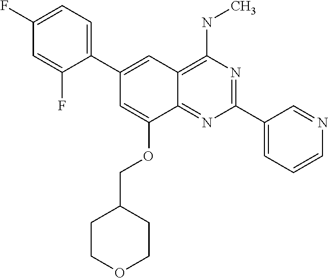 [SMILES: CNc1nc(-c2cccnc2)nc2c(OCC3CCOCC3)cc(-c3ccc(F)cc3F)cc12] |
| PFP-00665 | 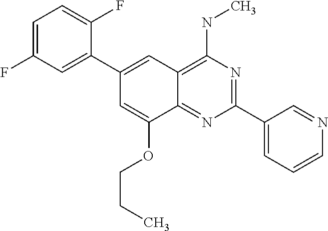 [SMILES: CCCOc1cc(-c2cc(F)ccc2F)cc2c(NC)nc(-c3cccnc3)nc12] |
| PFP-00666 | 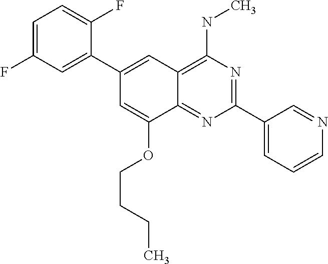 [SMILES: CCCCOc1cc(-c2cc(F)ccc2F)cc2c(NC)nc(-c3cccnc3)nc12] |
| PFP-00667 | 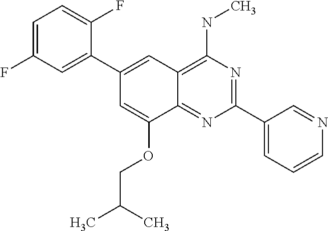 [SMILES: CNc1nc(-c2cccnc2)nc2c(OCC(C)C)cc(-c3cc(F)ccc3F)cc12] |
| PFP-00668 | 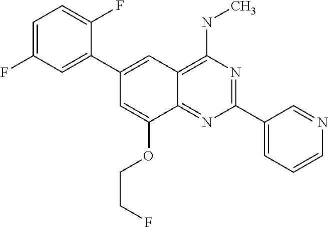 [SMILES: CNc1nc(-c2cccnc2)nc2c(OCCF)cc(-c3cc(F)ccc3F)cc12] |
| PFP-00669 | 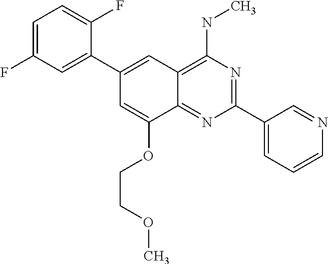 [SMILES: CNc1nc(-c2cccnc2)nc2c(OCCOC)cc(-c3cc(F)ccc3F)cc12] |
| PFP-00670 | 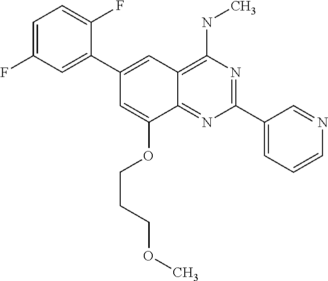 [SMILES: CNc1nc(-c2cccnc2)nc2c(OCCCOC)cc(-c3cc(F)ccc3F)cc12] |
| PFP-00671 | 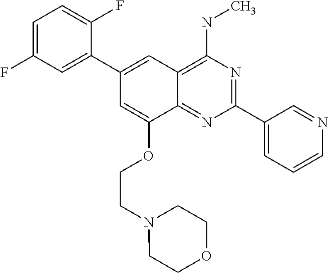 [SMILES: CNc1nc(-c2cccnc2)nc2c(OCCN3CCOCC3)cc(-c3cc(F)ccc3F)cc12] |
| PFP-00672 | 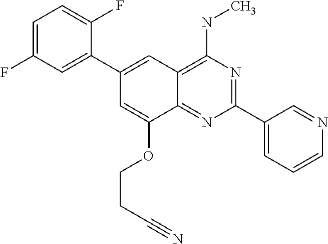 [SMILES: CNc1nc(-c2cccnc2)nc2c(OCCC#N)cc(-c3cc(F)ccc3F)cc12] |
| PFP-00673 | 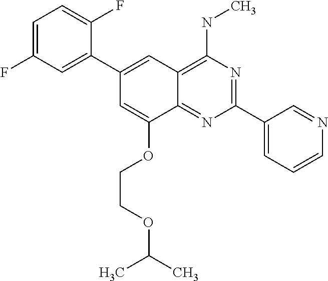 [SMILES: CNc1nc(-c2cccnc2)nc2c(OCCOC(C)C)cc(-c3cc(F)ccc3F)cc12] |
| PFP-00674 | 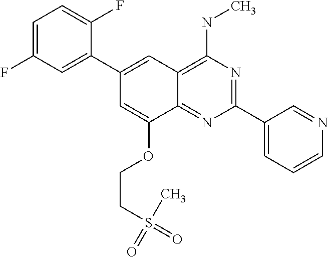 [SMILES: CNc1nc(-c2cccnc2)nc2c(OCCS(C)(=O)=O)cc(-c3cc(F)ccc3F)cc12] |
| PFP-00675 | 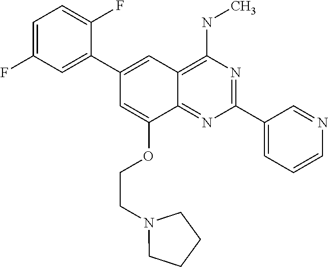 [SMILES: CNc1nc(-c2cccnc2)nc2c(OCCN3CCCC3)cc(-c3cc(F)ccc3F)cc12] |
| PFP-00676 | 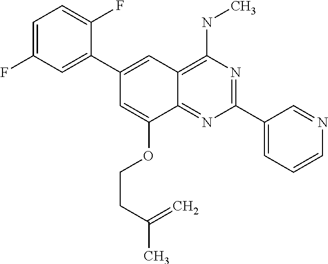 [SMILES: C=C(C)CCOc1cc(-c2cc(F)ccc2F)cc2c(NC)nc(-c3cccnc3)nc12] |
| PFP-00677 | 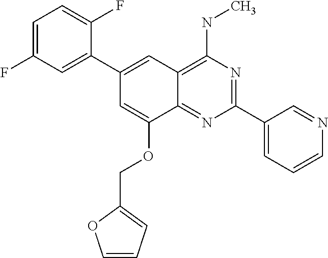 [SMILES: CNc1nc(-c2cccnc2)nc2c(OCc3ccco3)cc(-c3cc(F)ccc3F)cc12] |
| PFP-00678 | 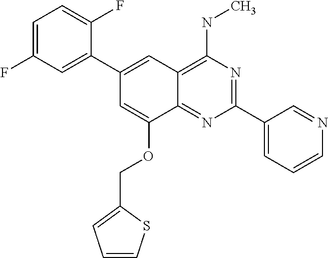 [SMILES: CNc1nc(-c2cccnc2)nc2c(OCc3cccs3)cc(-c3cc(F)ccc3F)cc12] |
| PFP-00679 | 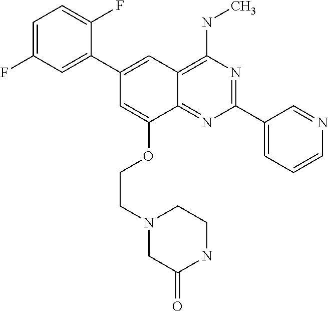 [SMILES: CNc1nc(-c2cccnc2)nc2c(OCCN3CCNC(=O)C3)cc(-c3cc(F)ccc3F)cc12] |
| PFP-00680 | 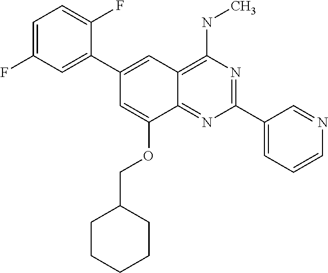 [SMILES: CNc1nc(-c2cccnc2)nc2c(OCC3CCCCC3)cc(-c3cc(F)ccc3F)cc12] |
| PFP-00681 | 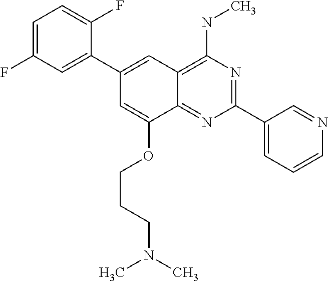 [SMILES: CNc1nc(-c2cccnc2)nc2c(OCCCN(C)C)cc(-c3cc(F)ccc3F)cc12] |
| PFP-00682 | 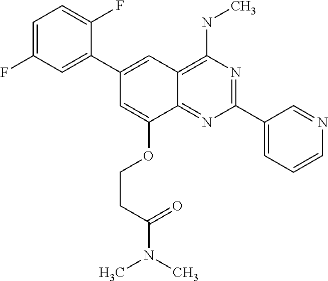 [SMILES: CNc1nc(-c2cccnc2)nc2c(OCCC(=O)N(C)C)cc(-c3cc(F)ccc3F)cc12] |
| PFP-00683 | 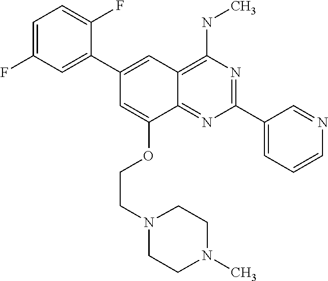 [SMILES: CNc1nc(-c2cccnc2)nc2c(OCCN3CCN(C)CC3)cc(-c3cc(F)ccc3F)cc12] |
| PFP-00684 | 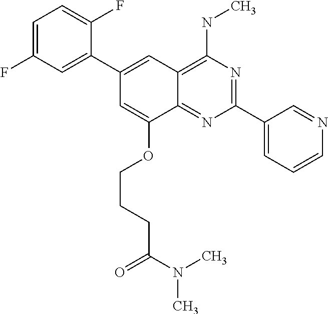 [SMILES: CNc1nc(-c2cccnc2)nc2c(OCCCC(=O)N(C)C)cc(-c3cc(F)ccc3F)cc12] |
| PFP-00685 | 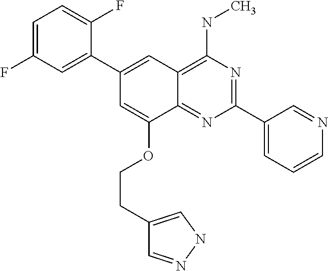 [SMILES: CNc1nc(-c2cccnc2)nc2c(OCCc3cn[nH]c3)cc(-c3cc(F)ccc3F)cc12] |
| PFP-00686 | 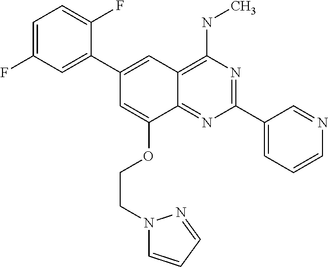 [SMILES: CNc1nc(-c2cccnc2)nc2c(OCCn3cccn3)cc(-c3cc(F)ccc3F)cc12] |
| PFP-00687 | 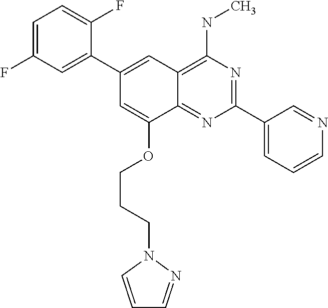 [SMILES: CNc1nc(-c2cccnc2)nc2c(OCCCn3cccn3)cc(-c3cc(F)ccc3F)cc12] |
| PFP-00688 | 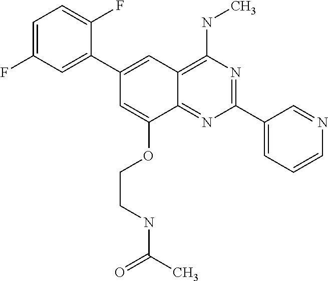 [SMILES: CNc1nc(-c2cccnc2)nc2c(OCCNC(C)=O)cc(-c3cc(F)ccc3F)cc12] |
| PFP-00689 | 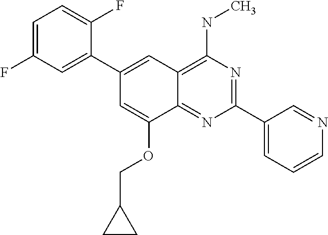 [SMILES: CNc1nc(-c2cccnc2)nc2c(OCC3CC3)cc(-c3cc(F)ccc3F)cc12] |
| PFP-00690 | 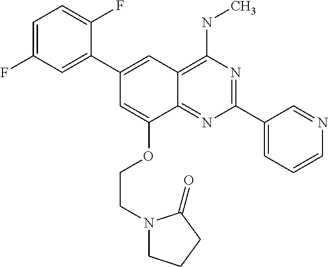 [SMILES: CNc1nc(-c2cccnc2)nc2c(OCCN3CCCC3=O)cc(-c3cc(F)ccc3F)cc12] |
| PFP-00691 | 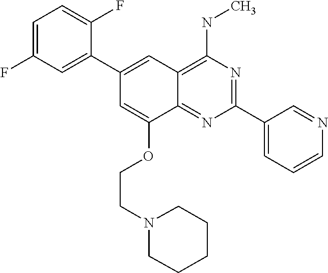 [SMILES: CNc1nc(-c2cccnc2)nc2c(OCCN3CCCCC3)cc(-c3cc(F)ccc3F)cc12] |
| PFP-00692 | 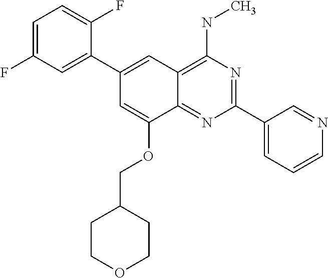 [SMILES: CNc1nc(-c2cccnc2)nc2c(OCC3CCOCC3)cc(-c3cc(F)ccc3F)cc12] |
| PFP-00695 | 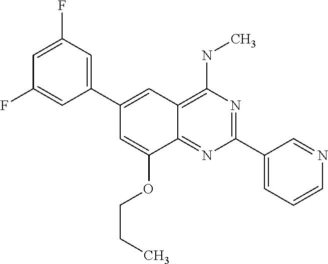 [SMILES: CCCOc1cc(-c2cc(F)cc(F)c2)cc2c(NC)nc(-c3cccnc3)nc12] |
| PFP-00696 | 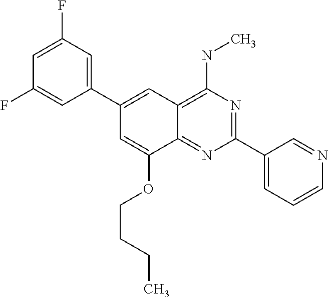 [SMILES: CCCCOc1cc(-c2cc(F)cc(F)c2)cc2c(NC)nc(-c3cccnc3)nc12] |
| PFP-00697 | 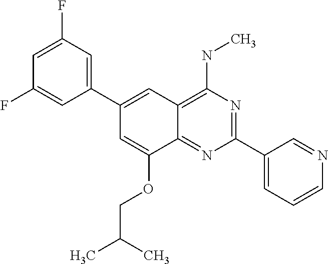 [SMILES: CNc1nc(-c2cccnc2)nc2c(OCC(C)C)cc(-c3cc(F)cc(F)c3)cc12] |
| PFP-00698 | 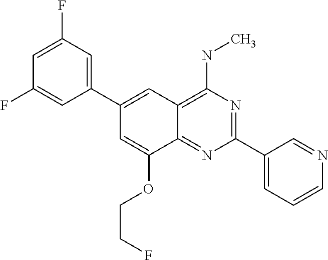 [SMILES: CNc1nc(-c2cccnc2)nc2c(OCCF)cc(-c3cc(F)cc(F)c3)cc12] |
| PFP-00699 | 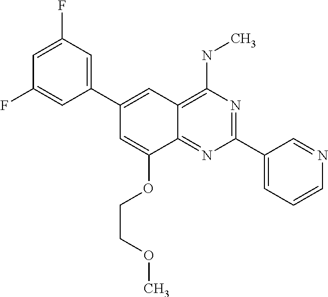 [SMILES: CNc1nc(-c2cccnc2)nc2c(OCCOC)cc(-c3cc(F)cc(F)c3)cc12] |
| PFP-00700 | 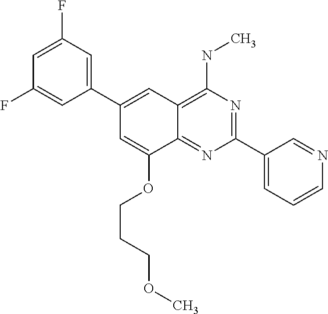 [SMILES: CNc1nc(-c2cccnc2)nc2c(OCCCOC)cc(-c3cc(F)cc(F)c3)cc12] |
| PFP-00701 | 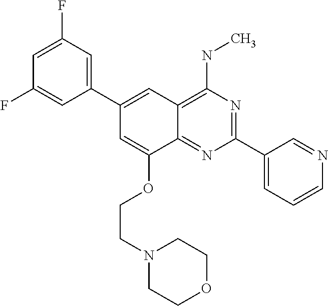 [SMILES: CNc1nc(-c2cccnc2)nc2c(OCCN3CCOCC3)cc(-c3cc(F)cc(F)c3)cc12] |
| PFP-00702 | 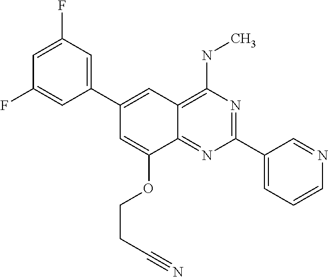 [SMILES: CNc1nc(-c2cccnc2)nc2c(OCCC#N)cc(-c3cc(F)cc(F)c3)cc12] |
| PFP-00703 | 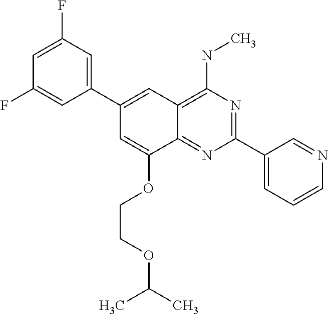 [SMILES: CNc1nc(-c2cccnc2)nc2c(OCCOC(C)C)cc(-c3cc(F)cc(F)c3)cc12] |
| PFP-00704 | 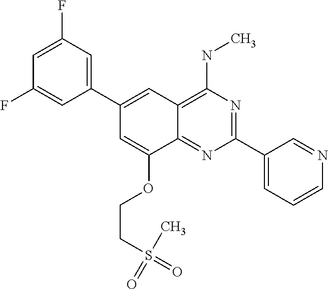 [SMILES: CNc1nc(-c2cccnc2)nc2c(OCCS(C)(=O)=O)cc(-c3cc(F)cc(F)c3)cc12] |
| PFP-00705 | 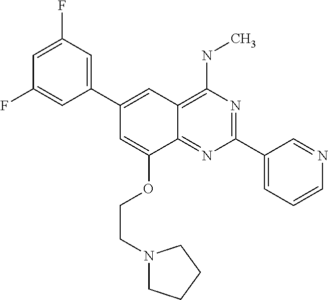 [SMILES: CNc1nc(-c2cccnc2)nc2c(OCCN3CCCC3)cc(-c3cc(F)cc(F)c3)cc12] |
| PFP-00706 | 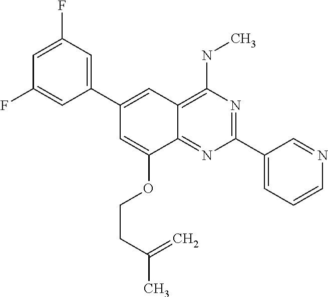 [SMILES: C=C(C)CCOc1cc(-c2cc(F)cc(F)c2)cc2c(NC)nc(-c3cccnc3)nc12] |
| PFP-00707 | 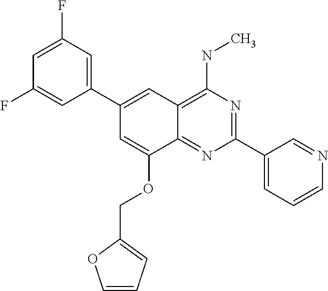 [SMILES: CNc1nc(-c2cccnc2)nc2c(OCc3ccco3)cc(-c3cc(F)cc(F)c3)cc12] |
| PFP-00708 | 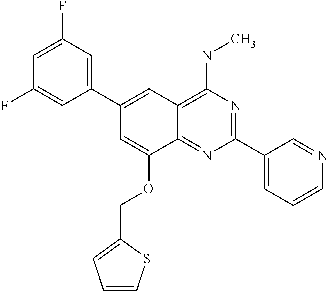 [SMILES: CNc1nc(-c2cccnc2)nc2c(OCc3cccs3)cc(-c3cc(F)cc(F)c3)cc12] |
| PFP-00709 | 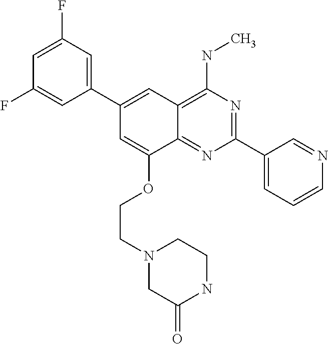 [SMILES: CNc1nc(-c2cccnc2)nc2c(OCCN3CCNC(=O)C3)cc(-c3cc(F)cc(F)c3)cc12] |
| PFP-00710 | 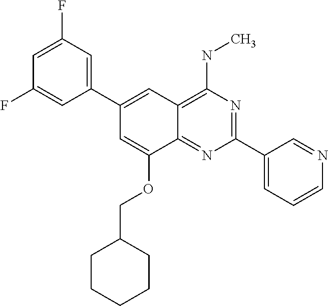 [SMILES: CNc1nc(-c2cccnc2)nc2c(OCC3CCCCC3)cc(-c3cc(F)cc(F)c3)cc12] |
| PFP-00711 | 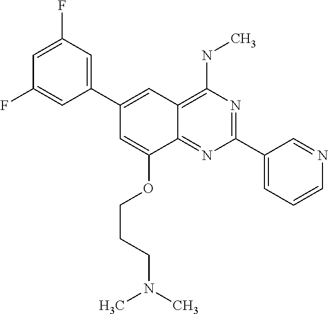 [SMILES: CNc1nc(-c2cccnc2)nc2c(OCCCN(C)C)cc(-c3cc(F)cc(F)c3)cc12] |
| PFP-00712 | 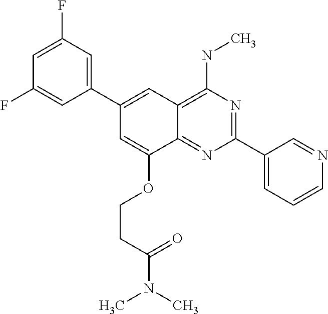 [SMILES: CNc1nc(-c2cccnc2)nc2c(OCCC(=O)N(C)C)cc(-c3cc(F)cc(F)c3)cc12] |
| PFP-00713 | 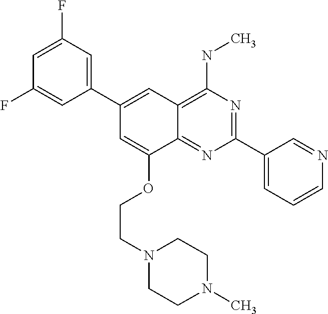 [SMILES: CNc1nc(-c2cccnc2)nc2c(OCCN3CCN(C)CC3)cc(-c3cc(F)cc(F)c3)cc12] |
| PFP-00714 | 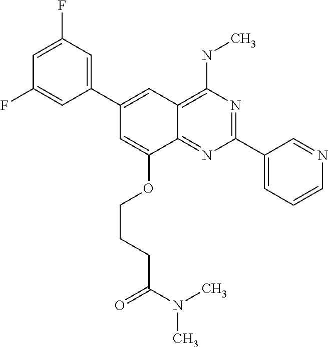 [SMILES: CNc1nc(-c2cccnc2)nc2c(OCCCC(=O)N(C)C)cc(-c3cc(F)cc(F)c3)cc12] |
| PFP-00715 | 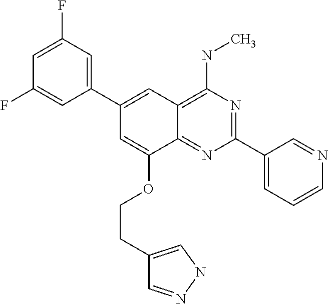 [SMILES: CNc1nc(-c2cccnc2)nc2c(OCCc3cn[nH]c3)cc(-c3cc(F)cc(F)c3)cc12] |
| PFP-00716 | 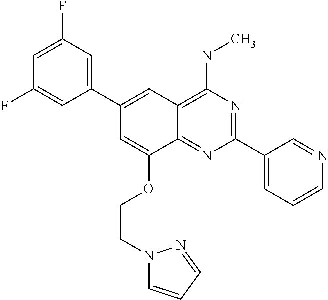 [SMILES: CNc1nc(-c2cccnc2)nc2c(OCCn3cccn3)cc(-c3cc(F)cc(F)c3)cc12] |
| PFP-00717 | 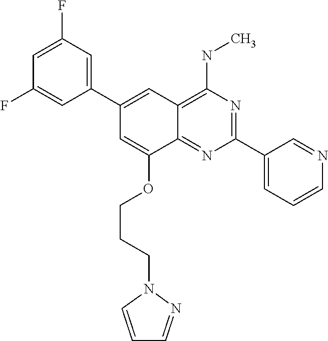 [SMILES: CNc1nc(-c2cccnc2)nc2c(OCCCn3cccn3)cc(-c3cc(F)cc(F)c3)cc12] |
| PFP-00718 | 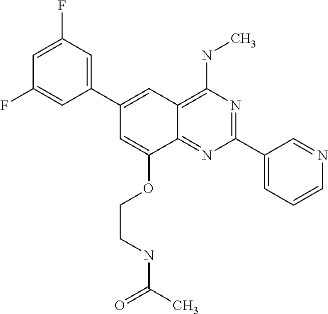 [SMILES: CNc1nc(-c2cccnc2)nc2c(OCCNC(C)=O)cc(-c3cc(F)cc(F)c3)cc12] |
| PFP-00719 | 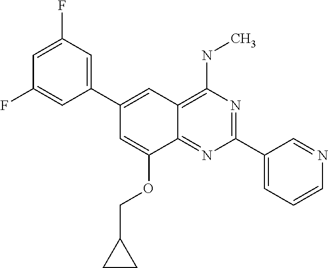 [SMILES: CNc1nc(-c2cccnc2)nc2c(OCC3CC3)cc(-c3cc(F)cc(F)c3)cc12] |
| PFP-00720 | 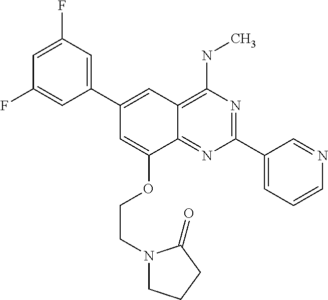 [SMILES: CNc1nc(-c2cccnc2)nc2c(OCCN3CCCC3=O)cc(-c3cc(F)cc(F)c3)cc12] |
| PFP-00721 | 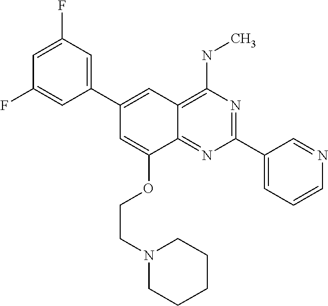 [SMILES: CNc1nc(-c2cccnc2)nc2c(OCCN3CCCCC3)cc(-c3cc(F)cc(F)c3)cc12] |
| PFP-00722 | 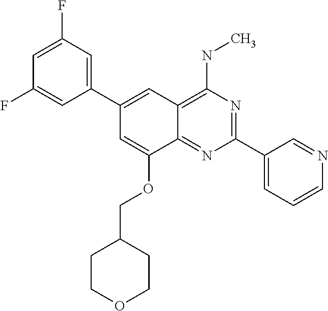 [SMILES: CNc1nc(-c2cccnc2)nc2c(OCC3CCOCC3)cc(-c3cc(F)cc(F)c3)cc12] |
| PFP-00725 | 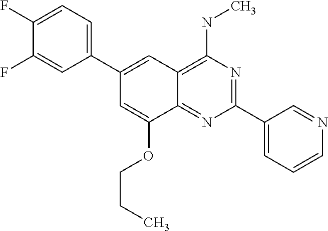 [SMILES: CCCOc1cc(-c2ccc(F)c(F)c2)cc2c(NC)nc(-c3cccnc3)nc12] |
| PFP-00726 | 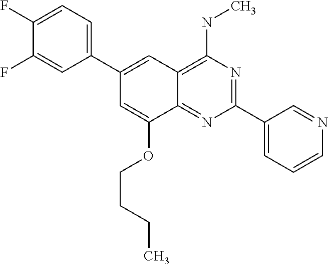 [SMILES: CCCCOc1cc(-c2ccc(F)c(F)c2)cc2c(NC)nc(-c3cccnc3)nc12] |
| PFP-00727 | 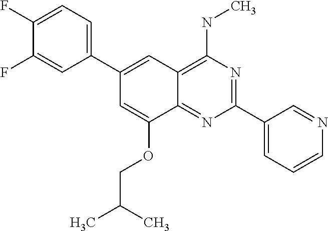 [SMILES: CNc1nc(-c2cccnc2)nc2c(OCC(C)C)cc(-c3ccc(F)c(F)c3)cc12] |
| PFP-00728 | 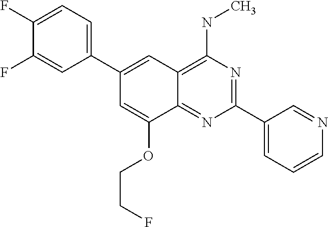 [SMILES: CNc1nc(-c2cccnc2)nc2c(OCCF)cc(-c3ccc(F)c(F)c3)cc12] |
| PFP-00729 | 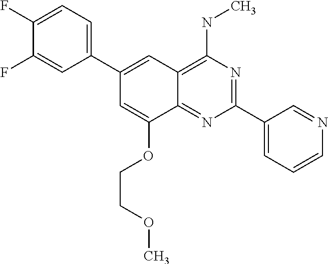 [SMILES: CNc1nc(-c2cccnc2)nc2c(OCCOC)cc(-c3ccc(F)c(F)c3)cc12] |
| PFP-00730 | 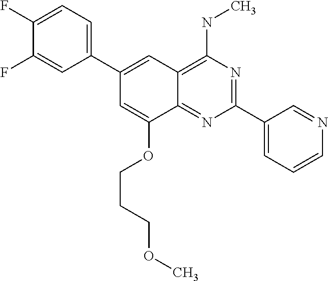 [SMILES: CNc1nc(-c2cccnc2)nc2c(OCCCOC)cc(-c3ccc(F)c(F)c3)cc12] |
| PFP-00731 | 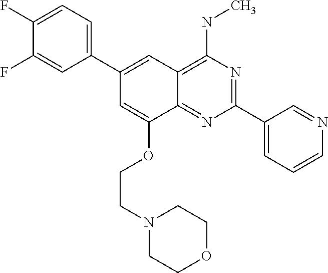 [SMILES: CNc1nc(-c2cccnc2)nc2c(OCCN3CCOCC3)cc(-c3ccc(F)c(F)c3)cc12] |
| PFP-00732 | 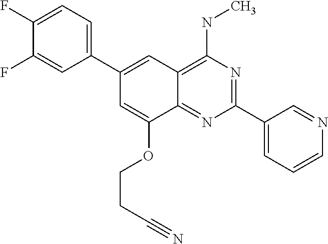 [SMILES: CNc1nc(-c2cccnc2)nc2c(OCCC#N)cc(-c3ccc(F)c(F)c3)cc12] |
| PFP-00733 | 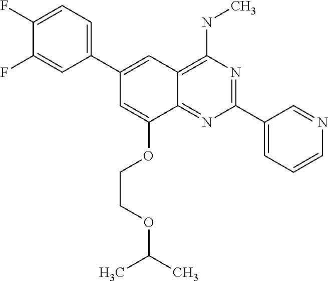 [SMILES: CNc1nc(-c2cccnc2)nc2c(OCCOC(C)C)cc(-c3ccc(F)c(F)c3)cc12] |
| PFP-00734 | 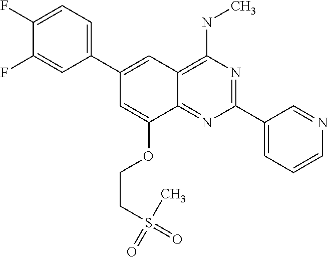 [SMILES: CNc1nc(-c2cccnc2)nc2c(OCCS(C)(=O)=O)cc(-c3ccc(F)c(F)c3)cc12] |
| PFP-00735 | 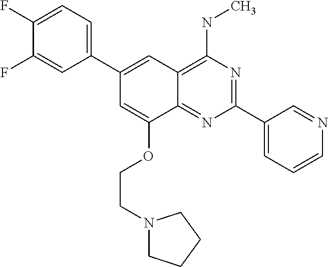 [SMILES: CNc1nc(-c2cccnc2)nc2c(OCCN3CCCC3)cc(-c3ccc(F)c(F)c3)cc12] |
| PFP-00736 | 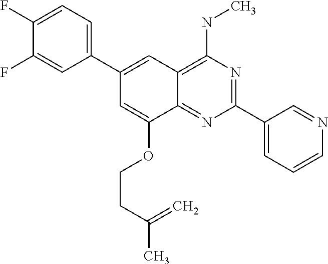 [SMILES: C=C(C)CCOc1cc(-c2ccc(F)c(F)c2)cc2c(NC)nc(-c3cccnc3)nc12] |
| PFP-00737 | 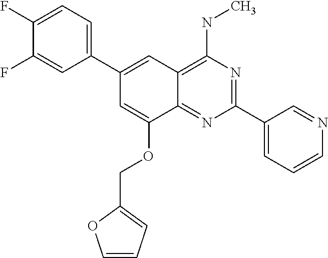 [SMILES: CNc1nc(-c2cccnc2)nc2c(OCc3ccco3)cc(-c3ccc(F)c(F)c3)cc12] |
| PFP-00738 | 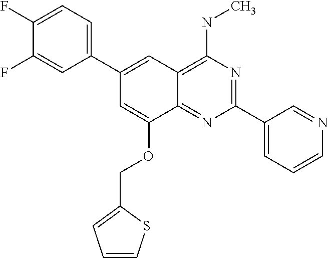 [SMILES: CNc1nc(-c2cccnc2)nc2c(OCc3cccs3)cc(-c3ccc(F)c(F)c3)cc12] |
| PFP-00739 | 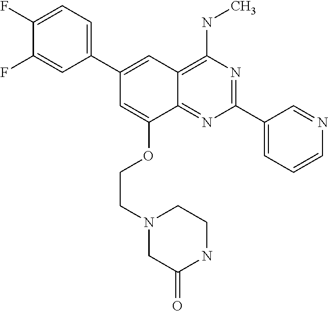 [SMILES: CNc1nc(-c2cccnc2)nc2c(OCCN3CCNC(=O)C3)cc(-c3ccc(F)c(F)c3)cc12] |
| PFP-00740 | 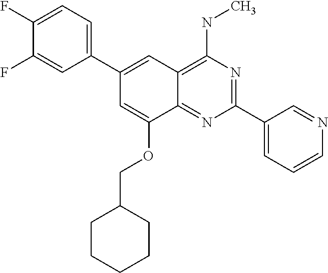 [SMILES: CNc1nc(-c2cccnc2)nc2c(OCC3CCCCC3)cc(-c3ccc(F)c(F)c3)cc12] |
| PFP-00741 | 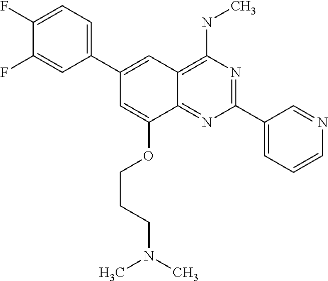 [SMILES: CNc1nc(-c2cccnc2)nc2c(OCCCN(C)C)cc(-c3ccc(F)c(F)c3)cc12] |
| PFP-00742 | 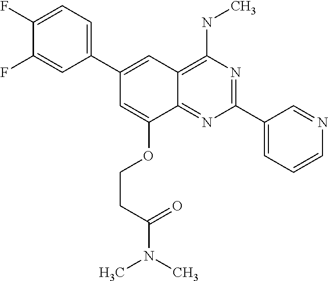 [SMILES: CNc1nc(-c2cccnc2)nc2c(OCCC(=O)N(C)C)cc(-c3ccc(F)c(F)c3)cc12] |
| PFP-00743 | 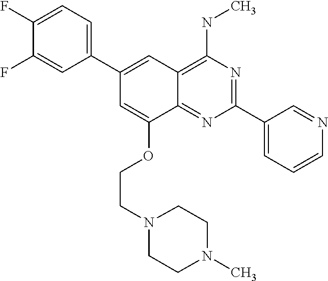 [SMILES: CNc1nc(-c2cccnc2)nc2c(OCCN3CCN(C)CC3)cc(-c3ccc(F)c(F)c3)cc12] |
| PFP-00744 | 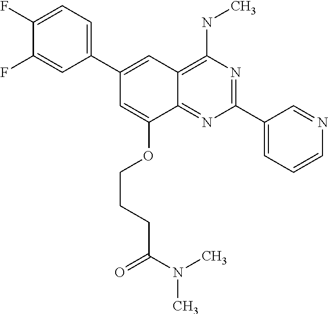 [SMILES: CNc1nc(-c2cccnc2)nc2c(OCCCC(=O)N(C)C)cc(-c3ccc(F)c(F)c3)cc12] |
| PFP-00745 | 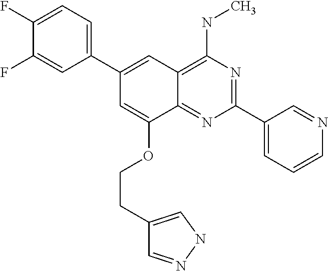 [SMILES: CNc1nc(-c2cccnc2)nc2c(OCCc3cn[nH]c3)cc(-c3ccc(F)c(F)c3)cc12] |
| PFP-00746 | 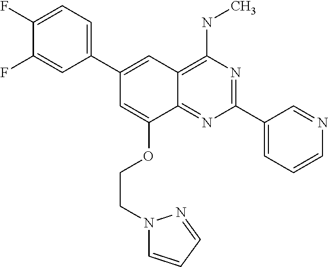 [SMILES: CNc1nc(-c2cccnc2)nc2c(OCCn3cccn3)cc(-c3ccc(F)c(F)c3)cc12] |
| PFP-00747 | 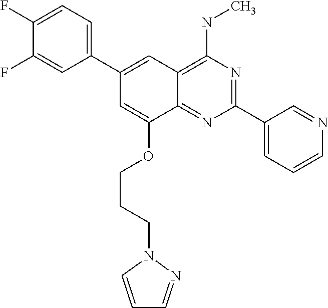 [SMILES: CNc1nc(-c2cccnc2)nc2c(OCCCn3cccn3)cc(-c3ccc(F)c(F)c3)cc12] |
| PFP-00748 | 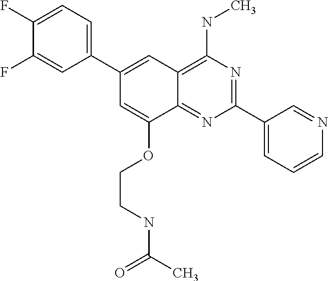 [SMILES: CNc1nc(-c2cccnc2)nc2c(OCCNC(C)=O)cc(-c3ccc(F)c(F)c3)cc12] |
| PFP-00749 | 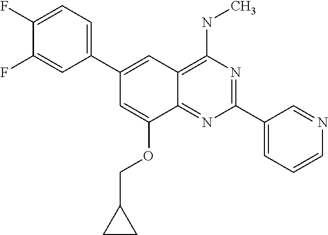 [SMILES: CNc1nc(-c2cccnc2)nc2c(OCC3CC3)cc(-c3ccc(F)c(F)c3)cc12] |
| PFP-00750 | 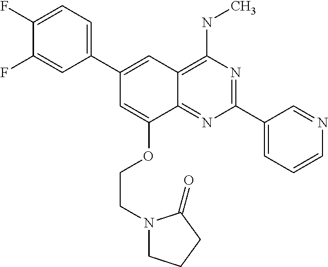 [SMILES: CNc1nc(-c2cccnc2)nc2c(OCCN3CCCC3=O)cc(-c3ccc(F)c(F)c3)cc12] |
| PFP-00751 | 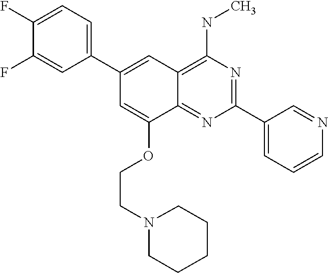 [SMILES: CNc1nc(-c2cccnc2)nc2c(OCCN3CCCCC3)cc(-c3ccc(F)c(F)c3)cc12] |
| PFP-00752 | 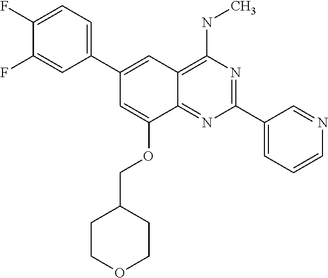 [SMILES: CNc1nc(-c2cccnc2)nc2c(OCC3CCOCC3)cc(-c3ccc(F)c(F)c3)cc12] |
| PFP-00753 | 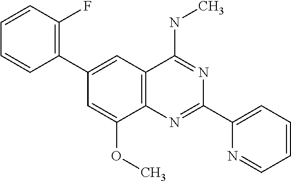 [SMILES: CNc1nc(-c2ccccn2)nc2c(OC)cc(-c3ccccc3F)cc12] |
| PFP-00755 | 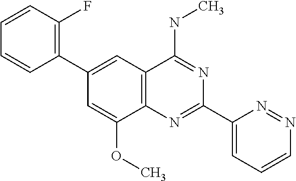 [SMILES: CNc1nc(-c2cccnn2)nc2c(OC)cc(-c3ccccc3F)cc12] |
| PFP-00756 | 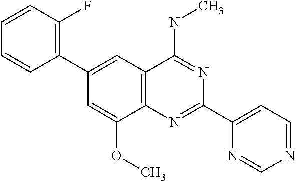 [SMILES: CNc1nc(-c2ccncn2)nc2c(OC)cc(-c3ccccc3F)cc12] |
| PFP-00757 | 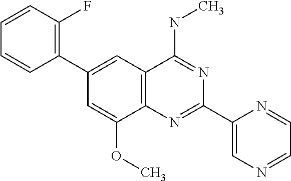 [SMILES: CNc1nc(-c2cnccn2)nc2c(OC)cc(-c3ccccc3F)cc12] |
| PFP-00758 | 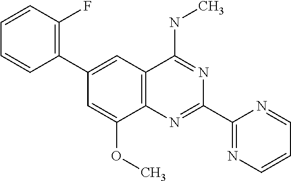 [SMILES: CNc1nc(-c2ncccn2)nc2c(OC)cc(-c3ccccc3F)cc12] |
| PFP-00759 | 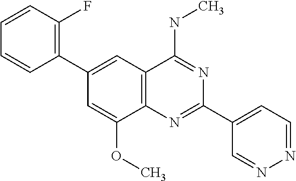 [SMILES: CNc1nc(-c2ccnnc2)nc2c(OC)cc(-c3ccccc3F)cc12] |
| PFP-00760 | 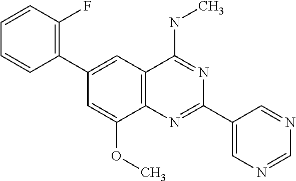 [SMILES: CNc1nc(-c2cncnc2)nc2c(OC)cc(-c3ccccc3F)cc12] |
| PFP-00761 | 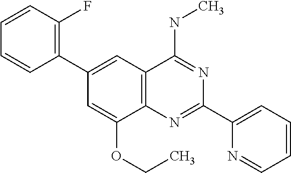 [SMILES: CCOc1cc(-c2ccccc2F)cc2c(NC)nc(-c3ccccn3)nc12] |
| PFP-00763 | 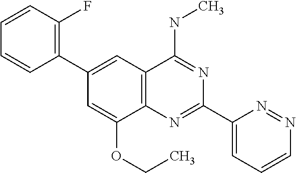 [SMILES: CCOc1cc(-c2ccccc2F)cc2c(NC)nc(-c3cccnn3)nc12] |
| PFP-00764 | 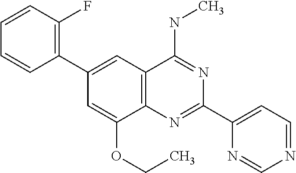 [SMILES: CCOc1cc(-c2ccccc2F)cc2c(NC)nc(-c3ccncn3)nc12] |
| PFP-00765 | 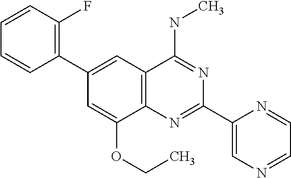 [SMILES: CCOc1cc(-c2ccccc2F)cc2c(NC)nc(-c3cnccn3)nc12] |
| PFP-00766 | 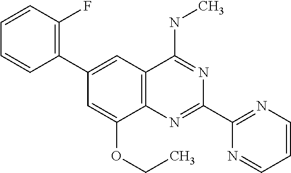 [SMILES: CCOc1cc(-c2ccccc2F)cc2c(NC)nc(-c3ncccn3)nc12] |
| PFP-00767 | 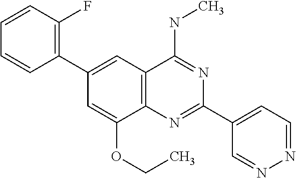 [SMILES: CCOc1cc(-c2ccccc2F)cc2c(NC)nc(-c3ccnnc3)nc12] |
| PFP-00768 | 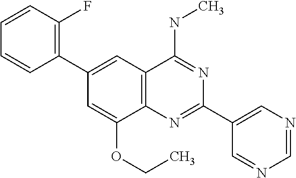 [SMILES: CCOc1cc(-c2ccccc2F)cc2c(NC)nc(-c3cncnc3)nc12] |
| PFP-00769 | 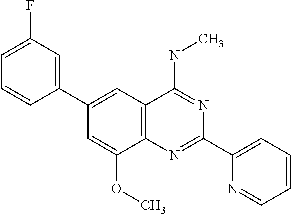 [SMILES: CNc1nc(-c2ccccn2)nc2c(OC)cc(-c3cccc(F)c3)cc12] |
| PFP-00771 | 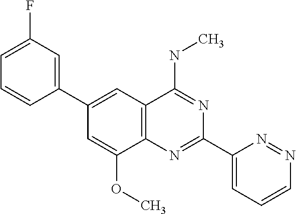 [SMILES: CNc1nc(-c2cccnn2)nc2c(OC)cc(-c3cccc(F)c3)cc12] |
| PFP-00772 | 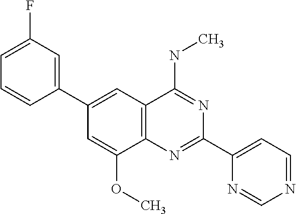 [SMILES: CNc1nc(-c2ccncn2)nc2c(OC)cc(-c3cccc(F)c3)cc12] |
| PFP-00773 | 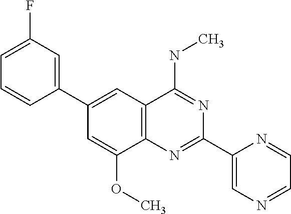 [SMILES: CNc1nc(-c2cnccn2)nc2c(OC)cc(-c3cccc(F)c3)cc12] |
| PFP-00774 | 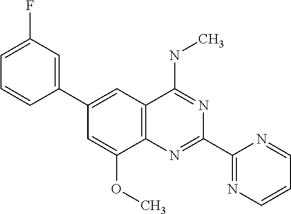 [SMILES: CNc1nc(-c2ncccn2)nc2c(OC)cc(-c3cccc(F)c3)cc12] |
| PFP-00775 | 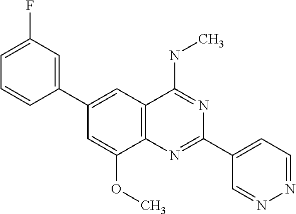 [SMILES: CNc1nc(-c2ccnnc2)nc2c(OC)cc(-c3cccc(F)c3)cc12] |
| PFP-00776 | 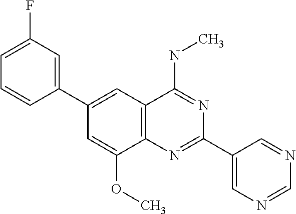 [SMILES: CNc1nc(-c2cncnc2)nc2c(OC)cc(-c3cccc(F)c3)cc12] |
| PFP-00777 | 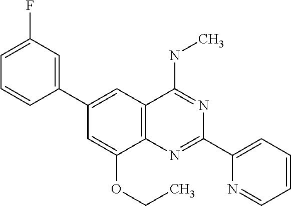 [SMILES: CCOc1cc(-c2cccc(F)c2)cc2c(NC)nc(-c3ccccn3)nc12] |
| PFP-00779 | 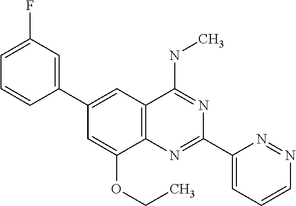 [SMILES: CCOc1cc(-c2cccc(F)c2)cc2c(NC)nc(-c3cccnn3)nc12] |
| PFP-00780 | 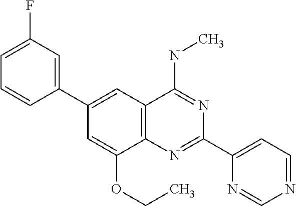 [SMILES: CCOc1cc(-c2cccc(F)c2)cc2c(NC)nc(-c3ccncn3)nc12] |
| PFP-00781 | 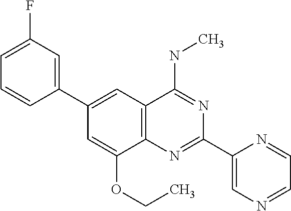 [SMILES: CCOc1cc(-c2cccc(F)c2)cc2c(NC)nc(-c3cnccn3)nc12] |
| PFP-00782 | 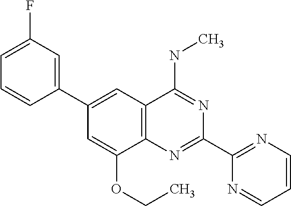 [SMILES: CCOc1cc(-c2cccc(F)c2)cc2c(NC)nc(-c3ncccn3)nc12] |
| PFP-00783 | 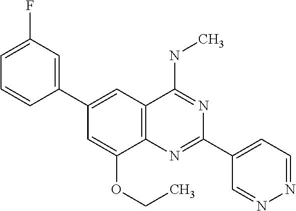 [SMILES: CCOc1cc(-c2cccc(F)c2)cc2c(NC)nc(-c3ccnnc3)nc12] |
| PFP-00784 | 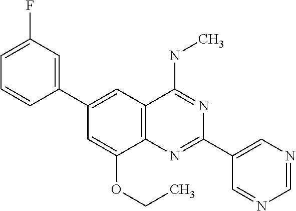 [SMILES: CCOc1cc(-c2cccc(F)c2)cc2c(NC)nc(-c3cncnc3)nc12] |
| PFP-00785 | 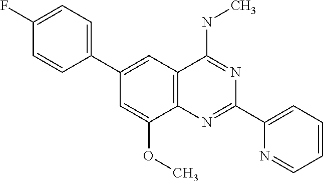 [SMILES: CNc1nc(-c2ccccn2)nc2c(OC)cc(-c3ccc(F)cc3)cc12] |
| PFP-00787 | 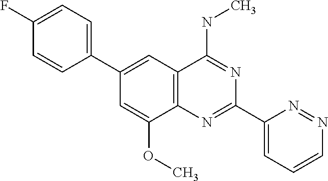 [SMILES: CNc1nc(-c2cccnn2)nc2c(OC)cc(-c3ccc(F)cc3)cc12] |
| PFP-00788 | 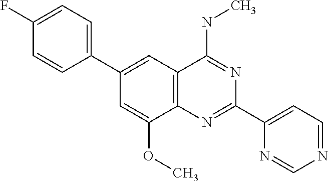 [SMILES: CNc1nc(-c2ccncn2)nc2c(OC)cc(-c3ccc(F)cc3)cc12] |
| PFP-00789 | 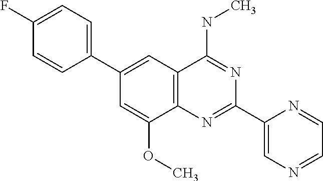 [SMILES: CNc1nc(-c2cnccn2)nc2c(OC)cc(-c3ccc(F)cc3)cc12] |
| PFP-00790 | 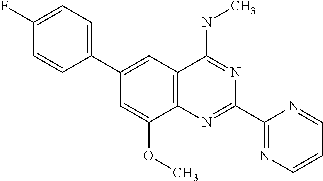 [SMILES: CNc1nc(-c2ncccn2)nc2c(OC)cc(-c3ccc(F)cc3)cc12] |
| PFP-00791 | 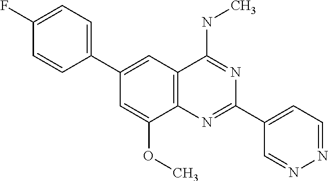 [SMILES: CNc1nc(-c2ccnnc2)nc2c(OC)cc(-c3ccc(F)cc3)cc12] |
| PFP-00792 | 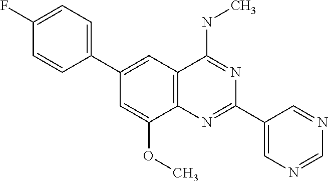 [SMILES: CNc1nc(-c2cncnc2)nc2c(OC)cc(-c3ccc(F)cc3)cc12] |
| PFP-00793 | 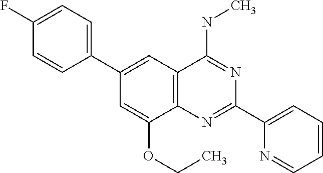 [SMILES: CCOc1cc(-c2ccc(F)cc2)cc2c(NC)nc(-c3ccccn3)nc12] |
| PFP-00795 | 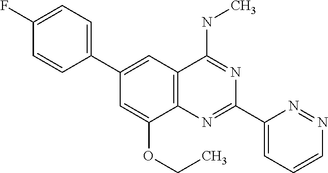 [SMILES: CCOc1cc(-c2ccc(F)cc2)cc2c(NC)nc(-c3cccnn3)nc12] |
| PFP-00796 | 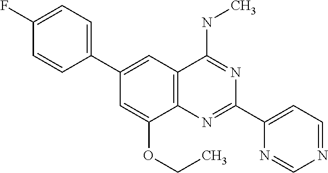 [SMILES: CCOc1cc(-c2ccc(F)cc2)cc2c(NC)nc(-c3ccncn3)nc12] |
| PFP-00797 | 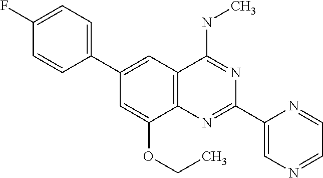 [SMILES: CCOc1cc(-c2ccc(F)cc2)cc2c(NC)nc(-c3cnccn3)nc12] |
| PFP-00798 | 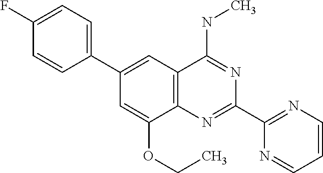 [SMILES: CCOc1cc(-c2ccc(F)cc2)cc2c(NC)nc(-c3ncccn3)nc12] |
| PFP-00799 | 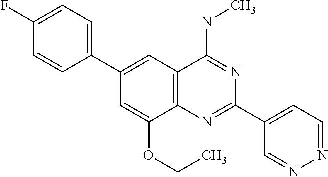 [SMILES: CCOc1cc(-c2ccc(F)cc2)cc2c(NC)nc(-c3ccnnc3)nc12] |
| PFP-00800 | 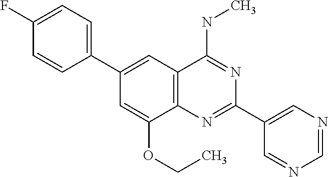 [SMILES: CCOc1cc(-c2ccc(F)cc2)cc2c(NC)nc(-c3cncnc3)nc12] |
| PFP-00801 | 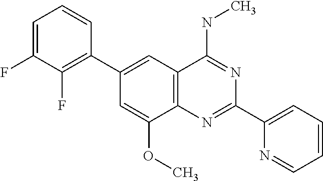 [SMILES: CNc1nc(-c2ccccn2)nc2c(OC)cc(-c3cccc(F)c3F)cc12] |
| PFP-00803 | 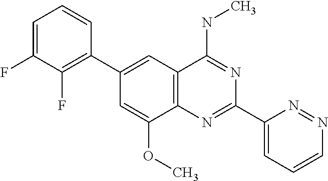 [SMILES: CNc1nc(-c2cccnn2)nc2c(OC)cc(-c3cccc(F)c3F)cc12] |
| PFP-00804 | 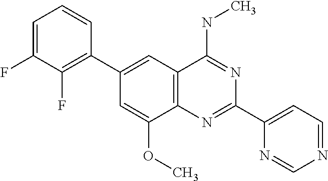 [SMILES: CNc1nc(-c2ccncn2)nc2c(OC)cc(-c3cccc(F)c3F)cc12] |
| PFP-00805 | 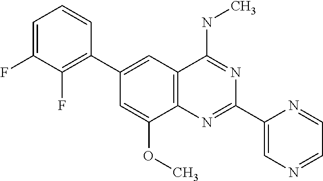 [SMILES: CNc1nc(-c2cnccn2)nc2c(OC)cc(-c3cccc(F)c3F)cc12] |
| PFP-00806 | 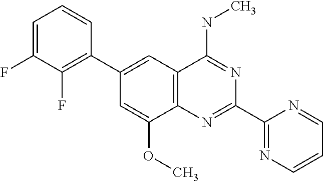 [SMILES: CNc1nc(-c2ncccn2)nc2c(OC)cc(-c3cccc(F)c3F)cc12] |
| PFP-00807 | 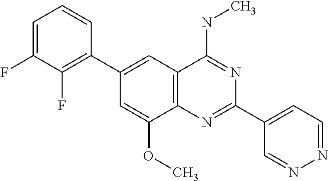 [SMILES: CNc1nc(-c2ccnnc2)nc2c(OC)cc(-c3cccc(F)c3F)cc12] |
| PFP-00808 | 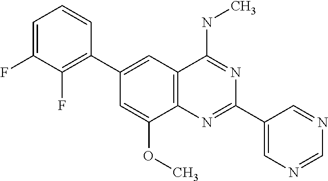 [SMILES: CNc1nc(-c2cncnc2)nc2c(OC)cc(-c3cccc(F)c3F)cc12] |
| PFP-00809 | 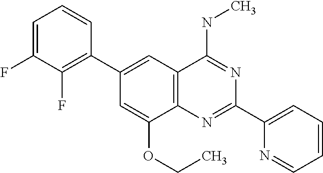 [SMILES: CCOc1cc(-c2cccc(F)c2F)cc2c(NC)nc(-c3ccccn3)nc12] |
| PFP-00811 | 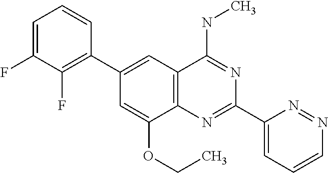 [SMILES: CCOc1cc(-c2cccc(F)c2F)cc2c(NC)nc(-c3cccnn3)nc12] |
| PFP-00812 | 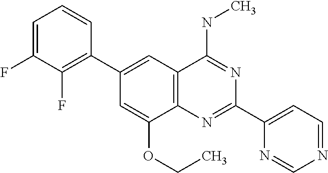 [SMILES: CCOc1cc(-c2cccc(F)c2F)cc2c(NC)nc(-c3ccncn3)nc12] |
| PFP-00813 | 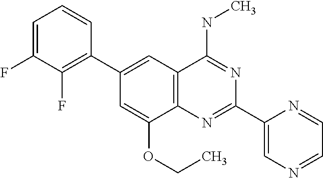 [SMILES: CCOc1cc(-c2cccc(F)c2F)cc2c(NC)nc(-c3cnccn3)nc12] |
| PFP-00814 | 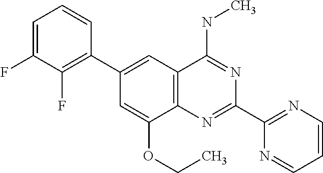 [SMILES: CCOc1cc(-c2cccc(F)c2F)cc2c(NC)nc(-c3ncccn3)nc12] |
| PFP-00815 | 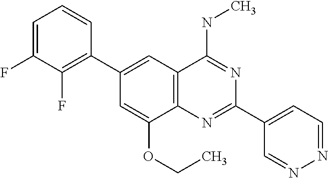 [SMILES: CCOc1cc(-c2cccc(F)c2F)cc2c(NC)nc(-c3ccnnc3)nc12] |
| PFP-00816 | 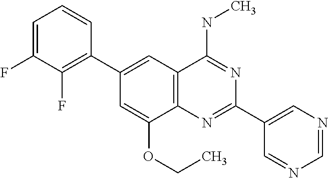 [SMILES: CCOc1cc(-c2cccc(F)c2F)cc2c(NC)nc(-c3cncnc3)nc12] |
| PFP-00817 | 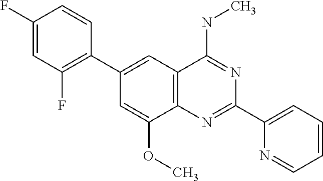 [SMILES: CNc1nc(-c2ccccn2)nc2c(OC)cc(-c3ccc(F)cc3F)cc12] |
| PFP-00819 | 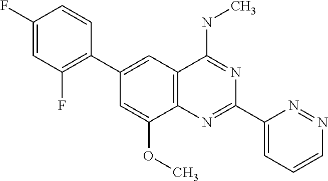 [SMILES: CNc1nc(-c2cccnn2)nc2c(OC)cc(-c3ccc(F)cc3F)cc12] |
| PFP-00820 | 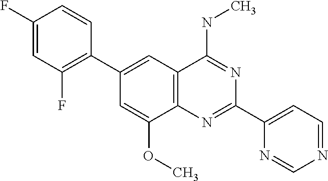 [SMILES: CNc1nc(-c2ccncn2)nc2c(OC)cc(-c3ccc(F)cc3F)cc12] |
| PFP-00821 | 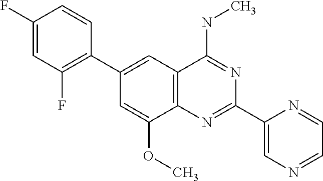 [SMILES: CNc1nc(-c2cnccn2)nc2c(OC)cc(-c3ccc(F)cc3F)cc12] |
| PFP-00822 | 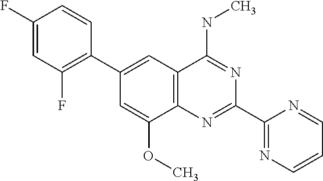 [SMILES: CNc1nc(-c2ncccn2)nc2c(OC)cc(-c3ccc(F)cc3F)cc12] |
| PFP-00823 | 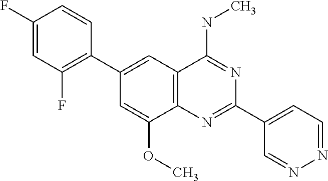 [SMILES: CNc1nc(-c2ccnnc2)nc2c(OC)cc(-c3ccc(F)cc3F)cc12] |
| PFP-00824 | 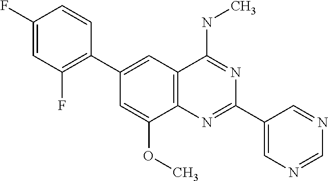 [SMILES: CNc1nc(-c2cncnc2)nc2c(OC)cc(-c3ccc(F)cc3F)cc12] |
| PFP-00825 | 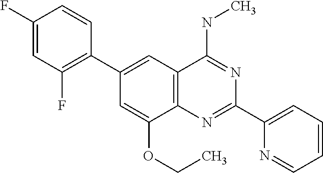 [SMILES: CCOc1cc(-c2ccc(F)cc2F)cc2c(NC)nc(-c3ccccn3)nc12] |
| PFP-00827 | 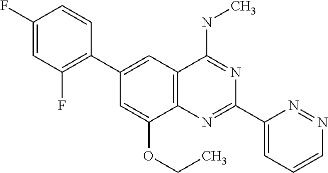 [SMILES: CCOc1cc(-c2ccc(F)cc2F)cc2c(NC)nc(-c3cccnn3)nc12] |
| PFP-00828 | 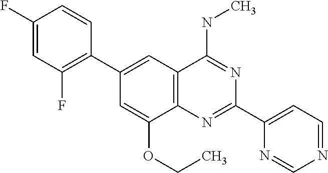 [SMILES: CCOc1cc(-c2ccc(F)cc2F)cc2c(NC)nc(-c3ccncn3)nc12] |
| PFP-00829 | 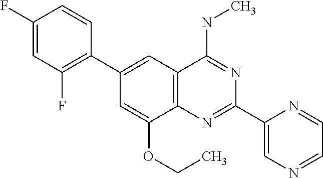 [SMILES: CCOc1cc(-c2ccc(F)cc2F)cc2c(NC)nc(-c3cnccn3)nc12] |
| PFP-00830 | 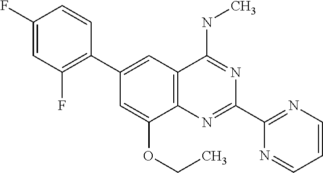 [SMILES: CCOc1cc(-c2ccc(F)cc2F)cc2c(NC)nc(-c3ncccn3)nc12] |
| PFP-00831 | 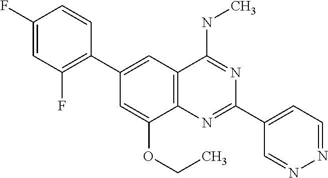 [SMILES: CCOc1cc(-c2ccc(F)cc2F)cc2c(NC)nc(-c3ccnnc3)nc12] |
| PFP-00832 | 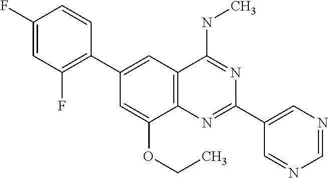 [SMILES: CCOc1cc(-c2ccc(F)cc2F)cc2c(NC)nc(-c3cncnc3)nc12] |
| PFP-00833 | 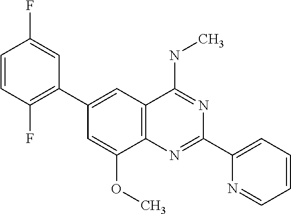 [SMILES: CNc1nc(-c2ccccn2)nc2c(OC)cc(-c3cc(F)ccc3F)cc12] |
| PFP-00835 | 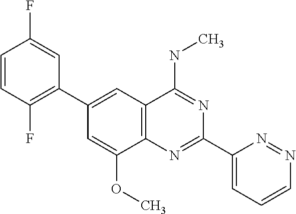 [SMILES: CNc1nc(-c2cccnn2)nc2c(OC)cc(-c3cc(F)ccc3F)cc12] |
| PFP-00836 | 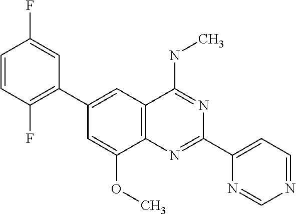 [SMILES: CNc1nc(-c2ccncn2)nc2c(OC)cc(-c3cc(F)ccc3F)cc12] |
| PFP-00837 | 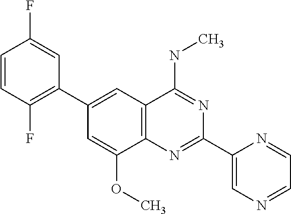 [SMILES: CNc1nc(-c2cnccn2)nc2c(OC)cc(-c3cc(F)ccc3F)cc12] |
| PFP-00838 | 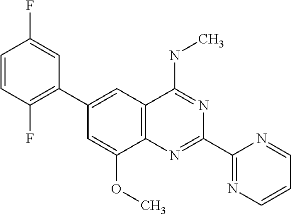 [SMILES: CNc1nc(-c2ncccn2)nc2c(OC)cc(-c3cc(F)ccc3F)cc12] |
| PFP-00839 | 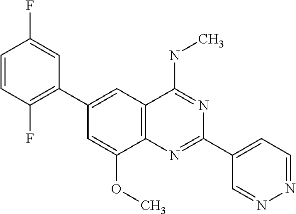 [SMILES: CNc1nc(-c2ccnnc2)nc2c(OC)cc(-c3cc(F)ccc3F)cc12] |
| PFP-00840 | 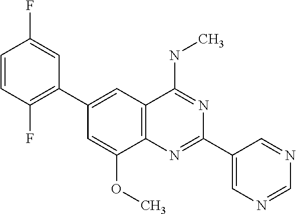 [SMILES: CNc1nc(-c2cncnc2)nc2c(OC)cc(-c3cc(F)ccc3F)cc12] |
| PFP-00841 | 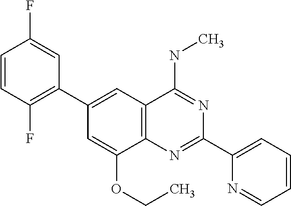 [SMILES: CCOc1cc(-c2cc(F)ccc2F)cc2c(NC)nc(-c3ccccn3)nc12] |
| PFP-00843 | 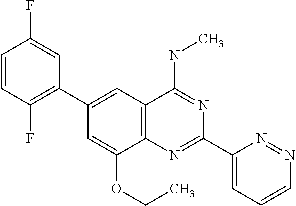 [SMILES: CCOc1cc(-c2cc(F)ccc2F)cc2c(NC)nc(-c3cccnn3)nc12] |
| PFP-00844 | 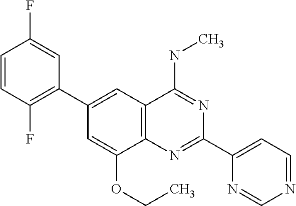 [SMILES: CCOc1cc(-c2cc(F)ccc2F)cc2c(NC)nc(-c3ccncn3)nc12] |
| PFP-00845 | 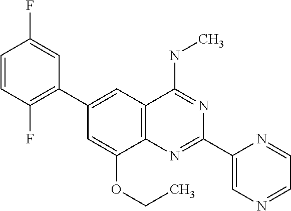 [SMILES: CCOc1cc(-c2cc(F)ccc2F)cc2c(NC)nc(-c3cnccn3)nc12] |
| PFP-00846 | 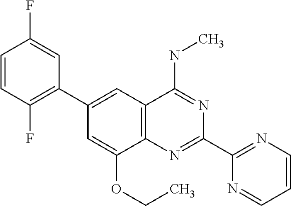 [SMILES: CCOc1cc(-c2cc(F)ccc2F)cc2c(NC)nc(-c3ncccn3)nc12] |
| PFP-00847 | 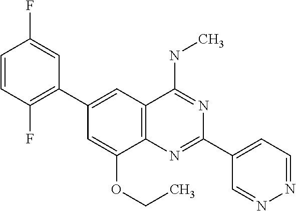 [SMILES: CCOc1cc(-c2cc(F)ccc2F)cc2c(NC)nc(-c3ccnnc3)nc12] |
| PFP-00848 | 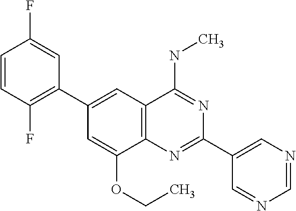 [SMILES: CCOc1cc(-c2cc(F)ccc2F)cc2c(NC)nc(-c3cncnc3)nc12] |
| PFP-00849 | 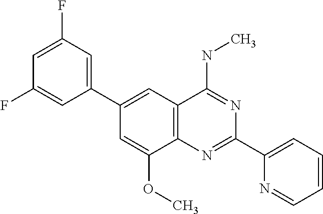 [SMILES: CNc1nc(-c2ccccn2)nc2c(OC)cc(-c3cc(F)cc(F)c3)cc12] |
| PFP-00851 | 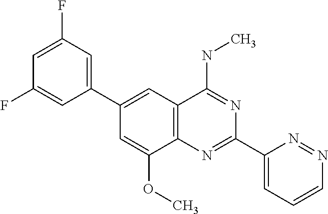 [SMILES: CNc1nc(-c2cccnn2)nc2c(OC)cc(-c3cc(F)cc(F)c3)cc12] |
| PFP-00852 | 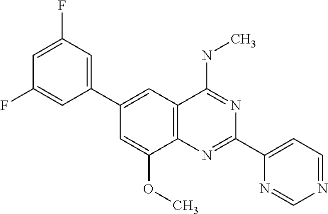 [SMILES: CNc1nc(-c2ccncn2)nc2c(OC)cc(-c3cc(F)cc(F)c3)cc12] |
| PFP-00853 | 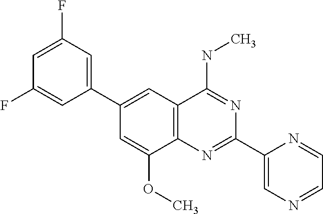 [SMILES: CNc1nc(-c2cnccn2)nc2c(OC)cc(-c3cc(F)cc(F)c3)cc12] |
| PFP-00854 | 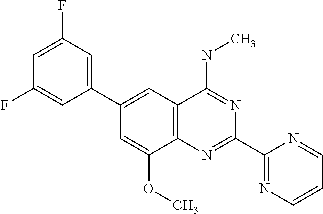 [SMILES: CNc1nc(-c2ncccn2)nc2c(OC)cc(-c3cc(F)cc(F)c3)cc12] |
| PFP-00855 | 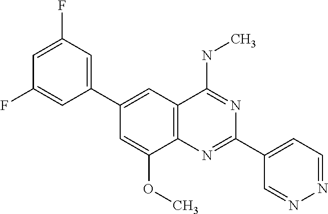 [SMILES: CNc1nc(-c2ccnnc2)nc2c(OC)cc(-c3cc(F)cc(F)c3)cc12] |
| PFP-00856 | 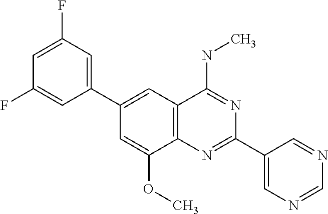 [SMILES: CNc1nc(-c2cncnc2)nc2c(OC)cc(-c3cc(F)cc(F)c3)cc12] |
| PFP-00857 | 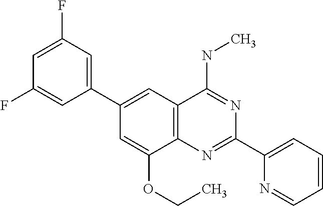 [SMILES: CCOc1cc(-c2cc(F)cc(F)c2)cc2c(NC)nc(-c3ccccn3)nc12] |
| PFP-00859 | 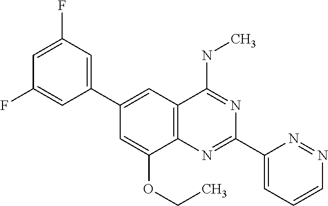 [SMILES: CCOc1cc(-c2cc(F)cc(F)c2)cc2c(NC)nc(-c3cccnn3)nc12] |
| PFP-00860 | 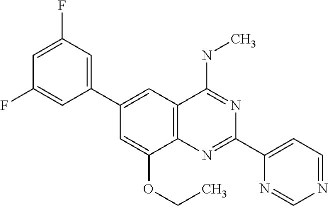 [SMILES: CCOc1cc(-c2cc(F)cc(F)c2)cc2c(NC)nc(-c3ccncn3)nc12] |
| PFP-00861 | 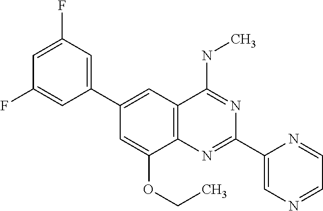 [SMILES: CCOc1cc(-c2cc(F)cc(F)c2)cc2c(NC)nc(-c3cnccn3)nc12] |
| PFP-00862 | 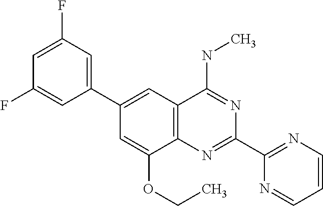 [SMILES: CCOc1cc(-c2cc(F)cc(F)c2)cc2c(NC)nc(-c3ncccn3)nc12] |
| PFP-00863 | 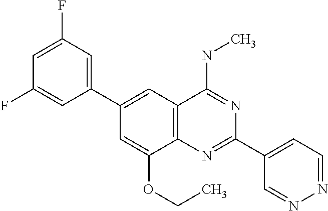 [SMILES: CCOc1cc(-c2cc(F)cc(F)c2)cc2c(NC)nc(-c3ccnnc3)nc12] |
| PFP-00864 | 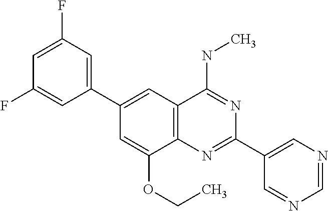 [SMILES: CCOc1cc(-c2cc(F)cc(F)c2)cc2c(NC)nc(-c3cncnc3)nc12] |

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

1. A compound of formula (I): 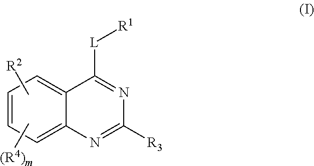
or a salt thereof,
wherein:
m is 0 or 1;
L is a direct bond or NR⁶;
R¹ is hydrogen, C₁-C₈ alkyl, halo C₁-C₈ alkyl, C₁-C₈ alkoxy C₁-C₈ alkyl, hydroxy C₁-C₈ alkyl, amino C₁-C₈ alkyl, oxazolyl, thiazolyl, isoxazolyl, pyridyl, pyrrolopyridyl, oxadiazolyl C₁-C₈ alkyl, pyridyl C₁-C₈ alkyl, oxazolyl C₁-C₈ alkyl, phenyl C₁-C₈ alkyl, —C(O)R^{e}, pyrrolidinyl, azetidinyl, indolinyl, piperidinyl, morpholinyl, piperazinyl, phenyl, C₃-C₈ cycloalkyl, C₃-C₈ cycloalkyl C₁-C₈ alkyl, benzoxazolyl, each of which is optionally substituted with 1-2 R⁷;
R² is C₁-C₈ alkoxy, benzodioxolyl, piperazinyl, halo, phenyl, tetrahydronaphtyl, furyl, oxazolyl, thiazolyl, thiadiazolyl, pyridyl, pyrimidinyl, indolyl, indazolyl, dihydroindazolyl, tetrahydroisoquinolinyl, tetrahydroquinolinyl, dihydrobenzoimidazolyl, dihydrobenzoxazolyl, benzothiazolyl, dihydrobenzothiazolyl, benzothienyl, dihydroisoquinolinyl, isoquinolinyl, benzofuryl, dihydrobenzofuryl, benzodioxolyl, dihydrobenzoxazinyl, dihydrobenzodioxepinyl, tetrahydrobenzoxazepinyl, isoindolinyl, indolinyl, thienyl or dihydrobenzodioxinyl, each of which is optionally substituted with 1-3 R⁹;
R³ is pyridyl, pyrimidinyl, pyrazinyl or pyridazinyl, each of which is optionally substituted with C₁-C₈ alkyl, C₁-C₈ alkoxy, halo, halo C₁-C₈ alkyl, halo C₁-C₈ alkoxy, cyano or —OR^{d};
R⁴ is hydrogen, C₁-C₈ alkyl, C₁-C₈ alkoxy, halo, halo C₁-C₈ alkyl or halo C₁-C₈ alkoxy, each of which is optionally substituted with R¹⁰;
R⁶ is hydrogen or C₁-C₈ alkyl;
R⁷ is C₁-C₈ alkyl, C₂-C₈ alkenyl, C₂-C₈ alkynyl, aryl, heteroaryl, cyclyl, heterocyclyl, arylalkyl, heteroarylalkyl, cyclylalkyl, heterocyclylalkyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with R¹²;
R⁹ is C₁-C₈ alkyl, C₁-C₈ alkoxy, phenyl, pyrazolyl, dihydrobenzoxazolyl, oxazolyl, tetrazolyl, imidazolyl, thiazolyl, C₃-C₈ cycloalkyl, oxetanyl, pyrrolidinyl, morpholinyl, halo, halo C₁-C₈ alkyl, halo C₁-C₈ alkoxy, hydroxy C₁-C₈ alkyl, oxo, cyano, nitro, —C(O)OR^{a}, —C(O)NR^{b}R^{b′}, —NR^{c}C(O)R^{c′}, —NR^{b}R^{b′}, —OR^{d}, —SR^{d′}, —C(O)R^{e} or —S(O)_{q}R^{f}, each of which is optionally substituted with 1-2 R¹²;
R¹⁰ is C₁-C₈ alkoxy, C₂-C₈ alkenyl, C₃-C₈ cycloalkyl, furyl, thienyl, pyrazolyl, morpholinyl, piperazinyl, pyrrolidinyl, piperidinyl, tetrahydropyranyl, cyano, —C(O)NR^{b}R^{b′}, —NR^{c}C(O)R^{c′}, —NR^{b}R^{b′} or —S(O)_{q}R^{f}, each of which is optionally substituted with R¹²;
R¹² is C₁-C₈ alkyl, C₂-C₈ alkenyl, C₂-C₈ alkynyl, aryl, heteroaryl, cyclyl, heterocyclyl, arylalkyl, heteroarylalkyl, cyclylalkyl, heterocyclylalkyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f},each of which is optionally substituted with 1-3 R¹³;
R¹³ is independently C₁-C₈ alkyl, haloalkyl, halo, heterocyclyl, cyclyl, oxo or —C(Y)NR^{b}R^{b′};
each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl; and
q is 1 or 2.

2. The compound according to claim 1 represented by general formula (I) or a salt thereof,
wherein:
if R³ is 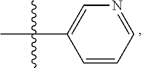 [SMILES: *c1cccnc1]
L is NR⁶, R¹ is benzyl, R⁶ is hydrogen, and R⁴ is hydrogen, then R² is not halo or methoxy;
if R³ is 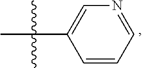 [SMILES: *c1cccnc1]
L is NR⁶, R¹ is phenyl, R⁶ is methyl, and R⁴ is hydrogen, then R² is not halo;
if R³ is 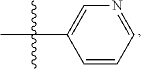 [SMILES: *c1cccnc1 ; ","]
L is NR⁶, R¹ is para-trifluoromethyl-phenyl, R⁶ is hydrogen, and R⁴ is hydrogen, then R² is not 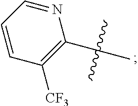 [SMILES: *c1ncccc1C(F)(F)F ; ";"]
if R³ is 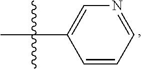 [SMILES: *c1cccnc1 ; ","]
L is NR⁶, R¹ is indolinyl, R⁶ is hydrogen, and R⁴ is hydrogen, then R² is not chloro; and
if R³ is 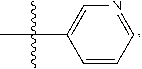 [SMILES: *c1cccnc1 ; ","]
L is NR⁶, R¹ is dimethylaminomethyl, R⁶ is hydrogen, and R⁴ is methoxy, then R² is not methoxy.

3. The compound according to claim 2 represented by general formula (I) or a salt thereof, provided the compounds in Table X are excluded.

4. The compound according to claim 1, represented by general formula (I) or a salt thereof,
wherein:
R¹ is C₃-C₈ cycloalkyl, oxazolyl, thiazolyl, isoxazolyl, pyridyl, pyrrolopyridyl, indolinyl, phenyl or benzoxazolyl, each of which is optionally substituted with 1-2 R⁷;
R² is C₁-C₈ alkoxy, piperazinyl, halo or pyrimidinyl, each of which is optionally substituted with 1-3 R⁹;
R³ is pyridyl (e.g, 3-pyridyl);
R⁴ is hydrogen;
R⁶ is hydrogen;
R⁷ is C₁-C₈ alkyl, C₁-C₈ alkoxy, halo, halo C₁-C alkyl, cyano, nitro or —C(O)NR^{b}R^{b′} or —NR^{c}C(O)R^{c′};
R⁹ is C₁-C₈ alkyl, C₁-C₈ alkoxy, halo, cyano, nitro, —C(O)NR^{b}R^{b′} or —NR^{c}C(O)R^{c′}, —NR^{b}R^{b′};
each R^{a}, R^{b}, R^{b′}, R^{c}, and R^{c′} is independently hydrogen, C₁-C₈ alkyl or C₁-C₈ alkoxy; and
q is 1 or 2.

5. The compound according to claim 1, represented by general formula (I) or a salt thereof,
wherein:
R¹ is C₁-C₈ alkyl, phenyl or pyridyl C₁-C₈ alkyl, each of which is optionally substituted with 1-2 R⁷;
R² is C₁-C₈ alkoxy or phenyl, each of which is optionally substituted with 1-3 R⁹;
R³ is pyrimidinyl, pyrazinyl or pyridazinyl;
R⁴ is hydrogen or C₁-C₈ alkoxy;
R⁶ is hydrogen;
R⁷ is C₁-C₈ alkyl or —C(O)NH₂;
R⁹ is halo; and q is 1 or 2.

6. The compound according to claim 1, represented by general formula (I) or a salt thereof,
wherein:
m is 0 or 1;
R¹ is hydrogen, C₁-C₈ alkyl, halo C₁-C₈ alkyl, C₁-C₈ alkoxy C₁-C₈ alkyl, hydroxyl C₁-C₈ alkyl, amino C₁-C₈ alkyl, oxazolyl, thiazolyl, isoxazolyl, pyridyl, pyrrolopyridyl, oxadiazolyl C₁-C₈ alkyl, pyridyl C₁-C₈ alkyl, oxazolyl C₁-C₈ alkyl, phenyl C₁-C₈ alkyl, —C(O)R^{e}, C₃-C₈ cycloalkyl, C₃-C₈ cycloalkyl C₁-C₈ alkyl, pyrrolidinyl, azetidinyl, indolinyl, piperidinyl, morpholinyl or piperazinyl, each of which is optionally substituted with 1-2 R⁷;
R² is phenyl, tetrahydronaphthyl, furyl, oxazolyl, thiazolyl, thiadiazolyl, pyridyl, pyrimidinyl, indolyl, indazolyl, dihydroindazolyl, tetrahydroisoquinolinyl, tetrahydroquinolinyl, dihydrobenzoimidazolyl, dihydrobenzoxazolyl, benzothiazolyl, dihydrobenzothiazolyl, benzothienyl, dihydroisoquinolinyl, isoquinolinyl, benzofuryl, dihydrobenzofuryl, benzodioxolyl, dihydrobenzoxazinyl, dihydrobenzodioxepinyl, tetrahydrobenzoxazepinyl, isoindolinyl, indolinyl, thienyl or dihydrobenzodioxinyl, each of which is optionally substituted with 1-3 R⁹;
R³ is pyridyl (e.g, 3-pyridyl), each of which is optionally substituted with C₁-C₈ alkyl, C₁-C₈ alkoxy, halo, halo C₁-C₈ alkyl, halo C₁-C₈ alkoxy, cyano or —OR^{d};
R⁴ is hydrogen, C₁-C₈ alkyl, C₁-C₈ alkoxy, halo, halo C₁-C₈ alkyl or halo C₁-C₈ alkoxy, each of which is optionally substituted with R¹⁰;
R⁶ is hydrogen or C₁-C₈ alkyl;
R⁷ is C₁-C₈ alkyl, C₁-C₈ alkoxy, pyrazolyl, pyridyl, C₃-C₈ cycloalkyl, halo, halo C₁-C₈ alkyl, halo C₁-C₈ alkoxy, C₁-C₈ alkylamino, di C₁-C₈ alkylamino, di C₁-C₈ alkyl amino C₁-C₈ alkyl, oxo, nitro, —C(O)NR^{b}R^{b′}, —NR^{c}C(O)R^{c′} or —C(O)R^{e}, each of which is optionally substituted with R¹²;
R⁹ is C₁-C₈ alkyl, C₁-C₈ alkoxy, phenyl, pyrazolyl, dihydrobenzoxazolyl,oxazolyl, tetrazolyl, imidazolyl, thiazolyl C₃-C₈ cycloalkyl, oxetanyl, pyrrolidinyl, morpholinyl, halo, halo C₁-C₈ alkyl, halo C₁-C₈ alkoxy, hydroxyl C₁-C₈ alkyl, oxo, cyano, nitro, —C(O)OR^{a}, —C(O)NR^{b}R^{b′}, —NR^{c}C(O)R^{c′}, —NR^{b}R^{b′}, —OR^{d}, —SR^{d′}, —C(O)R^{e} or —S(O)_{q}R^{f}, each of which is optionally substituted with 1-2 R¹²;
R¹⁰ is C₁-C₈ alkoxy, C₂-C₈ alkenyl, C₃-C₈ cycloalkyl, furyl, thienyl, pyrazolyl, morpholinyl, piperazinyl, pyrrolidinyl, piperidinyl, tetrahydropyranyl, cyano, —C(O)NR^{b}R^{b′}, —NR^{c}C(O)R^{c′}, —NR^{b}R^{b′} or —S(O)_{q}R^{f}, each of which is optionally substituted with R¹²;
R¹² is C₁-C₈ alkyl, C₁-C₈ alkoxy, halo, halo C₁-C₈ alkyl, silyl C₁-C₈ alkoxy, silyl C₁-C₈ alkoxy C₁-C₈ alkyl, oxo, thioxo, cyano, nitro, —C(O)OR^{a}, —C(O)NR^{b}R^{b′}, —NR^{c}C(O)R^{c′}, —NR^{b}R^{b′}, —OR^{d} or —C(O)R^{e};
each R^{a}, R^{b}, R^{b′}, R^{c}, R^{c′}, R^{d}, R^{d′}, R^{e} and R^{f} is independently hydrogen, amino, C₁-C₈ alkyl, C₁-C₈ alkoxy, C₂-C₈ alkenyl, C₁-C₈ alkoxy C₁-C₈ alkyl, C₃-C₈ cycloalkyl, tetrahydropyranyl, morpholinyl, thiadiazolyl or thiazolyl; and
q is 1 or 2.

7. The compound of claim 6, wherein R² is phenyl.

8. A compound of formula (II): 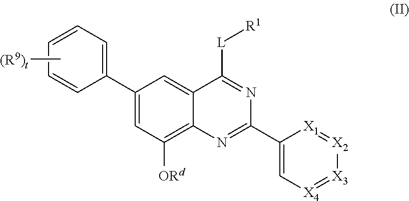
or a salt thereof,
wherein:
L is a direct bond or NR⁶;
one or two of X¹, X², X³, and X⁴ are N and the others are CH,
R¹ is hydrogen, C₁-C₈ alkyl, C₂-C₈ alkenyl, C₂-C₈ alkynyl, alkoxyalkyl, hydroxyalkyl, heteroaryl, heteroarylalkyl, arylalkyl, —C(Y)R^{e}, cyclyl,cyclylalkyl or heterocyclyl, each of which is optionally substituted with 1-3 R⁷;
R⁶ is hydrogen, C₁-C₈ alkyl, C₂-C₈ alkenyl, C₂-C₈ alkynyl, cyclyl or heterocyclyl, each of which is optionally substituted with 1-3 R¹¹;
R⁷ is C₁-C₈ alkyl, C₂-C₈ alkenyl, C₂-C₈ alkynyl, aryl, heteroaryl, cyclyl, heterocyclyl, arylalkyl, heteroarylalkyl, cyclylalkyl, heterocyclylalkyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′},—OR^{d}, —SR^{d′}, —C(Y)R^{e} or —S(O)_{q}R^{f}, each of which is optionally substituted with 1-3 R¹²; wherein two R⁷ may be taken together with the atoms to which they are attached to form an optionally substituted cyclyl, heterocyclyl, aryl or heteroaryl ring;
R⁹ is C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d},—SR^{d′}—C(Y)R^{e} or —S(O)_{q}R^{f}, each of which is optionally substituted with 1-3 R¹²;
t is 1 to 4, wherein two R⁹ may be taken together with the atoms to which they are attached to form an optionally substituted cyclyl, heterocyclyl, aryl or heteroaryl ring;
each R¹¹ and R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d},—SR^{d′}, —C(Y)R^{e} or —S(O)_{q}R^{f}, each of which is optionally substituted with 1-3 R¹³;
R¹³ is independently C₁-C₈ alkyl, haloalkyl, halo, heterocyclyl, cyclyl, oxo or —C(Y)NR^{b}R^{b′};
Y is independently O or S;
q is 1 or 2; and
each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.

9. The compound of claim 8, wherein if X₂ is N and X₁, X₃, X₄ are CH, 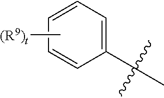
is not

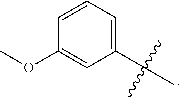 [SMILES: *c1cccc(OC)c1 ; "."]

10. The compound of claim 8, provided the compounds in Table X are excluded.

11. The compound of claim 8, wherein X₂ is N, and X₁, X₃, and X₄ are CH.

12. The compound of claim 8, wherein X₁ and X₃ are N, and X₂ and X₄ are CH.

13. The compound of claim 8, wherein R^{d} is methyl.

14. The compound of claim 8, wherein R⁹ is fluoro.

15. A compound of formula (III): 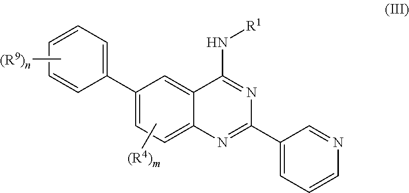
wherein:
R¹ is hydrogen, C₁-C₈ alkyl, halo C₁-C₈ alkyl, C₁-C₈ alkoxy C₁-C₈ alkyl, hydroxy C₁-C₈ alkyl, amino C₁-C₈ alkyl, oxazolyl, thiazolyl, isoxazolyl, pyridyl, pyrrolopyridyl, oxadiazolyl C₁-C₈ alkyl, pyridyl C₁-C₈ alkyl, oxazolyl C₁-C₈ alkyl, phenyl C₁-C₈ alkyl, —C(O)R^{e}, pyrrolidinyl, azetidinyl, indolinyl, piperidinyl, morpholinyl, piperazinyl, phenyl, C₃-C₈ cycloalkyl, C₃-C₈ cycloalkyl C₁-C₈ alkyl, benzoxazolyl, each of which is optionally substituted with 1-2 R⁷;
each R⁴ is independently hydrogen, C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹⁰;
m is 1 or 2;
each R⁷, R⁹, or R¹⁰ is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹² wherein two R⁹ may, together with the ring atoms to which they are attached, form a five or six-membered aryl, heteroaryl, cyclic, or heterocyclic;
n is 1, 2, or 3;
each R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹³;
each R¹³ is independently C₁-C₈ alkyl, haloalkyl, halo, heterocyclyl, cyclyl, oxo or —C(Y)NR^{b}R^{b′};
Y is independently O or S;
q is 1 or 2; and
each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.

16. The compound of claim 15, wherein
if R¹ is methyl or phenyl and R⁴ is methyl, then R⁹ is not fluoro, cyano, or methoxy; if formula (III) is formula (III′): 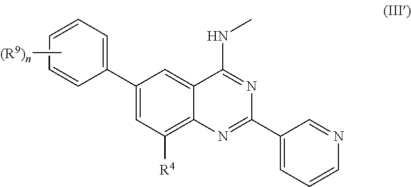
and R⁴ is fluoro or methoxy, then R⁹ is not fluoro or methoxy;
if formula (III) is formula (III″): 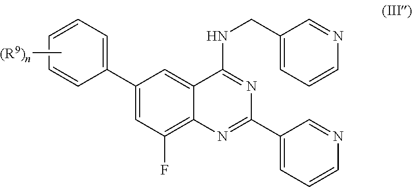
then R⁹ is not fluoro; and
the compound of formula (III) below 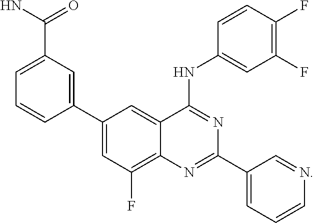 [SMILES: NC(=O)c1cccc(-c2cc(F)c3nc(-c4cccnc4)nc(Nc4ccc(F)c(F)c4)c3c2)c1]
is excluded.

17. The compound of claim 15, provided the compounds in Table X are excluded.

18. The compound of claim 15, wherein R¹ is C₁-C₈ alkyl.

19. The compound of claim 15, wherein R⁹ is halo.

20. A compound of formula (IV): 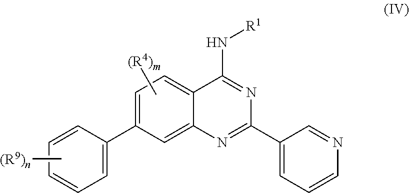
wherein:
R¹ is hydrogen, C₁-C₈ alkyl, halo C₁-C₈ alkyl, C₁-C₈ alkoxy C₁-C₈ alkyl, hydroxy C₁-C₈ alkyl, amino C₁-C₈ alkyl, oxazolyl, thiazolyl, isoxazolyl, pyridyl, pyrrolopyridyl, oxadiazolyl C₁-C₈ alkyl, pyridyl C₁-C₈ alkyl, oxazolyl C₁-C₈ alkyl, phenyl C₁-C₈ alkyl, —C(O)R^{e}, pyrrolidinyl, azetidinyl, indolinyl, piperidinyl, morpholinyl, piperazinyl, phenyl, C₃-C₈ cycloalkyl, C₃-C₈ cycloalkyl C₁-C₈ alkyl, benzoxazolyl, each of which is optionally substituted with 1-2 R⁷;
each R⁴ is independently hydrogen, C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹⁰;
m is 1 or 2;
each R⁷, R⁹, or R¹⁰ is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹², wherein two R⁹ may, together with the ring atoms to which they are attached, form a five or six-membered aryl, heteroaryl, cyclic, or heterocyclic;
n is 1, 2, or 3;
each R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹³;
each R¹³ is independently C₁-C₈ alkyl, haloalkyl, halo, heterocyclyl, cyclyl, oxo or —C(Y)NR^{b}R^{b′};
Y is independently O or S;
q is 1 or 2; and
each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.

21. The compound of claim 20, wherein if R¹ is methyl and R⁴ is methyl, then R⁹ is not fluoro, cyano, or methoxy.

22. The compound of claim 20, provided the compounds in Table X are excluded.

23. The compound of claim 20, wherein R¹ is C₁-C₈ alkyl.

24. The compound of claim 20, wherein R⁴ is fluoro.

25. A compound of formula (V): 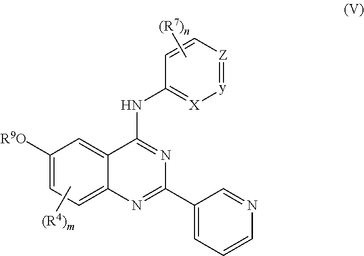
wherein:
one of X, Y, or Z is —N—, the rest being —CH— or —CR⁷—;
each R⁴ is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹⁰;
m is 0, 1, or 2;
each R⁷ or R¹⁰ is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹², wherein two R⁷ may, together with the ring to which they are attached, form a five or six-membered aryl or heteroaryl;
n is 0, 1, 2, or 3;
R⁹ is —CH₃ or —CH₂CH₃;
each R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹³;
each R¹³ is independently C₁-C₈ alkyl, haloalkyl, halo, heterocyclyl, cyclyl, oxo or —C(Y)NR^{b}R^{b′};
Y is independently O or S;
q is 1 or 2; and
each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.

26. The compound of claim 25, wherein the compound is not

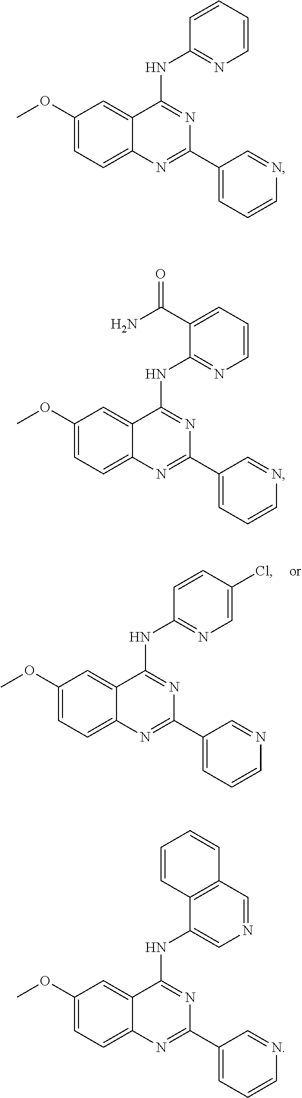 [SMILES: COc1ccc2nc(-c3cccnc3)nc(Nc3ccccn3)c2c1 ; COc1ccc2nc(-c3cccnc3)nc(Nc3ncccc3C(N)=O)c2c1 ; COc1ccc2nc(-c3cccnc3)nc(Nc3ccc(Cl)cn3)c2c1 ; COc1ccc2nc(-c3cccnc3)nc(Nc3cncc4ccccc34)c2c1]

27. The compound of claim 25, provided the compounds in Table X are excluded.

28. The compound of claim 25, wherein R⁷ is halo.

29. The compound of claim 25, wherein m is 0.

30. A compound of formula (VI): 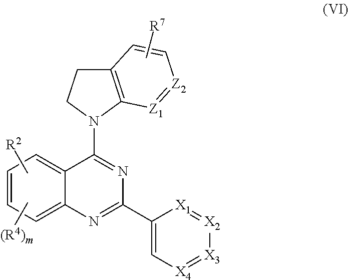
or a salt thereof,
wherein:
one or two of X¹, X², X³, and X⁴ are N and the others are CH;
Z₁ and Z₂ are independently N or CH;
m is 1, 2 or 3;
R² is halo, —OR^{d}, aryl or heteroaryl, wherein the aryl or heteroaryl is optionally substituted with 1-5 R⁹;
each R⁴ is independently hydrogen, C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d},—SR^{d′}, —C(Y)R^{e} or —S(O)_{q}R^{f}, each of which is optionally substituted with 1-3 R¹⁰;
each R⁷, R⁹, and R¹⁰ is independently C₁-C₈ alkyl, C₂-C₈ alkenyl, C₂-C₈ alkynyl, aryl, heteroaryl, cyclyl, heterocyclyl, arylalkyl, heteroarylalkyl, cyclylalkyl, heterocyclylalkyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹²;
each R¹² is independently C₁-C₈ alkyl, C₂-C₈ alkenyl, C₂-C₈ alkynyl, aryl, heteroaryl, cyclyl, heterocyclyl, arylalkyl, heteroarylalkyl, cyclylalkyl, heterocyclylalkyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d},—SR^{d′}, —C(Y)R^{e} or —S(O)_{q}R^{f},each of which is optionally substituted with 1-3 R¹³;
R¹³ is independently C₁-C₈ alkyl, haloalkyl, halo, heterocyclyl, cyclyl, oxo or —C(Y)NR^{b}R^{b′};
Y is independently O or S;
q is 1 or 2; and
each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.

31. The compound of claim 30, wherein if Z₁ and Z₂ are both CH, R² is not —Cl or —OR^{d}.

32. The compound of claim 30, provided the compounds in Table X are excluded.

33. The compound of claim 30, wherein Z₁ is N.

34. The compound of claim 30, wherein R² is aryl.

35. The compound of claim 30, wherein R² is —Br or —I.

36. The compound of claim 30, wherein X₂ is N, and X₁, X₃, and X₄ are CH.

37. A compound of formula (VII): 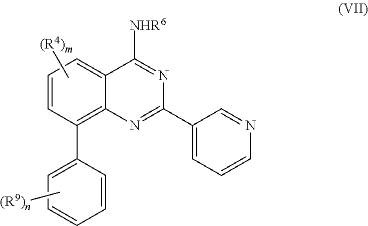
or a salt thereof,
wherein:
m is 1, 2 or 3;
n is 1, 2, 3 or 4;
each R⁴ is independently hydrogen, C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹⁰;
R⁶ is hydrogen, C₁-C₈ alkyl, C₂-C₈ alkenyl, or C₂-C₈ alkynyl, each of which is optionally substituted with 1-3 R¹¹;
each R⁹ and R¹⁰ is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d},—SR^{d′}, —C(Y)R^{e} or —S(O)_{q}R^{f}, each of which is optionally substituted with 1-3 R¹²;
each R¹¹ and R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹³;
R¹³ is independently C₁-C₈ alkyl, haloalkyl, halo, heterocyclyl, cyclyl, oxo or —C(Y)NR^{b}R^{b′};
Y is independently O or S;
q is 1 or 2; and
each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.

38. The compound of claim 37, wherein if R⁴ is hydrogen, 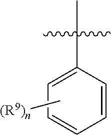
is not

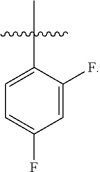 [SMILES: *c1ccc(F)cc1F]

39. The compound of claim 37, provided the compound is not in Table X.

40. The compound of claim 37, wherein R⁴ is —OCH₃.

41. The compound of claim 37, wherein R⁹ is —F.

42. A compound of formula (VIII): 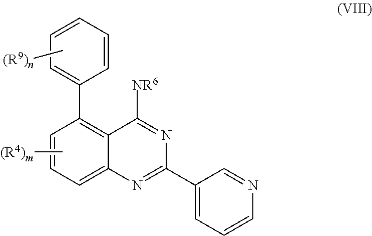
or a salt thereof,
wherein:
m is 1, 2 or 3;
n is 1, 2, 3 or 4;
each R⁴ is independently hydrogen, C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹⁰;
R⁶ is hydrogen, C₁-C₈ alkyl, C₂-C₈ alkenyl, or C₂-C₈ alkynyl, each of which is optionally substituted with 1-3 R¹¹;
each R⁹ and R¹⁰ is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹²;
each R¹¹ and R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —SR^{d′}, —C(Y)R^{e} or —S(O)_{q}R^{f}, each of which is optionally substituted with 1-3 R¹³;
R¹³ is independently C₁-C₈ alkyl, haloalkyl, halo, heterocyclyl, cyclyl, oxo or —C(Y)NR^{b}R^{b′};
Y is independently O or S;
q is 1 or 2; and
each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.

43. The compound of claim 42, provided the compound is not in Table X.

44. The compound of claim 42, wherein R⁹ is —F.

45. A compound of formula (IX) or (IX′): 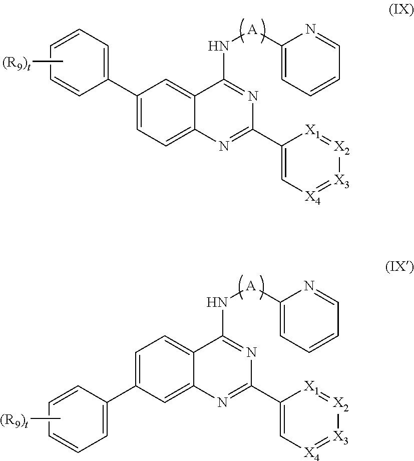
or a salt thereof,
wherein:
A is C₁-C₄ alkylene, optionally substituted with R¹¹;
one or two of X¹, X², X³, and X⁴ are N and the others are CH,
R⁹ is C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹²;
t is 1 to 4, wherein two R⁹ may be taken together with the ring atoms to which they are attached to form an optionally substituted cyclyl, heterocyclyl, aryl or heteroaryl ring;
each R¹¹ and R¹² is independently C₁-C₈ alkyl, C₂-C₈ alkenyl, C₂-C₈ alkynyl, halo, haloalkyl, haloalkoxy, aminoalkyl, alkylaminoalkyl, dialkylaminoalkyl, hydroxyalkyl, alkoxyalkyl, oxo, thioxo, —CN, —NO₂, —C(O)OR^{a}, —C(Y)NR^{b}R^{b′}, —NR^{c}C(Y)R^{c′}, —NR^{b}R^{b′}, —OC(O)NR^{b}R^{b′}, —NR^{c}C(O)OR^{c′}, —SO₂NR^{b}R^{b′}, —NR^{c}SO₂R^{c′}, —NR^{c}C(Y)NR^{b}R^{b′}, —OR^{d}, —SR^{d′}, —C(Y)R^{e} or —S(O)_{q}R^{f}, each of which is optionally substituted with 1-3 R¹³;
R¹³ is independently C₁-C₈ alkyl, haloalkyl, halo, heterocyclyl, cyclyl, oxo or —C(Y)NR^{b}R^{b′};
alternatively, R¹³ on R¹¹ may connect to the carbon atom of A to which R¹¹ bonds to form a C₃₋₆ cycloalkyl. Y is independently O or S;
q is 1 or 2; and
each R^{a}, R^{b}, R^{b′}, R^{c}, R^{c′}, R^{d}, R^{d′}, R^{e} and R^{f} is independently hydrogen, C₁-C₈ alkyl, C₂-C₈ alkenyl, C₂-C₈ alkynyl, acyl, haloalkyl, alkoxyalkyl, alkylaminoalkyl, dialkylaminoalkyl, cyclyl, heterocyclyl, aryl, heteroaryl, cyclylalkyl, heterocyclylalkyl, arylalkyl or heteroarylalkyl.

46. The compound of claim 45, wherein if X₂ is N and X₁, X₃, X₄ are CH, R⁹ is not —F or —OR^{d}.

47. The compound of claim 45, provided the compound is not in Table X.

48. The compound of claim 45, wherein A is —CH₂—.

49. The compound of claim 45, wherein A is —C(CH₃)H—.

50. The compound of claim 45, wherein R⁹ is —F.

51. A compound disclosed herein.

52. The compound according to claim 8, wherein
R¹ is C₁-C₈ alkyl, halo C₁-C₈ alkyl, C₁-C₈ alkoxy C₁-C₈ alkyl, hydroxyl C₁-C₈ alkyl, amino C₁-C₈ alkyl, oxadiazolyl C₁-C₈ alkyl, oxazolyl C₁-C₈ alkyl, —C(O)R^{e}, C₃-C₈ cycloalkyl, pyrrolidinyl, azetidinyl, piperidinyl, morpholinyl or piperazinyl, each of which is optionally substituted with 1-2 R⁷;
R⁶ is hydrogen or C₁-C₈ alkyl;
R⁷ is C₁-C₈ alkyl, C₁-C₈ alkoxy, halo, halo C₁-C₈ alkyl, C₁-C₈ alkylamino, di C₁-C₈ alkylamino, oxo, —C(O)NR^{b}R^{b′} or —C(O)R^{e}, each of which is optionally substituted with R¹²;
R⁹ is C₁-C₈ alkyl, C₁-C₈ alkoxy, oxazolyl, thiazolyl C₃-C₈ cycloalkyl, halo, cyano or —C(O)NR^{b}R^{b′}, each of which is optionally substituted with 1-2 R¹²;
R¹² is C₁-C₈ alkoxy or —C(O)NR^{b}R^{b′} and
each R^{a}, R^{b}, R^{b′}, R^{c}, R^{c′}, R^{d}, R^{d′}, R^{e} and R^{f} is independently hydrogen or C₁-C₈ alkyl.

53. The compound according to claim 25, wherein
m is 0;
R⁷ is C₁-C₈ alkyl, halo, haloalkyl, —CN, —C(O)NR^{b}R^{b′} or —OR^{d}, each of which is optionally substituted with 1-3 R¹², wherein two R⁷ may, together with the ring to which they are attached, form benzoxazolyl;
n is 0, 1 or 2
R⁹ is —CH₃ or —CH₂CH₃;
R¹² is C₁-C₈ alkyl or halo;
each R^{a}, R^{b}, R^{b′}, R^{c}, R^{c′}, R^{d}, R^{d′}, R^{e} and R^{f} is independently hydrogen or C₁-C₈ alkyl.

54. The compound according to claim 30, wherein
m is 1, 2 or 3;
R² is halo, —OR^{d}, piperazinyl, phenyl, pyridyl, pyrimidinyl or benzodioxolyl, wherein the phenyl is optionally substituted with 1-2 R⁹;
R⁴ is hydrogen or C₁-C₈ alkyl;
R⁷ is C₁-C₈ alkyl, halo, —NO₂, —NR^{c}C(O)R^{c′} or —OR^{d};
R⁹ is C₁-C₈ alkyl, halo, —CN, —NO₂, —C(O)NR^{b}R^{b′}, —NR^{c}C(O)R^{c′} or —NR^{b}R^{b′}; and
each R^{a}, R^{b}, R^{b′}, R^{c}, R^{c′}, R^{d}, R^{d′}, R^{e} and R^{f} is independently hydrogen or C₁-C₈ alkyl.

55. The compound according to claim 45, wherein
R⁹ is C₁-C₈ alkyl, halo, —CN or —OR^{d};
t is 1 to 4, wherein two R⁹ may be taken together with the ring atoms to which they are attached to form an optionally substituted indolyl, indazolyl or benzothienyl;
R¹¹ is C₁-C₈ alkyl; and
R^{d} is C₁-C₈ alkyl.

56. The compound according to claim 15, wherein
R¹ is C₁-C₈ alkyl;
R⁴ is hydrogen, halo, haloalkyl, haloalkoxy or —OR^{d};
m is 1;
R⁹ is halo, —CN, —C(O)NR^{b}R^{b′} or —OR^{d};
n is 1 or 2; and
each R^{b}, R^{b′} and R^{d} is independently C₁-C₈ alkyl.

57. The compound according to claim 20, wherein
R¹ is C₁-C₈ alkyl;
R⁴ is C₁-C₈ alkyl or halo;
m is 1;
R⁹ is C₁-C₈ alkyl, halo, haloalkyl, —CN or —OR^{d}, each of which is optionally substituted with 1 R¹², wherein two R⁹ may, together with the ring atoms to which they are attached, form indazolyl or benzothienyl;
R¹² is C₁-C₈ alkyl; and
R^{d} is C₁-C₈ alkyl.

58. The compound according to claim 37, wherein
m is 1;
n is 1 or 2;
R⁴ is hydrogen, or —OR^{d};
R⁹ is halo, —CN or —OR^{d}; or
each R^{d} is C₁-C₈ alkyl.

59. The compound according to claim 1, which is

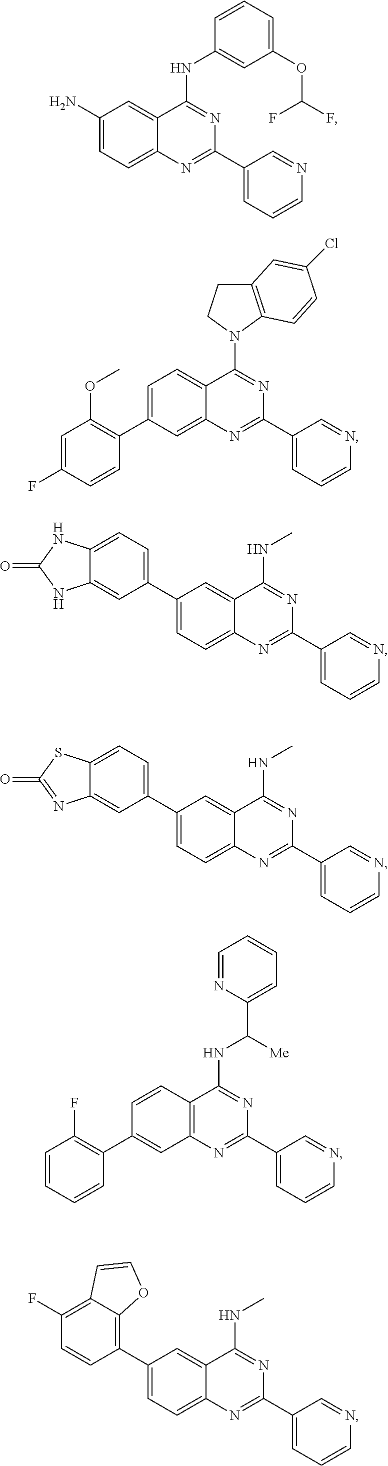

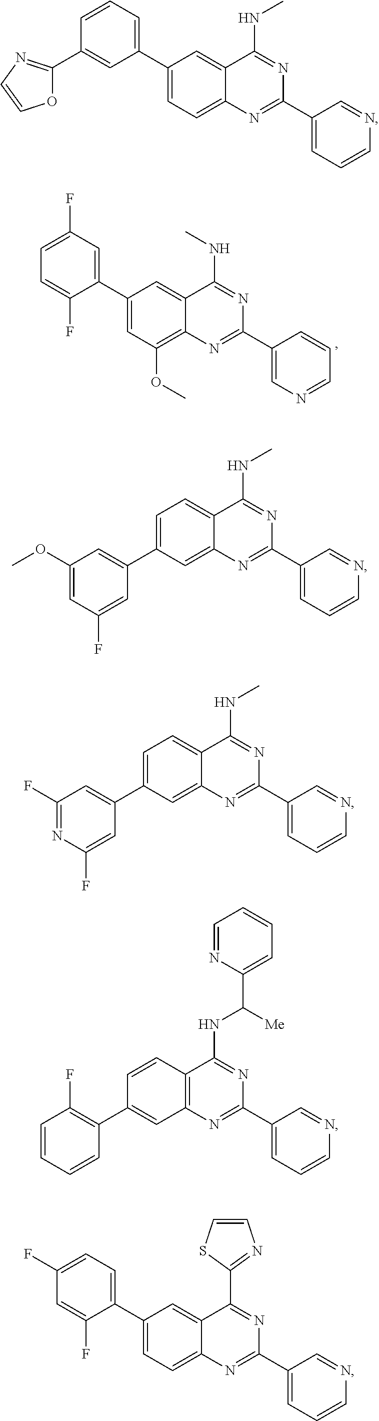 [SMILES: CNc1nc(-c2cccnc2)nc2ccc(-c3cccc(-c4ncco4)c3)cc12 ; CNc1nc(-c2cccnc2)nc2c(OC)cc(-c3cc(F)ccc3F)cc12 ; CNc1nc(-c2cccnc2)nc2cc(-c3cc(F)cc(OC)c3)ccc12 ; CNc1nc(-c2cccnc2)nc2cc(-c3cc(F)nc(F)c3)ccc12 ; CC(Nc1nc(-c2cccnc2)nc2cc(-c3ccccc3F)ccc12)c1ccccn1 ; Fc1ccc(-c2ccc3nc(-c4cccnc4)nc(-c4nccs4)c3c2)c(F)c1]

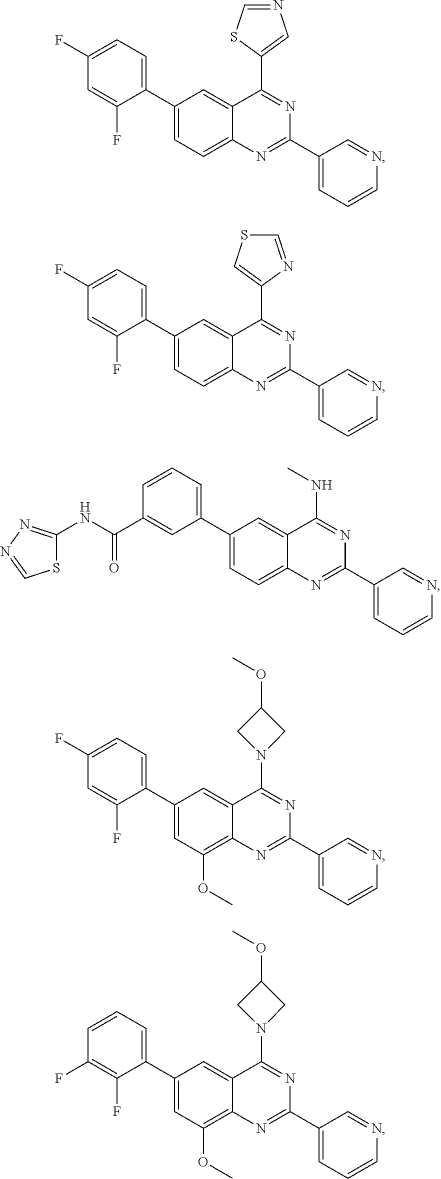

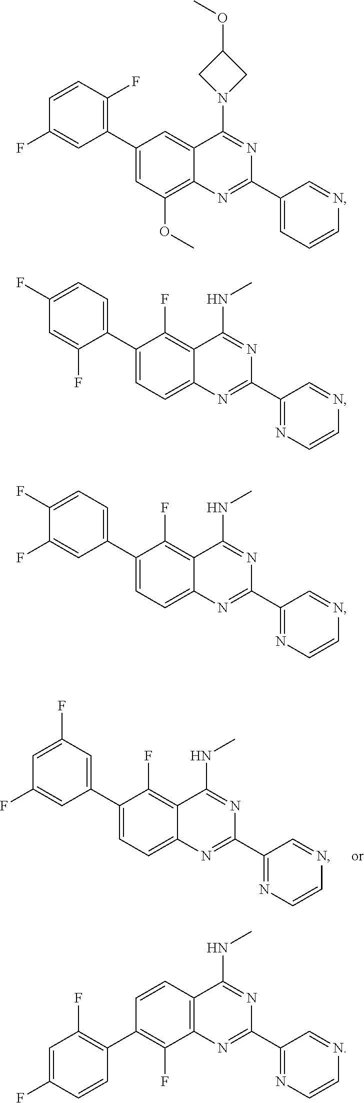

60. A pharmaceutical composition comprising the compound or a salt thereof according to claim 1 as an active ingredient and a pharmaceutically acceptable carrier.

61. The pharmaceutical composition according to claim 60 for preventing or treating central nervous system diseases.

62. The pharmaceutical composition according to claim 61 for treating or preventing central nervous system disorders selected from the group consisting of schizophrenia; refractory, intractable or chronic schizophrenia; emotional disturbance; psychotic disorder; mood disorder; bipolar I type disorder; bipolar II type disorder; depression; endogenous depression; major depression; melancholy and refractory depression; dysthymic disorder; cyclothymic disorder; panic attack; panic disorder; agoraphobia; social phobia; obsessive-compulsive disorder; post-traumatic stress disorder; generalized anxiety disorder; acute stress disorder; hysteria; somatization disorder; conversion disorder; pain disorder; hypochondriasis; factitious disorder; dissociative disorder; sexual dysfunction; sexual desire disorder; sexual arousal disorder; erectile dysfunction; anorexia nervosa; bulimia nervosa; sleep disorder; adjustment disorder; alcohol abuse; alcohol intoxication; drug addiction; stimulant intoxication; narcotism; anhedonia; iatrogenic anhedonia; anhedonia of a psychic or mental cause; anhedonia associated with depression; anhedonia associated with schizophrenia; delirium; cognitive impairment; cognitive impairment associated with Alzheimer's disease, Parkinson's disease and other neurodegenerative diseases; cognitive impairment caused by Alzheimer's disease; Parkinson's disease and associated neurodegenerative diseases; cognitive impairment of schizophrenia; cognitive impairment caused by refractory, intractable or chronic schizophrenia; vomiting; motion sickness; obesity; migraine; pain (ache); mental retardation; autism disorder (autism); Tourette's disorder; tic disorder; attention-deficit/hyperactivity disorder; conduct disorder; and Down's syndrome.

63. A process for producing a pharmaceutical composition comprising mixing a compound or a salt thereof according to claim 1 with a pharmaceutically acceptable carrier.

64. Use of a compound or a salt thereof according to claim 1 as a drug.

65. Use of the compound or a salt thereof according to claim 1 as a STEP inhibitor.

66. A method of treating a disorder that would benefit by the modulation of STEP in a subject, the method comprising administering to a compound or a salt thereof according to claim 1.

67. The method of claim 66, wherein the disorder is schizophrenia.

68. The method of claim 66, wherein the disorder is cognitive deficit.

69. The method of claim 66, wherein the compound or a salt thereof is administered in combination with an additional therapeutic agent.

70. The method of claim 66, wherein the additional therapeutic agent is an atypical antipsychotic.

71. The method of claim 66, wherein the additional therapeutic agent is selected from the group consisting of aripiprazole, clozapine, ziprasidone, risperidone, quetiapine, olanzapine, amisulpride, asenapine, iloperidone, melperone, paliperidone, perospirone, sertindole and sulpiride.

72. The method of claim 66, wherein the additional therapeutic agent is a typical antipsychotic.

73. The method of claim 66, wherein the additional therapeutic agent is selected from the group consisting of haloperidol, molindone, loxapine, thioridazine, molindone, thiothixene, pimozide, fluphenazine, trifluoperazine, mesoridazine, chlorprothixene, chlorpromazine, perphenazine, triflupromazine and zuclopenthixol.

74. A kit comprising a composition comprising a compound or a salt thereof according to claim 1 and an acceptable carrier.

75. A kit comprising a pharmaceutical composition comprising a compound or a salt thereof according to claim 1 and a pharmaceutically acceptable carrier.
